(19) United States
(12) Patent Application Publication
(10) Pub. No.: US 2026/0258414 A1
(43) Pub. Date: Sep. 3, 2026

(54) COMPOUNDS TARGETING PMP22 FOR THE TREATMENT OF CHARCOT-MARIE-TOOTH DISEASE

(71) Applicant: Novartis AG, Basel (CH)

(72) Inventors: Charles ALLERSON, San Diego, CA (US); Arthur T. SUCKOW, San Diego, CA (US)

(21) Appl. No.: 19/331,332

(22) Filed: Sep. 17, 2025

Related U.S. Application Data
(63) Continuation of application No. 18/660,600, filed on May 10, 2024, now Pat. No. 12,497,616.
Application No. 18/660,600 is a continuation of application No. PCT/US2022/080012, filed on Nov. 17, 2022.
(60) Provisional application No. 63/280,773, filed on Nov. 18, 2021.

(51) Int. Cl. C12N 15/113; A61P 25/28

(52) U.S. Cl. CPC C12N 15/113; A61P 25/28; C12N 2310/11; C12N 2320/30

(57) ABSTRACT

Provided herein are compounds for inhibiting peripheral myelin protein 22 (PMP22) mRNA. Also provided herein are methods of using such compounds for the treatment of Charcot-Marie-Tooth disease.

### CROSS-REFERENCE TO RELATED APPLICATIONS

This application is a continuation of U.S. patent application Ser. No. 18/660,600 filed May 10, 2024, which is a continuation of International Patent Application No. PCT/US2022/080012 filed Nov. 17, 2022, which claims the benefit of U.S. Provisional Application No. 63/280,773 filed Nov. 18, 2021, the contents of each of which are hereby incorporated herein in their entirety and for all purposes.

### STATEMENT REGARDING FEDERALLY SPONSORED RESEARCH

This invention was made with government support under grant number 1R43NS119090-O1A1 awarded by the National Institutes of Health. The government has certain rights in the invention.

### REFERENCE TO AN ELECTRONIC SEQUENCE LISTING

The contents of the electronic sequence listing (PAT059574-US-CNT01_Sequence_Listing_ST26.xml; Size: 1,512,914 bytes; and Date of Creation: Sep. 17, 2025) are hereby incorporated by reference in their entirety.

### FIELD

The present disclosure relates to compounds and methods for the treatment of Charcot-Marie-Tooth disease. More specifically, the present disclosure relates to inhibitors of PMP22 and their use in the treatment of Charcot-Marie-Tooth disease.

### BACKGROUND

Charcot-Marie-Tooth (CMT) disease is an inherited peripheral neuropathy characterized by slowly progressive muscle atrophy. CMT is one of the most common inherited neurological disorders, affecting approximately 150,000 people across the United States and Europe. There are several subtypes of CMT disease, each having a distinct genetic cause. The most common form of CMT, accounting for as many as 60% of cases, is CMT type TA (CMT1A), which results from an excess of peripheral myelin protein 22 (PMP22) protein due to the duplication of one PMP22 alelle.

The PMP22 protein is a major component of myelin that comprises between two and five percent of the myelin that insulates peripheral nerves. While the exact role of PMP22 is not known, there is evidence that overexpression of PMP22 alters the growth and differentiation of Schwann cells, the cells responsible for producing the myelin sheath around neurons. The myelin sheath is a protective layer of lipids and proteins that serves as insulation around nerve axons and facilitates the ability to rapidly conduct nerve signals. In addition to causing deficiencies in the ability to generate new myelin, the presence of excess PMP22 protein in the myelin sheath has been reported to directly destabilize the myelin sheath, leading to increased rates of demyelination. Defects in the myelin sheath reduce the speed that nerve signals can be propagated along nerves, known as the motor nerve conduction velocity, or MNCV. This in turn leads to progressive muscle atrophy in the peripheral limbs resulting in muscle weakness, structural abnormalities in the feet, and abnormal spinal curvature.

Overexpression of PMP22 in mice results in symptoms characteristic of CMT1A disease, including muscle weakness, gait abnormalities, myelination defects, and reduced nerve conduction velocities. Under the control of a conditionally regulated promoter, PMP22 overexpression caused demyelination of neurons, which was reversed upon subsequent suppression of PMP22 expression. Within one week, new myelin sheath formation was evident and within 12 weeks, myelinated neurons were similar to those present in transgenic mice in which PMP22 expression was not suppressed.

Mice harboring three to four copies of the human PMP22 gene develop pathologies similar to those observed in subjects with CMT1A and as such, these mice are used as an experimental model of CMT1A. In this model, treatment with an antisense oligonucleotide complementary to human PMP22 lowered PMP22 mRNA levels and led to restoration of myelination, improvement of MNCV and reversal of other neuropathy endpoints. However, the high doses required in the mouse model translate to dosages that are unlikely to be tolerated in human subjects, thus antisense oligonucleotides targeted to PMP22 have not advanced to development as a treatment for CMT1A.

While a small number of potential therapies are being evaluated in clinical trials, an effective treatment for any CMT disease, including CMT1A, has yet to be identified. Current care consists of physical therapy, occupational therapy and orthopedic devices to help patients cope with disabling symptoms, and pain-relieving drugs for patients with severe pain. Accordingly, there remains an unmet medical need for therapeutic agents for the treatment of CMT1A.

### SUMMARY

Provided herein are, inter alia, nucleic acid compounds targeted to the peripheral myelin protein 22 (PMP22) mRNA.

In embodiments, provided is a compound comprising an antisense strand and a sense strand hybridized to form a double-stranded nucleic acid, wherein each of the antisense strand and sense strands is 15 to 25 nucleotides in length, the nucleotide sequence of the antisense strand is at least 90% complementary to the nucleotide sequence of the PMP22 mRNA (SEQ ID NO: 1170), and the nucleotide sequence of the sense strand has no more than two mismatches to the nucleotide sequence of the antisense strand.

In embodiments, each of the antisense strand and sense strands is 15 to 25 nucleotides in length, the nucleotide sequence of the antisense strand comprises at least 15 contiguous nucleotides of any one of SEQ ID NOs 491, 492, 493, 494, 495, 497, 498, 503, 504, 506, 510, 511, 514, 515, 516, 518, 524, 526, 529, 531, 532, 533, 534, 535, 536, 538, 539, 540, 541, 542, 543, 545, 546, 547, 548, 550, 553, 554, 556, 558, 559, 560, 561, 563, 567, 569, 575, 576, 579, 580, 581, 582, 583, 585, 590, 591, 595, 597, 600, 605, 609, 610, 618, 622, 623, 628, 630, 631, 633, 635, 637, 639, 641, 642, 643, 644, 645, 1112, 1113, 1114, 1115, 1116, 1117, 1118, 1119, 1120, 1122, 1124, 1125, 1126, 1127, 1128, 1129, 1130, 1131, 1132, 1133, 1134, 1135, 1136, 1137, 1138, 1139, 1140, 1141, 1142, 1143, 1144, 1145, 1146, 1147, 1148, 1149, 1150, 1151, 1152, 1153, 1154, 1155, 1156, 1157, 1158, 1159, 1160, 1161, 1162, 1163, 1164, 1165, 1166, 1167, 1168, 1169, 1118, 1121, 1123, 1126, and 1144, and the nucleotide sequence of the sense strand has no more than two mismatches to the nucleotide sequence of the antisense strand.

In embodiments, the antisense strand and the sense strand are not covalently linked.

In embodiments, at least one nucleotide of the antisense strand is a modified nucleotide. In embodiments, at least one nucleotide of the sense strand is a modified nucleotide. In embodiments, the 5′-terminal nucleotide of the antisense strand comprises a 5′-VP modification.

In embodiments, the antisense strand is 21 to 23 nucleotides in length. In embodiments, the sense strand is 21 to 23 nucleotides in length.

In embodiments, the hybridization of the antisense strand to the sense strand forms at least one blunt end. In embodiments, at least one strand comprises a 3′ nucleotide overhang of one to five nucleotides.

In embodiments, the compound comprises a ligand covalently linked to the antisense strand or the sense strand.

In embodiments, the compound has the structure:

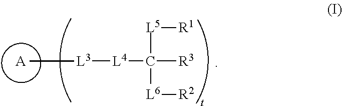

A is the sense strand or the antisense strand. t is an integer from 1 to 5.

L³ and L⁴ are independently a bond, —N(R²³)—, —O—, —S—, —C(O)—, —N(R²³)C(O)—, —C(O)N(R²⁴)—, —N(R²³)C(O)N(R²⁴)—, —C(O)O—, —OC(O)—, —N(R²³)C(O)O—, —OC(O)N(R²⁴)—, —OPO₂—O—, —O—P(O)(S)—O—, —O—P(O)(R²⁵)—O—, —O—P(S)(R²⁵)—O—, —O—P(O)(NR²³R²⁴)—N—, —O—P(S)(NR²³R²⁴)—N—, —O—P(O)(NR²³R²⁴)—O—, —O—P(S)(NR²³R²⁴)—O—, —P(O)(NR²³R²⁴)—N—, —P(S)(NR²³R²⁴)—N—, —P(O)(NR²³R²⁴)—O—, —P(S)(NR²³R²⁴)—O—, —S—S—, substituted or unsubstituted alkylene, substituted or unsubstituted heteroalkylene, substituted or unsubstituted cycloalkylene, substituted or unsubstituted heterocycloalkylene, substituted or unsubstituted arylene or substituted or unsubstituted heteroarylene. Each R²³, R²⁴ and R²⁵ is independently hydrogen or unsubstituted C₁-C₁₀ alkyl.

L⁵ is -L^{5A}-L^{5B}-L^{5C}-L^{5D}-L^{5E}-L⁶ is -L^{6A}-L^{6B}-L^{6C}-L^{6D}-L^{6E}-L^{5A}, L^{5B}, L^{5C}, L^{5D}, L^{5E}, L^{6A}, L^{6B}, L^{6C}, L^{6D}, and L^{6E} are independently a bond, —NH—, —O—, —S—, —C(O)—, —NHC(O)—, —NHC(O)NH—, —C(O)O—, —OC(O)—, —C(O)NH—, substituted or unsubstituted alkylene, substituted or unsubstituted heteroalkylene, substituted or unsubstituted cycloalkylene, substituted or unsubstituted heterocycloalkylene, substituted or unsubstituted arylene or substituted or unsubstituted heteroarylene; and each R²³, R²⁴ and R²⁵ is independently hydrogen or unsubstituted C₁-C₁₀ alkyl.

R¹ and R² are independently unsubstituted C₁-C₂₅ alkyl, wherein at least one of R¹ and R² is unsubstituted C₉-C₁₉ alkyl. R³ is hydrogen, —NH₂, —OH, —SH, —C(O)H, —C(O)NH₂, —NHC(O)H, —NHC(O)OH, —NHC(O)NH₂, —C(O)OH, —OC(O)H, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In embodiments, provided herein is a pharmaceutical composition comprising the compound as described herein.

In embodiments, provided herein are methods for inhibiting the expression of peripheral myelin protein 22 (PMP22) mRNA in a cell, comprising contacting the cell with a compound of provided herein, thereby inhibiting the expression of PMP22 mRNA in the cell

In embodiments, provided herein are methods for inhibiting the expression of peripheral myelin protein 22 (PMP22) mRNA in a subject, comprising administering to the subject an effective amount of a compound or pharmaceutical composition provided herein, thereby inhibiting the expression of peripheral myelin protein 22 (PMP22) mRNA.

In embodiments, provided herein are methods for increasing myelination and/or slowing the loss of myelination in a subject, comprising administering to the subject an effective amount of a compound or pharmaceutical composition provided herein.

In embodiments, provided herein are methods for treating Charcot-Marie-Tooth disease (CMT) in a subject, comprising administering to the subject an effective amount of a compound or pharmaceutical composition provided herein. In embodiments, the Charcot-Marie-Tooth disease (CMT) is Charcot-Marie-Tooth disease Type 1A (CMT1A).

### BRIEF DESCRIPTION OF THE DRAWINGS

FIG. 1 shows the mean percent hPMP22 mRNA remaining in the sciatic and brachial plexus nerves of C3-PMP22 mice, following treatment with 10 mg/kg DT-000812 or 30 mg/kg for a period of 12 weeks.

FIG. 2 shows the mean motor nerve conduction velocity (MNCV) in wild-type mice treated with PBS, and C3-PMP22 mice treated with PBS, 10 mg/kg DT-000812, and 30 mg/kg DT-000812 at the indicated timepoints.

FIG. 3A shows the mean compound muscle action potentials in wild-type mice treated with PBS, and C3-PMP22 mice (CMT1A mice) treated with PBS, 10 mg/kg DT-000812, and 30 mg/kg DT-000812, at the indicated timepoints.

FIG. 3B shows representative CMAP traces recorded from wild-type mice treated with PBS, and C3-PMP22 mice (CMT1A mice) treated with PBS, 10 mg/kg DT-000812, and 30 mg/kg DT-000812, for a period of 12 weeks.

FIG. 4 shows the mean proportion of unmyelinated axons in wild-type mice treated with PBS and C3-PMP22 mice treated with PBS, 10 mg/kg DT-000812, and 30 mg/kg DT-000812, for a period of 12 weeks.

FIG. 5 shows representative images of nerve cross sections in mice treated with PBS, 10 mg/kg DT-000812, and 30 mg/kg DT-000812, for a period of 12 weeks.

FIG. 6 shows representative CMAP traces recorded from wild-type mice treated with PBS, C3-PMP22 mice (CMT1A mice) treated with PBS, 3 mg/kg DT-001252, 10 mg/kg DT-001252, and 30 mg/kg DT-001252, for a period of 12 weeks. Also shown is the mean CMAP for each treatment group after 12 weeks of treatment.

FIG. 7 shows the mean percentage of unmyelinated axons in wild-type mice treated with PBS and C3-PMP22 mice (CMT1A mice) treated with PBS, 30 mg/kg DT-000812, 3 mg/kg DT-001252, 10 mg/kg DT-001252, and 30 mg/kg DT-001252, for a period of 12 weeks.

### DETAILED DESCRIPTION

### Definitions

Unless defined otherwise, all technical terms, scientific terms, abbreviations, chemical structures, and chemical formulae used herein have the same meaning as is commonly understood by one of ordinary skill in the art. The chemical structures and formulae set forth herein are constructed according to the standard rules of chemical valency known in the chemical arts. All patents, applications, published applications, and other publications referenced herein are incorporated by reference in their entirety unless stated otherwise. Unless otherwise indicated, conventional methods of mass spectroscopy, NMR, HPLC, protein chemistry, biochemistry, recombinant DNA techniques, and pharmacology are employed. Furthermore, use of the term “including” as well as other forms, such as “include”, “includes,” and “included,” is not limiting. As used in this specification, whether in a transitional phrase or in the body of the claim, the terms “comprise(s)” and “comprising” are to be interpreted as having an open-ended meaning. That is, the terms are to be interpreted synonymously with the phrases “having at least” or “including at least.” When used in the context of a process, the term “comprising” means that the process includes at least the recited steps, but may include additional steps. When used in the context of a compound, composition, or device, the term “comprising” means that the compound, composition, or device includes at least the recited features or components, but may also include additional features or components.

Where substituent groups are specified by their conventional chemical formulae, written from left to right, they equally encompass the chemically identical substituents that would result from writing the structure from right to left, e.g., —CH₂O— is equivalent to —OCH₂—.

“Charcot-Marie-Tooth disease” or “CMT” means an inherited peripheral neuropathy affecting both motor and sensory nerves. CMT is characterized by muscle weakness and atrophy in the legs and arms, foot deformities and loss of sensation and/or numbness. CMT disease includes the CMT1A subtype, among others.

“Charcot-Marie-Tooth disease Type TA” or CMT1A means the subtype of CMT that results from a duplication of one PMP22 allele, resulting in three copies of the PMP22 gene in subjects.

“Nerve conduction velocity” means the speed with which an electrical impulse moves through a nerve. In embodiments, nerve conduction velocity is motor nerve conduction velocity. In embodiments, nerve conduction velocity is sensory nerve conduction velocity. In embodiments, nerve conduction velocity may be determined by an electroneuroagraphy, i.e. a nerve conduction study.

“Compound muscle action potential” is a is a quantitative measure of the amplitude of the electrical impulses that are transmitted to muscle, correlating with the number of muscle fibers that can be activated. In embodiments, compound muscle action potential is determined by electromyography (EMG).

“Improve” means to lessen the severity of a symptom and/or clinical indicator of a disease.

“Slow the progression of” means to reduce the rate at which a symptom and/or clinical indicator of a disease becomes more severe.

“Therapeutically effective amount” means an amount sufficient for a compound to provide a therapeutic benefit to a subject.

“Subject” used herein means a human or non-human animal selected for treatment or therapy. In embodiments, a subject is a human.

“Administration” means providing a pharmaceutical agent or composition to a subject, and includes administration performed by a medical professional and self-administration. In embodiments, administration is intravenous administration. In embodiments, administration is subcutaneous administration.

“Treating” or “treatment” means the administration of one or more pharmaceutical agents to a subject to achieve a desired clinical result, including but not limited to the alleviation, improvement, or slowing of the progression of at least one clinical indicator and/or symptom of a disease in a subject.

“Delay the onset of” means to delay the development of a condition or disease in a subject who is at risk for developing the disease or condition. In embodiments, a subject at risk for developing a disease or condition is identified using clinical assessments similar to those used to diagnose the disease or condition. For example, a subject at risk for developing CMT1A may be identified by genetic testing for amplication of the PMP22 gene. In embodiments, a subject at risk for developing the disease or condition receives treatment similar to the treatment received by a subject who already has the disease or condition.

“Effective amount” means an amount sufficient for a compound that, when administered to a subject, is sufficient to effect treatment of a disease in the subject. An effective amount may vary depending on one or more of the potency of the compound, its mode of administration, the severity of the disease in the subject, concomitant pharmaceutical agents the subject is receiving, and characteristics of the subject such as the subject's medical history, age, and weight.

“Pharmaceutical salt” means a salt form of a compound that retains the biological effectiveness and properties of a compound and does not have undesired effects when administered to a subject.

“Compound” means a molecule comprising linked monomeric nucleotides. A compound may have one or more modified nucleotides. In embodiments, a compound comprises a double-stranded nucleic acid. In embodiments, a compound comprises a single-stranded nucleic acid. A compound may be provided as a pharmaceutical salt. A compound may be provided as a pharmaceutical composition.

“Oligonucleotide” means a polymer of linked monomeric nucleotides. One or more nucleotides of an oligonucleotide may be a modified nucleotide.

“Double-stranded nucleic acid” means a first nucleotide sequence hybridized to a second nucleotide sequence to form a duplex structure. Double-stranded nucleic acids include structures formed from annealing a first oligonucleotide to a second, complementary oligonucleotide, as in an siRNA. Such double-stranded nucleic acids may have a short nucleotide overhang at one or both ends of the duplex structure. Double-stranded nucleic acids also include structures formed from a single oligonucleotide with sufficient length and self-complementarity to form a duplex structure, as in an shRNA. Such double-stranded nucleic acids include stem-loop structures. A double-stranded nucleic acid may include one or more modifications relative to a naturally occurring terminus, sugar, nucleobase, and/or phosphate group.

“Double-stranded region” means the portion of a double-stranded nucleic acid where nucleotides of the first nucleotide sequence are hybridized to nucleotides of the second nucleotide sequence. A double-stranded region can be a defined portion within a double-stranded nucleic acid that is shorter than (e.g. encompassed by) the full double-stranded nucleic acid. Alternatively, a double-stranded region can be the same length as the full double-stranded nucleic acid. A double-stranded region may contain one or more mismatches between the first and second nucleotide sequences, and retain the ability hybridize with each other. Double-stranded regions do not include nucleotide overhangs.

“Antisense strand” means an oligonucleotide that is complementary to a target RNA (e.g. a mRNA) and is incorporated into the RNA-induced silencing complex (RISC) to direct gene silencing in a sequence-specific manner through the RNA interference pathway. The antisense strand may also be referred to as the “guide strand.”

“Sense strand” means an oligonucleotide that is complementary to the antisense strand of a double-stranded nucleic acid. The sense strand is typically degraded following incorporation of the antisense strand into RISC. The sense strand may also be referred to as the “passenger strand.”

“Nucleotide overhang” means an extension of one or more unpaired nucleotides from the double-stranded region of a double-stranded nucleic acid. For example, when the 3′ terminus of an antisense strand extends beyond the 5′ terminus of a sense strand, the 3′ terminus of the antisense strand has a nucleotide overhang. A nucleotide overhang can be one, two, three, four or five nucleotides. One or more nucleotides of a nucleotide overhang may be a modified nucleotide. A nucleotide overhang may be on the antisense strand, the sense strand, or both the antisense and sense strands.

“Blunt end” means a given terminus of a double-stranded nucleic acid with no unpaired nucleotides extending from the double-stranded region, i.e. there is no nucleotide overhang. A double-stranded nucleic acid may have a blunt end at one or both termini.

“siRNA” means a double-stranded nucleic acid formed from separate antisense and sense strands, which directs gene silencing in a sequence-specific manner by facilitating mRNA degradation before translation through the RNA interference pathway. The antisense and sense strands of an siRNA are not covalently linked.

“shRNA” means a double-stranded nucleic acid containing a loop structure that is processed in a cell to an siRNA which directs gene silencing in a sequence-specific manner, by facilitating mRNA degradation before translation through the RNA interference pathway.

“Single-stranded nucleic acid” means an antisense strand that is not hybridized to a complementary strand. A single-stranded nucleic acid is incorporated into RISC to direct gene silencing in a sequence-specific manner by facilitating mRNA degradation before translation through the RNA interference pathway.

“Hybridize” means the annealing of one nucleotide sequence to another nucleotide sequence based at least in part on nucleotide sequence complementarity. In embodiments, an antisense strand is hybridized to a sense strand. In embodiments, an antisense strand hybridizes to a target mRNA sequence.

“Complementary” means nucleobases having the capacity to pair non-covalently via hydrogen bonding.

“Fully complementary” or “100% complementary” means each nucleobase of a first nucleotide sequence is complementary to each nucleobase of a second nucleotide sequence. In embodiments, an antisense strand is fully complementary to its target mRNA. In embodiments, a sense strand and an antisense strand of double-stranded nucleic acid are fully complementary over their entire lengths. In embodiments, a sense strand and an antisense strand of double-stranded nucleic acid are fully complementary over the entire length of the double-stranded region of the siRNA, and one or both termini of either strand comprises single-stranded nucleotides.

“Percent complementary” means the percentage of nucleobases of an oligonucleotide that are complementary to an equal-length portion of a target nucleic acid. Percent complementarity is calculated by dividing the number of nucleobases of the oligonucleotide that are complementary to nucleobases at corresponding positions in the target nucleic acid by the total number of nucleobases in the oligonucleotide.

“Identical” in the context of nucleotide sequences, means having the same nucleotide sequence, independent of sugar, linkage, and/or nucleobase modifications and independent of the methylation state of any pyrimidines present.

“Percent identity” means the number of nucleobases in a first nucleotide sequence that are identical to nucleobases at corresponding positions in a second nucleotide sequence, divided by the total number of nucleobases in the first nucleotide sequence.

“Mismatch” means a nucleobase of a first nucleotide sequence that is not capable of Watson-Crick pairing with a nucleobase at a corresponding position of a second nucleotide sequence.

“Nucleoside” means a monomer of a nucleobase and a pentofuranosyl sugar (e.g., either ribose or deoxyribose). Nucleosides may comprise bases such as A, C, G, T, or U, or modifications thereof. Nucleosides may be modified at the base and/or and the sugar. In embodiments, a nucleoside is a deoxyribonucleoside. In embodiments, the nucleoside is a ribonucleoside.

“Nucleotide” means a nucleoside covalently linked to a phosphate group at the 5′ carbon of the pentafuranosyl sugar. Nucleotides may be modified at one or more of the nucleobase, sugar moiety, internucleotide linkage and/or phosphate group.

“Nucleobase” means a heterocyclic base moiety capable of non-covalently pairing. Nucleobases include pyrimidines and purines. Unless stated otherwise, conventional nucleobase abbreviations are used herein. Nucleobases abbreviations include, without limitation, A (adenine), C (cytosine), G (guanine), T (thymine), U (uracil).

Unless stated otherwise, numbering of nucleotide atoms is according to standard numbering convention, with the carbons of the pentafuranosyl sugar numbered 1′ through 5′, and the nucleobase atoms numbered 1 through 9 for purines and 1 through 6 for pyrimidines.

“Modified nucleoside” means a nucleoside having one or more modifications relative to a naturally occurring nucleoside. Such alterations may be present in a nucleobase and/or sugar moiety of the nucleoside. A modified nucleoside may have a modified sugar moiety and an unmodified nucleobase. A modified nucleoside may have a modified sugar moiety and a modified nucleobase.

“Modified nucleotide” means a nucleotide having one or more alterations relative to a naturally occurring nucleotide. An alteration may be present in an internucleoside linkage, a nucleobase, and/or a sugar moiety of the nucleotide. A modified nucleotide may have a modified sugar moiety and an unmodified phosphate group. A modified nucleotide may have an unmodified sugar moiety and a modified phosphate group. A modified nucleotide may have a modified sugar moiety and an unmodified nucleobase. A modified nucleotide may have a modified sugar moiety and a modified phosphate group.

“Modified nucleobase” means a nucleobase having one or more alterations relative to a naturally occurring nucleobase.

“Modified phosphate group” means any change from a naturally occurring phosphate group of a nucleotide.

“Modified internucleotide linkage” means any change from a naturally occurring phosphodiester linkage between two nucleotides.

“Phosphorothioate internucleotide linkage” means a substituted phosphodiester internucleotide linkage where one of the non-bridging atoms is a sulfur atom.

“Modified sugar moiety” means a sugar of a nucleotide having any change and/or substitution from a naturally occurring sugar moiety.

“beta-D-deoxyribonucleoside” means a naturally occurring nucleoside monomer of DNA.

“beta-D-ribonucleoside” means a naturally occurring nucleoside monomer of RNA.

“2′-O-methyl sugar” or “2′-OMe sugar” means a sugar having an O—CH₃ substitution at the 2′ position of the pentofuranosyl sugar.

“2′-O-methoxyethyl sugar” or “2′-MOE sugar” means a sugar having an OCH₂CH₂OCH₃ substitution at the 2′ position of the pentofuranosyl sugar.

“2-fluoro sugar” or “2′-F sugar” means a sugar having a fluoro substitution at the 2′ position of the pentofuranosyl sugar.

“Bicyclic sugar” means a modified sugar moiety comprising a linkage connecting the 2′-carbon and 4′-carbon of the pentafuranosyl sugar, resulting in a bicyclic structure. Nonlimiting exemplary bicyclic sugar moieties include LNA, ENA, cEt, S-cEt, and R-cEt.

“Locked nucleic acid (LNA) sugar” means a substituted sugar moiety comprising a —CH₂—O— linkage between the 4′ and 2′ furanose ring atoms.

“ENA sugar” means a substituted sugar moiety comprising a —(CH₂)₂-O— linkage between the 4′ and 2′ furanose ring atoms.

“2′-O-methyl nucleotide” means a nucleotide having an O-methyl substitution at the 2′ position of the pentofuranosyl sugar. A 2′-O-methyl nucleotide may have a further modification in addition to the modified sugar moiety, for example a modified nucleobase and/or phosphate group.

“2′-fluoro nucleotide” means a nucleotide having a fluoro substitution at the 2′ position of the pentofuranosyl sugar. A 2′-O-fluoro nucleotide may have a further modification in addition to the modified sugar moiety, for example a modified nucleobase and/or phosphate group.

“Bicyclic nucleotide” means a nucleotide having a linkage connecting the 2′-carbon and 4′-carbon of the pentafuranosyl sugar. A bicyclic nucleotide may have a further modification in addition to the modified sugar moiety, for example a modified nucleobase and/or phosphate group.

“5′-(E)-vinylphosphonate” or “5′-VP”, refers to a chemical moiety having the structure:

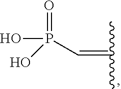 [SMILES: *=CP(=O)(O)O]

or salts thereof, where the wavy line represent the point of attachment to the 5′ carbon of the pentafuranosyl sugar of a nucleotide.

“5-methylcytosine” means a cytosine nucleobase having a 5-methyl substitution on the cytosine ring.

“Non-methylated cytosine” means a cytosine nucleobase that does not have a methyl substitution at the 5 position of the cytosine ring.

“5-methyluracil” means a uracil nucleobase having a 5-methyl substitution on the uracil ring. A 5-methyluracil nucleobase may also be referred to as a thymine.

“Non-methylated uracil” means a uracil nucleobase that does not have a methyl group substitution at the 5 position of the uracil ring.

The term “alkyl,” by itself or as part of another substituent, means, unless otherwise stated, a straight (i.e., unbranched) or branched carbon chain (or carbon), or combination thereof, which may be fully saturated, mono- or polyunsaturated and can include mono-, di- and multivalent radicals. The alkyl may include a designated number of carbons (e.g., C₁-C₁₀ means one to ten carbons). Alkyl is an uncyclized chain. Examples of saturated hydrocarbon radicals include, but are not limited to, groups such as methyl, ethyl, n-propyl, isopropyl, n-butyl, t-butyl, isobutyl, sec-butyl, methyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. An alkoxy is an alkyl attached to the remainder of the molecule via an oxygen linker (—O—). An alkyl moiety may be an alkenyl moiety. An alkyl moiety may be an alkynyl moiety. An alkyl moiety may be fully saturated. An alkenyl may include more than one double bond and/or one or more triple bonds in addition to the one or more double bonds. An alkynyl may include more than one triple bond and/or one or more double bonds in addition to the one or more triple bonds.

The term “cycloalkyl” means a monocyclic, bicyclic, or a multicyclic cycloalkyl ring system. In embodiments, monocyclic ring systems are cyclic hydrocarbon groups containing from 3 to 8 carbon atoms, where such groups can be saturated or unsaturated, but not aromatic. In embodiments, cycloalkyl groups are fully saturated. Examples of monocyclic cycloalkyls include cyclopropyl, cyclobutyl, cyclopentyl, cyclopentenyl, cyclohexyl, cyclohexenyl, cycloheptyl, and cyclooctyl. Bicyclic cycloalkyl ring systems are bridged monocyclic rings or fused bicyclic rings. In embodiments, bridged monocyclic rings contain a monocyclic cycloalkyl ring where two non adjacent carbon atoms of the monocyclic ring are linked by an alkylene bridge of between one and three additional carbon atoms (i.e., a bridging group of the form (CH₂)_{w}, where w is 1, 2, or 3). Representative examples of bicyclic ring systems include, but are not limited to, bicyclo[3.1.1]heptane, bicyclo[2.2.1]heptane, bicyclo[2.2.2]octane, bicyclo[3.2.2]nonane, bicyclo[3.3.1]nonane, and bicyclo[4.2.1]nonane. In embodiments, fused bicyclic cycloalkyl ring systems contain a monocyclic cycloalkyl ring fused to either a phenyl, a monocyclic cycloalkyl, a monocyclic cycloalkenyl, a monocyclic heterocyclyl, or a monocyclic heteroaryl. In embodiments, the bridged or fused bicyclic cycloalkyl is attached to the parent molecular moiety through any carbon atom contained within the monocyclic cycloalkyl ring. In embodiments, cycloalkyl groups are optionally substituted with one or two groups which are independently oxo or thia. In embodiments, the fused bicyclic cycloalkyl is a 5 or 6 membered monocyclic cycloalkyl ring fused to either a phenyl ring, a 5 or 6 membered monocyclic cycloalkyl, a 5 or 6 membered monocyclic cycloalkenyl, a 5 or 6 membered monocyclic heterocyclyl, or a 5 or 6 membered monocyclic heteroaryl, wherein the fused bicyclic cycloalkyl is optionally substituted by one or two groups which are independently oxo or thia. In embodiments, multicyclic cycloalkyl ring systems are a monocyclic cycloalkyl ring (base ring) fused to either (i) one ring system selected from the group consisting of a bicyclic aryl, a bicyclic heteroaryl, a bicyclic cycloalkyl, a bicyclic cycloalkenyl, and a bicyclic heterocyclyl; or (ii) two other ring systems independently selected from the group consisting of a phenyl, a bicyclic aryl, a monocyclic or bicyclic heteroaryl, a monocyclic or bicyclic cycloalkyl, a monocyclic or bicyclic cycloalkenyl, and a monocyclic or bicyclic heterocyclyl. In embodiments, the multicyclic cycloalkyl is attached to the parent molecular moiety through any carbon atom contained within the base ring. In embodiments, multicyclic cycloalkyl ring systems are a monocyclic cycloalkyl ring (base ring) fused to either (i) one ring system selected from the group consisting of a bicyclic aryl, a bicyclic heteroaryl, a bicyclic cycloalkyl, a bicyclic cycloalkenyl, and a bicyclic heterocyclyl; or (ii) two other ring systems independently selected from the group consisting of a phenyl, a monocyclic heteroaryl, a monocyclic cycloalkyl, a monocyclic cycloalkenyl, and a monocyclic heterocyclyl. Examples of multicyclic cycloalkyl groups include, but are not limited to tetradecahydrophenanthrenyl, perhydrophenothiazin-1-yl, and perhydrophenoxazin-1-yl.

In embodiments, a cycloalkyl is a cycloalkenyl. The term “cycloalkenyl” is used in accordance with its plain ordinary meaning. In embodiments, a cycloalkenyl is a monocyclic, bicyclic, or a multicyclic cycloalkenyl ring system. In embodiments, monocyclic cycloalkenyl ring systems are cyclic hydrocarbon groups containing from 3 to 8 carbon atoms, where such groups are unsaturated (i.e., containing at least one annular carbon carbon double bond), but not aromatic. Examples of monocyclic cycloalkenyl ring systems include cyclopentenyl and cyclohexenyl. In embodiments, bicyclic cycloalkenyl rings are bridged monocyclic rings or a fused bicyclic rings. In embodiments, bridged monocyclic rings contain a monocyclic cycloalkenyl ring where two non adjacent carbon atoms of the monocyclic ring are linked by an alkylene bridge of between one and three additional carbon atoms (i.e., a bridging group of the form (CH₂)_{w}, where w is 1, 2, or 3). Representative examples of bicyclic cycloalkenyls include, but are not limited to, norbornenyl and bicyclo[2.2.2]oct 2 enyl. In embodiments, fused bicyclic cycloalkenyl ring systems contain a monocyclic cycloalkenyl ring fused to either a phenyl, a monocyclic cycloalkyl, a monocyclic cycloalkenyl, a monocyclic heterocyclyl, or a monocyclic heteroaryl. In embodiments, the bridged or fused bicyclic cycloalkenyl is attached to the parent molecular moiety through any carbon atom contained within the monocyclic cycloalkenyl ring. In embodiments, cycloalkenyl groups are optionally substituted with one or two groups which are independently oxo or thia. In embodiments, multicyclic cycloalkenyl rings contain a monocyclic cycloalkenyl ring (base ring) fused to either (i) one ring system selected from the group consisting of a bicyclic aryl, a bicyclic heteroaryl, a bicyclic cycloalkyl, a bicyclic cycloalkenyl, and a bicyclic heterocyclyl; or (ii) two ring systems independently selected from the group consisting of a phenyl, a bicyclic aryl, a monocyclic or bicyclic heteroaryl, a monocyclic or bicyclic cycloalkyl, a monocyclic or bicyclic cycloalkenyl, and a monocyclic or bicyclic heterocyclyl. In embodiments, the multicyclic cycloalkenyl is attached to the parent molecular moiety through any carbon atom contained within the base ring. In embodiments, multicyclic cycloalkenyl rings contain a monocyclic cycloalkenyl ring (base ring) fused to either (i) one ring system selected from the group consisting of a bicyclic aryl, a bicyclic heteroaryl, a bicyclic cycloalkyl, a bicyclic cycloalkenyl, and a bicyclic heterocyclyl; or (ii) two ring systems independently selected from the group consisting of a phenyl, a monocyclic heteroaryl, a monocyclic cycloalkyl, a monocyclic cycloalkenyl, and a monocyclic heterocyclyl.

In embodiments, a heterocycloalkyl is a heterocyclyl. The term “heterocyclyl” as used herein, means a monocyclic, bicyclic, or multicyclic heterocycle. The heterocyclyl monocyclic heterocycle is a 3, 4, 5, 6 or 7 membered ring containing at least one heteroatom independently selected from the group consisting of O, N, and S where the ring is saturated or unsaturated, but not aromatic. The 3 or 4 membered ring contains 1 heteroatom selected from the group consisting of O, N and S. The 5 membered ring can contain zero or one double bond and one, two or three heteroatoms selected from the group consisting of O, N and S. The 6 or 7 membered ring contains zero, one or two double bonds and one, two or three heteroatoms selected from the group consisting of O, N and S. The heterocyclyl monocyclic heterocycle is connected to the parent molecular moiety through any carbon atom or any nitrogen atom contained within the heterocyclyl monocyclic heterocycle. Representative examples of heterocyclyl monocyclic heterocycles include, but are not limited to, azetidinyl, azepanyl, aziridinyl, diazepanyl, 1,3 dioxanyl, 1,3 dioxolanyl, 1,3 dithiolanyl, 1,3 dithianyl, imidazolinyl, imidazolidinyl, isothiazolinyl, isothiazolidinyl, isoxazolinyl, isoxazolidinyl, morpholinyl, oxadiazolinyl, oxadiazolidinyl, oxazolinyl, oxazolidinyl, piperazinyl, piperidinyl, pyranyl, pyrazolinyl, pyrazolidinyl, pyrrolinyl, pyrrolidinyl, tetrahydrofuranyl, tetrahydrothienyl, thiadiazolinyl, thiadiazolidinyl, thiazolinyl, thiazolidinyl, thiomorpholinyl, 1,1 dioxidothiomorpholinyl (thiomorpholine sulfone), thiopyranyl, and trithianyl. The heterocyclyl bicyclic heterocycle is a monocyclic heterocycle fused to either a phenyl, a monocyclic cycloalkyl, a monocyclic cycloalkenyl, a monocyclic heterocycle, or a monocyclic heteroaryl. The heterocyclyl bicyclic heterocycle is connected to the parent molecular moiety through any carbon atom or any nitrogen atom contained within the monocyclic heterocycle portion of the bicyclic ring system. Representative examples of bicyclic heterocyclyls include, but are not limited to, 2,3 dihydrobenzofuran 2 yl, 2,3 dihydrobenzofuran 3 yl, indolin 1 yl, indolin 2 yl, indolin 3 yl, 2,3 dihydrobenzothien 2 yl, decahydroquinolinyl, decahydroisoquinolinyl, octahydro 1H indolyl, and octahydrobenzofuranyl. In embodiments, heterocyclyl groups are optionally substituted with one or two groups which are independently oxo or thia. In certain embodiments, the bicyclic heterocyclyl is a 5 or 6 membered monocyclic heterocyclyl ring fused to a phenyl ring, a 5 or 6 membered monocyclic cycloalkyl, a 5 or 6 membered monocyclic cycloalkenyl, a 5 or 6 membered monocyclic heterocyclyl, or a 5 or 6 membered monocyclic heteroaryl, wherein the bicyclic heterocyclyl is optionally substituted by one or two groups which are independently oxo or thia. Multicyclic heterocyclyl ring systems are a monocyclic heterocyclyl ring (base ring) fused to either (i) one ring system selected from the group consisting of a bicyclic aryl, a bicyclic heteroaryl, a bicyclic cycloalkyl, a bicyclic cycloalkenyl, and a bicyclic heterocyclyl; or (ii) two other ring systems independently selected from the group consisting of a phenyl, a bicyclic aryl, a monocyclic or bicyclic heteroaryl, a monocyclic or bicyclic cycloalkyl, a monocyclic or bicyclic cycloalkenyl, and a monocyclic or bicyclic heterocyclyl. The multicyclic heterocyclyl is attached to the parent molecular moiety through any carbon atom or nitrogen atom contained within the base ring. In embodiments, multicyclic heterocyclyl ring systems are a monocyclic heterocyclyl ring (base ring) fused to either (i) one ring system selected from the group consisting of a bicyclic aryl, a bicyclic heteroaryl, a bicyclic cycloalkyl, a bicyclic cycloalkenyl, and a bicyclic heterocyclyl; or (ii) two other ring systems independently selected from the group consisting of a phenyl, a monocyclic heteroaryl, a monocyclic cycloalkyl, a monocyclic cycloalkenyl, and a monocyclic heterocyclyl. Examples of multicyclic heterocyclyl groups include, but are not limited to 10H-phenothiazin-10-yl, 9,10-dihydroacridin-9-yl, 9,10-dihydroacridin-10-yl, 10H-phenoxazin-10-yl, 10,11-dihydro-5H-dibenzo[b,f]azepin-5-yl, 1,2,3,4-tetrahydropyrido[4,3-g]isoquinolin-2-yl, 12H-benzo[b]phenoxazin-12-yl, and dodecahydro-1H-carbazol-9-yl.

The term “alkylene,” by itself or as part of another substituent, means, unless otherwise stated, a divalent radical derived from an alkyl, as exemplified, but not limited by, —CH₂CH₂CH₂CH₂—. Typically, an alkyl (or alkylene) group will have from 1 to 24 carbon atoms, with those groups having 10 or fewer carbon atoms being preferred herein. A “lower alkyl” or “lower alkylene” is a shorter chain alkyl or alkylene group, generally having eight or fewer carbon atoms. The term “alkenylene,” by itself or as part of another substituent, means, unless otherwise stated, a divalent radical derived from an alkene.

The term “heteroalkyl,” by itself or in combination with another term, means, unless otherwise stated, a stable straight or branched chain, or combinations thereof, including at least one carbon atom and at least one heteroatom (e.g., O, N, S, Si, or P), and wherein the nitrogen and sulfur atoms may optionally be oxidized, and the nitrogen heteroatom may optionally be quaternized. The heteroatom(s) (e.g., O, N, S, Si, or P) may be placed at any interior position of the heteroalkyl group or at the position at which the alkyl group is attached to the remainder of the molecule. Heteroalkyl is an uncyclized chain. Examples include, but are not limited to: —CH₂—CH₂—O—CH₃, —CH₂—CH₂—NH—CH₃, —CH₂—CH₂—N(CH₃)—CH₃, —CH₂—S—CH₂—CH₃, —CH₂—CH₂, —S(O)—CH₃, —CH₂—CH₂—S(O)₂—CH₃, —CH═CH—O—CH₃, —Si(CH₃)₃, —CH₂—CH═N—OCH₃, —CH═CH—N(CH₃)—CH₃, —O—CH₃, —O—CH₂—CH₃, and —CN. Up to two or three heteroatoms may be consecutive, such as, for example, —CH₂—NH—OCH₃ and —CH₂—O—Si(CH₃)₃. A heteroalkyl moiety may include one heteroatom (e.g., O, N, S, Si, or P). A heteroalkyl moiety may include two optionally different heteroatoms (e.g., O, N, S, Si, or P). A heteroalkyl moiety may include three optionally different heteroatoms (e.g., O, N, S, Si, or P). A heteroalkyl moiety may include four optionally different heteroatoms (e.g., O, N, S, Si, or P). A heteroalkyl moiety may include five optionally different heteroatoms (e.g., O, N, S, Si, or P). A heteroalkyl moiety may include up to 8 optionally different heteroatoms (e.g., O, N, S, Si, or P). The term “heteroalkenyl,” by itself or in combination with another term, means, unless otherwise stated, a heteroalkyl including at least one double bond. A heteroalkenyl may optionally include more than one double bond and/or one or more triple bonds in additional to the one or more double bonds. The term “heteroalkynyl,” by itself or in combination with another term, means, unless otherwise stated, a heteroalkyl including at least one triple bond. A heteroalkynyl may optionally include more than one triple bond and/or one or more double bonds in additional to the one or more triple bonds.

Similarly, the term “heteroalkylene,” by itself or as part of another substituent, means, unless otherwise stated, a divalent radical derived from heteroalkyl, as exemplified, but not limited by, —CH₂—CH₂—S—CH₂—CH₂— and —CH₂—S—CH₂—CH₂—NH—CH₂—. For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. For example, the formula —C(O)₂R′— represents both —C(O)₂R′- and —R′C(O)₂—. As described above, heteroalkyl groups, as used herein, include those groups that are attached to the remainder of the molecule through a heteroatom, such as —C(O)R′, —C(O)NR′, —NR′R″, —OR′, —SR′, and/or —SO₂R′. Where “heteroalkyl” is recited, followed by recitations of specific heteroalkyl groups, such as —NR′R″ or the like, it will be understood that the terms heteroalkyl and —NR′R″ are not redundant or mutually exclusive. Rather, the specific heteroalkyl groups are recited to add clarity. Thus, the term “heteroalkyl” should not be interpreted herein as excluding specific heteroalkyl groups, such as —NR′R″ or the like.

The terms “cycloalkyl” and “heterocycloalkyl,” by themselves or in combination with other terms, mean, unless otherwise stated, cyclic versions of “alkyl” and “heteroalkyl,” respectively. Cycloalkyl and heterocycloalkyl are not aromatic. Additionally, for heterocycloalkyl, a heteroatom can occupy the position at which the heterocycle is attached to the remainder of the molecule. Examples of cycloalkyl include, but are not limited to, cyclopropyl, cyclobutyl, cyclopentyl, cyclohexyl, 1-cyclohexenyl, 3-cyclohexenyl, cycloheptyl, and the like. Examples of heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like. A “cycloalkylene” and a “heterocycloalkylene,” alone or as part of another substituent, means a divalent radical derived from a cycloalkyl and heterocycloalkyl, respectively.

The terms “halo” or “halogen,” by themselves or as part of another substituent, mean, unless otherwise stated, a fluorine, chlorine, bromine, or iodine atom. Additionally, terms such as “haloalkyl” are meant to include monohaloalkyl and polyhaloalkyl. For example, the term “halo(C₁-C₄)alkyl” includes, but is not limited to, fluoromethyl, difluoromethyl, trifluoromethyl, 2,2,2-trifluoroethyl, 4-chlorobutyl, 3-bromopropyl, and the like.

The term “acyl” means, unless otherwise stated, —C(O)R where R is a substituted or unsubstituted alkyl, substituted or unsubstituted cycloalkyl, substituted or unsubstituted heteroalkyl, substituted or unsubstituted heterocycloalkyl, substituted or unsubstituted aryl, or substituted or unsubstituted heteroaryl.

The term “aryl” means, unless otherwise stated, a polyunsaturated, aromatic, hydrocarbon substituent, which can be a single ring or multiple rings (preferably from 1 to 3 rings) that are fused together (i.e., a fused ring aryl) or linked covalently. A fused ring aryl refers to multiple rings fused together wherein at least one of the fused rings is an aryl ring. The term “heteroaryl” refers to aryl groups (or rings) that contain at least one heteroatom such as N, O, or S, wherein the nitrogen and sulfur atoms are optionally oxidized, and the nitrogen atom(s) are optionally quaternized. Thus, the term “heteroaryl” includes fused ring heteroaryl groups (i.e., multiple rings fused together wherein at least one of the fused rings is a heteroaromatic ring). A 5,6-fused ring heteroarylene refers to two rings fused together, wherein one ring has 5 members and the other ring has 6 members, and wherein at least one ring is a heteroaryl ring. Likewise, a 6,6-fused ring heteroarylene refers to two rings fused together, wherein one ring has 6 members and the other ring has 6 members, and wherein at least one ring is a heteroaryl ring. And a 6,5-fused ring heteroarylene refers to two rings fused together, wherein one ring has 6 members and the other ring has 5 members, and wherein at least one ring is a heteroaryl ring. A heteroaryl group can be attached to the remainder of the molecule through a carbon or heteroatom. Non-limiting examples of aryl and heteroaryl groups include phenyl, naphthyl, pyrrolyl, pyrazolyl, pyridazinyl, triazinyl, pyrimidinyl, imidazolyl, pyrazinyl, purinyl, oxazolyl, isoxazolyl, thiazolyl, furyl, thienyl, pyridyl, pyrimidyl, benzothiazolyl, benzoxazoyl benzimidazolyl, benzofuran, isobenzofuranyl, indolyl, isoindolyl, benzothiophenyl, isoquinolyl, quinoxalinyl, quinolyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of the above noted aryl and heteroaryl ring systems are selected from the group of acceptable substituents described below. An “arylene” and a “heteroarylene,” alone or as part of another substituent, mean a divalent radical derived from an aryl and heteroaryl, respectively. A heteroaryl group substituent may be —O— bonded to a ring heteroatom nitrogen.

Spirocyclic rings are two or more rings wherein adjacent rings are attached through a single atom. The individual rings within spirocyclic rings may be identical or different. Individual rings in spirocyclic rings may be substituted or unsubstituted and may have different substituents from other individual rings within a set of spirocyclic rings. Possible substituents for individual rings within spirocyclic rings are the possible substituents for the same ring when not part of spirocyclic rings (e.g. substituents for cycloalkyl or heterocycloalkyl rings). Spirocylic rings may be substituted or unsubstituted cycloalkyl, substituted or unsubstituted cycloalkylene, substituted or unsubstituted heterocycloalkyl or substituted or unsubstituted heterocycloalkylene and individual rings within a spirocyclic ring group may be any of the immediately previous list, including having all rings of one type (e.g. all rings being substituted heterocycloalkylene wherein each ring may be the same or different substituted heterocycloalkylene). When referring to a spirocyclic ring system, heterocyclic spirocyclic rings means a spirocyclic rings wherein at least one ring is a heterocyclic ring and wherein each ring may be a different ring. When referring to a spirocyclic ring system, substituted spirocyclic rings means that at least one ring is substituted and each substituent may optionally be different.

The symbol “ [symbol] ” denotes the point of attachment of a chemical moiety to the remainder of a molecule or chemical formula.

The term “oxo,” as used herein, means an oxygen that is double bonded to a carbon atom.

The term “alkylarylene” as an arylene moiety covalently bonded to an alkylene moiety (also referred to herein as an alkylene linker). In embodiments, the alkylarylene group has the formula:

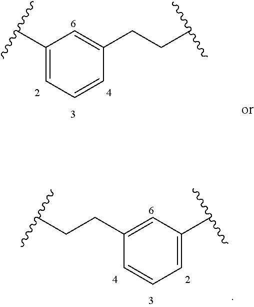 [SMILES: *CCc1cccc(*)c1 ; *CCc1cccc(*)c1]

An alkylarylene moiety may be substituted (e.g. with a substituent group) on the alkylene moiety or the arylene linker (e.g. at carbons 2, 3, 4, or 6) with halogen, oxo, —N₃, —CF₃, —CCl₃, —CBr₃, —CI₃, —CN, —CHO, —OH, —NH₂, —COOH, —CONH₂, —NO₂, —SH, —SO₂CH₃ —SO₃H, —OSO₃H, —SO₂NH₂, —NHNH₂, —ONH₂, —NHC(O)NHNH₂, substituted or unsubstituted C₁-C₅ alkyl or substituted or unsubstituted 2 to 5 membered heteroalkyl). In embodiments, the alkylarylene is unsubstituted.

Each of the above terms (e.g., “alkyl,” “heteroalkyl,” “cycloalkyl,” “heterocycloalkyl,” “aryl,” and “heteroaryl”) includes both substituted and unsubstituted forms of the indicated radical. Preferred substituents for each type of radical are provided below.

Substituents for the alkyl and heteroalkyl radicals (including those groups often referred to as alkylene, alkenyl, heteroalkylene, heteroalkenyl, alkynyl, cycloalkyl, heterocycloalkyl, cycloalkenyl, and heterocycloalkenyl) can be one or more of a variety of groups selected from, but not limited to, —OR′, ═O, ═NR′, =N—OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)═NR″″, —NR—C(NR′R″)=NR′″, —S(O)R′, —S(O)₂R′, —S(O)₂NR′R″, —NRSO₂R′, —NR′NR″R′″, —ONR′R″, —NR′C(O)NR″NR′″R″″, —CN, —NO₂, —NR′SO₂R″, —NR′C(O)R″, —NR′C(O)—OR″, —NR′OR″, in a number ranging from zero to (2m′+1), where m′ is the total number of carbon atoms in such radical. R, R′, R″, R′″, and R″″ each preferably independently refer to hydrogen, substituted or unsubstituted heteroalkyl, substituted or unsubstituted cycloalkyl, substituted or unsubstituted heterocycloalkyl, substituted or unsubstituted aryl (e.g., aryl substituted with 1-3 halogens), substituted or unsubstituted heteroaryl, substituted or unsubstituted alkyl, alkoxy, or thioalkoxy groups, or arylalkyl groups. When a compound described herein includes more than one R group, for example, each of the R groups is independently selected as are each R′, R″, R′″, and R″″ group when more than one of these groups is present. When R′ and R″ are attached to the same nitrogen atom, they can be combined with the nitrogen atom to form a 4-, 5-, 6-, or 7-membered ring. For example, —NR′R″ includes, but is not limited to, 1-pyrrolidinyl and 4-morpholinyl. From the above discussion of substituents, one of skill in the art will understand that the term “alkyl” is meant to include groups including carbon atoms bound to groups other than hydrogen groups, such as haloalkyl (e.g., —CF₃ and —CH₂CF₃) and acyl (e.g., —C(O)CH₃, —C(O)CF₃, —C(O)CH₂OCH₃, and the like).

Similar to the substituents described for the alkyl radical, substituents for the aryl and heteroaryl groups are varied and are selected from, for example: —OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)═NR″″, —NR—C(NR′R″)=NR′″, —S(O)R′, —S(O)₂R′, —S(O)₂NR′R″, —NRSO₂R′, —NR′NR″R′″, —ONR′R″, —NR′C(O)NR″NR′″R″″, —CN, —NO₂, —R′, —N₃, —CH(Ph)₂, fluoro(C₁-C₄)alkoxy, and fluoro(C₁-C₄)alkyl, —NR′SO₂R″, —NR′C(O)R″, —NR′C(O)—OR″, —NR′OR″, in a number ranging from zero to the total number of open valences on the aromatic ring system; and where R′, R″, R′″, and R″″ are preferably independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl. When a compound described herein includes more than one R group, for example, each of the R groups is independently selected as are each R′, R″, R′″, and R″″ groups when more than one of these groups is present.

Substituents for rings (e.g. cycloalkyl, heterocycloalkyl, aryl, heteroaryl, cycloalkylene, heterocycloalkylene, arylene, or heteroarylene) may be depicted as substituents on the ring rather than on a specific atom of a ring (commonly referred to as a floating substituent). In such a case, the substituent may be attached to any of the ring atoms (obeying the rules of chemical valency) and in the case of fused rings or spirocyclic rings, a substituent depicted as associated with one member of the fused rings or spirocyclic rings (a floating substituent on a single ring), may be a substituent on any of the fused rings or spirocyclic rings (a floating substituent on multiple rings). When a substituent is attached to a ring, but not a specific atom (a floating substituent), and a subscript for the substituent is an integer greater than one, the multiple substituents may be on the same atom, same ring, different atoms, different fused rings, different spirocyclic rings, and each substituent may optionally be different. Where a point of attachment of a ring to the remainder of a molecule is not limited to a single atom (a floating substituent), the attachment point may be any atom of the ring and in the case of a fused ring or spirocyclic ring, any atom of any of the fused rings or spirocyclic rings while obeying the rules of chemical valency. Where a ring, fused rings, or spirocyclic rings contain one or more ring heteroatoms and the ring, fused rings, or spirocyclic rings are shown with one more floating substituents (including, but not limited to, points of attachment to the remainder of the molecule), the floating substituents may be bonded to the heteroatoms. Where the ring heteroatoms are shown bound to one or more hydrogens (e.g. a ring nitrogen with two bonds to ring atoms and a third bond to a hydrogen) in the structure or formula with the floating substituent, when the heteroatom is bonded to the floating substituent, the substituent will be understood to replace the hydrogen, while obeying the rules of chemical valency.

Two or more substituents may optionally be joined to form aryl, heteroaryl, cycloalkyl, or heterocycloalkyl groups. Such so-called ring-forming substituents are typically, though not necessarily, found attached to a cyclic base structure. In one embodiment, the ring-forming substituents are attached to adjacent members of the base structure. For example, two ring-forming substituents attached to adjacent members of a cyclic base structure create a fused ring structure. In another embodiment, the ring-forming substituents are attached to a single member of the base structure. For example, two ring-forming substituents attached to a single member of a cyclic base structure create a spirocyclic structure. In yet another embodiment, the ring-forming substituents are attached to non-adjacent members of the base structure.

Two of the substituents on adjacent atoms of the aryl or heteroaryl ring may optionally form a ring of the formula -T-C(O)—(CRR′)_{q}—U—, wherein T and U are independently —NR—, —O—, —CRR′—, or a single bond, and q is an integer of from 0 to 3. Alternatively, two of the substituents on adjacent atoms of the aryl or heteroaryl ring may optionally be replaced with a substituent of the formula -A-(CH₂)ᵣ—B—, wherein A and B are independently —CRR′—, —O—, —NR—, —S—, —S(O)—, —S(O)₂—, —S(O)₂NR′—, or a single bond, and r is an integer of from 1 to 4. One of the single bonds of the new ring so formed may optionally be replaced with a double bond. Alternatively, two of the substituents on adjacent atoms of the aryl or heteroaryl ring may optionally be replaced with a substituent of the formula —(CRR′)ₛ—X′—(C″R″R′″)_{d}—, where s and d are independently integers of from 0 to 3, and X′ is —O—, —NR′—, —S—, —S(O)—, —S(O)₂—, or —S(O)₂NR′—. The substituents R, R′, R″, and R′″ are preferably independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl.

As used herein, the terms “heteroatom” or “ring heteroatom” are meant to include oxygen (O), nitrogen (N), sulfur (S), phosphorus (P), and silicon (Si).

A “substituent group,” as used herein, means a group selected from the following moieties:
(A) oxo, halogen, —CF₃, —CCl₃, —CBr₃, —Cl₃, —CHF₂, —CHCl₂, —CHBr₂, —CHI₂, —CH₂F, —CH₂Cl, —CH₂Br, —CH₂I, —CN, —N₃, —OH, —NH₂, —COOH, —CONH₂, —NO₂, —SH, —SCH₃, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl), and
(B) alkyl, heteroalkyl, cycloalkyl, heterocycloalkyl, aryl, heteroaryl, substituted with at least one substituent selected from:
   (i) oxo, halogen, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —N₃, —OH, —NH₂, —COOH, —CONH₂, —NO₂, —SH, —SCH₃, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl), and
   (ii) alkyl, heteroalkyl, cycloalkyl, heterocycloalkyl, aryl, heteroaryl, substituted with at least one substituent selected from:
      (a) oxo, halogen, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —N₃, —OH, —NH₂, —COOH, —CONH₂, —NO₂, —SH, —SCH₃, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl), and
      (b) alkyl, heteroalkyl, cycloalkyl, heterocycloalkyl, aryl, heteroaryl, substituted with at least one substituent selected from: oxo, halogen, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —N₃, —OH, —NH₂, —COOH, —CONH₂, —NO₂, —SH, —SCH₃, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl).

A “size-limited substituent” or “size-limited substituent group,” as used herein, means a group selected from all of the substituents described above for a “substituent group,” wherein each substituted or unsubstituted alkyl is a substituted or unsubstituted C₁-C₂₀ alkyl, each substituted or unsubstituted heteroalkyl is a substituted or unsubstituted 2 to 20 membered heteroalkyl, each substituted or unsubstituted cycloalkyl is a substituted or unsubstituted C₃-C₈ cycloalkyl, each substituted or unsubstituted heterocycloalkyl is a substituted or unsubstituted 3 to 8 membered heterocycloalkyl, each substituted or unsubstituted aryl is a substituted or unsubstituted C₆-C₁₀ aryl, and each substituted or unsubstituted heteroaryl is a substituted or unsubstituted 5 to 10 membered heteroaryl.

A “lower substituent” or “lower substituent group,” as used herein, means a group selected from all of the substituents described above for a “substituent group,” wherein each substituted or unsubstituted alkyl is a substituted or unsubstituted C₁-C₈ alkyl, each substituted or unsubstituted heteroalkyl is a substituted or unsubstituted 2 to 8 membered heteroalkyl, each substituted or unsubstituted cycloalkyl is a substituted or unsubstituted C₃-C₇ cycloalkyl, each substituted or unsubstituted heterocycloalkyl is a substituted or unsubstituted 3 to 7 membered heterocycloalkyl, each substituted or unsubstituted aryl is a substituted or unsubstituted C₆-C₁₀ aryl, and each substituted or unsubstituted heteroaryl is a substituted or unsubstituted 5 to 9 membered heteroaryl.

In embodiments, a substituted or unsubstituted moiety (e.g., substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, substituted or unsubstituted alkylene, substituted or unsubstituted heteroalkylene, substituted or unsubstituted cycloalkylene, substituted or unsubstituted heterocycloalkylene, substituted or unsubstituted arylene, and/or substituted or unsubstituted heteroarylene) is unsubstituted (e.g., is an unsubstituted alkyl, unsubstituted heteroalkyl, unsubstituted cycloalkyl, unsubstituted heterocycloalkyl, unsubstituted aryl, unsubstituted heteroaryl, unsubstituted alkylene, unsubstituted heteroalkylene, unsubstituted cycloalkylene, unsubstituted heterocycloalkylene, unsubstituted arylene, and/or unsubstituted heteroarylene, respectively). In embodiments, a substituted or unsubstituted moiety (e.g., substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, substituted or unsubstituted alkylene, substituted or unsubstituted heteroalkylene, substituted or unsubstituted cycloalkylene, substituted or unsubstituted heterocycloalkylene, substituted or unsubstituted arylene, and/or substituted or unsubstituted heteroarylene) is substituted (e.g., is a substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene, respectively).

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one substituent group, wherein if the substituted moiety is substituted with a plurality of substituent groups, each substituent group may optionally be different. In embodiments, if the substituted moiety is substituted with a plurality of substituent groups, each substituent group is different.

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one size-limited substituent group, wherein if the substituted moiety is substituted with a plurality of size-limited substituent groups, each size-limited substituent group may optionally be different. In embodiments, if the substituted moiety is substituted with a plurality of size-limited substituent groups, each size-limited substituent group is different.

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one lower substituent group, wherein if the substituted moiety is substituted with a plurality of lower substituent groups, each lower substituent group may optionally be different. In embodiments, if the substituted moiety is substituted with a plurality of lower substituent groups, each lower substituent group is different.

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one substituent group, size-limited substituent group, or lower substituent group; wherein if the substituted moiety is substituted with a plurality of groups selected from substituent groups, size-limited substituent groups, and lower substituent groups; each substituent group, size-limited substituent group, and/or lower substituent group may optionally be different. In embodiments, if the substituted moiety is substituted with a plurality of groups selected from substituent groups, size-limited substituent groups, and lower substituent groups; each substituent group, size-limited substituent group, and/or lower substituent group is different.

In embodiments of the compounds herein, each substituted or unsubstituted alkyl may be a substituted (e.g., substituted with a substituent group, a size-limited substituent group, or lower substituent group) or unsubstituted C₁-C₂₀ alkyl, each substituted or unsubstituted heteroalkyl is a substituted (e.g., substituted with a substituent group, a size-limited substituent group, or lower substituent group) or unsubstituted 2 to 20 membered heteroalkyl, each substituted or unsubstituted cycloalkyl is a substituted (e.g., substituted with a substituent group, a size-limited substituent group, or lower substituent group) or unsubstituted C₃-C₈ cycloalkyl, each substituted or unsubstituted heterocycloalkyl is a substituted (e.g., substituted with a substituent group, a size-limited substituent group, or lower substituent group) or unsubstituted 3 to 8 membered heterocycloalkyl, each or unsubstituted aryl is a substituted (e.g., substituted with a substituent group, a size-limited substituent group, or lower substituent group) or unsubstituted C₆-C₁₀ aryl, and/or each substituted or unsubstituted heteroaryl is a substituted (e.g., substituted with a substituent group, a size-limited substituent group, or lower substituent group) or unsubstituted 5 to 10 membered heteroaryl. In embodiments herein, each substituted or unsubstituted alkylene is a substituted (e.g., substituted with a substituent group, a size-limited substituent group, or lower substituent group) or unsubstituted C₁-C₂₀ alkylene, each substituted or unsubstituted heteroalkylene is a substituted (e.g., substituted with a substituent group, a size-limited substituent group, or lower substituent group) or unsubstituted 2 to 20 membered heteroalkylene, each substituted or unsubstituted cycloalkylene is a substituted (e.g., substituted with a substituent group, a size-limited substituent group, or lower substituent group) or unsubstituted C₃-C₈ cycloalkylene, each substituted or unsubstituted heterocycloalkylene is a substituted (e.g., substituted with a substituent group, a size-limited substituent group, or lower substituent group) or unsubstituted 3 to 8 membered heterocycloalkylene, each substituted or unsubstituted arylene is a substituted (e.g., substituted with a substituent group, a size-limited substituent group, or lower substituent group) or unsubstituted C₆-C₁₀ arylene, and/or each substituted or unsubstituted heteroarylene is a substituted (e.g., substituted with a substituent group, a size-limited substituent group, or lower substituent group) or unsubstituted 5 to 10 membered heteroarylene.

In embodiments, each substituted or unsubstituted alkyl is a substituted (e.g., substituted with a substituent group, a size-limited substituent group, or lower substituent group) or unsubstituted C₁-C₈ alkyl, each substituted or unsubstituted heteroalkyl is a substituted (e.g., substituted with a substituent group, a size-limited substituent group, or lower substituent group) or unsubstituted 2 to 8 membered heteroalkyl, each substituted or unsubstituted cycloalkyl is a substituted (e.g., substituted with a substituent group, a size-limited substituent group, or lower substituent group) or unsubstituted C₃-C₇ cycloalkyl, each substituted or unsubstituted heterocycloalkyl is a substituted (e.g., substituted with a substituent group, a size-limited substituent group, or lower substituent group) or unsubstituted 3 to 7 membered heterocycloalkyl, each substituted or unsubstituted aryl is a substituted (e.g., substituted with a substituent group, a size-limited substituent group, or lower substituent group) or unsubstituted C₆-C₁₀ aryl, and/or each substituted or unsubstituted heteroaryl is a substituted (e.g., substituted with a substituent group, a size-limited substituent group, or lower substituent group) or unsubstituted 5 to 9 membered heteroaryl. In embodiments, each substituted or unsubstituted alkylene is a substituted (e.g., substituted with a substituent group, a size-limited substituent group, or lower substituent group) or unsubstituted C₁-C₈ alkylene, each substituted or unsubstituted heteroalkylene is a substituted (e.g., substituted with a substituent group, a size-limited substituent group, or lower substituent group) or unsubstituted 2 to 8 membered heteroalkylene, each substituted or unsubstituted cycloalkylene is a substituted (e.g., substituted with a substituent group, a size-limited substituent group, or lower substituent group) or unsubstituted C₃-C₇ cycloalkylene, each substituted or unsubstituted heterocycloalkylene is a substituted or unsubstituted 3 to 7 membered heterocycloalkylene, each substituted or unsubstituted arylene is a substituted (e.g., substituted with a substituent group, a size-limited substituent group, or lower substituent group) or unsubstituted C₆-C₁₀ arylene, and/or each substituted or unsubstituted heteroarylene is a substituted (e.g., substituted with a substituent group, a size-limited substituent group, or lower substituent group) or unsubstituted 5 to 9 membered heteroarylene. In embodiments, the compound is a chemical species set forth in the Examples section, figures, or tables below.

Certain compounds provided herein possess asymmetric carbon atoms (optical or chiral centers) or double bonds; the enantiomers, racemates, diastereomers, tautomers, geometric isomers, stereoisometric forms that may be defined, in terms of absolute stereochemistry, as (R)- or (S)- or, as (D)- or (L)- for amino acids, and individual isomers are encompassed within the scope of the present disclosure. The compounds of provided herein do not include those that are known in art to be too unstable to synthesize and/or isolate. Compounds provided herein include those in racemic and optically pure forms. Optically active (R)- and (S)-, or (D)- and (L)-isomers may be prepared using chiral synthons or chiral reagents, or resolved using conventional techniques. When the compounds described herein contain olefinic bonds or other centers of geometric asymmetry, and unless specified otherwise, it is intended that the compounds include both E and Z geometric isomers.

As used herein, the term “isomers” refers to compounds having the same number and kind of atoms, and hence the same molecular weight, but differing in respect to the structural arrangement or configuration of the atoms.

The term “tautomer,” as used herein, refers to one of two or more structural isomers which exist in equilibrium and which are readily converted from one isomeric form to another.

It will be apparent to one skilled in the art that certain compounds provided herein may exist in tautomeric forms, all such tautomeric forms of the compounds being within the scope of the present disclosure.

Where the compounds disclosed herein have at least one chiral center, they may exist as individual enantiomers and diastereomers or as mixtures of such isomers, including racemates. Separation of the individual isomers or selective synthesis of the individual isomers is accomplished by application of various methods which are well known to practitioners in the art. Unless otherwise indicated, all such isomers and mixtures thereof are included in the scope of the compounds disclosed herein. Unless otherwise stated, structures depicted herein are also meant to include all stereochemical forms of the structure; i.e., the (R) and (S) configurations for each asymmetric center. Therefore, single stereochemical isomers as well as enantiomeric and diastereomeric mixtures of the present compounds, generally recognized as stable by those skilled in the art, are within the scope of the present disclosure.

Unless otherwise stated, structures depicted herein are also meant to include compounds which differ only in the presence of one or more isotopically enriched atoms. For example, compounds having the present structures except for the replacement of a hydrogen by a deuterium or tritium, replacement of fluoride by ¹⁸F, or the replacement of a carbon by ¹³C- or ¹⁴C-enriched carbon are within the scope of the present disclosure.

The compounds provided herein may also contain unnatural proportions of atomic isotopes at one or more of the atoms that constitute such compounds. For example, the compounds may be radiolabeled with radioactive isotopes, such as for example tritium (H), iodine-125 (¹²⁵I), or carbon-14 (¹⁴C). All isotopic variations of the compounds provided herein, whether radioactive or not, are included within the present disclosure.

It should be noted that throughout the application that alternatives are written in Markush groups, for example, each amino acid position that contains more than one possible amino acid. It is specifically contemplated that each member of the Markush group should be considered separately, thereby comprising another embodiment, and the Markush group is not to be read as a single unit.

“Analog,” or “analogue” is used in accordance with its plain ordinary meaning within Chemistry and Biology and refers to a chemical compound that is structurally similar to another compound (i.e., a so-called “reference” compound) but differs in composition, e.g., in the replacement of one atom by an atom of a different element, or in the presence of a particular functional group, or the replacement of one functional group by another functional group, or the absolute stereochemistry of one or more chiral centers of the reference compound. Accordingly, an analog is a compound that is similar or comparable in function and appearance but not in structure or origin to a reference compound.

The terms “a” or “an,” as used in herein means one or more. In addition, the phrase “substituted with a[n],” as used herein, means the specified group may be substituted with one or more of any or all of the named substituents. For example, where a group, such as an alkyl or heteroaryl group, is “substituted with an unsubstituted C₁-C₂₀ alkyl, or unsubstituted 2 to 20 membered heteroalkyl,” the group may contain one or more unsubstituted C₁-C₂₀ alkyls, and/or one or more unsubstituted 2 to 20 membered heteroalkyls.

Where a moiety is substituted with an R substituent, the group may be referred to as “R-substituted.” Where a moiety is R-substituted, the moiety is substituted with at least one R substituent and each R substituent is optionally different. Where a particular R group is present in the description of a chemical genus (such as Formula (I)), a Roman decimal symbol may be used to distinguish each appearance of that particular R group. For example, where multiple R¹³ substituents are present, each R¹³ substituent may be distinguished as R^{13.1}, R^{13.2}, R^{13.3}, R^{13.4}, etc., wherein each of R^{13.1}, R^{13.2}, R^{13.3}, R^{13.4}, etc. is defined within the scope of the definition of R¹³ and optionally differently. The terms “a” or “an,” as used in herein means one or more. In addition, the phrase “substituted with a[n],” as used herein, means the specified group may be substituted with one or more of any or all of the named substituents. For example, where a group, such as an alkyl or heteroaryl group, is “substituted with an unsubstituted C₁-C₂₀ alkyl, or unsubstituted 2 to 20 membered heteroalkyl,” the group may contain one or more unsubstituted C₁-C₂₀ alkyls, and/or one or more unsubstituted 2 to 20 membered heteroalkyls.

Description of compounds of provided herein is limited by principles of chemical bonding known to those skilled in the art. Accordingly, where a group may be substituted by one or more of a number of substituents, such substitutions are selected so as to comply with principles of chemical bonding and to give compounds which are not inherently unstable and/or would be known to one of ordinary skill in the art as likely to be unstable under ambient conditions, such as aqueous, neutral, and several known physiological conditions. For example, a heterocycloalkyl or heteroaryl is attached to the remainder of the molecule via a ring heteroatom in compliance with principles of chemical bonding known to those skilled in the art thereby avoiding inherently unstable compounds.

### Compounds

Embodiments of the present disclosure relate to compounds targeted to the human peripheral myelin protein 22 (PMP22) mRNA (NCBI Reference Sequence NM_000304.4, deposited with GenBank on Nov. 22, 2018; SEQ ID NO: 1170). The compounds include double-stranded nucleic acids and single-stranded nucleic acids that act through the RNA interference pathway to inhibit the expression of the PMP22 mRNA. In embodiments, a compound is a double-stranded nucleic acid comprising an antisense strand complementary to the PMP22 mRNA and a sense strand complementary to the antisense strand. In embodiments, the antisense strand and sense strand of a compound are two separate strands and are not covalently linked and form a small interfering RNA (siRNA). In embodiments, the antisense strand and sense strand of a compound are covalently linked by a nucleotide linker to form a short hairpin RNA (shRNA). In embodiments, the compound is a single-stranded nucleic acid comprising an antisense strand complementary to the PMP22 mRNA (ssRNAi).

Provided herein are compounds comprising an antisense strand and a sense strand hybridized to form a double-stranded nucleic acid, wherein each of the antisense strand and sense strands is 15 to 25 nucleotides in length, the nucleotide sequence of the antisense strand is at least 90% complementary to the human peripheral myelin protein 22 mRNA (SEQ ID NO: 1170), and the nucleotide sequence of the sense strand has no more than two mismatches to the nucleotide sequence of the antisense strand in the double-stranded region.

Provided herein are compounds comprising an antisense strand and a sense strand hybridized to form a double-stranded nucleic acid, each of the antisense strand and sense strands is 15 to 25 nucleotides in length, the nucleotide sequence of the antisense strand comprises at least 15 contiguous nucleotides of a nucleotide sequence selected from any one of SEQ ID NOs 491, 492, 493, 494, 495, 497, 498, 503, 504, 506, 510, 511, 514, 515, 516, 518, 524, 526, 529, 531, 532, 533, 534, 535, 536, 538, 539, 540, 541, 542, 543, 545, 546, 547, 548, 550, 553, 554, 556, 558, 559, 560, 561, 563, 567, 569, 575, 576, 579, 580, 581, 582, 583, 585, 590, 591, 595, 597, 600, 605, 609, 610, 618, 622, 623, 628, 630, 631, 633, 635, 637, 639, 641, 642, 643, 644, 645, 1112, 1113, 1114, 1115, 1116, 1117, 1118, 1119, 1120, 1122, 1124, 1125, 1126, 1127, 1128, 1129, 1130, 1131, 1132, 1133, 1134, 1135, 1136, 1137, 1138, 1139, 1140, 1141, 1142, 1143, 1144, 1145, 1146, 1147, 1148, 1149, 1150, 1151, 1152, 1153, 1154, 1155, 1156, 1157, 1158, 1159, 1160, 1161, 1162, 1163, 1164, 1165, 1166, 1167, 1168, 1169, 1118, 1121, 1123, 1126, and 1144, and the nucleotide sequence of the sense strand has no more than two mismatches to the nucleotide sequence of the antisense strand.

Provided herein are compounds comprising a single-stranded nucleic acid comprising an antisense strand, wherein the antisense strand is 15 to 25 nucleotides in length and the nucleotide sequence of the antisense strand comprises at least 15 contiguous nucleotides of a nucleotide sequence selected from any one of SEQ ID NOs 491, 492, 493, 494, 495, 497, 498, 503, 504, 506, 510, 511, 514, 515, 516, 518, 524, 526, 529, 531, 532, 533, 534, 535, 536, 538, 539, 540, 541, 542, 543, 545, 546, 547, 548, 550, 553, 554, 556, 558, 559, 560, 561, 563, 567, 569, 575, 576, 579, 580, 581, 582, 583, 585, 590, 591, 595, 597, 600, 605, 609, 610, 618, 622, 623, 628, 630, 631, 633, 635, 637, 639, 641, 642, 643, 644, 645, 1112, 1113, 1114, 1115, 1116, 1117, 1118, 1119, 1120, 1122, 1124, 1125, 1126, 1127, 1128, 1129, 1130, 1131, 1132, 1133, 1134, 1135, 1136, 1137, 1138, 1139, 1140, 1141, 1142, 1143, 1144, 1145, 1146, 1147, 1148, 1149, 1150, 1151, 1152, 1153, 1154, 1155, 1156, 1157, 1158, 1159, 1160, 1161, 1162, 1163, 1164, 1165, 1166, 1167, 1168, 1169, 1118, 1121, 1123, 1126, and 1144.

In embodiments, the nucleotide sequence of the antisense strand comprises at least 16, at least 17, at least 18, at least 19, at least 20, at least 21, at least 22, or 23 contiguous nucleotides selected from any one of SEQ ID NOs 491, 492, 493, 494, 495, 497, 498, 503, 504, 506, 510, 511, 514, 515, 516, 518, 524, 526, 529, 531, 532, 533, 534, 535, 536, 538, 539, 540, 541, 542, 543, 545, 546, 547, 548, 550, 553, 554, 556, 558, 559, 560, 561, 563, 567, 569, 575, 576, 579, 580, 581, 582, 583, 585, 590, 591, 595, 597, 600, 605, 609, 610, 618, 622, 623, 628, 630, 631, 633, 635, 637, 639, 641, 642, 643, 644, 645, 1112, 1113, 1114, 1115, 1116, 1117, 1118, 1119, 1120, 1122, 1124, 1125, 1126, 1127, 1128, 1129, 1130, 1131, 1132, 1133, 1134, 1135, 1136, 1137, 1138, 1139, 1140, 1141, 1142, 1143, 1144, 1145, 1146, 1147, 1148, 1149, 1150, 1151, 1152, 1153, 1154, 1155, 1156, 1157, 1158, 1159, 1160, 1161, 1162, 1163, 1164, 1165, 1166, 1167, 1168, 1169, 1118, 1121, 1123, 1126, and 1144.

In embodiments, the nucleotide sequence of the antisense strand comprises 19 contiguous nucleotides of a nucleotide sequence selected from any one of SEQ ID NOs 491, 492, 493, 494, 495, 497, 498, 503, 504, 506, 510, 511, 514, 515, 516, 518, 524, 526, 529, 531, 532, 533, 534, 535, 536, 538, 539, 540, 541, 542, 543, 545, 546, 547, 548, 550, 553, 554, 556, 558, 559, 560, 561, 563, 567, 569, 575, 576, 579, 580, 581, 582, 583, 585, 590, 591, 595, 597, 600, 605, 609, 610, 618, 622, 623, 628, 630, 631, 633, 635, 637, 639, 641, 642, 643, 644, 645, 1112, 1113, 1114, 1115, 1116, 1117, 1118, 1119, 1120, 1122, 1124, 1125, 1126, 1127, 1128, 1129, 1130, 1131, 1132, 1133, 1134, 1135, 1136, 1137, 1138, 1139, 1140, 1141, 1142, 1143, 1144, 1145, 1146, 1147, 1148, 1149, 1150, 1151, 1152, 1153, 1154, 1155, 1156, 1157, 1158, 1159, 1160, 1161, 1162, 1163, 1164, 1165, 1166, 1167, 1168, 1169, 1118, 1121, 1123, 1126, and 1144.

Provided below are features of compounds, such as length, nucleotide sequence, and nucleotide modifications. It is understood that an embodiment of an antisense strand may apply to the antisense strand of a single-stranded nucleic acid or a double-stranded nucleic acid. Further, it is understood that an embodiment of a sense strand may apply to a sense strand of any double-stranded nucleic acid provided herein, including siRNAs and shRNAs.

In embodiments, an antisense strand is 15 to 25 nucleotides in length. In embodiments, an antisense strand is 17 to 23 nucleotides in length. In embodiments, an antisense strand is 19 to 21 nucleotides in length. In embodiments, an antisense strand is 21 to 23 nucleotides in length. In embodiments, an antisense strand is 15 nucleotides in length. In embodiments, an antisense strand is 16 nucleotides in length. In embodiments, an antisense strand is 17 nucleotides in length. In embodiments, an antisense strand is 18 nucleotides in length. In embodiments, an antisense strand is 19 nucleotides in length. In embodiments, an antisense strand is 20 nucleotides in length. In embodiments, an antisense strand is 21 nucleotides in length. In embodiments, an antisense strand is 22 nucleotides in length. In embodiments, an antisense strand is 23 nucleotides in length. In embodiments, an antisense strand is 24 nucleotides in length. In embodiments, an antisense strand is 25 nucleotides in length.

In embodiments, the nucleotide sequence of the antisense strand is at least 95% complementary to SEQ ID NO: 1170. In embodiments, the nucleotide sequence of the antisense strand is 100% complementary to SEQ ID NO: 1170. In embodiments, the nucleotide sequence of the antisense strand is 100% complementary to nucleotides 213 to 233 of SEQ ID NO: 1170.

In embodiments, a sense strand is 15 to 25 nucleotides in length. In embodiments, a sense strand is 17 to 23 nucleotides in length. In embodiments, a sense strand is 19 to 21 nucleotides in length. In embodiments, a sense strand is 21 to 23 nucleotides in length. In embodiments, a sense strand is 15 nucleotides in length. In embodiments, a sense strand is 16 nucleotides in length. In embodiments, a sense strand is 17 nucleotides in length. In embodiments, a sense strand is 18 nucleotides in length. In embodiments, a sense strand is 19 nucleotides in length. In embodiments, a sense strand is 20 nucleotides in length. In embodiments, a sense strand is 21 nucleotides in length. In embodiments, a sense strand is 22 nucleotides in length. In embodiments, a sense strand is 23 nucleotides in length. In embodiments, a sense strand is 24 nucleotides in length. In embodiments, a sense strand is 25 nucleotides in length.

In embodiments, length of the sense strand is identical to the length of the antisense strand. In embodiments, the length of the sense strand is greater than the length of the antisense strand. In embodiments, the length of the sense strand is less than the length of the antisense strand.

The double-stranded region of a double-stranded nucleic acid may be from 15 to 25 nucleobase pairs in length, depending on the lengths of the sense strand and the antisense strand. In embodiments, the double-stranded region is 17 to 23 nucleobase pairs in length. In embodiments, the double-stranded region is 19 to 21 nucleobase pairs in length. In embodiments, the double-stranded region is 21 to 23 nucleotides in length. In embodiments, the double-stranded region is 15 nucleobase pairs in length. In embodiments, the double-stranded region is 16 nucleobase pairs in length. In embodiments, the double-stranded region is 17 nucleobase pairs in length. In embodiments, the double-stranded region is 18 nucleobase pairs in length. In embodiments, the double-stranded region is 19 nucleobase pairs in length. In embodiments, the double-stranded region is 20 nucleobase pairs in length. In embodiments, the double-stranded region is 21 nucleobase pairs in length. In embodiments, the double-stranded region is 22 nucleobase pairs in length. In embodiments, the double-stranded region is 23 nucleobase pairs in length. In embodiments, the double-stranded region is 24 nucleobase pairs in length. In embodiments, the double-stranded region is 25 nucleobase pairs in length.

In embodiments, the nucleotide sequence of a sense strand has no more than one mismatch to the nucleotide sequence of an antisense strand of a double-stranded nucleic acid. In embodiments, the nucleotide sequence of a sense strand has no mismatches to the nucleotide sequence of an antisense strand of a double-stranded nucleic acid. Single-stranded nucleotide overhangs and nucleotide linkers are not considered for the purposes of determining the number of mismatches within the double-stranded region of a double-stranded nucleic acid provided herein. For example, a double-stranded nucleic acid comprising an antisense strand that is 23 nucleotides in length, and a sense strand that is 21 nucleotides in length have no mismatches over the double-stranded region, provided the nucleotide sequence of the sense strand is fully complementary over its length the nucleotide sequence of the antisense strand. Alternatively, a double-stranded nucleic acid comprising a sense strand that is 20 nucleotides in length, an antisense strand that is 22 nucleotides in length, and a nucleotide linker that is eight nucleotides in length, may have no mismatches over the double-stranded region provided the nucleotide sequence of the sense strand is fully complementary over its length to the nucleotide sequence of the antisense strand.

In embodiments, a double-stranded nucleic acid comprises an antisense strand of 19 nucleotides in length and a sense strand of 19 nucleotides in length. In embodiments, the antisense strand is 22 nucleotides in length and the sense strand is 20 nucleotides in length. In embodiments, the antisense strand is 23 nucleotides in length and the sense strand is 21 nucleotides in length. In embodiments, the antisense strand is 23 nucleotides in length including two deoxythymidines at the 3′ terminus, and the sense strand is 21 nucleotides in length including two deoxythymidines at the 3′ terminus.

In embodiments of compound comprising double-stranded nucleic acid where the antisense strand and sense strand are separate strands that are not covalently linked, the terminal nucleotides may form a nucleobase pair, in which case the end of the double-stranded nucleic acid is a blunt end. Alternatively, one or more unpaired nucleotides of an antisense strand and/or sense strand may extend beyond the terminus of the complementary strand, resulting in a nucleotide overhang of one or more terminal single-stranded nucleotides. In embodiments, at least one of the 5′ and 3′ terminus of a double-stranded nucleic acid is a blunt end. In embodiments, both the 5′ terminus and 3′ terminus of the double-stranded nucleic acid are blunt ends. In embodiments, at least one end of the double-stranded nucleic acid comprises a nucleotide overhang. In embodiments, each end of the double-stranded nucleic acid comprises a nucleotide overhang. In embodiments, one end of the double-stranded nucleic acid is a blunt end and the other end of the double-stranded nucleic acid comprises a nucleotide overhang. In embodiments, the antisense strand comprises a nucleotide overhang at its 3′ terminus. In embodiments, the sense strand comprises a nucleotide overhang at its 3′ terminus. In embodiments, each of the antisense strand and sense strand comprises a nucleotide overhang at its 3′ terminus. In embodiments, at least one of the antisense strand and sense strand comprises a nucleotide overhang at its 5′ terminus. In embodiments, each of the antisense strand and sense strand comprises a nucleotide overhang at each 5′ terminus.

In embodiments, a nucleotide overhang is from one to five single-stranded nucleotides. In embodiments, a nucleotide overhang is one single-stranded nucleotide. In embodiments, a nucleotide overhang is two single-stranded nucleotides. In embodiments, a nucleotide overhang is three single-stranded nucleotides. In embodiments, a nucleotide overhang is three single-stranded nucleotides. In embodiments, a nucleotide overhang is four single-stranded nucleotides. In embodiments, a nucleotide overhang is five single-stranded nucleotides. In embodiments, at least one of the single-stranded nucleotides of a nucleotide overhang is a modified nucleotide. In embodiments, each of the single-stranded nucleotides of a nucleotide overhang is a modified nucleotide. In embodiments, the modified nucleotide is a 2′-O-methyl nucleotide. In embodiments, the nucleotide overhang is two single-stranded nucleotides and each nucleotide is a 2′-O-methoxyethyl nucleotide.

In embodiments, at least one nucleotide of the nucleotide overhang at the 3′ terminus of an antisense strand is complementary to a corresponding nucleotide of SEQ ID NO: 1170. In embodiments, each nucleotide of the nucleotide overhang at the 3′ terminus of an antisense strand is complementary to a corresponding nucleotide of SEQ ID NO: 1170. In some embodiment, at least one nucleotide of the nucleotide overhang at the 3′ terminus of an antisense strand is not complementary to a corresponding nucleotide of SEQ ID NO: 1170. In embodiments, each nucleotide of the nucleotide overhang at the 3′ terminus of an antisense strand is not complementary to a corresponding nucleotide of SEQ ID NO: 1170.

In embodiments, at least one single-stranded nucleotide of a nucleotide overhang is a deoxythymidine nucleotide. In embodiments, a nucleotide overhang is two single-stranded nucleotides and each nucleotide is a deoxythymidine nucleotide. In embodiments, the nucleotide sequence of the antisense strand comprises a nucleotide overhang of two deoxythymidine nucleotides. In embodiments, the sense strand comprises a nucleotide overhang of two deoxythymidine nucleotides. In embodiments, the antisense strand and the sense strand comprise a nucleotide overhang of two deoxythymidine nucleotides.

Non-limiting examples of double-stranded nucleic acids comprising blunt ends or nucleotide overhangs are provided in Table 1 below.

In the first example, where the antisense strand is 21 nucleotides in length and the sense strand is 21 nucleotides in length, and the nucleotide sequence of the antisense strand is fully complementary to the nucleotide sequence of the sense strand over the double-stranded region, the length of the double-stranded region is 19 nucleobase pairs and each terminus of the double-stranded nucleic acid has a dTdT overhang.

In the second example, where the antisense strand is 21 nucleotides in length and the sense strand is 19 nucleotides in length, and the nucleotide sequence of the antisense strand is fully complementary to the nucleotide sequence of the sense strand over the double-stranded region, the length of the double-stranded region is 19 nucleobase pairs and the 3′ terminus of the antisense strand comprises a dTdT overhang.

In the third example, where the antisense strand is 19 nucleotides in length and the sense strand is 19 nucleotides in length, and the nucleotide sequence of the antisense strand is fully complementary to the nucleotide sequence of the sense strand over the double-stranded region, the length of the double-stranded region is 19 nucleobase pairs and each terminus is a blunt end.

In the fourth example, where the antisense strand is 23 nucleotides in length and the sense strand is 21 nucleotides in length, the length of the double-stranded region is 21 nucleobase pairs and 3′ terminus of the antisense strand comprises a two-nucleotide overhang.

TABLE 1
Examples of double-stranded nucleic acids

| | | | | | SEQ |
|---|---|---|---|---|---|
| | | Nb | Terminus | | ID |
| Strand | Length | Pairs | Type | Nucleotide sequence | NO: |
| Sense | 21 | 19 | Overhang/ | 5′-AAACCUAUUUAUAACACUUTT-3′ | 490 |
| Antisense | 21 | | Overhang | \|\|\|\|\|\|\|\|\|\|\|\|\|\|\|\|\|\|\| | 510 |
| | | | | 3′-TTUUUGGAUAAAUAUUGUGAA-5′ | |
| Sense | 19 | 19 | Overhang/ | 5′-AAACCUAUUUAUAACACUU-3′ | 976 |
| Antisense | 21 | | Blunt | \|\|\|\|\|\|\|\|\|\|\|\|\|\|\|\|\|\|\| | 510 |
| | | | | 3′-TTUUUGGAUAAAUAUUGUGAA-5′ | |
| Sense | 19 | 19 | Blunt/ | 5′-AAACCUAUUUAUAACACUU-3′ | 976 |
| Antisense | 19 | | Blunt | \|\|\|\|\|\|\|\|\|\|\|\|\|\|\|\|\|\|\| | nt |
| | | | | 3′-UUUGGAUAAAUAUUGUGAA-5′ | 1-19 |
| | | | | | of |
| | | | | | SEQ |
| | | | | | ID |
| | | | | | NO: |
| | | | | | 510 |
| Sense | 21 | 21 | Overhang/ | 5′-AAACGAAUGGCUGCAGUCUGU-3′ | 977 |
| Antisense | 23 | | Blunt | \|\|\|\|\|\|\|\|\|\|\|\|\|\|\|\|\|\|\|\|\| | 1125 |
| | | | | 3′-GGUUUGCUUACCGACGUCAGACA-5′ | |

In embodiments of a double-stranded nucleic acid comprising a nucleotide linker, the termini that are not connected by the nucleotide linker may form a blunt end or may form a nucleotide overhang of one or more single-stranded nucleotides. In embodiments, the non-linked end of the double-stranded nucleic acid is a blunt end. In embodiments, the non-linked end comprises a nucleotide overhang of one or more single-stranded nucleotides. In embodiments, the non-linked end of the guide strand comprises a nucleotide overhang. In embodiments, the non-linked end of the sense strand comprises a nucleotide overhang. In embodiments, the 3′ terminus of the guide strand comprises a nucleotide overhang. In embodiments, the 3′ terminus of the sense strand comprises a nucleotide overhang. In embodiments, the 5′ terminus of the sense strand comprises a nucleotide overhang. In embodiments, the 5′ terminus of the sense strand comprises a nucleotide overhang.

In embodiments of a double-stranded nucleic acid where the antisense and sense strand are covalently linked by a nucleotide linker, the nucleotide linker is four to 16 nucleotides in length. In embodiments, the nucleotide linker is four nucleotides in length. In embodiments, the nucleotide linker is four nucleotides in length. In embodiments, the nucleotide linker is five nucleotides in length. In embodiments, the nucleotide linker is six nucleotides in length. In embodiments, the nucleotide linker is seven nucleotides in length. In embodiments, the nucleotide linker is eight nucleotides in length. In embodiments, the nucleotide linker is nine nucleotides in length. In embodiments, the nucleotide linker is 10 nucleotides in length. In embodiments, the nucleotide linker is 11 nucleotides in length. In embodiments, the nucleotide linker is 12 nucleotides in length. In embodiments, the nucleotide linker is 13 nucleotides in length. In embodiments, the nucleotide linker is 14 nucleotides in length. In embodiments, the nucleotide linker is 15 nucleotides in length. In embodiments, the nucleotide linker is 16 nucleotides in length.

Although the sequence listing accompanying this filing identifies each nucleotide sequence as either “RNA” or “DNA” as required, in practice, those sequences may be modified with a combination of chemical modifications specified herein. One of skill in the art will readily appreciate that in the sequence listing, such designation as “RNA” or “DNA” to describe modified nucleotides is somewhat arbitrary. For example, a nucleic acid provided herein comprising a nucleotide comprising a 2′-O-methyl sugar moiety and a thymine base may be described as a DNA residue in the sequence listing, even though the nucleotide is modified and is not a naturally-occurring DNA nucleotide.

Accordingly, nucleic acid sequences provided in the sequence listing are intended to encompass nucleic acids containing any combination of natural or modified RNA and/or DNA, including, but not limited to such nucleic acids having modified nucleobases. By way of further example and without limitation, a nucleic acid having the nucleotide sequence “ATCGATCG” in the sequence listing encompasses any nucleic acid having such nucleotide sequence, whether modified or unmodified, including, but not limited to, such nucleic acids comprising RNA bases, such as those having sequence “AUCGAUCG” and those having some DNA bases and some RNA bases such as “AUCGATCG” and oligonucleotides having other modified bases, such as “ATmeCGAUCG,” wherein meC indicates a 5-methylcytosine.

### Modified Nucleotides

Double-stranded and single-stranded nucleic acids provided herein may comprise one or more modified nucleotides. A modified nucleotide may be selected over an unmodified form because of desirable properties such as, for example, enhanced cellular uptake, enhanced affinity for other oligonucleotides or nucleic acid targets, increased stability in the presence of nucleases, and/or reduced immune stimulation.

In embodiments, at least one nucleotide of the antisense strand is a modified nucleotide. In embodiments, at least one nucleotide of the sense strand is a modified nucleotide. In embodiments, each nucleotide of the antisense strand forming the double-stranded region is a modified nucleotide. In embodiments, each nucleotide of the sense strand forming the double-stranded region comprises is a modified nucleotide.

In embodiments, a modified nucleotide comprises one or more of a modified sugar moiety, a modified internucleotide linkage, and a 5′-terminal modified phosphate group. In embodiments, a modified nucleotide comprises a modified sugar moiety. In embodiments, a modified nucleotide comprises a modified internucleotide linkage. In embodiments, a modified nucleotide comprises a modified nucleobase. In embodiments, a modified nucleotide comprises a modified 5′-terminal phosphate group. In embodiments, a modified nucleotide comprises a modification at the 5′ carbon of the pentafuranosyl sugar. In embodiments, a modified nucleotide comprises a modification at the 3′ carbon of the pentafuranosyl sugar. In embodiments, a modified nucleotide comprises a modification at the 2′ carbon of the pentafuranosyl sugar. In embodiments, a modified nucleotide is at the 5′ terminus of an antisense strand or sense strand. In embodiments, a modified nucleotide is at the 3′ terminus of an antisense strand or sense strand. In embodiments, a modified nucleotide is at an internal nucleotide of an antisense strand or sense strand. In embodiments, a modified nucleotide comprises a ligand attached to the 2′, 3, or 5′ carbon of the pentafuranosyl sugar. In embodiments, a nucleotide comprises a ligand attached to a nucleobase.

A modified nucleotide may comprise a modified sugar moiety, a naturally occurring nucleobase, and a naturally occurring internucleotide linkage. A modified nucleotide may comprise a modified sugar moiety, a naturally occurring nucleobase, and a modified internucleotide linkage.

In embodiments, a modified sugar moiety is modified at the 2′ carbon of the pentafuranosyl sugar, relative to the naturally occurring 2′-OH of RNA or the 2′-H of DNA. In embodiments, a modification at the 2′ carbon of the pentafuranosyl sugar is selected from F, OCF₃, OCH₃ (also referred to as “2′—OMe” or “2′O-methyl), OCH₂CH₂OCH₃ (also referred to as “2′-O-methoxyethyl” or “2′-MOE”), 2′-O(CH₂)₂SCH₃, O—(CH₂)₂—O—N(CH₃)₂, —O(CH₂)₂O(CH₂)₂N(CH₃)₂, and O—CH₂—C(═O)—N(H)CH₃.

In embodiments, a modified sugar moiety is a 2′-fluoro sugar (also referred to as a 2′-F sugar). In embodiments, a modified sugar moiety is a 2′-O-methyl sugar (also referred to as a “2′-OMe sugar” or a “²′-OCH₃” sugar). In embodiments, a modified sugar moiety is a 2′-O-methoxyethyl sugar (also referred to as a 2′-OCH₂CH₂OCH₃ or a 2′-MOE sugar).

In embodiments, the modified nucleotide comprising a modified sugar moiety is selected from a 2′-fluoro nucleotide, a 2′-O-methyl nucleotide, a 2′-O-methoxyethyl nucleotide, and a bicyclic sugar nucleotide. In embodiments, a modified nucleotide is a 2′-fluoro nucleotide, where the 2′ carbon of the pentafuranosyl sugar has a fluoro substitution. In embodiments, a modified nucleotide is a 2′-O-methyl nucleotide, where the 2′ carbon of the pentafuranosyl sugar has a 2′-O methyl substitution. In embodiments, a modified nucleotide is a 2′-O-methoxyethyl nucleotide, where the 2′ carbon of the pentafuranosyl sugar has a 2′-O-methoxyethyl substitution. Other modified nucleotides may be similarly named.

In embodiments, a modified nucleotide comprises a modified sugar moiety, where the ribose has a covalent linkage between the 2′ and 4′ carbons. Such a modified sugar moiety may be referred to as a “bicyclic sugar,” and nucleotides comprising such sugar moieties may be referred to as “bicyclic nucleic acids.” In embodiments, the covalent linkage of a bicyclic sugar is a methyleneoxy linkage (4‘—C’H—O-2′), also known as “LNA.” In embodiments, the covalent linkage of a bicyclic sugar is an ethyleneoxy linkage (4′-(CH₂)₂—O-2′), also known as “ENA.” In embodiments, the covalent linkage of a bicyclic moiety is a methyl(methyleneoxy) linkage (4′-CH(CH₃)—O-2′), also known as “constrained ethyl” or “cEt.” In certain embodiments, the —CH(CH₃) bridge is constrained in the S orientation (“S-cEt”). In certain embodiments, the —H(CH₃)— bridge is constrained in the R orientation (“R-cft”). In embodiments, the covalent linkage of a bicyclic sugar is a (4′-CH(CH₂—OMe)-O-2′ linkage, also known as “c-MOE.” In embodiments, the bicyclic sugar is a D sugar in the alpha configuration. In certain such embodiments, the bicyclic sugar is a D sugar in the beta configuration. In certain such embodiments, the bicyclic sugar is an L sugar in the alpha configuration. In certain such embodiments, the bicyclic sugar is an L sugar in the beta configuration.

In embodiments, a modified sugar moiety is a 1,5-anhydrohexitol nucleic acid, also known as a “hexitol nucleic acid” or “HNA.”

In embodiments, the oxygen of the pentafuranosyl sugar is replace with a sulfur, to form a thio-sugar. In embodiments, a thio-sugar is modified at the 2′ carbon.

In embodiments, a modified internucleotide linkage is a phosphorothioate internucleotide linkage. In embodiments, a modified internucleotide linkage is a methylphosphonate internucleotide linkage.

In embodiments, the first two internucleotide linkages at the 5′ terminus of the sense strand and the last two internucleotide linkages at the 3′ terminus of the sense strand are phosphorothioate internucleotide linkages. In embodiments, the first two internucleotide linkages at the 5′ terminus of the antisense strand and the last two internucleotide linkages at the 3′ terminus of the antisense strand are phosphorothioate internucleotide linkages. In embodiments, the first two internucleotide linkages at the 5′ terminus of the sense strand and the last two internucleotide linkages at the 3′ terminus of the sense strand are phosphorothioate internucleotide linkages, and the first two internucleotide linkages at the 5′ terminus of the antisense strand and the last two internucleotide linkages at the 3′ terminus of the antisense strand are phosphorothioate internucleotide linkages.

In embodiments, a modified nucleobase is selected from 5-hydroxymethyl cytosine, 7-deazaguanine and 7-deazaadenine. In embodiments, a modified nucleobase is selected from 7-deaza-adenine, 7-deazaguanosine, 2-aminopyridine and 2-pyridone. In embodiments, a modified nucleobase is selected from 5-substituted pyrimidines, 6-azapyrimidines and N-2, N-6 and 0-6 substituted purines, including 2 aminopropyladenine, 5-propynyluracil and 5-propynylcytosine.

In embodiments, a modified nucleotide comprises a modification of the phosphate group at the 5′-carbon of the pentafuranosyl sugar. In embodiments, the modified phosphate group is 5′-(E)-vinylphosphonate (5′-VP).

In embodiments, a modified nucleotide is a phosphorodiamidite-linked morpholino nucleotide.

In embodiments, a modified nucleotide comprises an acyclic nucleoside derivative lacking the bond between the 2′ carbon and 3′ carbon of the sugar ring, also known as an “unlocked nucleic acid” or “UNA.”

In embodiments, the antisense strand is 21 nucleotides in length and the nucleotides of the antisense strand are modified such that, counting from the 5′ terminus of the antisense strand, nucleotides 1, 3, 5, 7, 9, 11, 13, 15, 17, and 19 are 2′-O-methyl nucleotides, nucleotides 2, 4, 6, 8, 10, 12, 14, 16, and 18 are 2′-fluoro nucleotides, and nucleotides 20 and 21 are beta-D-deoxynucleotides, the first two internucleotide linkages at the 5′ terminus and the last two internucleotide linkages at the 3′ terminus are phosphorothioate internucleotide linkages, and each other internucleotide linkage is a phosphodiester internucleotide linkage. Such a modification pattern may be represented by the following Pattern I:

5′-N_{M}^{S}N_{F}^{S}N_{M}N_{F}N_{M}N_{F}N_{M}N_{F}N_{M}N_{F}N_{M}N_{F}N_{M}N_{F}N_{M}N_{F}N_{M}N_{F}N_{M}^{S}N^{S}N-3′, wherein “N_{M}” is a 2′-O-methyl nucleotide, “N_{F}” is a 2′-fluoro nucleotide, “N” is a beta-D-deoxynucleotide, a superscript “S” is a phosphorothioate internucleotide linkage, and each other internucleotide linkage is a phosphodiester internucleotide linkage.

In embodiments, the sense strand is 21 nucleotides in length and the nucleotides of the sense strand are modified such that, counting from the from the 5′ terminus of the sense strand, nucleotides 1, 3, 5, 7, 9, 11, 13, 15, 17, and 19 are 2′-fluoro nucleotides, nucleotides 2, 4, 6, 8, 10, 12, 14, 16, and 18 are 2′-O-methyl nucleotides, and nucleotides 20 and 21 are beta-D-deoxynucleotides, the first two internucleotide linkages at the 5′ terminus and the last two internucleotide linkages at the 3′ terminus are phosphorothioate internucleotide linkages, and each other internucleotide linkage is a phosphodiester internucleotide linkage. Such a modification pattern may be represented by the following Pattern II:

5′-N_{F}^{S}N_{M}^{S}N_{F}N_{M}N_{F}N_{M}N_{F}N_{M}N_{F}N_{M}N_{F}N_{M}N_{F}N_{M}N_{F}N_{M}N_{F}N_{M}N_{F}^{S}N^{S}N-3′, wherein “N_{M}” is a 2′-O-methyl nucleotide, “N_{F}” is a 2′-fluoro nucleotide, “N” is a beta-D-deoxynucleotide, a superscript “S” is a phosphorothioate internucleotide linkage, and each other internucleotide linkage is a phosphodiester internucleotide linkage.

In embodiments, the antisense strand is 19 nucleotides in length and the nucleotides of the antisense strand are modified such that, counting from the 5′ terminus of the antisense strand, nucleotides 1, 3, 5, 7, 9, 11, 13, 15, 17, and 19 are 2′-O-methyl nucleotides and nucleotides 2, 4, 6, 8, 10, 12, 14, 16, and 18 are 2′-fluoro nucleotides, the first two internucleotide linkages at the 5′ terminus and the last two internucleotide linkages at the 3′ terminus are phosphorothioate internucleotide linkages, and each other internucleotide linkage is a phosphodiester internucleotide linkage. Such a modification pattern may be represented by the following Pattern III:

5′-N_{M}^{S}N_{F}^{S}N_{M}N_{F}N_{M}N_{F}N_{M}N_{F}N_{M}N_{F}N_{M}N_{F}N_{M}N_{F}N_{M}N_{F}N_{M}^{S}N_{F}^{S}N_{M}-3′, wherein “N_{M}” is a 2′-O-methyl nucleotide, “N_{F}” is a 2′-fluoro nucleotide, “N” is a beta-D-deoxynucleotide, a superscript “S” is a phosphorothioate internucleotide linkage, and each other internucleotide linkages is a phosphodiester internucleotide linkage.

In embodiments, the sense strand is 19 nucleotides in length and the nucleotides of the sense strand are modified such that, counting from the 5′ terminus of the sense strand, nucleotides 1, 3, 5, 7, 9, 11, 13, 15, 17, and 19 are 2′-fluoro nucleotides and nucleotides 2, 4, 6, 8, 10, 12, 14, 16, and 18 are 2′-O-methyl nucleotides, the first two internucleotide linkages at the 5′ terminus and the last two internucleotide linkages at the 3′ terminus are phosphorothioate internucleotide linkages, and each other internucleotide is a phosphodiester internucleotide linkage. Such a modification pattern may be represented by the following Pattern IV:

5′-N_{F}^{S}N_{M}^{S}N_{F}N_{M}N_{F}N_{M}N_{F}N_{M}N_{F}N_{M}N_{F}N_{M}N_{F}N_{M}N_{F}N_{M}N_{F}^{S}N_{M}^{S}N_{F}-3′, wherein “N_{M}” is a 2′-O-methyl nucleotide, “N_{F}” is a 2′-fluoro nucleotide, “N” is a beta-D-deoxynucleotide, a superscript “S” is a phosphorothioate internucleotide linkage, and each other internucleotide linkage is a phosphorodiester internucleotide linkage.

In embodiments, the antisense strand is 23 nucleotides in length and the nucleotides of the antisense strand are modified such that, counting from the 5′ terminus of the antisense strand, nucleotides 1, 3, 5, 7, 9, 11, 13, 15, 17, 19, 21, 22, and 23 are 2′-O-methyl nucleotides and nucleotides 2, 4, 6, 8, 10, 12, 14, 16, 18, and 20 are 2′-fluoro nucleotides the first two internucleotide linkages at the 5′ terminus and the last two internucleotide linkages at the 3′ terminus are phosphorothioate internucleotide linkages, and each other internucleotide linkage is a phosphodiester internucleotide linkage. Such a modification pattern may be represented by the following Pattern V:

5′-N_{M}^{S}N_{F}^{S}N_{M}N_{F}N_{M}N_{F}N_{M}N_{F}N_{M}N_{F}N_{M}N_{F}N_{M}N_{F}N_{M}N_{F}N_{M}N_{F}N_{M}N_{F}N_{M}^{S}N_{M}^{S}N_{M}-3′, wherein “N_{M}” is a 2′-O-methyl nucleotide, “N_{F}” is a 2′-fluoro nucleotide, a superscript “S” is a phosphorothioate internucleotide linkage, and each other internucleotide linkage is a phosphodiester internucleotide linkage.

In embodiments, wherein the sense strand is 21 nucleotides in length and the nucleotides of the sense strand are modified such that, counting from the 5′ terminus of the sense strand, nucleotides 1, 3, 5, 7, 9, 11, 13, 15, 17, 19, and 21 are 2′-fluoro nucleotides, nucleotides 2, 4, 6, 8, 10, 12, 14, 16, 18, and 20 are 2′-O-methyl nucleotides, the first two internucleotide linkages at the 5′ terminus and the last two internucleotide linkages at the 3′ terminus are phosphorothioate internucleotide linkages, and each other internucleotide linkage is a phosphodiester internucleotide linkage. Such a modification pattern may be represented by the Pattern VI:

5′-N_{F}^{S}N_{M}^{S}N_{F}N_{M}N_{F}N_{M}N_{F}N_{M}N_{F}N_{M}N_{F}N_{M}N_{F}N_{M}N_{F}N_{M}N_{F}N_{M}N_{F}^{S}N_{M}^{S}N_{F}-3′, wherein “N_{M}” is a 2′-O-methyl nucleotide, “N_{F}” is a 2′-fluoro nucleotide, a superscript “S” is a phosphorothioate internucleotide linkage, and each other internucleotide linkage is a phosphodiester internucleotide linkage.

In embodiments, the antisense strand is 23 nucleotides in length and the nucleotides of the antisense strand are modified such that, counting from the 5′ terminus of the antisense strand, nucleotides 1, 3, 5, 7, 9, 11, 12, 13, 15, 17, 19, 21, 22, and 23 are 2′-O-methyl nucleotides and nucleotides 2, 4, 6, 8, 10, 14, 16, 18, and 20 are 2′-fluoro nucleotides, the first two internucleotide linkages at the 5′ terminus and the last two internucleotide linkages at the 3′ terminus are phosphorothioate internucleotide linkages, and each other internucleotide linkage is a phosphodiester internucleotide linkage. Such a modification pattern may be represented by the Pattern VII:

5′-N_{M}^{S}N_{F}^{S}N_{M}N_{F}N_{M}N_{F}N_{M}N_{F}N_{M}N_{F}NMN_{M}N_{M}N_{F}N_{M}N_{F}N_{M}N_{F}N_{M}N_{F}N_{M}^{S}N_{M}^{S}N_{M}-3′, wherein “N_{M}” is a 2′-O-methyl nucleotide, “N_{F}” is a 2′-fluoro nucleotide, a superscript “S” is a phosphorothioate internucleotide linkage, and each other internucleotide linkage is a phosphodiester internucleotide linkage.

In embodiments, the sense strand is 21 nucleotides in length and the nucleotides of the sense strand are modified such that, counting from the 5′ terminus of the sense strand, nucleotides 1, 3, 5, 7, 9, 10, 11, 13, 15, 17, 19, and 21 are 2′-fluoronucleotides, nucleotides 2, 4, 6, 8, 12, 14, 16, 18, and 20 are 2′-O-methyl nucleotides, the first two internucleotide linkages at the 5′ terminus and the last two internucleotide linkages at the 3′ terminus are phosphorothioate internucleotide linkages, and each other internucleotide linkage is a phosphodiester internucleotide linkage. Such a modification pattern may be represented by the Pattern VIII:

5′-N_{F}^{S}N_{M}^{S}N_{F}N_{M}N_{F}N_{M}N_{F}N_{M}N_{F}N_{F}N_{F}N_{M}N_{F}N_{M}N_{F}N_{M}N_{F}N_{M}N_{F}^{S}N_{M}^{S}N_{F}-3′, wherein “N_{M}” is a 2′-O-methyl nucleotide, “N_{F}” is a 2′-fluoro nucleotide, a superscript “S” is a phosphorothioate internucleotide linkage, and each other internucleotide linkage is a phosphodiester internucleotide linkage.

In embodiments, the antisense strand is 23 nucleotides in length and the nucleotides of the antisense strand are modified such that, counting from the 5′ terminus of the antisense strand, nucleotides 1, 3, 5, 7, 9, 10, 11, 13, 15, 17, 19, 21, 22, and 23 are 2′-O-methyl nucleotides and nucleotides 2, 4, 6, 8, 12, 14, 16, 18, and 20 are 2′-fluoro nucleotides, the first two internucleotide linkages at the 5′ terminus and the last two internucleotide linkages at the 3′ terminus are phosphorothioate internucleotide linkages, and each other internucleotide linkage is a phosphodiester internucleotide linkage. Such a modification pattern may be represented by the Pattern IX:

5′-N_{M}^{S}N_{F}^{S}N_{M}N_{F}N_{M}N_{F}N_{M}N_{F}N_{M}N_{M}N_{M}N_{F}N_{M}N_{F}N_{M}N_{F}N_{M}N_{F}N_{M}N_{F}N_{M}^{S}N_{M}^{S}N_{M}-3′, wherein “N_{M}” is a 2′-O-methyl nucleotide, “N_{F}” is a 2′-fluoro nucleotide, a superscript “S” is a phosphorothioate internucleotide linkage, and each other internucleotide linkage is a phosphodiester internucleotide linkage.

In embodiments, the sense strand is 21 nucleotides in length and the nucleotides of the sense strand are modified such that, counting from the 5′ terminus of the sense strand, nucleotides 1, 3, 5, 7, 9, 11, 12, 13, 15, 17, 19, and 21 are 2′-fluoronucleotides, nucleotides 2, 4, 6, 8, 10, 14, 16, 18, and 20 are 2′-O-methyl nucleotides, the first two internucleotide linkages at the 5′ terminus and the last two internucleotide linkages at the 3′ terminus are phosphorothioate internucleotide linkages, and each other internucleotide linkage is a phosphodiester internucleotide linkage. Such a modification pattern may be represented by the Pattern X:

5′-N_{F}^{S}N_{M}^{S}N_{F}N_{M}N_{F}N_{M}N_{F}N_{M}N_{F}N_{M}N_{F}N_{F}N_{F}N_{M}N_{F}N_{M}N_{F}N_{M}N_{F}^{S}N_{M}^{S}N_{F}-3′, wherein “N_{M}” is a 2′-O-methyl nucleotide, “N_{F}” is a 2′-fluoro nucleotide, a superscript “S” is a phosphorothioate internucleotide linkage, and each other internucleotide linkage is a phosphodiester internucleotide linkage.

In embodiments, the sense strand is 23 nucleotides in length and the nucleotides of the sense strand are modified such that, counting from the 5′ terminus of the sense strand, nucleotides 1, 3, 5, 7, 9, 11, 13, 15, 17, 19, and 21 are 2′-fluoronucleotides, nucleotides 2, 4, 6, 8, 10, 12, 14, 16, 18, and 20 are 2′-O-methyl nucleotides, nucleotides 22 and 23 are beta-D-deoxynucleotides, the first two internucleotide linkages at the 5′ terminus and the last two internucleotide linkages at the 3′ terminus are phosphorothioate internucleotide linkages, and each other internucleotide linkage is a phosphodiester internucleotide linkage. Such a modification pattern may be represented by the Pattern XI:

5′-N_{F}^{S}N_{M}^{S}N_{F}N_{M}N_{F}N_{M}N_{F}N_{M}N_{F}N_{M}N_{F}N_{M}N_{F}N_{M}N_{F}N_{M}N_{F}N_{M}N_{F}N_{M}N_{F}^{S}N^{S}N-3′, wherein “N_{M}” is a 2′-O-methyl nucleotide, “N_{F}” is a 2′-fluoro nucleotide, “N” is a beta-D-deoxynucleotide, a superscript “S” is a phosphorothioate internucleotide linkage, and each other internucleotide linkage is a phosphodiester internucleotide linkage.

In embodiments, the sense strand is 23 nucleotides in length and the nucleotides of the sense strand are modified such that, counting from the 5′ terminus of the sense strand, nucleotides 1, 3, 5, 7, 9, 10, 11, 13, 15, 17, 19, and 21 are 2′-fluoronucleotides, nucleotides 2, 4, 6, 8, 12, 14, 16, 18, and 20 are 2′-O-methyl nucleotides, nucleotides 22 and 23 are beta-D-deoxynucleotides, the first two internucleotide linkages at the 5′ terminus and the last two internucleotide linkages at the 3′ terminus are phosphorothioate internucleotide linkages, and each other internucleotide linkage is a phosphodiester internucleotide linkage. Such a modification pattern may be represented by the Pattern XII:

5′-N_{F}^{S}N_{M}^{S}N_{F}N_{M}N_{F}N_{M}N_{F}N_{M}N_{F}N_{F}N_{F}N_{M}N_{F}N_{M}N_{F}N_{M}N_{F}N_{M}N_{F}N_{M}N_{F}^{S}N^{S}N-3′, wherein “N_{M}” is a 2′-O-methyl nucleotide, “N_{F}” is a 2′-fluoro nucleotide, “N” is a beta-D-deoxynucleotide, a superscript “S” is a phosphorothioate internucleotide linkage, and each other internucleotide linkage is a phosphodiester internucleotide linkage.

In embodiments, the sense strand is 23 nucleotides in length and the nucleotides of the sense strand are modified such that, counting from the 5′ terminus of the sense strand, nucleotides 1, 3, 5, 7, 9, 11, 12, 13, 15, 17, 19, and 21 are 2′-fluoronucleotides, nucleotides 2, 4, 6, 8, 10, 14, 16, 18, and 20 are 2′-O-methyl nucleotides, nucleotides 22 and 23 are beta-D-deoxynucleotides, the first two internucleotide linkages at the 5′ terminus and the last two internucleotide linkages at the 3′ terminus are phosphorothioate internucleotide linkages, and each other internucleotide linkage is a phosphodiester internucleotide linkage. Such a modification pattern may be represented by the Pattern XIII:

5′-N_{F}^{S}N_{M}^{S}N_{F}N_{M}N_{F}N_{M}N_{F}N_{M}N_{F}N_{M}N_{F}N_{F}N_{F}N_{M}N_{F}N_{M}N_{F}N_{M}N_{F}N_{M}N_{F}^{S}N^{S}N-3′, wherein “N_{M}” is a 2′-O-methyl nucleotide, “N_{F}” is a 2′-fluoro nucleotide, “N” is a beta-D-deoxynucleotide, a superscript “S” is a phosphorothioate internucleotide linkage, and each other internucleotide linkage is a phosphodiester internucleotide linkage.

In embodiments, the sense strand is 21 nucleotides in length and the nucleotides of the sense strand are modified such that, counting from the 5′ terminus of the sense strand, nucleotides 1, 2, 3, 4, 6, 8, 12, 14, 16, 18, 19, 20, and 21 are 2′-methyl nucleotides, nucleotides 5, 7, 9, 10, 11, 13, 15, and 17 are 2′-fluoro nucleotides, the first two internucleotide linkages at the 5′ terminus and the last two internucleotide linkages at the 3′ terminus are phosphorothioate internucleotide linkages, and each other internucleotide linkage is a phosphodiester internucleotide linkage. Such a modification pattern may be represented by the Pattern XIV:

5′-N_{M}^{S}N_{M}^{S}N_{M}N_{M}N_{F}N_{M}N_{F}N_{M}N_{F}N_{M}N_{F}N_{M}N_{F}N_{M}N_{F}N_{M}N_{F}N_{M}N_{M}^{S}N_{M}^{S}N_{M}-3′, wherein “N_{M}” is a 2′-O-methyl nucleotide, “N_{F}” is a 2′-fluoro nucleotide, a superscript “S” is a phosphorothioate internucleotide linkage, and each other internucleotide linkage is a phosphodiester internucleotide linkage.

In embodiments, the sense strand is 21 nucleotides in length and the nucleotides of the sense strand are modified such that, counting from the 5′ terminus of the sense strand, nucleotides 1, 2, 3, 4, 6, 8, 12, 14, 16, 18, 19, 20, and 21 are 2′-O-methyl nucleotides, nucleotides 5, 7, 9, 10, 11, 13, 15, and 17 are 2′-fluoro nucleotides, the first two internucleotide linkages at the 5′ terminus and the last two internucleotide linkages at the 3′ terminus are phosphorothioate internucleotide linkages, and each other internucleotide linkage is a phosphodiester internucleotide linkage. Such a modification pattern may be represented by the Pattern XV:

5′-N_{M}^{S}N_{M}^{S}N_{M}N_{M}N_{F}N_{M}N_{F}N_{M}N_{F}N_{M}N_{F}N_{M}N_{F}N_{M}N_{F}N_{M}N_{F}N_{M}N_{M}^{S}N_{M}^{S}N_{M}-3′, wherein “N_{M}” is a 2′-O-methyl nucleotide, “N_{F}” is a 2′-fluoro nucleotide, a superscript “S” is a phosphorothioate internucleotide linkage, and each other internucleotide linkage is a phosphodiester internucleotide linkage.

In embodiments, the antisense strand is 23 nucleotides in length and the nucleotides of the antisense strand are modified such that, counting from the 5′ terminus of the antisense strand, nucleotides 1, 3, 5, 7, 8, 9, 11, 12, 13, 15, 17, 19, 21, 22, and 23 are 2′-O-methyl nucleotides, nucleotides 2, 4, 6, 10, 14, 16, 18, and 20 are 2′-fluoro nucleotides, the first two internucleotide linkages at the 5′ terminus and the last two internucleotide linkages at the 3′ terminus are phosphorothioate internucleotide linkages, and each other internucleotide linkage is a phosphodiester internucleotide linkage. Such a modification pattern may be represented by the Pattern XVI:

5′-N_{M}^{S}N_{F}^{S}N_{M}N_{F}N_{M}N_{F}N_{M}N_{F}N_{M}N_{M}N_{M}N_{F}N_{M}N_{F}N_{M}N_{F}N_{M}N_{F}N_{M}N_{F}N_{M}^{S}N_{M}^{S}N_{M}-3′, wherein “N_{M}” is a 2′-O-methyl nucleotide, “N_{F}” is a 2′-fluoro nucleotide, “N” is a beta-D-deoxynucleotide, a superscript “S” is a phosphorothioate internucleotide linkage, and each other internucleotide linkage is a phosphodiester internucleotide linkage.

In embodiments, the antisense strand is 23 nucleotides in length and the nucleotides of the antisense strand are modified such that, counting from the 5′ terminus of the antisense strand, nucleotides 1, 3, 4, 5, 7, 8, 9, 10, 11, 12, 13, 15, 17, 18, 19, 20, 21, 22, and 23 are 2′-O-methyl nucleotides, nucleotides 2, 6, 14, and 16 are 2′-fluoro nucleotides, the first two internucleotide linkages at the 5′ terminus and the last two internucleotide linkages at the 3′ terminus are phosphorothioate internucleotide linkages, and each other internucleotide linkage is a phosphodiester internucleotide linkage. Such a modification pattern may be represented by the Pattern XVII:

5′-N_{M}^{S}N_{F}^{S}N_{M}N_{F}N_{M}N_{F}N_{M}N_{F}N_{M}N_{M}N_{M}N_{F}N_{M}N_{F}N_{M}N_{F}N_{M}N_{F}N_{M}N_{F}N_{M}^{S}N_{M}^{S}N_{M}-3′, wherein “N_{M}” is a 2′-O-methyl nucleotide, “N_{F}” is a 2′-fluoro nucleotide, “N” is a beta-D-deoxynucleotide, a superscript “S” is a phosphorothioate internucleotide linkage, and each other internucleotide linkage is a phosphodiester internucleotide linkage.

In embodiments, the sense strand is 21 nucleotides in length and the nucleotides of the sense strand are modified such that, counting from the 5′ terminus of the sense strand, nucleotides 1, 2, 3, 4, 5, 6, 8, 12, 13, 14, 15, 16, 17, 18, 19, 20, and 21 are 2′-O-methyl nucleotides, nucleotides 7, 9, 10, and 11 are 2′-fluoro nucleotides, the first two internucleotide linkages at the 5′ terminus and the last two internucleotide linkages at the 3′ terminus are phosphorothioate internucleotide linkages, and each other internucleotide linkage is a phosphodiester internucleotide linkage. Such a modification pattern may be represented by the Pattern XVIII:

5′-N_{M}^{S}N_{M}^{S}N_{M}N_{M}N_{F}N_{M}N_{F}N_{M}N_{F}N_{M}N_{F}N_{M}N_{F}N_{M}N_{F}N_{M}N_{F}N_{M}N_{M}^{S}N_{M}^{S}N_{M}-3′, wherein “N_{M}” is a 2′-O-methyl nucleotide, “N_{F}” is a 2′-fluoro nucleotide, a superscript “S” is a phosphorothioate internucleotide linkage, and each other internucleotide linkage is a phosphodiester internucleotide linkage.

In embodiments, the sense strand is 21 nucleotides in length and the nucleotides of the sense strand are modified such that, counting from the 5′ terminus of the sense strand, nucleotides 1, 2, 3, 4, 5, 6, 8, 12, 13, 14, 15, 16, 17, 18, 19, 20, and 21 are 2′-O-methyl nucleotides, nucleotides 7, 9, 10, and 11 are 2′-fluoro nucleotides, the first two internucleotide linkages at the 5′ terminus are phosphorothioate internucleotide linkages, and each other internucleotide linkage is a phosphodiester internucleotide linkage. Such a modification pattern may be represented by the Pattern XIX:

5′-N_{M}^{S}N_{M}^{S}N_{M}N_{M}N_{F}N_{M}N_{F}N_{M}N_{F}N_{M}N_{F}N_{M}N_{F}N_{M}N_{F}N_{M}N_{F}N_{M}N_{M}^{S}N_{M}^{S}N_{M}-3′, wherein “N_{M}” is a 2′-O-methyl nucleotide, “N_{F}” is a 2′-fluoro nucleotide, a superscript “S” is a phosphorothioate internucleotide linkage, and each other internucleotide linkage is a phosphodiester internucleotide linkage.

In embodiments, the sense strand is 21 nucleotides in length and the nucleotides of the sense strand are modified such that, counting from the 5′ terminus of the sense strand, nucleotides 1 and 2 are 2′-O-methoxyethyl nucleotides, nucleotides 3, 4, 6, 8, 12, 14, 16, 18, 19, 20, and 21 are 2′-O-methyl nucleotides, nucleotides 5, 7, 9, 10, 11, 13, 15, and 17 are 2′-fluoro nucleotides, the first two internucleotide linkages at the 5′ terminus and the last two internucleotide linkages at the 3′ terminus are phosphorothioate internucleotide linkages, and each other internucleotide linkage is a phosphodiester internucleotide linkage. Such a modification pattern may be represented by the Pattern XX:

5′-N_{E}^{S}N_{E}^{S}N_{M}N_{M}N_{F}N_{M}N_{F}N_{M}N_{F}N_{M}N_{F}N_{M}N_{F}N_{M}N_{F}N_{M}N_{F}N_{M}N_{M}^{S}N_{M}^{S}N_{M}-3′, wherein “NE” is a 2′-O-methoxyethyl nucleotide, “N_{M}” is a 2′-O-methyl nucleotide, “N_{F}” is a 2′-fluoro nucleotide, a superscript “S” is a phosphorothioate internucleotide linkage, and each other internucleotide linkage is a phosphodiester internucleotide linkage.

In embodiments, the sense strand is 21 nucleotides in length and the nucleotides of the sense strand are modified such that, counting from the 5′ terminus of the sense strand, nucleotides 2 and 3 are 2′-O-methoxyethyl nucleotides, nucleotides 1, 4, 6, 8, 12, 14, 16, 18, 19, 20, and 21 are 2′-O-methyl nucleotides, nucleotides 5, 7, 9, 10, 11, 13, 15, and 17 are 2′-fluoro nucleotides, the first two internucleotide linkages at the 5′ terminus and the last two internucleotide linkages at the 3′ terminus are phosphorothioate internucleotide linkages, and each other internucleotide linkage is a phosphodiester internucleotide linkage. Such a modification pattern may be represented by the Pattern XXI:

5′-N_{E}^{S}N_{E}^{S}N_{M}N_{M}N_{F}N_{M}N_{F}N_{M}N_{F}N_{M}N_{F}N_{M}N_{F}N_{M}N_{F}N_{M}N_{F}N_{M}N_{M}^{S}N_{M}^{S}N_{M}-3′, wherein “NE” is a 2′-O-methoxyethyl nucleotide, “N_{M}” is a 2′-O-methyl nucleotide, “N_{F}” is a 2′-fluoro nucleotide, a superscript “S” is a phosphorothioate internucleotide linkage, and each other internucleotide linkage is a phosphodiester internucleotide linkage.

In embodiments, the sense strand is 21 nucleotides in length and the nucleotides of the sense strand are modified such that, counting from the 5′ terminus of the sense strand, nucleotides 2, 3, 19 and 20 are 2′-O-methoxyethyl nucleotides, nucleotides 1, 4, 6, 8, 12, 14, 16, 18, and 21 are 2′-O-methyl nucleotides, nucleotides 5, 7, 9, 10, 11, 13, 15, and 17 are 2′-fluoro nucleotides, the first two internucleotide linkages at the 5′ terminus and the last two internucleotide linkages at the 3′ terminus are phosphorothioate internucleotide linkages, and each other internucleotide linkage is a phosphodiester internucleotide linkage. Such a modification pattern may be represented by the Pattern XXII:

5′-N_{E}^{S}N_{E}^{S}N_{M}N_{M}N_{F}N_{M}N_{F}N_{M}N_{F}N_{M}N_{F}N_{M}N_{F}N_{M}N_{F}N_{M}N_{F}N_{M}N_{M}^{S}N_{M}^{S}N_{M}-3′, wherein “NE” is a 2′-O-methoxyethyl nucleotide, “N_{M}” is a 2′-O-methyl nucleotide, “N_{F}” is a 2′-fluoro nucleotide, a superscript “S” is a phosphorothioate internucleotide linkage, and each other internucleotide linkage is a phosphodiester internucleotide linkage.

In embodiments, the sense strand is 21 nucleotides in length and the nucleotides of the sense strand are modified such that, counting from the 5′ terminus of the sense strand, nucleotides 1, 2, 3, and 4 are 2′-O-methoxyethyl nucleotides, nucleotides 6, 8, 12, 14, 16, 18, 19, 20 and 21 are 2′-O-methyl nucleotides, nucleotides 5, 7, 9, 10, 11, 13, 15, and 17 are 2′-fluoro nucleotides, the first two internucleotide linkages at the 5′ terminus and the last two internucleotide linkages at the 3′ terminus are phosphorothioate internucleotide linkages, and each other internucleotide linkage is a phosphodiester internucleotide linkage. Such a modification pattern may be represented by the Pattern XXIII:

5′-N_{E}^{S}N_{E}^{S}N_{M}N_{M}N_{F}N_{M}N_{F}N_{M}N_{F}N_{M}N_{F}N_{M}N_{F}N_{M}N_{F}N_{M}N_{F}N_{M}N_{M}^{S}N_{M}^{S}N_{M}-3′, wherein “NE” is a 2′-O-methoxyethyl nucleotide, “N_{M}” is a 2′-O-methyl nucleotide, “N_{F}” is a 2′-fluoro nucleotide, a superscript “S” is a phosphorothioate internucleotide linkage, and each other internucleotide linkage is a phosphodiester internucleotide linkage.

In embodiments, an antisense strand has the modification pattern of Pattern I and a 5′-VP at the 5′-terminal nucleotide. In embodiments, an antisense strand has the modification pattern of Pattern III and a 5′-VP at the 5′-terminal nucleotide. In embodiments, an antisense strand has the modification pattern of Pattern V and a 5′-VP at the 5′-terminal nucleotide. In embodiments, an antisense strand has the modification pattern of Pattern VII and a 5′-VP at the 5′ terminal nucleotide. In embodiments, an antisense strand has the modification pattern of Pattern IX and a 5′-VP at the 5′ terminal nucleotide. In embodiments, an antisense strand has the modification pattern of Pattern XVI and a 5′-VP at the 5′ terminal nucleotide. In embodiments, an antisense strand has the modification pattern of Pattern XVII and a 5′-VP at the 5′ terminal nucleotide.

In embodiments, a compound comprises an antisense strand and a sense strand hybridized to form a double-stranded region, wherein the antisense strand and sense strand are not covalently linked (i.e. the antisense strand and sense strand form an siRNA), wherein the antisense strand is 21 nucleotides in length and the nucleotides of the antisense strand are modified such that, counting from the 5′ terminus of the antisense strand, nucleotides 1, 3, 5, 7, 9, 11, 13, 15, 17, and 19 are 2′-O-methyl nucleotides, nucleotides 2, 4, 6, 8, 10, 12, 14, 16, and 18 are 2′-fluoro nucleotides, and nucleotides 20 and 21 are beta-D-deoxynucleotides, the first two internucleotide linkages at the 5′ terminus and the last two internucleotide linkages at the 3′ terminus are phosphorothioate internucleotide linkages, and each other internucleotide linkage is a phosphodiester internucleotide linkage; and wherein the sense strand is 21 nucleotides in length and the nucleotides of the sense strand are modified such that, counting from the 5′ terminus of the sense strand, nucleotides 1, 3, 5, 7, 9, 11, 13, 15, 17, and 19 are 2′-fluoro nucleotides, nucleotides 2, 4, 6, 8, 10, 12, 14, 16, and 18 are 2′-O-methyl nucleotides, and nucleotides 20 and 21 are beta-D-deoxynucleotides, the first two internucleotide linkages at the 5′ terminus and the last two internucleotide linkages at the 3′ terminus are phosphorothioate internucleotide linkages, and each other internucleotide linkage is a phosphodiester internucleotide linkage. In such embodiments, the antisense strand has the modification pattern represented by Pattern I and the sense strand has the modification pattern represented by Pattern II.

In embodiments, a compound comprises an antisense strand and a sense strand hybridized to form a double-stranded nucleic acid, wherein the antisense strand and sense strand are not covalently linked (i.e. the antisense strand and sense strand form an siRNA), wherein the antisense strand is 19 nucleotides in length and the nucleotides of the antisense strand are modified such that, counting from the 5′ terminus of the antisense strand, nucleotides 1, 3, 5, 7, 9, 11, 13, 15, 17, and 19 are 2′-O-methyl nucleotides and nucleotides 2, 4, 6, 8, 10, 12, 14, 16, and 18 are 2′-fluoro nucleotides, the first two internucleotide linkages at the 5′ terminus and the last two internucleotide linkages at the 3′ terminus are phosphorothioate internucleotide linkages, and each other internucleotide linkage is a phosphodiester internucleotide linkage; and wherein the sense strand is 21 nucleotide in length and the nucleotides of the sense strand are modified such that, counting from the 5′ terminus of the sense strand, nucleotides 1, 3, 5, 7, 9, 11, 13, 15, 17, and 19 are 2′-fluoro nucleotides, nucleotides 2, 4, 6, 8, 10, 12, 14, 16, and 18 are 2′-O-methyl nucleotides, and nucleotides 20 and 21 are beta-D-deoxynucleotides, the first two internucleotide linkages at the 5′ terminus and the last two internucleotide linkages at the 3′ terminus are phosphorothioate internucleotide linkages, and each other internucleotide linkage is a phosphodiester internucleotide linkage. In such embodiments, the antisense strand has the modification pattern represented by Pattern III and the sense strand has the modification pattern represented by Pattern II.

In embodiments, a compound comprises an antisense strand and a sense strand hybridized to form a double-stranded nucleic acid, wherein the antisense strand and sense strand are not covalently linked (i.e. the antisense strand and sense strand form an siRNA), wherein the antisense strand is 21 nucleotides in length and the nucleotides of the antisense strand are modified such that, counting from the 5′ terminus of the antisense strand, nucleotides 1, 3, 5, 7, 9, 11, 13, 15, 17, and 19 are 2′-O-methyl nucleotides, nucleotides 2, 4, 6, 8, 10, 12, 14, 16, and 18 are 2′-fluoro nucleotides, and nucleotides 20 and 21 are beta-D-deoxy nucleotides, the first two internucleotide linkages at the 5′ terminus and the last two internucleotide linkages at the 3′ terminus are phosphorothioate internucleotide linkages, and each other internucleotide linkage is a phosphodiester internucleotide linkage; and wherein the sense strand is 19 nucleotides in length and the nucleotides of the sense strand are modified such that, counting from the 5′ terminus of the sense strand, nucleotides 1, 3, 5, 7, 9, 11, 13, 15, 17, and 19 are 2′-fluoro nucleotides and nucleotides 2, 4, 6, 8, 10, 12, 14, 16, and 18 are 2′-O-methyl nucleotides, the first two internucleotide linkages at the 5′ terminus and the last two internucleotide linkages at the 3′ terminus are phosphorothioate internucleotide linkages, and each other internucleotide linkage is a phosphodiester internucleotide linkage. In such embodiments, the antisense strand has the modification pattern represented by Pattern I and the sense strand has the modification pattern represented by Pattern IV.

In embodiments, a compound comprises an antisense strand and a sense strand hybridized to form a double-stranded nucleic acid, wherein the antisense strand and sense strand are not covalently linked (i.e. the antisense strand and sense strand form an siRNA), wherein the antisense strand is 19 nucleotides in length and the nucleotides of the antisense strand are modified such that, counting from the 5′ terminus of the antisense strand, nucleotides 1, 3, 5, 7, 9, 11, 13, 15, 17, and 19 are 2′-O-methyl nucleotides and nucleotides 2, 4, 6, 8, 10, 12, 14, 16, and 18 are 2′-fluoro nucleotides, the first two internucleotide linkages at the 5′ terminus and the last two internucleotide linkages at the 3′ terminus are phosphorothioate internucleotide linkages, and each other internucleotide is a phosphodiester internucleotide linkage; and wherein the sense strand is 19 nucleotides in length and the nucleotides of the sense strand are modified such that, counting from the 5′ terminus of the sense strand, nucleotides 1, 3, 5, 7, 9, 11, 13, 15, 17, and 19 are 2′-fluoro nucleotides and nucleotides 2, 4, 6, 8, 10, 12, 14, 16, and 18 are 2′-O-methyl nucleotides, the first two internucleotide linkages at the 5′ terminus and the last two internucleotide linkages at the 3′ terminus are phosphorothioate internucleotide linkages, and each other internucleotide linkage is a phosphodiester internucleotide linkage. In such embodiments, the antisense strand has the modification pattern represented by Pattern III and the sense strand has the modification pattern represented by Pattern IV.

In embodiments, a compound comprises an antisense strand and a sense strand hybridized to form a double-stranded nucleic acid, wherein the antisense strand and sense strand are not covalently linked (i.e. the antisense strand and sense strand form an siRNA), wherein the antisense strand is 23 nucleotides in length and wherein the nucleotides of the antisense strand are modified such that, counting from the 5′ terminus of the antisense strand, nucleotides 1, 3, 5, 7, 9, 11, 13, 15, 17, 19, 21, 22, and 23 are 2′-O-methyl nucleotides and nucleotides 2, 4, 6, 8, 10, 12, 14, 16, 18, and 20 are 2′-fluoro nucleotides the first two internucleotide linkages at the 5′ terminus and the last two internucleotide linkages at the 3′ terminus are phosphorothioate internucleotide linkages, and each other internucleotide linkage is a phosphodiester internucleotide linkage; and wherein the sense strand is 21 nucleotides in length and the nucleotides of the sense strand are modified such that, counting from the 5′ terminus of the sense strand, nucleotides 1, 3, 5, 7, 9, 11, 13, 15, 17, 19, and 21 are 2′-fluoro nucleotides, nucleotides 2, 4, 6, 8, 10, 12, 14, 16, 18, and 20 are 2′-O-methyl nucleotides, the first two internucleotide linkages at the 5′ terminus and the last two internucleotide linkages at the 3′ terminus are phosphorothioate internucleotide linkages, and each other internucleotide linkage is a phosphodiester internucleotide linkage. In such embodiments, the antisense strand has the modification pattern represented by Pattern V and the sense strand has the modification represented by Pattern VI.

In embodiments, a compound comprises an antisense strand and a sense strand hybridized to form a double-stranded nucleic acid, wherein the antisense strand and sense strand are not covalently linked (i.e. the antisense strand and sense strand form an siRNA), wherein the antisense strand is 23 nucleotides in length and wherein the nucleotides of the antisense strand are modified such that, counting from the 5′ terminus of the antisense strand, nucleotides 1, 3, 5, 7, 9, 11, 12, 13, 15, 17, 19, 21, 22, and 23 are 2′-O-methyl nucleotides and nucleotides 2, 4, 6, 8, 10, 14, 16, 18, and 20 are 2′-fluoro nucleotides, the first two internucleotide linkages at the 5′ terminus and the last two internucleotide linkages at the 3′ terminus are phosphorothioate internucleotide linkages, and each other internucleotide linkage is a phosphodiester internucleotide linkage; and wherein the sense strand is 21 nucleotides in length and the nucleotides of the sense strand are modified such that, counting from the 5′ terminus of the sense strand, nucleotides 1, 3, 5, 7, 9, 10, 11, 13, 15, 17, 19, and 21 are 2′-fluoronucleotides, nucleotides 2, 4, 6, 8, 12, 14, 16, 18, and 20 are 2′-O-methyl nucleotides, the first two internucleotide linkages at the 5′ terminus and the last two internucleotide linkages at the 3′ terminus are phosphorothioate internucleotide linkages, and each other internucleotide linkage is a phosphodiester internucleotide linkage. In such embodiments, the antisense strand has the modification pattern represented by Pattern VII and the sense strand has the modification pattern represented by Pattern VIII.

In embodiments, a compound comprises an antisense strand and a sense strand hybridized to form a double-stranded nucleic acid, wherein the antisense strand and sense strand are not covalently linked (i.e. the antisense strand and sense strand form an siRNA), wherein the antisense strand is 23 nucleotides in length and wherein the nucleotides of the antisense strand are modified such that, counting from the 5′ terminus of the antisense strand, nucleotides 1, 3, 5, 7, 9, 10, 11, 13, 15, 17, 19, 21, 22, and 23 are 2′-O-methyl nucleotides and nucleotides 2, 4, 6, 8, 12, 14, 16, 18, and 20 are 2′-fluoro nucleotides, the first two internucleotide linkages at the 5′ terminus and the last two internucleotide linkages at the 3′ terminus are phosphorothioate internucleotide linkages, and each other internucleotide linkage is a phosphodiester internucleotide linkage; and wherein the sense strand is 21 nucleotides in length and nucleotides of the sense strand are modified such that, counting from the 5′ terminus of the sense strand, nucleotides 1, 3, 5, 7, 9, 11, 12, 13, 15, 17, 19, and 21 are 2′-fluoro nucleotides, nucleotides 2, 4, 6, 8, 10, 14, 16, 18, and 20 are 2′-O-methyl nucleotides, the first two internucleotide linkages at the 5′ terminus and the last two internucleotide linkages at the 3′ terminus are phosphorothioate internucleotide linkages, and each other internucleotide linkage is a phosphodiester internucleotide linkage. In such embodiments, the antisense strand has the modification pattern of Pattern IX and the sense strand has the modification pattern of Pattern X.

In embodiments, a compound comprises an antisense strand and a sense strand hybridized to form a double-stranded nucleic acid, wherein the antisense strand and sense strand are not covalently linked (i.e. the antisense strand and sense strand form an siRNA), wherein the antisense strand is 23 nucleotides in length and wherein the nucleotides of the antisense strand are modified such that, counting from the 5′ terminus of the antisense strand, nucleotides 1, 3, 5, 7, 9, 11, 13, 15, 17, 19, 21, 22, and 23 are 2′-O-methyl nucleotides and nucleotides 2, 4, 6, 8, 10, 12, 14, 16, 18, and 20 are 2′-fluoro nucleotides the first two internucleotide linkages at the 5′ terminus and the last two internucleotide linkages at the 3′ terminus are phosphorothioate internucleotide linkages, and each other internucleotide linkage is a phosphodiester internucleotide linkage; and wherein the sense strand is 23 nucleotides in length and wherein the nucleotides of the sense strand are modified such that, counting from the 5′ terminus of the sense strand, nucleotides 1, 3, 5, 7, 9, 11, 13, 15, 17, 19, and 21 are 2′-fluoro nucleotides, nucleotides 2, 4, 6, 8, 10, 12, 14, 16, 18, and 20 are 2′-O-methyl nucleotides, nucleotides 22 and 23 are beta-D-deoxynucleotides, the first two internucleotide linkages at the 5′ terminus and the last two internucleotide linkages at the 3′ terminus are phosphorothioate internucleotide linkages, and each other internucleotide linkage is a phosphodiester internucleotide linkage. In such embodiments, the antisense strand has the modification pattern represented by Pattern V and the sense strand has the modification represented by Pattern XI.

In embodiments, a compound comprises an antisense strand and a sense strand hybridized to form a double-stranded nucleic acid, wherein the antisense strand and sense strand are not covalently linked (i.e. the antisense strand and sense strand form an siRNA), wherein the antisense strand is 23 nucleotides in length and wherein the nucleotides of the antisense strand are modified such that, counting from the 5′ terminus of the antisense strand, nucleotides 1, 3, 5, 7, 9, 11, 12, 13, 15, 17, 19, 21, 22, and 23 are 2′-O-methyl nucleotides and nucleotides 2, 4, 6, 8, 10, 14, 16, 18, and 20 are 2′-fluoro nucleotides, the first two internucleotide linkages at the 5′ terminus and the last two internucleotide linkages at the 3′ terminus are phosphorothioate internucleotide linkages, and each other internucleotide linkage is a phosphodiester internucleotide linkage; and wherein the sense strand is 23 nucleotides in length and wherein the nucleotides of the sense strand are modified such that, counting from the 5′ terminus of the sense strand, nucleotides 1, 3, 5, 7, 9, 10, 11, 13, 15, 17, 19, and 21 are 2′-fluoro nucleotides, nucleotides 2, 4, 6, 8, 12, 14, 16, 18, and 20 are 2′-O-methyl nucleotides, nucleotides 22 and 23 are beta-D-deoxynucleotides, the first two internucleotide linkages at the 5′ terminus and the last two internucleotide linkages at the 3′ terminus are phosphorothioate internucleotide linkages, and each other internucleotide linkage is a phosphodiester internucleotide linkage. In such embodiments, the antisense strand has the modification pattern represented by Pattern VII and the sense strand has the modification pattern represented by Pattern XII.

In embodiments, a compound comprises an antisense strand and a sense strand hybridized to form a double-stranded nucleic acid, wherein the antisense strand and the sense strand are not covalently linked (i.e. the antisense strand and sense strand form an siRNA), wherein the antisense strand is 23 nucleotides in length and wherein the nucleotides of the antisense strand are modified such, that counting from the 5′ terminus of the antisense strand, nucleotides 1, 3, 5, 7, 9, 10, 11, 13, 15, 17, 19, 21, 22, and 23 are 2′-O-methyl nucleotides and nucleotides 2, 4, 6, 8, 12, 14, 16, 18, and 20 are 2′-fluoro nucleotides, the first two internucleotide linkages at the 5′ terminus and the last two internucleotide linkages at the 3′ terminus are phosphorothioate internucleotide linkages, and each other internucleotide linkage is a phosphodiester internucleotide linkage; and wherein the sense strand is 23 nucleotides in length and wherein the nucleotides of the sense strand are modified such that, counting from the 5′ terminus of the sense strand, nucleotides 1, 3, 5, 7, 9, 11, 12, 13, 15, 17, 19, and 21 are 2′-fluoro nucleotides, nucleotides 2, 4, 6, 8, 10, 14, 16, 18, and 20 are 2′-O-methyl nucleotides, nucleotides 22 and 23 are beta-D-deoxynucleotides, the first two internucleotide linkages at the 5′ terminus and the last two internucleotide linkages at the 3′ terminus are phosphorothioate internucleotide linkages, and each other internucleotide linkage is a phosphodiester internucleotide linkage. In such embodiments, the antisense strand has the modification pattern of Pattern IX and the sense strand has the modification pattern of Pattern XIII.

In embodiments, a compound comprises an antisense strand and a sense strand hybridized to form a double-stranded nucleic acid, wherein the antisense strand and the sense strand are not covalently linked (i.e., the antisense strand and sense strand from an siRNA), wherein the antisense strand is 23 nucleotides in length and wherein the nucleotides of the antisense strand are modified such that, counting from the 5′ terminus of the antisense strand, nucleotides 1, 3, 5, 7, 9, 11, 13, 15, 17, 19, 21, 22, and 23 are 2′-O-methyl nucleotides, nucleotides 2, 4, 6, 8, 10, 12, 14, 16, 18, and 20 are 2′-fluoro nucleotides, the first two internucleotide linkages at the 5′ terminus and the last two internucleotide linkages at the 3′ terminus are phosphorothioate internucleotide linkages, and each other internucleotide linkage is a phosphodiester internucleotide linkage; and wherein the sense strand is 21 nucleotides in length and wherein the nucleotides of the sense strand are modified such that, counting from the 5′ terminus of the sense strand, nucleotides 1, 2, 3, 4, 6, 8, 12, 14, 16, 18, 19, 20, and 21 are 2′-O-methyl nucleotides, nucleotides 5, 7, 9, 10, 11, 13, 15, and 17 are 2′-fluoro nucleotides, the first two internucleotide linkages at the 5′ terminus and the last two internucleotide linkages at the 3′ terminus are phosphorothioate internucleotide linkages, and each other internucleotide linkage is a phosphodiester internucleotide linkage. In such embodiments, the antisense strand has the modification pattern of Pattern V and the sense strand has the modification pattern of Pattern XIV.

In embodiments, a compound comprises an antisense strand and a sense strand hybridized to form a double-stranded nucleic acid, wherein the antisense strand and the sense strand are not covalently linked (i.e., the antisense strand and sense strand from an siRNA), wherein the antisense strand is 23 nucleotides in length and the nucleotides of the antisense strand are modified such that, counting from the 5′ terminus of the antisense strand, nucleotides 1, 3, 5, 7, 8, 9, 11, 12, 13, 15, 17, 19, 21, 22, and 23 are 2′-O-methyl nucleotides, nucleotides 2, 4, 6, 10, 14, 16, 18, and 20 are 2′-fluoro nucleotides, the first two internucleotide linkages at the 5′ terminus and the last two internucleotide linkages at the 3′ terminus are phosphorothioate internucleotide linkages, and each other internucleotide linkage is a phosphodiester internucleotide linkage; and wherein the sense strand is 21 nucleotides in length and the nucleotides of the sense strand are modified such that, counting from the 5′ terminus of the sense strand, nucleotides 1, 2, 3, 4, 5, 6, 8, 12, 14, 16, 18, 19, 20, and 21 are 2′-O-methyl nucleotides, nucleotides 7, 9, 10, 11, 13, 15, and 17 are 2′-fluoro nucleotides, the first two internucleotide linkages at the 5′ terminus and the last two internucleotide linkages at the 3′ terminus are phosphorothioate internucleotide linkages, and each other internucleotide linkage is a phosphodiester internucleotide linkage. In such embodiments, the antisense strand has the modification pattern of Pattern XVI and the sense strand has the modification pattern of Pattern XV.

In embodiments, a compound comprises an antisense strand and a sense strand hybridized to form a double-stranded nucleic acid, wherein the antisense strand and the sense strand are not covalently linked (i.e., the antisense strand and sense strand from an siRNA), wherein the antisense strand is 23 nucleotides in length and the nucleotides of the antisense strand are modified such that, counting from the 5′ terminus of the antisense strand, nucleotides 1, 3, 5, 7, 8, 9, 11, 12, 13, 15, 17, 19, 21, 22, and 23 are 2′-O-methyl nucleotides, nucleotides 2, 4, 6, 10, 14, 16, 18, and 20 are 2′-fluoro nucleotides, the first two internucleotide linkages at the 5′ terminus and the last two internucleotide linkages at the 3′ terminus are phosphorothioate internucleotide linkages, and each other internucleotide linkage is a phosphodiester internucleotide linkage; and wherein the sense strand is 21 nucleotides in length and the nucleotides of the sense strand are modified such that, counting from the 5′ terminus of the sense strand, nucleotides 1, 2, 3, 4, 5, 6, 8, 12, 13, 14, 15, 16, 17, 18, 19, 20, and 21 are 2′-O-methyl nucleotides, nucleotides 7, 9, 10, and 11 are 2′-fluoro nucleotides, the first two internucleotide linkages at the 5′ terminus and the last two internucleotide linkages at the 3′ terminus are phosphorothioate internucleotide linkages, and each other internucleotide linkage is a phosphodiester internucleotide linkage. In such embodiments, the antisense strand has the modification pattern of Pattern XVII and the sense strand has the modification pattern of Pattern XVIII.

In embodiments, a compound comprises an antisense strand and a sense strand hybridized to form a double-stranded nucleic acid, wherein the antisense strand and the sense strand are not covalently linked (i.e., the antisense strand and sense strand from an siRNA), wherein the antisense strand is 23 nucleotides in length and the nucleotides of the antisense strand are modified such that, counting from the 5′ terminus of the antisense strand, nucleotides 1, 3, 5, 7, 8, 9, 11, 12, 13, 15, 17, 19, 21, 22, and 23 are 2′-O-methyl nucleotides, nucleotides 2, 4, 6, 10, 14, 16, 18, and 20 are 2′-fluoro nucleotides, the first two internucleotide linkages at the 5′ terminus and the last two internucleotide linkages at the 3′ terminus are phosphorothioate internucleotide linkages, and each other internucleotide linkage is a phosphodiester internucleotide linkage; and wherein the sense strand is 21 nucleotides in length and the nucleotides of the sense strand are modified such that, counting from the 5′ terminus of the sense strand, nucleotides 1, 2, 3, 4, 5, 6, 8, 12, 13, 14, 15, 16, 17, 18, 19, 20, and 21 are 2′-O-methyl nucleotides, nucleotides 7, 9, 10, and 11 are 2′-fluoro nucleotides, the first two internucleotide linkages at the 5′ terminus are phosphorothioate internucleotide linkages, and each other internucleotide linkage is a phosphodiester internucleotide linkage. In such embodiments, the antisense strand has the modification pattern of Pattern XVII and the sense strand has the modification pattern of Pattern XIX.

In embodiments, a compound comprises an antisense strand and a sense strand hybridized to form a double-stranded nucleic acid, wherein the antisense strand and the sense strand are not covalently linked (i.e., the antisense strand and sense strand from an siRNA), wherein the antisense strand is 23 nucleotides in length and wherein the nucleotides of the antisense strand are modified such that, counting from the 5′ terminus of the antisense strand, nucleotides 1, 3, 5, 7, 9, 11, 13, 15, 17, 19, 21, 22, and 23 are 2′-O-methyl nucleotides, nucleotides 2, 4, 6, 8, 10, 12, 14, 16, 18, and 20 are 2′-fluoro nucleotides, the first two internucleotide linkages at the 5′ terminus and the last two internucleotide linkages at the 3′ terminus are phosphorothioate internucleotide linkages, and each other internucleotide linkage is a phosphodiester internucleotide linkage; and wherein the sense strand is 21 nucleotides in length and wherein the nucleotides of the sense strand are modified such that, counting from the 5′ terminus of the sense strand, nucleotides 1 and 2 are 2′-O-methoxyethyl nucleotides, nucleotides 3, 4, 6, 8, 12, 14, 16, 18, 19, 20, and 21 are 2′-O-methyl nucleotides, nucleotides 5, 7, 9, 10, 11, 13, 15, and 17 are 2′-fluoro nucleotides, the first two internucleotide linkages at the 5′ terminus and the last two internucleotide linkages at the 3′ terminus are phosphorothioate internucleotide linkages, and each other internucleotide linkage is a phosphodiester internucleotide linkage. In such embodiments, the antisense strand has the modification pattern of Pattern V and the sense strand has the modification pattern of Pattern XX.

In embodiments, a compound comprises an antisense strand and a sense strand hybridized to form a double-stranded nucleic acid, wherein the antisense strand and the sense strand are not covalently linked (i.e., the antisense strand and sense strand from an siRNA), wherein the antisense strand is 23 nucleotides in length and wherein the nucleotides of the antisense strand are modified such that, counting from the 5′ terminus of the antisense strand, nucleotides 1, 3, 5, 7, 9, 11, 13, 15, 17, 19, 21, 22, and 23 are 2′-O-methyl nucleotides, nucleotides 2, 4, 6, 8, 10, 12, 14, 16, 18, and 20 are 2′-fluoro nucleotides, the first two internucleotide linkages at the 5′ terminus and the last two internucleotide linkages at the 3′ terminus are phosphorothioate internucleotide linkages, and each other internucleotide linkage is a phosphodiester internucleotide linkage; and wherein the sense strand is 21 nucleotides in length and wherein the nucleotides of the sense strand are modified such that, counting from the 5′ terminus of the sense strand, nucleotides 2 and 3 are 2′-O-methoxyethyl nucleotides, nucleotides 1, 4, 6, 8, 12, 14, 16, 18, 19, 20, and 21 are 2′-O-methyl nucleotides, nucleotides 5, 7, 9, 10, 11, 13, 15, and 17 are 2′-fluoro nucleotides, the first two internucleotide linkages at the 5′ terminus and the last two internucleotide linkages at the 3′ terminus are phosphorothioate internucleotide linkages, and each other internucleotide linkage is a phosphodiester internucleotide linkage. In such embodiments, the antisense strand has the modification pattern of Pattern V and the sense strand has the modification pattern of Pattern XXI.

In embodiments, a compound comprises an antisense strand and a sense strand hybridized to form a double-stranded nucleic acid, wherein the antisense strand and the sense strand are not covalently linked (i.e., the antisense strand and sense strand from an siRNA), wherein the antisense strand is 23 nucleotides in length and wherein the nucleotides of the antisense strand are modified such that, counting from the 5′ terminus of the antisense strand, nucleotides 1, 3, 5, 7, 9, 11, 13, 15, 17, 19, 21, 22, and 23 are 2′-O-methyl nucleotides, nucleotides 2, 4, 6, 8, 10, 12, 14, 16, 18, and 20 are 2′-fluoro nucleotides, the first two internucleotide linkages at the 5′ terminus and the last two internucleotide linkages at the 3′ terminus are phosphorothioate internucleotide linkages, and each other internucleotide linkage is a phosphodiester internucleotide linkage; and wherein the sense strand is 21 nucleotides in length and wherein the nucleotides of the sense strand are modified such that, counting from the 5′ terminus of the sense strand, nucleotides 2, 3, 19 and 20 are 2′-O-methoxyethyl nucleotides, nucleotides 1, 4, 6, 8, 12, 14, 16, 18, and 21 are 2′-O-methyl nucleotides, nucleotides 5, 7, 9, 10, 11, 13, 15, and 17 are 2′-fluoro nucleotides, the first two internucleotide linkages at the 5′ terminus and the last two internucleotide linkages at the 3′ terminus are phosphorothioate internucleotide linkages, and each other internucleotide linkage is a phosphodiester internucleotide linkage. In such embodiments, the antisense strand has the modification pattern of Pattern V and the sense strand has the modification pattern of Pattern XXII.

In embodiments, a compound comprises an antisense strand and a sense strand hybridized to form a double-stranded nucleic acid, wherein the antisense strand and the sense strand are not covalently linked (i.e., the antisense strand and sense strand from an siRNA), wherein the antisense strand is 23 nucleotides in length and wherein the nucleotides of the antisense strand are modified such that, counting from the 5′ terminus of the antisense strand, nucleotides 1, 3, 5, 7, 9, 11, 13, 15, 17, 19, 21, 22, and 23 are 2′-O-methyl nucleotides, nucleotides 2, 4, 6, 8, 10, 12, 14, 16, 18, and 20 are 2′-fluoro nucleotides, the first two internucleotide linkages at the 5′ terminus and the last two internucleotide linkages at the 3′ terminus are phosphorothioate internucleotide linkages, and each other internucleotide linkage is a phosphodiester internucleotide linkage; and wherein the sense strand is 21 nucleotides in length and wherein the nucleotides of the sense strand are modified such that, counting from the 5′ terminus of the sense strand, nucleotides 1, 2, 3, and 4 are 2′-O-methoxyethyl nucleotides, nucleotides 6, 8, 12, 14, 16, 18, 19, 20 and 21 are 2′-O-methyl nucleotides, nucleotides 5, 7, 9, 10, 11, 13, 15, and 17 are 2′-fluoro nucleotides, the first two internucleotide linkages at the 5′ terminus and the last two internucleotide linkages at the 3′ terminus are phosphorothioate internucleotide linkages, and each other internucleotide linkage is a phosphodiester internucleotide linkage. In such embodiments, the antisense strand has the modification pattern of Pattern V and the sense strand has the modification pattern of Pattern XXIV.

### Conjugated Compounds

In embodiments, a compound provided herein comprises a covalently linked ligand. In embodiments, a compound provided herein comprises a ligand covalently linked to the antisense strand. In embodiments, a compound provided herein comprises a ligand covalently linked to the sense strand. In embodiments, the ligand comprises an uptake motif with one or more long chain fatty acids (LFCA).

In embodiments, a compound comprising an uptake motif has the structure (I)

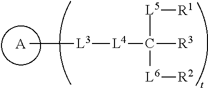

wherein A is a double-stranded nucleic acid and t is an integer from 1 to 5. In embodiments, A is the sense strand. In embodiments, A is the antisense strand.

L³ and L⁴ are independently a bond, —N(R²³)—, —O—, —S—, —C(O)—, —N(R²³)C(O)—, —C(O)N(R²⁴)—, —N(R²³)C(O)N(R²⁴)—, —C(O)O—, —OC(O)—, —N(R²³)C(O)O—, —OC(O)N(R²⁴)—, —OPO₂—O—, —O—P(O)(S)—O—, —O—P(O)(R²⁵)—O—, —O—P(S)(R²⁵)—O—, —O—P(O)(NR²³R²⁴)—N—, —O—P(S)(NR²³R²⁴)—N—, —O—P(O)(NR²³R²⁴)—O—, —O—P(S)(NR²³R²⁴)—O—, —P(O)(NR²³R²⁴)—N—, —P(S)(NR²³R²⁴)—N—, —P(O)(NR²³R²⁴)—O—, —P(S)(NR²³R²⁴)—O—, —S—S—, substituted or unsubstituted alkylene, substituted or unsubstituted heteroalkylene, substituted or unsubstituted cycloalkylene, substituted or unsubstituted heterocycloalkylene, substituted or unsubstituted arylene or substituted or unsubstituted heteroarylene. Each R²³, R²⁴ and R²⁵ is independently hydrogen or unsubstituted C₁-C₁₀ alkyl.

L^{S} is -L^{5A}-L^{5B}-L^{5C}-L^{5D}-L^{5E}- and L⁶ is -L^{6A}-L^{6B}-L^{6C}-L^{6D}-L^{6E}-L^{5A}, L^{5B}, L^{5C}, L^{5D}, L^{5E}, L^{6A}, L^{6B}, L^{6C}, L^{6D}, and L^{6E} are independently a bond, —NH—, —O—, —S—, —C(O)—, —NHC(O)—, —NHC(O)NH—, —C(O)O—, —OC(O)—, —C(O)NH—, substituted or unsubstituted alkylene, substituted or unsubstituted heteroalkylene, substituted or unsubstituted cycloalkylene, substituted or unsubstituted heterocycloalkylene, substituted or unsubstituted arylene or substituted or unsubstituted heteroarylene.

R¹ and R² are independently unsubstituted C₁-C₂₅ alkyl, wherein at least one of R¹ and R² is unsubstituted C₉-C₁₉ alkyl. In embodiments, R¹ and R² are independently unsubstituted C₁-C₂₀ alkyl, wherein at least one of R¹ and R² is unsubstituted C₉-C₁₉ alkyl.

R³ is hydrogen, -hydrogen, —NH₂, —OH, —SH, —C(O)H, —C(O)NH₂, —NHC(O)H, —NHC(O)OH, —NHC(O)NH₂, —C(O)OH, —OC(O)H, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In embodiments, t is 1. In embodiments, t is 2. In embodiments, t is 3. In embodiments, t is 4. In embodiments, t is 5.

In embodiments, one L³ is attached to a 3′ carbon of a nucleotide. In embodiments, one L³ is attached to the 3′ carbon the 3′ terminal nucleotide of the sense strand. In embodiments, one L³ is attached to the 3′ carbon of the 3′ terminal nucleotide of the antisense strand.

In embodiments, one L³ is attached to a 5′ carbon of a nucleotide. In embodiments, one L³ is attached to the 5′ carbon of the 5′ terminal nucleotide of the sense strand. In embodiments, one L³ is attached to the 5′ carbon of the 5′ terminal nucleotide of the antisense strand.

In embodiments, one L³ is attached to a 2′ carbon of a nucleotide. In embodiments, one L³ is attached to a 2′ carbon of a nucleotide of the sense strand. In embodiments, one L³ is attached to a 2′ carbon of a nucleotide of the antisense strand.

In embodiments, one L³ is attached to a nucleobase. In embodiments, one L³ is attached to a nucleobase of the sense strand. In embodiments, one L³ is attached to a nucleobase of the antisense strand.

In embodiments, one L³ is attached to a phosphate group at a 3′ carbon of a nucleotide. In embodiments, one L³ is attached to a phosphate group at the 3′ carbon the 3′ terminal nucleotide of the sense strand. In embodiments, one L³ is attached to a phosphate group at the 3′ carbon of the 3′ terminal nucleotide of the antisense strand.

In embodiments, one L³ is attached to a phosphate group at a 5′ carbon of a nucleotide. In embodiments, one L³ is attached to a phosphate group at the 5′ carbon of the 5′ terminal nucleotide of the sense strand. In embodiments, one L³ is attached to a phosphate group at the 5′ carbon of the 5′ terminal nucleotide of the antisense strand.

In embodiments, one L³ is attached to a phosphate group at a 2′ carbon of a nucleotide. In embodiments, one L³ is attached to a phosphate group at a 2′ carbon of a nucleotide of the sense strand. In embodiments, one L³ is attached to a phosphate group a 2′ carbon of a nucleotide of the antisense strand.

In embodiments, L³ is a bond, —N(R²³)—, —O—, —S—, —C(O)—, —N(R²³)C(O)—, —C(O)N(R²⁴)—, —N(R²³)C(O)N(R²⁴)—, —C(O)O—, —OC(O)—, —N(R²³)C(O)O—, —OC(O)N(R²⁴)—, —OPO₂—O—, —O—P(O)(S)—O—, —O—P(O)(R²⁵)—O—, —O—P(S)(R²⁵)—O—, —O—P(O)(NR²³R²⁴)—N—, —O—P(S)(NR²³R²⁴)—N—, —O—P(O)(NR²³R²⁴)—O—, —O—P(S)(NR²³R²⁴)—O—, —P(O)(NR²³R²⁴)—N—, —P(S)(NR²³R²⁴)—N—, —P(O)(NR²³R²⁴)—O—, —P(S)(NR²³R²⁴)—O—, —S—S—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene.

In embodiments, L³ is a bond. In embodiments, L³ is —N(R²³)—. In embodiments, L³ is —O— or —S—. In embodiments, L³ is —C(O)—. In embodiments, L³ is —N(R²³)C(O)— or —C(O)N(R²⁴)—. In embodiments, L³ is —N(R²³)C(O)N(R²⁴)—. In embodiments, L³ is —C(O)O— or —OC(O)—. In embodiments, L³ is —N(R²³)C(O)O— or —OC(O)N(R²⁴)—. In embodiments, L³ is —OPO₂—O—, —O—P(O)(S)—O—, —O—P(O)(R²⁵)—O—, —O—P(O)(NR²³R²⁴)—N—, or —O—P(O)(NR²³R²⁴)—O—. In embodiments, L³ is —P(O)(NR²³R²⁴)—N—, —P(S)(NR²³R²⁴)—N—, —P(O)(NR²³R²⁴)—O— or —P(S)(NR²³R²⁴)—O—. In embodiments, L³ is —S—S—.

In embodiments, L³ is independently substituted or unsubstituted alkylene (e.g., C₁-C₂₃, C₁-C₁₂, C₁-C₈, C₁-C₆, C₁-C₄, or C₁-C₂). In embodiments, L³ is independently substituted alkylene (e.g., C₁-C₂₃, C₁-C₁₂, C₁-C₈, C₁-C₆, C₁-C₄, or C₁-C₂). In embodiments, L³ is independently unsubstituted alkylene (e.g., C₁-C₂₃, C₁-C₁₂, C₁-C₈, C₁-C₆, C₁-C₄, or C₁-C₂). In embodiments, L³ is independently substituted or unsubstituted C₁-C₂₃ alkylene. In embodiments, L³ is independently substituted C₁-C₂₃ alkylene. In embodiments, L³ is independently unsubstituted C₁-C₂₃ alkylene. In embodiments, L³ is independently substituted or unsubstituted C₁-C₁₂ alkylene. In embodiments, L³ is independently substituted C₁-C₁₂ alkylene. In embodiments, L³ is independently unsubstituted C₁-C₁₂ alkylene. In embodiments, L³ is independently substituted or unsubstituted C₁-C₈ alkylene. In embodiments, L³ is independently substituted C₁-C₈ alkylene. In embodiments, L³ is independently unsubstituted C₁-C₈ alkylene. In embodiments, L³ is independently substituted or unsubstituted C₁-C₆ alkylene. In embodiments, L³ is independently substituted C₁-C₆ alkylene. In embodiments, L³ is independently unsubstituted C₁-C₆ alkylene. In embodiments, L³ is independently substituted or unsubstituted C₁-C₄ alkylene. In embodiments, L³ is independently substituted C₁-C₄ alkylene. In embodiments, L³ is independently unsubstituted C₁-C₄ alkylene. In embodiments, L³ is independently substituted or unsubstituted ethylene. In embodiments, L³ is independently substituted ethylene. In embodiments, L³ is independently unsubstituted ethylene. In embodiments, L³ is independently substituted or unsubstituted methylene. In embodiments, L³ is independently substituted methylene. In embodiments, L³ is independently unsubstituted methylene.

In embodiments, L³ is independently substituted or unsubstituted heteroalkylene (e.g., 2 to 23 membered, 2 to 12 membered, 2 to 8 membered, 2 to 6 membered, 4 to 6 membered, 2 to 3 membered, or 4 to 5 membered). In embodiments, L³ is independently substituted heteroalkylene (e.g., 2 to 23 membered, 2 to 12 membered, 2 to 8 membered, 2 to 6 membered, 4 to 6 membered, 2 to 3 membered, or 4 to 5 membered). In embodiments, L³ is independently unsubstituted heteroalkylene (e.g., 2 to 23 membered, 2 to 12 membered, 2 to 8 membered, 2 to 6 membered, 4 to 6 membered, 2 to 3 membered, or 4 to 5 membered). In embodiments, L³ is independently substituted or unsubstituted 2 to 23 membered heteroalkylene. In embodiments, L³ is independently substituted 2 to 23 membered heteroalkylene. In embodiments, L³ is independently unsubstituted 2 to 23 membered heteroalkylene. In embodiments, L³ is independently substituted or unsubstituted 2 to 8 membered heteroalkylene. In embodiments, L³ is independently substituted 2 to 8 membered heteroalkylene. In embodiments, L³ is independently unsubstituted 2 to 8 membered heteroalkylene. In embodiments, L³ is independently substituted or unsubstituted 2 to 6 membered heteroalkylene. In embodiments, L³ is independently substituted 2 to 6 membered heteroalkylene. In embodiments, L³ is independently unsubstituted 2 to 6 membered heteroalkylene. In embodiments, L³ is independently substituted or unsubstituted 4 to 6 membered heteroalkylene. In embodiments, L³ is independently substituted 4 to 6 membered heteroalkylene. In embodiments, L³ is independently unsubstituted 4 to 6 membered heteroalkylene. In embodiments, L³ is independently substituted or unsubstituted 2 to 3 membered heteroalkylene. In embodiments, L³ is independently substituted 2 to 3 membered heteroalkylene. In embodiments, L³ is independently unsubstituted 2 to 3 membered heteroalkylene. In embodiments, L³ is independently substituted or unsubstituted 4 to 5 membered heteroalkylene. In embodiments, L³ is independently substituted 4 to 5 membered heteroalkylene. In embodiments, L³ is independently unsubstituted 4 to 5 membered heteroalkylene.

In embodiments, L⁴ is a bond, —N(R²³)—, —O—, —S—, —C(O)—, —N(R²³)C(O)—, —C(O)N(R²⁴)—, —N(R²³)C(O)N(R²⁴)—, —C(O)O—, —OC(O)—, —N(R²³)C(O)O—, —OC(O)N(R²⁴)—, —OPO₂—O—, —O—P(O)(S)—O—, —O—P(O)(R²⁵)—O—, —O—P(S)(R²⁵)—O—, —O—P(O)(NR²³R²⁴)—N—, —O—P(S)(NR²³R²⁴)—N—, —O—P(O)(NR²³R²⁴)—O—, —O—P(S)(NR²³R²⁴)—O—, —P(O)(NR²³R²⁴)—N—, —P(S)(NR²³R²⁴)—N—, —P(O)(NR²³R²⁴)—O—, —P(S)(NR²³R²⁴)—O—, —S—S—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene.

In embodiments, L⁴ is a bond. In embodiments, L⁴ is —N(R²³)—. In embodiments, L⁴ is —O— or —S—. In embodiments, L⁴ is —C(O)—. In embodiments, L⁴ is —N(R²³)C(O)— or —C(O)N(R²⁴)—. In embodiments, L⁴ is —N(R²³)C(O)N(R²⁴)—. In embodiments, L⁴ is —C(O)O— or —OC(O)—. In embodiments, L⁴ is —N(R²³)C(O)O— or —OC(O)N(R²⁴)—. In embodiments, L⁴ is —OPO₂—O—, —O—P(O)(S)—O—, —O—P(O)(R²⁵)—O—, —O—P(O)(NR²³R²⁴)—N—, or —O—P(O)(NR²³R²⁴)—O—. In embodiments, L⁴ is —P(O)(NR²³R²⁴)—N—, —P(S)(NR²³R²⁴)—N—, —P(O)(NR²³R²⁴)—O— or —P(S)(NR²³R²⁴)—O—. In embodiments, L⁴ is —S—S—.

In embodiments, L⁴ is independently substituted or unsubstituted alkylene (e.g., C₁-C₂₃, C₁-C₁₂, C₁-C₈, C₁-C₆, C₁-C₄, or C₁-C₂). In embodiments, L⁴ is independently substituted alkylene (e.g., C₁-C₂₃, C₁-C₁₂, C₁-C₈, C₁-C₆, C₁-C₄, or C₁-C₂). In embodiments, L⁴ is independently unsubstituted alkylene (e.g., C₁-C₂₃, C₁-C₁₂, C₁-C₈, C₁-C₆, C₁-C₄, or C₁-C₂). In embodiments, L⁴ is independently substituted or unsubstituted C₁-C₂₃ alkylene. In embodiments, L⁴ is independently substituted C₁-C₂₃ alkylene. In embodiments, L⁴ is independently unsubstituted C₁-C₂₃ alkylene. In embodiments, L⁴ is independently substituted or unsubstituted C₁-C₁₂ alkylene. In embodiments, L⁴ is independently substituted C₁-C₁₂ alkylene. In embodiments, L⁴ is independently unsubstituted C₁-C₁₂ alkylene. In embodiments, L⁴ is independently substituted or unsubstituted C₁-C₈ alkylene. In embodiments, L⁴ is independently substituted C₁-C₈ alkylene. In embodiments, L⁴ is independently unsubstituted C₁-C₈ alkylene. In embodiments, L⁴ is independently substituted or unsubstituted C₁-C₆ alkylene. In embodiments, L⁴ is independently substituted C₁-C₆ alkylene. In embodiments, L⁴ is independently unsubstituted C₁-C₆ alkylene. In embodiments, L⁴ is independently substituted or unsubstituted C₁-C₄ alkylene. In embodiments, L⁴ is independently substituted C₁-C₄ alkylene. In embodiments, L⁴ is independently unsubstituted C₁-C₄ alkylene. In embodiments, L⁴ is independently substituted or unsubstituted ethylene. In embodiments, L⁴ is independently substituted ethylene. In embodiments, L⁴ is independently unsubstituted ethylene. In embodiments, L⁴ is independently substituted or unsubstituted methylene. In embodiments, L⁴ is independently substituted methylene. In embodiments, L⁴ is independently unsubstituted methylene.

In embodiments, L⁴ is independently substituted or unsubstituted heteroalkylene (e.g., 2 to 23 membered, 2 to 12 membered, 2 to 8 membered, 2 to 6 membered, 4 to 6 membered, 2 to 3 membered, or 4 to 5 membered). In embodiments, L⁴ is independently substituted heteroalkylene (e.g., 2 to 23 membered, 2 to 12 membered, 2 to 8 membered, 2 to 6 membered, 4 to 6 membered, 2 to 3 membered, or 4 to 5 membered). In embodiments, L⁴ is independently unsubstituted heteroalkylene (e.g., 2 to 23 membered, 2 to 12 membered, 2 to 8 membered, 2 to 6 membered, 4 to 6 membered, 2 to 3 membered, or 4 to 5 membered). In embodiments, L⁴ is independently substituted or unsubstituted 2 to 23 membered heteroalkylene. In embodiments, L⁴ is independently substituted 2 to 23 membered heteroalkylene. In embodiments, L⁴ is independently unsubstituted 2 to 23 membered heteroalkylene. In embodiments, L⁴ is independently substituted or unsubstituted 2 to 8 membered heteroalkylene. In embodiments, L⁴ is independently substituted 2 to 8 membered heteroalkylene. In embodiments, L⁴ is independently unsubstituted 2 to 8 membered heteroalkylene. In embodiments, L⁴ is independently substituted or unsubstituted 2 to 6 membered heteroalkylene. In embodiments, L⁴ is independently substituted 2 to 6 membered heteroalkylene. In embodiments, L⁴ is independently unsubstituted 2 to 6 membered heteroalkylene. In embodiments, L⁴ is independently substituted or unsubstituted 4 to 6 membered heteroalkylene. In embodiments, L⁴ is independently substituted 4 to 6 membered heteroalkylene. In embodiments, L⁴ is independently unsubstituted 4 to 6 membered heteroalkylene. In embodiments, L⁴ is independently substituted or unsubstituted 2 to 3 membered heteroalkylene. In embodiments, L⁴ is independently substituted 2 to 3 membered heteroalkylene. In embodiments, L⁴ is independently unsubstituted 2 to 3 membered heteroalkylene. In embodiments, L⁴ is independently substituted or unsubstituted 4 to 5 membered heteroalkylene. In embodiments, L⁴ is independently substituted 4 to 5 membered heteroalkylene. In embodiments, L⁴ is independently unsubstituted 4 to 5 membered heteroalkylene.

R²³ is independently hydrogen or unsubstituted alkyl (e.g., C₁-C₂₃, C₁-C₁₂, C₁-C₈, C₁-C₆, C₁-C₄, or C₁-C₂). In embodiments, R²³ is independently hydrogen. In embodiments, R²³ is independently unsubstituted C₁-C₂₃ alkyl. In embodiments, R²³ is independently hydrogen or unsubstituted C₁-C₁₂ alkyl. In embodiments, R²³ is independently hydrogen or unsubstituted C₁-C₁₀ alkyl. In embodiments, R²³ is independently hydrogen or unsubstituted C₁-C₈ alkyl. In embodiments, R²³ is independently hydrogen or unsubstituted C₁-C₆ alkyl. In embodiments, R²³ is independently hydrogen or unsubstituted C₁-C₄ alkyl. In embodiments, R²³ is independently hydrogen or unsubstituted C₁-C₂ alkyl.

R²⁴ is independently hydrogen or unsubstituted alkyl (e.g., C₁-C₂₄, C₁-C₁₂, C₁-C₈, C₁-C₆, C₁-C₄, or C₁-C₂). In embodiments, R²⁴ is independently hydrogen. In embodiments, R²⁴ is independently unsubstituted C₁-C₂₄ alkyl. In embodiments, R²⁴ is independently hydrogen or unsubstituted C₁-C₁₂ alkyl. In embodiments, R²⁴ is independently hydrogen or unsubstituted C₁-C₁₀ alkyl. In embodiments, R²⁴ is independently hydrogen or unsubstituted C₁-C₈ alkyl. In embodiments, R²⁴ is independently hydrogen or unsubstituted C₁-C₆ alkyl. In embodiments, R²⁴ is independently hydrogen or unsubstituted C₁-C₄ alkyl. In embodiments, R²⁴ is independently hydrogen or unsubstituted C₁-C₂ alkyl.

R²⁵ is independently hydrogen or unsubstituted alkyl (e.g., C₁-C₂₅, C₁-C₁₂, C₁-C₈, C₁-C₆, C₁-C₄, or C₁-C₂). In embodiments, R²⁵ is independently hydrogen. In embodiments, R²⁵ is independently unsubstituted C₁-C₂₅ alkyl. In embodiments, R²⁵ is independently hydrogen or unsubstituted C₁-C₁₂ alkyl. In embodiments, R²⁵ is independently hydrogen or unsubstituted C₁-C₁₀ alkyl. In embodiments, R²⁵ is independently hydrogen or unsubstituted C₁-C₈ alkyl. In embodiments, R²⁵ is independently hydrogen or unsubstituted C₁-C₆ alkyl. In embodiments, R²⁵ is independently hydrogen or unsubstituted C₁-C₄ alkyl. In embodiments, R²⁵ is independently hydrogen or unsubstituted C₁-C₂ alkyl.

In embodiments, L³ and L⁴ are independently a bond, —NH—, —O—, —C(O)—, —C(O)O—, —OC(O)—, —OPO₂—O—, —O—P(O)(S)—O—, —O—P(O)(CH₃)—O—, —O—P(S)(CH₃)—O—, —O—P(O)(N(CH₃)₂)—N—, —O—P(O)(N(CH₃)₂)—O—, —O—P(S)(N(CH₃)₂)—N—, —O—P(S)(N(CH₃)₂)—O—, —P(O)(N(CH₃)₂)—N—, —P(O)(N(CH₃)₂)—O—, —P(S)(N(CH₃)₂)—N—, —P(S)(N(CH₃)₂)—O—, substituted or unsubstituted alkylene or substituted or unsubstituted heteroalkylene. In embodiments, L³ is independently a bond, —NH—, —O—, —C(O)—, —C(O)O—, —OC(O)—, —OPO₂—O—, —O—P(O)(S)—O—, —O—P(O)(CH₃)—O—, —O—P(S)(CH₃)—O—, —O—P(O)(N(CH₃)₂)—N—, —O—P(O)(N(CH₃)₂)—O—, —O—P(S)(N(CH₃)₂)—N—, —O—P(S)(N(CH₃)₂)—O—, —P(O)(N(CH₃)₂)—N—, —P(O)(N(CH₃)₂)—O—, —P(S)(N(CH₃)₂)—N—, —P(S)(N(CH₃)₂)—O—, substituted or unsubstituted alkylene or substituted or unsubstituted heteroalkylene. In embodiments, L⁴ is independently a bond, —NH—, —O—, —C(O)—, —C(O)O—, —OC(O)—, —OPO₂—O—, —O—P(O)(S)—O—, —O—P(O)(CH₃)—O—, —O—P(S)(CH₃)—O—, —O—P(O)(N(CH₃)₂)—N—, —O—P(O)(N(CH₃)₂)—O—, —O—P(S)(N(CH₃)₂)—N—, —O—P(S)(N(CH₃)₂)—O—, —P(O)(N(CH₃)₂)—N—, —P(O)(N(CH₃)₂)—O—, —P(S)(N(CH₃)₂)—N—, —P(S)(N(CH₃)₂)—O—, substituted or unsubstituted alkylene or substituted or unsubstituted heteroalkylene.

In embodiments, L³ is independently

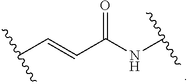 [SMILES: */C=C/C(=O)N*]

In embodiments, L³ is independently —OPO₂—O—. In embodiments, L³ is independently —O—P(O)(S)—O—. In embodiments, L³ is independently —O—. In embodiments, L³ is independently —S—.

In embodiments, L⁴ is independently substituted or unsubstituted alkylene or substituted or unsubstituted heteroalkylene. In embodiments, L⁴ is independently -L⁷-NH—C(O)— or -L⁷-C(O)—NH—. In embodiments, L⁷ is independently substituted or unsubstituted alkylene (e.g., C₁-C₂₀, C₁-C₁₂, C₁-C₈, C₁-C₆, C₁-C₄, or C₁-C₂). In embodiments, L⁷ is independently substituted alkylene (e.g., C₁-C₂₀, C₁-C₁₂, C₁-C₈, C₁-C₆, C₁-C₄, or C₁-C₂). In embodiments, L⁷ is independently unsubstituted alkylene (e.g., C₁-C₂₀, C₁-C₁₂, C₁-C₈, C₁-C₆, C₁-C₄, or C₁-C₂).

In embodiments, L⁴ is independently substituted or unsubstituted heteroalkylene (e.g., 2 to 20 membered, 2 to 12 membered, 2 to 10 membered, 2 to 8 membered, 2 to 6 membered, or 2 to 4 membered). In embodiments, L⁴ is independently substituted heteroalkylene (e.g., 2 to 20 membered, 2 to 12 membered, 2 to 10 membered, 2 to 8 membered, 2 to 6 membered, or 2 to 4 membered). In embodiments, L⁴ is independently oxo-substituted heteroalkylene (e.g., 2 to 20 membered, 2 to 12 membered, 2 to 10 membered, 2 to 8 membered, 2 to 6 membered, or 2 to 4 membered). In embodiments, L⁴ is independently unsubstituted heteroalkylene (e.g., 2 to 20 membered, 2 to 12 membered, 2 to 10 membered, 2 to 8 membered, 2 to 6 membered, or 2 to 4 membered). In embodiments, L⁴ is independently -L⁷-NH—C(O)— or -L⁷-C(O)—NH—; and L⁷ is independently substituted or unsubstituted alkylene (e.g., C₁-C₂₀, C₁-C₁₂, C₁-C₈, C₁-C₆, C₁-C₄, or C₁-C₂). In embodiments, L⁴ is independently -L⁷-NH—C(O)—; and L⁷ is independently substituted or unsubstituted alkylene (e.g., C₁-C₂₀, C₁-C₁₂, C₁-C₈, C₁-C₆, C₁-C₄, or C₁-C₂). In embodiments, L⁴ is independently -L⁷-C(O)—NH—; and L⁷ is independently substituted or unsubstituted alkylene (e.g., C₁-C₂₀, C₁-C₁₂, C₁-C₈, C₁-C₆, C₁-C₄, or C₁-C₂).

In embodiments, L⁷ is independently substituted or unsubstituted alkylene (e.g., C₁-C₂₀, C₁-C₁₂, C₁-C₈, C₁-C₆, C₁-C₄, or C₁-C₂). In embodiments, L⁷ is independently substituted alkylene (e.g., C₁-C₂₀, C₁-C₁₂, C₁-C₈, C₁-C₆, C₁-C₄, or C₁-C₂). In embodiments, L⁷ is independently unsubstituted alkylene (e.g., C₁-C₂₀, C₁-C₁₂, C₁-C₈, C₁-C₆, C₁-C₄, or C₁-C₂). In embodiments, L⁷ is independently substituted or unsubstituted C₁-C₂₀ alkylene. In embodiments, L⁷ is independently substituted C₁-C₂₀ alkylene. In embodiments, L⁷ is independently hydroxy(OH)-substituted C₁-C₂₀ alkylene. In embodiments, L⁷ is independently hydroxymethyl-substituted C₁-C₂₀ alkylene. In embodiments, L⁷ is independently unsubstituted C₁-C₂₀ alkylene. In embodiments, L⁷ is independently substituted or unsubstituted C₁-C₁₂ alkylene. In embodiments, L⁷ is independently substituted C₁-C₁₂ alkylene. In embodiments, L⁷ is independently hydroxy(OH)-substituted C₁-C₁₂ alkylene. In embodiments, L⁷ is independently hydroxymethyl-substituted C₁-C₁₂ alkylene. In embodiments, L⁷ is independently unsubstituted C₁-C₁₂ alkylene. In embodiments, L⁷ is independently substituted or unsubstituted C₁-C₈ alkylene. In embodiments, L⁷ is independently substituted C₁-C₈ alkylene. In embodiments, L⁷ is independently hydroxy(OH)-substituted C₁-C₈ alkylene. In embodiments, L⁷ is independently hydroxymethyl-substituted C₁-C₈ alkylene. In embodiments, L⁷ is independently unsubstituted C₁-C₈ alkylene. In embodiments, L⁷ is independently substituted or unsubstituted C₁-C₆ alkylene. In embodiments, L⁷ is independently substituted C₁-C₆ alkylene. In embodiments, L⁷ is independently hydroxy(OH)-substituted C₁-C₆ alkylene. In embodiments, L⁷ is independently hydroxymethyl-substituted C₁-C₆ alkylene. In embodiments, L⁷ is independently unsubstituted C₁-C₆ alkylene. In embodiments, L⁷ is independently substituted or unsubstituted C₁-C₄ alkylene. In embodiments, L⁷ is independently substituted C₁-C₄ alkylene. In embodiments, L⁷ is independently hydroxy(OH)-substituted C₁-C₄ alkylene. In embodiments, L⁷ is independently hydroxymethyl-substituted C₁-C₄ alkylene. In embodiments, L⁷ is independently unsubstituted C₁-C₄ alkylene. In embodiments, L⁷ is independently substituted or unsubstituted C₁-C₂ alkylene. In embodiments, L⁷ is independently substituted C₁-C₂ alkylene. In embodiments, L⁷ is independently hydroxy(OH)-substituted C₁-C₂ alkylene. In embodiments, L⁷ is independently hydroxymethyl-substituted C₁-C₂ alkylene. In embodiments, L⁷ is independently unsubstituted C₁-C₂ alkylene.

In embodiments, L⁴ is independently -L⁷-NH—C(O)— or -L⁷-C(O)—NH—; and L⁷ is independently substituted or unsubstituted alkylene (e.g., C₁-C₂₀, C₁-C₁₂, C₁-C₈, C₁-C₆, C₁-C₄, or C₁-C₂). In embodiments, L⁴ is independently -L⁷-NH—C(O)— or -L⁷-C(O)—NH—; and L⁷ is independently substituted or unsubstituted C₁-C₈ alkylene. In embodiments, L⁴ is independently -L⁷-NH—C(O)— or -L⁷-C(O)—NH—; and L⁷ is independently substituted C₁-C₈ alkylene. In embodiments, L⁴ is independently -L⁷-NH—C(O)— or -L⁷-C(O)—NH—; and L⁷ is independently hydroxy(OH)-substituted C₁-C₈ alkylene. In embodiments, L⁴ is independently -L⁷-NH—C(O)— or -L⁷-C(O)—NH—; and L⁷ is independently hydroxymethyl-substituted C₁-C₈ alkylene. In embodiments, L⁴ is independently -L⁷-NH—C(O)— or -L⁷-C(O)—NH—; and L⁷ is independently unsubstituted C₁-C₈ alkylene.

In embodiments, L⁴ is independently -L⁷-NH—C(O)— or -L⁷-C(O)—NH—; and L⁷ is independently substituted or unsubstituted C₃-C₈ alkylene. In embodiments, L⁴ is independently -L⁷-NH—C(O)— or -L⁷-C(O)—NH—; and L⁷ is independently substituted C₃-C₈ alkylene. In embodiments, L⁴ is independently -L⁷-NH—C(O)— or -L⁷-C(O)—NH—; and L⁷ is independently hydroxy(OH)-substituted C₃-C₈ alkylene. In embodiments, L⁴ is independently -L⁷-NH—C(O)— or -L⁷-C(O)—NH—; and L⁷ is independently hydroxymethyl-substituted C₃-C₈ alkylene. In embodiments, L⁴ is independently -L⁷-NH—C(O)— or -L⁷-C(O)—NH—; and L⁷ is independently unsubstituted C₃-C₈ alkylene.

In embodiments, L⁴ is independently -L⁷-NH—C(O)— or -L⁷-C(O)—NH—; and L⁷ is independently substituted or unsubstituted C₅-C₈ alkylene. In embodiments, L⁴ is independently -L⁷-NH—C(O)— or -L⁷-C(O)—NH—; and L⁷ is independently substituted C₅-C₈ alkylene. In embodiments, L⁴ is independently -L⁷-NH—C(O)— or -L⁷-C(O)—NH—; and L⁷ is independently hydroxy(OH)-substituted C₅-C₈ alkylene. In embodiments, L⁴ is independently -L⁷-NH—C(O)— or -L⁷-C(O)—NH—; and L⁷ is independently hydroxymethyl-substituted C₅-C₈ alkylene. In embodiments, L⁴ is independently -L⁷-NH—C(O)— or -L⁷-C(O)—NH—; and L⁷ is independently unsubstituted C₅-C₈ alkylene.

In embodiments, L⁴ is independently -L⁷-NH—C(O)— or -L⁷-C(O)—NH—; and L⁷ is independently substituted or unsubstituted octylene. In embodiments, L⁴ is independently -L⁷-NH—C(O)— or -L⁷-C(O)—NH—; and L⁷ is independently substituted octylene. In embodiments, L⁴ is independently -L⁷-NH—C(O)— or -L⁷-C(O)—NH—; and L⁷ is independently hydroxy(OH)-substituted octylene. In embodiments, L⁴ is independently -L⁷-NH—C(O)— or -L⁷-C(O)—NH—; and L⁷ is independently unsubstituted octylene. In embodiments, L⁴ is independently -L⁷-NH—C(O)— and L⁷ is independently hydroxy(OH)-substituted octylene. In embodiments, L⁴ is independently -L⁷-NH—C(O)— and L⁷ is independently hydroxymethyl-substituted octylene. In embodiments, L⁴ is independently -L⁷-NH—C(O)— and L⁷ is independently unsubstituted octylene.

In embodiments, L⁴ is independently -L⁷-NH—C(O)— or -L⁷-C(O)—NH—; and L⁷ is independently substituted or unsubstituted heptylene. In embodiments, L⁴ is independently -L⁷-NH—C(O)— or -L⁷-C(O)—NH—; and L⁷ is independently substituted heptylene. In embodiments, L⁴ is independently -L⁷-NH—C(O)— or -L⁷-C(O)—NH—; and L⁷ is independently hydroxy(OH)-substituted heptylene. In embodiments, L⁴ is independently -L⁷-NH—C(O)— or -L⁷-C(O)—NH—; and L⁷ is independently unsubstituted heptylene. In embodiments, L⁴ is independently -L⁷-NH—C(O)— and L⁷ is independently hydroxy(OH)-substituted heptylene. In embodiments, L⁴ is independently -L⁷-NH—C(O)— and L⁷ is independently hydroxymethyl-substituted heptylene. In embodiments, L⁴ is independently -L⁷-NH—C(O)— and L⁷ is independently unsubstituted heptylene.

In embodiments, L⁴ is independently -L⁷-NH—C(O)— or -L⁷-C(O)—NH—; and L⁷ is independently substituted or unsubstituted hexylene. In embodiments, L⁴ is independently -L⁷-NH—C(O)— or -L⁷-C(O)—NH—; and L⁷ is independently substituted hexylene. In embodiments, L⁴ is independently -L⁷-NH—C(O)— or -L⁷-C(O)—NH—; and L⁷ is independently hydroxy(OH)-substituted hexylene. In embodiments, L⁴ is independently -L⁷-NH—C(O)— or -L⁷-C(O)—NH—; and L⁷ is independently unsubstituted hexylene. In embodiments, L⁴ is independently -L⁷-NH—C(O)— and L⁷ is independently hydroxy(OH)-substituted hexylene. In embodiments, L⁴ is independently -L⁷-NH—C(O)— and L⁷ is independently hydroxymethyl-substituted hexylene. In embodiments, L⁴ is independently -L⁷-NH—C(O)— and L⁷ is independently unsubstituted hexylene.

In embodiments, L⁴ is independently -L⁷-NH—C(O)— or -L⁷-C(O)—NH—; and L⁷ is independently substituted or unsubstituted pentylene. In embodiments, L⁴ is independently -L⁷-NH—C(O)— or -L⁷-C(O)—NH—; and L⁷ is independently substituted pentylene. In embodiments, L⁴ is independently -L⁷-NH—C(O)— or -L⁷-C(O)—NH—; and L⁷ is independently hydroxy(OH)-substituted pentylene. In embodiments, L⁴ is independently -L⁷-NH—C(O)— or -L⁷-C(O)—NH—; and L⁷ is independently unsubstituted pentylene. In embodiments, L⁴ is independently -L⁷-NH—C(O)— and L⁷ is independently hydroxy(OH)-substituted pentylene. In embodiments, L⁴ is independently -L⁷-NH—C(O)— and L⁷ is independently hydroxymethyl-substituted pentylene. In embodiments, L⁴ is independently -L⁷-NH—C(O)— and L⁷ is independently unsubstituted pentylene.

In embodiments, L⁴ is independently

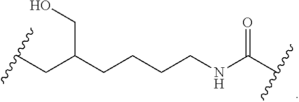 [SMILES: *CC(CO)CCCCNC(*)=O]

In embodiments, L⁴ is independently

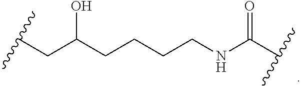 [SMILES: *CC(O)CCCCNC(*)=O]

In embodiments, L⁴ is independently

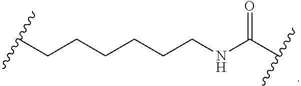 [SMILES: *CCCCCCNC(*)=O]

In embodiments, L⁴ is independently

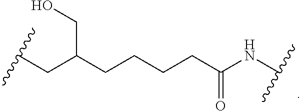 [SMILES: *CC(CO)CCCCC(=O)N*]

In embodiments, L⁴ is independently

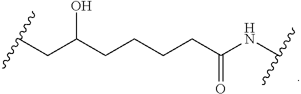 [SMILES: *CC(O)CCCCC(=O)N*]

In embodiments, L⁴ is independently

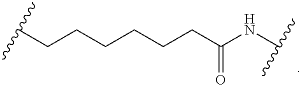 [SMILES: *CCCCCCC(=O)N*]

In embodiments, L⁴ is independently

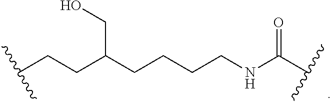 [SMILES: *CCC(CO)CCCCNC(*)=O]

In embodiments, L⁴ is

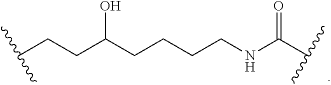 [SMILES: *CCC(O)CCCCNC(*)=O]

In embodiments L⁴ is independently

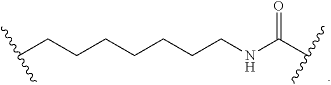 [SMILES: *CCCCCCCNC(*)=O]

In embodiments, L⁴ is independently

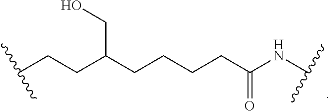 [SMILES: *CCC(CO)CCCCC(=O)N*]

In embodiments, L⁴ is independently

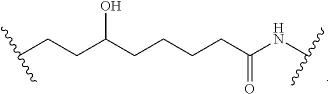 [SMILES: *CCC(O)CCCCC(=O)N*]

In embodiments, L⁴ is independently

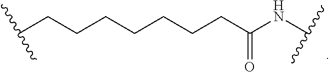 [SMILES: *CCCCCCCC(=O)N*]

In embodiments, -L³-L⁴- is independently -L⁷-NH—C(O)— or -L⁷-C(O)—NH—. In embodiments, L⁷ is independently substituted or unsubstituted heteroalkylene (e.g., 2 to 20 membered, 2 to 12 membered, 2 to 10 membered, 2 to 8 membered, 2 to 6 membered, or 2 to 4 membered). In embodiments, L⁷ is independently substituted heteroalkylene (e.g., 2 to 20 membered, 2 to 12 membered, 2 to 10 membered, 2 to 8 membered, 2 to 6 membered, or 2 to 4 membered). In embodiments, L⁷ is independently oxo-substituted heteroalkylene (e.g., 2 to 20 membered, 2 to 12 membered, 2 to 10 membered, 2 to 8 membered, 2 to 6 membered, or 2 to 4 membered). In embodiments, L⁷ is independently unsubstituted heteroalkylene (e.g., 2 to 20 membered, 2 to 12 membered, 2 to 10 membered, 2 to 8 membered, 2 to 6 membered, or 2 to 4 membered). In embodiments, L⁷ is independently substituted or unsubstituted heteroalkenylene (e.g., 2 to 20 membered, 2 to 12 membered, 2 to 10 membered, 2 to 8 membered, 2 to 6 membered, or 2 to 4 membered). In embodiments, L⁷ is independently substituted heteroalkenylene (e.g., 2 to 20 membered, 2 to 12 membered, 2 to 10 membered, 2 to 8 membered, 2 to 6 membered, or 2 to 4 membered). In embodiments, L⁷ is independently oxo-substituted heteroalkenylene (e.g., 2 to 20 membered, 2 to 12 membered, 2 to 10 membered, 2 to 8 membered, 2 to 6 membered, or 2 to 4 membered). In embodiments, L⁷ is independently unsubstituted heteroalkenylene (e.g., 2 to 20 membered, 2 to 12 membered, 2 to 10 membered, 2 to 8 membered, 2 to 6 membered, or 2 to 4 membered).

In embodiments, L⁷ is independently substituted or unsubstituted 2 to 20 membered heteroalkylene. In embodiments, L⁷ is independently substituted 2 to 20 membered heteroalkylene. In embodiments, L⁷ is independently oxo-substituted 2 to 20 membered heteroalkylene. In embodiments, L⁷ is independently unsubstituted 2 to 20 membered heteroalkylene. In embodiments, L⁷ is independently substituted or unsubstituted 2 to 12 membered heteroalkylene. In embodiments, L⁷ is independently substituted 2 to 12 membered heteroalkylene. In embodiments, L⁷ is independently oxo-substituted 2 to 12 membered heteroalkylene. In embodiments, L⁷ is independently unsubstituted 2 to 12 membered heteroalkylene. In embodiments, L⁷ is independently substituted or unsubstituted 2 to 10 membered heteroalkylene. In embodiments, L⁷ is independently substituted 2 to 10 membered heteroalkylene. In embodiments, L⁷ is independently oxo-substituted 2 to 10 membered heteroalkylene. In embodiments, L⁷ is independently unsubstituted 2 to 10 membered heteroalkylene. In embodiments, L⁷ is independently substituted or unsubstituted 2 to 8 membered heteroalkylene. In embodiments, L⁷ is independently substituted 2 to 8 membered heteroalkylene. In embodiments, L⁷ is independently oxo-substituted 2 to 8 membered heteroalkylene. In embodiments, L⁷ is independently unsubstituted 2 to 8 membered heteroalkylene. In embodiments, L⁷ is independently substituted or unsubstituted 2 to 6 membered heteroalkylene. In embodiments, L⁷ is independently substituted 2 to 6 membered heteroalkylene. In embodiments, L⁷ is independently oxo-substituted 2 to 6 membered heteroalkylene. In embodiments, L⁷ is independently unsubstituted 2 to 6 membered heteroalkylene. In embodiments, L⁷ is independently substituted or unsubstituted 2 to 4 membered heteroalkylene. In embodiments, L⁷ is independently substituted 2 to 4 membered heteroalkylene. In embodiments, L⁷ is independently oxo-substituted 2 to 4 membered heteroalkylene. In embodiments, L⁷ is independently unsubstituted 2 to 4 membered heteroalkylene.

In embodiments, L⁷ is independently substituted or unsubstituted 2 to 20 membered heteroalkenylene. In embodiments, L⁷ is independently substituted 2 to 20 membered heteroalkenylene. In embodiments, L⁷ is independently oxo-substituted 2 to 20 membered heteroalkenylene. In embodiments, L⁷ is independently unsubstituted 2 to 20 membered heteroalkenylene. In embodiments, L⁷ is independently substituted or unsubstituted 2 to 12 membered heteroalkenylene. In embodiments, L⁷ is independently substituted 2 to 12 membered heteroalkenylene. In embodiments, L⁷ is independently oxo-substituted 2 to 12 membered heteroalkenylene. In embodiments, L⁷ is independently unsubstituted 2 to 12 membered heteroalkenylene. In embodiments, L⁷ is independently substituted or unsubstituted 2 to 10 membered heteroalkenylene. In embodiments, L⁷ is independently substituted 2 to 10 membered heteroalkenylene. In embodiments, L⁷ is independently oxo-substituted 2 to 10 membered heteroalkenylene. In embodiments, L⁷ is independently unsubstituted 2 to 10 membered heteroalkenylene. In embodiments, L⁷ is independently substituted or unsubstituted 2 to 8 membered heteroalkenylene. In embodiments, L⁷ is independently substituted 2 to 8 membered heteroalkenylene. In embodiments, L⁷ is independently oxo-substituted 2 to 8 membered heteroalkenylene. In embodiments, L⁷ is independently unsubstituted 2 to 8 membered heteroalkenylene. In embodiments, L⁷ is independently substituted or unsubstituted 2 to 6 membered heteroalkenylene. In embodiments, L⁷ is independently substituted 2 to 6 membered heteroalkenylene. In embodiments, L⁷ is independently oxo-substituted 2 to 6 membered heteroalkenylene. In embodiments, L⁷ is independently unsubstituted 2 to 6 membered heteroalkenylene. In embodiments, L⁷ is independently substituted or unsubstituted 2 to 4 membered heteroalkenylene. In embodiments, L⁷ is independently substituted 2 to 4 membered heteroalkenylene. In embodiments, L⁷ is independently oxo-substituted 2 to 4 membered heteroalkenylene. In embodiments, L⁷ is independently unsubstituted 2 to 4 membered heteroalkenylene.

In embodiments, -L³-L⁴- is independently —O-L⁷-NH—C(O)— or —O-L⁷-C(O)—NH—. In embodiments, L⁷ is independently substituted or unsubstituted alkylene (e.g., C₁-C₂₀, C₁-C₁₂, C₁-C₈, C₁-C₆, C₁-C₄, or C₁-C₂). In embodiments, -L³-L⁴- is independently —O-L⁷-NH—C(O)— or —O-L⁷-C(O)—NH—; and L⁷ is independently substituted or unsubstituted alkylene (e.g., C₁-C₂₀, C₁-C₁₂, C₁-C₈, C₁-C₆, C₁-C₄, or C₁-C₂). In embodiments, -L³-L⁴- is independently —O-L⁷-NH—C(O)—; and L⁷ is independently substituted or unsubstituted alkylene (e.g., C₁-C₂₀, C₁-C₁₂, C₁-C₈, C₁-C₆, C₁-C₄, or C₁-C₂). In embodiments, -L³-L⁴- is independently —O-L⁷-C(O)—NH—; and L⁷ is independently substituted or unsubstituted alkylene (e.g., C₁-C₂₀, C₁-C₁₂, C₁-C₈, C₁-C₆, C₁-C₄, or C₁-C₂).

In embodiments, L³-L⁴- is independently —O-L⁷-C(O)—NH—; and L⁷ is independently substituted or unsubstituted C₁-C₈ alkylene. In embodiments, -L³-L⁴- is independently —O-L⁷-C(O)—NH—; and L⁷ is independently substituted C₁-C₈ alkylene. In embodiments, -L³-L⁴- is independently —O-L⁷-C(O)—NH—; and L⁷ is independently hydroxy(OH)-substituted C₁-C₈ alkylene. In embodiments, -L³-L⁴- is independently —O-L⁷-C(O)—NH— and L⁷ is independently hydroxymethyl-substituted C₁-C₈ alkylene. In embodiments, -L³-L⁴- is independently —O-L⁷-C(O)—NH—; and L⁷ is independently unsubstituted C₁-C₈ alkylene.

In embodiments, -L³-L⁴- is independently —O-L⁷-C(O)—NH—; and L⁷ is independently substituted or unsubstituted C₃-C₈ alkylene. In embodiments, -L³-L⁴- is independently O-L⁷-C(O)—NH—; and L⁷ is independently substituted C₃-C₈ alkylene. In embodiments, -L³-L⁴- is independently —O-L⁷-C(O)—NH—; and L⁷ is independently hydroxy(OH)-substituted C₃-C₈ alkylene. In embodiments, -L³-L⁴- is independently —O-L⁷-C(O)—NH— and L⁷ is independently hydroxymethyl-substituted C₃-C₈ alkylene. In embodiments, -L³-L⁴- is independently —O-L⁷-C(O)—NH—; and L⁷ is independently unsubstituted C₃-C₈ alkylene.

In embodiments, -L³-L⁴- is independently —O-L⁷-C(O)—NH—; and L⁷ is independently substituted or unsubstituted C₅-C₈ alkylene. In embodiments, -L³-L⁴- is independently —O-L⁷-C(O)—NH—; and L⁷ is independently substituted C₅-C₈ alkylene. In embodiments, -L³-L⁴- is independently —O-L⁷-C(O)—NH—; and L⁷ is independently hydroxy(OH)-substituted C₅-C₈ alkylene. In embodiments, -L³-L⁴- is independently —O-L⁷-C(O)—NH— and L⁷ is independently hydroxymethyl-substituted C₅-C₈ alkylene. In embodiments, -L³-L⁴- is independently —O-L⁷-C(O)—NH—; and L⁷ is independently unsubstituted C₅-C₈ alkylene.

In embodiments, -L³-L⁴- is independently —O-L⁷-NH—C(O)—; and L⁷ is independently substituted or unsubstituted C₁-C₈ alkylene. In embodiments, -L³-L⁴- is independently —O-L⁷-NH—C(O)—; and L⁷ is independently substituted C₁-C₈ alkylene. In embodiments, -L³-L⁴- is independently —O-L⁷-NH—C(O)—; and L⁷ is independently hydroxy(OH)-substituted C₁-C₈ alkylene. In embodiments, -L³-L⁴- is independently —O-L⁷-NH—C(O)—; and L⁷ is independently hydroxymethyl-substituted C₁-C₈ alkylene. In embodiments, -L³-L⁴- is independently —O-L⁷-NH—C(O)—; and L⁷ is independently unsubstituted C₁-C₈ alkylene.

In embodiments, -L³-L⁴- is independently —O-L⁷-NH—C(O)—; and L⁷ is independently substituted or unsubstituted C₃-C₈ alkylene. In embodiments, -L³-L⁴- is independently —O-L⁷-NH—C(O)—; and L⁷ is independently substituted C₃-C₈ alkylene. In embodiments, -L³-L⁴- is independently —O-L⁷-NH—C(O)—; and L⁷ is independently hydroxy(OH)-substituted C₃-C₈ alkylene. In embodiments, -L³-L⁴- is independently —O-L⁷-NH—C(O)—; and L⁷ is independently hydroxymethyl-substituted C₃-C₈ alkylene. In embodiments, -L³-L⁴- is independently —O-L⁷-NH—C(O)—; and L⁷ is independently unsubstituted C₃-C₈ alkylene.

In embodiments, -L³-L⁴- is independently —O-L⁷-NH—C(O)—; and L⁷ is independently substituted or unsubstituted C₅-C₈ alkylene. In embodiments, -L³-L⁴- is independently —O-L⁷-NH—C(O)—; and L⁷ is independently substituted C₅-C₈ alkylene. In embodiments, -L³-L⁴- is independently —O-L⁷-NH—C(O)—; and L⁷ is independently hydroxy(OH)-substituted C₅-C₈ alkylene. In embodiments, -L³-L⁴- is independently —O-L⁷-NH—C(O)—; and L⁷ is independently hydroxymethyl-substituted C₅-C₈ alkylene. In embodiments, -L³-L⁴- is independently —O-L⁷-NH—C(O)—; and L⁷ is independently unsubstituted C₅-C₈ alkylene.

In embodiments, -L³-L⁴- is independently

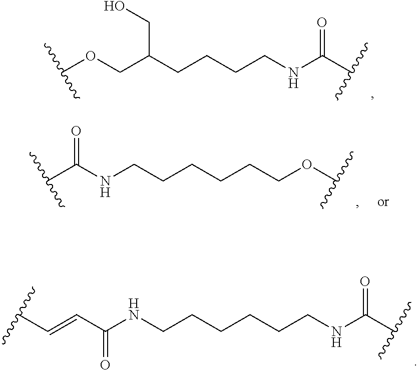 [SMILES: *OCC(CO)CCCCNC(*)=O ; *OCCCCCCNC(*)=O ; */C=C/C(=O)NCCCCCCNC(*)=O]

In embodiments, -L³-L⁴- is independently

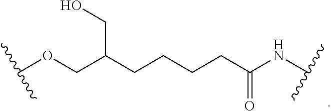 [SMILES: *NC(=O)CCCCC(CO)CO*]

In embodiments, -L³-L⁴- is independently

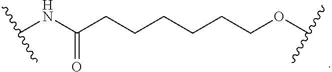 [SMILES: *NC(=O)CCCCCCO*]

In embodiments, -L³-L⁴- is independently

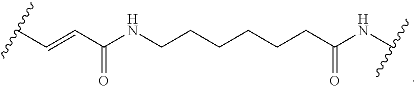 [SMILES: */C=C/C(=O)NCCCCCCC(=O)N*]

In embodiments, -L³-L⁴- is independently —OPO₂—O-L⁷-NH—C(O)—, —OP(O)(S)—O-L⁷-NH—C(O)—, —OPO₂—O-L⁷-C(O)—NH— or —OP(O)(S)—O-L⁷-C(O)—NH—. In embodiments, L⁷ is independently substituted or unsubstituted alkylene (e.g., C₁-C₂₀, C₁-C₁₂, C₁-C₈, C₁-C₆, C₁-C₄, or C₁-C₂). In embodiments, -L³-L⁴- is independently —OPO₂—O-L⁷-NH—C(O)— or —OP(O)(S)—O-L⁷-NH—C(O)—; and L⁷ is independently substituted or unsubstituted alkylene. In embodiments, -L³-L⁴- is independently —OPO₂—O-L⁷-NH—C(O)—; and L⁷ is independently substituted or unsubstituted alkylene. In embodiments, -L³-L⁴- is independently —OP(O)(S)—O-L⁷-NH—C(O)—; and L⁷ is independently substituted or unsubstituted alkylene. In embodiments, -L³-L⁴- is independently —OPO₂—O-L⁷-C(O)—NH— or —OP(O)(S)—O-L⁷-C(O)—NH—; and L⁷ is independently substituted or unsubstituted alkylene. In embodiments, -L³-L⁴- is independently —OPO₂—O-L⁷-C(O)—NH—; and L⁷ is independently substituted or unsubstituted alkylene. In embodiments, -L³-L⁴- is independently —OP(O)(S)—O-L⁷-C(O)—NH—; and L⁷ is independently substituted or unsubstituted alkylene.

In embodiments, -L³-L⁴- is independently —OPO₂—O-L⁷-NH—C(O)— or —OPO₂—O-L⁷-C(O)—NH—; and L⁷ is independently substituted or unsubstituted alkylene (e.g., C₁-C₂₀, C₁-C₁₂, C₁-C₈, C₁-C₆, C₁-C₄, or C₁-C₂). In embodiments, -L³-L⁴- is independently —OPO₂—O-L⁷-NH—C(O)—; and L⁷ is independently substituted or unsubstituted alkylene (e.g., C₁-C₂₀, C₁-C₁₂, C₁-C₈, C₁-C₆, C₁-C₄, or C₁-C₂). In embodiments, -L³-L⁴- is independently —OPO₂—O-L⁷-C(O)—NH—; and L⁷ is independently substituted or unsubstituted alkylene (e.g., C₁-C₂₀, C₁-C₁₂, C₁-C₈, C₁-C₆, C₁-C₄, or C₁-C₂).

In embodiments, -L³-L⁴- is independently —OP(O)(S)—O-L⁷-NH—C(O)— or —OP(O)(S)—O-L⁷-C(O)—NH—; and L⁷ is independently substituted or unsubstituted alkylene (e.g., C₁-C₂₀, C₁-C₁₂, C₁-C₈, C₁-C₆, C₁-C₄, or C₁-C₂). In embodiments, -L³-L⁴- is independently —OP(O)(S)—O-L⁷-NH—C(O)—; and L⁷ is independently substituted or unsubstituted alkylene (e.g., C₁-C₂₀, C₁-C₁₂, C₁-C₈, C₁-C₆, C₁-C₄, or C₁-C₂). In embodiments, -L³-L⁴- is independently —OP(O)(S)—O-L⁷-C(O)—NH—; and L⁷ is independently substituted or unsubstituted alkylene (e.g., C₁-C₂₀, C₁-C₁₂, C₁-C₈, C₁-C₆, C₁-C₄, or C₁-C₂). In embodiments, -L³-L⁴- is independently —OPO₂—O-L⁷-C(O)—NH—; and L⁷ is independently substituted or unsubstituted C₁-C₈ alkylene. In embodiments, -L³-L⁴- is independently —OPO₂—O-L⁷-C(O)—NH—; and L⁷ is independently substituted C₁-C₈ alkylene. In embodiments, -L³-L⁴- is independently —OPO₂—O-L⁷-C(O)—NH—; and L⁷ is independently hydroxy(OH)-substituted C₁-C₈ alkylene. In embodiments, -L³-L⁴- is independently —OPO₂—O-L⁷-C(O)—NH—; and L⁷ is independently hydroxymethyl-substituted C₁-C₈ alkylene. In embodiments, -L³-L⁴- is independently —OPO₂—O-L⁷-C(O)—NH—; and L⁷ is independently unsubstituted C₁-C₈ alkylene.

In embodiments, -L³-L⁴- is independently —OP(O)(S)—O-L⁷-C(O)—NH—; and L⁷ is independently substituted or unsubstituted C₁-C₈ alkylene. In embodiments, -L³-L⁴- is independently —OP(O)(S)—O-L⁷-C(O)—NH—; and L⁷ is independently substituted C₁-C₈ alkylene. In embodiments, -L³-L⁴- is independently —OP(O)(S)—O-L⁷-C(O)—NH—; and L⁷ is independently hydroxy(OH)-substituted C₁-C₈ alkylene. In embodiments, -L³-L⁴- is independently —OP(O)(S)—O-L⁷-C(O)—NH—; and L⁷ is independently hydroxymethyl-substituted C₁-C₈ alkylene. In embodiments, -L³-L⁴- is independently —OP(O)(S)—O-L⁷-C(O)—NH—; and L⁷ is independently unsubstituted C₁-C₈ alkylene.

In embodiments, -L³-L⁴- is independently —OPO₂—O-L⁷-C(O)—NH—; and L⁷ is independently substituted or unsubstituted C₃-C₈ alkylene. In embodiments, -L³-L⁴- is independently —OPO₂—O-L⁷-C(O)—NH—; and L⁷ is independently substituted C₃-C₈ alkylene. In embodiments, -L³-L⁴- is independently —OPO₂—O-L⁷-C(O)—NH—; and L⁷ is independently hydroxy(OH)-substituted C₃-C₈ alkylene. In embodiments, -L³-L⁴- is independently —OPO₂—O-L⁷-C(O)—NH—; and L⁷ is independently hydroxymethyl-substituted C₃-C₈ alkylene. In embodiments, -L³-L⁴- is independently —OPO₂—O-L⁷-C(O)—NH—; and L⁷ is independently unsubstituted C₃-C₈ alkylene.

In embodiments, -L³-L⁴- is independently —OP(O)(S)—O-L⁷-C(O)—NH—; and L⁷ is independently substituted or unsubstituted C₃-C₈ alkylene. In embodiments, -L³-L⁴- is independently —OP(O)(S)—O-L⁷-C(O)—NH—; and L⁷ is independently substituted C₃-C₈ alkylene. In embodiments, -L³-L⁴- is independently —OP(O)(S)—O-L⁷-C(O)—NH—; and L⁷ is independently hydroxy(OH)-substituted C₃-C₈ alkylene. In embodiments, -L³-L⁴- is independently —OP(O)(S)—O-L⁷-C(O)—NH—; and L⁷ is independently hydroxymethyl-substituted C₃-C₈ alkylene. In embodiments, -L³-L⁴- is independently —OP(O)(S)—O-L⁷-C(O)—NH—; and L⁷ is independently unsubstituted C₃-C₈ alkylene.

In embodiments, -L³-L⁴- is independently —OPO₂—O-L⁷-C(O)—NH—; and L⁷ is independently substituted or unsubstituted C₅-C₈ alkylene. In embodiments, -L³-L⁴- is independently —OPO₂—O-L⁷-C(O)—NH—; and L⁷ is independently substituted C₅-C₈ alkylene. In embodiments, -L³-L⁴- is independently —OPO₂—O-L⁷-C(O)—NH—; and L⁷ is independently hydroxy(OH)-substituted C₅-C₈ alkylene. In embodiments, -L³-L⁴- is independently —OPO₂—O-L⁷-C(O)—NH—; and L⁷ is independently hydroxymethyl-substituted C₅-C₈ alkylene. In embodiments, -L³-L⁴- is independently —OPO₂—O-L⁷-C(O)—NH—; and L⁷ is independently unsubstituted C₅-C₈ alkylene.

In embodiments, -L³-L⁴- is independently —OP(O)(S)—O-L⁷-C(O)—NH—; and L⁷ is independently substituted or unsubstituted C₅-C₈ alkylene. In embodiments, -L³-L⁴- is independently —OP(O)(S)—O-L⁷-C(O)—NH—; and L⁷ is independently substituted C₅-C₈ alkylene. In embodiments, -L³-L⁴- is independently —OP(O)(S)—O-L⁷-C(O)—NH—; and L⁷ is independently hydroxy(OH)-substituted C₅-C₈ alkylene. In embodiments, -L³-L⁴- is independently —OP(O)(S)—O-L⁷-C(O)—NH—; and L⁷ is independently hydroxymethyl-substituted C₅-C₈ alkylene. In embodiments, -L³-L⁴- is independently —OP(O)(S)—O-L⁷-C(O)—NH—; and L⁷ is independently unsubstituted C₅-C₈ alkylene.

In embodiments, -L³-L⁴- is independently —OPO₂—O-L⁷-NH—C(O)—; and L⁷ is independently substituted or unsubstituted C₁-C₈ alkylene. In embodiments, -L³-L⁴- is independently —OPO₂—O-L⁷-NH—C(O)—; and L⁷ is independently substituted C₁-C₈ alkylene. In embodiments, -L³-L⁴- is independently —OPO₂—O-L⁷-NH—C(O)—; and L⁷ is independently hydroxy(OH)-substituted C₁-C₈ alkylene. In embodiments, -L³-L⁴- is independently —OPO₂—O-L⁷-NH—C(O)—; and L⁷ is independently hydroxymethyl-substituted C₁-C₈ alkylene. In embodiments, -L³-L⁴- is independently —OPO₂—O-L⁷-NH—C(O)—; and L⁷ is independently unsubstituted C₁-C₈ alkylene.

In embodiments, -L³-L⁴- is independently —OP(O)(S)—O-L⁷-NH—C(O)—; and L⁷ is independently substituted or unsubstituted C₁-C₈ alkylene. In embodiments, -L³-L⁴- is independently —OP(O)(S)—O-L⁷-NH—C(O)—; and L⁷ is independently substituted C₁-C₈ alkylene. In embodiments, -L³-L⁴- is independently —OP(O)(S)₂—O-L⁷-NH—C(O)—; and L⁷ is independently hydroxy(OH)-substituted C₁-C₈ alkylene. In embodiments, -L³-L⁴- is independently —OP(O)(S)—O-L⁷-NH—C(O)—; and L⁷ is independently hydroxymethyl-substituted C₁-C₈ alkylene. In embodiments, -L³-L⁴- is independently —OP(O)(S)—O-L⁷-NH—C(O)—; and L⁷ is independently unsubstituted C₁-C₈ alkylene.

In embodiments, -L³-L⁴- is independently —OPO₂—O-L⁷-NH—C(O)—; and L⁷ is independently substituted or unsubstituted C₃-C₈ alkylene. In embodiments, -L³-L⁴- is independently —OPO₂—O-L⁷-NH—C(O)—; and L⁷ is independently substituted C₃-C₈ alkylene. In embodiments, -L³-L⁴- is independently —OPO₂—O-L⁷-NH—C(O)—; and L⁷ is independently hydroxy(OH)-substituted C₃-C₈ alkylene. In embodiments, -L³-L⁴- is independently —OPO₂—O-L⁷-NH—C(O)—; and L⁷ is independently hydroxymethyl-substituted C₃-C₈ alkylene. In embodiments, -L³-L⁴- is independently —OPO₂—O-L⁷-NH—C(O)—; and L⁷ is independently unsubstituted C₃-C₈ alkylene.

In embodiments, -L³-L⁴- is independently —OP(O)(S)—O-L⁷-NH—C(O)—; and L⁷ is independently substituted or unsubstituted C₃-C₈ alkylene. In embodiments, -L³-L⁴- is independently —OP(O)(S)—O-L⁷-NH—C(O)—; and L⁷ is independently substituted C₃-C₈ alkylene. In embodiments, -L³-L⁴- is independently —OP(O)(S)—O-L⁷-NH—C(O)—; and L⁷ is independently hydroxy(OH)-substituted C₃-C₈ alkylene. In embodiments, -L³-L⁴- is independently —OP(O)(S)—O-L⁷-NH—C(O)—; and L⁷ is independently hydroxymethyl-substituted C₃-C₈ alkylene. In embodiments, -L³-L⁴- is independently —OP(O)(S)—O-L⁷-NH—C(O)—; and L⁷ is independently unsubstituted C₃-C₈ alkylene.

In embodiments, -L³-L⁴- is independently —OPO₂—O-L⁷-NH—C(O)—; and L⁷ is independently substituted or unsubstituted C₅-C₈ alkylene. In embodiments, -L³-L⁴- is independently —OPO₂—O-L⁷-NH—C(O)—; and L⁷ is independently substituted C₅-C₈ alkylene. In embodiments, -L³-L⁴- is independently —OPO₂—O-L⁷-NH—C(O)—; and L⁷ is independently hydroxy(OH)-substituted C₅-C₈ alkylene. In embodiments, -L³-L⁴- is independently —OPO₂—O-L⁷-NH—C(O)—; and L⁷ is independently hydroxymethyl-substituted C₅-C₈ alkylene. In embodiments, -L³-L⁴- is independently —OPO₂—O-L⁷-NH—C(O)—; and L⁷ is independently unsubstituted C₅-C₈ alkylene.

In embodiments, -L³-L⁴- is independently —OP(O)(S)—O-L⁷-NH—C(O)—; and L⁷ is independently substituted or unsubstituted C₅-C₈ alkylene. In embodiments, -L³-L⁴- is independently —OP(O)(S)—O-L⁷-NH—C(O)—; and L⁷ is independently substituted C₅-C₈ alkylene. In embodiments, -L³-L⁴- is independently —OP(O)(S)—O-L⁷-NH—C(O)—; and L⁷ is independently hydroxy(OH)-substituted C₅-C₈ alkylene. In embodiments, -L³-L⁴- is independently —OP(O)(S)—O-L⁷-NH—C(O)—; and L⁷ is independently hydroxymethyl-substituted C₅-C₈ alkylene. In embodiments, -L³-L⁴- is independently —OP(O)(S)—O-L⁷-NH—C(O)—; and L⁷ is independently unsubstituted C₅-C₈ alkylene.

In embodiments, -L³-L⁴- is attached to a 3′ carbon of a nucleotide of the sense strand. In embodiments, -L³-L⁴- is attached to the 3′ carbon of the 3′ terminal nucleotide of the sense strand. In embodiments, -L³-L⁴- is attached to a 3′ carbon of the antisense sense strand. In embodiments, -L³-L⁴- is attached to the 3′ carbon of the 3′ terminal nucleotide of the antisense sense strand.

In embodiments, -L³-L⁴- is attached to a 5′ carbon of a nucleotide of the sense strand. In embodiments, -L³-L⁴- is attached to the 5′ carbon of the 5′ terminal nucleotide of the sense strand. In embodiments, -L³-L⁴- is attached to a 5′ carbon of a nucleotide of the antisense strand. In embodiments, -L³-L⁴- is attached to the 5′ carbon of the 5′ terminal nucleotide of the antisense strand.

In embodiments, -L³-L⁴- is attached to a 2′ carbon of a nucleotide of the sense strand. In embodiments, -L³-L⁴- is attached to a 2′ carbon of a nucleotide of the antisense strand.

In embodiments, -L³-L⁴- is attached to a nucleobase of the sense strand. In embodiments, -L³-L⁴- is attached to a nucleobase of the antisense strand.

In embodiments, -L³-L⁴- is independently

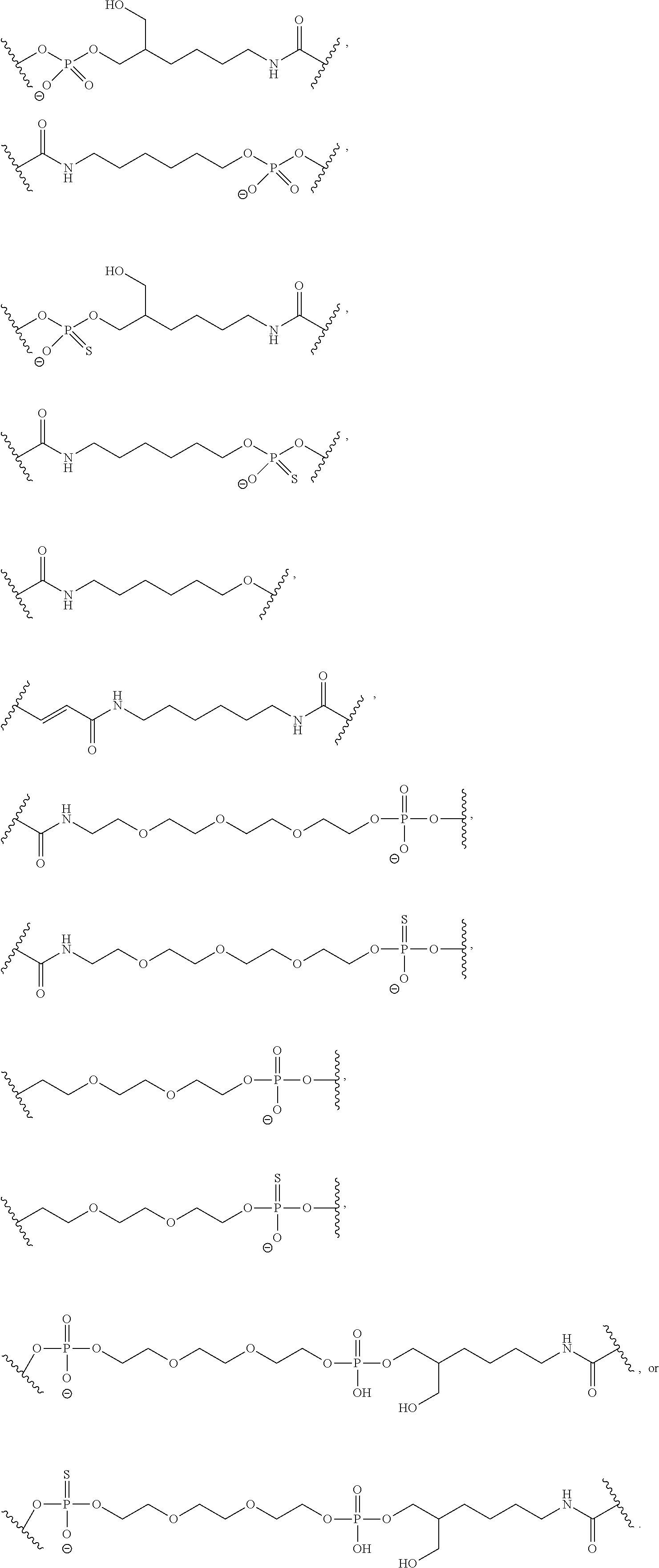

In embodiments, -L³-L⁴- is independently

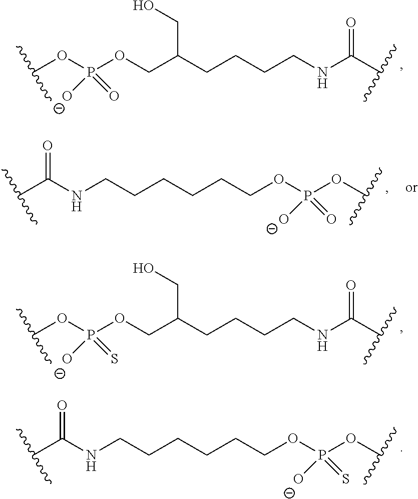 [SMILES: *OP(=O)([O-])OCC(CO)CCCCNC(*)=O ; *OP(=O)([O-])OCCCCCCNC(*)=O ; *OP([O-])(=S)OCC(CO)CCCCNC(*)=O ; *OP([O-])(=S)OCCCCCCNC(*)=O]

In embodiments, -L³-L⁴- is independently

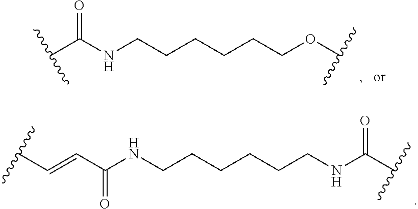 [SMILES: *OCCCCCCNC(*)=O ; */C=C/C(=O)NCCCCCCNC(*)=O]

In embodiments, -L³-L⁴- is independently

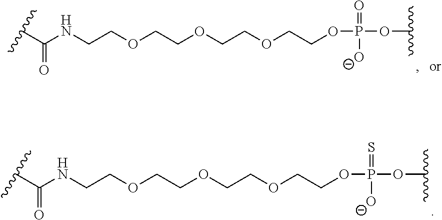 [SMILES: *OP(=O)([O-])OCCOCCOCCOCCNC(*)=O ; *OP([O-])(=S)OCCOCCOCCOCCNC(*)=O]

In embodiments, -L³-L⁴- is independently

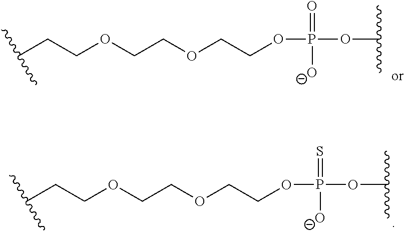 [SMILES: *CCOCCOCCOP(=O)([O-])O* ; *CCOCCOCCOP([O-])(=S)O*]

In embodiments, -L³-L⁴- is independently

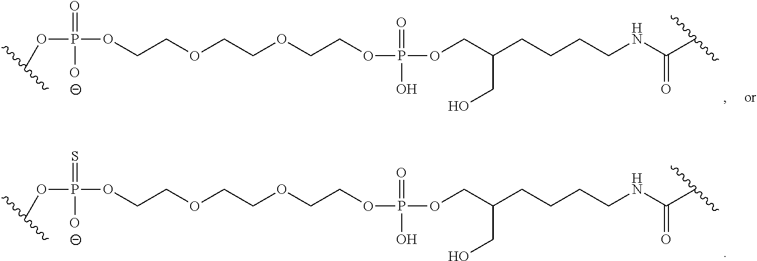 [SMILES: *OP(=O)([O-])OCCOCCOCCOP(=O)(O)OCC(CO)CCCCNC(*)=O ; *OP([O-])(=S)OCCOCCOCCOP(=O)(O)OCC(CO)CCCCNC(*)=O]

In embodiments, -L³-L⁴- is independently

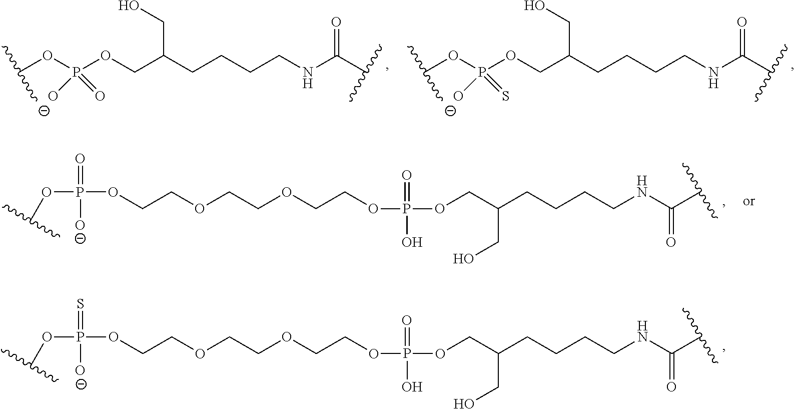 [SMILES: *OP(=O)([O-])OCC(CO)CCCCNC(*)=O ; *OP([O-])(=S)OCC(CO)CCCCNC(*)=O ; *OP(=O)([O-])OCCOCCOCCOP(=O)(O)OCC(CO)CCCCNC(*)=O ; *OP([O-])(=S)OCCOCCOCCOP(=O)(O)OCC(CO)CCCCNC(*)=O]

and is attached to the 3′ carbon of the 3′ terminal nucleotide of the sense strand.

In embodiments, -L³-L⁴- is independently

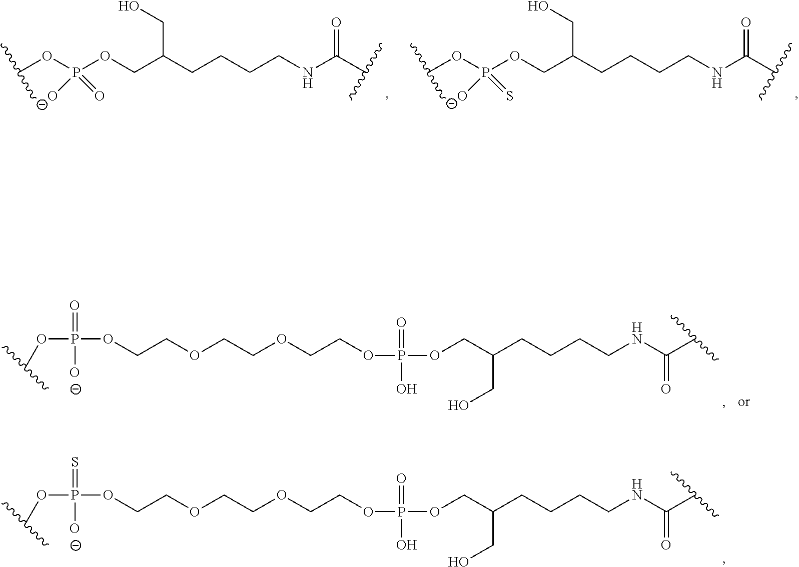 [SMILES: *OP(=O)([O-])OCC(CO)CCCCNC(*)=O ; *OP([O-])(=S)OCC(CO)CCCCNC(*)=O ; *OP(=O)([O-])OCCOCCOCCOP(=O)(O)OCC(CO)CCCCNC(*)=O ; *OP([O-])(=S)OCCOCCOCCOP(=O)(O)OCC(CO)CCCCNC(*)=O]

and is attached to the 3′ carbon of the 3′ terminal nucleotide of the antisense strand.

In embodiments, -L³-L⁴- is independently

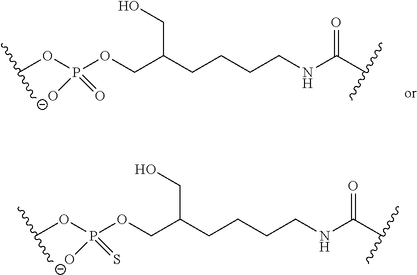 [SMILES: *OP(=O)([O-])OCC(CO)CCCCNC(*)=O ; *OP([O-])(=S)OCC(CO)CCCCNC(*)=O]

that is attached to the 3′ carbon of the 3′ terminal nucleotide of the sense strand.

In embodiments, -L³-L⁴- is independently

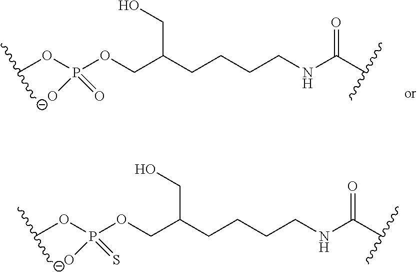 [SMILES: *OP(=O)([O-])OCC(CO)CCCCNC(*)=O ; *OP([O-])(=S)OCC(CO)CCCCNC(*)=O]

that is attached to the 3′ carbon of the 3′ terminal nucleotide of the antisense strand.

In embodiments, -L³-L⁴- is independently

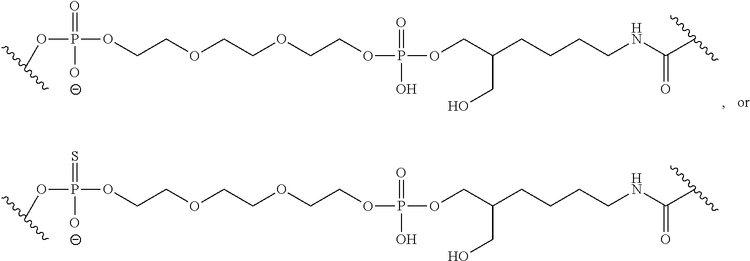 [SMILES: *OP(=O)([O-])OCCOCCOCCOP(=O)(O)OCC(CO)CCCCNC(*)=O ; *OP([O-])(=S)OCCOCCOCCOP(=O)(O)OCC(CO)CCCCNC(*)=O]

that is attached to the 3′ carbon of the 3′ terminal nucleotide of the sense strand.

In embodiments, -L³-L⁴- is independently

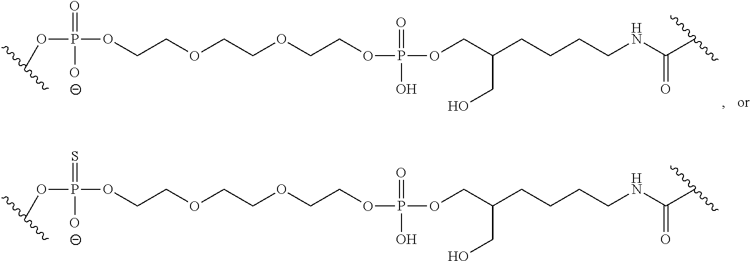 [SMILES: *OP(=O)([O-])OCCOCCOCCOP(=O)(O)OCC(CO)CCCCNC(*)=O ; *OP([O-])(=S)OCCOCCOCCOP(=O)(O)OCC(CO)CCCCNC(*)=O]

that is attached to the 3′ carbon of the 3′ terminal nucleotide of the antisense strand.

In embodiments, an -L³-L⁴- is independently

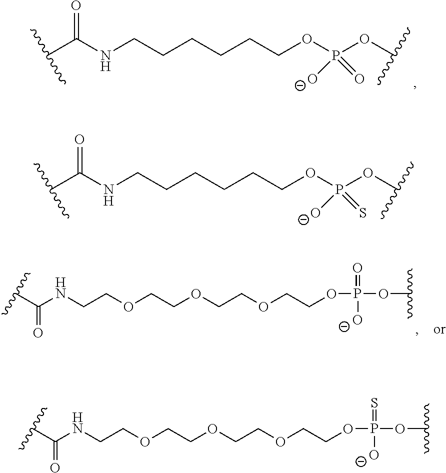 [SMILES: *OP(=O)([O-])OCCCCCCNC(*)=O ; *OP([O-])(=S)OCCCCCCNC(*)=O ; *OP(=O)([O-])OCCOCCOCCOCCNC(*)=O ; *OP([O-])(=S)OCCOCCOCCOCCNC(*)=O]

and is attached to the 5′ carbon of the 5′ terminal nucleotide of the sense strand.

In embodiments, an -L³-L⁴- is independently

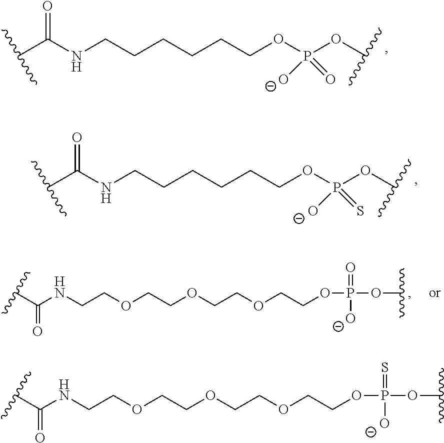 [SMILES: *OP(=O)([O-])OCCCCCCNC(*)=O ; *OP([O-])(=S)OCCCCCCNC(*)=O ; *OP(=O)([O-])OCCOCCOCCOCCNC(*)=O ; *OP([O-])(=S)OCCOCCOCCOCCNC(*)=O]

and is attached to the 5′ carbon of the 5′ terminal nucleotide of the antisense strand.

In embodiments, an -L³-L⁴- is independently

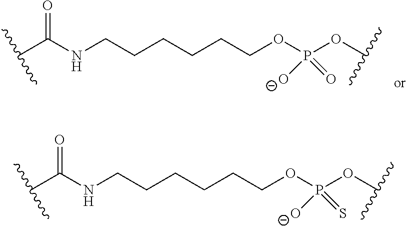 [SMILES: *OP(=O)([O-])OCCCCCCNC(*)=O ; *OP([O-])(=S)OCCCCCCNC(*)=O]

that is attached to the 5′ carbon of the 5′ terminal nucleotide of the sense strand.

In embodiments, an -L³-L⁴- is independently

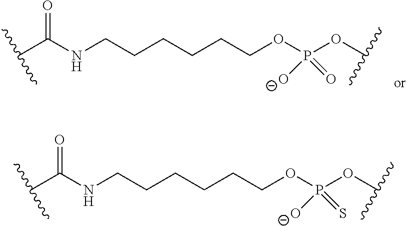 [SMILES: *OP(=O)([O-])OCCCCCCNC(*)=O ; *OP([O-])(=S)OCCCCCCNC(*)=O]

that is attached to the 5′ carbon of the 5′ terminal nucleotide of the antisense strand.

In embodiments, an -L³-L⁴- is independently

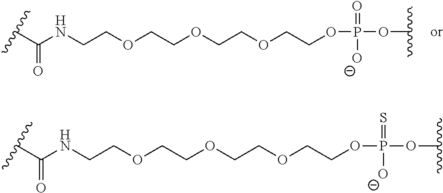 [SMILES: *OP(=O)([O-])OCCOCCOCCOCCNC(*)=O ; *OP([O-])(=S)OCCOCCOCCOCCNC(*)=O]

that is attached to 5′ carbon of the 5′ terminal nucleotide of the sense strand.

In embodiments, an -L³-L⁴- is independently

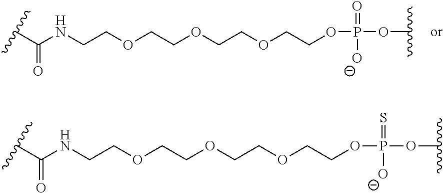 [SMILES: *OP(=O)([O-])OCCOCCOCCOCCNC(*)=O ; *OP([O-])(=S)OCCOCCOCCOCCNC(*)=O]

that is attached to the 5′ carbon of the 5′ terminal nucleotide of the antisense strand.

In embodiments, an -L³-L⁴- is independently attached to a nucleobase of the sense strand. In embodiments, an -L³-L⁴- is independently

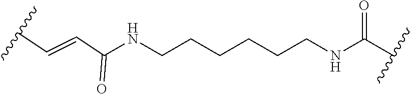 [SMILES: */C=C/C(=O)NCCCCCCNC(*)=O]

and is attached to a nucleobase of the sense strand.

In embodiments, an -L³-L⁴- is independently

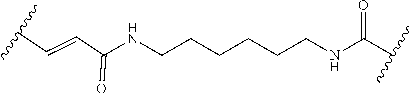 [SMILES: */C=C/C(=O)NCCCCCCNC(*)=O]

and is attached to a nucleobase of the antisense strand.

In embodiments, -L³-L⁴- is independently

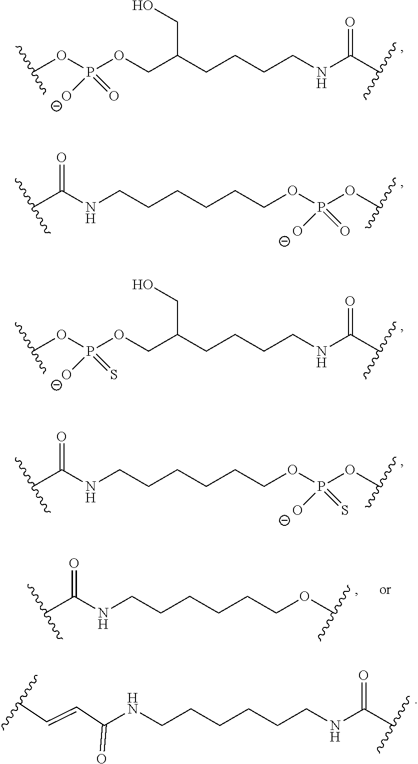 [SMILES: *OP(=O)([O-])OCC(CO)CCCCNC(*)=O ; *OP(=O)([O-])OCCCCCCNC(*)=O ; *OP([O-])(=S)OCC(CO)CCCCNC(*)=O ; *OP([O-])(=S)OCCCCCCNC(*)=O ; *OCCCCCCNC(*)=O ; */C=C/C(=O)NCCCCCCNC(*)=O]

In embodiments, -L³-L⁴- is independently

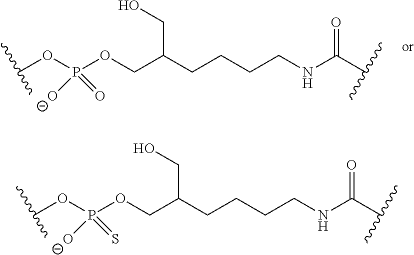 [SMILES: *OP(=O)([O-])OCC(CO)CCCCNC(*)=O ; *OP([O-])(=S)OCC(CO)CCCCNC(*)=O]

that is attached to the 3′ carbon of the 3′ terminal nucleotide of the sense strand.

In embodiments, -L³-L⁴- is independently

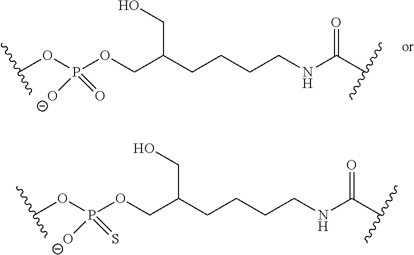 [SMILES: *OP(=O)([O-])OCC(CO)CCCCNC(*)=O ; *OP([O-])(=S)OCC(CO)CCCCNC(*)=O]

that is attached to the 3′ carbon of the 3′ terminal nucleotide of the antisense strand.

In embodiments, -L³-L⁴- is independently

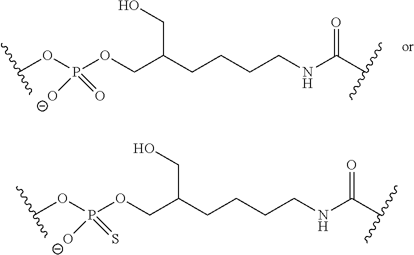 [SMILES: *OP(=O)([O-])OCC(CO)CCCCNC(*)=O ; *OP([O-])(=S)OCC(CO)CCCCNC(*)=O]

that is attached to the 5′ carbon of the 5′ terminal nucleotide of the sense strand.

In embodiments, -L³-L⁴- is independently

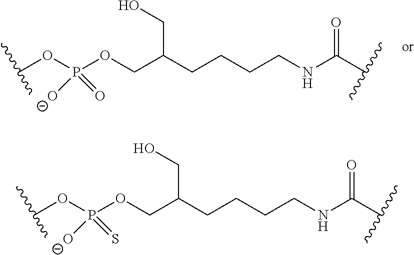 [SMILES: *OP(=O)([O-])OCC(CO)CCCCNC(*)=O ; *OP([O-])(=S)OCC(CO)CCCCNC(*)=O]

that is attached to the 5′ carbon of the 5′ terminal nucleotide of the antisense strand.

In embodiments, -L³-L⁴- is independently

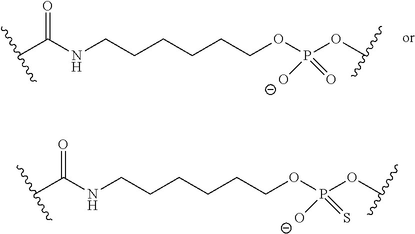 [SMILES: *OP(=O)([O-])OCCCCCCNC(*)=O ; *OP([O-])(=S)OCCCCCCNC(*)=O]

that is attached to the 3′ carbon of the 3′ terminal nucleotide of the sense strand.

In embodiments, -L³-L⁴- is independently

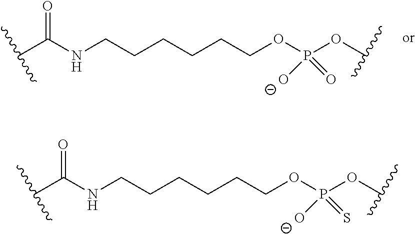 [SMILES: *OP(=O)([O-])OCCCCCCNC(*)=O ; *OP([O-])(=S)OCCCCCCNC(*)=O]

that is attached to the 3′ carbon of the 3′ terminal nucleotide of the antisense strand.

In embodiments, -L³-L⁴- is independently

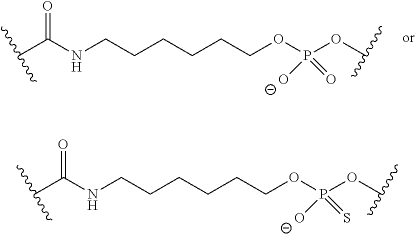 [SMILES: *OP(=O)([O-])OCCCCCCNC(*)=O ; *OP([O-])(=S)OCCCCCCNC(*)=O]

that is attached to the 5′ carbon of the 5′ terminal nucleotide of the sense strand.

In embodiments, -L³-L⁴- is independently

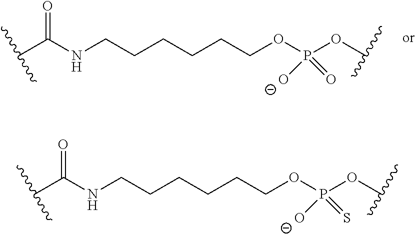 [SMILES: *OP(=O)([O-])OCCCCCCNC(*)=O ; *OP([O-])(=S)OCCCCCCNC(*)=O]

that is attached to the 5′ carbon of the 5′ terminal nucleotide of the antisense strand.

In embodiments, -L³-L⁴- is independently

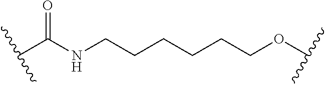 [SMILES: *OCCCCCCNC(*)=O]

that is attached to the 3′ carbon of the 3′ terminal nucleotide of the sense strand.

In embodiments, -L³-L⁴- is independently

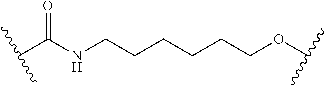 [SMILES: *OCCCCCCNC(*)=O]

that is attached to the 3′ carbon of the 3′ terminal nucleotide of the antisense strand.

In embodiments, -L³-L⁴- is independently

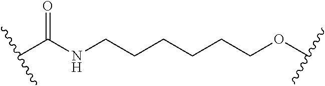 [SMILES: *OCCCCCCNC(*)=O]

and is attached to the 5′ carbon of the 5′ terminal nucleotide of the sense strand.

In embodiments, -L³-L⁴- is independently

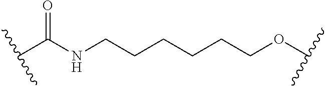 [SMILES: *OCCCCCCNC(*)=O]

and is attached to the 5′ carbon of the 5′ terminal nucleotide of the antisense strand.

In embodiments, -L³-L⁴- is independently

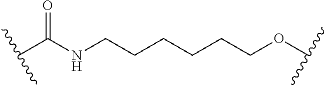 [SMILES: *OCCCCCCNC(*)=O]

and is attached to a 2′ carbon of a nucleotide of the sense strand.

In embodiments, -L³-L⁴- is independently

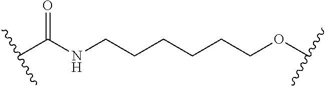 [SMILES: *OCCCCCCNC(*)=O]

and is attached to a 2′ carbon of a nucleotide of the antisense strand.

In embodiments, -L³-L⁴- is independently

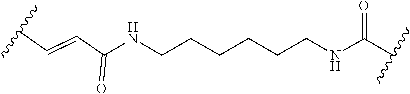 [SMILES: */C=C/C(=O)NCCCCCCNC(*)=O]

and is attached to a 2′ carbon of a nucleotide of the sense strand.

In embodiments, -L³-L⁴- is independently

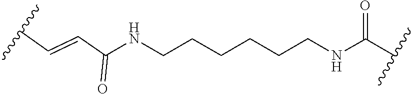 [SMILES: */C=C/C(=O)NCCCCCCNC(*)=O]

and is attached to a 2′ carbon of a nucleotide of the antisense strand.

In embodiments, -L³-L⁴- is independently

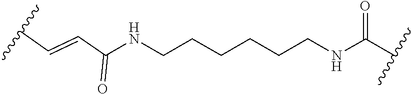 [SMILES: */C=C/C(=O)NCCCCCCNC(*)=O]

and is attached to a nucleobase of the sense strand.

In embodiments, -L³-L⁴- is independently

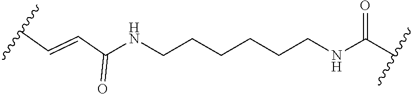 [SMILES: */C=C/C(=O)NCCCCCCNC(*)=O]

and is attached to a nucleobase of the antisense strand.

In embodiments, R³ is independently hydrogen, —NH₂, —OH, —SH, —C(O)H, —C(O)NH₂, —NHC(O)H, —NHC(O)OH, —NHC(O)NH₂, —C(O)OH, —OC(O)H, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. In embodiments, R³ is independently hydrogen. In embodiments, R³ is independently —NH₂. In embodiments, R³ is independently —OH. In embodiments, R³ is independently —SH. In embodiments, R³ is independently —C(O)H. In embodiments, R³ is independently —C(O)NH₂. In embodiments, R³ is independently —NHC(O)H. In embodiments, R³ is independently —NHC(O)OH. In embodiments, R³ is independently —NHC(O)NH₂. In embodiments, R³ is independently —C(O)OH. In embodiments, R³ is independently —OC(O)H. In embodiments, R³ is independently —N₃.

In embodiments, R³ is independently substituted or unsubstituted alkyl (e.g., C₁-C₂₀, C₁-C₁₂, C₁-C₈, C₁-C₆, C₁-C₄, or C₁-C₂). In embodiments, R³ is independently substituted or unsubstituted C₁-C₂₀ alkyl. In embodiments, R³ is independently substituted C₁-C₂₀ alkyl. In embodiments, R³ is independently unsubstituted C₁-C₂₀ alkyl. In embodiments, R³ is independently substituted or unsubstituted C₁-C₁₂ alkyl. In embodiments, R³ is independently substituted C₁-C₁₂ alkyl. In embodiments, R³ is independently unsubstituted C₁-C₁₂ alkyl. In embodiments, R³ is independently substituted or unsubstituted C₁-C₈ alkyl. In embodiments, R³ is independently substituted C₁-C₈ alkyl. In embodiments, R³ is independently unsubstituted C₁-C₈ alkyl. In embodiments, R³ is independently substituted or unsubstituted C₁-C₆ alkyl. In embodiments, R³ is independently substituted C₁-C₆ alkyl. In embodiments, R³ is independently unsubstituted C₁-C₆ alkyl. In embodiments, R³ is independently substituted or unsubstituted C₁-C₄ alkyl. In embodiments, R³ is independently substituted C₁-C₄ alkyl. In embodiments, R³ is independently unsubstituted C₁-C₄ alkyl. In embodiments, R³ is independently substituted or unsubstituted ethyl. In embodiments, R³ is independently substituted ethyl. In embodiments, R³ is independently unsubstituted ethyl. In embodiments, R³ is independently substituted or unsubstituted methyl. In embodiments, R³ is independently substituted methyl. In embodiments, R³ is independently unsubstituted methyl.

In embodiments, L⁶ is independently —NHC(O)—. In embodiments, L⁶ is independently —C(O)NH—. In embodiments, L⁶ is independently substituted or unsubstituted alkylene. In embodiments, L⁶ is independently substituted or unsubstituted heteroalkylene.

In embodiments, L⁶ is independently substituted or unsubstituted alkylene (e.g., C₁-C₂₀, C₁-C₁₂, C₁-C₈, C₁-C₆, C₁-C₄, or C₁-C₂). In embodiments, L⁶ is independently substituted alkylene (e.g., C₁-C₂₀, C₁-C₁₂, C₁-C₈, C₁-C₆, C₁-C₄, or C₁-C₂). In embodiments, L⁶ is independently unsubstituted alkylene (e.g., C₁-C₂₀, C₁-C₁₂, C₁-C₈, C₁-C₆, C₁-C₄, or C₁-C₂). In embodiments, L⁶ is independently substituted or unsubstituted C₁-C₂₀ alkylene. In embodiments, L⁶ is independently substituted C₁-C₂₀ alkylene. In embodiments, L⁶ is independently unsubstituted C₁-C₂₀ alkylene. In embodiments, L⁶ is independently substituted or unsubstituted C₁-C₁₂ alkylene. In embodiments, L⁶ is independently substituted C₁-C₁₂ alkylene. In embodiments, L⁶ is independently unsubstituted C₁-C₁₂ alkylene. In embodiments, L⁶ is independently substituted or unsubstituted C₁-C₈ alkylene. In embodiments, L⁶ is independently substituted C₁-C₈ alkylene. In embodiments, L⁶ is independently unsubstituted C₁-C₈ alkylene. In embodiments, L⁶ is independently substituted or unsubstituted C₁-C₆ alkylene. In embodiments, L⁶ is independently substituted C₁-C₆ alkylene. In embodiments, L⁶ is independently unsubstituted C₁-C₆ alkylene. In embodiments, L⁶ is independently substituted or unsubstituted C₁-C₄ alkylene. In embodiments, L⁶ is independently substituted C₁-C₄ alkylene. In embodiments, L⁶ is independently unsubstituted C₁-C₄ alkylene. In embodiments, L⁶ is independently substituted or unsubstituted ethylene. In embodiments, L⁶ is independently substituted ethylene. In embodiments, L⁶ is independently unsubstituted ethylene. In embodiments, L⁶ is independently substituted or unsubstituted methylene. In embodiments, L⁶ is independently substituted methylene. In embodiments, L⁶ is independently unsubstituted methylene.

In embodiments, L⁶ is independently substituted or unsubstituted heteroalkylene (e.g., 2 to 20 membered, 2 to 12 membered, 2 to 8 membered, 2 to 6 membered, 4 to 6 membered, 2 to 3 membered, or 4 to 5 membered). In embodiments, L⁶ is independently substituted heteroalkylene (e.g., 2 to 20 membered, 2 to 12 membered, 2 to 8 membered, 2 to 6 membered, 4 to 6 membered, 2 to 3 membered, or 4 to 5 membered). In embodiments, L⁶ is independently unsubstituted heteroalkylene (e.g., 2 to 20 membered, 2 to 12 membered, 2 to 8 membered, 2 to 6 membered, 4 to 6 membered, 2 to 3 membered, or 4 to 5 membered). In embodiments, L⁶ is independently substituted or unsubstituted 2 to 20 membered heteroalkylene. In embodiments, L⁶ is independently substituted 2 to 20 membered heteroalkylene. In embodiments, L⁶ is independently unsubstituted 2 to 20 membered heteroalkylene. In embodiments, L⁶ is independently substituted or unsubstituted 2 to 8 membered heteroalkylene. In embodiments, L⁶ is independently substituted 2 to 8 membered heteroalkylene. In embodiments, L⁶ is independently unsubstituted 2 to 8 membered heteroalkylene. In embodiments, L⁶ is independently substituted or unsubstituted 2 to 6 membered heteroalkylene. In embodiments, L⁶ is independently substituted 2 to 6 membered heteroalkylene. In embodiments, L⁶ is independently unsubstituted 2 to 6 membered heteroalkylene. In embodiments, L⁶ is independently substituted or unsubstituted 4 to 6 membered heteroalkylene. In embodiments, L⁶ is independently substituted 4 to 6 membered heteroalkylene. In embodiments, L⁶ is independently unsubstituted 4 to 6 membered heteroalkylene. In embodiments, L⁶ is independently substituted or unsubstituted 2 to 3 membered heteroalkylene. In embodiments, L⁶ is independently substituted 2 to 3 membered heteroalkylene. In embodiments, L⁶ is independently unsubstituted 2 to 3 membered heteroalkylene. In embodiments, L⁶ is independently substituted or unsubstituted 4 to 5 membered heteroalkylene. In embodiments, L⁶ is independently substituted 4 to 5 membered heteroalkylene. In embodiments, L⁶ is independently unsubstituted 4 to 5 membered heteroalkylene.

In embodiments, L^{6A} is independently a bond or unsubstituted alkylene; L^{6B} is independently a bond, —NHC(O)—, or unsubstituted arylene; L^{6C} is independently a bond, unsubstituted alkylene, or unsubstituted arylene; L^{6D} is independently a bond or unsubstituted alkylene; and L^{6E} is independently a bond or —NHC(O)—. In embodiments, L^{6A} is independently a bond or unsubstituted alkylene. In embodiments, L^{6B} is independently a bond, —NHC(O)—, or unsubstituted arylene. In embodiments, L^{6C} is independently a bond, unsubstituted alkylene, or unsubstituted arylene. In embodiments, L^{6D} is independently a bond or unsubstituted alkylene. In embodiments, L^{6E} is independently a bond or —NHC(O)—.

In embodiments, L^{6A} is independently a bond or unsubstituted alkylene (e.g., C₁-C₂₀, C₁-C₁₂, C₁-C₈, C₁-C₆, C₁-C₄, or C₁-C₂). In embodiments, L^{6A} is independently unsubstituted C₁-C₂₀ alkylene. In embodiments, L^{6A} is independently unsubstituted C₁-C₁₂ alkylene. In embodiments, L^{6A} is independently unsubstituted C₁-C₈ alkylene. In embodiments, L^{6A} is independently unsubstituted C₁-C₆ alkylene. In embodiments, L^{6A} is independently unsubstituted C₁-C₄ alkylene. In embodiments, L^{6A} is independently unsubstituted ethylene. In embodiments, L^{6A} is independently unsubstituted methylene. In embodiments, L^{6A} is independently a bond.

In embodiments, L^{6B} is independently a bond. In embodiments, L^{6B} is independently —NHC(O)—. In embodiments, L^{6B} is independently unsubstituted arylene (e.g., C₆-C₁₂, C₆-C₁₀, or phenyl). In embodiments, L^{6B} is independently unsubstituted C₆-C₁₂ arylene. In embodiments, L^{6B} is independently unsubstituted C₆-C₁₀ arylene. In embodiments, L^{6B} is independently unsubstituted phenylene. In embodiments, L^{6B} is independently unsubstituted naphthylene. In embodiments, L^{6B} is independently unsubstituted biphenylene.

In embodiments, L⁶C is independently a bond or unsubstituted alkylene (e.g., C₁-C₂₀, C₁-C₁₂, C₁-C₈, C₁-C₆, C₁-C₄, or C₁-C₂). In embodiments, L⁶C is independently unsubstituted C₁-C₂₀ alkylene. In embodiments, L⁶C is independently unsubstituted C₁-C₁₂ alkylene. In embodiments, L⁶C is independently unsubstituted C₁-C₈ alkylene. L⁶C is independently unsubstituted C₂-C₈ alkynylene. In embodiments, L⁶C is independently unsubstituted C₁-C₆ alkylene. In embodiments, L⁶C is independently unsubstituted C₁-C₄ alkylene. In embodiments, L⁶C is independently unsubstituted ethylene. In embodiments, L⁶C is independently unsubstituted methylene. In embodiments, L⁶C is independently a bond or unsubstituted alkynylene (e.g., C₂-C₂₀, C₂-C₁₂, C₂-C₈, C₂-C₆, C₂-C₄, or C₂-C₂). In embodiments, L⁶C is independently unsubstituted C₂-C₂₀ alkynylene. In embodiments, L^{6C} is independently unsubstituted C₂-C₁₂ alkynylene. In embodiments, L^{6C} is independently unsubstituted C₂-C₈ alkynylene. In embodiments, L^{6C} is independently unsubstituted C₂-C₆ alkynylene. In embodiments, L^{6C} is independently unsubstituted C₂-C₄ alkynylene. In embodiments, L^{6C} is independently unsubstituted ethynylene. In embodiments, L^{6C} is independently unsubstituted arylene (e.g., C₆-C₁₂, C₆-C₁₀, or phenyl). In embodiments, L^{6C} is independently unsubstituted C₆-C₁₂ arylene. In embodiments, L^{6C} is independently unsubstituted C₆-C₁₀ arylene. In embodiments, L^{6C} is independently unsubstituted phenylene. In embodiments, L^{6C} is independently unsubstituted naphthylene. In embodiments, L^{6C} is independently a bond.

In embodiments, L^{6D} is independently a bond or unsubstituted alkylene (e.g., C₁-C₂₀, C₁-C₁₂, C₁-C₈, C₁-C₆, C₁-C₄, or C₁-C₂). In embodiments, L^{6D} is independently unsubstituted C₁-C₂₀ alkylene. In embodiments, L^{6D} is independently unsubstituted C₁-C₁₂ alkylene. In embodiments, L^{6A} is independently unsubstituted C₁-C₈ alkylene. In embodiments, L^{6D} is independently unsubstituted C₁-C₆ alkylene. In embodiments, L^{6D} is independently unsubstituted C₁-C₄ alkylene. In embodiments, L^{6D} is independently unsubstituted ethylene. In embodiments, L^{6D} is independently unsubstituted methylene. In embodiments, L^{6D} is independently a bond.

In embodiments, L^{6E} is independently a bond. In embodiments, L^{6E} is independently —NHC(O)—.

In embodiments, L^{6A} is independently a bond or unsubstituted C₁-C₈ alkylene. In embodiments, L^{6B} is independently a bond, —NHC(O)—, or unsubstituted phenylene. In embodiments, L^{6C} is independently a bond, unsubstituted C₂-C₈ alkynylene, or unsubstituted phenylene. In embodiments, L^{6D} is independently a bond or unsubstituted C₁-C₈ alkylene. In embodiments, L^{6E} is independently a bond or —NHC(O)—.

In embodiments, L⁶ is independently a bond,

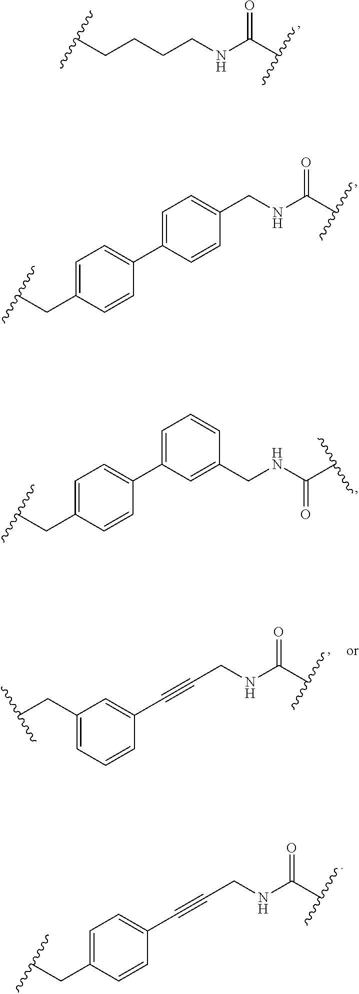 [SMILES: *CCCCNC(*)=O ; *Cc1ccc(-c2ccc(CNC(*)=O)cc2)cc1 ; *Cc1ccc(-c2cccc(CNC(*)=O)c2)cc1 ; *Cc1cccc(C#CCNC(*)=O)c1 ; *Cc1ccc(C#CCNC(*)=O)cc1]

In embodiments, L⁶ is independently a bond. In embodiments, L⁶ is independently

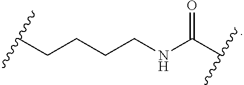 [SMILES: *CCCCNC(*)=O]

In embodiments, L⁶ is independently

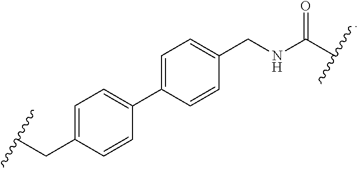 [SMILES: *Cc1ccc(-c2ccc(CNC(*)=O)cc2)cc1]

In embodiments, L is independently

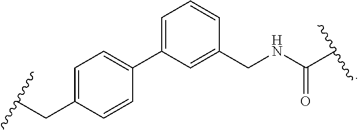 [SMILES: *Cc1ccc(-c2cccc(CNC(*)=O)c2)cc1]

In embodiments, L⁶ is independently

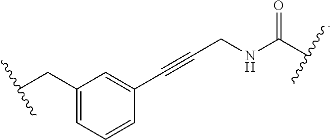 [SMILES: *Cc1cccc(C#CCNC(*)=O)c1]

In embodiments, L⁶ is independently

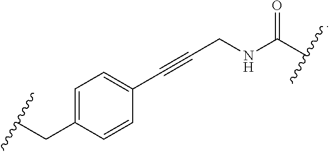 [SMILES: *Cc1ccc(C#CCNC(*)=O)cc1]

In embodiments, L⁵ is independently —NHC(O)—. In embodiments, L⁵ is independently —C(O)NH—. In embodiments, L⁵ is independently substituted or unsubstituted alkylene. In embodiments, L⁵ is independently substituted or unsubstituted heteroalkylene.

In embodiments, L⁵ is independently substituted or unsubstituted alkylene (e.g., C₁-C₂₀, C₁-C₁₂, C₁-C₈, C₁-C₆, C₁-C₄, or C₁-C₂). In embodiments, L⁵ is independently substituted alkylene (e.g., C₁-C₂₀, C₁-C₁₂, C₁-C₈, C₁-C₆, C₁-C₄, or C₁-C₂). In embodiments, L⁵ is independently unsubstituted alkylene (e.g., C₁-C₂₀, C₁-C₁₂, C₁-C₈, C₁-C₆, C₁-C₄, or C₁-C₂). In embodiments, L⁵ is independently substituted or unsubstituted C₁-C₂₀ alkylene. In embodiments, L⁵ is independently substituted C₁-C₂₀ alkylene. In embodiments, L⁵ is independently unsubstituted C₁-C₂₀ alkylene. In embodiments, L⁵ is independently substituted or unsubstituted C₁-C₁₂ alkylene. In embodiments, L⁵ is independently substituted C₁-C₁₂ alkylene. In embodiments, L⁵ is independently unsubstituted C₁-C₁₂ alkylene. In embodiments, L⁵ is independently substituted or unsubstituted C₁-C₈ alkylene. In embodiments, L⁵ is independently substituted C₁-C₈ alkylene. In embodiments, L⁵ is independently unsubstituted C₁-C₈ alkylene. In embodiments, L⁵ is independently substituted or unsubstituted C₁-C₆ alkylene. In embodiments, L⁵ is independently substituted C₁-C₆ alkylene. In embodiments, L⁵ is independently unsubstituted C₁-C₆ alkylene. In embodiments, L⁵ is independently substituted or unsubstituted C₁-C₄ alkylene. In embodiments, L⁵ is independently substituted C₁-C₄ alkylene. In embodiments, L⁵ is independently unsubstituted C₁-C₄ alkylene. In embodiments, L⁵ is independently substituted or unsubstituted ethylene. In embodiments, L⁵ is independently substituted ethylene. In embodiments, L⁵ is independently unsubstituted ethylene. In embodiments, L⁵ is independently substituted or unsubstituted methylene. In embodiments, L⁵ is independently substituted methylene. In embodiments, L⁵ is independently unsubstituted methylene.

In embodiments, L⁵ is independently substituted or unsubstituted heteroalkylene (e.g., 2 to 20 membered, 2 to 12 membered, 2 to 8 membered, 2 to 6 membered, 4 to 6 membered, 2 to 3 membered, or 4 to 5 membered). In embodiments, L⁵ is independently substituted heteroalkylene (e.g., 2 to 20 membered, 2 to 12 membered, 2 to 8 membered, 2 to 6 membered, 4 to 6 membered, 2 to 3 membered, or 4 to 5 membered). In embodiments, L⁵ is independently unsubstituted heteroalkylene (e.g., 2 to 20 membered, 2 to 12 membered, 2 to 8 membered, 2 to 6 membered, 4 to 6 membered, 2 to 3 membered, or 4 to 5 membered). In embodiments, L⁵ is independently substituted or unsubstituted 2 to 20 membered heteroalkylene. In embodiments, L⁵ is independently substituted 2 to 20 membered heteroalkylene. In embodiments, L⁵ is independently unsubstituted 2 to 20 membered heteroalkylene. In embodiments, L⁵ is independently substituted or unsubstituted 2 to 8 membered heteroalkylene. In embodiments, L⁵ is independently substituted 2 to 8 membered heteroalkylene. In embodiments, L⁵ is independently unsubstituted 2 to 8 membered heteroalkylene. In embodiments, L⁵ is independently substituted or unsubstituted 2 to 6 membered heteroalkylene. In embodiments, L⁵ is independently substituted 2 to 6 membered heteroalkylene. In embodiments, L⁵ is independently unsubstituted 2 to 6 membered heteroalkylene. In embodiments, L⁵ is independently substituted or unsubstituted 4 to 6 membered heteroalkylene. In embodiments, L⁵ is independently substituted 4 to 6 membered heteroalkylene. In embodiments, L⁵ is independently unsubstituted 4 to 6 membered heteroalkylene. In embodiments, L⁵ is independently substituted or unsubstituted 2 to 3 membered heteroalkylene. In embodiments, L⁵ is independently substituted 2 to 3 membered heteroalkylene. In embodiments, L⁵ is independently unsubstituted 2 to 3 membered heteroalkylene. In embodiments, L⁵ is independently substituted or unsubstituted 4 to 5 membered heteroalkylene. In embodiments, L⁵ is independently substituted 4 to 5 membered heteroalkylene. In embodiments, L⁵ is independently unsubstituted 4 to 5 membered heteroalkylene.

In embodiments, L^{5A} is independently a bond or unsubstituted alkylene; L^{5B} is independently a bond, —NHC(O)—, or unsubstituted arylene; L^{5C} is independently a bond, unsubstituted alkylene, or unsubstituted arylene; L^{5D} is independently a bond or unsubstituted alkylene; and L^{5E} is independently a bond or —NHC(O)—. In embodiments, L^{5A} is independently a bond or unsubstituted alkylene. In embodiments, L^{5B} is independently a bond, —NHC(O)—, or unsubstituted arylene. In embodiments, L^{5C} is independently a bond, unsubstituted alkylene, or unsubstituted arylene. In embodiments, L^{5D} is independently a bond or unsubstituted alkylene. In embodiments, L^{5E} is independently a bond or —NHC(O)—.

In embodiments, L^{5A} is independently a bond or unsubstituted alkylene (e.g., C₁-C₂₀, C₁-C₁₂, C₁-C₈, C₁-C₆, C₁-C₄, or C₁-C₂). In embodiments, L^{5A} is independently unsubstituted C₁-C₂₀ alkylene. In embodiments, L^{5A} is independently unsubstituted C₁-C₁₂ alkylene. In embodiments, L^{5A} is independently unsubstituted C₁-C₈ alkylene. In embodiments, L^{5A} is independently unsubstituted C₁-C₆ alkylene. In embodiments, L^{5A} is independently unsubstituted C₁-C₄ alkylene. In embodiments, L^{5A} is independently unsubstituted ethylene. In embodiments, L^{5A} is independently unsubstituted methylene. In embodiments, L^{5A} is independently a bond.

In embodiments, L^{5B} is independently a bond. In embodiments, L^{5B} is independently —NHC(O)—. In embodiments, L^{5B} is independently unsubstituted arylene (e.g., C₆-C₁₂, C₆-C₁₀, or phenyl). In embodiments, L^{5B} is independently unsubstituted C₆-C₁₂ arylene. In embodiments, L^{5B} is independently unsubstituted C₆-C₁₀ arylene. In embodiments, L^{5B} is independently unsubstituted phenylene. In embodiments, L^{5B} is independently unsubstituted naphthylene.

In embodiments, L^{5C} is independently a bond or unsubstituted alkylene (e.g., C₁-C₂₀, C₁-C₁₂, C₁-C₈, C₁-C₆, C₁-C₄, or C₁-C₂). In embodiments, L^{5C} is independently unsubstituted C₁-C₂₀ alkylene. In embodiments, L^{5C} is independently unsubstituted C₁-C₁₂ alkylene. In embodiments, L^{5C} is independently unsubstituted C₁-C₈ alkylene. L^{5C} is independently unsubstituted C₂-C₈ alkynylene. In embodiments, L^{5C} is independently unsubstituted C₁-C₆ alkylene. In embodiments, L^{5C} is independently unsubstituted C₁-C₄ alkylene. In embodiments, L^{5C} is independently unsubstituted ethylene. In embodiments, L^{5C} is independently unsubstituted methylene. In embodiments, L^{5C} is independently a bond or unsubstituted alkynylene (e.g., C₂-C₂₀, C₂-C₁₂, C₂-C₈, C₂-C₆, C₂-C₄, or C₂-C₂). In embodiments, L^{5C} is independently unsubstituted C₂-C₂₀ alkynylene. In embodiments, L^{5C} is independently unsubstituted C₂-C₁₂ alkynylene. In embodiments, L^{5C} is independently unsubstituted C₂-C₈ alkynylene. In embodiments, L^{5C} is independently unsubstituted C₂-C₆ alkynylene. In embodiments, L^{5C} is independently unsubstituted C₂-C₄ alkynylene. In embodiments, L^{5C} is independently unsubstituted ethynylene. In embodiments, L^{5C} is independently unsubstituted arylene (e.g., C₆-C₁₂, C₆-C₁₀, or phenyl). In embodiments, L^{5C} is independently unsubstituted C₆-C₁₂ arylene. In embodiments, L^{5C} is independently unsubstituted C₆-C₁₀ arylene. In embodiments, L^{5C} is independently unsubstituted phenylene. In embodiments, L^{5C} is independently unsubstituted naphthylene. In embodiments, L^{5C} is independently a bond.

In embodiments, L^{5D} is independently a bond or unsubstituted alkylene (e.g., C₁-C₂₀, C₁-C₁₂, C₁-C₈, C₁-C₆, C₁-C₄, or C₁-C₂). In embodiments, L^{5D} is independently unsubstituted C₁-C₂₀ alkylene. In embodiments, L^{5D} is independently unsubstituted C₁-C₁₁ alkylene. In embodiments, L^{5A} is independently unsubstituted C₁-C₈ alkylene. In embodiments, L^{5D} is independently unsubstituted C₁-C₆ alkylene. In embodiments, L^{5D} is independently unsubstituted C₁-C₄ alkylene. In embodiments, L^{5D} is independently unsubstituted ethylene. In embodiments, L^{5D} is independently unsubstituted methylene. In embodiments, L^{5D} is independently a bond.

In embodiments, L^{5E} is independently a bond. In embodiments, L^{5E} is independently —NHC(O)—.

In embodiments, L^{5A} is independently a bond or unsubstituted C₁-C₈ alkylene. In embodiments, L^{5B} is independently a bond, —NHC(O)—, or unsubstituted phenylene. In embodiments, L^{5C} is independently a bond, unsubstituted C₂-C₈ alkynylene, or unsubstituted phenylene. In embodiments, L^{5D} is independently a bond or unsubstituted C₁-C₈ alkylene. In embodiments, L^{5E} is independently a bond or —NHC(O)—.

In embodiments, L⁵ is independently a bond,

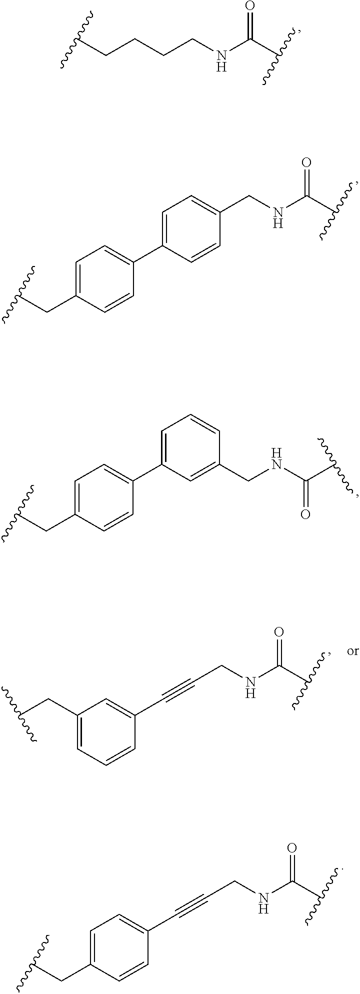 [SMILES: *CCCCNC(*)=O ; *Cc1ccc(-c2ccc(CNC(*)=O)cc2)cc1 ; *Cc1ccc(-c2cccc(CNC(*)=O)c2)cc1 ; *Cc1cccc(C#CCNC(*)=O)c1 ; *Cc1ccc(C#CCNC(*)=O)cc1]

In embodiments, L⁵ is independently a bond. In embodiments, L⁵ is independently

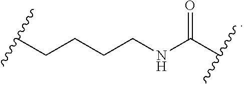 [SMILES: *CCCCNC(*)=O]

In embodiments, L⁵ is independently

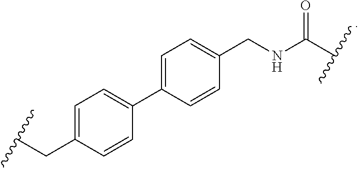 [SMILES: *Cc1ccc(-c2ccc(CNC(*)=O)cc2)cc1]

In embodiments, L⁵ is independently

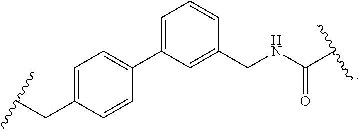 [SMILES: *Cc1ccc(-c2cccc(CNC(*)=O)c2)cc1]

In embodiments, L⁵ is independently

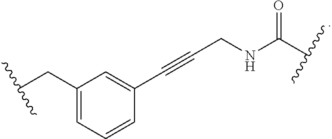 [SMILES: *Cc1cccc(C#CCNC(*)=O)c1]

In embodiments, L⁵ is independently

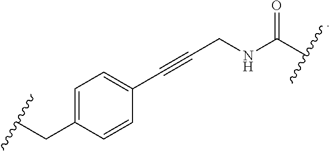 [SMILES: *Cc1ccc(C#CCNC(*)=O)cc1]

In embodiments, R¹ is unsubstituted alkyl (e.g., C₁-C₂₅, C₁-C₂₀, C₁-C₁₇, C₁-C₁₂, C₁-C₈, C₁-C₆, C₁-C₄, or C₁-C₂). In embodiments, R¹ is unsubstituted unbranched alkyl (e.g., C₁-C₂₅, C₁-C₂₀, C₁-C₁₇, C₁-C₁₂, C₁-C₈, C₁-C₆, C₁-C₄, or C₁-C₂). In embodiments, R¹ is unsubstituted unbranched saturated alkyl (e.g., C₁-C₂₅, C₁-C₂₀, C₁-C₁₇, C₁-C₁₂, C₁-C₈, C₁-C₆, C₁-C₄, or C₁-C₂). In embodiments, R¹ is unsubstituted unbranched unsaturated alkyl (e.g., C₁-C₂₅, C₁-C₂₀, C₁-C₁₇, C₁-C₁₂, C₁-C₈, C₁-C₆, C₁-C₄, or C₁-C₂).

In embodiments, R¹ is unsubstituted C₁-C₁₇ alkyl. In embodiments, R¹ is unsubstituted C₁₁-C₁₇ alkyl. In embodiments, R¹ is unsubstituted C₁₃-C₁₇ alkyl. In embodiments, R¹ is unsubstituted C₁₄-C₁₅ alkyl. In embodiments, R¹ is unsubstituted C₁₅ alkyl. In embodiments, R¹ is unsubstituted C₁₄ alkyl.

In embodiments, R¹ is unsubstituted unbranched C₁-C₁₇ alkyl. In embodiments, R¹ is unsubstituted unbranched C₁-C₁₇ alkyl. In embodiments, R¹ is unsubstituted unbranched C₁₃-C₁₇ alkyl. In embodiments, R¹ is unsubstituted unbranched C₁₄-C₁₅ alkyl. In embodiments, R¹ is unsubstituted unbranched C₁₄ alkyl. In embodiments, R¹ is unsubstituted unbranched C₁₅ alkyl.

In embodiments, R¹ is unsubstituted unbranched saturated C₁-C₁₇ alkyl. In embodiments, R¹ is unsubstituted unbranched saturated C₁₁-C₁₇ alkyl. In embodiments, R¹ is unsubstituted unbranched saturated C₁₃-C₁₇ alkyl. In embodiments, R¹ is unsubstituted unbranched saturated C₁₄-C₁₅ alkyl. In embodiments, R¹ is unsubstituted unbranched saturated C₁₄ alkyl. In embodiments, R¹ is unsubstituted unbranched saturated C₁₅ alkyl.

In embodiments, R¹ is unsubstituted unbranched unsaturated C₁-C₁₇ alkyl. In embodiments, R¹ is unsubstituted unbranched unsaturated C₁₁-C₁₇ alkyl. In embodiments, R¹ is unsubstituted unbranched unsaturated C₁₃-C₁₇ alkyl. In embodiments, R¹ is unsubstituted unbranched unsaturated C₁₄-C₁₅ alkyl. In embodiments, R¹ is unsubstituted unbranched unsaturated C₁₄ alkyl. In embodiments, R¹ is unsubstituted unbranched unsaturated C₁₅ alkyl.

In embodiments, R² is unsubstituted alkyl (e.g., C₁-C₂₅, C₁-C₂₀, C₁-C₁₇, C₁-C₁₂, C₁-C₈, C₁-C₆, C₁-C₄, or C₁-C₂). In embodiments, R² is unsubstituted unbranched alkyl (e.g., C₁-C₂₅, C₁-C₂₀, C₁-C₁₇, C₁-C₁₂, C₁-C₈, C₁-C₆, C₁-C₄, or C₁-C₂). In embodiments, R² is unsubstituted unbranched saturated alkyl (e.g., C₁-C₂₅, C₁-C₂₀, C₁-C₁₇, C₁-C₁₂, C₁-C₈, C₁-C₆, C₁-C₄, or C₁-C₂).

In embodiments, R² is unsubstituted unbranched unsaturated alkyl (e.g., C₁-C₂₅, C₁-C₂₀, C₁-C₁₇, C₁-C₁₂, C₁-C₈, C₁-C₆, C₁-C₄, or C₁-C₂).

In embodiments, R² is unsubstituted C₁-C₁₇ alkyl. In embodiments, R² is unsubstituted C₁₁-C₁₇ alkyl. In embodiments, R² is unsubstituted C₁₃-C₁₇ alkyl. In embodiments, R² is unsubstituted C₁₄-C₁₅ alkyl. In embodiments, R² is unsubstituted C₁₄ alkyl. In embodiments, R² is unsubstituted C₁₅ alkyl.

In embodiments, R² is unsubstituted unbranched C₁-C₁₇ alkyl. In embodiments, R² is unsubstituted unbranched C₁₁-C₁₇ alkyl. In embodiments, R² is unsubstituted unbranched C₁₃-C₁₇ alkyl. In embodiments, R² is unsubstituted unbranched C₁₄-C₁₅ alkyl. In embodiments, R² is unsubstituted unbranched C₁₄ alkyl. In embodiments, R² is unsubstituted unbranched C₁₅ alkyl.

In embodiments, R² is unsubstituted unbranched saturated C₁-C₁₇ alkyl. In embodiments, R² is unsubstituted unbranched saturated C₁₁-C₁₇ alkyl. In embodiments, R² is unsubstituted unbranched saturated C₁₃-C₁₇ alkyl. In embodiments, R² is unsubstituted unbranched saturated C₁₄-C₁₅ alkyl. In embodiments, R² is unsubstituted unbranched saturated C₁₄ alkyl. In embodiments, R² is unsubstituted unbranched saturated C₁₅ alkyl.

In embodiments, R² is unsubstituted unbranched unsaturated C₁-C₁₇ alkyl. In embodiments, R² is unsubstituted unbranched unsaturated C₁₁-C₁₇ alkyl. In embodiments, R² is unsubstituted unbranched unsaturated C₁₃-C₁₇ alkyl. In embodiments, R² is unsubstituted unbranched unsaturated C₁₄-C₁₅ alkyl. In embodiments, R² is unsubstituted unbranched unsaturated C₁₄ alkyl. In embodiments, R² is unsubstituted unbranched unsaturated C₁₅ alkyl.

In embodiments, at least one of R¹ and R² is unsubstituted C₁-C₁₉ alkyl. In embodiments, at least one of R¹ and R² is unsubstituted C₉-C₁₉ alkyl. In embodiments, at least one of R¹ and R² is unsubstituted C₁₁-C₁₉ alkyl. In embodiments, at least one of R¹ and R² is unsubstituted C₁₃-C₁₉ alkyl.

In embodiments, R¹ is unsubstituted C₁-C₁₉ alkyl. In embodiments, R¹ is unsubstituted C₉-C₁₉ alkyl. In embodiments, R¹ is unsubstituted C₁₁-C₁₉ alkyl. In embodiments, R¹ is unsubstituted C₁₃-C₁₉ alkyl. In embodiments, R¹ is unsubstituted unbranched C₁-C₁₉ alkyl. In embodiments, R¹ is unsubstituted unbranched C₉-C₁₉ alkyl. In embodiments, R¹ is unsubstituted unbranched C₁₁-C₁₉ alkyl. In embodiments, R¹ is unsubstituted unbranched C₁₃-C₁₉ alkyl. In embodiments, R¹ is unsubstituted unbranched saturated C₁-C₁₉ alkyl. In embodiments, R¹ is unsubstituted unbranched saturated C₉-C₁₉ alkyl. In embodiments, R¹ is unsubstituted unbranched saturated C₁₁-C₁₉ alkyl. In embodiments, R¹ is unsubstituted unbranched saturated C₁₃-C₁₉ alkyl. In embodiments, R¹ is unsubstituted unbranched unsaturated C₁-C₁₉ alkyl. In embodiments, R¹ is unsubstituted unbranched unsaturated C₉-C₁₉ alkyl. In embodiments, R¹ is unsubstituted unbranched unsaturated C₁₁-C₁₉ alkyl. In embodiments, R¹ is unsubstituted unbranched unsaturated C₁₃-C₁₉ alkyl.

In embodiments, R² is unsubstituted C₁-C₁₉ alkyl. In embodiments, R² is unsubstituted C₉-C₁₉ alkyl. In embodiments, R² is unsubstituted C₁₁-C₁₉ alkyl. In embodiments, R² is unsubstituted C₁₃-C₁₉ alkyl. In embodiments, R² is unsubstituted unbranched C₁-C₁₉ alkyl. In embodiments, R² is unsubstituted unbranched C₉-C₁₉ alkyl. In embodiments, R² is unsubstituted unbranched C₁₁-C₁₉ alkyl. In embodiments, R² is unsubstituted unbranched C₁₃-C₁₉ alkyl. In embodiments, R² is unsubstituted unbranched saturated C₁-C₁₉ alkyl. In embodiments, R² is unsubstituted unbranched saturated C₉-C₁₉ alkyl. In embodiments, R² is unsubstituted unbranched saturated C₁₁-C₁₉ alkyl. In embodiments, R² is unsubstituted unbranched saturated C₁₃-C₁₉ alkyl. In embodiments, R² is unsubstituted unbranched unsaturated C₁-C₁₉ alkyl. In embodiments, R² is unsubstituted unbranched unsaturated C₉-C₁₉ alkyl. In embodiments, R² is unsubstituted unbranched unsaturated C₁₁-C₁₉ alkyl. In embodiments, R² is unsubstituted unbranched unsaturated C₁₃-C₁₉ alkyl.

L^{1A} is independently a bond, —N(R²⁰)—, —O—, —S—, —C(O)—, —N(R²⁰)C(O)—, —C(O)N(R²¹)—, —N(R²⁰)C(O)N(R²¹)—, —C(O)O—, —OC(O)—, —N(R²⁰)C(O)O—, —OC(O)N(R²¹)—, —OPO₂—O—, —O—P(O)(S)—O—, —O—P(O)(R²²)—O—, —O—P(S)(R²²)—O—, —O—P(O)(NR²⁰R²¹)—N—, —O—P(S)(NR²⁰R²¹)—N—, —O—P(O)(NR²⁰R²¹)—O—, —O—P(S)(NR²⁰R²¹)—O—, —P(O)(NR²⁰R²¹)—N—, —P(S)(NR²⁰R²¹)—N—, —P(O)(NR²⁰R²¹)—O—, —P(S)(NR²⁰R²¹)—O—, —S—S—, substituted (e.g., substituted with a substituent group, a size-limited substituent group, or lower substituent group) or unsubstituted alkylene (e.g., C₁-C₂₀, C₁-C₁₂, C₁-C₈, C₁-C₆, C₁-C₄, or C₁-C₂), substituted (e.g., substituted with a substituent group, a size-limited substituent group, or lower substituent group) or unsubstituted heteroalkylene (e.g., 2 to 20 membered, 2 to 12 membered, 2 to 8 membered, 2 to 6 membered, 4 to 6 membered, 2 to 3 membered, or 4 to 5 membered), substituted (e.g., substituted with a substituent group, a size-limited substituent group, or lower substituent group) or unsubstituted cycloalkylene (e.g., C₃-C₁₀, C₃-C₈, C₃-C₆, C₄-C₆, or C₅-C₆), substituted (e.g., substituted with a substituent group, a size-limited substituent group, or lower substituent group) or unsubstituted heterocycloalkylene (e.g., 3 to 10 membered, 3 to 8 membered, 3 to 6 membered, 4 to 6 membered, 4 to 5 membered, or 5 to 6 membered), substituted (e.g., substituted with a substituent group, a size-limited substituent group, or lower substituent group) or unsubstituted arylene (e.g., C₆-C₁₂, C₆-C₁₀, or phenyl), or substituted (e.g., substituted with a substituent group, a size-limited substituent group, or lower substituent group) or unsubstituted heteroarylene (e.g., 5 to 12 membered, 5 to 10 membered, 5 to 9 membered, or 5 to 6 membered). In embodiments, L^{1A} is independently a bond, —N(R²⁰)—, —O—, —S—, —C(O)—, —N(R²⁰)C(O)—, —C(O)N(R²¹)—, —N(R²⁰)C(O)N(R²¹)—, —C(O)O—, —OC(O)—, —N(R²⁰)C(O)O—, —OC(O)N(R²¹)—, —OPO₂—O—, —O—P(O)(S)—O—, —O—P(O)(R²²)—O—, —O—P(S)(R²²)—O—, —O—P(O)(NR²⁰R²¹)—N—, —O—P(S)(NR²⁰R²¹)—N—, —O—P(O)(NR²⁰R²¹)—O—, —O—P(S)(NR²⁰R²¹)—O—, —P(O)(NR²⁰R²¹)—N—, —P(S)(NR²⁰R²¹)—N—, —P(O)(NR²⁰R²¹)—, —P(S)(NR²⁰R²¹)—O—, —S—S—, substituted (e.g., substituted with a substituent group, a size-limited substituent group, or lower substituent group) alkylene (e.g., C₁-C₂₀, C₁-C₁₂, C₁-C₈, C₁-C₆, C₁-C₄, or C₁-C₂), substituted (e.g., substituted with a substituent group, a size-limited substituent group, or lower substituent group) heteroalkylene (e.g., 2 to 20 membered, 2 to 12 membered, 2 to 8 membered, 2 to 6 membered, 4 to 6 membered, 2 to 3 membered, or 4 to 5 membered), substituted (e.g., substituted with a substituent group, a size-limited substituent group, or lower substituent group) cycloalkylene (e.g., C₃-C₁₀, C₃-C₈, C₃-C₆, C₄-C₆, or C₅-C₆), substituted (e.g., substituted with a substituent group, a size-limited substituent group, or lower substituent group) heterocycloalkylene (e.g., 3 to 10 membered, 3 to 8 membered, 3 to 6 membered, 4 to 6 membered, 4 to 5 membered, or 5 to 6 membered), substituted (e.g., substituted with a substituent group, a size-limited substituent group, or lower substituent group) arylene (e.g., C₆-C₁₂, C₆-C₁₀, or phenyl), or substituted (e.g., substituted with a substituent group, a size-limited substituent group, or lower substituent group) heteroarylene (e.g., 5 to 12 membered, 5 to 10 membered, 5 to 9 membered, or 5 to 6 membered).

In embodiments, L^{1A} is independently a bond, —N(R²⁰)—, —O—, —S—, —C(O)—, —N(R²⁰)C(O)—, —C(O)N(R²¹)—, —N(R²⁰)C(O)N(R²¹)—, —C(O)O—, —OC(O)—, —N(R²⁰)C(O)O—, —OC(O)N(R²¹)—, —OPO₂—O—, —O—P(O)(S)—O—, —O—P(O)(R²²)—O—, —O—P(S)(R²²)—O—, —O—P(O)(NR²⁰R²¹)—N—, —O—P(S)(NR²⁰R²¹)—N—, —O—P(O)(NR²⁰R²¹)—O—, —O—P(S)(NR²⁰R²¹)—O—, —P(O)(NR²⁰R²¹)—N—, —P(S)(NR²⁰R²¹)—N—, —P(O)(NR²⁰R²¹)—, —P(S)(NR²⁰R²¹)—O—, —S—S—, unsubstituted heteroalkylene (e.g., 2 to 20 membered, 2 to 12 membered, 2 to 8 membered, 2 to 6 membered, 4 to 6 membered, 2 to 3 membered, or 4 to 5 membered), unsubstituted cycloalkylene (e.g., C₃-C₁₀, C₃-C₈, C₃-C₆, C₄-C₆, or C₅-C₆), unsubstituted heterocycloalkylene (e.g., 3 to 10 membered, 3 to 8 membered, 3 to 6 membered, 4 to 6 membered, 4 to 5 membered, or 5 to 6 membered), unsubstituted arylene (e.g., C₆-C₁₂, C₆-C₁₀, or phenyl), or unsubstituted heteroarylene (e.g., 5 to 12 membered, 5 to 10 membered, 5 to 9 membered, or 5 to 6 membered). In embodiments, when L^{1A} is substituted, L^{1A} is substituted with a substituent group. In embodiments, when L^{1A} is substituted, L^{1A} is substituted with a size-limited substituent group. In embodiments, when L^{1A} is substituted, L^{1A} is substituted with a lower substituent group.

L^{1B} is independently a bond, —N(R²⁰)—, —O—, —S—, —C(O)—, —N(R²⁰)C(O)—, —C(O)N(R²¹)—, —N(R²⁰)C(O)N(R²¹)—, —C(O)O—, —OC(O)—, —N(R²⁰)C(O)O—, —OC(O)N(R²¹)—, —OPO₂—O—, —O—P(O)(S)—O—, —O—P(O)(R²²)—O—, —O—P(S)(R²²)—O—, —O—P(O)(NR²⁰R²¹)—N—, —O—P(S)(NR²⁰R²¹)—N—, —O—P(O)(NR²⁰R²¹)—O—, —O—P(S)(NR²⁰R²¹)—O—, —P(O)(NR²⁰R²¹)—N—, —P(S)(NR²⁰R²¹)—N—, —P(O)(NR²⁰R²¹)—O—, —P(S)(NR²⁰R²¹)—O—, —S—S—, substituted (e.g., substituted with a substituent group, a size-limited substituent group, or lower substituent group) or unsubstituted alkylene (e.g., C₁-C₂₀, C₁-C₁₂, C₁-C₈, C₁-C₆, C₁-C₄, or C₁-C₂), substituted (e.g., substituted with a substituent group, a size-limited substituent group, or lower substituent group) or unsubstituted heteroalkylene (e.g., 2 to 20 membered, 2 to 12 membered, 2 to 8 membered, 2 to 6 membered, 4 to 6 membered, 2 to 3 membered, or 4 to 5 membered), substituted (e.g., substituted with a substituent group, a size-limited substituent group, or lower substituent group) or unsubstituted cycloalkylene (e.g., C₃-C₁₀, C₃-C₈, C₃-C₆, C₄-C₆, or C₅-C₆), substituted (e.g., substituted with a substituent group, a size-limited substituent group, or lower substituent group) or unsubstituted heterocycloalkylene (e.g., 3 to 10 membered, 3 to 8 membered, 3 to 6 membered, 4 to 6 membered, 4 to 5 membered, or 5 to 6 membered), substituted (e.g., substituted with a substituent group, a size-limited substituent group, or lower substituent group) or unsubstituted arylene (e.g., C₆-C₁₂, C₆-C₁₀, or phenyl), or substituted (e.g., substituted with a substituent group, a size-limited substituent group, or lower substituent group) or unsubstituted heteroarylene (e.g., 5 to 12 membered, 5 to 10 membered, 5 to 9 membered, or 5 to 6 membered). In embodiments, L^{1B} is independently a bond, —N(R²⁰)—, —O—, —S—, —C(O)—, —N(R²⁰)C(O)—, —C(O)N(R²¹)—, —N(R²⁰)C(O)N(R²¹)—, —C(O)O—, —OC(O)—, —N(R²⁰)C(O)O—, —OC(O)N(R²¹)—, —OPO₂—O—, —O—P(O)(S)—O—, —O—P(O)(R²²)—O—, —O—P(S)(R²²)—O—, —O—P(O)(NR²⁰R²¹)—N—, —O—P(S)(NR²⁰R²¹)—N—, —O—P(O)(NR²⁰R²¹)—O—, —O—P(S)(NR²⁰R²¹)—O—, —P(O)(NR²⁰R²¹)—N—, —P(S)(NR²⁰R²¹)—N—, —P(O)(NR²⁰R²¹)—, —P(S)(NR²⁰R²¹)—O—, —S—S—, substituted (e.g., substituted with a substituent group, a size-limited substituent group, or lower substituent group) alkylene (e.g., C₁-C₂₀, C₁-C₁₂, C₁-C₈, C₁-C₆, C₁-C₄, or C₁-C₂), substituted (e.g., substituted with a substituent group, a size-limited substituent group, or lower substituent group) heteroalkylene (e.g., 2 to 20 membered, 2 to 12 membered, 2 to 8 membered, 2 to 6 membered, 4 to 6 membered, 2 to 3 membered, or 4 to 5 membered), substituted (e.g., substituted with a substituent group, a size-limited substituent group, or lower substituent group) cycloalkylene (e.g., C₃-C₁₀, C₃-C₈, C₃-C₆, C₄-C₆, or C₅-C₆), substituted (e.g., substituted with a substituent group, a size-limited substituent group, or lower substituent group) heterocycloalkylene (e.g., 3 to 10 membered, 3 to 8 membered, 3 to 6 membered, 4 to 6 membered, 4 to 5 membered, or 5 to 6 membered), substituted (e.g., substituted with a substituent group, a size-limited substituent group, or lower substituent group) arylene (e.g., C₆-C₁₂, C₆-C₁₀, or phenyl), or substituted (e.g., substituted with a substituent group, a size-limited substituent group, or lower substituent group) heteroarylene (e.g., 5 to 12 membered, 5 to 10 membered, 5 to 9 membered, or 5 to 6 membered).

In embodiments, L^{1B} is independently a bond, —N(R²⁰)—, —O—, —S—, —C(O)—, —N(R²⁰)C(O)—, —C(O)N(R²¹)—, —N(R²⁰)C(O)N(R²¹)—, —C(O)O—, —OC(O)—, —N(R²⁰)C(O)O—, —OC(O)N(R²¹)—, —OPO₂—O—, —O—P(O)(S)—O—, —O—P(O)(R²²)—O—, —O—P(S)(R²²)—O—, —O—P(O)(NR²⁰R²¹)—N—, —O—P(S)(NR²⁰R²¹)—N—, —O—P(O)(NR²⁰R²¹)—O—, —O—P(S)(NR²⁰R²¹)—O—, —P(O)(NR²⁰R²¹)—N—, —P(S)(NR²⁰R²¹)—N—, —P(O)(NR²⁰R²¹)—O—, —P(S)(NR²⁰R²¹)—O—, —S—S—, unsubstituted alkylene (e.g., C₁-C₂₀, C₁-C₁₂, C₁-C₈, C₁-C₆, C₁-C₄, or C₁-C₂), unsubstituted heteroalkylene (e.g., 2 to 20 membered, 2 to 12 membered, 2 to 8 membered, 2 to 6 membered, 4 to 6 membered, 2 to 3 membered, or 4 to 5 membered), unsubstituted cycloalkylene (e.g., C₃-C₁₀, C₃-C₈, C₃-C₆, C₄-C₆, or C₅-C₆), unsubstituted heterocycloalkylene (e.g., 3 to 10 membered, 3 to 8 membered, 3 to 6 membered, 4 to 6 membered, 4 to 5 membered, or 5 to 6 membered), unsubstituted arylene (e.g., C₆-C₁₂, C₆-C₁₀, or phenyl), or unsubstituted heteroarylene (e.g., 5 to 12 membered, 5 to 10 membered, 5 to 9 membered, or 5 to 6 membered). In embodiments, when L^{1B} is substituted, L^{1B} is substituted with a substituent group. In embodiments, when L^{1B} is substituted, L^{1B} is substituted with a size-limited substituent group. In embodiments, when L^{1B} is substituted, L^{1B} is substituted with a lower substituent group.

L^{1C} is independently a bond, —N(R²⁰)—, —O—, —S—, —C(O)—, —N(R²⁰)C(O)—, —C(O)N(R²¹)—, —N(R²⁰)C(O)N(R²¹)—, —C(O)O—, —OC(O)—, —N(R²⁰)C(O)O—, —OC(O)N(R²¹)—, —OPO₂—O—, —O—P(O)(S)—O—, —O—P(O)(R²²)—O—, —O—P(S)(R²²)—O—, —O—P(O)(NR²⁰R²¹)—N—, —O—P(S)(NR²⁰R²¹)—N—, —O—P(O)(NR²⁰R²¹)—O—, —O—P(S)(NR²⁰R²¹)—O—, —P(O)(NR²⁰R²¹)—N—, —P(S)(NR²⁰R²¹)—N—, —P(O)(NR²⁰R²¹)—O—, —P(S)(NR²⁰R²¹)—O—, —S—S—, substituted (e.g., substituted with a substituent group, a size-limited substituent group, or lower substituent group) or unsubstituted alkylene (e.g., C₁-C₂₀, C₁-C₁₂, C₁-C₈, C₁-C₆, C₁-C₄, or C₁-C₂), substituted (e.g., substituted with a substituent group, a size-limited substituent group, or lower substituent group) or unsubstituted heteroalkylene (e.g., 2 to 20 membered, 2 to 12 membered, 2 to 8 membered, 2 to 6 membered, 4 to 6 membered, 2 to 3 membered, or 4 to 5 membered), substituted (e.g., substituted with a substituent group, a size-limited substituent group, or lower substituent group) or unsubstituted cycloalkylene (e.g., C₃-C₁₀, C₃-C₈, C₃-C₆, C₄-C₆, or C₅-C₆), substituted (e.g., substituted with a substituent group, a size-limited substituent group, or lower substituent group) or unsubstituted heterocycloalkylene (e.g., 3 to 10 membered, 3 to 8 membered, 3 to 6 membered, 4 to 6 membered, 4 to 5 membered, or 5 to 6 membered), substituted (e.g., substituted with a substituent group, a size-limited substituent group, or lower substituent group) or unsubstituted arylene (e.g., C₆-C₁₂, C₆-C₁₀, or phenyl), or substituted (e.g., substituted with a substituent group, a size-limited substituent group, or lower substituent group) or unsubstituted heteroarylene (e.g., 5 to 12 membered, 5 to 10 membered, 5 to 9 membered, or 5 to 6 membered). In embodiments, L^{1C} is independently a bond, —N(R²⁰)—, —O—, —S—, —C(O)—, —N(R²⁰)C(O)—, —C(O)N(R²¹)—, —N(R²⁰)C(O)N(R²¹)—, —C(O)O—, —OC(O)—, —N(R²⁰)C(O)O—, —OC(O)N(R²¹)—, —OPO₂—O—, —O—P(O)(S)—O—, —O—P(O)(R²²)—O—, —O—P(S)(R²²)—O—, —O—P(O)(NR²⁰R²¹)—N—, —O—P(S)(NR²⁰R²¹)—N—, —O—P(O)(NR²⁰R²¹)—O—, —O—P(S)(NR²⁰R²¹)—O—, —P(O)(NR²⁰R²¹)—N—, —P(S)(NR²⁰R²¹)—N—, —P(O)(NR²⁰R²¹)—O—, —P(S)(NR²⁰R²¹)—O—, —S—S—, substituted (e.g., substituted with a substituent group, a size-limited substituent group, or lower substituent group) alkylene (e.g., C₁-C₂₀, C₁-C₁₂, C₁-C₈, C₁-C₆, C₁-C₄, or C₁-C₂), substituted (e.g., substituted with a substituent group, a size-limited substituent group, or lower substituent group) heteroalkylene (e.g., 2 to 20 membered, 2 to 12 membered, 2 to 8 membered, 2 to 6 membered, 4 to 6 membered, 2 to 3 membered, or 4 to 5 membered), substituted (e.g., substituted with a substituent group, a size-limited substituent group, or lower substituent group) cycloalkylene (e.g., C₃-C₁₀, C₃-C₈, C₃-C₆, C₄-C₆, or C₅-C₆), substituted (e.g., substituted with a substituent group, a size-limited substituent group, or lower substituent group) heterocycloalkylene (e.g., 3 to 10 membered, 3 to 8 membered, 3 to 6 membered, 4 to 6 membered, 4 to 5 membered, or 5 to 6 membered), substituted (e.g., substituted with a substituent group, a size-limited substituent group, or lower substituent group) arylene (e.g., C₆-C₁₂, C₆-C₁₀, or phenyl), or substituted (e.g., substituted with a substituent group, a size-limited substituent group, or lower substituent group) heteroarylene (e.g., 5 to 12 membered, 5 to 10 membered, 5 to 9 membered, or 5 to 6 membered).

In embodiments, L^{1C} is independently a bond, N(R²⁰)—, —O—, —S—, —C(O)—, —N(R²⁰)C(O)—, —C(O)N(R²¹)—, —N(R²⁰)C(O)N(R²¹)—, —C(O)O—, —OC(O)—, —N(R²⁰)C(O)O—, —OC(O)N(R²¹)—, —OPO₂—O—, —O—P(O)(S)—O—, —O—P(O)(R²²)—O—, —O—P(S)(R²²)—O—, —O—P(O)(NR²⁰R²¹)—N—, —O—P(S)(NR²⁰R²¹)—N—, —O—P(O)(NR²⁰R²¹)—O—, —O—P(S)(NR²⁰R²¹)—O—, —P(O)(NR²⁰R²¹)—N—, —P(S)(NR²⁰R²¹)—N—, —P(O)(NR²⁰R²¹)—O—, —P(S)(NR²⁰R²¹)—O—, —S—S—, unsubstituted alkylene (e.g., C₁-C₂₀, C₁-C₁₂, C₁-C₈, C₁-C₆, C₁-C₄, or C₁-C₂), unsubstituted heteroalkylene (e.g., 2 to 20 membered, 2 to 12 membered, 2 to 8 membered, 2 to 6 membered, 4 to 6 membered, 2 to 3 membered, or 4 to 5 membered), unsubstituted cycloalkylene (e.g., C₃-C₁₀, C₃-C₈, C₃-C₆, C₄-C₆, or C₅-C₆), unsubstituted heterocycloalkylene (e.g., 3 to 10 membered, 3 to 8 membered, 3 to 6 membered, 4 to 6 membered, 4 to 5 membered, or 5 to 6 membered), unsubstituted arylene (e.g., C₆-C₁₂, C₆-C₁₀, or phenyl), or unsubstituted heteroarylene (e.g., 5 to 12 membered, 5 to 10 membered, 5 to 9 membered, or 5 to 6 membered). In embodiments, when L^{1C} is substituted, L^{1C} is substituted with a substituent group. In embodiments, when L^{1C} is substituted, L^{1C} is substituted with a size-limited substituent group. In embodiments, when L^{1C} is substituted, L^{1C} is substituted with a lower substituent group.

R^{1C} is independently substituted (e.g., substituted with a substituent group, a size-limited substituent group, or lower substituent group) or unsubstituted alkyl (e.g., C₁-C₂₀, C₁-C₁₂, C₁-C₈, C₁-C₆, C₁-C₄, or C₁-C₂), substituted (e.g., substituted with a substituent group, a size-limited substituent group, or lower substituent group) or unsubstituted heteroalkyl (e.g., 2 to 20 membered, 2 to 12 membered, 2 to 8 membered, 2 to 6 membered, 4 to 6 membered, 2 to 3 membered, or 4 to 5 membered), substituted (e.g., substituted with a substituent group, a size-limited substituent group, or lower substituent group) or unsubstituted cycloalkyl (e.g., C₃-C₁₀, C₃-C₈, C₃-C₆, C₄-C₆, or C₅-C₆), substituted (e.g., substituted with a substituent group, a size-limited substituent group, or lower substituent group) or unsubstituted heterocycloalkyl (e.g., 3 to 10 membered, 3 to 8 membered, 3 to 6 membered, 4 to 6 membered, 4 to 5 membered, or 5 to 6 membered), substituted (e.g., substituted with a substituent group, a size-limited substituent group, or lower substituent group) or unsubstituted aryl (e.g., C₆-C₁₂, C₆-C₁₀, or phenyl), or substituted (e.g., substituted with a substituent group, a size-limited substituent group, or lower substituent group) or unsubstituted heteroaryl (e.g., 5 to 12 membered, 5 to 10 membered, 5 to 9 membered, or 5 to 6 membered). In embodiments, R^{1C} is independently substituted (e.g., substituted with a substituent group, a size-limited substituent group, or lower substituent group) alkyl (e.g., C₁-C₂₀, C₁-C₁₂, C₁-C₈, C₁-C₆, C₁-C₄, or C₁-C₂), substituted (e.g., substituted with a substituent group, a size-limited substituent group, or lower substituent group) heteroalkyl (e.g., 2 to 20 membered, 2 to 12 membered, 2 to 8 membered, 2 to 6 membered, 4 to 6 membered, 2 to 3 membered, or 4 to 5 membered), substituted (e.g., substituted with a substituent group, a size-limited substituent group, or lower substituent group) cycloalkyl (e.g., C₃-C₁₀, C₃-C₈, C₃-C₆, C₄-C₆, or C₅-C₆), substituted (e.g., substituted with a substituent group, a size-limited substituent group, or lower substituent group) heterocycloalkyl (e.g., 3 to 10 membered, 3 to 8 membered, 3 to 6 membered, 4 to 6 membered, 4 to 5 membered, or 5 to 6 membered), substituted (e.g., substituted with a substituent group, a size-limited substituent group, or lower substituent group) aryl (e.g., C₆-C₁₂, C₆-C₁₀, or phenyl), or substituted (e.g., substituted with a substituent group, a size-limited substituent group, or lower substituent group) heteroaryl (e.g., 5 to 12 membered, 5 to 10 membered, 5 to 9 membered, or 5 to 6 membered). In embodiments, R^{1C} is independently unsubstituted alkyl (e.g., C₁-C₂₀, C₁-C₁₂, C₁-C₈, C₁-C₆, C₁-C₄, or C₁-C₂), unsubstituted heteroalkyl (e.g., 2 to 20 membered, 2 to 12 membered, 2 to 8 membered, 2 to 6 membered, 4 to 6 membered, 2 to 3 membered, or 4 to 5 membered), unsubstituted cycloalkyl (e.g., C₃-C₁₀, C₃-C₈, C₃-C₆, C₄-C₆, or C₅-C₆), unsubstituted heterocycloalkyl (e.g., 3 to 10 membered, 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). In embodiments, when R^{1C} is substituted, R^{1C} is substituted with a substituent group. In embodiments, when R^{1C} is substituted, R^{1C} is substituted with a size-limited substituent group. In embodiments, when R^{1C} is substituted, R^{1C} is substituted with a lower substituent group. In embodiments, R^{1C} is substituted with oxo (═O).

L^{1D} is independently a bond, —N(R²⁰)—, —O—, —S—, —C(O)—, —N(R²⁰)C(O)—, —C(O)N(R²¹)—, —N(R²⁰)C(O)N(R²¹)—, —C(O)O—, —OC(O)—, —N(R²⁰)C(O)O—, —OC(O)N(R²¹)—, —OPO₂—O—, —O—P(O)(S)—O—, —O—P(O)(R²²)—O—, —O—P(S)(R²²)—O—, —O—P(O)(NR²⁰R²¹)—N—, —O—P(S)(NR²⁰R²¹)—N—, —O—P(O)(NR²⁰R²¹)—O—, —O—P(S)(NR²⁰R²¹)—O—, —P(O)(NR²⁰R²¹)—N—, —P(S)(NR²⁰R²¹)—N—, —P(O)(NR²⁰R²¹)—O—, —P(S)(NR²⁰R²¹)—O—, —S—S—, substituted (e.g., substituted with a substituent group, a size-limited substituent group, or lower substituent group) or unsubstituted alkylene (e.g., C₁-C₂₀, C₁-C₁₂, C₁-C₈, C₁-C₆, C₁-C₄, or C₁-C₂), substituted (e.g., substituted with a substituent group, a size-limited substituent group, or lower substituent group) or unsubstituted heteroalkylene (e.g., 2 to 20 membered, 2 to 12 membered, 2 to 8 membered, 2 to 6 membered, 4 to 6 membered, 2 to 3 membered, or 4 to 5 membered), substituted (e.g., substituted with a substituent group, a size-limited substituent group, or lower substituent group) or unsubstituted cycloalkylene (e.g., C₃-C₁₀, C₃-C₈, C₃-C₆, C₄-C₆, or C₅-C₆), substituted (e.g., substituted with a substituent group, a size-limited substituent group, or lower substituent group) or unsubstituted heterocycloalkylene (e.g., 3 to 10 membered, 3 to 8 membered, 3 to 6 membered, 4 to 6 membered, 4 to 5 membered, or 5 to 6 membered), substituted (e.g., substituted with a substituent group, a size-limited substituent group, or lower substituent group) or unsubstituted arylene (e.g., C₆-C₁₂, C₆-C₁₀, or phenyl), or substituted (e.g., substituted with a substituent group, a size-limited substituent group, or lower substituent group) or unsubstituted heteroarylene (e.g., 5 to 12 membered, 5 to 10 membered, 5 to 9 membered, or 5 to 6 membered). In embodiments, L^{1D} is independently a bond, —N(R²⁰)—, —O—, —S—, —C(O)—, —N(R²⁰)C(O)—, —C(O)N(R²¹)—, —(R²⁰)C(O)N(R²¹)—, —C(O)O—, —OC(O)—, —N(R²⁰)C(O)O—, —OC(O)N(R²¹)—, —OPO₂—O—, —O—P(O)(S)—O—, —O—P(O)(R²²)—O—, —O—P(S)(R²²)—O—, —O—P(O)(NR²⁰R²¹)—N—, —O—P(S)(NR²⁰R²¹)—N—, —O—P(O)(NR²⁰R²¹)—O—, —O—P(S)(NR²⁰R²¹)—O—, —P(O)(NR²⁰R²¹)—N—, —P(S)(NR²⁰R²¹)—N—, —P(O)(NR²⁰R²¹)—O—, —P(S)(NR²⁰R²¹)—O—, —S—S—, substituted (e.g., substituted with a substituent group, a size-limited substituent group, or lower substituent group) alkylene (e.g., C₁-C₂₀, C₁-C₁₂, C₁-C₈, C₁-C₆, C₁-C₄, or C₁-C₂), substituted (e.g., substituted with a substituent group, a size-limited substituent group, or lower substituent group) heteroalkylene (e.g., 2 to 20 membered, 2 to 12 membered, 2 to 8 membered, 2 to 6 membered, 4 to 6 membered, 2 to 3 membered, or 4 to 5 membered), substituted (e.g., substituted with a substituent group, a size-limited substituent group, or lower substituent group) cycloalkylene (e.g., C₃-C₁₀, C₃-C₈, C₃-C₆, C₄-C₆, or C₅-C₆), substituted (e.g., substituted with a substituent group, a size-limited substituent group, or lower substituent group) heterocycloalkylene (e.g., 3 to 10 membered, 3 to 8 membered, 3 to 6 membered, 4 to 6 membered, 4 to 5 membered, or 5 to 6 membered), substituted (e.g., substituted with a substituent group, a size-limited substituent group, or lower substituent group) arylene (e.g., C₆-C₁₂, C₆-C₁₀, or phenyl), or substituted (e.g., substituted with a substituent group, a size-limited substituent group, or lower substituent group) heteroarylene (e.g., 5 to 12 membered, 5 to 10 membered, 5 to 9 membered, or 5 to 6 membered).

In embodiments, L^{ID} is independently a bond, —N(R²⁰)—, —O—, —S—, —C(O)—, —N(R²⁰)C(O)—, —C(O)N(R²¹)—, —N(R²⁰)C(O)N(R²¹)—, —C(O)O—, —OC(O)—, —N(R²⁰)C(O)O—, —OC(O)N(R²¹)—, —OPO₂—O—, —O—P(O)(S)—O—, —O—P(O)(R²²)—O—, —O—P(S)(R²²)—O—, —O—P(O)(NR²⁰R²¹)—N—, —O—P(S)(NR²⁰R²¹)—N—, —O—P(O)(NR²⁰R²¹)—O—, —O—P(S)(NR²⁰R²¹)—O—, —P(O)(NR²⁰R²¹)—N—, —P(S)(NR²⁰R²¹)—N—, —P(O)(NR²⁰R²¹)—O—, —P(S)(NR²⁰R²¹)—O—, —S—S—, unsubstituted alkylene (e.g., C₁-C₂₀, C₁-C₁₂, C₁-C₈, C₁-C₆, C₁-C₄, or C₁-C₂), unsubstituted heteroalkylene (e.g., 2 to 20 membered, 2 to 12 membered, 2 to 8 membered, 2 to 6 membered, 4 to 6 membered, 2 to 3 membered, or 4 to 5 membered), unsubstituted cycloalkylene (e.g., C₃-C₁₀, C₃-C₈, C₃-C₆, C₄-C₆, or C₅-C₆), unsubstituted heterocycloalkylene (e.g., 3 to 10 membered, 3 to 8 membered, 3 to 6 membered, 4 to 6 membered, 4 to 5 membered, or 5 to 6 membered), unsubstituted arylene (e.g., C₆-C₁₂, C₆-C₁₀, or phenyl), or unsubstituted heteroarylene (e.g., 5 to 12 membered, 5 to 10 membered, 5 to 9 membered, or 5 to 6 membered). In embodiments, when L^{1D} is substituted, L^{1D} is substituted with a substituent group. In embodiments, when L^{1D} is substituted, L^{1D} is substituted with a size-limited substituent group. In embodiments, when L^{1D} is substituted, L^{1D} is substituted with a lower substituent group.

R^{1D} is independently substituted (e.g., substituted with a substituent group, a size-limited substituent group, or lower substituent group) or unsubstituted alkyl (e.g., C₁-C₂₀, C₁-C₁₂, C₁-C₈, C₁-C₆, C₁-C₄, or C₁-C₂), substituted (e.g., substituted with a substituent group, a size-limited substituent group, or lower substituent group) or unsubstituted heteroalkyl (e.g., 2 to 20 membered, 2 to 12 membered, 2 to 8 membered, 2 to 6 membered, 4 to 6 membered, 2 to 3 membered, or 4 to 5 membered), substituted (e.g., substituted with a substituent group, a size-limited substituent group, or lower substituent group) or unsubstituted cycloalkyl (e.g., C₃-C₁₀, C₃-C₈, C₃-C₆, C₄-C₆, or C₅-C₆), substituted (e.g., substituted with a substituent group, a size-limited substituent group, or lower substituent group) or unsubstituted heterocycloalkyl (e.g., 3 to 10 membered, 3 to 8 membered, 3 to 6 membered, 4 to 6 membered, 4 to 5 membered, or 5 to 6 membered), substituted (e.g., substituted with a substituent group, a size-limited substituent group, or lower substituent group) or unsubstituted aryl (e.g., C₆-C₁₂, C₆-C₁₀, or phenyl), or substituted (e.g., substituted with a substituent group, a size-limited substituent group, or lower substituent group) or unsubstituted heteroaryl (e.g., 5 to 12 membered, 5 to 10 membered, 5 to 9 membered, or 5 to 6 membered). In embodiments, R^{1D} is independently substituted (e.g., substituted with a substituent group, a size-limited substituent group, or lower substituent group) alkyl (e.g., C₁-C₂₀, C₁-C₁₂, C₁-C₈, C₁-C₆, C₁-C₄, or C₁-C₂), substituted (e.g., substituted with a substituent group, a size-limited substituent group, or lower substituent group) heteroalkyl (e.g., 2 to 20 membered, 2 to 12 membered, 2 to 8 membered, 2 to 6 membered, 4 to 6 membered, 2 to 3 membered, or 4 to 5 membered), substituted (e.g., substituted with a substituent group, a size-limited substituent group, or lower substituent group) cycloalkyl (e.g., C₃-C₁₀, C₃-C₈, C₃-C₆, C₄-C₆, or C₅-C₆), substituted (e.g., substituted with a substituent group, a size-limited substituent group, or lower substituent group) heterocycloalkyl (e.g., 3 to 10 membered, 3 to 8 membered, 3 to 6 membered, 4 to 6 membered, 4 to 5 membered, or 5 to 6 membered), substituted (e.g., substituted with a substituent group, a size-limited substituent group, or lower substituent group) aryl (e.g., C₆-C₁₂, C₆-C₁₀, or phenyl), or substituted (e.g., substituted with a substituent group, a size-limited substituent group, or lower substituent group) heteroaryl (e.g., 5 to 12 membered, 5 to 10 membered, 5 to 9 membered, or 5 to 6 membered). In embodiments, R^{1D} is independently unsubstituted alkyl (e.g., C₁-C₂₀, C₁-C₁₂, C₁-C₈, C₁-C₆, C₁-C₄, or C₁-C₂), unsubstituted heteroalkyl (e.g., 2 to 20 membered, 2 to 12 membered, 2 to 8 membered, 2 to 6 membered, 4 to 6 membered, 2 to 3 membered, or 4 to 5 membered), unsubstituted cycloalkyl (e.g., C₃-C₁₀, C₃-C₈, C₃-C₆, C₄-C₆, or C₅-C₆), unsubstituted heterocycloalkyl (e.g., 3 to 10 membered, 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). In embodiments, when R^{1D} is substituted, R^{1D} is substituted with a substituent group. In embodiments, when R^{1D} is substituted, R^{1D} is substituted with a size-limited substituent group. In embodiments, when R^{1D} is substituted, R^{1D} is substituted with a lower substituent group.

L^{1E} is independently a bond, —N(R²⁰)—, —O—, —S—, —C(O)—, —N(R²⁰)C(O)—, —C(O)N(R²¹)—, —N(R²⁰)C(O)N(R²¹)—, —C(O)O—, —OC(O)—, —N(R²⁰)C(O)O—, —OC(O)N(R²¹)—, —OPO₂—O—, —O—P(O)(S)—O—, —O—P(O)(R²²)—O—, —O—P(S)(R²²)—O—, —O—P(O)(NR²⁰R²¹)—N—, —O—P(S)(NR²⁰R²¹)—N—, —O—P(O)(NR²⁰R²¹)—O—, —O—P(S)(NR²⁰R²¹)—O—, —P(O)(NR²⁰R²¹)—N—, —P(S)(NR²⁰R²¹)—N—, —P(O)(NR²⁰R²¹)—O—, —P(S)(NR²⁰R²¹)—O—, —S—S—, substituted (e.g., substituted with a substituent group, a size-limited substituent group, or lower substituent group) or unsubstituted alkylene (e.g., C₁-C₂₀, C₁-C₁₂, C₁-C₈, C₁-C₆, C₁-C₄, or C₁-C₂), substituted (e.g., substituted with a substituent group, a size-limited substituent group, or lower substituent group) or unsubstituted heteroalkylene (e.g., 2 to 20 membered, 2 to 12 membered, 2 to 8 membered, 2 to 6 membered, 4 to 6 membered, 2 to 3 membered, or 4 to 5 membered), substituted (e.g., substituted with a substituent group, a size-limited substituent group, or lower substituent group) or unsubstituted cycloalkylene (e.g., C₃-C₁₀, C₃-C₈, C₃-C₆, C₄-C₆, or C₅-C₆), substituted (e.g., substituted with a substituent group, a size-limited substituent group, or lower substituent group) or unsubstituted heterocycloalkylene (e.g., 3 to 10 membered, 3 to 8 membered, 3 to 6 membered, 4 to 6 membered, 4 to 5 membered, or 5 to 6 membered), substituted (e.g., substituted with a substituent group, a size-limited substituent group, or lower substituent group) or unsubstituted arylene (e.g., C₆-C₁₂, C₆-C₁₀, or phenyl), or substituted (e.g., substituted with a substituent group, a size-limited substituent group, or lower substituent group) or unsubstituted heteroarylene (e.g., 5 to 12 membered, 5 to 10 membered, 5 to 9 membered, or 5 to 6 membered). In embodiments, L^{1E} is independently a bond, —N(R²⁰)—, —O—, —S—, C(O)—, —N(R²⁰)C(O)—, —C(O)N(R²¹)—, —N(R²⁰)C(O)N(R²¹)—, —C(O)O—, —OC(O)—, —N(R²⁰)C(O)O—, —OC(O)N(R²¹)—, —OPO₂—O—, —O—P(O)(S)—O—, —O—P(O)(R²²)—O—, —O—P(S)(R²²)—O—, —O—P(O)(NR²⁰R²¹)—N—, —O—P(S)(NR²⁰R²¹)—N—, —O—P(O)(NR²⁰R²¹)—O—, —O—P(S)(NR²⁰R²¹)—O—, —P(O)(NR²⁰R²¹)—N—, —P(S)(NR²⁰R²¹)—N—, —P(O)(NR²⁰R²¹)—, —P(S)(NR²⁰R²¹)—O—, —S—S—, substituted (e.g., substituted with a substituent group, a size-limited substituent group, or lower substituent group) alkylene (e.g., C₁-C₂₀, C₁-C₁₂, C₁-C₈, C₁-C₆, C₁-C₄, or C₁-C₂), substituted (e.g., substituted with a substituent group, a size-limited substituent group, or lower substituent group) heteroalkylene (e.g., 2 to 20 membered, 2 to 12 membered, 2 to 8 membered, 2 to 6 membered, 4 to 6 membered, 2 to 3 membered, or 4 to 5 membered), substituted (e.g., substituted with a substituent group, a size-limited substituent group, or lower substituent group) cycloalkylene (e.g., C₃-C₁₀, C₃-C₈, C₃-C₆, C₄-C₆, or C₅-C₆), substituted (e.g., substituted with a substituent group, a size-limited substituent group, or lower substituent group) heterocycloalkylene (e.g., 3 to 10 membered, 3 to 8 membered, 3 to 6 membered, 4 to 6 membered, 4 to 5 membered, or 5 to 6 membered), substituted (e.g., substituted with a substituent group, a size-limited substituent group, or lower substituent group) arylene (e.g., C₆-C₁₂, C₆-C₁₀, or phenyl), or substituted (e.g., substituted with a substituent group, a size-limited substituent group, or lower substituent group) heteroarylene (e.g., 5 to 12 membered, 5 to 10 membered, 5 to 9 membered, or 5 to 6 membered).

In embodiments, L^{1E} is independently a bond, —N(R²⁰)—, —O—, —S—, —C(O)—, —N(R²⁰)C(O)—, —C(O)N(R²¹)—, —N(R²⁰)C(O)N(R²¹)—, —C(O)O—, —OC(O)—, —N(R²⁰)C(O)O—, —OC(O)N(R²¹)—, —OPO₂—O—, —O—P(O)(S)—O—, —O—P(O)(R²²)—O—, —O—P(S)(R²²)—O—, —O—P(O)(NR²⁰R²¹)—N—, —O—P(S)(NR²⁰R²¹)—N—, —O—P(O)(NR²⁰R²¹)—O—, —O—P(S)(NR²⁰R²¹)—O—, —P(O)(NR²⁰R²¹)—N—, —P(S)(NR²⁰R²¹)—N—, —P(O)(NR²⁰R²¹)—O—, —P(S)(NR²⁰R²¹)—O—, —S—S—, unsubstituted heteroalkylene (e.g., 2 to 20 membered, 2 to 12 membered, 2 to 8 membered, 2 to 6 membered, 4 to 6 membered, 2 to 3 membered, or 4 to 5 membered), unsubstituted cycloalkylene (e.g., C₃-C₁₀, C₃-C₈, C₃-C₆, C₄-C₆, or C₅-C₆), unsubstituted heterocycloalkylene (e.g., 3 to 10 membered, 3 to 8 membered, 3 to 6 membered, 4 to 6 membered, 4 to 5 membered, or 5 to 6 membered), unsubstituted arylene (e.g., C₆-C₁₂, C₆-C₁₀, or phenyl), or unsubstituted heteroarylene (e.g., 5 to 12 membered, 5 to 10 membered, 5 to 9 membered, or 5 to 6 membered). In embodiments, when L^{1E} is substituted, L¹E is substituted with a substituent group. In embodiments, when L^{1E} is substituted, L^{1E} is substituted with a size-limited substituent group. In embodiments, when L^{1E} is substituted, L^{1E} is substituted with a lower substituent group.

R^{1E} is independently substituted (e.g., substituted with a substituent group, a size-limited substituent group, or lower substituent group) or unsubstituted alkyl (e.g., C₁-C₂₀, C₁-C₁₂, C₁-C₈, C₁-C₆, C₁-C₄, or C₁-C₂), substituted (e.g., substituted with a substituent group, a size-limited substituent group, or lower substituent group) or unsubstituted heteroalkyl (e.g., 2 to 20 membered, 2 to 12 membered, 2 to 8 membered, 2 to 6 membered, 4 to 6 membered, 2 to 3 membered, or 4 to 5 membered), substituted (e.g., substituted with a substituent group, a size-limited substituent group, or lower substituent group) or unsubstituted cycloalkyl (e.g., C₃-C₁₀, C₃-C₈, C₃-C₆, C₄-C₆, or C₅-C₆), substituted (e.g., substituted with a substituent group, a size-limited substituent group, or lower substituent group) or unsubstituted heterocycloalkyl (e.g., 3 to 10 membered, 3 to 8 membered, 3 to 6 membered, 4 to 6 membered, 4 to 5 membered, or 5 to 6 membered), substituted (e.g., substituted with a substituent group, a size-limited substituent group, or lower substituent group) or unsubstituted aryl (e.g., C₆-C₁₂, C₆-C₁₀, or phenyl), or substituted (e.g., substituted with a substituent group, a size-limited substituent group, or lower substituent group) or unsubstituted heteroaryl (e.g., 5 to 12 membered, 5 to 10 membered, 5 to 9 membered, or 5 to 6 membered). In embodiments, R^{1E} is independently substituted (e.g., substituted with a substituent group, a size-limited substituent group, or lower substituent group) alkyl (e.g., C₁-C₂₀, C₁-C₁₂, C₁-C₈, C₁-C₆, C₁-C₄, or C₁-C₂), substituted (e.g., substituted with a substituent group, a size-limited substituent group, or lower substituent group) heteroalkyl (e.g., 2 to 20 membered, 2 to 12 membered, 2 to 8 membered, 2 to 6 membered, 4 to 6 membered, 2 to 3 membered, or 4 to 5 membered), substituted (e.g., substituted with a substituent group, a size-limited substituent group, or lower substituent group) cycloalkyl (e.g., C₃-C₁₀, C₃-C₈, C₃-C₆, C₄-C₆, or C₅-C₆), substituted (e.g., substituted with a substituent group, a size-limited substituent group, or lower substituent group) heterocycloalkyl (e.g., 3 to 10 membered, 3 to 8 membered, 3 to 6 membered, 4 to 6 membered, 4 to 5 membered, or 5 to 6 membered), substituted (e.g., substituted with a substituent group, a size-limited substituent group, or lower substituent group) aryl (e.g., C₆-C₁₂, C₆-C₁₀, or phenyl), or substituted (e.g., substituted with a substituent group, a size-limited substituent group, or lower substituent group) heteroaryl (e.g., 5 to 12 membered, 5 to 10 membered, 5 to 9 membered, or 5 to 6 membered). In embodiments, R^{1E} is independently unsubstituted alkyl (e.g., C₁-C₂₀, C₁-C₁₂, C₁-C₈, C₁-C₆, C₁-C₄, or C₁-C₂), unsubstituted heteroalkyl (e.g., 2 to 20 membered, 2 to 12 membered, 2 to 8 membered, 2 to 6 membered, 4 to 6 membered, 2 to 3 membered, or 4 to 5 membered), unsubstituted cycloalkyl (e.g., C₃-C₁₀, C₃-C₈, C₃-C₆, C₄-C₆, or C₅-C₆), unsubstituted heterocycloalkyl (e.g., 3 to 10 membered, 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). In embodiments, when R^{1E} is substituted, R^{1E} is substituted with a substituent group. In embodiments, when R^{1E} is substituted, R^{1E} is substituted with a size-limited substituent group. In embodiments, when R^{1E} is substituted, R^{1E} is substituted with a lower substituent group.

L³ is independently a bond, —N(R²³)—, —O—, —S—, —C(O)—, —N(R²³)C(O)—, —C(O)N(R²⁴)—, —N(R²³)C(O)N(R²⁴)—, —C(O)O—, —OC(O)—, —N(R²³)C(O)O—, —OC(O)N(R²⁴)—, —OPO₂—O—, —O—P(O)(S)—O—, —O—P(O)(R²⁵)—O—, —O—P(S)(R²⁵)—O—, —O—P(O)(NR²³R²⁴)—N—, —O—P(S)(NR²³R²⁴)—N—, —O—P(O)(NR²³R²⁴)—O—, —O—P(S)(NR²³R²⁴)—O—, —P(O)(NR²³R²⁴)—N—, —P(S)(NR²³R²⁴)—N—, —P(O)(NR²³R²⁴)—O—, —P(S)(NR²³R²⁴)—O—, —S—S—, substituted (e.g., substituted with a substituent group, a size-limited substituent group, or lower substituent group) or unsubstituted alkylene (e.g., C₁-C₂₀, C₁-C₁₂, C₁-C₈, C₁-C₆, C₁-C₄, or C₁-C₂), substituted (e.g., substituted with a substituent group, a size-limited substituent group, or lower substituent group) or unsubstituted heteroalkylene (e.g., 2 to 20 membered, 2 to 12 membered, 2 to 8 membered, 2 to 6 membered, 4 to 6 membered, 2 to 3 membered, or 4 to 5 membered), substituted (e.g., substituted with a substituent group, a size-limited substituent group, or lower substituent group) or unsubstituted cycloalkylene (e.g., C₃-C₁₀, C₃-C₈, C₃-C₆, C₄-C₆, or C₅-C₆), substituted (e.g., substituted with a substituent group, a size-limited substituent group, or lower substituent group) or unsubstituted heterocycloalkylene (e.g., 3 to 10 membered, 3 to 8 membered, 3 to 6 membered, 4 to 6 membered, 4 to 5 membered, or 5 to 6 membered), substituted (e.g., substituted with a substituent group, a size-limited substituent group, or lower substituent group) or unsubstituted arylene (e.g., C₆-C₁₂, C₆-C₁₀, or phenyl), or substituted (e.g., substituted with a substituent group, a size-limited substituent group, or lower substituent group) or unsubstituted heteroarylene (e.g., 5 to 12 membered, 5 to 10 membered, 5 to 9 membered, or 5 to 6 membered). In embodiments, L³ is independently a bond, a —N(R²³)—, —O—, —S—, —C(O)—, —N(R²³)C(O)—, —C(O)N(R²⁴)—, —N(R²³)C(O)N(R²⁴)—, —C(O)O—, —OC(O)—, —N(R²³)C(O)O—, —OC(O)N(R²⁴)—, —OPO₂—O—, —O—P(O)(S)—O—, —O—P(O)(R²⁵)—O—, —O—P(S)(R²⁵)—O—, —O—P(O)(NR²³R²⁴)—N—, —O—P(S)(NR²³R²⁴)—N—, —O—P(O)(NR²³ (e.g., substituted with a substituent group, a size-limited substituent group, or lower substituent group) cycloalkylene (e.g., C₃-C₁₀, C₃-C₈, C₃-C₆, C₄-C₆, or C₅-C₆), substituted (e.g., substituted with a substituent group, a size-limited substituent group, or lower substituent group) heterocycloalkylene (e.g., 3 to 10 membered, 3 to 8 membered, 3 to 6 membered, 4 to 6 membered, 4 to 5 membered, or 5 to 6 membered), substituted (e.g., substituted with a substituent group, a size-limited substituent group, or lower substituent group) arylene (e.g., C₆-C₁₂, C₆-C₁₀, or phenyl), or substituted (e.g., substituted with a substituent group, a size-limited substituent group, or lower substituent group) heteroarylene (e.g., 5 to 12 membered, 5 to 10 membered, 5 to 9 membered, or 5 to 6 membered). In embodiments, L³ is independently a bond, —N(R²³)—, —O—, —S—, —C(O)—, —N(R²³)C(O)—, —C(O)N(R²⁴)—, —N(R²³)C(O)N(R²⁴)—, —C(O)O—, —OC(O)—, —N(R²³)C(O)O—, —OC(O)N(R²⁴)—, —OPO₂—O—, —O—P(O)(S)—O—, —O—P(O)(R²⁵)—O—, —O—P(S)(R²⁵)—O—, —O—P(O)(NR²³R²⁴)—N—, —O—P(S)(NR²³R²⁴)—N—, —O—P(O)(NR²³R²⁴)—O—, —O—P(S)(NR²³R²⁴)—O—, —P(O)(NR²³R²⁴)—N—, —P(S)(NR²³R²⁴)—N—, —P(O)(NR²³R²⁴)—O—, —P(S)(NR²³R²⁴)—O—, —S—S—, unsubstituted alkylene (e.g., C₁-C₂₀, C₁-C₁₂, C₁-C₈, C₁-C₆, C₁-C₄, or C₁-C₂), unsubstituted heteroalkylene (e.g., 2 to 20 membered, 2 to 12 membered, 2 to 8 membered, 2 to 6 membered, 4 to 6 membered, 2 to 3 membered, or 4 to 5 membered), unsubstituted cycloalkylene (e.g., C₃-C₁₀, C₃-C₈, C₃-C₆, C₄-C₆, or C₅-C₆), unsubstituted heterocycloalkylene (e.g., 3 to 10 membered, 3 to 8 membered, 3 to 6 membered, 4 to 6 membered, 4 to 5 membered, or 5 to 6 membered), unsubstituted arylene (e.g., C₆-C₁₂, C₆-C₁₀, or phenyl), or unsubstituted heteroarylene (e.g., 5 to 12 membered, 5 to 10 membered, 5 to 9 membered, or 5 to 6 membered). In embodiments, when L³ is substituted, L³ is substituted with a substituent group. In embodiments, when L³ is substituted, L³ is substituted with a size-limited substituent group. In embodiments, when L³ is substituted, L³ is substituted with a lower substituent group.

L⁴ is independently a bond, —N(R²³)—, —O—, —S—, —C(O)—, —N(R²³)C(O)—, —C(O)N(R²⁴)—, —N(R²³)C(O)N(R²⁴)—, —C(O)O—, —OC(O)—, —N(R²³)C(O)O—, —OC(O)N(R²⁴)—, —OPO₂—O—, —O—P(O)(S)—O—, —O—P(O)(R²⁵)—O—, —O—P(S)(R²⁵)—O—, —O—P(O)(NR²³R²⁴)—N—, —O—P(S)(NR²³R²⁴)—N—, —O—P(O)(NR²³R²⁴)—O—, —O—P(S)(NR²³R²⁴)—O—, —P(O)(NR²³R²⁴)—N—, —P(S)(NR²³R²⁴)—N—, —P(O)(NR²³R²⁴)—O—, —P(S)(NR²³R²⁴)—O—, —S—S—, substituted (e.g., substituted with a substituent group, a size-limited substituent group, or lower substituent group) or unsubstituted alkylene (e.g., C₁-C₂₀, C₁-C₁₂, C₁-C₈, C₁-C₆, C₁-C₄, or C₁-C₂), substituted (e.g., substituted with a substituent group, a size-limited substituent group, or lower substituent group) or unsubstituted heteroalkylene (e.g., 2 to 20 membered, 2 to 12 membered, 2 to 8 membered, 2 to 6 membered, 4 to 6 membered, 2 to 3 membered, or 4 to 5 membered), substituted (e.g., substituted with a substituent group, a size-limited substituent group, or lower substituent group) or unsubstituted cycloalkylene (e.g., C₃-C₁₀, C₃-C₈, C₃-C₆, C₄-C₆, or C₅-C₆), substituted (e.g., substituted with a substituent group, a size-limited substituent group, or lower substituent group) or unsubstituted heterocycloalkylene (e.g., 3 to 10 membered, 3 to 8 membered, 3 to 6 membered, 4 to 6 membered, 4 to 5 membered, or 5 to 6 membered), substituted (e.g., substituted with a substituent group, a size-limited substituent group, or lower substituent group) or unsubstituted arylene (e.g., C₆-C₁₂, C₆-C₁₀, or phenyl), or substituted (e.g., substituted with a substituent group, a size-limited substituent group, or lower substituent group) or unsubstituted heteroarylene (e.g., 5 to 12 membered, 5 to 10 membered, 5 to 9 membered, or 5 to 6 membered). In embodiments, L⁴ is a bond, —N(R²³)—, —O—, —S—, —C(O)—, —N(R²³)C(O)—, —C(O)N(R²⁴)—, —N(R²³)C(O)N(R²⁴)—, —C(O)O—, —OC(O)—, —N(R²³)C(O)O—, —OC(O)N(R²⁴)—, —OPO₂—O—, —O—P(O)(S)—O—, —O—P(O)(R²⁵)—O—, —O—P(S)(R²⁵)—O—, —O—P(O)(NR²³R²⁴)—N—, —O—P(S)(NR²³R²⁴)—N—, —O—P(O)(NR²³R²⁴)—O—, —O—P(S)(NR²³R²⁴)—O—, —P(O)(NR²³R²⁴)—N—, —P(S)(NR²³R²⁴)—N—, —P(O)(NR²³R²⁴)—O—, —P(S)(NR²³R²⁴)—O—, —S—S—, substituted (e.g., substituted with a substituent group, a size-limited substituent group, or lower substituent group) alkylene (e.g., C₁-C₂₀, C₁-C₁₂, C₁-C₈, C₁-C₆, C₁-C₄, or C₁-C₂), substituted (e.g., substituted with a substituent group, a size-limited substituent group, or lower substituent group) heteroalkylene (e.g., 2 to 20 membered, 2 to 12 membered, 2 to 8 membered, 2 to 6 membered, 4 to 6 membered, 2 to 3 membered, or 4 to 5 membered), substituted (e.g., substituted with a substituent group, a size-limited substituent group, or lower substituent group) cycloalkylene (e.g., C₃-C₁₀, C₃-C₈, C₃-C₆, C₄-C₆, or C₅-C₆), substituted (e.g., substituted with a substituent group, a size-limited substituent group, or lower substituent group) heterocycloalkylene (e.g., 3 to 10 membered, 3 to 8 membered, 3 to 6 membered, 4 to 6 membered, 4 to 5 membered, or 5 to 6 membered), substituted (e.g., substituted with a substituent group, a size-limited substituent group, or lower substituent group) arylene (e.g., C₆-C₁₂, C₆-C₁₀, or phenyl), or substituted (e.g., substituted with a substituent group, a size-limited substituent group, or lower substituent group) heteroarylene (e.g., 5 to 12 membered, 5 to 10 membered, 5 to 9 membered, or 5 to 6 membered). In embodiments, L⁴ is a bond, —N(R²³)—, —O—, —S—, —C(O)—, —N(R²³)C(O)—, —C(O)N(R²⁴)—, —N(R²³)C(O)N(R²⁴)—, —C(O)O—, —OC(O)—, —N(R²³)C(O)O—, —OC(O)N(R²⁴)—, —OPO₂—O—, —O—P(O)(S)—O—, —O—P(O)(R²⁵)—O—, —O—P(S)(R²⁵)—O—, —O—P(O)(NR²³R²⁴)—N—, —O—P(S)(NR²³R²⁴)—N—, —O—P(O)(NR²³R²⁴)—O—, —O—P(S)(NR²³R²⁴)—O—, —P(O)(NR²³R²⁴)—N—, —P(S)(NR²³R²⁴)—N—, —P(O)(NR²³R²⁴)—O—, —P(S)(NR²³R²⁴)—O—, —S—S—, unsubstituted alkylene (e.g., C₁-C₂₀, C₁-C₁₂, C₁-C₈, C₁-C₆, C₁-C₄, or C₁-C₂), unsubstituted heteroalkylene (e.g., 2 to 20 membered, 2 to 12 membered, 2 to 8 membered, 2 to 6 membered, 4 to 6 membered, 2 to 3 membered, or 4 to 5 membered), unsubstituted cycloalkylene (e.g., C₃-C₁₀, C₃-C₈, C₃-C₆, C₄-C₆, or C₅-C₆), unsubstituted heterocycloalkylene (e.g., 3 to 10 membered, 3 to 8 membered, 3 to 6 membered, 4 to 6 membered, 4 to 5 membered, or 5 to 6 membered), unsubstituted arylene (e.g., C₆-C₁₂, C₆-C₁₀, or phenyl), or unsubstituted heteroarylene (e.g., 5 to 12 membered, 5 to 10 membered, 5 to 9 membered, or 5 to 6 membered). In embodiments, when L⁴ is substituted, L⁴ is substituted with a substituent group. In embodiments, when L⁴ is substituted, L⁴ is substituted with a size-limited substituent group. In embodiments, when L⁴ is substituted, L⁴ is substituted with a lower substituent group.

R²³ is independently hydrogen or unsubstituted alkyl (e.g., C₁-C₂₃, C₁-C₁₂, C₁-C₈, C₁-C₆, C₁-C₄, or C₁-C₂). In embodiments, R²³ is independently hydrogen. In embodiments, R²³ is independently unsubstituted C₁-C₂₃ alkyl. In embodiments, R²³ is independently hydrogen or unsubstituted C₁-C₁₂ alkyl. In embodiments, R²³ is independently hydrogen or unsubstituted C₁-C₁₀ alkyl. In embodiments, R²³ is independently hydrogen or unsubstituted C₁-C₈ alkyl. In embodiments, R²³ is independently hydrogen or unsubstituted C₁-C₆ alkyl. In embodiments, R²³ is independently hydrogen or unsubstituted C₁-C₄ alkyl. In embodiments, R²³ is independently hydrogen or unsubstituted C₁-C₂ alkyl.

R²⁴ is independently hydrogen or unsubstituted alkyl (e.g., C₁-C₂₃, C₁-C₁₂, C₁-C₈, C₁-C₆, C₁-C₄, or C₁-C₂). In embodiments, R²⁴ is independently hydrogen. In embodiments, R²⁴ is independently unsubstituted C₁-C₂₃ alkyl. In embodiments, R²⁴ is independently hydrogen or unsubstituted C₁-C₁₂ alkyl. In embodiments, R²⁴ is independently hydrogen or unsubstituted C₁-C₁₀ alkyl. In embodiments, R²⁴ is independently hydrogen or unsubstituted C₁-C₈ alkyl. In embodiments, R²⁴ is independently hydrogen or unsubstituted C₁-C₆ alkyl. In embodiments, R²⁴ is independently hydrogen or unsubstituted C₁-C₄ alkyl. In embodiments, R²⁴ is independently hydrogen or unsubstituted C₁-C₂ alkyl.

R²⁵ is independently hydrogen or unsubstituted alkyl (e.g., C₁-C₂₃, C₁-C₁₂, C₁-C₈, C₁-C₆, C₁-C₄, or C₁-C₂). In embodiments, R²⁵ is independently hydrogen. In embodiments, R²⁵ is independently unsubstituted C₁-C₂₃ alkyl. In embodiments, R²⁵ is independently hydrogen or unsubstituted C₁-C₁₂ alkyl. In embodiments, R²⁵ is independently hydrogen or unsubstituted C₁-C₁₀ alkyl. In embodiments, R²⁵ is independently hydrogen or unsubstituted C₁-C₈ alkyl. In embodiments, R²⁵ is independently hydrogen or unsubstituted C₁-C₆ alkyl. In embodiments, R²⁵ is independently hydrogen or unsubstituted C₁-C₄ alkyl. In embodiments, R²⁵ is independently hydrogen or unsubstituted C₁-C₂ alkyl.

L⁵ is independently a bond, —NH—, —O—, —S—, —C(O)—, —NHC(O)—, —NHC(O)NH—, —C(O)O—, —OC(O)—, —C(O)NH—, substituted (e.g., substituted with a substituent group, a size-limited substituent group, or lower substituent group) or unsubstituted alkylene (e.g., C₁-C₂₀, C₁-C₁₂, C₁-C₈, C₁-C₆, C₁-C₄, or C₁-C₂), substituted (e.g., substituted with a substituent group, a size-limited substituent group, or lower substituent group) or unsubstituted heteroalkylene (e.g., 2 to 20 membered, 2 to 12 membered, 2 to 8 membered, 2 to 6 membered, 4 to 6 membered, 2 to 3 membered, or 4 to 5 membered), substituted (e.g., substituted with a substituent group, a size-limited substituent group, or lower substituent group) or unsubstituted cycloalkylene (e.g., C₃-C₁₀, C₃-C₈, C₃-C₆, C₄-C₆, or C₅-C₆), substituted (e.g., substituted with a substituent group, a size-limited substituent group, or lower substituent group) or unsubstituted heterocycloalkylene (e.g., 3 to 10 membered, 3 to 8 membered, 3 to 6 membered, 4 to 6 membered, 4 to 5 membered, or 5 to 6 membered), substituted (e.g., substituted with a substituent group, a size-limited substituent group, or lower substituent group) or unsubstituted arylene (e.g., C₆-C₁₂, C₆-C₁₀, or phenyl), or substituted (e.g., substituted with a substituent group, a size-limited substituent group, or lower substituent group) or unsubstituted heteroarylene (e.g., 5 to 12 membered, 5 to 10 membered, 5 to 9 membered, or 5 to 6 membered). In embodiments, L⁵ is independently a bond, —NH—, —O—, —S—, —C(O)—, —NHC(O)—, —NHC(O)NH—, —C(O)O—, —OC(O)—, —C(O)NH—, substituted (e.g., substituted with a substituent group, a size-limited substituent group, or lower substituent group) alkylene (e.g., C₁-C₂₀, C₁-C₁₂, C₁-C₈, C₁-C₆, C₁-C₄, or C₁-C₂), substituted (e.g., substituted with a substituent group, a size-limited substituent group, or lower substituent group) heteroalkylene (e.g., 2 to 20 membered, 2 to 12 membered, 2 to 8 membered, 2 to 6 membered, 4 to 6 membered, 2 to 3 membered, or 4 to 5 membered), substituted (e.g., substituted with a substituent group, a size-limited substituent group, or lower substituent group) cycloalkylene (e.g., C₃-C₁₀, C₃-C₈, C₃-C₆, C₄-C₆, or C₅-C₆), substituted (e.g., substituted with a substituent group, a size-limited substituent group, or lower substituent group) heterocycloalkylene (e.g., 3 to 10 membered, 3 to 8 membered, 3 to 6 membered, 4 to 6 membered, 4 to 5 membered, or 5 to 6 membered), substituted (e.g., substituted with a substituent group, a size-limited substituent group, or lower substituent group) arylene (e.g., C₆-C₁₂, C₆-C₁₀, or phenyl), or substituted (e.g., substituted with a substituent group, a size-limited substituent group, or lower substituent group) heteroarylene (e.g., 5 to 12 membered, 5 to 10 membered, 5 to 9 membered, or 5 to 6 membered). In embodiments, L⁵ is independently a bond, —NH—, —O—, —S—, —C(O)—, —NHC(O)—, —NHC(O)NH—, —C(O)O—, —OC(O)—, —C(O)NH—, unsubstituted alkylene (e.g., C₁-C₂₀, C₁-C₁₂, C₁-C₈, C₁-C₆, C₁-C₄, or C₁-C₂), unsubstituted heteroalkylene (e.g., 2 to 20 membered, 2 to 12 membered, 2 to 8 membered, 2 to 6 membered, 4 to 6 membered, 2 to 3 membered, or 4 to 5 membered), unsubstituted cycloalkylene (e.g., C₃-C₁₀, C₃-C₈, C₃-C₆, C₄-C₆, or C₅-C₆), unsubstituted heterocycloalkylene (e.g., 3 to 10 membered, 3 to 8 membered, 3 to 6 membered, 4 to 6 membered, 4 to 5 membered, or 5 to 6 membered), unsubstituted arylene (e.g., C₆-C₁₂, C₆-C₁₀, or phenyl), or unsubstituted heteroarylene (e.g., 5 to 12 membered, 5 to 10 membered, 5 to 9 membered, or 5 to 6 membered). In embodiments, when L⁵ is substituted, L⁵ is substituted with a substituent group. In embodiments, when L⁵ is substituted, L⁵ is substituted with a size-limited substituent group. In embodiments, when L⁵ is substituted, L⁵ is substituted with a lower substituent group.

L^{5A} is independently a bond, —NH—, —O—, —S—, —C(O)—, —NHC(O)—, —NHC(O)NH—, —C(O)O—, —OC(O)—, —C(O)NH—, substituted (e.g., substituted with a substituent group, a size-limited substituent group, or lower substituent group) or unsubstituted alkylene (e.g., C₁-C₂₀, C₁-C₁₂, C₁-C₈, C₁-C₆, C₁-C₄, or C₁-C₂), substituted (e.g., substituted with a substituent group, a size-limited substituent group, or lower substituent group) or unsubstituted heteroalkylene (e.g., 2 to 20 membered, 2 to 12 membered, 2 to 8 membered, 2 to 6 membered, 4 to 6 membered, 2 to 3 membered, or 4 to 5 membered), substituted (e.g., substituted with a substituent group, a size-limited substituent group, or lower substituent group) or unsubstituted cycloalkylene (e.g., C₃-C₁₀, C₃-C₈, C₃-C₆, C₄-C₆, or C₅-C₆), substituted (e.g., substituted with a substituent group, a size-limited substituent group, or lower substituent group) or unsubstituted heterocycloalkylene (e.g., 3 to 10 membered, 3 to 8 membered, 3 to 6 membered, 4 to 6 membered, 4 to 5 membered, or 5 to 6 membered), substituted (e.g., substituted with a substituent group, a size-limited substituent group, or lower substituent group) or unsubstituted arylene (e.g., C₆-C₁₂, C₆-C₁₀, or phenyl), or substituted (e.g., substituted with a substituent group, a size-limited substituent group, or lower substituent group) or unsubstituted heteroarylene (e.g., 5 to 12 membered, 5 to 10 membered, 5 to 9 membered, or 5 to 6 membered). In embodiments, L^{5A} is independently a bond, —NH—, —O—, —S—, —C(O)—, —NHC(O)—, —NHC(O)NH—, —C(O)O—, —OC(O)—, —C(O)NH—, substituted (e.g., substituted with a substituent group, a size-limited substituent group, or lower substituent group) alkylene (e.g., C₁-C₂₀, C₁-C₁₂, C₁-C₈, C₁-C₆, C₁-C₄, or C₁-C₂), substituted (e.g., substituted with a substituent group, a size-limited substituent group, or lower substituent group) heteroalkylene (e.g., 2 to 20 membered, 2 to 12 membered, 2 to 8 membered, 2 to 6 membered, 4 to 6 membered, 2 to 3 membered, or 4 to 5 membered), substituted (e.g., substituted with a substituent group, a size-limited substituent group, or lower substituent group) cycloalkylene (e.g., C₃-C₁₀, C₃-C₈, C₃-C₆, C₄-C₆, or C₅-C₆), substituted (e.g., substituted with a substituent group, a size-limited substituent group, or lower substituent group) heterocycloalkylene (e.g., 3 to 10 membered, 3 to 8 membered, 3 to 6 membered, 4 to 6 membered, 4 to 5 membered, or 5 to 6 membered), substituted (e.g., substituted with a substituent group, a size-limited substituent group, or lower substituent group) arylene (e.g., C₆-C₁₂, C₆-C₁₀, or phenyl), or substituted (e.g., substituted with a substituent group, a size-limited substituent group, or lower substituent group) heteroarylene (e.g., 5 to 12 membered, 5 to 10 membered, 5 to 9 membered, or 5 to 6 membered). In embodiments, L^{5A} is independently a bond, —NH—, —O—, —S—, —C(O)—, —NHC(O)—, —NHC(O)NH—, —C(O)O—, —OC(O)—, —C(O)NH—, unsubstituted alkylene (e.g., C₁-C₂₀, C₁-C₁₂, C₁-C₈, C₁-C₆, C₁-C₄, or C₁-C₂), unsubstituted heteroalkylene (e.g., 2 to 20 membered, 2 to 12 membered, 2 to 8 membered, 2 to 6 membered, 4 to 6 membered, 2 to 3 membered, or 4 to 5 membered), unsubstituted cycloalkylene (e.g., C₃-C₁₀, C₃-C₈, C₃-C₆, C₄-C₆, or C₅-C₆), unsubstituted heterocycloalkylene (e.g., 3 to 10 membered, 3 to 8 membered, 3 to 6 membered, 4 to 6 membered, 4 to 5 membered, or 5 to 6 membered), unsubstituted arylene (e.g., C₆-C₁₂, C₆-C₁₀, or phenyl), or unsubstituted heteroarylene (e.g., 5 to 12 membered, 5 to 10 membered, 5 to 9 membered, or 5 to 6 membered). In embodiments, when L^{5A} is substituted, L^{5A} is substituted with a substituent group. In embodiments, when L^{5A} is substituted, L^{5A} is substituted with a size-limited substituent group. In embodiments, when L^{5A} is substituted, L^{5A} is substituted with a lower substituent group.

L^{5B} is independently a bond, —NH—, —O—, —S—, —C(O)—, —NHC(O)—, —NHC(O)NH—, —C(O)O—, —OC(O)—, —C(O)NH—, substituted (e.g., substituted with a substituent group, a size-limited substituent group, or lower substituent group) or unsubstituted alkylene (e.g., C₁-C₂₀, C₁-C₁₂, C₁-C₈, C₁-C₆, C₁-C₄, or C₁-C₂), substituted (e.g., substituted with a substituent group, a size-limited substituent group, or lower substituent group) or unsubstituted heteroalkylene (e.g., 2 to 20 membered, 2 to 12 membered, 2 to 8 membered, 2 to 6 membered, 4 to 6 membered, 2 to 3 membered, or 4 to 5 membered), substituted (e.g., substituted with a substituent group, a size-limited substituent group, or lower substituent group) or unsubstituted cycloalkylene (e.g., C₃-C₁₀, C₃-C₈, C₃-C₆, C₄-C₆, or C₅-C₆), substituted (e.g., substituted with a substituent group, a size-limited substituent group, or lower substituent group) or unsubstituted heterocycloalkylene (e.g., 3 to 10 membered, 3 to 8 membered, 3 to 6 membered, 4 to 6 membered, 4 to 5 membered, or 5 to 6 membered), substituted (e.g., substituted with a substituent group, a size-limited substituent group, or lower substituent group) or unsubstituted arylene (e.g., C₆-C₁₂, C₆-C₁₀, or phenyl), or substituted (e.g., substituted with a substituent group, a size-limited substituent group, or lower substituent group) or unsubstituted heteroarylene (e.g., 5 to 12 membered, 5 to 10 membered, 5 to 9 membered, or 5 to 6 membered). In embodiments, L^{5B} is independently a bond, —NH—, —O—, —S—, —C(O)—, —NHC(O)—, —NHC(O)NH—, —C(O)O—, —OC(O)—, —C(O)NH—, substituted (e.g., substituted with a substituent group, a size-limited substituent group, or lower substituent group) alkylene (e.g., C₁-C₂₀, C₁-C₁₂, C₁-C₈, C₁-C₆, C₁-C₄, or C₁-C₂), substituted (e.g., substituted with a substituent group, a size-limited substituent group, or lower substituent group) heteroalkylene (e.g., 2 to 20 membered, 2 to 12 membered, 2 to 8 membered, 2 to 6 membered, 4 to 6 membered, 2 to 3 membered, or 4 to 5 membered), substituted (e.g., substituted with a substituent group, a size-limited substituent group, or lower substituent group) cycloalkylene (e.g., C₃-C₁₀, C₃-C₈, C₃-C₆, C₄-C₆, or C₅-C₆), substituted (e.g., substituted with a substituent group, a size-limited substituent group, or lower substituent group) heterocycloalkylene (e.g., 3 to 10 membered, 3 to 8 membered, 3 to 6 membered, 4 to 6 membered, 4 to 5 membered, or 5 to 6 membered), substituted (e.g., substituted with a substituent group, a size-limited substituent group, or lower substituent group) arylene (e.g., C₆-C₁₂, C₆-C₁₀, or phenyl), or substituted (e.g., substituted with a substituent group, a size-limited substituent group, or lower substituent group) heteroarylene (e.g., 5 to 12 membered, 5 to 10 membered, 5 to 9 membered, or 5 to 6 membered). In embodiments, L^{5B} is independently a bond, —NH—, —O—, —S—, —C(O)—, —NHC(O)—, —NHC(O)NH—, —C(O)O—, —OC(O)—, —C(O)NH—, unsubstituted alkylene (e.g., C₁-C₂₀, C₁-C₁₂, C₁-C₈, C₁-C₆, C₁-C₄, or C₁-C₂), unsubstituted heteroalkylene (e.g., 2 to 20 membered, 2 to 12 membered, 2 to 8 membered, 2 to 6 membered, 4 to 6 membered, 2 to 3 membered, or 4 to 5 membered), unsubstituted cycloalkylene (e.g., C₃-C₁₀, C₃-C₈, C₃-C₆, C₄-C₆, or C₅-C₆), unsubstituted heterocycloalkylene (e.g., 3 to 10 membered, 3 to 8 membered, 3 to 6 membered, 4 to 6 membered, 4 to 5 membered, or 5 to 6 membered), unsubstituted arylene (e.g., C₆-C₁₂, C₆-C₁₀, or phenyl), or unsubstituted heteroarylene (e.g., 5 to 12 membered, 5 to 10 membered, 5 to 9 membered, or 5 to 6 membered). In embodiments, when L^{5B} is substituted, L^{5B} is substituted with a substituent group. In embodiments, when L^{5B} is substituted, L^{5B} is substituted with a size-limited substituent group. In embodiments, when L^{5B} is substituted, L^{5B} is substituted with a lower substituent group.

L^{5C} is independently a bond, —NH—, —O—, —S—, —C(O)—, —NHC(O)—, —NHC(O)NH—, —C(O)O—, —OC(O)—, —C(O)NH—, substituted (e.g., substituted with a substituent group, a size-limited substituent group, or lower substituent group) or unsubstituted alkylene (e.g., C₁-C₂₀, C₁-C₁₂, C₁-C₈, C₁-C₆, C₁-C₄, or C₁-C₂), substituted (e.g., substituted with a substituent group, a size-limited substituent group, or lower substituent group) or unsubstituted heteroalkylene (e.g., 2 to 20 membered, 2 to 12 membered, 2 to 8 membered, 2 to 6 membered, 4 to 6 membered, 2 to 3 membered, or 4 to 5 membered), substituted (e.g., substituted with a substituent group, a size-limited substituent group, or lower substituent group) or unsubstituted cycloalkylene (e.g., C₃-C₁₀, C₃-C₈, C₃-C₆, C₄-C₆, or C₅-C₆), substituted (e.g., substituted with a substituent group, a size-limited substituent group, or lower substituent group) or unsubstituted heterocycloalkylene (e.g., 3 to 10 membered, 3 to 8 membered, 3 to 6 membered, 4 to 6 membered, 4 to 5 membered, or 5 to 6 membered), substituted (e.g., substituted with a substituent group, a size-limited substituent group, or lower substituent group) or unsubstituted arylene (e.g., C₆-C₁₂, C₆-C₁₀, or phenyl), or substituted (e.g., substituted with a substituent group, a size-limited substituent group, or lower substituent group) or unsubstituted heteroarylene (e.g., 5 to 12 membered, 5 to 10 membered, 5 to 9 membered, or 5 to 6 membered). In embodiments, L^{5C} is independently a bond, —NH—, —O—, —S—, —C(O)—, —NHC(O)—, —NHC(O)NH—, —C(O)O—, —OC(O)—, —C(O)NH—, substituted (e.g., substituted with a substituent group, a size-limited substituent group, or lower substituent group) alkylene (e.g., C₁-C₂₀, C₁-C₁₂, C₁-C₈, C₁-C₆, C₁-C₄, or C₁-C₂), substituted (e.g., substituted with a substituent group, a size-limited substituent group, or lower substituent group) heteroalkylene (e.g., 2 to 20 membered, 2 to 12 membered, 2 to 8 membered, 2 to 6 membered, 4 to 6 membered, 2 to 3 membered, or 4 to 5 membered), substituted (e.g., substituted with a substituent group, a size-limited substituent group, or lower substituent group) cycloalkylene (e.g., C₃-C₁₀, C₃-C₈, C₃-C₆, C₄-C₆, or C₅-C₆), substituted (e.g., substituted with a substituent group, a size-limited substituent group, or lower substituent group) heterocycloalkylene (e.g., 3 to 10 membered, 3 to 8 membered, 3 to 6 membered, 4 to 6 membered, 4 to 5 membered, or 5 to 6 membered), substituted (e.g., substituted with a substituent group, a size-limited substituent group, or lower substituent group) arylene (e.g., C₆-C₁₂, C₆-C₁₀, or phenyl), or substituted (e.g., substituted with a substituent group, a size-limited substituent group, or lower substituent group) heteroarylene (e.g., 5 to 12 membered, 5 to 10 membered, 5 to 9 membered, or 5 to 6 membered). In embodiments, L^{5C} is independently a bond, —NH—, —O—, —S—, —C(O)—, —NHC(O)—, —NHC(O)NH—, —C(O)O—, —OC(O)—, —C(O)NH—, unsubstituted alkylene (e.g., C₁-C₂₀, C₁-C₁₂, C₁-C₈, C₁-C₆, C₁-C₄, or C₁-C₂), unsubstituted heteroalkylene (e.g., 2 to 20 membered, 2 to 12 membered, 2 to 8 membered, 2 to 6 membered, 4 to 6 membered, 2 to 3 membered, or 4 to 5 membered), unsubstituted cycloalkylene (e.g., C₃-C₁₀, C₃-C₈, C₃-C₆, C₄-C₆, or C₅-C₆), unsubstituted heterocycloalkylene (e.g., 3 to 10 membered, 3 to 8 membered, 3 to 6 membered, 4 to 6 membered, 4 to 5 membered, or 5 to 6 membered), unsubstituted arylene (e.g., C₆-C₁₂, C₆-C₁₀, or phenyl), or unsubstituted heteroarylene (e.g., 5 to 12 membered, 5 to 10 membered, 5 to 9 membered, or 5 to 6 membered). In embodiments, when L^{5C} is substituted, L^{5C} is substituted with a substituent group. In embodiments, when L^{5C} is substituted, L^{5C} is substituted with a size-limited substituent group. In embodiments, when L^{5C} is substituted, L^{5C} is substituted with a lower substituent group.

L^{5D} is independently a bond, —NH—, —O—, —S—, —C(O)—, —NHC(O)—, —NHC(O)NH—, —C(O)O—, —OC(O)—, —C(O)NH—, substituted (e.g., substituted with a substituent group, a size-limited substituent group, or lower substituent group) or unsubstituted alkylene (e.g., C₁-C₂₀, C₁-C₁₂, C₁-C₈, C₁-C₆, C₁-C₄, or C₁-C₂), substituted (e.g., substituted with a substituent group, a size-limited substituent group, or lower substituent group) or unsubstituted heteroalkylene (e.g., 2 to 20 membered, 2 to 12 membered, 2 to 8 membered, 2 to 6 membered, 4 to 6 membered, 2 to 3 membered, or 4 to 5 membered), substituted (e.g., substituted with a substituent group, a size-limited substituent group, or lower substituent group) or unsubstituted cycloalkylene (e.g., C₃-C₁₀, C₃-C₈, C₃-C₆, C₄-C₆, or C₅-C₆), substituted (e.g., substituted with a substituent group, a size-limited substituent group, or lower substituent group) or unsubstituted heterocycloalkylene (e.g., 3 to 10 membered, 3 to 8 membered, 3 to 6 membered, 4 to 6 membered, 4 to 5 membered, or 5 to 6 membered), substituted (e.g., substituted with a substituent group, a size-limited substituent group, or lower substituent group) or unsubstituted arylene (e.g., C₆-C₁₂, C₆-C₁₀, or phenyl), or substituted (e.g., substituted with a substituent group, a size-limited substituent group, or lower substituent group) or unsubstituted heteroarylene (e.g., 5 to 12 membered, 5 to 10 membered, 5 to 9 membered, or 5 to 6 membered). In embodiments, L^{5D} is independently a bond, —NH—, —O—, —C(O)—, —NHC(O)—, —NHC(O)NH—, —C(O)O—, —OC(O)—, —C(O)NH—, substituted (e.g., substituted with a substituent group, a size-limited substituent group, or lower substituent group) alkylene (e.g., C₁-C₂₀, C₁-C₁₂, C₁-C₈, C₁-C₆, C₁-C₄, or C₁-C₂), substituted (e.g., substituted with a substituent group, a size-limited substituent group, or lower substituent group) heteroalkylene (e.g., 2 to 20 membered, 2 to 12 membered, 2 to 8 membered, 2 to 6 membered, 4 to 6 membered, 2 to 3 membered, or 4 to 5 membered), substituted (e.g., substituted with a substituent group, a size-limited substituent group, or lower substituent group) cycloalkylene (e.g., C₃-C₁₀, C₃-C₈, C₃-C₆, C₄-C₆, or C₅-C₆), substituted (e.g., substituted with a substituent group, a size-limited substituent group, or lower substituent group) heterocycloalkylene (e.g., 3 to 10 membered, 3 to 8 membered, 3 to 6 membered, 4 to 6 membered, 4 to 5 membered, or 5 to 6 membered), substituted (e.g., substituted with a substituent group, a size-limited substituent group, or lower substituent group) arylene (e.g., C₆-C₁₂, C₆-C₁₀, or phenyl), or substituted (e.g., substituted with a substituent group, a size-limited substituent group, or lower substituent group) heteroarylene (e.g., 5 to 12 membered, 5 to 10 membered, 5 to 9 membered, or 5 to 6 membered). In embodiments, L^{5D} is independently a bond, —NH—, —O—, —S—, —C(O)—, —NHC(O)—, —NHC(O)NH—, —C(O)O—, —OC(O)—, —C(O)NH—, unsubstituted alkylene (e.g., C₁-C₂₀, C₁-C₁₂, C₁-C₈, C₁-C₆, C₁-C₄, or C₁-C₂), unsubstituted heteroalkylene (e.g., 2 to 20 membered, 2 to 12 membered, 2 to 8 membered, 2 to 6 membered, 4 to 6 membered, 2 to 3 membered, or 4 to 5 membered), unsubstituted cycloalkylene (e.g., C₃-C₁₀, C₃-C₈, C₃-C₆, C₄-C₆, or C₅-C₆), unsubstituted heterocycloalkylene (e.g., 3 to 10 membered, 3 to 8 membered, 3 to 6 membered, 4 to 6 membered, 4 to 5 membered, or 5 to 6 membered), unsubstituted arylene (e.g., C₆-C₁₂, C₆-C₁₀, or phenyl), or unsubstituted heteroarylene (e.g., 5 to 12 membered, 5 to 10 membered, 5 to 9 membered, or 5 to 6 membered). In embodiments, when L^{5D} is substituted, L^{5D} is substituted with a substituent group. In embodiments, when L^{5D} is substituted, L^{5D} is substituted with a size-limited substituent group. In embodiments, when L^{5D} is substituted, L^{5D} is substituted with a lower substituent group.

L^{5E} is independently a bond, —NH—, —O—, —S—, —C(O)—, —NHC(O)—, —NHC(O)NH—, —C(O)O—, —OC(O)—, —C(O)NH—, substituted (e.g., substituted with a substituent group, a size-limited substituent group, or lower substituent group) or unsubstituted alkylene (e.g., C₁-C₂₀, C₁-C₁₂, C₁-C₈, C₁-C₆, C₁-C₄, or C₁-C₂), substituted (e.g., substituted with a substituent group, a size-limited substituent group, or lower substituent group) or unsubstituted heteroalkylene (e.g., 2 to 20 membered, 2 to 12 membered, 2 to 8 membered, 2 to 6 membered, 4 to 6 membered, 2 to 3 membered, or 4 to 5 membered), substituted (e.g., substituted with a substituent group, a size-limited substituent group, or lower substituent group) or unsubstituted cycloalkylene (e.g., C₃-C₁₀, C₃-C₈, C₃-C₆, C₄-C₆, or C₅-C₆), substituted (e.g., substituted with a substituent group, a size-limited substituent group, or lower substituent group) or unsubstituted heterocycloalkylene (e.g., 3 to 10 membered, 3 to 8 membered, 3 to 6 membered, 4 to 6 membered, 4 to 5 membered, or 5 to 6 membered), substituted (e.g., substituted with a substituent group, a size-limited substituent group, or lower substituent group) or unsubstituted arylene (e.g., C₆-C₁₂, C₆-C₁₀, or phenyl), or substituted (e.g., substituted with a substituent group, a size-limited substituent group, or lower substituent group) or unsubstituted heteroarylene (e.g., 5 to 12 membered, 5 to 10 membered, 5 to 9 membered, or 5 to 6 membered). In embodiments, L^{5E} is independently a bond, —NH—, —O—, —S—, —C(O)—, —NHC(O)—, —NHC(O)NH—, —C(O)O—, —OC(O)—, —C(O)NH—, substituted (e.g., substituted with a substituent group, a size-limited substituent group, or lower substituent group) alkylene (e.g., C₁-C₂₀, C₁-C₁₂, C₁-C₈, C₁-C₆, C₁-C₄, or C₁-C₂), substituted (e.g., substituted with a substituent group, a size-limited substituent group, or lower substituent group) heteroalkylene (e.g., 2 to 20 membered, 2 to 12 membered, 2 to 8 membered, 2 to 6 membered, 4 to 6 membered, 2 to 3 membered, or 4 to 5 membered), substituted (e.g., substituted with a substituent group, a size-limited substituent group, or lower substituent group) cycloalkylene (e.g., C₃-C₁₀, C₃-C₈, C₃-C₆, C₄-C₆, or C₅-C₆), substituted (e.g., substituted with a substituent group, a size-limited substituent group, or lower substituent group) heterocycloalkylene (e.g., 3 to 10 membered, 3 to 8 membered, 3 to 6 membered, 4 to 6 membered, 4 to 5 membered, or 5 to 6 membered), substituted (e.g., substituted with a substituent group, a size-limited substituent group, or lower substituent group) arylene (e.g., C₆-C₁₂, C₆-C₁₀, or phenyl), or substituted (e.g., substituted with a substituent group, a size-limited substituent group, or lower substituent group) heteroarylene (e.g., 5 to 12 membered, 5 to 10 membered, 5 to 9 membered, or 5 to 6 membered). In embodiments, L^{5E} is independently a bond, —NH—, —O—, —S—, —C(O)—, —NHC(O)—, —NHC(O)NH—, —C(O)O—, —OC(O)—, —C(O)NH—, unsubstituted alkylene (e.g., C₁-C₂₀, C₁-C₁₂, C₁-C₈, C₁-C₆, C₁-C₄, or C₁-C₂), unsubstituted heteroalkylene (e.g., 2 to 20 membered, 2 to 12 membered, 2 to 8 membered, 2 to 6 membered, 4 to 6 membered, 2 to 3 membered, or 4 to 5 membered), unsubstituted cycloalkylene (e.g., C₃-C₁₀, C₃-C₈, C₃-C₆, C₄-C₆, or C₅-C₆), unsubstituted heterocycloalkylene (e.g., 3 to 10 membered, 3 to 8 membered, 3 to 6 membered, 4 to 6 membered, 4 to 5 membered, or 5 to 6 membered), unsubstituted arylene (e.g., C₆-C₁₂, C₆-C₁₀, or phenyl), or unsubstituted heteroarylene (e.g., 5 to 12 membered, 5 to 10 membered, 5 to 9 membered, or 5 to 6 membered). In embodiments, when L^{5E} is substituted, L^{5E} is substituted with a substituent group. In embodiments, when L^{5E} is substituted, L^{5E} is substituted with a size-limited substituent group. In embodiments, when L^{5E} is substituted, L^{5E} is substituted with a lower substituent group.

L⁶ is independently a bond, —NH—, —O—, —S—, —C(O)—, —NHC(O)—, —NHC(O)NH—, —C(O)O—, —OC(O)—, —C(O)NH—, substituted (e.g., substituted with a substituent group, a size-limited substituent group, or lower substituent group) or unsubstituted alkylene (e.g., C₁-C₂₀, C₁-C₁₂, C₁-C₈, C₁-C₆, C₁-C₄, or C₁-C₂), substituted (e.g., substituted with a substituent group, a size-limited substituent group, or lower substituent group) or unsubstituted heteroalkylene (e.g., 2 to 20 membered, 2 to 12 membered, 2 to 8 membered, 2 to 6 membered, 4 to 6 membered, 2 to 3 membered, or 4 to 5 membered), substituted (e.g., substituted with a substituent group, a size-limited substituent group, or lower substituent group) or unsubstituted cycloalkylene (e.g., C₃-C₁₀, C₃-C₈, C₃-C₆, C₄-C₆, or C₅-C₆), substituted (e.g., substituted with a substituent group, a size-limited substituent group, or lower substituent group) or unsubstituted heterocycloalkylene (e.g., 3 to 10 membered, 3 to 8 membered, 3 to 6 membered, 4 to 6 membered, 4 to 5 membered, or 5 to 6 membered), substituted (e.g., substituted with a substituent group, a size-limited substituent group, or lower substituent group) or unsubstituted arylene (e.g., C₆-C₁₂, C₆-C₁₀, or phenyl), or substituted (e.g., substituted with a substituent group, a size-limited substituent group, or lower substituent group) or unsubstituted heteroarylene (e.g., 5 to 12 membered, 5 to 10 membered, 5 to 9 membered, or 5 to 6 membered). In embodiments, L⁶ is independently a bond, —NH—, —O—, —S—, —C(O)—, —NHC(O)—, —NHC(O)NH—, —C(O)O—, —OC(O)—, —C(O)NH—, substituted (e.g., substituted with a substituent group, a size-limited substituent group, or lower substituent group) alkylene (e.g., C₁-C₂₀, C₁-C₁₂, C₁-C₈, C₁-C₆, C₁-C₄, or C₁-C₂), substituted (e.g., substituted with a substituent group, a size-limited substituent group, or lower substituent group) heteroalkylene (e.g., 2 to 20 membered, 2 to 12 membered, 2 to 8 membered, 2 to 6 membered, 4 to 6 membered, 2 to 3 membered, or 4 to 5 membered), substituted (e.g., substituted with a substituent group, a size-limited substituent group, or lower substituent group) cycloalkylene (e.g., C₃-C₁₀, C₃-C₈, C₃-C₆, C₄-C₆, or C₅-C₆), substituted (e.g., substituted with a substituent group, a size-limited substituent group, or lower substituent group) heterocycloalkylene (e.g., 3 to 10 membered, 3 to 8 membered, 3 to 6 membered, 4 to 6 membered, 4 to 5 membered, or 5 to 6 membered), substituted (e.g., substituted with a substituent group, a size-limited substituent group, or lower substituent group) arylene (e.g., C₆-C₁₂, C₆-C₁₀, or phenyl), or substituted (e.g., substituted with a substituent group, a size-limited substituent group, or lower substituent group) heteroarylene (e.g., 5 to 12 membered, 5 to 10 membered, 5 to 9 membered, or 5 to 6 membered). In embodiments, L⁶ is independently a bond, —NH—, —O—, —S—, —C(O)—, —NHC(O)—, —NHC(O)NH—, —C(O)O—, —OC(O)—, —C(O)NH—, unsubstituted alkylene (e.g., C₁-C₂₀, C₁-C₁₂, C₁-C₈, C₁-C₆, C₁-C₄, or C₁-C₂), unsubstituted heteroalkylene (e.g., 2 to 20 membered, 2 to 12 membered, 2 to 8 membered, 2 to 6 membered, 4 to 6 membered, 2 to 3 membered, or 4 to 5 membered), unsubstituted cycloalkylene (e.g., C₃-C₁₀, C₃-C₈, C₃-C₆, C₄-C₆, or C₅-C₆), unsubstituted heterocycloalkylene (e.g., 3 to 10 membered, 3 to 8 membered, 3 to 6 membered, 4 to 6 membered, 4 to 5 membered, or 5 to 6 membered), unsubstituted arylene (e.g., C₆-C₁₂, C₆-C₁₀, or phenyl), or unsubstituted heteroarylene (e.g., 5 to 12 membered, 5 to 10 membered, 5 to 9 membered, or 5 to 6 membered). In embodiments, when L⁶ is substituted, L⁶ is substituted with a substituent group. In embodiments, when L⁶ is substituted, L⁶ is substituted with a size-limited substituent group. In embodiments, when L⁶ is substituted, L⁶ is substituted with a lower substituent group.

L⁶¹ is independently a bond, —NH—, —O—, —S—, —C(O)—, —NHC(O)—, —NHC(O)NH—, —C(O)O—, —OC(O)—, —C(O)NH—, substituted (e.g., substituted with a substituent group, a size-limited substituent group, or lower substituent group) or unsubstituted alkylene (e.g., C₁-C₂₀, C₁-C₁₂, C₁-C₈, C₁-C₆, C₁-C₄, or C₁-C₂), substituted (e.g., substituted with a substituent group, a size-limited substituent group, or lower substituent group) or unsubstituted heteroalkylene (e.g., 2 to 20 membered, 2 to 12 membered, 2 to 8 membered, 2 to 6 membered, 4 to 6 membered, 2 to 3 membered, or 4 to 5 membered), substituted (e.g., substituted with a substituent group, a size-limited substituent group, or lower substituent group) or unsubstituted cycloalkylene (e.g., C₃-C₁₀, C₃-C₈, C₃-C₆, C₄-C₆, or C₅-C₆), substituted (e.g., substituted with a substituent group, a size-limited substituent group, or lower substituent group) or unsubstituted heterocycloalkylene (e.g., 3 to 10 membered, 3 to 8 membered, 3 to 6 membered, 4 to 6 membered, 4 to 5 membered, or 5 to 6 membered), substituted (e.g., substituted with a substituent group, a size-limited substituent group, or lower substituent group) or unsubstituted arylene (e.g., C₆-C₁₂, C₆-C₁₀, or phenyl), or substituted (e.g., substituted with a substituent group, a size-limited substituent group, or lower substituent group) or unsubstituted heteroarylene (e.g., 5 to 12 membered, 5 to 10 membered, 5 to 9 membered, or 5 to 6 membered). In embodiments, L⁶¹ is independently a bond, —NH—, —O—, —S—, —C(O)—, —NHC(O)—, —NHC(O)NH—, —C(O)O—, —OC(O)—, —C(O)NH—, substituted (e.g., substituted with a substituent group, a size-limited substituent group, or lower substituent group) alkylene (e.g., C₁-C₂₀, C₁-C₁₂, C₁-C₈, C₁-C₆, C₁-C₄, or C₁-C₂), substituted (e.g., substituted with a substituent group, a size-limited substituent group, or lower substituent group) heteroalkylene (e.g., 2 to 20 membered, 2 to 12 membered, 2 to 8 membered, 2 to 6 membered, 4 to 6 membered, 2 to 3 membered, or 4 to 5 membered), substituted (e.g., substituted with a substituent group, a size-limited substituent group, or lower substituent group) cycloalkylene (e.g., C₃-C₁₀, C₃-C₈, C₃-C₆, C₄-C₆, or C₅-C₆), substituted (e.g., substituted with a substituent group, a size-limited substituent group, or lower substituent group) heterocycloalkylene (e.g., 3 to 10 membered, 3 to 8 membered, 3 to 6 membered, 4 to 6 membered, 4 to 5 membered, or 5 to 6 membered), substituted (e.g., substituted with a substituent group, a size-limited substituent group, or lower substituent group) arylene (e.g., C₆-C₁₂, C₆-C₁₀, or phenyl), or substituted (e.g., substituted with a substituent group, a size-limited substituent group, or lower substituent group) heteroarylene (e.g., 5 to 12 membered, 5 to 10 membered, 5 to 9 membered, or 5 to 6 membered). In embodiments, L^{6A} is independently a bond, —NH—, —O—, —S—, —C(O)—, —NHC(O)—, —NHC(O)NH—, —C(O)O—, —OC(O)—, —C(O)NH—, unsubstituted alkylene (e.g., C₁-C₂₀, C₁-C₁₂, C₁-C₈, C₁-C₆, C₁-C₄, or C₁-C₂), unsubstituted heteroalkylene (e.g., 2 to 20 membered, 2 to 12 membered, 2 to 8 membered, 2 to 6 membered, 4 to 6 membered, 2 to 3 membered, or 4 to 5 membered), unsubstituted cycloalkylene (e.g., C₃-C₁₀, C₃-C₈, C₃-C₆, C₄-C₆, or C₅-C₆), unsubstituted heterocycloalkylene (e.g., 3 to 10 membered, 3 to 8 membered, 3 to 6 membered, 4 to 6 membered, 4 to 5 membered, or 5 to 6 membered), unsubstituted arylene (e.g., C₆-C₁₂, C₆-C₁₀, or phenyl), or unsubstituted heteroarylene (e.g., 5 to 12 membered, 5 to 10 membered, 5 to 9 membered, or 5 to 6 membered). In embodiments, when L^{6A} is substituted, L^{6A} is substituted with a substituent group. In embodiments, when L^{6A} is substituted, L^{6A} is substituted with a size-limited substituent group. In embodiments, when L^{6A} is substituted, L^{6A} is substituted with a lower substituent group.

L^{6B} is independently a bond, —NH—, —O—, —S—, —C(O)—, —NHC(O)—, —NHC(O)NH—, —C(O)O—, —OC(O)—, —C(O)NH—, substituted (e.g., substituted with a substituent group, a size-limited substituent group, or lower substituent group) or unsubstituted alkylene (e.g., C₁-C₂₀, C₁-C₁₂, C₁-C₈, C₁-C₆, C₁-C₄, or C₁-C₂), substituted (e.g., substituted with a substituent group, a size-limited substituent group, or lower substituent group) or unsubstituted heteroalkylene (e.g., 2 to 20 membered, 2 to 12 membered, 2 to 8 membered, 2 to 6 membered, 4 to 6 membered, 2 to 3 membered, or 4 to 5 membered), substituted (e.g., substituted with a substituent group, a size-limited substituent group, or lower substituent group) or unsubstituted cycloalkylene (e.g., C₃-C₁₀, C₃-C₈, C₃-C₆, C₄-C₆, or C₅-C₆), substituted (e.g., substituted with a substituent group, a size-limited substituent group, or lower substituent group) or unsubstituted heterocycloalkylene (e.g., 3 to 10 membered, 3 to 8 membered, 3 to 6 membered, 4 to 6 membered, 4 to 5 membered, or 5 to 6 membered), substituted (e.g., substituted with a substituent group, a size-limited substituent group, or lower substituent group) or unsubstituted arylene (e.g., C₆-C₁₂, C₆-C₁₀, or phenyl), or substituted (e.g., substituted with a substituent group, a size-limited substituent group, or lower substituent group) or unsubstituted heteroarylene (e.g., 5 to 12 membered, 5 to 10 membered, 5 to 9 membered, or 5 to 6 membered). In embodiments, L^{6B} is independently a bond, —NH—, —O—, —S—, —C(O)—, —NHC(O)—, —NHC(O)NH—, —C(O)O—, —OC(O)—, —C(O)NH—, substituted (e.g., substituted with a substituent group, a size-limited substituent group, or lower substituent group) alkylene (e.g., C₁-C₂₀, C₁-C₁₂, C₁-C₈, C₁-C₆, C₁-C₄, or C₁-C₂), substituted (e.g., substituted with a substituent group, a size-limited substituent group, or lower substituent group) heteroalkylene (e.g., 2 to 20 membered, 2 to 12 membered, 2 to 8 membered, 2 to 6 membered, 4 to 6 membered, 2 to 3 membered, or 4 to 5 membered), substituted (e.g., substituted with a substituent group, a size-limited substituent group, or lower substituent group) cycloalkylene (e.g., C₃-C₁₀, C₃-C₈, C₃-C₆, C₄-C₆, or C₅-C₆), substituted (e.g., substituted with a substituent group, a size-limited substituent group, or lower substituent group) heterocycloalkylene (e.g., 3 to 10 membered, 3 to 8 membered, 3 to 6 membered, 4 to 6 membered, 4 to 5 membered, or 5 to 6 membered), substituted (e.g., substituted with a substituent group, a size-limited substituent group, or lower substituent group) arylene (e.g., C₆-C₁₂, C₆-C₁₀, or phenyl), or substituted (e.g., substituted with a substituent group, a size-limited substituent group, or lower substituent group) heteroarylene (e.g., 5 to 12 membered, 5 to 10 membered, 5 to 9 membered, or 5 to 6 membered). In embodiments, L^{6B} is independently a bond, —NH—, —O—, —S—, —C(O)—, —NHC(O)—, —NHC(O)NH—, —C(O)O—, —OC(O)—, —C(O)NH—, unsubstituted alkylene (e.g., C₁-C₂₀, C₁-C₁₂, C₁-C₈, C₁-C₆, C₁-C₄, or C₁-C₂), unsubstituted heteroalkylene (e.g., 2 to 20 membered, 2 to 12 membered, 2 to 8 membered, 2 to 6 membered, 4 to 6 membered, 2 to 3 membered, or 4 to 5 membered), unsubstituted cycloalkylene (e.g., C₃-C₁₀, C₃-C₈, C₃-C₆, C₄-C₆, or C₅-C₆), unsubstituted heterocycloalkylene (e.g., 3 to 10 membered, 3 to 8 membered, 3 to 6 membered, 4 to 6 membered, 4 to 5 membered, or 5 to 6 membered), unsubstituted arylene (e.g., C₆-C₁₂, C₆-C₁₀, or phenyl), or unsubstituted heteroarylene (e.g., 5 to 12 membered, 5 to 10 membered, 5 to 9 membered, or 5 to 6 membered). In embodiments, when L^{6B} is substituted, L^{6B} is substituted with a substituent group. In embodiments, when L^{6B} is substituted, L^{6B} is substituted with a size-limited substituent group. In embodiments, when L^{6B} is substituted, L^{6B} is substituted with a lower substituent group.

L^{6C} is independently a bond, —NH—, —O—, —S—, —C(O)—, —NHC(O)—, —NHC(O)NH—, —C(O)O—, —OC(O)—, —C(O)NH—, substituted (e.g., substituted with a substituent group, a size-limited substituent group, or lower substituent group) or unsubstituted alkylene (e.g., C₁-C₂₀, C₁-C₁₂, C₁-C₈, C₁-C₆, C₁-C₄, or C₁-C₂), substituted (e.g., substituted with a substituent group, a size-limited substituent group, or lower substituent group) or unsubstituted heteroalkylene (e.g., 2 to 20 membered, 2 to 12 membered, 2 to 8 membered, 2 to 6 membered, 4 to 6 membered, 2 to 3 membered, or 4 to 5 membered), substituted (e.g., substituted with a substituent group, a size-limited substituent group, or lower substituent group) or unsubstituted cycloalkylene (e.g., C₃-C₁₀, C₃-C₈, C₃-C₆, C₄-C₆, or C₅-C₆), substituted (e.g., substituted with a substituent group, a size-limited substituent group, or lower substituent group) or unsubstituted heterocycloalkylene (e.g., 3 to 10 membered, 3 to 8 membered, 3 to 6 membered, 4 to 6 membered, 4 to 5 membered, or 5 to 6 membered), substituted (e.g., substituted with a substituent group, a size-limited substituent group, or lower substituent group) or unsubstituted arylene (e.g., C₆-C₁₂, C₆-C₁₀, or phenyl), or substituted (e.g., substituted with a substituent group, a size-limited substituent group, or lower substituent group) or unsubstituted heteroarylene (e.g., 5 to 12 membered, 5 to 10 membered, 5 to 9 membered, or 5 to 6 membered). In embodiments, L^{6C} is independently a bond, —NH—, —O—, —S—, —C(O)—, —NHC(O)—, —NHC(O)NH—, —C(O)O—, —OC(O)—, —C(O)NH—, substituted (e.g., substituted with a substituent group, a size-limited substituent group, or lower substituent group) alkylene (e.g., C₁-C₂₀, C₁-C₁₂, C₁-C₈, C₁-C₆, C₁-C₄, or C₁-C₂), substituted (e.g., substituted with a substituent group, a size-limited substituent group, or lower substituent group) heteroalkylene (e.g., 2 to 20 membered, 2 to 12 membered, 2 to 8 membered, 2 to 6 membered, 4 to 6 membered, 2 to 3 membered, or 4 to 5 membered), substituted (e.g., substituted with a substituent group, a size-limited substituent group, or lower substituent group) cycloalkylene (e.g., C₃-C₁₀, C₃-C₈, C₃-C₆, C₄-C₆, or C₅-C₆), substituted (e.g., substituted with a substituent group, a size-limited substituent group, or lower substituent group) heterocycloalkylene (e.g., 3 to 10 membered, 3 to 8 membered, 3 to 6 membered, 4 to 6 membered, 4 to 5 membered, or 5 to 6 membered), substituted (e.g., substituted with a substituent group, a size-limited substituent group, or lower substituent group) arylene (e.g., C₆-C₁₂, C₆-C₁₀, or phenyl), or substituted (e.g., substituted with a substituent group, a size-limited substituent group, or lower substituent group) heteroarylene (e.g., 5 to 12 membered, 5 to 10 membered, 5 to 9 membered, or 5 to 6 membered). In embodiments, L^{6C} is independently a bond, —NH—, —O—, —S—, —C(O)—, —NHC(O)—, —NHC(O)NH—, —C(O)O—, —OC(O)—, —C(O)NH—, unsubstituted alkylene (e.g., C₁-C₂₀, C₁-C₁₂, C₁-C₈, C₁-C₆, C₁-C₄, or C₁-C₂), unsubstituted heteroalkylene (e.g., 2 to 20 membered, 2 to 12 membered, 2 to 8 membered, 2 to 6 membered, 4 to 6 membered, 2 to 3 membered, or 4 to 5 membered), unsubstituted cycloalkylene (e.g., C₃-C₁₀, C₃-C₈, C₃-C₆, C₄-C₆, or C₅-C₆), unsubstituted heterocycloalkylene (e.g., 3 to 10 membered, 3 to 8 membered, 3 to 6 membered, 4 to 6 membered, 4 to 5 membered, or 5 to 6 membered), unsubstituted arylene (e.g., C₆-C₁₂, C₆-C₁₀, or phenyl), or unsubstituted heteroarylene (e.g., 5 to 12 membered, 5 to 10 membered, 5 to 9 membered, or 5 to 6 membered). In embodiments, when L^{6C} is substituted, L^{6C} is substituted with a substituent group. In embodiments, when L^{6C} is substituted, L^{6C} is substituted with a size-limited substituent group. In embodiments, when L^{6C} is substituted, L^{6C} is substituted with a lower substituent group.

L^{6D} is independently a bond, —NH—, —O—, —S—, —C(O)—, —NHC(O)—, —NHC(O)NH—, —C(O)O—, —OC(O)—, —C(O)NH—, substituted (e.g., substituted with a substituent group, a size-limited substituent group, or lower substituent group) or unsubstituted alkylene (e.g., C₁-C₂₀, C₁-C₁₂, C₁-C₈, C₁-C₆, C₁-C₄, or C₁-C₂), substituted (e.g., substituted with a substituent group, a size-limited substituent group, or lower substituent group) or unsubstituted heteroalkylene (e.g., 2 to 20 membered, 2 to 12 membered, 2 to 8 membered, 2 to 6 membered, 4 to 6 membered, 2 to 3 membered, or 4 to 5 membered), substituted (e.g., substituted with a substituent group, a size-limited substituent group, or lower substituent group) or unsubstituted cycloalkylene (e.g., C₃-C₁₀, C₃-C₈, C₃-C₆, C₄-C₆, or C₅-C₆), substituted (e.g., substituted with a substituent group, a size-limited substituent group, or lower substituent group) or unsubstituted heterocycloalkylene (e.g., 3 to 10 membered, 3 to 8 membered, 3 to 6 membered, 4 to 6 membered, 4 to 5 membered, or 5 to 6 membered), substituted (e.g., substituted with a substituent group, a size-limited substituent group, or lower substituent group) or unsubstituted arylene (e.g., C₆-C₁₂, C₆-C₁₀, or phenyl), or substituted (e.g., substituted with a substituent group, a size-limited substituent group, or lower substituent group) or unsubstituted heteroarylene (e.g., 5 to 12 membered, 5 to 10 membered, 5 to 9 membered, or 5 to 6 membered). In embodiments, L^{6D} is independently a bond, —NH—, —O—, —S—, —C(O)—, —NHC(O)—, —NHC(O)NH—, —C(O)O—, —OC(O)—, —C(O)NH—, substituted (e.g., substituted with a substituent group, a size-limited substituent group, or lower substituent group) alkylene (e.g., C₁-C₂₀, C₁-C₁₂, C₁-C₈, C₁-C₆, C₁-C₄, or C₁-C₂), substituted (e.g., substituted with a substituent group, a size-limited substituent group, or lower substituent group) heteroalkylene (e.g., 2 to 20 membered, 2 to 12 membered, 2 to 8 membered, 2 to 6 membered, 4 to 6 membered, 2 to 3 membered, or 4 to 5 membered), substituted (e.g., substituted with a substituent group, a size-limited substituent group, or lower substituent group) cycloalkylene (e.g., C₃-C₁₀, C₃-C₈, C₃-C₆, C₄-C₆, or C₅-C₆), substituted (e.g., substituted with a substituent group, a size-limited substituent group, or lower substituent group) heterocycloalkylene (e.g., 3 to 10 membered, 3 to 8 membered, 3 to 6 membered, 4 to 6 membered, 4 to 5 membered, or 5 to 6 membered), substituted (e.g., substituted with a substituent group, a size-limited substituent group, or lower substituent group) arylene (e.g., C₆-C₁₂, C₆-C₁₀, or phenyl), or substituted (e.g., substituted with a substituent group, a size-limited substituent group, or lower substituent group) heteroarylene (e.g., 5 to 12 membered, 5 to 10 membered, 5 to 9 membered, or 5 to 6 membered). In embodiments, L^{6D} is independently a bond, —NH—, —O—, —S—, —C(O)—, —NHC(O)—, —NHC(O)NH—, —C(O)O—, —OC(O)—, —C(O)NH—, unsubstituted alkylene (e.g., C₁-C₂₀, C₁-C₁₂, C₁-C₈, C₁-C₆, C₁-C₄, or C₁-C₂), unsubstituted heteroalkylene (e.g., 2 to 20 membered, 2 to 12 membered, 2 to 8 membered, 2 to 6 membered, 4 to 6 membered, 2 to 3 membered, or 4 to 5 membered), unsubstituted cycloalkylene (e.g., C₃-C₁₀, C₃-C₈, C₃-C₆, C₄-C₆, or C₅-C₆), unsubstituted heterocycloalkylene (e.g., 3 to 10 membered, 3 to 8 membered, 3 to 6 membered, 4 to 6 membered, 4 to 5 membered, or 5 to 6 membered), unsubstituted arylene (e.g., C₆-C₁₂, C₆-C₁₀, or phenyl), or unsubstituted heteroarylene (e.g., 5 to 12 membered, 5 to 10 membered, 5 to 9 membered, or 5 to 6 membered). In embodiments, when L^{6D} is substituted, L^{6D} is substituted with a substituent group. In embodiments, when L^{6D} is substituted, L^{6D} is substituted with a size-limited substituent group. In embodiments, when L^{6D} is substituted, L^{6D} is substituted with a lower substituent group.

L^{6E} is independently a bond, —NH—, —O—, —S—, —C(O)—, —NHC(O)—, —NHC(O)NH—, —C(O)O—, —OC(O)—, —C(O)NH—, substituted (e.g., substituted with a substituent group, a size-limited substituent group, or lower substituent group) or unsubstituted alkylene (e.g., C₁-C₂₀, C₁-C₁₂, C₁-C₈, C₁-C₆, C₁-C₄, or C₁-C₂), substituted (e.g., substituted with a substituent group, a size-limited substituent group, or lower substituent group) or unsubstituted heteroalkylene (e.g., 2 to 20 membered, 2 to 12 membered, 2 to 8 membered, 2 to 6 membered, 4 to 6 membered, 2 to 3 membered, or 4 to 5 membered), substituted (e.g., substituted with a substituent group, a size-limited substituent group, or lower substituent group) or unsubstituted cycloalkylene (e.g., C₃-C₁₀, C₃-C₈, C₃-C₆, C₄-C₆, or C₅-C₆), substituted (e.g., substituted with a substituent group, a size-limited substituent group, or lower substituent group) or unsubstituted heterocycloalkylene (e.g., 3 to 10 membered, 3 to 8 membered, 3 to 6 membered, 4 to 6 membered, 4 to 5 membered, or 5 to 6 membered), substituted (e.g., substituted with a substituent group, a size-limited substituent group, or lower substituent group) or unsubstituted arylene (e.g., C₆-C₁₂, C₆-C₁₀, or phenyl), or substituted (e.g., substituted with a substituent group, a size-limited substituent group, or lower substituent group) or unsubstituted heteroarylene (e.g., 5 to 12 membered, 5 to 10 membered, 5 to 9 membered, or 5 to 6 membered). In embodiments, L^{6E} is independently a bond, —NH—, —O—, —S—, —C(O)—, —NHC(O)—, —NHC(O)NH—, —C(O)O—, —OC(O)—, —C(O)NH—, substituted (e.g., substituted with a substituent group, a size-limited substituent group, or lower substituent group) alkylene (e.g., C₁-C₂₀, C₁-C₁₂, C₁-C₈, C₁-C₆, C₁-C₄, or C₁-C₂), substituted (e.g., substituted with a substituent group, a size-limited substituent group, or lower substituent group) heteroalkylene (e.g., 2 to 20 membered, 2 to 12 membered, 2 to 8 membered, 2 to 6 membered, 4 to 6 membered, 2 to 3 membered, or 4 to 5 membered), substituted (e.g., substituted with a substituent group, a size-limited substituent group, or lower substituent group) cycloalkylene (e.g., C₃-C₁₀, C₃-C₈, C₃-C₆, C₄-C₆, or C₅-C₆), substituted (e.g., substituted with a substituent group, a size-limited substituent group, or lower substituent group) heterocycloalkylene (e.g., 3 to 10 membered, 3 to 8 membered, 3 to 6 membered, 4 to 6 membered, 4 to 5 membered, or 5 to 6 membered), substituted (e.g., substituted with a substituent group, a size-limited substituent group, or lower substituent group) arylene (e.g., C₆-C₁₂, C₆-C₁₀, or phenyl), or substituted (e.g., substituted with a substituent group, a size-limited substituent group, or lower substituent group) heteroarylene (e.g., 5 to 12 membered, 5 to 10 membered, 5 to 9 membered, or 5 to 6 membered). In embodiments, L^{6E} is independently a bond, —NH—, —O—, —S—, —C(O)—, —NHC(O)—, —NHC(O)NH—, —C(O)O—, —OC(O)—, —C(O)NH—, unsubstituted alkylene (e.g., C₁-C₂₀, C₁-C₁₂, C₁-C₈, C₁-C₆, C₁-C₄, or C₁-C₂), unsubstituted heteroalkylene (e.g., 2 to 20 membered, 2 to 12 membered, 2 to 8 membered, 2 to 6 membered, 4 to 6 membered, 2 to 3 membered, or 4 to 5 membered), unsubstituted cycloalkylene (e.g., C₃-C₁₀, C₃-C₈, C₃-C₆, C₄-C₆, or C₅-C₆), unsubstituted heterocycloalkylene (e.g., 3 to 10 membered, 3 to 8 membered, 3 to 6 membered, 4 to 6 membered, 4 to 5 membered, or 5 to 6 membered), unsubstituted arylene (e.g., C₆-C₁₂, C₆-C₁₀, or phenyl), or unsubstituted heteroarylene (e.g., 5 to 12 membered, 5 to 10 membered, 5 to 9 membered, or 5 to 6 membered). In embodiments, when L^{6E} is substituted, L^{6E} is substituted with a substituent group. In embodiments, when L^{6E} is substituted, L^{6E} is substituted with a size-limited substituent group. In embodiments, when L^{6E} is substituted, L^{6E} is substituted with a lower substituent group.

In embodiments, L⁷ is independently substituted (e.g., substituted with a substituent group, a size-limited substituent group, or lower substituent group) or unsubstituted alkylene (e.g., C₁-C₂₀, C₁-C₁₂, C₁-C₈, C₁-C₆, C₁-C₄, or C₁-C₂). In embodiments, L⁷ is independently substituted (e.g., substituted with a substituent group, a size-limited substituent group, or lower substituent group) alkylene (e.g., C₁-C₂₀, C₁-C₁₂, C₁-C₈, C₁-C₆, C₁-C₄, or C₁-C₂). In embodiments, L⁷ is independently unsubstituted alkylene (e.g., C₁-C₂₀, C₁-C₁₂, C₁-C₈, C₁-C₆, C₁-C₄, or C₁-C₂).

In embodiments, L⁷ is independently substituted (e.g., substituted with a substituent group, a size-limited substituent group, or lower substituent group) or unsubstituted heteroalkylene (e.g., 2 to 20 membered, 2 to 12 membered, 2 to 10 membered, 2 to 8 membered, 2 to 6 membered, or 2 to 4 membered). In embodiments, L⁷ is independently substituted (e.g., substituted with a substituent group, a size-limited substituent group, or lower substituent group) heteroalkylene (e.g., 2 to 20 membered, 2 to 12 membered, 2 to 10 membered, 2 to 8 membered, 2 to 6 membered, or 2 to 4 membered). In embodiments, L⁷ is independently unsubstituted heteroalkylene (e.g., 2 to 20 membered, 2 to 12 membered, 2 to 10 membered, 2 to 8 membered, 2 to 6 membered, or 2 to 4 membered). In embodiments, L⁷ is independently substituted (e.g., substituted with a substituent group, a size-limited substituent group, or lower substituent group) or unsubstituted heteroalkenylene (e.g., 2 to 20 membered, 2 to 12 membered, 2 to 10 membered, 2 to 8 membered, 2 to 6 membered, or 2 to 4 membered). In embodiments, L⁷ is independently substituted (e.g., substituted with a substituent group, a size-limited substituent group, or lower substituent group) heteroalkenylene (e.g., 2 to 20 membered, 2 to 12 membered, 2 to 10 membered, 2 to 8 membered, 2 to 6 membered, or 2 to 4 membered). In embodiments, L⁷ is independently unsubstituted heteroalkenylene (e.g., 2 to 20 membered, 2 to 12 membered, 2 to 10 membered, 2 to 8 membered, 2 to 6 membered, or 2 to 4 membered). In embodiments, when L⁷ is substituted, L⁷ is substituted with a substituent group. In embodiments, when L⁷ is substituted, L⁷ is substituted with a size-limited substituent group. In embodiments, when L⁷ is substituted, L⁷ is substituted with a lower substituent group.

In embodiments, R¹ is unsubstituted alkyl (e.g., C₁-C₂₅, C₁-C₂₀, C₁-C₁₂, C₁-C₈, C₁-C₆, C₁-C₄, or C₁-C₂). In embodiments, R¹ is unsubstituted C₁-C₂₅ alkyl. In embodiments, R¹ is unsubstituted C₁-C₂₀ alkyl. In embodiments, R¹ is unsubstituted C₁-C₁₂ alkyl. In embodiments, R¹ is unsubstituted C₁-C₈ alkyl. In embodiments, R¹ is unsubstituted C₁-C₆ alkyl. In embodiments, R¹ is unsubstituted C₁-C₄ alkyl. In embodiments, R¹ is unsubstituted C₁-C₂ alkyl.

In embodiments, R¹ is unsubstituted branched alkyl (e.g., C₁-C₂₅, C₁-C₂₀, C₁-C₁₂, C₁-C₈, C₁-C₆, C₁-C₄, or C₁-C₂). In embodiments, R¹ is unsubstituted branched C₁-C₂₅ alkyl.

In embodiments, R¹ is unsubstituted branched C₁-C₂₀ alkyl. In embodiments, R¹ is unsubstituted branched C₁-C₁₂ alkyl. In embodiments, R¹ is unsubstituted branched C₁-C₈ alkyl. In embodiments, R¹ is unsubstituted branched C₁-C₆ alkyl. In embodiments, R¹ is unsubstituted branched C₁-C₄ alkyl. In embodiments, R¹ is unsubstituted branched C₁-C₂ alkyl.

In embodiments, R¹ is unsubstituted unbranched alkyl (e.g., C₁-C₂₅, C₁-C₂₀, C₁-C₁₂, C₁-C₈, C₁-C₆, C₁-C₄, or C₁-C₂). In embodiments, R¹ is unsubstituted unbranched C₁-C₂₅ alkyl.

In embodiments, R¹ is unsubstituted unbranched C₁-C₂₀ alkyl. In embodiments, R¹ is unsubstituted unbranched C₁-C₁₂ alkyl. In embodiments, R¹ is unsubstituted unbranched C₁-C₈ alkyl. In embodiments, R¹ is unsubstituted unbranched C₁-C₆ alkyl. In embodiments, R¹ is unsubstituted unbranched C₁-C₄ alkyl. In embodiments, R¹ is unsubstituted unbranched C₁-C₂ alkyl.

In embodiments, R¹ is unsubstituted branched saturated alkyl (e.g., C₁-C₂₅, C₁-C₂₀, C₁-C₁₂, C₁-C₈, C₁-C₆, C₁-C₄, or C₁-C₂). In embodiments, R¹ is unsubstituted branched saturated C₁-C₂₅ alkyl. In embodiments, R¹ is unsubstituted branched saturated C₁-C₂₀ alkyl.

In embodiments, R¹ is unsubstituted branched saturated C₁-C₁₂ alkyl. In embodiments, R¹ is unsubstituted branched saturated C₁-C₈ alkyl. In embodiments, R¹ is unsubstituted branched saturated C₁-C₆ alkyl. In embodiments, R¹ is unsubstituted branched saturated C₁-C₄ alkyl. In embodiments, R¹ is unsubstituted branched saturated C₁-C₂ alkyl.

In embodiments, R¹ is unsubstituted branched unsaturated alkyl (e.g., C₁-C₂₅, C₁-C₂₀, C₁-C₁₂, C₁-C₈, C₁-C₆, C₁-C₄, or C₁-C₂). In embodiments, R¹ is unsubstituted branched unsaturated C₁-C₂₅ alkyl. In embodiments, R¹ is unsubstituted branched unsaturated C₁-C₂₀ alkyl. In embodiments, R¹ is unsubstituted branched unsaturated C₁-C₁₂ alkyl. In embodiments, R¹ is unsubstituted branched unsaturated C₁-C₈ alkyl. In embodiments, R¹ is unsubstituted branched unsaturated C₁-C₆ alkyl. In embodiments, R¹ is unsubstituted branched unsaturated C₁-C₄ alkyl. In embodiments, R¹ is unsubstituted branched saturated C₁-C₂ alkyl.

In embodiments, R¹ is unsubstituted unbranched saturated alkyl (e.g., C₁-C₂₅, C₁-C₂₀, C₁-C₁₂, C₁-C₈, C₁-C₆, C₁-C₄, or C₁-C₂). In embodiments, R¹ is unsubstituted unbranched saturated C₁-C₂₅ alkyl. In embodiments, R¹ is unsubstituted unbranched saturated C₁-C₂₀ alkyl. In embodiments, R¹ is unsubstituted unbranched saturated C₁-C₁₂ alkyl. In embodiments, R¹ is unsubstituted unbranched saturated C₁-C₈ alkyl. In embodiments, R¹ is unsubstituted unbranched saturated C₁-C₆ alkyl. In embodiments, R¹ is unsubstituted unbranched saturated C₁-C₄ alkyl. In embodiments, R¹ is unsubstituted unbranched saturated C₁-C₂ alkyl.

In embodiments, R¹ is unsubstituted unbranched unsaturated alkyl (e.g., C₁-C₂₅, C₁-C₂₀, C₁-C₁₂, C₁-C₈, C₁-C₆, C₁-C₄, or C₁-C₂). In embodiments, R¹ is unsubstituted unbranched unsaturated C₁-C₂₅ alkyl. In embodiments, R¹ is unsubstituted unbranched unsaturated C₁-C₂₀ alkyl. In embodiments, R¹ is unsubstituted unbranched unsaturated C₁-C₁₂ alkyl. In embodiments, R¹ is unsubstituted unbranched unsaturated C₁-C₈ alkyl. In embodiments, R¹ is unsubstituted unbranched unsaturated C₁-C₆ alkyl. In embodiments, R¹ is unsubstituted unbranched unsaturated C₁-C₄ alkyl. In embodiments, R¹ is unsubstituted unbranched unsaturated C₁-C₂ alkyl.

In embodiments, R¹ is unsubstituted C₉-C₁₉ alkyl. In embodiments, R¹ is unsubstituted branched C₉-C₁₉ alkyl. In embodiments, R¹ is unsubstituted unbranched C₉-C₁₉ alkyl. In embodiments, R¹ is unsubstituted branched saturated C₉-C₁₉ alkyl. In embodiments, R¹ is unsubstituted branched unsaturated C₉-C₁₉ alkyl. In embodiments, R¹ is unsubstituted unbranched saturated C₉-C₁₉ alkyl. In embodiments, R¹ is unsubstituted unbranched unsaturated C₉-C₁₉ alkyl.

In embodiments, R² is unsubstituted alkyl (e.g., C₁-C₂₅, C₁-C₂₀, C₁-C₁₂, C₁-C₈, C₁-C₈, C₁-C₄, or C₁-C₂). In embodiments, R² is unsubstituted C₁-C₂₅ alkyl. In embodiments, R² is unsubstituted C₁-C₂₀ alkyl. In embodiments, R² is unsubstituted C₁-C₁₂ alkyl. In embodiments, R² is unsubstituted C₁-C₈ alkyl. In embodiments, R² is unsubstituted C₁-C₆ alkyl. In embodiments, R² is unsubstituted C₁-C₄ alkyl. In embodiments, R² is unsubstituted C₁-C₂ alkyl.

In embodiments, R² is unsubstituted branched alkyl (e.g., C₁-C₂₅, C₁-C₂₀, C₁-C₁₂, C₁-C₈, C₁-C₆, C₁-C₄, or C₁-C₂). In embodiments, R² is unsubstituted branched C₁-C₂₅ alkyl. In embodiments, R² is unsubstituted branched C₁-C₂₀ alkyl. In embodiments, R² is unsubstituted branched C₁-C₁₂ alkyl. In embodiments, R² is unsubstituted branched C₁-C₈ alkyl. In embodiments, R² is unsubstituted branched C₁-C₆ alkyl. In embodiments, R² is unsubstituted branched C₁-C₄ alkyl. In embodiments, R² is unsubstituted branched C₁-C₂ alkyl.

In embodiments, R² is unsubstituted unbranched alkyl (e.g., C₁-C₂₅, C₁-C₂₀, C₁-C₁₂, C₁-C₈, C₁-C₆, C₁-C₄, or C₁-C₂). In embodiments, R² is unsubstituted unbranched C₁-C₂₅ alkyl. In embodiments, R² is unsubstituted unbranched C₁-C₂₀ alkyl. In embodiments, R² is unsubstituted unbranched C₁-C₁₂ alkyl. In embodiments, R² is unsubstituted unbranched C₁-C₈ alkyl. In embodiments, R² is unsubstituted unbranched C₁-C₆ alkyl. In embodiments, R² is unsubstituted unbranched C₁-C₄ alkyl. In embodiments, R² is unsubstituted unbranched C₁-C₂ alkyl.

In embodiments, R² is unsubstituted branched saturated alkyl (e.g., C₁-C₂₅, C₁-C₂₀, C₁-C₁₂, C₁-C₈, C₁-C₆, C₁-C₄, or C₁-C₂). In embodiments, R² is unsubstituted branched saturated C₁-C₂₅ alkyl. In embodiments, R² is unsubstituted branched saturated C₁-C₂₀ alkyl. In embodiments, R² is unsubstituted branched saturated C₁-C₁₂ alkyl. In embodiments, R² is unsubstituted branched saturated C₁-C₈ alkyl. In embodiments, R² is unsubstituted branched saturated C₁-C₆ alkyl. In embodiments, R² is unsubstituted branched saturated C₁-C₄ alkyl. In embodiments, R² is unsubstituted branched saturated C₁-C₂ alkyl.

In embodiments, R² is unsubstituted branched unsaturated alkyl (e.g., C₁-C₂₅, C₁-C₂₀, C₁-C₁₂, C₁-C₈, C₁-C₆, C₁-C₄, or C₁-C₂). In embodiments, R² is unsubstituted branched unsaturated C₁-C₂₅ alkyl. In embodiments, R² is unsubstituted branched unsaturated C₁-C₂₀ alkyl. In embodiments, R² is unsubstituted branched unsaturated C₁-C₁₂ alkyl. In embodiments, R² is unsubstituted branched unsaturated C₁-C₈ alkyl. In embodiments, R² is unsubstituted branched unsaturated C₁-C₆ alkyl. In embodiments, R² is unsubstituted branched unsaturated C₁-C₄ alkyl. In embodiments, R² is unsubstituted branched saturated C₁-C₂ alkyl.

In embodiments, R² is unsubstituted unbranched saturated alkyl (e.g., C₁-C₂₅, C₁-C₂₀, C₁-C₁₂, C₁-C₈, C₁-C₆, C₁-C₄, or C₁-C₂). In embodiments, R² is unsubstituted unbranched saturated C₁-C₂₅ alkyl. In embodiments, R² is unsubstituted unbranched saturated C₁-C₂₀ alkyl. In embodiments, R² is unsubstituted unbranched saturated C₁-C₁₂ alkyl. In embodiments, R² is unsubstituted unbranched saturated C₁-C₈ alkyl. In embodiments, R² is unsubstituted unbranched saturated C₁-C₆ alkyl. In embodiments, R² is unsubstituted unbranched saturated C₁-C₄ alkyl. In embodiments, R² is unsubstituted unbranched saturated C₁-C₂ alkyl.

In embodiments, R² is unsubstituted unbranched unsaturated alkyl (e.g., C₁-C₂₅, C₁-C₂₀, C₁-C₁₂, C₁-C₈, C₁-C₆, C₁-C₄, or C₁-C₂). In embodiments, R² is unsubstituted unbranched unsaturated C₁-C₂₅ alkyl. In embodiments, R² is unsubstituted unbranched unsaturated C₁-C₂₀ alkyl. In embodiments, R² is unsubstituted unbranched unsaturated C₁-C₁₂ alkyl. In embodiments, R² is unsubstituted unbranched unsaturated C₁-C₈ alkyl. In embodiments, R² is unsubstituted unbranched unsaturated C₁-C₆ alkyl. In embodiments, R² is unsubstituted unbranched unsaturated C₁-C₄ alkyl. In embodiments, R² is unsubstituted unbranched unsaturated C₁-C₂ alkyl.

In embodiments, R² is unsubstituted C₉-C₁₉ alkyl. In embodiments, R² is unsubstituted branched C₉-C₁₉ alkyl. In embodiments, R² is unsubstituted unbranched C₉-C₁₉ alkyl. In embodiments, R² is unsubstituted branched saturated C₉-C₁₉ alkyl. In embodiments, R² is unsubstituted branched unsaturated C₉-C₁₉ alkyl. In embodiments, R² is unsubstituted unbranched saturated C₉-C₁₉ alkyl. In embodiments, R² is unsubstituted unbranched unsaturated C₉-C₁₉ alkyl.

In embodiments, R³ is hydrogen, —NH₂, —OH, —SH, —C(O)H, —C(O)NH₂, —NHC(O)H, —NHC(O)OH, —NHC(O)NH₂, —C(O)OH, —OC(O)H, —N₃, substituted (e.g., substituted with a substituent group, a size-limited substituent group, or lower substituent group) or unsubstituted alkyl (e.g., C₁-C₂₀, C₁-C₁₂, C₁-C₈, C₁-C₆, C₁-C₄, or C₁-C₂), substituted (e.g., substituted with a substituent group, a size-limited substituent group, or lower substituent group) or unsubstituted heteroalkyl (e.g., 2 to 20 membered, 2 to 12 membered, 2 to 8 membered, 2 to 6 membered, 4 to 6 membered, 2 to 3 membered, or 4 to 5 membered), substituted (e.g., substituted with a substituent group, a size-limited substituent group, or lower substituent group) or unsubstituted cycloalkyl (e.g., C₃-C₁₀, C₃-C₈, C₃-C₆, C₄-C₆, or C₅-C₆), substituted (e.g., substituted with a substituent group, a size-limited substituent group, or lower substituent group) or unsubstituted heterocycloalkyl (e.g., 3 to 10 membered, 3 to 8 membered, 3 to 6 membered, 4 to 6 membered, 4 to 5 membered, or 5 to 6 membered), substituted (e.g., substituted with a substituent group, a size-limited substituent group, or lower substituent group) or unsubstituted aryl (e.g., C₆-C₁₂, C₆-C₁₀, or phenyl), or substituted (e.g., substituted with a substituent group, a size-limited substituent group, or lower substituent group) or unsubstituted heteroaryl (e.g., 5 to 12 membered, 5 to 10 membered, 5 to 9 membered, or 5 to 6 membered). In embodiments, R³ is hydrogen, —NH₂, —OH, —SH, —C(O)H, —C(O)NH₂, —NHC(O)H, —NHC(O)OH, —NHC(O)NH₂, —C(O)OH, —OC(O)H, —N₃, substituted (e.g., substituted with a substituent group, a size-limited substituent group, or lower substituent group) alkyl (e.g., C₁-C₂₀, C₁-C₁₂, C₁-C₈, C₁-C₆, C₁-C₄, or C₁-C₂), substituted (e.g., substituted with a substituent group, a size-limited substituent group, or lower substituent group) heteroalkyl (e.g., 2 to 20 membered, 2 to 12 membered, 2 to 8 membered, 2 to 6 membered, 4 to 6 membered, 2 to 3 membered, or 4 to 5 membered), substituted (e.g., substituted with a substituent group, a size-limited substituent group, or lower substituent group) cycloalkyl (e.g., C₃-C₁₀, C₃-C₈, C₃-C₆, C₄-C₆, or C₅-C₆), substituted (e.g., substituted with a substituent group, a size-limited substituent group, or lower substituent group) heterocycloalkyl (e.g., 3 to 10 membered, 3 to 8 membered, 3 to 6 membered, 4 to 6 membered, 4 to 5 membered, or 5 to 6 membered), substituted (e.g., substituted with a substituent group, a size-limited substituent group, or lower substituent group) aryl (e.g., C₆-C₁₂, C₆-C₁₀, or phenyl), or substituted (e.g., substituted with a substituent group, a size-limited substituent group, or lower substituent group) heteroaryl (e.g., 5 to 12 membered, 5 to 10 membered, 5 to 9 membered, or 5 to 6 membered). In embodiments, R³ is hydrogen, —NH₂, —OH, —SH, —C(O)H, —C(O)NH₂, —NHC(O)H, —NHC(O)OH, —NHC(O)NH₂, —C(O)OH, —OC(O)H, —N₃, unsubstituted alkyl (e.g., C₁-C₂₀, C₁-C₁₂, C₁-C₈, C₁-C₆, C₁-C₄, or C₁-C₂), unsubstituted heteroalkyl (e.g., 2 to 20 membered, 2 to 12 membered, 2 to 8 membered, 2 to 6 membered, 4 to 6 membered, 2 to 3 membered, or 4 to 5 membered), unsubstituted cycloalkyl (e.g., C₃-C₁₀, C₃-C₈, C₃-C₆, C₄-C₆, or C₅-C₆), unsubstituted heterocycloalkyl (e.g., 3 to 10 membered, 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). In embodiments, when R³ is substituted, R³ is substituted with a substituent group. In embodiments, when R³ is substituted, R³ is substituted with a size-limited substituent group. In embodiments, when R³ is substituted, R³ is substituted with a lower substituent group (e.g., oxo).

In embodiments, the uptake motif is represented by the structure:

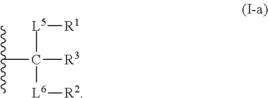

The uptake motif is attached to the remainder of the compounds provided here through the -L³-L⁴- moiety as set forth in Formula (I) above. The wavy line represents attachment to the L⁴ linker in Formula (I). R¹, R², R³, L⁵, and L⁶ in Formula (I-a) are as described in Formula (I), including embodiments thereof.

In embodiments, the compound comprises one or more uptake motifs having a structure shown in Table 2 below. In embodiments, the compound comprises a DTx-01-01 motif in Table 2. In embodiments, the compound comprises a DTx-01-03 motif 1 of Table 2. In embodiments, the compound comprises a DTx-01-06 motif in Table 2. In embodiments, the compound comprises a DTx-01-08 motif in Table 2. In embodiments, the compound comprises a DTx-01-11 motif in Table 2. In embodiments, the compound comprises a DTx-01-13 motif in Table 2. In embodiments, the compound comprises a DTx-01-30 motif in Table 2. In embodiments, the compound comprises a DTx-01-31 motif in Table 2. In embodiments, the compound comprises a DTx-01-32 motif in Table 2. In embodiments, the compound comprises a DTx-01-33 motif in Table 2. In embodiments, the compound comprises a DTx-01-34 motif in Table 2. In embodiments, the compound comprises a DTx-01-35 motif in Table 2. In embodiments, the compound comprises a DTx-01-36 motif in Table 2. In embodiments, the compound comprises a DTx-01-39 motif in Table 2. In embodiments, the compound comprises a DTx-01-43 motif in Table 2. In embodiments, the compound comprises a DTx-01-44 motif in Table 2. In embodiments, the compound comprises a DTx-01-45 motif in Table 2. In embodiments, the compound comprises a DTx-01-46 motif in Table 2. In embodiments, the compound comprises a DTx-01-50 motif in Table 2. In embodiments, the compound comprises a DTx-01-51 motif in Table 2. In embodiments, the compound comprises a DTx-01-52 motif in Table 2. In embodiments, the compound comprises a DTx-01-53 motif in Table 2. In embodiments, the compound comprises a DTx-01-54 motif in Table 2. In embodiments, the compound comprises a DTx-01-55 motif in Table 2. In embodiments, the compound comprises a DTx-03-06 motif in Table 2. In embodiments, the compound comprises a DTx-03-50 motif in Table 2. In embodiments, the compound comprises a DTx-03-51 motif in Table 2. In embodiments, the compound comprises a DTx-03-52 motif in Table 2. In embodiments, the compound comprises a DTx-03-53 motif in Table 2. In embodiments, the compound comprises a DTx-03-54 motif in Table 2. In embodiments, the compound comprises a DTx-03-55 motif in Table 2. In embodiments, the compound comprises a DTx-04-01 motif in Table 2. In embodiments, the compound comprises a DTx-05-01 motif in Table 2. In embodiments, the compound comprises a DTx-06-06 motif in Table 2. In embodiments, the compound comprises a DTx-06-50 motif in Table 2. In embodiments, the compound comprises a DTx-06-51 motif in Table 2. In embodiments, the compound comprises a DTx-06-52 motif in Table 2. In embodiments, the compound comprises a DTx-06-53 motif in Table 2. In embodiments, the compound comprises a DTx-06-54 motif in Table 2. In embodiments, the compound comprises a DTx-06-55 motif in Table 2. In embodiments, the compound comprises a DTx-08-01 motif in Table 2. In embodiments, the compound comprises a DTx-09-01 motif in Table 2. In embodiments, the compound comprises a DTx-10-01 motif in Table 2. In embodiments, the compound comprises a DTx-11-01 motif in Table 2. In embodiments, the compound comprises a DTx-01-60 motif in Table 2. In embodiments, the compound comprises a DTx-01-61 motif in Table 2. In embodiments, the compound comprises a DTx-01-62 motif in Table 2. In embodiments, the compound comprises a DTx-01-63 motif in Table 2. In embodiments, the compound comprises a DTx-01-64 motif in Table 2. In embodiments, the compound comprises a DTx-01-65 motif in Table 2. In embodiments, the compound comprises a DTx-01-66 motif in Table 2. In embodiments, the compound comprises a DTx-01-67 motif in Table 2. In embodiments, the compound comprises a DTx-01-68 motif in Table 2. In embodiments, the compound comprises a DTx-01-69 motif in Table 2. In embodiments, the compound comprises a DTx-01-70 motif in Table 2. In embodiments, the compound comprises a DTx-01-71 motif in Table 2. In embodiments, the compound comprises a DTx-01-72 motif in Table 2. In embodiments, the compound comprises a DTx-01-73 motif in Table 2. In embodiments, the compound comprises a DTx-01-74 motif in Table 2. In embodiments, the compound comprises a DTx-01-75 motif in Table 2. In embodiments, the compound comprises a DTx-01-76 motif in Table 2. In embodiments, the compound comprises a DTx-01-77 motif in Table 2. In embodiments, the compound comprises a DTx-01-78 motif in Table 2. In embodiments, the compound comprises a DTx-01-79 motif in Table 2. In embodiments, the compound comprises a DTx-01-80 motif in Table 2. In embodiments, the compound comprises a DTx-01-81 motif in Table 2. In embodiments, the compound comprises a DTx-01-82 motif in Table 2. In embodiments, the compound comprises a DTx-1-83 motif in Table 2. In embodiments, the compound comprises a DTx-01-84 motif in Table 2. In embodiments, the compound comprises a DTx-01-85 motif in Table 2. In embodiments, the compound comprises a DTx-01-86 motif in Table 2. In embodiments, the compound comprises a DTx-01-87 motif in Table 2. In embodiments, the compound comprises a DTx-01-88 motif in Table 2. In embodiments, the compound comprises a DTx-01-89 motif in Table 2. In embodiments, the compound comprises a DTx-01-90 motif in Table 2. In embodiments, the compound comprises a DTx-01-91 motif in Table 2. In embodiments, the compound comprises a DTx-01-92 motif in Table 2. In embodiments, the compound comprises a DTx-01-93 motif in Table 2. In embodiments, the compound comprises a DTx-01-94 motif in Table 2. In embodiments, the compound comprises a DTx-01-95 motif in Table 2. In embodiments, the compound comprises a DTx-01-96 motif in Table 2. In embodiments, the compound comprises a DTx-01-97 motif in Table 2. In embodiments, the compound comprises a DTx-01-98 motif in Table 2. In embodiments, the compound comprises a DTx-01-99 motif in Table 2. In embodiments, the compound comprises a DTx-01-100 motif in Table 2. In embodiments, the compound comprises a DTx-01-101 motif in Table 2.

TABLE 2
Uptake Motif

| Uptake | |
|---|---|
| Motif | |
| Name | Uptake Motif Structure |
| DTx-01-01 | 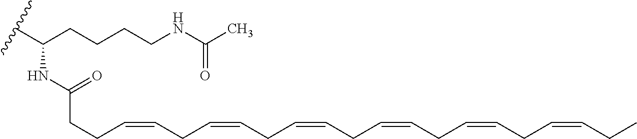 [SMILES: *[C@H](CCCCNC(C)=O)NC(=O)CC/C=C\C/C=C\C/C=C\C/C=C\C/C=C\C/C=C\CC] |
| DTx-01-03 | 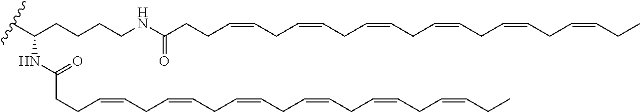 [SMILES: *[C@H](CCCCNC(=O)CC/C=C\C/C=C\C/C=C\C/C=C\C/C=C\C/C=C\CC)NC(=O)CC/C=C\C/C=C\C/C=C\C/C=C\C/C=C\C/C=C\CC] |
| DTx-01-06 | 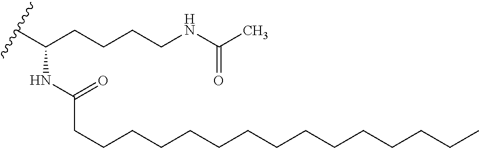 [SMILES: *[C@H](CCCCNC(C)=O)NC(=O)CCCCCCCCCCCCCCC] |
| DTx-01-07 | 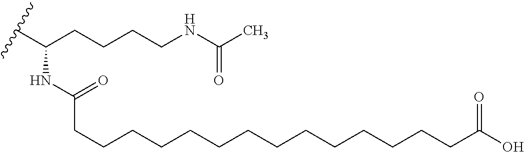 [SMILES: *[C@H](CCCCNC(C)=O)NC(=O)CCCCCCCCCCCCCCC(=O)O] |
| DTx-01-08 | 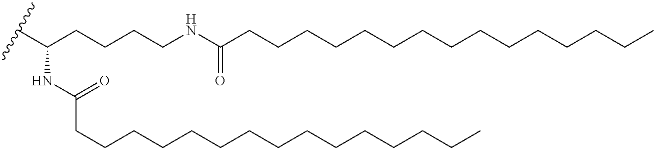 [SMILES: *[C@H](CCCCNC(=O)CCCCCCCCCCCCCCC)NC(=O)CCCCCCCCCCCCCCC] |
| DTx-01-09 | 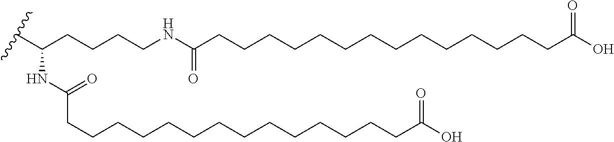 [SMILES: *[C@H](CCCCNC(=O)CCCCCCCCCCCCCCC(=O)O)NC(=O)CCCCCCCCCCCCCCC(=O)O] |
| DTx-01-11 | 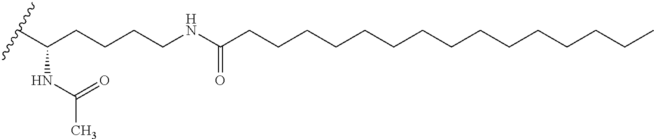 [SMILES: *[C@H](CCCCNC(=O)CCCCCCCCCCCCCCC)NC(C)=O] |
| DTx-01-12 | 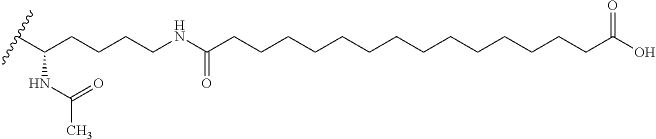 [SMILES: *[C@H](CCCCNC(=O)CCCCCCCCCCCCCCC(=O)O)NC(C)=O] |
| DTx-01-13 | 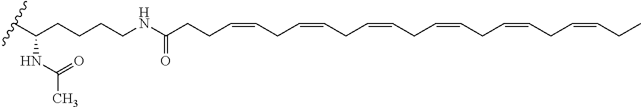 [SMILES: *[C@H](CCCCNC(=O)CC/C=C\C/C=C\C/C=C\C/C=C\C/C=C\C/C=C\CC)NC(C)=O] |
| DTx-01-30 | 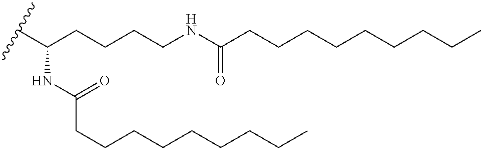 [SMILES: *[C@H](CCCCNC(=O)CCCCCCCCC)NC(=O)CCCCCCCCC] |
| DTx-01-31 | 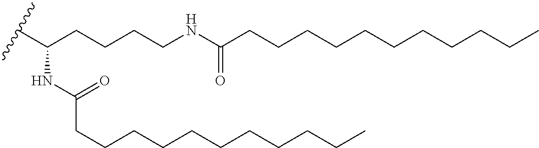 [SMILES: *[C@H](CCCCNC(=O)CCCCCCCCCCC)NC(=O)CCCCCCCCCCC] |
| DTx-01-32 | 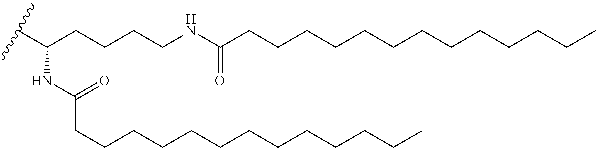 [SMILES: *[C@H](CCCCNC(=O)CCCCCCCCCCCCC)NC(=O)CCCCCCCCCCCCC] |
| DTx-01-33 | 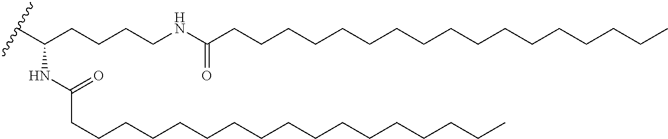 [SMILES: *[C@H](CCCCNC(=O)CCCCCCCCCCCCCCCCC)NC(=O)CCCCCCCCCCCCCCCCC] |
| DTx-01-34 | 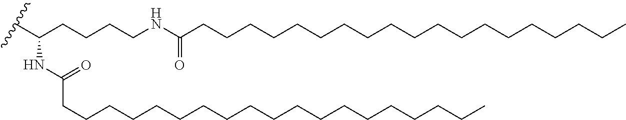 [SMILES: *[C@H](CCCCNC(=O)CCCCCCCCCCCCCCCCCCC)NC(=O)CCCCCCCCCCCCCCCCCCC] |
| DTx-01-35 | 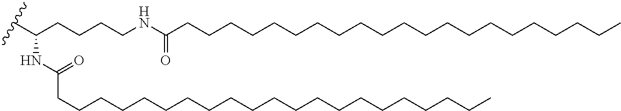 [SMILES: *[C@H](CCCCNC(=O)CCCCCCCCCCCCCCCCCCCCC)NC(=O)CCCCCCCCCCCCCCCCCCCCC] |
| DTx-01-36 | 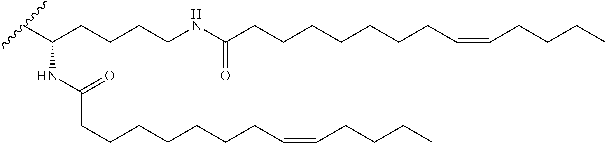 [SMILES: *[C@H](CCCCNC(=O)CCCCCCC/C=C\CCCC)NC(=O)CCCCCCC/C=C\CCCC] |
| DTx-01-39 | 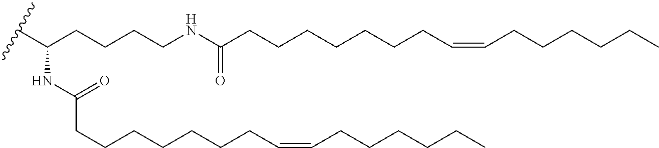 [SMILES: *[C@H](CCCCNC(=O)CCCCCCC/C=C\CCCCCC)NC(=O)CCCCCCC/C=C\CCCCCC] |
| DTx-01-43 | 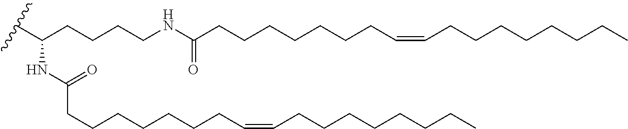 [SMILES: *[C@H](CCCCNC(=O)CCCCCCC/C=C\CCCCCCCC)NC(=O)CCCCCCC/C=C\CCCCCCCC] |
| DTx-01-44 | 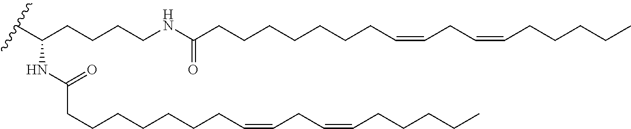 [SMILES: *[C@H](CCCCNC(=O)CCCCCCC/C=C\C/C=C\CCCCC)NC(=O)CCCCCCC/C=C\C/C=C\CCCCC] |
| DTx-01-45 | 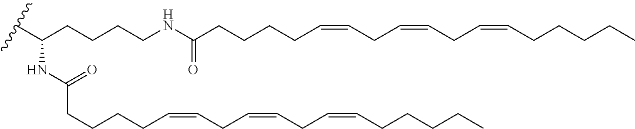 [SMILES: *[C@H](CCCCNC(=O)CCCC/C=C\C/C=C\C/C=C\CCCCC)NC(=O)CCCC/C=C\C/C=C\C/C=C\CCCCC] |
| DTx-01-46 | 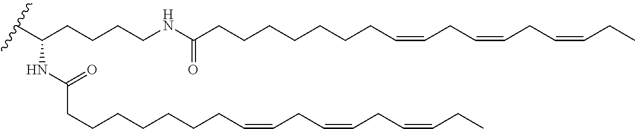 [SMILES: *[C@H](CCCCNC(=O)CCCCCCC/C=C\C/C=C\C/C=C\CC)NC(=O)CCCCCCC/C=C\C/C=C\C/C=C\CC] |
| DTx-01-50 | 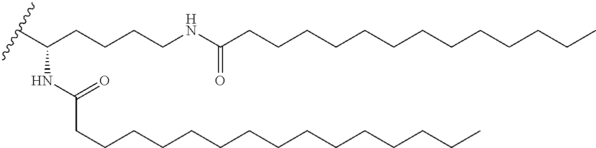 [SMILES: *[C@H](CCCCNC(=O)CCCCCCCCCCCCC)NC(=O)CCCCCCCCCCCCCCC] |
| DTx-01-51 | 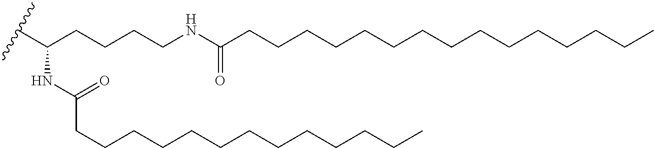 [SMILES: *[C@H](CCCCNC(=O)CCCCCCCCCCCCCCC)NC(=O)CCCCCCCCCCCCC] |
| DTx-01-52 | 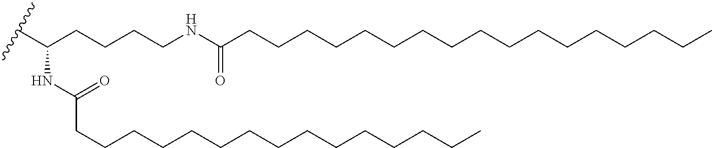 [SMILES: *[C@H](CCCCNC(=O)CCCCCCCCCCCCCCCCC)NC(=O)CCCCCCCCCCCCCCC] |
| DTx-01-53 | 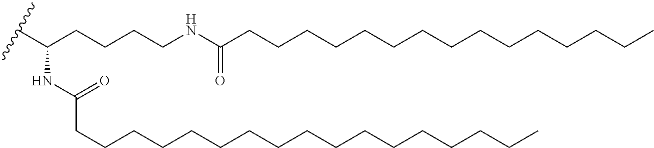 [SMILES: *[C@H](CCCCNC(=O)CCCCCCCCCCCCCCC)NC(=O)CCCCCCCCCCCCCCCCC] |
| DTx-01-54 | 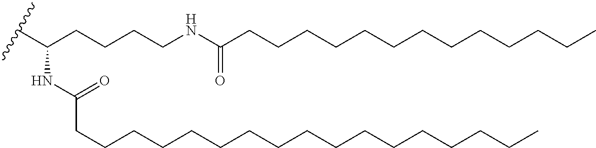 [SMILES: *[C@H](CCCCNC(=O)CCCCCCCCCCCCC)NC(=O)CCCCCCCCCCCCCCCCC] |
| DTx-01-55 | 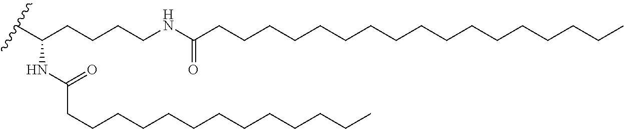 [SMILES: *[C@H](CCCCNC(=O)CCCCCCCCCCCCCCCCC)NC(=O)CCCCCCCCCCCCC] |
| DTx-03-06 | 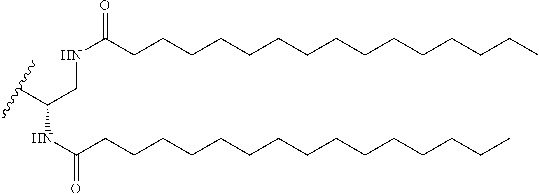 [SMILES: *[C@H](CNC(=O)CCCCCCCCCCCCCCC)NC(=O)CCCCCCCCCCCCCCC] |
| DTx-03-50 | 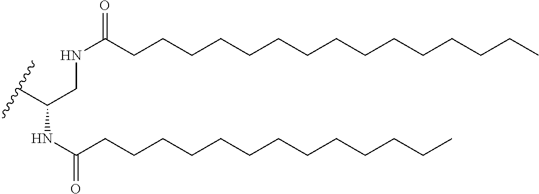 [SMILES: *[C@H](CNC(=O)CCCCCCCCCCCCCCC)NC(=O)CCCCCCCCCCCCC] |
| DTx-03-51 | 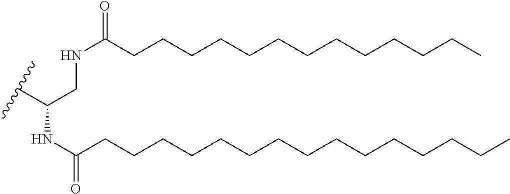 [SMILES: *[C@H](CNC(=O)CCCCCCCCCCCCC)NC(=O)CCCCCCCCCCCCCCC] |
| DTx-03-52 | 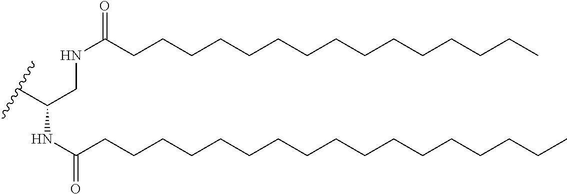 [SMILES: *[C@H](CNC(=O)CCCCCCCCCCCCCCC)NC(=O)CCCCCCCCCCCCCCCCC] |
| DTx-03-53 | 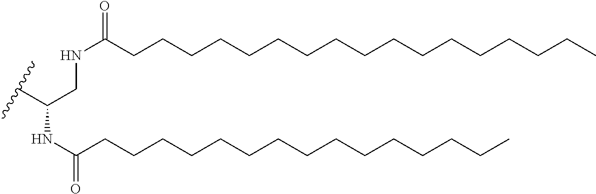 [SMILES: *[C@H](CNC(=O)CCCCCCCCCCCCCCCCC)NC(=O)CCCCCCCCCCCCCCC] |
| DTx-03-54 | 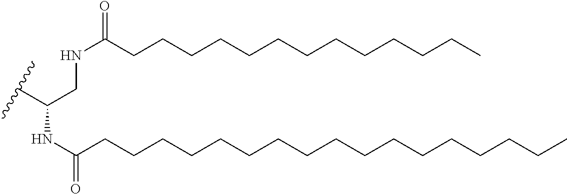 [SMILES: *[C@H](CNC(=O)CCCCCCCCCCCCC)NC(=O)CCCCCCCCCCCCCCCCC] |
| DTx-03-55 | 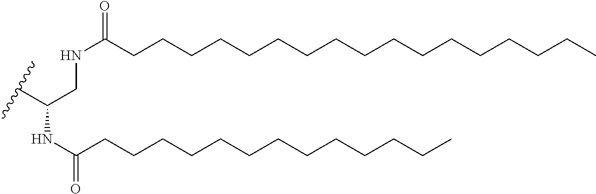 [SMILES: *[C@H](CNC(=O)CCCCCCCCCCCCCCCCC)NC(=O)CCCCCCCCCCCCC] |
| DTx-04-01 | 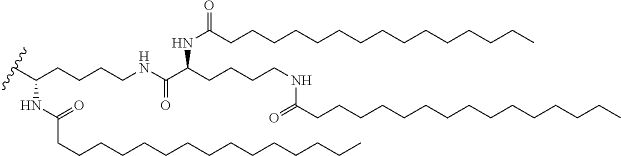 [SMILES: *[C@H](CCCCNC(=O)[C@H](CCCCNC(=O)CCCCCCCCCCCCCCC)NC(=O)CCCCCCCCCCCCCCC)NC(=O)CCCCCCCCCCCCCCC] |
| DTx-05-01 | 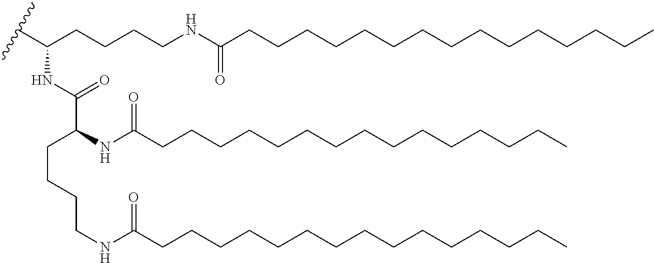 [SMILES: *[C@H](CCCCNC(=O)CCCCCCCCCCCCCCC)NC(=O)[C@H](CCCCNC(=O)CCCCCCCCCCCCCCC)NC(=O)CCCCCCCCCCCCCCC] |
| DTx-06-06 | 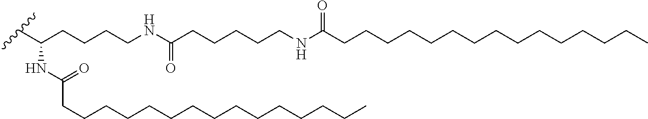 [SMILES: *[C@H](CCCCNC(=O)CCCCCNC(=O)CCCCCCCCCCCCCCC)NC(=O)CCCCCCCCCCCCCCC] |
| DTx-06-50 | 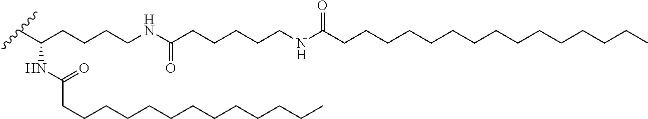 [SMILES: *[C@H](CCCCNC(=O)CCCCCNC(=O)CCCCCCCCCCCCCCC)NC(=O)CCCCCCCCCCCCC] |
| DTx-06-51 | 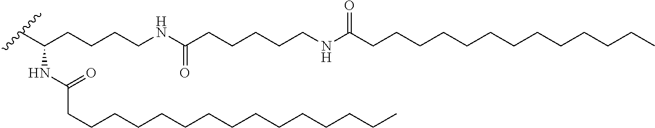 [SMILES: *[C@H](CCCCNC(=O)CCCCCNC(=O)CCCCCCCCCCCCC)NC(=O)CCCCCCCCCCCCCCC] |
| DTx-06-52 | 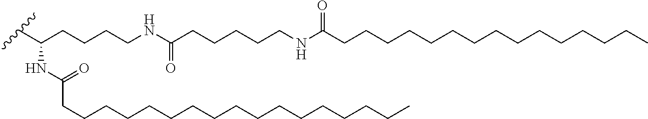 [SMILES: *[C@H](CCCCNC(=O)CCCCCNC(=O)CCCCCCCCCCCCCCC)NC(=O)CCCCCCCCCCCCCCCCC] |
| DTx-06-53 | 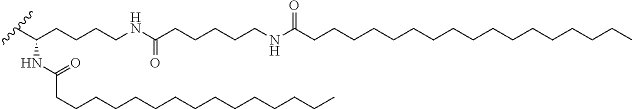 [SMILES: *[C@H](CCCCNC(=O)CCCCCNC(=O)CCCCCCCCCCCCCCCCC)NC(=O)CCCCCCCCCCCCCCC] |
| DTx-06-54 | 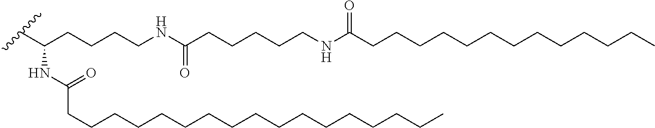 [SMILES: *[C@H](CCCCNC(=O)CCCCCNC(=O)CCCCCCCCCCCCC)NC(=O)CCCCCCCCCCCCCCCCC] |
| DTx-06-55 | 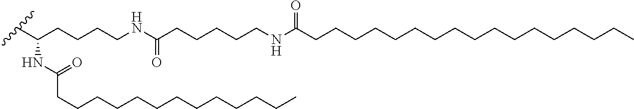 [SMILES: *[C@H](CCCCNC(=O)CCCCCNC(=O)CCCCCCCCCCCCCCCCC)NC(=O)CCCCCCCCCCCCC] |
| DTx-08-01 | 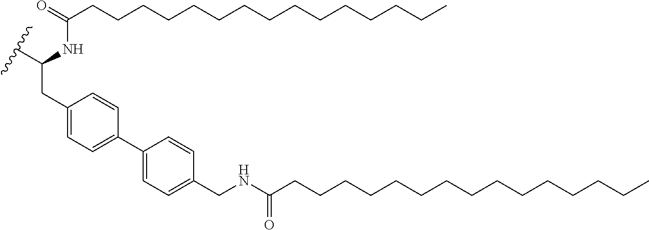 [SMILES: *[C@H](Cc1ccc(-c2ccc(CNC(=O)CCCCCCCCCCCCCCC)cc2)cc1)NC(=O)CCCCCCCCCCCCCCC] |
| DTx-09-01 | 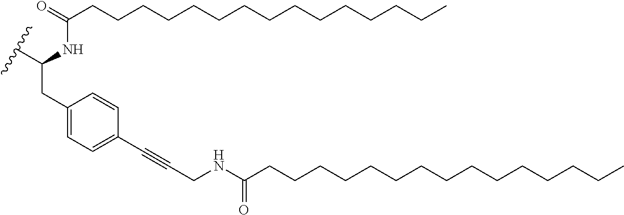 [SMILES: *[C@H](Cc1ccc(C#CCNC(=O)CCCCCCCCCCCCCCC)cc1)NC(=O)CCCCCCCCCCCCCCC] |
| DTx-10-01 | 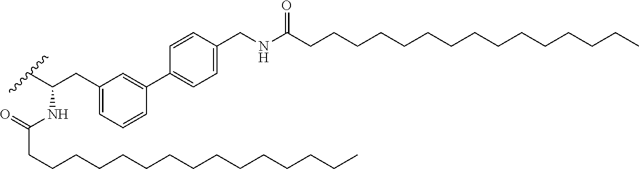 [SMILES: *[C@H](Cc1cccc(-c2ccc(CNC(=O)CCCCCCCCCCCCCCC)cc2)c1)NC(=O)CCCCCCCCCCCCCCC] |
| DTx-11-01 | 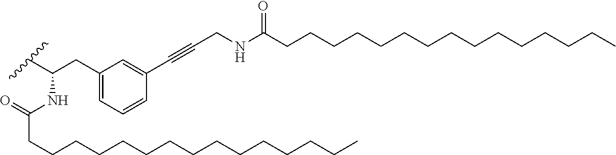 [SMILES: *[C@H](Cc1cccc(C#CCNC(=O)CCCCCCCCCCCCCCC)c1)NC(=O)CCCCCCCCCCCCCCC] |
| DTx-01-60 | 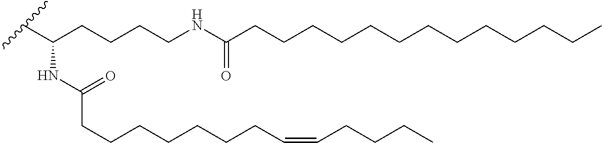 [SMILES: *[C@H](CCCCNC(=O)CCCCCCCCCCCCC)NC(=O)CCCCCCC/C=C\CCCC] |
| DTx-01-61 | 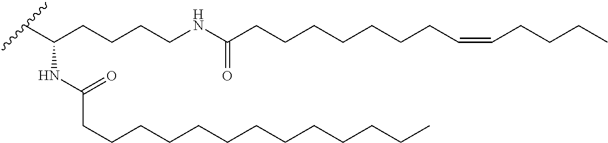 [SMILES: *[C@H](CCCCNC(=O)CCCCCCC/C=C\CCCC)NC(=O)CCCCCCCCCCCCC] |
| DTx-01-62 | 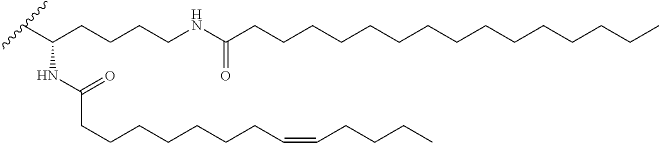 [SMILES: *[C@H](CCCCNC(=O)CCCCCCCCCCCCCCC)NC(=O)CCCCCCC/C=C\CCCC] |
| DTx-01-63 | 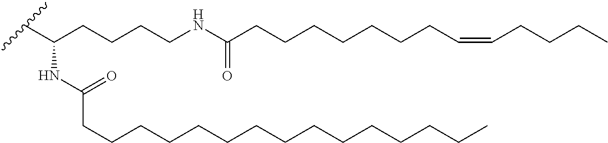 [SMILES: *[C@H](CCCCNC(=O)CCCCCCC/C=C\CCCC)NC(=O)CCCCCCCCCCCCCCC] |
| DTx-01-64 | 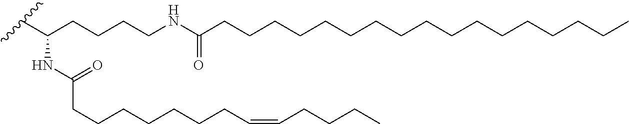 [SMILES: *[C@H](CCCCNC(=O)CCCCCCCCCCCCCCCCC)NC(=O)CCCCCCC/C=C\CCCC] |
| DTx-01-65 | 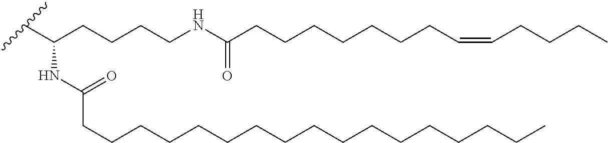 [SMILES: *[C@H](CCCCNC(=O)CCCCCCC/C=C\CCCC)NC(=O)CCCCCCCCCCCCCCCCC] |
| DTx-01-66 | 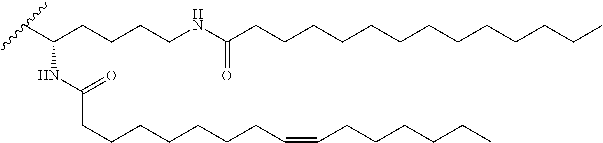 [SMILES: *[C@H](CCCCNC(=O)CCCCCCCCCCCCC)NC(=O)CCCCCCC/C=C\CCCCCC] |
| DTx-01-67 | 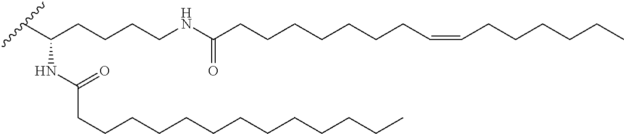 [SMILES: *[C@H](CCCCNC(=O)CCCCCCC/C=C\CCCCCC)NC(=O)CCCCCCCCCCCCC] |
| DTx-01-68 | 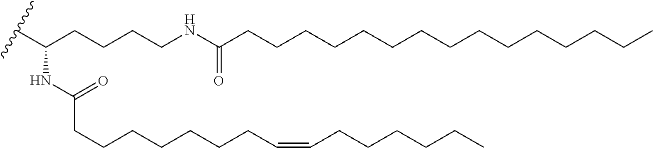 [SMILES: *[C@H](CCCCNC(=O)CCCCCCCCCCCCCCC)NC(=O)CCCCCCC/C=C\CCCCCC] |
| DTx-01-69 | 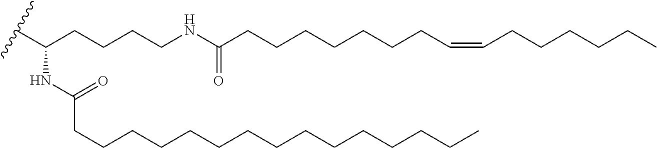 [SMILES: *[C@H](CCCCNC(=O)CCCCCCC/C=C\CCCCCC)NC(=O)CCCCCCCCCCCCCCC] |
| DTx-01-70 | 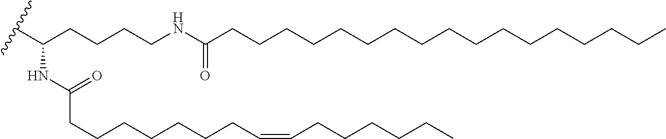 [SMILES: *[C@H](CCCCNC(=O)CCCCCCCCCCCCCCCCC)NC(=O)CCCCCCC/C=C\CCCCCC] |
| DTx-01-71 | 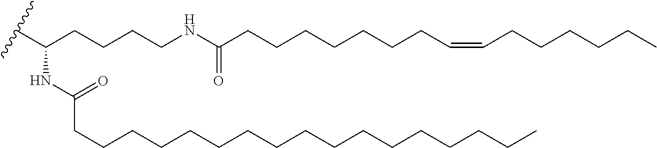 [SMILES: *[C@H](CCCCNC(=O)CCCCCCC/C=C\CCCCCC)NC(=O)CCCCCCCCCCCCCCCCC] |
| DTx-01-72 | 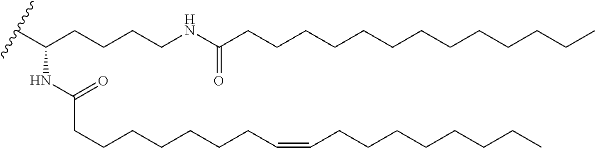 [SMILES: *[C@H](CCCCNC(=O)CCCCCCCCCCCCC)NC(=O)CCCCCCC/C=C\CCCCCCCC] |
| DTx-01-73 | 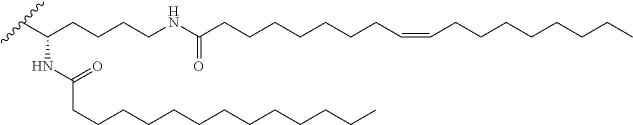 [SMILES: *[C@H](CCCCNC(=O)CCCCCCC/C=C\CCCCCCCC)NC(=O)CCCCCCCCCCCCC] |
| DTx-01-74 | 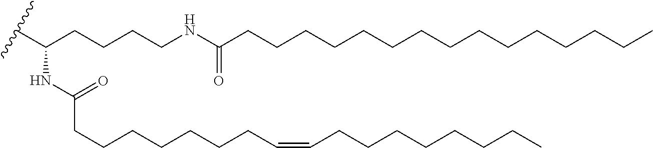 [SMILES: *[C@H](CCCCNC(=O)CCCCCCCCCCCCCCC)NC(=O)CCCCCCC/C=C\CCCCCCCC] |
| DTx-01-75 | 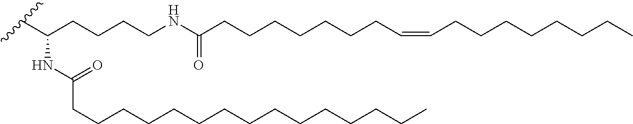 [SMILES: *[C@H](CCCCNC(=O)CCCCCCC/C=C\CCCCCCCC)NC(=O)CCCCCCCCCCCCCCC] |
| DTx-01-76 | 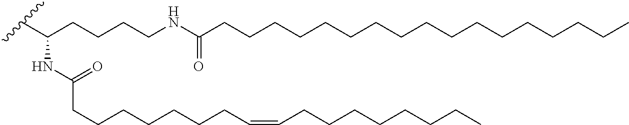 [SMILES: *[C@H](CCCCNC(=O)CCCCCCCCCCCCCCCCC)NC(=O)CCCCCCC/C=C\CCCCCCCC] |
| DTx-01-77 | 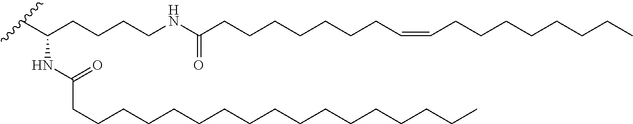 [SMILES: *[C@H](CCCCNC(=O)CCCCCCC/C=C\CCCCCCCC)NC(=O)CCCCCCCCCCCCCCCCC] |
| DTx-01-78 | 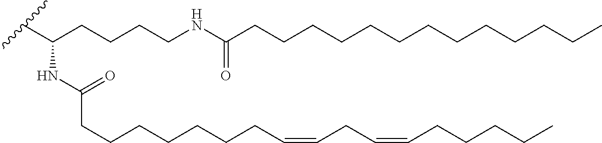 [SMILES: *[C@H](CCCCNC(=O)CCCCCCCCCCCCC)NC(=O)CCCCCCC/C=C\C/C=C\CCCCC] |
| DTx-01-79 | 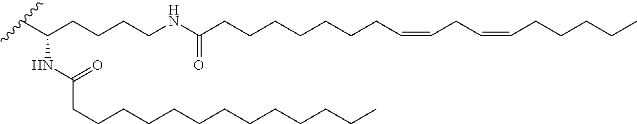 [SMILES: *[C@H](CCCCNC(=O)CCCCCCC/C=C\C/C=C\CCCCC)NC(=O)CCCCCCCCCCCCC] |
| DTx-01-80 | 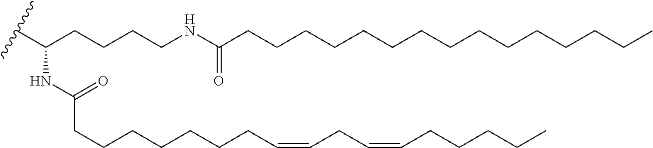 [SMILES: *[C@H](CCCCNC(=O)CCCCCCCCCCCCCCC)NC(=O)CCCCCCC/C=C\C/C=C\CCCCC] |
| DTx-01-81 | 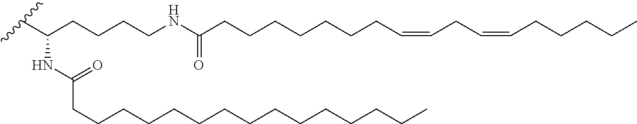 [SMILES: *[C@H](CCCCNC(=O)CCCCCCC/C=C\C/C=C\CCCCC)NC(=O)CCCCCCCCCCCCCCC] |
| DTx-01-82 | 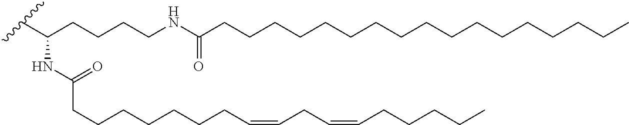 [SMILES: *[C@H](CCCCNC(=O)CCCCCCCCCCCCCCCCC)NC(=O)CCCCCCC/C=C\C/C=C\CCCCC] |
| DTx-01-83 | 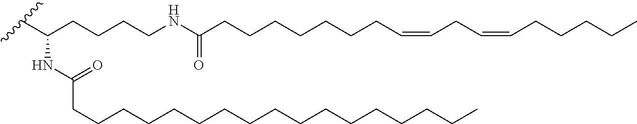 [SMILES: *[C@H](CCCCNC(=O)CCCCCCC/C=C\C/C=C\CCCCC)NC(=O)CCCCCCCCCCCCCCCCC] |
| DTx-01-84 | 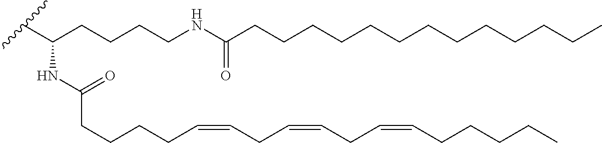 [SMILES: *[C@H](CCCCNC(=O)CCCCCCCCCCCCC)NC(=O)CCCC/C=C\C/C=C\C/C=C\CCCCC] |
| DTx-01-85 | 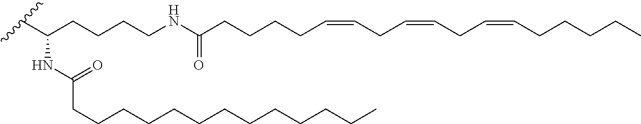 [SMILES: *[C@H](CCCCNC(=O)CCCC/C=C\C/C=C\C/C=C\CCCCC)NC(=O)CCCCCCCCCCCCC] |
| DTx-01-86 | 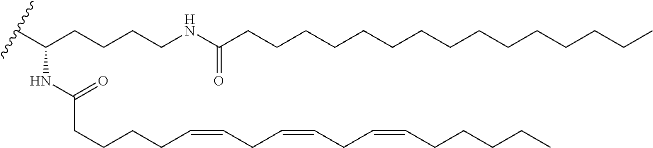 [SMILES: *[C@H](CCCCNC(=O)CCCCCCCCCCCCCCC)NC(=O)CCCC/C=C\C/C=C\C/C=C\CCCCC] |
| DTx-01-87 | 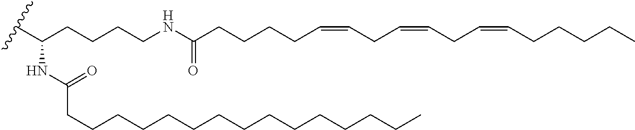 [SMILES: *[C@H](CCCCNC(=O)CCCC/C=C\C/C=C\C/C=C\CCCCC)NC(=O)CCCCCCCCCCCCCCC] |
| DTx-01-88 | 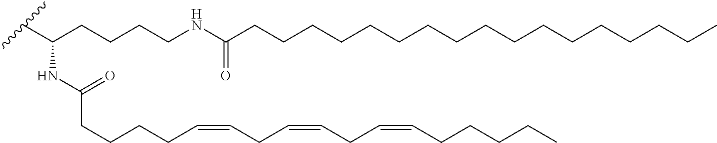 [SMILES: *[C@H](CCCCNC(=O)CCCCCCCCCCCCCCCCC)NC(=O)CCCC/C=C\C/C=C\C/C=C\CCCCC] |
| DTx-01-89 | 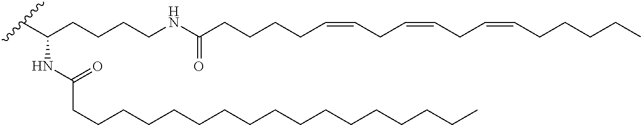 [SMILES: *[C@H](CCCCNC(=O)CCCC/C=C\C/C=C\C/C=C\CCCCC)NC(=O)CCCCCCCCCCCCCCCCC] |
| DTx-01-90 | 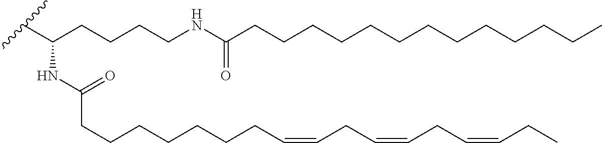 [SMILES: *[C@H](CCCCNC(=O)CCCCCCCCCCCCC)NC(=O)CCCCCCC/C=C\C/C=C\C/C=C\CC] |
| DTx-01-91 | 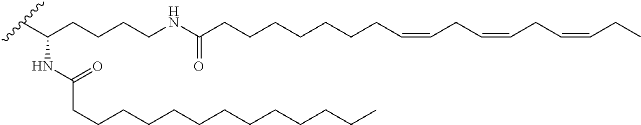 [SMILES: *[C@H](CCCCNC(=O)CCCCCCC/C=C\C/C=C\C/C=C\CC)NC(=O)CCCCCCCCCCCCC] |
| DTx-01-92 | 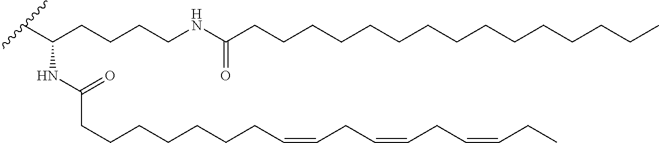 [SMILES: *[C@H](CCCCNC(=O)CCCCCCCCCCCCCCC)NC(=O)CCCCCCC/C=C\C/C=C\C/C=C\CC] |
| DTx-01-93 | 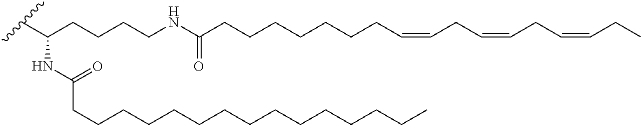 [SMILES: *[C@H](CCCCNC(=O)CCCCCCC/C=C\C/C=C\C/C=C\CC)NC(=O)CCCCCCCCCCCCCCC] |
| DTx-01-94 | 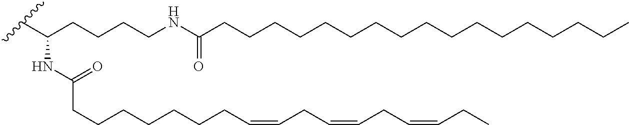 [SMILES: *[C@H](CCCCNC(=O)CCCCCCCCCCCCCCCCC)NC(=O)CCCCCCC/C=C\C/C=C\C/C=C\CC] |
| DTx-01-95 | 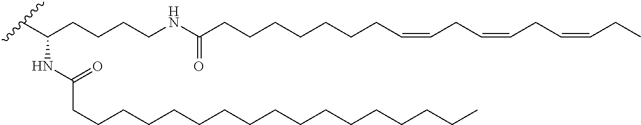 [SMILES: *[C@H](CCCCNC(=O)CCCCCCC/C=C\C/C=C\C/C=C\CC)NC(=O)CCCCCCCCCCCCCCCCC] |
| DTx-01-96 | 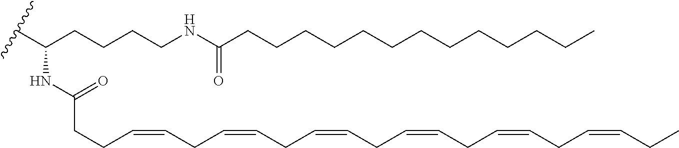 [SMILES: *[C@H](CCCCNC(=O)CCCCCCCCCCCCC)NC(=O)CC/C=C\C/C=C\C/C=C\C/C=C\C/C=C\C/C=C\CC] |
| DTx-01-97 | 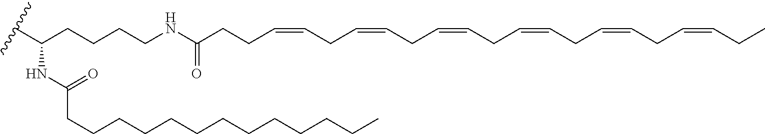 [SMILES: *[C@H](CCCCNC(=O)CC/C=C\C/C=C\C/C=C\C/C=C\C/C=C\C/C=C\CC)NC(=O)CCCCCCCCCCCCC] |
| DTx-01-98 | 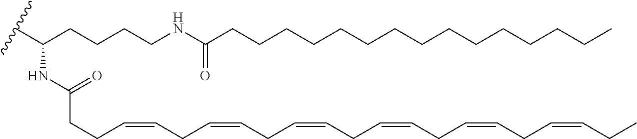 [SMILES: *[C@H](CCCCNC(=O)CCCCCCCCCCCCCCC)NC(=O)CC/C=C\C/C=C\C/C=C\C/C=C\C/C=C\C/C=C\CC] |
| DTx-01-99 | 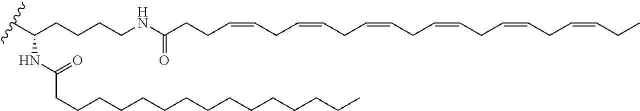 [SMILES: *[C@H](CCCCNC(=O)CC/C=C\C/C=C\C/C=C\C/C=C\C/C=C\C/C=C\CC)NC(=O)CCCCCCCCCCCCCCC] |
| DTx-01-100 | 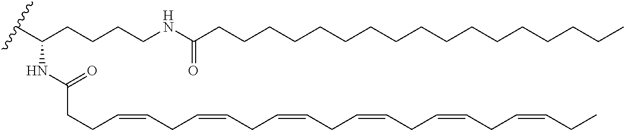 [SMILES: *[C@H](CCCCNC(=O)CCCCCCCCCCCCCCCCC)NC(=O)CC/C=C\C/C=C\C/C=C\C/C=C\C/C=C\C/C=C\CC] |
| DTx-01- +101v2 | 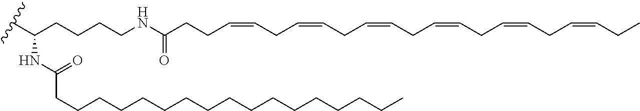 [SMILES: *[C@H](CCCCNC(=O)CC/C=C\C/C=C\C/C=C\C/C=C\C/C=C\C/C=C\CC)NC(=O)CCCCCCCCCCCCCCCCC] |

In embodiments, DTx-01-01 is attached to the double-stranded nucleic acid (A) through -L³-L⁴-, wherein -L³-L⁴- is

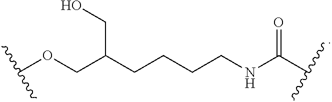 [SMILES: *OCC(CO)CCCCNC(*)=O]

In embodiments, DTx-01-03 is attached to the double-stranded nucleic acid (A) through -L³-L⁴-, wherein -L³-L⁴- is

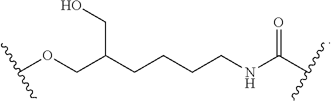 [SMILES: *OCC(CO)CCCCNC(*)=O]

In embodiments, DTx-01-06 is attached to the double-stranded nucleic acid (A) through -L³-L⁴- wherein -L³-L⁴- is

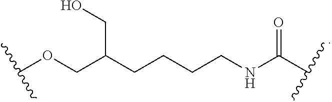 [SMILES: *OCC(CO)CCCCNC(*)=O]

In embodiments, DTx-01-08 is attached to the double-stranded nucleic acid (A) through -L³-L⁴-, wherein -L³-L⁴- is

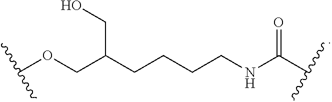 [SMILES: *OCC(CO)CCCCNC(*)=O]

In embodiments, DTx-01-11 is attached to the double-stranded nucleic acid (A) through -L³-L⁴-, wherein -L³-L⁴- is

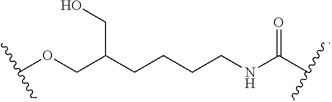 [SMILES: *OCC(CO)CCCCNC(*)=O]

In embodiments, DTx-01-13 is attached to the double-stranded nucleic acid (A) through -L³-L⁴-, wherein -L³-L⁴- is

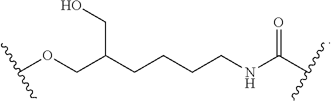 [SMILES: *OCC(CO)CCCCNC(*)=O]

In embodiments, DTx-01-30 is attached to the double-stranded nucleic acid (A) through -L³-L⁴-, wherein -L³-L⁴- is

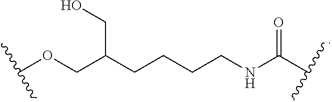 [SMILES: *OCC(CO)CCCCNC(*)=O]

In embodiments, DTx-01-31 is attached to the double-stranded nucleic acid (A) through -L³-L⁴-, wherein -L³-L⁴- is

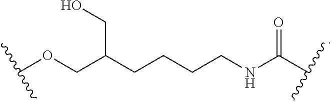 [SMILES: *OCC(CO)CCCCNC(*)=O]

In embodiments, DTx-01-32 is attached to the double-stranded nucleic acid (A) through -L³-L⁴-, wherein -L³-L⁴- is

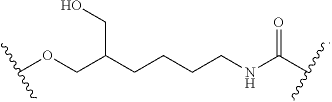 [SMILES: *OCC(CO)CCCCNC(*)=O]

In embodiments, DTx-01-33 is attached to the double-stranded nucleic acid (A) through -L³-L⁴-, wherein -L³-L⁴- is

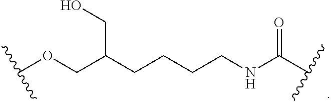 [SMILES: *OCC(CO)CCCCNC(*)=O]

In embodiments, DTx-01-34 is attached to the double-stranded nucleic acid (A) through -L³-L⁴-, wherein -L³-L⁴- is

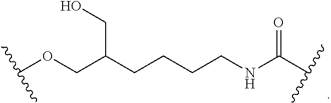 [SMILES: *OCC(CO)CCCCNC(*)=O]

In embodiments, DTx-01-35 is attached to the double-stranded nucleic acid (A) through -L³-L⁴-, wherein -L³-L⁴- is

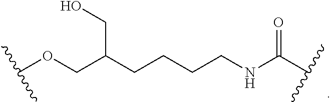 [SMILES: *OCC(CO)CCCCNC(*)=O]

In embodiments, DTx-01-36 is attached to the double-stranded nucleic acid (A) through -L³-L⁴-, wherein -L³-L⁴- is

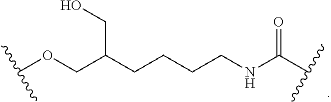 [SMILES: *OCC(CO)CCCCNC(*)=O]

In embodiments, DTx-01-43 is attached to the double-stranded nucleic acid (A) through -L³-L⁴-, wherein -L³-L⁴- is

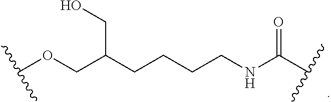 [SMILES: *OCC(CO)CCCCNC(*)=O]

In embodiments, DTx-01-43 is attached to the double-stranded nucleic acid (A) through -L³-L⁴-, wherein -L³-L⁴- is

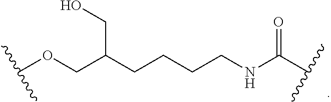 [SMILES: *OCC(CO)CCCCNC(*)=O]

In embodiments, DTx-01-44 is attached to the double-stranded nucleic acid (A) through -L³-L⁴-, wherein -L³-L⁴- is

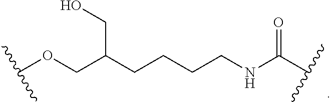 [SMILES: *OCC(CO)CCCCNC(*)=O]

In embodiments, DTx-01-45 is attached to the double-stranded nucleic acid (A) through -L³-L⁴-, wherein -L³-L⁴- is

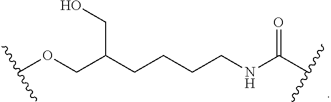 [SMILES: *OCC(CO)CCCCNC(*)=O]

In embodiments, DTx-01-46 is attached to the double-stranded nucleic acid (A) through -L³-L⁴-, wherein -L³-L⁴- is

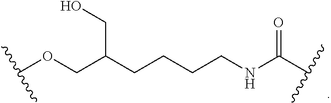 [SMILES: *OCC(CO)CCCCNC(*)=O]

In embodiments, DTx-01-50 is attached to the double-stranded nucleic acid (A) through -L³-L⁴-, wherein -L³-L⁴- is

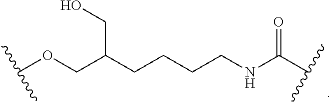 [SMILES: *OCC(CO)CCCCNC(*)=O]

In embodiments, DTx-01-51 is attached to the double-stranded nucleic acid (A) through -L³-L⁴-, wherein -L³-L⁴- is

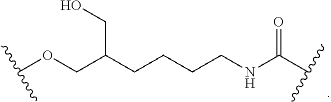 [SMILES: *OCC(CO)CCCCNC(*)=O]

In embodiments, DTx-01-52 is attached to the double-stranded nucleic acid (A) through -L³-L⁴-, wherein -L³-L⁴- is

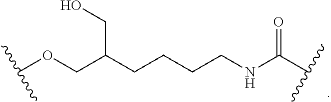 [SMILES: *OCC(CO)CCCCNC(*)=O]

In embodiments, DTx-01-53 is attached to the double-stranded nucleic acid (A) through -L³-L⁴-, wherein -L³-L⁴- is

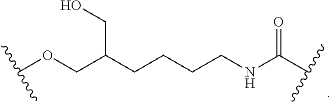 [SMILES: *OCC(CO)CCCCNC(*)=O]

In embodiments, DTx-01-54 is attached to the double-stranded nucleic acid (A) through -L³-L⁴-, wherein -L³-L⁴- is

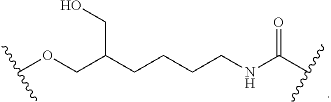 [SMILES: *OCC(CO)CCCCNC(*)=O]

In embodiments, DTx-01-55 is attached to the double-stranded nucleic acid (A) through -L³-L⁴-, wherein -L³-L⁴- is

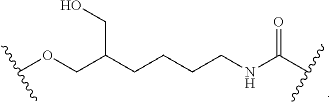 [SMILES: *OCC(CO)CCCCNC(*)=O]

In embodiments, DTx-03-06 is attached to the double-stranded nucleic acid (A) through -L³-L⁴-, wherein -L³-L⁴- is

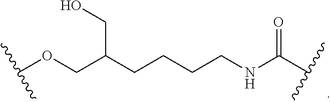 [SMILES: *OCC(CO)CCCCNC(*)=O]

In embodiments, DTx-03-50 is attached to the double-stranded nucleic acid (A) through -L³-L⁴-, wherein -L³-L⁴- is

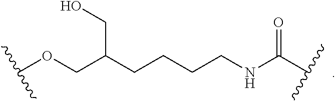 [SMILES: *OCC(CO)CCCCNC(*)=O]

In embodiments, DTx-03-51 is attached to the double-stranded nucleic acid (A) through -L³-L⁴-, wherein -L³-L⁴- is

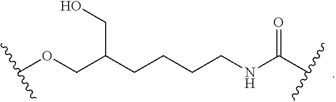 [SMILES: *OCC(CO)CCCCNC(*)=O]

In embodiments, DTx-03-52 is attached to the double-stranded nucleic acid (A) through -L³-L⁴-, wherein -L³-L⁴- is

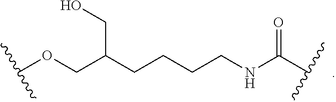 [SMILES: *OCC(CO)CCCCNC(*)=O]

In embodiments, DTx-03-53 is attached to the double-stranded nucleic acid (A) through -L³-L⁴-, wherein -L³-L⁴- is

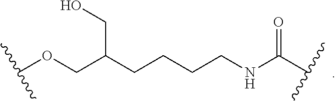 [SMILES: *OCC(CO)CCCCNC(*)=O]

In embodiments, DTx-03-54 is attached to the double-stranded nucleic acid (A) through -L³-L⁴-, wherein -L³-L⁴- is

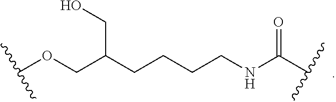 [SMILES: *OCC(CO)CCCCNC(*)=O]

In embodiments, DTx-03-55 is attached to the double-stranded nucleic acid (A) through -L³-L⁴-, wherein -L³-L⁴- is

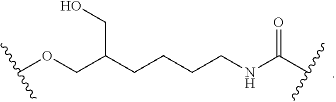 [SMILES: *OCC(CO)CCCCNC(*)=O]

In embodiments, DTx-04-01 is attached to the double-stranded nucleic acid (A) through -L³-L⁴-, wherein -L³-L⁴- is

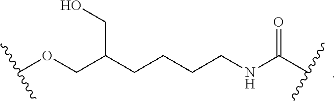 [SMILES: *OCC(CO)CCCCNC(*)=O]

In embodiments, DTx-05-01 is attached to the double-stranded nucleic acid (A) through -L³-L⁴-, wherein -L³-L⁴- is

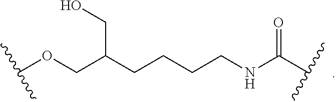 [SMILES: *OCC(CO)CCCCNC(*)=O]

In embodiments, DTx-06-06 is attached to the double-stranded nucleic acid (A) through -L³-L⁴-, wherein -L³-L⁴- is

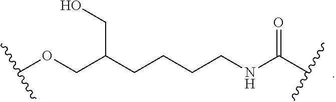 [SMILES: *OCC(CO)CCCCNC(*)=O]

In embodiments, DTx-06-50 is attached to the double-stranded nucleic acid (A) through -L³-L⁴-, wherein -L³-L⁴- is

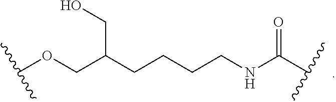 [SMILES: *OCC(CO)CCCCNC(*)=O]

In embodiments, DTx-06-51 is attached to the double-stranded nucleic acid (A) through -L³-L⁴-, wherein -L³-L⁴- is

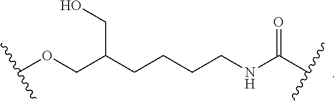 [SMILES: *OCC(CO)CCCCNC(*)=O]

In embodiments, DTx-06-52 is attached to the double-stranded nucleic acid (A) through -L³-L⁴-, wherein -L³-L⁴- is

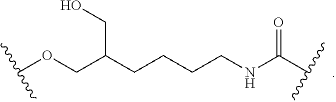 [SMILES: *OCC(CO)CCCCNC(*)=O]

In embodiments, DTx-06-53 is attached to the double-stranded nucleic acid (A) through -L³-L⁴-, wherein -L³-L⁴- is

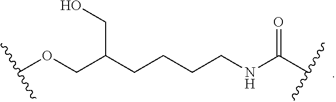 [SMILES: *OCC(CO)CCCCNC(*)=O]

In embodiments, DTx-06-54 is attached to the double-stranded nucleic acid (A) through -L³-L⁴-, wherein -L³-L⁴- is

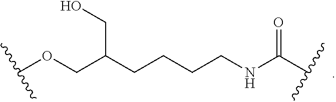 [SMILES: *OCC(CO)CCCCNC(*)=O]

In embodiments, DTx-06-55 is attached to the double-stranded nucleic acid (A) through -L³-L⁴-, wherein -L³-L⁴- is

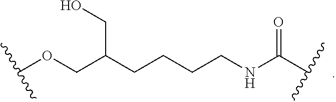 [SMILES: *OCC(CO)CCCCNC(*)=O]

In embodiments, DTx-08-01 is attached to the double-stranded nucleic acid (A) through -L³-L⁴-, wherein -L³-L⁴- is

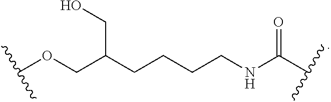 [SMILES: *OCC(CO)CCCCNC(*)=O]

In embodiments, DTx-09-01 is attached to the double-stranded nucleic acid (A) through -L³-L⁴-, wherein -L³-L⁴- is

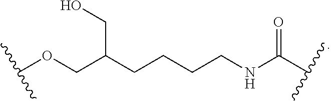 [SMILES: *OCC(CO)CCCCNC(*)=O]

In embodiments, DTx-10-01 is attached to the double-stranded nucleic acid (A) through -L³-L⁴-, wherein -L³-L⁴- is

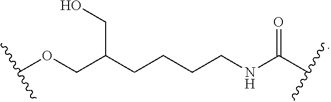 [SMILES: *OCC(CO)CCCCNC(*)=O]

In embodiments, DTx-11-01 is attached to the double-stranded nucleic acid (A) through -L³-L⁴-, wherein -L³-L⁴- is

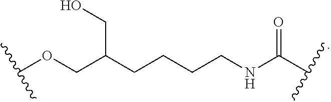 [SMILES: *OCC(CO)CCCCNC(*)=O]

In embodiments, DTx-01-60 is attached to the double-stranded nucleic acid (A) through -L³-L⁴-, wherein -L³-L⁴- is

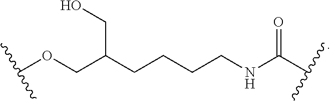 [SMILES: *OCC(CO)CCCCNC(*)=O]

In embodiments, DTx-01-61 is attached to the double-stranded nucleic acid (A) through -L³-L⁴-, wherein -L³-L⁴- is

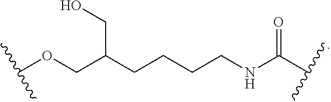 [SMILES: *OCC(CO)CCCCNC(*)=O]

In embodiments, DTx-01-62 is attached to the double-stranded nucleic acid (A) through -L³-L⁴-, wherein -L³-L⁴- is

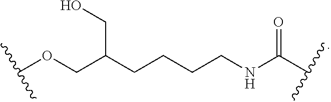 [SMILES: *OCC(CO)CCCCNC(*)=O]

In embodiments, DTx-01-63 is attached to the double-stranded nucleic acid (A) through -L³-L⁴-, wherein -L³-L⁴- is

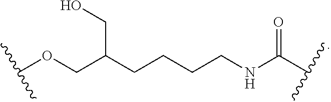 [SMILES: *OCC(CO)CCCCNC(*)=O]

In embodiments, DTx-01-64 is attached to the double-stranded nucleic acid (A) through -L³-L⁴-, wherein -L³-L⁴- is

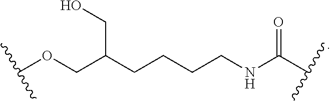 [SMILES: *OCC(CO)CCCCNC(*)=O]

In embodiments, DTx-01-65 is attached to the double-stranded nucleic acid (A) through -L³-L⁴-, wherein -L³-L⁴- is

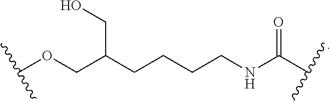 [SMILES: *OCC(CO)CCCCNC(*)=O]

In embodiments, DTx-01-66 is attached to the double-stranded nucleic acid (A) through -L³-L⁴-, wherein -L³-L⁴- is

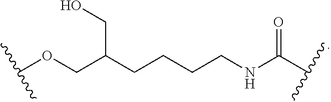 [SMILES: *OCC(CO)CCCCNC(*)=O]

In embodiments, DTx-01-67 is attached to the double-stranded nucleic acid (A) through -L³-L⁴-, wherein -L³-L⁴- is

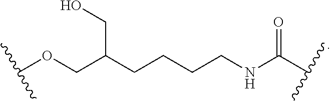 [SMILES: *OCC(CO)CCCCNC(*)=O]

In embodiments, DTx-01-68 is attached to the double-stranded nucleic acid (A) through -L³-L⁴-, wherein -L³-L⁴- is

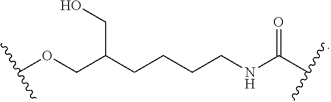 [SMILES: *OCC(CO)CCCCNC(*)=O]

In embodiments, DTx-01-69 is attached to the double-stranded nucleic acid (A) through -L³-L⁴-, wherein -L³-L⁴- is

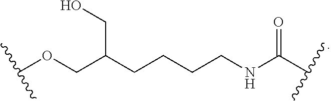 [SMILES: *OCC(CO)CCCCNC(*)=O]

In embodiments, DTx-01-70 is attached to the double-stranded nucleic acid (A) through -L³-L⁴-, wherein -L³-L⁴- is

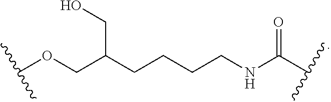 [SMILES: *OCC(CO)CCCCNC(*)=O]

In embodiments, DTx-01-71 is attached to the double-stranded nucleic acid (A) through -L³-L⁴-, wherein -L³-L⁴- is

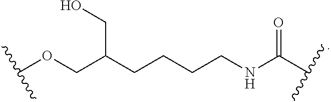 [SMILES: *OCC(CO)CCCCNC(*)=O]

In embodiments, DTx-01-72 is attached to the double-stranded nucleic acid (A) through -L³-L⁴-, wherein -L³-L⁴- is

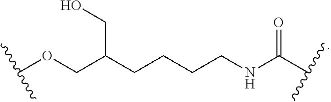 [SMILES: *OCC(CO)CCCCNC(*)=O]

In embodiments, DTx-01-73 is attached to the double-stranded nucleic acid (A) through -L³-L⁴-, wherein -L³-L⁴- is

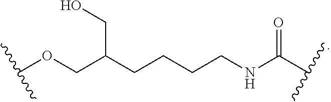 [SMILES: *OCC(CO)CCCCNC(*)=O]

In embodiments, DTx-01-74 is attached to the double-stranded nucleic acid (A) through -L³-L⁴-, wherein -L³-L⁴- is

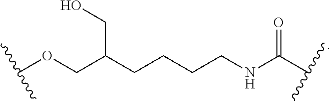 [SMILES: *OCC(CO)CCCCNC(*)=O]

In embodiments, DTx-01-75 is attached to the double-stranded nucleic acid (A) through -L³-L⁴-, wherein -L³-L⁴- is

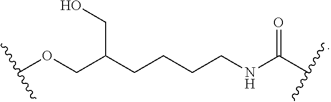 [SMILES: *OCC(CO)CCCCNC(*)=O]

In embodiments, DTx-01-76 is attached to the double-stranded nucleic acid (A) through -L³-L⁴-, wherein -L³-L⁴- is

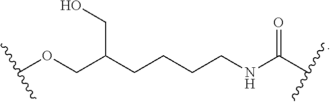 [SMILES: *OCC(CO)CCCCNC(*)=O]

In embodiments, DTx,01-77 is attached to the double-stranded nucleic acid (A) through -L³-L⁴-, wherein -L³-L⁴- is

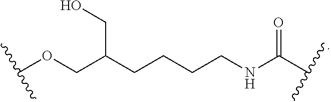 [SMILES: *OCC(CO)CCCCNC(*)=O]

In embodiments, DTx-01-78 is attached to the double-stranded nucleic acid (A) through -L³-L⁴-, wherein -L³-L⁴- is

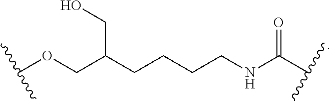 [SMILES: *OCC(CO)CCCCNC(*)=O]

In embodiments, DTx-01-79 is attached to the double-stranded nucleic acid (A) through -L³-L⁴-, wherein -L³-L⁴- is

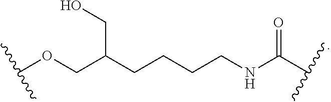 [SMILES: *OCC(CO)CCCCNC(*)=O]

In embodiments, DTx-01-80 is attached to the double-stranded nucleic acid (A) through -L³-L⁴-, wherein -L³-L⁴- is

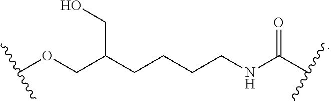 [SMILES: *OCC(CO)CCCCNC(*)=O]

In embodiments, DTx-01-81 is attached to the double-stranded nucleic acid (A) through -L³-L⁴-, wherein -L³-L⁴- is

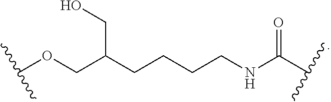 [SMILES: *OCC(CO)CCCCNC(*)=O]

In embodiments, DTx-01-82 is attached to the double-stranded nucleic acid (A) through -L³-L⁴-, wherein -L³-L⁴- is

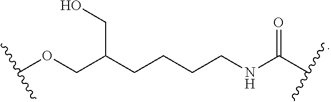 [SMILES: *OCC(CO)CCCCNC(*)=O]

In embodiments, DTx-01-83 is attached to the double-stranded nucleic acid (A) through -L³-L⁴-, wherein -L³-L⁴- is

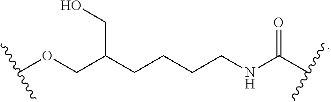 [SMILES: *OCC(CO)CCCCNC(*)=O]

In embodiments, DTx-01-84 is attached to the double-stranded nucleic acid (A) through -L³-L⁴-, wherein -L³-L⁴- is

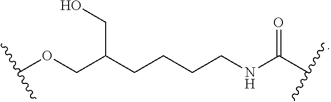 [SMILES: *OCC(CO)CCCCNC(*)=O]

In embodiments, DTx-01-85 is attached to the double-stranded nucleic acid (A) through -L³-L⁴-, wherein -L³-L⁴- is

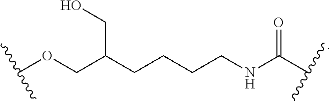 [SMILES: *OCC(CO)CCCCNC(*)=O]

In embodiments, DTx-01-86 is attached to the double-stranded nucleic acid (A) through -L³-L⁴-, wherein -L³-L⁴- is

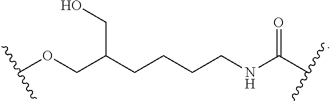 [SMILES: *OCC(CO)CCCCNC(*)=O]

In embodiments, DTx-01-87 is attached to the double-stranded nucleic acid (A) through -L³-L⁴-, wherein -L³-L⁴- is

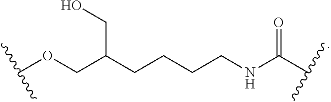 [SMILES: *OCC(CO)CCCCNC(*)=O]

In embodiments, DTx-01-88 is attached to the double-stranded nucleic acid (A) through -L³-L⁴-, wherein -L³-L⁴- is

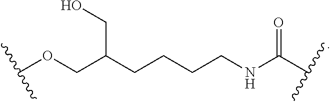 [SMILES: *OCC(CO)CCCCNC(*)=O]

In embodiments, DTx-01-89 is attached to the double-stranded nucleic acid (A) through -L³-L⁴-, wherein -L³-L⁴- is

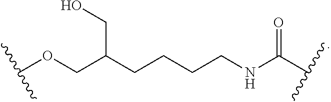 [SMILES: *OCC(CO)CCCCNC(*)=O]

In embodiments, DTx-01-90 is attached to the double-stranded nucleic acid (A) through -L³-L⁴-, wherein -L³-L⁴- is

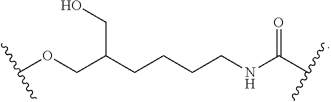 [SMILES: *OCC(CO)CCCCNC(*)=O]

In embodiments, DTx-01-91 is attached to the double-stranded nucleic acid (A) through -L³-L⁴-, wherein -L³-L⁴- is

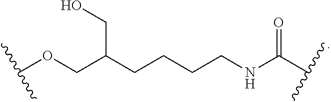 [SMILES: *OCC(CO)CCCCNC(*)=O]

In embodiments, DTx-01-92 is attached to the double-stranded nucleic acid (A) through -L³-L⁴-, wherein -L³-L⁴- is

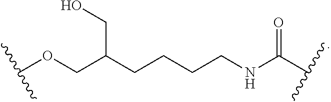 [SMILES: *OCC(CO)CCCCNC(*)=O]

In embodiments, DTx-01-93 is attached to the double-stranded nucleic acid (A) through -L³-L⁴-, wherein -L³-L⁴- is

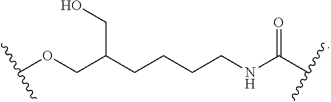 [SMILES: *OCC(CO)CCCCNC(*)=O]

In embodiments, DTx-01-94 is attached to the double-stranded nucleic acid (A) through -L³-L⁴-, wherein -L³-L⁴- is

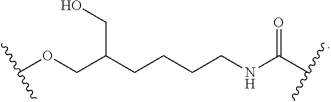 [SMILES: *OCC(CO)CCCCNC(*)=O]

In embodiments, DTx-01-95 is attached to the double-stranded nucleic acid (A) through -L³-L⁴-, wherein -L³-L⁴- is

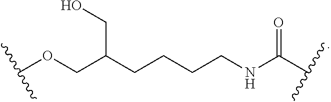 [SMILES: *OCC(CO)CCCCNC(*)=O]

In embodiments, DTx-01-96 is attached to the double-stranded nucleic acid (A) through -L³-L⁴-, wherein -L³-L⁴- is

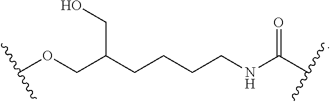 [SMILES: *OCC(CO)CCCCNC(*)=O]

In embodiments, DTx-01-97 is attached to the double-stranded nucleic acid (A) through -L³-L⁴-, wherein -L³-L⁴- is

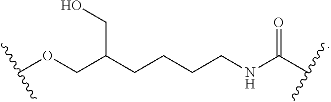 [SMILES: *OCC(CO)CCCCNC(*)=O]

in embodiments, DTx-01-98 is attached to the double-stranded nucleic acid (A) through -L³-L⁴-, wherein -L³-L⁴- is

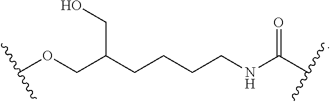 [SMILES: *OCC(CO)CCCCNC(*)=O]

In embodiments, DTx-01-99 is attached to the double-stranded nucleic acid (A) through -L³-L⁴-, wherein -L³-L⁴- is

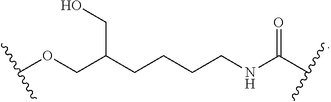 [SMILES: *OCC(CO)CCCCNC(*)=O]

In embodiments, DTx-01-100 is attached to the double-stranded nucleic acid (A) through -L³-L⁴-, wherein -L³-L⁴- is

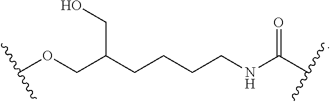 [SMILES: *OCC(CO)CCCCNC(*)=O]

In embodiments, DTx-01-101 is attached to the double-stranded nucleic acid (A) through -L³-L⁴-, wherein -L³-L⁴- is

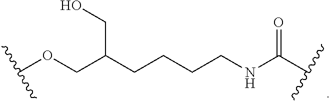 [SMILES: *OCC(CO)CCCCNC(*)=O]

In embodiments, DTx-01-01 is attached to the double-stranded nucleic acid (A) through -L³-L⁴-, wherein -L³-L⁴- is

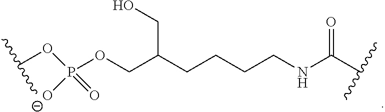 [SMILES: *OP(=O)([O-])OCC(CO)CCCCNC(*)=O]

In embodiments, DTx-01-03 is attached to the double-stranded nucleic acid (A) through -L³-L⁴-, wherein -L³-L⁴- is

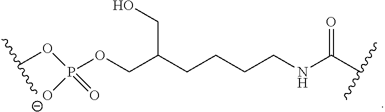 [SMILES: *OP(=O)([O-])OCC(CO)CCCCNC(*)=O]

In embodiments, DTx-01-06 is attached to the double-stranded nucleic acid (A) through -L³-L⁴-, wherein -L³-L⁴- is

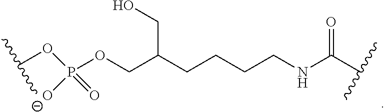 [SMILES: *OP(=O)([O-])OCC(CO)CCCCNC(*)=O]

In embodiments, DTx-01-08 is attached to the double-stranded nucleic acid (A) through -L³-L⁴-, wherein -L³-L⁴- is

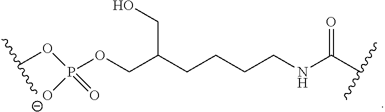 [SMILES: *OP(=O)([O-])OCC(CO)CCCCNC(*)=O]

In embodiments, DTx-01-11 is attached to the double-stranded nucleic acid (A) through -L³-L⁴-, wherein -L³-L⁴- is

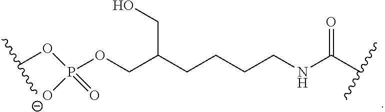 [SMILES: *OP(=O)([O-])OCC(CO)CCCCNC(*)=O]

In embodiments, DTx-01-13 is attached to the double-stranded nucleic acid (A) through -L³-L⁴-, wherein -L³-L⁴- is

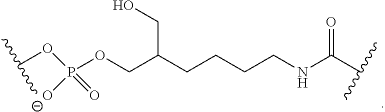 [SMILES: *OP(=O)([O-])OCC(CO)CCCCNC(*)=O]

In embodiments, DTx-01-30 is attached to the double-stranded nucleic acid (A) through -L³-L⁴-, wherein -L³-L⁴- is

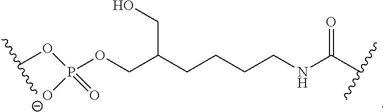 [SMILES: *OP(=O)([O-])OCC(CO)CCCCNC(*)=O]

In embodiments, DTx-01-31 is attached to the double-stranded nucleic acid (A) through -L³-L⁴-, wherein -L³-L⁴- is

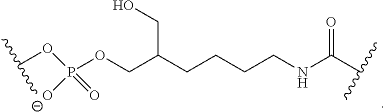 [SMILES: *OP(=O)([O-])OCC(CO)CCCCNC(*)=O]

In embodiments, DTx-01-32 is attached to the double-stranded nucleic acid (A) through -L³-L⁴-, wherein -L³-L⁴- is

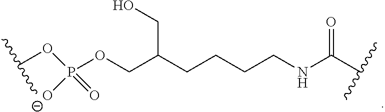 [SMILES: *OP(=O)([O-])OCC(CO)CCCCNC(*)=O]

In embodiments, DTx-01-33 is attached to the double-stranded nucleic acid (A) through -L³-L⁴-, wherein -L³-L⁴- is

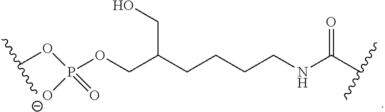 [SMILES: *OP(=O)([O-])OCC(CO)CCCCNC(*)=O]

In embodiments, DTx-01-34 is attached to the double-stranded nucleic acid (A) through -L³-L⁴-, wherein -L³-L⁴- is

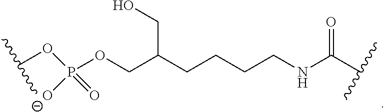 [SMILES: *OP(=O)([O-])OCC(CO)CCCCNC(*)=O]

In embodiments, DTx-01-35 is attached to the double-stranded nucleic acid (A) through -L³-L⁴-, wherein -L³-L⁴- is

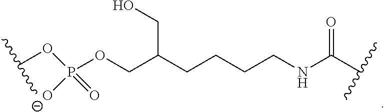 [SMILES: *OP(=O)([O-])OCC(CO)CCCCNC(*)=O]

In embodiments, DTx-01-36 is attached to the double-stranded nucleic acid (A) through -L³-L⁴-, wherein -L³-L⁴- is

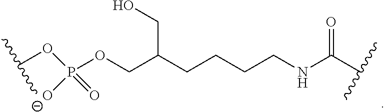 [SMILES: *OP(=O)([O-])OCC(CO)CCCCNC(*)=O]

In embodiments, DTx-01-39 is attached to the double-stranded nucleic acid (A) through -L³-L⁴-, wherein -L³-L⁴- is

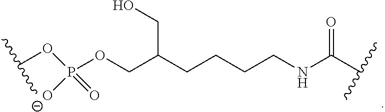 [SMILES: *OP(=O)([O-])OCC(CO)CCCCNC(*)=O]

In embodiments, DTx-01-43 is attached to the double-stranded nucleic acid (A) through -L³-L⁴-, wherein -L³-L⁴- is

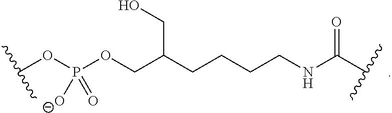 [SMILES: *OP(=O)([O-])OCC(CO)CCCCNC(*)=O]

In embodiments, DTx-01-44 is attached to the double-stranded nucleic acid (A) through -L³-L⁴-, wherein -L³-L⁴- is

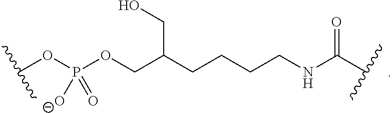 [SMILES: *OP(=O)([O-])OCC(CO)CCCCNC(*)=O]

In embodiments, DTx-01-45 is attached to the double-stranded nucleic acid (A) through -L³-L⁴-, wherein -L³-L⁴- is

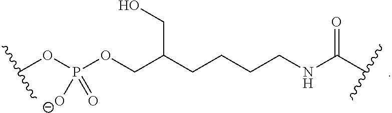 [SMILES: *OP(=O)([O-])OCC(CO)CCCCNC(*)=O]

In embodiments, DTx-01-46 is attached to the double-stranded nucleic acid (A) through -L³-L⁴-, wherein -L³-L⁴- is

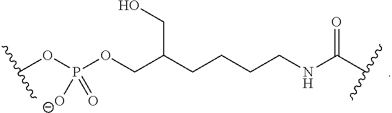 [SMILES: *OP(=O)([O-])OCC(CO)CCCCNC(*)=O]

In embodiments, DTx-01-50 is attached to the double-stranded nucleic acid (A) through -L³-L⁴-, wherein -L³-L⁴- is

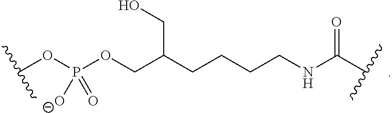 [SMILES: *OP(=O)([O-])OCC(CO)CCCCNC(*)=O]

In embodiments, DTx-01-51 is attached to the double-stranded nucleic acid (A) through -L³-L⁴-, wherein -L³-L⁴- is

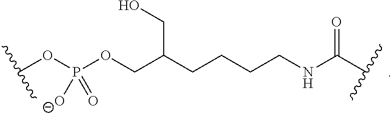 [SMILES: *OP(=O)([O-])OCC(CO)CCCCNC(*)=O]

In embodiments, DTx-01-52 is attached to the double-stranded nucleic acid (A) through -L³-L⁴-, wherein -L³-L⁴- is

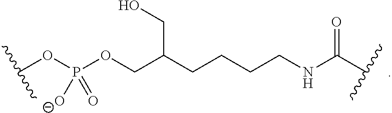 [SMILES: *OP(=O)([O-])OCC(CO)CCCCNC(*)=O]

In embodiments, DTx-01-53 is attached to the double-stranded nucleic acid (A) through -L³-L⁴-, wherein -L³-L⁴- is

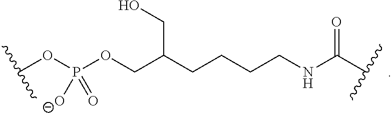 [SMILES: *OP(=O)([O-])OCC(CO)CCCCNC(*)=O]

In embodiments, DTx-01-54 is attached to the double-stranded nucleic acid (A) through -L³-L⁴-, wherein -L³-L⁴- is

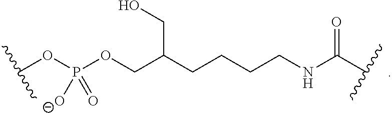 [SMILES: *OP(=O)([O-])OCC(CO)CCCCNC(*)=O]

In embodiments, DTx-01-55 is attached to the double-stranded nucleic acid (A) through -L³-L⁴-, wherein -L³-L⁴- is

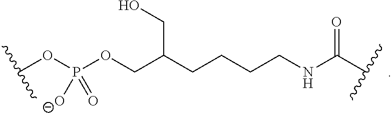 [SMILES: *OP(=O)([O-])OCC(CO)CCCCNC(*)=O]

In embodiments, DTx-03-06 is attached to the double-stranded nucleic acid (A) through -L³-L⁴-, wherein -L³-L⁴- is

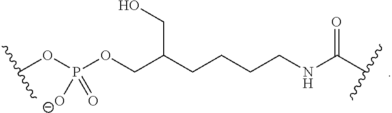 [SMILES: *OP(=O)([O-])OCC(CO)CCCCNC(*)=O]

In embodiments, DTx-03-50 is attached to the double-stranded nucleic acid (A) through -L³-L⁴-, wherein -L³-L⁴- is

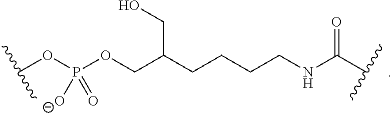 [SMILES: *OP(=O)([O-])OCC(CO)CCCCNC(*)=O]

In embodiments, DTx-03-51 is attached to the double-stranded nucleic acid (A) through -L³-L⁴-, wherein -L³-L⁴- is

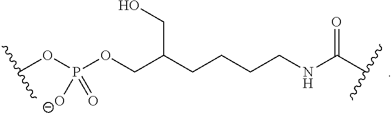 [SMILES: *OP(=O)([O-])OCC(CO)CCCCNC(*)=O]

In embodiments, DTx-03-52 is attached to the double-stranded nucleic acid (A) through -L³-L⁴-, wherein -L³-L⁴- is

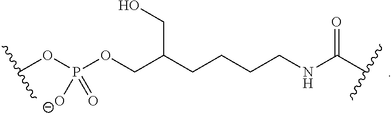 [SMILES: *OP(=O)([O-])OCC(CO)CCCCNC(*)=O]

In embodiments, DTx-03-53 is attached to the double-stranded nucleic acid (A) through -L³-L⁴-, wherein -L³-L⁴- is

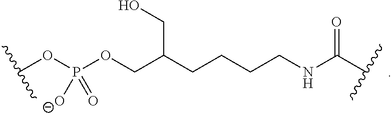 [SMILES: *OP(=O)([O-])OCC(CO)CCCCNC(*)=O]

In embodiments, DTx-03-54 is attached to the double-stranded nucleic acid (A) through -L³-L⁴-, wherein -L³-L⁴- is

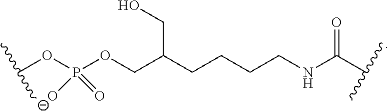 [SMILES: *OP(=O)([O-])OCC(CO)CCCCNC(*)=O]

In embodiments, DTx-03-55 is attached to the double-stranded nucleic acid (A) through -L³-L⁴-, wherein -L³-L⁴- is

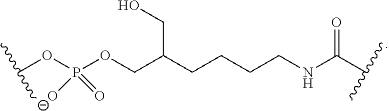 [SMILES: *OP(=O)([O-])OCC(CO)CCCCNC(*)=O]

In embodiments, DTx-04-01 is attached to the double-stranded nucleic acid (A) through -L³-L⁴-, wherein -L³-L⁴- is

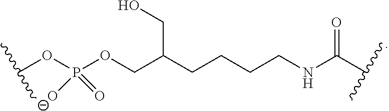 [SMILES: *OP(=O)([O-])OCC(CO)CCCCNC(*)=O]

In embodiments, DTx-05-01 is attached to the double-stranded nucleic acid (A) through -L³-L⁴-, wherein -L³-L⁴- is

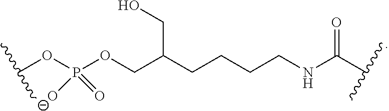 [SMILES: *OP(=O)([O-])OCC(CO)CCCCNC(*)=O]

In embodiments, DTx-06-06 is attached to the double-stranded nucleic acid (A) through -L³-L⁴-, wherein -L³-L⁴- is

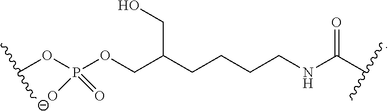 [SMILES: *OP(=O)([O-])OCC(CO)CCCCNC(*)=O]

In embodiments, DTx-06-50 is attached to the double-stranded nucleic acid (A) through -L³-L⁴-, wherein -L³-L⁴- is

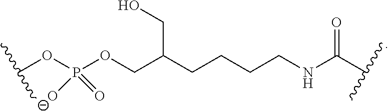 [SMILES: *OP(=O)([O-])OCC(CO)CCCCNC(*)=O]

In embodiments, DTx-06-51 is attached to the double-stranded nucleic acid (A) through -L³-L⁴-, wherein -L³-L⁴- is

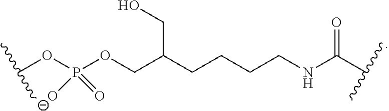 [SMILES: *OP(=O)([O-])OCC(CO)CCCCNC(*)=O]

In embodiments, DTx-06-52 is attached to the double-stranded nucleic acid (A) through -L³-L⁴-, wherein -L³-L⁴- is

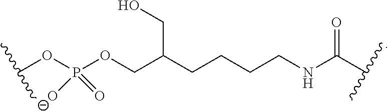 [SMILES: *OP(=O)([O-])OCC(CO)CCCCNC(*)=O]

In embodiments, DTx-06-53 is attached to the double-stranded nucleic acid (A) through -L³-L⁴-, wherein -L³-L⁴- is

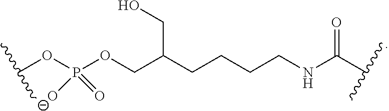 [SMILES: *OP(=O)([O-])OCC(CO)CCCCNC(*)=O]

In embodiments, DTx-06-54 is attached to the double-stranded nucleic acid (A) through -L³-L⁴-, wherein -L³-L⁴- is

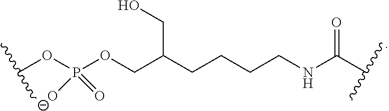 [SMILES: *OP(=O)([O-])OCC(CO)CCCCNC(*)=O]

In embodiments, DTx-06-55 is attached to the double-stranded nucleic acid (A) through -L³-L⁴-, wherein -L³-L⁴- is

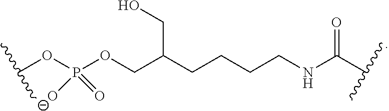 [SMILES: *OP(=O)([O-])OCC(CO)CCCCNC(*)=O]

In embodiments, DTx-08-01 is attached to the double-stranded nucleic acid (A) through -L³-L⁴-, wherein -L³-L⁴- is

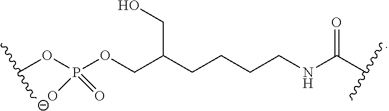 [SMILES: *OP(=O)([O-])OCC(CO)CCCCNC(*)=O]

In embodiments, DTx-09-01 is attached to the double-stranded nucleic acid (A) through -L³-L⁴-, wherein -L³-L⁴- is

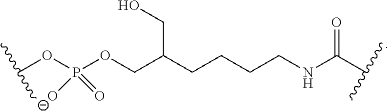 [SMILES: *OP(=O)([O-])OCC(CO)CCCCNC(*)=O]

In embodiments, DTx-10-01 is attached to the double-stranded nucleic acid (A) through -L³-L⁴-, wherein -L³-L⁴- is

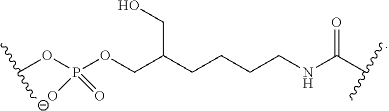 [SMILES: *OP(=O)([O-])OCC(CO)CCCCNC(*)=O]

In embodiments, DTx-11-01 is attached to the double-stranded nucleic acid (A) through -L³-L⁴-, wherein -L³-L⁴- is

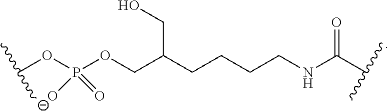 [SMILES: *OP(=O)([O-])OCC(CO)CCCCNC(*)=O]

In embodiments, DTx-01-60 is attached to the double-stranded nucleic acid (A) through -L³-L⁴-, wherein -L³-L⁴- is

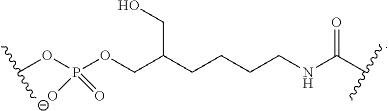 [SMILES: *OP(=O)([O-])OCC(CO)CCCCNC(*)=O]

In embodiments, DTx-01-61 is attached to the double-stranded nucleic acid (A) through -L³-L⁴-, wherein -L³-L⁴- is

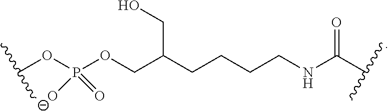 [SMILES: *OP(=O)([O-])OCC(CO)CCCCNC(*)=O]

In embodiments, DTx-01-62 is attached to the double-stranded nucleic acid (A) through -L³-L⁴-, wherein -L³-L⁴- is

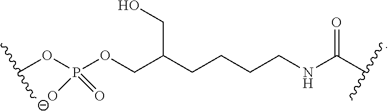 [SMILES: *OP(=O)([O-])OCC(CO)CCCCNC(*)=O]

In embodiments, DTx-01-63 is attached to the double-stranded nucleic acid (A) through -L³-L⁴-, wherein -L³-L⁴- is

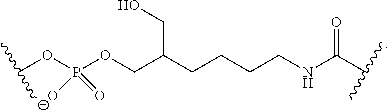 [SMILES: *OP(=O)([O-])OCC(CO)CCCCNC(*)=O]

In embodiments, DTx-01-64 is attached to the double-stranded nucleic acid (A) through -L³-L⁴-, wherein -L³-L⁴- is

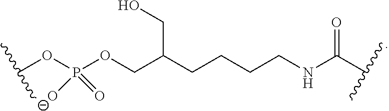 [SMILES: *OP(=O)([O-])OCC(CO)CCCCNC(*)=O]

In embodiments, DTx-01-65 is attached to the double-stranded nucleic acid (A) through -L³-L⁴-, wherein -L³-L⁴- is

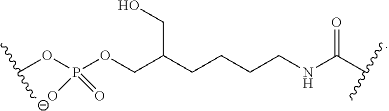 [SMILES: *OP(=O)([O-])OCC(CO)CCCCNC(*)=O]

In embodiments, DTx-01-66 is attached to the double-stranded nucleic acid (A) through -L³-L⁴-, wherein -L³-L⁴- is

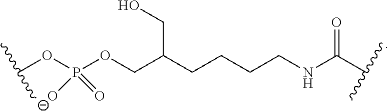 [SMILES: *OP(=O)([O-])OCC(CO)CCCCNC(*)=O]

In embodiments, DTx-01-67 is attached to the double-stranded nucleic acid (A) through -L³-L⁴-, wherein -L³-L⁴- is

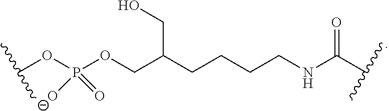 [SMILES: *OP(=O)([O-])OCC(CO)CCCCNC(*)=O]

In embodiments, DTx-01-68 is attached to the double-stranded nucleic acid (A) through -L³-L⁴-, wherein -L³-L⁴- is

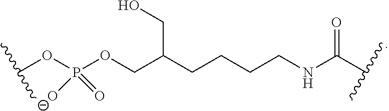 [SMILES: *OP(=O)([O-])OCC(CO)CCCCNC(*)=O]

In embodiments, DTx-01-69 is attached to the double-stranded nucleic acid (A) through -L³-L⁴-, wherein -L³-L⁴- is

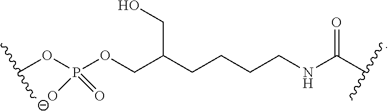 [SMILES: *OP(=O)([O-])OCC(CO)CCCCNC(*)=O]

In embodiments, DTx-01-70 is attached to the double-stranded nucleic acid (A) through -L³-L⁴-, wherein -L³-L⁴- is

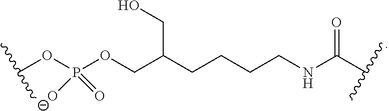 [SMILES: *OP(=O)([O-])OCC(CO)CCCCNC(*)=O]

In embodiments, DTx-01-71 is attached to the double-stranded nucleic acid (A) through -L³-L⁴-, wherein -L³-L⁴- is

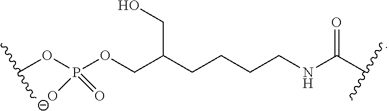 [SMILES: *OP(=O)([O-])OCC(CO)CCCCNC(*)=O]

In embodiments, DTx-01-72 is attached to the double-stranded nucleic acid (A) through -L³-L⁴-, wherein -L³-L⁴- is

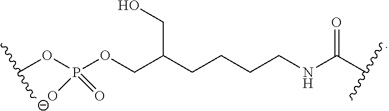 [SMILES: *OP(=O)([O-])OCC(CO)CCCCNC(*)=O]

In embodiments, DTx-01-73 is attached to the double-stranded nucleic acid (A) through -L³-L⁴-, wherein -L³-L⁴- is

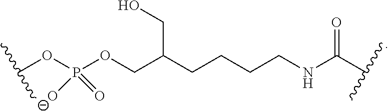 [SMILES: *OP(=O)([O-])OCC(CO)CCCCNC(*)=O]

In embodiments, DTx-01-74 is attached to the double-stranded nucleic acid (A) through -L³-L⁴-, wherein -L³-L⁴- is

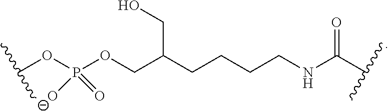 [SMILES: *OP(=O)([O-])OCC(CO)CCCCNC(*)=O]

In embodiments, DTx-01-75 is attached to the double-stranded nucleic acid (A) through -L³-L⁴-, wherein -L³-L⁴- is

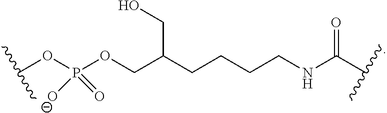 [SMILES: *OP(=O)([O-])OCC(CO)CCCCNC(*)=O]

In embodiments, DTx-01-76 is attached to the double-stranded nucleic acid (A) through -L³-L⁴-, wherein -L³-L⁴- is

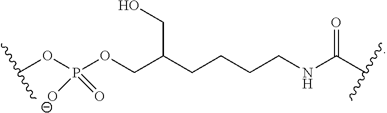 [SMILES: *OP(=O)([O-])OCC(CO)CCCCNC(*)=O]

In embodiments, DTx-01-77 is attached to the double-stranded nucleic acid (A) through -L³-L⁴-, wherein -L³-L⁴- is

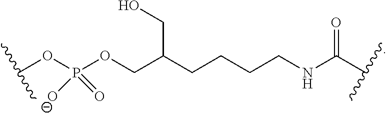 [SMILES: *OP(=O)([O-])OCC(CO)CCCCNC(*)=O]

In embodiments, DTx-01-78 is attached to the double-stranded nucleic acid (A) through -L³-L⁴-, wherein -L³-L⁴- is

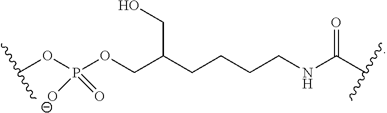 [SMILES: *OP(=O)([O-])OCC(CO)CCCCNC(*)=O]

In embodiments, DTx-01-79 is attached to the double-stranded nucleic acid (A) through -L³-L⁴-, wherein -L³-L⁴- is

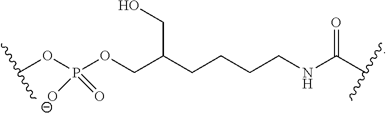 [SMILES: *OP(=O)([O-])OCC(CO)CCCCNC(*)=O]

In embodiments, DTx-01-80 is attached to the double-stranded nucleic acid (A) through -L³-L⁴-, wherein -L³-L⁴- is

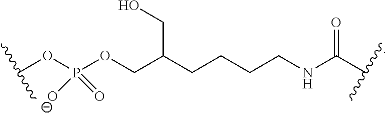 [SMILES: *OP(=O)([O-])OCC(CO)CCCCNC(*)=O]

In embodiments, DTx-01-81 is attached to the double-stranded nucleic acid (A) through -L³-L⁴-, wherein -L³-L⁴- is

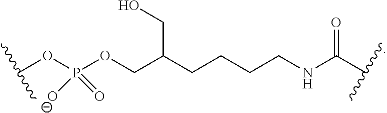 [SMILES: *OP(=O)([O-])OCC(CO)CCCCNC(*)=O]

In embodiments, DTx-01-82 is attached to the double-stranded nucleic acid (A) through -L³-L⁴-, wherein -L³-L⁴- is

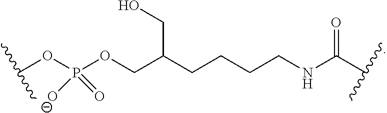 [SMILES: *OP(=O)([O-])OCC(CO)CCCCNC(*)=O]

In embodiments, DTx-01-83 is attached to the double-stranded nucleic acid (A) through -L³-L⁴-, wherein -L³-L⁴- is

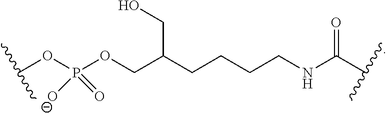 [SMILES: *OP(=O)([O-])OCC(CO)CCCCNC(*)=O]

In embodiments, DTx-01-84 is attached to the double-stranded nucleic acid (A) through -L³-L⁴-, wherein -L³-L⁴- is

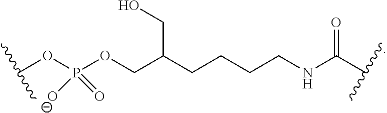 [SMILES: *OP(=O)([O-])OCC(CO)CCCCNC(*)=O]

In embodiments, DTx-01-85 is attached to the double-stranded nucleic acid (A) through -L³-L⁴-, wherein -L³-L⁴- is

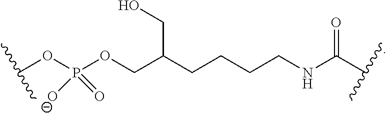 [SMILES: *OP(=O)([O-])OCC(CO)CCCCNC(*)=O]

In embodiments, DTx-01-86 is attached to the double-stranded nucleic acid (A) through -L³-L⁴-, wherein -L³-L⁴- is

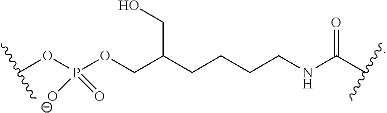 [SMILES: *OP(=O)([O-])OCC(CO)CCCCNC(*)=O]

In embodiments, DTx-01-87 is attached to the double-stranded nucleic acid (A) through -L³-L⁴-, wherein -L³-L⁴- is

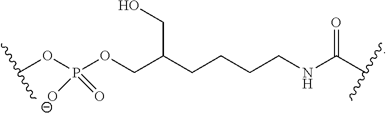 [SMILES: *OP(=O)([O-])OCC(CO)CCCCNC(*)=O]

In embodiments, DTx-01-88 is attached to the double-stranded nucleic acid (A) through -L³-L⁴-, wherein -L³-L⁴- is

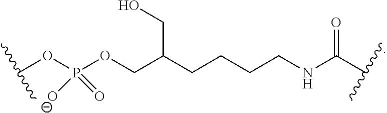 [SMILES: *OP(=O)([O-])OCC(CO)CCCCNC(*)=O]

In embodiments, DTx-01-89 is attached to the double-stranded nucleic acid (A) through -L³-L⁴-, wherein -L³-L⁴- is

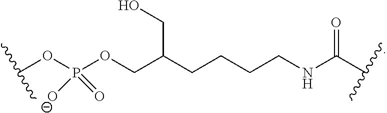 [SMILES: *OP(=O)([O-])OCC(CO)CCCCNC(*)=O]

In embodiments, DTx-01-90 is attached to the double-stranded nucleic acid (A) through -L³-L⁴-, wherein -L³-L⁴- is

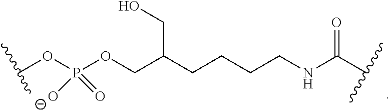 [SMILES: *OP(=O)([O-])OCC(CO)CCCCNC(*)=O]

In embodiments, DTx-01-91 is attached to the double-stranded nucleic acid (A) through -L³-L⁴-, wherein -L³-L⁴- is

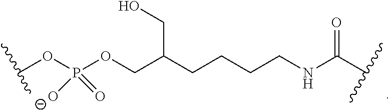 [SMILES: *OP(=O)([O-])OCC(CO)CCCCNC(*)=O]

In embodiments, DTx-01-92 is attached to the double-stranded nucleic acid (A) through -L³-L⁴-, wherein -L³-L⁴- is

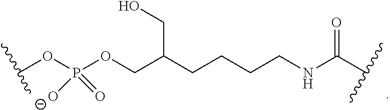 [SMILES: *OP(=O)([O-])OCC(CO)CCCCNC(*)=O]

In embodiments, DTx-01-93 is attached to the double-stranded nucleic acid (A) through -L³-L⁴-, wherein -L³-L⁴- is

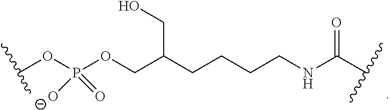 [SMILES: *OP(=O)([O-])OCC(CO)CCCCNC(*)=O]

In embodiments, DTx-01-94 is attached to the double-stranded nucleic acid (A) through -L³-L⁴-, wherein -L³-L⁴- is

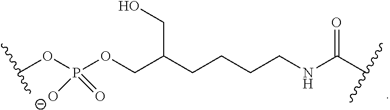 [SMILES: *OP(=O)([O-])OCC(CO)CCCCNC(*)=O]

In embodiments, DTx-01-95 is attached to the double-stranded nucleic acid (A) through -L³-L⁴-, wherein -L³-L⁴- is

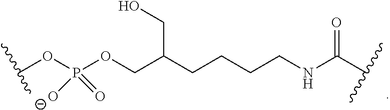 [SMILES: *OP(=O)([O-])OCC(CO)CCCCNC(*)=O]

In embodiments, DTx-01-96 is attached to the double-stranded nucleic acid (A) through -L³-L⁴-, wherein -L³-L⁴- is

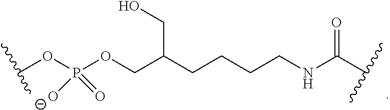 [SMILES: *OP(=O)([O-])OCC(CO)CCCCNC(*)=O]

In embodiments, DTx-01-97 is attached to the double-stranded nucleic acid (A) through -L³-L⁴-, wherein -L³-L⁴- is

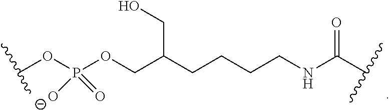 [SMILES: *OP(=O)([O-])OCC(CO)CCCCNC(*)=O]

In embodiments, DTx-01-98 is attached to the double-stranded nucleic acid (A) through -L³-L⁴-, wherein -L³-L⁴- is

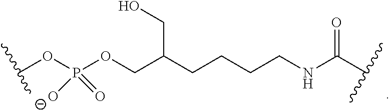 [SMILES: *OP(=O)([O-])OCC(CO)CCCCNC(*)=O]

In embodiments, DTx-01-99 is attached to the double-stranded nucleic acid (A) through -L³-L⁴-, wherein -L³-L⁴- is

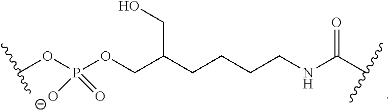 [SMILES: *OP(=O)([O-])OCC(CO)CCCCNC(*)=O]

In embodiments, DTx-01-100 is attached to the double-stranded nucleic acid (A) through -L³-L⁴-, wherein -L³-L⁴- is

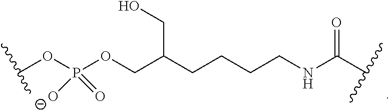 [SMILES: *OP(=O)([O-])OCC(CO)CCCCNC(*)=O]

In embodiments, DTx-01-101 is attached to the double-stranded nucleic acid (A) through -L³-L⁴-, wherein -L³-L⁴- is

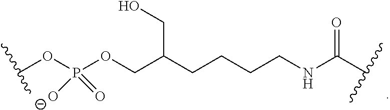 [SMILES: *OP(=O)([O-])OCC(CO)CCCCNC(*)=O]

In embodiments, -L³-L⁴- is

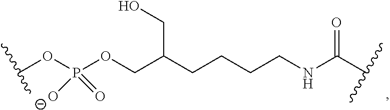 [SMILES: *OP(=O)([O-])OCC(CO)CCCCNC(*)=O]

the phosphate group is attached to the 3′ carbon of the 3′ terminal nucleotide of the sense strand, L⁶ is

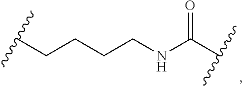 [SMILES: *CCCCNC(*)=O]

L⁵ is —NHC(O)—, R³ is hydrogen, R¹ is unsubstituted unbranched C₁₅ alkyl, and R² is unsubstituted unbranched C₁₅ alkyl.

In embodiments, -L³-L⁴- is

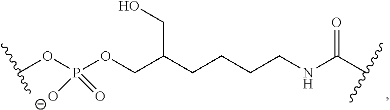 [SMILES: *OP(=O)([O-])OCC(CO)CCCCNC(*)=O]

the phosphate group of -L³-L⁴- is attached to the 3′ carbon of the 3′ terminal nucleotide of the sense strand, L⁶ is

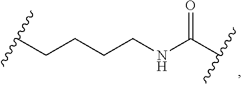 [SMILES: *CCCCNC(*)=O]

L⁵ is —NHC(O)—, R³ is hydrogen, R¹ is unsubstituted unbranched C₁₃ alkyl, and R² is unsubstituted unbranched C₁₃ alkyl.

In embodiments, -L³-L⁴- is

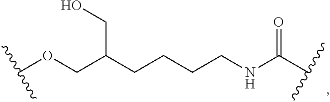 [SMILES: *OCC(CO)CCCCNC(*)=O]

within -L³-L⁴-, -L³ is attached to a phosphate group at the 3′ carbon of the 3′ terminal nucleotide of the sense strand, L⁶ is

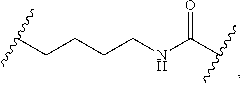 [SMILES: *CCCCNC(*)=O]

L⁵ is —NHC(O)—, R³ is hydrogen, R¹ is unsubstituted unbranched C₁₅ alkyl, and R² is unsubstituted unbranched C₁₅ alkyl.

In embodiments, -L³-L⁴- is

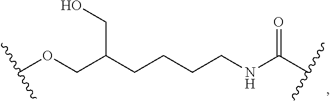 [SMILES: *OCC(CO)CCCCNC(*)=O]

within -L³-L⁴-, -L³ is attached to a phosphate group at the 3′ carbon of the 3′ terminal nucleotide of the sense strand, L⁶ is

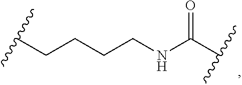 [SMILES: *CCCCNC(*)=O]

L⁵ is —NHC(O)—, R³ is hydrogen, R¹ is unsubstituted unbranched C₁₃ alkyl, and R² is unsubstituted unbranched C₁₃ alkyl.

In embodiments, a compound is DT-000623, where -L³-L⁴- is

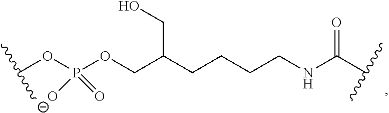 [SMILES: *OP(=O)([O-])OCC(CO)CCCCNC(*)=O]

the phosphate group of -L³-L⁴- is attached to the 3′ carbon of the 3′ terminal nucleotide of the sense strand, L⁶ is

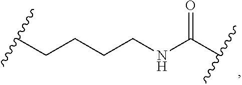 [SMILES: *CCCCNC(*)=O]

L⁵ is —NHC(O)—, R³ is hydrogen, R¹ is unsubstituted unbranched C₁₅ alkyl, R² is unsubstituted unbranched C₁₅ alkyl, the nucleotide sequence of the sense strand is
5′-OH—U_{F}^{S}C_{M}^{S}C_{F}U_{M}G_{F}U_{M}U_{F}G_{M}C_{F}U_{M}G_{F}A_{M}G_{F}U_{M}A_{F}U_{M}C_{F}^{S}A_{M}^{S}U_{F}-3′ (SEQ ID NO: 652), and the nucleotide sequence of the antisense strand is
5′-PO4-A_{M}^{S}U_{F}^{S}G_{M}A_{F}U_{M}A_{F}C_{M}U_{F}C_{M}A_{F}G_{M}C_{F}A_{M}A_{F}C_{M}A_{F}G_{M}G_{F}A_{M}^{S}T_{D}^{S}T_{D}-OH-3′ (SEQ ID NO: 176), where a nucleotide followed by the subscript “F” is a 2′-fluoro nucleotide; a nucleotide followed by the subscript “M” is a 2′-O-methyl nucleotide; a nucleotide followed by a subscript “D” is a beta-D-deoxyribonucleotide; a superscript “S” is a phosphorothioate internucleotide linkage; and all other internucleotide linkages are phosphodiester internucleotide linkages. “5′-OH” and “OH-3′” are hydroxyl moieties at the 5′-terminus and 3′ terminus, respectively.

In embodiments, a compound is DT-000812, where -L³-L⁴- is

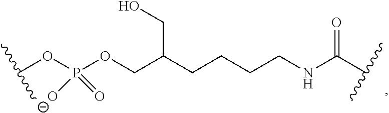 [SMILES: *OP(=O)([O-])OCC(CO)CCCCNC(*)=O]

the phosphate group of -L³-L⁴- is attached to the 3′ carbon of the 3′ terminal nucleotide of the sense strand, L⁶ is

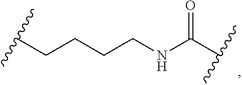 [SMILES: *CCCCNC(*)=O]

L⁵ is —NHC(O)—, R³ is hydrogen, R¹ is unsubstituted unbranched C₁₅ alkyl, R² is unsubstituted unbranched C₁₅ alkyl, the nucleotide sequence of the sense strand is
5′-OH—C_{F}^{S}C_{M}^{S}U_{F}C_{M}C_{F}U_{M}G_{F}U_{M}U_{F}G_{M}C_{F}U_{M}G_{F}A_{M}G_{F}U_{M}A_{F}U_{M}C_{F}^{S}A_{M}^{S}U_{F}-3′ (SEQ ID NO: 658), and the nucleotide sequence of the antisense strand is
5′-VP-A_{M}^{S}U_{F}^{S}G_{M}A_{F}U_{M}A_{F}C_{M}U_{F}C_{M}A_{F}G_{M}C_{F}A_{M}A_{F}C_{M}A_{F}G_{M}G_{F}A_{M}G_{F}G_{M}^{S}A_{M}^{S}GM-OH-3′ (SEQ ID NO: 879), where a nucleotide followed by the subscript “F” is a 2′-fluoro nucleotide; a nucleotide followed by the subscript “M” is a 2′-O-methyl nucleotide; a superscript “S” is a phosphorothioate internucleotide linkage; and all other internucleotide linkages are phosphodiester internucleotide linkages. “5′-VP” is a 5′-vinylphosphonate at the 5′-terminal nucleotide of the antisense strand. “5′-OH” and “OH-3′” are hydroxyl moieties at the 5′-terminus and 3′ terminus, respectively.

In embodiments, a compound is DT-001246, where -L³-L⁴- is

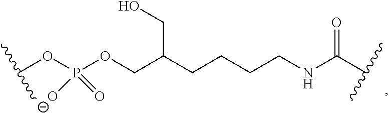 [SMILES: *OP(=O)([O-])OCC(CO)CCCCNC(*)=O]

the phosphate group of -L³-L⁴- is attached to the 3′ carbon of the 3′ terminal nucleotide of the sense strand, L⁶ is

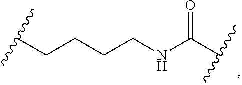 [SMILES: *CCCCNC(*)=O]

L⁵ is —NHC(O)—, R³ is hydrogen, R¹ is unsubstituted unbranched C₁₅ alkyl, R² is unsubstituted unbranched C₁₅ alkyl, the nucleotide sequence of the sense strand is
5′-OH—C_{F}^{S}C_{M}^{S}U_{F}C_{M}C_{F}U_{M}G_{F}U_{M}U_{F}G_{M}C_{F}U_{F}G_{F}A_{M}G_{F}U_{M}A_{F}U_{M}C_{F}^{S}A_{M}^{S}U_{F}-3′ (SEQ ID NO: 770), and the nucleotide sequence of the antisense strand is
5′-VP-A_{M}^{S}U_{F}^{S}G_{M}A_{F}U_{M}A_{F}C_{M}U_{F}C_{M}A_{M}G_{M}C_{F}A_{M}A_{F}C_{M}A_{F}G_{M}G_{F}A_{M}G_{F}G_{M}^{S}A_{M}^{S}GM-OH-3′ (SEQ ID NO: 899), where a nucleotide followed by the subscript “F” is a 2′-fluoro nucleotide; a nucleotide followed by the subscript “M” is a 2′-O-methyl nucleotide; a superscript “S” is a phosphorothioate internucleotide linkage; and all other internucleotide linkages are phosphodiester internucleotide linkages. “5′-VP” is a 5′-vinylphosphonate at the 5′-terminal nucleotide of the antisense strand. “5′-OH” and “OH-3′” are hydroxyl moieties at the 5′-terminus and 3′ terminus, respectively.

In embodiments, a compound is DT-001247, where -L³-L⁴- is

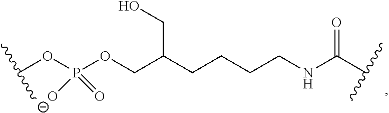 [SMILES: *OP(=O)([O-])OCC(CO)CCCCNC(*)=O]

the phosphate group of -L³-L⁴- is attached to the 3′ carbon of the 3′ terminal nucleotide of the sense strand, L⁶ is

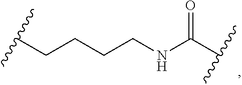 [SMILES: *CCCCNC(*)=O]

L⁵ is —NHC(O)—, R³ is hydrogen, R¹ is unsubstituted unbranched C₁₅ alkyl, R² is unsubstituted unbranched C₁₅ alkyl, the nucleotide sequence of the sense strand is
5′-OH—C_{F}^{S}C_{M}^{S}U_{F}C_{M}C_{F}U_{M}G_{F}U_{M}U_{F}G_{F}C_{F}U_{M}G_{F}A_{M}G_{F}U_{M}A_{F}U_{M}C_{F}^{S}A_{M}^{S}U_{F}-3′ (SEQ ID NO: 771), and the nucleotide sequence of the antisense strand is
5′-VP-A_{M}^{S}U_{F}^{S}G_{M}A_{F}U_{M}A_{F}C_{M}U_{F}C_{M}A_{F}G_{M}C_{M}A_{M}A_{F}C_{M}A_{F}G_{M}G_{F}A_{M}G_{F}G_{M}^{S}A_{M}^{S}GM-OH-3′ (SEQ ID NO: 900), where a nucleotide followed by the subscript “F” is a 2′-fluoro nucleotide; a nucleotide followed by the subscript “M” is a 2′-O-methyl nucleotide; a superscript “S” is a phosphorothioate internucleotide linkage; and all other internucleotide linkages are phosphodiester internucleotide linkages. “5′-VP” is a 5′-vinyl phosphonate at the 5′-terminal nucleotide of the antisense strand. “5′-OH” and “OH-3′” are hydroxyl moieties at the 5′-terminus and 3′ terminus, respectively.

In embodiments, a compound is DT-001250, where -L³-L⁴- is

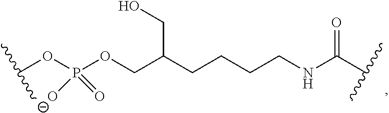 [SMILES: *OP(=O)([O-])OCC(CO)CCCCNC(*)=O]

the phosphate group of -L³-L⁴- is attached to the 3′ carbon of the 3′ terminal nucleotide of the sense strand, L⁶ is

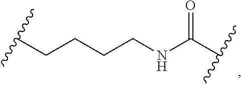 [SMILES: *CCCCNC(*)=O]

L⁵ is —NHC(O)—R³ is hydrogen, R¹ is unsubstituted unbranched C₁₅ alkyl, R² is unsubstituted unbranched C₁₅ alkyl, the nucleotide sequence of the sense strand is
5′-OH-C_{M}^{S}C_{M}^{S}U_{M}C_{M}C_{M}U_{M}G_{F}U_{M}U_{F}G_{M}C_{F}U_{M}G_{F}A_{M}G_{F}U_{M}A_{F}U_{M}C_{M}^{S}A_{M}^{S}UM-3′ (SEQ ID NO: 772), and the nucleotide sequence of the antisense strand is
5′-VP-A_{M}^{S}U_{F}^{S}G_{M}A_{F}U_{M}A_{F}C_{M}U_{F}C_{M}A_{M}G_{M}C_{F}A_{M}A_{F}C_{M}A_{F}G_{M}G_{F}A_{M}G_{F}G_{M}^{S}A_{M}^{S}GM-OH-3′ (SEQ ID NO: 899), where a nucleotide followed by the subscript “F” is a 2′-fluoro nucleotide; a nucleotide followed by the subscript “M” is a 2′-O-methyl nucleotide; a superscript “S” is a phosphorothioate internucleotide linkage; and all other internucleotide linkages are phosphodiester internucleotide linkages. “5′-VP” is a 5′-vinylphosphonate at the 5′-terminal nucleotide of the antisense strand. “5′-OH” and “OH-3′” are hydroxyl moieties at the 5′-terminus and 3′ terminus, respectively.

In embodiments, a compound is DT-001247, where -L³-L⁴- is

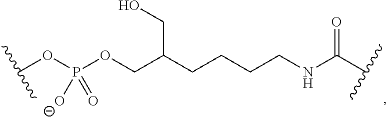 [SMILES: *OP(=O)([O-])OCC(CO)CCCCNC(*)=O]

the phosphate group of -L³-L⁴- is attached to the 3′ carbon of the 3′ terminal nucleotide of the sense strand, L⁶ is

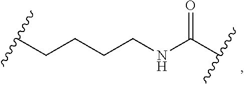 [SMILES: *CCCCNC(*)=O]

L⁵ is —NHC(O)—, R³ is hydrogen, R¹ is unsubstituted unbranched C₁₅ alkyl, R² is unsubstituted unbranched C₁₅ alkyl, the nucleotide sequence of the sense strand is
5′-OH—C_{F}^{S}C_{M}^{S}U_{F}C_{M}C_{F}U_{M}G_{F}U_{M}U_{F}G_{F}C_{F}U_{M}G_{F}A_{M}G_{F}U_{M}A_{F}U_{M}C_{F}^{S}A_{M}^{S}U_{F}-3′ (SEQ ID ID NO: 773), and the nucleotide sequence of the antisense strand is
5′-VP-A_{M}^{S}U_{F}^{S}G_{M}A_{F}U_{M}A_{F}C_{M}U_{M}C_{M}A_{F}G_{M}C_{M}A_{M}A_{F}C_{M}A_{F}G_{M}G_{F}A_{M}G_{F}G_{M}^{S}A_{M}^{S}GM-OH-3′ (SEQ ID NO: 901), where a nucleotide followed by the subscript “F” is a 2′-fluoro nucleotide; a nucleotide followed by the subscript “M” is a 2′-O-methyl nucleotide; a superscript “S” is a phosphorothioate internucleotide linkage; and all other internucleotide linkages are phosphodiester internucleotide linkages. “5′-VP” is a 5′-VP modification at the 5′-terminal nucleotide of the antisense strand. “5′-OH” and “OH-3′” are hydroxyl moieties at the 5′-terminus and 3′ terminus, respectively.

In embodiments, a compound is DT-001252, where -L³-L⁴- is

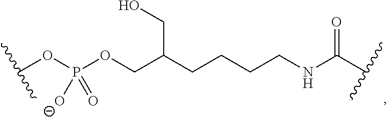 [SMILES: *OP(=O)([O-])OCC(CO)CCCCNC(*)=O]

the phosphate group of -L³-L⁴- is attached to the 3′ carbon of the 3′ terminal nucleotide of the sense strand, L⁶ is

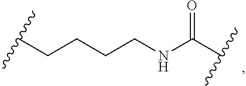 [SMILES: *CCCCNC(*)=O]

L⁵ is —NHC(O)—, R³ is hydrogen, R¹ is unsubstituted unbranched C₁₅ alkyl, R² is unsubstituted unbranched C₁₅ alkyl, the nucleotide sequence of the sense strand is
5′-OH-C_{M}^{S}C_{M}^{S}U_{M}C_{M}C_{M}U_{M}G_{F}U_{M}U_{F}G_{F}C_{F}U_{M}G_{M}A_{M}G_{M}U_{M}A_{M}U_{M}C_{M}^{S}A_{M}^{S}UM-3′ (SEQ ID NO: 774), and the nucleotide sequence of the antisense strand is
5′-VP-A_{M}^{S}UF^{S}G_{M}A_{M}U_{M}A_{F}C_{M}U_{M}C_{M}A_{M}G_{M}C_{M}A_{M}A_{F}C_{M}A_{F}G_{M}G_{M}A_{M}G_{M}G_{M}^{S}A_{M}^{S}G_{M}-OH-3′ (SEQ ID NO: 902), where a nucleotide followed by the subscript “F” is a 2′-fluoro nucleotide; a nucleotide followed by the subscript “M” is a 2′-O-methyl nucleotide; a superscript “S” is a phosphorothioate internucleotide linkage; and all other internucleotide linkages are phosphodiester internucleotide linkages. “5′-VP” is a 5′-VP modification at the
5′-terminal nucleotide of the antisense strand. “5′-OH” and “OH-3′” are hydroxyl moieties at the 5′-terminus and 3′ terminus, respectively.

In embodiments, a compound is DT-001253, where -L³-L⁴- is

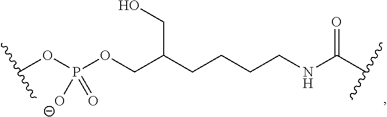 [SMILES: *OP(=O)([O-])OCC(CO)CCCCNC(*)=O]

the phosphate group of -L³-L⁴- is attached to the 3′ carbon of the 3′ terminal nucleotide of the sense strand, L⁶ is

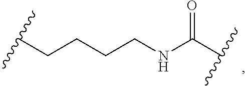 [SMILES: *CCCCNC(*)=O]

L⁵ is —NHC(O)—, R³ is hydrogen, R¹ is unsubstituted unbranched C₁₅ alkyl, R² is unsubstituted unbranched C₁₅ alkyl, the nucleotide sequence of the sense strand is
5′-OH-C_{M}^{S}C_{M}^{S}U_{M}C_{M}C_{M}U_{M}G_{F}U_{M}U_{F}G_{F}C_{F}U_{M}G_{M}A_{M}G_{M}U_{M}A_{M}U_{M}C_{M}AMUM-3′ (SEQ ID NO: 775), and the nucleotide sequence of the antisense strand is
5′-VP-A_{M}^{S}U_{F}^{S}G_{M}A_{M}U_{M}A_{F}C_{M}U_{M}C_{M}A_{M}G_{M}C_{M}A_{M}A_{F}C_{M}A_{F}G_{M}G_{M}A_{M}G_{M}G_{M}^{S}A_{M}^{S}G_{M}-OH-3′ (SEQ ID NO: 902), where a nucleotide followed by the subscript “F” is a 2′-fluoro nucleotide; a nucleotide followed by the subscript “M” is a 2′-O-methyl nucleotide; a superscript “S” is a phosphorothioate internucleotide linkage; and all other internucleotide linkages are phosphodiester internucleotide linkages. “5′-VP” is a 5′-VP modification at the 5′-terminal nucleotide of the antisense strand. “5′-OH” and “OH-3′” are hydroxyl moieties at the 5′-terminus and 3′ terminus, respectively.

In embodiments, a compound is DT-001254, where -L³-L⁴- is

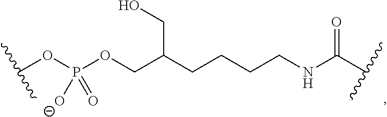 [SMILES: *OP(=O)([O-])OCC(CO)CCCCNC(*)=O]

the phosphate group of -L³-L⁴- is attached to the 3′ carbon of the 3′ terminal nucleotide of the sense strand, L⁶ is

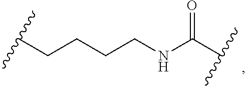 [SMILES: *CCCCNC(*)=O]

L⁵ is —NHC(O)—, R³ is hydrogen, R¹ is unsubstituted unbranched C₁₅ alkyl, R² is unsubstituted unbranched C₁₅ alkyl, the nucleotide sequence of the sense strand is
5′-OH-C_{E}^{S}C_{E}^{S}U_{M}C_{M}C_{F}U_{M}G_{F}U_{M}U_{F}G_{M}C_{F}U_{M}G_{F}A_{M}G_{F}U_{M}A_{F}U_{M}C_{M}^{S}A_{M}^{S}UM-3′ (SEQ ID NO: 776), and the nucleotide sequence of the antisense strand is
5′-VP-A_{M}^{S}U_{F}^{S}G_{M}A_{F}U_{M}A_{F}C_{M}U_{F}C_{M}A_{F}G_{M}C_{F}A_{M}A_{F}C_{M}A_{F}G_{M}G_{F}A_{M}G_{F}G_{M}^{S}A_{M}^{S}G_{M}-OH-3′ (SEQ ID NO: 879), where a nucleotide followed by the subscript “F” is a 2′-fluoro nucleotide; a nucleotide followed by the subscript “M” is a 2′-O-methyl nucleotide; a nucleotide followed by the subscript “E” is a 2′-O-methoxyethyl nucleotide; the nucleobase of each “C_{E}” nucleotide is a 5-methylcytosine; each other “C” is a non-methylated cytosine; a superscript “S” is a phosphorothioate internucleotide linkage; and all other internucleotide linkages are phosphodiester internucleotide linkages. “5′-VP” is a 5′-VP modification at the 5′-terminal nucleotide of the antisense strand. “5′-OH” and “OH-3′” are hydroxyl moieties at the 5′-terminus and 3′ terminus, respectively.

In embodiments, a compound is DT-001255, where -L³-L⁴- is

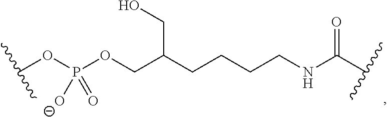 [SMILES: *OP(=O)([O-])OCC(CO)CCCCNC(*)=O]

the phosphate group of -L³-L⁴- is attached to the 3′ carbon of the 3′ terminal nucleotide of the sense strand, L⁶ is

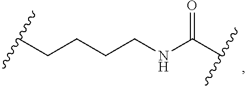 [SMILES: *CCCCNC(*)=O]

L⁵ is —NHC(O)—, R³ is hydrogen, R¹ is unsubstituted unbranched C₁₅ alkyl, R² is unsubstituted unbranched C₁₅ alkyl, the nucleotide sequence of the sense strand is
5′-OH-C_{M}^{S}C_{E}^{S}U_{E}C_{M}C_{F}U_{M}G_{F}U_{M}U_{F}G_{M}C_{F}U_{M}G_{F}A_{M}G_{F}U_{M}A_{F}U_{M}C_{M}^{S}A_{M}^{S}UM-3′ (SEQ ID NO: 777), and the nucleotide sequence of the antisense strand is
5′-VP-A_{M}^{S}U_{F}^{S}G_{M}A_{F}U_{M}A_{F}C_{M}U_{F}C_{M}A_{F}G_{M}C_{F}A_{M}A_{F}C_{M}A_{F}G_{M}G_{F}A_{M}G_{F}G_{M}^{S}A_{M}^{S}G_{M}-OH-3′ (SEQ ID NO: 879), where a nucleotide followed by the subscript “F” is a 2′-fluoro nucleotide; a nucleotide followed by the subscript “M” is a 2′-O-methyl nucleotide; a nucleotide followed by the subscript “E” is a 2′-O-methoxyethyl nucleotide; the nucleobase of each “C_{E}” nucleotide is a 5-methylcytosine; each other “C” is a non-methylated cytosine; the nucleobase of each “UE” nucleotide is a 5-methyluracil; each other “U” is a non-methylated uridine; a superscript “S” is a phosphorothioate internucleotide linkage; and all other internucleotide linkages are phosphodiester internucleotide linkages. “5′-VP” is a 5′-VP modification at the 5′-terminal nucleotide of the antisense strand. “5′-OH” and “OH-3′” are hydroxyl moieties at the 5′-terminus and 3′ terminus, respectively.

In embodiments, a compound is DT-001256, where -L³-L⁴- is

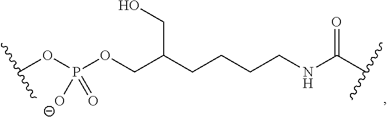 [SMILES: *OP(=O)([O-])OCC(CO)CCCCNC(*)=O]

the phosphate group of -L³-L⁴- is attached to the 3′ carbon of the 3′ terminal nucleotide of the sense strand, L⁶ is

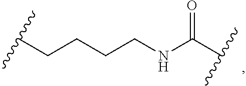 [SMILES: *CCCCNC(*)=O]

L⁵ is —NHC(O)—, R³ is hydrogen, R¹ is unsubstituted unbranched C₁₅ alkyl, R² is unsubstituted unbranched C₁₅ alkyl, the nucleotide sequence of the sense strand is
5′-OH-C_{M}^{S}C_{E}^{S}U_{E}C_{M}C_{F}U_{M}G_{F}U_{M}U_{F}G_{M}C_{F}U_{M}G_{F}A_{M}G_{F}U_{M}A_{F}U_{M}C_{E}^{S}A_{E}^{S}UM-3′ (SEQ ID NO: 778), and the nucleotide sequence of the antisense strand is
5′-VP-A_{M}^{S}U_{F}^{S}G_{M}A_{F}U_{M}A_{F}C_{M}U_{F}C_{M}A_{F}G_{M}C_{F}A_{M}A_{F}C_{M}A_{F}G_{M}G_{F}A_{M}G_{F}G_{M}^{S}A_{M}^{S}G_{M}-OH-3′ (SEQ ID NO: 879), where a nucleotide followed by the subscript “F” is a 2′-fluoro nucleotide; a nucleotide followed by the subscript “M” is a 2′-O-methyl nucleotide; a nucleotide followed by the subscript “E” is a 2′-O-methoxyethyl nucleotide; the nucleobase of each “C_{E}” nucleotide is a 5-methylcytosine; each other “C” is a non-methylated cytosine; the nucleobase of each “UE” nucleotide is a 5-methyluracil; each other “U” is a non-methylated uridine; a superscript “S” is a phosphorothioate internucleotide linkage; and all other internucleotide linkages are phosphodiester internucleotide linkages. “5′-VP” is a 5′-VP modification at the 5′-terminal nucleotide of the antisense strand. “5′-OH” and “OH-3′” are hydroxyl moieties at the 5′-terminus and 3′ terminus, respectively.

In embodiments, a compound is DT-001257, where -L³-L⁴- is

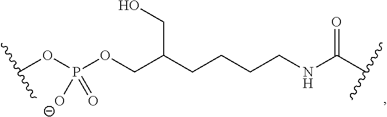 [SMILES: *OP(=O)([O-])OCC(CO)CCCCNC(*)=O]

the phosphate group of -L³-L⁴- is attached to the 3′ carbon of the 3′ terminal nucleotide of the sense strand, L⁶ is

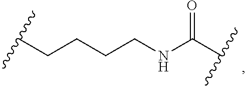 [SMILES: *CCCCNC(*)=O]

L⁵ is —NHC(O)—, R³ is hydrogen, R¹ is unsubstituted unbranched C₁₅ alkyl, R² is unsubstituted unbranched C₁₅ alkyl, the nucleotide sequence of the sense strand is
5′-OH-C_{E}^{S}C_{E}^{S}U_{E}C_{E}C_{F}U_{M}G_{F}U_{M}U_{F}G_{M}C_{F}U_{M}G_{F}A_{M}G_{F}U_{M}A_{F}U_{M}C_{M}^{S}A_{M}^{S}UM-3′ (SEQ ID NO: 779), and the nucleotide sequence of the antisense strand is
5′-VP-A_{M}^{S}U_{F}^{S}G_{M}A_{F}U_{M}A_{F}C_{M}U_{F}C_{M}A_{F}G_{M}C_{F}A_{M}A_{F}C_{M}A_{F}G_{M}G_{F}A_{M}G_{F}G_{M}^{S}A_{M}^{S}G_{M}-OH-3′ (SEQ ID NO: 879), where a nucleotide followed by the subscript “F” is a 2′-fluoro nucleotide; a nucleotide followed by the subscript “M” is a 2′-O-methyl nucleotide; a nucleotide followed by the subscript “E” is a 2′-O-methoxyethyl nucleotide; the nucleobase of each “C_{E}” nucleotide is a 5-methylcytosine; each other “C” is a non-methylated cytosine; the nucleobase of each “UE” nucleotide is a 5-methyluracil; each other “U” is a non-methylated uridine; a superscript “S” is a phosphorothioate internucleotide linkage; and all other internucleotide linkages are phosphodiester internucleotide linkages. “5′-VP” is a 5′-VP at the 5′-terminal nucleotide. “5′-OH” and “OH-3′” are hydroxyl moieties at the 5′-terminus and 3′ terminus, respectively.

In embodiments, a compound is DT-001858, where -L³-L⁴- is

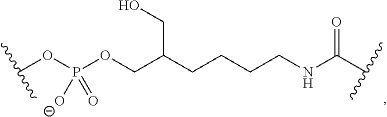 [SMILES: *OP(=O)([O-])OCC(CO)CCCCNC(*)=O]

the phosphate group of -L³-L⁴- is attached to the 3′ carbon of the 3′ terminal nucleotide of the sense strand, L⁶ is

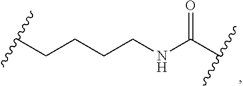 [SMILES: *CCCCNC(*)=O]

L⁵ is —NHC(O)—, R³ is hydrogen, R¹ is unsubstituted unbranched C₁₅ alkyl, R² is unsubstituted unbranched C₁₅ alkyl, the nucleotide sequence of the sense strand is
5′-OH-C_{M}^{S}C_{M}^{S}U_{M}C_{M}C_{M}U_{M}G_{F}U_{M}U_{F}G_{F}C_{F}U_{M}G_{M}A_{M}G_{M}U_{M}A_{M}U_{M}C_{M}A_{M}^{S}UM-3′ (SEQ ID NO: 887), and the nucleotide sequence of the antisense strand is
5′-VP-A_{M}^{S}U_{F}^{S}G_{M}A_{M}U_{M}A_{F}C_{M}U_{M}C_{M}A_{M}G_{M}C_{M}A_{M}A_{F}C_{M}A_{F}G_{M}G_{M}A_{M}G_{M}G_{M}^{S}A_{M}^{S}G_{M}-OH-3′ (SEQ ID NO: 902), where a nucleotide followed by the subscript “F” is a 2′-fluoro nucleotide; a nucleotide followed by the subscript “M” is a 2′-O-methyl nucleotide; a superscript “S” is a phosphorothioate internucleotide linkage; and all other internucleotide linkages are phosphodiester internucleotide linkages. “5′-VP” is a 5′-VP at the 5′-terminal nucleotide. “5′-OH” and “OH-3′” are hydroxyl moieties at the 5′-terminus and 3′ terminus, respectively.

In embodiments, a compound is DT-001859, where -L³-L⁴- is

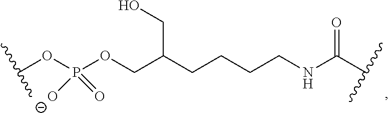 [SMILES: *OP(=O)([O-])OCC(CO)CCCCNC(*)=O]

the phosphate group of -L³-L⁴- is attached to the 3′ carbon of the 3′ terminal nucleotide of the sense strand, L⁶ is

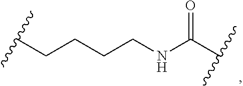 [SMILES: *CCCCNC(*)=O]

L⁵ is —NHC(O)—, R³ is hydrogen, R¹ is unsubstituted unbranched C₁₅ alkyl, R² is unsubstituted unbranched C₁₅ alkyl, the nucleotide sequence of the sense strand is
5′-OH-C_{M}^{S}C_{M}^{S}U_{F}C_{M}C_{M}U_{M}G_{F}U_{M}U_{F}G_{F}C_{F}U_{M}G_{M}A_{M}G_{M}U_{M}A_{M}U_{M}C_{M}^{S}A_{M}^{S}UM-3′ (SEQ ID NO: 878), and the nucleotide sequence of the antisense strand is
5′-VP-A_{M}^{S}U_{F}^{S}G_{M}A_{M}U_{M}A_{F}C_{M}U_{M}C_{M}A_{M}G_{M}C_{M}A_{M}A_{F}C_{M}A_{F}G_{M}G_{M}A_{M}G_{M}G_{M}^{S}A_{M}^{S}G_{M}-OH-3′ (SEQ ID NO: 902), where a nucleotide followed by the subscript “F” is a 2′-fluoro nucleotide; a nucleotide followed by the subscript “M” is a 2′-O-methyl nucleotide; a superscript “S” is a phosphorothioate internucleotide linkage; and all other internucleotide linkages are phosphodiester internucleotide linkages. “5′-VP” is a 5′-VP at the 5′-terminal nucleotide. “5′-OH” and “OH-3′” are hydroxyl moieties at the 5′-terminus and 3′ terminus, respectively.

In embodiments, a compound is DT-001860, where -L³-L⁴- is

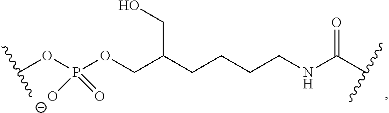 [SMILES: *OP(=O)([O-])OCC(CO)CCCCNC(*)=O]

the phosphate group of -L³-L⁴- is attached to the 3′ carbon of the 3′ terminal nucleotide of the sense strand, L⁶ is

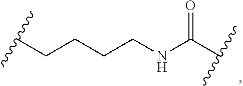 [SMILES: *CCCCNC(*)=O]

L⁵ is —NHC(O)—, R³ is hydrogen, R¹ is unsubstituted unbranched C₁₅ alkyl, R² is unsubstituted unbranched C₁₅ alkyl, the nucleotide sequence of the sense strand is
5′-HO-C_{M}^{S}C_{M}^{S}U_{M}C_{M}C_{M}U_{M}G_{F}U_{M}U_{F}G_{F}C_{F}U_{M}G_{M}A_{M}G_{M}U_{M}A_{M}U_{M}C_{M}^{S}A_{M}^{S}U_{M}- 3′ (SEQ ID NO: 774), and the nucleotide sequence of the antisense strand is
5′-VP-A_{M}^{S}U_{F}^{S}G_{M}A_{M}U_{M}A_{F}C_{M}U_{M}C_{M}A_{M}G_{M}C_{M}A_{M}A_{F}C_{M}A_{F}G_{M}G_{M}A_{M}G_{M}G_{M}^{S}A_{M}^{S}G_{E}-OH-3′ (SEQ ID NO: 975), where a nucleotide followed by the subscript “F” is a 2′-fluoro nucleotide; a nucleotide followed by the subscript “M” is a 2′-O-methyl nucleotide; a superscript “S” is a phosphorothioate internucleotide linkage; and all other internucleotide linkages are phosphodiester internucleotide linkages. “5′-VP” is a 5′-VP at the 5′-terminal nucleotide. “5′-OH” and “OH-3′” are hydroxyl moieties at the 5′-terminus and 3′ terminus, respectively.

In embodiments, -L³-L⁴- is

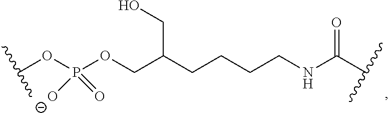 [SMILES: *OP(=O)([O-])OCC(CO)CCCCNC(*)=O]

the phosphate group of -L³-L⁴- is attached to the 3′ carbon of the 3′ terminal nucleotide of the sense strand, L⁶ is

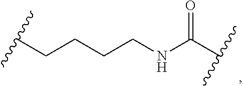 [SMILES: *CCCCNC(*)=O]

L⁵ is —NHC(O)—, R³ is hydrogen, R¹ is unsubstituted unbranched C₁₅ alkyl, R² is unsubstituted unbranched C₁₅ alkyl;
the nucleotide sequence of the sense strand is 5′-CCUCCUGUUGCUGAGUAUCAU-3′ (SEQ ID NO: 1018);
the nucleotide sequence of the antisense strand is 5′-AUGAUACUCAGCAACAGGAGGAG-3′ (SEQ ID NO: 1144);
the phosphate group at the 5′ terminus of the antisense strand is a 5′-VP; each nucleotide of the antisense strand is independently selected from a 2′-O-methyl nucleotide, a 2′-O-methoxyethyl nucleotide, and a 2′-fluoro nucleotide; each nucleotide of the sense strand is independently selected from 2′-O-methyl nucleotide, and a 2′-fluoro nucleotide;
at least one of the first two internucleotide linkages at the 5′ terminus of each strand is a phosphorothioate internucleotide linkage;
at least one of the last two internucleotide linkages at the 3′ terminus of each strand is a phosphorothioate internucleotide linkages;
and each other internucleotide linkage is a phosphodiester internucleotide linkage.

In embodiments, -L³-L⁴- is

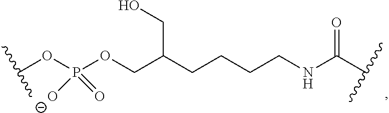 [SMILES: *OP(=O)([O-])OCC(CO)CCCCNC(*)=O]

the phosphate group of -L³-L⁴- is attached to the 3′ carbon of the 3′ terminal nucleotide of the sense strand, L⁶ is

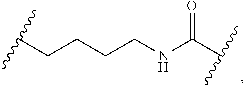 [SMILES: *CCCCNC(*)=O]

L⁵ is —NHC(O)—, R³ is hydrogen, R¹ is unsubstituted unbranched C₁₅ alkyl, R² is unsubstituted unbranched C₁₅ alkyl;
the nucleotide sequence of the sense strand is 5′-CCUCCUGUUGCUGAGUAUCAU-3′ (SEQ ID NO: 1018);
the nucleotide sequence of the antisense strand is 5′-AUGAUACUCAGCAACAGGAGGAG-3′ (SEQ ID NO: 1144);
the phosphate group at the 5′ terminus of the antisense strand is a 5′-VP;
each nucleotide of the antisense strand is independently selected from a 2′-O-methyl nucleotide, a 2′-O-methoxyethyl nucleotide, and a 2′-fluoro nucleotide;
each nucleotide of the sense strand is independently selected from 2′-O-methyl nucleotide, a 2′-O-methoxyethyl nucleotide, and a 2′-fluoro nucleotide;
at least one of the first two internucleotide linkages at the 5′ terminus of each strand is a phosphorothioate internucleotide linkage;
at least one of the last two internucleotide linkages at the 3′ terminus of each strand is a phosphorothioate internucleotide linkages;
and each other internucleotide linkage is a phosphodiester internucleotide linkage.

In embodiments, a ligand is a saturated or unsaturated C₅-C₂₀ alkyl. In embodiments, a ligand contains a saturated or unsaturated C₆-C₁₈ alkyl.

### Pharmaceutical Salts and Compositions

The compounds provided herein may be present as a pharmaceutical salt. In embodiments, the pharmaceutical salt is a sodium salt.

Pharmaceutically acceptable acid addition salts can be formed with inorganic acids and organic acids. Inorganic acids from which salts can be derived include, for example, hydrochloric acid, hydrobromic acid, sulfuric acid, nitric acid, phosphoric acid, and the like. Organic acids from which salts can be derived include, for example, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Pharmaceutically acceptable base addition salts can be formed with inorganic and organic bases. Inorganic bases from which salts can be derived include, for example, sodium, potassium, lithium, lithium, ammonium, calcium, magnesium, iron, zinc, copper, manganese, aluminum, and the like; particularly preferred are the ammonium, potassium, s16 odium, calcium and magnesium salts. Organic bases from which salts can be derived include, for example, primary, secondary, and tertiary amines, substituted amines including naturally occurring substituted amines, cyclic amines, basic ion exchange resins, and the like, specifically such as isopropylamine, trimethylamine, diethylamine, triethylamine, tripropylamine, and ethanolamine. Many such salts are known in the art, as described in WO 87/05297, Johnston et al., published Sep. 11, 1987 (incorporated by reference herein in its entirety).

In embodiments, a non-bridging heteroatom (e.g., an S— or O—) of a linkage of a compound provided herein may be protonated or associated with a counterion such as Na⁺, K⁺, etc. An acceptable salt (e.g. a pharmaceutically acceptable salt) of a compound may comprise fewer cationic counterions (such as Na⁺, K⁺, etc.) than there are non-bridging heteroatoms per molecule (i.e., some non-bridging heteroatoms are protonated and some are associated with counterions). In embodiments, a phosphate linkage attaching an -L³-L⁴- to a carbon of a nucleotide includes a non-bridging heteroatom. In embodiments, a phosphodiester linkage of a nucleic acid includes a non-bridging heteroatom. In embodiments, a phosphorothioate linkage of a nucleic acid includes a non-bridging heteroatom.

The compounds provided herein may be present as a pharmaceutical composition comprising the compound and a pharmaceutically acceptable diluent. In embodiments, the compound is present in a pharmaceutically acceptable diluent. In embodiments, the pharmaceutically acceptable diluent is a sterile aqueous solution. In embodiments, the sterile aqueous solution is a sterile saline solution.

A pharmaceutical composition may be prepared so that it is compatible with the intended mode of administration of the compound. Routes of administration of compounds include intravenous, intradermal, subcutaneous, transdermal, intramuscular, topical, and ocular administration.

Pharmaceutical compositions may be prepared for ocular administration to the eye in the form of an injection. Pharmaceutical compositions suitable for injection include sterile aqueous solutions, including sterile saline solutions. Pharmaceutical compositions suitable for injection may also be a lyophilized compound that is subsequently reconstitute with a pharmaceutically acceptable diluent in preparation for injection.

Alternatively, pharmaceutical compositions may be prepared for ocular administration to the eye in the form of an ophthalmic suspension (i.e. eye drops). Additional pharmaceutical preparations suitable for ocular administration include emulsions, ointments, aqueous gels, nanomicelles, nanoparticles, liposomes, dendrimers, implants, contact lenses, nanosuspensions, microneedles, and in situ thermosensitive gels.

### Methods of Use

Provided herein is a method for inhibiting the expression of peripheral myelin protein 22 (PMP22) mRNA in a cell, comprising contacting a cell with a nucleic compound provided herein, thereby inhibiting the expression of peripheral myelin protein 22 (PMP22) in the cell. In embodiments, the cell is a peripheral nerve cell. In embodiments, the cell is in vivo. In embodiments, the cell is in vitro.

Provided herein is a method for inhibiting the expression of peripheral myelin protein 22 (PMP22) in a subject, comprising administering to the subject an effective amount of a compound or pharmaceutical composition provided herein. In embodiments, the expression of peripheral myelin protein 22 (PMP22) is inhibited in the subject. In embodiments, the expression of PMP22 mRNA is inhibited in a peripheral nerve of the subject. In embodiments, the peripheral nerve is one or more of a sciatic nerve, a brachial plexus nerve, a tibial nerve, a peroneal nerve, a femoral nerve, a lateral femoral cutaneous nerve, and a spinal accessory nerve.

Provided herein is a method for increasing myelination and/or slowing the loss of myelination in a subject, comprising administering to the subject an effective amount of a compound or pharmaceutical composition provided herein. In embodiments, the administering increases myelination in the subject. In embodiments, the administering slows the loss of myelination in the subject. In embodiments, the subject has a peripheral demyelinating disease. In embodiments, the peripheral demyelinating disease is Charcot-Marie-Tooth disease (CMT). In embodiments, the Charcot-Marie-Tooth disease is Charcot-Marie-Tooth disease Type TA (CMT1A). In embodiments, the Charcot-Marie-Tooth disease Type 1E (CMT1E).

Provided herein is a method for treating Charcot-Marie-Tooth disease (CMT) in a subject in need thereof, comprising administering to the subject an effective amount compound or pharmaceutical composition provided herein. In embodiments, the Charcot-Marie-Tooth disease (CMT) is Charcot-Marie-Tooth disease Type 1A (CMT1A).

Provided herein is a method for treating Charcot-Marie-Tooth disease Type 1A (CMT1A) in a subject in need thereof, comprising administering to the subject an effective amount compound or pharmaceutical composition provided herein. Provided herein is a method for slowing the progression of Charcot-Marie-Tooth Disease Type 1A (CMT1A) in a subject in need thereof, comprising administering to the subject a compound or pharmaceutical composition provided herein.

In embodiments, the subject has Charcot-Marie-Tooth Disease Type 1A (CMT1A). CMT1A may be diagnosed by a medical professional using one or more routinely available assessments, including family history, medical history, and neurological examination. In embodiments, a subject is diagnosed as having CMT1A by the presence of one or more clinical indicators of CMT1A selected from: a family history of CMT1A; amplification of the PMP22 gene; distal muscle weakness; distal musculature atrophy, decreased deep tendon reflexes, distal sensory impairment; decreased compound muscle action potential; and decreased nerve conduction velocity.

Provided herein is a method for delaying the onset of CMT1A in a subject at risk for developing CMT1A, comprising administering to the subject a compound provided herein. A subject at risk for developing CMT1A may be identified by a medical professional using one or more routinely available assessments, including family history, medical history, and neurological examination. In embodiments, a subject is identified as beign at risk for developing CMT1A by the presence of one or more clinical indicators of CMT1A selected from: a family history of CMT1A; amplification of the PMP22 gene; distal muscle weakness; distal musculature atrophy; decreased deep tendon reflexes; distal sensory impairment; decreased compound muscle action potential; and decreased nerve conduction velocity.

In embodiments, a subject has a family history of CMT1A. In embodiments, amplification of the PMP22 gene in the subject is confirmed by genetic testing.

In embodiments, a subject has distal muscle weakness. In embodiments, the distal muscle weakness is in one or more of the arms, legs, hands and feet. In embodiments, the distal muscle weakness is measured by quantified muscular testing (QMT). In embodiments, the distal muscle weakness is reduced hand grip strength. In embodiments, the distal muscle weakness is reduced foot dorsiflexion.

In embodiments, a subject has distal musculature atrophy. In embodiments, the distal musculature atrophy is in one or more of the arms, legs, hands, and feet. In embodiments, the distal musculature atrophy is calf muscle atrophy.

In embodiments, a subject has reduced deep tendon reflexes.

In embodiments, a subject has distal sensory impairment.

In embodiments, the subject has reduced nerve conduction velocity (NCV). In embodiments, the nerve conduction velocity is motor nerve conduction velocity (MNCV). In embodiments, the nerve conduction velocity is sensory nerve conduction velocity (SNCV). Nerve conduction velocity may be determined by an electroneuroagraphy, i.e. a nerve conduction study, involving the placement of electrodes on the skin over a muscle or nerve. These electrodes produce a small electric impulse that stimulates nerves and allows for quantification of electrical activity from a distal muscle or nerve (those in the hands, lower arms, lower legs, and feet).

In embodiments, a subject has reduced compound muscle action potential (CMAP). CMAP may be determined by electromyography (EMG), a procedure which involves inserting a needle electrode through the skin to the muscle and measuring the bioelectrical activity of muscles, specific abnormalities in which indicate axon loss. EMG may be useful in further characterizing the distribution, activity, and severity of peripheral nerve involvement in CMT1A.

In embodiments, a subject has increased calf muscle fat fraction. In embodiments, calf muscle fat fraction is measured by magnetic resonance imaging (MRI).

In embodiments, a subject has elevated plasma neurofilament light (NfL) protein. In embodiments, a subject has elevated plasma transmembrane protease serine 5 (TMPRSS55).

In embodiments, the administration of the compound or pharmaceutical composition to the subject improves and/or slows the progression of one or more clinical indicators of Charcot-Marie-Tooth disease Type 1A in the subject. In embodiments, administration of the compound or pharmaceutical composition to the subject improves one or more clinical indicators of Charcot-Marie-Tooth disease Type 1A in the subject. In embodiments, administration of the compound or pharmaceutical composition to the subject slows the progression of one or more clinical indicators of Charcot-Marie-Tooth disease Type 1A in the subject. In embodiments, the one or more clinical indicator is selected from distal muscle weakness; distal sensory impairment; reduced nerve conduction velocity; reduced compound muscle action potential; reduced sensory nerve action potential; increased calf muscle fat fraction; elevated plasma neurofilament light (NfL); and elevated plasma transmembrane protease serine 5 (TMPRSS55). In embodiments, administration of the compound or pharmaceutical composition to the subject improves distal muscle weakness. In embodiments, administration of the compound slows the progression of distal muscle weakness. In embodiments, the distal muscle weakness is reduced hand grip strength. In embodiments, the distal muscle weakness is reduced foot dorsiflexion. In embodiments, administration of the compound or pharmaceutical composition improves distal sensory impairment. In embodiments, administration of the compound or pharmaceutical composition slows the progress of distal sensory impairment. In embodiments, administration of the compound or pharmaceutical composition increases nerve conduction velocity. In embodiments, administration of the compound or pharmaceutical composition slows the progression of reduced nerve conduction velocity. In embodiments, the nerve conduction velocity is motor nerve conduction velocity. In embodiments, the nerve condition velocity is sensory nerve conduction velocity. In embodiments, administration of the compound or pharmaceutical composition improves compound muscle action potential. In embodiments, administration of the compound slows the progression of reduced compound muscle action potential. In embodiments, administration of the compound or pharmaceutical composition improves sensory nerve action potential. In embodiments, administration of the compound or pharmaceutical composition slows the progression of reduced sensory nerve action potential. In embodiments, administration of the compound or pharmaceutical composition improves increased fat muscle fat fraction. In embodiments, administration of the compound or pharmaceutical composition slows the progression of increased fat muscle fat fraction. In embodiments, administration of the compound or pharmaceutical composition improves elevated plasma neurofilament light (NfL). In embodiments, administration of the compound or pharmaceutical composition slows the progression of elevated plasma neurofilament light (NfL). In embodiments, administration of the compound or pharmaceutical composition improves elevated plasma transmembrane protease serine 5 (TMPRSS55). In embodiments, administration of the compound or pharmaceutical composition slows the progression of elevated plasma transmembrane protease serine 5 (TMPRSS55).

Disease severity and disease progression in subjects may be determined using one or more clinical assessments. In embodiments, disease severity in a subject is determined by performing one or more clinical assessments. In embodiments, disease progression in a subject is determined by performing one or more clinical assessments. In embodiments, disease progression is determined by measuring the change over time in one or more clinical assessments. In embodiments, the clinical assessment is selected from the Charcot-Marie-Tooth Neuropathy Score (CMTNS), the Charcot-Marie-Tooth Neuropathy Score with Rasch weighting (CMTNS-R), the Charcot Marie-Tooth Neuropathy Score Version 2 (CMTNS-v2), the Charcot-Marie-Tooth Examination Score (CMTES), the Charcot-Marie-Tooth Examination Score with Rasch weighting (CMTES-R), the Charcot-Marie-Tooth Functional Outcome Measure (CMT-FOM), the Charcot-Marie-Tooth Disease Pediatric Scale, the Charcot-Marie-Tooth Disease Infant Scale, the Charcot-Marie-Tooth Health Index, and the Overall Neuropathy Limitation Scale (ONLS). In embodiments, the clinical assessment is the Charcot-Marie-Tooth Neuropathy Score (CMTNS). In embodiments, the clinical assessment is the Charcot-Marie-Tooth Neuropathy Score with Rasch weighting (CMTNS-R). In embodiments, the clinical assessment is the Charcot Marie-Tooth Neuropathy Score Version 2 (CMTNS-v2). In embodiments, the clinical assessment is the Charcot-Marie-Tooth Examination Score (CMTES). In embodiments, the clinical assessment is the Charcot-Marie-Tooth Examination Score with Rasch weighting (CMTES-R). In embodiments, the clinical assessment is the Charcot-Marie-Tooth Functional Outcome Measure (CMT-FOM). In embodiments, the clinical assessment is the Charcot-Marie-Tooth Disease Pediatric Scale. In embodiments, the clinical assessment is the Charcot-Marie-Tooth Disease Infant Scale. In embodiments, the clinical assessment the Charcot-Marie-Tooth Health Index. In embodiments, the clinical assessment is and the Overall Neuropathy Limitation Scale (ONLS).

In embodiments, administration is intravenous administration. In embodiments, the administration is subcutaneous administration.

In embodiments, at least one additional therapy is administered to the subject. In embodiments, the at least one additional therapy is PXT3003 comprising baclofen, sorbitol, and naltrexone.

In embodiments, compounds provided herein are for use in therapy. In embodiments, pharmaceutical compositions provided herein are for use in therapy. In embodiments, the therapy is the treatment of a demyelinating disease. In embodiments, the therapy is the treatment of Charcot-Marie-Tooth disease. In embodiments, the therapy is the treatment of Charcot-Marie-Tooth disease Type 1A (CMT1A).

### Formulations

Various formulations are available to facilitate compound use both in vitro and as therapeutic agents. Accordingly, in embodiments, a compound provided herein is present in a formulation.

Compounds may be formulated with cationic lipids to facilitate transfection into cells. Suitable cationic lipid reagents for transfection include Lipofectamine reagents, such as Lipofectamine RNAiMAX.

For use in vivo as therapeutic agents, nucleic acids compounds may be encapsulated into lipid nanoparticles. Lipid nanoparticles generally comprise a cationic lipid, a non-cationic lipid, and a lipid that prevents aggregation of the nanoparticle. Suitable cationic lipids include DLin-MC3-DMA ((6Z,9Z,28Z,31Z)-Heptatriaconta-6,9,28,31-tetraen-19-yl 4-(dimethylamino)butanoate), DLin-KC2-DMA (2,2-dilinoleyl-4-dimethylaminoethyl-[1,3]-dioxolane) and the lipidoid C12-200. Suitable non-cationic lipids include, for example, DOPC (1,2-dioleoyl-sn-glycero-3-phosphatidylcholine) and DSPC (1,2-distearoyl-sn-glycero-3-phosphocholine). Examples of lipids that prevent aggregation include, for example, polyethylene glycol (PEG)-lipids, such as PEG-C-DMA (3-N—[(ω-methoxypoly(ethylene glycol)2000)carbamoyl]-1,2-dimyristyloxy-propylamine), PEG2000-C-DMG (α-(3-{[1,2-di(myristyloxy)proponoxy]carbonylamino}propyl)-ω-methoxy, polyoxyethylene), and mPEG-DSPE (1,2-distearoyl-sn-glycero-3-phosphoethanolamine-N-[methoxy(polyethylene glycol)-2000]).

### Embodiments

Embodiment 1. A compound comprising an antisense strand and a sense strand hybridized to form a double-stranded nucleic acid, wherein each of the antisense strand and sense strands is 15 to 25 nucleotides in length, the nucleotide sequence of the antisense strand is at least 90% complementary to the human peripheral myelin protein 22 mRNA (SEQ ID NO: 1170), and the nucleotide sequence of the sense strand has no more than two mismatches to the nucleotide sequence of the antisense strand in the double-stranded region.
Embodiment 2. The compound of embodiment 1, wherein each of the antisense strand and sense strands is 15 to 25 nucleotides in length, the nucleotide sequence of the antisense strand comprises at least 15 contiguous nucleotides of any one of SEQ ID NOs 491, 492, 493, 494,495,497,498,503,504,506,510,511,514,515,516,518,524,526,529,531,532, 533, 534, 535, 536, 538, 539, 540, 541, 542, 543, 545, 546, 547, 548, 550, 553, 554, 556, 558, 559, 560, 561, 563, 567, 569, 575, 576, 579, 580, 581, 582, 583, 585, 590, 591, 595, 597, 600, 605, 609, 610, 618, 622, 623, 628, 630, 631, 633, 635, 637, 639, 641, 642, 643, 644, 645, 1112, 1113, 1114, 1115, 1116, 1117, 1118, 1119, 1120, 1122, 1124, 1125, 1126, 1127, 1128, 1129, 1130, 1131, 1132, 1133, 1134, 1135, 1136, 1137, 1138, 1139, 1140, 1141, 1142, 1143, 1144, 1145, 1146, 1147, 1148, 1149, 1150, 1151, 1152, 1153, 1154, 1155, 1156, 1157, 1158, 1159, 1160, 1161, 1162, 1163, 1164, 1165, 1166, 1167, 1168, 1169, 1118, 1121, 1123, 1126, and 1144, and the nucleotide sequence of the sense strand has no more than two mismatches to the nucleotide sequence of the antisense strand.
Embodiment 3. The compound of embodiment 2, wherein the nucleotide sequence of the antisense strand comprises at least 16, at least 17, at least 18, at least 19, at least 20, at least 21, at least 22, or 23 contiguous nucleotides selected from any one of SEQ ID NOs 491, 492, 493, 494, 495, 497, 498, 503, 504, 506, 510, 511, 514, 515, 516, 518, 524, 526, 529, 531, 532, 533, 534, 535, 536, 538, 539, 540, 541, 542, 543, 545, 546, 547, 548, 550, 553, 554, 556, 558, 559, 560, 561, 563, 567, 569, 575, 576, 579, 580, 581, 582, 583, 585, 590, 591, 595, 597, 600, 605, 609, 610, 618, 622, 623, 628, 630, 631, 633, 635, 637, 639, 641, 642, 643, 644, 645, 1112, 1113, 1114, 1115, 1116, 1117, 1118, 1119, 1120, 1122, 1124, 1125, 1126, 1127, 1128, 1129, 1130, 1131, 1132, 1133, 1134, 1135, 1136, 1137, 1138, 1139, 1140, 1141, 1142, 1143, 1144, 1145, 1146, 1147, 1148, 1149, 1150, 1151, 1152, 1153, 1154, 1155, 1156, 1157, 1158, 1159, 1160, 1161, 1162, 1163, 1164, 1165, 1166, 1167, 1168, 1169, 1118, 1121, 1123, 1126, and 1144.
Embodiment 4. The compound of embodiment 3, wherein the nucleotide sequence of the antisense strand comprises 19 contiguous nucleotides of a nucleotide sequence selected from any one of SEQ ID NOs 491, 492, 493, 494, 495, 497, 498, 503, 504, 506, 510, 511, 514, 515, 516, 518, 524, 526, 529, 531, 532, 533, 534, 535, 536, 538, 539, 540, 541, 542, 543, 545, 546, 547, 548, 550, 553, 554, 556, 558, 559, 560, 561, 563, 567, 569, 575, 576, 579, 580, 581, 582, 583, 585, 590, 591, 595, 597, 600, 605, 609, 610, 618, 622, 623, 628, 630, 631, 633, 635, 637, 639, 641, 642, 643, 644, 645, 1112, 1113, 1114, 1115, 1116, 1117, 1118, 1119, 1120, 1122, 1124, 1125, 1126, 1127, 1128, 1129, 1130, 1131, 1132, 1133, 1134, 1135, 1136, 1137, 1138, 1139, 1140, 1141, 1142, 1143, 1144, 1145, 1146, 1147, 1148, 1149, 1150, 1151, 1152, 1153, 1154, 1155, 1156, 1157, 1158, 1159, 1160, 1161, 1162, 1163, 1164, 1165, 1166, 1167, 1168, 1169, 1118, 1121, 1123, 1126, and 1144.
Embodiment 5. The compound of any one of embodiments 1 to 4, wherein the antisense strand is 17 to 23 nucleotides in length.
Embodiment 6. The compound of any one of embodiments 1 to 5, wherein the antisense strand is 19 to 21 nucleotides in length.
Embodiment 7. The compound of any one of embodiments 1 to 5, wherein the antisense strand is 21 to 23 nucleotides in length.
Embodiment 8. The compound of any one of embodiments 1 to 5, wherein the antisense strand is 19 nucleotides in length.
Embodiment 9. The compound of any one of embodiments 1 to 5, wherein the antisense strand is 20 nucleotides in length.
Embodiment 10. The compound of any one of embodiments 1 to 5, wherein the antisense strand is 21 nucleotides in length.
Embodiment 11. The compound of any one of embodiments 1 to 5, wherein the antisense strand is 22 nucleotides in length.
Embodiment 12. The compound of any one of embodiments 1 to 5, wherein the antisense strand is 23 nucleotides in length.
Embodiment 13. The compound of any one of embodiments 1 to 12, wherein the nucleotide sequence of the antisense strand is at least 95% complementary to SEQ ID NO: 1.
Embodiment 14. The compound of any one of embodiments 1 to 12, wherein the nucleotide sequence of the antisense strand is 100% complementary to SEQ ID NO: 1.
Embodiment 15. The compound of any one of embodiments 1 to 14, wherein the sense strand is 17 to 23 nucleotides in length.
Embodiment 16. The compound of any one of embodiments 1 to 14, wherein the sense strand is 19 to 21 nucleotides in length.
Embodiment 17. The compound of any one of embodiments 1 to 14, wherein the sense strand is 21 to 23 nucleotides in length.
Embodiment 18. The compound of any one of embodiments 1 to 14, wherein the sense strand is 19 nucleotides in length.
Embodiment 19. The compound of any one of embodiments 1 to 14, wherein the sense strand is 20 nucleotides in length.
Embodiment 20. The compound of any one of embodiments 1 to 14, wherein the sense strand is 21 nucleotides in length.
Embodiment 21. The compound of any one of embodiments 1 to 14, wherein the sense strand is 22 nucleotides in length.
Embodiment 22. The compound of any one of embodiments 1 to 14, wherein the sense strand is 23 nucleotides in length.
Embodiment 23. The compound of any one of embodiments 1 to 22, wherein the double-stranded region is 15 to 25 nucleotide pairs in length.
Embodiment 24. The compound of any one of embodiments 1 to 22, wherein the double-stranded region is 17 to 23 nucleotide pairs in length.
Embodiment 25. The compound of any one of embodiments 1 to 22, wherein the double-stranded region is 19 to 21 nucleotide pairs in length.
Embodiment 26. The compound of any one of embodiments 1 to 22, wherein the double-stranded region is 19 nucleotide pairs in length.
Embodiment 27. The compound of any one of embodiments 1 to 22, wherein the double-stranded region is 20 nucleotide pairs in length.
Embodiment 28. The compound of any one of embodiments 1 to 22, wherein the double-stranded region is 21 nucleotide pairs in length.
Embodiment 29. The compound of any one of embodiments 1 to 28, wherein the nucleotide sequence of the sense strand has no more than one mismatch to the nucleotide sequence of the antisense strand in the double-stranded region.
Embodiment 30. The compound of any one of embodiments 1 to 28, wherein the nucleotide sequence of the sense strand has no mismatches to the nucleotide sequence of the antisense strand in the double-stranded region.
Embodiment 31. The compound of embodiment 4, wherein the antisense strand is 21 nucleotides in length and the nucleotide sequence of the antisense strand is identical to a nucleotide sequence selected from any one of SEQ ID NOs 491, 492, 493, 494, 495, 497, 498, 503, 504, 506, 510, 511, 514, 515, 516, 518, 524, 526, 529, 531, 532, 533, 534, 535, 536, 538, 539, 540, 541, 542, 543, 545, 546, 547, 548, 550, 553, 554, 556, 558, 559, 560, 561, 563, 567, 569, 575, 576, 579, 580, 581, 582, 583, 585, 590, 591, 595, 597, 600, 605, 609, 610, 618, 622, 623, 628, 630, 631, 633, 635, 637, 639, 641, 642, 643, 644, and 645.
Embodiment 32. The compound of embodiment 4, wherein the antisense strand is 23 nucleotides in length and the nucleotide sequence of the antisense strand is identical to a nucleotide sequence selected from any one of SEQ ID NOs 1112, 1113, 1114, 1115, 1116, 1117, 1118, 1119, 1120, 1122, 1124, 1125, 1126, 1127, 1128, 1129, 1130, 1131, 1132, 1133, 1134, 1135, 1136, 1137, 1138, 1139, 1140, 1141, 1142, 1143, 1144, 1145, 1146, 1147, 1148, 1149, 1150, 1151, 1152, 1153, 1154, 1155, 1156, 1157, 1158, 1159, 1160, 1161, 1162, 1163, 1164, 1165, 1166, 1167, 1168, 1169, 1118, 1126, and 1144.
Embodiment 33. The compound of any one of embodiments 1 to 32, wherein the antisense strand and the sense strand are not covalently linked.
Embodiment 34. The compound of any one of embodiments 1 to 33, wherein the hybridization of the antisense strand to the sense strand forms at least one blunt end.
Embodiment 35. The compound of embodiment 34, wherein the hybridization of the antisense strand to the sense strand forms a blunt end at each terminus of the compound.
Embodiment 36. The compound of any one of embodiments 1 to 34, wherein at least one strand comprises a 3′ nucleotide overhang of one to five nucleotides.
Embodiment 37. The compound of embodiment 36, wherein the sense strand comprises the 3′ nucleotide overhang.
Embodiment 38. The compound of embodiment 36, wherein the antisense strand comprises the 3′ nucleotide overhang.
Embodiment 39. The compound of embodiment 36, wherein each of the sense strand and the antisense strand comprises a 3′ nucleotide overhang of one to five nucleotides.
Embodiment 40. The compound of embodiment 38 or 39, wherein each nucleotide of the 3′ nucleotide overhang of the antisense strand is complementary to SEQ ID NO: 1.
Embodiment 41. The compound of embodiment 38 or 39, wherein each nucleotide of the 3′ nucleotide overhang of the antisense strand is not complementary to SEQ ID NO: 1.
Embodiment 42. The compound of any one of embodiments 36 to 41, wherein each nucleotide of the 3′ nucleotide overhang is a deoxythymidine.
Embodiment 43. The compound of any one of embodiments 36 to 42, wherein the 3′ nucleotide overhang is two nucleotides in length.
Embodiment 44. The compound of any one of embodiments 1 to 4, wherein the double-stranded nucleic acid comprises an antisense strand and sense strand of any of the following pairs of SEQ ID NOs: 1018 and 1144; SEQ ID NOs: 1018 and 1144; SEQ ID NOs: 993 and 1164; SEQ ID NOs: 1108 and 1156; SEQ ID NOs: 1051 and 1158; SEQ ID NOs: 1069 and 1168; SEQ ID NOs: 993 and 1164; SEQ ID NOs: 1108 and 1156; SEQ ID NOs: 1047 and 1160; SEQ ID NOs: 1111 and 1161; SEQ ID NOs: 1066 and 1136; SEQ ID NOs: 1110 and 1122; SEQ ID NOs: 986 and 1142; SEQ ID NOs: 1047 and 1160; SEQ ID NOs: 1111 and 1161; SEQ ID NOs: 1066 and 1136; SEQ ID NOs: 1110 and 1122; SEQ ID NOs: 986 and 1142; SEQ ID NOs: 1018 and 1144; SEQ ID NOs: 1018 and 1144; SEQ ID NOs: 1018 and 1144; SEQ ID NOs: 1018 and 1144; SEQ ID NOs: 1018 and 1144; SEQ ID NOs: 1018 and 1144; SEQ ID NOs: 1018 and 1144; SEQ ID NOs: 1015 and 1144; SEQ ID NOs: 1015 and 1144; SEQ ID NOs: 1015 and 1144; SEQ ID NOs: 1091 and 1151; SEQ ID NOs: 1045 and 1152; SEQ ID NOs: 1103 and 1155; SEQ ID NOs: 1065 and 1140; SEQ ID NOs: 1067 and 1141; SEQ ID NOs: 1021 and 1147; SEQ ID NOs: 1019 and 1143; SEQ ID NOs: 1000 and 1127; SEQ ID NOs: 1060 and 1138; SEQ ID NOs: 1034 and 1153; SEQ ID NOs: 1088 and 1157; SEQ ID NOs: 1037 and 1154; SEQ ID NOs: 1091 and 1151; SEQ ID NOs: 1045 and 1152; SEQ ID NOs: 1103 and 1155; SEQ ID NOs: 1054 and 1126; SEQ ID NOs: 1028 and 1131; SEQ ID NOs: 1097 and 1128; SEQ ID NOs: 1065 and 1140; SEQ ID NOs: 1001 and 1129; SEQ ID NOs: 994 and 1112; SEQ ID NOs: 1086 and 1145; SEQ ID NOs: 977 and 1125; SEQ ID NOs: 1067 and 1141; SEQ ID NOs: 1021 and 1147; SEQ ID NOs: 1077 and 1134; SEQ ID NOs: 1022 and 1117; SEQ ID NOs: 1010 and 1165; SEQ ID NOs: 1071 and 1133; SEQ ID NOs: 1009 and 1150; SEQ ID NOs: 1081 and 1119; SEQ ID NOs: 997 and 1124; SEQ ID NOs: 1063 and 1130; SEQ ID NOs: 1029 and 1148; SEQ ID NOs: 1056 and 1163; SEQ ID NOs: 1039 and 1113; SEQ ID NOs: 1033 and 1149; SEQ ID NOs: 1031 and 1132; SEQ ID NOs: 1008 and 1139; SEQ ID NOs: 1026 and 1118; SEQ ID NOs: 999 and 1166; SEQ ID NOs: 979 and 1169; SEQ ID NOs: 1098 and 1137; SEQ ID NOs: 1027 and 1135; SEQ ID NOs: 1073 and 1114; SEQ ID NOs: 1078 and 1116; SEQ ID NOs: 981 and 1115; SEQ ID NOs: 1030 and 1159; SEQ ID NOs: 992 and 1146; SEQ ID NOs: 1024 and 1167; SEQ ID NOs: 1007 and 1162; SEQ ID NOs: 978 and 1120; SEQ ID NOs: 1028 and 1131; SEQ ID NOs: 1097 and 1128; SEQ ID NOs: 994 and 1112; SEQ ID NOs: 1086 and 1145; SEQ ID NOs: 977 and 1125; SEQ ID NOs: 1022 and 1117; SEQ ID NOs: 1010 and 1165; SEQ ID NOs: 1071 and 1133; SEQ ID NOs: 1009 and 1150; SEQ ID NOs: 1081 and 1119; SEQ ID NOs: 1029 and 1148; and SEQ ID NOs: 1039 and 1113.
Embodiment 45. The compound of any one of embodiments 1 to 44, wherein at least one nucleotide of the antisense strand is a modified nucleotide.
Embodiment 46. The compound of any one of embodiments 1 to 45, wherein at least one nucleotide of the sense strand is a modified nucleotide.
Embodiment 47. The compound of any one of embodiments 1 to 46, wherein each nucleotide of the antisense strand forming the double-stranded region is a modified nucleotide.
Embodiment 48. The compound of any one of embodiments 1 to 47, wherein each nucleotide of the sense strand forming the double-stranded region is a modified nucleotide.
Embodiment 49. The compound of any one of embodiments 1 to 48, wherein each nucleotide of the antisense strand is a modified nucleotide.
Embodiment 50. The compound of any one of embodiments 1 to 49, wherein each nucleotide of the sense strand is a modified nucleotide.
Embodiment 51. The compound of any one of embodiments 45 to 50, wherein the modified nucleotide comprises one or more of a modified sugar moiety, a modified internucleotide linkage, and a 5′-terminal modified phosphate group.
Embodiment 52. The compound of embodiment 51, wherein the modified nucleotide comprising a modified sugar moiety is selected from a 2′-fluoro nucleotide, a 2′-O-methyl nucleotide, a 2′-O-methoxyethyl nucleotide, and a bicyclic sugar nucleotide.
Embodiment 53. The compound of embodiment 51, wherein the modified internucleotide linkage is a phosphorothioate internucleotide linkage.
Embodiment 54. The compound of embodiment 53, wherein the first two internucleotide linkages at the 5′ terminus of the sense strand and the last two internucleotide linkages at the 3′ terminus of the sense strand are phosphorothioate internucleotide linkages.
Embodiment 55. The compound of embodiment 54, wherein the first two internucleotide linkages at the 5′ terminus of the antisense strand and the last two internucleotide linkages at the 3′ terminus of the antisense strand are phosphorothioate internucleotide linkages.
Embodiment 56. The compound of embodiment 52, wherein the covalent linkage of the bicyclic sugar is selected from a 4′-CH(CH₃)—O-2′ linkage, a 4′-(CH₂)₂—O-2′ linkage, a 4′-CH(CH₂—OMe)-O-2′ linkage, 4′-CH₂—N(CH₃)—O-2′ linkage, and 4′-CH₂—N(H)—O-2′ linkage.
Embodiment 57. The compound of embodiment 51, wherein the 5′-terminal modified phosphate group is a 5′-(E)-vinylphosphonate.
Embodiment 58. The compound of any one of embodiments 1 to 57, wherein the antisense strand is 21 nucleotides in length and the nucleotides of the antisense strand are modified such that, counting from the 5′ terminus of the antisense strand, nucleotides 1, 3, 5, 7, 9, 11, 13, 15, 17, and 19 are 2′-O-methyl nucleotides, nucleotides 2, 4, 6, 8, 10, 12, 14, 16, and 18 are 2′-fluoro nucleotides, and nucleotides 20 and 21 are beta-D-deoxynucleotides, the first two internucleotide linkages at the 5′ terminus and the last two internucleotide linkages at the 3′ terminus are phosphorothioate internucleotide linkages, and each other internucleotide linkage is a phosphodiester internucleotide linkage; and wherein the sense strand is 21 nucleotides in length and the nucleotides of the sense strand are modified such that, counting from the 5′ terminus of the sense strand, nucleotides 1, 3, 5, 7, 9, 11, 13, 15, 17, and 19 are 2′-fluoro nucleotides, nucleotides 2, 4, 6, 8, 10, 12, 14, 16, and 18 are 2′-O-methyl nucleotides, and nucleotides 20 and 21 are beta-D-deoxynucleotides, the first two internucleotide linkages at the 5′ terminus and the last two internucleotide linkages at the 3′ terminus are phosphorothioate internucleotide linkages, and each other internucleotide linkage is a phosphodiester internucleotide linkage.
Embodiment 59. The compound of any one of embodiments 1 to 57, wherein the antisense strand is 21 nucleotides in length and the nucleotides of the antisense strand are modified such that, counting from the 5′ terminus of the antisense strand, nucleotides 1, 3, 5, 7, 9, 11, 13, 15, 17, and 19 are 2′-O-methyl nucleotides, nucleotides 2, 4, 6, 8, 10, 12, 14, 16, and 18 are 2′-fluoro nucleotides, and nucleotides 20 and 21 are beta-D-deoxy nucleotides, the first two internucleotide linkages at the 5′ terminus and the last two internucleotide linkages at the 3′ terminus are phosphorothioate internucleotide linkages, and each other internucleotide linkage is a phosphodiester internucleotide linkage; and wherein the sense strand is 19 nucleotides in length and the nucleotides of the sense strand are modified such that, counting from the 5′ terminus of the sense strand, nucleotides 1, 3, 5, 7, 9, 11, 13, 15, 17, and 19 are 2′-fluoro nucleotides and nucleotides 2, 4, 6, 8, 10, 12, 14, 16, and 18 are 2′-O-methyl nucleotides, the first two internucleotide linkages at the 5′ terminus and the last two internucleotide linkages at the 3′ terminus are phosphorothioate internucleotide linkages, and each other internucleotide linkage is a phosphodiester internucleotide linkage.
Embodiment 60. The compound of any one of embodiments 1 to 57, wherein the antisense strand is 23 nucleotides in length and wherein the nucleotides of the antisense strand are modified such that, counting from the 5′ terminus of the antisense strand, nucleotides 1, 3, 5, 7, 9, 11, 13, 15, 17, 19, 21, 22, and 23 are 2′-O-methyl nucleotides and nucleotides 2, 4, 6, 8, 10, 12, 14, 16, 18, and 20 are 2′-fluoro nucleotides the first two internucleotide linkages at the 5′ terminus and the last two internucleotide linkages at the 3′ terminus are phosphorothioate internucleotide linkages, and each other internucleotide linkage is a phosphodiester internucleotide linkage; and wherein the sense strand is 21 nucleotides in length and the nucleotides of the sense strand are modified such that, counting from the 5′ terminus of the sense strand, nucleotides 1, 3, 5, 7, 9, 11, 13, 15, 17, 19, and 21 are 2′-fluoro nucleotides, nucleotides 2, 4, 6, 8, 10, 12, 14, 16, 18, and 20 are 2′-O-methyl nucleotides, the first two internucleotide linkages at the 5′ terminus and the last two internucleotide linkages at the 3′ terminus are phosphorothioate internucleotide linkages, and each other internucleotide linkage is a phosphodiester internucleotide linkage.
Embodiment 61. The compound of any one of embodiments 1 to 57, wherein the antisense strand is 23 nucleotides in length and wherein the nucleotides of the antisense strand are modified such that, counting from the 5′ terminus of the antisense strand, nucleotides 1, 3, 5, 7, 9, 11, 13, 15, 17, 19, 21, 22, and 23 are 2′-O-methyl nucleotides, nucleotides 2, 4, 6, 8, 10, 12, 14, 16, 18, and 20 are 2′-fluoro nucleotides, the first two internucleotide linkages at the 5′ terminus and the last two internucleotide linkages at the 3′ terminus are phosphorothioate internucleotide linkages, and each other internucleotide linkage is a phosphodiester internucleotide linkage; and wherein the sense strand is 21 nucleotides in length and wherein the nucleotides of the sense strand are modified such that, counting from the 5′ terminus of the sense strand, nucleotides 1, 2, 3, 4, 6, 8, 12, 14, 16, 18, 19, 20, and 21 are 2′-O-methyl nucleotides, nucleotides 5, 7, 9, 10, 11, 13, 15, and 17 are 2′-fluoro nucleotides, the first two internucleotide linkages at the 5′ terminus and the last two internucleotide linkages at the 3′ terminus are phosphorothioate internucleotide linkages, and each other internucleotide linkage is a phosphodiester internucleotide linkage.
Embodiment 62. The compound of any one of embodiments 1 to 57, wherein the antisense strand is 23 nucleotides in length and wherein the nucleotides of the antisense strand are modified such that, counting from the 5′ terminus of the antisense strand, nucleotides 1, 3, 5, 7, 9, 11, 12, 13, 15, 17, 19, 21, 22, and 23 are 2′-O-methyl nucleotides and nucleotides 2, 4, 6, 8, 10, 14, 16, 18, and 20 are 2′-fluoro nucleotides, the first two internucleotide linkages at the 5′ terminus and the last two internucleotide linkages at the 3′ terminus are phosphorothioate internucleotide linkages, and each other internucleotide linkage is a phosphodiester internucleotide linkage; and wherein the sense strand is 21 nucleotides in length and the nucleotides of the sense strand are modified such that, counting from the 5′ terminus of the sense strand, nucleotides 1, 3, 5, 7, 9, 10, 11, 13, 15, 17, 19, and 21 are 2′-fluoronucleotides, nucleotides 2, 4, 6, 8, 12, 14, 16, 18, and 20 are 2′-O-methyl nucleotides, the first two internucleotide linkages at the 5′ terminus and the last two internucleotide linkages at the 3′ terminus are phosphorothioate internucleotide linkages, and each other internucleotide linkage is a phosphodiester internucleotide linkage.
Embodiment 63. The compound of any one of embodiments 1 to 57, wherein the antisense strand is 23 nucleotides in length and wherein the nucleotides of the antisense strand are modified such that, counting from the 5′ terminus of the antisense strand, nucleotides 1, 3, 5, 7, 9, 10, 11, 13, 15, 17, 19, 21, 22, and 23 are 2′-O-methyl nucleotides and nucleotides 2, 4, 6, 8, 12, 14, 16, 18, and 20 are 2′-fluoro nucleotides, the first two internucleotide linkages at the 5′ terminus and the last two internucleotide linkages at the 3′ terminus are phosphorothioate internucleotide linkages, and each other internucleotide linkage is a phosphodiester internucleotide linkage; and wherein the sense strand is 21 nucleotides in length and nucleotides of the sense strand are modified such that, counting from the 5′ terminus of the sense strand, nucleotides 1, 3, 5, 7, 9, 11, 12, 13, 15, 17, 19, and 21 are 2′-fluoro nucleotides, nucleotides 2, 4, 6, 8, 10, 14, 16, 18, and 20 are 2′-O-methyl nucleotides, the first two internucleotide linkages at the 5′ terminus and the last two internucleotide linkages at the 3′ terminus are phosphorothioate internucleotide linkages, and each other internucleotide linkage is a phosphodiester internucleotide linkage.
Embodiment 64. The compound of any one of embodiments 1 to 57, wherein the antisense strand is 23 nucleotides in length and the nucleotides of the antisense strand are modified such that, counting from the 5′ terminus of the antisense strand, nucleotides 1, 3, 5, 7, 8, 9, 11, 12, 13, 15, 17, 19, 21, 22, and 23 are 2′-O-methyl nucleotides, nucleotides 2, 4, 6, 10, 14, 16, 18, and 20 are 2′-fluoro nucleotides, the first two internucleotide linkages at the 5′ terminus and the last two internucleotide linkages at the 3′ terminus are phosphorothioate internucleotide linkages, and each other internucleotide linkage is a phosphodiester internucleotide linkage; and wherein the sense strand is 21 nucleotides in length and the nucleotides of the sense strand are modified such that, counting from the 5′ terminus of the sense strand, nucleotides 1, 2, 3, 4, 5, 6, 8, 10, 12, 14, 16, 18, 19, 20, and 21 are 2′-O-methyl nucleotides, nucleotides 7, 9, 11, 13, 15, and 17 are 2′-fluoro nucleotides, the first two internucleotide linkages at the 5′ terminus and the last two internucleotide linkages at the 3′ terminus are phosphorothioate internucleotide linkages, and each other internucleotide linkage is a phosphodiester internucleotide linkage.
Embodiment 65. The compound of any one of embodiments 1 to 57, wherein the antisense strand is 23 nucleotides in length and the nucleotides of the antisense strand are modified such that, counting from the 5′ terminus of the antisense strand, nucleotides 1, 3, 4, 5, 7, 8, 9, 10, 11, 12, 13, 15, 17, 18, 19, 20, 21, 22, and 23 are 2′-O-methyl nucleotides, nucleotides 2, 6, 14, and 16 are 2′-fluoro nucleotides, the first two internucleotide linkages at the 5′ terminus and the last two internucleotide linkages at the 3′ terminus are phosphorothioate internucleotide linkages, and each other internucleotide linkage is a phosphodiester internucleotide linkage; and wherein the sense strand is 21 nucleotides in length and the nucleotides of the sense strand are modified such that, counting from the 5′ terminus of the sense strand, nucleotides 1, 2, 3, 4, 5, 6, 8, 12, 13, 14, 15, 16, 17, 18, 19, 20, and 21 are 2′-O-methyl nucleotides, nucleotides 7, 9, 10, and 11 are 2′-fluoro nucleotides, the first two internucleotide linkages at the 5′ terminus and the last two internucleotide linkages at the 3′ terminus are phosphorothioate internucleotide linkages, and each other internucleotide linkage is a phosphodiester internucleotide linkage.
Embodiment 66. The compound of any one of embodiments 1 to 57, wherein the antisense strand is 23 nucleotides in length and the nucleotides of the antisense strand are modified such that, counting from the 5′ terminus of the antisense strand, nucleotides 1, 3, 4, 5, 7, 8, 9, 10, 11, 12, 13, 15, 17, 18, 19, 20, 21, 22, and 23 are 2′-O-methyl nucleotides, nucleotides 2, 6, 14, and 16 are 2′-fluoro nucleotides, the first two internucleotide linkages at the 5′ terminus and the last two internucleotide linkages at the 3′ terminus are phosphorothioate internucleotide linkages, and each other internucleotide linkage is a phosphodiester internucleotide linkage; and wherein the sense strand is 21 nucleotides in length and the nucleotides of the sense strand are modified such that, counting from the 5′ terminus of the sense strand, nucleotides 1, 2, 3, 4, 5, 6, 8, 12, 13, 14, 15, 16, 17, 18, 19, 20, and 21 are 2′-O-methyl nucleotides, nucleotides 7, 9, 10, and 11 are 2′-fluoro nucleotides, the first two internucleotide linkages at the 5′ terminus are phosphorothioate internucleotide linkages, and each other internucleotide linkage is a phosphodiester internucleotide linkage.
Embodiment 67. The compound of any one of embodiments 1 to 57, wherein the antisense strand is 23 nucleotides in length and wherein the nucleotides of the antisense strand are modified such that, counting from the 5′ terminus of the antisense strand, nucleotides 1, 3, 5, 7, 9, 11, 13, 15, 17, 19, 21, 22, and 23 are 2′-O-methyl nucleotides, nucleotides 2, 4, 6, 8, 10, 12, 14, 16, 18, and 20 are 2′-fluoro nucleotides, the first two internucleotide linkages at the 5′ terminus and the last two internucleotide linkages at the 3′ terminus are phosphorothioate internucleotide linkages, and each other internucleotide linkage is a phosphodiester internucleotide linkage; and wherein the sense strand is 21 nucleotides in length and wherein the nucleotides of the sense strand are modified such that, counting from the 5′ terminus of the sense strand, nucleotides 1 and 2 are 2′-O-methoxyethyl nucleotides, nucleotides 3, 4, 6, 8, 12, 14, 16, 18, 19, 20, and 21 are 2′-O-methyl nucleotides, nucleotides 5, 7, 9, 10, 11, 13, 15, and 17 are 2′-fluoro nucleotides, the first two internucleotide linkages at the 5′ terminus and the last two internucleotide linkages at the 3′ terminus are phosphorothioate internucleotide linkages, and each other internucleotide linkage is a phosphodiester internucleotide linkage.
Embodiment 68. The compound of any one of embodiments 1 to 57, wherein the antisense strand is 23 nucleotides in length and wherein the nucleotides of the antisense strand are modified such that, counting from the 5′ terminus of the antisense strand, nucleotides 1, 3, 5, 7, 9, 11, 13, 15, 17, 19, 21, 22, and 23 are 2′-O-methyl nucleotides, nucleotides 2, 4, 6, 8, 10, 12, 14, 16, 18, and 20 are 2′-fluoro nucleotides, the first two internucleotide linkages at the 5′ terminus and the last two internucleotide linkages at the 3′ terminus are phosphorothioate internucleotide linkages, and each other internucleotide linkage is a phosphodiester internucleotide linkage; and wherein the sense strand is 21 nucleotides in length and wherein the nucleotides of the sense strand are modified such that, counting from the 5′ terminus of the sense strand, nucleotides 2 and 3 are 2′-O-methoxyethyl nucleotides, nucleotides 1, 4, 6, 8, 12, 14, 16, 18, 19, 20, and 21 are 2′-O-methyl nucleotides, nucleotides 5, 7, 9, 10, 11, 13, 15, and 17 are 2′-fluoro nucleotides, the first two internucleotide linkages at the 5′ terminus and the last two internucleotide linkages at the 3′ terminus are phosphorothioate internucleotide linkages, and each other internucleotide linkage is a phosphodiester internucleotide linkage.
Embodiment 69. The compound of any one of embodiments 1 to 57, wherein the antisense strand is 23 nucleotides in length and wherein the nucleotides of the antisense strand are modified such that, counting from the 5′ terminus of the antisense strand, nucleotides 1, 3, 5, 7, 9, 11, 13, 15, 17, 19, 21, 22, and 23 are 2′-O-methyl nucleotides, nucleotides 2, 4, 6, 8, 10, 12, 14, 16, 18, and 20 are 2′-fluoro nucleotides, the first two internucleotide linkages at the 5′ terminus and the last two internucleotide linkages at the 3′ terminus are phosphorothioate internucleotide linkages, and each other internucleotide linkage is a phosphodiester internucleotide linkage; and wherein the sense strand is 21 nucleotides in length and wherein the nucleotides of the sense strand are modified such that, counting from the 5′ terminus of the sense strand, nucleotides 2, 3, 19 and 20 are 2′-O-methoxyethyl nucleotides, nucleotides 1, 4, 6, 8, 12, 14, 16, 18, and 21 are 2′-O-methyl nucleotides, nucleotides 5, 7, 9, 10, 11, 13, 15, and 17 are 2′-fluoro nucleotides, the first two internucleotide linkages at the 5′ terminus and the last two internucleotide linkages at the 3′ terminus are phosphorothioate internucleotide linkages, and each other internucleotide linkage is a phosphodiester internucleotide linkage.
Embodiment 70. The compound of any one of embodiments 1 to 57, wherein the antisense strand is 23 nucleotides in length and wherein the nucleotides of the antisense strand are modified such that, counting from the 5′ terminus of the antisense strand, nucleotides 1, 3, 5, 7, 9, 11, 13, 15, 17, 19, 21, 22, and 23 are 2′-O-methyl nucleotides, nucleotides 2, 4, 6, 8, 10, 12, 14, 16, 18, and 20 are 2′-fluoro nucleotides, the first two internucleotide linkages at the 5′ terminus and the last two internucleotide linkages at the 3′ terminus are phosphorothioate internucleotide linkages, and each other internucleotide linkage is a phosphodiester internucleotide linkage; and wherein the sense strand is 21 nucleotides in length and wherein the nucleotides of the sense strand are modified such that, counting from the 5′ terminus of the sense strand, nucleotides 1, 2, 3, and 4 are 2′-O-methoxyethyl nucleotides, nucleotides 6, 8, 12, 14, 16, 18, 19, 20 and 21 are 2′-O-methyl nucleotides, nucleotides 5, 7, 9, 10, 11, 13, 15, and 17 are 2′-fluoro nucleotides, the first two internucleotide linkages at the 5′ terminus and the last two internucleotide linkages at the 3′ terminus are phosphorothioate internucleotide linkages, and each other internucleotide linkage is a phosphodiester internucleotide linkage.
Embodiment 71. The compound of any one of embodiments 58 to 70, wherein the 5′ terminal phosphate group of the antisense strand is a 5′-(E)-vinylphosphonate group.
Embodiment 72. The compound of any one of embodiments 1 to 71, wherein the compound comprises a ligand covalently linked to one or more of the antisense strand and the sense strand of the double-stranded nucleic acid.
Embodiment 73. The compound of embodiment 72, wherein the ligand is squalene.
Embodiment 74. The compound of embodiment 72, wherein the compound has the structure:

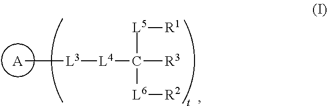

wherein A is the antisense strand and/or the sense strand of the double-stranded nucleic acid;
wherein t is an integer from 1 to 5;
L³ and L⁴ are independently a bond, —N(R²³)—, —O—, —S—, —C(O)—, —N(R²³)C(O)—, —C(O)N(R²⁴)—, —N(R²³)C(O)N(R²⁴)—, —C(O)O—, —OC(O)—, —N(R²³)C(O)O—, —OC(O)N(R²⁴)—, —OPO₂—O—, —O—P(O)(S)—O—, —O—P(O)(R²⁵)—O—, —O—P(S)(R²⁵)—O—, —O—P(O)(NR²³R²⁴)—N—, —O—P(S)(NR²³R²⁴)—N—, —O—P(O)(NR²³R²⁴)—O—, —O—P(S)(NR²³R²⁴)—O—, —P(O)(NR²³R²⁴)—N—, —P(S)(NR²³R²⁴)—N—, —P(O)(NR²³R²⁴)—O—, —P(S)(NR²³R²⁴)—O—, —S—S—, substituted or unsubstituted alkylene, substituted or unsubstituted heteroalkylene, substituted or unsubstituted cycloalkylene, substituted or unsubstituted heterocycloalkylene, substituted or unsubstituted arylene or substituted or unsubstituted heteroarylene;
L⁵ is -L^{5A}-L^{5B}-L^{5C}-L^{5D}-L^{5E}-;
L⁶ is -L^{6A}-L^{6B}-L^{6C}-L^{6D}-L^{6E}-;
R¹ and R² are independently unsubstituted C₁-C₂₅ alkyl, wherein at least one of R¹ and R² is unsubstituted C₉-C₁₉ alkyl;
R³ is hydrogen, —NH₂, —OH, —SH, —C(O)H, —C(O)NH₂, —NHC(O)H, —NHC(O)OH, —NHC(O)NH₂, —C(O)OH, —OC(O)H, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
L^{5A}, L^{5B}, L^{5C}, L^{5D}, L^{5E}, L^{6A}, L^{6B}, L^{6C}, L^{6D}, and L^{6E} are independently a bond, —NH—, —O—, —S—, —C(O)—, —NHC(O)—, —NHC(O)NH—, —C(O)O—, —OC(O)—, —C(O)NH—, substituted or unsubstituted alkylene, substituted or unsubstituted heteroalkylene, substituted or unsubstituted cycloalkylene, substituted or unsubstituted heterocycloalkylene, substituted or unsubstituted arylene or substituted or unsubstituted heteroarylene; and
each R²³, R²⁴ and R²⁵ is independently hydrogen or unsubstituted C₁-C₁₀ alkyl.
Embodiment 75. The compound of embodiment 74, wherein t is 1.
Embodiment 76. The compound of embodiment 74, wherein t is 2.
Embodiment 77. The compound of embodiment 74, wherein t is 3.
Embodiment 78. The compound of any one of embodiments 74 to 77, wherein A is the sense strand.
Embodiment 79. The compound of any one of embodiments 74 to 78, wherein A is the antisense strand.
Embodiment 80. The compound of one of embodiments 74 to 79, wherein each of R²³, R²⁴ and R²⁵ is independently hydrogen or unsubstituted C₁-C₃ alkyl.
Embodiment 81. The compound of one of embodiments 74 to 80, wherein one L³ is attached to a 3′ carbon of a nucleotide.
Embodiment 82. The compound of embodiment 81, wherein the 3′ carbon is the 3′ carbon of a 3′ terminal nucleotide.
Embodiment 83 The compound of one of embodiments 74 to 78, wherein one L³ is attached to a 5′ carbon of a nucleotide.
Embodiment 84. The compound of embodiment 83, wherein the 5′ carbon is the 5′ carbon of a 5′ terminal nucleotide.
Embodiment 85. The compound of one of embodiments 74 to 78, wherein one L³ is attached to a 2′ carbon of a nucleotide.
Embodiment 86. The compound of one of embodiments 74 to 85, wherein L³ and L⁴ are independently a bond, —NH—, —O—, —C(O)—, —C(O)O—, —OC(O)—, —OPO₂—O—, —O—P(O)(S)—O—, —O—P(O)(CH₃)—O—, —O—P(S)(CH₃)—O—, —O—P(O)(N(CH₃)₂)—N—, —O—P(O)(N(CH₃)₂)—O—, —O—P(S)(N(CH₃)₂)—N—, —O—P(S)(N(CH₃)₂)—O—, —P(O)(N(CH₃)₂)—N—, —P(O)(N(CH₃)₂)—O—, —P(S)(N(CH₃)₂)—N—, —P(S)(N(CH₃)₂)—O—, substituted or unsubstituted alkylene or substituted or unsubstituted heteroalkylene.
Embodiment 87. The compound of one of embodiments 74 to 86, wherein L³ is independently

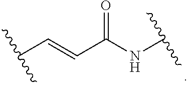 [SMILES: */C=C/C(=O)N*]

Embodiment 88. The compound of one of embodiments 74 to 86, wherein L³ is independently —OPO₂—O— or —OP(O)(S)—O—.
Embodiment 89. The compound of one of embodiments 74 to 86, wherein L³ is independently —O—.
Embodiment 90. The compound of any one of embodiments 74 to 86, wherein L³ is independently —C(O)—.
Embodiment 91. The compound of any one of embodiments 74 to 86, wherein L³ is independently —O—P(O)(N(CH₃)₂)—N—.
Embodiment 92. The compound of one of embodiments 74 to 89, wherein L⁴ is independently substituted or unsubstituted alkylene or substituted or unsubstituted heteroalkylene.
Embodiment 93. The compound of one of embodiments 74 to 92, wherein L⁴ is independently -L⁷-NH—C(O)— or -L⁷-C(O)—NH—, wherein L⁷ is substituted or unsubstituted alkylene.
Embodiment 94. The compound of one of embodiments 74 to 93, wherein L⁴ is independently

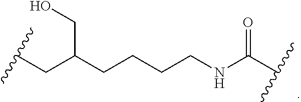 [SMILES: *CC(CO)CCCCNC(*)=O]

Embodiment 95. The compound of one of embodiments 74 to 93, wherein L⁴ is independently

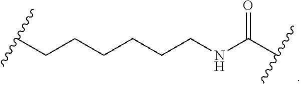 [SMILES: *CCCCCCNC(*)=O]

Embodiment 96. The compound of one of embodiments 74 to 95, wherein -L³-L⁴- is independently —O-L⁷-NH—C(O)— or —O-L⁷-C(O)—NH—, wherein L⁷ is independently substituted or unsubstituted alkylene, substituted or unsubstituted heteroalkylene, or substituted or unsubstituted heteroalkenylene.
Embodiment 97. The compound of embodiment 96, wherein -L³-L⁴- is independently —O-L⁷-NH—C(O)—, wherein L⁷ is independently substituted or unsubstituted C₅-C₈ alkylene.
Embodiment 98. The compound of embodiment 97, wherein -L³-L⁴- is independently

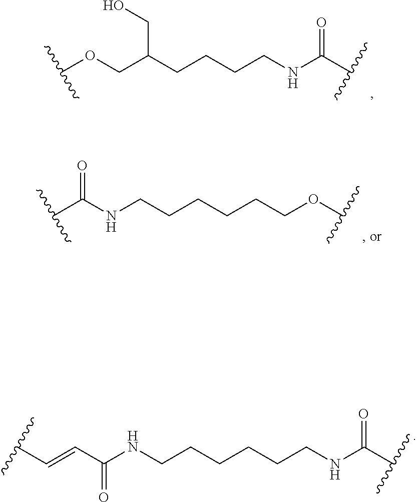 [SMILES: *OCC(CO)CCCCNC(*)=O ; *OCCCCCCNC(*)=O ; */C=C/C(=O)NCCCCCCNC(*)=O]

Embodiment 99. The compound of one of embodiments 74 to 86, wherein -L³-L⁴- is independently —OPO₂—O-L⁷-NH—C(O)—, —OP(O)(S)—O-L⁷-NH—C(O)—, —OPO₂—O-L⁷-C(O)—NH— or —OP(O)(S)—O-L⁷-C(O)—NH—, wherein L⁷ is independently substituted or unsubstituted alkylene.
Embodiment 100. The compound of embodiment 99, wherein -L³-L⁴- is independently —OPO₂—O-L⁷-NH—C(O)— or —OP(O)(S)—O-L⁷-NH—C(O)—, wherein L⁷ is independently substituted or unsubstituted C₅-C₈ alkylene.
Embodiment 101. The compound of embodiment 100, wherein -L³-L⁴- is independently

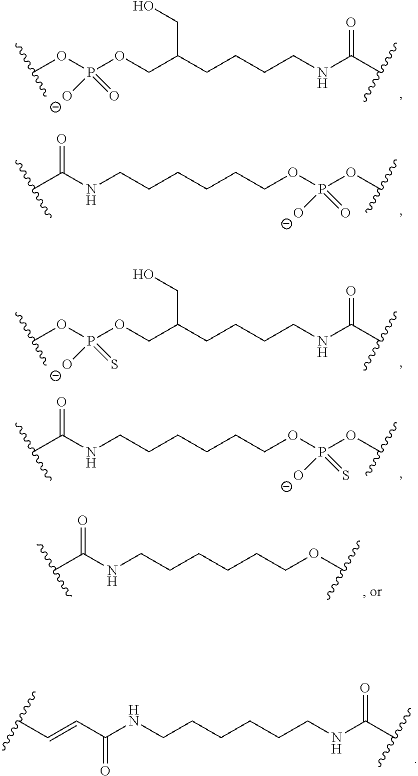 [SMILES: *OP(=O)([O-])OCC(CO)CCCCNC(*)=O ; *OP(=O)([O-])OCCCCCCNC(*)=O ; *OP([O-])(=S)OCC(CO)CCCCNC(*)=O ; *OP([O-])(=S)OCCCCCCNC(*)=O ; *OCCCCCCNC(*)=O ; */C=C/C(=O)NCCCCCCNC(*)=O]

Embodiment 102. The compound of embodiment 101, wherein an -L³-L⁴- is independently

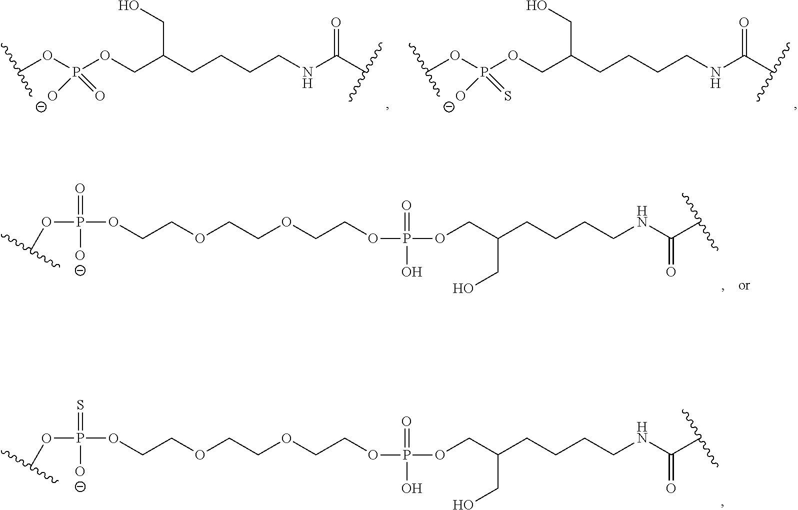 [SMILES: *OP(=O)([O-])OCC(CO)CCCCNC(*)=O ; *OP([O-])(=S)OCC(CO)CCCCNC(*)=O ; *OP(=O)([O-])OCCOCCOCCOP(=O)(O)OCC(CO)CCCCNC(*)=O ; *OP([O-])(=S)OCCOCCOCCOP(=O)(O)OCC(CO)CCCCNC(*)=O]

and is attached to the 3′ carbon of a 3′ terminal nucleotide.
Embodiment 103. The compound of embodiment 101, wherein an -L³-L⁴- is independently

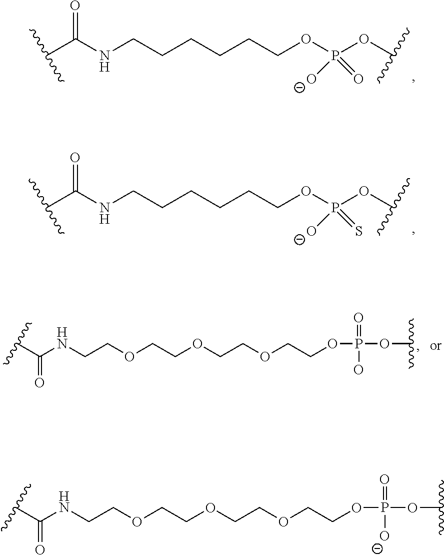 [SMILES: *OP(=O)([O-])OCCCCCCNC(*)=O ; *OP([O-])(=S)OCCCCCCNC(*)=O ; *OP(=O)(O)OCCOCCOCCOCCNC(*)=O ; *OP(=O)([O-])OCCOCCOCCOCCNC(*)=O]

and is attached to the 5′ carbon of a 5′ terminal nucleotide.
Embodiment 104. The compound of embodiment 101 wherein an -L³-L⁴- is independently

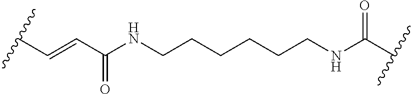 [SMILES: */C=C/C(=O)NCCCCCCNC(*)=O]

and is attached to a 2′ carbon.
Embodiment 105. The compound of one of embodiments 71 to 104, wherein R³ is independently hydrogen.
Embodiment 106. The compound of one of embodiments 71 to 105, wherein L⁶ is independently —NHC(O)—, —C(O)NH—, substituted or unsubstituted alkylene, or substituted or unsubstituted heteroalkylene.
Embodiment 107. The compound of embodiment 106, wherein L⁶ is independently —NHC(O)—.
Embodiment 108. The compound of embodiment 106, wherein
   L^{6A} is independently a bond or unsubstituted alkylene;
   L^{6B} is independently a bond, —NHC(O)—, or unsubstituted arylene;
   L^{6C} is independently a bond, unsubstituted alkylene, or unsubstituted arylene;
   L^{6D} is independently a bond or unsubstituted alkylene; and
   L^{6E} is independently a bond or —NHC(O)—.
   Embodiment 109. The compound of embodiment 106, wherein
   L^{6A} is independently a bond or unsubstituted C₁-C₈ alkylene;
   L^{6B} is independently a bond, —NHC(O)—, or unsubstituted phenylene;
   L^{6C} is independently a bond, unsubstituted C₂-C₈ alkynylene, or unsubstituted phenylene;
   L^{6D} is independently a bond or unsubstituted C₁-C₈ alkylene; and
   L^{6E} is independently a bond or —NHC(O)—.
Embodiment 110. The compound of one of embodiments 71 to 105, wherein L⁶ is independently a bond,

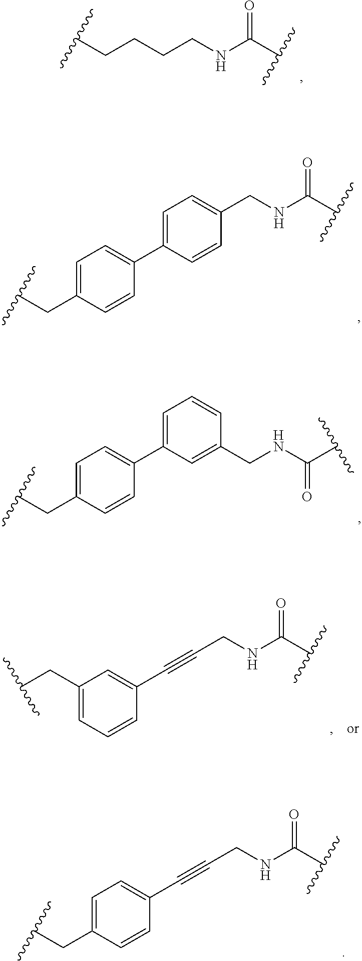 [SMILES: *CCCCNC(*)=O ; *Cc1ccc(-c2ccc(CNC(*)=O)cc2)cc1 ; *Cc1ccc(-c2cccc(CNC(*)=O)c2)cc1 ; *Cc1cccc(C#CCNC(*)=O)c1 ; *Cc1ccc(C#CCNC(*)=O)cc1]

Embodiment 111. The compound of one of embodiments 71 to 110, wherein L⁵ is independently —NHC(O)—, —C(O)NH—, substituted or unsubstituted alkylene, or substituted or unsubstituted heteroalkylene.
Embodiment 112. The compound of one of embodiments 71 to 110, wherein L⁵ is independently —NHC(O)—.
Embodiment 113. The compound of one of embodiments 71 to 110, wherein
   L^{5A} is independently a bond or unsubstituted alkylene;
   L^{5B} is independently a bond, —NHC(O)—, or unsubstituted arylene;
   L^{5C} is independently a bond, unsubstituted alkylene, or unsubstituted arylene;
   L^{5D} is independently a bond or unsubstituted alkylene; and
   L^{5E} is independently a bond or —NHC(O)—.
   Embodiment 114. The compound of one of embodiments 71 to 110, wherein
   L^{5A} is independently a bond or unsubstituted C₁-C₈ alkylene;
   L^{5B} is independently a bond, —NHC(O)—, or unsubstituted phenylene;
   L^{5C} is independently a bond, unsubstituted C₂-C₈ alkynylene, or unsubstituted phenylene;
   L^{5D} is independently a bond or unsubstituted C₁-C₈ alkylene; and
   L^{5E} is independently a bond or —NHC(O)—.
Embodiment 115. The compound of one of embodiments 71 to 110, wherein L⁵ is independently a bond,

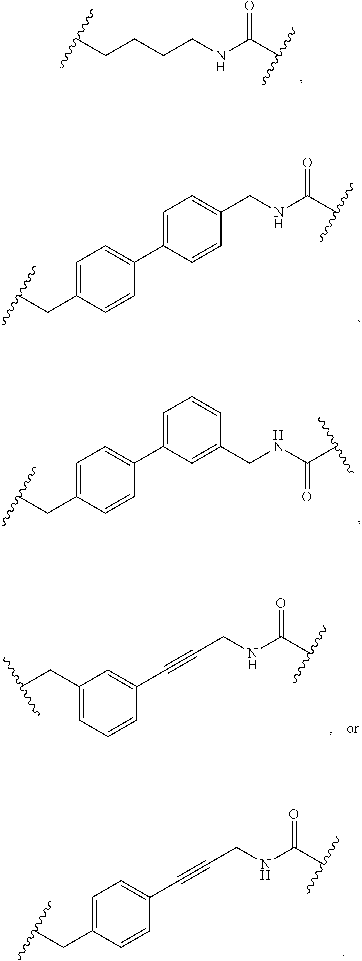 [SMILES: *CCCCNC(*)=O ; *Cc1ccc(-c2ccc(CNC(*)=O)cc2)cc1 ; *Cc1ccc(-c2cccc(CNC(*)=O)c2)cc1 ; *Cc1cccc(C#CCNC(*)=O)c1 ; *Cc1ccc(C#CCNC(*)=O)cc1]

Embodiment 116. The compound of one of embodiments 71 to 110, wherein R¹ is unsubstituted C₁-C₁₇ alkyl.
Embodiment 117. The compound of one of embodiments 71 to 110, wherein R¹ is unsubstituted C₁₁-C₁₇ alkyl.
Embodiment 118. The compound of one of embodiments 71 to 110, wherein R¹ is unsubstituted C₁₃-C₁₇ alkyl.
Embodiment 119. The compound of one of embodiments 71 to 110, wherein R¹ is unsubstituted C₁₄-C₁₅ alkyl.
Embodiment 120. The compound of one of embodiments 71 to 110, wherein R¹ is unsubstituted unbranched C₁-C₁₇ alkyl.
Embodiment 121. The compound of one of embodiments 71 to 110, wherein R¹ is unsubstituted unbranched C₁₁-C₁₇ alkyl.
Embodiment 122. The compound of one of embodiments 71 to 110, wherein R¹ is unsubstituted unbranched C₁₃-C₁₇ alkyl.
Embodiment 123. The compound of one of embodiments 71 to 110, wherein R¹ is unsubstituted unbranched C₁₄-C₁₅ alkyl.
Embodiment 124. The compound of one of embodiments 71 to 110, wherein R¹ is unsubstituted unbranched saturated C₁-C₁₇ alkyl.
Embodiment 125. The compound of one of embodiments 71 to 110, wherein R¹ is unsubstituted unbranched saturated C₁₁-C₁₇ alkyl.
Embodiment 126. The compound of one of embodiments 71 to 110, wherein R¹ is unsubstituted unbranched saturated C₁₃-C₁₇ alkyl.
Embodiment 127. The compound of one of embodiments 71 to 110, wherein R¹ is unsubstituted unbranched saturated C₁₄-C₁₅ alkyl.
Embodiment 128. The compound of one of embodiments 71 to 127, wherein R² is unsubstituted C₁-C₁₇ alkyl.
Embodiment 129. The compound of one of embodiments 71 to 127, wherein R² is unsubstituted C₁₁-C₁₇ alkyl.
Embodiment 130. The compound of one of embodiments 71 to 127, wherein R² is unsubstituted C₁₃-C₁₇ alkyl.
Embodiment 131. The compound of one of embodiments 71 to 127, wherein R² is unsubstituted C₁₄-C₁₅ alkyl.
Embodiment 132. The compound of one of embodiments 71 to 127, wherein R² is unsubstituted unbranched C₁-C₁₇ alkyl.
Embodiment 133. The compound of one of embodiments 71 to 127, wherein R² is unsubstituted unbranched C₁₁-C₁₇ alkyl.
Embodiment 134. The compound of one of embodiments 71 to 127, wherein R² is unsubstituted unbranched C₁₃-C₁₇ alkyl.
Embodiment 135. The compound of one of embodiments 71 to 127, wherein R² is unsubstituted unbranched C₁₄-C₁₅ alkyl.
Embodiment 136. The compound of one of embodiments 71 to 127, wherein R² is unsubstituted unbranched saturated C₁-C₁₇ alkyl.
Embodiment 137. The compound of one of embodiments 71 to 127, wherein R² is unsubstituted unbranched saturated C₁₁-C₁₇ alkyl.
Embodiment 138. The compound of one of embodiments 71 to 127, wherein R² is unsubstituted unbranched saturated C₁₃-C₁₇ alkyl.
Embodiment 139. The compound of one of embodiments 71 to 127, wherein R² is unsubstituted unbranched saturated C₁₄-C₁₅ alkyl.
Embodiment 140. The compound of any one of embodiments 71 to 139, wherein the ligand is covalently linked to the antisense strand.
Embodiment 141. The compound of any one of embodiments 71 to 139, wherein the ligand is covalently linked to the sense strand.
Embodiment 142. The compound of embodiment 74, wherein -L³-L⁴-
   is

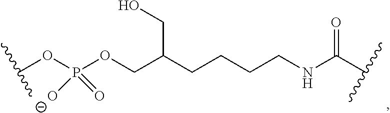 [SMILES: *OP(=O)([O-])OCC(CO)CCCCNC(*)=O]

the phosphate group of -L³-L⁴- is attached to the 3′ carbon of the 3′ terminal nucleotide of the sense strand,
   L⁶ is

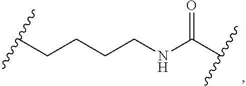 [SMILES: *CCCCNC(*)=O]

L⁵ is —NHC(O)—,
R³ is hydrogen,
R¹ is unsubstituted unbranched C₁₅ alkyl, and
R² is unsubstituted unbranched C₁₅ alkyl.
Embodiment 143. The compound of embodiment 74, wherein -L³-L⁴- is

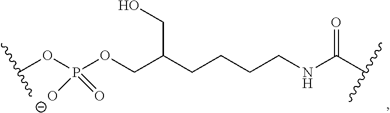 [SMILES: *OP(=O)([O-])OCC(CO)CCCCNC(*)=O]

the phosphate group of -L³-L⁴- to the 3′ carbon of the 3′ terminal nucleotide of the sense strand,
   L⁶ is

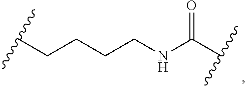 [SMILES: *CCCCNC(*)=O]

L⁵ is —NHC(O)—,
R³ is hydrogen,
R¹ is unsubstituted unbranched C₁₃ alkyl, and
R² is unsubstituted unbranched C₁₃ alkyl.
Embodiment 144. The compound of embodiment 74, wherein the compound is selected from any one of DT-000544, DT-000545, DT-000546, DT-000620, DT-000621, DT-000622, DT-000623, DT-000624, DT-000625, DT-000626, DT-000627, DT-000628, DT-000811, DT-000812, DT-000945, DT-000959, DT-000960, DT-000961, DT-000962, DT-000963, DT-000964, DT-000965, DT-000966, DT-000967, DT-001037, DT-001038, DT-001039, DT-001044, DT-001045, DT-001046, DT-001047, DT-001048, DT-001049, DT-001050, DT-001051, DT-001052, DT-001053, DT-001054, DT-001055, DT-001056, DT-001057, DT-001058, DT-001059, DT-001060, DT-001061, DT-001109, DT-001110, DT-001111, DT-001112, DT-001113, DT-001114, DT-001115, DT-001116, DT-001117, DT-001118, DT-001119, DT-001120, DT-001121, DT-001122, DT-001123, DT-001124, DT-001125, DT-001126, DT-001127, DT-001128, DT-001129, DT-001130, DT-001131, DT-001132, DT-001145, DT-001146, DT-001147, DT-001148, DT-001149, DT-001150, DT-001151, DT-001152, DT-001153, DT-001154, DT-001155, DT-001156, DT-001157, DT-001158, DT-001159, DT-001160, DT-001161, DT-001162, DT-001163, DT-001164, DT-001176, DT-001177, DT-001178, DT-001179, DT-001180, DT-001181, DT-001182, DT-001183, DT-001184, DT-001185, DT-001186, DT-001187, DT-001188, DT-001189, DT-001190, DT-001191, DT-001192, DT-001193, DT-001194, DT-001195, DT-001196, DT-001197, DT-001198, DT-001199, DT-001200, DT-001201, DT-001202, DT-001203, DT-001204, DT-001205, DT-001206, DT-001207, DT-001208, DT-001217, DT-001218, DT-001219, DT-001220, DT-001221, DT-001222, DT-001223, DT-001224, DT-001230, DT-001231, DT-001232, DT-001233, DT-001234, DT-001235, DT-001236, DT-001237, DT-001238, DT-001239, DT-001240, DT-001241, DT-001242, DT-001243, DT-001246, DT-001247, DT-001248, DT-001249, DT-001250, DT-001251, DT-001252, DT-001253, DT-001254, DT-001255, DT-001256, DT-001257, DT-001261, DT-001262, DT-001263, DT-001264, DT-001265, DT-001266, DT-001267, DT-001276, DT-001277, DT-001278, DT-001279, DT-001280, DT-001281, DT-001282, DT-001283, DT-001296, DT-001297, DT-001298, DT-001299, DT-001300, DT-001301, DT-001302, DT-001303, DT-001304, DT-001305, DT-001306, DT-001307, DT-001322, DT-001323, DT-001324, DT-001325, DT-001326, DT-001327, DT-001328, DT-001329, DT-001330, DT-001331, DT-001332, DT-001333, DT-001334, DT-001335, DT-001344, DT-001345, DT-001346, DT-001347, DT-001348, DT-001349, DT-001350, DT-001351, DT-001355, DT-001356, DT-001357, DT-001358, DT-001359, DT-001360, DT-001361, DT-001362, DT-001363, DT-001364, DT-001365, DT-001366, DT-001367, DT-001368, and DT-001369.
Embodiment 145. The compound of embodiment 74, wherein the compound is DT-000623.
Embodiment 146. The compound of embodiment 74, wherein the compound is DT-000812.
Embodiment 147. The compound of embodiment 74, wherein the compound is DT-001246.
Embodiment 148. The compound of embodiment 74, wherein the compound is DT-001247.
Embodiment 149. The compound of embodiment 74, wherein the compound is DT-001250.
Embodiment 150. The compound of embodiment 74, wherein the compound is DT-001251.
Embodiment 151. The compound of embodiment 74, wherein the compound is DT-001252.
Embodiment 152. The compound of embodiment 74, wherein the compound is DT-001253.
Embodiment 153. The compound of embodiment 74, wherein the compound is DT-001254.
Embodiment 154. The compound of embodiment 74, wherein the compound is DT-001255.
Embodiment 155. The compound of embodiment 74, wherein the compound is DT-001256.
Embodiment 156. The compound of embodiment 74, wherein the compound is DT-001257.
Embodiment 157. The compound of any one of embodiments 1 to 156, wherein the compound is present as a pharmaceutical salt.
Embodiment 158. The compound of embodiment 157, wherein the salt is a sodium salt.
Embodiment 159. The compound of any one of embodiments 1 to 158, wherein the compound is present in a pharmaceutically acceptable diluent.
Embodiment 160. The compound of embodiment 159, wherein the pharmaceutically acceptable diluent is a sterile aqueous solution.
Embodiment 161. The compound of embodiment 160, wherein the sterile aqueous solution is a sterile saline solution.
Embodiment 162. A pharmaceutical composition comprising the compound of any one of embodiments 1 to 161.
Embodiment 163. A method of inhibiting the expression of peripheral myelin protein 22 (PMP22) mRNA in a cell, comprising contacting the cell with a compound of any one of embodiments 1 to 161, thereby inhibiting the expression of PMP22 mRNA in the cell.
Embodiment 164. The method of embodiment 163, wherein the cell is a peripheral nerve cell.
Embodiment 165. The method of embodiment 164, wherein the cell is in vitro.
Embodiment 166. The method of embodiment 164, wherein the cell is in vivo.
Embodiment 167. A method of inhibiting the expression of peripheral myelin protein 22 (PMP22) mRNA in a subject, comprising administering to the subject an effective amount of a compound of any one of embodiments 1 to 161 or the pharmaceutical composition of embodiment 162, thereby inhibiting the expression of peripheral myelin protein 22 (PMP22) mRNA.
Embodiment 168. The method of embodiment 167, wherein the expression of PMP22 mRNA is inhibited in a peripheral nerve of the subject.
Embodiment 169. The method of embodiment 168, wherein the peripheral nerve is one or more of a sciatic nerve, a brachial plexus nerve, a tibial nerve, a peroneal nerve, a femoral nerve, a lateral femoral cutaneous nerve, and a spinal accessory nerve.
Embodiment 170. A method for increasing myelination and/or slowing the loss of myelination in a subject, comprising administering to the subject an effective amount of a compound of any one of embodiments 1 to 161 or the pharmaceutical composition of embodiment 162.
Embodiment 171. The method of embodiment 170, wherein the administering increases myelination in the subject.
Embodiment 172. The method of embodiment 170 or 171, wherein the administering slows the loss of myelination in the subject.
Embodiment 173. The method of any one of embodiments 167 to 172, wherein the subject has a peripheral demyelinating disease.
Embodiment 174. The method of embodiment 173, wherein the administration of the compound treats the peripheral demyelinating disease.
Embodiment 175. The method of embodiment 173 or 174, wherein the peripheral demyelinating disease is Charcot-Marie-Tooth disease (CMT).
Embodiment 176. The method of embodiment 175, wherein the CMT is Charcot-Marie-Tooth disease Type 1A (CMT1A).
Embodiment 177. A method of treating Charcot-Marie-Tooth disease (CMT), comprising administering to a subject in need thereof an effective amount of a compound of any one of embodiments 1 to 161 or the pharmaceutical composition of embodiment 162.
Embodiment 178. The method of embodiment 177, wherein the Charcot-Marie-Tooth disease is Charcot-Marie-Tooth disease Type 1A (CMT1A).
Embodiment 179. The method of embodiment 178, wherein the subject is diagnosed as having CMT1A by the presence of one or more of: a family history of CMT1A; amplification of the PMP22 gene; distal muscle weakness; distal musculature atrophy; reduced deep tendon reflexes, distal sensory impairment; reduced compound muscle action potential; and reduced nerve conduction velocity.
Embodiment 180. The method of any one of embodiments 167 to 179, wherein the administration improves or slows the progression of one or more clinical indicators of CMT1A in the subject, wherein the one or more clinical indicators is selected from: distal muscle weakness;
distal musculature atrophy;
reduced deep tendon reflexes;
distal sensory impairment;
reduced nerve conduction velocity;
reduced compound muscle action potential;
reduced sensory nerve action potential;
increased calf muscle fat fraction;
elevated plasma neurofilament light (NfL); and/or elevated plasma transmembrane protease serine 5 (TMPRSS55).
Embodiment 181. The method of embodiment 179 or 180, wherein the distal muscle weakness is reduced hand grip strength and/or reduced foot dorsiflexion.
Embodiment 182. The method of any one of embodiments 179 to 181, wherein the distal muscle weakness is measured by quantified muscular testing (QMT).
Embodiment 183. The method of embodiment 179 or 180, wherein the nerve conduction velocity is selected from motor nerve conduction velocity and sensory nerve conduction velocity.
Embodiment 184. The method of embodiment 183, wherein the nerve conduction velocity is measured by electroneurography.
Embodiment 185. The method of embodiment 179 or 180, wherein compound muscle action potential is measured by electromyogram.
Embodiment 186. The method of embodiment 179 or 180, wherein the distal musculature atrophy is calf muscle atrophy.
Embodiment 187. The method of embodiment 186, wherein calf muscle fat fraction is measured by magnetic resonance imaging.
Embodiment 188. The method of any one of embodiments 179 to 187, wherein disease severity and/or disease progression in a subject is determined by one or more clinical assessments, wherein the clinical assessment is selected from Charcot-Marie-Tooth Neuropathy Score (CMTNS), Charcot-Marie-Tooth Neuropathy Score with Rasch weighting (CMTNS-R), Charcot Marie-Tooth Neuropathy Score Version 2 (CMTNS-v2), Charcot-Marie-Tooth Examination Score (CMTES), Charcot-Marie-Tooth Examination Score with Rasch weighting (CMTES-R), Charcot-Marie-Tooth Functional Outcome Measure (CMT-FOM), Charcot-Marie-Tooth Disease Pediatric Scale, Charcot-Marie-Tooth Disease Infant Scale, Charcot-Marie-Tooth Health Index, and Overall Neuropathy Limitation Scale (ONLS).
Embodiment 189. The method of embodiment 188, wherein disease progression in the subject comprises measuring the change over time in the one or more clinical assessments.
Embodiment 190. The method of any one of embodiments 167 to 189, wherein the administration is intravenous administration or subcutaneous administration.
Embodiment 191. The method of any one of embodiments 167 to 190, comprising administering at least one additional therapy to the subject.
Embodiment 192. Use of the compound of any one of embodiments 1 to 161 in therapy.
Embodiment 193. Use of the compound of any one of embodiments 1 to 161 for the treatment of Charcot-Marie-Tooth disease Type 1A (CMT1A).
Embodiment 194. Use of the pharmaceutical composition of embodiment 162 for the treatment of Charcot-Marie-Tooth disease Type 1A (CMT1A).

### Examples

The following examples are presented to more fully illustrate some embodiments of the invention. They should not be construed, however, as limiting the scope of the invention. Variations of these examples within the scope of the claims are within the purview of one skilled in the art and are considered to fall within the scope of the embodiments as described and claimed herein. The reader will recognize that the skilled artisan, armed with the present disclosure and skill in the art, is able to prepare and use the invention without exhaustive examples.

### Example 1: Synthesis of Uptake Motifs and Conjugation of Uptake Motifs to Oligonucleotides

### Synthesis of Uptake Motif DTx-01-08

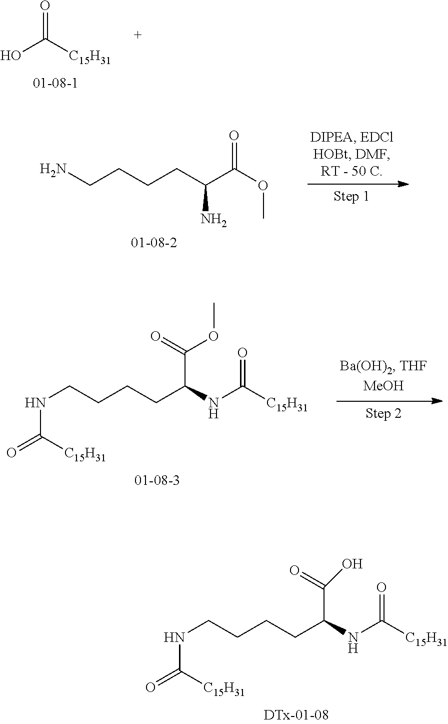

### Step 1: Synthesis of Compound 01-08-3

To a stirred solution of linear fatty acid 01-08-1 (25.58 g, 0.099 mol) in DMF (500 mL) at RT was added DIPEA (42.66 mL, 0.245 mol) and compound 01-08-2 (8.0 g, 0.049 mol), followed by EDCl (18.97 g, 0.099 mol) and HOBt (13.37 g, 0.099 mol). The resulting mixture was stirred at 50° C. After 16 h, the reaction mixture was quenched with ice water and extracted with DCM. The combined organic extract was washed with water, brine, dried over Na₂SO₄, and then evaporated to give crude 01-08-3, which was recrystallized (20% MTBE in petroleum ether) to afford 01-08-3 as an off-white solid (18 g, 56%).

### Step 2: Synthesis of Lipid Motif DTx-01-08

To a stirred solution of 01-08-3 (10 g, 0.0156 mol) in MeOH and THF (1:1; 200 mL) at RT was added slowly Ba(OH)₂ (9.92 g, 0.031 mol, dissolved in MeOH). The resulting mixture was stirred at RT. After 6 h, the reaction mixture was quenched with ice water dropwise, and then acidified with 1.5 M HCl. The mixture was filtered, and the precipitate was recrystallized (MTBE in petroleum ether) to afford lipid motif DTx-01-08 as an off-white solid (7.2 g, 74.2%). MS (ESI) m/z (M+H)⁺: 623.6; ¹H-NMR (400 MHz, CDCl₃): δ 0.868 (m, 6H), 1.25-1.69 (m, 58H), 2.03 (t, J=7.2 Hz, 2H), 2.11 (t, J=7.6 Hz, 2H), 2.99 (q, J=8.4 Hz, 2H), 4.15-4.20 (m, 1H), 7.42 (br s, 1H), 7.65 (d, J=7.6 Hz, 1H), 12.09 (br s, 1H).

### Synthesis of Lipid Motif DTx-01-32

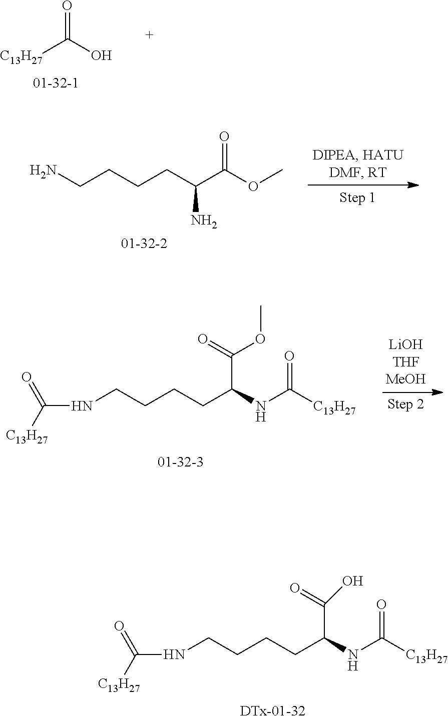

### Step 1: Synthesis of Intermediate 01-32-3

To a stirred solution of 01-32-2 (3 g, 0.01 mol) in DMF (50 mL) at RT was added slowly DIPEA (13.8 mL, 0.077 mol), linear fatty acid 01-32-1 (4.4 g, 0.0154 mol), and HATU (5.87 g, 0.0154 mol). The resulting mixture was stirred at 60° C. After 16 h, the reaction mixture was quenched with ice water, the solids isolated by filtration, and the solids dried under vacuum to afford 01-32-3 as an off-white solid (3.5 g, 53.2%).

### Step 2: Synthesis of Lipid Motif DTx-01-32

To a stirred solution of 01-32-3 (3.5 g, 0.0051 mol) in MeOH (10 mL), THF (10 mL), and water (3 mL), was added LiOH H₂O (0.8 g, 0.0154). The reaction mixture was stirred 16 h. Subsequently, the reaction mixture was concentrated under vacuum and neutralized with 1.5 N HCl. The solids were isolated by filtration, washed with water, and dried under vacuum, affording crude DTx-01-32. Recrystallization (80% DCM in hexane) yielded lipid motif DTx-01-32 as an off-white solid (2.3 g, 79.3%). LCMS m/z (M+H)+: 567.2; ¹H-NMR (400 MHz, TFA-d): δ 0.87-0.98 (m, 6H), 1.20-1.58 (m, 41H), 1.74-1.92 (m, 8H), 2.18-2.21 (m, 2H), 2.73 (t, J=7.6 Hz, 2H), 3.05 (t, J=7.6 Hz, 2H), 3.60 (t, J=7.8 Hz, 2H).

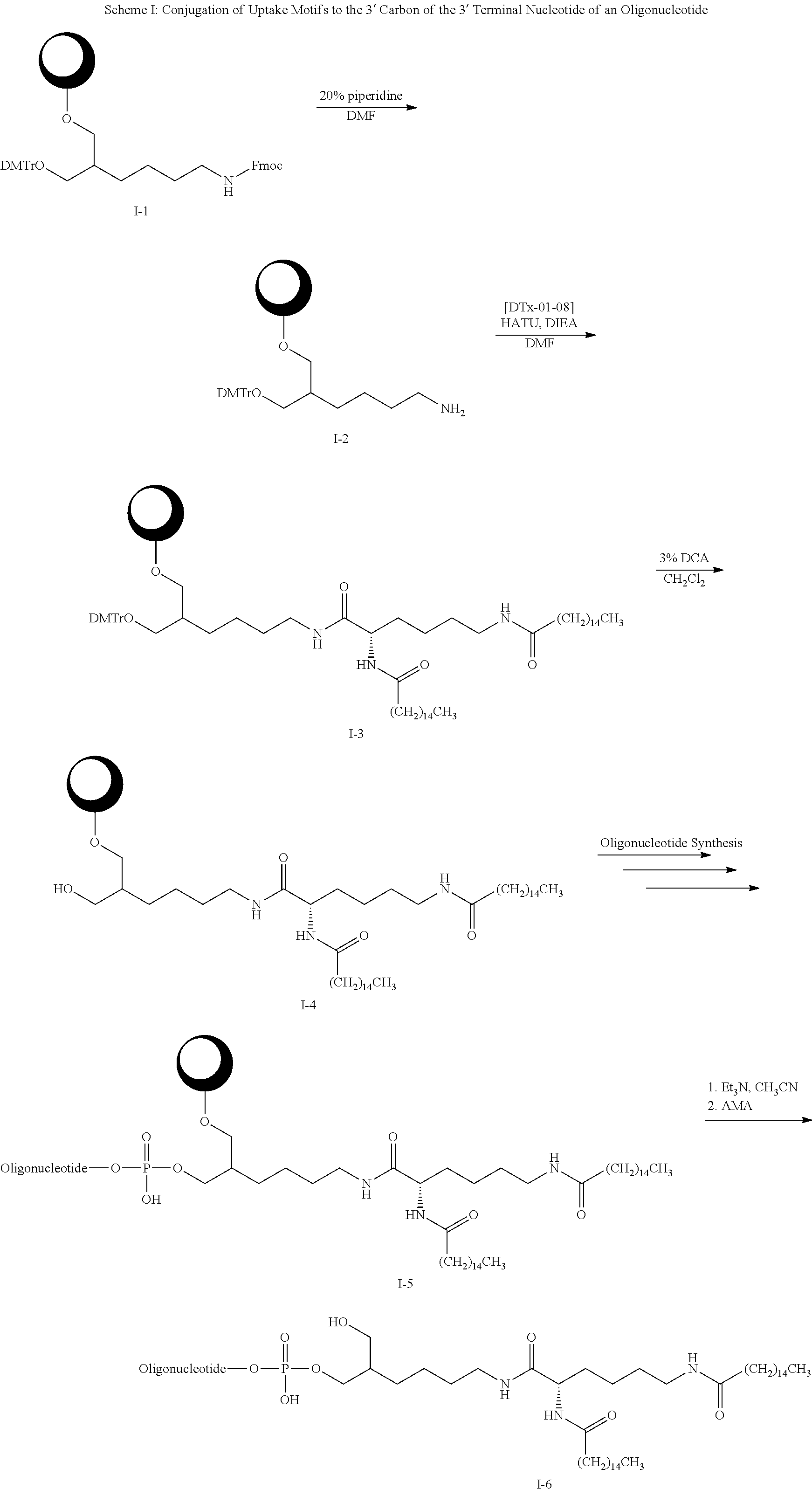

Scheme I above illustrates the preparation of an oligonucleotide conjugated with an uptake motif at the 3′ terminus of the oligonucleotide, i.e. at the 3′ carbon of the terminal 3′ nucleotide. In summary, 3′-amino CPG beads I-1 (Glen Research, Catalog No. 20-2958) modified with the DMT and Fmoc-protected C7 linker illustrated above were treated with 20% piperidine/DMF to afford Fmoc-deprotected amino C7 CPG beads I-2. An uptake motif (e.g. DTx-01-08) was then coupled to I-2 using HATU and DIEA in DMF to produce lipid-loaded CPG beads I-3, which were treated by 3% dichloroacetic acid (DCA) in DCM to remove the DMT protecting group and afford I-4. Oligonucleotide synthesis was accomplished via standard phosphoramidite chemistry and yielded oligonucleotide-bounded CPG beads I-5. At this point, if applicable, beads I-5 containing methyl ester-protected lipid motifs (e.g., DTx-01-07-OMe, DTx-01-09-OMe) were saponified to their respective carboxylic acid using 0.5 M LiOH in 3:1 v/v methanol/water. Subsequent treatment of I-5 with AMA [ammonium hydroxide (28%)/methylamine (40%) (1:1, v/v)] cleaved the DTx-01-08-conjugated oligonucleotide from the beads. The conjugated oligonucleotide was then purified by RP-HPLC and characterized by MALDI-TOF MS using the [M+H] peak.

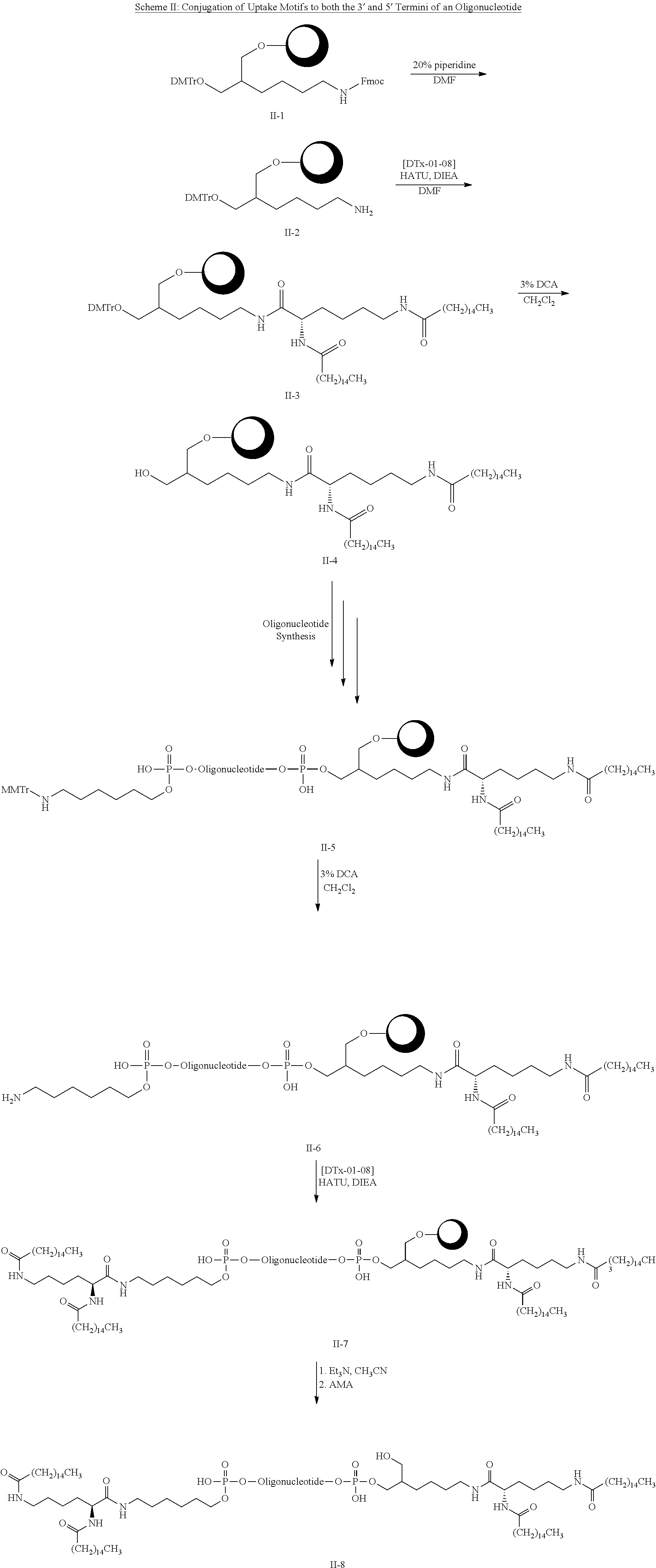

Scheme II above illustrates the preparation of a sense strand of a double-stranded oligonucleotide conjugated with an uptake motif at each of the 5′ and 3′ termini. In summary, 3′-amino CPG beads 11-1 (Glen Research, Catalog No. 20-2958) modified with the DMT and Fmoc-protected C7 linker illustrated above were treated with 20% piperidine/DMF to afford Fmoc-deprotected amino C7 CPG beads II-2. An uptake motif (e.g. DTx-01-08) was then coupled to 11-2 using HATU and DIEA in DMF to produce the fatty-acid loaded CPG beads II-3, which were subsequently treated with 3% dichloroacetic acid (DCA) in DCM to remove the DMT protecting group and afford 11-4. Oligonucleotide synthesis was performed on 11-4 via standard phosphoramidite chemistry. The final coupling was with a phosphoramidite (Glen Research, Catalog No. 10-1906) that incorporated a monomethoxytrityl (MMTr) protected 6-carbon alkyl amine as shown in structure 11-5. After removal of MMT with 3% dichloroacetic acid (DCA) in DCM, II-6 was coupled to DTx-01-08 using HATU and DIEA in DMF to yield 11-7. Stepwise deprotection with triethylamine in acetonitrile (to remove phosphate protecting groups) and AMA [ammonium hydroxide (28%)/methylamine (40%) (1:1, v/v)](to remove base protecting groups and cleave the oligonucleotide from the synthesis resin) yielded crude II-8. Purification using RP-HPLC yielded a conjugated oligonucleotide. Purity and identity of 11-8 were confirmed by analytical RP-HPLC and MALDI-TOF MS using the [M+H] peak, respectively.

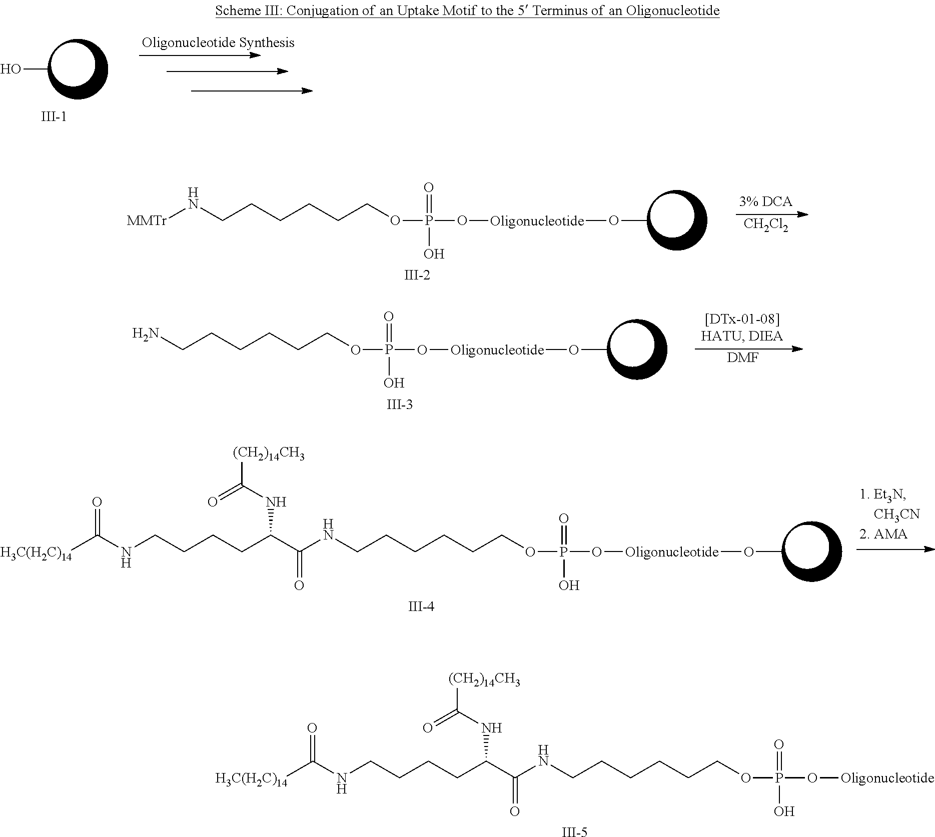

Scheme III above illustrates the preparation of an oligonucleotide conjugated to an uptake motif at the 5′ terminus, i.e. at the 5′ carbon of the 3′ terminal nucleotide. In summary, oligonucleotide synthesis was performed on CPG beads III-1 (Glen Research, Catalog No. 20-5041-xx) via standard phosphoramidite chemistry. In the last nucleotide coupling of the automated sequence, a nucleotide modified with the MMT-protected C₆ linker illustrated above (Glen Research, Catalog No. 10-1906) was used, yielding modified oligonucleotide-bounded CPG beads III-2. After removal of MMT with 3% dichloroacetic acid (DCA) in DCM, 111-2 was coupled to an uptake motif (e.g., DTx-01-08) using HATU and DIEA in DMF to yield III-4. Subsequent treatment with AMA [ammonium hydroxide (28%)/methylamine (40%) (1:1, v/v)] cleaved the DTx-01-08-conjugated modified oligonucleotide from the beads to generate III-5. The oligonucleotide was then purified by RP-HPLC and characterized by MALDI-TOF MS using the [M+H] peak.

### Duplex Formation

For each of the strands synthesized by Schemes I, II, or III and listed above, the corresponding complementary strand was prepared via standard phosphoramidite chemistry, purified by IE-HPLC, and characterized by MALDI-TOF MS using the [M+H] peak. The duplex was formed by mixing equal molar equivalents of the passenger strand (the sense strand) and guide strand (the antisense strand), heating to 90° C. for 5 minutes, and then slowly cooling to room temperature. Duplex formation was confirmed by non-denaturing PAGE or non-denaturing HPLC.

### Example 2: Biology Experimental Methods

Cell Culture. HEK293 cells were purchased from ATCC and were cultured in DMEM containing 10% Fetal Bovine Serum (FBS), 2 mM L-glutamine, 1× non-essential amino acids, 100 U/mL penicillin and 100 mg/mL streptomycin in a humidified 37° C. incubator with 5% CO₂. Human Schwann cells (HSwC), isolated from human spinal nerve and cryopreserved at first passage (P1), were purchased from iXcells Biotechnologies (Cat #10HU-188). HSwC were cultured in Schwann Cell Growth Medium (Cat #MD-0055) in a humidified 37° C. incubator with 5% CO₂.

Generation of Stable Human and Mouse PMP22 Cell Lines. 3×10^{\hat{ }6} HEK293 cells were plated onto 10-cm tissue culture treated petri dishes in the media described herein without antibiotics. The day after plating, human (Origene, Cat #RC216500) or mouse (Origene, Cat #MR225485) PMP22 plasmids were transfected into HEK293 cells with Lipofectamine 2000 according to the manufacturer's protocol. Briefly, 20 μg of each plasmid were diluted in 480 μL of DMEM without FBS or antibiotic. Separately, 50 μL of Lipofectamine 2000 was diluted in 450 μL of DMEM without FBS or antibiotic. The plasmid/DMEM and the Lipofectamine 2000/DMEM cocktails were then combined, mixed by titrating up and down and incubated for 20 minutes at room temperature to enable complex formation. The DMEM media containing FBS but lacking antibiotic (9 mL) was then added to the plasmid/Lipofectamine 2000 complexes (1 mL) and then added to cells in the 10-cm dish. The cells were incubated overnight at 37° C. in the incubator. Media was then removed and replaced with DMEM containing FBS and antibiotic. Five days post-transfection, the media was replaced with DMEM containing FBS, antibiotic and 800 μg/mL geneticin to select for cells that stably express either the human or mouse PMP22. The cells were cultured in this media for 30 days with media changes every 3 days. The cells were then expanded and subsequently cryopreserved. Sequencing and qPCR were utilized to confirm integration of the human or mouse PMP22 expression vector.

Reverse Transfection of siRNA. HEK293 cells were trypsinized and diluted to 20,000 cells/well, in 90 μL of antibiotic-free media. Schwann cells were trypsinized and diluted to 10,000 cells/well, in 90 μL of antibiotic-free media. Compounds were diluted in PBS to 100× of the desired final concentration. Separately, Lipofectamine RNAiMax (Life Technologies) was diluted 1:66.7 in media lacking supplements (e.g. FBS, antibiotic etc.). The 100× compound in PBS was then complexed with RNAiMAX by adding 1 part compound in PBS to 9 parts lipofectamine/media. Following incubation for 20 minutes, 10 μL of the compound: RNAiMAX complexes were added to a 96-well collagen coated plate. A volume of 90 ul of the cell dilution was added to each well of the 96-well plate. The plate was then placed in a humidified 37° C. incubator with 5% CO₂. After 24 hours, the complexes were removed and replaced with complete media containing antibiotics for each cell line. HEK293 media was replaced with DMEM containing 10% FBS, 2 mM L-glutamine, 1× non-essential amino acids, 100 U/mL penicillin and 100 mg/mL streptomycin. Schwann cell media was replaced with Schwann Cell Growth Medium. RNA was isolated 48 hours following transfection.

Free uptake of conjugated siRNA. HEK293 cells were trypsinized and diluted to 20,000 cells/well, in 100 μL of complete media and allowed to settle overnight in 96 well collagen coated plates. Schwann cells were trypsinized and diluted to 10,000 cells/well, in 100 μL of complete media and allowed to settle for 48 hours in 96 well collagen coated plates. Compounds were diluted in deep well plates in the corresponding basal media for each cell line supplemented with 2% FBS to the desired final concentration of the top dose then serially diluted. After the appropriate amount of time for cells to settle, media was removed from plates by inverting. 100 ul of compound or PBS at proper concentrations was added to each well of the 96 well plate. HEK293 cells were incubated for 48 hours, and Schwann cells were incubated 72 hours in a humidified 37° C. incubator with 5% CO₂ before RNA was isolated.

RNA Isolation, Reverse Transcription and Quantitative PCR. RNA was isolated utilizing the RNeasy 96 kit (Qiagen) according to the manufacturer's protocol. RNA was reverse transcribed to cDNA utilizing random primers and the high-capacity cDNA reverse transcription kit (ThermoFisher Scientific) in a SimpliAmp thermal cycler (ThermoFisher Scientific) according to the manufacturer's instructions. Real-time quantitative PCR was performed utilizing gene-specific primers (Thermofisher Scientific; IDTDNA), TaqMan probes (Thermofisher Scientific; IDTDNA) and TaqMan fast universal PCR master mix (Thermofisher scientific) on a StepOnePlus real-time PCR system (Thermofisher Scientific) according to the manufacturer's instructions. For analysis of quantitative PCR, mRNA expression was normalized to the expression of either 18s rRNA, b-actin or HPRT1 mRNA (housekeeping genes) utilizing the relative CT method according to the best practices proposed in Nature Protocols (Schmittgen, T. D. & Livak, K. J. Analyzing real-time PCR data by the comparative C(T) method. Nat Protoc 3, 1101-1108 (2008)).

Mice. C3-PMP22 (B6.Cg-Tg(PMP22)C₃Fbas/J) male mice were originally purchased from the Jackson Laboratory. C3-PMP22 mice express 3 to 4 copies of a wild-type human peripheral myelin protein 22 (PMP22). The C3-PMP22 male mice were used to set up a mouse colony. The transgenic line was maintained hemizygous by breeding C3-PMP22 males with wildtype females (C₅₇BL/6J). All litters were weaned between 21-23 days of age and tail clipped for genotyping. Both hemizygous female and male mice were used for experiments.

Intravenous injection. Mice were weighed the day before the study initiation. On the day of the study, the mice were restrained with an approved device and injected with the treatment of interest (compound or PBS) via the tail vein.

Target Engagement Studies in vivo in wildtype mice and C3-PMP22 mice. 7-84 days following intravenous injection of the compound of interest or control, the mice were euthanized. Sciatic, tibial, sensory, and motor branches of the femoral nerves and/or brachial plexus were dissected and prepared for RNA isolation. The regions of interest were placed in tubes containing beads, flash frozen and stored at −80° C. until RNA isolation. To extract total RNA, Trizol was added to the tubes and RNA isolated using the RNeasy 96 kit via the manufacturer's instructions.

Electrophysiology assessment using Electromyography (EMG). The EMG apparatus (ADInstruments, PowerLab Cat #PL2604/P) was used to measure motor nerve conduction velocity (MNCV). The mice were anesthetized in an isoflurane chamber and transferred to the nose cone on a recirculating water heating pad to maintain their temperature. A rectal probe was used to monitor the temperature. A total of 4 electrodes were used: 2 recording and 2 stimulating electrodes. The two recording electrodes were gently inserted between the 1st and 2nd and 2nd and 3rd toes and taped to the plexiglass surface. One stimulating electrode was inserted under the skin between the shoulders. The second stimulating electrode was inserted into the skin of the ankle. The EMG was set to deliver a stimulus using a 0.1 msec square pulse stimulus every 2 seconds. The stimulation voltage was gradually increased until the maximal M-wave is observed (Mmax). The stimulating electrode was then moved from the ankle to the greater sciatic notch and stimulate once. The stimulation was repeated at the ankle and sciatic notch 2 more times each. At the end of the last measurement, leaving the electrode at the hip, the electrodes from the toes were removed and the leg stretched. A compass was used to measure the distance between the electrode at the hip and the point at the ankle at which stimulation was conducted. The latency between the M-wave in response to stimulation at the ankle vs hip was calculated and averaged across the 3 trials. This value was divided by the distance between the electrodes to calculate the motor conduction velocity. At the end of the measurement all electrodes were removed, and the mouse was placed on a water-recirculating heating pad that is set at 37° C. Once the mouse has fully recovered it was returned to housing rack in animal holding room.

Myelin staining. The nerves of interest were carefully dissected, placed lengthwise on a stick of wood (applicator or matchstick) to prevent the nerve from folding, and immersed in a scintillation vial containing cold 2.5% glutaraldehyde (fixative) overnight at 4° C. The following day the nerves were washed with 0.1M sodium phosphate buffer and immersed in 2% osmium for approximately 1 hour (osmium penetrates tissue from all sides at roughly 0.5 mm/hr, so a mouse nerve with a diameter of 1 mm should osmicate for 1 hour). After rinsing in water, the nerves were dehydrated and embedded in resin blocks. Once embedded in resin blocks the nerves were cut with glass knifes using a microtome in 0.15 um sections. The sections were subsequently stained with 2% paraphenylenediamine (PPD) for 20 minutes at room temperature, rinsed, dried and coverslip mounted for microscopic examination.

Beam Walking. Coordination and balance were evaluated through the beam walking assay by two experimenters that were blinded to experimental conditions. Mice were trained over two-three consecutive days to cross a 100 cm-long painted wood round beam with a 25 mm diameter to reach a platform with a darkened escape box. The beam was place 30 cm over a padded surface. Training trials ended when the mouse reached the escape platform or when the mouse fell off the beam. The latency to cross the beam and the number of times the hind paws slipped during placement were tabulated for each training run. Each training run was repeated three times per day with a minimum of 5 minutes between runs. Training was considered complete when all mice crossed the beam consistently without pausing. On the subsequent testing day, mice underwent three trials in which they crossed the 25 mm-diameter beam, with a minimum of 5 minutes between runs. Then mice underwent an additional three trials in which they crossed a 10 mm-diameter beam. Latency to cross the beam and the number of foot slips or falls were tabulated for each trial. Data from the second and third trials on each beam were averaged. Trials in which the mouse paused while crossing or fell off the beam were excluded from analysis.

Hindlimb clasping. In order to evaluate general neuromuscular dysfunction, incidence of hindlimb clasping was observed. A blinded observer took a photo of hindlimb behavior while suspending the mice briefly from their tails. From these images, hindlimb behavior was scored as 0-normal splaying of the hindlimbs and toes of the paw spread wide, 1-clasping of one foot or hindlimb, or 2-clasping of both feet of hindlimb. The angle of hindlimb spread was also calculated from the images using ImageJ2 (NIH, Rueden et al, 2017) to measure the angle between the hind paws by drawing a vector from each paw to the anus.

Grip strength. Grip strength is a measure of muscular strength, or the maximum force/tension generated by one's forearm muscles. It can be measured using a digital force meter equipped with precision force gauges to retain the peak force applied on a digital display and with a grid or wire system that allows mouse grip by either or both paws. Each mouse was lifted by the tail to the height where the front paws are at the same height as the bar/grid. The mouse was then moved horizontally towards the bar/grid until it was within reach. After visually checking that the grip was good, i.e. a symmetric, tight grip with both paws and exerting a detectable resistance against the investigator's pull, the mouse was gently pulled away until its grasp is broken. The pulling was at a constant speed and sufficiently slow to permit the mouse to build up a resistance against it. The transducer saved the value at this point. Measurements were discarded if the animal used only one paw or also used its hind paws, turned backwards during the pull, or released the bar without resistance. The test was repeated three times and the values averaged.

### Example 3: Unconjugated siRNAs Targeting PMP22

Numerous siRNAs targeting the human PMP22 mRNA were designed and synthesized. The sense and antisense strands of the compounds ere prepared with sugar moiety, terminal, and internucleotide linkage modifications to increase hybridization affinity, minimize degradation by nucleases, and enhance loading into RISC. The siRNAs are shown in Table 3.

In Table 3, “Start” and “End” correspond to the 5′ and 3′ nucleotide positions of the nucleotide sequence of the human PMP22 mRNA (NCBI Reference Sequence N_{M}_000304.4, deposited with GenBank on Nov. 22, 2018; SEQ ID NO: 1170) to which the nucleotides of the antisense strand are complementary. Each row represents a sense and antisense strand pair of an siRNA. If present, an siRNA ID in the “Parent siRNA ID” column indicates an siRNA related by nucleotide sequence.

Modified sugar moieties are indicated by a subscript notation following the nucleotide, and modified internucleotide linkages are indicated by a superscript notation. A nucleotide followed by the subscript “F” is a 2′-fluoro nucleotide; a nucleotide followed by the subscript “M” is a 2′-O-methyl nucleotide; and a nucleotide followed by the subscript “D” is a beta-D-deoxyribonucleotide. A superscript “S” is a phosphorothioate internucleotide linkage; all other internucleotide linkages are phosphodiester internucleotide linkages. For example, “U_{F}^{S}C_{M}” is a 2′-fluorouridine linked to a 2′-O-methylcytidine by a phosphorothioate internucleotide linkage. “G_{M}U_{F}” is a 2-O-methylguanosine linked to a 2′-fluorouridine by a phosphodiester internucleotide linkage. A hydroxyl group is at the 5′ carbon of the 5′ terminal nucleotide is indicated by “5′-OH”; a phosphate group at the 5′ carbon of the 5′ terminal nucleotide is indicated by “5′-PO4”; and a hydroxyl group at the 3′ carbon of the 3′ terminal nucleotide is indicated by “OH-3′.”

TABLE 3
Unconjugated siRNAs targeting PMP22

| | | | | | | Unmodi- | | | | | Unmodi- | |
|---|---|---|---|---|---|---|---|---|---|---|---|---|
| | | | | Modified | | fied | | | Modified | | fied | |
| | | | | Nucleo- | SEQ | Nucleo- | SEQ | | Nucleo- | SEQ | Nucleo- | SEQ |
| siRNA | | | Strand | tide | ID | tide | ID | Strand | tide | ID | tide | ID |
| ID | Start | End | ID | Sequence | NO | Sequence | NO | ID | Sequence | NO | Sequence | NO |
| DT- | 211 | 229 | DTS- | 5′-OH- | 1 | CUCCUCC | 352 | DTS- | 5′-PO4- | 152 | UACUCA | 592 |
| 000390 | | | 000568 | C_{F}^{S}U_{M}^{S}C_{F}C_{M}U | | UGUUGC | | 000569 | U_{M}^{S}A_{F}^{S}C_{M}U_{F}C | | GCAACA | |
| | | | | C_{M}C_{F}U_{M}G_{F}U | | UGAGUA | | | _{M}A_{F}G_{M}C_{F}A_{M}A | | GGAGGA | |
| | | | | _{M}U_{F}G_{M}C_{F}U_{M}G | | TT | | | C_{M}A_{F}G_{M}G_{F}A | | GTT | |
| | | | | _{F}A_{M}G_{F}U_{M}A_{F}^{S}T | | | | | _{M}G_{F}G_{M}A_{F}G_{M}^{S} | | | |
| | | | | _{D}^{S}T_{D}-OH-3′ | | | | | T_{D}^{S}T_{D}-OH-3′ | | | |
| DT- | 285 | 303 | DTS- | 5′-OH- | 2 | CCAAUG | 348 | DTS- | 5′-PO4- | 153 | AUUGCCC | 571 |
| 000391 | | | 000570 | C_{F}^{S}C_{M}^{S}A_{F}A_{M}U | | GAUCGU | | 000571 | A_{M}^{S}U_{F}^{S}U_{M}G_{F}C | | ACGAUCC | |
| | | | | _{F}G_{M}G_{F}A_{M}U_{F}C | | GGGCAA | | | _{M}C_{F}C_{M}A_{F}C_{M}G_{F} | | AUUGGTT | |
| | | | | _{M}G_{F}U_{M}G_{F}G_{M}G | | UTT | | | A_{M}U_{F}C_{M}C_{F}A_{M} | | | |
| | | | | _{F}C_{M}A_{F}A_{M}U_{F}^{S}T | | | | | U_{F}U_{M}G_{F}G_{M}^{S}T_{D} | | | |
| | | | | _{D}^{S}T_{D}-OH-3′ | | | | | ^{S}T_{D}-OH-3′ | | | |
| DT- | 311 | 329 | DTS- | 5′-OH- | 3 | CAACUG | 344 | DTS- | 5′-PO4- | 154 | UUCUGCC | 617 |
| 000392 | | | 000572 | C_{F}^{S}A_{M}^{S}A_{F}C_{M}U | | AUCUCU | | 000573 | U_{M}^{S}U_{F}^{S}C_{M}U_{F}G | | AGAGAU | |
| | | | | _{F}G_{M}A_{F}U_{M}C_{F}U | | GGCAGA | | | _{M}C_{F}C_{M}A_{F}G_{M}A | | CAGUUGT | |
| | | | | _{M}C_{F}U_{M}G_{F}G_{M}C | | ATT | | | _{F}G_{M}A_{F}U_{M}C_{F}A | | T | |
| | | | | _{F}A_{M}G_{F}A_{M}A_{F}^{S}T | | | | | _{M}G_{F}U_{M}U_{F}G_{M}^{S} | | | |
| | | | | _{D}^{S}T_{D}-OH-3′ | | | | | T_{D}^{S}T_{D}-OH-3′ | | | |
| DT- | 336 | 354 | DTS- | 5′-OH- | 4 | CACCUCU | 345 | DTS- | 5′-PO4- | 155 | AUUUCC | 573 |
| 000393 | | | 000574 | C_{F}^{S}A_{M}^{S}C_{F}C_{M}U | | UCCUCAG | | 000575 | A_{M}^{S}U_{F}^{S}U_{M}U_{F}C | | UGAGGA | |
| | | | | _{F}C_{M}U_{F}U_{M}C_{F}C_{M} | | GAAAUTT | | | _{M}C_{F}U_{M}G_{F}A_{M}G | | AGAGGU | |
| | | | | U_{F}C_{M}A_{F}G_{M}G_{F} | | | | | _{F}G_{M}A_{F}A_{M}G_{F}A | | GTT | |
| | | | | A_{M}A_{F}A_{M}U_{F}^{S}T_{D} | | | | | _{M}G_{F}G_{M}U_{F}G_{M}^{S} | | | |
| | | | | ^{S}T_{D}-OH-3′ | | | | | T_{D}^{S}T_{D}-OH-3′ | | | |
| DT- | 349 | 367 | DTS- | 5′-OH- | 5 | GGAAAU | 358 | DTS- | 5′-PO4- | 156 | AACAGU | 500 |
| 000394 | | | 000576 | G_{F}^{S}G_{M}^{S}A_{F}A_{M}A | | GUCCACC | | 000577 | A_{M}^{S}A_{F}^{S}C_{M}A_{F}G | | GGUGGA | |
| | | | | _{F}U_{M}G_{F}U_{M}C_{F}C | | ACUGUUT | | | _{M}U_{F}G_{M}G_{F}U_{M}G | | CAUUUCC | |
| | | | | _{M}A_{F}C_{M}C_{F}A_{M}C_{F} | | T | | | _{F}G_{M}A_{F}C_{M}A_{F}U | | TT | |
| | | | | U_{M}G_{F}U_{M}U_{F}^{S}T_{D} | | | | | _{M}U_{F}U_{M}C_{F}C_{M}^{S}T | | | |
| | | | | ^{S}T_{D}-OH-3′ | | | | | _{D}^{S}T_{D}-OH-3′ | | | |
| DT- | 365 | 383 | DTS- | 5′-OH- | 6 | GUUUCU | 362 | DTS- | 5′-PO4- | 157 | UUUGGU | 640 |
| 000395 | | | 000578 | G_{F}^{S}U_{M}^{S}U_{F}U_{M}C | | CAUCAUC | | 000579 | U_{M}^{S}U_{F}^{S}U_{M}G_{F}G | | GAUGAU | |
| | | | | _{F}U_{M}C_{F}A_{M}U_{F}C | | ACCAAAT | | | _{M}U_{F}G_{M}A_{F}U_{M}G | | GAGAAA | |
| | | | | _{M}A_{F}U_{M}C_{F}A_{M}C | | T | | | _{F}A_{M}U_{F}G_{M}A_{F}G | | CTT | |
| | | | | C_{M}A_{F}A_{M}A_{F}^{S}T | | | | | _{M}A_{F}A_{M}A_{F}C_{M}^{S}T | | | |
| | | | | _{D}^{S}T_{D}-OH-3′ | | | | | _{D}^{S}T_{D}-OH-3′ | | | |
| DT- | 420 | 438 | DTS- | 5′-OH- | 7 | GUCGAU | 360 | DTS- | 5′-PO4- | 158 | AAUGCU | 514 |
| 000396 | | | 000580 | G_{F}^{S}U_{M}C_{F}G_{M}A | | CAUCUUC | | 000581 | A_{M}^{S}A_{F}^{S}U_{M}G_{F}C | | GAAGAU | |
| | | | | _{F}U_{M}C_{F}A_{M}U_{F}C | | AGCAUUT | | | _{M}U_{F}G_{M}A_{F}A_{M}G | | GAUCGA | |
| | | | | _{M}U_{F}U_{M}C_{F}A_{M}G | | T | | | _{F}A_{M}U_{F}G_{M}A_{F}U | | CTT | |
| | | | | C_{M}A_{F}U_{M}U_{F}^{S}T | | | | | _{M}C_{F}G_{M}A_{F}C_{M}^{S}T | | | |
| | | | | _{D}^{S}T_{D}-OH-3′ | | | | | _{D}^{S}T_{D}-OH-3′ | | | |
| DT- | 447 | 465 | DTS- | 5′-OH- | 8 | GUUCCU | 361 | DTS- | 5′-PO4- | 159 | UUGGCA | 627 |
| 000397 | | | 000582 | G_{F}^{S}U_{M}^{S}U_{F}C_{M}C | | GUUCUU | | 000583 | U_{M}^{S}U_{F}^{S}G_{M}G_{F}C | | GAAGAA | |
| | | | | _{F}U_{M}G_{F}U_{M}U_{F}C | | CUGCCAA | | | _{M}A_{F}G_{M}A_{F}A_{M}G | | CAGGAA | |
| | | | | _{M}U_{F}U_{M}C_{F}U_{M}G | | TT | | | _{F}A_{M}A_{F}C_{M}A_{F}G | | CTT | |
| | | | | C_{M}C_{F}A_{M}A_{F}^{S}T | | | | | _{M}G_{F}A_{M}A_{F}C_{M}^{S}T | | | |
| | | | | _{D}^{S}T_{D}-OH-3′ | | | | | _{D}^{S}T_{D}-OH-3′ | | | |
| DT- | 499 | 517 | DTS- | 5′-OH- | 9 | AUCACU | 343 | DTS- | 5′-PO4- | 160 | UUUGGA | 639 |
| 000398 | | | 000584 | A_{F}^{S}U_{M}^{S}C_{F}A_{M}C | | GGAAUC | | 000585 | U_{M}^{S}U_{F}^{S}U_{M}G_{F}G | | AGAUUC | |
| | | | | _{F}U_{M}G_{F}G_{M}A_{F}A | | UUCCAA | | | _{M}A_{F}A_{M}G_{F}A_{M}U | | CAGUGA | |
| | | | | _{M}U_{F}C_{M}U_{F}U_{M}C | | ATT | | | _{F}U_{M}C_{F}C_{M}A_{F}G | | UTT | |
| | | | | C_{M}A_{F}A_{M}A_{F}^{S}T | | | | | _{M}U_{F}G_{M}A_{F}U_{M}^{S} | | | |
| | | | | _{D}^{S}T_{D}-OH-3′ | | | | | T_{D}^{S}T_{D}-OH-3′ | | | |
| DT- | 504 | 522 | DTS- | 5′-OH- | 10 | UGGAAU | 372 | DTS- | 5′-PO4- | 161 | AAGAAU | 501 |
| 000399 | | | 000586 | U_{F}^{S}G_{M}^{S}G_{F}A_{M}A | | CUUCCAA | | 000587 | A_{M}^{S}A_{F}^{S}G_{M}A_{F}A | | UUGGAA | |
| | | | | _{F}U_{M}C_{F}U_{M}U_{F}C | | AUUCUUT | | | _{M}U_{F}U_{M}U_{F}G_{M}G | | GAUUCC | |
| | | | | _{M}C_{F}A_{M}A_{F}A_{M}U | | T | | | _{F}A_{M}A_{F}G_{M}A_{F}U | | ATT | |
| | | | | _{F}U_{M}C_{F}U_{M}U_{F}^{S}T | | | | | _{M}U_{F}C_{M}C_{F}A_{M}^{S}T | | | |
| | | | | _{D}^{S}T_{D}-OH-3′ | | | | | _{D}^{S}T_{D}-OH-3′ | | | |
| DT- | 578 | 596 | DTS- | 5′-OH- | 11 | GGCAUC | 359 | DTS- | 5′-PO4- | 162 | UAAUCC | 589 |
| 000400 | | | 000588 | G_{F}^{S}G_{M}^{S}C_{F}A_{M}U | | UCAACUC | | 000589 | U_{M}^{S}A_{F}^{S}A_{M}U_{F}C | | GAGUUG | |
| | | | | _{F}C_{M}U_{F}C_{M}A_{F}A | | GGAUUA | | | _{M}C_{F}G_{M}A_{F}G_{M}U | | AGAUGC | |
| | | | | _{M}C_{F}U_{M}C_{F}G_{M}G | | TT | | | _{F}U_{M}G_{F}A_{M}G_{F}A | | CTT | |
| | | | | _{F}A_{M}U_{F}U_{M}A_{F}^{S}T | | | | | _{M}U_{F}G_{M}C_{F}C_{M}^{S}T | | | |
| | | | | _{D}^{S}T_{D}-OH-3′ | | | | | _{D}^{S}T_{D}-OH-3′ | | | |
| DT- | 596 | 614 | DTS- | 5′-OH- | 12 | ACUCCUA | 340 | DTS- | 5′-PO4- | 163 | UAGGCG | 596 |
| 000401 | | | 000590 | A_{F}^{S}C_{M}^{S}U_{F}C_{M}C | | CGGUUU | | 000591 | U_{M}^{S}A_{F}^{S}G_{M}G_{F}C | | AAACCG | |
| | | | | _{F}U_{M}A_{F}C_{M}G_{F}G | | CGCCUAT | | | _{M}G_{F}A_{M}A_{F}A_{M}C | | UAGGAG | |
| | | | | _{M}U_{F}U_{M}U_{F}C_{M}G | | T | | | _{F}C_{M}G_{F}U_{M}A_{F}G | | UTT | |
| | | | | _{F}C_{M}C_{F}U_{M}A_{F}^{S}T | | | | | _{M}G_{F}A_{M}G_{F}U_{M}^{S} | | | |
| | | | | _{D}^{S}T_{D}-OH-3′ | | | | | T_{D}^{S}T_{D}-OH-3′ | | | |
| DT- | 313 | 331 | DTS- | 5′-OH- | 13 | ACUGAU | 341 | DTS- | 5′-PO4- | 164 | AGUUCU | 537 |
| 000402 | | | 000592 | A_{F}^{S}C_{M}^{S}U_{F}G_{M}A | | CUCUGGC | | 000593 | A_{M}^{S}G_{F}^{S}U_{M}U_{F}C | | GCCAGA | |
| | | | | _{F}U_{M}C_{F}U_{M}C_{F}U | | AGAACUT | | | _{M}U_{F}G_{M}C_{F}C_{M}A | | GAUCAG | |
| | | | | _{M}G_{F}G_{M}C_{F}A_{M}G | | T | | | _{F}G_{M}A_{F}G_{M}A_{F}U | | UTT | |
| | | | | _{F}A_{M}A_{F}C_{M}U_{F}^{S}T | | | | | _{M}C_{F}A_{M}G_{F}U_{M}^{S}T | | | |
| | | | | _{D}^{S}T_{D}-OH-3′ | | | | | _{D}^{S}T_{D}-OH-3′ | | | |
| DT- | 314 | 332 | DTS- | 5′-OH- | 14 | CUGAUC | 354 | DTS- | 5′-PO4- | 165 | CAGUUC | 577 |
| 000403 | | | 000594 | C_{F}^{S}U_{M}^{S}G_{F}A_{M}U | | UCUGGC | | 000595 | C_{M}^{S}A_{F}^{S}G_{M}U_{F}U | | UGCCAG | |
| | | | | _{F}C_{M}U_{F}C_{M}U_{F}G | | AGAACU | | | _{M}C_{F}U_{M}G_{F}C_{M}C_{F} | | AGAUCA | |
| | | | | _{M}G_{F}C_{M}A_{F}G_{M}A | | GTT | | | A_{M}G_{F}A_{M}G_{F}A_{M} | | GTT | |
| | | | | _{F}A_{M}C_{F}U_{M}GE^{S}T | | | | | U_{F}C_{M}A_{F}G_{M}T_{D} | | | |
| | | | | _{D}^{S}T_{D}-OH-3′ | | | | | ^{S}T_{D}-OH-3′ | | | |
| DT- | 315 | 333 | DTS- | 5′-OH- | 15 | UGAUCU | 371 | DTS- | 5′-PO4- | 166 | ACAGUU | 521 |
| 000404 | | | 000596 | U_{F}^{S}G_{M}^{S}A_{F}U_{M}C | | CUGGCA | | 000597 | A_{M}^{S}CE^{S}A_{M}G_{F}U | | CUGCCAG | |
| | | | | _{F}U_{M}C_{F}U_{M}G_{F}G | | GAACUG | | | _{M}U_{F}C_{M}U_{F}G_{M}C | | AGAUCAT | |
| | | | | _{M}C_{F}A_{M}G_{F}A_{M}A | | UTT | | | _{F}C_{M}A_{F}G_{M}A_{F}G | | T | |
| | | | | _{F}C_{M}U_{F}G_{M}U_{F}^{S}T | | | | | _{M}A_{F}U_{M}C_{F}A_{M}^{S}T | | | |
| | | | | _{D}^{S}T_{D}-OH-3′ | | | | | _{D}^{S}T_{D}-OH-3′ | | | |
| DT- | 316 | 334 | DTS- | 5′-OH- | 16 | GAUCUC | 357 | DTS- | 5′-PO4- | 167 | UACAGU | 590 |
| 000405 | | | 000598 | G_{F}^{S}A_{M}^{S}U_{F}C_{M}U | | UGGCAG | | 000599 | U_{M}^{S}A_{F}^{S}C_{M}A_{F}G | | UCUGCCA | |
| | | | | _{F}C_{M}U_{F}G_{M}G_{F}C | | AACUGU | | | _{M}U_{F}U_{M}C_{F}U_{M}G | | GAGAUCT | |
| | | | | _{M}A_{F}G_{M}A_{F}A_{M}C | | ATT | | | C_{M}C_{F}A_{M}G_{F}A | | T | |
| | | | | _{F}U_{M}G_{F}U_{M}A_{F}^{S}T | | | | | _{M}G_{F}A_{M}U_{F}C_{M}^{S}T | | | |
| | | | | _{D}^{S}T_{D}-OH-3′ | | | | | _{D}^{S}T_{D}-OH-3′ | | | |
| DT- | 395 | 413 | DTS- | 5′-OH- | 17 | AGUCUG | 342 | DTS- | 5′-PO4- | 168 | AUGGUG | 562 |
| 000406 | | | 000600 | A_{F}^{S}G_{M}^{S}U_{F}C_{M}U | | UCCAGGC | | 000601 | A_{M}^{S}U_{F}^{S}G_{M}G_{F}U | | GCCUGG | |
| | | | | _{F}G_{M}U_{F}C_{M}C_{F}A | | CACCAUT | | | _{M}G_{F}G_{M}C_{F}C_{M}U | | ACAGAC | |
| | | | | _{M}G_{F}G_{M}C_{F}C_{M}A | | T | | | _{F}G_{M}G_{F}A_{M}C_{F}A | | UTT | |
| | | | | _{F}C_{M}C_{F}A_{M}U_{F}^{S}T | | | | | _{M}G_{F}A_{M}C_{F}U_{M}^{S}T | | | |
| | | | | _{D}^{S}T_{D}-OH-3′ | | | | | _{D}^{S}T_{D}-OH-3′ | | | |
| DT- | 397 | 415 | DTS- | 5′-OH- | 18 | UCUGUCC | 369 | DTS- | 5′-PO4- | 169 | UCAUGG | 601 |
| 000407 | | | 000602 | U_{F}^{S}C_{M}^{S}U_{F}G_{M}U | | AGGCCAC | | 000603 | U_{M}^{S}CE^{S}A_{M}U_{F}G | | UGGCCU | |
| | | | | _{F}C_{M}C_{F}A_{M}G_{F}G | | CAUGATT | | | _{M}G_{F}U_{M}G_{F}G_{M}C | | GGACAG | |
| | | | | _{M}C_{F}C_{M}A_{F}C_{M}CE | | | | | _{F}C_{M}U_{F}G_{M}G_{F}A | | ATT | |
| | | | | A_{M}U_{F}G_{M}A_{F}^{S}T_{D} | | | | | _{M}C_{F}A_{M}G_{F}A_{M}^{S}T | | | |
| | | | | ^{S}T_{D}-OH-3′ | | | | | _{D}^{S}T_{D}-OH-3′ | | | |
| DT- | 398 | 416 | DTS- | 5′-OH- | 19 | CUGUCCA | 356 | DTS- | 5′-PO4- | 170 | AUCAUG | 548 |
| 000408 | | | 000604 | C_{F}^{S}U_{M}^{S}G_{F}U_{M}C | | GGCCACC | | 000605 | A_{M}^{S}U_{F}^{S}C_{M}A_{F}U | | GUGGCC | |
| | | | | _{F}C_{M}A_{F}G_{M}G_{F}C | | AUGAUTT | | | _{M}G_{F}G_{M}U_{F}G_{M}G | | UGGACA | |
| | | | | _{M}C_{F}A_{M}C_{F}C_{M}A_{F} | | | | | _{F}C_{M}C_{F}U_{M}G_{F}G | | GTT | |
| | | | | U_{M}G_{F}A_{M}U_{F}^{S}T_{D} | | | | | _{M}A_{F}C_{M}A_{F}G_{M}^{S}T | | | |
| | | | | ^{S}T_{D}-OH-3′ | | | | | _{D}^{S}T_{D}-OH-3′ | | | |
| DT- | 403 | 421 | DTS- | 5′-OH- | 20 | CAGGCCA | 347 | DTS- | 5′-PO4- | 171 | ACAGGA | 519 |
| 000409 | | | 000606 | C_{F}^{S}A_{M}^{S}G_{F}G_{M}C | | CCAUGA | | 000607 | A_{M}^{S}C_{F}^{S}A_{M}G_{F}G | | UCAUGG | |
| | | | | _{F}C_{M}A_{F}C_{M}C_{F}A_{M} | | UCCUGUT | | | _{M}A_{F}U_{M}C_{F}A_{M}U | | UGGCCU | |
| | | | | U_{F}G_{M}A_{F}U_{M}C_{F} | | T | | | _{F}G_{M}G_{F}U_{M}G_{F}G | | GTT | |
| | | | | C_{M}U_{F}G_{M}U_{F}^{S}T_{D} | | | | | _{M}C_{F}C_{M}U_{F}G_{M}^{S}T | | | |
| | | | | ^{S}T_{D}-OH-3′ | | | | | _{D}^{S}T_{D}-OH-3′ | | | |
| DT- | 443 | 461 | DTS- | 5′-OH- | 21 | CUCUGU | 353 | DTS- | 5′-PO4- | 172 | CAGAAG | 576 |
| 000410 | | | 000608 | C_{F}^{S}U_{M}^{S}C_{F}U_{M}G | | UCCUGU | | 000609 | C_{M}^{S}A_{F}^{S}G_{M}A_{F}A | | AACAGG | |
| | | | | _{F}U_{M}U_{F}C_{M}C_{F}U | | UCUUCU | | | _{M}G_{F}A_{M}A_{F}C_{M}A | | AACAGA | |
| | | | | _{M}G_{F}U_{M}U_{F}C_{M}U | | GTT | | | _{F}G_{M}G_{F}A_{M}A_{F}C | | GTT | |
| | | | | _{F}U_{M}C_{F}U_{M}G_{F}^{S}T | | | | | _{M}A_{F}G_{M}A_{F}G_{M}^{S} | | | |
| | | | | _{D}^{S}T_{D}-OH-3′ | | | | | T_{D}^{S}T_{D}-OH-3′ | | | |
| DT- | 444 | 462 | DTS- | 5′-OH- | 22 | UCUGUU | 370 | DTS- | 5′-PO4- | 173 | GCAGAA | 584 |
| 000411 | | | 000610 | U_{F}^{S}C_{M}^{S}U_{F}G_{M}U | | CCUGUUC | | 000611 | G_{M}^{S}C_{F}^{S}A_{M}G_{F}A | | GAACAG | |
| | | | | _{F}U_{M}C_{F}C_{M}U_{F}G | | UUCUGCT | | | _{M}A_{F}G_{M}A_{F}A_{M}C | | GAACAG | |
| | | | | _{M}U_{F}U_{M}C_{F}U_{M}U | | T | | | _{F}A_{M}G_{F}G_{M}A_{F}A | | ATT | |
| | | | | _{F}C_{M}U_{F}G_{M}CE^{S}T | | | | | _{M}C_{F}A_{M}G_{F}A_{M}^{S}T | | | |
| | | | | _{D}^{S}T_{D}-OH-3′ | | | | | _{D}^{S}T_{D}-OH-3′ | | | |
| DT- | 521 | 539 | DTS- | 5′-OH- | 23 | UUGCUG | 375 | DTS- | 5′-PO4- | 174 | AUCACGC | 546 |
| 000412 | | | 000612 | U_{F}^{S}U_{M}^{S}G_{F}C_{M}U | | GUCUGU | | 000613 | A_{M}^{S}U_{F}^{S}C_{M}A_{F}C | | ACAGACC | |
| | | | | _{F}G_{M}G_{F}U_{M}C_{F}U | | GCGUGA | | | _{M}G_{F}C_{M}A_{F}C_{M}A | | AGCAATT | |
| | | | | _{M}G_{F}U_{M}G_{F}C_{M}G | | UTT | | | _{F}G_{M}A_{F}C_{M}C_{F}A | | | |
| | | | | _{F}U_{M}G_{F}A_{M}U_{F}^{S}T | | | | | _{M}G_{F}C_{M}A_{F}A_{M}^{S}T | | | |
| | | | | _{D}^{S}T_{D}-OH-3′ | | | | | _{D}^{S}T_{D}-OH-3′ | | | |
| DT- | 525 | 543 | DTS- | 5′-OH- | 24 | UGGUCU | 373 | DTS- | 5′-PO4- | 175 | ACUCAUC | 527 |
| 000413 | | | 000614 | U_{F}^{S}G_{M}^{S}G_{F}U_{M}C | | GUGCGU | | 000615 | A_{M}^{S}C_{F}^{S}U_{M}C_{F}A | | ACGCACA | |
| | | | | _{F}U_{M}G_{F}U_{M}G_{F}C | | GAUGAG | | | _{M}U_{F}C_{M}A_{F}C_{M}G | | GACCATT | |
| | | | | _{M}G_{F}U_{M}G_{F}A_{M}U | | UTT | | | C_{M}A_{F}C_{M}A_{F}G | | | |
| | | | | _{F}G_{M}A_{F}G_{M}U_{F}^{S}T | | | | | _{M}A_{F}C_{M}C_{F}A_{M}^{S}T | | | |
| | | | | _{D}^{S}T_{D}-OH-3′ | | | | | _{D}^{S}T_{D}-OH-3′ | | | |
| DT- | 215 | 233 | DTS- | 5′-OH- | 25 | UCCUGU | 366 | DTS- | 5′-PO4- | 176 | AUGAUA | 554 |
| 000414 | | | 000616 | U_{F}^{S}C_{M}^{S}C_{F}U_{M}G | | UGCUGA | | 000617 | A_{M}^{S}U_{F}^{S}G_{M}A_{F}U | | CUCAGCA | |
| | | | | _{F}U_{M}U_{F}G_{M}C_{F}U | | GUAUCA | | | _{M}A_{F}C_{M}U_{F}C_{M}A | | ACAGGAT | |
| | | | | _{M}G_{F}A_{M}G_{F}U_{M}A | | UTT | | | _{F}G_{M}C_{F}A_{M}A_{F}C | | T | |
| | | | | _{F}U_{M}C_{F}A_{M}U_{F}^{S}T | | | | | _{M}A_{F}G_{M}G_{F}A_{M} | | | |
| | | | | _{D}^{S}T_{D}-OH-3′ | | | | | T_{D}^{S}T_{D}-OH-3′ | | | |
| DT- | 275 | 293 | DTS- | 5′-OH- | 26 | CGAUCG | 351 | DTS- | 5′-PO4- | 177 | AUCCAU | 549 |
| 000415 | | | 000618 | C_{F}^{S}G_{M}^{S}A_{F}U_{M}C | | UCAGCCA | | 000619 | A_{M}^{S}U_{F}^{S}C_{M}C_{F}A | | UGGCUG | |
| | | | | _{F}G_{M}U_{F}C_{M}A_{F}G | | AUGGAU | | | _{M}U_{F}U_{M}G_{F}G_{M}C | | ACGAUC | |
| | | | | _{M}C_{F}C_{M}A_{F}A_{M}U | | TT | | | _{F}U_{M}G_{F}A_{M}C_{F}G | | GTT | |
| | | | | _{F}G_{M}G_{F}A_{M}U_{F}^{S}T | | | | | _{M}A_{F}U_{M}C_{F}G_{M}^{S}T | | | |
| | | | | _{D}^{S}T_{D}-OH-3′ | | | | | _{D}^{S}T_{D}-OH-3′ | | | |
| DT- | 325 | 343 | DTS- | 5′-OH- | 27 | CAGAAC | 346 | DTS- | 5′-PO4- | 178 | AAGAGG | 502 |
| 000416 | | | 000620 | C_{F}^{S}A_{M}^{S}G_{F}A_{M}A | | UGUAGC | | 000621 | A_{M}^{S}A_{F}^{S}G_{M}A_{F}G | | UGCUAC | |
| | | | | _{F}C_{M}U_{F}G_{M}U_{F}A | | ACCUCUU | | | _{M}G_{F}U_{M}G_{F}C_{M}U | | AGUUCU | |
| | | | | _{M}G_{F}C_{M}A_{F}C_{M}C_{F} | | TT | | | _{F}A_{M}C_{F}A_{M}G_{F}U | | GTT | |
| | | | | U_{M}C_{F}U_{M}U_{F}^{S}T_{D} | | | | | _{M}U_{F}C_{M}U_{F}G_{M}^{S}T | | | |
| | | | | ^{S}T_{D}-OH-3′ | | | | | _{D}^{S}T_{D}-OH-3′ | | | |
| DT- | 338 | 356 | DTS- | 5′-OH- | 28 | CCUCUUC | 350 | DTS- | 5′-PO4- | 179 | ACAUUU | 522 |
| 000417 | | | 000622 | C_{F}^{S}C_{M}^{S}U_{F}C_{M}U | | CUCAGG | | 000623 | A_{M}^{S}C_{F}^{S}A_{M}U_{F}U | | CCUGAG | |
| | | | | _{F}U_{M}C_{F}C_{M}U_{F}C_{M} | | AAAUGU | | | _{M}U_{F}C_{M}C_{F}U_{M}G | | GAAGAG | |
| | | | | A_{F}G_{M}G_{F}A_{M}A_{F} | | TT | | | _{F}A_{M}G_{F}G_{M}A_{F}A | | GTT | |
| | | | | A_{M}U_{F}G_{M}U_{F}^{S}T_{D} | | | | | _{M}G_{F}A_{M}G_{F}G_{M}′^{S} | | | |
| | | | | ^{S}T_{D}-OH-3′ | | | | | T_{D}^{S}T_{D}-OH-3′ | | | |
| DT- | 370 | 388 | DTS- | 5′-OH- | 29 | UCAUCA | 365 | DTS- | 5′-PO4- | 180 | AUUCGU | 569 |
| 000418 | | | 000624 | U_{F}^{S}C_{M}^{S}A_{F}U_{M}C | | UCACCAA | | 000625 | A_{M}^{S}U_{F}^{S}U_{M}C_{F}G | | UUGGUG | |
| | | | | _{F}A_{M}U_{F}C_{M}A_{F}C | | ACGAAUT | | | _{M}U_{F}U_{M}U_{F}G_{M}G | | AUGAUG | |
| | | | | _{M}C_{F}A_{M}A_{F}A_{M}C | | T | | | _{F}U_{M}G_{F}A_{M}U_{F}G | | ATT | |
| | | | | G_{M}A_{F}A_{M}U_{F}^{S}T | | | | | _{M}A_{F}U_{M}G_{F}A_{M}^{S} | | | |
| | | | | _{D}^{S}T_{D}-OH-3′ | | | | | T_{D}^{S}T_{D}-OH-3′ | | | |
| DT- | 410 | 428 | DTS- | 5′-OH- | 30 | CCAUGA | 349 | DTS- | 5′-PO4- | 181 | AUGAUC | 555 |
| 000419 | | | 000626 | C_{F}^{S}C_{M}^{S}A_{F}U_{M}G | | UCCUGUC | | 000627 | A_{M}^{S}U_{F}^{S}G_{M}A_{F}U | | GACAGG | |
| | | | | _{F}A_{M}U_{F}C_{M}C_{F}U | | GAUCAUT | | | _{M}C_{F}G_{M}A_{F}C_{M}A | | AUCAUG | |
| | | | | _{M}G_{F}U_{M}C_{F}G_{M}A | | T | | | G_{M}G_{F}A_{M}U_{F}C | | GTT | |
| | | | | _{F}U_{M}C_{F}A_{M}U_{F}^{S}T | | | | | _{M}A_{F}U_{M}G_{F}G_{M}^{S} | | | |
| | | | | _{D}^{S}T_{D}-OH-3′ | | | | | T_{D}^{S}T_{D}-OH-3′ | | | |
| DT- | 431 | 449 | DTS- | 5′-OH- | 31 | UCAGCA | 364 | DTS- | 5′-PO4- | 182 | AACAGA | 498 |
| 000420 | | | 000628 | U_{F}^{S}C_{M}^{S}A_{F}G_{M}C | | UUCUGU | | 000629 | A_{M}^{S}A_{F}^{S}C_{M}A_{F}G | | GACAGA | |
| | | | | _{F}A_{M}U_{F}U_{M}C_{F}U | | CUCUGU | | | _{M}A_{F}G_{M}A_{F}C_{M}A | | AUGCUG | |
| | | | | _{M}G_{F}U_{M}C_{F}U_{M}C | | UTT | | | _{F}G_{M}A_{F}A_{M}U_{F}G | | | |
| | | | | _{F}U_{M}G_{F}U_{M}U_{F}^{S}T | | | | | _{M}C_{F}U_{M}G_{F}A_{M}^{S}T | | | |
| | | | | _{D}^{S}T_{D}-OH-3′ | | | | | _{D}^{S}T_{D}-OH-3′ | | ATT | |
| DT- | 442 | 460 | DTS- | 5′-OH- | 32 | UCUCUG | 368 | DTS- | 5′-PO4- | 183 | AGAAGA | 529 |
| 000421 | | | 000630 | U_{F}^{S}C_{M}^{S}U_{F}C_{M}U | | UUCCUG | | 000631 | A_{M}^{S}GE^{S}A_{M}A_{F}G | | ACAGGA | |
| | | | | _{F}G_{M}U_{F}U_{M}C_{F}C | | UUCUUC | | | _{M}A_{F}A_{M}C_{F}A_{M}G | | ACAGAG | |
| | | | | _{M}U_{F}G_{M}U_{F}U_{M}C | | UTT | | | G_{M}A_{F}A_{M}C_{F}A | | ATT | |
| | | | | _{F}U_{M}U_{F}C_{M}U_{F}^{S}T | | | | | _{M}G_{F}A_{M}G_{F}A_{M}^{S} | | | |
| | | | | _{D}^{S}T_{D}-OH-3′ | | | | | T_{D}^{S}T_{D}-OH-3′ | | | |
| DT- | 500 | 518 | DTS- | 5′-OH- | 33 | UCACUG | 363 | DTS- | 5′-PO4- | 184 | AUUUGG | 575 |
| 000422 | | | 000632 | U_{F}^{S}C_{M}^{S}A_{F}C_{M}U | | GAAUCU | | 000633 | A_{M}^{S}U_{F}^{S}U_{M}U_{F}G | | AAGAUU | |
| | | | | G_{M}G_{F}A_{M}A_{F}U | | UCCAAA | | | _{M}G_{F}A_{M}A_{F}G_{M}A | | CCAGUG | |
| | | | | _{M}C_{F}U_{M}U_{F}C_{M}C_{F} | | UTT | | | _{F}U_{M}U_{F}C_{M}C_{F}A | | ATT | |
| | | | | A_{M}A_{F}A_{M}U_{F}^{S}T_{D} | | | | | _{M}G_{F}U_{M}G_{F}A_{M}^{S} | | | |
| | | | | ^{S}T_{D}-OH-3′ | | | | | T_{D}^{S}T_{D}-OH-3′ | | | |
| DT- | 503 | 521 | DTS- | 5′-OH- | 34 | CUGGAA | 355 | DTS- | 5′-PO4- | 185 | AGAAUU | 530 |
| 000423 | | | 000634 | C_{F}^{S}U_{M}^{S}G_{F}G_{M}A | | UCUUCCA | | 000635 | A_{M}^{S}G_{F}^{S}A_{M}A_{F}U | | UGGAAG | |
| | | | | _{F}A_{M}U_{F}C_{M}U_{F}U | | AAUUCUT | | | _{M}U_{F}U_{M}G_{F}G_{M}A | | AUUCCA | |
| | | | | _{M}C_{F}C_{M}A_{F}A_{M}A | | T | | | _{F}A_{M}G_{F}A_{M}U_{F}U | | GTT | |
| | | | | _{F}U_{M}U_{F}C_{M}U_{F}^{S}T | | | | | _{M}C_{F}C_{M}A_{F}G_{M}^{S}T | | | |
| | | | | _{D}T_{D}-OH-3′ | | | | | _{D}^{S}T_{D}-OH-3′ | | | |
| DT- | 647 | 665 | DTS- | 5′-OH- | 35 | UUCUCA | 374 | DTS- | 5′-PO4- | 186 | UAGAUG | 593 |
| 000424 | | | 000636 | U_{F}^{S}U_{M}C_{F}U_{M}C | | GCGGUG | | 000637 | U_{M}^{S}A_{F}^{S}G_{M}A_{F}U | | ACACCGC | |
| | | | | _{F}A_{M}G_{F}C_{M}G_{F}G | | UCAUCU | | | _{M}G_{F}A_{M}C_{F}A_{M}C | | UGAGAA | |
| | | | | _{M}U_{F}G_{M}U_{F}C_{M}A | | ATT | | | C_{M}G_{F}C_{M}U_{F}G | | TT | |
| | | | | _{F}U_{M}C_{F}U_{M}A_{F}^{S}T | | | | | _{M}A_{F}G_{M}A_{F}A_{M}^{S} | | | |
| | | | | _{D}^{S}T_{D}-OH-3′ | | | | | T_{D}^{S}T_{D}-OH-3′ | | | |
| DT- | 648 | 666 | DTS- | 5′-OH- | 36 | UCUCAGC | 367 | DTS- | 5′-PO4- | 187 | AUAGAU | 543 |
| 000425 | | | 000638 | U_{F}^{S}C_{M}^{S}U_{F}C_{M}A | | GGUGUC | | 000639 | A_{M}^{S}U_{F}^{S}A_{M}G_{F}A | | GACACCG | |
| | | | | _{F}G_{M}C_{F}G_{M}G_{F}U | | AUCUAUT | | | _{M}U_{F}G_{M}A_{F}C_{M}A | | CUGAGAT | |
| | | | | _{M}G_{F}U_{M}C_{F}A_{M}U | | T | | | _{F}C_{M}C_{F}G_{M}C_{F}U_{M} | | T | |
| | | | | _{F}C_{M}U_{F}A_{M}U_{F}^{S}T | | | | | G_{F}A_{M}G_{F}A_{M}^{S}T_{D} | | | |
| | | | | _{D}^{S}T_{D}-OH-3′ | | | | | ^{S}T_{D}-OH-3′ | | | |
| DT- | 210 | 228 | DTS- | 5′-OH- | 37 | GCUCCUC | 376 | DTS- | 5′-PO4- | 189 | ACUCAGC | 526 |
| 000845 | | | 001263 | G_{F}^{S}C_{M}^{S}U_{F}C_{M}C | | CUGUUG | | 001264 | A_{M}^{S}C_{F}^{S}U_{M}C_{F}A | | AACAGG | |
| | | | | _{F}U_{M}C_{F}C_{M}U_{F}G | | CUGAGUT | | | _{M}G_{F}C_{M}A_{F}A_{M}C | | AGGAGCT | |
| | | | | _{M}U_{F}U_{M}G_{F}C_{M}U | | T | | | _{F}A_{M}G_{F}G_{M}A_{F}G | | T | |
| | | | | _{F}G_{M}A_{F}G_{M}U_{F}^{S}T | | | | | _{M}G_{F}A_{M}G_{F}C_{M}^{S}T | | | |
| | | | | _{D}^{S}T_{D}-OH-3′ | | | | | _{D}^{S}T_{D}-OH-3′ | | | |
| DT- | 212 | 230 | DTS- | 5′-OH- | 38 | UCCUCCU | 377 | DTS- | 5′-PO4- | 190 | AUACUC | 542 |
| 000846 | | | 001265 | U_{F}^{S}C_{M}^{S}C_{F}U_{M}C | | GUUGCU | | 001266 | A_{M}^{S}U_{F}^{S}A_{M}C_{F}U | | AGCAAC | |
| | | | | _{F}C_{M}U_{F}G_{M}U_{F}U | | GAGUAU | | | _{M}C_{F}A_{M}G_{F}C_{M}A | | AGGAGG | |
| | | | | _{M}G_{F}C_{M}U_{F}G_{M}A | | TT | | | _{F}A_{M}C_{F}A_{M}G_{F}G | | ATT | |
| | | | | _{F}G_{M}U_{F}A_{M}U_{F}^{S}T | | | | | _{M}A_{F}G_{M}G_{F}A_{M}^{S} | | | |
| | | | | _{D}^{S}T_{D}-OH-3′ | | | | | T_{D}^{S}T_{D}-OH-3′ | | | |
| DT- | 213 | 231 | DTS- | 5′-OH- | 39 | CCUCCUG | 378 | DTS- | 5′-PO4- | 191 | GAUACU | 582 |
| 000847 | | | 001267 | C_{F}^{S}C_{M}^{S}U_{F}C_{M}C | | UUGCUG | | 001268 | G_{M}^{S}A_{F}^{S}U_{M}A_{F}C | | CAGCAAC | |
| | | | | _{F}U_{M}G_{F}U_{M}U_{F}G | | AGUAUCT | | | _{M}U_{F}C_{M}A_{F}G_{M}C | | AGGAGG | |
| | | | | _{M}C_{F}U_{M}G_{F}A_{M}G | | T | | | _{F}A_{M}A_{F}C_{M}A_{F}G | | TT | |
| | | | | _{F}U_{M}A_{F}U_{M}CE^{S}T | | | | | _{M}G_{F}A_{M}G_{F}G_{M}^{S} | | | |
| | | | | _{D}^{S}T_{D}-OH-3′ | | | | | T_{D}^{S}T_{D}-OH-3′ | | | |
| DT- | 214 | 232 | DTS- | 5′-OH- | 40 | CUCCUGU | 379 | DTS- | 5′-PO4- | 192 | UGAUAC | 605 |
| 000848 | | | 001269 | C_{F}^{S}U_{M}^{S}C_{F}C_{M}U | | UGCUGA | | 001270 | U_{M}^{S}G_{F}^{S}A_{M}U_{F}A | | UCAGCA | |
| | | | | _{F}G_{M}U_{F}U_{M}G_{F}C | | GUAUCAT | | | _{M}C_{F}U_{M}C_{F}A_{M}G | | ACAGGA | |
| | | | | _{M}U_{F}G_{M}A_{F}G_{M}U | | T | | | _{F}C_{M}A_{F}A_{M}C_{F}A | | GTT | |
| | | | | _{F}A_{M}U_{F}C_{M}A_{F}^{S}T | | | | | _{M}G_{F}G_{M}A_{F}G_{M} | | | |
| | | | | _{D}^{S}T_{D}-OH-3′ | | | | | T_{D}^{S}T_{D}-OH-3′ | | | |
| DT- | 216 | 234 | DTS- | 5′-OH- | 41 | CCUGUU | 380 | DTS- | 5′-PO4- | 193 | GAUGAU | 583 |
| 000849 | | | 001271 | C_{F}^{S}C_{M}^{S}U_{F}G_{M}U | | GCUGAG | | 001272 | G_{M}^{S}A_{F}^{S}U_{M}G_{F}A | | ACUCAGC | |
| | | | | _{F}U_{M}G_{F}C_{M}U_{F}G | | UAUCAU | | | _{M}U_{F}A_{M}C_{F}U_{M}C | | AACAGGT | |
| | | | | _{M}A_{F}G_{M}U_{F}A_{M}U | | CTT | | | _{F}A_{M}G_{F}C_{M}A_{F}A | | T | |
| | | | | _{F}C_{M}A_{F}U_{M}C_{F}^{S}T | | | | | _{M}C_{F}A_{M}G_{F}G_{M}^{S}T | | | |
| | | | | _{D}^{S}T_{D}-OH-3′ | | | | | _{D}^{S}T_{D}-OH-3′ | | | |
| DT- | 217 | 235 | DTS- | 5′-OH- | 42 | CUGUUG | 381 | DTS- | 5′-PO4- | 194 | CGAUGA | 579 |
| 000850 | | | 001273 | C_{F}^{S}U_{M}^{S}G_{F}U_{M}U | | CUGAGU | | 001274 | C_{M}^{S}GE^{S}A_{M}U_{F}G | | UACUCA | |
| | | | | _{F}G_{M}C_{F}U_{M}G_{F}A | | AUCAUC | | | _{M}A_{F}U_{M}A_{F}C_{M}U | | GCAACA | |
| | | | | _{M}G_{F}U_{M}A_{F}U_{M}C | | GTT | | | _{F}C_{M}A_{F}G_{M}C_{F}A | | GTT | |
| | | | | _{F}A_{M}U_{F}C_{M}GE^{S}T | | | | | _{M}A_{F}C_{M}A_{F}G_{M}^{S}T | | | |
| | | | | _{D}^{S}T_{D}-OH-3′ | | | | | _{D}^{S}T_{D}-OH-3′ | | | |
| DT- | 218 | 236 | DTS- | 5′-OH- | 43 | UGUUGC | 382 | DTS- | 5′-PO4- | 195 | ACGAUG | 523 |
| 000851 | | | 001275 | U_{F}^{S}G_{M}^{S}U_{F}U_{M}G | | UGAGUA | | 001276 | A_{M}^{S}C_{F}^{S}G_{M}A_{F}U | | AUACUC | |
| | | | | _{F}C_{M}U_{F}G_{M}A_{F}G | | UCAUCG | | | _{M}G_{F}A_{M}U_{F}A_{M}C | | AGCAAC | |
| | | | | _{M}U_{F}A_{M}U_{F}C_{M}A | | UTT | | | _{F}U_{M}C_{F}A_{M}G_{F}C | | ATT | |
| | | | | _{F}U_{M}C_{F}G_{M}U_{F}^{S}T | | | | | _{M}A_{F}A_{M}C_{F}A_{M}^{S}T | | | |
| | | | | _{D}^{S}T_{D}-OH-3′ | | | | | _{D}^{S}T_{D}-OH-3′ | | | |
| DT- | 219 | 237 | DTS- | 5′-OH- | 44 | GUUGCU | 383 | DTS- | 5′-PO4- | 196 | GACGAU | 581 |
| 000852 | | | 001277 | G_{F}^{S}U_{M}^{S}U_{F}G_{M}C | | GAGUAU | | 001278 | G_{M}^{S}A_{F}^{S}C_{M}G_{F}A | | GAUACU | |
| | | | | _{F}U_{M}G_{F}A_{M}G_{F}U | | CAUCGUC | | | _{M}U_{F}G_{M}A_{F}U_{M}A | | CAGCAAC | |
| | | | | _{M}A_{F}U_{M}C_{F}A_{M}U | | TT | | | _{F}C_{M}U_{F}C_{M}A_{F}G | | TT | |
| | | | | C_{M}G_{F}U_{M}C_{F}^{S}T | | | | | _{M}C_{F}A_{M}A_{F}C_{M}^{S}T | | | |
| | | | | _{D}^{S}T_{D}-OH-3′ | | | | | _{D}^{S}T_{D}-OH-3′ | | | |
| DT- | 220 | 238 | DTS- | 5′-OH- | 45 | UUGCUG | 384 | DTS- | 5′-PO4- | 197 | GGACGA | 585 |
| 000853 | | | 001279 | U_{F}^{S}U_{M}^{S}G_{F}C_{M}U | | AGUAUC | | 001280 | G_{M}^{S}G_{F}^{S}A_{M}C_{F}G | | UGAUAC | |
| | | | | _{F}G_{M}A_{F}G_{M}U_{F}A | | AUCGUCC | | | _{M}A_{F}U_{M}G_{F}A_{M}U | | UCAGCA | |
| | | | | _{M}U_{F}C_{M}A_{F}U_{M}C | | TT | | | _{F}A_{M}C_{F}U_{M}C_{F}A | | ATT | |
| | | | | _{F}G_{M}U_{F}C_{M}C_{F}^{S}T | | | | | _{M}G_{F}C_{M}A_{F}A_{M}^{S}T | | | |
| | | | | _{D}^{S}T_{D}-OH-3′ | | | | | _{D}^{S}T_{D}-OH-3′ | | | |
| DT- | 224 | 242 | DTS- | 5′-OH- | 46 | UGAGUA | 385 | DTS- | 5′-PO4- | 198 | UGGAGG | 608 |
| 000854 | | | 001281 | U_{F}^{S}G_{M}^{S}A_{F}G_{M}U | | UCAUCG | | 001282 | U_{M}^{S}G_{F}^{S}G_{M}A_{F}G | | ACGAUG | |
| | | | | _{F}A_{M}U_{F}C_{M}A_{F}U | | UCCUCCA | | | _{M}G_{F}A_{M}C_{F}G_{M}A | | AUACUC | |
| | | | | _{M}C_{F}G_{M}U_{F}C_{M}C_{F} | | TT | | | _{F}U_{M}G_{F}A_{M}U_{F}A | | ATT | |
| | | | | U_{M}C_{F}C_{M}A_{F}^{S}T_{D} | | | | | _{M}C_{F}U_{M}C_{F}A_{M}^{S}T | | | |
| | | | | ^{S}T_{D}-OH-3′ | | | | | _{D}^{S}T_{D}-OH-3′ | | | |
| DT- | 227 | 245 | DTS- | 5′-OH- | 47 | GUAUCA | 386 | DTS- | 5′-PO4- | 199 | ACGUGG | 524 |
| 000855 | | | 001283 | G_{F}^{S}U_{M}^{S}A_{F}U_{M}C | | UCGUCCU | | 001284 | A_{M}^{S}C_{F}^{S}G_{M}U_{F}G | | AGGACG | |
| | | | | _{F}A_{M}U_{F}C_{M}G_{F}U | | CCACGUT | | | _{M}G_{F}A_{M}G_{F}G_{M}A | | AUGAUA | |
| | | | | _{M}C_{F}C_{M}U_{F}C_{M}C_{F} | | T | | | _{F}C_{M}G_{F}A_{M}U_{F}G | | CTT | |
| | | | | A_{M}C_{F}G_{M}U_{F}^{S}T_{D} | | | | | _{M}A_{F}U_{M}A_{F}C_{M}^{S}T | | | |
| | | | | ^{S}T_{D}-OH-3′ | | | | | _{D}^{S}T_{D}-OH-3′ | | | |
| DT- | 245 | 263 | DTS- | 5′-OH- | 48 | UCGCGG | 387 | DTS- | 5′-PO4- | 200 | AGCAGC | 534 |
| 000856 | | | 001285 | U_{F}^{S}C_{M}^{S}G_{F}C_{M}G | | UGCUGG | | 001286 | A_{M}^{S}G_{F}^{S}C_{M}A_{F}G | | ACCAGCA | |
| | | | | _{F}G_{M}U_{F}G_{M}C_{F}U | | UGCUGC | | | _{M}C_{F}A_{M}C_{F}C_{M}A_{F} | | CCGCGAT | |
| | | | | _{M}G_{F}G_{M}U_{F}G_{M}C | | UTT | | | G_{M}C_{F}A_{M}C_{F}C_{M} | | T | |
| | | | | _{F}U_{M}G_{F}C_{M}U_{F}^{S}T | | | | | G_{F}C_{M}G_{F}A_{M}^{S}T_{D} | | | |
| | | | | _{D}^{S}T_{D}-OH-3′ | | | | | ^{S}T_{D}-OH-3′ | | | |
| DT- | 248 | 266 | DTS- | 5′-OH- | 49 | CGGUGC | 388 | DTS- | 5′-PO4- | 201 | AACAGC | |
| 000857 | | | 001287 | C_{F}^{S}G_{M}^{S}G_{F}U_{M}G | | UGGUGC | | 001288 | A_{M}^{S}A_{F}^{S}C_{M}A_{F}G | | AGCACCA | |
| | | | | _{F}C_{M}U_{F}G_{M}G_{F}U | | UGCUGU | | | _{M}C_{F}A_{M}G_{F}C_{M}A | | GCACCGT | |
| | | | | _{M}G_{F}C_{M}U_{F}G_{M}C | | UTT | | | _{F}C_{M}C_{F}A_{M}G_{F}C_{M} | | T | |
| | | | | _{F}U_{M}G_{F}U_{M}U_{F}^{S}T | | | | | A_{F}C_{M}C_{F}G_{M}^{S}T_{D} | | | |
| | | | | _{D}^{S}T_{D}-OH-3′ | | | | | ^{S}T_{D}-OH-3′ | | | 499 |
| DT- | 253 | 271 | DTS- | 5′-OH- | 50 | CUGGUG | 389 | DTS- | 5′-PO4- | 202 | AGACGA | |
| 000858 | | | 001289 | C_{F}^{S}U_{M}^{S}G_{F}G_{M}U | | CUGCUG | | 001290 | A_{M}^{S}G_{F}^{S}A_{M}C_{F}G | | ACAGCA | |
| | | | | _{F}G_{M}C_{F}U_{M}G_{F}C | | UUCGUC | | | _{M}A_{F}A_{M}C_{F}A_{M}G | | GCACCAG | |
| | | | | _{M}U_{F}G_{M}U_{F}U_{M}C | | UTT | | | _{F}C_{M}A_{F}G_{M}C_{F}A | | TT | |
| | | | | _{F}G_{M}U_{F}C_{M}U_{F}^{S}T | | | | | _{M}C_{F}C_{M}A_{F}G_{M}^{S}T | | | |
| | | | | _{D}^{S}T_{D}-OH-3′ | | | | | _{D}T_{D}-OH-3′ | | | 531 |
| DT- | 256 | 274 | DTS- | 5′-OH- | 51 | GUGCUG | 390 | DTS- | 5′-PO4- | 203 | UGGAGA | 607 |
| 000859 | | | 001291 | G_{F}^{S}U_{M}^{S}G_{F}C_{M}U | | CUGUUC | | 001292 | U_{M}^{S}G_{F}^{S}G_{M}A_{F}G | | CGAACA | |
| | | | | _{F}G_{M}C_{F}U_{M}G_{F}U | | GUCUCCA | | | _{M}A_{F}C_{M}G_{F}A_{M}A | | GCAGCAC | |
| | | | | _{M}U_{F}C_{M}G_{F}U_{M}C | | TT | | | _{F}C_{M}A_{F}G_{M}C_{F}A | | TT | |
| | | | | _{F}U_{M}C_{F}C_{M}A_{F}^{S}T | | | | | _{M}G_{F}C_{M}A_{F}C_{M}^{S}T | | | |
| | | | | _{D}^{S}T_{D}-OH-3′ | | | | | _{D}^{S}T_{D}-OH-3′ | | | |
| DT- | 260 | 278 | DTS- | 5′-OH- | 52 | UGCUGU | 391 | DTS- | 5′-PO4- | 204 | AUCGUG | 551 |
| 000860 | | | 001293 | U_{F}^{S}G_{M}^{S}C_{F}U_{M}G | | UCGUCUC | | 001294 | A_{M}^{S}U_{F}^{S}C_{M}G_{F}U | | GAGACG | |
| | | | | _{F}U_{M}U_{F}C_{M}G_{F}U | | CACGAUT | | | _{M}G_{F}G_{M}A_{F}G_{M}A | | AACAGC | |
| | | | | _{M}C_{F}U_{M}C_{F}C_{M}A_{F} | | T | | | _{F}C_{M}G_{F}A_{M}A_{F}C | | ATT | |
| | | | | C_{M}G_{F}A_{M}U_{F}^{S}T_{D} | | | | | _{M}A_{F}G_{M}C_{F}A_{M}^{S}T | | | |
| | | | | ^{S}T_{D}-OH-3′ | | | | | _{D}^{S}T_{D}-OH-3′ | | | |
| DT- | 265 | 283 | DTS- | 5′-OH- | 53 | UUCGUC | 392 | DTS- | 5′-PO4- | 205 | UGACGA | 603 |
| 000861 | | | 001295 | U_{F}^{S}U_{M}C_{F}G_{M}U | | UCCACGA | | 001296 | U_{M}^{S}G_{F}^{S}A_{M}C_{F}G | | UCGUGG | |
| | | | | C_{M}U_{F}C_{M}C_{F}A_{M} | | UCGUCAT | | | _{M}A_{F}U_{M}C_{F}G_{M}U | | AGACGA | |
| | | | | C_{F}G_{M}A_{F}U_{M}C_{F} | | T | | | _{F}G_{M}G_{F}A_{M}G_{F}A | | ATT | |
| | | | | G_{M}U_{F}C_{M}A_{F}^{S}T_{D} | | | | | _{M}C_{F}G_{M}A_{F}A_{M}^{S}T | | | |
| | | | | ^{S}T_{D}-OH-3′ | | | | | _{D}^{S}T_{D}-OH-3′ | | | |
| DT- | 271 | 289 | DTS- | 5′-OH- | 54 | UCCACGA | 393 | DTS- | 5′-PO4- | 206 | AUUGGC | 572 |
| 000862 | | | 001297 | U_{F}^{S}C_{M}^{S}C_{F}A_{M}C | | UCGUCA | | 001298 | A_{M}^{S}U_{F}^{S}U_{M}G_{F}G | | UGACGA | |
| | | | | _{F}G_{M}A_{F}U_{M}C_{F}G | | GCCAAUT | | | _{M}C_{F}U_{M}G_{F}A_{M}C | | UCGUGG | |
| | | | | _{M}U_{F}C_{M}A_{F}G_{M}C | | T | | | _{F}G_{M}A_{F}U_{M}C_{F}G | | ATT | |
| | | | | _{F}C_{M}A_{F}A_{M}U_{F}^{S}T | | | | | _{M}U_{F}G_{M}G_{F}A_{M}^{S} | | | |
| | | | | _{D}^{S}T_{D}-OH-3′ | | | | | T_{D}^{S}T_{D}-OH-3′ | | | |
| DT- | 284 | 302 | DTS- | 5′-OH- | 55 | GCCAAU | 394 | DTS- | 5′-PO4- | 207 | UUGCCCA | 625 |
| 000863 | | | 001299 | G_{F}^{S}C_{M}^{S}C_{F}A_{M}A | | GGAUCG | | 001300 | U_{M}^{S}U_{F}^{S}G_{M}C_{F}C | | CGAUCCA | |
| | | | | _{F}U_{M}G_{F}G_{M}A_{F}U | | UGGGCA | | | _{M}C_{F}A_{M}C_{F}G_{M}A | | UUGGCTT | |
| | | | | _{M}C_{F}G_{M}U_{F}G_{M}G | | ATT | | | _{F}U_{M}C_{F}C_{M}A_{F}U | | | |
| | | | | _{F}G_{M}C_{F}A_{M}A_{F}^{S}T | | | | | _{M}U_{F}G_{M}G_{F}C_{M}^{S}T | | | |
| | | | | _{D}^{S}T_{D}-OH-3′ | | | | | _{D}^{S}T_{D}-OH-3′ | | | |
| DT- | 295 | 313 | DTS- | 5′-OH- | 56 | GUGGGC | 395 | DTS- | 5′-PO4- | 208 | UUGCGU | 626 |
| 000864 | | | 001301 | G_{F}^{S}U_{M}^{S}G_{F}G_{M}G | | AAUGGA | | 001302 | U_{M}^{S}U_{F}^{S}G_{M}C_{F}G | | GUCCAU | |
| | | | | C_{M}A_{F}A_{M}U_{F}G | | CACGCAA | | | _{M}U_{F}G_{M}U_{F}C_{M}C | | UGCCCAC | |
| | | | | _{M}G_{F}A_{M}C_{F}A_{M}C | | TT | | | _{F}A_{M}U_{F}U_{M}G_{F}C | | TT | |
| | | | | _{F}G_{M}C_{F}A_{M}A_{F}^{S}T | | | | | _{M}C_{F}C_{M}A_{F}C_{M}^{S}T | | | |
| | | | | _{D}^{S}T_{D}-OH-3′ | | | | | _{D}^{S}T_{D}-OH-3′ | | | |
| DT- | 300 | 318 | DTS- | 5′-OH- | 57 | CAAUGG | 396 | DTS- | 5′-PO4- | 209 | AUCAGU | 547 |
| 000865 | | | 001303 | C_{F}^{S}A_{M}^{S}A_{F}U_{M}G | | ACACGCA | | 001304 | A_{M}^{S}U_{F}^{S}C_{M}A_{F}G | | UGCGUG | |
| | | | | _{F}G_{M}A_{F}C_{M}A_{F}C | | ACUGAUT | | | _{M}U_{F}U_{M}G_{F}C_{M}G | | UCCAUU | |
| | | | | _{M}G_{F}C_{M}A_{F}A_{M}C | | T | | | _{F}U_{M}G_{F}U_{M}C_{F}C | | GTT | |
| | | | | _{F}U_{M}G_{F}A_{M}U_{F}^{S}T | | | | | _{M}A_{F}U_{M}U_{F}G_{M}^{S} | | | |
| | | | | _{D}^{S}T_{D}-OH-3′ | | | | | T_{D}^{S}T_{D}-OH-3′ | | | |
| DT- | 304 | 322 | DTS- | 5′-OH- | 58 | GGACAC | 397 | DTS- | 5′-PO4- | 210 | AGAGAU | 532 |
| 000866 | | | 001305 | G_{F}^{S}G_{M}^{S}A_{F}C_{M}A | | GCAACU | | 001306 | A_{M}^{S}G_{F}^{S}A_{M}G_{F}A | | CAGUUG | |
| | | | | _{F}C_{M}G_{F}C_{M}A_{F}A | | GAUCUC | | | _{M}U_{F}C_{M}A_{F}G_{M}U | | CGUGUCC | |
| | | | | _{M}C_{F}U_{M}G_{F}A_{M}U | | UTT | | | _{F}U_{M}G_{F}C_{M}G_{F}U | | TT | |
| | | | | C_{M}U_{F}C_{M}U_{F}^{S}T | | | | | _{M}G_{F}U_{M}C_{F}C_{M}^{S}T | | | |
| | | | | _{D}^{S}T_{D}-OH-3′ | | | | | _{D}^{S}T_{D}-OH-3′ | | | |
| DT- | 335 | 353 | DTS- | 5′-OH- | 59 | GCACCUC | 398 | DTS- | 5′-PO4- | 211 | UUUCCU | 632 |
| 000867 | | | | G_{F}^{S}C_{M}^{S}A_{F}C_{M}C | | UUCCUCA | | 001308 | U_{M}^{S}U_{F}^{S}U_{M}C_{F}C | | GAGGAA | |
| | | | | _{F}U_{M}C_{F}U_{M}U_{F}C | | GGAAATT | | | _{M}U_{F}G_{M}A_{F}G_{M}G | | GAGGUG | |
| | | | 001307 | _{M}C_{F}U_{M}C_{F}A_{M}G | | | | | _{F}A_{M}A_{F}G_{M}A_{F}G | | CTT | |
| | | | | _{F}G_{M}A_{F}A_{M}A_{F}^{S}T | | | | | _{M}G_{F}U_{M}G_{F}C_{M}^{S}T | | | |
| | | | | _{D}^{S}T_{D}-OH-3′ | | | | | _{D}^{S}T_{D}-OH-3′ | | | |
| DT- | 350 | 368 | DTS- | 5′-OH- | 60 | GAAAUG | 399 | DTS- | 5′-PO4- | 212 | AAACAG | 492 |
| 000868 | | | 001309 | G_{F}^{S}A_{M}^{S}A_{F}A_{M}U | | UCCACCA | | 001310 | A_{M}^{S}A_{F}^{S}A_{M}C_{F}A | | UGGUGG | |
| | | | | _{F}G_{M}U_{F}C_{M}C_{F}A | | CUGUUUT | | | _{M}G_{F}U_{M}G_{F}G_{M}U | | ACAUUU | |
| | | | | _{M}C_{F}C_{M}A_{F}C_{M}U_{F} | | T | | | G_{M}G_{F}A_{M}C_{F}A | | CTT | |
| | | | | G_{M}U_{F}U_{M}U_{F}^{S}T_{D} | | | | | _{M}U_{F}U_{M}U_{F}C_{M}^{S}T | | | |
| | | | | ^{S}T_{D}-OH-3′ | | | | | _{D}^{S}T_{D}-OH-3′ | | | |
| DT- | 358 | 376 | DTS- | 5′-OH- | 61 | CACCACU | 400 | DTS- | 5′-PO4- | 213 | AUGAUG | 556 |
| 000869 | | | 001311 | C_{F}^{S}A_{M}^{S}C_{F}C_{M}A | | GUUUCU | | 001312 | A_{M}^{S}U_{F}^{S}G_{M}A_{F}U | | AGAAAC | |
| | | | | _{F}C_{M}U_{F}G_{M}U_{F}U | | CAUCAUT | | | _{M}G_{F}A_{M}G_{F}A_{M}A | | AGUGGU | |
| | | | | _{M}U_{F}C_{M}U_{F}C_{M}A | | T | | | _{F}A_{M}C_{F}A_{M}G_{F}U | | GTT | |
| | | | | _{F}U_{M}C_{F}A_{M}U_{F}^{S}T | | | | | _{M}G_{F}G_{M}U_{F}G_{M}^{S} | | | |
| | | | | _{D}^{S}T_{D}-OH-3′ | | | | | T_{D}^{S}T_{D}-OH-3′ | | | |
| DT- | 364 | 382 | DTS- | 5′-OH- | 62 | UGUUUC | 401 | DTS- | 5′-PO4- | 214 | UUGGUG | 629 |
| 000870 | | | 001313 | U_{F}^{S}G_{M}^{S}U_{F}U_{M}U | | UCAUCA | | 001314 | U_{M}^{S}U_{F}^{S}G_{M}G_{F}U | | AUGAUG | |
| | | | | _{F}C_{M}U_{F}C_{M}A_{F}U | | UCACCAA | | | _{M}G_{F}A_{M}U_{F}G_{M}A | | AGAAAC | |
| | | | | _{M}C_{F}A_{M}U_{F}C_{M}A | | TT | | | _{F}U_{M}G_{F}A_{M}G_{F}A | | ATT | |
| | | | | _{F}C_{M}C_{F}A_{M}A_{F}^{S}T | | | | | _{M}A_{F}A_{M}C_{F}A_{M}^{S}T | | | |
| | | | | _{D}^{S}T_{D}-OH-3′ | | | | | _{D}^{S}T_{D}-OH-3′ | | | |
| DT- | 371 | 389 | DTS- | 5′-OH- | 63 | CAUCAUC | 402 | DTS- | 5′-PO4- | 215 | CAUUCG | 578 |
| 000871 | | | 001315 | C_{F}^{S}A_{M}^{S}U_{F}C_{M}A | | ACCAAAC | | 001316 | C_{M}^{S}A_{F}^{S}U_{M}U_{F}C | | UUUGGU | |
| | | | | U_{M}C_{F}A_{M}C_{F}C_{M} | | GAAUGTT | | | _{M}G_{F}U_{M}U_{F}U_{M}G | | GAUGAU | |
| | | | | A_{F}A_{M}A_{F}C_{M}G_{F} | | | | | G_{M}U_{F}G_{M}A_{F}U | | GTT | |
| | | | | A_{M}A_{F}U_{M}G_{F}^{S}T_{D} | | | | | _{M}G_{F}A_{M}U_{F}G_{M}^{S} | | | |
| | | | | ^{S}T_{D}-OH-3′ | | | | | T_{D}^{S}T_{D}-OH-3′ | | | |
| DT- | 383 | 401 | DTS- | 5′-OH- | 64 | ACGAAU | 403 | DTS- | 5′-PO4- | 216 | ACAGAC | |
| 000872 | | | 001317 | A_{F}^{S}C_{M}^{S}G_{F}A_{M}A | | GGCUGC | | 001318 | A_{M}^{S}C_{F}^{S}A_{M}G_{F}A | | UGCAGCC | |
| | | | | _{F}U_{M}G_{F}G_{M}C_{F}U | | AGUCUG | | | _{M}C_{F}U_{M}G_{F}C_{M}A | | AUUCGUT | |
| | | | | _{M}G_{F}C_{M}A_{F}G_{M}U | | UTT | | | _{F}G_{M}C_{F}C_{M}A_{F}U | | | |
| | | | | _{F}C_{M}U_{F}G_{M}U_{F}^{S}T | | | | | _{M}U_{F}C_{M}G_{F}U_{M}^{S}T | | | |
| | | | | _{D}^{S}T_{D}-OH-3′ | | | | | _{D}^{S}T_{D}-OH-3′ | | T | 518 |
| DT- | 407 | 425 | DTS- | 5′-OH- | 65 | CCACCAU | 404 | DTS- | 5′-PO4- | 217 | AUCGAC | 550 |
| 000873 | | | 001319 | C_{F}^{S}C_{M}^{S}A_{F}C_{M}C | | GAUCCU | | 001320 | A_{M}^{S}U_{F}^{S}C_{M}G_{F}A | | AGGAUC | |
| | | | | _{F}A_{M}U_{F}G_{M}A_{F}U | | GUCGAUT | | | _{M}C_{F}A_{M}G_{F}G_{M}A | | AUGGUG | |
| | | | | _{M}C_{F}C_{M}U_{F}G_{M}U | | T | | | _{F}U_{M}C_{F}A_{M}U_{F}G | | GTT | |
| | | | | C_{M}G_{F}A_{M}U_{F}^{S}T | | | | | _{M}G_{F}U_{M}G_{F}G_{M}^{S} | | | |
| | | | | _{D}^{S}T_{D}-OH-3′ | | | | | T_{D}^{S}T_{D}-OH-3′ | | | |
| DT- | 413 | 431 | DTS- | 5′-OH- | 66 | UGAUCC | 405 | DTS- | 5′-PO4- | 218 | AAGAUG | 505 |
| 000874 | | | 001321 | U_{F}^{S}G_{M}^{S}A_{F}U_{M}C | | UGUCGA | | 001322 | A_{M}^{S}A_{F}^{S}G_{M}A_{F}U | | AUCGAC | |
| | | | | _{F}C_{M}U_{F}G_{M}U_{F}C | | UCAUCU | | | _{M}G_{F}A_{M}U_{F}C_{M}G | | AGGAUC | |
| | | | | _{M}G_{F}A_{M}U_{F}C_{M}A | | UTT | | | _{F}A_{M}C_{F}A_{M}G_{F}G | | ATT | |
| | | | | _{F}U_{M}C_{F}U_{M}U_{F}^{S}T | | | | | _{M}A_{F}U_{M}C_{F}A_{M}^{S}T | | | |
| | | | | _{D}^{S}T_{D}-OH-3′ | | | | | _{D}^{S}T_{D}-OH-3′ | | | |
| DT- | 419 | 437 | DTS- | 5′-OH- | 67 | UGUCGA | 406 | DTS- | 5′-PO4- | 219 | AUGCUG | 560 |
| 000875 | | | 001323 | U_{F}^{S}G_{M}^{S}U_{F}C_{M}G | | UCAUCU | | 001324 | A_{M}^{S}U_{F}^{S}G_{M}C_{F}U | | AAGAUG | |
| | | | | _{F}A_{M}U_{F}C_{M}A_{F}U | | UCAGCA | | | _{M}G_{F}A_{M}A_{F}G_{M}A | | AUCGAC | |
| | | | | _{M}C_{F}U_{M}U_{F}C_{M}A | | UTT | | | _{F}U_{M}G_{F}A_{M}U_{F}C | | ATT | |
| | | | | G_{M}C_{F}A_{M}U_{F}^{S}T | | | | | _{M}G_{F}A_{M}C_{F}A_{M}^{S}T | | | |
| | | | | _{D}^{S}T_{D}-OH-3′ | | | | | _{D}^{S}T_{D}-OH-3′ | | | |
| DT- | 449 | 467 | DTS- | 5′-OH- | 68 | UCCUGU | 407 | DTS- | 5′-PO4- | | AGUUGG | 538 |
| 000876 | | | 001325 | U_{F}^{S}C_{M}^{S}C_{F}U_{M}G | | UCUUCU | | 001326 | A_{M}^{S}G_{F}^{S}U_{M}U_{F}G | 220 | CAGAAG | |
| | | | | _{F}U_{M}U_{F}C_{M}U_{F}U | | GCCAACU | | | _{M}G_{F}C_{M}A_{F}G_{M}A | | AACAGG | |
| | | | | _{M}C_{F}U_{M}G_{F}C_{M}C_{F} | | TT | | | _{F}A_{M}G_{F}A_{M}A_{F}C | | ATT | |
| | | | | A_{M}A_{F}C_{M}U_{F}^{S}T_{D} | | | | | _{M}A_{F}G_{M}G_{F}A_{M}^{S} | | | |
| | | | | ^{S}T_{D}-OH-3′ | | | | | T_{D}^{S}T_{D}-OH-3′ | | | |
| DT- | 452 | 470 | DTS- | 5′-OH- | 69 | UGUUCU | 408 | DTS- | 5′-PO4- | 221 | AAGAGU | 503 |
| 000877 | | | 001327 | U_{F}^{S}G_{M}^{S}U_{F}U_{M}C | | UCUGCCA | | 001328 | A_{M}^{S}A_{F}^{S}G_{M}A_{F}G | | UGGCAG | |
| | | | | _{F}U_{M}U_{F}C_{M}U_{F}G | | ACUCUUT | | | _{M}U_{F}U_{M}G_{F}G_{M}C | | AAGAAC | |
| | | | | _{M}C_{F}C_{M}A_{F}A_{M}C_{F} | | T | | | _{F}A_{M}G_{F}A_{M}A_{F}G | | ATT | |
| | | | | U_{M}C_{F}U_{M}U_{F}^{S}T_{D} | | | | | _{M}A_{F}A_{M}C_{F}A_{M}^{S}T | | | |
| | | | | ^{S}T_{D}-OH-3′ | | | | | _{D}^{S}T_{D}-OH-3′ | | | |
| DT- | 460 | 478 | DTS- | 5′-OH- | 70 | UGCCAAC | 409 | DTS- | 5′-PO4- | 222 | UGAGGG | 604 |
| 000878 | | | 001329 | U_{F}^{S}G_{M}C_{F}C_{M}A | | UCUUCAC | | 001330 | U_{M}^{S}G_{F}^{S}A_{M}G_{F}G | | UGAAGA | |
| | | | | _{F}A_{M}C_{F}U_{M}C_{F}U | | CCUCATT | | | _{M}G_{F}U_{M}G_{F}A_{M}A | | GUUGGC | |
| | | | | _{M}U_{F}C_{M}A_{F}C_{M}C_{F} | | | | | _{F}G_{M}A_{F}G_{M}U_{F}U | | ATT | |
| | | | | C_{M}U_{F}C_{M}A_{F}^{S}T_{D} | | | | | _{M}G_{F}G_{M}C_{F}A_{M}^{S}T | | | |
| | | | | ^{S}T_{D}-OH-3′ | | | | | _{D}^{S}T_{D}-OH-3′ | | | |
| DT- | 464 | 482 | DTS- | 5′-OH- | 71 | AACUCU | 410 | DTS- | 5′-PO4- | 223 | UUGGUG | 628 |
| 000879 | | | 001331 | A_{F}^{S}A_{M}^{S}C_{F}U_{M}C | | UCACCCU | | 001332 | U_{M}^{S}U_{F}^{S}G_{M}G_{F}U | | AGGGUG | |
| | | | | _{F}U_{M}U_{F}C_{M}A_{F}C | | CACCAAT | | | _{M}G_{F}A_{M}G_{F}G_{M}G | | AAGAGU | |
| | | | | _{M}C_{F}C_{M}U_{F}C_{M}A_{F} | | T | | | _{F}U_{M}G_{F}A_{M}A_{F}G | | UTT | |
| | | | | C_{M}C_{F}A_{M}A_{F}^{S}T_{D} | | | | | _{M}A_{F}G_{M}U_{F}U_{M}^{S} | | | |
| | | | | ^{S}T_{D}-OH-3′ | | | | | T_{D}^{S}T_{D}-OH-3′ | | | |
| DT- | 486 | 504 | DTS- | 5′-OH- | 72 | GGGCAG | 411 | DTS- | 5′-PO4- | 224 | AGUGAU | 536 |
| 000880 | | | 001333 | G_{F}^{S}G_{M}G_{F}C_{M}A | | GUUUUA | | 001334 | A_{M}^{S}G_{F}^{S}U_{M}G_{F}A | | GUAAAA | |
| | | | | _{F}G_{M}G_{F}U_{M}U_{F}U | | CAUCACU | | | _{M}U_{F}G_{M}U_{F}A_{M}A | | CCUGCCC | |
| | | | | _{M}U_{F}A_{M}C_{F}A_{M}U | | TT | | | _{F}A_{M}A_{F}C_{M}C_{F}U | | TT | |
| | | | | _{F}C_{M}A_{F}C_{M}U_{F}^{S}T | | | | | _{M}G_{F}C_{M}C_{F}C_{M}^{S}T | | | |
| | | | | _{D}^{S}T_{D}-OH-3′ | | | | | _{D}^{S}T_{D}-OH-3′ | | | |
| DT- | 491 | 509 | DTS- | 5′-OH- | 73 | GGUUUU | 412 | DTS- | 5′-PO4- | 225 | AUUCCA | 567 |
| 000881 | | | 001335 | G_{F}^{S}G_{M}^{S}U_{F}U_{M}U | | ACAUCAC | | 001336 | A_{M}^{S}U_{F}^{S}U_{M}C_{F}C | | GUGAUG | |
| | | | | _{F}U_{M}A_{F}C_{M}A_{F}U | | UGGAAU | | | _{M}A_{F}G_{M}U_{F}G_{M}A | | UAAAAC | |
| | | | | _{M}C_{F}A_{M}C_{F}U_{M}G | | TT | | | _{F}U_{M}G_{F}U_{M}A_{F}A | | CTT | |
| | | | | G_{M}A_{F}A_{M}U_{F}^{S}T | | | | | _{M}A_{F}A_{M}C_{F}C_{M}^{S}T | | | |
| | | | | _{D}^{S}T_{D}-OH-3′ | | | | | _{D}^{S}T_{D}-OH-3′ | | | |
| DT- | 494 | 512 | DTS- | 5′-OH- | 74 | UUUACA | 413 | DTS- | 5′-PO4- | 226 | AAGAUU | 506 |
| 000882 | | | 001337 | U_{F}^{S}U_{M}^{S}U_{F}A_{M}C | | UCACUG | | 001338 | A_{M}^{S}A_{F}^{S}G_{M}A_{F}U02 | | CCAGUG | |
| | | | | _{F}A_{M}U_{F}C_{M}A_{F}C | | GAAUCU | | | _{M}U_{F}C_{M}C_{F}A_{M}G | | AUGUAA | |
| | | | | _{M}U_{F}G_{M}G_{F}A_{M}A | | UTT | | | _{F}U_{M}G_{F}A_{M}U_{F}G | | ATT | |
| | | | | _{F}U_{M}C_{F}U_{M}U_{F}^{S}T | | | | | _{M}U_{F}A_{M}A_{F}A_{M}^{S} | | | |
| | | | | _{D}^{S}T_{D}-OH-3′ | | | | | T_{D}^{S}T_{D}-OH-3′ | | | |
| DT- | 501 | 519 | DTS- | 5′-OH- | 75 | CACUGG | 414 | DTS- | 5′-PO4- | 227 | AAUUUG | 515 |
| 000883 | | | 001339 | C_{F}^{S}A_{M}^{S}C_{F}U_{M}G | | AAUCUU | | 001340 | A_{M}^{S}A_{F}^{S}U_{M}U_{F}U | | GAAGAU | |
| | | | | _{F}G_{M}A_{F}A_{M}U_{F}C | | CCAAAU | | | _{M}G_{F}G_{M}A_{F}A_{M}G | | UCCAGU | |
| | | | | _{M}U_{F}U_{M}C_{F}C_{M}A | | UTT | | | _{F}A_{M}U_{F}U_{M}C_{F}C | | GTT | |
| | | | | _{F}A_{M}A_{F}U_{M}U_{F}^{S}T | | | | | _{M}A_{F}G_{M}U_{F}G^{S} | | | |
| | | | | _{D}^{S}T_{D}-OH-3′ | | | | | T_{D}^{S}T_{D}-OH-3′ | | | |
| DT- | 507 | 525 | DTS- | 5′-OH- | 76 | AAUCUU | 415 | DTS- | 5′-PO4- | 228 | AGCAAG | 533 |
| 000884 | | | 001341 | A_{F}^{S}A_{M}^{S}U_{F}C_{M}U | | CCAAAU | | 001342 | A_{M}^{S}G_{F}^{S}C_{M}A_{F}A | | AAUUUG | |
| | | | | _{F}U_{M}C_{F}C_{M}A_{F}A | | UCUUGC | | | _{M}G_{F}A_{M}A_{F}U_{M}U | | GAAGAU | |
| | | | | _{M}A_{F}U_{M}U_{F}C_{M}U | | UTT | | | _{F}U_{M}G_{F}G_{M}A_{F}A | | UTT | |
| | | | | _{F}U_{M}G_{F}C_{M}U_{F}^{S}T | | | | | _{M}G_{F}A_{M}U_{F}U_{M}^{S} | | | |
| | | | | _{D}^{S}T_{D}-OH-3′ | | | | | T_{D}^{S}T_{D}-OH-3′ | | | |
| DT- | 514 | 532 | DTS- | 5′-OH- | 77 | CAAAUU | 416 | DTS- | 5′-PO4- | 229 | ACAGACC | 516 |
| 000885 | | | 001343 | C_{F}^{S}A_{M}^{S}A_{F}A_{M}U | | CUUGCU | | 001344 | A_{M}^{S}C_{F}^{S}A_{M}G_{F}A | | AGCAAG | |
| | | | | _{F}U_{M}C_{F}U_{M}U_{F}G | | GGUCUG | | | _{M}C_{F}C_{M}A_{F}G_{M}C_{F} | | AAUUUG | |
| | | | | _{M}C_{F}U_{M}G_{F}G_{M}U | | UTT | | | A_{M}A_{F}G_{M}A_{F}A_{M} | | TT | |
| | | | | _{F}C_{M}U_{F}G_{M}U_{F}^{S}T | | | | | U_{F}U_{M}U_{F}G_{M}^{S}T_{D} | | | |
| | | | | _{D}^{S}T_{D}-OH-3′ | | | | | ^{S}T_{D}-OH-3′ | | | |
| DT- | 523 | 541 | DTS- | 5′-OH- | 78 | GCUGGU | 417 | DTS- | 5′-PO4- | 230 | UCAUCAC | 600 |
| 000886 | | | 001345 | G_{F}^{S}C_{M}^{S}U_{F}G_{M}G | | CUGUGC | | 001346 | U_{M}^{S}C_{F}^{S}A_{M}U_{F}C | | GCACAG | |
| | | | | _{F}U_{M}C_{F}U_{M}G_{F}U | | GUGAUG | | | _{M}A_{F}C_{M}G_{F}C_{M}A | | ACCAGCT | |
| | | | | _{M}G_{F}C_{M}G_{F}U_{M}G | | ATT | | | C_{M}A_{F}G_{M}A_{F}C | | T | |
| | | | | _{F}A_{M}U_{F}G_{M}A_{F}^{S}T | | | | | _{M}C_{F}A_{M}G_{F}C_{M}^{S}T | | | |
| | | | | _{D}^{S}T_{D}-OH-3′ | | | | | _{D}^{S}T_{D}-OH-3′ | | | |
| DT- | 536 | 554 | DTS- | 5′-OH- | 79 | UGAUGA | 418 | DTS- | 5′-PO4- | 231 | AUGGCC | 561 |
| 000887 | | | 001347 | U_{F}^{S}G_{M}^{S}A_{F}U_{M}G | | GUGCUG | | 001348 | A_{M}^{S}U_{F}^{S}G_{M}G_{F}C | | GCAGCAC | |
| | | | | _{F}A_{M}G_{F}U_{M}G_{F}C | | CGGCCAU | | | _{M}C_{F}G_{M}C_{F}A_{M}G | | UCAUCAT | |
| | | | | _{M}U_{F}G_{M}C_{F}G_{M}G | | TT | | | _{F}C_{M}A_{F}C_{M}U_{F}C_{M} | | T | |
| | | | | _{F}C_{M}C_{F}A_{M}U_{F}^{S}T | | | | | A_{F}U_{M}C_{F}A_{M}^{S}T_{D} | | | |
| | | | | _{D}^{S}T_{D}-OH-3′ | | | | | ^{S}T_{D}-OH-3′ | | | |
| DT- | 539 | 557 | DTS- | 5′-OH- | 80 | UGAGUG | 419 | DTS- | 5′-PO4- | 232 | UAGAUG | 594 |
| 000888 | | | 001349 | U_{F}^{S}G_{M}^{S}A_{F}G_{M}U | | CUGCGGC | | 001350 | U_{M}^{S}A_{F}^{S}G_{M}A_{F}U | | GCCGCAG | |
| | | | | _{F}G_{M}C_{F}U_{M}G_{F}C | | CAUCUAT | | | _{M}G_{F}G_{M}C_{F}C_{M}G | | CACUCAT | |
| | | | | _{M}G_{F}G_{M}C_{F}C_{M}A | | T | | | _{F}C_{M}A_{F}G_{M}C_{F}A | | T | |
| | | | | _{F}U_{M}C_{F}U_{M}A_{F}^{S}T | | | | | _{M}C_{F}U_{M}C_{F}A_{M}^{S}T | | | |
| | | | | _{D}^{S}T_{D}-OH-3′ | | | | | _{D}^{S}T_{D}-OH-3′ | | | |
| DT- | 547 | 565 | DTS- | 5′-OH- | 81 | GCGGCCA | 420 | DTS- | 5′-PO4- | 233 | UCACCGU | 599 |
| 000889 | | | 001351 | G_{F}^{S}C_{M}^{S}G_{F}G_{M}C | | UCUACAC | | 001352 | U_{M}^{S}C_{F}^{S}A_{M}C_{F}C | | GUAGAU | |
| | | | | _{F}C_{M}A_{F}U_{M}C_{F}U | | GGUGATT | | | _{M}G_{F}U_{M}G_{F}U_{M}A | | GGCCGCT | |
| | | | | _{M}A_{F}C_{M}A_{F}C_{M}G | | | | | _{F}G_{M}A_{F}U_{M}G_{F}G | | T | |
| | | | | G_{M}U_{F}G_{M}A_{F}^{S}T | | | | | _{M}C_{F}C_{M}G_{F}C_{M}^{S}T | | | |
| | | | | _{D}^{S}T_{D}-OH-3′ | | | | | _{D}^{S}T_{D}-OH-3′ | | | |
| DT- | 559 | 577 | DTS- | 5′-OH- | 82 | ACGGUG | 421 | DTS- | 5′-PO4- | 234 | ACUCCGG | 528 |
| 000890 | | | 001353 | A_{F}^{S}C_{M}^{S}G_{F}G_{M}U | | AGGCACC | | 001354 | A_{M}C_{F}^{S}U_{M}C_{F}C | | GUGCCUC | |
| | | | | _{F}G_{M}A_{F}G_{M}G_{F}C | | CGGAGUT | | | _{M}G_{F}G_{M}G_{F}U_{M}G | | ACCGUTT | |
| | | | | _{M}A_{F}C_{M}C_{F}C_{M}G_{F} | | T | | | _{F}C_{M}C_{F}U_{M}C_{F}A_{M} | | | |
| | | | | G_{M}A_{F}G_{M}U_{F}^{S}T_{D} | | | | | C_{F}C_{M}G_{F}U_{M}^{S}T_{D} | | | |
| | | | | ^{S}T_{D}-OH-3′ | | | | | ^{S}T_{D}-OH-3′ | | | |
| DT- | 564 | 582 | DTS- | 5′-OH- | 83 | GAGGCA | 422 | DTS- | 5′-PO4- | 235 | AUGCCAC | 559 |
| 000891 | | | 001355 | G_{F}^{S}A_{M}^{S}G_{F}G_{M}C | | CCCGGAG | | 001356 | A_{M}^{S}U_{F}^{S}G_{M}C_{F}C | | UCCGGG | |
| | | | | _{F}A_{M}C_{F}C_{M}C_{F}G_{M} | | UGGCAUT | | | _{M}A_{F}C_{M}U_{F}C_{M}C_{F} | | UGCCUCT | |
| | | | | G_{F}A_{M}G_{F}U_{M}G_{F} | | T | | | G_{M}G_{F}G_{M}U_{F}G_{M} | | T | |
| | | | | G_{M}C_{F}A_{M}U_{F}^{S}T_{D} | | | | | C_{F}C_{M}U_{F}C_{M}^{S}T_{D} | | | |
| | | | | ^{S}T_{D}-OH-3′ | | | | | ^{S}T_{D}-OH-3′ | | | |
| DT- | 569 | 587 | DTS- | 5′-OH- | 84 | ACCCGGA | 423 | DTS- | 5′-PO4- | 236 | UUGAGA | 622 |
| 000892 | | | 001357 | A_{F}^{S}C_{M}^{S}C_{F}C_{M}G | | GUGGCA | | 001358 | U_{M}^{S}U_{F}^{S}G_{M}A_{F}G | | UGCCACU | |
| | | | | _{F}G_{M}A_{F}G_{M}U_{F}G | | UCUCAAT | | | _{M}A_{F}U_{M}G_{F}C_{M}C | | CCGGGUT | |
| | | | | _{M}G_{F}C_{M}A_{F}U_{M}C | | T | | | _{F}A_{M}C_{F}U_{M}C_{F}C_{M} | | T | |
| | | | | _{F}U_{M}C_{F}A_{M}A_{F}^{S}T | | | | | G_{F}G_{M}G_{F}U_{M}T_{D} | | | |
| | | | | _{D}^{S}T_{D}-OH-3′ | | | | | ^{S}T_{D}-OH-3′ | | | |
| DT- | 577 | 595 | DTS- | 5′-OH- | 85 | UGGCAU | 424 | DTS- | 5′-PO4- | 237 | AAUCCG | 513 |
| 000893 | | | 001359 | U_{F}^{S}G_{M}^{S}G_{F}C_{M}A | | CUCAACU | | 001360 | A_{M}^{S}A_{F}^{S}U_{M}C_{F}C | | AGUUGA | |
| | | | | _{F}U_{M}C_{F}U_{M}C_{F}A | | CGGAUUT | | | _{M}G_{F}A_{M}G_{F}U_{M}U | | GAUGCC | |
| | | | | _{M}A_{F}C_{M}U_{F}C_{M}G | | T | | | _{F}G_{M}A_{F}G_{M}A_{F}U | | ATT | |
| | | | | _{F}G_{M}A_{F}U_{M}U_{F}^{S}T | | | | | _{M}G_{F}C_{M}C_{F}A_{M}^{S}T | | | |
| | | | | _{D}^{S}T_{D}-OH-3′ | | | | | _{D}^{S}T_{D}-OH-3′ | | | |
| DT- | 584 | 602 | DTS- | 5′-OH- | 86 | UCAACUC | 425 | DTS- | 5′-PO4- | 238 | UAGGAG | 595 |
| 000894 | | | 001361 | U_{F}^{S}C_{M}^{S}A_{F}A_{M}C | | GGAUUA | | 001362 | U_{M}^{S}A_{F}^{S}G_{M}G_{F}A | | UAAUCC | |
| | | | | _{F}U_{M}C_{F}G_{M}G_{F}A | | CUCCUAT | | | _{M}G_{F}U_{M}A_{F}A_{M}U | | GAGUUG | |
| | | | | _{M}U_{F}U_{M}A_{F}C_{M}U | | T | | | _{F}C_{M}C_{F}G_{M}A_{F}G | | ATT | |
| | | | | _{F}C_{M}C_{F}U_{M}A_{F}^{S}T | | | | | _{M}U_{F}U_{M}G_{F}A_{M}^{S} | | | |
| | | | | _{D}^{S}T_{D}-OH-3′ | | | | | T_{D}^{S}T_{D}-OH-3′ | | | |
| DT- | 590 | 608 | DTS- | 5′-OH- | 87 | CGGAUU | 426 | DTS- | 5′-PO4- | 239 | AAACCG | 493 |
| 000895 | | | 001363 | C_{F}^{S}G_{M}^{S}G_{F}A_{M}U | | ACUCCUA | | 001364 | A_{M}^{S}A_{F}^{S}A_{M}C_{F}C | | UAGGAG | |
| | | | | _{F}U_{M}A_{F}C_{M}U_{F}C | | CGGUUUT | | | _{M}G_{F}U_{M}A_{F}G_{M}G | | UAAUCC | |
| | | | | _{M}C_{F}U_{M}A_{F}C_{M}G | | T | | | _{F}A_{M}G_{F}U_{M}A_{F}A | | GTT | |
| | | | | G_{M}U_{F}U_{M}U_{F}^{S}T | | | | | _{M}U_{F}C_{M}C_{F}G_{M}^{S}T | | | |
| | | | | _{D}^{S}T_{D}-OH-3′ | | | | | _{D}^{S}T_{D}-OH-3′ | | | |
| DT- | 599 | 617 | DTS- | 5′-OH- | 88 | CCUACGG | 427 | DTS- | 5′-PO4- | 240 | AUGUAG | 563 |
| 000896 | | | 001365 | C_{F}^{S}C_{M}^{S}U_{F}A_{M}C | | UUUCGCC | | 001366 | A_{M}^{S}U_{F}^{S}G_{M}U_{F}A | | GCGAAA | |
| | | | | G_{M}G_{F}U_{M}U_{F}U | | UACAUTT | | | _{M}G_{F}G_{M}C_{F}G_{M}A | | CCGUAG | |
| | | | | _{M}C_{F}G_{M}C_{F}C_{M}U_{F} | | | | | _{F}A_{M}A_{F}C_{M}C_{F}G | | GTT | |
| | | | | A_{M}C_{F}A_{M}U_{F}^{S}T_{D} | | | | | _{M}U_{F}A_{M}G_{F}G^{S} | | | |
| | | | | ^{S}T_{D}-OH-3′ | | | | | T_{D}^{S}T_{D}-OH-3′ | | | |
| DT- | 602 | 620 | DTS- | 5′-OH- | 89 | ACGGUU | 428 | DTS- | 5′-PO4- | 241 | AGGAUG | 535 |
| 000897 | | | 001367 | A_{F}^{S}C_{M}^{S}G_{F}G_{M}U | | UCGCCUA | | 001368 | A_{M}^{S}G_{F}^{S}G_{M}A_{F}U | | UAGGCG | |
| | | | | _{F}U_{M}U_{F}C_{M}G_{F}C | | CAUCCUT | | | _{M}G_{F}U_{M}A_{F}G_{M}G | | AAACCG | |
| | | | | _{M}C_{F}U_{M}A_{F}C_{M}A | | T | | | _{F}C_{M}G_{F}A_{M}A_{F}A | | UTT | |
| | | | | _{F}U_{M}C_{F}C_{M}U_{F}^{S}T | | | | | _{M}C_{F}C_{M}G_{F}U_{M}^{S}T | | | |
| | | | | _{D}^{S}T_{D}-OH-3′ | | | | | _{D}^{S}T_{D}-OH-3′ | | | |
| DT- | 617 | 635 | DTS- | 5′-OH- | 90 | UCCUGGC | 429 | DTS- | 5′-PO4- | 242 | AAGGCC | 508 |
| 000898 | | | 001369 | U_{F}^{S}C_{M}^{S}C_{F}U_{M}G | | CUGGGU | | 001370 | A_{M}^{S}A_{F}^{S}G_{M}G_{F}C | | ACCCAGG | |
| | | | | _{F}G_{M}C_{F}C_{M}U_{F}G | | GGCCUUT | | | _{M}C_{F}A_{M}C_{F}C_{M}C_{F} | | CCAGGAT | |
| | | | | _{M}G_{F}G_{M}U_{F}G_{M}G | | T | | | A_{M}G_{F}G_{M}C_{F}C_{M} | | T | |
| | | | | _{F}C_{M}C_{F}U_{M}U_{F}^{S}T | | | | | A_{F}G_{M}G_{F}A_{M}^{S}T_{D} | | | |
| | | | | _{D}^{S}T_{D}-OH-3′ | | | | | ^{S}T_{D}-OH-3′ | | | |
| DT- | 644 | 662 | DTS- | 5′-OH- | 91 | CCCUUCU | 430 | DTS- | 5′-PO4- | 243 | AUGACA | 553 |
| 000899 | | | 001371 | C_{F}^{S}C_{M}^{S}C_{F}U_{M}U | | CAGCGG | | 001372 | A_{M}^{S}U_{F}^{S}G_{M}A_{F}C | | CCGCUGA | |
| | | | | _{F}C_{M}U_{F}C_{M}A_{F}G | | UGUCAUT | | | _{M}A_{F}C_{M}C_{F}G_{M}C_{F} | | GAAGGG | |
| | | | | _{M}C_{F}G_{M}G_{F}U_{M}G | | T | | | U_{M}G_{F}A_{M}G_{F}A_{M} | | TT | |
| | | | | _{F}U_{M}C_{F}A_{M}U_{F}^{S}T | | | | | A_{F}G_{M}G_{F}G_{M}^{S}T_{D} | | | |
| | | | | _{D}^{S}T_{D}-OH-3′ | | | | | ^{S}T_{D}-OH-3′ | | | |
| DT- | 653 | 671 | DTS- | 5′-OH- | 92 | GCGGUG | 431 | DTS- | 5′-PO4- | 244 | AUCACA | 545 |
| 000900 | | | 001373 | G_{F}^{S}C_{M}^{S}G_{F}G_{M}U | | UCAUCU | | 001374 | A_{M}^{S}U_{F}^{S}C_{M}A_{F}C | | UAGAUG | |
| | | | | _{F}G_{M}U_{F}C_{M}A_{F}U | | AUGUGA | | | _{M}A_{F}U_{M}A_{F}G_{M}A | | ACACCGC | |
| | | | | _{M}C_{F}U_{M}A_{F}U_{M}G | | UTT | | | _{F}U_{M}G_{F}A_{M}C_{F}A | | TT | |
| | | | | _{F}U_{M}G_{F}A_{M}U_{F}^{S}T | | | | | _{M}C_{F}C_{M}G_{F}C_{M}^{S}T | | | |
| | | | | _{D}^{S}T_{D}-OH-3′ | | | | | _{D}^{S}T_{D}-OH-3′ | | | |
| DT- | 656 | 674 | DTS- | 5′-OH- | 93 | GUGUCA | 432 | DTS- | 5′-PO4- | 245 | AAGAUC | 504 |
| 000901 | | | 001375 | G_{F}^{S}U_{M}G_{F}U_{M}C | | UCUAUG | | 001376 | A_{M}^{S}A_{F}^{S}G_{M}A_{F}U | | ACAUAG | |
| | | | | _{F}A_{M}U_{F}C_{M}U_{F}A | | UGAUCU | | | _{M}C_{F}A_{M}C_{F}A_{M}U | | AUGACA | |
| | | | | _{M}U_{F}G_{M}U_{F}G_{M}A | | UTT | | | _{F}A_{M}G_{F}A_{M}U_{F}G | | CTT | |
| | | | | _{F}U_{M}C_{F}U_{M}U_{F}^{S}T | | | | | _{M}A_{F}C_{M}A_{F}C_{M}^{S}T | | | |
| | | | | _{D}^{S}T_{D}-OH-3′ | | | | | _{D}^{S}T_{D}-OH-3′ | | | |
| DT- | 663 | 681 | DTS- | 5′-OH- | 94 | CUAUGU | 433 | DTS- | 5′-PO4- | 246 | UUUCCGC | 631 |
| 000902 | | | 001377 | C_{F}^{S}U_{M}^{S}A_{F}U_{M}G | | GAUCUU | | 001378 | U_{M}^{S}U_{F}^{S}U_{M}C_{F}C | | AAGAUC | |
| | | | | U_{M}G_{F}A_{M}U_{F}C | | GCGGAA | | | _{M}G_{F}C_{M}A_{F}A_{M}G | | ACAUAGT | |
| | | | | _{M}U_{F}U_{M}G_{F}C_{M}G | | ATT | | | _{F}A_{M}U_{F}C_{M}A_{F}C | | T | |
| | | | | _{F}G_{M}A_{F}A_{M}A_{F}^{S}T | | | | | _{M}A_{F}U_{M}A_{F}G^{S} | | | |
| | | | | _{D}^{S}T_{D}-OH-3′ | | | | | T_{D}^{S}T_{D}-OH-3′ | | | |
| DT- | | | DTS- | 5′-OH- | 95 | AUCUUG | 434 | DTS- | 5′-PO4- | 247 | AUUCGC | 568 |
| 000903 | | | 001379 | A_{F}^{S}U_{M}^{S}C_{F}U_{M}U | | CGGAAA | | 001380 | A_{M}^{S}U_{F}^{S}U_{M}C_{F}G | | GUUUCC | |
| | | | | G_{M}C_{F}G_{M}G_{F}A | | CGCGAA | | | _{M}C_{F}G_{M}U_{F}U_{M}U | | GCAAGA | |
| | | | | _{M}A_{F}A_{M}C_{F}G_{M}C | | UTT | | | _{F}C_{M}C_{F}G_{M}C_{F}A_{M} | | UTT | |
| | 670 | 688 | | _{F}G_{M}A_{F}A_{M}U_{F}^{S}T | | | | | A_{F}G_{M}A_{F}U_{M}^{S}T_{D} | | | |
| | | | | _{D}^{S}T_{D}-OH-3′ | | | | | ^{S}T_{D}-OH-3′ | | | |
| DT- | 711 | 729 | DTS- | 5′-OH- | 96 | GAGGCU | 435 | DTS- | 5′-PO4- | 248 | UAUGUA | 598 |
| 000904 | | | 001381 | G_{F}^{S}A_{M}^{S}G_{F}G_{M}C | | CUGAGC | | 001382 | U_{M}^{S}A_{F}^{S}U_{M}G_{F}U | | CGCUCAG | |
| | | | | U_{M}C_{F}U_{M}G_{F}A | | GUACAU | | | _{M}A_{F}C_{M}G_{F}C_{M}U | | AGCCUCT | |
| | | | | _{M}G_{F}C_{M}G_{F}U_{M}A | | ATT | | | _{F}C_{M}A_{F}G_{M}A_{F}G | | T | |
| | | | | _{F}C_{M}A_{F}U_{M}A_{F}^{S}T | | | | | _{M}C_{F}C_{M}U_{F}C_{M}^{S}T | | | |
| | | | | _{D}^{S}T_{D}-OH-3′ | | | | | _{D}^{S}T_{D}-OH-3′ | | | |
| DT- | 737 | 755 | DTS- | 5′-OH- | 97 | GAGGAA | 436 | DTS- | 5′-PO4- | 249 | UUUCUG | 635 |
| 000905 | | | 001383 | G_{F}^{S}A_{M}^{S}G_{F}G_{M}A | | GGGAAA | | 001384 | U_{M}^{S}U_{F}^{S}U_{M}C_{F}U | | UUUUCCC | |
| | | | | _{F}A_{M}G_{F}G_{M}G_{F}A | | ACAGAA | | | _{M}G_{F}U_{M}U_{F}U_{M}U | | UUCCUCT | |
| | | | | _{M}A_{F}A_{M}A_{F}C_{M}A | | ATT | | | C_{M}C_{F}C_{M}U_{F}U_{M} | | T | |
| | | | | G_{M}A_{F}A_{M}A_{F}^{S}T | | | | | C_{F}C_{M}U_{F}C_{M}^{S}T_{D} | | | |
| | | | | _{D}T_{D}-OH-3′ | | | | | ^{S}T_{D}-OH-3′ | | | |
| DT- | 779 | 797 | DTS- | 5′-OH- | 98 | CCCAAAA | 437 | DTS- | 5′-PO4- | 250 | UUGAGU | 623 |
| 000906 | | | 001385 | C_{F}^{S}C_{M}^{S}C_{F}A_{M}A | | UCCCAAA | | 001386 | U_{M}^{S}U_{F}^{S}G_{M}A_{F}G | | UUGGGA | |
| | | | | _{F}A_{M}A_{F}U_{M}C_{F}C | | CUCAATT | | | _{M}U_{F}U_{M}U_{F}G_{M}G | | UUUUGG | |
| | | | | _{M}C_{F}A_{M}A_{F}A_{M}C | | | | | G_{M}A_{F}U_{M}U_{F}U | | GTT | |
| | | | | _{F}U_{M}C_{F}A_{M}A_{F}^{S}T | | | | | _{M}U_{F}G_{M}G_{F}G_{M}^{S} | | | |
| | | | | _{D}^{S}T_{D}-OH-3′ | | | | | T_{D}^{S}T_{D}-OH-3′ | | | |
| DT- | 785 | 803 | DTS- | 5′-OH- | 99 | AUCCCAA | 438 | DTS- | 5′-PO4- | 251 | UUUGGU | 641 |
| | | | 001387 | A_{F}^{S}U_{M}^{S}C_{F}C_{M}C | | ACUCAA | | 001388 | U_{M}^{S}U_{F}^{S}U_{M}G_{F}G | | UUGAGU | |
| | | | | _{F}A_{M}A_{F}A_{M}C_{F}U | | ACCAAAT | | | _{M}U_{F}U_{M}U_{F}G_{M}A | | UUGGGA | |
| | | | | _{M}C_{F}A_{M}A_{F}A_{M}C | | T | | | _{F}G_{M}U_{F}U_{M}U_{F}G | | UTT | |
| | | | | _{F}C_{M}A_{F}A_{M}A_{F}^{S}T | | | | | _{M}G_{F}G_{M}A_{F}U_{M}^{S} | | | |
| 000907 | | | | _{D}^{S}T_{D}-OH-3′ | | | | | T_{D}^{S}T_{D}-OH-3′ | | | |
| DT- | 833 | 851 | DTS- | 5′-OH- | 100 | GCUGUU | 439 | DTS- | 5′-PO4- | 252 | UACAUC | 591 |
| 000908 | | | 001389 | G_{F}^{S}C_{M}^{S}U_{F}G_{M}U | | GAUUGA | | 001390 | U_{M}^{S}A_{F}^{S}C_{M}A_{F}U | | UUCAAU | |
| | | | | _{F}U_{M}G_{F}A_{M}U_{F}U | | AGAUGU | | | _{M}C_{F}U_{M}U_{F}C_{M}A | | CAACAGC | |
| | | | | _{M}G_{F}A_{M}A_{F}G_{M}A | | ATT | | | _{F}A_{M}U_{F}C_{M}A_{F}A | | TT | |
| | | | | _{F}U_{M}G_{F}U_{M}A_{F}^{S}T | | | | | _{M}C_{F}A_{M}G_{F}C_{M}^{S}T | | | |
| | | | | _{D}^{S}T_{D}-OH-3′ | | | | | _{D}^{S}T_{D}-OH-3′ | | | |
| DT- | 834 | 852 | DTS- | 5′-OH- | 101 | CUGUUG | 440 | DTS- | 5′-PO4- | 253 | AUACAU | 541 |
| | | | 001391 | C_{F}^{S}U_{M}^{S}G_{F}U_{M}U | | AUUGAA | | 001392 | A_{M}^{S}U_{F}^{S}A_{M}C_{F}A | | CUUCAA | |
| | | | | G_{M}A_{F}U_{M}U_{F}G | | GAUGUA | | | _{M}U_{F}C_{M}U_{F}U_{M}C | | UCAACA | |
| | | | | _{M}A_{F}A_{M}G_{F}A_{M}U | | UTT | | | _{F}A_{M}A_{F}U_{M}C_{F}A | | GTT | |
| | | | | G_{M}U_{F}A_{M}U_{F}^{S}T | | | | | _{M}A_{F}C_{M}A_{F}G_{M}^{S}T | | | |
| 000909 | | | | _{D}^{S}T_{D}-OH-3′ | | | | | _{D}^{S}T_{D}-OH-3′ | | | |
| DT- | 861 | 879 | DTS- | 5′-OH- | 102 | UCCGGU | 441 | DTS- | 5′-PO4- | 254 | AUAGGU | 544 |
| 000910 | | | 001393 | U_{F}^{S}C_{M}C_{F}G_{M}G | | UUAUAA | | 001394 | A_{M}^{S}U_{F}^{S}A_{M}G_{F}G | | UUUAUA | |
| | | | | _{F}U_{M}U_{F}U_{M}A_{F}U | | AACCUA | | | _{M}U_{F}U_{M}U_{F}U_{M}A | | AACCGG | |
| | | | | _{M}A_{F}A_{M}A_{F}A_{M}C | | UTT | | | _{F}U_{M}A_{F}A_{M}A_{F}C | | ATT | |
| | | | | _{F}C_{M}U_{F}A_{M}U_{F}^{S}T | | | | | _{M}C_{F}G_{M}G_{F}A_{M}^{S}T | | | |
| | | | | _{D}^{S}T_{D}-OH-3′ | | | | | _{D}^{S}T_{D}-OH-3′ | | | |
| DT- | 865 | 883 | DTS- | 5′-OH- | 103 | GUUUAU | 442 | DTS- | 5′-PO4- | 255 | AUAAAU | 539 |
| 000911 | | | 001395 | G_{F}^{S}U_{M}^{S}U_{F}U_{M}A | | AAAACC | | 001396 | A_{M}^{S}U_{F}^{S}A_{M}A_{F}A | | AGGUUU | |
| | | | | _{F}U_{M}A_{F}A_{M}A_{F}A | | UAUUUA | | | _{M}U_{F}A_{M}G_{F}G_{M}U | | UAUAAA | |
| | | | | _{M}C_{F}C_{M}U_{F}A_{M}U | | UTT | | | _{F}U_{M}U_{F}U_{M}A_{F}U | | CTT | |
| | | | | _{F}U_{M}U_{F}A_{M}U_{F}^{S}T | | | | | _{M}A_{F}A_{M}A_{F}C_{M}^{S}T | | | |
| | | | | _{D}^{S}T_{D}-OH-3′ | | | | | _{D}^{S}T_{D}-OH-3′ | | | |
| DT- | 904 | 922 | DTS- | 5′-OH- | 104 | ACAUAG | 443 | DTS- | 5′-PO4- | 256 | AAAGCA | 494 |
| 000912 | | | 001397 | A_{F}C_{M}^{S}A_{F}U_{M}A | | UAUUGU | | 001398 | A_{M}^{S}A_{F}^{S}A_{M}G_{F}C | | AACAAU | |
| | | | | _{F}G_{M}U_{F}A_{M}U_{F}U | | UUGCUU | | | _{M}A_{F}A_{M}A_{F}C_{M}A | | ACUAUG | |
| | | | | _{M}G_{F}U_{M}U_{F}U_{M}G | | UTT | | | _{F}A_{M}U_{F}A_{M}C_{F}U | | UTT | |
| | | | | _{F}C_{M}U_{F}U_{M}U_{F}^{S}T | | | | | _{M}A_{F}U_{M}G_{F}U_{M}^{S} | | | |
| | | | | _{D}^{S}T_{D}-OH-3′ | | | | | T_{D}^{S}T_{D}-OH-3′ | | | |
| DT- | 929 | 947 | DTS- | 5′-OH- | 105 | UGACCA | 444 | DTS- | 5′-PO4- | 257 | AACACG | 497 |
| 000913 | | | 001399 | U_{F}^{S}G_{M}^{S}A_{F}C_{M}C | | UCAGCCU | | 001400 | A_{M}^{S}A_{F}^{S}C_{M}A_{F}C | | AGGCUG | |
| | | | | _{F}A_{M}U_{F}C_{M}A_{F}G | | CGUGUUT | | | _{M}G_{F}A_{M}G_{F}G_{M}C | | AUGGUC | |
| | | | | _{M}C_{F}C_{M}U_{F}C_{M}G_{F} | | T | | | _{F}U_{M}G_{F}A_{M}U_{F}G | | ATT | |
| | | | | U_{M}G_{F}U_{M}U_{F}^{S}T_{D} | | | | | _{M}G_{F}U_{M}C_{F}A_{M}^{S}T | | | |
| | | | | ^{S}T_{D}-OH-3′ | | | | | _{D}^{S}T_{D}-OH-3′ | | | |
| DT- | 950 | 968 | DTS- | 5′-OH- | 106 | GCCUUA | 445 | DTS- | 5′-PO4- | 258 | UUAGCU | 609 |
| 000914 | | | 001401 | G_{F}^{S}C_{M}^{S}C_{F}U_{M}U | | AAGAAG | | 001402 | U_{M}^{S}U_{F}^{S}A_{M}G_{F}C | | ACUUCU | |
| | | | | _{F}A_{M}A_{F}A_{M}G_{F}A | | UAGCUA | | | _{M}U_{F}A_{M}C_{F}U_{M}U | | UUAAGG | |
| | | | | _{M}A_{F}G_{M}U_{F}A_{M}G | | ATT | | | _{F}C_{M}U_{F}U_{M}U_{F}A | | CTT | |
| | | | | C_{M}U_{F}A_{M}A_{F}^{S}T | | | | | _{M}A_{F}G_{M}G_{F}C_{M}^{S}T | | | |
| | | | | _{D}^{S}T_{D}-OH-3′ | | | | | _{D}^{S}T_{D}-OH-3′ | | | |
| DT- | 958 | 976 | DTS- | 5′-OH- | 107 | GAAGUA | 446 | DTS- | 5′-PO4- | 259 | AAAGUU | 495 |
| 000915 | | | 001403 | G_{F}^{S}A_{M}^{S}A_{F}G_{M}U | | GCUAAG | | 001404 | A_{M}^{S}A_{F}^{S}A_{M}G_{F}U | | CCUUAGC | |
| | | | | _{F}A_{M}G_{F}C_{M}U_{F}A | | GAACUU | | | _{M}U_{F}C_{M}C_{F}U_{M}U | | UACUUCT | |
| | | | | _{M}A_{F}G_{M}G_{F}A_{M}A | | UTT | | | _{F}A_{M}G_{F}C_{M}U_{F}A | | T | |
| | | | | _{F}C_{M}U_{F}U_{M}U_{F}^{S}T | | | | | _{M}C_{F}U_{M}U_{F}C_{M}^{S}T | | | |
| | | | | _{D}^{S}T_{D}-OH-3′ | | | | | _{D}^{S}T_{D}-OH-3′ | | | |
| DT- | 967 | 985 | DTS- | 5′-OH- | 108 | AAGGAA | 447 | DTS- | 5′-PO4- | 260 | UUAGGA | 610 |
| 000916 | | | 001405 | A_{F}^{S}A_{M}G_{F}G_{M}A | | CUUUAC | | 001406 | U_{M}^{S}U_{F}^{S}A_{M}G_{F}G | | UGUAAA | |
| | | | | _{F}A_{M}C_{F}U_{M}U_{F}U | | AUCCUA | | | _{M}A_{F}U_{M}G_{F}U_{M}A | | GUUCCU | |
| | | | | _{M}A_{F}C_{M}A_{F}U_{M}C | | ATT | | | _{F}A_{M}A_{F}G_{M}U_{F}U | | UTT | |
| | | | | _{F}C_{M}U_{F}A_{M}A_{F}^{S}T | | | | | _{M}C_{F}C_{M}U_{F}U_{M}^{S}T | | | |
| | | | | _{D}^{S}T_{D}-OH-3′ | | | | | _{D}^{S}T_{D}-OH-3′ | | | |
| DT- | 975 | 993 | DTS- | 5′-OH- | 109 | UUACAU | 448 | DTS- | 5′-PO4- | 261 | UUAUAC | 612 |
| 000917 | | | 001407 | U_{F}^{S}U_{M}^{S}A_{F}C_{M}A | | CCUAACA | | 001408 | U_{M}^{S}U_{F}^{S}A_{M}U_{F}A | | UGUUAG | |
| | | | | _{F}U_{M}C_{F}C_{M}U_{F}A | | GUAUAA | | | _{M}C_{F}U_{M}G_{F}U_{M}U | | GAUGUA | |
| | | | | _{M}A_{F}C_{M}A_{F}G_{M}U | | TT | | | _{F}A_{M}G_{F}G_{M}A_{F}U | | ATT | |
| | | | | _{F}A_{M}U_{F}A_{M}A_{F}^{S}T | | | | | _{M}G_{F}U_{M}A_{F}A_{M}^{S} | | | |
| | | | | _{D}^{S}T_{D}-OH-3′ | | | | | T_{D}^{S}T_{D}-OH-3′ | | | |
| DT- | 976 | 994 | DTS- | 5′-OH- | 110 | UACAUCC | 449 | DTS- | 5′-PO4- | 262 | AUUAUA | 566 |
| 000918 | | | 001409 | U_{F}^{S}A_{M}^{S}C_{F}A_{M}U | | UAACAG | | 001410 | A_{M}^{S}U_{F}^{S}U_{M}A_{F}U | | CUGUUA | |
| | | | | C_{M}C_{F}U_{M}A_{F}A | | UAUAAU | | | _{M}A_{F}C_{M}U_{F}G_{M}U | | GGAUGU | |
| | | | | _{M}C_{F}A_{M}G_{F}U_{M}A | | TT | | | _{F}U_{M}A_{F}G_{M}G_{F}A | | ATT | |
| | | | | _{F}U_{M}A_{F}A_{M}U_{F}^{S}T | | | | | _{M}U_{F}G_{M}U_{F}A_{M}^{S} | | | |
| | | | | _{D}^{S}T_{D}-OH-3′ | | | | | T_{D}^{S}T_{D}-OH-3′ | | | |
| DT | 1039 | 1057 | DTS- | 5′-OH- | 111 | UUACCCA | 450 | DTS- | 5′-PO4- | 263 | UUAUCU | 613 |
| 000919 | | | 001411 | U_{F}^{S}U_{M}^{S}A_{F}C_{M}C | | GAAAUA | | 001412 | U_{M}^{S}U_{F}^{S}A_{M}U_{F}C | | UAUUUC | |
| | | | | C_{M}A_{F}G_{M}A_{F}A | | AGAUAA | | | _{M}U_{F}U_{M}A_{F}U_{M}U | | UGGGUA | |
| | | | | _{M}A_{F}U_{M}A_{F}A_{M}G | | TT | | | _{F}U_{M}C_{F}U_{M}G_{F}G | | ATT | |
| | | | | _{F}A_{M}U_{F}A_{M}A_{F}^{S}T | | | | | _{M}G_{F}U_{M}A_{F}A_{M}^{S} | | | |
| | | | | _{D}^{S}T_{D}-OH-3′ | | | | | T_{D}^{S}T_{D}-OH-3′ | | | |
| DT- | 1069 | 1087 | DTS- | 5′-OH- | 112 | CCCUUCC | 451 | DTS- | 5′-PO4- | 264 | UUCAGA | 614 |
| 000920 | | | 001413 | C_{F}^{S}C_{M}^{S}C_{F}U_{M}U | | CUUUCA | | 001414 | U_{M}^{S}U_{F}^{S}C_{M}A_{F}G | | UGAAAG | |
| | | | | _{F}C_{M}C_{F}C_{M}U_{F}U_{M} | | UCUGAAT | | | _{M}A_{F}U_{M}G_{F}A_{M}A | | GGAAGG | |
| | | | | U_{F}C_{M}A_{F}U_{M}C_{F} | | T | | | _{F}A_{M}G_{F}G_{M}G_{F}A | | GTT | |
| | | | | U_{M}G_{F}A_{M}A_{F}^{S}T_{D} | | | | | _{M}A_{F}G_{M}G_{F}G_{M}′^{S} | | | |
| | | | | ^{S}T_{D}-OH-3′ | | | | | T_{D}^{S}T_{D}-OH-3′ | | | |
| DT- | 1180 | 1198 | DTS- | 5′-OH- | 113 | CCAGUGC | 452 | DTS- | 5′-PO4- | 265 | UUUCUG | 634 |
| 000921 | | | 001415 | CE^{S}C_{M}^{S}A_{F}G_{M}U | | AUCCAAC | | 001416 | U_{M}^{S}U_{F}^{S}U_{M}C_{F}U | | UUGGAU | |
| | | | | _{F}G_{M}C_{F}A_{M}U_{F}C | | AGAAATT | | | _{M}G_{F}U_{M}U_{F}G_{M}G | | GCACUG | |
| | | | | _{M}C_{F}A_{M}A_{F}C_{M}A | | | | | _{F}A_{M}U_{F}G_{M}C_{F}A | | GTT | |
| | | | | _{F}G_{M}A_{F}A_{M}A_{F}^{S}T | | | | | _{M}C_{F}U_{M}G_{F}G_{M}^{S}T | | | |
| | | | | _{D}^{S}T_{D}-OH-3′ | | | | | _{D}^{S}T_{D}-OH-3′ | | | |
| DT- | 1213 | 1231 | DTS- | 5′-OH- | 114 | ACCUCUG | 453 | DTS- | 5′-PO4- | 266 | UAAAGC | 586 |
| 000922 | | | 001417 | A_{F}^{S}C_{M}^{S}C_{F}U_{M}C | | UGUGAA | | 001418 | U_{M}^{S}A_{F}^{S}A_{M}A_{F}G | | UUCACAC | |
| | | | | _{F}U_{M}G_{F}U_{M}G_{F}U | | GCUUUAT | | | _{M}C_{F}U_{M}U_{F}C_{M}A | | AGAGGU | |
| | | | | _{M}G_{F}A_{M}A_{F}G_{M}C | | T | | | _{F}C_{M}A_{F}C_{M}A_{F}G | | TT | |
| | | | | _{F}U_{M}U_{F}U_{M}A_{F}^{S}T | | | | | _{M}A_{F}G_{M}G_{F}U_{M}^{S} | | | |
| | | | | _{D}^{S}T_{D}-OH-3′ | | | | | T_{D}^{S}T_{D}-OH-3′ | | | |
| DT- | 1695 | 1713 | DTS- | 5′-OH- | 115 | CCACCAA | 454 | DTS- | 5′-PO4- | 267 | AUACAU | 540 |
| 000923 | | | 001419 | C_{F}^{S}C_{M}^{S}A_{F}C_{M}C | | CUGUAG | | 001420 | A_{M}^{S}U_{F}^{S}A_{M}C_{F}A | | CUACAG | |
| | | | | _{F}A_{M}A_{F}C_{M}U_{F}G | | AUGUAU | | | _{M}U_{F}C_{M}U_{F}A_{M}C | | UUGGUG | |
| | | | | _{M}U_{F}A_{M}G_{F}A_{M}U | | TT | | | _{F}A_{M}G_{F}U_{M}U_{F}G | | GTT | |
| | | | | _{F}G_{M}U_{F}A_{M}U_{F}^{S}T | | | | | _{M}G_{F}U_{M}G_{F}G_{M}^{S} | | | |
| | | | | _{D}^{S}T_{D}-OH-3′ | | | | | T_{D}^{S}T_{D}-OH-3′ | | | |
| DT- | 1727 | 1745 | DTS- | 5′-OH- | 116 | CUGAUG | 455 | DTS- | 5′-PO4- | 268 | UCUGGA | 602 |
| 000924 | | | 001421 | C_{F}^{S}U_{M}^{S}G_{F}A_{M}U | | CUAAGA | | 001422 | U_{M}^{S}C_{F}^{S}U_{M}G_{F}G | | GUCUUA | |
| | | | | G_{M}C_{F}U_{M}A_{F}A | | CUCCAGA | | | _{M}A_{F}G_{M}U_{F}C_{M}U | | GCAUCA | |
| | | | | _{M}G_{F}A_{M}C_{F}U_{M}C | | TT | | | _{F}U_{M}A_{F}G_{M}C_{F}A | | GTT | |
| | | | | _{F}C_{M}A_{F}G_{M}A_{F}^{S}T | | | | | _{M}U_{F}C_{M}A_{F}G_{M}^{S}T | | | |
| | | | | _{D}^{S}T_{D}-OH-3′ | | | | | _{D}^{S}T_{D}-OH-3′ | | | |
| DT- | 1757 | 1775 | DTS- | 5′-OH- | 117 | UGCUUU | 456 | DTS- | 5′-PO4- | 269 | AAUCAG | 512 |
| 000925 | | | 001423 | U_{F}^{S}G_{M}^{S}C_{F}U_{M}U | | GCAUUU | | 001424 | A_{M}^{S}A_{F}^{S}U_{M}C_{F}A | | AAAAUG | |
| | | | | _{F}U_{M}G_{F}C_{M}A_{F}U | | UCUGAU | | | _{M}G_{F}A_{M}A_{F}A_{M}A | | CAAAGC | |
| | | | | _{M}U_{F}U_{M}U_{F}C_{M}U | | UTT | | | _{F}U_{M}G_{F}C_{M}A_{F}A | | ATT | |
| | | | | _{F}G_{M}A_{F}U_{M}U_{F}^{S}T | | | | | _{M}A_{F}G_{M}C_{F}A_{M}^{S}T | | | |
| | | | | _{D}^{S}T_{D}-OH-3′ | | | | | _{D}^{S}T_{D}-OH-3′ | | | |
| DT- | 1779 | 1797 | DTS- | 5′-OH- | 118 | UACCAAC | 457 | DTS- | 5′-PO4- | 270 | UUAGUC | 611 |
| 000926 | | | 001425 | U_{F}^{S}A_{M}^{S}C_{F}C_{M}A | | UGUGUG | | 001426 | U_{M}^{S}U_{F}^{S}A_{M}G_{F}U | | CACACAG | |
| | | | | _{F}A_{M}C_{F}U_{M}G_{F}U | | GACUAAT | | | _{M}C_{F}C_{M}A_{F}C_{M}A_{F} | | UUGGUA | |
| | | | | _{M}G_{F}U_{M}G_{F}G_{M}A | | T | | | C_{M}A_{F}G_{M}U_{F}U_{M} | | TT | |
| | | | | _{F}C_{M}U_{F}A_{M}A_{F}^{S}T | | | | | G_{F}G_{M}U_{F}A_{M}^{S}T_{D} | | | |
| | | | | _{D}^{S}T_{D}-OH-3′ | | | | | ^{S}T_{D}-OH-3′ | | | |
| DT- | 1782 | 1800 | DTS- | 5′-OH- | 119 | CAACUG | 458 | DTS- | 5′-PO4- | 271 | AUCUUA | 552 |
| 000927 | | | 001427 | C_{F}^{S}A_{M}^{S}A_{F}C_{M}U | | UGUGGA | | 001428 | A_{M}^{S}U_{F}^{S}C_{M}U_{F}U | | GUCCACA | |
| | | | | _{F}G_{M}U_{F}G_{M}U_{F}G | | CUAAGA | | | _{M}A_{F}G_{M}U_{F}C_{M}C | | CAGUUGT | |
| | | | | _{M}G_{F}A_{M}C_{F}U_{M}A | | UTT | | | _{F}A_{M}C_{F}A_{M}C_{F}A | | T | |
| | | | | _{F}A_{M}G_{F}A_{M}U_{F}^{S}T | | | | | _{M}G_{F}U_{M}U_{F}G_{M}^{S} | | | |
| | | | | _{D}^{S}T_{D}-OH-3′ | | | | | T_{D}^{S}T_{D}-OH-3′ | | | |
| DT- | 1786 | 1804 | DTS- | 5′-OH- | 120 | UGUGUG | 459 | DTS- | 5′-PO4- | 272 | AUGCAU | 558 |
| 000928 | | | 001429 | U_{F}^{S}G_{M}^{S}U_{F}G_{M}U | | GACUAA | | 001430 | A_{M}^{S}U_{F}^{S}G_{M}C_{F}A | | CUUAGU | |
| | | | | _{F}G_{M}G_{F}A_{M}C_{F}U | | GAUGCA | | | _{M}U_{F}C_{M}U_{F}U_{M}A | | CCACACA | |
| | | | | _{M}A_{F}A_{M}G_{F}A_{M}U | | UTT | | | _{F}G_{M}U_{F}C_{M}C_{F}A | | TT | |
| | | | | _{F}G_{M}C_{F}A_{M}U_{F}^{S}T | | | | | _{M}C_{F}A_{M}C_{F}A_{M}^{S}T | | | |
| | | | | _{D}^{S}T_{D}-OH-3′ | | | | | _{D}^{S}T_{D}-OH-3′ | | | |
| DT- | 7 | 25 | DTS- | 5′-OH- | 121 | AGGGAG | 460 | DTS- | 5′-PO4- | 273 | UUCCCUG | 616 |
| 001010 | | | 001563 | A_{F}^{S}G_{M}^{S}G_{F}G_{M}A | | CACCACC | | 001564 | U_{M}^{S}U_{F}^{S}C_{M}C_{F}C | | GUGGUG | |
| | | | | G_{M}C_{F}A_{M}C_{F}C_{M} | | AGGGAA | | | _{M}U_{F}G_{M}G_{F}U_{M}G | | CUCCCUT | |
| | | | | A_{F}C_{M}C_{F}A_{M}G_{F} | | TT | | | _{F}G_{M}U_{F}G_{M}C_{F}U | | T | |
| | | | | G_{M}G_{F}A_{M}A_{F}^{S}T_{D} | | | | | _{M}C_{F}C_{M}C_{F}U_{M}^{S}T | | | |
| | | | | ^{S}T_{D}-OH-3′ | | | | | _{D}^{S}T_{D}-OH-3′ | | | |
| DT- | 10 | 28 | DTS- | 5′-OH- | 122 | GAGCACC | 461 | DTS- | 5′-PO4- | 274 | AUGUUC | 565 |
| 001011 | | | 001565 | G_{F}^{S}A_{M}^{S}G_{F}C_{M}A | | ACCAGG | | 001566 | A_{M}^{S}U_{F}^{S}G_{M}U_{F}U | | CCUGGU | |
| | | | | _{F}C_{M}C_{F}A_{M}C_{F}C_{M} | | GAACAUT | | | _{M}C_{F}C_{M}C_{F}U_{M}G_{F} | | GGUGCU | |
| | | | | A_{F}G_{M}G_{F}G_{M}A_{F} | | T | | | G_{M}U_{F}G_{M}G_{F}U_{M} | | CTT | |
| | | | | A_{M}C_{F}A_{M}U_{F}^{S}T_{D} | | | | | G_{F}C_{M}U_{F}C_{M}^{S}T_{D} | | | |
| | | | | ^{S}T_{D}-OH-3′ | | | | | ^{S}T_{D}-OH-3′ | | | |
| DT- | 36 | 54 | DTS- | 5′-OH- | 123 | AGCCUG | 462 | DTS- | 5′-PO4- | 275 | UGCAGC | 606 |
| 001012 | | | 001567 | A_{F}^{S}G_{M}C_{F}C_{M}U | | GUUGGA | | 001568 | U_{M}^{S}G_{F}^{S}C_{M}A_{F}G | | UUCCAAC | |
| | | | | _{F}G_{M}G_{F}U_{M}U_{F}G | | AGCUGC | | | _{M}C_{F}U_{M}U_{F}C_{M}C_{F} | | CAGGCUT | |
| | | | | _{M}G_{F}A_{M}A_{F}G_{M}C | | ATT | | | A_{M}A_{F}C_{M}C_{F}A_{M} | | T | |
| | | | | _{F}U_{M}G_{F}C_{M}A_{F}^{S}T | | | | | G_{F}G_{M}C_{F}U_{M}^{S}T_{D} | | | |
| | | | | _{D}^{S}T_{D}-OH-3′ | | | | | ^{S}T_{D}-OH-3′ | | | |
| DT- | 42 | 60 | DTS- | 5′-OH- | 124 | GUUGGA | 463 | DTS- | 5′-PO4- | 276 | UAAGCC | 588 |
| 001013 | | | 001569 | G_{F}^{S}U_{M}^{S}U_{F}G_{M}G | | AGCUGC | | 001570 | U_{M}^{S}A_{F}^{S}A_{M}G_{F}C | | UGCAGC | |
| | | | | _{F}A_{M}A_{F}G_{M}C_{F}U | | AGGCUU | | | _{M}C_{F}U_{M}G_{F}C_{M}A | | UUCCAAC | |
| | | | | _{M}G_{F}C_{M}A_{F}G_{M}G | | ATT | | | _{F}G_{M}C_{F}U_{M}U_{F}C | | TT | |
| | | | | _{F}C_{M}U_{F}U_{M}A_{F}^{S}T | | | | | _{M}C_{F}A_{M}A_{F}C_{M}^{S}T | | | |
| | | | | _{D}^{S}T_{D}-OH-3′ | | | | | _{D}^{S}T_{D}-OH-3′ | | | |
| DT- | 44 | 62 | DTS- | 5′-OH- | 125 | UGGAAG | 464 | DTS- | 5′-PO4- | 277 | ACUAAG | 525 |
| 001014 | | | 001571 | U_{F}^{S}G_{M}^{S}G_{F}A_{M}A | | CUGCAG | | 001572 | A_{M}^{S}C_{F}^{S}U_{M}A_{F}A | | CCUGCAG | |
| | | | | _{F}G_{M}C_{F}U_{M}G_{F}C | | GCUUAG | | | _{M}G_{F}C_{M}C_{F}U_{M}G | | CUUCCAT | |
| | | | | _{M}A_{F}G_{M}G_{F}C_{M}U | | UTT | | | _{F}C_{M}A_{F}G_{M}C_{F}U | | T | |
| | | | | _{F}U_{M}A_{F}G_{M}U_{F}^{S}T | | | | | _{M}U_{F}C_{M}C_{F}A_{M}^{S}T | | | |
| | | | | _{D}^{S}T_{D}-OH-3′ | | | | | _{D}^{S}T_{D}-OH-3′ | | | |
| DT- | 48 | 66 | DTS- | 5′-OH- | 126 | AGCUGC | 465 | DTS- | 5′-PO4- | 278 | ACAGAC | 517 |
| 001015 | | | 001573 | A_{F}^{S}G_{M}C_{F}U_{M}G | | AGGCUU | | 001574 | A_{M}^{S}C_{F}^{S}A_{M}G_{F}A | | UAAGCC | |
| | | | | _{F}C_{M}A_{F}G_{M}G_{F}C | | AGUCUG | | | _{M}C_{F}U_{M}A_{F}A_{M}G | | UGCAGC | |
| | | | | _{M}U_{F}U_{M}A_{F}G_{M}U | | UTT | | | _{F}C_{M}C_{F}U_{M}G_{F}C_{M} | | UTT | |
| | | | | _{F}C_{M}U_{F}G_{M}U_{F}^{S}T | | | | | A_{F}G_{M}C_{F}U_{M}^{S}T_{D} | | | |
| | | | | _{D}^{S}T_{D}-OH-3′ | | | | | ^{S}T_{D}-OH-3′ | | | |
| DT- | 74 | 92 | DTS- | 5′-OH- | 127 | GGGUCU | 466 | DTS- | 5′-PO4- | 279 | ACAGGG | 520 |
| 001016 | | | 001575 | G_{F}^{S}G_{M}^{S}G_{F}U_{M}C | | CUGACU | | 001576 | A_{M}^{S}C_{F}^{S}A_{M}G_{F}G | | CAGUCA | |
| | | | | _{F}U_{M}C_{F}U_{M}G_{F}A | | GCCCUGU | | | _{M}G_{F}C_{M}A_{F}G_{M}U | | GAGACCC | |
| | | | | _{M}C_{F}U_{M}G_{F}C_{M}C_{F} | | TT | | | _{F}C_{M}A_{F}G_{M}A_{F}G | | TT | |
| | | | | C_{M}U_{F}G_{M}U_{F}^{S}T_{D} | | | | | _{M}A_{F}C_{M}C_{F}C_{M}^{S}T | | | |
| | | | | T_{D}-OH-3′ | | | | | _{D}T_{D}-OH-3′ | | | |
| DT- | 96 | 114 | DTS- | 5′-OH- | 128 | GAGGGU | 467 | DTS- | 5′-PO4- | 280 | AUGUUA | 564 |
| 001017 | | | 001577 | G_{F}^{S}A_{M}^{S}G_{F}G_{M}G | | CUUGCCU | | 001578 | A_{M}^{S}U_{F}^{S}G_{M}U_{F}U | | AGGCAA | |
| | | | | _{F}U_{M}C_{F}U_{M}U_{F}G | | UAACAUT | | | _{M}A_{F}A_{M}G_{F}G_{M}C | | GACCCUC | |
| | | | | _{M}C_{F}C_{M}U_{F}U_{M}A | | T | | | _{F}A_{M}A_{F}G_{M}A_{F}C | | TT | |
| | | | | _{F}A_{M}C_{F}A_{M}U_{F}^{S}T | | | | | _{M}C_{F}C_{M}U_{F}C_{M}^{S}T | | | |
| | | | | _{D}^{S}T_{D}-OH-3′ | | | | | _{D}^{S}T_{D}-OH-3′ | | | |
| DT- | 101 | 119 | DTS- | 5′-OH- | 129 | UCUUGCC | 468 | DTS- | 5′-PO4- | 281 | AAGGGA | 509 |
| 001018 | | | 001579 | U_{F}^{S}C_{M}^{S}U_{F}U_{M}G | | UUAACA | | 001580 | A_{M}^{S}A_{F}^{S}G_{M}G_{F}G | | UGUUAA | |
| | | | | _{F}C_{M}C_{F}U_{M}U_{F}A | | UCCCUUT | | | _{M}A_{F}U_{M}G_{F}U_{M}U | | GGCAAG | |
| | | | | _{M}A_{F}C_{M}A_{F}U_{M}C | | T | | | _{F}A_{M}A_{F}G_{M}G_{F}C | | ATT | |
| | | | | _{F}C_{M}C_{F}U_{M}U_{F}^{S}T | | | | | _{M}A_{F}A_{M}G_{F}A_{M}^{S} | | | |
| | | | | _{D}^{S}T_{D}-OH-3′ | | | | | T_{D}^{S}T_{D}-OH-3′ | | | |
| DT- | 105 | 123 | DTS- | 5′-OH- | 130 | GCCUUA | 469 | DTS- | 5′-PO4- | 282 | AUGCAA | 557 |
| 001019 | | | 001581 | G_{F}^{S}C_{M}^{S}C_{F}U_{M}U | | ACAUCC | | 001582 | A_{M}^{S}U_{F}^{S}G_{M}C_{F} | | GGGAUG | |
| | | | | _{F}A_{M}A_{F}C_{M}A_{F}U_{M} | | CUUGCA | | | A_{M}A_{F}G_{M}G_{F}G_{M} | | UUAAGG | |
| | | | | C_{F}C_{M}C_{F}U_{M}U_{F} | | UTT | | | A_{F}U_{M}G_{F}U_{M}U_{F}A | | CTT | |
| | | | | G_{M}C_{F}A_{M}U_{F}^{S}T_{D} | | | | | _{M}A_{F}G_{M}G_{F}C_{M}^{S}T | | | |
| | | | | ^{S}T_{D}-OH-3′ | | | | | _{D}^{S}T_{D}-OH-3′ | | | |
| DT- | 107 | 125 | DTS- | 5′-OH- | 131 | CUUAAC | 470 | DTS- | 5′-PO4- | 283 | AAAUGC | 496 |
| 001020 | | | 001583 | C_{F}^{S}U_{M}^{S}U_{F}A_{M}A | | AUCCCUU | | 001584 | A_{M}^{S}A_{F}^{S}A_{M}U_{F}G | | AAGGGA | |
| | | | | _{F}C_{M}A_{F}U_{M}C_{F}C_{M} | | GCAUUUT | | | _{M}C_{F}A_{M}A_{F}G_{M}G | | UGUUAA | |
| | | | | C_{F}U_{M}U_{F}G_{M}C_{F} | | T | | | _{F}G_{M}A_{F}U_{M}G_{F}U | | GTT | |
| | | | | A_{M}U_{F}U_{M}U_{F}^{S}T_{D} | | | | | _{M}U_{F}A_{M}A_{F}G_{M}^{S} | | | |
| | | | | ^{S}T_{D}-OH-3′ | | | | | T_{D}^{S}T_{D}-OH-3′ | | | |
| DT- | 115 | 133 | DTS- | 5′-OH- | 132 | CCCUUGC | 471 | DTS- | 5′-PO4- | 284 | UUGCAG | 624 |
| 001021 | | | 001585 | C_{F}^{S}C_{M}^{S}C_{F}U_{M}U | | AUUUGG | | 001586 | U_{M}^{S}U_{F}^{S}G_{M}C_{F}A | | CCAAAU | |
| | | | | _{F}G_{M}C_{F}A_{M}U_{F}U | | CUGCAAT | | | _{M}G_{F}C_{M}C_{F}A_{M}A | | GCAAGG | |
| | | | | _{M}U_{F}G_{M}G_{F}C_{M}U | | T | | | _{F}A_{M}U_{F}G_{M}C_{F}A | | GTT | |
| | | | | _{F}G_{M}C_{F}A_{M}A_{F}^{S}T | | | | | _{M}A_{F}G_{M}G_{F}G_{M}^{S} | | | |
| | | | | _{D}^{S}T_{D}-OH-3′ | | | | | T_{D}^{S}T_{D}-OH-3′ | | | |
| DT- | 116 | 134 | DTS- | 5′-OH- | 133 | CCUUGCA | 472 | DTS- | 5′-PO4- | 285 | UUUGCA | 638 |
| 001022 | | | 001587 | C_{F}^{S}C_{M}^{S}U_{F}U_{M}G | | UUUGGC | | 001588 | U_{M}^{S}U_{F}^{S}U_{M}G_{F}C | | GCCAAA | |
| | | | | C_{M}A_{F}U_{M}U_{F}U | | UGCAAAT | | | _{M}A_{F}G_{M}C_{F}C_{M}A | | UGCAAG | |
| | | | | _{M}G_{F}G_{M}C_{F}U_{M}G | | T | | | _{F}A_{M}A_{F}U_{M}G_{F}C | | GTT | |
| | | | | _{F}C_{M}A_{F}A_{M}A_{F}^{S}T | | | | | _{M}A_{F}A_{M}G_{F}G_{M}^{S} | | | |
| | | | | _{D}^{S}T_{D}-OH-3′ | | | | | T_{D}^{S}T_{D}-OH-3′ | | | |
| DT- | 119 | 137 | DTS- | 5′-OH- | 134 | UGCAUU | 473 | DTS- | 5′-PO4- | 286 | UUCUUU | 621 |
| 001023 | | | 001589 | U_{F}^{S}G_{M}^{S}C_{F}A_{M}U | | UGGCUG | | 001590 | U_{M}^{S}U_{F}^{S}C_{M}U_{F}U | | GCAGCCA | |
| | | | | _{F}U_{M}U_{F}G_{M}G_{F}C | | CAAAGA | | | _{M}U_{F}G_{M}C_{F}A_{M}G | | AAUGCAT | |
| | | | | _{M}U_{F}G_{M}C_{F}A_{M}A | | ATT | | | _{F}C_{M}C_{F}A_{M}A_{F}A | | T | |
| | | | | _{F}A_{M}G_{F}A_{M}A_{F}^{S}T | | | | | _{M}U_{F}G_{M}C_{F}A_{M}^{S}T | | | |
| | | | | _{D}^{S}T_{D}-OH-3′ | | | | | _{D}^{S}T_{D}-OH-3′ | | | |
| DT- | 120 | 138 | DTS- | 5′-OH- | 135 | GCAUUU | 474 | DTS- | 5′-PO4- | 287 | UUUCUU | 636 |
| 001024 | | | 001591 | G_{F}^{S}C_{M}^{S}A_{F}U_{M}U | | GGCUGC | | 001592 | U_{M}^{S}U_{F}^{S}U_{M}C_{F}U | | UGCAGCC | |
| | | | | _{F}U_{M}G_{F}G_{M}C_{F}U | | AAAGAA | | | _{M}U_{F}U_{M}G_{F}C_{M}A | | AAAUGCT | |
| | | | | _{M}G_{F}C_{M}A_{F}A_{M}A | | ATT | | | G_{M}C_{F}C_{M}A_{F}A | | T | |
| | | | | _{F}G_{M}A_{F}A_{M}A_{F}^{S}T | | | | | _{M}A_{F}U_{M}G_{F}C_{M}^{S}T | | | |
| | | | | _{D}^{S}T_{D}-OH-3′ | | | | | _{D}^{S}T_{D}-OH-3′ | | | |
| DT- | 121 | 139 | DTS- | 5′-OH- | 136 | CAUUUG | 475 | DTS- | 5′-PO4- | 288 | AUUUCU | 574 |
| 001025 | | | 001593 | C_{F}^{S}A_{M}^{S}U_{F}U_{M}U | | GCUGCA | | 001594 | A_{M}^{S}U_{F}^{S}U_{M}U_{F}C | | UUGCAG | |
| | | | | _{F}G_{M}G_{F}C_{M}U_{F}G | | AAGAAA | | | _{M}U_{F}U_{M}U_{F}G_{M}C | | CCAAAU | |
| | | | | _{M}C_{F}A_{M}A_{F}A_{M}G | | UTT | | | _{F}A_{M}G_{F}C_{M}C_{F}A | | GTT | |
| | | | | _{F}A_{M}A_{F}A_{M}U_{F}^{S}T | | | | | _{M}A_{F}A_{M}U_{F}G_{M}^{S} | | | |
| | | | | _{D}^{S}T_{D}-OH-3′ | | | | | T_{D}^{S}T_{D}-OH-3′ | | | |
| DT- | 127 | 145 | DTS- | 5′-OH- | 137 | GCUGCA | 476 | DTS- | 5′-PO4- | 289 | AAGCAG | 507 |
| 001026 | | | 001595 | G_{F}^{S}C_{M}^{S}U_{F}G_{M}C | | AAGAAA | | 001596 | A_{M}^{S}A_{F}^{S}G_{M}C_{F}A | | AUUUCU | |
| | | | | _{F}A_{M}A_{F}A_{M}G_{F}A | | UCUGCU | | | _{M}G_{F}A_{M}U_{F}U_{M}U | | UUGCAG | |
| | | | | _{M}A_{F}A_{M}U_{F}C_{M}U | | UTT | | | C_{M}U_{F}U_{M}U_{F}G | | CTT | |
| | | | | G_{M}C_{F}U_{M}U_{F}^{S}T | | | | | _{M}C_{F}A_{M}G_{F}C_{M}^{S}T | | | |
| | | | | _{D}^{S}T_{D}-OH-3′ | | | | | _{D}^{S}T_{D}-OH-3′ | | | |
| DT- | 131 | 149 | DTS- | 5′-OH- | 138 | CAAAGA | 477 | DTS- | 5′-PO4- | 290 | UUCCAA | 615 |
| 001027 | | | 001597 | C_{F}^{S}A_{M}^{S}A_{F}A_{M}G | | AAUCUG | | 001598 | U_{M}^{S}U_{F}^{S}C_{M}C_{F}A | | GCAGAU | |
| | | | | _{F}A_{M}A_{F}A_{M}U_{F}C | | CUUGGA | | | _{M}A_{F}G_{M}C_{F}A_{M}G | | UUCUUU | |
| | | | | _{M}U_{F}G_{M}C_{F}U_{M}U | | ATT | | | _{F}A_{M}U_{F}U_{M}U_{F}C | | GTT | |
| | | | | _{F}G_{M}G_{F}A_{M}A_{F}^{S}T | | | | | _{M}U_{F}U_{M}U_{F}G^{S} | | | |
| | | | | _{D}^{S}T_{D}-OH-3′ | | | | | T_{D}^{S}T_{D}-OH-3′ | | | |
| DT- | 134 | 152 | DTS- | 5′-OH- | 139 | AGAAAU | 478 | DTS- | 5′-PO4- | 291 | UUCUUCC | 620 |
| 001028 | | | 001599 | A_{F}^{S}G_{M}^{S}A_{F}A_{M}A | | CUGCUU | | 001600 | U_{M}^{S}U_{F}^{S}C_{M}U_{F}U | | AAGCAG | |
| | | | | _{F}U_{M}C_{F}U_{M}G_{F}C | | GGAAGA | | | _{M}C_{F}C_{M}A_{F}A_{M}G | | AUUUCUT | |
| | | | | _{M}U_{F}U_{M}G_{F}G_{M}A | | ATT | | | _{F}C_{M}A_{F}G_{M}A_{F}U | | T | |
| | | | | _{F}A_{M}G_{F}A_{M}A_{F}^{S}T | | | | | _{M}U_{F}U_{M}C_{F}U_{M}^{S}T | | | |
| | | | | _{D}^{S}T_{D}-OH-3′ | | | | | _{D}^{S}T_{D}-OH-3′ | | | |
| DT- | 141 | 159 | DTS- | 5′-OH- | 140 | UGCUUG | 479 | DTS- | 5′-PO4- | 292 | UAACCCC | 587 |
| 001029 | | | 001601 | U_{F}^{S}G_{M}^{S}C_{F}U_{M}U | | GAAGAA | | 001602 | U_{M}^{S}A_{F}^{S}A_{M}C_{F}C | | UUCUUCC | |
| | | | | _{F}G_{M}G_{F}A_{M}A_{F}G | | GGGGUU | | | _{M}C_{F}C_{M}U_{F}U_{M}CE | | AAGCATT | |
| | | | | _{M}A_{F}A_{M}G_{F}G_{M}G | | ATT | | | U_{M}U_{F}C_{M}C_{F}A_{M} | | | |
| | | | | _{F}G_{M}U_{F}U_{M}A_{F}^{S}T | | | | | A_{F}G_{M}C_{F}A_{M}^{S}T_{D} | | | |
| | | | | _{D}^{S}T_{D}-OH-3′ | | | | | ^{S}T_{D}-OH-3′ | | | |
| DT- | 162 | 180 | DTS- | 5′-OH- | 141 | CUGUUU | 480 | DTS- | 5′-PO4- | 293 | UUUCUG | 633 |
| 001030 | | | 001603 | C_{F}^{S}U_{M}^{S}G_{F}U_{M}U | | GGCCGG | | 001604 | U_{M}^{S}U_{F}^{S}U_{M}C_{F}U | | CCCGGCC | |
| | | | | _{F}U_{M}G_{F}G_{M}C_{F}C | | GCAGAA | | | _{M}G_{F}C_{M}C_{F}C_{M}G_{F} | | AAACAGT | |
| | | | | _{M}G_{F}G_{M}G_{F}C_{M}A | | ATT | | | G_{M}C_{F}C_{M}A_{F}A_{M} | | T | |
| | | | | _{F}G_{M}A_{F}A_{M}A_{F}^{S}T | | | | | A_{F}C_{M}A_{F}G_{M}^{S}T_{D} | | | |
| | | | | _{D}^{S}T_{D}-OH-3′ | | | | | ^{S}T_{D}-OH-3′ | | | |
| DT- | 177 | 195 | DTS- | 5′-OH- | 142 | GAAACU | 481 | DTS- | 5′-PO4- | 294 | UUCUGC | 618 |
| 001031 | | | 001605 | G_{F}^{S}A_{M}^{S}A_{F}A_{M}C | | CCGCUGA | | 001606 | U_{M}^{S}U_{F}^{S}C_{M}U_{F}G | | UCAGCG | |
| | | | | _{F}U_{M}C_{F}C_{M}G_{F}C_{M} | | GCAGAAT | | | _{M}C_{F}U_{M}C_{F}A_{M}G | | GAGUUU | |
| | | | | U_{F}G_{M}A_{F}G_{M}C_{F} | | T | | | _{F}C_{M}G_{F}G_{M}A_{F}G | | CTT | |
| | | | | A_{M}G_{F}A_{M}A_{F}^{S}T_{D} | | | | | _{M}U_{F}U_{M}U_{F}C_{M}^{S}T | | | |
| | | | | ^{S}T_{D}-OH-3′ | | | | | _{D}^{S}T_{D}-OH-3′ | | | |
| DT- | 180 | 198 | DTS- | 5′-OH- | 143 | ACUCCGC | 482 | DTS- | 5′-PO4- | 295 | AAGUUC | 511 |
| 001032 | | | 001607 | A_{F}^{S}C_{M}^{S}U_{F}C_{M}C | | UGAGCA | | 001608 | A_{M}^{S}A_{F}^{S}G_{M}U_{F}U | | UGCUCA | |
| | | | | _{F}G_{M}C_{F}U_{M}G_{F}A | | GAACUUT | | | _{M}C_{F}U_{M}G_{F}C_{M}U | | GCGGAG | |
| | | | | _{M}G_{F}C_{M}A_{F}G_{M}A | | T | | | _{F}C_{M}A_{F}G_{M}C_{F}G | | UTT | |
| | | | | _{F}A_{M}C_{F}U_{M}U_{F}^{S}T | | | | | _{M}G_{F}A_{M}G_{F}U_{M} | | | |
| | | | | _{D}^{S}T_{D}-OH-3′ | | | | | T_{D}^{S}T_{D}-OH-3′ | | | |
| DT- | 190 | 208 | DTS- | 5′-OH- | 144 | GCAGAA | 483 | DTS- | 5′-PO4- | 296 | UUCUGG | 619 |
| 001033 | | | 001609 | G_{F}^{S}C_{M}^{S}A_{F}G_{M}A | | CUUGCCG | | 001610 | U_{M}^{S}U_{F}^{S}C_{M}U_{F}G | | CGGCAA | |
| | | | | _{F}A_{M}C_{F}U_{M}U_{F}G | | CCAGAAT | | | _{M}G_{F}C_{M}G_{F}G_{M}C | | GUUCUG | |
| | | | | _{M}C_{F}C_{M}G_{F}C_{M}CE | | T | | | _{F}A_{M}A_{F}G_{M}U_{F}U | | CTT | |
| | | | | A_{M}G_{F}A_{M}A_{F}^{S}T_{D} | | | | | _{M}C_{F}U_{M}G_{F}C_{M}^{S}T | | | |
| | | | | ^{S}T_{D}-OH-3′ | | | | | _{D}^{S}T_{D}-OH-3′ | | | |
| DT- | 191 | 209 | DTS- | 5′-OH- | 145 | CAGAAC | 484 | DTS- | 5′-PO4- | 297 | AUUCUG | 570 |
| 001034 | | | 001611 | C_{F}^{S}A_{M}^{S}G_{F}A_{M}A | | UUGCCGC | | 001612 | A_{M}^{S}U_{F}^{S}U_{M}C_{F}U | | GCGGCA | |
| | | | | _{F}C_{M}U_{F}U_{M}G_{F}C | | CAGAAUT | | | _{M}G_{F}G_{M}C_{F}G_{M}G | | AGUUCU | |
| | | | | _{M}C_{F}G_{M}C_{F}C_{M}A_{F} | | T | | | _{F}C_{M}A_{F}A_{M}G_{F}U | | GTT | |
| | | | | G_{M}A_{F}A_{M}U_{F}^{S}T_{D} | | | | | _{M}U_{F}C_{M}U_{F}G_{M}^{S}T | | | |
| | | | | ^{S}T_{D}-OH-3′ | | | | | _{D}^{S}T_{D}-OH-3′ | | | |
| DT- | 474 | 492 | DTS- | 5′-OH- | 146 | GGCUCU | 485 | DTS- | 5′-PO4- | 317 | GAAGAA | 580 |
| 001103 | | | 001731 | G_{F}^{S}G_{M}^{S}C_{F}U_{M}C | | GUUCCU | | 001732 | G_{M}^{S}A_{F}^{S}A_{M}G_{F}A | | CAGGAA | |
| | | | | _{F}U_{M}G_{F}U_{M}U_{F}C | | GUUCUU | | | _{M}A_{F}C_{M}A_{F}G_{M}G | | CAGAGCC | |
| | | | | _{M}C_{F}U_{M}G_{F}U_{M}U | | CTT | | | _{F}A_{M}A_{F}C_{M}A_{F}G | | TT | |
| | | | | _{F}C_{M}U_{F}U_{M}CE^{S}T | | | | | _{M}A_{F}G_{M}C_{F}C_{M}^{S}T | | | |
| | | | | _{D}^{S}T_{D}-OH-3′ | | | | | _{D}^{S}T_{D}-OH-3′ | | | |
| DT- | 874 | 892 | DTS- | 5′-OH- | 147 | ACCUAU | 486 | DTS- | 5′-PO4- | 318 | AAAAGU | 491 |
| 001104 | | | 001733 | A_{F}^{S}C_{M}^{S}C_{F}U_{M}A | | UUAUAA | | 001734 | A_{M}^{S}A_{F}^{S}A_{M}A_{F}G | | GUUAUA | |
| | | | | _{F}U_{M}U_{F}U_{M}A_{F}U | | CACUUU | | | _{M}U_{F}G_{M}U_{F}U_{M}A | | AAUAGG | |
| | | | | _{M}A_{F}A_{M}C_{F}A_{M}C | | UTT | | | _{F}U_{M}A_{F}A_{M}A_{F}U | | UTT | |
| | 1562 | 1580 | DTS- | _{F}U_{M}U_{F}U_{M}U_{F}^{S}T | | | | | _{M}A_{F}G_{M}G_{F}U_{M}^{S} | | | |
| | | | 001735 | _{D}^{S}T_{D}-OH-3′ | | | | | T_{D}^{S}T_{D}-OH-3′ | | | |
| DT- | | | | 5′-OH- | 148 | ACAAUA | 487 | DTS- | 5′-PO4- | 319 | UUUGAG | 637 |
| 001105 | | | | A_{F}^{S}C_{M}^{S}A_{F}A_{M}U | | AAUAAA | | 001736 | U_{M}^{S}U_{F}^{S}U_{M}G_{F}A | | AUUUAU | |
| | | | | _{F}A_{M}A_{F}A_{M}U_{F}A | | UCUCAA | | | _{M}G_{F}A_{M}U_{F}U_{M}U | | UUAUUG | |
| | | | | _{M}A_{F}A_{M}U_{F}C_{M}U | | ATT | | | _{F}A_{M}U_{F}U_{M}U_{F}A | | UTT | |
| | | | | _{F}C_{M}A_{F}A_{M}A_{F}^{S}T | | | | | _{M}U_{F}U_{M}G_{F}U_{M}^{S} | | | |
| | | | | _{D}^{S}T_{D}-OH-3′ | | | | | T_{D}^{S}T_{D}-OH-3′ | | | |
| DT- | 989 | 1007 | DTS- | 5′-OH- | 149 | CCUCGUG | 488 | DTS- | 5′-PO4- | 320 | UUUAAG | 630 |
| 001106 | | | 001737 | C_{F}^{S}C_{M}^{S}U_{F}C_{M}G | | UUGAAU | | 001738 | U_{M}^{S}U_{F}^{S}U_{M}A_{F}A | | AUUCAA | |
| | | | | _{F}U_{M}G_{F}U_{M}U_{F}G | | CUUAAAT | | | _{M}G_{F}A_{M}U_{F}U_{M}C | | CACGAG | |
| | | | | _{M}A_{F}A_{M}U_{F}C_{M}U | | T | | | _{F}A_{M}A_{F}C_{M}A_{F}C | | GTT | |
| | | | | _{F}U_{M}A_{F}A_{M}A_{F}^{S}T | | | | | _{M}G_{F}A_{M}G_{F}G_{M}^{S} | | | |
| | | | | _{D}^{S}T_{D}-OH-3′ | | | | | T_{D}^{S}T_{D}-OH-3′ | | | |
| DT- | 1785 | 1803 | DTS- | 5′-OH- | 150 | AUACCA | 489 | DTS- | 5′-PO4- | 321 | UAGUCC | 597 |
| 001107 | | | 001739 | A_{F}^{S}U_{M}^{S}A_{F}C_{M}C | | ACUGUG | | 001740 | U_{M}^{S}A_{F}^{S}G_{M}U_{F}C | | ACACAG | |
| | | | | _{F}A_{M}A_{F}C_{M}U_{F}G | | UGGACU | | | _{M}C_{F}A_{M}C_{F}A_{M}C_{F} | | UUGGUA | |
| | | | | _{M}U_{F}G_{M}U_{F}G_{M}G | | ATT | | | A_{M}G_{F}U_{M}U_{F}G_{M} | | UTT | |
| | | | | _{F}A_{M}C_{F}U_{M}A_{F}^{S}T | | | | | G_{F}U_{M}A_{F}U_{M}T_{D} | | | |
| | | | | _{D}^{S}T_{D}-OH-3′ | | | | | ^{S}T_{D}-OH-3′ | | | |
| DT- | 872 | 890 | DTS- | 5′-OH- | 151 | AAACCU | 490 | DTS- | 5′-PO4- | 322 | AAGUGU | 510 |
| 001108 | | | 001741 | A_{F}^{S}A_{M}^{S}A_{F}C_{M}C | | AUUUAU | | 001742 | A_{M}^{S}A_{F}^{S}G_{M}U_{F}G | | UAUAAA | |
| | | | | _{F}U_{M}A_{F}U_{M}U_{F}U | | AACACU | | | _{M}U_{F}U_{M}A_{F}U_{M}A | | UAGGUU | |
| | | | | _{M}A_{F}U_{M}A_{F}A_{M}C | | UTT | | | _{F}A_{M}A_{F}U_{M}A_{F}G | | UTT | |
| | | | | _{F}A_{M}C_{F}U_{M}U_{F}^{S}T | | | | | _{M}G_{F}U_{M}U_{F}U_{M}^{S} | | | |
| | | | | _{D}^{S}T_{D}-OH-3′ | | | | | T_{D}^{S}T_{D}-OH-3′ | | | |
| DT- | 735 | 755 | DTS- | 5′-OH- | 748 | GGGAGG | 1069 | DTS- | 5′-PO4- | 881 | UUUCUG | 1168 |
| 001209 | | | 001845 | G_{F}^{S}G_{M}^{S}G_{F}A_{M}G | | AAGGGA | | 001846 | U_{M}^{S}U_{F}^{S}U_{M}C_{F}U | | UUUUCCC | |
| | | | | _{F}G_{M}A_{F}A_{M}G_{F}G | | AAACAG | | | _{M}G_{F}U_{M}U_{F}U_{M}U | | UUCCUCC | |
| | | | | _{M}G_{F}A_{M}A_{F}A_{M}A | | AAA | | | _{F}C_{M}C_{F}C_{M}U_{F}U_{M} | | CUU | |
| | | | | _{F}C_{M}A_{F}G_{M}A_{F}^{S}A | | | | | C_{F}C_{M}U_{F}C_{M}C_{F} | | | |
| | | | | _{M}A_{F}-OH-3′ | | | | | C_{M}^{S}U_{M}U_{M}- | | | |
| | | | | | | | | | OH-3′ | | | |
| DT- | 777 | 797 | DTS- | 5′-OH- | 749 | AGCCCAA | 993 | DTS- | 5′-PO4- | 882 | UUGAGU | 1164 |
| 001210 | | | 001847 | A_{F}^{S}G_{M}^{S}C_{F}C_{M}C | | AAUCCCA | | 001848 | U_{M}^{S}U_{F}^{S}G_{M}A_{F}G | | UUGGGA | |
| | | | | _{F}A_{M}A_{F}A_{M}A_{F}U | | AACUCA | | | _{M}U_{F}U_{M}U_{F}G_{M}G | | UUUUGG | |
| | | | | _{M}C_{F}C_{M}C_{F}A_{M}A_{F} | | A | | | G_{M}A_{F}U_{M}U_{F}U | | GCUCG | |
| | | | | A_{M}C_{F}U_{M}C_{F}A_{M} | | | | | _{M}U_{F}G_{M}G_{F}G_{M}C | | | |
| | | | | A_{F}-OH-3′ | | | | | _{F}U_{M}^{S}C_{M}^{S}G_{M}- | | | |
| | | | | | | | | | OH-3′ | | | |
| DT- | 831 | 851 | DTS- | 5′-OH- | 750 | UUGCUG | 1108 | DTS- | 5′-PO4- | 883 | UACAUC | 1156 |
| 001211 | | | 001849 | U_{F}^{S}U_{M}^{S}G_{F}C_{M}U | | UUGAUU | | 001850 | U_{M}^{S}A_{F}^{S}C_{M}A_{F}U | | UUCAAU | |
| | | | | _{F}G_{M}U_{F}U_{M}G_{F}A | | GAAGAU | | | _{M}C_{F}U_{M}U_{F}C_{M}A | | CAACAGC | |
| | | | | _{M}U_{F}U_{M}G_{F}A_{M}A | | GUA | | | _{F}A_{M}U_{F}C_{M}A_{F}A | | AACC | |
| | | | | _{F}G_{M}A_{F}U_{M}G_{F}^{S}U | | | | | _{M}C_{F}A_{M}G_{F}C_{M}A | | | |
| | | | | _{M}A_{F}-OH-3′ | | | | | _{F}A_{M}^{S}C_{M}^{S}C_{M}- | | | |
| | | | | | | | | | OH-3′ | | | |
| DT- | 948 | 968 | DTS- | 5′-OH- | 751 | GAGCCU | 1051 | DTS- | 5′-PO4- | 884 | UUAGCU | 1158 |
| 001212 | | | 001851 | G_{F}^{S}A_{M}^{S}G_{F}C_{M}C | | UAAAGA | | 001852 | U_{M}^{S}U_{F}^{S}A_{M}G_{F}C | | ACUUCU | |
| | | | | _{F}U_{M}U_{F}A_{M}A_{F}A | | AGUAGC | | | _{M}U_{F}A_{M}C_{F}U_{M}U | | UUAAGG | |
| | | | | _{M}G_{F}A_{M}A_{F}G_{M}U | | UAA | | | C_{M}U_{F}U_{M}U_{F}A | | CUCAA | |
| | | | | _{F}A_{M}G_{F}C_{M}U_{F}^{S}A | | | | | _{M}A_{F}G_{M}G_{F}C_{M}U | | | |
| | | | | _{M}^{S}A_{F}-OH-3′ | | | | | _{F}C_{M}^{S}A_{M}^{S}A_{M}- | | | |
| | | | | | | | | | OH-3′ | | | |
| DT- | 735 | 755 | DTS- | 5′-OH- | 748 | GGGAGG | 1069 | DTS- | 5′-VP- | 885 | UUUCUG | 1168 |
| 001213 | | | 001845 | G_{F}^{S}G_{M}^{S}G_{F}A_{M}G | | AAGGGA | | 001853 | U_{M}^{S}U_{F}^{S}U_{M}C_{F}U | | UUUUCCC | |
| | | | | G_{M}A_{F}A_{M}G_{F}G | | AAACAG | | | _{M}G_{F}U_{M}U_{F}U_{M}U | | UUCCUCC | |
| | | | | _{M}G_{F}A_{M}A_{F}A_{M}A | | AAA | | | C_{M}C_{F}C_{M}U_{F}U_{M} | | CUU | |
| | | | | _{F}C_{M}A_{F}G_{M}A_{F}^{S}A | | | | | C_{F}C_{M}U_{F}C_{M}C_{F} | | | |
| | | | | _{M}A_{F}-OH-3′ | | | | | C_{M}^{S}U_{M}^{S}U_{M}- | | | |
| | | | | | | | | | OH-3′ | | | |
| DT- | 777 | 797 | DTS- | 5′-OH- | 749 | AGCCCAA | 993 | DTS- | 5′-VP- | 886 | UUGAGU | 1164 |
| 001214 | | | 001847 | A_{F}^{S}G_{M}^{S}C_{F}C_{M}C | | AAUCCCA | | 001854 | U_{M}^{S}U_{F}^{S}G_{M}A_{F}G | | UUGGGA | |
| | | | | _{F}A_{M}A_{F}A_{M}A_{F}U | | AACUCAA | | | _{M}U_{F}U_{M}U_{F}G_{M}G | | UUUUGG | |
| | | | | _{M}C_{F}C_{M}C_{F}A_{M}A_{F} | | | | | G_{M}A_{F}U_{M}U_{F}U | | GCUCG | |
| | | | | A_{M}C_{F}U_{M}C_{F}A_{M} | | | | | _{M}U_{F}G_{M}G_{F}G_{M}C | | | |
| | | | | A_{F}-OH-3′ | | | | | _{F}U_{M}^{S}C_{M}^{S}G_{M}- | | | |
| | | | | | | | | | OH-3′ | | | |
| DT- | 831 | 851 | DTS- | 5′-OH- | 750 | UUGCUG | 1108 | DTS- | 5′-VP- | 887 | UACAUC | 1156 |
| 001215 | | | 001849 | U_{F}^{S}U_{M}^{S}G_{F}C_{M}U | | UUGAUU | | 001855 | U_{M}^{S}A_{F}^{S}C_{M}A_{F}U | | UUCAAU | |
| | | | | G_{M}U_{F}U_{M}G_{F}A | | GAAGAU | | | _{M}C_{F}U_{M}U_{F}C_{M}A | | CAACAGC | |
| | | | | _{M}U_{F}U_{M}G_{F}A_{M}A | | GUA | | | _{F}A_{M}U_{F}C_{M}A_{F}A | | AACC | |
| | | | | _{F}G_{M}A_{F}U_{M}G_{F}^{S}U | | | | | _{M}C_{F}A_{M}G_{F}C_{M}A | | | |
| | | | | ^{S}A_{F}-OH-3′ | | | | | _{F}A_{M}^{S}C_{M}C_{M}- | | | |
| | | | | | | | | | OH-3′ | | | |
| DT- | 948 | 968 | DTS- | 5′-OH- | 751 | GAGCCU | 1051 | DTS- | 5′-VP- | 888 | UUAGCU | 1158 |
| 001216 | | | 001851 | G_{F}^{S}A_{M}^{S}G_{F}C_{M}C | | UAAAGA | | 001856 | U_{M}^{S}U_{F}^{S}A_{M}G_{F}C | | ACUUCU | |
| | | | | _{F}U_{M}U_{F}A_{M}A_{F}A | | AGUAGC | | | _{M}U_{F}A_{M}C_{F}U_{M}U | | UUAAGG | |
| | | | | _{M}G_{F}A_{M}A_{F}G_{M}U | | UAA | | | C_{M}U_{F}U_{M}U_{F}A | | CUCAA | |
| | | | | _{F}A_{M}G_{F}C_{M}U_{F}^{S}A | | | | | _{M}A_{F}G_{M}G_{F}C_{M}U | | | |
| | | | | _{M}A_{F}-OH-3′ | | | | | _{F}C_{M}^{S}A_{M}^{S}A_{M}- | | | |
| | | | | | | | | | OH-3′ | | | |
| DT- | 973 | 993 | DTS- | 5′-OH- | 756 | CUUUAC | 1047 | DTS- | 5′-PO4- | 889 | UUAUAC | 1160 |
| 001225 | | | 001861 | C_{F}^{S}U_{M}^{S}U_{F}U_{M}A | | AUCCUA | | 001862 | U_{M}^{S}U_{F}^{S}A_{M}U_{F}A | | UGUUAG | |
| | | | | C_{M}A_{F}U_{M}C_{F}C_{M} | | ACAGUA | | | _{M}C_{F}U_{M}G_{F}U_{M}U | | GAUGUA | |
| | | | | U_{F}A_{M}A_{F}C_{M}A_{F} | | UAA | | | _{F}A_{M}G_{F}G_{M}A_{F}U | | AAGUU | |
| | | | | G_{M}U_{F}A_{M}U_{F}^{S}A | | | | | _{M}G_{F}U_{M}A_{F}A_{M}A | | | |
| | | | | _{M}^{S}A_{F}-OH-3′ | | | | | G_{M}^{S}U_{M}^{S}U_{M}- | | | |
| | | | | | | | | | OH-3′ | | | |
| DT- | 1037 | 1057 | DTS- | 5′-OH- | 757 | UUUUAC | 1111 | DTS- | 5′-PO4- | 890 | UUAUCU | 1161 |
| 001226 | | | 001863 | U_{F}^{S}U_{M}^{S}U_{F}U_{M}A | | CCAGAA | | 001864 | U_{M}^{S}U_{F}^{S}A_{M}U_{F}C | | UAUUUC | |
| | | | | C_{M}C_{F}C_{M}A_{F}G_{M} | | AUAAGA | | | _{M}U_{F}U_{M}A_{F}U_{M}U | | UGGGUA | |
| | | | | A_{F}A_{M}A_{F}U_{M}A_{F} | | UAA | | | _{F}U_{M}C_{F}U_{M}G_{F}G | | AAACA | |
| | | | | A_{M}G_{F}A_{M}U_{F}^{S}A | | | | | _{M}G_{F}U_{M}A_{F}A_{M}A | | | |
| | | | | _{M}^{S}A_{F}-OH-3′ | | | | | _{F}A_{M}^{S}C_{M}^{S}A_{M}- | | | |
| | | | | | | | | | OH-3′ | | | |
| DT- | 1693 | 1713 | DTS- | 5′-OH- | 758 | GGCCACC | 1066 | DTS- | 5′-PO4- | 891 | AUACAU | 1136 |
| 001227 | | | 001865 | G_{F}^{S}G_{M}^{S}C_{F}C_{M}A | | AACUGU | | 001866 | A_{M}^{S}U_{F}^{S}A_{M}C_{F}A | | CUACAG | |
| | | | | _{F}C_{M}C_{F}A_{M}A_{F}C_{M} | | AGAUGU | | | _{M}U_{F}C_{M}U_{F}A_{M}C | | UUGGUG | |
| | | | | U_{F}G_{M}U_{F}A_{M}G_{F} | | AU | | | _{F}A_{M}G_{F}U_{M}U_{F}G | | GCCAA | |
| | | | | A_{M}U_{F}G_{M}U_{F}^{S}A | | | | | _{M}G_{F}U_{M}G_{F}G_{M}C | | | |
| | | | | _{M}U_{F}-OH-3′ | | | | | _{F}C_{M}^{S}A_{M}^{S}A_{M}- | | | |
| | | | | | | | | | OH-3′ | | | |
| DT- | 1755 | 1775 | DTS- | 5′-OH- | 759 | UUUGCU | 1110 | DTS- | 5′-PO4- | 892 | AAUCAG | 1122 |
| 001228 | | | 001867 | U_{F}^{S}U_{M}^{S}U_{F}G_{M}C | | UUGCAU | | 001868 | A_{M}^{S}A_{F}^{S}U_{M}C_{F}A | | AAAAUG | |
| | | | | _{F}U_{M}U_{F}U_{M}G_{F}C | | UUUCUG | | | _{M}G_{F}A_{M}A_{F}A_{M}A | | CAAAGC | |
| | | | | _{M}A_{F}U_{M}U_{F}U_{M}U | | AUU | | | _{F}U_{M}G_{F}C_{M}A_{F}A | | AAAAA | |
| | | | | _{F}C_{M}U_{F}G_{M}A_{F}^{S}U | | | | | _{M}A_{F}G_{M}C_{F}A_{M}A | | | |
| | | | | _{M}U_{F}-OH-3′ | | | | | _{F}A_{M}^{S}A_{M}^{S}A_{M}- | | | |
| | | | | | | | | | OH-3′ | | | |
| DT- | 1780 | 1800 | DTS- | 5′-OH- | 760 | ACCAACU | 986 | DTS- | 5′-PO4- | 893 | AUCUUA | 1142 |
| 001229 | | | 001869 | A_{F}^{S}C_{M}^{S}C_{F}A_{M}A | | GUGUGG | | 001870 | A_{M}^{S}U_{F}^{S}C_{M}U_{F}U | | GUCCACA | |
| | | | | _{F}C_{M}U_{F}G_{M}U_{F}G | | ACUAAG | | | _{M}A_{F}G_{M}U_{F}C_{M}C | | CAGUUG | |
| | | | | _{M}U_{F}G_{M}G_{F}A_{M}C | | AU | | | _{F}A_{M}C_{F}A_{M}C_{F}A | | GUAU | |
| | | | | _{F}U_{M}A_{F}A_{M}GE^{S}A | | | | | _{M}G_{F}U_{M}U_{F}G_{M}G | | | |
| | | | | ^{S}U_{F}-OH-3′ | | | | | _{F}U_{M}^{S}A_{M}^{S}U_{M}- | | | |
| | | | | | | | | | OH-3′ | | | |
| DT- | 208 | 218 | DTS- | 5′-OH- | 787 | AUGCUCC | 1000 | DTS- | 5′-PO4- | 910 | ACUCAGC | |
| 001268 | | | 001920 | A_{F}^{S}U_{M}^{S}G_{F}C_{M}U | | UCCUGU | | 001921 | A_{M}^{S}C_{F}^{S}U_{M}C_{F}A | | AACAGG | |
| | | | | _{F}C_{M}C_{F}U_{M}C_{F}C_{M} | | UGCUGA | | | _{M}G_{F}C_{M}A_{F}A_{M}C | | AGGAGC | |
| | | | | U_{F}G_{M}U_{F}U_{M}G_{F} | | GU | | | _{F}A_{M}G_{F}G_{M}A_{F}G | | AUUC | |
| | | | | C_{M}U_{F}G_{M}A_{F}^{S}G | | | | | _{M}G_{F}A_{M}G_{F}C_{M}A | | | |
| | | | | _{M}^{S}U_{F}-OH-3′ | | | | | U_{M}^{S}U_{M}^{S}C_{M}- | | | |
| | | | | | | | | | OH-3′ | | | 1127 |
| DT- | 210 | 230 | DTS- | 5′-OH- | 788 | GCUCCUC | | DTS- | 5′-PO4- | | AUACUC | |
| 001269 | | | 001922 | G_{F}^{S}C_{M}^{S}U_{F}C_{M}C | | CUGUUG | | | A_{M}^{S}U_{F}^{S}A_{M}C_{F}U | | AGCAAC | |
| | | | | _{F}U_{M}C_{F}C_{M}U_{F}G | | CUGAGU | | | _{M}C_{F}A_{M}G_{F}C_{M}A | | AGGAGG | |
| | | | | _{M}U_{F}U_{M}G_{F}C_{M}U | | AU | | | _{F}A_{M}C_{F}A_{M}G_{F}G | | | |
| | | | | G_{M}A_{F}G_{M}U_{F}^{S}A | | | | | _{M}A_{F}G_{M}G_{F}A_{M}G | | | |
| | | | | _{M}^{S}U_{F}-OH-3′ | | | | | C_{M}^{S}A_{M}^{S}U_{M}- | | | |
| | | | | | | | 1060 | 001923 | OH-3′ | 911 | AGCAU | 1138 |
| DT- | 211 | 231 | DTS- | 5′-OH- | 789 | CUCCUCC | 1034 | DTS- | 5′-PO4- | 912 | GAUACU | 1153 |
| 001270 | | | 001924 | C_{F}^{S}U_{M}^{S}C_{F}C_{M}U | | UGUUGC | | 001925 | G_{M}^{S}A_{F}^{S}U_{M}A_{F}C | | CAGCAAC | |
| | | | | _{F}C_{M}C_{F}U_{M}G_{F}U | | UGAGUA | | | _{M}U_{F}C_{M}A_{F}G_{M}C | | AGGAGG | |
| | | | | _{M}U_{F}G_{M}C_{F}U_{M}G | | UC | | | _{F}A_{M}A_{F}C_{M}A_{F}G | | AGCA | |
| | | | | _{F}A_{M}G_{F}U_{M}A_{F}^{S}U | | | | | _{M}G_{F}A_{M}G_{F}G_{M}A | | | |
| | | | | _{M}C_{F}-OH-3′ | | | | | G_{M}^{S}C_{M}^{S}A_{M}- | | | |
| | | | | | | | | | OH-3′ | | | |
| DT- | 212 | 232 | DTS- | 5′-OH- | 790 | UCCUCCU | 1088 | DTS- | 5′-PO4- | 913 | UGAUAC | 1157 |
| 001271 | | | 001926 | U_{F}^{S}C_{M}^{S}C_{F}U_{M}C | | GUUGCU | | 001927 | U_{M}^{S}G_{F}^{S}A_{M}U_{F}A | | UCAGCA | |
| | | | | _{F}C_{M}U_{F}G_{M}U_{F}U | | GAGUAU | | | _{M}C_{F}U_{M}C_{F}A_{M}G | | ACAGGA | |
| | | | | _{M}G_{F}C_{M}U_{F}G_{M}A | | CA | | | _{F}C_{M}A_{F}A_{M}C_{F}A | | GGAGC | |
| | | | | _{F}G_{M}U_{F}A_{M}U_{F}^{S}C | | | | | _{M}G_{F}G_{M}A_{F}G_{M}G | | | |
| | | | | _{M}A_{F}-OH-3′ | | | | | _{F}A_{M}^{S}G_{M}^{S}C_{M}- | | | |
| | | | | | | | | | OH-3′ | | | |
| DT- | 214 | 234 | DTS- | 5′-OH- | 791 | CUCCUGU | 1037 | DTS- | 5′-PO4- | 914 | GAUGAU | 1154 |
| 001272 | | | 001928 | C_{F}^{S}U_{M}^{S}C_{F}C_{M}U | | UGCUGA | | 001929 | G_{M}^{S}A_{F}^{S}U_{M}G_{F}A | | ACUCAGC | |
| | | | | _{F}G_{M}U_{F}U_{M}G_{F}C | | GUAUCA | | | _{M}U_{F}A_{M}C_{F}U_{M}C | | AACAGG | |
| | | | | _{M}U_{F}G_{M}A_{F}G_{M}U | | UC | | | _{F}A_{M}G_{F}C_{M}A_{F}A | | AGGA | |
| | | | | _{F}A_{M}U_{F}C_{M}A_{F}^{S}U | | | | | _{M}C_{F}A_{M}G_{F}G_{M}A | | | |
| | | | | _{M}^{S}C_{F}-OH-3′ | | | | | G_{M}^{S}G_{M}^{S}A_{M}- | | | |
| | | | | | | | | | OH-3′ | | | |
| DT- | 215 | 235 | DTS- | 5′-OH- | 792 | UCCUGU | 1091 | DTS- | 5′-PO4- | 915 | CGAUGA | 1151 |
| 001273 | | | 001930 | U_{F}^{S}C_{M}^{S}C_{F}U_{M}G | | UGCUGA | | 001931 | C_{M}^{S}GE^{S}A_{M}U_{F}G | | UACUCA | |
| | | | | _{F}U_{M}U_{F}G_{M}C_{F}U | | GUAUCA | | | _{M}A_{F}U_{M}A_{F}C_{M}U | | GCAACA | |
| | | | | _{M}G_{F}A_{M}G_{F}U_{M}A | | UCG | | | C_{M}A_{F}G_{M}C_{F}A | | GGAGG | |
| | | | | _{F}U_{M}C_{F}A_{M}U_{F}^{S}C | | | | | _{M}A_{F}C_{M}A_{F}G_{M}G | | | |
| | | | | _{M}G_{F}-OH-3′ | | | | | _{F}A_{M}^{S}G_{M}^{S}G_{M}- | | | |
| | | | | | | | | | OH-3′ | | | |
| DT- | 217 | 237 | DTS- | 5′-OH- | 793 | CUGUUG | 1045 | DTS- | 5′-PO4- | 916 | GACGAU | 1152 |
| 001274 | | | 001932 | C_{F}^{S}U_{M}^{S}G_{F}U_{M}U | | CUGAGU | | 001933 | G_{M}^{S}A_{F}^{S}C_{M}G_{F}A | | GAUACU | |
| | | | | _{F}G_{M}C_{F}U_{M}G_{F}A | | AUCAUC | | | _{M}U_{F}G_{M}A_{F}U_{M}A | | CAGCAAC | |
| | | | | _{M}G_{F}U_{M}A_{F}U_{M}C | | GUC | | | _{F}C_{M}U_{F}C_{M}A_{F}G | | AGGA | |
| | | | | _{F}A_{M}U_{F}C_{M}GE^{S}U | | | | | _{M}C_{F}A_{M}A_{F}C_{M}A | | | |
| | | | | _{M}C_{F}-OH-3′ | | | | | _{F}G_{M}G_{M}^{S}A_{M}- | | | |
| | | | | | | | | | OH-3′ | | | |
| DT- | 218 | 238 | DTS- | 5′-OH- | 794 | UGUUGC | 1103 | DTS- | 5′-PO4- | 917 | GGACGA | 1155 |
| 001275 | | | 001934 | U_{F}^{S}G_{M}^{S}U_{F}U_{M}G | | UGAGUA | | 001935 | G_{M}^{S}G_{F}^{S}A_{M}C_{F}G | | UGAUAC | |
| | | | | _{F}C_{M}U_{F}G_{M}A_{F}G | | UCAUCG | | | _{M}A_{F}U_{M}G_{F}A_{M}U | | UCAGCA | |
| | | | | _{M}U_{F}A_{M}U_{F}C_{M}A | | UCC | | | _{F}A_{M}C_{F}U_{M}C_{F}A | | ACAGG | |
| | | | | _{F}U_{M}C_{F}G_{M}U_{F}^{S}C | | | | | _{M}G_{F}C_{M}A_{F}A_{M}C | | | |
| | | | | _{M}^{S}C_{F}-OH-3′ | | | | | _{F}A_{M}^{S}G_{M}^{S}G_{M}- | | | |
| | | | | | | | | | OH-3′ | | | |
| DT- | 225 | 245 | DTS- | 5′-OH- | 800 | GAGUAU | 1054 | DTS- | 5′-PO4- | 918 | ACGUGG | 1126 |
| 001284 | | | 001941 | G_{F}^{S}A_{M}^{S}G_{F}U_{M}A | | CAUCGUC | | 001942 | A_{M}^{S}C_{F}^{S}G_{M}U_{F}G | | AGGACG | |
| | | | | _{F}U_{M}C_{F}A_{M}U_{F}C | | CUCCACG | | | _{M}G_{F}A_{M}G_{F}G_{M}A | | AUGAUA | |
| | | | | _{M}G_{F}U_{M}C_{F}C_{M}U | | U | | | _{F}C_{M}G_{F}A_{M}U_{F}G | | CUCAG | |
| | | | | _{F}C_{M}C_{F}A_{M}C_{F}^{S}G | | | | | _{M}A_{F}U_{M}A_{F}C_{M}U | | | |
| | | | | _{M}^{S}U_{F}-OH-3′ | | | | | _{F}C_{M}^{S}A_{M}^{S}G_{M}- | | | |
| | | | | | | | | | OH-3′ | | | |
| DT- | 243 | 263 | DTS- | 5′-OH- | 801 | CGUCGCG | 1028 | DTS- | 5′-PO4- | 919 | AGCAGC | 1131 |
| 001285 | | | 001943 | C_{F}^{S}G_{M}^{S}U_{F}C_{M}G | | GUGCUG | | 001944 | A_{M}^{S}G_{F}^{S}C_{M}A_{F}G | | ACCAGCA | |
| | | | | _{F}C_{M}G_{F}G_{M}U_{F}G | | GUGCUG | | | _{M}C_{F}A_{M}C_{F}C_{M}A_{F} | | CCGCGAC | |
| | | | | _{M}C_{F}U_{M}G_{F}G_{M}U | | CU | | | G_{M}C_{F}A_{M}C_{F}C_{M} | | GUG | |
| | | | | G_{M}C_{F}U_{M}GE^{S}C | | | | | G_{F}C_{M}G_{F}A_{M}C_{F} | | | |
| | | | | _{M}U_{F}-OH-3′ | | | | | G_{M}^{S}U_{M}^{S}G_{M}- | | | |
| | | | | | | | | | OH-3′ | | | |
| DT- | 251 | 271 | DTS- | 5′-OH- | 802 | UGCUGG | 1097 | DTS- | 5′-PO4- | 920 | AGACGA | 1128 |
| 001286 | | | 001945 | U_{F}^{S}G_{M}^{S}C_{F}U_{M}G | | UGCUGC | | 001946 | A_{M}^{S}G_{F}^{S}A_{M}C_{F}G | | ACAGCA | |
| | | | | _{F}G_{M}U_{F}G_{M}C_{F}U | | UGUUCG | | | _{M}A_{F}A_{M}C_{F}A_{M}G | | GCACCAG | |
| | | | | _{M}G_{F}C_{M}U_{F}G_{M}U | | UCU | | | C_{M}A_{F}G_{M}C_{F}A | | CACC | |
| | | | | _{F}U_{M}C_{F}G_{M}U_{F}^{S}C | | | | | _{M}C_{F}C_{M}A_{F}G_{M}C_{F} | | | |
| | | | | _{M}U_{F}-OH-3′ | | | | | A_{M}^{S}C_{M}^{S}C_{M}- | | | |
| | | | | | | | | | OH-3′ | | | |
| DT- | 298 | 318 | DTS- | 5′-OH- | 803 | GGCAAU | 1065 | DTS- | 5′-PO4- | 921 | AUCAGU | 1140 |
| 001287 | | | 001947 | G_{F}^{S}G_{M}^{S}C_{F}A_{M}A | | GGACAC | | 001948 | A_{M}^{S}U_{F}^{S}C_{M}A_{F}G | | UGCGUG | |
| | | | | _{F}U_{M}G_{F}G_{M}A_{F}C | | GCAACU | | | _{M}U_{F}U_{M}G_{F}C_{M}G | | UCCAUU | |
| | | | | _{M}A_{F}C_{M}G_{F}C_{M}A | | GAU | | | _{F}U_{M}G_{F}U_{M}C_{F}C | | GCCCA | |
| | | | | _{F}A_{M}C_{F}U_{M}G_{F}A | | | | | _{M}A_{F}U_{M}U_{F}G_{M}C | | | |
| | | | | _{M}U_{F}-OH-3′ | | | | | C_{M}^{S}C_{M}^{S}A_{M}- | | | |
| | | | | | | | | | OH-3′ | | | |
| DT- | 302 | 322 | DTS- | 5′-OH- | 804 | AUGGAC | 1001 | DTS- | 5′-PO4- | 922 | AGAGAU | 1129 |
| 001288 | | | 001949 | A_{F}^{S}U_{M}^{S}G_{F}G_{M}A | | ACGCAAC | | 001950 | A_{M}^{S}G_{F}^{S}A_{M}G_{F}A | | CAGUUG | |
| | | | | _{F}C_{M}A_{F}C_{M}G_{F}C_{M} | | UGAUCU | | | _{M}U_{F}C_{M}A_{F}G_{M}U | | CGUGUCC | |
| | | | | A_{F}A_{M}C_{F}U_{M}G_{F} | | CU | | | _{F}U_{M}G_{F}C_{M}G_{F}U | | AUUC | |
| | | | | A_{M}U_{F}C_{M}U_{F}^{S}C | | | | | _{M}G_{F}U_{M}C_{F}C_{M}A | | | |
| | | | | _{M}U_{F}-OH-3′ | | | | | _{F}U_{M}^{S}U_{M}^{S}C_{M}- | | | |
| | | | | | | | | | OH-3′ | | | |
| DT- | 348 | 368 | DTS- | 5′-OH- | 805 | AGGAAA | 994 | DTS- | 5′-PO4- | 923 | AAACAG | 1112 |
| 001289 | | | 001951 | A_{F}^{S}G_{M}^{S}G_{F}A_{M}A | | UGUCCAC | | 001952 | A_{M}^{S}A_{F}^{S}A_{M}C_{F}A | | UGGUGG | |
| | | | | _{F}A_{M}U_{F}G_{M}U_{F}C | | CACUGU | | | _{M}G_{F}U_{M}G_{F}G_{M}U | | ACAUUU | |
| | | | | _{M}C_{F}A_{M}C_{F}C_{M}A_{F} | | UU | | | _{F}G_{M}G_{F}A_{M}C_{F}A | | CCUGA | |
| | | | | C_{M}U_{F}G_{M}U_{F}U_{M} | | | | | _{M}U_{F}U_{M}U_{F}C_{M}C | | | |
| | | | | U_{F}-OH-3′ | | | | | _{F}U_{M}^{S}G_{M}^{S}A_{M}- | | | |
| | | | | | | | | | OH-3′ | | | |
| DT- | 356 | 376 | DTS- | 5′-OH- | 806 | UCCACCA | 1086 | DTS- | 5′-PO4- | 924 | AUGAUG | 1145 |
| 001290 | | | 001953 | U_{F}^{S}C_{M}^{S}C_{F}A_{M}C | | CUGUUU | | 001954 | A_{M}^{S}U_{F}^{S}G_{M}A_{F}U | | AGAAAC | |
| | | | | _{F}C_{M}A_{F}C_{M}U_{F}G | | CUCAUCA | | | _{M}G_{F}A_{M}G_{F}A_{M}A | | AGUGGU | |
| | | | | _{M}U_{F}U_{M}U_{F}C_{M}U | | U | | | _{F}A_{M}C_{F}A_{M}G_{F}U | | GGACA | |
| | | | | _{F}C_{M}A_{F}U_{M}C_{F}^{S}A | | | | | _{M}G_{F}G_{M}U_{F}G_{M}G | | | |
| | | | | _{M}^{S}U_{F}-OH-3′ | | | | | _{F}A_{M}^{S}C_{M}^{S}A_{M}- | | | |
| | | | | | | | | | OH-3′ | | | |
| DT- | 381 | 401 | DTS- | 5′-OH- | 807 | AAACGA | 977 | DTS- | 5′-PO4- | 925 | ACAGAC | 1125 |
| 001291 | | | 001955 | A_{F}^{S}A_{M}^{S}A_{F}C_{M}G | | AUGGCU | | 001956 | A_{M}^{S}C_{F}^{S}A_{M}G_{F}A | | UGCAGCC | |
| | | | | _{F}A_{M}A_{F}U_{M}G_{F}G | | GCAGUC | | | _{M}C_{F}U_{M}G_{F}C_{M}A | | AUUCGU | |
| | | | | _{M}C_{F}U_{M}G_{F}C_{M}A | | UGU | | | _{F}G_{M}C_{F}C_{M}A_{F}U | | UUGG | |
| | | | | _{F}G_{M}U_{F}C_{M}U_{F}^{S}G | | | | | _{M}U_{F}C_{M}G_{F}U_{M}U | | | |
| | | | | _{M}^{S}U_{F}-OH-3′ | | | | | _{F}U_{M}^{S}G_{M}^{S}G_{M}- | | | |
| | | | | | | | | | OH-3′ | | | |
| DT- | 405 | 425 | DTS- | 5′-OH- | 808 | GGCCACC | 1067 | DTS- | 5′-PO4- | 926 | AUCGAC | 1141 |
| 001292 | | | 001957 | G_{F}^{S}G_{M}^{S}C_{F}C_{M}A | | AUGAUC | | 001958 | A_{M}^{S}U_{F}^{S}C_{M}G_{F}A | | AGGAUC | |
| | | | | _{F}C_{M}C_{F}A_{M}U_{F}G | | CUGUCG | | | _{M}C_{F}A_{M}G_{F}G_{M}A | | AUGGUG | |
| | | | | _{M}A_{F}U_{M}C_{F}C_{M}U | | AU | | | _{F}U_{M}C_{F}A_{M}U_{F}G | | | |
| | | | | _{F}G_{M}U_{F}C_{M}G_{F}^{S}A | | | | | _{M}G_{F}U_{M}G_{F}G_{M}C | | | |
| | | | | _{M}U_{F}-OH-3′ | | | | | _{F}C_{M}^{S}U_{M}G_{M}- | | | |
| | | | | | | | | | OH-3′ | | GCCUG | |
| DT- | 417 | 437 | DTS- | 5′-OH- | 809 | CCUGUCG | 1021 | DTS- | 5′-PO4- | 927 | AUGCUG | 1147 |
| 001293 | | | 001959 | C_{F}^{S}C_{M}^{S}U_{F}G_{M}U | | AUCAUC | | 001960 | A_{M}^{S}U_{F}^{S}G_{M}C_{F}U | | AAGAUG | |
| | | | | _{F}C_{M}G_{F}A_{M}U_{F}C | | UUCAGC | | | _{M}G_{F}A_{M}A_{F}G_{M}A | | AUCGAC | |
| | | | | _{M}A_{F}U_{M}C_{F}U_{M}U | | AU | | | _{F}U_{M}G_{F}A_{M}U_{F}C | | AGGAU | |
| | | | | _{F}C_{M}A_{F}G_{M}C_{F}^{S}A | | | | | _{M}G_{F}A_{M}C_{F}A_{M}G | | | |
| | | | | _{M}U_{F}-OH-3′ | | | | | _{F}G_{M}^{S}A_{M}^{S}U_{M}- | | | |
| | | | | | | | | | OH-3′ | | | |
| DT- | 447 | 467 | DTS- | 5′-OH- | 810 | GUUCCU | 1077 | DTS- | 5′-PO4- | 928 | AGUUGG | 1134 |
| 001294 | | | 001961 | G_{F}^{S}U_{M}^{S}U_{F}C_{M}C | | GUUCUU | | 001962 | A_{M}^{S}G_{F}^{S}U_{M}U_{F}G | | CAGAAG | |
| | | | | _{F}U_{M}G_{F}U_{M}U_{F}C | | CUGCCAA | | | _{M}G_{F}C_{M}A_{F}G_{M}A | | AACAGG | |
| | | | | _{M}U_{F}U_{M}C_{F}U_{M}G | | CU | | | _{F}A_{M}G_{F}A_{M}A_{F}C | | AACAG | |
| | | | | _{F}C_{M}C_{F}A_{M}A_{F}^{S}C | | | | | _{M}A_{F}G_{M}G_{F}A_{M}A | | | |
| | | | | _{M}^{S}U_{F}-OH-3′ | | | | | C_{M}^{S}A_{M}^{S}G_{M}- | | | |
| | | | | | | | | | OH-3′ | | | |
| DT- | 450 | 470 | DTS- | 5′-OH- | 811 | CCUGUUC | 1022 | DTS- | 5′-PO4- | 929 | AAGAGU | 1117 |
| 001295 | | | 001963 | C_{F}^{S}C_{M}^{S}U_{F}G_{M}U | | UUCUGCC | | 001964 | A_{M}^{S}A_{F}^{S}G_{M}A_{F}G | | UGGCAG | |
| | | | | _{F}U_{M}C_{F}U_{M}U_{F}C | | AACUCU | | | _{M}U_{F}U_{M}G_{F}G_{M}C | | AAGAAC | |
| | | | | _{M}U_{F}G_{M}C_{F}C_{M}A | | U | | | _{F}A_{M}G_{F}A_{M}A_{F}G | | AGGAA | |
| | | | | _{F}A_{M}C_{F}U_{M}CE^{S}U | | | | | _{M}A_{F}A_{M}C_{F}A_{M}G | | | |
| | | | | _{M}U_{F}-OH-3′ | | | | | _{F}G_{M}^{S}A_{M}^{S}A_{M}- | | | |
| | | | | | | | | | OH-3′ | | | |
| DT- | 462 | 482 | DTS- | 5′-OH- | 821 | CCAACUC | 1010 | DTS- | 5′-PO4- | 930 | UUGGUG | 1165 |
| 001308 | | | 001974 | C_{F}^{S}C_{M}^{S}A_{F}A_{M}C | | UUCACCC | | 001975 | U_{M}^{S}U_{F}^{S}G_{M}G_{F}U | | AGGGUG | |
| | | | | U_{M}C_{F}U_{M}U_{F}C | | UCACCAA | | | _{M}G_{F}A_{M}G_{F}G_{M}G | | AAGAGU | |
| | | | | _{M}A_{F}C_{M}C_{F}C_{M}U_{F} | | | | | _{F}U_{M}G_{F}A_{M}A_{F}G | | UGGCA | |
| | | | | C_{M}A_{F}C_{M}C_{F}^{S}A_{M} | | | | | _{M}A_{F}G_{M}U_{F}U_{M}G | | | |
| | | | | ^{S}A_{F}-OH-3′ | | | | | _{F}G_{M}^{S}C_{M}^{S}A_{M}- | | | |
| | | | | | | | | | OH-3′ | | | |
| DT- | 484 | 504 | DTS- | 5′-OH- | 822 | GGGGGC | 1071 | DTS- | 5′-PO4- | 931 | AGUGAU | 1133 |
| 001309 | | | 001976 | G_{F}^{S}G_{M}^{S}G_{F}G_{M}G | | AGGUUU | | 001977 | A_{M}^{S}G_{F}^{S}U_{M}G_{F}A | | GUAAAA | |
| | | | | _{F}C_{M}A_{F}G_{M}G_{F}U | | UACAUC | | | _{M}U_{F}G_{M}U_{F}A_{M}A | | CCUGCCC | |
| | | | | _{M}U_{F}U_{M}U_{F}A_{M}C | | | | | _{F}A_{M}A_{F}C_{M}C_{F}U | | CCCU | |
| | | | | _{F}A_{M}U_{F}C_{M}A_{F}^{S}C | | | | | _{M}G_{F}C_{M}C_{F}C_{M}C_{F} | | | |
| | | | | _{M}U_{F}-OH-3′ | | | | | C_{M}^{S}C_{M}^{S}U_{M}- | | | |
| | | | | | | ACU | | | OH-3′ | | | |
| DT- | 489 | 509 | DTS- | 5′-OH- | 823 | CAGGUU | 1009 | DTS- | 5′-PO4- | 932 | AUUCCA | 1150 |
| 001310 | | | 001978 | C_{F}^{S}A_{M}^{S}G_{F}G_{M}U | | UUACAU | | 001979 | A_{M}^{S}U_{F}^{S}U_{M}C_{F}C | | GUGAUG | |
| | | | | _{F}U_{M}U_{F}U_{M}A_{F}C | | CACUGG | | | _{M}A_{F}G_{M}U_{F}G_{M}A | | UAAAAC | |
| | | | | _{M}A_{F}U_{M}C_{F}A_{M}C | | AAU | | | _{F}U_{M}G_{F}U_{M}A_{F}A | | CUGCC | |
| | | | | _{F}U_{M}G_{F}G_{M}A_{F}^{S}A | | | | | _{M}A_{F}A_{M}C_{F}C_{M}U | | | |
| | | | | _{M}U_{F}-OH-3′ | | | | | _{F}G_{M}^{S}C_{M}^{S}C_{M}- | | | |
| | | | | | | | | | OH-3′ | | | |
| DT- | 492 | 512 | DTS- | 5′-OH- | 824 | GUUUUA | 1081 | DTS- | 5′-PO4- | 933 | AAGAUU | 1119 |
| 001311 | | | 001980 | G_{F}^{S}U_{M}^{S}U_{F}U_{M}U | | CAUCACU | | 001981 | A_{M}^{S}A_{F}^{S}G_{M}A_{F}U | | CCAGUG | |
| | | | | _{F}A_{M}C_{F}A_{M}U_{F}C | | GGAAUC | | | _{M}U_{F}C_{M}C_{F}A_{M}G | | AUGUAA | |
| | | | | _{M}A_{F}C_{M}U_{F}G_{M}G | | UU | | | _{F}U_{M}G_{F}A_{M}U_{F}G | | AACCU | |
| | | | | _{F}A_{M}A_{F}U_{M}CE^{S}U | | | | | _{M}U_{F}A_{M}A_{F}A_{M}A | | | |
| | | | | _{M}U_{F}-OH-3′ | | | | | C_{M}^{S}C_{M}^{S}U_{M}- | | | |
| | | | | | | | | | OH-3′ | | | |
| DT- | 499 | 519 | DTS- | 5′-OH- | 825 | AUCACU | 997 | DTS- | 5′-PO4- | 934 | AAUUUG | |
| 001312 | | | 001982 | A_{F}^{S}U_{M}^{S}C_{F}A_{M}C | | GGAAUC | | 001983 | A_{M}^{S}A_{F}^{S}U_{M}U_{F}U | | GAAGAU | |
| | | | | _{F}U_{M}G_{F}G_{M}A_{F}A | | UUCCAA | | | _{M}G_{F}G_{M}A_{F}A_{M}G | | UCCAGU | |
| | | | | _{M}U_{F}C_{M}U_{F}U_{M}C | | AUU | | | _{F}A_{M}U_{F}U_{M}C_{F}C | | GAUGU | |
| | | | | _{F}C_{M}A_{F}A_{M}A_{F}^{S}U | | | | | _{M}A_{F}G_{M}U_{F}G_{M}A | | | |
| | | | | _{M}U_{F}-OH-3′ | | | | | U_{M}^{S}G_{M}^{S}U_{M}- | | | |
| | | | | | | | | | OH-3′ | | | 1124 |
| DT- | 505 | 525 | DTS- | 5′-OH- | 826 | GGAAUC | 1063 | DTS- | 5′-PO4- | 935 | AGCAAG | 1130 |
| 001313 | | | 001984 | G_{F}^{S}G_{M}^{S}A_{F}A_{M}U | | UUCCAA | | 001985 | A_{M}^{S}G_{F}^{S}C_{M}A_{F}A | | AAUUUG | |
| | | | | C_{M}U_{F}U_{M}C_{F}C_{M} | | AUUCUU | | | _{M}G_{F}A_{M}A_{F}U_{M}U | | GAAGAU | |
| | | | | A_{F}A_{M}A_{F}U_{M}U_{F} | | GCU | | | _{F}U_{M}G_{F}G_{M}A_{F}A | | UCCAG | |
| | | | | C_{M}U_{F}U_{M}G_{F}^{S}C | | | | | _{M}G_{F}A_{M}U_{F}U_{M}C | | | |
| | | | | ^{S}U_{F}-OH-3′ | | | | | _{F}C_{M}^{S}A_{M}^{S}G_{M}- | | | |
| | | | | | | | | | OH-3′ | | | |
| DT- | 534 | 554 | DTS- | 5′-OH- | 827 | CGUGAU | 1029 | DTS- | 5′-PO4- | 936 | AUGGCC | 1148 |
| 001314 | | | 001986 | C_{F}^{S}G_{M}^{S}U_{F}G_{M}A | | GAGUGC | | 001987 | A_{M}^{S}U_{F}^{S}G_{M}G_{F}C | | GCAGCAC | |
| | | | | _{F}U_{M}G_{F}A_{M}G_{F}U | | UGCGGCC | | | _{M}C_{F}G_{M}C_{F}A_{M}G | | UCAUCAC | |
| | | | | _{M}G_{F}C_{M}U_{F}G_{M}C | | AU | | | C_{M}A_{F}C_{M}U_{F}C_{M} | | GCA | |
| | | | | _{F}G_{M}G_{F}C_{M}C_{F}^{S}A | | | | | A_{F}U_{M}C_{F}A_{M}C_{F} | | | |
| | | | | _{M}U_{F}-OH-3′ | | | | | G_{M}^{S}C_{M}^{S}A_{M}- | | | |
| | | | | | | | | | OH-3′ | | | |
| DT- | 567 | 587 | DTS- | 5′-OH- | 828 | GCACCCG | 1056 | DTS- | 5′-PO4- | 937 | UUGAGA | 1163 |
| 001315 | | | 001988 | G_{F}^{S}C_{M}^{S}A_{F}C_{M}C | | GAGUGG | | 001989 | U_{M}^{S}U_{F}^{S}G_{M}A_{F}G | | UGCCACU | |
| | | | | _{F}C_{M}G_{F}G_{M}A_{F}G | | CAUCUCA | | | _{M}A_{F}U_{M}G_{F}C_{M}C | | CCGGGU | |
| | | | | _{M}U_{F}G_{M}G_{F}C_{M}A | | A | | | _{F}A_{M}C_{F}U_{M}C_{F}C_{M} | | GCCU | |
| | | | | _{F}U_{M}C_{F}U_{M}C_{F}^{S}A | | | | | G_{F}G_{M}G_{F}U_{M}G_{F} | | | |
| | | | | _{M}A_{F}-OH-3′ | | | | | C_{M}^{S}C_{M}^{S}U_{M}- | | | |
| | | | | | | | | | OH-3′ | | | |
| DT- | 588 | 608 | DTS- | 5′-OH- | 829 | CUCGGA | 1039 | DTS- | 5′-PO4- | 938 | AAACCG | 1113 |
| 001316 | | | 001990 | C_{F}^{S}U_{M}^{S}C_{F}G_{M}G | | UUACUCC | | 001991 | A_{M}^{S}A_{F}^{S}A_{M}C_{F}C | | UAGGAG | |
| | | | | _{F}A_{M}U_{F}U_{M}A_{F}C | | UACGGU | | | _{M}G_{F}U_{M}A_{F}G_{M}G | | UAAUCC | |
| | | | | _{M}U_{F}C_{M}C_{F}U_{M}A | | UU | | | _{F}A_{M}G_{F}U_{M}A_{F}A | | GAGUU | |
| | | | | _{F}C_{M}G_{F}G_{M}U_{F}^{S}U | | | | | _{M}U_{F}C_{M}C_{F}G_{M}A | | | |
| | | | | _{M}U_{F}-OH-3′ | | | | | _{F}G_{M}^{S}U_{M}^{S}U_{M}- | | | |
| | | | | | | | | | OH-3′ | | | |
| DT- | 597 | 617 | DTS- | 5′-OH- | 830 | CUCCUAC | 1033 | DTS- | 5′-PO4- | 939 | AUGUAG | 1149 |
| 001317 | | | 001992 | C_{F}^{S}U_{M}^{S}C_{F}C_{M}U | | GGUUUC | | 001993 | A_{M}^{S}U_{F}^{S}G_{M}U_{F}A | | GCGAAA | |
| | | | | _{F}A_{M}C_{F}G_{M}G_{F}U | | GCCUACA | | | _{M}G_{F}G_{M}C_{F}G_{M}A | | CCGUAG | |
| | | | | _{M}U_{F}U_{M}C_{F}G_{M}C | | U | | | _{F}A_{M}A_{F}C_{M}C_{F}G | | GAGUA | |
| | | | | C_{M}U_{F}A_{M}CE^{S}A | | | | | _{M}U_{F}A_{M}G_{F}G_{M}A | | | |
| | | | | _{M}^{S}U_{F}-OH-3′ | | | | | _{F}G_{M}^{S}U_{M}^{S}A_{M}- | | | |
| | | | | | | | | | OH-3′ | | | |
| DT- | 600 | 620 | DTS- | 5′-OH- | 831 | CUACGG | 1031 | DTS- | 5′-PO4- | 940 | AGGAUG | 1132 |
| 001318 | | | 001994 | C_{F}^{S}U_{M}^{S}A_{F}C_{M}G | | UUUCGCC | | 001995 | A_{M}^{S}G.^{S}G_{M}A_{F}U | | UAGGCG | |
| | | | | _{F}G_{M}U_{F}U_{M}U_{F}C | | UACAUCC | | | _{M}G_{F}U_{M}A_{F}G_{M}G | | AAACCG | |
| | | | | _{M}G_{F}C_{M}C_{F}U_{M}A | | U | | | _{F}C_{M}G_{F}A_{M}A_{F}A | | UAGGA | |
| | | | | _{F}C_{M}A_{F}U_{M}C_{F}^{S}C | | | | | _{M}C_{F}C_{M}G_{F}U_{M}A | | | |
| | | | | _{M}U_{F}-OH-3′ | | | | | _{F}G_{M}G_{M}^{S}A_{M}- | | | |
| | | | | | | | | | OH-3′ | | | |
| DT- | 651 | 671 | DTS- | 5′-OH- | 832 | CAGCGG | 1008 | DTS- | 5′-PO4- | 941 | AUCACA | 1139 |
| 001319 | | | 001996 | C_{F}^{S}A_{M}^{S}G_{F}C_{M}G | | UGUCAU | | 001997 | A_{M}^{S}U_{F}^{S}C_{M}A_{F}C | | UAGAUG | |
| | | | | _{F}G_{M}U_{F}G_{M}U_{F}C | | CUAUGU | | | _{M}A_{F}U_{M}A_{F}G_{M}A | | ACACCGC | |
| | | | | _{M}A_{F}U_{M}C_{F}U_{M}A | | GAU | | | _{F}U_{M}G_{F}A_{M}C_{F}A | | UGAG | |
| | | | | _{F}U_{M}G_{F}U_{M}GE^{S}A | | | | | _{M}C_{F}C_{M}G_{F}C_{M}U_{F} | | | |
| | | | | _{M}^{S}U_{F}-OH-3′ | | | | | OH-3′ | | | |
| DT- | 654 | 674 | DTS- | 5′-OH- | 833 | CGGUGU | 1026 | DTS- | 5′-PO4- | 942 | AAGAUC | 1118 |
| 001320 | | | 001998 | C_{F}^{S}G_{M}^{S}G_{F}U_{M}G | | CAUCUA | | 001999 | A_{M}^{S}A_{F}^{S}G_{M}A_{F}U | | ACAUAG | |
| | | | | _{F}U_{M}C_{F}A_{M}U_{F}C | | UGUGAU | | | _{M}C_{F}A_{M}C_{F}A_{M}U | | AUGACA | |
| | | | | _{M}U_{F}A_{M}U_{F}G_{M}U | | CUU | | | _{F}A_{M}G_{F}A_{M}U_{F}G | | CCGCU | |
| | | | | _{F}G_{M}A_{F}U_{M}C_{F}^{S}U | | | | | _{M}A_{F}C_{M}A_{F}C_{M}C_{F} | | | |
| | | | | _{M}U_{F}-OH-3′ | | | | | G_{M}^{S}C_{M}^{S}U_{M}- | | | |
| | | | | | | | | | OH-3′ | | | |
| DT- | 661 | 681 | DTS- | 5′-OH- | 834 | AUCUAU | 999 | DTS- | 5′-PO4- | 943 | UUUCCGC | 1166 |
| 001321 | | | 002000 | A_{F}^{S}U_{M}^{S}C_{F}U_{M}A | | GUGAUC | | 002001 | U_{M}^{S}U_{F}^{S}U_{M}C_{F}C | | AAGAUC | |
| | | | | _{F}U_{M}G_{F}U_{M}G_{F}A | | UUGCGG | | | _{M}G_{F}C_{M}A_{F}A_{M}G | | ACAUAG | |
| | | | | _{M}U_{F}C_{M}U_{F}U_{M}G | | AAA | | | _{F}A_{M}U_{F}C_{M}A_{F}C | | AUGA | |
| | | | | _{F}C_{M}G_{F}G_{M}A_{F}^{S}A | | | | | _{M}A_{F}U_{M}A_{F}G_{M}A | | | |
| | | | | _{M}A_{F}-OH-3′ | | | | | _{F}U_{M}^{S}G_{M}^{S}A_{M}- | | | |
| | | | | | | | | | OH-3′ | | | |
| DT- | 783 | 803 | DTS- | 5′-OH- | 849 | AAAUCCC | 979 | DTS- | 5′-PO4- | 944 | UUUGGU | 1169 |
| 001336 | | | 002016 | A_{F}^{S}A_{M}^{S}A_{F}U_{M}C | | AAACUC | | 002017 | U_{M}^{S}U_{F}^{S}U_{M}G_{F}G | | UUGAGU | |
| | | | | _{F}C_{M}C_{F}A_{M}A_{F}A | | AAACCA | | | _{M}U_{F}U_{M}U_{F}G_{M}A | | UUGGGA | |
| | | | | _{M}C_{F}U_{M}C_{F}A_{M}A | | AA | | | _{F}G_{M}U_{F}U_{M}U_{F}G | | UUUUG | |
| | | | | _{F}A_{M}C_{F}C_{M}A_{F}^{S}A | | | | | _{M}G_{F}G_{M}A_{F}U_{M}U | | | |
| | | | | _{M}A_{F}-OH-3′ | | | | | _{F}U_{M}^{S}U_{M}^{S}G_{M}- | | | |
| | | | | | | | | | OH-3′ | | | |
| DT- | 832 | 852 | DTS- | 5′-OH- | 850 | UGCUGU | 1098 | DTS- | 5′-PO4- | 945 | AUACAU | 1137 |
| 001337 | | | 002018 | U_{F}^{S}G_{M}^{S}C_{F}U_{M}G | | UGAUUG | | 002019 | A_{M}^{S}U_{F}^{S}A_{M}C_{F}A | | CUUCAA | |
| | | | | _{F}U_{M}U_{F}G_{M}A_{F}U | | AAGAUG | | | _{M}U_{F}C_{M}U_{F}U_{M}C | | UCAACA | |
| | | | | _{M}U_{F}G_{M}A_{F}A_{M}G | | UAU | | | _{F}A_{M}A_{F}U_{M}C_{F}A | | | |
| | | | | _{F}A_{M}U_{F}G_{M}U_{F}^{S}A | | | | | _{M}A_{F}C_{M}A_{F}G_{M}C | | | |
| | | | | _{M}U_{F}-OH-3′ | | | | | _{F}A_{M}^{S}A_{M}^{S}C_{M}- | | | |
| | | | | | | | | | OH-3′ | | GCAAC | |
| DT- | 863 | 883 | DTS- | 5′-OH- | 851 | CGGUUU | 1027 | DTS- | 5′-PO4- | 946 | AUAAAU | 1135 |
| 001338 | | | 002020 | C_{F}^{S}G_{M}^{S}G_{F}U_{M}U | | AUAAAA | | 002021 | A_{M}^{S}U_{F}^{S}A_{M}A_{F}A | | AGGUUU | |
| | | | | _{F}U_{M}A_{F}U_{M}A_{F}A | | CCUAUU | | | _{M}U_{F}A_{M}G_{F}G_{M}U | | UAUAAA | |
| | | | | _{M}A_{F}A_{M}C_{F}C_{M}U | | UAU | | | _{F}U_{M}U_{F}U_{M}A_{F}U | | CCGGA | |
| | | | | _{F}A_{M}U_{F}U_{M}U_{F}^{S}A | | | | | _{M}A_{F}A_{M}A_{F}C_{M}C | | | |
| | | | | _{M}®^{S}U_{F}-OH-3′ | | | | | _{F}G_{M}G_{M}^{S}A_{M}- | | | |
| | | | | | | | | | OH-3′ | | | |
| DT- | 902 | 922 | DTS- | 5′-OH- | 852 | GUACAU | 1073 | DTS- | 5′-PO4- | 947 | AAAGCA | 1114 |
| 001339 | | | 002022 | G_{F}^{S}U_{M}^{S}A_{F}C_{M}A | | AGUAUU | | 002023 | A_{M}^{S}A_{F}^{S}A_{M}G_{F}C | | AACAAU | |
| | | | | _{F}U_{M}A_{F}G_{M}U_{F}A | | GUUUGC | | | _{M}A_{F}A_{M}A_{F}C_{M}A | | ACUAUG | |
| | | | | _{M}U_{F}U_{M}G_{F}U_{M}U | | UUU | | | _{F}A_{M}U_{F}A_{M}C_{F}U | | UACAU | |
| | | | | _{F}U_{M}G_{F}C_{M}U_{F}^{S}U | | | | | _{M}A_{F}U_{M}G_{F}U_{M}A | | | |
| | | | | ^{S}U_{F}-OH-3′ | | | | | _{F}C_{M}^{S}A_{M}^{S}U_{M}- | | | |
| | | | | | | | | | OH-3′ | | | |
| DT- | 927 | 947 | DTS- | 5′-OH- | 853 | GUUGAC | 1078 | DTS- | 5′-PO4- | 948 | AACACG | 1116 |
| 001340 | | | 002024 | G_{F}^{S}U_{M}^{S}U_{F}G_{M}A | | CAUCAGC | | 002025 | A_{M}^{S}A_{F}^{S}C_{M}A_{F}C | | AGGCUG | |
| | | | | _{F}C_{M}C_{F}A_{M}U_{F}C_{M} | | CUCGUG | | | _{M}G_{F}A_{M}G_{F}G_{M}C | | AUGGUC | |
| | | | | A_{F}G_{M}C_{F}C_{M}U_{F} | | UU | | | _{F}U_{M}G_{F}A_{M}U_{F}G | | AACAU | |
| | | | | C_{M}G_{F}U_{M}G_{F}^{S}U | | | | | _{M}G_{F}U_{M}C_{F}A_{M}A | | | |
| | | | | _{M}U_{F}-OH-3′ | | | | | C_{M}^{S}A_{M}^{S}U_{M}- | | | |
| | | | | | | | | | OH-3′ | | | |
| DT- | 956 | 976 | DTS- | 5′-OH- | 854 | AAGAAG | 981 | DTS- | 5′-PO4- | 949 | AAAGUU | 1115 |
| 001341 | | | 002026 | A_{F}^{S}A_{M}^{S}G_{F}A_{M}A | | UAGCUA | | 002027 | A_{M}^{S}A_{F}^{S}A_{M}G_{F}U | | CCUUAGC | |
| | | | | G_{M}U_{F}A_{M}G_{F}C | | AGGAAC | | | _{M}U_{F}C_{M}C_{F}U_{M}U | | UACUUC | |
| | | | | _{M}U_{F}A_{M}A_{F}G_{M}G | | UUU | | | _{F}A_{M}G_{F}C_{M}U_{F}A | | UUUA | |
| | | | | A_{M}A_{F}C_{M}U_{F}^{S}U | | | | | _{M}C_{F}U_{M}U_{F}C_{M}U | | | |
| | | | | _{M}^{S}U_{F}-OH-3′ | | | | | _{F}U_{M}^{S}U_{M}^{S}A_{M}- | | | |
| | | | | | | | | | OH-3′ | | | |
| DT- | 965 | 985 | DTS- | 5′-OH- | 855 | CUAAGG | 1030 | DTS- | 5′-PO4- | 950 | UUAGGA | 1159 |
| 001342 | | | 002028 | C_{F}^{S}U_{M}^{S}A_{F}A_{M}G | | AACUUU | | 002029 | U_{M}^{S}U_{F}^{S}A_{M}G_{F}G | | UGUAAA | |
| | | | | _{F}G_{M}A_{F}A_{M}C_{F}U | | ACAUCCU | | | _{M}A_{F}U_{M}G_{F}U_{M}A | | GUUCCU | |
| | | | | _{M}U_{F}U_{M}A_{F}C_{M}A | | AA | | | _{F}A_{M}A_{F}G_{M}U_{F}U | | UAGCU | |
| | | | | _{F}U_{M}C_{F}C_{M}U_{F}^{S}A | | | | | _{M}C_{F}C_{M}U_{F}U_{M}A | | | |
| | | | | _{M}^{S}A_{F}-OH-3′ | | | | | _{F}G_{M}^{S}C_{M}^{S}U_{M}- | | | |
| | | | | | | | | | OH-3′ | | | |
| DT- | 1784 | 1804 | DTS- | 5′-OH- | 856 | ACUGUG | 992 | DTS- | 5′-PO4- | 951 | AUGCAU | 1146 |
| 001343 | | | 002030 | A_{F}^{S}C_{M}^{S}U_{F}G_{M}U | | UGGACU | | 002031 | A_{M}^{S}U_{F}^{S}G_{M}C_{F}A | | CUUAGU | |
| | | | | _{F}G_{M}U_{F}G_{M}G_{F}A | | AAGAUG | | | _{M}U_{F}C_{M}U_{F}U_{M}A | | CCACACA | |
| | | | | _{M}C_{F}U_{M}A_{F}A_{M}G | | CAU | | | _{F}G_{M}U_{F}C_{M}C_{F}A | | GUUG | |
| | | | | _{F}A_{M}U_{F}G_{M}C_{F}^{S}A | | | | | _{M}C_{F}A_{M}C_{F}A_{M}G | | | |
| | | | | _{M}^{S}U_{F}-OH-3′ | | | | | _{F}U_{M}^{S}U_{M}^{S}G_{M}- | | | |
| | | | | | | | | | OH-3′ | | | |
| DT- | 160 | 180 | DTS- | 5′-OH- | 865 | CGCUGU | 1024 | DTS- | 5′-PO4- | 952 | UUUCUG | 1167 |
| 001352 | | | 002040 | C_{F}^{S}G_{M}C_{F}U_{M}G | | UUGGCC | | 002041 | U_{M}^{S}U_{F}^{S}U_{M}C_{F}U | | CCCGGCC | |
| | | | | _{F}U_{M}U_{F}U_{M}G_{F}G | | GGGCAG | | | _{M}G_{F}C_{M}C_{F}C_{M}G_{F} | | AAACAG | |
| | | | | _{M}C_{F}C_{M}G_{F}G_{M}G | | AAA | | | G_{M}C_{F}C_{M}A_{F}A_{M} | | CGUA | |
| | | | | C_{M}A_{F}G_{M}A_{F}^{S}A | | | | | A_{F}C_{M}A_{F}G_{M}C_{F} | | | |
| | | | | _{M}A_{F}-OH-3′ | | | | | G_{M}^{S}U_{M}^{S}A_{M}- | | | |
| | | | | | | | | | OH-3′ | | | |
| DT- | 175 | 195 | DTS- | 5′-OH- | 866 | CAGAAA | 1007 | DTS- | 5′-PO4- | 953 | UUCUGC | 1162 |
| 001353 | | | 002042 | C_{F}^{S}A_{M}^{S}G_{F}A_{M}A | | CUCCGCU | | 002043 | U_{M}^{S}U_{F}^{S}C_{M}U_{F}G | | UCAGCG | |
| | | | | _{F}A_{M}C_{F}U_{M}C_{F}C_{M} | | GAGCAG | | | _{M}C_{F}U_{M}C_{F}A_{M}G | | GAGUUU | |
| | | | | G_{F}C_{M}U_{F}G_{M}A_{F} | | AA | | | _{F}C_{M}G_{F}G_{M}A_{F}G | | CUGCC | |
| | | | | G_{M}C_{F}A_{M}GE^{S}A | | | | | _{M}U_{F}U_{M}U_{F}C_{M}U | | | |
| | | | | _{M}A_{F}-OH-3′ | | | | | G_{M}^{S}C_{M}^{S}C_{M}- | | | |
| | | | | | | | | | OH-3′ | | | |
| DT- | 178 | 198 | DTS- | 5′-OH- | 867 | AAACUCC | 978 | DTS- | 5′-PO4- | 954 | AAGUUC | 1120 |
| 001354 | | | 002044 | A_{F}^{S}A_{M}^{S}A_{F}C_{M}U | | GCUGAG | | 002045 | A_{M}^{S}A_{F}^{S}G_{M}U_{F}U | | UGCUCA | |
| | | | | _{F}C_{M}C_{F}G_{M}C_{F}U_{M} | | CAGAAC | | | _{M}C_{F}U_{M}G_{F}C_{M}U | | GCGGAG | |
| | | | | G_{F}A_{M}G_{F}C_{M}A_{F} | | UU | | | C_{M}A_{F}G_{M}C_{F}G | | UUUCU | |
| | | | | G_{M}A_{F}A_{M}C_{F}^{S}U | | | | | _{M}G_{F}A_{M}G_{F}U_{M}U | | | |
| | | | | _{M}U_{F}-OH-3′ | | | | | U_{M}^{S}C_{M}^{S}U_{M}- | | | |
| | | | | | | | | | OH-3′ | | | |

### Example 4: Conjugated siRNAs Targeting PMP22

The 3′ terminus of the sense strand of certain compounds was conjugated to a long chain fatty acid (LCFA) domain or “uptake motif” which improves the uptake of nucleic acid compounds into cells both in vitro and in vivo (International Patent Application Publication No. WO 2019/232255). The conjugated compounds are shown in Table 4. “Start” and “End” correspond to the 5′ and 3′ nucleotide positions of the nucleotide sequence of the human PMP22 mRNA (NCBI Reference Sequence NM_000304.4, deposited with GenBank on Nov. 22, 2018; SEQ ID NO: 1170) to which the nucleotides of the antisense strand are complementary. Each row represents a sense and antisense strand pair of an siRNA. The nucleotide sequences for both the modified and unmodified sense and antisense strands are included.

Conjugated compounds were formed as in the structures below, where the nucleotide shown is the 3′ terminal nucleotide, “B” is nucleobase and “R” is the substituent at the 2′ carbon of the nucleoside sugar.

The uptake motif DTx-01-08 was conjugated to the sense strand, using the “C7OH” linker

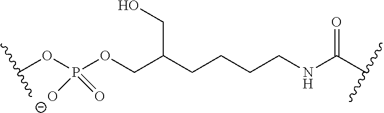 [SMILES: *OP(=O)([O-])OCC(CO)CCCCNC(*)=O]

attached to the 3′ carbon of the 3′ terminal nucleotide of the sense strand via the phosphate group to form the conjugate group named “C7OH-[DTx-01-08] in Table 4.

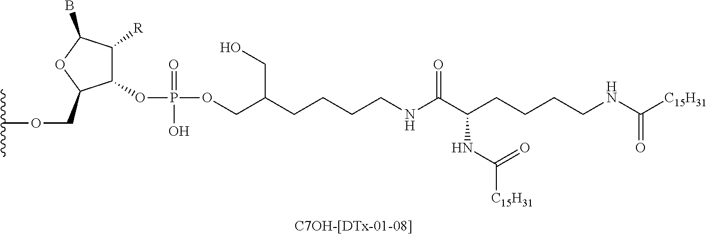

The uptake motif DTx-01-32 was conjugated to the sense strand, using the “C7OH” linker

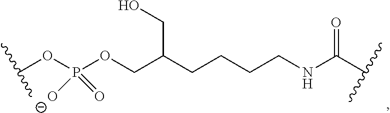 [SMILES: *OP(=O)([O-])OCC(CO)CCCCNC(*)=O]

attached to the 3′ carbon of the 3′ terminal nucleotide of the sense strand via the phosphate group to form the conjugate group named “C7OH-[DTx-01-32] in Table 4.

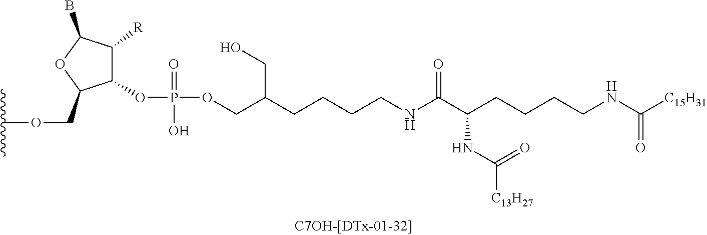

In Table 4 and elsewhere herein, modified sugar moieties are indicated by a subscript notation following the nucleotide, and modified internucleotide linkages are indicated by a superscript notation. 5′ and 3′ terminal groups are also indicated. A nucleotide followed by the subscript “F” is a 2′-fluoro nucleotide; a nucleotide followed by the subscript “M” is a 2′-O-methyl nucleotide; a nucleotide followed by the subscript “E” is a 2′-O-methoxyethyl nucleotide; and a nucleotide followed by the subscript “D” is a beta-D-deoxyribonucleotide. The nucleobase of each “C_{E}” nucleotide is a 5-methylcytosine; each other “C” is a non-methylated cytosine; the nucleobase of each “UE” nucleotide is a 5-methyluracil; each other “U” is a non-methylated uridine. A superscript “S” is a phosphorothioate internucleotide linkage; all other internucleotide linkages are phosphodiester internucleotide linkages. For example, “U_{F}^{S}C_{M}” is a 2′-fluorouridine linked to a 2′-O-methylcytidine by a phosphorothioate internucleotide linkage. “G_{M}U_{F}” is a 2-O-methylguanosine linked to a 2′-fluorouridine by a phosphodiester internucleotide linkage. A hydroxyl group is at the 5′ carbon of the 5′ terminal nucleotide is indicated by “5′-OH”; a phosphate group at the 5′ carbon of the 5′ terminal nucleotide is indicated by “5′-PO4”; a 5′-VP modification at the 5′ terminal nucleotide of an antisense strand is indicated by “5′-VP”; and a hydroxyl group at the 3′ carbon of the 3′ terminal nucleotide is indicated by “OH-3′.”

TABLE 4
Conjugated SiRNAs targeting PMP22

| | | | | Modified | SEQ | Unmodified | SEQ | | Modified | SEQ | Unmodified | SEQ |
|---|---|---|---|---|---|---|---|---|---|---|---|---|
| siRNA | | | Strand | Nucleotide | ID | Nucleotide | ID | Strand | Nucleotide | ID | Nucleotide | ID |
| ID | Start | End | ID | Sequence | NO | Sequence | NO | ID | Sequence | NO | Sequence | NO |
| DT- | 316 | 334 | DTS- | 5′-OH- | 646 | GAUCUC | 1055 | DTS- | 5′-PO4- | 167 | UACAGU | 590 |
| 000544 | | | 000851 | G_{F}^{S}A_{M}^{S}U_{F}C_{M}U_{F}C | | UGGCAG | | 000599 | U_{M}^{S}A_{F}^{S}C_{M}A_{F}G_{M} | | UCUGCCA | |
| | | | | _{M}U_{F}G_{M}G_{F}C_{M}A_{F}G | | AACUGU | | | U_{F}U_{M}C_{F}U_{M}G_{F}C | | GAGAUCT | |
| | | | | _{M}A_{F}A_{M}C_{F}U_{M}G_{F}^{S}U | | A | | | _{M}C_{F}A_{M}G_{F}A_{M}G_{F} | | T | |
| | | | | _{M}^{S}A_{F}-C7OH- | | | | | A_{M}U_{F}C_{M}^{S}T_{D}^{S}T_{D}- | | | |
| | | | | [_{D}Tx-01-08] | | | | | OH-3′ | | | |
| DT- | 443 | 461 | DTS- | 5′-OH- | 647 | CUCUGU | 1040 | DTS- | 5′-PO4- | 172 | CAGAAG | 576 |
| 000545 | | | 000852 | C_{F}^{S}U_{M}^{S}C_{F}U_{M}G_{F}U | | UCCUGU | | 000609 | C_{M}^{S}A_{F}^{S}G_{M}A_{F}A_{M} | | AACAGG | |
| | | | | _{M}U_{F}C_{M}C_{F}U_{M}G_{F}U | | UCUUCU | | | G_{F}A_{M}A_{F}C_{M}A_{F}G | | AACAGA | |
| | | | | _{M}U_{F}C_{M}U_{F}U_{M}C_{F}^{S}U | | G | | | _{M}G_{F}A_{M}A_{F}C_{M}A_{F} | | GTT | |
| | | | | _{M}^{S}G_{F}-C7OH- | | | | | G_{M}A_{F}G_{M}^{S}T_{D}^{S}T_{D}- | | | |
| | | | | [_{D}Tx-01-08] | | | | | OH-3′ | | | |
| DT- | 521 | 539 | DTS- | 5′-OH- | 648 | UUGCUG | 1107 | DTS- | 5′-PO4- | 174 | AUCACGC | 546 |
| 000546 | | | 000853 | U_{F}^{S}U_{M}^{S}G_{F}C_{M}U_{F}G | | GUCUGU | | 000613 | A_{M}^{S}U_{F}^{S}C_{M}A_{F}C_{M} | | ACAGACC | |
| | | | | _{M}G_{F}U_{M}C_{F}U_{M}G_{F}U | | GCGUGA | | | G_{F}C_{M}A_{F}C_{M}A_{F}G | | AGCAATT | |
| | | | | _{M}G_{F}C_{M}G_{F}U_{M}G_{F}^{S}A | | U | | | _{M}A_{F}C_{M}C_{F}A_{M}G_{F} | | | |
| | | | | _{M}^{S}U_{F}-C7OH- | | | | | C_{M}A_{F}A_{M}^{S}T_{D}^{S}T_{D}- | | | |
| | | | | [_{D}Tx-01-08] | | | | | OH-3′ | | | |
| DT- | 443 | 461 | DTS- | 5′-OH- | 649 | CUCUGU | 1040 | DTS- | 5′-PO4- | 172 | CAGAAG | 576 |
| 000620 | | | 000950 | C_{F}^{S}U_{M}^{S}C_{F}U_{M}G_{F}U | | UCCUGU | | 000609 | C_{M}^{S}A_{F}^{S}G_{M}A_{F}A_{M} | | AACAGG | |
| | | | | _{M}U_{F}C_{M}C_{F}U_{M}G_{F}U | | UCUUCU | | | G_{F}A_{M}A_{F}C_{M}A_{F}G | | AACAGA | |
| | | | | _{M}U_{F}C_{M}U_{F}U_{M}C_{F}^{S}U | | G | | | _{M}G_{F}A_{M}A_{F}C_{M}A_{F} | | GTT | |
| | | | | _{M}^{S}G_{F}-C7OH- | | | | | G_{M}A_{F}G_{M}^{S}T_{D}^{S}T_{D}- | | | |
| | | | | [_{D}Tx-01-32] | | | | | OH-3′ | | | |
| DT- | 420 | 438 | DTS- | 5′-OH- | 650 | GUCGAU | 1075 | DTS- | 5′-PO4- | 158 | AAUGCU | 514 |
| 000621 | | | 000951 | G_{F}^{S}U_{M}^{S}C_{F}G_{M}A_{F}U | | CAUCUUC | | 000581 | A_{M}^{S}A_{F}^{S}U_{M}G_{F}C_{M} | | GAAGAU | |
| | | | | _{M}C_{F}A_{M}U_{F}C_{M}U_{F}U | | AGCAUU | | | U_{F}G_{M}A_{F}A_{M}G_{F}A | | GAUCGA | |
| | | | | _{M}C_{F}A_{M}G_{F}C_{M}A_{F}^{S}U | | | | | _{M}U_{F}G_{M}A_{F}U_{M}C_{F} | | CTT | |
| | | | | _{M}^{S}U_{F}-C7OH- | | | | | G_{M}A_{F}C_{M}^{S}T_{D}^{S}T_{D}- | | | |
| | | | | [_{D}Tx-01-08] | | | | | OH-3′ | | | |
| DT- | 499 | 517 | DTS- | 5′-OH- | 651 | AUCACU | 996 | DTS- | 5′-PO4- | 160 | UUUGGA | 639 |
| 000622 | | | 000952 | A_{F}^{S}U_{M}^{S}C_{F}A_{M}C_{F}U | | GGAAUC | | 000585 | U_{M}^{S}U_{F}^{S}U_{M}G_{F}G_{M} | | AGAUUC | |
| | | | | _{M}G_{F}G_{M}A_{F}A_{M}U_{F}C | | UUCCAA | | | A_{F}A_{M}G_{F}A_{M}U_{F}U | | CAGUGA | |
| | | | | _{M}U_{F}U_{M}C_{F}C_{M}A_{F}^{S}A | | A | | | _{M}C_{F}C_{M}A_{F}G_{M}U_{F} | | UTT | |
| | | | | _{M}^{S}A_{F}-C7OH- | | | | | G_{M}A_{F}U_{M}^{S}T_{D}^{S}T_{D}- | | | |
| | | | | [_{D}Tx-01-08] | | | | | OH-3′ | | | |
| DT- | 215 | 233 | DTS- | 5′-OH- | 652 | UCCUGU | 1090 | DTS- | 5′-PO4- | 176 | AUGAUA | 554 |
| 000623 | | | 000953 | U_{F}^{S}C_{M}^{S}C_{F}U_{M}G_{F}U | | UGCUGA | | 000617 | A_{M}^{S}U_{F}^{S}G_{M}A_{F}U_{M} | | CUCAGCA | |
| | | | | _{M}U_{F}G_{M}C_{F}U_{M}G_{F}A | | GUAUCA | | | A_{F}C_{M}U_{F}C_{M}A_{F}G | | ACAGGAT | |
| | | | | _{M}G_{F}U_{M}A_{F}U_{M}C_{F}^{S}A | | U | | | _{M}C_{F}A_{M}A_{F}C_{M}A_{F} | | T | |
| | | | | _{M}^{S}U_{F}-C7OH- | | | | | G_{M}G_{F}A_{M}^{S}T_{D}^{S}T_{D}- | | | |
| | | | | [_{D}Tx-01-08] | | | | | OH-3′ | | | |
| DT- | 370 | 388 | DTS- | 5′-OH- | 653 | UCAUCA | 1085 | DTS- | 5′-PO4- | 180 | AUUCGU | 569 |
| 000624 | | | 000954 | U_{F}^{S}C_{M}^{S}A_{F}U_{M}C_{F}A | | UCACCAA | | 000625 | A_{M}^{S}U_{F}^{S}U_{M}C_{F}G_{M} | | UUGGUG | |
| | | | | _{M}U_{F}C_{M}A_{F}C_{M}C_{F}A_{M} | | ACGAAU | | | U_{F}U_{M}U_{F}G_{M}G_{F}U | | AUGAUG | |
| | | | | A_{F}A_{M}C_{F}G_{M}A_{F}A_{M} | | | | | _{M}G_{F}A_{M}U_{F}G_{M}A_{F} | | ATT | |
| | | | | ^{S}U_{F}-C7OH- | | | | | U_{M}G_{F}A_{M}^{S}T_{D}^{S}T_{D}- | | | |
| | | | | [_{D}Tx-01-08] | | | | | OH-3′ | | | |
| DT- | 431 | 449 | DTS- | 5′-OH- | 654 | UCAGCA | 1084 | DTS- | 5′-PO4- | 182 | AACAGA | 498 |
| 000625 | | | 000955 | U_{F}^{S}C_{M}^{S}A_{F}G_{M}C_{F}A | | UUCUGU | | 000629 | A_{M}^{S}A_{F}^{S}C_{M}A_{F}G_{M} | | GACAGA | |
| | | | | _{M}U_{F}U_{M}C_{F}U_{M}G_{F}U | | CUCUGU | | | A_{F}G_{M}A_{F}C_{M}A_{F}G | | AUGCUG | |
| | | | | _{M}C_{F}U_{M}C_{F}U_{M}G_{F}^{S}U | | U | | | _{M}A_{F}A_{M}U_{F}G_{M}C_{F} | | ATT | |
| | | | | _{M}^{S}U_{F}-C7OH- | | | | | U_{M}G_{F}A_{M}^{S}T_{D}^{S}T_{D}- | | | |
| | | | | [_{D}Tx-01-08] | | | | | OH-3′ | | | |
| DT- | 442 | 460 | DTS- | 5′-OH- | 655 | UCUCUG | 1094 | DTS- | 5′-PO4- | 183 | AGAAGA | 529 |
| 000626 | | | 000956 | U_{F}^{S}C_{M}^{S}U_{F}C_{M}U_{F}G | | UUCCUG | | 000631 | A_{M}^{S}G_{F}^{S}A_{M}A_{F}G_{M} | | ACAGGA | |
| | | | | _{M}U_{F}U_{M}C_{F}C_{M}U_{F}G | | UUCUUC | | | A_{F}A_{M}C_{F}A_{M}G_{F}G | | ACAGAG | |
| | | | | _{M}U_{F}U_{M}C_{F}U_{M}U_{F}^{S}C | | U | | | _{M}A_{F}A_{M}C_{F}A_{M}G_{F} | | ATT | |
| | | | | _{M}^{S}U_{F}-C7OH- | | | | | A_{M}G_{F}A_{M}^{S}T_{D}^{S}T_{D}- | | | |
| | | | | [_{D}Tx-01-08] | | | | | OH-3′ | | | |
| DT- | 500 | 518 | DTS- | 5′-OH- | 656 | UCACUG | 1083 | DTS- | 5′-PO4- | 184 | AUUUGG | 575 |
| 000627 | | | 000957 | U_{F}^{S}C_{M}^{S}A_{F}C_{M}U_{F}G | | GAAUCU | | 000633 | A_{M}^{S}U_{F}^{S}U_{M}U_{F}G_{M} | | AAGAUU | |
| | | | | _{M}G_{F}A_{M}A_{F}U_{M}C_{F}U | | UCCAAA | | | G_{F}A_{M}A_{F}G_{M}A_{F}U | | CCAGUG | |
| | | | | _{M}U_{F}C_{M}C_{F}A_{M}A_{F}^{S}A | | U | | | _{M}U_{F}C_{M}C_{F}A_{M}G_{F} | | ATT | |
| | | | | _{M}^{S}U_{F}-C7OH- | | | | | U_{M}G_{F}A_{M}^{S}T_{D}^{S}T_{D}- | | | |
| | | | | [_{D}Tx-01-08] | | | | | OH-3′ | | | |
| DT- | 648 | 666 | DTS- | 5′-OH- | 657 | UCUCAGC | 1093 | DTS- | 5′-PO4- | 187 | AUAGAU | 543 |
| 000628 | | | 000958 | U_{F}^{S}C_{M}^{S}U_{F}C_{M}A_{F}G | | GGUGUC | | 000639 | A_{M}^{S}U_{F}^{S}A_{M}G_{F}A_{M} | | GACACCG | |
| | | | | _{M}C_{F}G_{M}G_{F}U_{M}G_{F}U | | AUCUAU | | | U_{F}G_{M}A_{F}C_{M}A_{F}C | | CUGAGAT | |
| | | | | _{M}C_{F}A_{M}U_{F}C_{M}U_{F}^{S}A | | | | | _{M}C_{F}G_{M}C_{F}U_{M}G_{F} | | T | |
| | | | | _{M}^{S}U_{F}-C7OH- | | | | | A_{M}G_{F}A_{M}^{S}T_{D}^{S}T_{D}- | | | |
| | | | | [_{D}Tx-01-08] | | | | | OH-3′ | | | |
| DT- | 215 | 233 | DTS- | 5′-OH- | 652 | UCCUGU | 1090 | DTS- | 5′-VP- | 188 | AUGAUA | 554 |
| 000811 | | | 000953 | U_{F}^{S}C_{M}^{S}C_{F}U_{M}G_{F}U | | UGCUGA | | 001216 | A_{M}^{S}U_{F}^{S}G_{M}A_{F}U_{M} | | CUCAGCA | |
| | | | | _{M}U_{F}G_{M}C_{F}U_{M}G_{F}A | | GUAUCA | | | A_{F}C_{M}U_{F}C_{M}A_{F}G | | ACAGGAT | |
| | | | | _{M}G_{F}U_{M}A_{F}U_{M}C_{E}^{S}A | | U | | | _{M}C_{F}A_{M}A_{F}C_{M}A_{F} | | T | |
| | | | | _{M}^{S}U_{F}-C7OH- | | | | | G_{M}G_{F}A_{M}^{S}T_{D}^{S}T_{D}- | | | |
| | | | | [_{D}Tx-01-08] | | | | | OH-3′ | | | |
| DT- | 213 | 233 | DTS- | 5′-OH- | 658 | CCUCCUG | 1018 | DTS- | 5′-VP- | 879 | AUGAUA | 1144 |
| 000812 | | | 001217 | C_{F}^{S}C_{M}^{S}U_{F}C_{M}C_{F}U | | UUGCUG | | 001218 | A_{M}^{S}U_{F}^{S}G_{M}A_{F}U_{M} | | CUCAGCA | |
| | | | | _{M}G_{F}U_{M}U_{F}G_{M}C_{F}U | | AGUAUC | | | A_{F}C_{M}U_{F}C_{M}A_{F}G | | ACAGGA | |
| | | | | _{M}G_{F}A_{M}G_{F}U_{M}A_{F}U | | AU | | | _{M}C_{F}A_{M}A_{F}C_{M}A_{F} | | GGAG | |
| | | | | _{M}C_{F}^{S}A_{M}^{S}U_{F}- | | | | | G_{M}G_{F}A_{M}G_{F}G^{S} | | | |
| | | | | C7OH-[_{D}Tx-01- | | | | | A_{M}^{S}G_{M}-OH-3′ | | | |
| | | | | 08] | | | | | | | | |
| DT- | 213 | 233 | DTS- | 5′-OH- | 658 | CCUCCUG | 1018 | DTS- | 5′-PO4- | 880 | AUGAUA | 1144 |
| 000945 | | | 001217 | C_{F}^{S}C_{M}^{S}U_{F}C_{M}C_{F}U | | UUGCUG | | 001454 | A_{M}^{S}U_{F}^{S}G_{M}A_{F}U_{M} | | CUCAGCA | |
| | | | | _{M}G_{F}U_{M}U_{F}G_{M}C_{F}U | | AGUAUC | | | A_{F}C_{M}U_{F}C_{M}A_{F}G | | ACAGGA | |
| | | | | _{M}G_{F}A_{M}G_{F}U_{M}A_{F}U | | AU | | | _{M}C_{F}A_{M}A_{F}C_{M}A_{F} | | GGAG | |
| | | | | _{M}C_{F}^{S}A_{M}^{S}U_{F}- | | | | | G_{M}G_{F}A_{M}G_{F}G_{M}^{S} | | | |
| | | | | C7OH-[_{D}Tx-01- | | | | | A_{M}^{S}G_{M}-OH-3′ | | | |
| | | | | 08] | | | | | | | | |
| DT- | 210 | 218 | DTS- | 5′-OH- | 659 | GCUCCUC | 1059 | DTS- | 5′-PO4- | 189 | ACUCAGC | 526 |
| 000959 | | | 001470 | G_{F}^{S}C_{M}^{S}U_{F}C_{M}C_{F}U | | CUGUUG | | 001264 | A_{M}^{S}C_{F}^{S}U_{M}C_{F}A_{M} | | AACAGG | |
| | | | | _{M}C_{F}C_{M}U_{F}G_{M}U_{F}U | | CUGAGU | | | G_{F}C_{M}A_{F}A_{M}C_{F}A | | AGGAGCT | |
| | | | | _{M}G_{F}C_{M}U_{F}G_{M}A_{F}^{S}G | | | | | _{M}G_{F}G_{M}A_{F}G_{M}G_{F} | | T | |
| | | | | _{M}^{S}U_{F}-C7OH- | | | | | A_{M}G_{F}C_{M}^{S}T_{D}^{S}T_{D}- | | | |
| | | | | [_{D}Tx-01-08] | | | | | OH-3′ | | | |
| DT- | 212 | 230 | DTS- | 5′-OH- | 660 | UCCUCCU | 1087 | DTS- | 5′-PO4- | 190 | AUACUC | 542 |
| 000960 | | | 001471 | U_{F}^{S}C_{M}^{S}C_{F}U_{M}C_{F}C | | GUUGCU | | 001266 | A_{M}^{S}U_{F}^{S}A_{M}C_{F}U_{M} | | AGCAAC | |
| | | | | _{M}U_{F}G_{M}U_{F}U_{M}G_{F}C | | GAGUAU | | | C_{F}A_{M}G_{F}C_{M}A_{F}A | | AGGAGG | |
| | | | | _{M}U_{F}G_{M}A_{F}G_{M}U_{F}^{S}A | | | | | _{M}C_{F}A_{M}G_{F}G_{M}A_{F} | | | |
| | | | | _{M}^{S}U_{F}-C7OH- | | | | | G_{M}G_{F}A_{M}^{S}T_{D}^{S}T_{D}- | | | |
| | | | | [_{D}Tx-01-08] | | | | | OH-3′ | | ATT | |
| DT- | 213 | 231 | DTS- | 5′-OH- | 661 | CCUCCUG | 1017 | DTS- | 5′-PO4- | 191 | GAUACU | 582 |
| 000961 | | | 001472 | C_{F}^{S}C_{M}^{S}U_{F}C_{M}C_{F}U | | UUGCUG | | 001268 | G_{M}^{S}A_{F}^{S}U_{M}A_{F}C_{M} | | CAGCAAC | |
| | | | | _{M}G_{F}U_{M}U_{F}G_{M}C_{F}U | | AGUAUC | | | U_{F}C_{M}A_{F}G_{M}C_{F}A | | AGGAGG | |
| | | | | _{M}G_{F}A_{M}G_{F}U_{M}A_{F}^{S}U | | | | | _{M}A_{F}C_{M}A_{F}G_{M}G_{F} | | TT | |
| | | | | _{M}^{S}U_{F}-C7OH- | | | | | A_{M}G_{F}G_{M}^{S}T_{D}^{S}T_{D}- | | | |
| | | | | [_{D}Tx-01-08] | | | | | OH-3′ | | | |
| DT- | 216 | 234 | DTS- | 5′-OH- | 662 | CCUGUU | 1023 | DTS- | 5′-PO4- | 193 | GAUGAU | 583 |
| 000962 | | | 001473 | C_{F}^{S}C_{M}^{S}U_{F}G_{M}U_{F}U | | GCUGAG | | 001272 | G_{M}^{S}A_{F}^{S}U_{M}G_{F}A_{M} | | ACUCAGC | |
| | | | | _{M}G_{F}C_{M}U_{F}G_{M}A_{F}G | | UAUCAU | | | U_{F}A_{M}C_{F}U_{M}C_{F}A | | AACAGGT | |
| | | | | _{M}U_{F}A_{M}U_{F}C_{M}A_{F}^{S}U | | C | | | _{M}G_{F}C_{M}A_{F}A_{M}C_{F} | | T | |
| | | | | _{M}^{S}U_{F}-C7OH- | | | | | A_{M}G_{F}G_{M}^{S}T_{D}^{S}T_{D}- | | | |
| | | | | [_{D}Tx-01-08] | | | | | OH-3′ | | | |
| DT- | 220 | 238 | DTS- | 5′-OH- | 663 | UUGCUG | 1106 | DTS- | 5′-PO4- | 197 | GGACGA | 585 |
| 000963 | | | 001474 | U_{F}^{S}U_{M}^{S}G_{F}C_{M}U_{F}G | | AGUAUC | | 001280 | G_{M}^{S}G_{F}^{S}A_{M}C_{F}G_{M} | | UGAUAC | |
| | | | | _{M}A_{F}G_{M}U_{F}A_{M}U_{F}C | | AUCGUCC | | | A_{F}U_{M}G_{F}A_{M}U_{F}A | | UCAGCA | |
| | | | | _{M}A_{F}U_{M}C_{F}G_{M}U_{F}^{S}C | | | | | _{M}C_{F}U_{M}C_{F}A_{M}G_{F} | | ATT | |
| | | | | _{M}^{S}U_{F}-C7OH- | | | | | C_{M}A_{F}A_{M}^{S}T_{D}^{S}T_{D}- | | | |
| | | | | [_{D}Tx-01-08] | | | | | OH-3′ | | | |
| DT- | 300 | 318 | DTS- | 5′-OH- | 664 | CAAUGG | 1004 | DTS- | 5′-PO4- | 209 | AUCAGU | 547 |
| 000964 | | | 001475 | C_{F}^{S}A_{M}^{S}A_{F}U_{M}G_{F}G | | ACACGCA | | 001304 | A_{M}^{S}U_{F}^{S}C_{M}A_{F}G_{M} | | UGCGUG | |
| | | | | _{M}A_{F}C_{M}A_{F}C_{M}G_{F}C_{M} | | ACUGAU | | | U_{F}U_{M}G_{F}C_{M}G_{F}U | | UCCAUU | |
| | | | | A_{F}A_{M}C_{F}U_{M}G_{F}^{S}A_{M} | | | | | _{M}G_{F}U_{M}C_{F}C_{M}A_{F} | | GTT | |
| | | | | ^{S}U_{F}-C7OH-[_{D}Tx- | | | | | U_{M}U_{F}G_{M}^{S}T_{D}^{S}T_{D}- | | | |
| | | | | 01-08] | | | | | OH-3′ | | | |
| DT- | 407 | 425 | DTS- | 5′-OH- | 665 | CCACCAU | 1012 | DTS- | 5′-PO4- | 217 | AUCGAC | 550 |
| 000965 | | | 001476 | C_{F}^{S}C_{M}^{S}A_{F}C_{M}C_{F}A | | GAUCCU | | 001320 | A_{M}^{S}U_{F}^{S}C_{M}G_{F}A_{M} | | AGGAUC | |
| | | | | _{M}U_{F}G_{M}A_{F}U_{M}C_{F}C | | GUCGAU | | | C_{F}A_{M}G_{F}G_{M}A_{F}U | | AUGGUG | |
| | | | | _{M}U_{F}G_{M}U_{F}C_{M}G_{F}^{S}A | | | | | _{M}C_{F}A_{M}U_{F}G_{M}G_{F} | | GTT | |
| | | | | _{M}^{S}U_{F}-C7OH- | | | | | U_{M}G_{F}G_{M}^{S}T_{D}^{S}T_{D}- | | | |
| | | | | [_{D}Tx-01-08] | | | | | OH-3′ | | | |
| DT- | 419 | 437 | DTS- | 5′-OH- | 666 | UGUCGA | 1100 | DTS- | 5′-PO4- | 219 | AUGCUG | 560 |
| 000966 | | | 001477 | U_{F}^{S}G_{M}^{S}U_{F}C_{M}G_{F}A | | UCAUCU | | 001324 | A_{M}^{S}U_{F}^{S}G_{M}C_{F}U_{M} | | AAGAUG | |
| | | | | _{M}U_{F}C_{M}A_{F}U_{M}C_{F}U | | UCAGCA | | | G_{F}A_{M}A_{F}G_{M}A_{F}U | | AUCGAC | |
| | | | | _{M}U_{F}C_{M}A_{F}G_{M}C_{F}^{S}A | | U | | | _{M}G_{F}A_{M}U_{F}C_{M}G_{F} | | ATT | |
| | | | | _{M}^{S}U_{F}-C7OH- | | | | | A_{M}C_{F}A_{M}^{S}T_{D}^{S}T_{D}- | | | |
| | | | | [_{D}Tx-01-08] | | | | | OH-3′ | | | |
| DT- | 449 | 467 | DTS- | 5′-OH- | 667 | UCCUGU | 1089 | DTS- | 5′-PO4- | 220 | AGUUGG | 538 |
| 000967 | | | 001478 | U_{F}^{S}C_{M}^{S}C_{F}U_{M}G_{F}U | | UCUUCU | | 001326 | A_{M}^{S}G_{F}^{S}U_{M}U_{F}G_{M} | | CAGAAG | |
| | | | | _{M}U_{F}C_{M}U_{F}U_{M}C_{F}U | | GCCAACU | | | G_{F}C_{M}A_{F}G_{M}A_{F}A | | AACAGG | |
| | | | | _{M}G_{F}C_{M}C_{F}A_{M}A_{F}^{S}C | | | | | _{M}G_{F}A_{M}A_{F}C_{M}A_{F} | | ATT | |
| | | | | _{M}^{S}U_{F}-C7OH- | | | | | G_{M}G_{F}A_{M}^{S}T_{D}^{S}T_{D}- | | | |
| | | | | [_{D}Tx-01-08] | | | | | OH-3′ | | | |
| DT- | 213 | 233 | DTS- | 5′-OH- | 668 | CCUCCUG | 1018 | DTS- | 5′-VP- | 879 | AUGAUA | 1144 |
| 001037 | | | 001615 | C_{F}^{S}C_{M}^{S}U_{F}C_{M}C_{F}U | | UUGCUG | | 001218 | A_{M}^{S}U_{F}^{S}G_{M}A_{F}U_{M} | | CUCAGCA | |
| | | | | _{M}G_{F}U_{M}U_{F}G_{M}C_{F}U | | AGUAUC | | | A_{F}C_{M}U_{F}C_{M}A_{F}G | | ACAGGA | |
| | | | | _{M}G_{F}A_{M}G_{F}U_{M}A_{F}U | | AU | | | _{M}C_{F}A_{M}A_{F}C_{M}A_{F} | | GGAG | |
| | | | | _{M}C_{F}^{S}A_{M}^{S}U_{F}- | | | | | G_{M}G_{F}A_{M}G_{F}G_{M}^{S} | | | |
| | | | | C7OH-[_{D}Tx-01- | | | | | A_{M}^{S}G_{M}-OH-3′ | | | |
| | | | | 32] | | | | | | | | |
| DT- | 494 | 512 | DTS- | 5′-OH- | 669 | UUUACA | 1109 | DTS- | 5′-VP- | 298 | AAGAUU | 506 |
| 001038 | | | 001616 | U_{F}^{S}U_{M}^{S}U_{F}A_{M}C_{F}A | | UCACUG | | 001618 | A_{M}^{S}A_{F}^{S}G_{M}A_{F}U_{M} | | CCAGUG | |
| | | | | _{M}U_{F}C_{M}A_{F}C_{M}U_{F}G | | GAAUCU | | | U_{F}C_{M}C_{F}A_{M}G_{F}U | | AUGUAA | |
| | | | | _{M}G_{F}A_{M}A_{F}U_{M}C_{F}^{S}U | | U | | | _{M}G_{F}A_{M}U_{F}G_{M}U_{F} | | ATT | |
| | | | | _{M}^{S}U_{F}-C7OH- | | | | | A_{M}A_{F}A_{M}^{S}T_{D}^{S}T_{D}- | | | |
| | | | | [_{D}Tx-01-08] | | | | | OH-3′ | | | |
| DT- | 494 | 512 | DTS- | 5′-OH- | 670 | UUUACA | 1109 | DTS- | 5′-VP- | 298 | AAGAUU | 506 |
| 001039 | | | 001617 | U_{F}^{S}U_{M}^{S}U_{F}A_{M}C_{F}A | | UCACUG | | 001618 | A_{M}^{S}A_{F}^{S}G_{M}A_{F}U_{M} | | CCAGUG | |
| | | | | _{M}U_{F}C_{M}A_{F}C_{M}U_{F}G | | GAAUCU | | | U_{F}C_{M}C_{F}A_{M}G_{F}U | | AUGUAA | |
| | | | | _{M}G_{F}A_{M}A_{F}U_{M}C_{F}^{S}U | | U | | | _{M}G_{F}A_{M}U_{F}G_{M}U_{F} | | ATT | |
| | | | | _{M}^{S}U_{F}-C7OH- | | | | | A_{M}A_{F}A_{M}^{S}T_{D}^{S}T_{D}- | | | |
| | | | | [_{D}Tx-01-32] | | | | | OH-3′ | | | |
| DT- | 464 | 482 | DTS- | 5′-OH- | 671 | AACUCU | 980 | DTS- | 5′-VP- | 299 | UUGGUG | 628 |
| 001044 | | | 001625 | A_{F}^{S}A_{M}^{S}C_{F}U_{M}C_{F}U | | UCACCCU | | 001643 | U_{M}^{S}U_{F}^{S}G_{M}G_{F}U_{M} | | AGGGUG | |
| | | | | _{M}U_{F}C_{M}A_{F}C_{M}C_{F}C_{M} | | CACCAA | | | G_{F}A_{M}G_{F}G_{M}G_{F}U | | AAGAGU | |
| | | | | U_{F}C_{M}A_{F}C_{M}C_{F}^{S}A_{M} | | | | | _{M}G_{F}A_{M}A_{F}G_{M}A_{F} | | UTT | |
| | | | | ^{S}A_{F}-C7OH-[_{D}Tx- | | | | | G_{M}U_{F}U_{M}^{S}T_{D}^{S}T_{D}- | | | |
| | | | | 01-32] | | | | | OH-3′ | | | |
| DT- | 486 | 504 | DTS- | 5′-OH- | 672 | GGGCAG | 1070 | DTS- | 5′-VP- | 300 | AGUGAU | 536 |
| 001045 | | | 001626 | G_{F}^{S}G_{M}^{S}G_{F}C_{M}A_{F}G | | GUUUUA | | 001644 | A_{M}^{S}G_{F}^{S}U_{M}G_{F}A_{M} | | GUAAAA | |
| | | | | _{M}G_{F}U_{M}U_{F}U_{M}U_{F}A | | CAUCACU | | | U_{F}G_{M}U_{F}A_{M}A_{F}A | | CCUGCCC | |
| | | | | _{M}C_{F}A_{M}U_{F}C_{M}A_{F}^{S}C | | | | | _{M}A_{F}C_{M}C_{F}U_{M}G_{F} | | TT | |
| | | | | _{M}^{S}U_{F}-C7OH- | | | | | C_{M}C_{F}C_{M}^{S}T_{D}^{S}T_{D}- | | | |
| | | | | [_{D}Tx-01-32] | | | | | OH-3′ | | | |
| DT- | 491 | 509 | DTS- | 5′-OH- | 673 | GGUUUU | 1072 | DTS- | 5′-VP- | 301 | AUUCCA | 567 |
| 001046 | | | 001627 | G_{F}^{S}G_{M}^{S}U_{F}U_{M}U_{F}U | | ACAUCAC | | 001645 | A_{M}^{S}U_{F}^{S}U_{M}C_{F}C_{M} | | GUGAUG | |
| | | | | _{M}A_{F}C_{M}A_{F}U_{M}C_{F}A | | UGGAAU | | | A_{F}G_{M}U_{F}G_{M}A_{F}U | | UAAAAC | |
| | | | | _{M}C_{F}U_{M}G_{F}G_{M}A_{F}^{S}A | | | | | _{M}G_{F}U_{M}A_{F}A_{M}A_{F} | | CTT | |
| | | | | _{M}^{S}U_{F}-C7OH- | | | | | A_{M}C_{F}C_{M}^{S}T_{D}^{S}T_{D}- | | | |
| | | | | [_{D}Tx-01-32] | | | | | OH-3′ | | | |
| DT- | 501 | 519 | DTS- | 5′-OH- | 674 | CACUGG | 1006 | DTS- | 5′-VP- | 302 | AAUUUG | 515 |
| 001047 | | | 001628 | C_{F}^{S}A_{M}^{S}C_{F}U_{M}G_{F}G | | AAUCUU | | 001646 | A_{M}^{S}A_{F}^{S}U_{M}U_{F}U_{M} | | GAAGAU | |
| | | | | _{M}A_{F}A_{M}U_{F}C_{M}U_{F}U | | CCAAAU | | | G_{F}G_{M}A_{F}A_{M}G_{F}A | | UCCAGU | |
| | | | | _{M}C_{F}C_{M}A_{F}A_{M}A_{F}^{S}U | | U | | | _{M}U_{F}U_{M}C_{F}C_{M}A_{F} | | GTT | |
| | | | | _{M}^{S}U_{F}-C7OH- | | | | | G_{M}U_{F}G_{M}^{S}T_{D}^{S}T_{D}- | | | |
| | | | | [_{D}Tx-01-32] | | | | | OH-3′ | | | |
| DT- | 507 | 525 | DTS- | 5′-OH- | 675 | AAUCUU | 983 | DTS- | 5′-VP- | 303 | AGCAAG | 533 |
| 001048 | | | 001629 | A_{F}^{S}A_{M}^{S}U_{F}C_{M}U_{F}U | | CCAAAU | | 001647 | A_{M}^{S}G_{F}^{S}C_{M}A_{F}A_{M} | | AAUUUG | |
| | | | | _{M}C_{F}C_{M}A_{F}A_{M}A_{F}U | | UCUUGC | | | G_{F}A_{M}A_{F}U_{M}U_{F}U | | GAAGAU | |
| | | | | _{M}U_{F}C_{M}U_{F}U_{M}G_{F}^{S}C | | U | | | _{M}G_{F}G_{M}A_{F}A_{M}G_{F} | | UTT | |
| | | | | _{M}^{S}U_{F}-C7OH- | | | | | A_{M}U_{F}U_{M}^{S}T_{D}^{S}T_{D}- | | | |
| | | | | [_{D}Tx-01-32] | | | | | OH-3′ | | | |
| DT- | 514 | 532 | DTS- | 5′-OH- | 676 | CAAAUU | 1002 | DTS- | 5′-VP- | 304 | ACAGACC | 516 |
| 001049 | | | 001630 | C_{F}^{S}A_{M}^{S}A_{F}A_{M}U_{F}U | | CUUGCU | | 001648 | A_{M}^{S}C_{F}^{S}A_{M}G_{F}A_{M} | | AGCAAG | |
| | | | | _{M}C_{F}U_{M}U_{F}G_{M}C_{F}U | | GGUCUG | | | C_{F}C_{M}A_{F}G_{M}C_{F}A | | AAUUUG | |
| | | | | _{M}G_{F}G_{M}U_{F}C_{M}U_{F}^{S}G | | U | | | _{M}A_{F}G_{M}A_{F}A_{M}U_{F} | | TT | |
| | | | | _{M}^{S}U_{F}-C7OH- | | | | | U_{M}U_{F}G_{M}^{S}T_{D}^{S}T_{D}- | | | |
| | | | | [_{D}Tx-01-32] | | | | | OH-3′ | | | |
| DT- | 523 | 541 | DTS- | 5′-OH- | 677 | GCUGGU | 1061 | DTS- | 5′-VP- | 305 | UCAUCAC | 600 |
| 001050 | | | 001631 | G_{F}^{S}C_{M}^{S}U_{F}G_{M}G_{F}U | | CUGUGC | | 001649 | U_{M}^{S}C_{F}^{S}A_{M}U_{F}C_{M} | | GCACAG | |
| | | | | _{M}C_{F}U_{M}G_{F}U_{M}G_{F}C | | GUGAUG | | | A_{F}C_{M}G_{F}C_{M}A_{F}C | | ACCAGCT | |
| | | | | _{M}G_{F}U_{M}G_{F}A_{M}U_{F}^{S}G | | A | | | _{M}A_{F}G_{M}A_{F}C_{M}C_{F} | | T | |
| | | | | _{M}^{S}A_{F}-C7OH- | | | | | A_{M}G_{F}C_{M}^{S}T_{D}^{S}T_{D}- | | | |
| | | | | [_{D}Tx-01-32] | | | | | OH-3′ | | | |
| DT- | 536 | 554 | DTS- | 5′-OH- | 678 | UGAUGA | 1096 | DTS- | 5′-VP- | 306 | AUGGCC | 561 |
| 001051 | | | 001632 | U_{F}^{S}G_{M}^{S}A_{F}U_{M}G_{F}A | | GUGCUG | | 001650 | A_{M}^{S}U_{F}^{S}G_{M}G_{F}C_{M} | | GCAGCAC | |
| | | | | _{M}G_{F}U_{M}G_{F}C_{M}U_{F}G | | CGGCCAU | | | C_{F}G_{M}C_{F}A_{M}G_{F}C | | UCAUCAT | |
| | | | | _{M}C_{F}G_{M}G_{F}C_{M}C_{F}^{S}A | | | | | _{M}A_{F}C_{M}U_{F}C_{M}A_{F} | | T | |
| | | | | _{M}^{S}U_{F}-C7OH- | | | | | U_{M}C_{F}A_{M}^{S}T_{D}^{S}T_{D}- | | | |
| | | | | [_{D}Tx-01-32] | | | | | OH-3′ | | | |
| DT- | 564 | 582 | DTS- | 5′-OH- | 679 | GAGGCA | 1053 | DTS- | 5′-VP- | 307 | AUGCCAC | 559 |
| 001052 | | | 001633 | G_{F}^{S}A_{M}^{S}G_{F}G_{M}C_{F}A | | CCCGGAG | | 001651 | A_{M}^{S}U_{F}^{S}G_{M}C_{F}C_{M} | | UCCGGG | |
| | | | | _{M}C_{F}C_{M}C_{F}G_{M}G_{F}A_{M} | | UGGCAU | | | A_{F}C_{M}U_{F}C_{M}C_{F}G | | UGCCUCT | |
| | | | | G_{F}U_{M}G_{F}G_{M}C_{F}^{S}A_{M} | | | | | _{M}G_{F}G_{M}U_{F}G_{M}C_{F} | | T | |
| | | | | ^{S}U_{F}-C7OH-[_{D}Tx- | | | | | C_{M}U_{F}C_{M}^{S}T_{D}^{S}T_{D}- | | | |
| | | | | 01-32] | | | | | OH-3′ | | | |
| DT- | 569 | 587 | DTS- | 5′-OH- | 680 | ACCCGGA | 987 | DTS- | 5′-VP- | 308 | UUGAGA | |
| 001053 | | | 001634 | A_{F}^{S}C_{M}^{S}C_{F}C_{M}G_{F}G | | GUGGCA | | 001652 | U_{M}^{S}U_{F}^{S}G_{M}A_{F}G_{M} | | UGCCACU | |
| | | | | _{M}A_{F}G_{M}U_{F}G_{M}G_{F}C | | UCUCAA | | | A_{F}U_{M}G_{F}C_{M}C_{F}A | | CCGGGUT | |
| | | | | _{M}A_{F}U_{M}C_{F}U_{M}C_{E}^{S}A | | | | | _{M}C_{F}U_{M}C_{F}C_{M}G_{F} | | T | |
| | | | | _{M}^{S}A_{F}-C7OH- | | | | | G_{M}G_{F}U_{M}^{S}T_{D}^{S}T_{D}- | | | |
| | | | | [_{D}Tx-01-32] | | | | | OH-3′ | | | 622 |
| DT- | 584 | 602 | DTS- | 5′-OH- | 681 | UCAACUC | 1082 | DTS- | 5′-VP- | 309 | UAGGAG | 595 |
| 001054 | | | 001635 | U_{F}^{S}C_{M}^{S}A_{F}A_{M}C_{F}U | | GGAUUA | | 001653 | U_{M}^{S}A_{F}^{S}G_{M}G_{F}A_{M} | | UAAUCC | |
| | | | | _{M}C_{F}G_{M}G_{F}A_{M}U_{F}U | | CUCCUA | | | G_{F}U_{M}A_{F}A_{M}U_{F}C | | GAGUUG | |
| | | | | _{M}A_{F}C_{M}U_{F}C_{M}C_{F}^{S}U | | | | | _{M}C_{F}G_{M}A_{F}G_{M}U_{F} | | ATT | |
| | | | | _{M}^{S}A_{F}-C7OH- | | | | | U_{M}G_{F}A_{M}^{S}T_{D}^{S}T_{D}- | | | |
| | | | | [_{D}Tx-01-32] | | | | | OH-3′ | | | |
| DT- | 590 | 608 | DTS- | 5′-OH- | 682 | CGGAUU | 1025 | DTS- | 5′-VP- | 310 | AAACCG | 493 |
| 001055 | | | 001636 | C_{F}^{S}G_{M}^{S}G_{F}A_{M}U_{F}U | | ACUCCUA | | 001654 | A_{M}^{S}A_{F}^{S}A_{M}C_{F}C_{M} | | UAGGAG | |
| | | | | _{M}A_{F}C_{M}U_{F}C_{M}C_{F}U_{M} | | CGGUUU | | | G_{F}U_{M}A_{F}G_{M}G_{F}A | | UAAUCC | |
| | | | | A_{F}C_{M}G_{F}G_{M}U_{F}^{S}U_{M} | | | | | _{M}G_{F}U_{M}A_{F}A_{M}U_{F} | | GTT | |
| | | | | ^{S}U_{F}-C7OH-[_{D}Tx- | | | | | C_{M}C_{F}G_{M}^{S}T_{D}^{S}T_{D}- | | | |
| | | | | 01-32] | | | | | OH-3′ | | | |
| DT- | 599 | 617 | DTS- | 5′-OH- | 683 | CCUACGG | 1016 | DTS- | 5′-VP- | 311 | AUGUAG | 563 |
| 001056 | | | 001637 | C_{F}^{S}C_{M}^{S}U_{F}A_{M}C_{F}G | | UUUCGCC | | 001655 | A_{M}^{S}U_{F}^{S}G_{M}U_{F}A_{M} | | GCGAAA | |
| | | | | _{M}G_{F}U_{M}U_{F}U_{M}C_{F}G | | UACAU | | | G_{F}G_{M}C_{F}G_{M}A_{F}A | | CCGUAG | |
| | | | | _{M}C_{F}C_{M}U_{F}A_{M}C_{F}^{S}A | | | | | _{M}A_{F}C_{M}C_{F}G_{M}U_{F} | | GTT | |
| | | | | _{M}^{S}U_{F}-C7OH- | | | | | A_{M}G_{F}G_{M}^{S}T_{D}^{S}T_{D}- | | | |
| | | | | [_{D}Tx-01-32] | | | | | OH-3′ | | | |
| DT- | 602 | 620 | DTS- | 5′-OH- | 684 | ACGGUU | 990 | DTS- | 5′-VP- | 312 | AGGAUG | 535 |
| 001057 | | | 001638 | A_{F}^{S}C_{M}^{S}G_{F}G_{M}U_{F}U | | UCGCCUA | | 001656 | A_{M}^{S}G_{F}^{S}G_{M}A_{F}U_{M} | | UAGGCG | |
| | | | | _{M}U_{F}C_{M}G_{F}C_{M}C_{F}U_{M} | | CAUCCU | | | G_{F}U_{M}A_{F}G_{M}G_{F}C | | AAACCG | |
| | | | | A_{F}C_{M}A_{F}U_{M}C_{F}^{S}C_{M} | | | | | _{M}G_{F}A_{M}A_{F}A_{M}C_{F} | | UTT | |
| | | | | ^{S}U_{F}-C7OH- | | | | | C_{M}G_{F}U_{M}^{S}T_{D}^{S}T_{D}- | | | |
| | | | | [_{D}Tx-01-32] | | | | | OH-3′ | | | |
| DT- | 644 | 662 | DTS- | 5′-OH- | 685 | CCCUUCU | 1014 | DTS- | 5′-VP- | 313 | AUGACA | 553 |
| 001058 | | | 001639 | C_{F}^{S}C_{M}^{S}C_{F}U_{M}U_{F}C | | CAGCGG | | 001657 | A_{M}^{S}U_{F}^{S}G_{M}A_{F}C_{M} | | CCGCUGA | |
| | | | | _{M}U_{F}C_{M}A_{F}G_{M}C_{F}G | | UGUCAU | | | A_{F}C_{M}C_{F}G_{M}C_{F}U | | GAAGGG | |
| | | | | _{M}G_{F}U_{M}G_{F}U_{M}C_{F}^{S}A | | | | | _{M}G_{F}A_{M}G_{F}A_{M}A_{F} | | TT | |
| | | | | _{M}^{S}U_{F}-C7OH- | | | | | G_{M}G_{F}G_{M}^{S}T_{D}^{S}T_{D}- | | | |
| | | | | [_{D}Tx-01-32] | | | | | OH-3′ | | | |
| DT- | 653 | 671 | DTS- | 5′-OH- | 686 | GCGGUG | 1058 | DTS- | 5′-VP- | 314 | AUCACA | 545 |
| 001059 | | | 001640 | G_{F}^{S}C_{M}^{S}G_{F}G_{M}U_{F}G | | UCAUCU | | 001658 | A_{M}^{S}U_{F}^{S}C_{M}A_{F}C_{M} | | UAGAUG | |
| | | | | _{M}U_{F}C_{M}A_{F}U_{M}C_{F}U | | AUGUGA | | | A_{F}U_{M}A_{F}G_{M}A_{F}U | | ACACCGC | |
| | | | | _{M}A_{F}U_{M}G_{F}U_{M}G_{F}^{S}A | | U | | | _{M}G_{F}A_{M}C_{F}A_{M}C_{F} | | TT | |
| | | | | _{M}^{S}U_{F}-C7OH- | | | | | C_{M}G_{F}C_{M}^{S}T_{D}^{S}T_{D}- | | | |
| | | | | [_{D}Tx-01-32] | | | | | OH-3′ | | | |
| DT- | 656 | 674 | DTS- | 5′-OH- | 687 | GUGUCA | 1076 | DTS- | 5′-VP- | 315 | AAGAUC | 504 |
| 001060 | | | 001641 | G_{F}^{S}U_{M}^{S}G_{F}U_{M}C_{F}A | | UCUAUG | | 001659 | A_{M}^{S}A_{F}^{S}G_{M}A_{F}U_{M} | | ACAUAG | |
| | | | | _{M}U_{F}C_{M}U_{F}A_{M}U_{F}G | | UGAUCU | | | C_{F}A_{M}C_{F}A_{M}U_{F}A | | AUGACA | |
| | | | | _{M}U_{F}G_{M}A_{F}U_{M}C_{F}^{S}U | | U | | | _{M}G_{F}A_{M}U_{F}G_{M}A_{F} | | CTT | |
| | | | | _{M}^{S}U_{F}-C7OH- | | | | | C_{M}A_{F}C_{M}^{S}T_{D}^{S}T_{D}- | | | |
| | | | | [_{D}Tx-01-32] | | | | | OH-3′ | | | |
| DT- | 663 | 681 | DTS- | 5′-OH- | 688 | CUAUGU | 1032 | DTS- | 5′-VP- | 316 | UUUCCGC | 631 |
| 001061 | | | 001642 | C_{F}^{S}U_{M}^{S}A_{F}U_{M}G_{F}U | | GAUCUU | | 001660 | U_{M}^{S}U_{F}^{S}U_{M}C_{F}C_{M} | | AAGAUC | |
| | | | | _{M}G_{F}A_{M}U_{F}C_{M}U_{F}U | | GCGGAA | | | G_{F}C_{M}A_{F}A_{M}G_{F}A | | ACAUAGT | |
| | | | | _{M}G_{F}C_{M}G_{F}G_{M}A_{F}^{S}A | | A | | | _{M}U_{F}C_{M}A_{F}C_{M}A_{F} | | T | |
| | | | | _{M}^{S}A_{F}-C7OH- | | | | | U_{M}A_{F}G_{M}^{S}T_{D}^{S}T_{D}- | | | |
| | | | | [_{D}Tx-01-32] | | | | | OH-3′ | | | |
| DT- | 737 | 755 | DTS- | 5′-OH- | 689 | GAGGAA | 1052 | DTS- | 5′-PO4- | 249 | UUUCUG | 635 |
| 001109 | | | 001743 | G_{F}^{S}A_{M}^{S}G_{F}G_{M}A_{F}A | | GGGAAA | | 001384 | U_{M}^{S}U_{F}^{S}U_{M}C_{F}U_{M} | | UUUUCCC | |
| | | | | _{M}G_{F}G_{M}G_{F}A_{M}A_{F}A | | ACAGAA | | | G_{F}U_{M}U_{F}U_{M}U_{F}C | | UUCCUCT | |
| | | | | _{M}A_{F}C_{M}A_{F}G_{M}A_{F}^{S}A | | A | | | _{M}C_{F}C_{M}U_{F}U_{M}C_{F}C | | T | |
| | | | | _{M}^{S}A_{F}-C7OH- | | | | | _{M}U_{F}C_{M}^{S}T_{D}^{S}T_{D}- | | | |
| | | | | [_{D}Tx-01-32] | | | | | OH-3 | | | |
| DT- | 779 | 797 | DTS- | 5′-OH- | 690 | CCCAAAA | 1013 | DTS- | 5′-PO4- | 250 | UUGAGU | 623 |
| 001110 | | | 001744 | C_{F}^{S}C_{M}^{S}C_{F}A_{M}A_{F}A | | UCCCAAA | | 001386 | U_{M}^{S}U_{F}^{S}G_{M}A_{F}G_{M} | | UUGGGA | |
| | | | | _{M}A_{F}U_{M}C_{F}C_{M}C_{F}A_{M} | | CUCAA | | | U_{F}U_{M}U_{F}G_{M}G_{F}G | | UUUUGG | |
| | | | | A_{F}A_{M}C_{F}U_{M}C_{F}^{S}A_{M} | | | | | _{M}A_{F}U_{M}U_{F}U_{M}U_{F} | | GTT | |
| | | | | ^{S}A_{F}-C7OH-[_{D}Tx- | | | | | G_{M}G_{F}G_{M}^{S}T_{D}^{S}T_{D}- | | | |
| | | | | 01-32] | | | | | OH-3′ | | | |
| DT- | 785 | 803 | DTS- | 5′-OH- | 691 | AUCCCAA | 998 | DTS- | 5′-PO4- | 251 | UUUGGU | |
| 001111 | | | 001745 | A_{F}^{S}U_{M}^{S}C_{F}C_{M}C_{F}A | | ACUCAA | | 001388 | U_{M}^{S}U_{F}^{S}U_{M}G_{F}G_{M} | | UUGAGU | |
| | | | | _{M}A_{F}A_{M}C_{F}U_{M}C_{F}A | | ACCAAA | | | U_{F}U_{M}U_{F}G_{M}A_{F}G | | UUGGGA | |
| | | | | _{M}A_{F}A_{M}C_{F}C_{M}A_{F}^{S}A | | | | | _{M}U_{F}U_{M}U_{F}G_{M}G_{F} | | UTT | |
| | | | | ^{S}A_{F}-C7OH- | | | | | G_{M}A_{F}U_{M}^{S}T_{D}^{S}T_{D}- | | | |
| | | | | [_{D}Tx-01-32] | | | | | OH-3′ | | | 641 |
| DT- | 833 | 851 | DTS- | 5′-OH- | 692 | GCUGUU | 1062 | DTS- | 5′-PO4- | 252 | UACAUC | 591 |
| 001112 | | | 001746 | G_{F}^{S}C_{M}^{S}U_{F}G_{M}U_{F}U | | GAUUGA | | 001390 | U_{M}^{S}A_{F}^{S}C_{M}A_{F}U_{M} | | UUCAAU | |
| | | | | _{M}G_{F}A_{M}U_{F}U_{M}G_{F}A | | AGAUGU | | | C_{F}U_{M}U_{F}C_{M}A_{F}A | | CAACAGC | |
| | | | | _{M}A_{F}G_{M}A_{F}U_{M}G_{F}^{S}U | | A | | | _{M}U_{F}C_{M}A_{F}A_{M}C_{F} | | TT | |
| | | | | _{M}^{S}A_{F}-C7OH- | | | | | A_{M}G_{F}C_{M}^{S}T_{D}^{S}T_{D}- | | | |
| | | | | [_{D}Tx-01-32] | | | | | OH-3′ | | | |
| DT- | 834 | 852 | DTS- | 5′-OH- | 693 | CUGUUG | 1043 | DTS- | 5′-PO4- | 253 | AUACAU | 541 |
| 001113 | | | 001747 | C_{F}^{S}U_{M}^{S}G_{F}U_{M}U_{F}G | | AUUGAA | | 001392 | A_{M}^{S}U_{F}^{S}A_{M}C_{F}A_{M} | | CUUCAA | |
| | | | | _{M}A_{F}U_{M}U_{F}G_{M}A_{F}A | | GAUGUA | | | U_{F}C_{M}U_{F}U_{M}C_{F}A | | UCAACA | |
| | | | | _{M}G_{F}A_{M}U_{F}G_{M}U_{F}^{S}A | | U | | | _{M}A_{F}U_{M}C_{F}A_{M}A_{F} | | GTT | |
| | | | | _{M}^{S}U_{F}-C7OH- | | | | | C_{M}A_{F}G_{M}^{S}T_{D}^{S}T_{D}- | | | |
| | | | | [_{D}Tx-01-32] | | | | | OH-3′ | | | |
| DT- | 865 | 883 | DTS- | 5′-OH- | 694 | GUUUAU | 1080 | DTS- | 5′-PO4- | 255 | AUAAAU | 539 |
| 001114 | | | 001748 | G_{F}^{S}U_{M}^{S}U_{F}U_{M}A_{F}U | | AAAACC | | 001396 | A_{M}^{S}U_{F}^{S}A_{M}A_{F}A_{M} | | AGGUUU | |
| | | | | _{M}A_{F}A_{M}A_{F}A_{M}C_{F}C | | UAUUUA | | | U_{F}A_{M}G_{F}G_{M}U_{F}U | | UAUAAA | |
| | | | | _{M}U_{F}A_{M}U_{F}U_{M}U_{F}^{S}A | | U | | | _{M}U_{F}U_{M}A_{F}U_{M}A_{F} | | CTT | |
| | | | | _{M}^{S}U_{F}-C7OH- | | | | | A_{M}A_{F}C_{M}^{S}T_{D}^{S}T_{D}- | | | |
| | | | | [_{D}Tx-01-32] | | | | | OH-3′ | | | |
| DT- | 904 | 922 | DTS- | 5′-OH- | 695 | ACAUAG | 985 | DTS- | 5′-PO4- | 256 | AAAGCA | 494 |
| 001115 | | | 001749 | A_{F}^{S}C_{M}^{S}A_{F}U_{M}A_{F}G | | UAUUGU | | 001398 | A_{M}^{S}A_{F}^{S}A_{M}G_{F}C_{M} | | AACAAU | |
| | | | | _{M}U_{F}A_{M}U_{F}U_{M}G_{F}U | | UUGCUU | | | A_{F}A_{M}A_{F}C_{M}A_{F}A | | ACUAUG | |
| | | | | _{M}U_{F}U_{M}G_{F}C_{M}U_{F}^{S}U | | U | | | _{M}U_{F}A_{M}C_{F}U_{M}A_{F} | | UTT | |
| | | | | _{M}^{S}U_{F}-C7OH- | | | | | U_{M}G_{F}U_{M}^{S}T_{D}^{S}T_{D}- | | | |
| | | | | [_{D}Tx-01-32] | | | | | OH-3′ | | | |
| DT- | 929 | 947 | DTS- | 5′-OH- | 696 | UGACCA | 1095 | DTS- | 5′-PO4- | 257 | AACACG | 497 |
| 001116 | | | 001750 | U_{F}^{S}G_{M}^{S}A_{F}C_{M}C_{F}A | | UCAGCCU | | 001400 | A_{M}^{S}A_{F}^{S}C_{M}A_{F}C_{M} | | AGGCUG | |
| | | | | _{M}U_{F}C_{M}A_{F}G_{M}C_{F}C_{M} | | CGUGUU | | | G_{F}A_{M}G_{F}G_{M}C_{F}U | | AUGGUC | |
| | | | | U_{F}C_{M}G_{F}U_{M}G_{F}^{S}U_{M} | | | | | _{M}G_{F}A_{M}U_{F}G_{M}G_{F} | | ATT | |
| | | | | ^{S}U_{F}-C7OH-[_{D}Tx- | | | | | U_{M}C_{F}A_{M}^{S}T_{D}^{S}T_{D}- | | | |
| | | | | 01-32] | | | | | OH-3′ | | | |
| DT- | 950 | 968 | DTS- | 5′-OH- | 697 | GCCUUA | 1057 | DTS- | 5′-PO4- | 258 | UUAGCU | 609 |
| 001117 | | | 001751 | G_{F}^{S}C_{M}^{S}C_{F}U_{M}U_{F}A | | AAGAAG | | 001402 | U_{M}^{S}U_{F}^{S}A_{M}G_{F}C_{M} | | ACUUCU | |
| | | | | _{M}A_{F}A_{M}G_{F}A_{M}A_{F}G | | UAGCUA | | | U_{F}A_{M}C_{F}U_{M}U_{F}C | | UUAAGG | |
| | | | | _{M}U_{F}A_{M}G_{F}C_{M}U_{F}^{S}A | | A | | | _{M}U_{F}U_{M}U_{F}A_{M}A_{F} | | CTT | |
| | | | | _{M}^{S}A_{F}-C7OH- | | | | | G_{M}G_{F}C_{M}^{S}T_{D}^{S}T_{D}- | | | |
| | | | | [_{D}Tx-01-32] | | | | | OH-3′ | | | |
| DT- | 958 | 976 | DTS- | 5′-OH- | 698 | GAAGUA | 1050 | DTS- | 5′-PO4- | 259 | AAAGUU | 495 |
| 001118 | | | 001752 | G_{F}^{S}A_{M}^{S}A_{F}G_{M}U_{F}A | | GCUAAG | | 001404 | A_{M}^{S}A_{F}^{S}A_{M}G_{F}U_{M} | | CCUUAGC | |
| | | | | _{M}G_{F}C_{M}U_{F}A_{M}A_{F}G | | GAACUU | | | U_{F}C_{M}C_{F}U_{M}U_{F}A | | UACUUCT | |
| | | | | _{M}G_{F}A_{M}A_{F}C_{M}U_{F}^{S}U | | U | | | _{M}G_{F}C_{M}U_{F}A_{M}C_{F} | | T | |
| | | | | _{M}^{S}U_{F}-C7OH- | | | | | U_{M}U_{F}C_{M}^{S}T_{D}^{S}T_{D}- | | | |
| | | | | [_{D}Tx-01-32] | | | | | OH-3′ | | | |
| DT- | 967 | 985 | DTS- | 5′-OH- | 699 | AAGGAA | 982 | DTS- | 5′-PO4- | 260 | UUAGGA | 610 |
| 001119 | | | 001753 | A_{F}^{S}A_{M}^{S}G_{F}G_{M}A_{F}A | | CUUUAC | | 001406 | U_{M}^{S}U_{F}^{S}A_{M}G_{F}G_{M} | | UGUAAA | |
| | | | | _{M}C_{F}U_{M}U_{F}U_{M}A_{F}C | | AUCCUA | | | A_{F}U_{M}G_{F}U_{M}A_{F}A | | GUUCCU | |
| | | | | _{M}A_{F}U_{M}C_{F}C_{M}U_{F}^{S}A | | A | | | _{M}A_{F}G_{M}U_{F}U_{M}C_{F} | | UTT | |
| | | | | _{M}^{S}A_{F}-C7OH- | | | | | C_{M}U_{F}U_{M}^{S}T_{D}^{S}T_{D}- | | | |
| | | | | [_{D}Tx-01-32] | | | | | OH-3′ | | | |
| DT- | 1786 | 1804 | DTS- | 5′-OH- | 700 | UGUGUG | 1101 | DTS- | 5′-PO4- | 272 | AUGCAU | 558 |
| 001120 | | | 001754 | U_{F}^{S}G_{M}^{S}U_{F}G_{M}U_{F}G | | GACUAA | | 001430 | A_{M}^{S}U_{F}^{S}G_{M}C_{F}A_{M} | | CUUAGU | |
| | | | | _{M}G_{F}A_{M}C_{F}U_{M}A_{F}A | | GAUGCA | | | U_{F}C_{M}U_{F}U_{M}A_{F}G | | CCACACA | |
| | | | | _{M}G_{F}A_{M}U_{F}G_{M}C_{F}^{S}A | | U | | | _{M}U_{F}C_{M}C_{F}A_{M}C_{E} | | TT | |
| | | | | _{M}^{S}U_{F}-C7OH- | | | | | A_{M}C_{F}A_{M}^{S}T_{D}^{S}T_{D}- | | | |
| | | | | [_{D}Tx-01-32] | | | | | OH-3′ | | | |
| DT- | 737 | 755 | DTS- | 5′-OH- | 689 | GAGGAA | 1052 | DTS- | 5′-VP- | 323 | UUUCUG | 635 |
| 001121 | | | 001743 | G_{F}^{S}A_{M}^{S}G_{F}G_{M}A_{F}A | | GGGAAA | | 001755 | U_{M}^{S}U_{F}^{S}U_{M}C_{F}U_{M} | | UUUUCCC | |
| | | | | _{M}G_{F}G_{M}G_{F}A_{M}A_{F}A | | ACAGAA | | | G_{F}U_{M}U_{F}U_{M}U_{F}C | | UUCCUCT | |
| | | | | _{M}A_{F}C_{M}A_{F}G_{M}A_{F}^{S}A | | A | | | _{M}C_{F}C_{M}U_{F}U_{M}C_{F}C | | T | |
| | | | | _{M}^{S}A_{F}-C7OH- | | | | | _{M}U_{F}C_{M}^{S}T_{D}^{S}T_{D}- | | | |
| | | | | [_{D}Tx-01-32] | | | | | OH-3′ | | | |
| DT- | 779 | 797 | DTS- | 5′-OH- | 690 | CCCAAAA | 1013 | DTS- | 5′-VP- | 324 | UUGAGU | 623 |
| 001122 | | | 001744 | C_{F}^{S}C_{M}^{S}C_{F}A_{M}A_{F}A | | UCCCAAA | | 001756 | U_{M}^{S}U_{F}^{S}G_{M}A_{F}G_{M} | | UUGGGA | |
| | | | | _{M}A_{F}U_{M}C_{F}C_{M}C_{F}A_{M} | | CUCAA | | | U_{F}U_{M}U_{F}G_{M}G_{F}G | | UUUUGG | |
| | | | | A_{F}A_{M}C_{F}U_{M}C_{F}^{S}A_{M} | | | | | _{M}A_{F}U_{M}U_{F}U_{M}U_{F} | | | |
| | | | | ^{S}A_{F}-C7OH- | | | | | G_{M}G_{F}G_{M}^{S}T_{D}^{S}T_{D}- | | | |
| | | | | [_{D}Tx-01-32] | | | | | OH-3′ | | GTT | |
| DT- | 785 | 803 | DTS- | 5′-OH- | 691 | AUCCCAA | 998 | DTS- | 5′-VP- | 325 | UUUGGU | 641 |
| 001123 | | | 001745 | A_{F}^{S}U_{M}^{S}C_{F}C_{M}C_{F}A | | ACUCAA | | 001757 | U_{M}^{S}U_{F}^{S}U_{M}G_{F}G_{M} | | UUGAGU | |
| | | | | _{M}A_{F}A_{M}C_{F}U_{M}C_{F}A | | ACCAAA | | | U_{F}U_{M}U_{F}G_{M}A_{F}G | | UUGGGA | |
| | | | | _{M}A_{F}A_{M}C_{F}C_{M}A_{F}^{S}A | | | | | _{M}U_{F}U_{M}U_{F}G_{M}G_{F} | | UTT | |
| | | | | _{M}^{S}A_{F}-C7OH- | | | | | G_{M}A_{F}U_{M}^{S}T_{D}^{S}T_{D}- | | | |
| | | | | [_{D}Tx-01-32] | | | | | OH-3′ | | | |
| DT- | 833 | 851 | DTS- | 5′-OH- | 692 | GCUGUU | 1062 | DTS- | 5′-VP- | 326 | UACAUC | 591 |
| 001124 | | | 001746 | G_{F}^{S}C_{M}^{S}U_{F}G_{M}U_{F}U | | GAUUGA | | 001758 | U_{M}^{S}A_{F}^{S}C_{M}A_{F}U_{M} | | UUCAAU | |
| | | | | _{M}G_{F}A_{M}U_{F}U_{M}G_{F}A | | AGAUGU | | | C_{F}U_{M}U_{F}C_{M}A_{F}A | | CAACAGC | |
| | | | | _{M}A_{F}G_{M}A_{F}U_{M}G_{F}^{S}U | | A | | | _{M}U_{F}C_{M}A_{F}A_{M}C_{F} | | TT | |
| | | | | _{M}^{S}A_{F}-C7OH- | | | | | A_{M}G_{F}C_{M}^{S}T_{D}^{S}T_{D}- | | | |
| | | | | [_{D}Tx-01-32] | | | | | OH-3′ | | | |
| DT- | 834 | 852 | DTS- | 5′-OH- | 693 | CUGUUG | 1043 | DTS- | 5′-VP- | 327 | AUACAU | 541 |
| 001125 | | | 001747 | C_{F}^{S}U_{M}^{S}G_{F}U_{M}U_{F}G | | AUUGAA | | 001759 | A_{M}^{S}U_{F}^{S}A_{M}C_{F}A_{M} | | CUUCAA | |
| | | | | _{M}A_{F}U_{M}U_{F}G_{M}A_{F}A | | GAUGUA | | | U_{F}C_{M}U_{F}U_{M}C_{F}A | | UCAACA | |
| | | | | _{M}G_{F}A_{M}U_{F}G_{M}U_{F}^{S}A | | U | | | _{M}A_{F}U_{M}C_{F}A_{M}A_{F} | | GTT | |
| | | | | _{M}^{S}U_{F}-C7OH- | | | | | C_{M}A_{F}G_{M}^{S}T_{D}^{S}T_{D}- | | | |
| | | | | [_{D}Tx-01-32] | | | | | OH-3′ | | | |
| DT- | 865 | 883 | DTS- | 5′-OH- | 694 | GUUUAU | 1080 | DTS- | 5′-VP- | 328 | AUAAAU | 539 |
| 001126 | | | 001748 | G_{F}^{S}U_{M}^{S}U_{F}U_{M}A_{F}U | | AAAACC | | 001760 | A_{M}^{S}U_{F}^{S}A_{M}A_{F}A_{M} | | AGGUUU | |
| | | | | _{M}A_{F}A_{M}A_{F}A_{M}C_{F}C | | UAUUUA | | | U_{F}A_{M}G_{F}G_{M}U_{F}U | | UAUAAA | |
| | | | | _{M}U_{F}A_{M}U_{F}U_{M}U_{F}^{S}A | | U | | | _{M}U_{F}U_{M}A_{F}U_{M}A_{F} | | CTT | |
| | | | | _{M}^{S}U_{F}-C7OH- | | | | | A_{M}A_{F}C_{M}^{S}T_{D}^{S}T_{D}- | | | |
| | | | | [_{D}Tx-01-32] | | | | | OH-3′ | | | |
| DT- | 904 | 922 | DTS- | 5′-OH- | 695 | ACAUAG | 985 | DTS- | 5′-VP- | 329 | AAAGCA | 494 |
| 001127 | | | 001749 | A_{F}^{S}C_{M}^{S}A_{F}U_{M}A_{F}G | | UAUUGU | | 001761 | A_{M}^{S}A_{F}^{S}A_{M}G_{F}C_{M} | | AACAAU | |
| | | | | _{M}U_{F}A_{M}U_{F}U_{M}G_{F}U | | UUGCUU | | | A_{F}A_{M}A_{F}C_{M}A_{F}A | | ACUAUG | |
| | | | | _{M}U_{F}U_{M}G_{F}C_{M}U_{F}^{S}U | | U | | | _{M}U_{F}A_{M}C_{F}U_{M}A_{F} | | UTT | |
| | | | | _{M}^{S}U_{F}-C7OH- | | | | | U_{M}G_{F}U_{M}^{S}T_{D}^{S}T_{D}- | | | |
| | | | | [_{D}Tx-01-32] | | | | | OH-3′ | | | |
| DT- | 929 | 947 | DTS- | 5′-OH- | 696 | UGACCA | 1095 | DTS- | 5′-VP- | 330 | AACACG | 497 |
| 001128 | | | 001750 | U_{F}^{S}G_{M}^{S}A_{F}C_{M}C_{F}A | | UCAGCCU | | 001762 | A_{M}^{S}A_{F}^{S}C_{M}A_{F}C_{M} | | AGGCUG | |
| | | | | _{M}U_{F}C_{M}A_{F}G_{M}C_{F}C_{M} | | CGUGUU | | | G_{F}A_{M}G_{F}G_{M}C_{F}U | | AUGGUC | |
| | | | | U_{F}C_{M}G_{F}U_{M}G_{F}^{S}U_{M} | | | | | _{M}G_{F}A_{M}U_{F}G_{M}G_{F} | | ATT | |
| | | | | ^{S}U_{F}-C7OH- | | | | | U_{M}C_{F}A_{M}^{S}T_{D}^{S}T_{D}- | | | |
| | | | | [_{D}Tx-01-32] | | | | | OH-3′ | | | |
| DT- | 950 | 968 | DTS- | 5′-OH- | 697 | GCCUUA | 1057 | DTS- | 5′-VP- | 331 | UUAGCU | 609 |
| 001129 | | | 001751 | G_{F}^{S}C_{M}^{S}C_{F}U_{M}U_{F}A | | AAGAAG | | 001763 | U_{M}^{S}U_{F}^{S}A_{M}G_{F}C_{M} | | ACUUCU | |
| | | | | _{M}A_{F}A_{M}G_{F}A_{M}A_{F}G | | UAGCUA | | | U_{F}A_{M}C_{F}U_{M}U_{F}C | | UUAAGG | |
| | | | | _{M}U_{F}A_{M}G_{F}C_{M}U_{F}^{S}A | | A | | | _{M}U_{F}U_{M}U_{F}A_{M}A_{F} | | CTT | |
| | | | | _{M}^{S}A_{F}-C7OH- | | | | | G_{M}G_{F}C_{M}^{S}T_{D}^{S}T_{D}- | | | |
| | | | | [_{D}Tx-01-32] | | | | | OH-3′ | | | |
| DT- | 958 | 976 | DTS- | 5′-OH- | 698 | GAAGUA | 1050 | DTS- | 5′-VP- | 332 | AAAGUU | 495 |
| 001130 | | | 001752 | G_{F}^{S}A_{M}^{S}A_{F}G_{M}U_{F}A | | GCUAAG | | 001764 | A_{M}^{S}A_{F}^{S}A_{M}G_{F}U_{M} | | CCUUAGC | |
| | | | | _{M}G_{F}C_{M}U_{F}A_{M}A_{F}G | | GAACUU | | | U_{F}C_{M}C_{F}U_{M}U_{F}A | | UACUUCT | |
| | | | | _{M}G_{F}A_{M}A_{F}C_{M}U_{F}^{S}U | | U | | | _{M}G_{F}C_{M}U_{F}A_{M}C_{F} | | T | |
| | | | | _{M}^{S}U_{F}-C7OH- | | | | | U_{M}U_{F}C_{M}^{S}T_{D}^{S}T_{D}- | | | |
| | | | | [_{D}Tx-01-32] | | | | | OH-3′ | | | |
| DT- | 967 | 985 | DTS- | 5′-OH- | 699 | AAGGAA | 982 | DTS- | 5′-VP- | 333 | UUAGGA | 610 |
| 001131 | | | 001753 | A_{F}^{S}A_{M}^{S}G_{F}G_{M}A_{F}A | | CUUUAC | | 001765 | U_{M}^{S}U_{F}^{S}A_{M}G_{F}G_{M} | | UGUAAA | |
| | | | | _{M}C_{F}U_{M}U_{F}U_{M}A_{F}C | | AUCCUA | | | A_{F}U_{M}G_{F}U_{M}A_{F}A | | GUUCCU | |
| | | | | _{M}A_{F}U_{M}C_{F}C_{M}U_{F}^{S}A | | A | | | _{M}A_{F}G_{M}U_{F}U_{M}C_{F} | | UTT | |
| | | | | _{M}^{S}A_{F}-C7OH- | | | | | C_{M}U_{F}U_{M}^{S}T_{D}^{S}T_{D}- | | | |
| | | | | [_{D}Tx-01-32] | | | | | OH-3′ | | | |
| DT- | 1786 | 1804 | DTS- | 5′-OH- | 700 | UGUGUG | 1101 | DTS- | 5′-VP- | 334 | AUGCAU | 558 |
| 001132 | | | 001754 | U_{F}^{S}G_{M}^{S}U_{F}G_{M}U_{F}G | | GACUAA | | 001766 | A_{M}^{S}U_{F}^{S}G_{M}C_{F}A_{M} | | CUUAGU | |
| | | | | _{M}G_{F}A_{M}C_{F}U_{M}A_{F}A | | GAUGCA | | | U_{F}C_{M}U_{F}U_{M}A_{F}G | | CCACACA | |
| | | | | _{M}G_{F}A_{M}U_{F}G_{M}C_{F}^{S}A | | U | | | _{M}U_{F}C_{M}C_{F}A_{M}C_{F} | | TT | |
| | | | | _{M}^{S}U_{F}-C7OH- | | | | | A_{M}C_{F}A_{M}^{S}T_{D}^{S}T_{D}- | | | |
| | | | | [_{D}Tx-01-32] | | | | | OH-3′ | | | |
| DT- | 737 | 755 | DTS- | 5′-OH- | 701 | GAGGAA | 1052 | DTS- | 5′-VP- | 323 | UUUCUG | 635 |
| 001145 | | | 001780 | G_{F}^{S}A_{M}^{S}G_{F}G_{M}A_{F}A | | GGGAAA | | 001755 | U_{M}^{S}U_{F}^{S}U_{M}C_{F}U_{M} | | UUUUCCC | |
| | | | | _{M}G_{F}G_{M}G_{F}A_{M}A_{F}A | | ACAGAA | | | G_{F}U_{M}U_{F}U_{M}U_{F}C | | UUCCUCT | |
| | | | | _{M}A_{F}C_{M}A_{F}G_{M}A_{F}^{S}A | | A | | | _{M}C_{F}C_{M}U_{F}U_{M}C_{F}C | | T | |
| | | | | _{M}^{S}A_{F}-C7OH- | | | | | _{M}U_{F}C_{M}^{S}T_{D}^{S}T_{D}- | | | |
| | | | | [_{D}Tx-01-08] | | | | | OH-3′ | | | |
| DT- | 779 | 797 | DTS- | 5′-OH- | 702 | CCCAAAA | 1013 | DTS- | 5′-VP- | 324 | UUGAGU | 623 |
| 001146 | | | 001781 | C_{F}^{S}C_{M}^{S}C_{F}A_{M}A_{F}A | | UCCCAAA | | 001756 | U_{M}^{S}U_{F}^{S}G_{M}A_{F}G_{M} | | UUGGGA | |
| | | | | _{M}A_{F}U_{M}C_{F}C_{M}C_{F}A_{M} | | CUCAA | | | U_{F}U_{M}U_{F}G_{M}G_{F}G | | UUUUGG | |
| | | | | A_{F}A_{M}C_{F}U_{M}C_{F}^{S}A_{M} | | | | | _{M}A_{F}U_{M}U_{F}U_{M}UR | | GTT | |
| | | | | ^{S}A_{F}-C7OH-[_{D}Tx- | | | | | G_{M}G_{F}G_{M}^{S}T_{D}^{S}T_{D}- | | | |
| | | | | 01-08] | | | | | OH-3′ | | | |
| DT- | 833 | 851 | DTS- | 5′-OH- | 703 | GCUGUU | 1062 | DTS- | 5′-VP- | 326 | UACAUC | 591 |
| 001147 | | | 001782 | G_{F}^{S}C_{M}^{S}U_{F}G_{M}U_{F}U | | GAUUGA | | 001758 | U_{M}^{S}A_{F}^{S}C_{M}A_{F}U_{M} | | UUCAAU | |
| | | | | _{M}G_{F}A_{M}U_{F}U_{M}G_{F}A | | AGAUGU | | | C_{F}U_{M}U_{F}C_{M}A_{F}A | | CAACAGC | |
| | | | | _{M}A_{F}G_{M}A_{F}U_{M}G_{F}^{S}U | | A | | | _{M}U_{F}C_{M}A_{F}A_{M}C_{F} | | TT | |
| | | | | _{M}^{S}A_{F}-C7OH- | | | | | A_{M}G_{F}C_{M}^{S}T_{D}^{S}T_{D}- | | | |
| | | | | [_{D}Tx-01-08] | | | | | OH-3′ | | | |
| DT- | 950 | 968 | DTS- | 5′-OH- | 704 | GCCUUA | 1057 | DTS- | 5′-VP- | 331 | UUAGCU | 609 |
| 001148 | | | 001783 | G_{F}^{S}C_{M}^{S}C_{F}U_{M}U_{F}A | | AAGAAG | | 001763 | U_{M}^{S}U_{F}^{S}A_{M}G_{F}C_{M} | | ACUUCU | |
| | | | | _{M}A_{F}A_{M}G_{F}A_{M}A_{F}G | | UAGCUA | | | U_{F}A_{M}C_{F}U_{M}U_{F}C | | UUAAGG | |
| | | | | _{M}U_{F}A_{M}G_{F}C_{M}U_{F}^{S}A | | A | | | _{M}U_{F}U_{M}U_{F}A_{M}A_{F} | | CTT | |
| | | | | _{M}^{S}A_{F}-C7OH- | | | | | G_{M}G_{F}C_{M}^{S}T_{D}^{S}T_{D}- | | | |
| | | | | [_{D}Tx-01-08] | | | | | OH-3′ | | | |
| DT- | 785 | 803 | DTS- | 5′-OH- | 705 | AUCCCAA | 998 | DTS- | 5′-VP- | 325 | UUUGGU | 641 |
| 001149 | | | 001784 | A_{F}^{S}U_{M}^{S}C_{F}C_{M}C_{F}A | | ACUCAA | | 001757 | U_{M}^{S}U_{F}^{S}U_{M}G_{F}G_{M} | | UUGAGU | |
| | | | | _{M}A_{F}A_{M}C_{F}U_{M}C_{F}A | | ACCAAA | | | U_{F}U_{M}U_{F}G_{M}A_{F}G | | UUGGGA | |
| | | | | _{M}A_{F}A_{M}C_{F}C_{M}A_{F}^{S}A | | | | | _{M}U_{F}U_{M}U_{F}G_{M}G_{F} | | UTT | |
| | | | | _{M}^{S}A_{F}-C7OH- | | | | | G_{M}A_{F}U_{M}^{S}T_{D}^{S}T_{D}- | | | |
| | | | | [_{D}Tx-01-08] | | | | | OH-3′ | | | |
| DT- | 834 | 852 | DTS- | 5′-OH- | 706 | CUGUUG | 1043 | DTS- | 5′-VP- | 327 | AUACAU | 541 |
| 001150 | | | 001785 | C_{F}^{S}U_{M}^{S}G_{F}U_{M}U_{F}G | | AUUGAA | | 001759 | A_{M}^{S}U_{F}^{S}A_{M}C_{F}A_{M} | | CUUCAA | |
| | | | | _{M}A_{F}U_{M}U_{F}G_{M}A_{F}A | | GAUGUA | | | U_{F}C_{M}U_{F}U_{M}C_{F}A | | UCAACA | |
| | | | | _{M}G_{F}A_{M}U_{F}G_{M}U_{F}^{S}A | | U | | | _{M}A_{F}U_{M}C_{F}A_{M}A_{F} | | GTT | |
| | | | | _{M}^{S}U_{F}-C7OH- | | | | | C_{M}A_{F}G_{M}^{S}T_{D}^{S}T_{D}- | | | |
| | | | | [_{D}Tx-01-08] | | | | | OH-3′ | | | |
| DT- | 865 | 883 | DTS- | 5′-OH- | 707 | GUUUAU | 1080 | DTS- | 5′-VP- | 328 | AUAAAU | 539 |
| 001151 | | | 001786 | G_{F}^{S}U_{M}^{S}U_{F}U_{M}A_{F}U | | AAAACC | | 001760 | A_{M}^{S}U_{F}^{S}A_{M}A_{F}A_{M} | | AGGUUU | |
| | | | | _{M}A_{F}A_{M}A_{F}A_{M}C_{F}C | | UAUUUA | | | U_{F}A_{M}G_{F}G_{M}U_{F}U | | UAUAAA | |
| | | | | _{M}U_{F}A_{M}U_{F}U_{M}U_{F}^{S}A | | U | | | _{M}U_{F}U_{M}A_{F}U_{M}A_{F} | | CTT | |
| | | | | _{M}^{S}U_{F}-C7OH- | | | | | A_{M}A_{F}C_{M}^{S}T_{D}^{S}T_{D}- | | | |
| | | | | [_{D}Tx-01-08] | | | | | OH-3′ | | | |
| DT- | 904 | 922 | DTS- | 5′-OH- | 708 | ACAUAG | 985 | DTS- | 5′-VP- | 329 | AAAGCA | 494 |
| 001152 | | | 001787 | A_{F}^{S}C_{M}^{S}A_{F}U_{M}A_{F}G | | UAUUGU | | 001761 | A_{M}^{S}A_{F}^{S}A_{M}G_{F}C_{M} | | AACAAU | |
| | | | | _{M}U_{F}A_{M}U_{F}U_{M}G_{F}U | | UUGCUU | | | A_{F}A_{M}A_{F}C_{M}A_{F}A | | ACUAUG | |
| | | | | _{M}U_{F}U_{M}G_{F}C_{M}U_{F}^{S}U | | U | | | _{M}U_{F}A_{M}C_{F}U_{M}A_{F} | | UTT | |
| | | | | _{M}^{S}U_{F}-C7OH- | | | | | U_{M}G_{F}U_{M}^{S}T_{D}^{S}T_{D}- | | | |
| | | | | [_{D}Tx-01-08] | | | | | OH-3′ | | | |
| DT- | 929 | 947 | DTS- | 5′-OH- | 709 | UGACCA | 1095 | DTS- | 5′-VP- | 330 | AACACG | 497 |
| 001153 | | | 001788 | U_{F}^{S}G_{M}^{S}A_{F}C_{M}C_{F}A | | UCAGCCU | | 001762 | A_{M}^{S}A_{F}^{S}C_{M}A_{F}C_{M} | | AGGCUG | |
| | | | | _{M}U_{F}C_{M}A_{F}G_{M}C_{F}C_{M} | | CGUGUU | | | G_{F}A_{M}G_{F}G_{M}C_{F}U | | AUGGUC | |
| | | | | U_{F}C_{M}G_{F}U_{M}G_{F}^{S}U_{M} | | | | | _{M}G_{F}A_{M}U_{F}G_{M}G_{F} | | ATT | |
| | | | | ^{S}U_{F}-C7OH-[_{D}Tx- | | | | | U_{M}C_{F}A_{M}^{S}T_{D}^{S}T_{D}- | | | |
| | | | | 01-08] | | | | | OH-3′ | | | |
| DT- | 958 | 976 | DTS- | 5′-OH- | 710 | GAAGUA | 1050 | DTS- | 5′-VP- | 332 | AAAGUU | 495 |
| 001154 | | | 001789 | G_{F}^{S}A_{M}^{S}A_{F}G_{M}U_{F}A | | GCUAAG | | 001764 | A_{M}^{S}A_{F}^{S}A_{M}G_{F}U_{M} | | CCUUAGC | |
| | | | | _{M}G_{F}C_{M}U_{F}A_{M}A_{F}G | | GAACUU | | | U_{F}C_{M}C_{F}U_{M}U_{F}A | | UACUUCT | |
| | | | | _{M}G_{F}A_{M}A_{F}C_{M}U_{F}^{S}U | | U | | | _{M}G_{F}C_{M}U_{F}A_{M}C_{F} | | T | |
| | | | | _{M}^{S}U_{F}-C7OH- | | | | | U_{M}U_{F}C_{M}^{S}T_{D}^{S}T_{D}- | | | |
| | | | | [_{D}Tx-01-08] | | | | | OH-3′ | | | |
| DT- | 967 | 985 | DTS- | 5′-OH- | 711 | AAGGAA | 982 | DTS- | 5′-VP- | 333 | UUAGGA | 610 |
| 001155 | | | 001790 | A_{F}^{S}A_{M}^{S}G_{F}G_{M}A_{F}A | | CUUUAC | | 001765 | U_{M}^{S}U_{F}^{S}A_{M}G_{F}G_{M} | | UGUAAA | |
| | | | | _{M}C_{F}U_{M}U_{F}U_{M}A_{F}C | | AUCCUA | | | A_{F}U_{M}G_{F}U_{M}A_{F}A | | GUUCCU | |
| | | | | _{M}A_{F}U_{M}C_{F}C_{M}U_{F}^{S}A | | A | | | _{M}A_{F}G_{M}U_{F}U_{M}C_{F} | | UTT | |
| | | | | _{M}^{S}A_{F}-C7OH- | | | | | C_{M}U_{F}U_{M}^{S}T_{D}^{S}T_{D}- | | | |
| | | | | [_{D}Tx-01-08] | | | | | OH-3′ | | | |
| DT- | 1786 | 1804 | DTS- | 5′-OH- | 712 | UGUGUG | 1101 | DTS- | 5′-VP- | 334 | AUGCAU | 558 |
| 001156 | | | 001791 | U_{F}^{S}G_{M}^{S}U_{F}G_{M}U_{F}G | | GACUAA | | 001766 | A_{M}^{S}U_{F}^{S}G_{M}C_{F}A_{M} | | CUUAGU | |
| | | | | _{M}G_{F}A_{M}C_{F}U_{M}A_{F}A | | GAUGCA | | | U_{F}C_{M}U_{F}U_{M}A_{F}G | | CCACACA | |
| | | | | _{M}G_{F}A_{M}U_{F}G_{M}C_{F}^{S}A | | U | | | _{M}U_{F}C_{M}C_{F}A_{M}C_{E} | | TT | |
| | | | | _{M}^{S}U_{F}-C7OH- | | | | | A_{M}C_{F}A_{M}^{S}T_{D}^{S}T_{D}- | | | |
| | | | | [_{D}Tx-01-08] | | | | | OH-3′ | | | |
| DT- | 474 | 492 | DTS- | 5′-OH- | 713 | GGCUCU | 1068 | DTS- | 5′-PO4- | 317 | GAAGAA | 580 |
| 001157 | | | 001792 | G_{F}^{S}G_{M}^{S}C_{F}U_{M}C_{F}U | | GUUCCU | | 001732 | G_{M}^{S}A_{F}^{S}A_{M}G_{F}A_{M} | | CAGGAA | |
| | | | | _{M}G_{F}U_{M}U_{F}C_{M}C_{F}U | | GUUCUU | | | A_{F}C_{M}A_{F}G_{M}G_{F}A | | CAGAGCC | |
| | | | | _{M}G_{F}U_{M}U_{F}C_{M}U_{F}^{S}U | | C | | | _{M}A_{F}C_{M}A_{F}G_{M}A_{F} | | TT | |
| | | | | _{M}^{S}U_{F}-C7OH- | | | | | G_{M}C_{F}C_{M}^{S}T_{D}^{S}T_{D}- | | | |
| | | | | [_{D}Tx-01-08] | | | | | OH-3′ | | | |
| DT- | 874 | 892 | DTS- | 5′-OH- | 714 | ACCUAU | 988 | DTS- | 5′-PO4- | 318 | AAAAGU | 491 |
| 001158 | | | 001793 | A_{F}^{S}C_{M}^{S}C_{F}U_{M}A_{F}U | | UUAUAA | | 001734 | A_{M}^{S}A_{F}^{S}A_{M}A_{F}G_{M} | | GUUAUA | |
| | | | | _{M}U_{F}U_{M}A_{F}U_{M}A_{F}A | | CACUUU | | | U_{F}G_{M}U_{F}U_{M}A_{F}U | | AAUAGG | |
| | | | | _{M}C_{F}A_{M}C_{F}U_{M}U_{F}^{S}U | | U | | | _{M}A_{F}A_{M}A_{F}U_{M}A_{F} | | UTT | |
| | | | | _{M}^{S}U_{F}-C7OH- | | | | | G_{M}G_{F}U_{M}^{S}T_{D}^{S}T_{D}- | | | |
| | | | | [_{D}Tx-01-08] | | | | | OH-3′ | | | |
| DT- | 1562 | 1680 | DTS- | 5′-OH- | 715 | ACAAUA | 984 | DTS- | 5′-PO4- | 319 | UUUGAG | 637 |
| 001159 | | | 001794 | A_{F}^{S}C_{M}^{S}A_{F}A_{M}U_{F}A | | AAUAAA | | 001736 | U_{M}^{S}U_{F}^{S}U_{M}G_{F}A_{M} | | AUUUAU | |
| | | | | _{M}A_{F}A_{M}U_{F}A_{M}A_{F}A | | UCUCAA | | | G_{F}A_{M}U_{F}U_{M}U_{F}A | | UUAUUG | |
| | | | | _{M}U_{F}C_{M}U_{F}C_{M}A_{F}^{S}A | | A | | | _{M}U_{F}U_{M}U_{F}A_{M}U_{F} | | UTT | |
| | | | | _{M}^{S}A_{F}-C7OH- | | | | | U_{M}G_{F}U_{M}^{S}T_{D}^{S}T_{D}- | | | |
| | | | | [_{D}Tx-01-08] | | | | | OH-3′ | | | |
| DT- | 989 | 1007 | DTS- | 5′-OH- | 716 | CCUCGUG | 1020 | DTS- | 5′-PO4- | 320 | UUUAAG | 630 |
| 001160 | | | 001795 | C_{F}^{S}C_{M}^{S}U_{F}C_{M}G_{F}U | | UUGAAU | | 001738 | U_{M}^{S}U_{F}^{S}U_{M}A_{F}A_{M} | | AUUCAA | |
| | | | | _{M}G_{F}U_{M}U_{F}G_{M}A_{F}A | | CUUAAA | | | G_{F}A_{M}U_{F}U_{M}C_{F}A | | CACGAG | |
| | | | | _{M}U_{F}C_{M}U_{F}U_{M}A_{F}^{S}A | | | | | _{M}A_{F}C_{M}A_{F}C_{M}G_{F} | | GTT | |
| | | | | _{M}^{S}A_{F}-C7OH- | | | | | A_{M}G_{F}G_{M}^{S}T_{D}^{S}T_{D}- | | | |
| | | | | [_{D}Tx-01-08] | | | | | OH-3′ | | | |
| DT- | 1695 | 1713 | DTS- | 5′-OH- | 717 | CCACCAA | 1011 | DTS- | 5′-PO4- | 267 | AUACAU | 540 |
| 001161 | | | 001796 | C_{F}^{S}C_{M}^{S}A_{F}C_{M}C_{F}A | | CUGUAG | | 001420 | A_{M}^{S}U_{F}^{S}A_{M}C_{F}A_{M} | | CUACAG | |
| | | | | _{M}A_{F}C_{M}U_{F}G_{M}U_{F}A | | AUGUAU | | | U_{F}C_{M}U_{F}A_{M}C_{F}A | | UUGGUG | |
| | | | | _{M}G_{F}A_{M}U_{F}G_{M}U_{F}^{S}A | | | | | _{M}G_{F}U_{M}U_{F}G_{M}G_{F} | | GTT | |
| | | | | _{M}^{S}U_{F}-C7OH- | | | | | U_{M}G_{F}G_{M}^{S}T_{D}^{S}T_{D}- | | | |
| | | | | [_{D}Tx-01-08] | | | | | OH-3′ | | | |
| DT- | 398 | 416 | DTS- | 5′-OH- | 718 | CUGUCCA | 1042 | DTS- | 5′-PO4- | 170 | AUCAUG | 548 |
| 001162 | | | 001797 | C_{F}^{S}U_{M}^{S}G_{F}U_{M}C_{F}C | | GGCCACC | | 000605 | A_{M}^{S}U_{F}^{S}C_{M}A_{F}U_{M} | | GUGGCC | |
| | | | | _{M}A_{F}G_{M}G_{F}C_{M}C_{F}A | | AUGAU | | | G_{F}G_{M}U_{F}G_{M}G_{F}C | | UGGACA | |
| | | | | _{M}C_{F}C_{M}A_{F}U_{M}G_{F}^{S}A | | | | | _{M}C_{F}U_{M}G_{F}G_{M}A_{F} | | GTT | |
| | | | | _{M}U_{F}-C7OH- | | | | | C_{M}A_{F}G_{M}^{S}T_{D}^{S}T_{D}- | | | |
| | | | | [_{D}Tx-01-08] | | | | | OH-3′ | | | |
| DT- | 1785 | 1803 | DTS- | 5′-OH- | 719 | AUACCA | 995 | DTS- | 5′-PO4- | 321 | UAGUCC | 597 |
| 001163 | | | 001798 | A_{F}^{S}U_{M}^{S}A_{F}C_{M}C_{F}A | | ACUGUG | | 001740 | U_{M}^{S}A_{F}^{S}G_{M}U_{F}C_{M} | | ACACAG | |
| | | | | _{M}A_{F}C_{M}U_{F}G_{M}U_{F}G | | UGGACU | | | C_{F}A_{M}C_{F}A_{M}C_{F}A | | UUGGUA | |
| | | | | _{M}U_{F}G_{M}G_{F}A_{M}C_{F}^{S}U | | A | | | _{M}G_{F}U_{M}U_{F}G_{M}G_{F} | | UTT | |
| | | | | _{M}^{S}A_{F}-C7OH- | | | | | U_{M}A_{F}U_{M}^{S}T_{D}^{S}T_{D}- | | | |
| | | | | [_{D}Tx-01-08] | | | | | OH-3′ | | | |
| DT- | 872 | 890 | DTS- | 5′-OH- | 720 | AAACCU | 976 | DTS- | 5′-PO4- | 322 | AAGUGU | 510 |
| 001164 | | | 001799 | A_{F}^{S}A_{M}^{S}A_{F}C_{M}C_{F}U | | AUUUAU | | 001742 | A_{M}^{S}A_{F}^{S}G_{M}U_{F}G_{M} | | UAUAAA | |
| | | | | _{M}A_{F}U_{M}U_{F}U_{M}A_{F}U | | AACACU | | | U_{F}U_{M}A_{F}U_{M}A_{F}A | | UAGGUU | |
| | | | | _{M}A_{F}A_{M}C_{F}A_{M}C_{F}^{S}U | | U | | | _{M}A_{F}U_{M}A_{F}G_{M}G_{F} | | UTT | |
| | | | | _{M}^{S}U_{F}-C7OH- | | | | | U_{M}U_{F}U_{M}^{S}T_{D}^{S}T_{D}- | | | |
| | | | | [_{D}Tx-01-08] | | | | | OH-3′ | | | |
| DT- | 214 | 232 | DTS- | 5′-OH- | 721 | CUCCUGU | 1036 | DTS- | 5′-PO4- | 192 | UGAUAC | 605 |
| 001176 | | | 001813 | C_{F}^{S}U_{M}^{S}C_{F}C_{M}U_{F}G | | UGCUGA | | 001270 | U_{M}^{S}G_{F}^{S}A_{M}U_{F}A_{M} | | UCAGCA | |
| | | | | _{M}U_{F}U_{M}G_{F}C_{M}U_{F}G | | GUAUCA | | | C_{F}U_{M}C_{F}A_{M}G_{F}C | | ACAGGA | |
| | | | | _{M}A_{F}G_{M}U_{F}A_{M}U_{F}^{S}C | | | | | _{M}A_{F}A_{M}C_{F}A_{M}G_{F} | | GTT | |
| | | | | _{M}^{S}A_{F}-C7OH- | | | | | G_{M}A_{F}G_{M}^{S}T_{D}^{S}T_{D}- | | | |
| | | | | [_{D}Tx-01-08] | | | | | OH-3′ | | | |
| DT- | 217 | 235 | DTS- | 5′-OH- | 722 | CUGUUG | 1044 | DTS- | 5′-PO4- | 194 | CGAUGA | 579 |
| 001177 | | | 001814 | C_{F}^{S}U_{M}^{S}G_{F}U_{M}U_{F}G | | CUGAGU | | 001274 | C_{M}^{S}G_{F}^{S}A_{M}U_{F}G_{M} | | UACUCA | |
| | | | | _{M}C_{F}U_{M}G_{F}A_{M}G_{F}U | | AUCAUC | | | A_{F}U_{M}A_{F}C_{M}U_{F}C | | GCAACA | |
| | | | | _{M}A_{F}U_{M}C_{F}A_{M}U_{F}^{S}C | | G | | | _{M}A_{F}G_{M}C_{F}A_{M}A_{F} | | GTT | |
| | | | | _{M}^{S}G_{F}-C7OH- | | | | | C_{M}A_{F}G_{M}^{S}T_{D}^{S}T_{D}- | | | |
| | | | | [_{D}Tx-01-08] | | | | | OH-3′ | | | |
| DT- | 219 | 237 | DTS- | 5′-OH- | 723 | GUUGCU | 1079 | DTS- | 5′-PO4- | 196 | GACGAU | 581 |
| 001178 | | | 001815 | G_{F}^{S}U_{M}^{S}U_{F}G_{M}C_{F}U | | GAGUAU | | 001278 | G_{M}^{S}A_{F}^{S}C_{M}G_{F}A_{M} | | GAUACU | |
| | | | | _{M}G_{F}A_{M}G_{F}U_{M}A_{F}U | | CAUCGUC | | | U_{F}G_{M}A_{F}U_{M}A_{F}C | | CAGCAAC | |
| | | | | _{M}C_{F}A_{M}U_{F}C_{M}G_{F}^{S}U | | | | | _{M}U_{F}C_{M}A_{F}G_{M}C_{F} | | TT | |
| | | | | _{M}^{S}U_{F}-C7OH- | | | | | A_{M}A_{F}C_{M}^{S}T_{D}^{S}T_{D}- | | | |
| | | | | [_{D}Tx-01-08] | | | | | OH-3′ | | | |
| DT- | 227 | 245 | DTS- | 5′-OH- | 724 | GUAUCA | 1074 | DTS- | 5′-PO4- | 199 | ACGUGG | 524 |
| 001179 | | | 001816 | G_{F}^{S}U_{M}^{S}A_{F}U_{M}C_{F}A | | UCGUCCU | | 001284 | A_{M}^{S}C_{F}^{S}G_{M}U_{F}G_{M} | | AGGACG | |
| | | | | _{M}U_{F}C_{M}G_{F}U_{M}C_{F}C_{M} | | CCACGU | | | G_{F}A_{M}G_{F}G_{M}A_{F}C | | AUGAUA | |
| | | | | U_{F}C_{M}C_{F}A_{M}C_{F}^{S}G_{M} | | | | | _{M}G_{F}A_{M}U_{F}G_{M}A_{F} | | CTT | |
| | | | | ^{S}U_{F}-C7OH-[_{D}Tx- | | | | | U_{M}A_{F}C_{M}^{S}T_{D}^{S}T_{D}- | | | |
| | | | | 01-08] | | | | | OH-3′ | | | |
| DT- | 245 | 263 | DTS- | 5′-OH- | 725 | UCGCGG | 1092 | DTS- | 5′-PO4- | 200 | AGCAGC | 534 |
| 001180 | | | 001817 | U_{F}^{S}C_{M}^{S}G_{F}C_{M}G_{F}G | | UGCUGG | | 001286 | A_{M}^{S}G_{F}^{S}C_{M}A_{F}G_{M} | | ACCAGCA | |
| | | | | _{M}U_{F}G_{M}C_{F}U_{M}G_{F}G | | UGCUGC | | | C_{F}A_{M}C_{F}C_{M}A_{F}G | | CCGCGAT | |
| | | | | _{M}U_{F}G_{M}C_{F}U_{M}G_{F}^{S}C | | U | | | _{M}C_{F}A_{M}C_{F}C_{M}G_{F}C | | T | |
| | | | | ^{S}U_{F}-C7OH- | | | | | _{M}G_{F}A_{M}^{S}T_{D}^{S}T_{D}- | | | |
| | | | | [_{D}Tx-01-08] | | | | | OH-3′ | | | |
| DT- | 253 | 271 | DTS- | 5′-OH- | 726 | CUGGUG | 1041 | DTS- | 5′-PO4- | 202 | AGACGA | 531 |
| 001181 | | | 001818 | C_{F}^{S}U_{M}^{S}G_{F}G_{M}U_{F}G | | CUGCUG | | 001290 | A_{M}^{S}G_{F}^{S}A_{M}C_{F}G_{M} | | ACAGCA | |
| | | | | _{M}C_{F}U_{M}G_{F}C_{M}U_{F}G | | UUCGUC | | | A_{F}A_{M}C_{F}A_{M}G_{F}C | | GCACCAG | |
| | | | | _{M}U_{F}U_{M}C_{F}G_{M}U_{F}^{S}C | | U | | | _{M}A_{F}G_{M}C_{F}A_{M}C_{F} | | TT | |
| | | | | _{M}^{S}U_{F}-C7OH- | | | | | C_{M}A_{F}G_{M}^{S}T_{D}^{S}T_{D}- | | | |
| | | | | [_{D}Tx-01-08] | | | | | OH-3′ | | | |
| DT- | 304 | 322 | DTS- | 5′-OH- | 727 | GGACAC | 1064 | DTS- | 5′-PO4- | 210 | AGAGAU | 532 |
| 001182 | | | 001819 | G_{F}^{S}G_{M}^{S}A_{F}C_{M}A_{F}C | | GCAACU | | 001306 | A_{M}^{S}G_{F}^{S}A_{M}G_{F}A_{M} | | CAGUUG | |
| | | | | _{M}G_{F}C_{M}A_{F}A_{M}C_{F}U | | GAUCUC | | | U_{F}C_{M}A_{F}G_{M}U_{F}U | | CGUGUCC | |
| | | | | _{M}G_{F}A_{M}U_{F}C_{M}U_{F}^{S}C | | U | | | _{M}G_{F}C_{M}G_{F}U_{M}G_{F} | | TT | |
| | | | | _{M}^{S}U_{F}-C7OH- | | | | | U_{M}C_{F}C_{M}^{S}T_{D}^{S}T_{D}- | | | |
| | | | | [_{D}Tx-01-08] | | | | | OH-3′ | | | |
| DT- | 350 | 368 | DTS- | 5′-OH- | 728 | GAAAUG | 1049 | DTS- | 5′-PO4- | 212 | AAACAG | 492 |
| 001183 | | | 001820 | G_{F}^{S}A_{M}^{S}A_{F}A_{M}U_{F}G | | UCCACCA | | 001310 | A_{M}^{S}A_{F}^{S}A_{M}C_{F}A_{M} | | UGGUGG | |
| | | | | _{M}U_{F}C_{M}C_{F}A_{M}C_{F}C_{M} | | CUGUUU | | | G_{F}U_{M}G_{F}G_{M}U_{F}G | | ACAUUU | |
| | | | | A_{F}C_{M}U_{F}G_{M}U_{F}^{S}U_{M} | | | | | _{M}G_{F}A_{M}C_{F}A_{M}U_{F} | | CTT | |
| | | | | ^{S}U_{F}-C7OH-[_{D}Tx- | | | | | U_{M}U_{F}C_{M}^{S}T_{D}^{S}T_{D}- | | | |
| | | | | 01-08] | | | | | OH-3′ | | | |
| DT- | 358 | 376 | DTS- | 5′-OH- | 729 | CACCACU | 1005 | DTS- | 5′-PO4- | 213 | AUGAUG | 556 |
| 001184 | | | 001821 | C_{F}^{S}A_{M}^{S}C_{F}C_{M}A_{F}C | | GUUUCU | | 001312 | A_{M}^{S}U_{F}^{S}G_{M}A_{F}U_{M} | | AGAAAC | |
| | | | | _{M}U_{F}G_{M}U_{F}U_{M}U_{F}C | | CAUCAU | | | G_{F}A_{M}G_{F}A_{M}A_{F}A | | AGUGGU | |
| | | | | _{M}U_{F}C_{M}A_{F}U_{M}C_{F}^{S}A | | | | | _{M}C_{F}A_{M}G_{F}U_{M}G_{F} | | GTT | |
| | | | | _{M}^{S}U_{F}-C7OH- | | | | | G_{M}U_{F}G_{M}^{S}T_{D}^{S}T_{D}- | | | |
| | | | | [_{D}Tx-01-08] | | | | | OH-3′ | | | |
| DT- | 383 | 401 | DTS- | 5′-OH- | 730 | ACGAAU | 989 | DTS- | 5′-PO4- | 216 | ACAGAC | 518 |
| 001185 | | | 001822 | A_{F}^{S}C_{M}^{S}G_{F}A_{M}A_{F}U | | GGCUGC | | 001318 | A_{M}^{S}C_{F}^{S}A_{M}G_{F}A_{M} | | UGCAGCC | |
| | | | | _{M}G_{F}G_{M}C_{F}U_{M}G_{F}C | | AGUCUG | | | C_{F}U_{M}G_{F}C_{M}A_{F}G | | AUUCGUT | |
| | | | | _{M}A_{F}G_{M}U_{F}C_{M}U_{F}^{S}G | | U | | | _{M}C_{F}C_{M}A_{F}U_{M}U_{F} | | T | |
| | | | | _{M}^{S}U_{F}-C7OH- | | | | | C_{M}G_{F}U_{M}^{S}T_{D}^{S}T_{D}- | | | |
| | | | | [_{D}Tx-01-08] | | | | | OH-3′ | | | |
| DT- | 452 | 470 | DTS- | 5′-OH- | 731 | UGUUCU | 1102 | DTS- | 5′-PO4- | 221 | AAGAGU | 503 |
| 001186 | | | 001823 | U_{F}^{S}G_{M}^{S}U_{F}U_{M}C_{F}U | | UCUGCCA | | 001328 | A_{M}^{S}A_{F}^{S}G_{M}A_{F}G_{M} | | UGGCAG | |
| | | | | _{M}U_{F}C_{M}U_{F}G_{M}C_{F}C_{M} | | ACUCUU | | | U_{F}U_{M}G_{F}G_{M}C_{F}A | | AAGAAC | |
| | | | | A_{F}A_{M}C_{F}U_{M}C_{F}^{S}U_{M} | | | | | _{M}G_{F}A_{M}A_{F}G_{M}A_{F} | | ATT | |
| | | | | ^{S}U_{F}-C7OH-[_{D}Tx- | | | | | A_{M}C_{F}A_{M}^{S}T_{D}^{S}T_{D}- | | | |
| | | | | 01-08] | | | | | OH-3′ | | | |
| DT- | 162 | 180 | DTS- | 5′-OH- | 732 | CUGUUU | 1046 | DTS- | 5′-PO4- | 293 | UUUCUG | 633 |
| 001187 | | | 001824 | C_{F}^{S}U_{M}^{S}G_{F}U_{M}U_{F}U | | GGCCGG | | 001604 | U_{M}^{S}U_{F}^{S}U_{M}C_{F}U_{M} | | CCCGGCC | |
| | | | | _{M}G_{F}G_{M}C_{F}C_{M}G_{F}G | | GCAGAA | | | G_{F}C_{M}C_{F}C_{M}G_{F}G | | AAACAGT | |
| | | | | _{M}G_{F}C_{M}A_{F}G_{M}A_{F}^{S}A | | A | | | _{M}C_{F}C_{M}A_{F}A_{M}A_{F} | | T | |
| | | | | _{M}^{S}A_{F}-C7OH- | | | | | C_{M}A_{F}G_{M}^{S}T_{D}^{S}T_{D}- | | | |
| | | | | [_{D}Tx-01-08] | | | | | OH-3′ | | | |
| DT- | 177 | 195 | DTS- | 5′-OH- | 733 | GAAACU | 1048 | DTS- | 5′-PO4- | 294 | UUCUGC | 618 |
| 001188 | | | 001825 | G_{F}^{S}A_{M}^{S}A_{F}A_{M}C_{F}U | | CCGCUGA | | 001606 | U_{M}^{S}U_{F}^{S}C_{M}U_{F}G_{M} | | UCAGCG | |
| | | | | _{M}C_{F}C_{M}G_{F}C_{M}U_{F}G_{M} | | GCAGAA | | | C_{F}U_{M}C_{F}A_{M}G_{F}C | | GAGUUU | |
| | | | | A_{F}G_{M}C_{F}A_{M}G_{F}^{S}A_{M} | | | | | _{M}G_{F}G_{M}A_{F}G_{M}U_{F} | | CTT | |
| | | | | ^{S}A_{F}-C7OH- | | | | | U_{M}U_{F}C_{M}^{S}T_{D}^{S}T_{D}- | | | |
| | | | | [_{D}Tx-01-08] | | | | | OH-3′ | | | |
| DT- | 180 | 198 | DTS- | 5′-OH- | 734 | ACUCCGC | 991 | DTS- | 5′-PO4- | 295 | AAGUUC | 511 |
| 001189 | | | 001826 | A_{F}^{S}C_{M}^{S}U_{F}C_{M}C_{F}G | | UGAGCA | | 001608 | A_{M}^{S}A_{F}^{S}G_{M}U_{F}U_{M} | | UGCUCA | |
| | | | | _{M}C_{F}U_{M}G_{F}A_{M}G_{F}C | | GAACUU | | | C_{F}U_{M}G_{F}C_{M}U_{F}C | | GCGGAG | |
| | | | | _{M}A_{F}G_{M}A_{F}A_{M}C_{F}^{S}U | | | | | _{M}A_{F}G_{M}C_{F}G_{M}G_{F} | | UTT | |
| | | | | _{M}^{S}U_{F}-C7OH- | | | | | A_{M}G_{F}U_{M}^{S}T_{D}^{S}T_{D}- | | | |
| | | | | [_{D}Tx-01-08] | | | | | OH-3′ | | | |
| DT- | 217 | 235 | DTS- | 5′-OH- | 722 | CUGUUG | 1044 | DTS- | 5′-VP- | 335 | CGAUGA | 579 |
| 001190 | | | 001814 | C_{F}^{S}U_{M}^{S}G_{F}U_{M}U_{F}G | | CUGAGU | | 001827 | C_{M}^{S}G_{F}^{S}A_{M}U_{F}G_{M} | | UACUCA | |
| | | | | _{M}C_{F}U_{M}G_{F}A_{M}G_{F}U | | AUCAUC | | | A_{F}U_{M}A_{F}C_{M}U_{F}C | | GCAACA | |
| | | | | _{M}A_{F}U_{M}C_{F}A_{M}U_{F}^{S}C | | G | | | _{M}A_{F}G_{M}C_{F}A_{M}A_{F} | | GTT | |
| | | | | _{M}^{S}G_{F}-C7OH- | | | | | C_{M}A_{F}G_{M}^{S}T_{D}^{S}T_{D}- | | | |
| | | | | [_{D}Tx-01-08] | | | | | OH-3′ | | | |
| DT- | 219 | 237 | DTS- | 5′-OH- | 723 | GUUGCU | 1079 | DTS- | 5′-VP- | 336 | GACGAU | 581 |
| 001191 | | | 001815 | G_{F}^{S}U_{M}^{S}U_{F}G_{M}C_{F}U | | GAGUAU | | 001828 | G_{M}^{S}A_{F}^{S}C_{M}G_{F}A_{M} | | GAUACU | |
| | | | | _{M}G_{F}A_{M}G_{F}U_{M}A_{F}U | | CAUCGUC | | | U_{F}G_{M}A_{F}U_{M}A_{F}C | | CAGCAAC | |
| | | | | _{M}C_{F}A_{M}U_{F}C_{M}G_{F}^{S}U | | | | | _{M}U_{F}C_{M}A_{F}G_{M}C_{F} | | TT | |
| | | | | _{M}^{S}U_{F}-C7OH- | | | | | A_{M}A_{F}C_{M}^{S}T_{D}^{S}T_{D}- | | | |
| | | | | [_{D}Tx-01-08] | | | | | OH-3′ | | | |
| DT- | 227 | 245 | DTS- | 5′-OH- | 724 | GUAUCA | 1074 | DTS- | 5′-VP- | 337 | ACGUGG | 524 |
| 001192 | | | 001816 | G_{F}^{S}U_{M}^{S}A_{F}U_{M}C_{F}A | | UCGUCCU | | 001829 | A_{M}^{S}C_{F}^{S}G_{M}U_{F}G_{M} | | AGGACG | |
| | | | | _{M}U_{F}C_{M}G_{F}U_{M}C_{F}C_{M} | | CCACGU | | | G_{F}A_{M}G_{F}G_{M}A_{F}C | | AUGAUA | |
| | | | | U_{F}C_{M}C_{F}A_{M}C_{F}^{S}G_{M} | | | | | _{M}G_{F}A_{M}U_{F}G_{M}A_{F} | | CTT | |
| | | | | ^{S}U_{F}-C7OH-[_{D}Tx- | | | | | U_{M}A_{F}C_{M}^{S}T_{D}^{S}T_{D}- | | | |
| | | | | 01-08] | | | | | OH-3′ | | | |
| DT- | 245 | 263 | DTS- | 5′-OH- | 725 | UCGCGG | 1092 | DTS- | 5′-VP- | 338 | AGCAGC | 534 |
| 001193 | | | 001817 | U_{F}^{S}C_{M}^{S}G_{F}C_{M}G_{F}G | | UGCUGG | | 001830 | A_{M}^{S}G_{F}^{S}C_{M}A_{F}G_{M} | | ACCAGCA | |
| | | | | _{M}U_{F}G_{M}C_{F}U_{M}G_{F}G | | UGCUGC | | | C_{F}A_{M}C_{F}C_{M}A_{F}G | | CCGCGAT | |
| | | | | _{M}U_{F}G_{M}C_{F}U_{M}G_{F}^{S}C | | U | | | _{M}C_{F}A_{M}C_{F}C_{M}G_{F}C | | T | |
| | | | | _{M}^{S}U_{F}-C7OH- | | | | | _{M}G_{F}A_{M}^{S}T_{D}^{S}T_{D}- | | | |
| | | | | [_{D}Tx-01-08] | | | | | OH-3′ | | | |
| DT- | 253 | 271 | DTS- | 5′-OH- | 726 | CUGGUG | 1041 | DTS- | 5′-VP- | 339 | AGACGA | 531 |
| 001194 | | | 001818 | C_{F}^{S}U_{M}^{S}G_{F}G_{M}U_{F}G | | CUGCUG | | 001831 | A_{M}^{S}G_{F}^{S}A_{M}C_{F}G_{M} | | ACAGCA | |
| | | | | _{M}C_{F}U_{M}G_{F}C_{M}U_{F}G | | UUCGUC | | | A_{F}A_{M}C_{F}A_{M}G_{F}C | | GCACCAG | |
| | | | | _{M}U_{F}U_{M}C_{F}G_{M}U_{F}^{S}C | | U | | | _{M}A_{F}G_{M}C_{F}A_{M}C_{E} | | TT | |
| | | | | _{M}^{S}U_{F}-C7OH- | | | | | C_{M}A_{F}G_{M}^{S}T_{D}^{S}T_{D}- | | | |
| | | | | [_{D}Tx-01-08] | | | | | OH-3′ | | | |
| DT- | 464 | 482 | DTS- | 5′-OH- | 735 | AACUCU | 980 | DTS- | 5′-PO4- | 223 | UUGGUG | 628 |
| 001195 | | | 001832 | A_{F}^{S}A_{M}^{S}C_{F}U_{M}C_{F}U | | UCACCCU | | 001332 | U_{M}^{S}U_{F}^{S}G_{M}G_{F}U_{M} | | AGGGUG | |
| | | | | _{M}U_{F}C_{M}A_{F}C_{M}C_{F}C_{M} | | CACCAA | | | G_{F}A_{M}G_{F}G_{M}G_{F}U | | AAGAGU | |
| | | | | U_{F}C_{M}A_{F}C_{M}C_{F}^{S}A_{M} | | | | | _{M}G_{F}A_{M}A_{F}G_{M}A_{F} | | UTT | |
| | | | | ^{S}A_{F}-C7OH- | | | | | G_{M}U_{F}U_{M}^{S}T_{D}^{S}T_{D}- | | | |
| | | | | [_{D}Tx- 01-08] | | | | | OH-3′ | | | |
| DT- | 486 | 504 | DTS- | 5′-OH- | 736 | GGGCAG | 1070 | DTS- | 5′-PO4- | 224 | AGUGAU | 536 |
| 001196 | | | 001833 | G_{F}^{S}G_{M}^{S}G_{F}C_{M}A_{F}G | | GUUUUA | | 001334 | A_{M}^{S}G_{F}^{S}U_{M}G_{F}A_{M} | | GUAAAA | |
| | | | | _{M}G_{F}U_{M}U_{F}U_{M}U_{F}A | | CAUCACU | | | U_{F}G_{M}U_{F}A_{M}A_{F}A | | CCUGCCC | |
| | | | | _{M}C_{F}A_{M}U_{F}C_{M}A_{F}^{S}C | | | | | _{M}A_{F}C_{M}C_{F}U_{M}G_{F} | | TT | |
| | | | | _{M}^{S}U_{F}-C7OH- | | | | | C_{M}C_{F}C_{M}^{S}T_{D}^{S}T_{D}- | | | |
| | | | | [_{D}Tx-01-08] | | | | | OH-3′ | | | |
| DT- | 491 | 509 | DTS- | 5′-OH- | 737 | GGUUUU | 1072 | DTS- | 5′-PO4- | 225 | AUUCCA | 567 |
| 001197 | | | 001834 | G_{F}^{S}G_{M}^{S}U_{F}U_{M}U_{F}U | | ACAUCAC | | 001336 | A_{M}^{S}U_{F}^{S}U_{M}C_{F}C_{M} | | GUGAUG | |
| | | | | _{M}A_{F}C_{M}A_{F}U_{M}C_{F}A | | UGGAAU | | | A_{F}G_{M}U_{F}G_{M}A_{F}U | | UAAAAC | |
| | | | | _{M}C_{F}U_{M}G_{F}G_{M}A_{F}^{S}A | | | | | _{M}G_{F}U_{M}A_{F}A_{M}A_{F} | | CTT | |
| | | | | _{M}^{S}U_{F}-C7OH- | | | | | A_{M}C_{F}C_{M}^{S}T_{D}^{S}T_{D}- | | | |
| | | | | [_{D}Tx-01-08] | | | | | OH-3′ | | | |
| DT- | 494 | 512 | DTS- | 5′-OH- | 669 | UUUACA | 1109 | DTS- | 5′-PO4- | 226 | AAGAUU | 506 |
| 001198 | | | 001616 | U_{F}^{S}U_{M}^{S}U_{F}A_{M}C_{F}A | | UCACUG | | 001338 | A_{M}^{S}A_{F}^{S}G_{M}A_{F}U_{M} | | CCAGUG | |
| | | | | _{M}U_{F}C_{M}A_{F}C_{M}U_{F}G | | GAAUCU | | | U_{F}C_{M}C_{F}A_{M}G_{F}U | | AUGUAA | |
| | | | | _{M}G_{F}A_{M}A_{F}U_{M}C_{F}^{S}U | | U | | | _{M}G_{F}A_{M}U_{F}G_{M}U_{F} | | ATT | |
| | | | | _{M}^{S}U_{F}-C7OH- | | | | | A_{M}A_{F}A_{M}^{S}T_{D}^{S}T_{D}- | | | |
| | | | | [_{D}Tx-01-08] | | | | | OH-3′ | | | |
| DT- | 501 | 519 | DTS- | 5′-OH- | 738 | CACUGG | 1006 | DTS- | 5′-PO4- | 227 | AAUUUG | 515 |
| 001199 | | | 001835 | C_{F}^{S}A_{M}^{S}CHU_{M}G_{F}G | | AAUCUU | | 001340 | A_{M}^{S}A_{F}^{S}U_{M}U_{F}U_{M} | | GAAGAU | |
| | | | | _{M}A_{F}A_{M}U_{F}C_{M}U_{F}U | | CCAAAU | | | G_{F}G_{M}A_{F}A_{M}G_{F}A | | UCCAGU | |
| | | | | _{M}C_{F}C_{M}A_{F}A_{M}A_{F}^{S}U | | U | | | _{M}U_{F}U_{M}C_{F}C_{M}A_{F} | | GTT | |
| | | | | _{M}^{S}U_{F}-C7OH- | | | | | G_{M}U_{F}G_{M}^{S}T_{D}^{S}T_{D}- | | | |
| | | | | [_{D}Tx-01-08] | | | | | OH-3′ | | | |
| DT- | 507 | 525 | DTS- | 5′-OH- | 739 | AAUCUU | 983 | DTS- | 5′-PO4- | 228 | AGCAAG | 533 |
| 001200 | | | 001836 | A_{F}^{S}A_{M}^{S}U_{F}C_{M}U_{F}U | | CCAAAU | | 001342 | A_{M}^{S}G_{F}^{S}C_{M}A_{F}A_{M} | | AAUUUG | |
| | | | | _{M}C_{F}C_{M}A_{F}A_{M}A_{F}U | | UCUUGC | | | G_{F}A_{M}A_{F}U_{M}U_{F}U | | GAAGAU | |
| | | | | _{M}U_{F}C_{M}U_{F}U_{M}G_{F}^{S}C | | U | | | _{M}G_{F}G_{M}A_{F}A_{M}G_{F} | | UTT | |
| | | | | _{M}^{S}U_{F}-C7OH- | | | | | A_{M}U_{F}U_{M}^{S}T_{D}^{S}T_{D}- | | | |
| | | | | [_{D}Tx-01-08] | | | | | OH-3′ | | | |
| DT- | 536 | 554 | DTS- | 5′-OH- | 740 | UGAUGA | 1096 | DTS- | 5′-PO4- | 23 | AUGGCC | 561 |
| 001201 | | | 001837 | U_{F}^{S}G_{M}^{S}A_{F}U_{M}G_{F}A | | GUGCUG | | 001348 | A_{M}^{S}U_{F}^{S}G_{M}G_{F}C_{M} | | GCAGCAC | |
| | | | | _{M}G_{F}U_{M}G_{F}C_{M}U_{F}G | | CGGCCAU | | | C_{F}G_{M}C_{F}A_{M}G_{F}C | | UCAUCAT | |
| | | | | _{M}C_{F}G_{M}G_{F}C_{M}C_{F}^{S}A | | | | | _{M}A_{F}C_{M}U_{F}C_{M}A_{F} | | T | |
| | | | | _{M}^{S}U_{F}-C7OH- | | | | | U_{M}C_{F}A_{M}^{S}T_{D}^{S}T_{D}- | | | |
| | | | | [_{D}Tx-01-08] | | | | | OH-3′ | | | |
| DT- | 569 | 587 | DTS- | 5′-OH- | 741 | ACCCGGA | 987 | DTS- | 5′-PO4- | 236 | UUGAGA | 622 |
| 001202 | | | 001838 | A_{F}^{S}C_{M}^{S}C_{F}C_{M}G_{F}G | | GUGGCA | | 001358 | U_{M}^{S}U_{F}^{S}G_{M}A_{F}G_{M} | | UGCCACU | |
| | | | | _{M}A_{F}G_{M}U_{F}G_{M}G_{F}C | | UCUCAA | | | A_{F}U_{M}G_{F}C_{M}C_{F}A | | CCGGGUT | |
| | | | | _{M}A_{F}U_{M}C_{F}U_{M}C_{F}^{S}A | | | | | _{M}C_{F}U_{M}C_{F}C_{M}G_{F} | | T | |
| | | | | _{M}^{S}A_{F}-C7OH- | | | | | G_{M}G_{F}U_{M}^{S}T_{D}^{S}T_{D}- | | | |
| | | | | [_{D}Tx-01-08] | | | | | OH-3′ | | | |
| DT- | 590 | 608 | DTS- | 5′-OH- | 742 | CGGAUU | 1025 | DTS- | 5′-PO4- | 239 | AAACCG | 493 |
| 001203 | | | 001839 | C_{F}^{S}G_{M}^{S}G_{F}A_{M}U_{F}U | | ACUCCUA | | 001364 | A_{M}^{S}A_{F}^{S}A_{M}C_{F}C_{M} | | UAGGAG | |
| | | | | _{M}A_{F}C_{M}U_{F}C_{M}C_{F}U_{M} | | CGGUUU | | | G_{F}U_{M}A_{F}G_{M}G_{F}A | | UAAUCC | |
| | | | | A_{F}C_{M}G_{F}G_{M}U_{F}^{S}U_{M} | | | | | _{M}G_{F}U_{M}A_{F}A_{M}U_{F} | | GTT | |
| | | | | ^{S}U_{F}-C7OH-[_{D}Tx- | | | | | C_{M}C_{F}G_{M}^{S}T_{D}^{S}T_{D}- | | | |
| | | | | 01-08] | | | | | OH-3′ | | | |
| DT- | 599 | 617 | DTS- | 5′-OH- | 743 | CCUACGG | 1016 | DTS- | 5′-PO4- | 240 | AUGUAG | 563 |
| 001204 | | | 001840 | C_{F}^{S}C_{M}^{S}U_{F}A_{M}C_{F}G | | UUUCGCC | | 001366 | A_{M}^{S}U_{F}^{S}G_{M}U_{F}A_{M} | | GCGAAA | |
| | | | | _{M}G_{F}U_{M}U_{F}U_{M}C_{F}G | | UACAU | | | G_{F}G_{M}C_{F}G_{M}A_{F}A | | CCGUAG | |
| | | | | _{M}C_{F}C_{M}U_{F}A_{M}C_{F}^{S}A | | | | | _{M}A_{F}C_{M}C_{F}G_{M}U_{F} | | GTT | |
| | | | | _{M}^{S}U_{F}-C7OH- | | | | | A_{M}G_{F}G_{M}^{S}T_{D}^{S}T_{D}- | | | |
| | | | | [_{D}Tx-01-08] | | | | | OH-3′ | | | |
| DT- | 602 | 620 | DTS- | 5′-OH- | 744 | ACGGUU | 990 | DTS- | 5′-PO4- | 241 | AGGAUG | 535 |
| 001205 | | | 001841 | A_{F}^{S}C_{M}^{S}G_{F}G_{M}U_{F}U | | UCGCCUA | | 001368 | A_{M}^{S}G_{F}^{S}G_{M}A_{F}U_{M} | | UAGGCG | |
| | | | | _{M}U_{F}C_{M}G_{F}C_{M}C_{F}U_{M} | | CAUCCU | | | G_{F}U_{M}A_{F}G_{M}G_{F}C | | AAACCG | |
| | | | | A_{F}C_{M}A_{F}U_{M}C_{F}^{S}C_{M} | | | | | _{M}G_{F}A_{M}A_{F}A_{M}C_{F} | | UTT | |
| | | | | ^{S}U_{F}-C7OH-[_{D}Tx- | | | | | C_{M}G_{F}U_{M}^{S}T_{D}^{S}T_{D}- | | | |
| | | | | 01-08] | | | | | OH-3′ | | | |
| DT- | 653 | 671 | DTS- | 5′-OH- | 745 | GCGGUG | 1058 | DTS- | 5′-PO4- | 244 | AUCACA | 545 |
| 001206 | | | 001842 | G_{F}^{S}C_{M}^{S}G_{F}G_{M}U_{F}G | | UCAUCU | | 001374 | A_{M}^{S}U_{F}^{S}C_{M}A_{F}C_{M} | | UAGAUG | |
| | | | | _{M}U_{F}C_{M}A_{F}U_{M}C_{F}U | | AUGUGA | | | A_{F}U_{M}A_{F}G_{M}A_{F}U | | ACACCGC | |
| | | | | _{M}A_{F}U_{M}G_{F}U_{M}G_{F}^{S}A | | U | | | _{M}G_{F}A_{M}C_{F}A_{M}C_{F} | | TT | |
| | | | | _{M}^{S}U_{F}-C7OH- | | | | | C_{M}G_{F}C_{M}^{S}T_{D}^{S}T_{D}- | | | |
| | | | | [_{D}Tx-01-08] | | | | | OH-3′ | | | |
| DT- | 656 | 674 | DTS- | 5′-OH- | 746 | GUGUCA | 1076 | DTS- | 5′-PO4- | 245 | AAGAUC | 504 |
| 001207 | | | 001843 | G_{F}^{S}U_{M}^{S}G_{F}U_{M}C_{F}A | | UCUAUG | | 001376 | A_{M}^{S}A_{F}^{S}G_{M}A_{F}U_{M} | | ACAUAG | |
| | | | | _{M}U_{F}C_{M}U_{F}A_{M}U_{F}G | | UGAUCU | | | C_{F}A_{M}C_{F}A_{M}U_{F}A | | AUGACA | |
| | | | | _{M}U_{F}G_{M}A_{F}U_{M}C_{F}^{S}U | | U | | | _{M}G_{F}A_{M}U_{F}G_{M}A_{F} | | CTT | |
| | | | | _{M}^{S}U_{F}-C7OH- | | | | | C_{M}A_{F}C_{M}^{S}T_{D}^{S}T_{D}- | | | |
| | | | | [_{D}Tx-01-08] | | | | | OH-3′ | | | |
| DT- | 663 | 681 | DTS- | 5′-OH- | 747 | CUAUGU | 1032 | DTS- | 5′-PO4- | 246 | UUUCCGC | 631 |
| 001208 | | | 001844 | C_{F}^{S}U_{M}^{S}A_{F}U_{M}G_{F}U | | GAUCUU | | 001378 | U_{M}^{S}U_{F}^{S}U_{M}C_{F}C_{M} | | AAGAUC | |
| | | | | _{M}G_{F}A_{M}U_{F}C_{M}U_{F}U | | GCGGAA | | | G_{F}C_{M}A_{F}A_{M}G_{F}A | | ACAUAGT | |
| | | | | _{M}G_{F}C_{M}G_{F}G_{M}A_{F}^{S}A | | A | | | _{M}U_{F}C_{M}A_{F}C_{M}A_{F} | | T | |
| | | | | _{M}^{S}A_{F}-C7OH- | | | | | U_{M}A_{F}G_{M}^{S}T_{D}^{S}T_{D}- | | | |
| | | | | [_{D}Tx-01-08] | | | | | OH-3′ | | | |
| DT- | 735 | 755 | DTS- | 5′-OH- | 752 | GGGAGG | 1069 | DTS- | 5′-PO4- | 881 | UUUCUG | 1168 |
| 001217 | | | 001857 | G_{F}^{S}G_{M}^{S}G_{F}A_{M}G_{F}G | | AAGGGA | | 001846 | U_{M}^{S}U_{F}^{S}U_{M}C_{F}U_{M} | | UUUUCCC | |
| | | | | _{M}A_{F}A_{M}G_{F}G_{M}G_{F}A | | AAACAG | | | G_{F}U_{M}U_{F}U_{M}U_{F}C | | UUCCUCC | |
| | | | | _{M}A_{F}A_{M}A_{F}C_{M}A_{F}G | | AAA | | | _{M}C_{F}C_{M}U_{F}U_{M}C_{F}C | | CUU | |
| | | | | _{M}A_{F}^{S}A_{M}^{S}A_{F}- | | | | | _{M}U_{F}C_{M}C_{F}C_{M}^{S}U_{M} | | | |
| | | | | C7OH-[_{D}Tx-01- | | | | | ^{S}U_{M}-OH-3′ | | | |
| | | | | 08] | | | | | | | | |
| DT- | 777 | 797 | DTS- | 5′-OH- | 753 | AGCCCAA | 993 | DTS- | 5′-PO4- | 882 | UUGAGU | 1164 |
| 001218 | | | 001858 | A_{F}^{S}G_{M}^{S}C_{F}C_{M}C_{F}A | | AAUCCCA | | 001848 | U_{M}^{S}U_{F}^{S}G_{M}A_{F}G_{M} | | UUGGGA | |
| | | | | _{M}A_{F}A_{M}A_{F}U_{M}C_{F}C | | AACUCA | | | U_{F}U_{M}U_{F}G_{M}G_{F}G | | UUUUGG | |
| | | | | _{M}C_{F}A_{M}A_{F}A_{M}C_{F}U | | A | | | _{M}A_{F}U_{M}U_{F}U_{M}U_{F} | | GCUCG | |
| | | | | _{M}C_{F}A_{M}A_{F}-C7OH- | | | | | G_{M}G_{F}G_{M}C_{F}U_{M}^{S} | | | |
| | | | | [_{D}Tx-01-08] | | | | | C_{M}^{S}G_{M}-OH-3′ | | | |
| DT- | 831 | 851 | DTS- | 5′-OH- | 754 | UUGCUG | 1108 | DTS- | 5′-PO4- | 883 | UACAUC | 1156 |
| 001219 | | | 001859 | U_{F}^{S}U_{M}^{S}G_{F}C_{M}U_{F}G | | UUGAUU | | 001850 | U_{M}^{S}A_{F}^{S}C_{M}A_{F}U_{M} | | UUCAAU | |
| | | | | _{M}U_{F}U_{M}G_{F}A_{M}U_{F}U | | GAAGAU | | | C_{F}U_{M}U_{F}C_{M}A_{F}A | | CAACAGC | |
| | | | | _{M}G_{F}A_{M}A_{F}G_{M}A_{F}U | | GUA | | | _{M}U_{F}C_{M}A_{F}A_{M}C_{F} | | AACC | |
| | | | | _{M}G_{F}^{S}U_{M}^{S}A_{F}- | | | | | A_{M}G_{F}C_{M}A_{F}A_{M}^{S} | | | |
| | | | | C7OH-[_{D}Tx-01- | | | | | C_{M}^{S}C_{M}-OH-3′ | | | |
| | | | | 08] | | | | | | | | |
| DT. | 948 | 968 | DTS- | 5′-OH- | 755 | GAGCCU | 1051 | DTS- | 5′-PO4- | 884 | UUAGCU | 1158 |
| 001220 | | | 001860 | G_{F}^{S}A_{M}^{S}G_{F}C_{M}C_{F}U | | UAAAGA | | 001852 | U_{M}^{S}U_{F}^{S}A_{M}G_{F}C_{M} | | ACUUCU | |
| | | | | _{M}U_{F}A_{M}A_{F}A_{M}G_{F}A | | AGUAGC | | | U_{F}A_{M}C_{F}U_{M}U_{F}C | | UUAAGG | |
| | | | | _{M}A_{F}G_{M}U_{F}A_{M}G_{F}C | | UAA | | | _{M}U_{F}U_{M}U_{F}A_{M}A_{F} | | CUCAA | |
| | | | | _{M}U_{F}^{S}A_{M}^{S}A_{F}- | | | | | G_{M}G_{F}C_{M}U_{F}C_{M}^{S} | | | |
| | | | | C7OH-[_{D}Tx-01- | | | | | A_{M}^{S}A_{M}-OH-3′ | | | |
| | | | | 08] | | | | | | | | |
| DT- | 735 | 755 | DTS- | 5′-OH- | 752 | GGGAGG | 1069 | DTS- | 5′-VP- | 885 | UUUCUG | 1168 |
| 001221 | | | 001857 | G_{F}^{S}G_{M}^{S}G_{F}A_{M}G_{F}G | | AAGGGA | | 001853 | U_{M}^{S}U_{F}^{S}U_{M}C_{F}U_{M} | | UUUUCCC | |
| | | | | _{M}A_{F}A_{M}G_{F}G_{M}G_{F}A | | AAACAG | | | G_{F}U_{M}U_{F}U_{M}U_{F}C | | UUCCUCC | |
| | | | | _{M}A_{F}A_{M}A_{F}C_{M}A_{F}G | | AAA | | | _{M}C_{F}C_{M}U_{F}U_{M}C_{F}C | | CUU | |
| | | | | _{M}A_{F}^{S}A_{M}^{S}A_{F}- | | | | | _{M}U_{F}C_{M}C_{F}C_{M}^{S}U_{M} | | | |
| | | | | C7OH-[_{D}Tx-01- | | | | | ^{S}U_{M}-OH-3′ | | | |
| | | | | 08] | | | | | | | | |
| DT- | 777 | 797 | DTS- | 5′-OH- | 753 | AGCCCAA | 993 | DTS- | 5′-VP- | 886 | UUGAGU | 1164 |
| 001222 | | | 001858 | A_{F}^{S}G_{M}^{S}C_{F}C_{M}C_{F}A | | AAUCCCA | | 001854 | U_{M}^{S}U_{F}^{S}G_{M}A_{F}G_{M} | | UUGGGA | |
| | | | | _{M}A_{F}A_{M}A_{F}U_{M}C_{F}C | | AACUCA | | | U_{F}U_{M}U_{F}G_{M}G_{F}G | | UUUUGG | |
| | | | | _{M}C_{F}A_{M}A_{F}A_{M}C_{F}U | | A | | | _{M}A_{F}U_{M}U_{F}U_{M}U_{F} | | GCUCG | |
| | | | | _{M}C_{F}A_{M}A_{F}-C7OH- | | | | | G_{M}G_{F}G_{M}C_{F}U_{M}^{S} | | | |
| | | | | [_{D}Tx-01-08] | | | | | C_{M}^{S}G_{M}-OH-3′ | | | |
| DT- | 831 | 851 | DTS- | 5′-OH- | 754 | UUGCUG | 1108 | DTS- | 5′-VP- | 887 | UACAUC | 1156 |
| 001223 | | | 001859 | U_{F}^{S}U_{M}^{S}G_{F}C_{M}U_{F}G | | UUGAUU | | 001855 | U_{M}^{S}A_{F}^{S}C_{M}A_{F}U_{M} | | UUCAAU | |
| | | | | _{M}U_{F}U_{M}G_{F}A_{M}U_{F}U | | GAAGAU | | | C_{F}U_{M}U_{F}C_{M}A_{F}A | | CAACAGC | |
| | | | | _{M}G_{F}A_{M}A_{F}G_{M}A_{F}U | | GUA | | | _{M}U_{F}C_{M}A_{F}A_{M}C_{F} | | AACC | |
| | | | | _{M}G_{F}^{S}U_{M}^{S}A_{F}- | | | | | A_{M}G_{F}C_{M}A_{F}A_{M}^{S} | | | |
| | | | | C7OH-[_{D}Tx-01- | | | | | C_{M}^{S}C_{M}-OH-3′ | | | |
| | | | | 08] | | | | | | | | |
| DT- | 948 | 968 | DTS- | 5′-OH- | 755 | GAGCCU | 1051 | DTS- | 5′-VP- | 888 | UUAGCU | 1158 |
| 001224 | | | 001860 | G_{F}^{S}A_{M}^{S}G_{F}C_{M}C_{F}U | | UAAAGA | | 001856 | U_{M}^{S}U_{F}^{S}A_{M}G_{F}C_{M} | | ACUUCU | |
| | | | | _{M}U_{F}A_{M}A_{F}A_{M}G_{F}A | | AGUAGC | | | U_{F}A_{M}C_{F}U_{M}U_{F}C | | UUAAGG | |
| | | | | _{M}A_{F}G_{M}U_{F}A_{M}G_{F}C | | UAA | | | _{M}U_{F}U_{M}U_{F}A_{M}A_{F} | | CUCAA | |
| | | | | _{M}U_{F}^{S}A_{M}^{S}A_{F}- | | | | | G_{M}G_{F}C_{M}U_{F}C_{M}^{S} | | | |
| | | | | C7OH-[_{D}Tx-01- | | | | | A_{M}^{S}A_{M}-OH-3′ | | | |
| | | | | 08] | | | | | | | | |
| DT- | 975 | 993 | DTS- | 5′-OH- | 761 | UUACAU | 1104 | DTS- | 5′-PO4- | 261 | UUAUAC | 642 |
| 001230 | | | 001871 | U_{F}^{S}U_{M}^{S}A_{F}C_{M}A_{F}U | | CCUAACA | | 001408 | U_{M}^{S}U_{F}^{S}A_{M}U_{F}A_{M} | | UGUUAG | |
| | | | | _{M}C_{F}C_{M}U_{F}A_{M}A_{F}C_{M} | | GUAUAA | | | C_{F}U_{M}G_{F}U_{M}U_{F}A | | GAUGUA | |
| | | | | A_{F}G_{M}U_{F}A_{M}U_{F}^{S}A_{M} | | | | | _{M}G_{F}G_{M}A_{F}U_{M}G_{F} | | ATT | |
| | | | | ^{S}A_{F}-C7OH- | | | | | U_{M}A_{F}A_{M}^{S}T_{D}^{S}T_{D}- | | | |
| | | | | [_{D}Tx-01-08] | | | | | OH-3′ | | | |
| DT- | 1039 | 1057 | DTS- | 5′-OH- | 762 | UUACCCA | 1105 | DTS- | 5′-PO4- | 263 | UUAUCU | 643 |
| 001231 | | | 001872 | U_{F}^{S}U_{M}^{S}A_{F}C_{M}C_{F}C | | GAAAUA | | 001412 | U_{M}^{S}U_{F}^{S}A_{M}U_{F}C_{M} | | UAUUUC | |
| | | | | _{M}A_{F}G_{M}A_{F}A_{M}A_{F}U | | AGAUAA | | | U_{F}U_{M}A_{F}U_{M}U_{F}U | | UGGGUA | |
| | | | | _{M}A_{F}A_{M}G_{F}A_{M}U_{F}^{S}A | | | | | _{M}C_{F}U_{M}G_{F}G_{M}G_{F} | | ATT | |
| | | | | _{M}^{S}A_{F}-C7OH- | | | | | U_{M}A_{F}A_{M}^{S}T_{D}^{S}T_{D}- | | | |
| | | | | [_{D}Tx-01-08] | | | | | OH-3′ | | | |
| DT- | 1757 | 1775 | DTS- | 5′-OH- | 763 | UGCUUU | 1099 | DTS- | 5′-PO4- | 269 | AAUCAG | 644 |
| 001232 | | | 001873 | U_{F}^{S}G_{M}^{S}C_{F}U_{M}U_{F}U | | GCAUUU | | 001424 | A_{M}^{S}A_{F}^{S}U_{M}C_{F}A_{M} | | AAAAUG | |
| | | | | _{M}G_{F}C_{M}A_{F}U_{M}U_{F}U | | UCUGAU | | | G_{F}A_{M}A_{F}A_{M}A_{F}U | | CAAAGC | |
| | | | | _{M}U_{F}C_{M}U_{F}G_{M}A_{F}^{S}U | | U | | | _{M}G_{F}C_{M}A_{F}A_{M}A_{F} | | ATT | |
| | | | | _{M}^{S}U_{F}-C7OH- | | | | | G_{M}C_{F}A_{M}^{S}T_{D}^{S}T_{D}- | | | |
| | | | | [_{D}Tx-01-08] | | | | | OH-3′ | | | |
| DT- | 1782 | 1800 | DTS- | 5′-OH- | 764 | CAACUG | 1003 | DTS- | 5′-PO4- | 271 | AUCUUA | |
| 001233 | | | 001874 | C_{F}^{S}A_{M}^{S}A_{F}C_{M}U_{F}G | | UGUGGA | | 001428 | A_{M}^{S}U_{F}^{S}C_{M}U_{F}U_{M} | | GUCCACA | |
| | | | | _{M}U_{F}G_{M}U_{F}G_{M}G_{F}A | | CUAAGA | | | A_{F}G_{M}U_{F}C_{M}C_{F}A | | CAGUUGT | |
| | | | | _{M}C_{F}U_{M}A_{F}A_{M}G_{F}^{S}A | | U | | | _{M}C_{F}A_{M}C_{F}A_{M}G_{F} | | T | |
| | | | | _{M}^{S}U_{F}-C7OH- | | | | | U_{M}U_{F}G_{M}^{S}T_{D}^{S}T_{D}- | | | |
| | | | | [_{D}Tx-01-08] | | | | | OH-3′ | | | 645 |
| DT- | 973 | 993 | DTS- | 5′-OH- | 765 | CUUUAC | 1047 | DTS- | 5′-PO4- | 889 | UUAUAC | 1160 |
| 001234 | | | 001875 | C_{F}^{S}U_{M}^{S}U_{F}U_{M}A_{F}C | | AUCCUA | | 001862 | U_{M}^{S}U_{F}^{S}A_{M}U_{F}A_{M} | | UGUUAG | |
| | | | | _{M}A_{F}U_{M}C_{F}C_{M}U_{F}A | | ACAGUA | | | C_{F}U_{M}G_{F}U_{M}U_{F}A | | GAUGUA | |
| | | | | _{M}A_{F}C_{M}A_{F}G_{M}U_{F}A | | UAA | | | _{M}G_{F}G_{M}A_{F}U_{M}G_{F} | | AAGUU | |
| | | | | _{M}U_{F}^{S}A_{M}^{S}A_{F}- | | | | | U_{M}A_{F}A_{M}A_{F}G_{M} | | | |
| | | | | C7OH-[_{D}Tx-01- | | | | | U_{M}^{S}U_{M}-OH-3′ | | | |
| | | | | 08] | | | | | | | | |
| DT- | 1037 | 1057 | DTS- | 5′-OH- | 766 | UUUUAC | 1111 | DTS- | 5′-PO4- | 890 | UUAUCU | 1161 |
| 001235 | | | 001876 | U_{F}^{S}U_{M}^{S}U_{F}U_{M}A_{F}C | | CCAGAA | | 001864 | U_{M}^{S}U_{F}^{S}A_{M}U_{F}C_{M} | | UAUUUC | |
| | | | | _{M}C_{F}C_{M}A_{F}G_{M}A_{F}A | | AUAAGA | | | U_{F}U_{M}A_{F}U_{M}U_{F}U | | UGGGUA | |
| | | | | _{M}A_{F}U_{M}A_{F}A_{M}G_{F}A | | UAA | | | _{M}C_{F}U_{M}G_{F}G_{M}G_{F} | | AAACA | |
| | | | | _{M}U_{F}^{S}A_{M}^{S}A_{F}- | | | | | U_{M}A_{F}A_{M}A_{F}A_{M}^{S} | | | |
| | | | | C7OH-[_{D}Tx-01- | | | | | C_{M}^{S}A_{M}-OH-3′ | | | |
| | | | | 08] | | | | | | | | |
| DT- | 1693 | 1713 | DTS- | 5′-OH- | 767 | GGCCACC | 1066 | DTS- | 5′-PO4- | 891 | AUACAU | 1136 |
| 001236 | | | 001877 | G_{F}^{S}G_{M}^{S}C_{F}C_{M}A_{F}C | | AACUGU | | 001866 | A_{M}^{S}U_{F}^{S}A_{M}C_{F}A_{M} | | CUACAG | |
| | | | | _{M}C_{F}A_{M}A_{F}C_{M}U_{F}G | | AGAUGU | | | U_{F}C_{M}U_{F}A_{M}C_{F}A | | UUGGUG | |
| | | | | _{M}U_{F}A_{M}G_{F}A_{M}U_{F}G | | AU | | | _{M}G_{F}U_{M}U_{F}G_{M}G_{F} | | GCCAA | |
| | | | | _{M}U_{F}^{S}A_{M}^{S}U_{F}- | | | | | U_{M}G_{F}G_{M}C_{F}C_{M}^{S} | | | |
| | | | | C7OH-[_{D}Tx-01- | | | | | A_{M}^{S}A_{M}-OH-3′ | | | |
| | | | | 08] | | | | | | | | |
| DT- | 1755 | 1775 | DTS- | 5′-OH- | 768 | UUUGCU | 1110 | DTS- | 5′-PO4- | 892 | AAUCAG | 1122 |
| 001237 | | | 001878 | U_{F}^{S}U_{M}^{S}U_{F}G_{M}C_{F}U | | UUGCAU | | 001868 | A_{M}^{S}A_{F}^{S}U_{M}C_{F}A_{M} | | AAAAUG | |
| | | | | _{M}U_{F}U_{M}G_{F}C_{M}A_{F}U | | UUUCUG | | | G_{F}A_{M}A_{F}A_{M}A_{F}U | | CAAAGC | |
| | | | | _{M}U_{F}U_{M}U_{F}C_{M}U_{F}G | | AUU | | | _{M}G_{F}C_{M}A_{F}A_{M}A_{F} | | AAAAA | |
| | | | | _{M}A_{F}^{S}U_{M}^{S}U_{F}- | | | | | G_{M}C_{F}A_{M}A_{F}A_{M}^{S} | | | |
| | | | | C7OH-[_{D}Tx-01- | | | | | A_{M}^{S}A_{M}-OH-3′ | | | |
| | | | | 08] | | | | | | | | |
| DT- | 1780 | 1800 | DTS- | 5′-OH- | 769 | ACCAACU | 986 | DTS- | 5′-PO4- | 893 | AUCUUA | 1142 |
| 001238 | | | 001879 | A_{F}^{S}C_{M}^{S}C_{F}A_{M}A_{F}C | | GUGUGG | | 001870 | A_{M}^{S}U_{F}^{S}C_{M}U_{F}U_{M} | | GUCCACA | |
| | | | | _{M}U_{F}G_{M}U_{F}G_{M}U_{F}G | | ACUAAG | | | A_{F}G_{M}U_{F}C_{M}C_{F}A | | CAGUUG | |
| | | | | _{M}G_{F}A_{M}C_{F}U_{M}A_{F}A | | AU | | | _{M}C_{F}A_{M}C_{F}A_{M}G_{F} | | GUAU | |
| | | | | _{M}G_{F}^{S}A_{M}^{S}U_{F}- | | | | | U_{M}U_{F}G_{M}G_{F}U_{M}^{S} | | | |
| | | | | C7OH-[_{D}Tx-01- | | | | | A_{M}^{S}U_{M}-OH-3′ | | | |
| | | | | 08] | | | | | | | | |
| DT- | 973 | 993 | DTS- | 5′-OH- | 765 | CUUUAC | 1047 | DTS- | 5′-VP- | 894 | UUAUAC | 1160 |
| 001239 | | | 001875 | C_{F}^{S}U_{M}^{S}U_{F}U_{M}A_{F}C | | AUCCUA | | 001880 | U_{M}^{S}U_{F}^{S}A_{M}U_{F}A_{M} | | UGUUAG | |
| | | | | _{M}A_{F}U_{M}C_{F}C_{M}U_{F}A | | ACAGUA | | | C_{F}U_{M}G_{F}U_{M}U_{F}A | | GAUGUA | |
| | | | | _{M}A_{F}C_{M}A_{F}G_{M}U_{F}A | | UAA | | | _{M}G_{F}G_{M}A_{F}U_{M}G_{F} | | AAGUU | |
| | | | | _{M}U_{F}^{S}A_{M}^{S}A_{F}- | | | | | U_{M}A_{F}A_{M}A_{F}G^{S} | | | |
| | | | | C7OH-[_{D}Tx-01- | | | | | U_{M}^{S}U_{M}-OH-3′ | | | |
| | | | | 08] | | | | | | | | |
| DT- | 1037 | 1057 | DTS- | 5′-OH- | 766 | UUUUAC | 1111 | DTS- | 5′-VP- | 895 | UUAUCU | 1161 |
| 001240 | | | 001876 | U_{F}^{S}U_{M}^{S}U_{F}U_{M}A_{F}C | | CCAGAA | | 001881 | U_{M}^{S}U_{F}^{S}A_{M}U_{F}C_{M} | | UAUUUC | |
| | | | | _{M}C_{F}C_{M}A_{F}G_{M}A_{F}A | | AUAAGA | | | U_{F}U_{M}A_{F}U_{M}U_{F}U | | UGGGUA | |
| | | | | _{M}A_{F}U_{M}A_{F}A_{M}G_{F}A | | UAA | | | _{M}C_{F}U_{M}G_{F}G_{M}G_{F} | | AAACA | |
| | | | | _{M}U_{F}^{S}A_{M}^{S}A_{F}- | | | | | U_{M}A_{F}A_{M}A_{F}A_{M}^{S} | | | |
| | | | | C7OH-[_{D}Tx-01- | | | | | C_{M}^{S}A_{M}-OH-3′ | | | |
| | | | | 08] | | | | | | | | |
| DT- | 1693 | 1713 | DTS- | 5′-OH- | 767 | GGCCACC | 1066 | DTS- | 5′-VP- | 896 | AUACAU | 1136 |
| 001241 | | | 001877 | G_{F}^{S}G_{M}^{S}C_{F}C_{M}A_{F}C | | AACUGU | | 001882 | A_{M}^{S}U_{F}^{S}A_{M}C_{F}A_{M} | | CUACAG | |
| | | | | _{M}C_{F}A_{M}A_{F}C_{M}U_{F}G | | AGAUGU | | | U_{F}C_{M}U_{F}A_{M}C_{F}A | | UUGGUG | |
| | | | | _{M}U_{F}A_{M}G_{F}A_{M}U_{F}G | | AU | | | _{M}G_{F}U_{M}U_{F}G_{M}G_{F} | | GCCAA | |
| | | | | _{M}U_{F}^{S}A_{M}^{S}U_{F}- | | | | | U_{M}G_{F}G_{M}C_{F}C_{M}^{S} | | | |
| | | | | C7OH-[_{D}Tx-01- | | | | | A_{M}^{S}A_{M}-OH-3′ | | | |
| | | | | 08] | | | | | | | | |
| DT- | 1755 | 1775 | DTS- | 5′-OH- | 768 | UUUGCU | 1110 | DTS- | 5′-VP- | 897 | AAUCAG | 1122 |
| 001242 | | | 001878 | U_{F}^{S}U_{M}^{S}U_{F}G_{M}C_{F}U | | UUGCAU | | 001883 | A_{M}^{S}A_{F}^{S}U_{M}C_{F}A_{M} | | AAAAUG | |
| | | | | _{M}U_{F}U_{M}G_{F}C_{M}A_{F}U | | UUUCUG | | | G_{F}A_{M}A_{F}A_{M}A_{F}U | | CAAAGC | |
| | | | | _{M}U_{F}U_{M}U_{F}C_{M}U_{F}G | | AUU | | | _{M}G_{F}C_{M}A_{F}A_{M}A_{F} | | AAAAA | |
| | | | | _{M}A_{F}^{S}U_{M}^{S}U_{F}- | | | | | G_{M}C_{F}A_{M}A_{F}A_{M}^{S} | | | |
| | | | | C7OH-[_{D}Tx-01- | | | | | A_{M}^{S}A_{M}-OH-3′ | | | |
| | | | | 08] | | | | | | | | |
| DT- | 1780 | 1800 | DTS- | 5′-OH- | 769 | ACCAACU | 986 | DTS- | 5′-VP- | 898 | AUCUUA | 1142 |
| 001243 | | | 001879 | A_{F}^{S}C_{M}^{S}C_{F}A_{M}A_{F}C | | GUGUGG | | 001884 | A_{M}^{S}U_{F}^{S}C_{M}U_{F}U_{M} | | GUCCACA | |
| | | | | _{M}U_{F}G_{M}U_{F}G_{M}U_{F}G | | ACUAAG | | | A_{F}G_{M}U_{F}C_{M}C_{F}A | | CAGUUG | |
| | | | | _{M}G_{F}A_{M}C_{F}U_{M}A_{F}A | | AU | | | _{M}C_{F}A_{M}C_{F}A_{M}G_{F} | | GUAU | |
| | | | | _{M}G_{F}^{S}A_{M}^{S}U_{F}- | | | | | U_{M}U_{F}G_{M}G_{F}U_{M}^{S} | | | |
| | | | | C7OH-[_{D}Tx-01- | | | | | A_{M}^{S}U_{M}-OH-3′ | | | |
| | | | | 08] | | | | | | | | |
| DT- | 213 | 233 | DTS- | 5′-OH- | 770 | CCUCCUG | 1018 | DTS- | 5′-VP- | 899 | AUGAUA | 1144 |
| 001246 | | | 001887 | C_{F}^{S}C_{M}^{S}U_{F}C_{M}C_{F}U | | UUGCUG | | 001888 | A_{M}^{S}U_{F}^{S}G_{M}A_{F}U_{M} | | CUCAGCA | |
| | | | | _{M}G_{F}U_{M}U_{F}G_{M}C_{F}U_{F} | | AGUAUC | | | A_{F}C_{M}U_{F}C_{M}A_{M}G | | ACAGGA | |
| | | | | G_{F}A_{M}G_{F}U_{M}A_{F}U_{M} | | AU | | | _{M}C_{F}A_{M}A_{F}C_{M}A_{F} | | GGAG | |
| | | | | C_{F}^{S}A_{M}^{S}U_{F}-C7OH- | | | | | G_{M}G_{F}A_{M}G_{F}G_{M} | | | |
| | | | | [_{D}Tx-01-08] | | | | | A_{M}^{S}G_{M}-OH-3′ | | | |
| DT- | 213 | 233 | DTS- | 5′-OH- | 771 | CCUCCUG | 1018 | DTS- | 5′-VP- | 900 | AUGAUA | 1144 |
| 001247 | | | 001889 | C_{F}^{S}C_{M}^{S}U_{F}C_{M}C_{F}U | | UUGCUG | | 001890 | A_{M}^{S}U_{F}^{S}G_{M}A_{F}U_{M} | | CUCAGCA | |
| | | | | _{M}G_{F}U_{M}U_{F}G_{F}C_{F}U_{M} | | AGUAUC | | | A_{F}C_{M}U_{F}C_{M}A_{F}G | | ACAGGA | |
| | | | | G_{F}A_{M}G_{F}U_{M}A_{F}U_{M} | | AU | | | _{M}C_{M}A_{M}A_{F}C_{M}A_{F} | | GGAG | |
| | | | | C_{F}^{S}A_{M}^{S}U_{F}-C7OH- | | | | | G_{M}G_{F}A_{M}G_{F}G^{S} | | | |
| | | | | [_{D}Tx-01-08] | | | | | A_{M}^{S}G_{M}-OH-3′ | | | |
| DT- | 213 | 233 | DTS- | 5′-OH- | 772 | CCUCCUG | 1018 | DTS- | 5′-VP- | 879 | AUGAUA | 1144 |
| 001250 | | | 001893 | C_{M}^{S}C_{M}^{S}U_{M}C_{M}C_{F}U | | UUGCUG | | 001218 | A_{M}^{S}U_{F}^{S}G_{M}A_{F}U_{M} | | CUCAGCA | |
| | | | | _{M}G_{F}U_{M}U_{F}G_{M}C_{F}U | | AGUAUC | | | A_{F}C_{M}U_{F}C_{M}A_{F}G | | ACAGGA | |
| | | | | _{M}G_{F}A_{M}G_{F}U_{M}A_{F}U | | AU | | | _{M}C_{F}A_{M}A_{F}C_{M}A_{F} | | GGAG | |
| | | | | _{M}C_{M}^{S}A_{M}^{S}U_{M}- | | | | | G_{M}G_{F}A_{M}G_{F}G_{M}^{S} | | | |
| | | | | C7OH-[_{D}Tx-01- | | | | | A_{M}^{S}G_{M}-OH-3′ | | | |
| | | | | 08] | | | | | | | | |
| DT- | 213 | 233 | DTS- | 5′-OH- | 773 | CCUCCUG | 1018 | DTS- | 5′-VP- | 901 | AUGAUA | 1144 |
| 001251 | | | 001894 | C_{M}^{S}C_{M}^{S}U_{M}C_{M}C_{M} | | UUGCUG | | 001895 | A_{M}^{S}U_{F}^{S}G_{M}A_{F}U_{M} | | CUCAGCA | |
| | | | | U_{M}G_{F}U_{M}U_{F}G_{M}C_{F} | | AGUAUC | | | A_{F}C_{M}U_{M}C_{M}A_{F}G | | ACAGGA | |
| | | | | U_{M}G_{F}A_{M}G_{F}U_{M}A_{F} | | AU | | | _{M}C_{M}A_{M}A_{F}C_{M}A_{F} | | GGAG | |
| | | | | U_{M}C_{M}^{S}A_{M}^{S}U_{M}- | | | | | G_{M}G_{F}A_{M}G_{F}G_{M} | | | |
| | | | | C7OH-[_{D}Tx-01- | | | | | A_{M}^{S}G_{M}-OH-3′ | | | |
| | | | | 08] | | | | | | | | |
| DT- | 213 | 233 | DTS- | 5′-OH- | 774 | CCUCCUG | 1018 | DTS- | 5′-VP- | 902 | AUGAUA | 1144 |
| 001252 | | | 001896 | C_{M}^{S}C_{M}^{S}U_{M}C_{M}C_{M} | | UUGCUG | | 001897 | A_{M}^{S}U_{F}^{S}G_{M}A_{M}U | | CUCAGCA | |
| | | | | U_{M}G_{F}U_{M}U_{F}G_{F}C_{F} | | AGUAUC | | | _{M}A_{F}C_{M}U_{M}C_{M}A_{M} | | ACAGGA | |
| | | | | U_{M}G_{M}A_{M}G_{M}U_{M}A | | AU | | | G_{M}C_{M}A_{M}A_{F}C_{M}A | | GGAG | |
| | | | | _{M}U_{M}C_{M}^{S}A_{M}^{S}U_{M}- | | | | | G_{M}G_{M}A_{M}G_{M}G_{M} | | | |
| | | | | C7OH-[_{D}Tx-01- | | | | | ^{S}A_{M}^{S}G_{M}-OH-3′ | | | |
| | | | | 08] | | | | | | | | |
| DT- | 213 | 233 | DTS- | 5′-OH- | 775 | CCUCCUG | 1018 | DTS- | 5′-VP- | 902 | AUGAUA | 1144 |
| 001253 | | | 001898 | C_{M}^{S}C_{M}^{S}U_{M}C_{M}C_{M} | | UUGCUG | | 001897 | A_{M}^{S}U_{F}^{S}G_{M}A_{M}U | | CUCAGCA | |
| | | | | U_{M}G_{F}U_{M}U_{F}G_{F}C_{F} | | AGUAUC | | | _{M}A_{F}C_{M}U_{M}C_{M}A_{M} | | ACAGGA | |
| | | | | U_{M}G_{M}A_{M}G_{M}U_{M}A | | AU | | | G_{M}C_{M}A_{M}A_{F}C_{M}A | | GGAG | |
| | | | | _{M}U_{M}C_{M}A_{M}U_{M}- | | | | | G_{M}G_{M}A_{M}G_{M}G_{M} | | | |
| | | | | C7OH-[_{D}Tx-01- | | | | | ^{S}A_{M}^{S}G_{M}-OH-3′ | | | |
| | | | | 08] | | | | | | | | |
| DT- | 213 | 233 | DTS- | 5′-OH- | 776 | CCUCCUG | 1018 | DTS- | 5′-VP- | 879 | AUGAUA | 1144 |
| 001254 | | | 001899 | C_{E}^{S}C_{E}^{S}U_{M}C_{M}C_{F}U | | UUGCUG | | 001218 | A_{M}^{S}U_{F}^{S}G_{M}A_{F}U_{M} | | CUCAGCA | |
| | | | | _{M}G_{F}U_{M}U_{F}G_{M}C_{F}U | | AGUAUC | | | A_{F}C_{M}U_{F}C_{M}A_{F}G | | ACAGGA | |
| | | | | _{M}G_{F}A_{M}G_{F}U_{M}A_{F}U | | AU | | | _{M}C_{F}A_{M}A_{F}C_{M}A_{F} | | GGAG | |
| | | | | _{M}C_{M}^{S}A_{M}^{S}U_{M}- | | | | | G_{M}G_{F}A_{M}G_{F}G_{M}^{S} | | | |
| | | | | C7OH-[_{D}Tx-01- | | | | | A_{M}^{S}G_{M}-OH-3′ | | | |
| | | | | 08] | | | | | | | | |
| DT- | 213 | 233 | DTS- | 5′-OH- | 777 | CCTCCUG | 1015 | DTS- | 5′-VP- | 879 | AUGAUA | 1144 |
| 001255 | | | 001900 | C_{M}^{S}C_{E}^{S}U_{E}C_{M}C_{F}U | | UUGCUG | | 001218 | A_{M}^{S}U_{F}^{S}G_{M}A_{F}U_{M} | | CUCAGCA | |
| | | | | _{M}G_{F}U_{M}U_{F}G_{M}C_{F}U | | AGUAUC | | | A_{F}C_{M}U_{F}C_{M}A_{F}G | | ACAGGA | |
| | | | | _{M}G_{F}A_{M}G_{F}U_{M}A_{F}U | | AU | | | _{M}C_{F}A_{M}A_{F}C_{M}AR | | GGAG | |
| | | | | _{M}C_{M}^{S}A_{M}^{S}U_{M}- | | | | | G_{M}G_{F}A_{M}G_{F}G_{M} | | | |
| | | | | C7OH-[_{D}Tx-01- | | | | | A_{M}^{S}G_{M}-OH-3′ | | | |
| | | | | 08] | | | | | | | | |
| DT- | 213 | 233 | DTS- | 5′-OH- | 778 | CCTCCUG | 1015 | DTS- | 5′-VP- | 879 | AUGAUA | 1144 |
| 001256 | | | 001901 | C_{M}^{S}C_{E}^{S}U_{E}C_{M}C_{F}U | | UUGCUG | | 001218 | A_{M}^{S}U_{F}^{S}G_{M}A_{F}U_{M} | | CUCAGCA | |
| | | | | _{M}G_{F}U_{M}U_{F}G_{M}C_{F}U | | AGUAUC | | | A_{F}C_{M}U_{F}C_{M}A_{F}G | | ACAGGA | |
| | | | | _{M}G_{F}A_{M}G_{F}U_{M}A_{F}U | | AU | | | _{M}C_{F}A_{M}A_{F}C_{M}A_{F} | | GGAG | |
| | | | | _{M}C_{M}^{S}A_{M}^{S}U_{M}- | | | | | G_{M}G_{F}A_{M}G_{F}G_{M}^{S} | | | |
| | | | | C7OH-[_{D}Tx-01- | | | | | A_{M}^{S}G_{M}-OH-3′ | | | |
| | | | | 08] | | | | | | | | |
| DT- | 213 | 233 | DTS- | 5′-OH- | 779 | CCTCCUG | 1015 | DTS- | 5′-VP- | 879 | AUGAUA | 1144 |
| 001257 | | | 001902 | C_{E}^{S}C_{E}^{S}U_{E}C_{E}C_{F}U_{M} | | UUGCUG | | 001218 | A_{M}^{S}U_{F}^{S}G_{M}A_{F}U_{M} | | CUCAGCA | |
| | | | | G_{F}U_{M}U_{F}G_{M}C_{F}U_{M} | | AGUAUC | | | A_{F}C_{M}U_{F}C_{M}A_{F}G | | ACAGGA | |
| | | | | G_{F}A_{M}G_{F}U_{M}A_{F}U_{M} | | AU | | | _{M}C_{F}A_{M}A_{F}C_{M}A_{F} | | GGAG | |
| | | | | C_{M}^{S}A_{M}^{S}U_{M}- | | | | | G_{M}G_{F}A_{M}G_{F}G_{M}^{S} | | | |
| | | | | C7OH-[_{D}Tx-01- | | | | | A_{M}^{S}G_{M}-OH-3′ | | | |
| | | | | 08] | | | | | | | | |
| DT- | 215 | 235 | DTS- | 5′-OH- | 780 | UCCUGU | 1091 | DTS- | 5′-VP- | 903 | CGAUGA | 1151 |
| 001261 | | | 001906 | U_{F}^{S}C_{M}^{S}C_{F}U_{M}G_{F}U | | UGCUGA | | 001907 | C_{M}^{S}G_{F}^{S}A_{M}U_{F}G_{M} | | UACUCA | |
| | | | | _{M}U_{F}G_{M}C_{F}U_{M}G_{F}A | | GUAUCA | | | A_{F}U_{M}A_{F}C_{M}U_{F}C | | GCAACA | |
| | | | | _{M}G_{F}U_{M}A_{F}U_{M}C_{F}A | | UCG | | | _{M}A_{F}G_{M}C_{F}A_{M}A_{F} | | GGAGG | |
| | | | | _{M}U_{F}^{S}C_{M}^{S}G_{F}- | | | | | C_{M}A_{F}G_{M}G_{F}A_{M}^{S} | | | |
| | | | | C7OH-[_{D}Tx-01- | | | | | G_{M}^{S}G_{M}-OH-3′ | | | |
| | | | | 08] | | | | | | | | |
| DT- | 217 | 237 | DTS- | 5′-OH- | 781 | CUGUUG | 1045 | DTS- | 5′-VP- | 904 | GACGAU | 1152 |
| 001262 | | | 001908 | C_{F}^{S}U_{M}^{S}G_{F}U_{M}U_{F}G | | CUGAGU | | 001909 | G_{M}^{S}A_{F}^{S}C_{M}G_{F}A_{M} | | GAUACU | |
| | | | | _{M}C_{F}U_{M}G_{F}A_{M}G_{F}U | | AUCAUC | | | U_{F}G_{M}A_{F}U_{M}A_{F}C | | CAGCAAC | |
| | | | | _{M}A_{F}U_{M}C_{F}A_{M}U_{F}C | | GUC | | | _{M}U_{F}C_{M}A_{F}G_{M}C_{F} | | AGGA | |
| | | | | _{M}G_{F}^{S}U_{M}^{S}C_{F}- | | | | | A_{M}A_{F}C_{M}A_{F}G_{M}^{S} | | | |
| | | | | C7OH-[_{D}Tx-01- | | | | | G_{M}^{S}A_{M}-OH-3′ | | | |
| | | | | 08] | | | | | | | | |
| DT- | 218 | 238 | DTS- | 5′-OH- | 782 | UGUUGC | 1103 | DTS- | 5′-VP- | 905 | GGACGA | 1155 |
| 001263 | | | 001910 | U_{F}^{S}G_{M}^{S}U_{F}U_{M}^{S}G_{F} | | UGAGUA | | 001911 | G_{M}^{S}G_{F}^{S}A_{M}C_{F}G_{M} | | UGAUAC | |
| | | | | C_{M}U_{F}G_{M}A_{F}G_{M}U_{F} | | UCAUCG | | | A_{F}U_{M}G_{F}A_{M}U_{F}A | | UCAGCA | |
| | | | | A_{M}U_{F}C_{M}A_{F}U_{M}C_{F} | | UCC | | | _{M}C_{F}U_{M}C_{F}A_{M}G_{F} | | ACAGG | |
| | | | | G_{M}U_{F}^{S}C_{M}^{S}C_{F}- | | | | | C_{M}A_{F}A_{M}C_{F}A_{M}^{S} | | | |
| | | | | C7OH-[_{D}Tx-01- | | | | | G_{M}^{S}G_{M}-OH-3′ | | | |
| | | | | 08] | | | | | | | | |
| DT- | 298 | 318 | DTS- | 5′-OH- | 783 | GGCAAU | 1065 | DTS- | 5′-VP- | 906 | AUCAGU | 1140 |
| 001264 | | | 001912 | G_{F}^{S}G_{M}^{S}C_{F}A_{M}A_{F}U | | GGACAC | | 001913 | A_{M}^{S}U_{F}^{S}C_{M}A_{F}G_{M} | | UGCGUG | |
| | | | | _{M}G_{F}G_{M}A_{F}C_{M}A_{F}C | | GCAACU | | | U_{F}U_{M}G_{F}C_{M}G_{F}U | | UCCAUU | |
| | | | | _{M}G_{F}C_{M}A_{F}A_{M}C_{F}U | | GAU | | | _{M}G_{F}U_{M}C_{F}C_{M}A_{F} | | GCCCA | |
| | | | | _{M}G_{F}^{S}A_{M}^{S}U_{F}- | | | | | U_{M}U_{F}G_{M}C_{F}C_{M}^{S} | | | |
| | | | | C7OH-[_{D}Tx-01- | | | | | C_{M}^{S}A_{M}-OH-3′ | | | |
| | | | | 08] | | | | | | | | |
| DT- | 405 | 425 | DTS- | 5′-OH- | 784 | GGCCACC | 1067 | DTS- | 5′-VP- | 907 | AUCGAC | 1141 |
| 001265 | | | 001914 | G_{F}^{S}G_{M}^{S}C_{F}C_{M}^{S}A_{F}C | | AUGAUC | | 001915 | A_{M}^{S}U_{F}^{S}C_{M}G_{F}A_{M} | | AGGAUC | |
| | | | | _{M}C_{F}A_{M}U_{F}G_{M}A_{F}U | | CUGUCG | | | C_{F}A_{M}G_{F}G_{M}A_{F}U | | AUGGUG | |
| | | | | _{M}C_{F}C_{M}U_{F}G_{M}U_{F}C_{M} | | AU | | | _{M}C_{F}A_{M}U_{F}G_{M}G_{F} | | GCCUG | |
| | | | | G_{F}^{S}A_{M}^{S}U_{F}-C7OH- | | | | | U_{M}G_{F}G_{M}C_{F}C_{M}^{S} | | | |
| | | | | [_{D}Tx-01-08] | | | | | U_{M}^{S}G_{M}-OH-3′ | | | |
| DT- | 417 | 437 | DTS- | 5′-OH- | 785 | CCUGUCG | 1021 | DTS- | 5′-VP- | 908 | AUGCUG | 1147 |
| 001266 | | | 001916 | C_{F}^{S}C_{M}^{S}U_{F}G_{M}U_{F}C | | AUCAUC | | 001917 | A_{M}^{S}U_{F}^{S}G_{M}C_{F}U_{M} | | AAGAUG | |
| | | | | _{M}G_{F}A_{M}U_{F}C_{M}A_{F}U | | UUCAGC | | | G_{F}A_{M}A_{F}G_{M}A_{F}U | | AUCGAC | |
| | | | | _{M}C_{F}U_{M}U_{F}C_{M}A_{F}G | | AU | | | _{M}G_{F}A_{M}U_{F}C_{M}G_{F} | | AGGAU | |
| | | | | _{M}C_{F}^{S}A_{M}^{S}U_{F}- | | | | | A_{M}C_{F}A_{M}G_{F}G_{M}^{S} | | | |
| | | | | C7OH-[_{D}Tx-01- | | | | | A_{M}^{S}U_{M}-OH-3′ | | | |
| | | | | 08] | | | | | | | | |
| DT- | 198 | 218 | DTS- | 5′-OH- | 786 | CCUCCUG | 1019 | DTS- | 5′-VP- | 909 | AUGAUA | 1143 |
| 001267 | | | 001918 | C_{F}^{S}C_{M}^{S}U_{F}C_{M}C_{F}U | | UUGCUG | | 001919 | A_{M}^{S}U_{F}^{S}G_{M}A_{F}U_{M} | | CCCAGCA | |
| | | | | _{M}G_{F}U_{M}U_{F}G_{M}C_{F}U | | GGUAUC | | | A_{F}C_{M}C_{F}C_{M}A_{F}G | | ACAGGA | |
| | | | | _{M}G_{F}G_{M}G_{F}U_{M}A_{F}U | | AU | | | _{M}C_{F}A_{M}A_{F}C_{M}A_{F} | | GGAG | |
| | | | | C7OH-[_{D}Tx-01- | | | | | G_{M}G_{F}A_{M}G_{F}G_{M}^{S} | | | |
| | | | | 08] | | | | | A_{M}^{S}G_{M}-OH-3′ | | | |
| DT- | 208 | 218 | DTS- | 5′-OH- | 795 | AUGCUCC | 1000 | DTS- | 5′-PO4- | 910 | ACUCAGC | 1127 |
| 001276 | | | 001936 | A_{F}^{S}U_{M}^{S}G_{F}C_{M}U_{F}C | | UCCUGU | | 001921 | A_{M}^{S}C_{F}^{S}U_{M}C_{F}A_{M} | | AACAGG | |
| | | | | _{M}C_{F}U_{M}C_{F}C_{M}U_{F}G_{M} | | UGCUGA | | | G_{F}C_{M}A_{F}A_{M}C_{F}A | | AGGAGC | |
| | | | | U_{F}U_{M}G_{F}C_{M}U_{F}G_{M} | | GU | | | _{M}G_{F}G_{M}A_{F}G_{M}G_{F} | | AUUC | |
| | | | | A_{F}^{S}G_{M}^{S}U_{F}-C7OH- | | | | | A_{M}G_{F}C_{M}A_{F}U_{M}^{S} | | | |
| | | | | [_{D}Tx-01-08] | | | | | U_{M}^{S}C_{M}-OH-3′ | | | |
| DT- | 210 | 230 | DTS- | 5′-OH- | 796 | GCUCCUC | 1060 | DTS- | 5′-PO4- | 911 | AUACUC | 1138 |
| 001277 | | | 001937 | G_{F}^{S}C_{M}^{S}U_{F}C_{M}C_{F}U | | CUGUUG | | 001923 | A_{M}^{S}U_{F}^{S}A_{M}C_{F}U_{M} | | AGCAAC | |
| | | | | _{M}C_{F}C_{M}U_{F}G_{M}U_{F}U | | CUGAGU | | | C_{F}A_{M}G_{F}C_{M}A_{F}A | | AGGAGG | |
| | | | | _{M}G_{F}C_{M}U_{F}G_{M}A_{F}G | | AU | | | _{M}C_{F}A_{M}G_{F}G_{M}A_{F} | | AGCAU | |
| | | | | _{M}U_{F}^{S}A_{M}^{S}U_{F}- | | | | | G_{M}G_{F}A_{M}G_{F}C_{M} | | | |
| | | | | C7OH-[_{D}Tx-01- | | | | | A_{M}^{S}U_{M}-OH-3′ | | | |
| | | | | 08] | | | | | | | | |
| DT- | 211 | 231 | DTS- | 5′-OH- | 797 | CUCCUCC | 1034 | DTS- | 5′-PO4- | 912 | GAUACU | 1153 |
| 001278 | | | 001938 | C_{F}^{S}U_{M}^{S}C_{F}C_{M}U_{F}C | | UGUUGC | | 001925 | G_{M}^{S}A_{F}^{S}U_{M}A_{F}C_{M} | | CAGCAAC | |
| | | | | _{M}C_{F}U_{M}G_{F}U_{M}U_{F}G | | UGAGUA | | | U_{F}C_{M}A_{F}G_{M}C_{F}A | | AGGAGG | |
| | | | | _{M}C_{F}U_{M}G_{F}A_{M}G_{F}U | | UC | | | _{M}A_{F}C_{M}A_{F}G_{M}G_{F} | | AGCA | |
| | | | | _{M}A_{F}^{S}U_{M}^{S}C_{F}- | | | | | A_{M}G_{F}G_{M}A_{F}G_{M} | | | |
| | | | | C7OH-[_{D}Tx-01- | | | | | C_{M}^{S}A_{M}-OH-3′ | | | |
| | | | | 08] | | | | | | | | |
| DT- | 212 | 232 | DTS- | 5′-OH- | 798 | UCCUCCU | 1088 | DTS- | 5′-PO4- | 913 | UGAUAC | 1157 |
| 001279 | | | 001939 | U_{F}^{S}C_{M}^{S}C_{F}U_{M}C_{F}C | | GUUGCU | | 001927 | U_{M}^{S}G_{F}^{S}A_{M}U_{F}A_{M} | | UCAGCA | |
| | | | | _{M}U_{F}G_{M}U_{F}U_{M}G_{F}C | | GAGUAU | | | C_{F}U_{M}C_{F}A_{M}G_{F}C | | ACAGGA | |
| | | | | _{M}U_{F}G_{M}A_{F}G_{M}U_{F}A | | CA | | | _{M}A_{F}A_{M}C_{F}A_{M}G_{F} | | GGAGC | |
| | | | | _{M}U_{F}^{S}C_{M}^{S}A_{F}- | | | | | G_{M}A_{F}G_{M}G_{F}A_{M} | | | |
| | | | | C7OH-[_{D}Tx-01- | | | | | G_{M}^{S}C_{M}-OH-3′ | | | |
| | | | | 08] | | | | | | | | |
| DT- | 214 | 234 | DTS- | 5′-OH- | 799 | CUCCUGU | 1037 | DTS- | 5′-PO4- | 914 | GAUGAU | 1154 |
| 001280 | | | 001940 | C_{F}^{S}U_{M}^{S}C_{F}C_{M}U_{F}G | | UGCUGA | | 001929 | G_{M}^{S}A_{F}^{S}U_{M}G_{F}A_{M} | | ACUCAGC | |
| | | | | _{M}U_{F}U_{M}G_{F}C_{M}U_{F}G | | GUAUCA | | | U_{F}A_{M}C_{F}U_{M}C_{F}A | | AACAGG | |
| | | | | _{M}A_{F}G_{M}U_{F}A_{M}U_{F}C | | UC | | | _{M}G_{F}C_{M}A_{F}A_{M}C_{F} | | AGGA | |
| | | | | _{M}A_{F}^{S}U_{M}^{S}C_{F}- | | | | | A_{M}G_{F}G_{M}A_{F}G_{M}^{S} | | | |
| | | | | C7OH-[_{D}Tx-01- | | | | | G_{M}^{S}A_{M}-OH-3′ | | | |
| | | | | 08] | | | | | | | | |
| DT- | 215 | 235 | DTS- | 5′-OH- | 780 | UCCUGU | 1091 | DTS- | 5′-PO4- | 915 | CGAUGA | 1151 |
| 001281 | | | 001906 | U_{F}^{S}C_{M}^{S}C_{F}U_{M}G_{F}U | | UGCUGA | | 001931 | C_{M}^{S}G_{F}^{S}A_{M}U_{F}G_{M} | | UACUCA | |
| | | | | _{M}U_{F}G_{M}C_{F}U_{M}G_{F}A | | GUAUCA | | | A_{F}U_{M}A_{F}C_{M}U_{F}C | | GCAACA | |
| | | | | _{M}G_{F}U_{M}A_{F}U_{M}C_{F}A | | UCG | | | _{M}A_{F}G_{M}C_{F}A_{M}A_{F} | | GGAGG | |
| | | | | _{M}U_{F}^{S}C_{M}^{S}G_{F}- | | | | | C_{M}A_{F}G_{M}G_{F}A_{M}^{S} | | | |
| | | | | C7OH-[_{D}Tx-01- | | | | | G_{M}^{S}G_{M}-OH-3′ | | | |
| | | | | 08] | | | | | | | | |
| DT- | 217 | 237 | DTS- | 5′-OH- | 781 | CUGUUG | 1045 | DTS- | 5′-PO4- | 916 | GACGAU | 1152 |
| 001282 | | | 001908 | C_{F}^{S}U_{M}^{S}G_{F}U_{M}U_{F}G | | CUGAGU | | 001933 | G_{M}^{S}A_{F}^{S}C_{M}G_{F}A_{M} | | GAUACU | |
| | | | | _{M}C_{F}U_{M}G_{F}A_{M}G_{F}U | | AUCAUC | | | U_{F}G_{M}A_{F}U_{M}A_{F}C | | CAGCAAC | |
| | | | | _{M}A_{F}U_{M}C_{F}A_{M}U_{F}C | | GUC | | | _{M}U_{F}C_{M}A_{F}G_{M}C_{F} | | AGGA | |
| | | | | _{M}G_{F}^{S}U_{M}^{S}C_{F}- | | | | | A_{M}A_{F}C_{M}A_{F}G_{M}^{S} | | | |
| | | | | C7OH-[_{D}Tx-01- | | | | | G_{M} A_{M}-OH-3′ | | | |
| | | | | 08] | | | | | | | | |
| DT- | 218 | 238 | DTS- | 5′-OH- | 782 | UGUUGC | 1103 | DTS- | 5′-PO4- | 917 | GGACGA | 1155 |
| 001283 | | | 001910 | U_{F}^{S}G_{M}^{S}U_{F}U_{M}^{S}G_{F} | | UGAGUA | | 001935 | G_{M}^{S}G_{F}^{S}A_{M}C_{F}G_{M} | | UGAUAC | |
| | | | | C_{M}U_{F}G_{M}A_{F}G_{M}U_{F} | | UCAUCG | | | A_{F}U_{M}G_{F}A_{M}U_{F}A | | UCAGCA | |
| | | | | A_{M}U_{F}C_{M}A_{F}U_{M}C_{E} | | UCC | | | _{M}C_{F}U_{M}C_{F}A_{M}G_{F} | | ACAGG | |
| | | | | G_{M}U_{F}^{S}C_{M}^{S}C_{F}- | | | | | C_{M}A_{F}A_{M}C_{F}A_{M}^{S} | | | |
| | | | | C7OH-[_{D}Tx-01- | | | | | G_{M}^{S}G_{M}-OH-3′ | | | |
| | | | | 08] | | | | | | | | |
| DT- | 225 | 245 | DTS- | 5′-OH- | 812 | GAGUAU | 1054 | DTS- | 5′-PO4- | 918 | ACGUGG | 1126 |
| 001296 | | | 001965 | G_{F}^{S}A_{M}^{S}G_{F}U_{M}A_{F}U | | CAUCGUC | | 001942 | A_{M}^{S}C_{F}^{S}G_{M}U_{F}G_{M} | | AGGACG | |
| | | | | _{M}C_{F}A_{M}U_{F}C_{M}G_{F}U | | CUCCACG | | | G_{F}A_{M}G_{F}G_{M}A_{F}C | | AUGAUA | |
| | | | | _{M}C_{F}C_{M}U_{F}C_{M}C_{F}A_{M} | | U | | | _{M}G_{F}A_{M}U_{F}G_{M}A_{F} | | CUCAG | |
| | | | | C_{F}^{S}G_{M}^{S}U_{F}-C7OH- | | | | | U_{M}A_{F}C_{M}U_{F}C_{M}^{S} | | | |
| | | | | [_{D}Tx-01-08] | | | | | A_{M}^{S}G_{M}-OH-3′ | | | |
| DT- | 243 | 263 | DTS- | 5′-OH- | 813 | CGUCGCG | 1028 | DTS- | 5′-PO4- | 919 | AGCAGC | 1131 |
| 001297 | | | 001966 | C_{F}^{S}G_{M}^{S}U_{F}C_{M}G_{F}C | | GUGCUG | | 001944 | A_{M}^{S}G_{F}^{S}C_{M}A_{F}G_{M} | | ACCAGCA | |
| | | | | _{M}G_{F}G_{M}U_{F}G_{M}C_{F}U | | GUGCUG | | | C_{F}A_{M}C_{F}C_{M}A_{F}G | | CCGCGAC | |
| | | | | _{M}G_{F}G_{M}U_{F}G_{M}C_{F}U | | CU | | | _{M}C_{F}A_{M}C_{F}C_{M}G_{F}C | | GUG | |
| | | | | _{M}G_{F}^{S}C_{M}^{S}U_{F}- | | | | | _{M}G_{F}A_{M}C_{F}G_{M}^{S}U | | | |
| | | | | C7OH-[_{D}Tx-01- | | | | | _{M}^{S}G_{M}-OH-3′ | | | |
| | | | | 08] | | | | | | | | |
| DT- | 251 | 271 | DTS- | 5′-OH- | 814 | UGCUGG | 1097 | DTS- | 5′-PO4- | 920 | AGACGA | 1128 |
| 001298 | | | 001967 | U_{F}^{S}G_{M}^{S}C_{F}U_{M}G_{F}G | | UGCUGC | | 001946 | A_{M}^{S}G_{F}^{S}A_{M}C_{F}G_{M} | | ACAGCA | |
| | | | | _{M}U_{F}G_{M}C_{F}U_{M}G_{F}C | | UGUUCG | | | A_{F}A_{M}C_{F}A_{M}G_{F}C | | GCACCAG | |
| | | | | _{M}U_{F}G_{M}U_{F}U_{M}C_{F}G | | UCU | | | _{M}A_{F}G_{M}C_{F}A_{M}C_{F} | | CACC | |
| | | | | _{M}U_{F}^{S}C_{M}^{S}U_{F}- | | | | | C_{M}A_{F}G_{M}C_{F}A_{M}^{S} | | | |
| | | | | C7OH-[_{D}Tx-01- | | | | | C_{M}^{S}C_{M}-OH-3′ | | | |
| | | | | 08] | | | | | | | | |
| DT- | 298 | 318 | DTS- | 5′-OH- | 783 | GGCAAU | 1065 | DTS- | 5′-PO4- | 921 | AUCAGU | 1140 |
| 001299 | | | 001912 | G_{F}^{S}G_{M}^{S}C_{F}A_{M}A_{F}U | | GGACAC | | 001948 | A_{M}^{S}U_{F}^{S}C_{M}A_{F}G_{M} | | UGCGUG | |
| | | | | _{M}G_{F}G_{M}A_{F}C_{M}A_{F}C | | GCAACU | | | U_{F}U_{M}G_{F}C_{M}G_{F}U | | UCCAUU | |
| | | | | _{M}G_{F}C_{M}A_{F}A_{M}C_{F}U | | GAU | | | _{M}G_{F}U_{M}C_{F}C_{M}A_{F} | | GCCCA | |
| | | | | _{M}G_{F}^{S}A_{M}^{S}U_{F}- | | | | | U_{M}U_{F}G_{M}C_{F}C_{M}^{S} | | | |
| | | | | C7OH-[_{D}Tx-01- | | | | | C_{M}^{S}A_{M}-OH-3′ | | | |
| | | | | 08] | | | | | | | | |
| DT- | 302 | 322 | DTS- | 5′-OH- | 815 | AUGGAC | 1001 | DTS- | 5′-PO4- | 922 | AGAGAU | 1129 |
| 001300 | | | 001968 | A_{F}^{S}U_{M}^{S}G_{F}G_{M}A_{F}C | | ACGCAAC | | 001950 | A_{M}^{S}G_{F}^{S}A_{M}G_{F}A_{M} | | CAGUUG | |
| | | | | _{M}A_{F}C_{M}G_{F}C_{M}A_{F}A | | UGAUCU | | | U_{F}C_{M}A_{F}G_{M}U_{F}U | | CGUGUCC | |
| | | | | _{M}C_{F}U_{M}G_{F}A_{M}U_{F}C | | CU | | | _{M}G_{F}C_{M}G_{F}U_{M}G_{F} | | AUUC | |
| | | | | _{M}U_{F}^{S}C_{M}^{S}U_{F}- | | | | | U_{M}C_{F}C_{M}A_{F}U_{M}^{S} | | | |
| | | | | C7OH-[_{D}Tx-01- | | | | | U_{M}^{S}C_{M}-OH-3′ | | | |
| | | | | 08] | | | | | | | | |
| DT- | 348 | 368 | DTS- | 5′-OH- | 816 | AGGAAA | 994 | DTS- | 5′-PO4- | 923 | AAACAG | 1112 |
| 001301 | | | 001969 | A_{F}^{S}G_{M}^{S}G_{F}A_{M}A_{F}A | | UGUCCAC | | 001952 | A_{M}^{S}A_{F}^{S}A_{M}C_{F}A_{M} | | UGGUGG | |
| | | | | _{M}U_{F}G_{M}U_{F}C_{M}C_{F}A | | CACUGU | | | G_{F}U_{M}G_{F}G_{M}U_{F}G | | ACAUUU | |
| | | | | _{M}C_{F}C_{M}A_{F}C_{M}U_{F}G_{M} | | UU | | | _{M}G_{F}A_{M}C_{F}A_{M}U_{F} | | CCUGA | |
| | | | | U_{F}U_{M}U_{F}-C7OH- | | | | | U_{M}U_{F}C_{M}C_{F}U_{M}^{S} | | | |
| | | | | [_{D}Tx-01-08] | | | | | G_{M}^{S}A_{M}-OH-3′ | | | |
| DT- | 356 | 376 | DTS- | 5′-OH- | 817 | UCCACCA | 1086 | DTS- | 5′-PO4- | 924 | AUGAUG | 1145 |
| 001302 | | | 001970 | U_{F}^{S}C_{M}^{S}C_{F}A_{M}C_{F}C | | CUGUUU | | 001954 | A_{M}^{S}U_{F}^{S}G_{M}A_{F}U_{M} | | AGAAAC | |
| | | | | _{M}A_{F}C_{M}U_{F}G_{M}U_{F}U | | CUCAUCA | | | G_{F}A_{M}G_{F}A_{M}A_{F}A | | AGUGGU | |
| | | | | _{M}U_{F}C_{M}U_{F}C_{M}A_{F}U | | U | | | _{M}C_{F}A_{M}G_{F}U_{M}G_{F} | | GGACA | |
| | | | | _{M}C_{F}^{S}A_{M}^{S}U_{F}- | | | | | G_{M}U_{F}G_{M}G_{F}A_{M}^{S} | | | |
| | | | | C7OH-[_{D}Tx-01- | | | | | C_{M}^{S}A_{M}-OH-3′ | | | |
| | | | | 08] | | | | | | | | |
| DT- | 381 | 401 | DTS- | 5′-OH- | 818 | AAACGA | 977 | DTS- | 5′-PO4- | 925 | ACAGAC | 1125 |
| 001303 | | | 001971 | A_{F}^{S}A_{M}^{S}A_{F}C_{M}G_{F}A | | AUGGCU | | 001956 | A_{M}^{S}C_{F}^{S}A_{M}G_{F}A_{M} | | UGCAGCC | |
| | | | | _{M}A_{F}U_{M}G_{F}G_{M}C_{F}U | | GCAGUC | | | C_{F}U_{M}G_{F}C_{M}A_{F}G | | AUUCGU | |
| | | | | _{M}G_{F}C_{M}A_{F}G_{M}U_{F}C | | UGU | | | _{M}C_{F}C_{M}A_{F}U_{M}U_{F} | | UUGG | |
| | | | | _{M}U_{F}^{S}G_{M}^{S}U_{F}- | | | | | C_{M}G_{F}U_{M}U_{F}U_{M}^{S} | | | |
| | | | | C7OH-[_{D}Tx-01- | | | | | G_{M}^{S}G_{M}-OH-3′ | | | |
| | | | | 08] | | | | | | | | |
| DT- | 405 | 425 | DTS- | 5′-OH- | 784 | GGCCACC | 1067 | DTS- | 5′-PO4- | 926 | AUCGAC | 1141 |
| 001304 | | | 001914 | G_{F}^{S}G_{M}^{S}C_{F}C_{M}^{S}A_{F}C | | AUGAUC | | 001958 | A_{M}^{S}U_{F}^{S}C_{M}G_{F}A_{M} | | AGGAUC | |
| | | | | _{M}C_{F}A_{M}U_{F}G_{M}A_{F}U | | CUGUCG | | | C_{F}A_{M}G_{F}G_{M}A_{F}U | | AUGGUG | |
| | | | | _{M}C_{F}C_{M}U_{F}G_{M}U_{F}C_{M} | | AU | | | _{M}C_{F}A_{M}U_{F}G_{M}G_{F} | | GCCUG | |
| | | | | G_{F}^{S}A_{M}^{S}U_{F}-C7OH- | | | | | U_{M}G_{F}G_{M}C_{F}C_{M}^{S} | | | |
| | | | | [_{D}Tx-01-08] | | | | | U_{M}^{S}G_{M}-OH-3′ | | | |
| DT- | 417 | 437 | DTS- | 5′-OH- | 785 | CCUGUCG | 1021 | DTS- | 5′-PO4- | 927 | AUGCUG | 1147 |
| 001305 | | | 001916 | C_{F}^{S}C_{M}^{S}U_{F}G_{M}U_{F}C | | AUCAUC | | 001960 | A_{M}^{S}U_{F}^{S}G_{M}C_{F}U_{M} | | AAGAUG | |
| | | | | _{M}G_{F}A_{M}U_{F}C_{M}A_{F}U | | UUCAGC | | | G_{F}A_{M}A_{F}G_{M}A_{F}U | | AUCGAC | |
| | | | | _{M}C_{F}U_{M}U_{F}C_{M}A_{F}G | | AU | | | _{M}G_{F}A_{M}U_{F}C_{M}G_{F} | | AGGAU | |
| | | | | _{M}C_{F}^{S}A_{M}^{S}U_{F}- | | | | | A_{M}C_{F}A_{M}G_{F}G_{M}^{S} | | | |
| | | | | C7OH-[_{D}Tx-01- | | | | | A_{M}^{S}U_{M}-OH-3′ | | | |
| | | | | 08] | | | | | | | | |
| DT- | 447 | 467 | DTS- | 5′-OH- | 819 | GUUCCU | 1077 | DTS- | 5′-PO4- | 928 | AGUUGG | 1134 |
| 001306 | | | 001972 | G_{F}^{S}U_{M}^{S}U_{F}C_{M}C_{F}U | | GUUCUU | | 001962 | A_{M}^{S}G_{F}^{S}U_{M}U_{F}G_{M} | | CAGAAG | |
| | | | | _{M}G_{F}U_{M}U_{F}C_{M}U_{F}U | | CUGCCAA | | | G_{F}C_{M}A_{F}G_{M}A_{F}A | | AACAGG | |
| | | | | _{M}C_{F}U_{M}G_{F}C_{M}C_{F}A_{M} | | CU | | | _{M}G_{F}A_{M}A_{F}C_{M}A_{F} | | AACAG | |
| | | | | A_{F}^{S}C_{M}^{S}U_{F}-C7OH- | | | | | G_{M}G_{F}A_{M}A_{F}C_{M}^{S} | | | |
| | | | | [_{D}Tx-01-08] | | | | | A_{M}^{S}G_{M}-OH-3′ | | | |
| DT- | 450 | 470 | DTS- | 5′-OH- | 820 | CCUGUUC | 1022 | DTS- | 5′-PO4- | 929 | AAGAGU | 1117 |
| 001307 | | | 001973 | C_{F}^{S}C_{M}^{S}U_{F}G_{M}U_{F}U | | UUCUGCC | | 001964 | A_{M}^{S}A_{F}^{S}G_{M}A_{F}G_{M} | | UGGCAG | |
| | | | | _{M}C_{F}U_{M}U_{F}C_{M}U_{F}G | | AACUCU | | | U_{F}U_{M}G_{F}G_{M}C_{F}A | | AAGAAC | |
| | | | | _{M}C_{F}C_{M}A_{F}A_{M}C_{F}U_{M} | | U | | | _{M}G_{F}A_{M}A_{F}G_{M}A_{F} | | AGGAA | |
| | | | | C_{F}^{S}U_{M}^{S}U_{F}-C7OH- | | | | | A_{M}C_{F}A_{M}G_{F}G_{M} | | | |
| | | | | [_{D}Tx-01-08] | | | | | A_{M}^{S}A_{M}-OH-3′ | | | |
| DT- | 462 | 482 | DTS- | 5′-OH- | 835 | CCAACUC | 1010 | DTS- | 5′-PO4- | 930 | UUGGUG | 1165 |
| 001322 | | | 002002 | C_{F}^{S}C_{M}^{S}A_{F}A_{M}C_{F}U | | UUCACCC | | 001975 | U_{M}^{S}U_{F}^{S}G_{M}G_{F}U_{M} | | AGGGUG | |
| | | | | _{M}C_{F}U_{M}U_{F}C_{M}A_{F}C_{M} | | UCACCAA | | | G_{F}A_{M}G_{F}G_{M}G_{F}U | | AAGAGU | |
| | | | | C_{F}C_{M}U_{F}C_{M}A_{F}C_{M} | | | | | _{M}G_{F}A_{M}A_{F}G_{M}A_{F} | | UGGCA | |
| | | | | C_{F}^{S}A_{M}^{S}A_{F}-C7OH- | | | | | G_{M}U_{F}U_{M}G_{F}G_{M} | | | |
| | | | | [_{D}Tx-01-08] | | | | | C_{M}^{S}A_{M}-OH-3′ | | | |
| DT- | 484 | 504 | DTS- | 5′-OH- | 836 | GGGGGC | 1071 | DTS- | 5′-PO4- | 931 | AGUGAU | 1133 |
| 001323 | | | 002003 | G_{F}^{S}G_{M}^{S}G_{F}G_{M}G_{F}C | | AGGUUU | | 001977 | A_{M}^{S}G_{F}^{S}U_{M}G_{F}A_{M} | | GUAAAA | |
| | | | | _{M}A_{F}G_{M}G_{F}U_{M}U_{F}U | | UACAUC | | | U_{F}G_{M}U_{F}A_{M}A_{F}A | | CCUGCCC | |
| | | | | _{M}U_{F}A_{M}C_{F}A_{M}U_{F}C | | ACU | | | _{M}A_{F}C_{M}C_{F}U_{M}G_{F} | | CCCU | |
| | | | | _{M}A_{F}^{S}C_{M}^{S}U_{F}- | | | | | C_{M}C_{F}C_{M}C_{F}C_{M}^{S}C | | | |
| | | | | C7OH-[_{D}Tx-01- | | | | | _{M}^{S}U_{M}-OH-3′ | | | |
| | | | | 08] | | | | | | | | |
| DT- | 489 | 509 | DTS- | 5′-OH- | 837 | CAGGUU | 1009 | DTS- | 5′-PO4- | 932 | AUUCCA | 1150 |
| 001324 | | | 002004 | C_{F}^{S}A_{M}^{S}G_{F}G_{M}U_{F}U | | UUACAU | | 001979 | A_{M}^{S}U_{F}^{S}U_{M}C_{F}C_{M} | | GUGAUG | |
| | | | | _{M}U_{F}U_{M}A_{F}C_{M}A_{F}U | | CACUGG | | | A_{F}G_{M}U_{F}G_{M}A_{F}U | | UAAAAC | |
| | | | | _{M}C_{F}A_{M}C_{F}U_{M}G_{F}G | | AAU | | | _{M}G_{F}U_{M}A_{F}A_{M}A_{F} | | CUGCC | |
| | | | | _{M}A_{F}^{S}A_{M}^{S}U_{F}- | | | | | A_{M}C_{F}C_{M}U_{F}G_{M}^{S} | | | |
| | | | | C7OH-[_{D}Tx-01- | | | | | C_{M}^{S}C_{M}-OH-3′ | | | |
| | | | | 08] | | | | | | | | |
| DT- | 492 | 512 | DTS- | 5′-OH- | 838 | GUUUUA | 1081 | DTS- | 5′-PO4- | 933 | AAGAUU | 1119 |
| 001325 | | | 002005 | G_{F}^{S}U_{M}^{S}U_{F}U_{M}U_{F}A | | CAUCACU | | 001981 | A_{M}^{S}A_{F}^{S}G_{M}A_{F}U_{M} | | CCAGUG | |
| | | | | _{M}C_{F}A_{M}U_{F}C_{M}A_{F}C_{M} | | GGAAUC | | | U_{F}C_{M}C_{F}A_{M}G_{F}U | | AUGUAA | |
| | | | | U_{F}G_{M}G_{F}A_{M}A_{F}U_{M} | | UU | | | _{M}G_{F}A_{M}U_{F}G_{M}U_{F} | | AACCU | |
| | | | | C_{F}^{S}U_{M}^{S}U_{F}-C7OH- | | | | | A_{M}A_{F}A_{M}A_{F}C_{M}^{S} | | | |
| | | | | [_{D}Tx-01-08] | | | | | C_{M}^{S}U_{M}-OH-3′ | | | |
| DT- | 499 | 519 | DTS- | 5′-OH- | 839 | AUCACU | 997 | DTS- | 5′-PO4- | 934 | AAUUUG | 1124 |
| 001326 | | | 002006 | A_{F}^{S}U_{M}^{S}C_{F}A_{M}C_{F}U | | GGAAUC | | 001983 | A_{M}^{S}A_{F}^{S}U_{M}U_{F}U_{M} | | GAAGAU | |
| | | | | _{M}G_{F}G_{M}A_{F}A_{M}U_{F}C | | UUCCAA | | | G_{F}G_{M}A_{F}A_{M}G_{F}A | | UCCAGU | |
| | | | | _{M}U_{F}U_{M}C_{F}C_{M}A_{F}A | | AUU | | | _{M}U_{F}U_{M}C_{F}C_{M}A_{F} | | GAUGU | |
| | | | | _{M}A_{F}^{S}U_{M}^{S}U_{F}- | | | | | G_{M}U_{F}G_{M}A_{F}U_{M}^{S} | | | |
| | | | | C7OH-[_{D}Tx-01- | | | | | G_{M}^{S}U_{M}-OH-3′ | | | |
| | | | | 08] | | | | | | | | |
| DT- | 505 | 525 | DTS- | 5′-OH- | 840 | GGAAUC | 1063 | DTS- | 5′-PO4- | 935 | AGCAAG | 1130 |
| 001327 | | | 002007 | G_{F}^{S}G_{M}^{S}A_{F}A_{M}U_{F}C | | UUCCAA | | 001985 | A_{M}^{S}G_{F}^{S}C_{M}A_{F}A_{M} | | AAUUUG | |
| | | | | _{M}U_{F}U_{M}C_{F}C_{M}A_{F}A | | AUUCUU | | | G_{F}A_{M}A_{F}U_{M}U_{F}U | | GAAGAU | |
| | | | | _{M}A_{F}U_{M}U_{F}C_{M}U_{F}U | | GCU | | | _{M}G_{F}G_{M}A_{F}A_{M}G_{F} | | UCCAG | |
| | | | | _{M}G_{F}^{S}C_{M}^{S}U_{F}- | | | | | A_{M}U_{F}U_{M}C_{F}C_{M} | | | |
| | | | | C7OH-[_{D}Tx-01- | | | | | A_{M}^{S}G_{M}-OH-3′ | | | |
| | | | | 08] | | | | | | | | |
| DT- | 534 | 554 | DTS- | 5′-OH- | 841 | CGUGAU | 1029 | DTS- | 5′-PO4- | 936 | AUGGCC | 1148 |
| 001328 | | | 002008 | C_{F}^{S}G_{M}^{S}U_{F}G_{M}A_{F}U | | GAGUGC | | 001987 | A_{M}^{S}U_{F}^{S}G_{M}G_{F}C_{M} | | GCAGCAC | |
| | | | | _{M}G_{F}A_{M}G_{F}U_{M}G_{F}C | | UGCGGCC | | | C_{F}G_{M}C_{F}A_{M}G_{F}C | | UCAUCAC | |
| | | | | _{M}U_{F}G_{M}C_{F}G_{M}G_{F}C | | AU | | | _{M}A_{F}C_{M}U_{F}C_{M}A_{F} | | GCA | |
| | | | | _{M}C_{F}^{S}A_{M}^{S}U_{F}- | | | | | U_{M}C_{F}A_{M}C_{F}G_{M}^{S} | | | |
| | | | | C7OH-[_{D}Tx-01- | | | | | C_{M}^{S}A_{M}-OH-3′ | | | |
| | | | | 08] | | | | | | | | |
| DT- | 567 | 587 | DTS- | 5′-OH- | 842 | GCACCCG | 1056 | DTS- | 5′-PO4- | 937 | UUGAGA | 1163 |
| 001329 | | | 002009 | G_{F}^{S}C_{M}^{S}A_{F}C_{M}C_{F}C | | GAGUGG | | 001989 | U_{M}^{S}U_{F}^{S}G_{M}A_{F}G_{M} | | UGCCACU | |
| | | | | _{M}G_{F}G_{M}A_{F}G_{M}U_{F}G | | CAUCUCA | | | A_{F}U_{M}G_{F}C_{M}C_{F}A | | CCGGGU | |
| | | | | _{M}G_{F}C_{M}A_{F}U_{M}C_{F}U | | A | | | _{M}C_{F}U_{M}C_{F}C_{M}G_{F} | | GCCU | |
| | | | | _{M}C_{F}^{S}A_{M}^{S}A_{F}- | | | | | G_{M}G_{F}U_{M}G_{F}C_{M}^{S} | | | |
| | | | | C7OH-[_{D}Tx-01- | | | | | C_{M}^{S}U_{M}-OH-3′ | | | |
| | | | | 08] | | | | | | | | |
| DT- | 588 | 608 | DTS- | 5′-OH- | 843 | CUCGGA | 1039 | DTS- | 5′-PO4- | 938 | AAACCG | 1113 |
| 001330 | | | 002010 | C_{F}^{S}U_{M}^{S}C_{F}G_{M}G_{F}A | | UUACUCC | | 001991 | A_{M}^{S}A_{F}^{S}A_{M}C_{F}C_{M} | | UAGGAG | |
| | | | | _{M}U_{F}U_{M}A_{F}C_{M}U_{F}C | | UACGGU | | | G_{F}U_{M}A_{F}G_{M}G_{F}A | | UAAUCC | |
| | | | | _{M}C_{F}U_{M}A_{F}C_{M}G_{F}G | | UU | | | _{M}G_{F}U_{M}A_{F}A_{M}U_{F} | | GAGUU | |
| | | | | _{M}U_{F}^{S}U_{M}^{S}U_{F}- | | | | | C_{M}C_{F}G_{M}A_{F}G_{M} | | | |
| | | | | C7OH-[_{D}Tx-01- | | | | | U_{M}^{S}U_{M}-OH-3′ | | | |
| | | | | 08] | | | | | | | | |
| DT- | 597 | 617 | DTS- | 5′-OH- | 844 | CUCCUAC | 1033 | DTS- | 5′-PO4- | 939 | AUGUAG | 1149 |
| 001331 | | | 002011 | C_{F}^{S}U_{M}^{S}C_{F}C_{M}U_{F}A | | GGUUUC | | 001993 | A_{M}^{S}U_{F}^{S}G_{M}U_{F}A_{M} | | GCGAAA | |
| | | | | _{M}C_{F}G_{M}G_{F}U_{M}U_{F}U | | GCCUACA | | | G_{F}G_{M}C_{F}G_{M}A_{F}A | | CCGUAG | |
| | | | | _{M}C_{F}G_{M}C_{F}C_{M}U_{F}A_{M} | | U | | | _{M}A_{F}C_{M}C_{F}G_{M}U_{F} | | GAGUA | |
| | | | | C_{F}^{S}A_{M}^{S}U_{F}-C7OH- | | | | | A_{M}G_{F}G_{M}A_{F}G^{S} | | | |
| | | | | [_{D}Tx-01-08] | | | | | U_{M}^{S}A_{M}-OH-3′ | | | |
| DT- | 600 | 620 | DTS- | 5′-OH- | 845 | CUACGG | 1031 | DTS- | 5′-PO4- | 940 | AGGAUG | 1132 |
| 001332 | | | 002012 | C_{F}^{S}U_{M}^{S}A_{F}C_{M}G_{F}G | | UUUCGCC | | 001995 | A_{M}^{S}G_{F}^{S}G_{M}A_{F}U_{M} | | UAGGCG | |
| | | | | _{M}U_{F}U_{M}U_{F}C_{M}G_{F}C | | UACAUCC | | | G_{F}U_{M}A_{F}G_{M}G_{F}C | | AAACCG | |
| | | | | _{M}C_{F}U_{M}A_{F}C_{M}A_{F}U | | U | | | _{M}G_{F}A_{M}A_{F}A_{M}C_{F} | | UAGGA | |
| | | | | _{M}C_{F}^{S}C_{M}^{S}U_{F}- | | | | | C_{M}G_{F}U_{M}A_{F}G_{M} | | | |
| | | | | C7OH-[_{D}Tx-01- | | | | | G_{M}^{S}A_{M}-OH-3′ | | | |
| | | | | 08] | | | | | | | | |
| DT- | 651 | 671 | DTS- | 5′-OH- | 846 | CAGCGG | 1008 | DTS- | 5′-PO4- | 941 | AUCACA | 1139 |
| 001333 | | | 002013 | C_{F}^{S}A_{M}^{S}G_{F}C_{M}G_{F}G | | UGUCAU | | 001997 | A_{M}^{S}U_{F}^{S}C_{M}A_{F}C_{M} | | UAGAUG | |
| | | | | _{M}U_{F}G_{M}U_{F}C_{M}A_{F}U | | CUAUGU | | | A_{F}U_{M}A_{F}G_{M}A_{F}U | | ACACCGC | |
| | | | | _{M}C_{F}U_{M}A_{F}U_{M}G_{F}U | | GAU | | | _{M}G_{F}A_{M}C_{F}A_{M}C_{F} | | UGAG | |
| | | | | _{M}G_{F}^{S}A_{M}^{S}U_{F}- | | | | | C_{M}G_{F}C_{M}U_{F}G_{M}^{S} | | | |
| | | | | C7OH-[_{D}Tx-01- | | | | | A_{M}^{S}G_{M}-OH-3′ | | | |
| | | | | 08] | | | | | | | | |
| DT- | 654 | 674 | DTS- | 5′-OH- | 847 | CGGUGU | 1026 | DTS- | 5′-PO4- | 942 | AAGAUC | 1118 |
| 001334 | | | 002014 | C_{F}^{S}G_{M}^{S}G_{F}U_{M}G_{F}U | | CAUCUA | | 001999 | A_{M}^{S}A_{F}^{S}G_{M}A_{F}U_{M} | | ACAUAG | |
| | | | | _{M}C_{F}A_{M}U_{F}C_{M}U_{F}A | | UGUGAU | | | C_{F}A_{M}C_{F}A_{M}U_{F}A | | AUGACA | |
| | | | | _{M}U_{F}G_{M}U_{F}G_{M}A_{F}U | | CUU | | | _{M}G_{F}A_{M}U_{F}G_{M}A_{F} | | CCGCU | |
| | | | | _{M}C_{F}^{S}U_{M}^{S}U_{F}- | | | | | C_{M}A_{F}C_{M}C_{F}G_{M}^{S}C | | | |
| | | | | C7OH-[_{D}Tx-01- | | | | | _{M}^{S}U_{M}-OH-3′ | | | |
| | | | | 08] | | | | | | | | |
| DT- | 661 | 681 | DTS- | 5′-OH- | 848 | AUCUAU | 999 | DTS- | 5′-PO4- | 943 | UUUCCGC | 1166 |
| 001335 | | | 002015 | A_{F}^{S}U_{M}^{S}C_{F}U_{M}A_{F}U | | GUGAUC | | 002001 | U_{M}^{S}U_{F}^{S}U_{M}C_{F}C_{M} | | AAGAUC | |
| | | | | _{M}G_{F}U_{M}G_{F}A_{M}U_{F}C | | UUGCGG | | | G_{F}C_{M}A_{F}A_{M}G_{F}A | | ACAUAG | |
| | | | | _{M}U_{F}U_{M}G_{F}C_{M}G_{F}G | | AAA | | | _{M}U_{F}C_{M}A_{F}C_{M}A_{F} | | AUGA | |
| | | | | _{M}A_{F}^{S}A_{M}^{S}A_{F}- | | | | | U_{M}A_{F}G_{M}A_{F}U_{M} | | | |
| | | | | C7OH-[_{D}Tx-01- | | | | | G_{M}^{S}A_{M}-OH-3′ | | | |
| | | | | 08] | | | | | | | | |
| DT- | 783 | 803 | DTS- | 5′-OH- | 857 | AAAUCCC | 979 | DTS- | 5′-PO4- | 944 | UUUGGU | 1169 |
| 001344 | | | 002032 | A_{F}^{S}A_{M}^{S}A_{F}U_{M}C_{F}C | | AAACUC | | 002017 | U_{M}^{S}U_{F}^{S}U_{M}G_{F}G_{M} | | UUGAGU | |
| | | | | _{M}C_{F}A_{M}A_{F}A_{M}C_{F}U | | AAACCA | | | U_{F}U_{M}U_{F}G_{M}A_{F}G | | UUGGGA | |
| | | | | _{M}C_{F}A_{M}A_{F}A_{M}C_{F}C_{M} | | AA | | | _{M}U_{F}U_{M}U_{F}G_{M}G_{F} | | UUUUG | |
| | | | | [_{D}Tx-01-08] | | | | | G_{M}A_{F}U_{M}U_{F}U_{M} | | | |
| | | | | | | | | | U_{M}^{S}G_{M}-OH-3′ | | | |
| DT- | 832 | 852 | DTS- | 5′-OH- | 858 | UGCUGU | 1098 | DTS- | 5′-PO4- | 945 | AUACAU | 1137 |
| 001345 | | | 002033 | U_{F}^{S}G_{M}^{S}C_{F}U_{M}G_{F}U | | UGAUUG | | 002019 | A_{M}^{S}U_{F}^{S}A_{M}C_{F}A_{M} | | CUUCAA | |
| | | | | _{M}U_{F}G_{M}A_{F}U_{M}U_{F}G | | AAGAUG | | | U_{F}C_{M}U_{F}U_{M}C_{F}A | | UCAACA | |
| | | | | _{M}A_{F}A_{M}G_{F}A_{M}U_{F}G | | UAU | | | _{M}A_{F}U_{M}C_{F}A_{M}A_{F} | | GCAAC | |
| | | | | _{M}U_{F}^{S}A_{M}^{S}U_{F}- | | | | | C_{M}A_{F}G_{M}C_{F}A_{M}^{S} | | | |
| | | | | C7OH-[_{D}Tx-01- | | | | | A_{M}^{S}C_{M}-OH-3′ | | | |
| | | | | 08] | | | | | | | | |
| DT- | 863 | 883 | DTS- | 5′-OH- | 859 | CGGUUU | 1027 | DTS- | 5′-PO4- | 946 | AUAAAU | 1135 |
| 001346 | | | 002034 | C_{F}^{S}G_{M}^{S}G_{F}U_{M}U_{F}U | | AUAAAA | | 002021 | A_{M}^{S}U_{F}^{S}A_{M}A_{F}A_{M} | | AGGUUU | |
| | | | | _{M}A_{F}U_{M}A_{F}A_{M}A_{F}A | | CCUAUU | | | U_{F}A_{M}G_{F}G_{M}U_{F}U | | UAUAAA | |
| | | | | _{M}C_{F}C_{M}U_{F}A_{M}U_{F}U | | UAU | | | _{M}U_{F}U_{M}A_{F}U_{M}A_{F} | | CCGGA | |
| | | | | _{M}U_{F}^{S}A_{M}^{S}U_{F}- | | | | | A_{M}A_{F}C_{M}C_{F}G^{S} | | | |
| | | | | C7OH-[_{D}Tx-01- | | | | | G_{M}^{S}A_{M}-OH-3′ | | | |
| | | | | 08] | | | | | | | | |
| DT- | 902 | 922 | DTS- | 5′-OH- | 860 | GUACAU | 1073 | DTS- | 5′-PO4- | 947 | AAAGCA | 1114 |
| 001347 | | | 002035 | G_{F}^{S}U_{M}^{S}A_{F}C_{M}A_{F}U | | AGUAUU | | 002023 | A_{M}^{S}A_{F}^{S}A_{M}G_{F}C_{M} | | AACAAU | |
| | | | | _{M}A_{F}G_{M}U_{F}A_{M}U_{F}U | | GUUUGC | | | A_{F}A_{M}A_{F}C_{M}A_{F}A | | ACUAUG | |
| | | | | _{M}G_{F}U_{M}U_{F}U_{M}G_{F}C | | UUU | | | _{M}U_{F}A_{M}C_{F}U_{M}A_{F} | | UACAU | |
| | | | | _{M}U_{F}^{S}U_{M}^{S}U_{F}- | | | | | U_{M}G_{F}U_{M}A_{F}C^{S} | | | |
| | | | | C7OH-[_{D}Tx-01- | | | | | A_{M}^{S}U_{M}-OH-3′ | | | |
| | | | | 08] | | | | | | | | |
| DT- | 927 | 947 | DTS- | 5′-OH- | 861 | GUUGAC | 1078 | DTS- | 5′-PO4- | 948 | AACACG | 1116 |
| 001348 | | | 002036 | G_{F}^{S}U_{M}^{S}U_{F}G_{M}A_{F}C | | CAUCAGC | | 002025 | A_{M}^{S}A_{F}^{S}C_{M}A_{F}C_{M} | | AGGCUG | |
| | | | | _{M}C_{F}A_{M}U_{F}C_{M}A_{F}G | | CUCGUG | | | G_{F}A_{M}G_{F}G_{M}C_{F}U | | AUGGUC | |
| | | | | _{M}C_{F}C_{M}U_{F}C_{M}G_{F}U_{M} | | UU | | | _{M}G_{F}A_{M}U_{F}G_{M}G_{F} | | AACAU | |
| | | | | G_{F}^{S}U_{M}^{S}U_{F}-C7OH- | | | | | U_{M}C_{F}A_{M}A_{F}C_{M}^{S} | | | |
| | | | | [_{D}Tx-01-08] | | | | | A_{M}^{S}U_{M}-OH-3′ | | | |
| DT- | 956 | 976 | DTS- | 5′-OH- | 862 | AAGAAG | 981 | DTS- | 5′-PO4- | 949 | AAAGUU | 1115 |
| 001349 | | | 002037 | A_{F}^{S}A_{M}^{S}G_{F}A_{M}A_{F}G | | UAGCUA | | 002027 | A_{M}^{S}A_{F}^{S}A_{M}G_{F}U_{M} | | CCUUAGC | |
| | | | | _{M}U_{F}A_{M}G_{F}C_{M}U_{F}A | | AGGAAC | | | U_{F}C_{M}C_{F}U_{M}U_{F}A | | UACUUC | |
| | | | | _{M}A_{F}G_{M}G_{F}A_{M}A_{F}C | | UUU | | | _{M}G_{F}C_{M}U_{F}A_{M}C_{F} | | UUUA | |
| | | | | _{M}U_{F}^{S}U_{M}^{S}U_{F}- | | | | | U_{M}U_{F}C_{M}U_{F}U_{M}^{S} | | | |
| | | | | C7OH-[_{D}Tx-01- | | | | | U_{M}^{S}A_{M}-OH-3′ | | | |
| | | | | 08] | | | | | | | | |
| DT- | 965 | 985 | DTS- | 5′-OH- | 863 | CUAAGG | 1030 | DTS- | 5′-PO4- | 950 | UUAGGA | 1159 |
| 001350 | | | 002038 | C_{F}^{S}U_{M}^{S}A_{F}A_{M}G_{F}G | | AACUUU | | 002029 | U_{M}^{S}U_{F}^{S}A_{M}G_{F}G_{M} | | UGUAAA | |
| | | | | _{M}A_{F}A_{M}C_{F}U_{M}U_{F}U | | ACAUCCU | | | A_{F}U_{M}G_{F}U_{M}A_{F}A | | GUUCCU | |
| | | | | _{M}A_{F}C_{M}A_{F}U_{M}C_{F}C_{M} | | AA | | | _{M}A_{F}G_{M}U_{F}U_{M}C_{E} | | UAGCU | |
| | | | | U_{F}^{S}A_{M}^{S}A_{F}-C7OH- | | | | | C_{M}U_{F}U_{M}A_{F}G^{S} | | | |
| | | | | [_{D}Tx-01-08] | | | | | C_{M}^{S}U_{M}-OH-3′ | | | |
| DT- | 1784 | 1804 | DTS- | 5′-OH- | 864 | ACUGUG | 992 | DTS- | 5′-PO4- | 951 | AUGCAU | 1146 |
| 001351 | | | 002039 | A_{F}^{S}C_{M}^{S}U_{F}G_{M}U_{F}G | | UGGACU | | 002031 | A_{M}^{S}U_{F}^{S}G_{M}C_{F}A_{M} | | CUUAGU | |
| | | | | _{M}U_{F}G_{M}G_{F}A_{M}C_{F}U | | AAGAUG | | | U_{F}C_{M}U_{F}U_{M}A_{F}G | | CCACACA | |
| | | | | _{M}A_{F}A_{M}G_{F}A_{M}U_{F}G | | CAU | | | _{M}U_{F}C_{M}C_{F}A_{M}C_{E} | | GUUG | |
| | | | | _{M}C_{F}^{S}A_{M}^{S}U_{F}- | | | | | A_{M}C_{F}A_{M}G_{F}U_{M}^{S} | | | |
| | | | | C7OH-[_{D}Tx-01- | | | | | U_{M}^{S}G_{M}-OH-3′ | | | |
| | | | | 08] | | | | | | | | |
| DT- | 160 | 180 | DTS- | 5′-OH- | 868 | CGCUGU | 1024 | DTS- | 5′-PO4- | 952 | UUUCUG | 1167 |
| 001355 | | | 002046 | C_{F}^{S}G_{M}^{S}C_{F}U_{M}G_{F}U | | UUGGCC | | 002041 | U_{M}^{S}U_{F}^{S}U_{M}C_{F}U_{M} | | CCCGGCC | |
| | | | | _{M}U_{F}U_{M}G_{F}G_{M}C_{F}C | | GGGCAG | | | G_{F}C_{M}C_{F}C_{M}G_{F}G | | AAACAG | |
| | | | | _{M}G_{F}G_{M}G_{F}C_{M}A_{F}G | | AAA | | | _{M}C_{F}C_{M}A_{F}A_{M}A_{F} | | CGUA | |
| | | | | _{M}A_{F}^{S}A_{M}^{S}A_{F}- | | | | | C_{M}A_{F}G_{M}C_{F}G_{M} | | | |
| | | | | C7OH-[_{D}Tx-01- | | | | | U_{M}^{S}A_{M}-OH-3′ | | | |
| | | | | 08] | | | | | | | | |
| DT- | 175 | 195 | DTS- | 5′-OH- | 869 | CAGAAA | 1007 | DTS- | 5′-PO4- | 953 | UUCUGC | 1162 |
| 001356 | | | 002047 | C_{F}^{S}A_{M}^{S}G_{F}A_{M}A_{F}A | | CUCCGCU | | 002043 | U_{M}^{S}U_{F}^{S}C_{M}U_{F}G_{M} | | UCAGCG | |
| | | | | _{M}C_{F}U_{M}C_{F}C_{M}G_{F}C_{M} | | GAGCAG | | | C_{F}U_{M}C_{F}A_{M}G_{F}C | | GAGUUU | |
| | | | | U_{F}G_{M}A_{F}G_{M}C_{F}A_{M} | | AA | | | _{M}G_{F}G_{M}A_{F}G_{M}U_{F} | | CUGCC | |
| | | | | G_{F}^{S}A_{M}^{S}A_{F}-C7OH- | | | | | U_{M}U_{F}C_{M}U_{F}G^{S} | | | |
| | | | | [_{D}Tx-01-08] | | | | | C_{M}^{S}C_{M}-OH-3′ | | | |
| DT- | 178 | 198 | DTS- | 5′-OH- | 870 | AAACUCC | 978 | DTS- | 5′-PO4- | 954 | AAGUUC | 1120 |
| 001357 | | | 002048 | A_{F}^{S}A_{M}^{S}A_{F}C_{M}U_{F}C | | GCUGAG | | 002045 | A_{M}^{S}A_{F}^{S}G_{M}U_{F}U_{M} | | UGCUCA | |
| | | | | _{M}C_{F}G_{M}C_{F}U_{M}G_{F}A | | CAGAAC | | | C_{F}U_{M}G_{F}C_{M}U_{F}C | | GCGGAG | |
| | | | | _{M}G_{F}C_{M}A_{F}G_{M}A_{F}A | | UU | | | _{M}A_{F}G_{M}C_{F}G_{M}G_{F} | | UUUCU | |
| | | | | _{M}C_{F}^{S}U_{M}^{S}U_{F}- | | | | | A_{M}G_{F}U_{M}U_{F}U_{M}^{S} | | | |
| | | | | C7OH-[_{D}Tx-01- | | | | | C_{M}^{S}U_{M}-OH-3′ | | | |
| | | | | 08] | | | | | | | | |
| DT- | 243 | 263 | DTS- | 5′-OH- | 813 | CGUCGCG | 1028 | DTS- | 5′-VP- | 955 | AGCAGC | 1131 |
| 001358 | | | 001966 | C_{F}^{S}G_{M}^{S}U_{F}C_{M}G_{F}C | | GUGCUG | | 002049 | A_{M}^{S}G_{F}^{S}C_{M}A_{F}G_{M} | | ACCAGCA | |
| | | | | _{M}G_{F}G_{M}U_{F}G_{M}C_{F}U | | GUGCUG | | | C_{F}A_{M}C_{F}C_{M}A_{F}G | | CCGCGAC | |
| | | | | _{M}G_{F}G_{M}U_{F}G_{M}C_{F}U | | CU | | | _{M}C_{F}A_{M}C_{F}C_{M}G_{F}C | | GUG | |
| | | | | _{M}G_{F}^{S}C_{M}^{S}U_{F}- | | | | | _{M}G_{F}A_{M}C_{F}G_{M}^{S}U | | | |
| | | | | C7OH-[_{D}Tx-01- | | | | | _{M}^{S}G_{M}-OH-3′ | | | |
| | | | | 08] | | | | | | | | |
| DT- | 251 | 271 | DTS- | 5′-OH- | 814 | UGCUGG | 1097 | DTS- | 5′-VP- | 956 | AGACGA | 1128 |
| 001359 | | | 001967 | U_{F}^{S}G_{M}^{S}C_{F}U_{M}G_{F}G | | UGCUGC | | 002050 | A_{M}^{S}G_{F}^{S}A_{M}C_{F}G_{M} | | ACAGCA | |
| | | | | _{M}U_{F}G_{M}C_{F}U_{M}G_{F}C | | UGUUCG | | | A_{F}A_{M}C_{F}A_{M}G_{F}C | | GCACCAG | |
| | | | | _{M}U_{F}G_{M}U_{F}U_{M}C_{F}G | | UCU | | | _{M}A_{F}G_{M}C_{F}A_{M}C_{F} | | CACC | |
| | | | | _{M}U_{F}^{S}C_{M}^{S}U_{F}- | | | | | C_{M}A_{F}G_{M}C_{F}A_{M}^{S} | | | |
| | | | | C7OH-[_{D}Tx-01- | | | | | C_{M}^{S}C_{M}-OH-3′ | | | |
| | | | | 08] | | | | | | | | |
| DT- | 348 | 368 | DTS- | 5′-OH- | 816 | AGGAAA | 994 | DTS- | 5′-VP- | 957 | AAACAG | 1112 |
| 001360 | | | 001969 | A_{F}^{S}G_{M}^{S}G_{F}A_{M}A_{F}A | | UGUCCAC | | 002051 | A_{M}^{S}A_{F}^{S}A_{M}C_{F}A_{M} | | UGGUGG | |
| | | | | _{M}U_{F}G_{M}U_{F}C_{M}C_{F}A | | CACUGU | | | G_{F}U_{M}G_{F}G_{M}U_{F}G | | ACAUUU | |
| | | | | _{M}C_{F}C_{M}A_{F}C_{M}U_{F}G_{M} | | UU | | | _{M}G_{F}A_{M}C_{F}A_{M}U_{F} | | CCUGA | |
| | | | | U_{F}U_{M}U_{F}-C7OH- | | | | | U_{M}U_{F}C_{M}C_{F}U_{M}^{S} | | | |
| | | | | [_{D}Tx-01-08] | | | | | G_{M}^{S}A_{M}-OH-3′ | | | |
| DT- | 356 | 376 | DTS- | 5′-OH- | 817 | UCCACCA | 1086 | DTS- | 5′-VP- | 958 | AUGAUG | 1145 |
| 001361 | | | 001970 | U_{F}^{S}C_{M}^{S}C_{F}A_{M}C_{F}C | | CUGUUU | | 002052 | A_{M}^{S}U_{F}^{S}G_{M}A_{F}U_{M} | | AGAAAC | |
| | | | | _{M}A_{F}C_{M}U_{F}G_{M}U_{F}U | | CUCAUCA | | | G_{F}A_{M}G_{F}A_{M}A_{F}A | | AGUGGU | |
| | | | | _{M}U_{F}C_{M}U_{F}C_{M}A_{F}U | | U | | | _{M}C_{F}A_{M}G_{F}U_{M}G_{F} | | GGACA | |
| | | | | _{M}C_{F}^{S}A_{M}^{S}U_{F}- | | | | | G_{M}U_{F}G_{M}G_{F}A_{M}^{S} | | | |
| | | | | C7OH-[_{D}Tx-01- | | | | | C_{M}^{S}A_{M}-OH-3′ | | | |
| | | | | 08] | | | | | | | | |
| DT- | 381 | 401 | DTS- | 5′-OH- | 818 | AAACGA | 977 | DTS- | 5′-VP- | 959 | ACAGAC | 1125 |
| 001362 | | | 001971 | A_{F}^{S}A_{M}^{S}A_{F}C_{M}G_{F}A | | AUGGCU | | 002053 | A_{M}^{S}C_{F}^{S}A_{M}G_{F}A_{M} | | UGCAGCC | |
| | | | | _{M}A_{F}U_{M}G_{F}G_{M}C_{F}U | | GCAGUC | | | C_{F}U_{M}G_{F}C_{M}A_{F}G | | AUUCGU | |
| | | | | _{M}G_{F}C_{M}A_{F}G_{M}U_{F}C | | UGU | | | _{M}C_{F}C_{M}A_{F}U_{M}U_{F} | | UUGG | |
| | | | | _{M}U_{F}^{S}G_{M}^{S}U_{F}- | | | | | C_{M}G_{F}U_{M}U_{F}U_{M}^{S} | | | |
| | | | | C7OH-[_{D}Tx-01- | | | | | G_{M}^{S}G_{M}-OH-3′ | | | |
| | | | | 08] | | | | | | | | |
| DT- | 450 | 470 | DTS- | 5′-OH- | 820 | CCUGUUC | 1022 | DTS- | 5′-VP- | 960 | AAGAGU | 1117 |
| 001363 | | | 001973 | C_{F}^{S}C_{M}^{S}U_{F}G_{M}U_{F}U | | UUCUGCC | | 002054 | A_{M}^{S}A_{F}^{S}G_{M}A_{F}G_{M} | | UGGCAG | |
| | | | | _{M}C_{F}U_{M}U_{F}C_{M}U_{F}G | | AACUCU | | | U_{F}U_{M}G_{F}G_{M}C_{F}A | | AAGAAC | |
| | | | | _{M}C_{F}C_{M}A_{F}A_{M}C_{F}U_{M} | | U | | | _{M}G_{F}A_{M}A_{F}G_{M}A_{F} | | AGGAA | |
| | | | | C_{F}^{S}U_{M}^{S}U_{F}-C7OH- | | | | | A_{M}C_{F}A_{M}G_{F}G^{S} | | | |
| | | | | [_{D}Tx-01-08] | | | | | A_{M}^{S}A_{M}-OH-3′ | | | |
| DT- | 462 | 482 | DTS- | 5′-OH- | 835 | CCAACUC | 1010 | DTS- | 5′-VP- | 961 | UUGGUG | 1165 |
| 001364 | | | 002002 | C_{F}^{S}C_{M}^{S}A_{F}A_{M}C_{F}U | | UUCACCC | | 002055 | U_{M}^{S}U_{F}^{S}G_{M}G_{F}U_{M} | | AGGGUG | |
| | | | | _{M}C_{F}U_{M}U_{F}C_{M}A_{F}C_{M} | | UCACCAA | | | G_{F}A_{M}G_{F}G_{M}G_{F}U | | AAGAGU | |
| | | | | C_{F}C_{M}U_{F}C_{M}A_{F}C_{M} | | | | | _{M}G_{F}A_{M}A_{F}G_{M}A_{F} | | UGGCA | |
| | | | | C_{F}^{S}A_{M}^{S}A_{F}-C7OH- | | | | | G_{M}U_{F}U_{M}G_{F}G^{S} | | | |
| | | | | [_{D}Tx-01-08] | | | | | C_{M}^{S}A_{M}-OH-3′ | | | |
| DT- | 484 | 504 | DTS- | 5′-OH- | 836 | GGGGGC | 1071 | DTS- | 5′-VP- | 962 | AGUGAU | 1133 |
| 001365 | | | 002003 | G_{F}^{S}G_{M}^{S}G_{F}G_{M}G_{F}C | | AGGUUU | | 002056 | A_{M}^{S}G_{F}^{S}U_{M}G_{F}A_{M} | | GUAAAA | |
| | | | | _{M}A_{F}G_{M}G_{F}U_{M}U_{F}U | | UACAUC | | | U_{F}G_{M}U_{F}A_{M}A_{F}A | | CCUGCCC | |
| | | | | _{M}U_{F}A_{M}C_{F}A_{M}U_{F}C | | ACU | | | _{M}A_{F}C_{M}C_{F}U_{M}G_{F} | | CCCU | |
| | | | | _{M}A_{F}^{S}C_{M}^{S}U_{F}- | | | | | C_{M}C_{F}C_{M}C_{F}C_{M}^{S}C | | | |
| | | | | C7OH-[_{D}Tx-01- | | | | | _{M}^{S}U_{M}-OH-3′ | | | |
| | | | | 08] | | | | | | | | |
| DT- | 489 | 509 | DTS- | 5′-OH- | 837 | CAGGUU | 1009 | DTS- | 5′-VP- | 963 | AUUCCA | 1150 |
| 001366 | | | 002004 | C_{F}^{S}A_{M}^{S}G_{F}G_{M}U_{F}U | | UUACAU | | 002057 | A_{M}^{S}U_{F}^{S}U_{M}C_{F}C_{M} | | GUGAUG | |
| | | | | _{M}U_{F}U_{M}A_{F}C_{M}A_{F}U | | CACUGG | | | A_{F}G_{M}U_{F}G_{M}A_{F}U | | UAAAAC | |
| | | | | _{M}C_{F}A_{M}C_{F}U_{M}G_{F}G | | AAU | | | _{M}G_{F}U_{M}A_{F}A_{M}A_{F} | | CUGCC | |
| | | | | _{M}A_{F}^{S}A_{M}^{S}U_{F}- | | | | | A_{M}C_{F}C_{M}U_{F}G^{S} | | | |
| | | | | C7OH-[_{D}Tx-01- | | | | | C_{M}^{S}C_{M}-OH-3′ | | | |
| | | | | 08] | | | | | | | | |
| DT- | 492 | 512 | DTS- | 5′-OH- | 838 | GUUUUA | 1081 | DTS- | 5′-VP- | 964 | AAGAUU | 1119 |
| 001367 | | | 002005 | G_{F}^{S}U_{M}^{S}U_{F}U_{M}U_{F}A | | CAUCACU | | 002058 | A_{M}^{S}A_{F}^{S}G_{M}A_{F}U_{M} | | CCAGUG | |
| | | | | _{M}C_{F}A_{M}U_{F}C_{M}A_{F}C_{M} | | GGAAUC | | | U_{F}C_{M}C_{F}A_{M}G_{F}U | | AUGUAA | |
| | | | | U_{F}G_{M}G_{F}A_{M}A_{F}U_{M} | | UU | | | _{M}G_{F}A_{M}U_{F}G_{M}U_{F} | | AACCU | |
| | | | | C_{F}^{S}U_{M}^{S}U_{F}-C7OH- | | | | | A_{M}A_{F}A_{M}A_{F}C_{M}^{S} | | | |
| | | | | [_{D}Tx-01-08] | | | | | C_{M}^{S}U_{M}-OH-3′ | | | |
| DT- | 534 | 554 | DTS- | 5′-OH- | 841 | CGUGAU | 1029 | DTS- | 5′-VP- | 965 | AUGGCC | 1148 |
| 001368 | | | 002008 | C_{F}^{S}G_{M}^{S}U_{F}G_{M}A_{F}U | | GAGUGC | | 002059 | A_{M}^{S}U_{F}^{S}G_{M}G_{F}C_{M} | | GCAGCAC | |
| | | | | _{M}G_{F}A_{M}G_{F}U_{M}G_{F}C | | UGCGGCC | | | C_{F}G_{M}C_{F}A_{M}G_{F}C | | UCAUCAC | |
| | | | | _{M}U_{F}G_{M}C_{F}G_{M}G_{F}C | | AU | | | _{M}A_{F}C_{M}U_{F}C_{M}A_{F} | | GCA | |
| | | | | _{M}C_{F}^{S}A_{M}^{S}U_{F}- | | | | | U_{M}C_{F}A_{M}C_{F}G_{M}^{S} | | | |
| | | | | C7OH-[_{D}Tx-01- | | | | | C_{M}^{S}A_{M}-OH-3′ | | | |
| | | | | 08] | | | | | | | | |
| DT- | 588 | 608 | DTS- | 5′-OH- | 843 | CUCGGA | 1039 | DTS- | 5′-VP- | 966 | AAACCG | 1113 |
| 001369 | | | 002010 | C_{F}^{S}U_{M}^{S}C_{F}G_{M}G_{F}A | | UUACUCC | | 002060 | A_{M}^{S}A_{F}^{S}A_{M}C_{F}C_{M} | | UAGGAG | |
| | | | | _{M}U_{F}U_{M}A_{F}C_{M}U_{F}C | | UACGGU | | | G_{F}U_{M}A_{F}G_{M}G_{F}A | | UAAUCC | |
| | | | | _{M}C_{F}U_{M}A_{F}C_{M}G_{F}G | | UU | | | _{M}G_{F}U_{M}A_{F}A_{M}U_{F} | | GAGUU | |
| | | | | _{M}U_{F}^{S}U_{M}^{S}U_{F}- | | | | | C_{M}C_{F}G_{M}A_{F}G^{S} | | | |
| | | | | C7OH-[_{D}Tx-01- | | | | | U_{M}^{S}U_{M}-OH-3′ | | | |
| | | | | 08] | | | | | | | | |
| DT- | 654 | 674 | DTS- | 5′-HO- | 847 | CGGUGU | 1026 | DTS- | 5′-VP- | 967 | AAGAUC | 1118 |
| 001842 | | | 002014 | C_{F}^{S}G_{M}^{S}G_{F}U_{M}G_{F}U | | CAUCUA | | 002874 | A_{M}^{S}A_{F}^{S}G_{M}A_{F}U_{M} | | ACAUAG | |
| | | | | _{M}C_{F}A_{M}U_{F}C_{M}U_{F}A | | UGUGAU | | | C_{F}A_{M}C_{F}A_{M}U_{F}A | | AUGACA | |
| | | | | _{M}U_{F}G_{M}U_{F}G_{M}A_{F}U | | CUU | | | _{M}G_{F}A_{M}U_{F}G_{M}A_{F} | | CCGCU | |
| | | | | _{M}C_{F}^{S}U_{M}^{S}U_{F}- | | | | | C_{M}A_{F}C_{M}C_{F}G_{M}^{S}C | | | |
| | | | | C7OH-_{D}Tx-01-08 | | | | | _{M}^{S}U_{M}OH-3′ | | | |
| DT- | 654 | 674 | DTS- | 5′-HO- | 871 | CGGUGU | 1026 | DTS- | 5′-VP- | 968 | AAGAUC | 1118 |
| 001843 | | | 002875 | C_{F}^{S}G_{M}^{S}G_{M}U_{M}G_{M} | | CAUCUA | | 002876 | A_{M}^{S}A_{F}^{S}G_{M}A_{M}U | | ACAUAG | |
| | | | | U_{M}C_{F}A_{M}U_{F}C_{F}U_{F}A | | UGUGAU | | | _{M}C_{F}A_{M}C_{M}A_{M}U_{M} | | AUGACA | |
| | | | | _{M}U_{M}G_{M}U_{M}G_{M}A_{M} | | CUU | | | A_{M}G_{M}A_{M}U_{F}G_{M} | | CCGCU | |
| | | | | U_{M}C_{M}^{S}U_{M}^{S}U_{M}- | | | | | A_{F}C_{M}A_{M}C_{M}C_{M}G | | | |
| | | | | C7OH-_{D}Tx-01-08 | | | | | _{M}^{S}C_{M}^{S}U_{M}OH-3′ | | | |
| DT- | 211 | 231 | DTS- | 5′-HO- | 872 | CUCCUCC | 1035 | DTS- | 5′-VP- | 969 | AAUACU | 1121 |
| 001844 | | | 002877 | C_{F}^{S}U_{M}^{S}C_{F}C_{M}U_{F}C | | UGUUGC | | 002878 | A_{M}^{S}A_{F}^{S}U_{M}A_{F}C_{M} | | CAGCAAC | |
| | | | | _{M}C_{F}U_{M}G_{F}U_{M}U_{F}G | | UGAGUA | | | U_{F}C_{M}A_{F}G_{M}C_{F}A | | AGGAGG | |
| | | | | _{M}C_{F}U_{M}G_{F}A_{M}G_{F}U | | UU | | | _{M}A_{F}C_{M}A_{F}G_{M}G_{F} | | AGCA | |
| | | | | _{M}A_{F}^{S}U_{M}^{S}U_{F}- | | | | | A_{M}G_{F}G_{M}A_{F}G_{M}^{S} | | | |
| | | | | C7OH-_{D}Tx-01-08 | | | | | C_{M}^{S}A_{M}OH-3′ | | | |
| DT- | 211 | 231 | DTS- | 5′-HO- | 873 | CUCCUCC | 1035 | DTS- | 5′-VP- | 970 | AAUACU | 1121 |
| 001845 | | | 002879 | C_{M}^{S}U_{M}^{S}C_{M}C_{M}U_{M} | | UGUUGC | | 002880 | A_{M}^{S}A_{F}^{S}U_{M}A_{M}C | | CAGCAAC | |
| | | | | C_{M}C_{F}U_{M}G_{F}U_{F}U_{F}G | | UGAGUA | | | _{M}U_{F}C_{M}A_{M}G_{M}C_{M} | | AGGAGG | |
| | | | | _{M}C_{M}U_{M}G_{M}A_{M}G_{M} | | UU | | | A_{M}A_{M}C_{M}A_{F}G_{M} | | AGCA | |
| | | | | U_{M}A_{M}^{S}U_{M}^{S}U_{M}- | | | | | G_{F}A_{M}G_{M}G_{M}A_{M} | | | |
| | | | | C7OH-_{D}Tx-01-08 | | | | | G_{M}^{S}C_{M}^{S}A_{M}OH- | | | |
| | | | | | | | | | 3′ | | | |
| DT- | 214 | 234 | DTS- | 5′-HO- | 874 | CUCCUGU | 1038 | DTS- | 5′-VP- | 971 | AAUGAU | 1123 |
| 001846 | | | 002881 | C_{F}^{S}U_{M}^{S}C_{F}C_{M}U_{F}G | | UGCUGA | | 002882 | A_{M}^{S}A_{F}^{S}U_{M}G_{F}A_{M} | | ACUCAGC | |
| | | | | _{M}U_{F}U_{M}G_{F}C_{M}U_{F}G | | GUAUCA | | | U_{F}A_{M}C_{F}U_{M}C_{F}A | | AACAGG | |
| | | | | _{M}A_{F}G_{M}U_{F}A_{M}U_{F}C | | UU | | | _{M}G_{F}C_{M}A_{F}A_{M}C_{F} | | AGGA | |
| | | | | _{M}A_{F}^{S}U_{M}^{S}U_{F}- | | | | | A_{M}G_{F}G_{M}A_{F}G_{M} | | | |
| | | | | C7OH-_{D}Tx-01-08 | | | | | G_{M}^{S}A_{M}OH-3′ | | | |
| DT- | 214 | 234 | DTS- | 5′-HO- | 875 | CUCCUGU | 1038 | DTS- | 5′-VP- | 972 | AAUGAU | 1123 |
| 001847 | | | 002883 | C_{M}^{S}U_{M}^{S}C_{M}C_{M}U_{M} | | UGCUGA | | 002884 | A_{M}^{S}A_{F}^{S}U_{M}G_{M}A | | ACUCAGC | |
| | | | | G_{M}U_{F}U_{M}G_{F}C_{F}U_{F} | | GUAUCA | | | _{M}U_{F}A_{M}C_{M}U_{M}C_{M} | | AACAGG | |
| | | | | G_{M}A_{M}G_{M}U_{M}A_{M}U | | UU | | | A_{M}G_{M}C_{M}A_{F}A_{M}C | | AGGA | |
| | | | | _{M}C_{M}A_{M}^{S}U_{M}^{S}U_{M}- | | | | | _{F}A_{M}G_{M}G_{M}A_{M}G_{M} | | | |
| | | | | C7OH-_{D}Tx-01-08 | | | | | ^{S}G_{M}^{S}A_{M}OH-3′ | | | |
| DT- | 225 | 245 | DTS- | 5′-HO- | 812 | GAGUAU | 1054 | DTS- | 5′-VP- | 973 | ACGUGG | 1126 |
| 001848 | | | 001965 | G_{F}^{S}A_{M}^{S}G_{F}U_{M}A_{F}U | | CAUCGUC | | 002885 | A_{M}^{S}C_{F}^{S}G_{M}U_{F}G_{M} | | AGGACG | |
| | | | | _{M}C_{F}A_{M}U_{F}C_{M}G_{F}U | | CUCCACG | | | G_{F}A_{M}G_{F}G_{M}A_{F}C | | AUGAUA | |
| | | | | _{M}C_{F}C_{M}U_{F}C_{M}C_{F}A_{M} | | U | | | _{M}G_{F}A_{M}U_{F}G_{M}A_{F} | | CUCAG | |
| | | | | C_{F}^{S}G_{M}^{S}U_{F}-C7OH- | | | | | U_{M}A_{F}C_{M}U_{F}C_{M} | | | |
| | | | | _{D}Tx-01-08 | | | | | A_{M}^{S}G_{M}OH-3′ | | | |
| DT- | 225 | 245 | DTS- | 5′-HO- | 876 | GAGUAU | 1054 | DTS- | 5′-VP- | 974 | ACGUGG | 1126 |
| 001849 | | | 002886 | G_{M}^{S}A_{M}^{S}G_{M}U_{M}A_{M} | | CAUCGUC | | 002887 | A_{M}^{S}C_{F}^{S}G_{M}U_{M}G | | AGGACG | |
| | | | | U_{M}C_{F}A_{M}U_{F}C_{F}G_{F}U | | CUCCACG | | | _{M}G_{F}A_{M}G_{M}G_{M}A_{M} | | AUGAUA | |
| | | | | _{M}C_{M}C_{M}U_{M}C_{M}C_{M}A | | U | | | C_{M}G_{M}A_{M}U_{F}G_{M} | | CUCAG | |
| | | | | _{M}C_{M}^{S}G_{M}^{S}U_{M}- | | | | | A_{F}U_{M}A_{M}C_{M}U_{M}C | | | |
| | | | | C7OH-_{D}Tx-01-08 | | | | | _{M}^{S}A_{M}^{S}G_{M}OH-3′ | | | |
| DT- | 213 | 233 | DTS- | 5′-HO- | 877 | CCUCCUG | 1018 | DTS- | 5′-VP- | 902 | AUGAUA | 1144 |
| 001858 | | | 002898 | C_{M}^{S}C_{M}^{S}U_{M}C_{M}C_{M} | | UUGCUG | | 001897 | A_{M}^{S}U_{F}^{S}G_{M}A_{M}U | | CUCAGCA | |
| | | | | U_{M}G_{F}U_{M}U_{F}G_{F}C_{F} | | AGUAUC | | | _{M}A_{F}C_{M}U_{M}C_{M}A_{M} | | ACAGGA | |
| | | | | U_{M}G_{M}A_{M}G_{M}U_{M}A | | AU | | | G_{M}C_{M}A_{M}A_{F}C_{M}A | | GGAG | |
| | | | | _{M}U_{M}C_{M}A_{M}^{S}U_{M}- | | | | | G_{M}G_{M}A_{M}G_{M}G_{M} | | | |
| | | | | C7OH-[_{D}Tx-01- | | | | | ^{S}A_{M}^{S}G_{M}-OH-3′ | | | |
| | | | | 08] | | | | | | | | |
| DT- | 213 | 233 | DTS- | 5′-HO- | 878 | CCUCCUG | 1018 | DTS- | 5′-VP- | 902 | AUGAUA | 1144 |
| 001859 | | | 002899 | C_{M}^{S}C_{M}^{S}U_{F}C_{M}C_{M}U | | UUGCUG | | 001897 | A_{M}^{S}U_{F}^{S}G_{M}A_{M}U | | CUCAGCA | |
| | | | | _{M}G_{F}U_{M}U_{F}G_{F}C_{F}U_{M} | | AGUAUC | | | _{M}A_{F}C_{M}U_{M}C_{M}A_{M} | | ACAGGA | |
| | | | | G_{M}A_{M}G_{M}U_{M}A_{M}U | | AU | | | G_{M}C_{M}A_{M}A_{F}C_{M}A | | GGAG | |
| | | | | _{M}C_{M}^{S}A_{M}^{S}U_{M}- | | | | | G_{M}G_{M}A_{M}G_{M}G_{M} | | | |
| | | | | C7OH-[_{D}Tx-01- | | | | | ^{S}A_{M}^{S}G_{M}-OH-3′ | | | |
| | | | | 08] | | | | | | | | |
| DT- | 213 | 233 | DTS- | 5′-HO- | 774 | CCUCCUG | 1018 | DTS- | 5′-VP- | 975 | AUGAUA | 1144 |
| 001860 | | | 001896 | C_{M}^{S}C_{M}^{S}U_{M}C_{M}C_{M} | | UUGCUG | | 002900 | A_{M}^{S}U_{F}^{S}G_{M}A_{M}U | | CUCAGCA | |
| | | | | U_{M}G_{F}U_{M}U_{F}G_{F}C_{F} | | AGUAUC | | | _{M}A_{F}C_{M}U_{M}C_{M}A_{M} | | ACAGGA | |
| | | | | U_{M}G_{M}A_{M}G_{M}U_{M}A | | AU | | | G_{M}C_{M}A_{M}A_{F}C_{M}A | | GGAG | |
| | | | | _{M}U_{M}C_{M}^{S}A_{M}^{S}U_{M}- | | | | | G_{M}G_{M}A_{M}G_{M}G_{M} | | | |
| | | | | C7OH- | | | | | ^{S}A_{M}^{S}G_{E}OH-3′ | | | |
| | | | | _{D}Tx-01-08 | | | | | | | | |

### Example 5: In Vitro Testing of Unconjugated siRNAs Targeting PMP22

Unconjugated compounds were tested for their ability to inhibit the expression of PMP22 in human Schwann cells that express endogenous PMP22 and HEK cells engineered to express human PMP22 (HEK-PMP22 cells). Transfection experiments and PMP22 quantitation were performed according to the methods described herein.

Schwann cells and HEK-PMP22 cells were transfected with siRNAs at doses of 0.3 nM, 3 nM, and 30 nM. RNA was isolated 48 hours later, reverse transcribed to cDNA and PMP22 expression was quantified by qPCR. The average PMP22 expression for each of four replicates was calculated and shown in Tables 5 through 10. Several of the siRNAs inhibited PMP22 expression in a dose-dependent manner.

TABLE 5
Transfection of PMP22 siRNAs into human Schwann cells

| | PMP22 mRNA % Remaining | | | | | |
|---|---|---|---|---|---|---|
| | 0.3 nM | | 3 nM | | 30 nM | |
| Treatment | Mean | S.E.M. | Mean | S.E.M. | Mean | S.E.M. |
| DT-000390 | 46.8 | 4.4 | 36.1 | 1.6 | 36.1 | 2.9 |
| DT-000391 | 67.3 | 3.3 | 72.9 | 4.2 | 64.3 | 4.9 |
| DT-000392 | 28.8 | 1.9 | 24.0 | 0.4 | 21.1 | 2.1 |
| DT-000393 | 97.4 | 4.4 | 102.6 | 7.6 | 105.4 | 11.0 |
| DT-000394 | 37.7 | 1.3 | 14.7 | 4.5 | 13.8 | 1.7 |
| DT-000395 | 35.0 | 3.2 | 14.0 | 1.2 | 20.7 | 2.3 |
| DT-000396 | 27.2 | 1.0 | 16.0 | 2.7 | 14.4 | 1.3 |
| DT-000397 | 37.5 | 2.8 | 12.6 | 1.1 | 8.9 | 1.1 |
| DT-000398 | 19.5 | 1.6 | 9.2 | 1.0 | 5.1 | 0.16 |
| DT-000399 | 80.3 | 1.1 | 45.3 | 2.3 | 34.2 | 6.0 |
| DT-000400 | 77.2 | 6.1 | 39.4 | 3.4 | 51.0 | 4.5 |
| DT-000401 | 86.9 | 5.4 | 114.4 | 23.2 | 86.4 | 4.9 |

TABLE 6
Transfection of PMP22 siRNAs into HEK-PMP22 cells

| | PMP22 mRNA % Remaining | | | | | |
|---|---|---|---|---|---|---|
| | 0.3 nM | | 3 nM | | 30 nM | |
| Treatment | Mean | S.E.M. | Mean | S.E.M. | Mean | S.E.M. |
| DT-000390 | 112.5 | 12.4 | 86.5 | 2.6 | 54.1 | 1.4 |
| DT-000391 | 99.3 | 6.7 | 106.7 | 5.2 | 94.8 | 0.8 |
| DT-000392 | 107 | 11.4 | 75 | 4.4 | 39.2 | 1.1 |
| DT-000393 | 104.7 | 7.9 | 104.4 | 2.3 | 123.8 | 2.1 |
| DT-000394 | 109 | 7.4 | 72.7 | 3.4 | 24.4 | 1.1 |
| DT-000395 | 97.0 | 2.3 | 86.0 | 1.1 | 47.2 | 2.2 |
| DT-000396 | 89.9 | 1.7 | 48.6 | 2.9 | 18.3 | 1.4 |
| DT-000397 | 85.6 | 3.0 | 52.8 | 4.1 | 22.4 | 1.9 |
| DT-000398 | 83.3 | 2.6 | 39.3 | 2.4 | 19.1 | 1.7 |
| DT-000399 | 94.9 | 2.2 | 84.0 | 8.0 | 65.5 | 11.6 |
| DT-000400 | 99.0 | 3.3 | 77.1 | 6.7 | 39.6 | 6.7 |
| DT-000401 | 104.4 | 5.7 | 112.7 | 9.9 | 97.3 | 5.3 |

TABLE 7
Transfection of PMP22 siRNAs into human Schwann cells

| | PMP22 mRNA % Remaining | | | | | |
|---|---|---|---|---|---|---|
| | 0.3 nM | | 3 nM | | 30 nM | |
| Treatment | Mean | S.E.M. | Mean | S.E.M. | Mean | S.E.M. |
| DT-000402 | 100.4 | 11.0 | 44.9 | 2.8 | 36.8 | 2.0 |
| DT-000403 | 103.6 | 4.0 | 90.9 | 4.7 | 81.2 | 12.0 |
| DT-000404 | 127.7 | 21.1 | 92.1 | 3.0 | 85.0 | 2.8 |
| DT-000405 | 100.7 | 15.0 | 20.3 | 3.7 | 26.6 | 7.3 |
| DT-000406 | 93.5 | 6.6 | 71.0 | 14.6 | 50.5 | 6.1 |
| DT-000407 | 117.3 | 8.1 | 90.0 | 1.9 | 104.8 | 9.3 |
| DT-000408 | 99.9 | 3.2 | 113.6 | 21.5 | 94.3 | 16.8 |
| DT-000409 | 109.6 | 12.3 | 82.1 | 1.3 | 71.8 | 2.2 |
| DT-000410 | 39.5 | 10.1 | 19.2 | 10.0 | 4.2 | 0.9 |
| DT-000411 | 83.5 | 1.5 | 46.1 | 2.7 | 37.8 | 1.7 |
| DT-000412 | 77.1 | 1.0 | 33.5 | 3.0 | 25.3 | 4.2 |
| DT-000413 | 70.7 | 1.0 | 38.7 | 3.6 | 39.5 | 2.9 |

TABLE 8
Transfection of PMP22 siRNAs into HEK-PMP22 Cells

| | PMP22 mRNA % Remaining | | | | | |
|---|---|---|---|---|---|---|
| | 0.3 nM | | 3 nM | | 30 nM | |
| Treatment | Mean | S.E.M. | Mean | S.E.M. | Mean | S.E.M. |
| DT-000402 | 93.8 | 2.4 | 77.9 | 1.8 | 56.0 | 1.2 |
| DT-000403 | 93.8 | 3.1 | 88.2 | 1.7 | 74.2 | 1.2 |
| DT-000404 | 99.1 | 2.1 | 102.3 | 2.4 | 96.7 | 1.2 |
| DT-000405 | 84.5 | 2.1 | 43.8 | 2.6 | 27.6 | 2.2 |
| DT-000406 | 96.1 | 8.3 | 61.8 | 1.1 | 40.8 | 2.0 |
| DT-000407 | 94.7 | 1.6 | 105.2 | 9.4 | 93.7 | 2.7 |
| DT-000408 | 105.7 | 1.4 | 103.5 | 2.5 | 118.2 | 6.7 |
| DT-000409 | 117.5 | 22 | 88.1 | 1.9 | 87.1 | 6.8 |
| DT-000410 | 37.0 | 3.4 | 19.2 | 0.6 | 9.4 | 0.8 |
| DT-000411 | 114.3 | 10.0 | 45.4 | 3.0 | 28.6 | 0.6 |
| DT-000412 | 83.3 | 4.5 | 45.0 | 2.7 | 27.1 | 0.9 |
| DT-000413 | 86.0 | 3.5 | 47.7 | 1.6 | 42.4 | 5.3 |

TABLE 9
Transfection of PMP22 siRNAs into Human Schwann Cells

| | PMP22 mRNA % Remaining | | | | | |
|---|---|---|---|---|---|---|
| | 0.3 nM | | 3 nM | | 30 nM | |
| Treatment | Mean | S.E.M. | Mean | S.E.M. | Mean | S.E.M. |
| DT-000410 | 40.2 | 1.9 | 13.8 | 0.2 | 13.0 | 3.2 |
| DT-000414 | 45.1 | 2.1 | 10.7 | 0.9 | 4.9 | 0.3 |
| DT-000415 | 85.8 | 1.6 | 34.6 | 2.5 | 20.2 | 1.5 |
| DT-000416 | 86.5 | 0.6 | 78.9 | 3.0 | 70.1 | 3.5 |
| DT-000417 | 105.5 | 8.9 | 85.3 | 2.2 | 74.2 | 5.0 |
| DT-000418 | 89.7 | 2.3 | 17.4 | 1.3 | 7.2 | 0.7 |
| DT-000419 | 102.7 | 3.6 | 94.7 | 6.4 | 70.5 | 4.3 |
| DT-000420 | 60.7 | 2.4 | 14.9 | 1.3 | 7.7 | 0.6 |
| DT-000421 | 65.3 | 3.5 | 15.4 | 1.0 | 8.5 | 1.3 |
| DT-000422 | 69.5 | 1.1 | 32.8 | 2.6 | 20.1 | 0.9 |
| DT-000423 | 121.2 | 6.4 | 101.4 | 6.1 | 79.1 | 4.3 |
| DT-000424 | 117.9 | 5.6 | 52.8 | 3.6 | 40.1 | 1.2 |
| DT-000425 | 67.2 | 7.1 | 18.0 | 1.0 | 8.2 | 0.8 |

TABLE 10
Transfection of PMP22 siRNAs into HEK-PMP22 Cells

| | PMP22 mRNA % Remaining | | | | | |
|---|---|---|---|---|---|---|
| | 0.3 nM | | 3 nM | | 30 nM | |
| Treatment | Mean | S.E.M. | Mean | S.E.M. | Mean | S.E.M. |
| DT-000414 | 62.1 | 5.0 | 8.0 | 2.1 | 4.3 | 0.9 |
| DT-000415 | 79.8 | 6.1 | 18.5 | 2.4 | 4.8 | 1.6 |
| DT-000416 | 84.6 | 2.5 | 62.4 | 3.1 | 41.0 | 6.5 |
| DT-000417 | 82.9 | 5.7 | 67.2 | 4.3 | 46.9 | 6.8 |
| DT-000418 | 93.2 | 7.3 | 23.0 | 10.8 | 13.6 | 6.1 |
| DT-000419 | 94.1 | 5.1 | 71.1 | 7.3 | 42.7 | 6.7 |
| DT-000420 | 82.8 | 1.8 | 20.1 | 2.0 | 8.6 | 0.4 |
| DT-000421 | 84.4 | 2.5 | 28.5 | 1.4 | 13.7 | 1.1 |
| DT-000422 | 91.6 | 2.4 | 57.5 | 3.0 | 18.6 | 1.2 |
| DT-000423 | 87.4 | 1.8 | 83.0 | 2.3 | 63.5 | 3.3 |
| DT-000424 | 97.3 | 4.4 | 69.1 | 2.7 | 35.9 | 1.2 |
| DT-000425 | 92.1 | 2.6 | 39.5 | 2.7 | 15.8 | 0.8 |

Schwann cells and HEK-PMP22 cells were transfected with siRNAs at doses of 3 nM and 30 nM. RNA was isolated 48 hours later, reverse transcribed to cDNA and PMP22 expression was quantified by qPCR. The average PMP22 expression for each of four replicates was calculated and shown in Tables 11 and 12. Several of the siRNAs inhibited PMP22 expression in a dose-dependent manner.

TABLE 11
Transfection of PMP22 siRNAs into
HEK-PMP22 and Schwann Cells

| | PMP22 mRNA % Remaining | | | | | | | |
|---|---|---|---|---|---|---|---|---|
| | HEK PMP22 | | | | Schwann Cells | | | |
| | 3 nM | | 30 nM | | 3 nM | | 30 nM | |
| | Mean | | Mean | | Mean | | Mean | |
| Treatment | (SEM) | | (SEM) | | (SEM) | | (SEM) | |
| DT-000390 | 110.1 | (2.9) | 96.3 | (4.3) | | | | |
| DT-000414 | 37 | (2.3) | 11.2 | (1.1) | 16.9 | (1) | 4.4 | (1.1) |
| DT-000845 | 61.4 | (2.4) | 16.9 | (0.7) | 25.6 | (3.4) | 4.7 | (0.8) |
| DT-000846 | 59.5 | (4.4) | 23.2 | (1.8) | 22.7 | (1.1) | 6.2 | (1.2) |
| DT-000847 | 74.3 | (1.1) | 36.5 | (6.7) | 102.5 | (9.2) | 46.7 | (5.7) |
| DT-000848 | 106.1 | (1.4) | 78.5 | (5.6) | 56 | (8.4) | 14 | (2) |
| DT-000849 | 57.2 | (4.3) | 28.8 | (6.7) | 17 | (0.4) | 3.9 | (0.4) |
| DT-000850 | 77.2 | (8.5) | 41.9 | (11.9) | 20.9 | (1.2) | 4.9 | (0.3) |
| DT-000851 | 103.5 | (2.1) | 77.3 | (8.3) | 48.2 | (4.2) | 24.1 | (1.8) |
| DT-000852 | 92.7 | (1.8) | 48.4 | (7.9) | 25 | (2.9) | 6.9 | (2) |
| DT-000853 | 72.5 | (4.6) | 37 | (9.4) | 20.4 | (0.8) | 5.8 | (0.5) |
| DT-000854 | 81.6 | (2.7) | 56.5 | (1.8) | 67.5 | (0.5) | 32.2 | (3.9) |
| DT-000855 | 61.7 | (4.3) | 35.9 | (1.7) | 18.8 | (1.3) | 3.9 | (0.5) |
| DT-000856 | 84.4 | (2.8) | 70.5 | (2.5) | 24.8 | (0.8) | 7 | (0.5) |
| DT-000857 | 91.4 | (2.4) | 84.6 | (1) | 55.8 | (1.7) | 22.5 | (2.2) |
| DT-000858 | 66 | (5.3) | 45.9 | (3.6) | 22.7 | (0.3) | 7.1 | (0.1) |
| DT-000859 | 89.4 | (2.4) | 71.6 | (5.5) | 48.1 | (1.3) | 22 | (0.5) |
| DT-000860 | 101.7 | (2) | 94.2 | (5) | 90.3 | (4.6) | 68.8 | (3.2) |
| DT-000861 | 95.1 | (1.6) | 87.3 | (3.8) | 47.8 | (4.4) | 33.1 | (5) |
| DT-000862 | 92 | (2.1) | 55.8 | (3.1) | 78.3 | (6.1) | 58.6 | (2.2) |
| DT-000863 | 95.5 | (1.7) | 80.5 | (3.1) | 54.7 | (6) | 35.3 | (1.6) |
| DT-000864 | 99.6 | (1.3) | 92.7 | (3.1) | 97.5 | (9) | 65.9 | (1.1) |
| DT-000865 | 71.2 | (6.6) | 35.5 | (4.8) | 27.7 | (4.8) | 8.6 | (3.2) |
| DT-000866 | 100.7 | (9) | 68.7 | (5.1) | 39.5 | (1.2) | 19.6 | (1.5) |
| DT-000867 | 100.5 | (1.6) | 85.2 | (2.5) | 91.3 | (3.6) | 36.4 | (1.7) |
| DT-000868 | 92.4 | (3.7) | 66.6 | (6.6) | 48 | (5.3) | 20.6 | (2.5) |
| DT-000869 | 86.6 | (6.2) | 50.1 | (6) | 41.4 | (1.7) | 17.5 | (0.5) |
| DT-000870 | 95.8 | (0.9) | 73.3 | (4.2) | 54.2 | (1.9) | 40.2 | (1.8) |
| DT-000871 | 91.6 | (3.9) | 69.4 | (5) | 61.8 | (6.5) | 34.6 | (1) |
| DT-000872 | 85.2 | (4.4) | 54 | (5.3) | 47.3 | (3.9) | 14.8 | (0.3) |
| DT-000873 | 39.2 | (5.4) | 11.9 | (1.8) | 11.7 | (0.5) | 3.4 | (0.2) |
| DT-000874 | 100.3 | (1.6) | 99.3 | (1.8) | 91.5 | (1) | 79.5 | (3) |
| DT-000875 | 67.9 | (2.7) | 33.4 | (4.3) | 30 | (1.1) | 14.2 | (0.5) |
| DT-000876 | 66.5 | (3.7) | 32.5 | (5.8) | 31.3 | (0.8) | 8.8 | (0.2) |
| DT-000877 | 87.9 | (3.1) | 56.8 | (6.6) | 30.3 | (6.2) | 13.9 | (2.8) |

| | | | | | | | |
|---|---|---|---|---|---|---|---|
| DT-000878 | 95.4 | (3.6) | 97.1 | (0.6) | 112 | 25.2 | (9.3) |
| | | | | | (23.7) | | |

TABLE 12
Transfection of PMP22 siRNAs into
HEK-PMP22 Cells and Schwann Cells

| | PMP22 mRNA % Remaining | | | | | | | |
|---|---|---|---|---|---|---|---|---|
| | HEK PMP22 | | | | Schwann Cells | | | |
| | 3 nM | | 30 nM | | 3 nM | | 30 nM | |
| | Mean | | Mean | | Mean | | Mean | |
| Treatment | (SEM) | | (SEM) | | (SEM) | | (SEM) | |
| DT-000414 | 4.7 | (0.3) | 4.4 | (0.6) | 13.2 | (0.8) | 4.9 | (0.3) |
| DT-000879 | 22.9 | (0.6) | 37.3 | (0.8) | 74 | (3.7) | 76.3 | (7.5) |
| DT-000880 | 5.4 | (0.2) | 4.9 | (0.4) | 34.5 | (1.6) | 25 | (0.8) |
| DT-000881 | 9.3 | (0.6) | 7.3 | (0.5) | 55.7 | (6.7) | 31.4 | (1.6) |
| DT-000882 | 4.2 | (0.2) | 6.4 | (0.5) | 13.3 | (0.7) | 14 | (6.5) |
| DT-000883 | 10.1 | (1) | 12.9 | (1) | 51 | (1.6) | 32.8 | (1.6) |
| DT-000884 | 7.3 | (0.8) | 10.9 | (0.9) | 26.3 | (2) | 13.6 | (0.7) |
| DT-000885 | 12.5 | (0.3) | 17.9 | (0.4) | 61.8 | (3.9) | 23.4 | (3) |
| DT-000886 | 7.1 | (0.2) | 5.9 | (0.3) | 57.2 | (1.8) | 34.6 | (1.9) |
| DT-000887 | 10.6 | (1) | 8.8 | (0.5) | 38.8 | (1.8) | 20.4 | (1.6) |
| DT-000888 | 73.8 | (3.9) | 90 | (3.2) | 92.6 | (6.5) | 82.8 | (6.8) |
| DT-000889 | 52.2 | (2.3) | 50.6 | (2.6) | 107.4 | (8.9) | 84.9 | (8.7) |
| DT-000890 | 109.2 | (3.5) | 107.1 | (0.9) | 101.8 | (1.8) | 80.6 | (3.4) |
| DT-000891 | 30.3 | (2.3) | 23.6 | (4) | 74.9 | (2.5) | 68.6 | (7.4) |
| DT-000892 | 12.7 | (0.9) | 8 | (0.2) | 52.7 | (1.8) | 38.9 | (2.8) |
| DT-000893 | 69.3 | (1.5) | 89.1 | (9.9) | 121.8 | (6.7) | 92.5 | (4.5) |
| DT-000894 | 13.6 | (0.3) | 15 | (0.9) | 64 | (5.8) | 62.2 | (3.8) |
| DT-000895 | 8.6 | (0.6) | 6.8 | (0.7) | 72.4 | (11.2) | 35.1 | (1.8) |
| DT-000896 | 17 | (0.3) | 10.7 | (0.7) | 89.6 | (3.7) | 53.9 | (5.6) |
| DT-000897 | 6.1 | (0.3) | 5.9 | (0.7) | 44.5 | (2.2) | 30.7 | (0.9) |
| DT-000898 | 120.2 | (7.2) | 99.9 | (3.7) | 122.5 | (16) | 95.8 | (2.1) |
| DT-000899 | 34.8 | (3.1) | 16.3 | (0.3) | 45.7 | (2.8) | 26.2 | (2.1) |
| DT-000900 | 4 | (0.4) | 3.6 | (0.3) | 17.3 | (0.9) | 11.5 | (0.4) |
| DT-000901 | 4.6 | (0.4) | 3.7 | (0.4) | 8.3 | (2.1) | 5.3 | (0.3) |
| DT-000902 | 13.1 | (0.7) | 11 | (0.5) | 27.8 | (1) | 22.2 | (2.3) |

Compounds DT-000904 through DT-000928 target the 3′-UTR of human PMP22. As HEK-PMP22 cells do not express the 3′-UTR of PMP22, these compounds were tested in Schwann cells only.

TABLE 13
Transfection of siRNAs into Schwann Cells

| PMP22 mRNA % Remaining | |
|---|---|
| 3 nM | 30 nM |

| Treatment | Mean | S.E.M. | Mean | S.E.M. |
|---|---|---|---|---|
| DT-000414 | 2.9 | 0.3 | 1.5 | 0.1 |
| DT-000904 | 9.9 | 0.7 | 3.6 | 0.4 |
| DT-000905 | 7.0 | 0.5 | 4.4 | 0.7 |
| DT-000906 | 4.0 | 0.2 | 2.6 | 0.5 |
| DT-000907 | 5.8 | 0.5 | 4.5 | 0.7 |
| DT-000908 | 1.5 | 0.1 | 0.7 | 0.1 |
| DT-000909 | 3.6 | 0.6 | 1.9 | 0.2 |
| DT-000910 | 114.2 | 7.7 | 78.6 | 2.1 |
| DT-000911 | 5.0 | 0.5 | 3.7 | 0.8 |
| DT-000912 | 3.8 | 0.3 | 3.6 | 0.2 |
| DT-000913 | 9.1 | 1.0 | 9.8 | 2.8 |
| DT-000914 | 4.2 | 1.0 | 2.2 | 0.2 |
| DT-000915 | 5.1 | 0.8 | 4.2 | 0.7 |
| DT-000916 | 9.2 | 1.6 | 4.4 | 0.7 |
| DT-000917 | 5.2 | 0.7 | 4.8 | 0.5 |
| DT-000918 | 31.5 | 2.4 | 19.9 | 2.0 |
| DT-000919 | 9.8 | 0.8 | 6.0 | 0.7 |
| DT-000920 | 13.9 | 1.6 | 7.3 | 0.4 |
| DT-000921 | 85.8 | 10.2 | 82.9 | 2.0 |
| DT-000922 | 22.0 | 2.3 | 16.6 | 1.9 |
| DT-000923 | 5.7 | 0.8 | 3.1 | 0.5 |
| DT-000924 | 23.2 | 2.1 | 16.2 | 1.7 |
| DT-000925 | 4.4 | 0.6 | 3.6 | 0.4 |
| DT-000926 | 24.9 | 3.8 | 18.5 | 0.6 |
| DT-000927 | 7.1 | 0.3 | 6.0 | 0.7 |
| DT-000928 | 6.6 | 0.5 | 6.9 | 0.8 |

Compounds DT-001010 through DT-001034 target the 5′-UTR of human PMP22. As HEK-PMP22 cells do not express the 5′-UTR of PMP22, these compounds were tested in Schwann cells only.

TABLE 14
Transfection of siRNAs into Schwann Cells

| PMP22 mRNA % Remaining | |
|---|---|
| 3 nM | 30 nM |

| Treatment | Mean | S.E.M. | Mean | S.E.M. |
|---|---|---|---|---|
| DT-000414 | 3.9 | 0.4 | 1.9 | 0.4 |
| DT-001010 | 122.1 | 27.1 | 88.8 | 7.9 |
| DT-001011 | 94.3 | 3.5 | 76.7 | 2.6 |
| DT-001012 | 98.1 | 7.0 | 80.8 | 1.5 |
| DT-001013 | 87.0 | 11.5 | 77.0 | 7.8 |
| DT-001014 | 112.8 | 21.5 | 74.0 | 1.9 |
| DT-001015 | 93.2 | 10.0 | 75.0 | 1.7 |
| DT-001016 | 109.3 | 18.5 | 79.5 | 7.0 |
| DT-001017 | 89.3 | 4.6 | 82.2 | 7.3 |
| DT-001018 | 92.5 | 12.3 | 63.0 | 2.0 |
| DT-001019 | 66.5 | 15.3 | 51.6 | 7.5 |
| DT-001020 | 96.8 | 1.5 | 86.1 | 9.5 |
| DT-001021 | 96.2 | 3.5 | 89.7 | 1.6 |
| DT-001022 | 98.9 | 4.7 | 95.6 | 1.4 |
| DT-001023 | 93.3 | 4.7 | 84.4 | 4.6 |
| DT-001024 | 79.2 | 4.6 | 74.4 | 2.0 |
| DT-001025 | 91.8 | 2.1 | 90.3 | 10.4 |
| DT-001026 | 102.6 | 2.3 | 86.1 | 1.1 |
| DT-001027 | 88.0 | 1.2 | 81.1 | 1.6 |
| DT-001028 | 63.8 | 1.3 | 57.3 | 2.0 |
| DT-001029 | 83.9 | 1.2 | 69.8 | 3.3 |
| DT-001030 | 17.0 | 1.4 | 8.8 | 0.6 |
| DT-001031 | 12.6 | 1.5 | 7.5 | 0.5 |
| DT-001032 | 39.6 | 2.5 | 36.6 | 4.1 |
| DT-001033 | 63.9 | 1.7 | 82.9 | 4.3 |
| DT-001034 | 67.7 | 1.5 | 67.0 | 4.9 |

Certain compounds were selected for additional testing in a dose-response experiment. Schwann cells and HEK-PMP22 cells were transfected with siRNAs at doses of 0.3 nM, 1 nM, 3 nM, 10 nM and 30 nM. RNA was isolated 48 hours later, reverse transcribed to cDNA and PMP22 expression was quantified by qPCR. The average PMP22 expression for each of four replicates was calculated and shown in Tables 15 through 18. Several of the siRNAs inhibited PMP22 expression in a dose-dependent manner.

TABLE 15
Transfection of siRNAs into HEK PMP22 Cells: Dose Response

| | PMP22 mRNA % Remaining | | | | | | | | | |
|---|---|---|---|---|---|---|---|---|---|---|
| | 0.3 nM | | 1 nM | | 3 nM | | 10 nM | | 30 nM | |
| Treatment | Mean (SEM) | | Mean (SEM) | | Mean (SEM) | | Mean (SEM) | | Mean (SEM) | |
| DT-000414 | 26.4 | (1.8) | 12.2 | (0.9) | 6.5 | (0.4) | 4.3 | (0.4) | 4.2 | (0.3) |
| DT-000845 | 41.9 | (3.8) | 18.8 | (0.6) | 9 | (0.1) | 6.6 | (0.3) | 5.5 | (0.6) |
| DT-000846 | 48.2 | (2.3) | 22.8 | (0.4) | 9.9 | (1.1) | 6.2 | (0.4) | 6.4 | (0.4) |
| DT-000847 | 51.5 | (1.1) | 25.6 | (1.4) | 11.4 | (0.7) | 7.7 | (0.8) | 6.8 | (0.7) |
| DT-000849 | 64 | (11.5) | 22.4 | (0.9) | 11.9 | (0.7) | 8 | (0.7) | 8.2 | (0.8) |
| DT-000853 | 70.5 | (1.2) | 32 | (1.5) | 17.2 | (0.8) | 12.5 | (1.3) | 10 | (0.5) |
| DT-000865 | 101.1 | (4.1) | 53.9 | (1.6) | 21.8 | (1.6) | 15.3 | (0.7) | 11.7 | (0.6) |
| DT-000873 | 20.6 | (2.6) | 9.5 | (1.3) | 5.2 | (0.7) | 6.3 | (0.7) | 4.6 | (0.7) |
| DT-000875 | 26.3 | (3) | 10 | (1.1) | 5.2 | (1.1) | 3.9 | (0.5) | 4 | (0.2) |
| DT-000876 | 58.7 | (7.7) | 29.5 | (0.9) | 25.6 | (3.3) | 13.9 | (1.1) | 9.5 | (0.6) |

TABLE 16
Transfection of siRNAs into HEK PMP22 Cells: Dose Response

| | PMP22 mRNA % Remaining | | | | | | | | | |
|---|---|---|---|---|---|---|---|---|---|---|
| | 0.3 nM | | 1 nM | | 3 nM | | 10 nM | | 30 nM | |
| Treatment | Mean (SEM) | | Mean (SEM) | | Mean (SEM) | | Mean (SEM) | | Mean (SEM) | |
| DT-000414 | 93.2 | (2) | 89.8 | (2.3) | 75.7 | (2) | 50.6 | (1.4) | 18.6 | (0.8) |
| DT-000879 | 89.6 | (2) | 89.5 | (1.4) | 89 | (1.5) | 84.2 | (2.6) | 48.5 | (1.2) |
| DT-000880 | 97.5 | (1.6) | 87.1 | (1.1) | 78.8 | (1.4) | 47.6 | (1.5) | 24.6 | (4.2) |
| DT-000881 | 95.6 | (4) | 98.5 | (2.6) | 89.4 | (2.3) | 69.4 | (4.6) | 32.5 | (2.3) |
| DT-000882 | 66.1 | (3.1) | 66.5 | (4.1) | 54.3 | (4.6) | 31.2 | (1.7) | 13.4 | (1) |
| DT-000883 | 90.7 | (2.1) | 78.5 | (1.8) | 66.8 | (1.9) | 37.9 | (2.1) | 19.3 | (1.2) |
| DT-000884 | 96 | (4.4) | 90.6 | (4) | 82.8 | (3.8) | 55.4 | (3.8) | 24.2 | (1.5) |
| DT-000885 | 96.8 | (3.5) | 92 | (3.5) | 91.7 | (1.8) | 58.8 | (1.2) | 24.7 | (1.3) |
| DT-000886 | 84.3 | (1.6) | 85.9 | (2) | 87.8 | (5.6) | 78.8 | (1) | 35.7 | (2.1) |
| DT-000887 | 97 | (0.7) | 86.9 | (3) | 88 | (1.8) | 68.8 | (0.5) | 34 | (1.8) |
| DT-000891 | 82.2 | (1.7) | 91.6 | (1.9) | 83.4 | (2) | 68.3 | (0.9) | 37.6 | (1.4) |
| DT-000892 | 87.9 | (2.6) | 89.7 | (0.5) | 87.2 | (2.2) | 63.5 | (1.9) | 30.2 | (2.1) |
| DT-000894 | 84.7 | (1.3) | 86.6 | (0.8) | 83 | (2) | 53.7 | (1.5) | 26.5 | (0.5) |
| DT-000895 | 70.2 | (1.8) | 72.2 | (1.8) | 69.2 | (0.6) | 48.4 | (0.5) | 27.5 | (3.8) |
| DT-000896 | 88.3 | (1.2) | 85.2 | (2.5) | 82.9 | (0.5) | 50.1 | (3.5) | 22.5 | (2.2) |
| DT-000897 | 78.6 | (2.6) | 76.6 | (4.7) | 77.9 | (3.4) | 46.3 | (2.2) | 17.8 | (1.1) |
| DT-000899 | 100 | (2) | 105.8 | (2.3) | 105.4 | (2.7) | 101.6 | (1.6) | 78.3 | (7.6) |
| DT-000900 | 92.7 | (2.7) | 95.8 | (3.1) | 92 | (2.4) | 68.8 | (1.7) | 27.3 | (0.9) |
| DT-000901 | 95.6 | (2.1) | 95.6 | (2.3) | 67.5 | (1.9) | 40.7 | (2.1) | 16.1 | (0.9) |
| DT-000902 | 114.2 | (4.6) | 120.2 | (6.7) | 113.1 | (6.5) | 103.3 | (6.2) | 68.9 | (4.4) |

TABLE 17
Transfection of siRNAs into HEK PMP22 Cells: Dose Response

| | PMP22 mRNA % Remaining | | | | | | | | | |
|---|---|---|---|---|---|---|---|---|---|---|
| | 0.3 nM | | 1 nM | | 3 nM | | 10 nM | | 30 nM | |
| Treatment | Mean (SEM) | | Mean (SEM) | | Mean (SEM) | | Mean (SEM) | | Mean (SEM) | |
| DT-000414 | 22.4 | (1) | 11.7 | (0.7) | 8.8 | (0.8) | 5.7 | (0.7) | 3.4 | (0.2) |
| DT-000904 | 112.8 | (5.4) | 26.3 | (0.5) | 18.9 | (1.7) | 12.8 | (0.3) | 7.9 | (1) |
| DT-000905 | 24.4 | (0.8) | 17.5 | (4.4) | 15.3 | (1.8) | 12.5 | (1.7) | 13.3 | (1.8) |
| DT-000906 | 17.3 | (0.5) | 9.3 | (0.5) | 8.2 | (0.6) | 7.8 | (1.5) | 4.2 | (0.4) |
| DT-000907 | 19.6 | (1.7) | 12.2 | (1.9) | 11.1 | (0.9) | 7.2 | (0.2) | 5 | (0.4) |
| DT-000908 | 14.5 | (1) | 7.5 | (1) | 6.5 | (0.9) | 4.6 | (0.7) | 3.8 | (0.5) |
| DT-000909 | 50 | (3.5) | 14.2 | (4.3) | 12.3 | (5.4) | 3.7 | (0.2) | 2.7 | (0.6) |
| DT-000911 | 17.1 | (1.3) | 13 | (0.6) | 9 | (1) | 6 | (0.2) | 4.3 | (0.5) |
| DT-000912 | 16.6 | (1) | 11.8 | (0.8) | 9.5 | (0.4) | 7.6 | (0.6) | 6.3 | (0.3) |
| DT-000913 | 38.9 | (3.1) | 23.1 | (1.8) | 15 | (0.3) | 12.8 | (0.6) | 9.3 | (0.6) |
| DT-000914 | 20.4 | (3) | 13.1 | (1.7) | 8.7 | (0.6) | 6.2 | (0.6) | 4.1 | (0.5) |
| DT-000915 | 37.4 | (3.1) | 27.8 | (2) | 23.6 | (2) | 16.5 | (0.6) | 11.5 | (0.8) |
| DT-000916 | 45.7 | (4.5) | 26.4 | (2) | 16.9 | (1.7) | 10.1 | (0.6) | 8.9 | (0.6) |
| DT-000917 | 48 | (8.6) | 32.9 | (2.4) | 22.6 | (3.2) | 16.1 | (0.8) | 8.7 | (1.2) |
| DT-000919 | 40.3 | (4.5) | 24.3 | (2.2) | 19.3 | (2.7) | 16.1 | (1.7) | 18.3 | (3.2) |
| DT-000920 | 59.8 | (5.3) | 29.5 | (4) | 20.6 | (0.5) | 16.8 | (2) | 13.2 | (1.1) |
| DT-000923 | 74.9 | (6.8) | 44.3 | (4) | 33.1 | (5) | 28 | (5.7) | 15.9 | (3.9) |
| DT-000926 | 74.9 | (7.9) | 50.4 | (6.7) | 40 | (4.4) | 33.4 | (4.8) | 30 | (2.6) |
| DT-000928 | 28.1 | (1.61) | 17.5 | (3.1) | 11.6 | (0.9) | 10.4 | (2) | 7.7 | (0.9) |

TABLE 18
Transfection of siRNAs into Schwann Cells: Dose Response

| | PMP22 mRNA % Remaining | | | | | |
|---|---|---|---|---|---|---|
| | 0.3 nM | | 3 nM | | 30 nM | |
| Treatment | Mean | S.E.M. | Mean | S.E.M. | Mean | S.E.M. |
| DT-001103 | 84.3 | 3.5 | 73.8 | 21.7 | 63.7 | 30.8 |
| DT-000876 | 58.7 | 4.3 | 20.0 | 4.5 | 16.7 | 4.1 |
| DT-001104 | 19.9 | 4.1 | 8.8 | 1.2 | 6.6 | 0.2 |
| DT-001105 | 125.8 | 11.8 | 89.8 | 4.4 | 76.2 | 4.2 |
| DT-001107 | 31.3 | 5.6 | 12.1 | 0.8 | 8.9 | 1.3 |
| DT-000928 | 16.6 | 1.4 | 7.0 | 0.8 | 9.6 | 1.4 |
| DT-001106 | 15.6 | 0.9 | 5.4 | 0.7 | 3.3 | 0.3 |
| DT-000913 | 40.9 | 2.1 | 13.0 | 0.4 | 12.7 | 1.2 |
| DT-000914 | 14.6 | 1.7 | 5.4 | 0.8 | 4.8 | 0.8 |
| DT-001108 | 13.9 | 1.1 | 5.1 | 0.2 | 3.3 | 0.3 |
| DT-000408 | 130.1 | 12.11 | 100.4 | 2.2 | 114.6 | 6.5 |
| DT-000923 | 48.8 | 3.7 | 10.4 | 1.3 | 4.2 | 0.1 |
| DT-000873 | — | — | 11.7 | 0.5 | 3.4 | 0.2 |

Based on transfection data, certain compounds were identified as “hits” and selected for conjugation. Table 19 illustrates the parent unconjugated siRNAs identified as “hits” and the one or more conjugated siRNAs derived therefrom. Also shown are the lengths of the sense strand, the uptake motif attached to the sense strand, and the 5′ terminal moiety of the antisense strand.

TABLE 19
Unconjugated and conjugated siRNA relationship charts

| | Conjugated siRNA Compounds | | | | | | | |
|---|---|---|---|---|---|---|---|---|
| Unconj. | 19-mer | 19-mer | 19-mer | 19-mer | 21-mer | 21-mer | 21-mer | 21-mer |
| siRNA | DTx- | DTx- | DTx- | DTX- | DTx- | DTX- | DTX- | DTx- |
| 19-mer | 01-08 | 01-08 | 01-32 | 01-32 | 01-08 | 01-08 | 01-32 | 01-32 |
| 5′-PO4 | 5′-PO4 | 5′-VP | 5′-PO4 | 5′-VP | 5′-PO4 | 5′-VP | 5′-PO4 | 5′-VP |
| DT- | DT- | — | — | — | — | — | — | — |
| 000405 | 000544 | | | | | | | |
| DT- | DT- | — | — | — | — | — | — | — |
| 000408 | 001162 | | | | | | | |
| DT- | DT- | — | DT- | — | — | — | — | — |
| 000410 | 000545 | | 000620 | | | | | |
| DT- | DT- | — | — | — | — | — | — | — |
| 000412 | 000546 | | | | | | | |
| DT- | DT- | — | — | — | — | — | — | — |
| 000396 | 000621 | | | | | | | |
| DT- | DT- | — | — | — | — | — | — | — |
| 000398 | 000622 | | | | | | | |
| DT- | DT- | DT- | — | — | DT- | DT- | — | DT- |
| 000414 | 000623 | 000811 | | | 000945 | 000812 | | 001037 |
| | | | | | | DT- | | |
| | | | | | | 001246 | | |
| | | | | | | DT- | | |
| | | | | | | 001247 | | |
| | | | | | | DT- | | |
| | | | | | | 001250 | | |
| | | | | | | DT- | | |
| | | | | | | 001251 | | |
| | | | | | | DT- | | |
| | | | | | | 001252 | | |
| | | | | | | DT- | | |
| | | | | | | 001253 | | |
| | | | | | | DT- | | |
| | | | | | | 001254 | | |
| | | | | | | DT- | | |
| | | | | | | 001255 | | |
| | | | | | | DT- | | |
| | | | | | | 001256 | | |
| | | | | | | DT- | | |
| | | | | | | 001257 | | |
| | | | | | | DT- | | |
| | | | | | | 001858 | | |
| | | | | | | DT- | | |
| | | | | | | 001859 | | |
| | | | | | | DT- | | |
| | | | | | | 001860 | | |
| DT- | DT- | — | — | — | — | — | — | — |
| 000418 | 000624 | | | | | | | |
| DT- | DT- | — | — | — | — | — | — | — |
| 000420 | 000625 | | | | | | | |
| DT- | DT- | — | — | — | — | — | — | — |
| 000421 | 000626 | | | | | | | |
| DT- | DT- | — | — | — | — | — | — | — |
| 000422 | 000627 | | | | | | | |
| DT- | DT- | — | — | — | — | — | — | — |
| 000425 | 000628 | | | | | | | |
| DT- | DT- | — | — | — | DT- | — | — | — |
| 000845 | 000959 | | | | 001276 | | | |
| DT- | DT- | — | — | — | DT- | — | — | — |
| 000846 | 000960 | | | | 001277 | | | |
| DT- | DT- | — | — | — | DT- | DT- | — | — |
| 000847 | 000961 | | | | 001278 | 001844 | | |
| | | | | | | DT- | | |
| | | | | | | 001845 | | |
| DT- | DT- | — | — | — | DT- | — | — | — |
| 000848 | 001176 | | | | 001279 | | | |
| DT- | DT- | — | — | — | DT- | DT- | — | — |
| 000849 | 000962 | | | | 001280 | 001846 | | |
| | | | | | | DT- | | |
| | | | | | | 001847 | | |
| DT- | DT. | DT- | — | — | DT- | DT- | — | — |
| 000850 | 001177 | 001190 | | | 001281 | 001261 | | |
| DT- | DT- | DT- | — | — | DT- | DT- | — | — |
| 000852 | 001178 | 001191 | | | 001282 | 001262 | | |
| DT- | DT- | — | — | — | DT- | DT- | — | — |
| 000853 | 000963 | | | | 001283 | 001263 | | |
| DT- | DT- | DT- | — | — | DT- | DT- | — | — |
| 000855 | 001179 | 001192 | | | 001296 | 001848 | | |
| | | | | | | DT- | | |
| | | | | | | 001849 | | |
| DT- | DT- | DT- | — | — | DT- | DT- | — | — |
| 000856 | 001180 | 001193 | | | 001297 | 001358 | | |
| DT- | DT- | DT- | — | — | DT- | DT- | — | — |
| 000858 | 001181 | 001194 | | | 001298 | 001359 | | |
| DT- | DT- | — | — | — | DT- | DT- | — | — |
| 000865 | 000964 | | | | 001299 | 001264 | | |
| DT- | DT- | — | — | — | DT- | — | — | — |
| 000866 | 001182 | | | | 001300 | | | |
| DT- | DT- | — | — | — | DT- | DT- | — | — |
| 000868 | 001183 | | | | 001301 | 001360 | | |
| DT- | DT- | — | — | — | DT- | DT- | — | — |
| 000869 | 001184 | | | | 001302 | 001361 | | |
| DT- | DT- | — | — | — | DT- | DT- | — | — |
| 000872 | 001185 | | | | 001303 | 001362 | | |
| DT- | DT- | — | — | — | DT- | DT- | — | — |
| 000873 | 000965 | | | | 001304 | 001265 | | |
| DT- | DT- | — | — | — | DT | DT- | — | — |
| 000875 | 000966 | | | | 001305 | 001266 | | |
| DT- | DT- | — | — | — | DT- | — | — | — |
| 000876 | 000967 | | | | 001306 | | | |
| DT- | DT- | — | — | — | DT- | DT- | — | — |
| 000877 | 001186 | | | | 001307 | 001363 | | |
| DT- | DT- | — | — | DT- | DT- | DT | — | — |
| 000879 | 001195 | | | 001044 | 001322 | 001364 | | |
| DT- | DT- | — | — | DT- | DT- | DT- | — | — |
| 000880 | 001196 | | | 001045 | 001323 | 001365 | | |
| DT- | DT- | — | — | DT- | DT- | DT- | — | — |
| 000881 | 001197 | | | 001046 | 001324 | 001366 | | |
| DT- | DT- | DT- | — | DT- | DT- | DT- | — | — |
| 000882 | 001198 | 001038 | | 001039 | 001325 | 001367 | | |
| DT- | DT- | — | — | DT- | DT- | — | — | — |
| 000883 | 001199 | | | 001047 | 001326 | | | |
| DT- | DT- | — | — | DT- | DT- | — | — | — |
| 000884 | 001200 | | | 001048 | 001327 | | | |
| DT- | DT- | — | — | DT- | DT- | DT- | — | — |
| 000887 | 001201 | | | 001051 | 001328 | 001368 | | |
| DT- | DT- | — | — | DT- | DT- | — | — | — |
| 000892 | 001202 | | | 001053 | 001329 | | | |
| DT- | DT- | — | — | DT- | DT- | DT- | — | — |
| 000895 | 001203 | | | 001055 | 001330 | 001369 | | |
| DT- | DT- | — | — | DT- | DT- | — | — | — |
| 000896 | 001204 | | | 001056 | 001331 | | | |
| DT- | DT- | — | — | DT- | DT- | — | — | — |
| 000897 | 001205 | | | 001057 | 001332 | | | |
| DT- | DT- | — | — | DT- | DT- | — | — | — |
| 000900 | 001206 | | | 001059 | 001333 | | | |
| DT- | DT- | — | — | DT- | DT- | DT- | — | — |
| 000901 | 001207 | | | 001060 | 001334 | 001842 | | |
| | | | | | | DT- | | |
| | | | | | | 001843 | | |
| DT- | DT- | — | — | DT- | DT- | — | — | — |
| 000902 | 001208 | | | 001061 | 001335 | | | |
| DT- | — | DT- | DT- | DT- | DT- | DT- | — | — |
| 000905 | | 001145 | 001109 | 001121 | 001217 | 001221 | | |
| DT- | — | DT- | DT- | DT- | DT- | DT- | — | — |
| 000906 | | 001146 | 001110 | 001122 | 001218 | 001222 | | |
| DT- | — | DT- | DT- | DT- | DT- | — | — | — |
| 000907 | | 001149 | 001111 | 001123 | 001344 | | | |
| DT- | — | DT- | DT- | DT- | DT- | DT- | — | — |
| 000908 | | 001147 | 001112 | 001124 | 001219 | 001223 | | |
| DT- | — | DT- | DT- | DT- | DT- | — | — | — |
| 000909 | | 001150 | 001113 | 001125 | 001345 | | | |
| DT- | — | DT- | DT- | DT- | DT- | — | — | — |
| 000911 | | 001151 | 001114 | 001126 | 001346 | | | |
| DT- | — | DT- | DT- | DT- | DT- | — | — | — |
| 000912 | | 001152 | 001115 | 001127 | 001347 | | | |
| DT- | — | DT- | DT- | DT- | DT- | — | — | — |
| 000913 | | 001153 | 001116 | 001128 | 001348 | | | |
| DT- | — | DT- | DT- | DT- | DT- | DT- | — | — |
| 000914 | | 001148 | 001117 | 001129 | 001220 | 001224 | | |
| DT- | — | DT- | DT- | DT- | DT- | — | — | — |
| 000915 | | 001154 | 001118 | 001130 | 001349 | | | |
| DT- | — | DT- | DT- | DT- | DT- | — | — | — |
| 000916 | | 001155 | 001119 | 001131 | 001350 | | | |
| DT- | DT- | — | — | — | DT- | DT- | — | — |
| 000917 | 001230 | | | | 001234 | 001239 | | |
| DT- | DT- | — | — | — | DT- | DT- | — | — |
| 000919 | 001231 | | | | 001235 | 001240 | | |
| DT- | DT- | — | — | — | DT- | DT- | — | — |
| 000923 | 001161 | | | | 001236 | 001241 | | |
| DT- | DT- | — | — | — | DT- | DT- | — | — |
| 000925 | 001232 | | | | 001237 | 001242 | | |
| DT- | DT- | — | — | — | DT- | DT- | — | — |
| 000927 | 001233 | | | | 001238 | 001243 | | |
| DT- | — | DT- | DT- | DT- | DT- | — | — | — |
| 000928 | | 001156 | 001120 | 001132 | 001351 | | | |
| DT- | DT- | — | — | — | DT- | — | — | — |
| 001030 | 001187 | | | | 001355 | | | |
| DT- | DT- | — | — | — | DT- | — | — | — |
| 001031 | 001188 | | | | 001356 | | | |
| DT- | DT- | — | — | — | DT- | — | — | — |
| 001032 | 001189 | | | | 001357 | | | |

### Example 6: Free Uptake Experiments

Conjugated compounds were tested for their ability to inhibit the expression of PMP22 in HER cells engineered to express human PMP22 (HEK-PMP22 cells). These studies were performed under free uptake conditions as described herein. The “parent” unconjugated compound ID is indicated next to each conjugated compound ID.

Schwann cells and HEK-PMP22 cells were treated with siRNAs as indicated in the Tables below. RNA was isolated 48 hours later, reverse transcribed to cDNA and PMP22 expression was quantified by qPCR. The average PMP22 expression for each of four replicates was calculated and shown in Tables 20 through 34.

TABLE 20
Free Uptake of PMP22 siRNAs into HEK-PMP22 Cells

| | | PMP22 mRNA % Remaining | | | |
|---|---|---|---|---|---|
| | | 300 nM | | 3000 nM | |
| Conjugate | Parent | Mean | S.E.M. | Mean | S.E.M. |
| | DT-000405 | 103.6 | 3.7 | 97.8 | 0.8 |
| DT-000544 | DT-000405 | 98.9 | 0.6 | 94.4 | 2.0 |
| | DT-000410 | 108.8 | 10.8 | 119.1 | 7.9 |
| DT-000545 | DT-000410 | 69.0 | 1.4 | 26.9 | 0.3 |
| | DT-000412 | 96.7 | 2.6 | 96.7 | 0.3 |
| DT-000546 | DT-000412 | 74.7 | 1.4 | 62.1 | 1.3 |

TABLE 21
Free Uptake of PMP22 siRNAs into HEK-PMP22 Cells

| | | PMP22 mRNA % Remaining | | | | | | | |
|---|---|---|---|---|---|---|---|---|---|
| | | 100 nM | | 300 nM | | 1000 nM | | 3000 nM | |
| Conjugate | Parent | Mean (SEM) | | Mean (SEM) | | Mean (SEM) | | Mean (SEM) | |
| DT-000545 | DT-000410 | — | (—) | 42.9 | (1.8) | 1.3 | (42.9) | 24.5 | (1.2) |
| | DT-000396 | — | (—) | 102.7 | (1.9) | 98.3 | (1.6) | 93.8 | (1.6) |
| DT-000621 | DT-000396 | 72.7 | (0.7) | 69.2 | (1.3) | 57.3 | (2.4) | 60.7 | (3.9) |
| DT-000622 | DT-000398 | 63.3 | (2.1) | 39.8 | (6.4) | 13.1 | (0.4) | 11.1 | (0.2) |
| DT-000623 | DT-000414 | 62.2 | (6) | 22.5 | (2) | 7 | (0.4) | 2 | (0.3) |
| DT-000624 | DT-000418 | 51.2 | (2.1) | 25 | (1.4) | 12.6 | (0.1) | 12.2 | (0.4) |
| DT-000625 | DT-000420 | 67.5 | (3.6) | 32.3 | (1.5) | 11.8 | (0.5) | 7.4 | (0.1) |
| DT-000626 | DT-000421 | 84.7 | (3) | 61.8 | (2.5) | 33 | (1.5) | 19.8 | (0.7) |
| DT-000627 | DT-000422 | 89.3 | (1.4) | 70.2 | (1.3) | 39.3 | (2.1) | 27.3 | (0.4) |

TABLE 22
Free Uptake of PMP22 siRNAs into Schwann Cells

| | | PMP22 mRNA % Remaining | | | | | | | |
|---|---|---|---|---|---|---|---|---|---|
| | | 100 nM | | 300 nM | | 1000 nM | | 3000 nM | |
| Conjugate | Parent | Mean (SEM) | | Mean (SEM) | | Mean (SEM) | | Mean (SEM) | |
| DT-000337 | | — | | — | | 94.4 | (6.5) | 97.2 | (6.5) |
| DT-000545 | DT-000410 | — | | 77.4 | (5.5) | 58.3 | (3.3) | 44.7 | (2.9) |
| | DT-000396 | — | | — | | — | | 92.8 | (5.3) |
| DT-000621 | DT-000396 | 92.1 | (9) | 84.2 | (10.3) | 82.6 | (11.5) | 78.5 | (9.9) |
| DT-000622 | DT-000398 | 71.5 | (1.7) | 69.5 | (2.3) | 60.6 | (2.7) | 24.4 | (2) |
| DT-000623 | DT-000414 | 66.1 | (3.8) | 41 | (4.1) | 17.5 | (2.2) | 4.6 | (0.4) |
| DT-000624 | DT-000418 | 85.3 | (2.9) | 59.6 | (3.8) | 32.8 | (3.4) | 18.6 | (2.5) |
| DT-000625 | DT-000420 | 91.1 | (5.5) | 56.2 | (2.1) | 35.6 | (2.8) | 14 | (1.8) |
| DT-000626 | DT-000421 | 89.5 | (4.8) | 84.7 | (6) | 74.3 | (4.3) | 38.8 | (3.2) |
| DT-000627 | DT-000422 | 98 | (3.6) | 83.8 | (4.1) | 75.2 | (4.2) | 56.6 | (4.1) |
| DT-000628 | DT-000425 | 92.4 | (6.8) | 86.9 | (3.7) | 66.9 | (2.4) | 39.8 | (3) |

TABLE 23
Free Uptake of PMP22 siRNAs into HEK-PMP22 Cells

| | | PMP22 mRNA % Remaining | | | | |
|---|---|---|---|---|---|---|
| | | 30 | 100 | 300 | 1000 | 3000 |
| | | nM | nM | nM | nM | nM |
| | | Mean | Mean | Mean | Mean | Mean |
| Conjugate | Parent | (SEM) | (SEM) | (SEM) | (SEM) | (SEM) |
| DT- | DT- | 92.7 | 68.6 | 30.3 | 12.3 | 7.3 |
| 000623 | 000414 | (3.6) | (3) | (1.1) | (0.4) | (0.3) |
| DT- | DT- | 100.1 | 107 | 102.1 | 52.5 | 31.5 |
| 000959 | 000845 | (1.5) | (3.4) | (10.9) | (1.8) | (2.4) |
| DT- | DT- | 102.2 | 92.3 | 74.2 | 42.1 | 28.1 |
| 000960 | 000846 | (3.8) | (6.3) | (0.7) | (1.8) | (3.2) |
| DT- | DT- | 101.1 | 106.6 | 99.4 | 59.1 | 32.2 |
| 000961 | 000847 | (4.7) | (5.8) | (2.9) | (6.9) | (1.8) |
| DT- | DT- | 107 | 97.7 | 83.1 | 42.7 | 19.8 |
| 000962 | 000849 | (3.6) | (3.1) | (4.3) | (2.6) | (0.7) |
| DT- | DT- | 99.9 | 88.5 | 56.8 | 23.5 | 16.4 |
| 000963 | 000853 | (1.9) | (5.5) | (4.4) | (0.6) | (0.9) |
| DT- | DT- | 103.4 | 90.3 | 87.1 | 40.5 | 14.5 |
| 000964 | 000865 | (3.1) | (1.6) | (4.6) | (3.3) | (0.4) |
| DT- | DT- | 108.4 | 97.9 | 85.9 | 41.4 | 29.2 |
| 000965 | 000873 | (6.1) | (5.1) | (6.5) | (3.7) | (0) |
| DT- | DT- | 119.2 | 104.6 | 77.6 | 28.5 | 15.4 |
| 000966 | 000875 | (4.9) | (2.2) | (5.5) | (0.5) | (0.7) |
| DT- | DT- | 98.5 | 95.5 | 84.2 | 45.8 | 22 |
| 000967 | 000876 | (3) | (3.2) | (2.2) | (2.4) | (0.9) |

TABLE 24
Free Uptake of PMP22 siRNAs into Schwann Cells

| | | PMP22 mRNA % Remaining | | | | |
|---|---|---|---|---|---|---|
| | | 30 | 100 | 300 | 1000 | 3000 |
| | | nM | nM | nM | nM | nM |
| | | Mean | Mean | Mean | Mean | Mean |
| Conjugate | Parent | (SEM) | (SEM) | (SEM) | (SEM) | (SEM) |
| DT- | DT- | 102.7 | 89.2 | 67.9 | 31.1 | 14.5 |
| 000623 | 000414 | (3.7) | (3.2) | (1.7) | (1.3) | (0.7) |
| DT- | DT- | 92.7 | 92.6 | 87.9 | 71.4 | 50.8 |
| 000959 | 000845 | (3) | (7.5) | (5.1) | (0.3) | (2.8) |
| DT- | DT- | 99.8 | 93 | 104.7 | 82.2 | 73.1 |
| 000960 | 000846 | (5.8) | (2.4) | (6.9) | (4.9) | (2.2) |
| DT- | DT- | 113.3 | 112.1 | 112.4 | 102.1 | 69.8 |
| 000961 | 000847 | (5.4) | (2.3) | (8.8) | (3.8) | (1.9) |
| DT- | DT- | 120.4 | 116.9 | 103.2 | 90.1 | 64.6 |
| 000962 | 000849 | (3.7) | (5.7) | (7.7) | (2.5) | (3.6) |
| DT- | DT- | 94.1 | 95.2 | 96.7 | 93.1 | 81.9 |
| 000963 | 000853 | (2.2) | (2.6) | (2.5) | (2.1) | (2.8) |
| DT- | DT- | 120.9 | 112.9 | 97.1 | 63.6 | 46.7 |
| 000964 | 000865 | (2.3) | (5.6) | (7) | (0.9) | (1.6) |
| DT- | DT- | 108.2 | 107.2 | 113.9 | 115.1 | 109.8 |
| 000965 | 000873 | (6.9) | (4.4) | (7.4) | (14.7) | (2.1) |
| DT- | DT- | 113.3 | 105.3 | 105.8 | 81 | 71.7 |
| 000966 | 000875 | (5.7) | (2.4) | (6.6) | (8.2) | (4.2) |
| DT- | DT- | 90.1 | 98.1 | 111.6 | 100.8 | 81.3 |
| 000967 | 000876 | (10) | (6.3) | (3.2) | (3.7) | (2.5) |

TABLE 25
Free Uptake of PMP22 siRNAs into Schwann Cells

| | | PMP22 mRNA % Remaining | | | | | | | |
|---|---|---|---|---|---|---|---|---|---|
| | | 30 nM | | 100 nM | | 1000 nM | | 3000 nM | |
| Compound | Parent | Mean (SEM) | | Mean (SEM) | | Mean (SEM) | | Mean (SEM) | |
| DT-000623 | DT-000414 | 79.1 | (4.9) | 34.1 | (3.6) | 3.3 | (34.1) | 8.9 | (0.9) |
| DT-001038 | DT-000882 | 82.6 | (1.7) | 80.6 | (1.9) | 79.1 | (4.4) | 65.5 | (4.4) |
| DT-001039 | DT-000882 | 91.9 | (2.7) | 79.6 | (0.7) | 77.4 | (3.1) | 65.3 | (2.8) |
| DT-001045 | DT-000880 | 81.7 | (1.9) | 80.3 | (3) | 80.8 | (5.5) | 82.3 | (8.9) |
| DT-001048 | DT-000884 | 91.4 | (3.5) | 99.9 | (6.3) | 80.9 | (1.6) | 68.1 | (3.5) |
| DT-001051 | DT-000887 | 101.6 | (10.1) | 113.5 | (1.7) | 108.8 | (3.5) | 97.4 | (3.2) |
| DT-001057 | DT-000897 | 114.5 | (6.5) | 105.3 | (6.4) | 97.2 | (4.3) | 87.3 | (4.9) |
| DT-001059 | DT-000900 | 80.4 | (5.1) | 84.4 | (5.5) | 83.2 | (3.8) | 73.9 | (1.8) |
| DT-001060 | DT-000901 | 95.3 | (3.2) | 91.2 | (3.7) | 72.1 | (1.6) | 50.9 | (1) |
| DT-001061 | DT-000902 | 107.8 | (6.2) | 93.5 | (3.7) | 111.1 | (9) | 78.9 | (3.2) |

TABLE 26
Free Uptake of PMP22 siRNAs into Schwann Cells

| | | PMP22 mRNA % Remaining | | | | |
|---|---|---|---|---|---|---|
| | | 30 | 100 | 300 | 1000 | 3000 |
| | | nM | nM | nM | nM | nM |
| | | Mean | Mean | Mean | Mean | Mean |
| Compound | Parent | (SEM) | (SEM) | (SEM) | (SEM) | (SEM) |
| DT- | DT- | 94.8 | 79.8 | 50.7 | 17.5 | 3.4 |
| 000812 | 000414 | (3.6) | (2.2) | (1.8) | (0.5) | (0.2) |
| DT- | DT- | 86.8 | 84.8 | 75.7 | 54.1 | 30.4 |
| 001121 | 000905 | (8.6) | (1) | (1.7) | (0.6) | (1.5) |
| DT- | DT- | 95 | 89.1 | 71.8 | 40.6 | 21.9 |
| 001122 | 000906 | (1) | (3.9) | (1.7) | (0.4) | (0.9) |
| DT- | DT- | 101 | 94.4 | 80.5 | 63.5 | 52.9 |
| 001123 | 000907 | (1.9) | (3.3) | (1.9) | (2.3) | (2.1) |
| DT- | DT- | 100.8 | 85.2 | 76.3 | 47 | 20.7 |
| 001124 | 000908 | (3.9) | (8.5) | (1.8) | (1.2) | (1) |
| DT- | DT- | 93.2 | 102.2 | 92.6 | 81.2 | 52.7 |
| 001125 | 000909 | (5.4) | (2.4) | (3.7) | (1.3) | (1.2) |
| DT- | DT- | 119.2 | 108.5 | 100.9 | 75.3 | 50.1 |
| 001126 | 000911 | (3.3) | (3.9) | (0.9) | (3.7) | (2.8) |
| DT- | DT- | 98.8 | 103.9 | 89.7 | 61.3 | 37.7 |
| 001127 | 000912 | (1.3) | (2.3) | (2.8) | (1.9) | (1.3) |
| DT- | DT- | 105.9 | 100.7 | 100.9 | 87.8 | 69.9 |
| 001128 | 000913 | (5.3) | (7.7) | (4.4) | (4.2) | (5.2) |
| DT- | DT- | 97.7 | 91.8 | 78.1 | 50.4 | 26.8 |
| 001129 | 000914 | (5.3) | (3.9) | (2.3) | (2.3) | (0.4) |
| DT- | DT- | 97.5 | 98.1 | 97.7 | 77.3 | 54.4 |
| 001130 | 000915 | (2.8) | (1.5) | (1.1) | (3.9) | (0.7) |
| DT- | DT- | 109.8 | 105.4 | 100.2 | 81.6 | 50.8 |
| 001131 | 000916 | (2.9) | (4.5) | (3.5) | (3.7) | (7.6) |
| DT- | DT- | 94.6 | 97.3 | 93.8 | 71.4 | 43.3 |
| 001132 | 000928 | (2.7) | (1.9) | (3.1) | (3.5) | (1.6) |

TABLE 27
Free Uptake of PMP22 siRNAs into Schwann Cells

| | | PMP22 mRNA % Remaining | | | | |
|---|---|---|---|---|---|---|
| | | 30 | 100 | 300 | 1000 | 3000 |
| | | nM | nM | nM | nM | nM |
| | | Mean | Mean | Mean | Mean | Mean |
| Compound | Parent | (SEM) | (SEM) | (SEM) | (SEM) | (SEM) |
| DT- | DT- | 87.8 | 67.8 | 37.5 | 11.8 | 2.8 |
| 000812 | 000414 | (2.3) | (2.5) | (1.5) | (0.7) | (0.2) |
| DT- | DT- | 86.7 | 78.1 | 70.4 | 46.6 | 29 |
| 001037 | 000414 | (2.4) | (2.7) | (2.7) | (2.4) | (1.1) |
| DT- | DT- | 107.9 | 99.5 | 91.8 | 77.9 | 57.2 |
| 001121 | 000905 | (0.3) | (5.1) | (3.2) | (1.6) | (4.1) |
| DT- | DT- | 97.4 | 100.6 | 90.8 | 63.2 | 29.9 |
| 001145 | 000905 | (4.7) | (3.9) | (4.4) | (4.8) | (1.9) |
| DT- | DT- | 101.4 | 88.2 | 76.1 | 48.4 | 23.9 |
| 001122 | 000906 | (1.5) | (3.9) | (1.8) | (1) | (1.1) |
| DT- | DT- | 98.2 | 92.1 | 80.4 | 46.3 | 12.3 |
| 001146 | 000906 | (3.3) | (3.4) | (2.9) | (2.4) | (0.8) |
| DT- | DT- | 91.3 | 84.1 | 79.2 | 65.3 | 40.6 |
| 001124 | 000908 | (3.2) | (2.5) | (2.4) | (3) | (2.5) |
| DT- | DT- | 89.3 | 82.3 | 76.5 | 66.1 | 33.4 |
| 001147 | 000908 | (3) | (1.6) | (1.3) | (8.2) | (1.4) |
| DT- | DT- | 103.1 | 90.8 | 81 | 52.1 | 26.5 |
| 001129 | 000914 | (4.3) | (1.8) | (4.8) | (1.2) | (1.4) |
| DT- | DT- | 96.9 | 94.7 | 88.9 | 97.7 | 98.1 |
| 001148 | 000914 | (2.7) | (2.7) | (3.9) | (2.8) | (2.5) |
| DT- | DT- | 94.7 | 88.9 | 91.2 | 70.6 | 61.4 |
| 001123 | 000907 | (10.2) | (15) | (9.8) | (6.4) | (6.6) |
| DT- | DT- | 96.8 | 84.3 | 82.7 | 89 | 72.9 |
| 001149 | 000907 | (1.3) | (1.6) | (5.4) | (7.2) | (6.8) |
| DT- | DT- | 118.7 | 104.1 | 114.8 | 88.8 | 50.8 |
| 001125 | 000909 | (2.5) | (5.2) | (7.5) | (7.1) | (2.2) |
| DT- | DT- | 104.5 | 102.5 | 98.3 | 80.7 | 30.8 |
| 001150 | 000909 | (6.6) | (3.1) | (7.8) | (13.4) | (2.5) |

TABLE 28
Free Uptake of PMP22 siRNAs into HEK-PMP22 Cells

| | | PMP22 mRNA % Remaining | | | | |
|---|---|---|---|---|---|---|
| | | 30 | 100 | 300 | 1000 | 3000 |
| | | nM | nM | nM | nM | nM |
| | | Mean | Mean | Mean | Mean | Mean |
| Compound | Parent | (SEM) | (SEM) | (SEM) | (SEM) | (SEM) |
| DT- | DT- | 66.7 | 41.4 | 18.4 | 6.2 | 4.8 |
| 00812 | 000414 | (1.8) | (1.5) | (0.7) | (0.4) | (0.3) |
| DT- | DT- | 111.4 | 103.5 | 90.2 | 63.8 | 25.6 |
| 001176 | 000848 | (3.6) | (3.2) | (2.9) | (2.3) | (2.1) |
| DT- | DT- | 100.9 | 89.2 | 65 | 46.1 | 35.1 |
| 001177 | 000850 | (5.2) | (9.2) | (5.7) | (3.9) | (2.2) |
| DT- | DT- | 102.1 | 78.4 | 75 | 46.9 | 40.6 |
| 001190 | 000850 | (2.3) | (9.3) | (3.5) | (3.1) | (4.6) |
| DT- | DT- | 87.3 | 72.5 | 44.5 | 21 | 18.7 |
| 001178 | 000852 | (2.5) | (3.2) | (2.1) | (1.1) | (0.5) |
| DT- | DT- | 98 | 78.2 | 53.1 | 27.1 | 22.9 |
| 001191 | 000852 | (3.9) | (5.4) | (3.9) | (1.4) | (0.9) |
| DT- | DT- | 94.7 | 78.4 | 53.9 | 20.9 | 13.1 |
| 001179 | 000855 | (1.3) | (3.5) | (0.6) | (0.9) | (1.1) |
| DT- | DT- | 76.7 | 82.7 | 51.9 | 27.6 | 17.7 |
| 001192 | 000855 | (1.8) | (4.2) | (2.7) | (2.8) | (1.4) |
| DT- | DT- | 107.2 | 102.7 | 87.9 | 44.6 | 39.6 |
| 001180 | 000856 | (9.2) | (6.4) | (7.4) | (2.8) | (2.3) |
| DT- | DT- | 110.5 | 104.1 | 86.6 | 39.3 | 19.5 |
| 001193 | 000856 | (8.1) | (3.3) | (9.7) | (3) | (0.9) |
| DT- | DT- | 79.3 | 60.9 | 37.2 | 17.2 | 8.2 |
| 001181 | 000858 | (3.9) | (0.4) | (2.1) | (0.8) | (0.6) |
| DT- | DT- | 92.6 | 78.7 | 46.6 | 22.4 | 32.6 |
| 001194 | 000858 | (7.1) | (7.3) | (2.5) | (2) | (6.4) |

TABLE 29
Free Uptake of PMP22 siRNAs into Schwann Cells

| | | PMP22 mRNA % Remaining | | | | |
|---|---|---|---|---|---|---|
| | | 30 | 100 | 300 | 1000 | 3000 |
| | | nM | nM | nM | nM | nM |
| | | Mean | Mean | Mean | Mean | Mean |
| Compound | Parent | (SEM) | (SEM) | (SEM) | (SEM) | (SEM) |
| DT- | DT- | 99.9 | 83.7 | 59.3 | 26.7 | 7.4 |
| 00812 | 000414 | (5.8) | (8.4) | (6.2) | (2.2) | (0.6) |
| DT- | DT- | 82.8 | 82.2 | 89.5 | 84 | 82 |
| 001182 | 000866 | (8.5) | (7.1) | (5) | (6.4) | (5.3) |
| DT- | DT- | 98 | 90.7 | 86.6 | 59.5 | 25.8 |
| 001183 | 000868 | (10.5) | (5.3) | (8) | (2.8) | (0.9) |
| DT- | DT- | 89.9 | 85 | 100.3 | 95.7 | 92.4 |
| 001184 | 000869 | (4.7) | (4.3) | (12.8) | (8.4) | (9.4) |
| DT- | DT- | 85.3 | 84.8 | 95.2 | 111.8 | 60.7 |
| 001185 | 000872 | (5.9) | (8.1) | (8.4) | (3.7) | (4.3) |
| DT- | DT- | 145.2 | 142 | 131.7 | 82 | 26.4 |
| 001186 | 000877 | (12.1) | (12.1) | (3.8) | (4.5) | (1.2) |

TABLE 30
Free Uptake of PMP22 siRNAs into Schwann Cells

| | | PMP22 mRNA % Remaining | | | | |
|---|---|---|---|---|---|---|
| | | 30 | 100 | 300 | 1000 | 3000 |
| | | nM | nM | nM | nM | nM |
| | | Mean | Mean | Mean | Mean | Mean |
| Compound | Parent | (SEM) | (SEM) | (SEM) | (SEM) | (SEM) |
| DT- | DT- | 108 | 91.2 | 56.9 | 31.9 | 7.2 |
| 000812 | 000414 | (14.6) | (4.8) | (3.6) | (2.3) | (0.5) |
| DT- | DT- | 89.7 | 88.8 | 96.2 | 119 | 164.1 |
| 001187 | 0001030 | (15.5) | (16.9) | (16.1) | (14.2) | (17.8) |
| DT- | DT- | 152 | 143 | 145.6 | 139.4 | 104.9 |
| 001188 | 0001031 | (6.8) | (5.9) | (13.2) | (1.7) | (13.7) |
| DT- | DT- | 114.7 | 105.2 | 135.3 | 121.2 | 90.9 |
| 001189 | 0001032 | (8.4) | (9.8) | (7.8) | (17) | (13.6) |

TABLE 31
Free Uptake of PMP22 siRNAs into HEK-PMP22 Cells

| | | PMP22 mRNA % Remaining | | | | |
|---|---|---|---|---|---|---|
| | | 30 | 100 | 300 | 1000 | 3000 |
| | | nM | nM | nM | nM | nM |
| | | Mean | Mean | Mean | Mean | Mean |
| Compound | Parent | (SEM) | (SEM) | (SEM) | (SEM) | (SEM) |
| DT- | DT- | 84.7 | 46.9 | 20.3 | 10.2 | 5.5 |
| 000623 | 000414 | (3.1) | (2.3) | (0.8) | (0.4) | (0.2) |
| DT- | DT- | 82.9 | 50.1 | 20.8 | 9.8 | 6.6 |
| 000811 | 000414 | (9.4) | (1.2) | (1.2) | (0.4) | (0.3) |
| DT- | DT- | 50.6 | 25.3 | 8.9 | 2.1 | 1.4 |
| 000812 | 000414 | (1.9) | (1.5) | (0.9) | (0.2) | (0) |
| DT- | DT- | 67.1 | 41.5 | 18.8 | 7.1 | 1.5 |
| 000945 | 000414 | (1) | (2) | (1.2) | (0.6) | (0.1) |

TABLE 32
Free Uptake of PMP22 siRNAs into HEK-PMP22 Cells

| | | PMP22 mRNA % Remaining | | | | |
|---|---|---|---|---|---|---|
| | | 30 | 100 | 300 | 1000 | 3000 |
| | | nM | nM | nM | nM | nM |
| | | Mean | Mean | Mean | Mean | Mean |
| Compound | Parent | (SEM) | (SEM) | (SEM) | (SEM) | (SEM) |
| DT- | DT- | 77.8 | 80.6 | 67.7 | 35.6 | 10.2 |
| 000623 | 000414 | (1.5) | (6) | (5.8) | (3.7) | (1.7) |
| DT- | DT- | 80.7 | 76.9 | 63.9 | 42.3 | 15.2 |
| 000811 | 000414 | (2.4) | (1.6) | (1.4) | (3.1) | (1.8) |
| DT- | DT- | 82.2 | 74.9 | 58.8 | 16.3 | 3.1 |
| 000812 | 000414 | (3.6) | (6.9) | (9.2) | (1.4) | (0.9) |
| DT- | DT- | 89.1 | 95.6 | 64.6 | 31.1 | 6.6 |
| 000945 | 000414 | (2.9) | (11.2) | (3.6) | (3.6) | (0.7) |

TABLE 33
Free Uptake of PMP22 siRNAs into HEK-PMP22 Cells

| | | PMP22 mRNA % Remaining | | | | |
|---|---|---|---|---|---|---|
| | | 10 | 30 | 100 | 300 | 1000 |
| | | nM | nM | nM | nM | nM |
| | | Mean | Mean | Mean | Mean | Mean |
| Compound | Parent | (SEM) | (SEM) | (SEM) | (SEM) | (SEM) |
| DT- | DT- | 87.5 | 70.3 | 40.2 | 18.8 | 9.5 |
| 000812 | 000414 | (2.1) | (1.4) | (0.9) | (0.6) | (0.6) |
| DT- | DT- | 96.7 | 70.1 | 31.9 | 17.2 | 16.8 |
| 001246 | 000414 | (3.2) | (2.1) | (0.3) | (0.4) | (0.5) |
| DT- | DT- | 78.6 | 55.8 | 25.8 | 10.9 | 6.8 |
| 001247 | 000414 | (3.6) | (0.8) | (0.6) | (0.4) | (0.2) |
| DT- | DT- | 80.6 | 61.3 | 35.5 | 12.2 | 3.8 |
| 001250 | 000414 | (2.1) | (0.6) | (0.4) | (0.4) | (0.1) |
| DT- | DT- | 85.2 | 70.7 | 40.4 | 22 | 36.4 |
| 001251 | 000414 | (2.5) | (1.4) | (1.3) | (0.4) | (2) |
| DT- | DT- | 80.7 | 55.8 | 25 | 11.8 | 11.1 |
| 001252 | 000414 | (1.8) | (2.1) | (1.2) | (0.3) | (0.5) |
| DT- | DT- | 70.8 | 47.3 | 22.6 | 10.5 | 12.1 |
| 001253 | 000414 | (1.3) | (1.8) | (0.4) | (0.6) | (0.3) |
| DT- | DT- | 85.9 | 61.4 | 29.9 | 12.9 | 7.4 |
| 001254 | 000414 | (3.5) | (2.2) | (0.7) | (0.3) | (0.3) |
| DT- | DT- | 82.1 | 63.6 | 31.3 | 12.3 | 7 |
| 001255 | 000414 | (1.6) | (1.2) | (0.4) | (0.3) | (0.1) |
| DT- | DT- | 83.8 | 62.5 | 37.4 | 17.7 | 16.6 |
| 001256 | 000414 | (1.3) | (2.1) | (1.4) | (0.2) | (0.6) |
| DT- | DT- | 75.2 | 53.1 | 21.6 | 6.6 | 2.7 |
| 001257 | 000414 | (2.8) | (0.6) | (0.8) | (0.4) | (0.1) |
| DT- | DT- | 82 | 63.5 | 35.2 | 16.7 | 16.3 |
| 001258 | 000414 | (0.8) | (1.4) | (1.1) | (0.5) | (0.9) |
| DT- | DT- | 80.4 | 64.3 | 37 | 15.5 | 11.6 |
| 001259 | 000414 | (1.6) | (2.4) | (0.8) | (0.5) | (0.9) |
| DT- | DT- | 70.6 | 55.8 | 33 | 12.9 | 14.9 |
| 001260 | 000414 | (0.4) | (2.7) | (6.4) | (0.4) | (0.4) |

TABLE 34
Free Uptake of PMP22 siRNAs into Schwann Cells

| | | PMP22 mRNA % Remaining | | | | |
|---|---|---|---|---|---|---|
| | | 10 | 30 | 100 | 300 | 1000 |
| | | nM | nM | nM | nM | nM |
| | | Mean | Mean | Mean | Mean | Mean |
| | | (SEM) | (SEM) | (SEM) | (SEM) | (SEM) |
| DT- | DT- | 96.8 | 91.1 | 75 | 50.1 | 23.7 |
| 000812 | 000414 | (3) | (1.3) | (2.6) | (1.3) | (0.7) |
| DT- | DT- | 121.1 | 104.6 | 81.9 | 53.9 | 28.7 |
| 001246 | 000414 | (2.9) | (2.5) | (2.2) | (0.5) | (1) |
| DT- | DT- | 97.9 | 80.8 | 59.6 | 30.7 | 11.1 |
| 001247 | 000414 | (3.2) | (1.9) | (0.2) | (0.8) | (0.7) |
| DT- | DT- | 96.9 | 90.9 | 62.2 | 33.1 | 12.2 |
| 001250 | 000414 | (2.4) | (8.1) | (1.6) | (1.1) | (1.7) |
| DT- | DT- | 92.8 | 95.8 | 87.5 | 84.5 | 74.5 |
| 001251 | 000414 | (3.8) | (1.4) | (1.8) | (2.6) | (2.4) |
| DT- | DT- | 86.2 | 85.5 | 67.9 | 52.3 | 33 |
| 001252 | 000414 | (1.6) | (1.9) | (2.8) | (2.2) | (1.2) |
| DT- | DT- | 92 | 96.3 | 63.4 | 43.4 | 23 |
| 001253 | 000414 | (2.9) | (14.2) | (1.8) | (1) | (1.1) |
| DT- | DT- | 85.5 | 88.6 | 68.8 | 41.1 | 17.3 |
| 001254 | 000414 | (1.4) | (2.3) | (1.1) | (1.1) | (1.6) |
| DT- | DT- | 90 | 86.6 | 76.6 | 60 | 28.1 |
| 001255 | 000414 | (3) | (3.1) | (3.1) | (9.4) | (1.2) |
| DT- | DT- | 123.9 | 112 | 91 | 61.9 | 51 |
| 001256 | 000414 | (12) | (8.6) | (10.6) | (0.4) | (4.6) |
| DT- | DT- | 87 | 84.2 | 65.9 | 35.1 | 13.7 |
| 001257 | 000414 | (2) | (3.9) | (1.3) | (0.9) | (0.7) |
| DT- | DT- | 86.3 | 81.3 | 70.4 | 54.7 | 31.3 |
| 001258 | 000414 | (4.1) | (2.3) | (2.4) | (1.3) | (1.3) |
| DT- | DT- | 101 | 95.4 | 76.1 | 48.2 | 24.8 |
| 001259 | 000414 | (3.6) | (8.8) | (1.7) | (2.5) | (1) |
| DT- | DT- | 86.2 | 81.3 | 70.1 | 55.6 | 41.7 |
| 001260 | 000414 | (2.2) | (1.3) | (1.3) | (0.8) | (1.3) |

### Example 7: Target Engagement in Mice

Conjugated PMP22 siRNAs were tested in wild-type C₅₇BL/6J mice. In this experiment, control siRNAs were DT-000155 and DT-000337, both DTx-01-08-conjugated siRNAs targeting PTEN, each having a unique nucleotide sequence. Also tested was DT-000428, a fully phosphorothioated LNA gapmer antisense oligonucleotide (ASO) targeting PMP22, where a 10-nucleotide DNA gap is flanked by 3-nucleotide LNA wings (5′-A_{L}T_{L}C_{L}T_{D}T_{D}C_{D}A_{D}A_{D}T_{D}C_{D}A_{D}A_{D}C_{D}A_{L}G_{L}C_{L}-3′; subscript L is an LNA nucleotide and subscript D is a beta-D-deoxyribonucleotide; nucleotides four to 19 of SEQ ID NO: 591). Groups of five mice each were treated with PBS or compound at a dose of 30 mg/kg according to the dosing schedule indicated in Table 35. On Day 12, mice were sacrificed, and RNA was collected from tissue for RNA extraction and quantitation of mouse PMP22 mRNA levels by quantitative RT-PCR. The average percent expression in the central sciatic nerve was calculated for each treatment and is shown in Table 35.

TABLE 35
Mouse PMP22 mRNA expression in central
sciatic nerve of wild-type mice

| | Unconjugated | | PMP22 mRNA % | |
|---|---|---|---|---|
| | Parent (if | | remaining | |
| Treatment | applicable) | Doses | Mean | S.E.M. |
| PBS | | Days 1, 3, 5 | 101.7 | 9.8 |
| DT-000155 | | Days 1, 3, 5 | 93.5 | 10.0 |
| DT-000337 | | Days 1, 3, 5 | 87.1 | 7.0 |
| DT-000428 | | Days 1, 3, 5, 8, 10 | 66.6 | 8.4 |
| DT-000544 | DT-000405 | Days 1, 3, 5 | 87.1 | 7.9 |
| DT-000545 | DT-000410 | Days 1, 3, 5 | 73.8 | 3.8 |
| DT-000546 | DT-000412 | Days 1, 3, 5 | 84.3 | 3.8 |

C3-PMP22 mice express three to four copies of a wild-type human PMP22 gene and are used as an experimental model of CMT1A. Conjugated siRNAs targeted to human PMP22 were selected for their ability to reduce human PMP22 in C3-PMP22 mice. Experiments were performed as described herein.

In this experiment, the control siRNA was DT-000337, a DTx-01-08-conjugated siRNA targeting PTEN. Also tested was DT-000428, a fully phosphorothioated LNA gapmer antisense oligonucleotide targeting PMP22, where a 10-nucleotide DNA gap is flanked by 3-nucleotide LNA wings (5′-A_{L}T_{L}C_{L}T_{D}T_{D}C_{D}A_{D}A_{D}T_{D}C_{D}A_{D}A_{D}C_{D}A_{L}G_{L}C_{L}-3′; nucleotides 4 to 19 of SEQ ID NO: 438; subscript L is an LNA nucleotide and subscript D is a beta-D-deoxyribonucleotide). Groups of six mice each were treated with PBS, siRNA compound at a dose of 50 mg/kg, or DT-000428 at a dose of 100 mg/kg on Days 1, 7, and 14. On Day 21, mice were sacrificed, and RNA was collected from tissue for RNA extraction and quantitation of human PMP22 mRNA levels by quantitative RT-PCR. The average percent expression in the sciatic nerve and tibial nerve was calculated for each treatment and is shown in Table 36.

TABLE 36
Human PMP22 mRNA expression in central
sciatic nerve of C3-PMP22 mice

| | Unconjugated | PMP22 mRNA % remaining | | | |
|---|---|---|---|---|---|
| | Parent (if | Sciatic | | Tibial | |
| Treatment | applicable) | Mean | S.E.M. | Mean | S.E.M. |
| PBS | | 103.3 | 10.4 | 104 | 14.0 |
| DT-000337 | | 93.5 | 11.3 | 93.9 | 12.6 |
| DT-000622 | DT-000398 | 103.5 | 16.0 | 113.9 | 15.8 |
| DT-000623 | DT-000414 | 56.3 | 4.2 | 47.8 | 4.8 |
| DT-000625 | DT-000420 | 96.5 | 11.2 | 87.2 | 14.5 |
| DT-000428 | | 77.5 | 11.0 | 65.4 | 10.7 |

The most active compound from the above study, DT-000623, was further tested. Groups of six C3-PMP22 mice each were treated with PBS or DT-000623 siRNA compound for a total of 1 dose, 2 doses, or 3 doses, at the dosing schedule indicated in Table 37. For comparison, wild-type mice were treated with PBS on the same dosing schedule. After 21 days, mice were sacrificed, and RNA was collected from tissue for RNA extraction and quantitation of human PMP22 mRNA levels by quantitative RT-PCR. mRNA levels for the mouse sciatic nerve markers MPZ, Pou3F1, Sc5d, and Id2 were also calculated. The average percent expression for each mRNA in the sciatic nerve and tibial nerve was calculated for each treatment and is shown in Table 37. In each table, wild-type PBS indicates data collected from wild-type mice treated with PBS. All other data were obtained in C3-PMP22 mice.

TABLE 37
Human PMP22 and sciatic nerve marker mRNA expression
in sciatic and tibial nerves of C3-PMP22 mice
following 1, 2, or 3 doses of conjugated siRNA
Human PMP22 mRNA

| | | Sciatic Nerve | | Tibial Nerve | |
|---|---|---|---|---|---|
| Treatment | Dosing | Mean | S.E.M. | Mean | S.E.M. |
| PBS | | 104.3 | 3.0 | 114.1 | 6.2 |
| DT-000623 | Days 1, 7, 14 | 55.2 | 3.8 | 64.9 | 3.6 |
| DT-000623 | Days 7, 14 | 47.1 | 4.7 | 69.4 | 3.5 |
| DT-000623 | Day 14 | 58.8 | 5.9 | 73.4 | 9.2 |

Mouse MPZ mRNA

| | Dosing | Sciatic Nerve | | Tibial Nerve | |
|---|---|---|---|---|---|
| wild-type PBS | | 102.3 | 8.8 | 104.4 | 14.3 |
| PBS | | 65.8 | 2.0 | 63.3 | 4.2 |
| DT-000623 | Days 1, 7, 14 | 119.2 | 9.6 | 91.2 | 11.4 |
| DT-000623 | Days 7, 14 | 98.5 | 6.0 | 86.2 | 3.6 |
| DT-000623 | Day 14 | 91.6 | 6.1 | 76.9 | 5.4 |

| | Sciatic Nerve | | Tibial Nerve | |
|---|---|---|---|---|
| Dosing | Mean | S.E.M. | Mean | S.E.M. |

Mouse Pou3F1 mRNA

| | | | | | |
|---|---|---|---|---|---|
| wild-type PBS | | 101.8 | 8.5 | 146.1 | 67.1 |
| PBS | | 494.5 | 29.2 | 241.2 | 45.6 |
| DT-000623 | Days 1, 7, 14 | 417.7 | 37.1 | 258.3 | 23.6 |
| DT-000623 | Days 7, 14 | 290.2 | 30.8 | 221.7 | 15.9 |
| DT-000623 | Day 14 | 445.4 | 36.36 | 293.8 | 25.87 |

Mouse Sc5d mRNA

| | | | | | |
|---|---|---|---|---|---|
| wild-type PBS | | 100.6 | 4.6 | 105.5 | 13.0 |
| PBS | | 52.1 | 1.5 | 84.3 | 4.4 |
| DT-000623 | Days 1, 7, 14 | 84.1 | 5.8 | 118.6 | 13.3 |
| DT-000623 | Days 7, 14 | 85.5 | 10.1 | 99.7 | 6.4 |
| DT-000623 | Day 14 | 79.8 | 6.0 | 79.4 | 9.6 |

Mouse Id2 mRNA

| | | | | | |
|---|---|---|---|---|---|
| wild-type PBS | | 113.0 | 28.2 | 122.0 | 34.6 |
| PBS | | 465.1 | 30.0 | 143.6 | 16.5 |
| DT-000623 | Days 1, 7, 14 | 364.0 | 50.1 | 144.5 | 24.4 |
| DT-000623 | Days 7, 14 | 273.4 | 33.2 | 132.8 | 21.9 |
| DT-000623 | Day 14 | 402.8 | 49.3 | 329.8 | 55.9 |

DT-000623 and variants, DT-000811 and DT-000812, were tested in C3-PMP22 mice. Groups of five C3-PMP22 mice each were treated with PBS or a single dose of 10 mg/kg, 30 mg/kg, or 100 mg/kg of DT-000623, DT-000811 and DT-000812. On Day 7 following the single-dose administration, mice were sacrificed, and RNA was collected from tissue for RNA extraction and quantitation of human PMP22 mRNA levels by quantitative RT-PCR. The average percent expression for each gene in the sciatic nerve and tibial nerve was calculated for each treatment and is shown in Table 38.

TABLE 38
Human PMP22 mRNA expression in sciatic and tibial nerves
of C3-PMP22 mice seven days following 10 mg/kg, 30
mg/kg, or 100 mg/kg doses of conjugated siRNA

| | Vehicle | | 10 mg/kg | | 30 mg/kg | | 100 mg/kg | |
|---|---|---|---|---|---|---|---|---|
| Treatment | Mean | S.E.M. | Mean | S.E.M. | Mean | S.E.M. | Mean | S.E.M. |

Sciatic Nerve

| | | | | | | | | |
|---|---|---|---|---|---|---|---|---|
| PBS | 100.7 | 5.5 | — | — | — | — | — | — |
| DT-000623 | — | — | 84.9 | 2.9 | 71.2 | 7.4 | 55.2 | 5.2 |
| DT-000811 | — | — | 74.3 | 8.6 | 68.8 | 3.5 | 38.9 | 4.2 |
| DT-000812 | — | — | 58.4 | 1.7 | 54.6 | 4.7 | 19.9 | 1.6 |

Tibial Nerve

| | | | | | | | | |
|---|---|---|---|---|---|---|---|---|
| PBS | 100.5 | 4.9 | — | — | — | — | — | — |
| DT-000623 | — | — | 104.6 | 5.1 | 89.5 | 11.9 | 58.9 | 5.7 |
| DT-000811 | — | — | 93.9 | 5.9 | 79.3 | 4.9 | 36.9 | 5.3 |
| DT-000812 | — | — | 98.1 | 6.3 | 65.5 | 4.1 | 27.0 | 2.2 |

DT-000812 and DT-000945, an additional variant of DT-000623, were tested in C3-PMP22 mice. Groups of six C3-PMP22 mice each were treated with PBS or a single dose of 30 mg/kg of DT-000812 and DT-000945. One group of each treatment was sacrificed 14 days following the single-dose section, and second groups of each treatment were sacrificed 28 days following the single-dose injection. RNA was collected from tissue for RNA extraction. Human PMP22 mRNA expression was measured by quantitative RT-PCR for both endpoints. Mouse MPZ, Pou3Fl, and Sc5d mRNA levels were measured by quantitative RT-PCR for the 28-day endpoint. The average percent expression for each gene in the sciatic nerve, brachial plexus nerve, and tibial nerve was calculated for each treatment and time period and is shown in Tables 39 and 40.

TABLE 39
Human PMP22 mRNA expression in C3-PMP22 mice 14 and 28 days
following a single dose of 30 mg/kg conjugated siRNA
14 days Post-Injection

| | Sciatic | | Brachial Plexus | | Tibial | |
|---|---|---|---|---|---|---|
| Treatment | Mean | S.E.M. | Mean | S.E.M. | Mean | S.E.M. |
| PBS | 100.5 | 4.8 | 102.1 | 9.0 | 101.3 | 7.2 |
| DT-000812 | 46.3 | 5.4 | 31.5 | 5.9 | 61.0 | 5.4 |
| DT-000945 | 76.0 | 7.9 | 47.1 | 8.0 | 94.8 | 14.7 |

28 days Post-Injection

| | Sciatic | | Brachial Plexus | | Tibial | |
|---|---|---|---|---|---|---|
| | Mean | S.E.M. | Mean | S.E.M. | Mean | S.E.M. |
| PBS | 100.1 | 2.4 | 103.1 | 11.2 | 102.4 | 9.4 |
| DT-000812 | 36.9 | 8.1 | 39.5 | 8.5 | 65.2 | 6.2 |
| DT-000945 | 89.5 | 5.6 | 82.5 | 9.6 | 106.5 | 3.6 |

TABLE 40
Myelin-specific mRNA expression in C3-PMP22 mice 28 days
following a single dose of 30 mg/kg conjugated siRNA
MPZ expression

| | Sciatic | | Brachial Plexus | | Tibial | |
|---|---|---|---|---|---|---|
| Treatment | Mean | S.E.M. | Mean | S.E.M. | Mean | S.E.M. |
| PBS | 100.1 | 2.0 | 105.4 | 13.7 | 101.7 | 8.1 |
| DT-000812 | 136.3 | 9.4 | 212.5 | 25.8 | 119.6 | 10.2 |
| DT-000945 | 121.6 | 7.6 | 127.6 | 8.3 | 121.1 | 3.2 |

Pou3F1

| | Sciatic | | Brachial Plexus | | Tibial | |
|---|---|---|---|---|---|---|
| | Mean | S.E.M. | Mean | S.E.M. | Mean | S.E.M. |
| PBS | 103.9 | 12.4 | 109.3 | 19.5 | 103.6 | 12.1 |
| DT-000812 | 42.0 | 7.9 | 56.1 | 9.9 | 67.0 | 4.8 |
| DT-000945 | 73.3 | 8.5 | 61.6 | 9.4 | 116.1 | 13.4 |

Sc5d

| | Sciatic | | Brachial Plexus | | Tibial | |
|---|---|---|---|---|---|---|
| | Mean | S.E.M. | Mean | S.E.M. | Mean | S.E.M. |
| PBS | 100.9 | 6.0 | 101.7 | 7.9 | 101.3 | 7.0 |
| DT-000812 | 147.2 | 20.6 | 189.5 | 26.4 | 157.4 | 10.6 |
| DT-000945 | 134.3 | 10.1 | 146.7 | 15.2 | 159.9 | 9.6 |

### Example 8: In Vivo Screening of PMP22 siRNAs

To determine whether variations in siRNA nucleotide sequence and/or modified nucleotide pattern would yield compounds with improved properties such as potency and duration of action, further compounds targeting PMP22 were designed and tested. The structure of each compound is shown in Table 4.

Groups of four or five C3-PMP22 mice each were treated with PBS or a single dose of PBS or 30 mg/kg of conjugated siRNA compound. Seven days following injection, mice were sacrificed, and sciatic and brachial plexus nerves was collected for RNA extraction. Human PMP22 mRNA expression was measured by quantitative RT-PCR. The average percent expression for human PMP22 mRNA was calculated for each treatment and is shown in Table 41.

TABLE 41
Human PMP22 mRNA 7 days following a single injection
of 30 mg/kg of conjugated siRNA compound

| | |
|---|---|
| Sciatic | Brachial Plexus |

| Treatment | Mean | S.E.M. | Mean | S.E.M. |
|---|---|---|---|---|
| PBS | 100.1 | 2.1 | 101.4 | 8.0 |
| DT-000812 | 22.6 | 1.7 | 27.0 | 2.5 |
| DT-001037 | 50.8 | 2.1 | 77.6 | 7.0 |
| DT-001038 | 79.7 | 4.5 | 85.5 | 8.2 |
| DT-001039 | 83.8 | 5.4 | 91.3 | 10.1 |
| DT-001059 | 83.8 | 2.5 | 103.4 | 7.0 |
| DT-001060 | 74.7 | 4.9 | 112.6 | 17.6 |

Groups of six C3-PMP22 mice each were treated with PBS or a single dose of PBS or 50 mg/kg of conjugated siRNA compound. Seven days following injection, mice were sacrificed, and sciatic and brachial plexus nerves was collected for RNA extraction. Human PMP22 mRNA expression was measured by quantitative RT-PCR. The average percent expression for human PMP22 mRNA was calculated for each treatment and is shown in Tables 42 through 49. For the compounds in Table 49, only the % human PMP22 remaining in the sciatic nerve is shown. Each table represents a different experiment.

TABLE 42
Human PMP22 mRNA 7 days following a single injection
of 50 mg/kg of conjugated siRNA compound

| | |
|---|---|
| Sciatic | Brachial Plexus |

| Treatment | Mean | S.E.M. | Mean | S.E.M. |
|---|---|---|---|---|
| PBS | 102.4 | 10.6 | 101.5 | 8.0 |
| DT-000623 | 53.0 | 2.9 | 55.8 | 7.7 |
| DT-000964 | 69.0 | 2.5 | 86.5 | 5.7 |
| DT-000965 | 63.8 | 2.3 | 85.4 | 9.8 |
| DT-000966 | 62.8 | 2.0 | 86.7 | 5.4 |
| DT-000967 | 62.2 | 2.7 | 95.7 | 6.3 |

TABLE 43
Human PMP22 mRNA 7 days following a single injection
of 50 mg/kg of conjugated siRNA compound

| | |
|---|---|
| Sciatic | Brachial Plexus |

| Treatment | Mean | S.E.M. | Mean | S.E.M. |
|---|---|---|---|---|
| PBS | 100.6 | 5.2 | 100.4 | 3.9 |
| DT-000623 | 83.1 | 5.4 | 65.1 | 5.3 |
| DT-000959 | 114.6 | 7.0 | 109.3 | 10.5 |
| DT-000960 | 106.0 | 5.7 | 85.5 | 4.7 |
| DT-000961 | 113.2 | 6.2 | 86.1 | 4.4 |
| DT-000962 | 110.6 | 4.8 | 83.0 | 5.8 |
| DT-000963 | 100.3 | 2.7 | 62.1 | 5.5 |

TABLE 44
Human PMP22 mRNA 7 days following a single injection
of 50 mg/kg of conjugated siRNA compound

| | |
|---|---|
| Sciatic | Brachial Plexus |

| Treatment | Mean | S.E.M. | Mean | S.E.M. |
|---|---|---|---|---|
| PBS | 101 | 6.6 | 107.7 | 20.3 |
| DT-000812 | 32.3 | 3.6 | 23.3 | 3.6 |
| DT-001121 | 94.5 | 4.8 | 67.9 | 13.2 |
| DT-001122 | 83.1 | 1.3 | 69.1 | 14.0 |
| DT-001124 | 99.1 | 5.6 | 65.9 | 8.6 |
| DT-001129 | 84.5 | 3.4 | 91.6 | 10.3 |
| DT-001145 | 90.1 | 4.2 | 92.0 | 8.9 |
| DT-001146 | 76.4 | 3.1 | 64.9 | 7.3 |
| DT-001147 | 92.8 | 2.2 | 68.8 | 7.4 |
| DT-001148 | 91.8 | 5.2 | 77.4 | 6.9 |

TABLE 45
Human PMP22 mRNA 7 days following a single injection
of 50 mg/kg of conjugated siRNA compound

| | |
|---|---|
| Sciatic | Brachial Plexus |

| Treatment | Mean | S.E.M. | Mean | S.E.M. |
|---|---|---|---|---|
| PBS | 100.2 | 3.5 | 100.7 | 6.8 |
| DT-000812 | 32.5 | 7.6 | 20.6 | 6.3 |
| DT-001190 | 84.5 | 2.6 | 86.7 | 13.8 |
| DT-001191 | 85.5 | 5.5 | 110.5 | 15.4 |
| DT-001192 | 93.2 | 5.7 | 90.1 | 12.2 |
| DT-001193 | 87.6 | 1.7 | 94.5 | 6.2 |
| DT-001194 | 87.5 | 3.2 | 109.2 | 13.2 |

TABLE 46
Human PMP22 mRNA 7 days following a single injection
of 50 mg/kg of conjugated siRNA compound

| | |
|---|---|
| Sciatic | Brachial Plexus |

| Treatment | Mean | S.E.M. | Mean | S.E.M. |
|---|---|---|---|---|
| PBS | 100.6 | 5.6 | 102.4 | 9.7 |
| DT-000812 | 29.3 | 1.9 | 22.6 | 2.6 |
| DT-001221 | 92.2 | 3.7 | 82.3 | 4.9 |
| DT-001224 | 88.2 | 6.5 | 82.7 | 9.3 |
| DT-001223 | 85.7 | 3.8 | 86.4 | 6.6 |

TABLE 47
Human PMP22 mRNA 7 days following a single injection
of 50 mg/kg of conjugated siRNA compound

| | |
|---|---|
| Sciatic | Brachial Plexus |

| Treatment | Mean | S.E.M. | Mean | S.E.M. |
|---|---|---|---|---|
| PBS | 100.5 | 5.2 | 103.4 | 13.4 |
| DT-000812 | 37.1 | 7.4 | 26.5 | 4.0 |
| DT-001239 | 100.0 | 6.2 | 89.0 | 8.0 |
| DT-001240 | 104.2 | 6.1 | 101.7 | 6.6 |
| DT-001241 | 119.0 | 13.6 | 90.3 | 8.6 |
| DT-001242 | 102.2 | 8.7 | 91.9 | 7.3 |
| DT-001243 | 129.8 | 15.1 | 110.1 | 11.7 |
| DT-001261 | 73.7 | 4.4 | 53.7 | 6.8 |
| DT-001262 | 64.3 | 4.1 | 69.3 | 20.3 |
| DT-001263 | 43.2 | 2.9 | 28.6 | 3.5 |

TABLE 48
Human PMP22 mRNA 7 days following a single injection
of 50 mg/kg of conjugated siRNA compound

| | |
|---|---|
| Sciatic | Brachial Plexus |

| Treatment | Mean | S.E.M. | Mean | S.E.M. |
|---|---|---|---|---|
| PBS | 101.3 | 8.5 | 102.5 | 9.9 |
| DT-000812 | 31.4 | 3.1 | 41.8 | 6.2 |
| DT-001264 | 64.1 | 7.6 | 78.2 | 10.9 |
| DT-001265 | 82.0 | 5.7 | 98.4 | 9.1 |
| DT-001266 | 74.9 | 6.2 | 87.9 | 10.4 |

TABLE 49
Human PMP22 mRNA 7 days following a single injection
of 50 mg/kg of conjugated siRNA compound

Sciatic

| Treatment | Mean | S.E.M. |
|---|---|---|
| PBS | 100.9 | 6.0 |
| DT-000812 | 44.5 | 5.6 |
| DT-001358 | 91.2 | 3.8 |
| DT-001359 | 93.9 | 4.4 |
| DT-001360 | 91.7 | 2.1 |
| DT-001361 | 92.0 | 3.1 |
| DT-001362 | 102.3 | 5.4 |
| DT-001363 | 100.2 | 4.2 |
| DT-001364 | 106.5 | 6.7 |
| DT-001365 | 92.5 | 4.6 |
| DT-001366 | 89.4 | 4.6 |
| DT-001367 | 87.8 | 6.2 |
| DT-001368 | 87.2 | 3.0 |
| DT-001369 | 97.6 | 5.1 |

Groups of six C3-PMP22 mice each were treated with a single dose of PBS, or 10 mg/kg or 30 mg/kg of conjugated siRNA compound (except for DT-000812 which was dosed only at 30 mg/kg). At Day 14 following injection, mice were sacrificed, and sciatic and brachial plexus nerve tissues were harvested for RNA extraction. Human PMP22 mRNA expression was measured by quantitative RT-PCR. The average percent expression for human PMP22 mRNA was calculated for each treatment and is shown in Tables 50 through 52. Each table represents a separate experiment.

TABLE 50
Human PMP22 mRNA 14 days following a single injection
of 10 mg/kg or 30 mg/kg of conjugated siRNA compound

| | PBS | | 10 mg/kg | | 30 mg/kg | |
|---|---|---|---|---|---|---|
| Treatment | Mean | S.E.M. | Mean | S.E.M. | Mean | S.E.M. |

Sciatic Nerve

| | | | | | | |
|---|---|---|---|---|---|---|
| PBS | 100.9 | 6.0 | — | — | — | — |
| DT-000812 | — | — | — | — | 47.1 | 4.7 |
| DT-001246 | — | — | 64.5 | 8.6 | 33.9 | 2.9 |
| DT-001247 | — | — | 57.8 | 4.2 | 37.2 | 1.9 |

Brachial Plexus

| | | | | | | |
|---|---|---|---|---|---|---|
| PBS | 101.3 | 6.9 | — | — | — | — |
| DT-000812 | — | — | — | — | 58.1 | 3.5 |
| DT-001246 | — | — | 74.1 | 7.9 | 44.2 | 2.6 |
| DT-001247 | — | — | 90.6 | 8.3 | 37.7 | 6.5 |

TABLE 51
Human PMP22 mRNA 14 days following a single injection
of 10 mg/kg or 30 mg/kg of conjugated siRNA compound
Sciatic Nerve

| | PBS | | 10 mg/kg | | 30 mg/kg | |
|---|---|---|---|---|---|---|
| Treatment | Mean | S.E.M. | Mean | S.E.M. | Mean | S.E.M. |
| PBS | 100.9 | 6.8 | — | — | — | — |
| DT-000812 | — | — | 91.9 | 9.7 | 56.2 | 5.5 |
| DT-001250 | — | — | 105.2 | 10.4 | 40.8 | 4.8 |
| DT-001251 | — | — | 117.2 | 12.5 | 51.5 | 5.4 |
| DT-001252 | — | — | 79.8 | 4.8 | 61.1 | 7.5 |
| DT-001253 | — | — | 88.3 | 10.6 | 53.2 | 3.5 |

Brachial Plexus

| | PBS | | 10 mg/kg | | 30 mg/kg | |
|---|---|---|---|---|---|---|
| | Mean | S.E.M. | Mean | S.E.M. | Mean | S.E.M. |
| PBS | 104.4 | 15.5 | — | — | — | — |
| DT-000812 | — | — | 79.3 | 9.7 | 49.1 | 5.6 |
| DT-001250 | — | — | 84.2 | 7.8 | 35.6 | 6.5 |
| DT-001251 | — | — | 85.8 | 9.2 | 49.3 | 5.1 |
| DT-001252 | — | — | 60.3 | 5.7 | 40.0 | 3.6 |
| DT-001253 | — | — | 61.1 | 5.0 | 30.1 | 3.8 |

TABLE 52
Human PMP22 mRNA 14 days following a single injection
of 10 mg/kg or 30 mg/kg of conjugated siRNA compound
Sciatic Nerve

| | PBS | | 10 mg/kg | | 30 mg/kg | |
|---|---|---|---|---|---|---|
| Treatment | Mean | S.E.M. | Mean | S.E.M. | Mean | S.E.M. |
| PBS | 101.3 | 7.2 | — | — | — | — |
| DT-000812 | — | — | 79.9 | 5.6 | 54.1 | 3.7 |
| DT-001254 | — | — | 82.0 | 6.8 | 40.0 | 3.4 |
| DT-001255 | — | — | 73.4 | 4.0 | 33.9 | 4.2 |
| DT-001257 | — | — | 67.7 | 5.8 | 28.8 | 5.8 |

Brachial Plexus

| | PBS | | 10 mg/kg | | 30 mg/kg | |
|---|---|---|---|---|---|---|
| | Mean | S.E.M. | Mean | S.E.M. | Mean | S.E.M. |
| PBS | 101.2 | 6.8 | — | — | — | — |
| DT-000812 | — | — | 79.5 | 6.9 | 53.9 | 3.6 |
| DT-001254 | — | — | 83.6 | 4.1 | 40.0 | 4.3 |
| DT-001255 | — | — | 73.7 | 8.3 | 29.4 | 5.0 |
| DT-001257 | — | — | 72.0 | 6.6 | 19.7 | 2.7 |

### Example 9: Evaluating Efficacy of Conjugated PMP22 siRNAs in a Mouse Model of CMT1A

C3-PMP22 mice are used as an experimental model of Charcot-Marie-Tooth disease type 1A (CMT1A). These transgenic mice express three to four copies of a wild-type human PMP22 gene, which leads to reduced numbers of myelinated fibers as early as three weeks of age. C3-PMP22 mice exhibit symptoms of neuromuscular impairment in the limbs similar to those observed in humans with CMT1A. Measurable functional endpoints in C3-PMP22 mice include, for example, motor nerve conduction velocity (MNCV), compound muscle action potential (CMAP), grip strength and beam walking.

The MNCV test is a non-invasive test that measures the velocity of a nerve signal. In this test, two electrodes are placed along a nerve, and the signal transduced between those electrodes is captured via a recording electrode placed at the neuromuscular junction. Defects in the myelin sheath in subjects with CMT1A cause a reduction in MNCV and a decrease in the amplitude of the transduced signal. These same findings are observed in C3-PMP22 mice.

CMAP is a quantitative measure of the amplitude of the electrical impulses that are transmitted to muscle. CMAP correlates with the number of muscle fibers that can be activated. In subjects with CMT1A, the CMAP of the nerve controlling contraction of the Anterior Tibialis muscle, a major muscle in the lower leg, correlates significantly with leg strength. These same findings are present in C3-PMP22 mice.

In the beam walking test, the dexterity of mice is observed as they walk along a horizontally suspended beam. Wild-type mice easily traverse the entire length of the beam. CMT1A mice, however, proceed more slowly and their paws may slip off the beam.

In the grip strength test, the mouse grasps a grid attached to a force transducer while an investigator gently pulls its tail. Grip strength is recorded as the force applied by the mouse in resisting the pulling motion. Relative to wild-type mice, grip strength of C3-PMP22 mice is reduced.

DT-000812 12-week efficacy study

The efficacy of DT-000812 was evaluated in C3-PMP22 mice. Groups of six mice each were treated with PBS, weekly doses of 10 mg/kg DT-000812 (on Day 1 and weekly thereafter for a total of 11 doses), and monthly doses of 30 mg/kg DT-000812 (on Day 1, Day 28, and Day 56 for a total of 3 doses). Wild-type mice treated with PBS were used as a control (WT-PBS). Motor nerve conduction velocity (MNCV) and compound muscle action potential (CMAP) were determined just prior to treatment and at 4, 8, and 12 weeks to establish a baseline value for each endpoint. At 12 weeks, mice were sacrificed, and sciatic and brachial plexus nerves were harvested for RNA extraction. Human PMP22 mRNA expression in C3-PMP22 mice was measured by quantitative RT-PCR. Additionally, the expression of the top 500 dysregulated genes in wild-type mice relative to C3-PMP22 was evaluated by RNAseq. Peripheral nerves were dissected and prepared for morphometric analysis according to routine methods (for example, Jolivalt, et al., 2016, Curr. Protoc. Mouse Biol., 6:223-255). Cross sections of nerve were processed into resin blocks which were cut into 0.5- to 1.3-μm thick sections, stained with p-phenylenediamine, and viewed by light microscopy. Axon diameters and myelin thickness were measured using a software-assisted manual approach in ImageJ/FIJI.

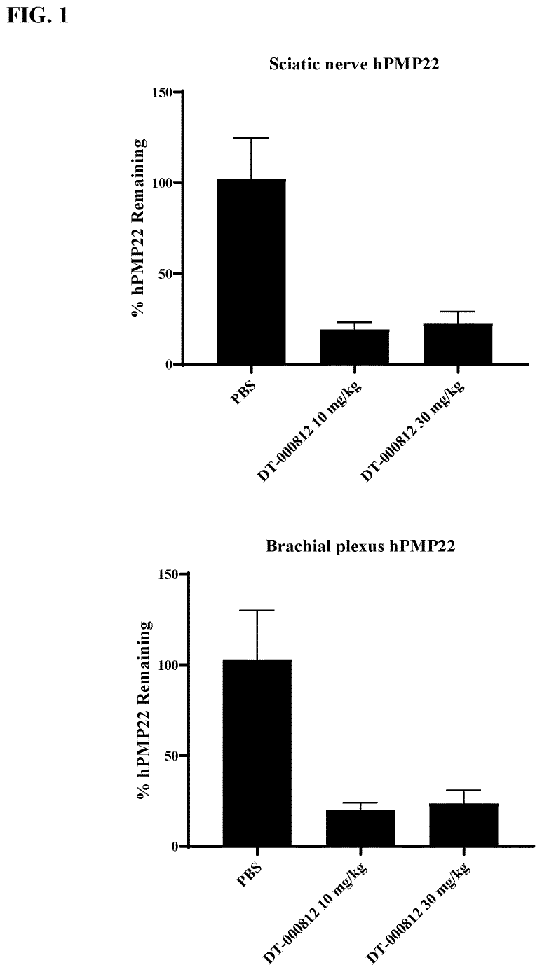

The average percent expression for human PMP22 mRNA was calculated for each treatment and is shown in Table 53 and FIG. 1. The expression of several myelin-specific mouse mRNAs was also measured by quantitative RT-PCR. The average percent expression for each of these mRNAs was calculated and is shown in Table 58.

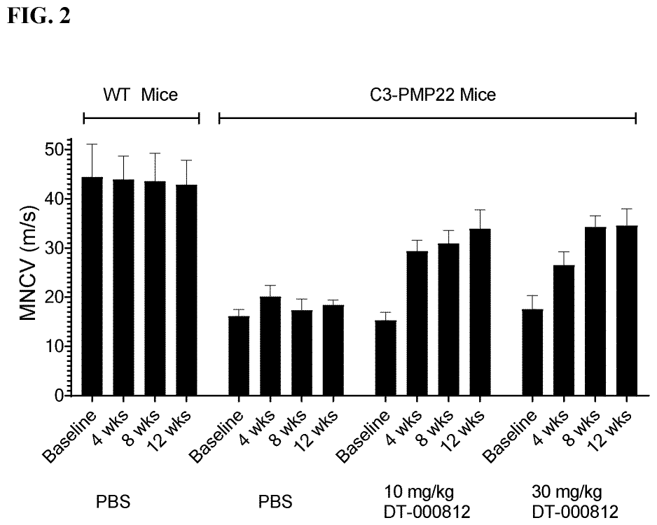

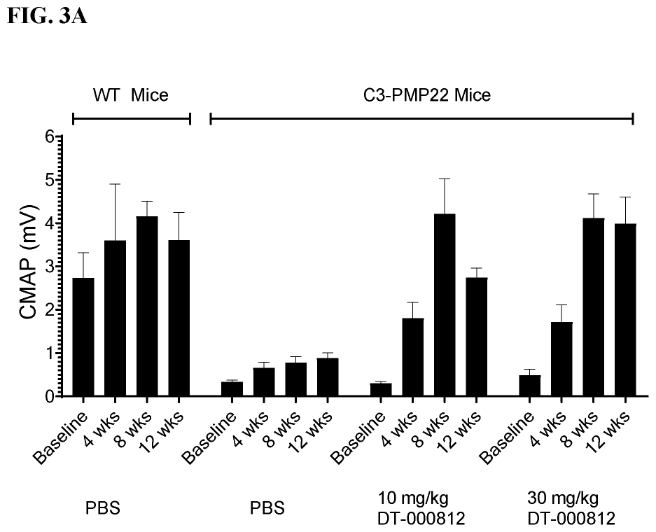

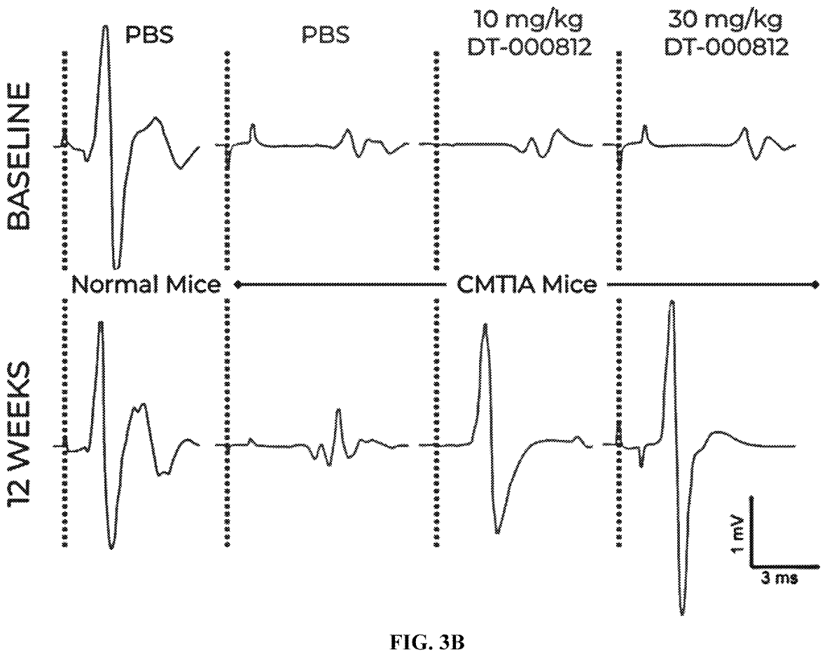

The average MNCV per treatment group are shown in Table 54 and FIG. 2. The average CMAP per treatment group are shown in Table 55 and FIG. 3. Grip strength and beam walking ability were measured at 12 weeks and are shown in Table 56.

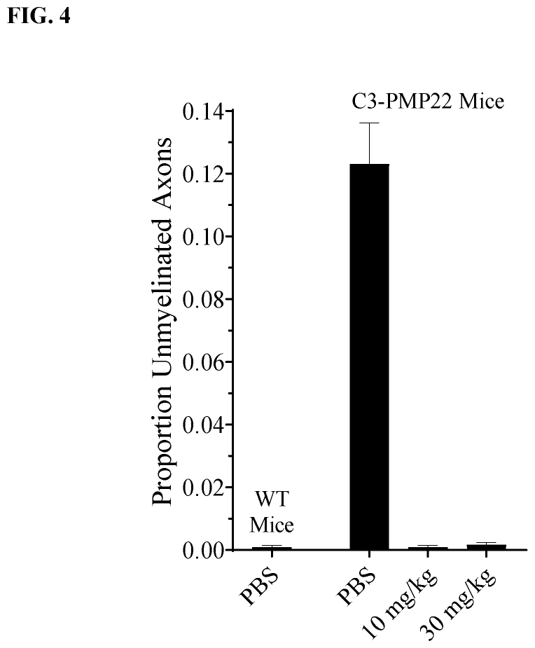

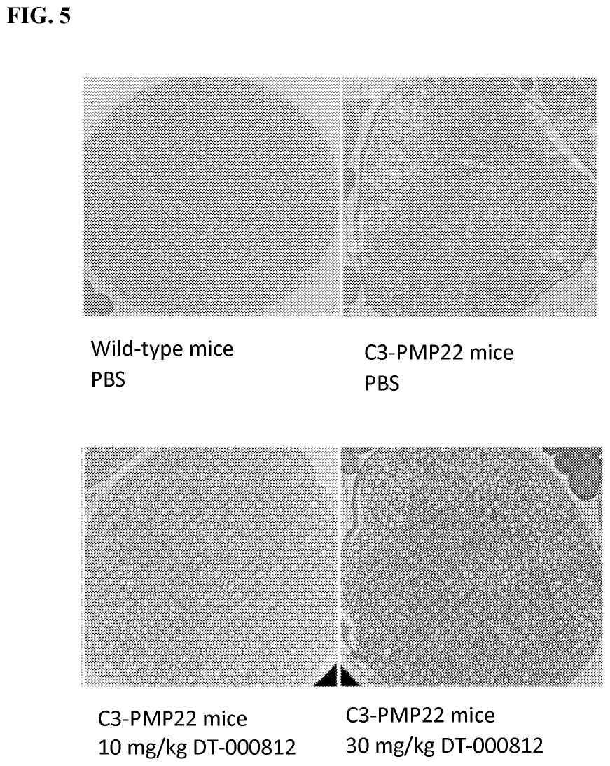

The mean proportion of unmyelinated axons in each treatment group is shown in Table 57 and FIG. 4. Representative sections of peripheral axon are shown in FIG. 5.

In each table, WT-PBS indicates wild-type mice treated with PBS; all other data were obtained in C3-PMP22 mice (PBS, 10 mg/kg DT-000812, and 30 mg/kg DT-000812).

TABLE 53
Human PMP22 mRNA 12 weeks following weekly
injections of 10 mg/kg or monthly injections
of 30 mg/kg of conjugated siRNA compound

| | |
|---|---|
| Sciatic | Brachial Plexus |

| Treatment | Mean | S.E.M. | Mean | S.E.M. |
|---|---|---|---|---|
| PBS | 101.9 | 9.3 | 103.0 | 11.0 |
| 10 mg/kg DT-000812 | 22.8 | 2.5 | 23.7 | 2.9 |
| 30 mg/kg DT-000812 | 19.2 | 1.6 | 19.9 | 1.7 |

TABLE 54
MNCV prior to and following weekly injections of 10 mg/kg or
monthly injections of 30 mg/kg of conjugated siRNA compound

| | Baseline | | 4 weeks | | 8 weeks | | 12 weeks | |
|---|---|---|---|---|---|---|---|---|
| | Mean | | Mean | | Mean | | Mean | |
| Treatment | m/s | S.E.M. | m/s | S.E.M. | m/s | S.E.M. | m/s | S.E.M. |
| WT- PBS | 44.4 | 16.3 | 43.9 | 11.6 | 43.6 | 13.9 | 42.8 | 12.2 |
| PBS | 16.2 | 3.4 | 20.1 | 5.5 | 17.4 | 5.6 | 18.4 | 2.5 |
| 10 mg/kg DT- | 15.3 | 3.9 | 29.4 | 5.3 | 30.9 | 6.4 | 33.9 | 9.3 |
| 000812 | | | | | | | | |
| 30 mg/kg DT- | 17.6 | 6.6 | 26.5 | 6.5 | 34.3 | 5.5 | 34.6 | 8.2 |
| 000812 | | | | | | | | |

TABLE 55
CMAP prior to and following weekly injections of 10 mg/kg or
monthly injections of 30 mg/kg of conjugated siRNA compound

| | Baseline | | 4 weeks | | 8 weeks | | 12 weeks | |
|---|---|---|---|---|---|---|---|---|
| Treatment | Mean | S.E.M. | Mean | S.E.M. | Mean | S.E.M. | Mean | S.E.M. |
| WT- PBS | 2.7 | 0.6 | 3.6 | 1.3 | 4.2 | 0.3 | 3.6 | 0.6 |
| PBS | 0.3 | 0.0 | 0.7 | 0.1 | 0.8 | 0.1 | 0.9 | 0.1 |
| 10 mg/kg DT- | 0.3 | 0.1 | 1.8 | 0.4 | 4.2 | 0.8 | 2.8 | 0.2 |
| 000812 | | | | | | | | |
| 30 mg/kg DT- | 0.5 | 0.1 | 1.7 | 0.4 | 4.1 | 0.6 | 4.0 | 0.6 |
| 000812 | | | | | | | | |

TABLE 56
Quantiation of myelination of peripheral nerves 12 weeks
following weekly injections of 10 mg/kg or monthly
injections of 30 mg/kg of conjugated siRNA compound

Proportion of
Unmyleinated Axons

| Treatment | Mean | S.E.M. |
|---|---|---|
| WT-PBS | 0.0009 | 0.0005 |
| PBS | 0.1231 | 0.0131 |
| 10 mg/kg DT-000812 | 0.0010 | 0.0005 |
| 30 mg/kg DT-000812 | 0.0018 | 0.0007 |

TABLE 57
Grip strength and beam walking ability prior to and
following weekly injections of 10 mg/kg or monthly
injections of 30 mg/kg of conjugated siRNA compound

| | Grip Strength | | Beam Walking | | Beam Walking | |
|---|---|---|---|---|---|---|
| | (g) | | (Latency) | | (Slips) | |
| Treatment | Mean | S.E.M. | Mean | S.E.M. | Mean | S.E.M. |
| WT- PBS | 197.7 | 17.2 | 12.1 | 5.3 | 0.5 | 0.3 |
| PBS | 126.7 | 2.5 | 25.8 | 3.0 | 22.8 | 4.9 |
| 10 mg/kg DT-000812 | 203.2 | 8.4 | 14.6 | 2.0 | 2.2 | 1.0 |
| 30 mg/kg DT-000812 | 221.4 | 10.3 | 13.8 | 1.6 | 1.3 | 1.0 |

TABLE 58
Myelin-specific mRNA expression following weekly
injections of 10 mg/kg or monthly injections
of 30 mg/kg of conjugated siRNA compound

| Sciatic | | Brachial Plexus | |
|---|---|---|---|
| Mean | S.E.M. | Mean | S.E.M. |

MPZ expression

| | | | | |
|---|---|---|---|---|
| WT- PBS | 101.6 | 7.8 | 104.7 | 14.0 |
| PBS | 107.4 | 7.2 | 131.8 | 13.1 |
| 10 mg/kg DT-000812 | 123.8 | 15.6 | 146.9 | 14.6 |
| 30 mg/kg DT-000812 | 107.4 | 5.9 | 132.5 | 10.4 |

Pou3F1 expression

| | | | | |
|---|---|---|---|---|
| WT- PBS | 102.5 | 9.7 | 139.0 | 37.7 |
| PBS | 1221.0 | 147.2 | 1722.0 | 249.5 |
| 10 mg/kg DT-000812 | 195.1 | 16.7 | 556.5 | 92.6 |
| 30 mg/kg DT-000812 | 180.8 | 21.0 | 295.6 | 50.5 |

CXCL14 expression

| | | | | |
|---|---|---|---|---|
| WT- PBS | 114.1 | 27.7 | 132.9 | 48.7 |
| PBS | 288.4 | 27.5 | 430.3 | 30.0 |
| 10 mg/kg DT-000812 | 87.1 | 7.5 | 81.7 | 26.1 |
| 30 mg/kg DT-000812 | 117.4 | 15.4 | 118.2 | 24.8 |

NGFR expression

| | | | | |
|---|---|---|---|---|
| WT- PBS | 104.5 | 13.5 | 117.8 | 24.8 |
| PBS | 602.4 | 65.3 | 1115.0 | 209.6 |
| 10 mg/kg DT-000812 | 185.6 | 26.1 | 324.1 | 52.8 |
| 30 mg/kg DT-000812 | 232.7 | 41.8 | 399.1 | 84.3 |

Sox4 expression

| | | | | |
|---|---|---|---|---|
| WT- PBS | 106.6 | 15.4 | 116.1 | 23.4 |
| PBS | 423.1 | 62.9 | 323.0 | 33.7 |
| 10 mg/kg DT-000812 | 124.9 | 9.5 | 213.8 | 40.5 |
| 30 mg/kg DT-000812 | 180.6 | 26.2 | 162.4 | 15.4 |

CSRP2 expression

| | | | | |
|---|---|---|---|---|
| WT- PBS | 111.6 | 23.5 | 106.3 | 16.2 |
| PBS | 594.3 | 35.2 | 207.7 | 24.5 |
| 10 mg/kg DT-000812 | 165.9 | 40.2 | 106.1 | 10.0 |
| 30 mg/kg DT-000812 | 138.9 | 16.3 | 151.6 | 18.8 |

CUEDC2 expression

| | | | | |
|---|---|---|---|---|
| WT- PBS | 102.0 | 9.3 | 103.3 | 12.0 |
| PBS | 358.2 | 23.3 | 249.1 | 23.5 |
| 10 mg/kg DT-000812 | 150.0 | 22.4 | 132.5 | 14.0 |
| 30 mg/kg DT-000812 | 141.3 | 14.4 | 121.0 | 7.2 |

OLFML2A expression

| | | | | |
|---|---|---|---|---|
| WT- PBS | 103.9 | 12.8 | 110.2 | 16.8 |
| PBS | 229.4 | 13.7 | 294.6 | 47.0 |
| 10 mg/kg DT-000812 | 122.6 | 24.5 | 174.0 | 14.4 |
| 30 mg/kg DT-000812 | 141.6 | 21.7 | 197.8 | 22.9 |

SERINC5 expression

| | | | | |
|---|---|---|---|---|
| WT- PBS | 103.9 | 12.8 | 110.2 | 16.8 |
| PBS | 229.4 | 13.7 | 294.6 | 47.0 |
| 10 mg/kg DT-000812 | 122.6 | 24.5 | 174.0 | 14.4 |
| 30 mg/kg DT-000812 | 141.6 | 21.7 | 197.8 | 22.9 |

As illustrated by the above data, substantial improvements in multiple endpoints associated with CMT1A were observed.

Treatment of C3-PMP22 mice with DT-000812 resulted in a reduction in human PMP22 mRNA in both the sciatic and brachial plexus nerves (Table 53 and FIG. 1).

The MNCV tests revealed an improvement in the efficiency of motor nerve conduction (Table 54 and FIG. 2). Additionally, histological analysis revealed that, whereas unmyelinated axons were common in sciatic nerve sections from C3-PMP22 mice, neither DT-000812 treatment group exhibited substantial numbers of large unmyelinated axons (Table 56, FIG. 4, and FIG. 5). Thus, the improvement in MNCV is likely due to an increase in the number of myelinated axons in C3-PMP22 mice. The combination of the functional recovery of MNCV and increase in myelinated neurons following treatment with DT-000812 is consistent with a reversal of demyelination, the primary physiological defect of CMT1A.

In wild-type mice, CMAP consisted of a strong electrical polarization signal, followed by a depolarization signal. In C3-PMP22 mice, both signals were muted and difficult to distinguish from background electrical impulses. In contrast, treatment with DT-000812 restored the shape and amplitude of CMAPs in C3-PMP22 mice (FIG. 3B).

In the beam walking test, wild-type mice easily traversed the entire length of the beam. In contrast, PBS-treated C3-PMP22 mice proceeded much more slowly, and their hind paws repeatedly slipped off the beam and on average required twice the amount of time to travel the same distance as wild-type mice. After twelve weeks of treatment of C3-PMP22 mice with DT-000812, the speed at which the mice traversed the beam was close to that of wild-type mice. Additionally, the number of slips relative to PBS-treated C3-PMP22 mice was reduced.

The grip strength of C3-PMP22 mice mice treated with PBS was markedly reduced relative to wild-type mice. Treatment with DT-000812 over a 12-week period increased forelimb grip strength to a level equivalent of wild-type mice. Furthermore, DT-000812 treatment over this same period led to increases in the mass of several peripheral muscles (quadricep and gastrocnemius) relative to untreated C3-PMP22 mice.

Measurement of nine genes essential for Schwann cell function illustrated that DT-000812 restored gene expression of these genes in the sciatic and brachial plexus nerves to the levels observed in wild-type mice. Additionally, RNAseq analysis revealed that the large majority of genes dysregulated in C3-PMP22 mice were restored toward wild-type levels of mRNA expression following treatment with DT-000812 at both the 10 mg/kg and 30 mg/kg doses.

Taken, these data demonstrate that inhibition of PMP22 with DT-000812 in C3-PMP22 mice, a model for CMT1A in human subjects, leads to substantial improvements in multiple phenotypes associated with CMT1A.

DT-000812, DT-001246, DT-001247 28-day efficacy study

The efficacies DT-001246 and DT-001247 were evaluated, and compared to DT-000812, in C3-PMP22 mice. Groups of eight mice each were treated with PBS and a single dose of 30 mg/kg of each compound on Day 0 of the study. Motor nerve conduction velocity (MNCV) and compound muscle action potential (CMAP) were determined just prior to treatment (Baseline; Day -1) and at Day 27. At Day 28, mice were sacrificed, and sciatic and brachial plexus nerve tissues were harvested for RNA extraction. Human PMP22 mRNA expression was measured by quantitative RT-PCR.

The average percent expression for human PMP22 mRNA was calculated for each treatment and is shown in Table 59. MNCV and CMAP are shown in Table 60. The expression of several myelin-specific mouse mRNAs was also measured by quantitative RT-PCR. The average percent expression for each of these mRNAs was calculated and is shown in Table 61.

TABLE 59
Human PMP22 mRNA 28 days following a single
dose of 30 mg/kg conjugated PMP22 siRNAs

| | |
|---|---|
| Sciatic | Brachial Plexus |

| Treatment | Mean | S.E.M. | Mean | S.E.M. |
|---|---|---|---|---|
| PBS | 99.2 | 5.5 | 107.6 | 12.0 |
| DT-000812 | 49.6 | 3.2 | 49.8 | 8.0 |
| DT-001246 | 50.8 | 3.9 | 40.7 | 6.0 |
| DT-001247 | 62.3 | 3.1 | 50.8 | 3.2 |

TABLE 60
MNCV and CMAP at Baseline and 27 days following
a single dose of 30 mg/kg conjugated PMP22 siRNAs

| | |
|---|---|
| Baseline | 28 days |

| | | | | |
|---|---|---|---|---|
| Treatment | Mean | S.E.M. | Mean | S.E.M. |

MNCV

| | | | | |
|---|---|---|---|---|
| PBS | 11.6 | 3.0 | 16.2 | 1.0 |
| DT-000812 | 18.3 | 2.4 | 27.7 | 1.0 |
| DT-001246 | 16.0 | 2.2 | 28.8 | 1.5 |
| DT-001247 | 18.7 | 1.7 | 26.8 | 2.9 |

CMAP

| | | | | |
|---|---|---|---|---|
| PBS | 0.8 | 0.1 | 1.5 | 0.3 |
| DT-000812 | 0.9 | 0.1 | 3.7 | 0.6 |
| DT-001246 | 1.5 | 0.1 | 3.1 | 0.5 |
| DT-001247 | 1.2 | 0.1 | 2.9 | 0.5 |

TABLE 61
Mouse myelin-specifc mRNA expression 28 days following
a single dose of 30 mg/kg conjugated PMP22 siRNAs

| | |
|---|---|
| Sciatic | Brachial |

| | | | | |
|---|---|---|---|---|
| Treatment | Mean | S.E.M. | Mean | S.E.M. |

MPZ expression

| | | | | |
|---|---|---|---|---|
| PBS | 106.4 | 8.0 | 115.3 | 13.9 |
| DT-000812 | 165.1 | 19.8 | 199.8 | 26.1 |
| DT-001246 | 217.0 | 21.0 | 178.5 | 29.0 |
| DT-001247 | 198.1 | 16.3 | 201.7 | 17.5 |

Pou3F1 expression

| | | | | |
|---|---|---|---|---|
| PBS | 104.4 | 16.8 | 111.0 | 20.3 |
| DT-000812 | 30.5 | 1.6 | 31.8 | 5.6 |
| DT-001246 | 41.1 | 2.8 | 46.1 | 8.7 |
| DT-001247 | 42.6 | 2.8 | 67.0 | 9.0 |

CXCL14 expression

| | | | | |
|---|---|---|---|---|
| PBS | 103 | 8.382 | 97.6 | 11.8 |
| DT-000812 | 43.28 | 2.859 | 23.2 | 1.9 |
| DT-001246 | 43.26 | 3.992 | 20.4 | 3.5 |
| DT-001247 | 39.37 | 2.97 | 20.2 | 1.9 |

NGFR expression

| | | | | |
|---|---|---|---|---|
| PBS | 99.2 | 10.8 | 100.5 | 9.3 |
| DT-000812 | 55.4 | 3.9 | 68.1 | 7.6 |
| DT-001246 | 68.3 | 6.7 | 56.0 | 8.6 |
| DT-001247 | 69.2 | 5.3 | 66.3 | 6.5 |

CSRP2 expression

| | | | | |
|---|---|---|---|---|
| PBS | 110.7 | 12.7 | 94.6 | 15.3 |
| DT-000812 | 44.5 | 3.1 | 40.9 | 5.7 |
| DT-001246 | 45.3 | 2.1 | 28.3 | 4.8 |
| DT-001247 | 45.6 | 4.1 | 38.7 | 6.7 |

CUEDC2 expression

| | | | | |
|---|---|---|---|---|
| PBS | 100.8 | 9.4 | 105.9 | 12.1 |
| DT-000812 | 53.7 | 2.3 | 62.8 | 5.6 |
| DT-001246 | 64.4 | 4.2 | 60.9 | 8.3 |
| DT-001247 | 60.8 | 3.7 | 64.2 | 4.1 |

OLFML2A expression

| | | | | |
|---|---|---|---|---|
| PBS | 101.3 | 11.0 | 97.9 | 6.6 |
| DT-000812 | 66.6 | 4.4 | 54.7 | 6.3 |
| DT-001246 | 77.5 | 6.3 | 57.3 | 8.3 |
| DT-001247 | 80.2 | 2.4 | 70.7 | 8.0 |

### DT-00812, DT-001246, DT-001247 60-Day Efficacy Study

DT-000812, DT-001246, and DT-001247 were evaluated in a 60-day efficacy study in C3-PMP22 mice. Groups of eight mice each were treated with PBS and a single dose of 30 mg/kg of each compound on Day 0 of the study. Motor nerve conduction velocity (MNCV) and compound muscle action potential (CMAP) were determined just prior to treatment (Baseline; Day -1) and at Day 59. At Day 60, mice were sacrificed, and sciatic and brachial plexus nerve tissues were harvested for RNA extraction. Human PMP22 mRNA expression was measured by quantitative RT-PCR. The expression of several myelin-specific mouse mRNAs was also measured by quantitative RT-PCR.

The average percent expression for human PMP22 mRNA was calculated for each treatment and is shown in Table 62. MNCV and CMAP are shown in Table 63. The average percent expression for the myelin-specific mRNAs was calculated and is shown in Table 64.

TABLE 62
Human PMP22 mRNA 60 days following a single
dose of 30 mg/kg conjugated PMP22 siRNAs

| | |
|---|---|
| Sciatic | Brachial Plexus |

| Treatment | Mean | S.E.M. | Mean | S.E.M. |
|---|---|---|---|---|
| PBS | 102.3 | 8.0 | 102.3 | 8.0 |
| DT-000812 | 46.1 | 4.8 | 46.1 | 4.8 |
| DT-001246 | 58.3 | 5.5 | 58.3 | 5.5 |
| DT-001247 | 55.2 | 2.5 | 55.2 | 2.5 |

TABLE 63
MNCV and CMAP at Baseline and Days 28 and 59 following
a single dose of 30 mg/kg conjugated PMP22 siRNAs

| | Baseline | | 28 days | | 59 days | |
|---|---|---|---|---|---|---|
| Treatment | Mean | S.E.M. | Mean | S.E.M. | Mean | S.E.M. |
| | MNCV | | | | | |
| PBS | 30.5 | 2.2 | 22.9 | 1.6 | 36.0 | 4.3 |
| DT-000812 | 27.0 | 2.0 | 37.2 | 3.2 | 40.5 | 3.2 |
| DT-001246 | 29.2 | 2.1 | 27.9 | 3.1 | 36.6 | 3.8 |
| DT-001247 | 29.8 | 1.2 | 27.2 | 3.6 | 40.1 | 4.6 |
| | CMAP | | | | | |
| PBS | 1.2 | 0.2 | 1.1 | 0.2 | 0.8 | 0.1 |
| DT-000812 | 1.2 | 0.2 | 2.5 | 0.5 | 3.6 | 0.8 |
| DT-001246 | 1.5 | 0.2 | 2.7 | 0.6 | 2.1 | 0.5 |
| DT-001247 | 1.9 | 0.3 | 2.8 | 0.7 | 3.2 | 0.4 |

TABLE 64
Myelin-specific mRNA expression 60 days following
a single dose of 30 mg/kg conjugated PMP22 siRNAs

| | |
|---|---|
| Sciatic | Brachial |

| | | | | |
|---|---|---|---|---|
| Treatment | Mean | S.E.M. | Mean | S.E.M. |

MPZ expression

| | | | | |
|---|---|---|---|---|
| PBS | 101.8 | 7.4 | 101.0 | 5.5 |
| DT-000812 | 176.6 | 6.8 | 164.5 | 7.6 |
| DT-001246 | 179.8 | 13.3 | 161.6 | 8.9 |
| DT-001247 | 187.2 | 7.1 | 183.6 | 17.8 |

Pou3F1 expression

| | | | | |
|---|---|---|---|---|
| PBS | 103.4 | 10.0 | 103.1 | 8.8 |
| DT-000812 | 45.1 | 4.9 | 51.8 | 9.1 |
| DT-001246 | 47.3 | 5.8 | 51.9 | 4.4 |
| DT-001247 | 40.8 | 3.1 | 53.9 | 5.5 |

CXCL14 expression

| | | | | |
|---|---|---|---|---|
| PBS | 100.9 | 5.4 | 106.5 | 13.9 |
| DT-000812 | 51.6 | 7.5 | 29.2 | 4.8 |
| DT-001246 | 57.2 | 5.5 | 29.2 | 3.9 |
| DT-001247 | 42.0 | 4.6 | 28.5 | 3.0 |

NGFR expression

| | | | | |
|---|---|---|---|---|
| PBS | 105.6 | 11.9 | 102.6 | 8.5 |
| DT-000812 | 67.8 | 5.3 | 111.7 | 44.4 |
| DT-001246 | 74.8 | 6.6 | 100.4 | 31.5 |
| DT-001247 | 72.4 | 4.9 | 67.5 | 6.0 |

CSRP2 expression

| | | | | |
|---|---|---|---|---|
| PBS | 101.8 | 7.0 | 108.2 | 14.2 |
| DT-000812 | 46.7 | 6.1 | 42.3 | 7.2 |
| DT-001246 | 41.9 | 4.0 | 50.0 | 6.0 |
| DT-001247 | 40.8 | 4.2 | 42.1 | 5.4 |

CUEDC2 expression

| | | | | |
|---|---|---|---|---|
| PBS | 102.1 | 7.4 | 101.1 | 5.9 |
| DT-000812 | 61.7 | 3.5 | 59.8 | 4.4 |
| DT-001246 | 60.7 | 2.9 | 54.2 | 3.2 |
| DT-001247 | 60.0 | 4.1 | 57.3 | 5.3 |

OLFML2A expression

| | | | | |
|---|---|---|---|---|
| PBS | 101.4 | 6.6 | 106.0 | 15.1 |
| DT-000812 | 76.0 | 7.3 | 77.4 | 5.6 |
| DT-001246 | 68.3 | 7.0 | 65.4 | 10.0 |
| DT-001247 | 74.9 | 5.4 | 71.3 | 10.8 |

DT-000812, DT-001250, DT-001251, DT-001252, DT-001253 28-day efficacy study

The efficacies of DT-001250, DT-001251, DT-001252, and DT-001253 were evaluated, and compared to DT-000812, in C3-PMP22 mice. Groups of eight mice each were treated with PBS and a single dose of 30 mg/kg of each compound on Day 0 of the study. Motor nerve conduction velocity (MNCV) and compound muscle action potential (CMAP) were determined just prior to treatment (Baseline; Day -1) and at Day 27. At Day 28, mice were sacrificed, and sciatic and brachial plexus nerve tissues were harvested for RNA extraction. Human PMP22 mRNA expression was measured by quantitative RT-PCR.

The average percent expression for human PMP22 mRNA was calculated for each treatment and is shown in Table 65. MNCV and CMAP are shown in Table 66. The expression of several myelin-specific mouse mRNAs was also measured by quantitative RT-PCR. The average percent expression for each of these mRNAs was calculated and is shown in Table 67.

TABLE 65
Human PMP22 mRNA 28 days following a single
dose of 30 mg/kg conjugated PMP22 siRNAs

| | |
|---|---|
| Sciatic | Brachial Plexus |

| Treatment | Mean | S.E.M. | Mean | S.E.M. |
|---|---|---|---|---|
| PBS | 101.4 | 6.3 | 100.6 | 3.9 |
| DT-000812 | 63.7 | 6.0 | 64.6 | 6.1 |
| DT-001250 | 38.9 | 3.1 | 40.4 | 4.2 |
| DT-001251 | 25.2 | 2.9 | 28.0 | 5.1 |
| DT-001252 | 47.0 | 4.5 | 49.4 | 5.2 |
| DT-001253 | 26.0 | 2.8 | 32.1 | 4.0 |

TABLE 66
MNCV and CMAP at Baseline and 28 days following
a single dose of 30 mg/kg conjugated PMP22 siRNAs

| | |
|---|---|
| Baseline | 28 days |

| | | | | |
|---|---|---|---|---|
| Treatment | Mean | S.E.M. | Mean | S.E.M. |

MNCV

| | | | | |
|---|---|---|---|---|
| PBS | 23.1 | 0.9 | 20.7 | 1.5 |
| DT-000812 | 29.2 | 2.8 | 32.4 | 1.9 |
| DT-001250 | 23.5 | 1.6 | 27.7 | 2.0 |
| DT-001251 | 21.2 | 1.7 | 29.0 | 2.1 |
| DT-001252 | 26.2 | 2.4 | 35.1 | 2.1 |
| DT-001253 | 31.0 | 8.1 | 29.6 | 2.7 |

CMAP

| | | | | |
|---|---|---|---|---|
| PBS | 1.0 | 0.2 | 1.5 | 0.3 |
| DT-000812 | 1.7 | 0.2 | 3.8 | 0.7 |
| DT-001250 | 1.5 | 0.2 | 2.9 | 0.7 |
| DT-001251 | 1.2 | 0.2 | 3.8 | 0.8 |
| DT-001252 | 1.7 | 0.2 | 2.7 | 0.5 |
| DT-001253 | 1.3 | 0.3 | 1.6 | 0.3 |

TABLE 67
Myelin-specific mRNA expression 28 days following
a single dose of 30 mg/kg conjugated PMP22 siRNAs

| | |
|---|---|
| Sciatic | Brachial |

| | | | | |
|---|---|---|---|---|
| Treatment | Mean | S.E.M. | Mean | S.E.M. |

MPZ expression

| | | | | |
|---|---|---|---|---|
| PBS | 101.4 | 6.4 | 100.9 | 5.1 |
| DT-000812 | 165.9 | 9.6 | 181.6 | 13.9 |
| DT-001250 | 170.1 | 8.5 | 193.8 | 15.4 |
| DT-001251 | 165.3 | 12.7 | 211.9 | 31.7 |
| DT-001252 | 184.5 | 11.6 | 194.5 | 21.4 |
| DT-001253 | 146.8 | 8.6 | 212.0 | 15.3 |

Pou3F1 expression

| | | | | |
|---|---|---|---|---|
| PBS | 101.4 | 6.2 | 104.6 | 12.5 |
| DT-000812 | 45.2 | 7.9 | 53.1 | 8.7 |
| DT-001250 | 28.7 | 2.1 | 42.7 | 6.2 |
| DT-001251 | 30.1 | 3.3 | 42.6 | 8.8 |
| DT-001252 | 39.2 | 3.1 | 55.8 | 5.5 |
| DT-001253 | 27.0 | 3.3 | 42.6 | 4.9 |

CXCL14 expression

| | | | | |
|---|---|---|---|---|
| PBS | 102.3 | 7.8 | 103.8 | 11.1 |
| DT-000812 | 57.0 | 4.6 | 41.8 | 6.1 |
| DT-001250 | 50.7 | 3.3 | 26.3 | 4.1 |
| DT-001251 | 64.0 | 16.1 | 58.3 | 38.7 |
| DT-001252 | 49.6 | 8.1 | 34.1 | 5.5 |
| DT-001253 | 57.7 | 7.1 | 23.2 | 2.7 |

NGFR expression

| | | | | |
|---|---|---|---|---|
| PBS | 102.5 | 8.7 | 104.4 | 11.5 |
| DT-000812 | 58.4 | 6.3 | 103.5 | 31.3 |
| DT-001250 | 46.5 | 4.5 | 102.8 | 44.5 |
| DT-001251 | 54.4 | 5.8 | 78.0 | 18.2 |
| DT-001252 | 83.8 | 5.9 | 171.2 | 53.3 |
| DT-001253 | 52.8 | 4.1 | 81.1 | 8.5 |

CSRP2 expression

| | | | | |
|---|---|---|---|---|
| PBS | 104.0 | 9.7 | 106.1 | 15.6 |
| DT-000812 | 55.1 | 7.7 | 41.9 | 7.1 |
| DT-001250 | 47.8 | 2.0 | 47.3 | 9.3 |
| DT-001251 | 41.5 | 5.0 | 49.8 | 11.2 |
| DT-001252 | 45.9 | 4.4 | 61.7 | 10.2 |
| DT-001253 | 37.3 | 3.0 | 40.8 | 6.0 |

CUEDC2 expression

| | | | | |
|---|---|---|---|---|
| PBS | 101.8 | 7.1 | 104.4 | 11.3 |
| DT-000812 | 66.7 | 5.7 | 79.3 | 7.4 |
| DT-001250 | 56.0 | 3.3 | 86.6 | 9.9 |
| DT-001251 | 48.3 | 2.5 | 77.3 | 8.9 |
| DT-001252 | 57.6 | 1.8 | 96.3 | 7.7 |
| DT-001253 | 41.2 | 2.5 | 76.5 | 4.5 |

OLFML2A expression

| | | | | |
|---|---|---|---|---|
| PBS | 105.0 | 14.6 | 103.9 | 10.3 |
| DT-000812 | 102.5 | 10.7 | 76.3 | 11.0 |
| DT-001250 | 78.1 | 6.0 | 54.3 | 8.9 |
| DT-001251 | 65.2 | 7.3 | 56.3 | 6.8 |
| DT-001252 | 78.6 | 9.3 | 77.5 | 8.3 |
| DT-001253 | 59.0 | 4.3 | 64.0 | 7.9 |

### DT-00812, DT-001250, DT-001251, DT-001252, DT-001253 60-Day Efficacy Study

DT-000812, DT-001250, DT-001251, DT-001252, and DT-001253 were evaluated in a 60-day efficacy study in C3-PMP22 mice. Groups of eight mice each were treated with PBS and a single dose of 30 mg/kg of each compound on Day 0 of the study. Motor nerve conduction velocity (MNCV) and compound muscle action potential (CMAP) were determined just prior to treatment (Baseline; Day -1), at Day 28 and at Day 59. At Day 60, mice were sacrificed, and sciatic and brachial plexus nerve tissues were harvested for RNA extraction. Human PMP22 mRNA expression was measured by quantitative RT-PCR. The expression of several myelin-specific mouse mRNAs was also measured by quantitative RT-PCR.

The average percent expression for human PMP22 mRNA was calculated for each treatment and is shown in Table 68. MNCV and CMAP are shown in Table 69. The average percent expression for the myelin-specific mRNAs was calculated and is shown in Table 70.

TABLE 68
Human PMP22 mRNA 60 days following a single
dose of 30 mg/kg conjugated PMP22 siRNAs

| | |
|---|---|
| Sciatic | Brachial Plexus |

| Treatment | Mean | S.E.M. | Mean | S.E.M. |
|---|---|---|---|---|
| PBS | 101.2 | 5.9 | 100.8 | 4.6 |
| DT-000812 | 80.4 | 5.4 | 59.0 | 4.9 |
| DT-001250 | 50.8 | 8.4 | 47.1 | 4.6 |
| DT-001251 | 73.0 | 6.1 | 56.5 | 2.9 |
| DT-001252 | 33.8 | 1.2 | 23.9 | 2.5 |
| DT-001253 | 35.9 | 2.9 | 28.9 | 3.0 |

TABLE 69
MNCV and CMAP at Baseline and Days 28 and 59 following
a single dose of 30 mg/kg conjugated PMP22 siRNAs

| | Baseline | | 28 days | | 59 days | |
|---|---|---|---|---|---|---|
| Treatment | Mean | S.E.M. | Mean | S.E.M. | Mean | S.E.M. |
| | MNCV | | | | | |
| PBS | 30.5 | 3.4 | 24.4 | 1.1 | 22.6 | 2.6 |
| DT-000812 | 23.1 | 2.2 | 33.2 | 2.6 | 50.2 | 11.9 |
| DT-001250 | 27.0 | 2.8 | 33.4 | 3.3 | 46.3 | 6.3 |
| DT-001251 | 26.0 | 2.8 | 27.7 | 1.7 | 32.0 | 1.8 |
| DT-001252 | 30.7 | 1.2 | 34.2 | 5.4 | 39.5 | 3.1 |
| DT-001253 | 24.1 | 2.6 | 32.7 | 4.2 | 51.2 | 11.0 |
| | CMAP | | | | | |
| PBS | 1.2 | 0.3 | 1.3 | 0.3 | 1.4 | 0.3 |
| DT-000812 | 0.8 | 0.1 | 3.4 | 1.0 | 2.3 | 0.5 |
| DT-001250 | 1.5 | 0.3 | 2.4 | 0.4 | 1.8 | 0.2 |
| DT-001251 | 1.3 | 0.3 | 2.8 | 0.4 | 1.5 | 0.3 |
| DT-001252 | 1.2 | 0.2 | 5.5 | 0.8 | 2.9 | 0.7 |
| DT-001253 | 1.3 | 0.2 | 3.0 | 0.6 | 3.4 | 0.5 |

TABLE 70
Myelin-specific mRNA expression 60 days following
a single dose of 30 mg/kg conjugated PMP22 siRNAs

| | |
|---|---|
| Sciatic | Brachial |

| | | | | |
|---|---|---|---|---|
| Treatment | Mean | S.E.M. | Mean | S.E.M. |

MPZ expression

| | | | | |
|---|---|---|---|---|
| PBS | 100.7 | 4.6 | 100.5 | 3.9 |
| DT-000812 | 144.1 | 8.3 | 119.8 | 5.3 |
| DT-001250 | 156.1 | 9.6 | 132.8 | 3.9 |
| DT-001251 | 157.0 | 11.0 | 130.8 | 6.7 |
| DT-001252 | 174.8 | 7.1 | 147.2 | 6.3 |
| DT-001253 | 178.4 | 13.1 | 128.7 | 7.9 |

Pou3F1 expression

| | | | | |
|---|---|---|---|---|
| PBS | 103.8 | 11.0 | 103.8 | 11.2 |
| DT-000812 | 48.0 | 3.1 | 38.7 | 4.4 |
| DT-001250 | 45.5 | 4.2 | 46.5 | 5.7 |
| DT-001251 | 55.4 | 5.9 | 51.1 | 5.0 |
| DT-001252 | 42.1 | 3.9 | 37.5 | 5.5 |
| DT-001253 | 47.5 | 5.1 | 38.0 | 3.1 |

CXCL14 expression

| | | | | |
|---|---|---|---|---|
| PBS | 100.5 | 3.9 | 103.0 | 8.8 |
| DT-000812 | 61.3 | 7.2 | 28.5 | 3.4 |
| DT-001250 | 51.6 | 3.6 | 22.4 | 4.0 |
| DT-001251 | 50.4 | 4.8 | 25.6 | 1.7 |
| DT-001252 | 41.5 | 2.7 | 13.2 | 2.4 |
| DT-001253 | 39.2 | 4.3 | 13.5 | 2.8 |

NGFR expression

| | | | | |
|---|---|---|---|---|
| PBS | 102.7 | 8.6 | 102.2 | 8.7 |
| DT-000812 | 68.4 | 5.7 | 80.9 | 15.1 |
| DT-001250 | 69.9 | 6.6 | 83.2 | 15.3 |
| DT-001251 | 78.7 | 11.2 | 95.1 | 14.3 |
| DT-001252 | 61.9 | 3.1 | 56.9 | 7.1 |
| DT-001253 | 65.7 | 10.6 | 90.9 | 30.7 |

CSRP2 expression

| | | | | |
|---|---|---|---|---|
| PBS | 101.4 | 6.1 | 104.5 | 11.8 |
| DT-000812 | 51.4 | 3.6 | 39.4 | 3.0 |
| DT-001250 | 42.0 | 4.6 | 37.3 | 5.2 |
| DT-001251 | 53.2 | 4.6 | 37.2 | 6.2 |
| DT-001252 | 43.7 | 3.0 | 34.0 | 6.7 |
| DT-001253 | 44.1 | 2.4 | 26.5 | 4.0 |

CUEDC2 expression

| | | | | |
|---|---|---|---|---|
| PBS | 102.2 | 8.1 | 102.9 | 9.3 |
| DT-000812 | 56.3 | 2.9 | 56.8 | 6.0 |
| DT-001250 | 52.8 | 5.4 | 61.1 | 5.3 |
| DT-001251 | 56.3 | 4.2 | 57.0 | 2.7 |
| DT-001252 | 53.6 | 1.6 | 49.6 | 4.0 |
| DT-001253 | 52.7 | 3.6 | 54.9 | 3.4 |

OLFML2A expression

| | | | | |
|---|---|---|---|---|
| PBS | 105.6 | 12.6 | 104.9 | 12.2 |
| DT-000812 | 84.1 | 11.1 | 81.9 | 7.6 |
| DT-001250 | 74.3 | 10.4 | 66.4 | 5.5 |
| DT-001251 | 86.9 | 6.8 | 65.4 | 8.5 |
| DT-001252 | 72.2 | 8.3 | 54.9 | 8.6 |
| DT-001253 | 71.6 | 6.1 | 72.6 | 8.4 |

DT-000812, DT-001254, DT-001255, DT-001257 28-day efficacy study

The efficacies of DT-001254, DT-001255, and DT-001257 were evaluated in C3-PMP22 mice. DT-000812 was included in the study. Groups of eight mice each were treated with PBS and a single dose of 30 mg/kg of each compound on Day 0 of the study. Wild-type mice treated with PBS were used as a control (WT-PBS). Motor nerve conduction velocity (MNCV) and compound muscle action potential (CMAP) were determined just prior to treatment (Baseline; Day -1) and at Day 27. At Day 28, mice were sacrificed, and sciatic and brachial plexus nerve tissues were harvested for RNA extraction. Human PMP22 mRNA expression was measured by quantitative RT-PCR. The expression of several myelin-specific mouse mRNAs was also measured by quantitative RT-PCR.

The average percent expression for human PMP22 mRNA was calculated for each treatment and is shown in Table 71. MNCV and CMAP are shown in Table 72. The average percent expression for myelin-specific mouse mRNAs was calculated and is shown in Table 73.

In each table, WT-PBS indicates wild-type mice treated with PBS; all other data were obtained in C3-PMP22 mice.

TABLE 71
Human PMP22 mRNA 28 days following a single
dose of 30 mg/kg conjugated PMP22 siRNAs

| | |
|---|---|
| Sciatic | Brachial Plexus |

| Treatment | Mean | S.E.M. | Mean | S.E.M. |
|---|---|---|---|---|
| PBS | 101.2 | 6.0 | 102.5 | 9.6 |
| DT-000812 | 66.8 | 7.6 | 41.8 | 8.0 |
| DT-001254 | 54.2 | 10.1 | 30.3 | 4.6 |
| DT-001255 | 61.8 | 8.7 | 30.1 | 2.4 |
| DT-001257 | 57.9 | 10.2 | 38.8 | 9.3 |

TABLE 72
MNCV and CMAP at Baseline and 27 days following
a single dose of 30 mg/kg conjugated PMP22 siRNAs

| | |
|---|---|
| Baseline | Day 27 |

| | | | | |
|---|---|---|---|---|
| Treatment | Mean | S.E.M. | Mean | S.E.M. |

MNCV

| | | | | |
|---|---|---|---|---|
| WT-PBS | 35.8 | 5.0 | 55.5 | 8.6 |
| PBS | 31.8 | 3.7 | 25.6 | 2.3 |
| DT-000812 | 30.8 | 3.4 | 46.6 | 6.6 |
| DT-001254 | 25.4 | 3.3 | 46.1 | 6.2 |
| DT-001255 | 31.9 | 3.2 | 43.8 | 4.6 |
| DT-001257 | 22.7 | 3.0 | 36.1 | 4.0 |

CMAP

| | | | | |
|---|---|---|---|---|
| WT-PBS | 3.9 | 1.1 | 3.8 | 0.9 |
| PBS | 0.9 | 0.2 | 1.3 | 0.2 |
| DT-000812 | 1.3 | 0.2 | 3.1 | 0.5 |
| DT-001254 | 0.8 | 0.1 | 3.0 | 0.5 |
| DT-001255 | 1.2 | 0.2 | 3.8 | 0.9 |
| DT-001257 | 0.9 | 0.2 | 2.7 | 0.5 |

TABLE 73
Myelin-specific mRNA expression 28 days following
a single dose of 30 mg/kg conjugated PMP22 siRNAs

| | |
|---|---|
| Sciatic | Brachial |

| | | | | |
|---|---|---|---|---|
| Treatment | Mean | S.E.M. | Mean | S.E.M. |

MPZ expression

| | | | | |
|---|---|---|---|---|
| WT-PBS | 122.4 | 5.0 | 153.1 | 17.1 |
| PBS | 101.4 | 6.9 | 102.2 | 7.4 |
| DT-000812 | 184.6 | 13.7 | 163.6 | 4.9 |
| DT-001254 | 167.2 | 15.9 | 157.2 | 9.0 |
| DT-001255 | 190.0 | 18.8 | 162.5 | 8.4 |
| DT-001257 | 172.1 | 10.8 | 146.5 | 18.7 |

Pou3F1 expression

| | | | | |
|---|---|---|---|---|
| PBS | 135.1 | 40.7 | 103.3 | 10.0 |
| DT-000812 | 58.7 | 19.3 | 40.0 | 7.4 |
| DT-001254 | 54.6 | 13.6 | 28.9 | 4.8 |
| DT-001255 | 55.2 | 13.2 | 41.3 | 6.6 |
| DT-001257 | 46.7 | 11.8 | 43.7 | 7.4 |

CXCL14 expression

| | | | | |
|---|---|---|---|---|
| PBS | 105.4 | 12.3 | 102.1 | 7.7 |
| DT-000812 | 53.6 | 4.8 | 20.1 | 3.7 |
| DT-001254 | 67.2 | 9.0 | 24.4 | 3.8 |
| DT-001255 | 72.0 | 10.2 | 18.3 | 2.6 |
| DT-001257 | 64.2 | 7.1 | 28.1 | 7.5 |

NGFR expression

| | | | | |
|---|---|---|---|---|
| PBS | 108.5 | 15.8 | 107.7 | 14.3 |
| DT-000812 | 69.3 | 20.2 | 69.3 | 7.8 |
| DT-001254 | 70.2 | 13.8 | 61.2 | 10.7 |
| DT-001255 | 78.1 | 17.0 | 93.5 | 9.7 |
| DT-001257 | 73.2 | 17.2 | 79.5 | 23.2 |

CSRP2 expression

| | | | | |
|---|---|---|---|---|
| PBS | 106.4 | 12.8 | 113.4 | 22.0 |
| DT-000812 | 49.3 | 6.1 | 36.0 | 5.7 |
| DT-001254 | 43.2 | 5.5 | 38.4 | 10.7 |
| DT-001255 | 59.0 | 8.4 | 31.3 | 5.4 |
| DT-001257 | 52.0 | 5.7 | 39.1 | 7.9 |

CUEDC2 expression

| | | | | |
|---|---|---|---|---|
| PBS | 103.3 | 10.1 | 101.0 | 5.3 |
| DT-000812 | 72.8 | 9.1 | 62.2 | 5.5 |
| DT-001254 | 77.5 | 13.8 | 49.2 | 3.7 |
| DT-001255 | 85.8 | 11.8 | 62.8 | 5.1 |
| DT-001257 | 83.5 | 9.9 | 68.7 | 6.2 |

OLFML2A expression

| | | | | |
|---|---|---|---|---|
| PBS | 138.6 | 33.7 | 110.9 | 15.4 |
| DT-000812 | 108.0 | 23.9 | 56.7 | 12.9 |
| DT-001254 | 87.1 | 19.7 | 68.7 | 6.2 |
| DT-001255 | 87.3 | 17.8 | 72.6 | 12.1 |
| DT-001257 | 131.9 | 33.1 | 93.7 | 13.6 |

### DT-000812, DT-001254, DT-001255, DT-001257 60-Day Efficacy Study

DT-000812, DT-001254, DT-001255, and DT-001257 were evaluated in a 60-day efficacy study in C3-PMP22 mice. Groups of eight mice each were treated with PBS and a single dose of 30 mg/kg of each compound on Day 0 of the study. Wild-type mice treated with PBS were used as a control (WT-PBS). Motor nerve conduction velocity (MNCV) and compound muscle action potential (CMAP) were determined just prior to treatment (Baseline; Day -1), at Day 28 and at Day 59. At Day 60, mice were sacrificed, and sciatic and brachial plexus nerve tissues were harvested for RNA extraction. Human PMP22 mRNA expression was measured by quantitative RT-PCR. The expression of several myelin-specific mouse mRNAs was also measured by quantitative RT-PCR.

The average percent expression for human PMP22 mRNA was calculated for each treatment and is shown in Table 74. MNCV and CMAP are shown in Table 75. The average percent expression for the myelin-specific mRNAs was calculated and is shown in Table 76.

In each table, WT-PBS indicates wild-type mice treated with PBS; all other data were obtained in C3-PMP22 mice.

TABLE 74
Human PMP22 mRNA 60 days following a single
dose of 30 mg/kg conjugated PMP22 siRNAs

| | |
|---|---|
| Sciatic | Brachial Plexus |

| Treatment | Mean | S.E.M. | Mean | S.E.M. |
|---|---|---|---|---|
| WT-PBS | | | | |
| PBS | 100.7 | 4.7 | 101.7 | 7.8 |
| DT-000812 | 70.1 | 7.3 | 78.6 | 6.6 |
| DT-001254 | 37.5 | 4.7 | 34.3 | 3.7 |
| DT-001255 | 54.7 | 5.3 | 39.2 | 5.7 |
| DT-001257 | 49.6 | 6.5 | 34.5 | 5.1 |

TABLE 75
MNCV and CMAP at Baseline and Days 28 and 59 following
a single dose of 30 mg/kg conjugated PMP22 siRNAs

| | Baseline | | Day 28 | | Day 59 | |
|---|---|---|---|---|---|---|
| Treatment | Mean | S.E.M. | Mean | S.E.M. | Mean | S.E.M. |
| | MNCV | | | | | |
| WT-PBS | 52.0 | 6.5 | 73.2 | 10.2 | 48.5 | 5.8 |
| PBS | 27.9 | 2.8 | 49.2 | 5.7 | 30.1 | 3.2 |
| DT-000812 | 29.5 | 4.0 | 69.2 | 7.3 | 39.5 | 3.5 |
| DT-001254 | 23.6 | 2.2 | 62.7 | 8.2 | 43.9 | 4.6 |
| DT-001255 | 26.6 | 4.0 | 52.8 | 5.3 | 33.1 | 4.2 |
| DT-001257 | 24.7 | 1.6 | 51.3 | 3.6 | 48.9 | 6.3 |
| | CMAP | | | | | |
| WT-PBS | 4.5 | 0.8 | 4.7 | 0.9 | 5.7 | 1.2 |
| PBS | 1.3 | 0.2 | 0.9 | 0.2 | 1.5 | 0.2 |
| DT-000812 | 1.2 | 0.2 | 2.5 | 0.5 | 3.3 | 0.6 |
| DT-001254 | 1.2 | 0.2 | 2.6 | 0.9 | 3.7 | 0.9 |
| DT-001255 | 0.9 | 0.2 | 3.3 | 0.9 | 2.6 | 0.5 |
| DT-001257 | 1.3 | 0.3 | 2.8 | 0.4 | 3.5 | 0.7 |

TABLE 76
Myelin-specific mRNA expression 60 days following
a single dose of 30 mg/kg conjugated PMP22 siRNAs

| | |
|---|---|
| Sciatic | Brachial |

| | | | | |
|---|---|---|---|---|
| Treatment | Mean | S.E.M. | Mean | S.E.M. |

MPZ expression

| | | | | |
|---|---|---|---|---|
| WT-PBS | 126.1 | 4.8 | 132.8 | 8.0 |
| PBS | 100.8 | 5.0 | 100.9 | 5.7 |
| DT-000812 | 127.7 | 6.2 | 147.8 | 8.3 |
| DT-001254 | 137.3 | 9.2 | 143.3 | 13.4 |
| DT-001255 | 121.7 | 8.8 | 127.7 | 9.0 |
| DT-001257 | 104.6 | 5.0 | 132.4 | 4.3 |

Pou3F1 expression

| | | | | |
|---|---|---|---|---|
| WT-PBS | 20.6 | 4.8 | 21.7 | 3.8 |
| PBS | 104.0 | 11.2 | 101.6 | 7.3 |
| DT-000812 | 39.7 | 5.0 | 40.9 | 4.5 |
| DT-001254 | 38.3 | 4.7 | 34.7 | 1.4 |
| DT-001255 | 38.9 | 4.8 | 35.9 | 4.6 |
| DT-001257 | 27.2 | 2.4 | 30.1 | 7.3 |

CXCL14 expression

| | | | | |
|---|---|---|---|---|
| WT-PBS | 43.0 | 9.2 | 9.1 | 2.0 |
| PBS | 103.0 | 9.9 | 109.0 | 18.9 |
| DT-000812 | 80.9 | 8.3 | 22.0 | 3.7 |
| DT-001254 | 93.6 | 12.0 | 25.8 | 4.4 |
| DT-001255 | 85.5 | 8.4 | 19.5 | 3.4 |
| DT-001257 | 77.2 | 10.0 | 20.2 | 7.3 |

NGFR expression

| | | | | |
|---|---|---|---|---|
| WT-PBS | 51.8 | 6.4 | 30.9 | 7.0 |
| PBS | 100.9 | 5.3 | 105.0 | 13.2 |
| DT-000812 | 70.6 | 4.4 | 79.9 | 8.1 |
| DT-001254 | 71.4 | 6.0 | 68.2 | 9.4 |
| DT-001255 | 65.4 | 7.1 | 61.1 | 7.3 |
| DT-001257 | 86.5 | 7.6 | 81.0 | 11.2 |

CSRP2 expression

| | | | | |
|---|---|---|---|---|
| WT-PBS | 36.1 | 6.6 | 28.6 | 4.6 |
| PBS | 103.7 | 11.4 | 103.4 | 11.1 |
| DT-000812 | 54.4 | 6.2 | 35.2 | 4.3 |
| DT-001254 | 42.4 | 2.0 | 29.2 | 4.1 |
| DT-001255 | 49.3 | 4.5 | 35.7 | 5.0 |
| DT-001257 | 48.5 | 6.0 | 34.4 | 3.5 |

CUEDC2 expression

| | | | | |
|---|---|---|---|---|
| WT-PBS | 42.1 | 4.0 | 42.5 | 3.4 |
| PBS | 101.7 | 7.5 | 103.3 | 11.6 |
| DT-000812 | 53.4 | 3.0 | 55.1 | 3.5 |
| DT-001254 | 56.1 | 5.1 | 45.4 | 3.0 |
| DT-001255 | 55.9 | 5.0 | 54.0 | 4.0 |
| DT-001257 | 54.9 | 2.1 | 51.3 | 5.9 |

OLFML2A expression

| | | | | |
|---|---|---|---|---|
| WT-PBS | 34.7 | 5.7 | 21.0 | 4.7 |
| PBS | 100.8 | 5.3 | 109.0 | 17.3 |
| DT-000812 | 59.3 | 5.4 | 69.1 | 9.8 |
| DT-001254 | 48.5 | 5.2 | 58.3 | 10.5 |
| DT-001255 | 45.8 | 5.2 | 59.6 | 6.5 |
| DT-001257 | 49.7 | 3.7 | 49.4 | 7.7 |

### DT-000812, DT-001263 28-Day Efficacy Study

The efficacy of DT-001263 was evaluated and compared to DT-000812, in C3-PMP22 mice. Groups of eight mice each were treated with PBS and a single dose of 30 mg/kg of each compound on Day 0 of the study. Wild-type mice treated with PBS were used as a control (WT-PBS). Motor nerve conduction velocity (MNCV) and compound muscle action potential (CMAP) were determined just prior to treatment (Baseline; Day -1) and at Day 27. At Day 28, mice were sacrificed, and sciatic and brachial plexus nerve tissues were harvested for RNA extraction. Human PMP22 mRNA expression was measured by quantitative RT-PCR.

The average percent expression for human PMP22 mRNA was calculated for each treatment and is shown in Table 77. MNCV and CMAP are shown in Table 78. The expression of mouse MPZ mRNA was also measured by quantitative RT-PCR. The average percent expression for each of these mRNAs was calculated and is shown in Table 79.

In each table, WT-PBS indicates wild-type mice treated with PBS; all other data were obtained in C3-PMP22 mice.

TABLE 77
Human PMP22 mRNA 28 days following a single
dose of 30 mg/kg conjugated PMP22 siRNAs

| | |
|---|---|
| Sciatic | Brachial Plexus |

| Treatment | Mean | S.E.M. | Mean | S.E.M. |
|---|---|---|---|---|
| PBS | 100.1 | 1.9 | 100.6 | 4.1 |
| DT-000812 | 81.1 | 4.6 | 58.9 | 5.1 |
| DT-001263 | 65.9 | 4.6 | 47.2 | 4.7 |

TABLE 78
MNCV and CMAP at Baseline and 27 days following
a single dose of 30 mg/kg conjugated PMP22 siRNAs

| | |
|---|---|
| Baseline | Day 27 |

| | | | | |
|---|---|---|---|---|
| Treatment | Mean | S.E.M. | Mean | S.E.M. |

MNCV

| | | | | |
|---|---|---|---|---|
| WT-PBS | 45.8 | 4.6 | 50.9 | 5.4 |
| PBS | 23.8 | 3.1 | 22.1 | 1.0 |
| DT-000812 | 23.1 | 3.5 | 26.6 | 2.8 |
| DT-001263 | 26.7 | 1.9 | 32.0 | 1.8 |

CMAP

| | | | | |
|---|---|---|---|---|
| WT-PBS | 4.0 | 0.5 | 5.3 | 0.5 |
| PBS | 0.9 | 0.1 | 1.6 | 0.3 |
| DT-000812 | 1.0 | 0.2 | 2.2 | 0.4 |
| DT-001263 | 1.0 | 0.2 | 3.5 | 0.9 |

TABLE 79
Myelin-specific mRNA expression 28 days following
a single dose of 30 mg/kg conjugated PMP22 siRNAs
MPZ expression

| | |
|---|---|
| Sciatic | Brachial |

| Treatment | Mean | S.E.M. | Mean | S.E.M. |
|---|---|---|---|---|
| WT-PBS | 173.4 | 6.5 | 151.6 | 6.9 |
| PBS | 100.4 | 3.3 | 100.6 | 4.2 |
| DT-000812 | 172.3 | 7.4 | 143.0 | 16.6 |
| DT-001263 | 181.6 | 14.8 | 186.1 | 8.7 |

12-week Efficacy Studies: DT-001252, DT-001253, and DT-001257

DT-001252, DT-001253, and DT-001257 were each evaluated in separate 12-week efficacy studies in C3-PMP22 mice. Each study also included treatment with DT-000812 at 30 mg/kg. Groups of eight mice each were treated with PBS, or monthly doses of 3 mg/kg, 10 mg/kg, or 30 mg/kg siRNA compound on Day 0, Day 28, and Day 56, for a total of 3 doses. Wild-type mice treated with PBS were used as a control (WT-PBS). Motor nerve conduction velocity (MNCV), compound muscle action potential (CMAP), grip strength and beam walking ability were determined just prior to treatment to establish a baseline value and at 4, 8, and 12 weeks of treatment. At 12 weeks, mice were sacrificed, and sciatic and brachial plexus nerves were harvested for RNA extraction. Human PMP22 mRNA expression in C3-PMP22 mice was measured by quantitative RT-PCR. The expression of several myelin-specific mouse mRNAs was also measured by quantitative RT-PCR. Peripheral nerves were dissected and prepared for morphometric analysis according to routine methods (for example, Jolivalt, et al., 2016, Curr. Protoc. Mouse Biol., 6:223-255). Cross sections of nerve were processed into resin blocks which were cut into 0.5- to 1.3-μm thick sections, stained with p-phenylenediamine, and viewed by light microscopy. Axon diameters and myelin thickness were measured using a software-assisted manual approach in ImageJ/FIJI.

The average percent expression for human PMP22 mRNA was calculated for each treatment and is shown in Table 80. The average percent expression for myelin-specific mRNAs was calculated and is shown in Table 85.

The average MNCV per treatment group at each time point is shown in Table 81. In the experiment testing DT-001252, errors in measurement of the traces resulted in variable MNCV data at the baseline, 4-week and 8-week timepoints, thus these data are not presented. The average CMAP per treatment group at each time point is shown in Table 82. Grip strength and beam walking ability were measured at 4, 8, and 12 weeks and are shown in Table 82.

The mean percentage of unmyelinated axons in each treatment group is shown in Table 83.

In each table, WT-PBS indicates wild-type mice treated with PBS; all other data were obtained in C3-PMP22 mice.

TABLE 80
Human PMP22 mRNA 12 weeks following treatment

| | |
|---|---|
| Sciatic | Brachial Plexus |

| Treatment | Mean | S.E.M. | Mean | S.E.M. |
|---|---|---|---|---|
| PBS | 94.0 | 3.6 | 101.4 | 6.2 |
| 30 mg/kg DT-000812 | 28.0 | 2.8 | 21.3 | 2.4 |
| 3 mg/kg DT-001252 | 69.2 | 6.1 | 50.5 | 2.3 |
| 10 mg/kg DT-001252 | 29.0 | 1.7 | 19.0 | 2.3 |
| 30 mg/kg DT-001252 | 12.3 | 1.6 | 8.2 | 0.4 |
| PBS | 108.3 | 15.5 | 100.7 | 4.5 |
| 30 mg/kg DT-000812 | 15.8 | 1.8 | 14.1 | 0.7 |
| 3 mg/kg DT-001253 | 45.5 | 4.8 | 53.6 | 4.5 |
| 10 mg/kg DT-001253 | 49.5 | 4.0 | 22.9 | 2.0 |
| 30 mg/kg DT-001253 | 14.9 | 1.2 | 12.8 | 0.5 |
| PBS | 101.5 | 6.7 | 102.4 | 8.5 |
| 30 mg/kg DT-000812 | 21.6 | 1.5 | 15.8 | 1.2 |
| 3 mg/kg DT-001257 | 101.4 | 9.1 | 87.7 | 3.7 |
| 10 mg/kg DT-001257 | 67.8 | 6.2 | 37.0 | 4.9 |
| 30 mg/kg DT-001257 | 19.5 | 1.9 | 13.6 | 1.0 |

TABLE 81
MNCV during and following treatment

| | Baseline | | 4 weeks | | 8 weeks | | 12 weeks | |
|---|---|---|---|---|---|---|---|---|
| | Mean | | Mean | | Mean | | Mean | |
| Treatment | m/s | S.E.M. | m/s | S.E.M. | m/s | S.E.M. | m/s | S.E.M. |
| WT- PBS | — | — | — | — | — | — | 43.4 | 5.3 |
| PBS | — | — | — | — | — | — | 18.7 | 1.4 |
| 30 mg/kg DT-000812 | — | — | — | — | — | — | 43.4 | 4.9 |
| 3 mg/kg DT-001252 | — | — | — | — | — | — | 38.0 | 2.3 |
| 10 mg/kg DT-001252 | — | — | — | — | — | — | 37.5 | 2.0 |
| 30 mg/kg DT-001252 | — | — | — | — | — | — | 40.6 | 1.2 |
| WT- PBS | 42.8 | 3.7 | 62.7 | 9.0 | 49.0 | 4.9 | 57.0 | 4.8 |
| PBS | 19.1 | 1.8 | 25.7 | 2.2 | 29.2 | 3.3 | 22.4 | 2.7 |
| 30 mg/kg DT-000812 | 25.0 | 3.9 | 42.0 | 4.7 | 33.7 | 6.2 | 39.0 | 2.4 |
| 3 mg/kg DT-001253 | 26.6 | 2.8 | 31.5 | 0.8 | 30.0 | 1.8 | 38.7 | 4.0 |
| 10 mg/kg DT-001253 | 23.6 | 2.3 | 35.0 | 5.6 | 34.8 | 1.7 | 46.6 | 5.0 |
| 30 mg/kg DT-001253 | 20.6 | 1.9 | 38.5 | 5.2 | 27.1 | 2.3 | 49.2 | 4.1 |
| WT- PBS | 45.5 | 3.0 | 67.6 | 8.6 | 48.9 | 3.8 | 49.4 | 4.5 |
| PBS | 15.8 | 0.9 | 30.0 | 2.8 | 25.1 | 3.5 | 27.9 | 2.0 |
| 30 mg/kg DT-000812 | 21.9 | 1.9 | 44.3 | 6.4 | 33.1 | 1.8 | 43.4 | 2.6 |
| 3 mg/kg DT-001257 | 19.6 | 1.9 | 29.5 | 4.5 | 33.1 | 3.0 | 33.6 | 2.4 |
| 10 mg/kg DT-001257 | 21.8 | 1.3 | 36.6 | 5.4 | 39.1 | 3.9 | 45.5 | 1.5 |
| 30 mg/kg DT-001257 | 20.8 | 1.7 | 43.5 | 5.7 | 31.5 | 2.9 | 38.2 | 0.6 |

TABLE 82
CMAP during and following treatment

| | Baseline | | 4 weeks | | 8 weeks | | 12 weeks | |
|---|---|---|---|---|---|---|---|---|
| | Mean | | Mean | | Mean | | Mean | |
| Treatment | mV | S.E.M. | mV | S.E.M. | mV | S.E.M. | mV | S.E.M. |
| WT- PBS | 4.7 | 1.1 | 4.3 | 1.3 | 3.4 | 0.7 | 3.8 | 0.6 |
| PBS | 1.0 | 0.1 | 0.9 | 0.1 | 1.1 | 0.4 | 1.1 | 0.2 |
| 30 mg/kg DT-000812 | 1.2 | 0.1 | 2.0 | 0.4 | 2.0 | 0.4 | 2.7 | 0.4 |
| 3 mg/kg DT-001252 | 1.3 | 0.2 | 2.1 | 0.5 | 2.6 | 0.4 | 3.8 | 1.2 |
| 10 mg/kg DT-001252 | 1.3 | 0.2 | 2.9 | 0.5 | 3.4 | 1.0 | 4.2 | 0.8 |
| 30 mg/kg DT-001252 | 1.3 | 0.2 | 2.7 | 0.6 | 4.2 | 0.9 | 5.7 | 1.2 |
| WT- PBS | 4.1 | 0.8 | 3.7 | 0.5 | 5.6 | 0.9 | 7.5 | 1.3 |
| PBS | 0.9 | 0.2 | 1.3 | 0.2 | 1.3 | 0.1 | 1.3 | 0.3 |
| 30 mg/kg DT-000812 | 1.2 | 0.2 | 3.2 | 0.5 | 3.0 | 0.9 | 5.0 | 1.0 |
| 3 mg/kg DT-001253 | 1.4 | 0.3 | 1.3 | 0.2 | 3.1 | 0.7 | 3.2 | 0.4 |
| 10 mg/kg DT-001253 | 0.9 | 0.2 | 2.8 | 0.6 | 3.3 | 0.5 | 3.9 | 1.1 |
| 30 mg/kg DT-001253 | 1.0 | 0.2 | 3.6 | 0.8 | 3.2 | 0.8 | 5.0 | 1.0 |
| WT- PBS | 4.1 | 1.0 | 3.4 | 0.6 | 4.1 | 1.0 | 4.0 | 0.8 |
| PBS | 1.5 | 0.2 | 1.5 | 0.4 | 1.8 | 0.6 | 0.9 | 0.2 |
| 30 mg/kg DT-000812 | 1.1 | 0.2 | 3.2 | 0.5 | 2.3 | 0.6 | 3.2 | 0.5 |
| 3 mg/kg DT-001257 | 1.1 | 0.2 | 1.7 | 0.2 | 1.6 | 0.3 | 1.5 | 0.3 |
| 10 mg/kg DT-001257 | 1.7 | 0.2 | 2.5 | 0.6 | 1.8 | 0.4 | 2.3 | 0.4 |
| 30 mg/kg DT-001257 | 1.4 | 0.2 | 4.2 | 1.1 | 3.0 | 0.7 | 2.5 | 0.4 |

TABLE 83
Grip strength during and following treatment

| | Baseline | | 4 weeks | | 8 weeks | | 12 weeks | |
|---|---|---|---|---|---|---|---|---|
| | Mean | | Mean | | Mean | | Mean | |
| Treatment | g | S.E.M. | g | S.E.M. | g | S.E.M. | g | S.E.M. |
| WT- PBS | 156.1 | 5.8 | 162.0 | 6.2 | 144.2 | 8.9 | 166.7 | 7.9 |
| PBS | 102.4 | 3.6 | 104.2 | 4.3 | 84.8 | 7.0 | 106.0 | 5.6 |
| 30 mg/kg DT-000812 | 102.6 | 5.5 | 148.7 | 6.3 | 139.9 | 7.5 | 170.2 | 8.6 |
| 3 mg/kg DT-001252 | 118.4 | 6.4 | 122.1 | 4.3 | 118.9 | 5.1 | 144.9 | 7.5 |
| 10 mg/kg DT-001252 | 104.0 | 3.2 | 127.4 | 5.7 | 120.5 | 5.2 | 155.2 | 4.9 |
| 30 mg/kg DT-001252 | 106.6 | 9.2 | 133.3 | 5.6 | 135.9 | 5.9 | 165.5 | 9.0 |
| WT- PBS | 147.4 | 5.0 | 152.1 | 5.1 | 162.2 | 10.7 | 169.8 | 6.4 |
| PBS | 112.9 | 7.3 | 88.2 | 6.9 | 79.3 | 6.3 | 86.8 | 7.3 |
| 30 mg/kg DT-000812 | 112.2 | 3.3 | 130.8 | 6.8 | 139.5 | 8.8 | 154.3 | 6.9 |
| 3 mg/kg DT-001253 | 106.6 | 2.8 | 109.5 | 5.7 | 125.8 | 6.9 | 144.9 | 4.1 |
| 10 mg/kg DT-001253 | 114.2 | 4.3 | 132.3 | 8.0 | 135.1 | 8.3 | 155.2 | 3.9 |
| 30 mg/kg DT-001253 | 122.6 | 4.7 | 141.8 | 7.7 | 144.0 | 9.6 | 158.6 | 6.7 |
| WT- PBS | 151.7 | 7.3 | 165.0 | 6.2 | 154.9 | 8.5 | 184.0 | 6.0 |
| PBS | 125.2 | 2.2 | 102.8 | 11.6 | 100.4 | 10.9 | 115.6 | 13.5 |
| 30 mg/kg DT-000812 | 109.7 | 6.1 | 124.1 | 3.9 | 140.7 | 5.2 | 175.5 | 6.3 |
| 3 mg/kg DT-001257 | 118.9 | 3.2 | 103.6 | 5.2 | 120.3 | 5.2 | 130.0 | 2.4 |
| 10 mg/kg DT-001257 | 113.8 | 3.5 | 123.4 | 4.6 | 140.6 | 5.3 | 164.1 | 6.2 |
| 30 mg/kg DT-001257 | 120.1 | 3.3 | 158.5 | 7.5 | 146.1 | 9.5 | 163.4 | 5.1 |

TABLE 84
Slips while crossing beam during and following treatment

| | Baseline | | 4 weeks | | 8 weeks | | 12 weeks | |
|---|---|---|---|---|---|---|---|---|
| | Mean | | Mean | | Mean | | Mean | |
| Treatment | # slips | SEM | # slips | SEM | # slips | SEM | # slips | SEM |
| WT- PBS | 3.7 | 0.7 | 3.2 | 0.7 | 5.1 | 1.0 | 6.0 | 1.7 |
| PBS | 20.1 | 2.8 | 14.4 | 1.9 | 32.4 | 2.2 | 19.2 | 3.2 |
| 30 mg/kg DT-000812 | 18.6 | 3.8 | 9.5 | 2.2 | 9.8 | 1.8 | 7.3 | 2.3 |
| 3 mg/kg DT-001252 | 17.1 | 4.3 | 8.8 | 2.5 | 11.7 | 2.7 | 10.7 | 1.8 |
| 10 mg/kg DT-001252 | 18.6 | 4.4 | 5.6 | 1.0 | 12.2 | 2.4 | 6.3 | 2.6 |
| 30 mg/kg DT-001252 | 16.9 | 2.9 | 7.3 | 0.8 | 8.5 | 1.6 | 7.6 | 1.3 |
| WT- PBS | 9.8 | 1.9 | 5.5 | 0.9 | 5.9 | 1.5 | 5.0 | 0.8 |
| PBS | 30.7 | 4.7 | 24.7 | 5.1 | 32.0 | 3.5 | 36.0 | 5.0 |
| 30 mg/kg DT-000812 | 30.7 | 2.9 | 7.9 | 1.1 | 6.1 | 1.2 | 5.8 | 1.0 |
| 3 mg/kg DT-001253 | 26.4 | 4.3 | 11.4 | 2.8 | 13.6 | 2.1 | 15.6 | 2.6 |
| 10 mg/kg DT-001253 | 36.6 | 3.5 | 7.7 | 1.2 | 5.8 | 0.7 | 7.2 | 1.5 |
| 30 mg/kg DT-001253 | 31.2 | 3.3 | 6.5 | 1.4 | 4.8 | 1.1 | 7.0 | 1.3 |
| WT- PBS | 7.1 | 1.6 | 9.7 | 2.7 | 5.9 | 1.7 | 6.8 | 1.7 |
| PBS | 22.5 | 3.8 | 11.0 | 2.1 | 22.6 | 4.1 | 20.9 | 2.8 |
| 30 mg/kg DT-000812 | 22.4 | 3.3 | 11.5 | 3.0 | 12.4 | 1.7 | 9.8 | 3.3 |
| 3 mg/kg DT-001257 | 26.6 | 5.8 | 17.0 | 3.7 | 11.8 | 1.8 | 13.7 | 2.4 |
| 10 mg/kg DT-001257 | 26.2 | 3.9 | 10.5 | 2.7 | 10.4 | 2.4 | 8.0 | 2.4 |
| 30 mg/kg DT-001257 | 24.0 | 2.5 | 8.8 | 2.0 | 8.7 | 2.2 | 7.0 | 1.6 |

TABLE 85
Time to cross beam during and following
treatment with DT-001252

| | Baseline | | 4 weeks | | 8 weeks | | 12 weeks | |
|---|---|---|---|---|---|---|---|---|
| | Mean | | Mean | | Mean | | Mean | |
| Treatment | sec. | SEM | sec. | SEM | sec. | SEM | sec. | SEM |
| WT- PBS | 14.8 | 1.6 | 12.1 | 2.1 | 15.3 | 1.2 | 14.8 | 1.8 |
| PBS | 27.6 | 2.9 | 21.2 | 2.9 | 29.5 | 1.9 | 28.4 | 3.3 |
| 30 mg/kg DT- | 26.5 | 4.6 | 21.9 | 5.5 | 21.9 | 3.0 | 19.7 | 4.7 |
| 000812 | | | | | | | | |
| 3 mg/kg DT- | 26.0 | 3.9 | 19.4 | 2.7 | 20.7 | 2.0 | 23.2 | 1.2 |
| 001252 | | | | | | | | |
| 10 mg/kg DT- | 27.5 | 5.6 | 13.3 | 1.8 | 17.1 | 2.9 | 16.0 | 3.3 |
| 001252 | | | | | | | | |
| 30 mg/kg DT- | 24.0 | 4.4 | 15.2 | 1.5 | 16.0 | 2.6 | 19.0 | 2.4 |
| 001252 | | | | | | | | |
| WT- PBS | 17.0 | 1.5 | 15.2 | 1.6 | 14.8 | 1.5 | 21.4 | 2.0 |
| PBS | 24.3 | 2.8 | 19.7 | 2.5 | 28.5 | 2.1 | 30.0 | 3.1 |
| 30 mg/kg DT- | 28.9 | 3.3 | 15.1 | 2.4 | 19.2 | 2.7 | 19.9 | 3.0 |
| 000812 | | | | | | | | |
| 3 mg/kg DT- | 25.0 | 2.2 | 17.7 | 2.8 | 25.0 | 4.2 | 24.1 | 3.0 |
| 001253 | | | | | | | | |
| 10 mg/kg DT- | 30.6 | 1.7 | 12.3 | 1.0 | 14.7 | 1.0 | 21.8 | 2.6 |
| 001253 | | | | | | | | |
| 30 mg/kg DT- | 28.8 | 2.3 | 14.0 | 1.1 | 17.4 | 1.3 | 26.5 | 2.6 |
| 001253 | | | | | | | | |
| WT- PBS | 14.7 | 2.8 | 15.2 | 3.7 | 10.8 | 1.4 | 15.4 | 2.3 |
| PBS | 27.3 | 3.0 | 20.5 | 2.2 | 26.2 | 3.1 | 22.0 | 2.3 |
| 30 mg/kg DT- | 28.9 | 3.8 | 22.0 | 2.4 | 22.9 | 5.5 | 17.7 | 2.5 |
| 000812 | | | | | | | | |
| 3 mg/kg DT- | 28.6 | 4.0 | 22.3 | 2.5 | 20.8 | 2.4 | 20.2 | 2.0 |
| 001257 | | | | | | | | |
| 10 mg/kg DT- | 29.0 | 4.9 | 20.8 | 2.6 | 17.9 | 2.8 | 15.2 | 2.3 |
| 001257 | | | | | | | | |
| 30 mg/kg DT- | 26.8 | 2.3 | 20.0 | 4.2 | 22.3 | 2.2 | 16.3 | 2.2 |
| 001257 | | | | | | | | |

TABLE 86
Myelin-specific mRNA expression following weekly
injections of 10 mg/kg or monthly injections
of 30 mg/kg of conjugated siRNA compound

| | |
|---|---|
| Sciatic | Brachial Plexus |

| | | | | |
|---|---|---|---|---|
| Treatment | Mean | S.E.M. | Mean | S.E.M. |

MPZ expression

| | | | | |
|---|---|---|---|---|
| WT- PBS | 89.1 | 3.8 | 112.9 | 5.1 |
| PBS | 95.3 | 5.6 | 101.2 | 6.1 |
| 30 mg/kg DT-000812 | 114.2 | 7.3 | 151.4 | 7.7 |
| 3 mg/kg DT-001252 | 122.7 | 6.5 | 137.5 | 8.9 |
| 10 mg/kg DT-001252 | 132.3 | 6.3 | 158.0 | 12.0 |
| 30 mg/kg DT-001252 | 136.3 | 8.7 | 133.8 | 10.9 |
| WT- PBS | 169.3 | 16.0 | 136.9 | 13.8 |
| PBS | 111.6 | 20.1 | 100.3 | 2.8 |
| 30 mg/kg DT-000812 | 185.0 | 19.5 | 145.0 | 4.4 |
| 3 mg/kg DT-001253 | 184.8 | 27.0 | 138.4 | 13.4 |
| 10 mg/kg DT-001253 | 160.1 | 26.8 | 175.6 | 9.7 |
| 30 mg/kg DT-001253 | 216.0 | 13.5 | 175.7 | 10.2 |
| WT- PBS | 103.7 | 16.8 | 120.8 | 7.8 |
| PBS | 102.1 | 7.9 | 100.9 | 5.0 |
| 30 mg/kg DT-000812 | 176.9 | 21.1 | 142.9 | 7.7 |
| 3 mg/kg DT-001257 | 131.7 | 16.4 | 129.9 | 8.4 |
| 10 mg/kg DT-001257 | 212.8 | 27.1 | 141.4 | 7.9 |
| 30 mg/kg DT-001257 | 174.2 | 39.1 | 143.7 | 11.4 |

Pou3F1 expression

| | | | | |
|---|---|---|---|---|
| WT- PBS | 9.8 | 0.6 | 13.0 | 2.3 |
| PBS | 107.0 | 17.8 | 107.0 | 14.3 |
| 30 mg/kg DT-000812 | 20.4 | 2.0 | 30.3 | 3.7 |
| 3 mg/kg DT-001252 | 34.6 | 6.1 | 41.7 | 4.5 |
| 10 mg/kg DT-001252 | 20.6 | 2.7 | 32.6 | 3.6 |
| 30 mg/kg DT-001252 | 16.8 | 4.3 | 24.9 | 3.4 |
| WT- PBS | 23.2 | 6.3 | 28.7 | 5.8 |
| PBS | 107.9 | 16.9 | 103.3 | 9.9 |
| 30 mg/kg DT-000812 | 30.3 | 2.5 | 30.5 | 3.9 |
| 3 mg/kg DT-001253 | 33.9 | 4.4 | 43.7 | 3.5 |
| 10 mg/kg DT-001253 | 37.9 | 5.1 | 38.6 | 5.6 |
| 30 mg/kg DT-001253 | 28.0 | 3.7 | 32.8 | 5.1 |
| WT- PBS | 29.3 | 4.3 | 27.4 | 3.7 |
| PBS | 110.3 | 16.9 | 109.1 | 16.3 |
| 30 mg/kg DT-000812 | 125.2 | 12.0 | 91.6 | 14.1 |
| 3 mg/kg DT-001257 | 76.1 | 7.8 | 59.7 | 6.0 |
| 10 mg/kg DT-001257 | 54.9 | 3.9 | 46.1 | 4.6 |
| 30 mg/kg DT-001257 | 58.1 | 4.7 | 43.5 | 4.3 |

CXCL14 expression

| | | | | |
|---|---|---|---|---|
| WT- PBS | 37.1 | 10.5 | 11.1 | 4.6 |
| PBS | 103.6 | 8.9 | 108.0 | 15.4 |
| 30 mg/kg DT-000812 | 51.6 | 5.9 | 9.3 | 1.9 |
| 3 mg/kg DT-001252 | 54.0 | 5.6 | 21.6 | 2.3 |
| 10 mg/kg DT-001252 | 45.1 | 5.8 | 14.5 | 2.5 |
| 30 mg/kg DT-001252 | 39.2 | 3.6 | 8.0 | 1.6 |
| WT- PBS | 54.3 | 14.1 | 11.3 | 3.3 |
| PBS | 111.0 | 20.8 | 105.1 | 11.6 |
| 30 mg/kg DT-000812 | 37.5 | 8.5 | 22.6 | 2.8 |
| 3 mg/kg DT-001253 | 55.0 | 11.7 | 26.7 | 4.6 |
| 10 mg/kg DT-001253 | 58.9 | 12.8 | 21.2 | 2.2 |
| 30 mg/kg DT-001253 | 33.6 | 4.4 | 22.6 | 1.9 |
| WT- PBS | 62.1 | 12.5 | 5.4 | 1.0 |
| PBS | 115.3 | 20.0 | 140.2 | 31.7 |
| 30 mg/kg DT-000812 | 38.8 | 5.7 | 18.4 | 1.9 |
| 3 mg/kg DT-001257 | 102.2 | 23.5 | 76.3 | 7.3 |
| 10 mg/kg DT-001257 | 38.4 | 3.1 | 26.4 | 5.5 |
| 30 mg/kg DT-001257 | 54.8 | 10.7 | 17.9 | 3.8 |

NGFR expression

| | | | | |
|---|---|---|---|---|
| WT- PBS | 18.8 | 2.3 | 15.0 | 1.4 |
| PBS | 106.6 | 12.9 | 103.8 | 9.6 |
| 30 mg/kg DT-000812 | 50.9 | 5.1 | 42.7 | 4.5 |
| 3 mg/kg DT-001252 | 67.3 | 7.9 | 65.2 | 14.1 |
| 10 mg/kg DT-001252 | 39.5 | 6.1 | 36.3 | 5.4 |
| 30 mg/kg DT-001252 | 41.1 | 7.3 | 35.5 | 3.0 |
| WT- PBS | 47.9 | 6.5 | 24.7 | 2.8 |
| PBS | 101.4 | 6.8 | 100.7 | 4.4 |
| 30 mg/kg DT-000812 | 53.6 | 5.1 | 45.7 | 4.9 |
| 3 mg/kg DT-001253 | 57.2 | 6.7 | 60.2 | 5.2 |
| 10 mg/kg DT-001253 | 68.6 | 5.5 | 49.1 | 5.6 |
| 30 mg/kg DT-001253 | 57.0 | 7.4 | 60.0 | 5.1 |
| WT- PBS | 45.0 | 12.7 | 29.8 | 4.8 |
| PBS | 112.4 | 20.4 | 105.7 | 12.2 |
| 30 mg/kg DT-000812 | 52.1 | 8.3 | 62.1 | 3.4 |
| 3 mg/kg DT-001257 | 101.3 | 17.9 | 92.0 | 5.7 |
| 10 mg/kg DT-001257 | 53.2 | 5.2 | 68.1 | 7.2 |
| 30 mg/kg DT-001257 | 69.9 | 16.3 | 55.9 | 5.8 |

Sox4 expression

| | | | | |
|---|---|---|---|---|
| WT- PBS | 28.3 | 3.0 | 26.5 | 4.6 |
| PBS | 103.4 | 9.2 | 108.0 | 17.9 |
| 30 mg/kg DT-000812 | 38.9 | 5.4 | 39.1 | 6.4 |
| 3 mg/kg DT-001252 | 48.7 | 6.4 | 43.2 | 9.7 |
| 10 mg/kg DT-001252 | 44.8 | 5.0 | 47.2 | 12.9 |
| 30 mg/kg DT-001252 | 27.4 | 3.6 | 28.3 | 5.6 |
| WT- PBS | 48.4 | 6.2 | 23.4 | 8.4 |
| PBS | 101.2 | 6.5 | 104.1 | 10.8 |
| 30 mg/kg DT-000812 | 53.7 | 3.9 | 35.9 | 4.1 |
| 3 mg/kg DT-001253 | 62.9 | 4.4 | 47.8 | 7.8 |
| 10 mg/kg DT-001253 | 68.1 | 4.3 | 51.7 | 10.0 |
| 30 mg/kg DT-001253 | 56.2 | 5.1 | 54.5 | 12.7 |
| WT- PBS | 53.0 | 8.2 | 26.5 | 5.7 |
| PBS | 107.7 | 15.7 | 108.4 | 15.4 |
| 30 mg/kg DT-000812 | 63.4 | 8.1 | 43.9 | 5.4 |
| 3 mg/kg DT-001257 | 103.5 | 12.9 | 79.4 | 6.4 |
| 10 mg/kg DT-001257 | 64.8 | 6.2 | 47.1 | 9.6 |
| 30 mg/kg DT-001257 | 65.4 | 8.8 | 45.9 | 5.6 |

CSRP2 expression

| | | | | |
|---|---|---|---|---|
| WT- PBS | 20.5 | 2.2 | 23.8 | 4.2 |
| PBS | 102.5 | 8.1 | 108.2 | 14.8 |
| 30 mg/kg DT-000812 | 28.5 | 2.2 | 30.7 | 3.7 |
| 3 mg/kg DT-001252 | 39.4 | 4.0 | 36.1 | 5.7 |
| 10 mg/kg DT-001252 | 26.1 | 3.1 | 41.7 | 12.2 |
| 30 mg/kg DT-001252 | 29.0 | 3.3 | 42.9 | 11.5 |
| WT- PBS | 41.2 | 3.9 | 19.7 | 3.3 |
| PBS | 105.8 | 13.2 | 107.2 | 14.7 |
| 30 mg/kg DT-000812 | 38.2 | 2.4 | 28.9 | 5.1 |
| 3 mg/kg DT-001253 | 45.4 | 1.9 | 35.2 | 3.9 |
| 10 mg/kg DT-001253 | 45.0 | 2.4 | 31.5 | 3.4 |
| 30 mg/kg DT-001253 | 46.0 | 3.6 | 34.9 | 4.4 |
| WT- PBS | 41.0 | 4.7 | 21.4 | 3.9 |
| PBS | 114.3 | 22.8 | 119.0 | 22.2 |
| 30 mg/kg DT-000812 | 51.0 | 3.5 | 50.2 | 8.2 |
| 3 mg/kg DT-001257 | 94.6 | 5.2 | 87.6 | 8.3 |
| 10 mg/kg DT-001257 | 54.4 | 5.7 | 35.0 | 6.0 |
| 30 mg/kg DT-001257 | 48.9 | 5.8 | 35.5 | 5.9 |

CUEDC2 expression

| | | | | |
|---|---|---|---|---|
| WT- PBS | 29.3 | 1.5 | 42.6 | 5.2 |
| PBS | 103.9 | 11.0 | 104.1 | 10.4 |
| 30 mg/kg DT-000812 | 39.2 | 2.7 | 56.0 | 9.8 |
| 3 mg/kg DT-001252 | 56.6 | 3.7 | 61.9 | 7.1 |
| 10 mg/kg DT-001252 | 46.0 | 3.4 | 53.3 | 5.3 |
| 30 mg/kg DT-001252 | 46.7 | 5.6 | 52.4 | 7.0 |
| WT- PBS | 45.1 | 3.1 | 44.1 | 4.3 |
| PBS | 105.6 | 12.5 | 101.3 | 6.1 |
| 30 mg/kg DT-000812 | 58.7 | 4.3 | 55.2 | 3.2 |
| 3 mg/kg DT-001253 | 63.2 | 6.5 | 63.2 | 3.6 |
| 10 mg/kg DT-001253 | 62.8 | 6.0 | 59.8 | 3.9 |
| 30 mg/kg DT-001253 | 66.1 | 4.0 | 55.8 | 3.8 |
| WT- PBS | 49.8 | 4.3 | 40.9 | 3.1 |
| PBS | 106.2 | 13.9 | 105.9 | 12.3 |
| 30 mg/kg DT-000812 | 85.6 | 5.0 | 58.7 | 5.9 |
| 3 mg/kg DT-001257 | 108.3 | 8.8 | 108.4 | 13.7 |
| 10 mg/kg DT-001257 | 94.7 | 9.0 | 60.8 | 2.4 |
| 30 mg/kg DT-001257 | 80.1 | 7.2 | 64.9 | 5.2 |

OLFML2A expression

| | | | | |
|---|---|---|---|---|
| WT- PBS | 37.4 | 4.2 | 22.3 | 2.2 |
| PBS | 104.0 | 10.6 | 101.0 | 5.4 |
| 30 mg/kg DT-000812 | 57.1 | 4.2 | 51.4 | 5.2 |
| 3 mg/kg DT-001252 | 66.2 | 7.3 | 70.0 | 8.8 |
| 10 mg/kg DT-001252 | 49.8 | 5.6 | 59.4 | 5.8 |
| 30 mg/kg DT-001252 | 47.6 | 6.4 | 47.4 | 6.2 |
| WT- PBS | 56.9 | 2.9 | 45.4 | 10.1 |
| PBS | 106.0 | 12.6 | 117.8 | 19.2 |
| 30 mg/kg DT-000812 | 74.8 | 5.9 | 78.4 | 8.9 |
| 3 mg/kg DT-001253 | 84.2 | 10.1 | 100.9 | 11.4 |
| 10 mg/kg DT-001253 | 89.4 | 11.7 | 92.7 | 8.5 |
| 30 mg/kg DT-001253 | 82.5 | 8.5 | 100.0 | 18.1 |
| WT- PBS | 52.9 | 4.3 | 25.2 | 3.9 |
| PBS | 104.3 | 11.7 | 103.8 | 11.0 |
| 30 mg/kg DT-000812 | 79.2 | 3.8 | 68.0 | 8.0 |
| 3 mg/kg DT-001257 | 105.6 | 11.2 | 84.2 | 7.4 |
| 10 mg/kg DT-001257 | 93.5 | 9.0 | 61.6 | 7.4 |
| 30 mg/kg DT-001257 | 86.2 | 7.7 | 54.9 | 8.8 |

As illustrated by the above data, substantial improvements in multiple endpoints associated with CMT1A were observed.

Treatment of C3-PMP22 mice with each conjugated PMP22 siRNA tested resulted in a reduction in human PMP22 mRNA expression compared to PBS-treated C3-PMP22 mice in both the sciatic and brachial plexus nerves (Table 80).

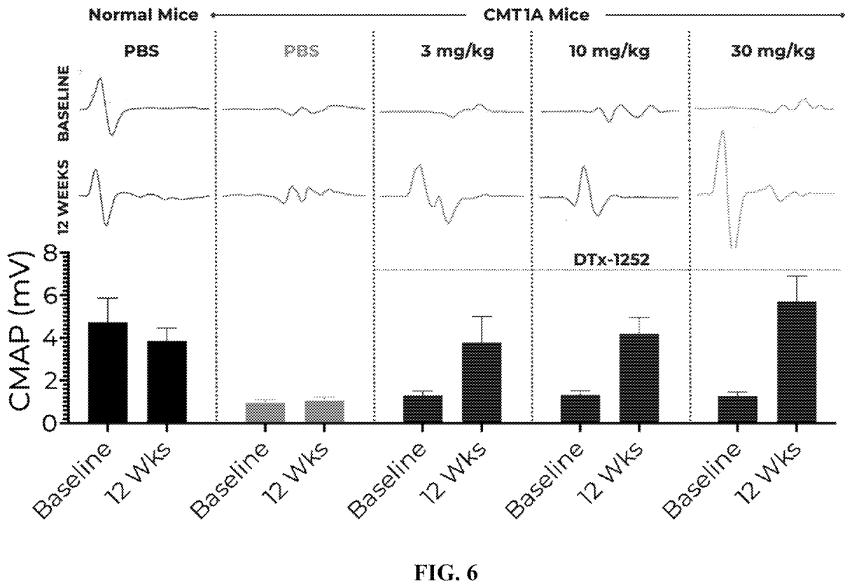

The MNCV tests revealed an improvement in the efficiency of motor nerve conduction at 12 weeks (Table 81). Additionally, each conjugated PMP22 siRNA tested improved compound muscle action potential at each time point (Table 82). The improvement in CMAP following treatment with DT-001252 is further illustrated in FIG. 6. In wild-type mice, CMAP consisted of a strong electrical polarization signal, followed by a depolarization signal. The amplitude, or the differential voltage between the baseline (zero) and the peak of the electrical polarization signal, is readily apparent. In C3-PMP22 mice, the polarization and depolarization signals were muted and difficult to distinguish from background electrical impulses. In contrast, treatment with DT-001252 restored the amplitude of CMAPs in C3-PMP22 mice.

The grip strength of C3-PMP22 mice mice treated with PBS was markedly reduced relative to wild-type mice. Treatment with the conjugated PMP22 siRNAs increased grip strength (Table 83). Furthermore, increases in the masses of several peripheral muscles (quadricep, tibialis anterior and gastrocnemius) were increased relative to untreated C3-PMP22 mice. In the beam walking test, wild-type mice easily traversed the entire length of the beam. In contrast, PBS-treated C3-PMP22 mice proceeded much more slowly, and their hind paws repeatedly slipped off the beam and on average required additional time to travel the same distance as wild-type mice. After treatment with the conjugated PMP22 siRNAs, the speed at which C3-PMP22 mice traversed the beam was closer to that of wild-type mice (Table 85). Additionally, the number of slips relative to PBS-treated C3-PMP22 mice was reduced (Table 84).

Measurement of myelin-specific genes essential for Schwann cell function illustrated that treatment with the conjugated PMP22 siRNAs restored gene expression of these genes in the sciatic and brachial plexus nerves to the levels observed in wild-type mice (Table 83).

Taken, these data demonstrate that inhibition of PMP22 with conjugated PMP22 siRNAs, in an experimental model for CMT1A, leads to substantial improvements in multiple phenotypes associated with CMT1A.

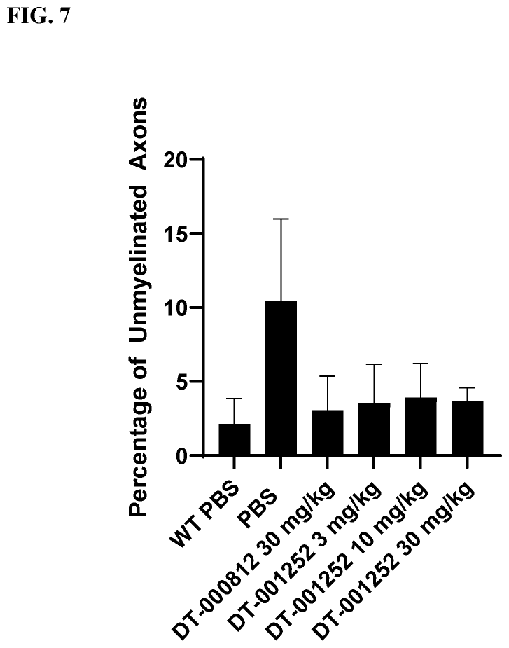

The efficacy of DT-001252 was further evaluated by measuring myelination of the femoral motor nerve. Peripheral nerves were dissected and prepared for morphometric analysis according to routine methods (for example, Jolivalt, et al., 2016, Curr. Protoc. Mouse Biol., 6:223-255). Cross sections of nerve were processed into resin blocks which were cut into 0.5- to 1.3-μm thick sections, stained with p-phenylenediamine, and viewed by light microscopy. Axon diameters and myelin thickness were measured using a software-assisted manual approach in ImageJ/FIJI. Histological analysis revealed that, whereas unmyelinated axons were common in femoral motor nerve sections from C3-PMP22 mice, each DT-001252 treatment group exhibited substantially lower numbers of large unmyelinated axons (Table 87, FIG. 7). Thus, the improvement in MNCV shown in Table 78 is likely due to an increase in the number of myelinated axons in C3-PMP22 mice. The increase in myelinated neurons following treatment with DT-001252 is consistent with the improvements in muscle function observed in grip strength and beam walking tests.

TABLE 87
Quantiation of myelination of peripheral nerves at 12 weeks

| | Percentage of | | |
|---|---|---|---|
| | Unmyleinated Axons | | |
| Treatment | Mean | S.E.M. | # animals |
| WT-PBS | 2.1% | 0.7% | 6 |
| PBS | 10.5% | 2.5% | 5 |
| 30 mg/kg DT-001252 | 3.1% | 0.9% | 6 |
| 3 mg/kg DT-001252 | 3.6% | 1.1% | 6 |
| 10 mg/kg DT-001252 | 3.9% | 1.1% | 4 |
| 30 mg/kg DT-000812 | 3.7% | 0.5% | 4 |

The effect of treatment with DT-001252 on serum Neurofilament light (NfL) was also evaluated. NfL is a marker of neuronal damage and is elevated in subjects with CMT1A. Serum NfL at 12 weeks was measured using a NFL-light Advantage assay kit (Quanterix). The mean NfL for each treatment group is shown in Table 88 (n=7 for PBS-treated C3-PMP22 mice due to exclusion of one outlier individual data point; n=8 for all other groups). As shown in Table 88, treatment with each dose of DT-001252 normalized serum NfL.

TABLE 88
Quantitation of serum NfL

Serum NfL
pg/ml

| Treatment | Mean | S.E.M. |
|---|---|---|
| WT-PBS | 163.5 | 21.8 |
| PBS | 359.1 | 48.9 |
| 3 mg/kg DT-001252 | 272.6 | 61.5 |
| 10 mg/kg DT-001252 | 248.6 | 17.2 |
| 30 mg/kg DT-001252 | 206.5 | 22.9 |

### Additional Compounds: 14-Day Efficacy Study

Additional compounds were designed to evaluate the effects of chemical modifications on the potency of certain conjugated PMP22 siRNAs related to unconjugated compounds identified as “hits” and shown in Table 19. These derivatives comprise the identical nucleotide sequences as their respective parent compounds but have variations in nucleotide modifications. DT-001842 and DT-001843 are derivatives of DT-000901; DT-001844 and DT-001845 are derivatives of DT-000847; DT-001846 and DT-001847 are derivatives of DT-000849; DT-001848 and DT-001849 are derivatives of DT-000855; DT-001858, DT-001859, and DT-001860 are derivatives of DT-000414. Groups of five C3-PMP22 mice each were treated with a single dose of PBS, or 10 mg/kg or 30 mg/kg of conjugated siRNA compound. DT-001252 was included in each study as a benchmark compound. At Day 14 following injection, mice were sacrificed, and sciatic and brachial plexus nerve tissues were harvested for RNA extraction. Human PMP22 mRNA and mouse MPZ mRNA were measured by quantitative RT-PCR. The average percent expression for each mRNA was calculated for each treatment and is shown in Tables 89 through 94. As illustrated in the tables below, derivatives of DT-001252 exhibited potency comparable to that of DT-001252.

TABLE 89
Human PMP22 mRNA 14 days following a single injection
of 10 mg/kg or 30 mg/kg of conjugated siRNA compound
Sciatic Nerve

| | PBS | | 10 mg/kg | | 30 mg/kg | |
|---|---|---|---|---|---|---|
| Treatment | Mean | S.E.M. | Mean | S.E.M. | Mean | S.E.M. |
| PBS | 100.4 | 4.9 | — | — | — | — |
| DT-001252 | — | — | 62.4 | 4.5 | 29.3 | 3.5 |
| DT-001842 | — | — | 105.4 | 3.9 | 89.6 | 3.9 |
| DT-001843 | — | — | 89.4 | 3.6 | 70.0 | 4.1 |
| DT-001844 | — | — | 90.5 | 6.0 | 88.2 | 4.8 |
| DT-001845 | — | — | 102.6 | 6.8 | 103.1 | 3.9 |

Brachial Plexus

| | PBS | | 10 mg/kg | | 30 mg/kg | |
|---|---|---|---|---|---|---|
| | Mean | S.E.M. | Mean | S.E.M. | Mean | S.E.M. |
| PBS | 100.1 | 2.3 | — | — | — | — |
| DT-001252 | — | — | 31.9 | 4.3 | 21.3 | 2.0 |
| DT-001842 | — | — | 98.4 | 1.6 | 81.1 | 4.1 |
| DT-001843 | — | — | 81.5 | 3.8 | 55.5 | 7.2 |
| DT-001844 | — | — | 102.0 | 8.3 | 93.3 | 4.2 |
| DT-001845 | — | — | 92.6 | 1.7 | 103.5 | 8.5 |

TABLE 90
Mouse MPZ mRNA 14 days following a single injection
of 10 mg/kg or 30 mg/kg of conjugated siRNA compound
Sciatic Nerve

| | PBS | | 10 mg/kg | | 30 mg/kg | |
|---|---|---|---|---|---|---|
| Treatment | Mean | S.E.M. | Mean | S.E.M. | Mean | S.E.M. |
| PBS | 100.3 | 3.9 | — | — | — | — |
| DT-001252 | — | — | 174.3 | 11.4 | 187.5 | 17.0 |
| DT-001842 | — | — | 114.4 | 10.2 | 144.1 | 3.4 |
| DT-001843 | — | — | 104.7 | 2.2 | 135.1 | 11.2 |
| DT-001844 | — | — | 86.6 | 4.0 | 85.1 | 4.4 |
| DT-001845 | — | — | 106.8 | 17.9 | 98.3 | 4.8 |

Brachial Plexus

| | PBS | | 10 mg/kg | | 30 mg/kg | |
|---|---|---|---|---|---|---|
| | Mean | S.E.M. | Mean | S.E.M. | Mean | S.E.M. |
| PBS | 100.6 | 5.6 | — | — | — | — |
| DT-001252 | — | — | 192.9 | 8.9 | 226.3 | 13.0 |
| DT-001842 | — | — | 139.1 | 9.9 | 159.3 | 2.1 |
| DT-001843 | — | — | 137.6 | 7.2 | 169.8 | 13.1 |
| DT-001844 | — | — | 106.7 | 9.3 | 114.1 | 8.9 |
| DT-001845 | — | — | 117.5 | 12.4 | 111.1 | 6.4 |

TABLE 91
Human PMP22 mRNA 14 days following a single injection
of 10 mg/kg or 30 mg/kg of conjugated siRNA compound
Sciatic Nerve

| | PBS | | 10 mg/kg | | 30 mg/kg | |
|---|---|---|---|---|---|---|
| Treatment | Mean | S.E.M. | Mean | S.E.M. | Mean | S.E.M. |
| PBS | 100.5 | 5.2 | — | — | — | — |
| DT-001252 | — | — | 57.9 | 6.6 | 28.9 | 2.3 |
| DT-001846 | — | — | 89.7 | 1.7 | 72.3 | 3.6 |
| DT-001847 | — | — | 101.5 | 5.4 | 61.8 | 8.2 |
| DT-001848 | — | — | 117.4 | 3.5 | 99.0 | 5.4 |
| DT-001849 | — | — | 108.0 | 8.0 | 101.1 | 2.8 |

Brachial Plexus

| | PBS | | 10 mg/kg | | 30 mg/kg | |
|---|---|---|---|---|---|---|
| | Mean | S.E.M. | Mean | S.E.M. | Mean | S.E.M. |
| PBS | 100.2 | 2.8 | — | — | — | — |
| DT-001252 | — | — | 35.0 | 6.0 | 21.1 | 1.9 |
| DT-001846 | — | — | 79.9 | 4.0 | 42.3 | 6.1 |
| DT-001847 | — | — | 71.4 | 7.5 | 31.5 | 10.1 |
| DT-001848 | — | — | 104.3 | 5.5 | 90.9 | 5.5 |
| DT-001849 | — | — | 87.7 | 5.1 | 83.9 | 2.2 |

TABLE 92
Mouse MPZ mRNA 14 days following a single injection
of 10 mg/kg or 30 mg/kg of conjugated siRNA compound
Sciatic Nerve

| | PBS | | 10 mg/kg | | 30 mg/kg | |
|---|---|---|---|---|---|---|
| Treatment | Mean | S.E.M. | Mean | S.E.M. | Mean | S.E.M. |
| PBS | 101.5 | 8.8 | — | — | — | — |
| DT-001252 | — | — | 159.8 | 7.0 | 216.3 | 20.8 |
| DT-001846 | — | — | 122.4 | 6.9 | 183.8 | 17.7 |
| DT-001847 | — | — | 152.5 | 10.6 | 181.3 | 8.4 |
| DT-001848 | — | — | 114.1 | 3.1 | 120.6 | 5.2 |
| DT-001849 | — | — | 120.3 | 5.1 | 130.5 | 7.6 |

Brachial Plexus

| | PBS | | 10 mg/kg | | 30 mg/kg | |
|---|---|---|---|---|---|---|
| | Mean | S.E.M. | Mean | S.E.M. | Mean | S.E.M. |
| PBS | 101.8 | 9.6 | — | — | — | — |
| DT-001252 | — | — | 134.3 | 26.4 | 202.2 | 8.4 |
| DT-001846 | — | — | 123.6 | 5.9 | 171.3 | 3.3 |
| DT-001847 | — | — | 157.4 | 10.9 | 175.4 | 9.7 |
| DT-001848 | — | — | 116.9 | 5.1 | 130.1 | 9.2 |
| DT-001849 | — | — | 109.6 | 12.5 | 138.0 | 9.2 |

TABLE 93
Human PMP22 mRNA 14 days following a single injection
of 10 mg/kg or 30 mg/kg of conjugated siRNA compound
Sciatic Nerve

| | PBS | | 10 mg/kg | | 30 mg/kg | |
|---|---|---|---|---|---|---|
| Treatment | Mean | S.E.M. | Mean | S.E.M. | Mean | S.E.M. |
| PBS | 100.6 | 5.3 | — | — | — | — |
| DT-001252 | — | — | 68.4 | 0.7 | 42.4 | 6.3 |
| DT-001858 | — | — | 76.2 | 2.2 | 64.2 | 8.7 |
| DT-001859 | — | — | 66.0 | 3.4 | 30.2 | 1.5 |
| DT-001860 | — | — | 89.6 | 10.2 | 57.3 | 8.2 |

Brachial Plexus

| | PBS | | 10 mg/kg | | 30 mg/kg | |
|---|---|---|---|---|---|---|
| | Mean | S.E.M. | Mean | S.E.M. | Mean | S.E.M. |
| PBS | 100.5 | 5.0 | — | — | — | — |
| DT-001252 | — | — | 54.3 | 5.0 | 37.5 | 4.9 |
| DT-001858 | — | — | 63.7 | 3.7 | 47.2 | 10.7 |
| DT-001859 | — | — | 50.2 | 4.1 | 25.0 | 4.6 |
| DT-001860 | — | — | 77.3 | 7.6 | 55.6 | 8.4 |

TABLE 94
Mouse MPZ mRNA 14 days following a single injection
of 10 mg/kg or 30 mg/kg of conjugated siRNA compound
Sciatic Nerve

| | PBS | | 10 mg/kg | | 30 mg/kg | |
|---|---|---|---|---|---|---|
| Treatment | Mean | S.E.M. | Mean | S.E.M. | Mean | S.E.M. |
| PBS | 101.1 | 9.0 | — | — | — | — |
| DT-001252 | — | — | 165.3 | 4.6 | 182.2 | 6.3 |
| DT-001858 | — | — | 126.6 | 9.4 | 176.0 | 11.7 |
| DT-001859 | — | — | 143.0 | 10.7 | 174.0 | 2.3 |
| DT-001860 | — | — | 99.3 | 16.0 | 136.1 | 5.9 |

Brachial Plexus

| | PBS | | 10 mg/kg | | 30 mg/kg | |
|---|---|---|---|---|---|---|
| | Mean | S.E.M. | Mean | S.E.M. | Mean | S.E.M. |
| PBS | 100.8 | 6.6 | — | — | — | — |
| DT-001252 | — | — | 184.1 | 12.1 | 187.3 | 26.6 |
| DT-001858 | — | — | 141.8 | 11.0 | 159.9 | 8.5 |
| DT-001859 | — | — | 139.4 | 6.7 | 141.8 | 12.6 |
| DT-001860 | — | — | 120.0 | 10.4 | 154.8 | 15.4 |

Comparison of Activity of Structurally Related Conjugated PMP22 siRNAs

As illustrated herein, certain conjugated PMP22 siRNAs exhibited potent reduction of hPMP22 in the C3-PMP22 mouse model. One such group of related siRNAs is listed in Table 95. Each of these siRNAs has the sense strand of SEQ ID NO: 1015 or SEQ ID NO: 1018 (which differ by a single nucleobase), the antisense strand of SEQ ID NO: 1144 and the DTx-01-08 motif conjugated to the 3′ end of the sense strand through a C7 linker as described herein. As each antisense strand of each siRNA has the nucleotide sequence of SEQ ID NO: 1144, each siRNA targets nucleotides 213 to 233 of the human PMP22 mRNA. Variations were introduced in the number, nature, and placement of chemical modifications, as shown in Table 95. Each % hPMP22 shown in Table 95 is from an experiment described herein and is reproduced below for comparison. While each of the conjugated PMP22 siRNAs in Table 95 exhibits potent reduction of the hPMP22 mRNA, certain analogs including but not limited to DT-001252 and DT-001253 are notable for their duration of action.

TABLE 95
Potency of structurally related conjugated PMP22 siRNAs

| | % hPMP22 remaining | | | | Strand | |
|---|---|---|---|---|---|---|
| | 14 days | 14 days | 30 days | 60 days | ID | |
| siRNA | 10 | 30 | 30 | 30 | (SEQ ID | Sequence and Chemistry |
| ID | mg/kg | mg/kg | mg/kg | mg/kg | NO) | (5′ to 3′) |
| DT- | 79.9 | 54.1 | 63.7 | 46.1 | DTS- | 5′-OH- |
| 000812 | | | | | 001217 | C_{F}^{S}C_{M}^{S}U_{F}C_{M}C_{F}U_{M}G_{F}U_{M}U_{F}G_{M}C_{F}U_{M}G_{F} |
| | | | | | (547) | A_{M}G_{F}U_{M}A_{F}U_{M}C_{F}^{S}A_{M}^{S}U_{F}-C7OH-[DTx- |
| | | | | | | 01-08] |
| | | | | | DTS- | 5′-VP- |
| | | | | | 001218 | A_{M}^{S}U_{F}^{S}G_{M}A_{F}U_{M}A_{F}C_{M}U_{F}C_{M}A_{F}G_{M}C_{F}A_{M} |
| | | | | | (912) | A_{F}C_{M}A_{F}G_{M}G_{F}A_{M}G_{F}G_{M}^{S}A_{M}^{S}G_{M}-OH-3′ |
| DT- | 64.5 | 33.9 | 50.8 | 58.3 | DTS- | 5′-OH- |
| 001246 | | | | | 001887 | C_{F}^{S}C_{M}^{S}U_{F}C_{M}C_{F}U_{M}G_{F}U_{M}U_{F}G_{M}C_{F}U_{F}G_{F}A |
| | | | | | (774) | _{M}G_{F}U_{M}A_{F}U_{M}C_{F}^{S}A_{M}^{S}U_{F}-C7OH-[DTx- |
| | | | | | | 01-08] |
| | | | | | DTS- | 5′-VP- |
| | | | | | 001888 | A_{M}^{S}U_{F}^{S}G_{M}A_{F}U_{M}A_{F}C_{M}U_{F}C_{M}A_{M}G_{M}C_{F}A |
| | | | | | (1083) | _{M}A_{F}C_{M}A_{F}G_{M}G_{F}A_{M}G_{F}G_{M}^{S}A_{M}^{S}G_{M}-OH-3′ |
| DT- | 57.8 | 37.2 | 62.3 | 55.2 | DTS- | 5′-OH- |
| 001247 | | | | | 001889 | C_{F}^{S}C_{M}^{S}U_{F}C_{M}C_{F}U_{M}G_{F}U_{M}U_{F}G_{F}C_{F}U_{M}G_{F}A |
| | | | | | (775) | _{M}G_{F}U_{M}A_{F}U_{M}C_{F}^{S}A_{M}^{S}U_{F}-C7OH-[DTx- |
| | | | | | | 01-08] |
| | | | | | DTS- | 5′-VP- |
| | | | | | 001890 | A_{M}^{S}U_{F}^{S}G_{M}A_{F}U_{M}A_{F}C_{M}U_{F}C_{M}A_{F}G_{M}C_{M}A |
| | | | | | (1084) | _{M}A_{F}C_{M}A_{F}G_{M}G_{F}A_{M}G_{F}G_{M}^{S}A_{M}^{S}G_{M}-OH-3′ |
| DT- | 105.2 | 40.8 | 38.9 | 59.0 | DTS- | 5′-OH- |
| 001250 | | | | | 001893 | C_{M}^{S}C_{M}^{S}U_{M}C_{M}C_{F}U_{M}G_{F}U_{M}U_{F}G_{M}C_{F}U_{M}G_{F} |
| | | | | | (776) | A_{M}G_{F}U_{M}A_{F}U_{M}C_{M}^{S}A_{M}^{S}U_{M}-C7OH- |
| | | | | | | [DTx-01-08] |
| | | | | | DTS- | 5′-VP- |
| | | | | | 001218 | A_{M}^{S}U_{F}^{S}G_{M}A_{F}U_{M}A_{F}C_{M}U_{F}C_{M}A_{F}G_{M}C_{F}A_{M} |
| | | | | | (912) | A_{F}C_{M}A_{F}G_{M}G_{F}A_{M}G_{F}G_{M}^{S}A_{M}^{S}G_{M}-OH-3′ |
| DT- | 117.2 | 51.5 | 47.0 | 74.2 | DTS- | 5′-OH- |
| 001251 | | | | | 001894 | C_{M}^{S}C_{M}^{S}U_{M}C_{M}C_{M}U_{M}G_{F}U_{M}U_{F}G_{M}C_{F}U_{M}G |
| | | | | | (777) | _{F}A_{M}G_{F}U_{M}A_{F}U_{M}C_{M}^{S}A_{M}^{S}U_{M}-C7OH- |
| | | | | | | [DTx-01-08] |
| | | | | | DTS- | 5′-VP- |
| | | | | | 001895 | A_{M}^{S}U_{F}^{S}G_{M}A_{F}U_{M}A_{F}C_{M}U_{M}C_{M}A_{F}G_{M}C_{M}A |
| | | | | | (1085) | _{M}A_{F}C_{M}A_{F}G_{M}G_{F}A_{M}G_{F}G_{M}^{S}A_{M}^{S}G_{M}-OH-3′ |
| DT- | 79.8 | 61.1 | 25.2 | 33.9 | DTS- | 5′-OH- |
| 001252 | | | | | 001896 | C_{M}^{S}C_{M}^{S}U_{M}C_{M}C_{M}U_{M}G_{F}U_{M}U_{F}G_{F}C_{F}U_{M}G |
| | | | | | (778) | _{M}A_{M}G_{M}U_{M}A_{M}U_{M}C_{M}^{S}A_{M}^{S}U_{M}-C7OH- |
| | | | | | | [DTx-01-08] |
| | | | | | DTS- | 5′-VP- |
| | | | | | 001897 | A_{M}^{S}U_{F}^{S}G_{M}A_{M}U_{M}A_{F}C_{M}U_{M}C_{M}A_{M}G_{M}C_{M} |
| | | | | | (1086) | A_{M}A_{F}C_{M}A_{F}G_{M}G_{M}A_{M}G_{M}G_{M}^{S}A_{M}^{S}G_{M}- |
| | | | | | | OH-3′ |
| DT- | 88.3 | 53.2 | 26.0 | 35.0 | DTS- | 5′-OH- |
| 001253 | | | | | 001898 | C_{M}^{S}C_{M}^{S}U_{M}C_{M}C_{M}U_{M}G_{F}U_{M}U_{F}G_{F}C_{F}U_{M}G |
| | | | | | (779) | _{M}A_{M}G_{M}U_{M}A_{M}U_{M}C_{M}A_{M}U_{M}-C7OH- |
| | | | | | | [DTx-01-08] |
| | | | | | DTS- | 5′-VP- |
| | | | | | 001897 | A_{M}^{S}U_{F}^{S}G_{M}A_{M}U_{M}A_{F}C_{M}U_{M}C_{M}A_{M}G_{M}C_{M} |
| | | | | | (1086) | A_{M}A_{F}C_{M}A_{F}G_{M}G_{M}A_{M}G_{M}G_{M}^{S}A_{M}^{S}G_{M}- |
| | | | | | | OH-3′ |
| DT- | 82.0 | 40.0 | 54.2 | 37.5 | DTS- | 5′-OH- |
| 001254 | | | | | 001899 | C_{E}^{S}C_{E}^{S}U_{M}C_{M}C_{F}U_{M}G_{F}U_{M}U_{F}G_{M}C_{F}U_{M}G_{F} |
| | | | | | (780) | A_{M}G_{F}U_{M}A_{F}U_{M}C_{M}^{S}A_{M}^{S}U_{M}-C7OH- |
| | | | | | | [DTx-01-08] |
| | | | | | DTS- | 5′-VP- |
| | | | | | 001218 | A_{M}^{S}U_{F}^{S}G_{M}A_{F}U_{M}A_{F}C_{M}U_{F}C_{M}A_{F}G_{M}C_{F}A_{M} |
| | | | | | (912) | A_{F}C_{M}A_{F}G_{M}G_{F}A_{M}G_{F}G_{M}^{S}A_{M}^{S}G_{M}-OH-3′ |
| DT- | 73.4 | 33.9 | 61.8 | 54.7 | DTS- | 5′-OH- |
| 001255 | | | | | 001900 | C_{M}^{S}CE^{S}UEC_{M}C_{F}U_{M}G_{F}U_{M}U_{F}G_{M}C_{F}U_{M}G_{F} |
| | | | | | (781) | A_{M}G_{F}U_{M}A_{F}U_{M}C_{M}^{S}A_{M}^{S}U_{M}-C7OH- |
| | | | | | | [DTx-01-08] |
| | | | | | DTS- | 5′-VP- |
| | | | | | 001218 | A_{M}^{S}U_{F}^{S}G_{M}A_{F}U_{M}A_{F}C_{M}U_{F}C_{M}A_{F}G_{M}C_{F}A_{M} |
| | | | | | (912) | A_{F}C_{M}A_{F}G_{M}G_{F}A_{M}G_{F}G_{M}^{S}A_{M}^{S}G_{M}-OH-3′ |
| DT- | 67.7 | 28.8 | 57.9 | 49.6 | DTS- | 5′-OH- |
| 001257 | | | | | 001902 | C_{E}^{S}C_{E}^{S}UECEC_{F}U_{M}G_{F}U_{M}U_{F}G_{M}C_{F}U_{M}G_{F}A |
| | | | | | (783) | _{M}G_{F}U_{M}A_{F}U_{M}C_{M}^{S}A_{M}^{S}U_{M}-C7OH-[DTx- |
| | | | | | | 01-08] |
| | | | | | DTS- | 5′-VP- |
| | | | | | 001218 | A_{M}^{S}U_{F}^{S}G_{M}A_{F}U_{M}A_{F}C_{M}U_{F}C_{M}A_{F}G_{M}C_{F}A_{M} |
| | | | | | (912) | A_{F}C_{M}A_{F}G_{M}G_{F}A_{M}G_{F}G_{M}^{S}A_{M}^{S}G_{M}-OH-3′ |

| | | | | | |
|---|---|---|---|---|---|
| DT- | 126.6 | 64.2 | — | DTS- | 5′-HO- |
| 001858 | | | | 002898 | C_{M}^{S}C_{M}^{S}U_{M}C_{M}C_{M}U_{M}G_{F}U_{M}U_{F}G_{F}C_{F}U_{M}G |
| | | | | (1156) | _{M}A_{M}G_{M}U_{M}A_{M}U_{M}C_{M}A_{M}^{S}U_{M}-C7OH- |
| | | | | | [DTx-01-08] |
| | | | | DTS- | 5′-VP- |
| | | | | 001897 | A_{M}^{S}U_{F}^{S}G_{M}A_{M}U_{M}A_{F}C_{M}U_{M}C_{M}A_{M}G_{M}C_{M} |
| | | | | (1086) | A_{M}A_{F}C_{M}A_{F}G_{M}G_{M}A_{M}G_{M}G_{M}^{S}A_{M}^{S}G_{M}- |
| | | | | | OH-3′ |
| DT- | 143.0 | 30.2 | — | DTS- | 5′-HO- |
| 001859 | | | | 002899 | C_{M}^{S}C_{M}^{S}U_{F}C_{M}C_{M}U_{M}G_{F}U_{M}U_{F}G_{F}C_{F}U_{M}G_{M} |
| | | | | (1157) | A_{M}G_{M}U_{M}A_{M}U_{M}C_{M}^{S}A_{M}^{S}U_{M}-C7OH- |
| | | | | | [DTx-01-08] |
| | | | | DTS- | 5′-VP- |
| | | | | 001897 | A_{M}^{S}U_{F}^{S}G_{M}A_{M}U_{M}A_{F}C_{M}U_{M}C_{M}A_{M}G_{M}C_{M} |
| | | | | (1086) | A_{M}A_{F}C_{M}A_{F}G_{M}G_{M}A_{M}G_{M}G_{M}^{S}A_{M}^{S}G_{M}- |
| | | | | | OH-3′ |
| DT- | 99.3 | 57.3 | — | DTS- | 5′-HO- |
| 001860 | | | | 001896 | C_{M}^{S}C_{M}^{S}U_{M}C_{M}C_{M}U_{M}G_{F}U_{M}U_{F}G_{F}C_{F}U_{M}G |
| | | | | (778) | _{M}A_{M}G_{M}U_{M}A_{M}U_{M}C_{M}^{S}A_{M}^{S}U_{M}-C7OH- |
| | | | | | DTx-01-08 |
| | | | | DTS- | 5′-VP- |
| | | | | 002900 | A_{M}^{S}U_{F}^{S}G_{M}A_{M}U_{M}A_{F}C_{M}U_{M}C_{M}A_{M}G_{M}C_{M} |
| | | | | (1166) | A_{M}A_{F}C_{M}A_{F}G_{M}G_{M}A_{M}G_{M}G_{M}^{S}A_{M}^{S}GEOH- |
| | | | | | 3′ |

1. A compound, or a pharmaceutical salt thereof, comprising an antisense strand and a sense strand hybridized to form a double-stranded nucleic acid, wherein each of the antisense strand and sense strands is 15 to 25 nucleotides in length, the nucleotide sequence of the antisense strand is at least 90% complementary to the human peripheral myelin protein 22 mRNA (SEQ ID NO: 1170), and the nucleotide sequence of the sense strand has no more than two mismatches to the nucleotide sequence of the antisense strand in the double-stranded region; wherein the compound is a structure of formula (I): 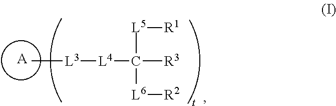
wherein A is the antisense strand and/or the sense strand of the double-stranded nucleic acid;
wherein t is an integer from 1 to 5;
L³ and L⁴ are independently a bond, —N(R²³)—, —O—, —S—, —C(O)—, —N(R²³)C(O)—, —C(O)N(R²⁴)—, —N(R²³)C(O)N(R²⁴)—, —C(O)O—, —OC(O)—, —N(R²³)C(O)O—, —OC(O)N(R²⁴)—, —OPO₂—O—, —O—P(O)(S)—O—, —O—P(O)(R²⁵)—O—, —O—P(S)(R²⁵)—O—, —O—P(O)(NR²³R²⁴)—N—, —O—P(S)(NR²³R²⁴)—N—, —O—P(O)(NR²³R²⁴)—O—, —O—P(S)(NR²³R²⁴)—O—, —P(O)(NR²³R²⁴)—N—, —P(S)(NR²³R²⁴)—N—, —P(O)(NR²³R²⁴)—O—, —P(S)(NR²³R²⁴)—O—, —S—S—, substituted or unsubstituted alkylene, substituted or unsubstituted heteroalkylene, substituted or unsubstituted cycloalkylene, substituted or unsubstituted heterocycloalkylene, substituted or unsubstituted arylene or substituted or unsubstituted heteroarylene;
L⁵ is -L^{5A}-L^{5B}-L^{5C}-L^{5D}-L^{5E}-;
L⁶ is -L^{6A}-L^{6B}-L^{6C}-L^{6D}-L^{6E}-;
R¹ and R² are independently unsubstituted C₁-C₂₅ alkyl, wherein at least one of R¹ and R² is unsubstituted C₉-C₁₉ alkyl;
R³ is hydrogen, —NH₂, —OH, —SH, —C(O)H, —C(O)NH₂, —NHC(O)H, —NHC(O)OH, —NHC(O)NH₂, —C(O)OH, —OC(O)H, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
L^{5A}, L^{5B}, L^{5C}, L^{5D}, L^{5E}, L^{6A}, L^{6B}, L^{6C}, L^{6D}, and L^{6E} are independently a bond, —NH—, —O—, —S—, —C(O)—, —NHC(O)—, —NHC(O)NH—, —C(O)O—, —OC(O)—, —C(O)NH—, substituted or unsubstituted alkylene, substituted or unsubstituted heteroalkylene, substituted or unsubstituted cycloalkylene, substituted or unsubstituted heterocycloalkylene, substituted or unsubstituted arylene or substituted or unsubstituted heteroarylene; and
each R²³, R²⁴ and R²⁵ is independently hydrogen or unsubstituted C₁-C₁₀ alkyl

2-32. (canceled)

33. The compound of claim 1, or a pharmaceutical salt thereof, wherein the antisense strand and the sense strand are not covalently linked.

34. The compound of claim 1, or a pharmaceutical salt thereof,
wherein the hybridization of the antisense strand to the sense strand forms at least one blunt end.

35-37. (canceled)

38. The compound of claim 33, or a pharmaceutical salt thereof, wherein the antisense strand comprises the 3′ nucleotide overhang.

39-42. (canceled)

43. The compound of claim 33, or a pharmaceutical salt thereof, wherein the 3′ nucleotide overhang is two nucleotides in length.

44. (canceled)

45. The compound of claim 1, or a pharmaceutical salt thereof, wherein at least one nucleotide of the antisense strand is a modified nucleotide; and at least one nucleotide of the sense strand is a modified nucleotide.

46-48. (canceled)

49. The compound of claim 1, or a pharmaceutical salt thereof, wherein each nucleotide of the antisense and sense strands is a modified nucleotide.

50. (canceled)

51. The compound of claim 45, or a pharmaceutical salt thereof, wherein the modified nucleotide comprises one or more of a modified sugar moiety, a modified internucleotide linkage, and a 5′-terminal modified phosphate group.

52. The compound of claim 51, or a pharmaceutical salt thereof, wherein the modified nucleotide comprising a modified sugar moiety is selected from a 2′-fluoro nucleotide, a 2′-O-methyl nucleotide, a 2′-O-methoxyethyl nucleotide, and a bicyclic sugar nucleotide.

53. (canceled)

54. The compound of claim 1, or a pharmaceutical salt thereof, wherein the first two internucleotide linkages at the 5′ terminus of the sense strand and the last two internucleotide linkages at the 3′ terminus of the sense strand are phosphorothioate internucleotide linkages; and the first two internucleotide linkages at the 5′ terminus of the antisense strand and the last two internucleotide linkages at the 3′ terminus of the antisense strand are phosphorothioate internucleotide linkages.

55. (canceled)

57. The compound of claim 51, or a pharmaceutical salt thereof, wherein the 5′-terminal modified phosphate group is a 5′-(E)-vinylphosphonate.

58-65. (canceled)

65. The compound of claim 1, or a pharmaceutical salt thereof, wherein the antisense strand is 23 nucleotides in length and the nucleotides of the antisense strand are modified such that, counting from the 5′ terminus of the antisense strand, nucleotides 1, 3, 4, 5, 7, 8, 9, 10, 11, 12, 13, 15, 17, 18, 19, 20, 21, 22, and 23 are 2′-O-methyl nucleotides, nucleotides 2, 6, 14, and 16 are 2′-fluoro nucleotides, the first two internucleotide linkages at the 5′ terminus and the last two internucleotide linkages at the 3′ terminus are phosphorothioate internucleotide linkages, and each other internucleotide linkage is a phosphodiester internucleotide linkage; and wherein the sense strand is 21 nucleotides in length and the nucleotides of the sense strand are modified such that, counting from the 5′ terminus of the sense strand, nucleotides 1, 2, 3, 4, 5, 6, 8, 12, 13, 14, 15, 16, 17, 18, 19, 20, and 21 are 2′-O-methyl nucleotides, nucleotides 7, 9, 10, and 11 are 2′-fluoro nucleotides, the first two internucleotide linkages at the 5′ terminus and the last two internucleotide linkages at the 3′ terminus are phosphorothioate internucleotide linkages, and each other internucleotide linkage is a phosphodiester internucleotide linkage.

66-70. (canceled)

71. The compound of claim 57, or a pharmaceutical salt thereof, wherein the 5′ terminal phosphate group of the antisense strand is a 5′-(E)-vinylphosphonate group.

72. The compound of claim 1, or a pharmaceutical salt thereof, wherein the compound comprises a ligand covalently linked to one or more of the antisense strand and the sense strand of the double-stranded nucleic acid.

73-141. (canceled)

142. The compound of claim 1, or a pharmaceutical salt thereof, wherein -L³-L⁴-
is 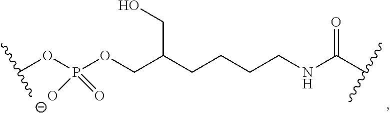 [SMILES: *OP(=O)([O-])OCC(CO)CCCCNC(*)=O]
the phosphate group of -L³-L⁴- is attached to the 3′ carbon of the 3′ terminal nucleotide of the sense strand,
L⁶ is 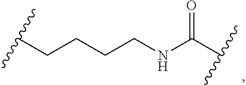 [SMILES: *CCCCNC(*)=O]
L⁵ is —NHC(O)—,
R³ is hydrogen,
R¹ is unsubstituted unbranched C₁₅ alkyl, and
R² is unsubstituted unbranched C₁₅ alkyl.

143. (canceled)

144. The compound of claim 1, or a pharmaceutical salt thereof, wherein the compound is selected from the group consisting of DT-000544 (SEQ ID NOS: 646 and 167), DT-000545 (SEQ ID NOS: 647 and 172), DT-000546 (SEQ ID NOS: 648 and 174), DT-000620 (SEQ ID NOS: 649 and 172), DT-000621 (SEQ ID NOS: 650 and 158), DT-000622 (SEQ ID NOS: 651 and 160), DT-000623 (SEQ ID NOS: 646 and 167), DT-000624 (SEQ ID NOS: 653 and 180), DT-000625 (SEQ ID NOS: 654 and 182), DT-000626 (SEQ ID NOS: 655 and 183), DT-000627 (SEQ ID NOS: 656 and 184), DT-000628 (SEQ ID NOS: 657 and 187), DT-000811 (SEQ ID NOS: 652 and 188), DT-000812 (SEQ ID NOS: 658 and 879), DT-000945 (SEQ ID NOS: 658 and 880), DT-000959 (SEQ ID NOS: 659 and 189), DT-000960 (SEQ ID NOS: 660 and 190), DT-000961 (SEQ ID NOS: 661 and 191), DT-000962 (SEQ ID NOS: 662 and 193), DT-000963 (SEQ ID NOS: 663 and 197), DT-000964 (SEQ ID NOS: 664 and 209), DT-000965 (SEQ ID NOS: 665 and 217), DT-000966 (SEQ ID NOS: 666 and 219), DT-000967 (SEQ ID NOS: 667 and 220), DT-001037 (SEQ ID NOS: 668 and 879), DT-001038 (SEQ ID NOS: 669 and 298), DT-001039 (SEQ ID NOS: 670 and 298), DT-001044 (SEQ ID NOS: 671 and 299), DT-001045 (SEQ ID NOS: 672 and 300), DT-001046 (SEQ ID NOS: 673 and 301), DT-001047 (SEQ ID NOS: 674 and 302), DT-001048 (SEQ ID NOS: 675 and 303), DT-001049 (SEQ ID NOS: 676 and 304), DT-001050 (SEQ ID NOS: 677 and 305), DT-001051 (SEQ ID NOS: 678 and 306), DT-001052 (SEQ ID NOS: 679 and 307), DT-001053 (SEQ ID NOS: 680 and 308), DT-001054 (SEQ ID NOS: 681 and 309), DT-001055 (SEQ ID NOS: 682 and 310), DT-001056 (SEQ ID NOS: 683 and 311), DT-001057 SE ID NOS: 684 and 312), DT-001058 (SEQ ID NOS: 685 and 313), DT-001059 (SEQ ID NOS: 686 and 314), DT-001060 (SEQ ID NOS: 687 and 315), DT-001061 (SEQ ID NOS: 688 and 316), DT-001109 (SEQ ID NOS: 689 and 249), DT-001110 (SEQ ID NOS: 690 and 250), DT-001111 (SEQ ID NOS: 691 and 251), DT-001112 (SEQ ID NOS: 692 and 252), DT-001113 SE ID NOS: 693 and 253), DT-001114 (SEQ ID NOS: 694 and 255), DT-001115 (SEQ ID NOS: 695 and 256), DT-001116 (SEQ ID NOS: 696 and 257), DT-001117 (SEQ ID NOS: 697 and 258), DT-001118 (SEQ ID NOS: 698 and 259), DT-001119 (SEQ ID NOS: 699 and 260), DT-001120 (SEQ ID NOS: 700 and 272), DT-001121 (SEQ ID NOS: 689 and 323), DT-001122 SE ID NOS: 690 and 324), DT-001123 (SEQ ID NOS: 691 and 325), DT-001124 (SEQ ID NOS: 692 and 326), DT-001125 (SEQ ID NOS: 693 and 327), DT-001126 (SEQ ID NOS: 694 and 328), DT-001127 (SEQ ID NOS: 695 and 329), DT-001128 (SEQ ID NOS: 696 and 330), DT-001129 (SEQ ID NOS: 697 and 331), DT-001130 (SEQ ID NOS: 698 and 332), DT-001131 SEQ ID NOS: 699 and 333), DT-001132 (SEQ ID NOS: 700 and 334), DT-001145 (SEQ ID NOS: 701 and 323, DT-001146 (SEQ ID NOS: 702 and 324), DT-001147 (SEQ ID NOS: 703 and 326), DT-001148 (SEQ ID NOS: 704 and 331), DT-001149 (SEQ ID NOS: 705 and 325), DT-001150 (SEQ ID NOS: 706 and 327), DT-001151 (SEQ ID NOS: 707 and 328), DT-001152 SEQ ID NOS: 708 and 329), DT-001153 (SEQ ID NOS: 709 and 330), DT-001154 (SEQ ID NOS: 710 and 332, DT-001155 (SEQ ID NOS: 711 and 333), DT-001156 (SEQ ID NOS: 712 and 334), DT-001157 (SEQ ID NOS: 713 and 317), DT-001158 (SEQ ID NOS: 714 and 318), DT-001159 (SEQ ID NOS: 715 and 319), DT-001160 (SEQ ID NOS: 716 and 320), DT-001161 SE ID NOS: 717 and 267), DT-001162 (SEQ ID NOS: 718 and 170), DT-001163 (SEQ ID NOS: 719 and 321, DT-001164 (SEQ ID NOS: 720 and 322), DT-001176 (SEQ ID NOS: 721 and 192), DT-001177 (SEQ ID NOS: 722 and 194), DT-001178 (SEQ ID NOS: 723 and 196), DT-001179 (SEQ ID NOS: 724 and 199), DT-001180 (SEQ ID NOS: 725 and 200), DT-001181 SEQ ID NOS: 726 and 202), DT-001182 (SEQ ID NOS: 727 and 210), DT-001183 (SEQ ID NOS: 728 and 212), DT-001184 (SEQ ID NOS: 729 and 213), DT-001185 (SEQ ID NOS: 730 and 216), DT-001186 (SEQ ID NOS: 731 and 221), DT-001187 (SEQ ID NOS: 732 and 293), DT-001188 (SEQ ID NOS: 733 and 294), DT-001189 (SEQ ID NOS: 734 and 295), DT-001190 (SEQ ID NOS: 722 and 335), DT-001191 (SEQ ID NOS: 723 and 336), DT-001192 (SEQ ID NOS: 724 and 337), DT-001193 (SEQ ID NOS: 725 and 338), DT-001194 (SEQ ID NOS: 726 and 339), DT-001195 (SEQ ID NOS: 735 and 223), DT-001196 (SEQ ID NOS: 736 and 224), DT-001197 (SEQ ID NOS: 737 and 225), DT-001198 (SEQ ID NOS: 669 and 226), DT-001199 (SEQ ID NOS: 738 and 227), DT-001200 (SEQ ID NOS: 739 and 228), DT-001201 (SEQ ID NOS: 740 and 231), DT-001202 (SEQ ID NOS: 741 and 236), DT-001203 (SEQ ID NOS: 742 and 239), DT-001204 (SEQ ID NOS: 743 and 240), DT-001205 (SEQ ID NOS: 744 and 241), DT-001206 (SEQ ID NOS: 745 and 244), DT-001207 (SEQ ID NOS: 746 and 245), DT-001208 (SEQ ID NOS: 747 and 246), DT-001217 (SEQ ID NOS: 752 and 881), DT-001218 (SEQ ID NOS: 753 and 882), DT-001219 (SEQ ID NOS: 754 and 883), DT-001220 (SEQ ID NOS: 755 and 884), DT-001221 (SEQ ID NOS: 752 and 885), DT-001222 (SEQ ID NOS: 753 and 886), DT-001223 (SEQ ID NOS: 754 and 887), DT-001224 (SEQ ID NOS: 755 and 888), DT-001230 (SEQ ID NOS: 761 and 261), DT-001231 (SEQ ID NOS: 762 and 263), DT-001232 (SEQ ID NOS: 763 and 269), DT-001233 (SEQ ID NOS: 764 and 271), DT-001234 (SEQ ID NOS: 765 and 889), DT-001235 (SEQ ID NOS: 766 and 890), DT-001236 (SEQ ID NOS: 767 and 891), DT-001237 (SEQ ID NOS: 768 and 892), DT-001238 (SEQ ID NOS: 769 and 893), DT-001239 (SEQ ID NOS: 765 and 894), DT-001240 (SEQ ID NOS: 766 and 895), DT-001241 (SEQ ID NOS: 767 and 896), DT-001242 (SEQ ID NOS: 768 and 897), DT-001243 (SEQ ID NOS: 769 and 898), DT-001246 (SEQ ID NOS: 770 and 899), DT-001247 (SEQ ID NOS: 771 and 900), DT-001250 (SEQ ID NOS: 772 and 879), DT-001251 (SEQ ID NOS: 773 and 901), DT-001252 (SEQ ID NOS: 774 and 902), DT-001253 (SEQ ID NOS: 775 and 902), DT-001254 (SEQ ID NOS: 776 and 879), DT-001255 (SEQ ID NOS: 777 and 879), DT-001256 (SEQ ID NOS: 778 and 879), DT-001257 (SEQ ID NOS: 779 and 879), DT-001261 (SEQ ID NOS: 780 and 903), DT-001262 (SEQ ID NOS: 781 and 904), DT-001263 (SEQ ID NOS: 782 and 905), DT-001264 (SEQ ID NOS: 783 and 906), DT-001265 (SEQ ID NOS: 784 and 907), DT-001266 (SEQ ID NOS: 785 and 908), DT-001267 (SEQ ID NOS: 786 and 909), DT-001276 (SEQ ID NOS: 795 and 910), DT-001277 (SEQ ID NOS: 796 and 911), DT-001278 (SEQ ID NOS: 797 and 912), DT-001279 (SEQ ID NOS: 798 and 913), DT-001280 (SEQ ID NOS: 799 and 914), DT-001281 (SEQ ID NOS: 780 and 915), DT-001282 (SEQ ID NOS: 781 and 916), DT-001283 (SEQ ID NOS: 782 and 917), DT-001296 (SEQ ID NOS: 812 and 918), DT-001297 (SEQ ID NOS: 813 and 919), DT-001298 (SEQ ID NOS: 814 and 920), DT-001299 (SEQ ID NOS: 783 and 921), DT-001300 (SEQ ID NOS: 815 and 922), DT-001301 (SEQ ID NOS: 816 and 923), DT-001302 (SEQ ID NOS: 817 and 924), DT-001303 (SEQ ID NOS: 818 and 925), DT-001304 (SEQ ID NOS: 784 and 926), DT-001305 (SEQ ID NOS: 785 and 927), DT-001306 SE ID NOS: 819 and 928), DT-001307 (SEQ ID NOS: 820 and 929), DT-001322 (SEQ ID NOS: 835 and 930), DT-001323 (SEQ ID NOS: 836 and 931), DT-001324 (SEQ ID NOS: 837 and 932), DT-001325 (SEQ ID NOS: 838 and 933), DT-001326 (SEQ ID NOS: 839 and 934), DT-001327 (SEQ ID NOS: 840 and 935), DT-001328 (SEQ ID NOS: 841 and 936), DT-001329 SE ID NOS: 842 and 937), DT-001330 (SEQ ID NOS: 843 and 938), DT-001331 (SEQ ID NOS: 844 and 939), DT-001332 (SEQ ID NOS: 845 and 940), DT-001333 (SEQ ID NOS: 846 and 941), DT-001334 (SEQ ID NOS: 847 and 942), DT-001335 (SEQ ID NOS: 848 and 943), DT-001344 (SEQ ID NOS: 857 and 944), DT-001345 (SEQ ID NOS: 858 and 945), DT-001346 SE ID NOS: 859 and 946), DT-001347 (SEQ ID NOS: 860 and 947), DT-001348 (SEQ ID NOS: 861 and 948), DT-001349 (SEQ ID NOS: 862 and 949), DT-001350 (SEQ ID NOS: 863 and 950), DT-001351 (SEQ ID NOS: 864 and 951), DT-001355 (SEQ ID NOS: 868 and 952), DT-001356 (SEQ ID NOS: 869 and 953), DT-001357 (SEQ ID NOS: 870 and 954), DT-001358 SE ID NOS: 813 and 955), DT-001359 (SEQ ID NOS: 814 and 956), DT-001360 (SEQ ID NOS: 816 and 957), DT-001361 (SEQ ID NOS: 817 and 958), DT-001362 (SEQ ID NOS: 818 and 959), DT-001363 (SEQ ID NOS: 820 and 960), DT-001364 (SEQ ID NOS: 835 and 961), DT-001365 (SEQ ID NOS: 836 and 962), DT-001366 (SEQ ID NOS: 837 and 963), DT-001367 SE ID NOS: 838 and 964), DT-001368 (SEQ ID NOS: 841 and 965), and DT-001369 (SEQ ID NOS: 843 and 966).

145-156. (canceled)

157. The compound of claim 1, or a pharmaceutical salt thereof, wherein the compound is present as a pharmaceutical salt.

158-161. (canceled)

162. A pharmaceutical composition comprising the compound of claim 1 or a pharmaceutical salt thereof.

163. A method of inhibiting the expression of peripheral myelin protein 22 (PMP22) mRNA in a cell, comprising contacting the cell with a compound of claim 1 or a pharmaceutical salt thereof, thereby inhibiting the expression of PMP22 mRNA in the cell.

164-166. (canceled)

167. A method of inhibiting the expression of peripheral myelin protein 22 (PMP22) mRNA in a subject, comprising administering to the subject an effective amount of a compound of claim 1 or a pharmaceutical salt thereof, thereby inhibiting the expression of peripheral myelin protein 22 (PMP22) mRNA.

168-169. (canceled)

170. A method for increasing myelination and/or slowing the loss of myelination in a subject, comprising administering to the subject an effective amount of a compound of claim 1 or a pharmaceutical salt thereof.

171-176. (canceled)

177. A method of treating Charcot-Marie-Tooth disease (CMT), comprising administering to a subject in need thereof an effective amount of a compound of claim 1 or a pharmaceutical salt thereof.

178-194. (canceled)

195. The compound of claim 1, or a pharmaceutical salt thereof, wherein the pharmaceutical salt is an ammonium, potassium, sodium, calcium, or magnesium salt.

196. The compound of claim 195, or a pharmaceutical salt thereof, wherein the pharmaceutical salt is a sodium salt.


SEQUENCE LISTING (1170 sequences)

SEQ ID NO: 1 - DNA, 21 bp, synthetic construct
  misc_feature 1..19: note = RNA
  misc_feature 20..21: note = DNA
  source 1..21: mol_type = other DNA
ctcctcctgttgctgagtatt

SEQ ID NO: 2 - DNA, 21 bp, synthetic construct
  misc_feature 1..19: note = RNA
  misc_feature 20..21: note = DNA
  source 1..21: mol_type = other DNA
ccaatggatcgtgggcaattt

SEQ ID NO: 3 - DNA, 21 bp, synthetic construct
  misc_feature 1..19: note = RNA
  misc_feature 20..21: note = DNA
  source 1..21: mol_type = other DNA
caactgatctctggcagaatt

SEQ ID NO: 4 - DNA, 21 bp, synthetic construct
  misc_feature 1..19: note = RNA
  misc_feature 20..21: note = DNA
  source 1..21: mol_type = other DNA
cacctcttcctcaggaaattt

SEQ ID NO: 5 - DNA, 21 bp, synthetic construct
  misc_feature 1..19: note = RNA
  misc_feature 20..21: note = DNA
  source 1..21: mol_type = other DNA
ggaaatgtccaccactgtttt

SEQ ID NO: 6 - DNA, 21 bp, synthetic construct
  misc_feature 1..19: note = RNA
  misc_feature 20..21: note = DNA
  source 1..21: mol_type = other DNA
gtttctcatcatcaccaaatt

SEQ ID NO: 7 - DNA, 21 bp, synthetic construct
  misc_feature 1..19: note = RNA
  misc_feature 20..21: note = DNA
  source 1..21: mol_type = other DNA
gtcgatcatcttcagcatttt

SEQ ID NO: 8 - DNA, 21 bp, synthetic construct
  misc_feature 1..19: note = RNA
  misc_feature 20..21: note = DNA
  source 1..21: mol_type = other DNA
gttcctgttcttctgccaatt

SEQ ID NO: 9 - DNA, 21 bp, synthetic construct
  misc_feature 1..19: note = RNA
  misc_feature 20..21: note = DNA
  source 1..21: mol_type = other DNA
atcactggaatcttccaaatt

SEQ ID NO: 10 - DNA, 21 bp, synthetic construct
  misc_feature 1..19: note = RNA
  misc_feature 20..21: note = DNA
  source 1..21: mol_type = other DNA
tggaatcttccaaattctttt

SEQ ID NO: 11 - DNA, 21 bp, synthetic construct
  misc_feature 1..19: note = RNA
  misc_feature 20..21: note = DNA
  source 1..21: mol_type = other DNA
ggcatctcaactcggattatt

SEQ ID NO: 12 - DNA, 21 bp, synthetic construct
  misc_feature 1..19: note = RNA
  misc_feature 20..21: note = DNA
  source 1..21: mol_type = other DNA
actcctacggtttcgcctatt

SEQ ID NO: 13 - DNA, 21 bp, synthetic construct
  misc_feature 1..19: note = RNA
  misc_feature 20..21: note = DNA
  source 1..21: mol_type = other DNA
actgatctctggcagaacttt

SEQ ID NO: 14 - DNA, 21 bp, synthetic construct
  misc_feature 1..19: note = RNA
  misc_feature 20..21: note = DNA
  source 1..21: mol_type = other DNA
ctgatctctggcagaactgtt

SEQ ID NO: 15 - DNA, 21 bp, synthetic construct
  misc_feature 1..19: note = RNA
  misc_feature 20..21: note = DNA
  source 1..21: mol_type = other DNA
tgatctctggcagaactgttt

SEQ ID NO: 16 - DNA, 21 bp, synthetic construct
  misc_feature 1..19: note = RNA
  misc_feature 20..21: note = DNA
  source 1..21: mol_type = other DNA
gatctctggcagaactgtatt

SEQ ID NO: 17 - DNA, 21 bp, synthetic construct
  misc_feature 1..19: note = RNA
  misc_feature 20..21: note = DNA
  source 1..21: mol_type = other DNA
agtctgtccaggccaccattt

SEQ ID NO: 18 - DNA, 21 bp, synthetic construct
  misc_feature 1..19: note = RNA
  misc_feature 20..21: note = DNA
  source 1..21: mol_type = other DNA
tctgtccaggccaccatgatt

SEQ ID NO: 19 - DNA, 21 bp, synthetic construct
  misc_feature 1..19: note = RNA
  misc_feature 20..21: note = DNA
  source 1..21: mol_type = other DNA
ctgtccaggccaccatgattt

SEQ ID NO: 20 - DNA, 21 bp, synthetic construct
  misc_feature 1..19: note = RNA
  misc_feature 20..21: note = DNA
  source 1..21: mol_type = other DNA
caggccaccatgatcctgttt

SEQ ID NO: 21 - DNA, 21 bp, synthetic construct
  misc_feature 1..19: note = RNA
  misc_feature 20..21: note = DNA
  source 1..21: mol_type = other DNA
ctctgttcctgttcttctgtt

SEQ ID NO: 22 - DNA, 21 bp, synthetic construct
  misc_feature 1..19: note = RNA
  misc_feature 20..21: note = DNA
  source 1..21: mol_type = other DNA
tctgttcctgttcttctgctt

SEQ ID NO: 23 - DNA, 21 bp, synthetic construct
  misc_feature 1..19: note = RNA
  misc_feature 20..21: note = DNA
  source 1..21: mol_type = other DNA
ttgctggtctgtgcgtgattt

SEQ ID NO: 24 - DNA, 21 bp, synthetic construct
  misc_feature 1..19: note = RNA
  misc_feature 20..21: note = DNA
  source 1..21: mol_type = other DNA
tggtctgtgcgtgatgagttt

SEQ ID NO: 25 - DNA, 21 bp, synthetic construct
  misc_feature 1..19: note = RNA
  misc_feature 20..21: note = DNA
  source 1..21: mol_type = other DNA
tcctgttgctgagtatcattt

SEQ ID NO: 26 - DNA, 21 bp, synthetic construct
  misc_feature 1..19: note = RNA
  misc_feature 20..21: note = DNA
  source 1..21: mol_type = other DNA
cgatcgtcagccaatggattt

SEQ ID NO: 27 - DNA, 21 bp, synthetic construct
  misc_feature 1..19: note = RNA
  misc_feature 20..21: note = DNA
  source 1..21: mol_type = other DNA
cagaactgtagcacctctttt

SEQ ID NO: 28 - DNA, 21 bp, synthetic construct
  misc_feature 1..19: note = RNA
  misc_feature 20..21: note = DNA
  source 1..21: mol_type = other DNA
cctcttcctcaggaaatgttt

SEQ ID NO: 29 - DNA, 21 bp, synthetic construct
  misc_feature 1..19: note = RNA
  misc_feature 20..21: note = DNA
  source 1..21: mol_type = other DNA
tcatcatcaccaaacgaattt

SEQ ID NO: 30 - DNA, 21 bp, synthetic construct
  misc_feature 1..19: note = RNA
  misc_feature 20..21: note = DNA
  source 1..21: mol_type = other DNA
ccatgatcctgtcgatcattt

SEQ ID NO: 31 - DNA, 21 bp, synthetic construct
  misc_feature 1..19: note = RNA
  misc_feature 20..21: note = DNA
  source 1..21: mol_type = other DNA
tcagcattctgtctctgtttt

SEQ ID NO: 32 - DNA, 21 bp, synthetic construct
  misc_feature 1..19: note = RNA
  misc_feature 20..21: note = DNA
  source 1..21: mol_type = other DNA
tctctgttcctgttcttcttt

SEQ ID NO: 33 - DNA, 21 bp, synthetic construct
  misc_feature 1..19: note = RNA
  misc_feature 20..21: note = DNA
  source 1..21: mol_type = other DNA
tcactggaatcttccaaattt

SEQ ID NO: 34 - DNA, 21 bp, synthetic construct
  misc_feature 1..19: note = RNA
  misc_feature 20..21: note = DNA
  source 1..21: mol_type = other DNA
ctggaatcttccaaattcttt

SEQ ID NO: 35 - DNA, 21 bp, synthetic construct
  misc_feature 1..19: note = RNA
  misc_feature 20..21: note = DNA
  source 1..21: mol_type = other DNA
ttctcagcggtgtcatctatt

SEQ ID NO: 36 - DNA, 21 bp, synthetic construct
  misc_feature 1..19: note = RNA
  misc_feature 20..21: note = DNA
  source 1..21: mol_type = other DNA
tctcagcggtgtcatctattt

SEQ ID NO: 37 - DNA, 21 bp, synthetic construct
  misc_feature 1..19: note = RNA
  misc_feature 20..21: note = DNA
  source 1..21: mol_type = other DNA
gctcctcctgttgctgagttt

SEQ ID NO: 38 - DNA, 21 bp, synthetic construct
  misc_feature 1..19: note = RNA
  misc_feature 20..21: note = DNA
  source 1..21: mol_type = other DNA
tcctcctgttgctgagtattt

SEQ ID NO: 39 - DNA, 21 bp, synthetic construct
  misc_feature 1..19: note = RNA
  misc_feature 20..21: note = DNA
  source 1..21: mol_type = other DNA
cctcctgttgctgagtatctt

SEQ ID NO: 40 - DNA, 21 bp, synthetic construct
  misc_feature 1..19: note = RNA
  misc_feature 20..21: note = DNA
  source 1..21: mol_type = other DNA
ctcctgttgctgagtatcatt

SEQ ID NO: 41 - DNA, 21 bp, synthetic construct
  misc_feature 1..19: note = RNA
  misc_feature 20..21: note = DNA
  source 1..21: mol_type = other DNA
cctgttgctgagtatcatctt

SEQ ID NO: 42 - DNA, 21 bp, synthetic construct
  misc_feature 1..19: note = RNA
  misc_feature 20..21: note = DNA
  source 1..21: mol_type = other DNA
ctgttgctgagtatcatcgtt

SEQ ID NO: 43 - DNA, 21 bp, synthetic construct
  misc_feature 1..19: note = RNA
  misc_feature 20..21: note = DNA
  source 1..21: mol_type = other DNA
tgttgctgagtatcatcgttt

SEQ ID NO: 44 - DNA, 21 bp, synthetic construct
  misc_feature 1..19: note = RNA
  misc_feature 20..21: note = DNA
  source 1..21: mol_type = other DNA
gttgctgagtatcatcgtctt

SEQ ID NO: 45 - DNA, 21 bp, synthetic construct
  misc_feature 1..19: note = RNA
  misc_feature 20..21: note = DNA
  source 1..21: mol_type = other DNA
ttgctgagtatcatcgtcctt

SEQ ID NO: 46 - DNA, 21 bp, synthetic construct
  misc_feature 1..19: note = RNA
  misc_feature 20..21: note = DNA
  source 1..21: mol_type = other DNA
tgagtatcatcgtcctccatt

SEQ ID NO: 47 - DNA, 21 bp, synthetic construct
  misc_feature 1..19: note = RNA
  misc_feature 20..21: note = DNA
  source 1..21: mol_type = other DNA
gtatcatcgtcctccacgttt

SEQ ID NO: 48 - DNA, 21 bp, synthetic construct
  misc_feature 1..19: note = RNA
  misc_feature 20..21: note = DNA
  source 1..21: mol_type = other DNA
tcgcggtgctggtgctgcttt

SEQ ID NO: 49 - DNA, 21 bp, synthetic construct
  misc_feature 1..19: note = RNA
  misc_feature 20..21: note = DNA
  source 1..21: mol_type = other DNA
cggtgctggtgctgctgtttt

SEQ ID NO: 50 - DNA, 21 bp, synthetic construct
  misc_feature 1..19: note = RNA
  misc_feature 20..21: note = DNA
  source 1..21: mol_type = other DNA
ctggtgctgctgttcgtcttt

SEQ ID NO: 51 - DNA, 21 bp, synthetic construct
  misc_feature 1..19: note = RNA
  misc_feature 20..21: note = DNA
  source 1..21: mol_type = other DNA
gtgctgctgttcgtctccatt

SEQ ID NO: 52 - DNA, 21 bp, synthetic construct
  misc_feature 1..19: note = RNA
  misc_feature 20..21: note = DNA
  source 1..21: mol_type = other DNA
tgctgttcgtctccacgattt

SEQ ID NO: 53 - DNA, 21 bp, synthetic construct
  misc_feature 1..19: note = RNA
  misc_feature 20..21: note = DNA
  source 1..21: mol_type = other DNA
ttcgtctccacgatcgtcatt

SEQ ID NO: 54 - DNA, 21 bp, synthetic construct
  misc_feature 1..19: note = RNA
  misc_feature 20..21: note = DNA
  source 1..21: mol_type = other DNA
tccacgatcgtcagccaattt

SEQ ID NO: 55 - DNA, 21 bp, synthetic construct
  misc_feature 1..19: note = RNA
  misc_feature 20..21: note = DNA
  source 1..21: mol_type = other DNA
gccaatggatcgtgggcaatt

SEQ ID NO: 56 - DNA, 21 bp, synthetic construct
  misc_feature 1..19: note = RNA
  misc_feature 20..21: note = DNA
  source 1..21: mol_type = other DNA
gtgggcaatggacacgcaatt

SEQ ID NO: 57 - DNA, 21 bp, synthetic construct
  misc_feature 1..19: note = RNA
  misc_feature 20..21: note = DNA
  source 1..21: mol_type = other DNA
caatggacacgcaactgattt

SEQ ID NO: 58 - DNA, 21 bp, synthetic construct
  misc_feature 1..19: note = RNA
  misc_feature 20..21: note = DNA
  source 1..21: mol_type = other DNA
ggacacgcaactgatctcttt

SEQ ID NO: 59 - DNA, 21 bp, synthetic construct
  misc_feature 1..19: note = RNA
  misc_feature 20..21: note = DNA
  source 1..21: mol_type = other DNA
gcacctcttcctcaggaaatt

SEQ ID NO: 60 - DNA, 21 bp, synthetic construct
  misc_feature 1..19: note = RNA
  misc_feature 20..21: note = DNA
  source 1..21: mol_type = other DNA
gaaatgtccaccactgttttt

SEQ ID NO: 61 - DNA, 21 bp, synthetic construct
  misc_feature 1..19: note = RNA
  misc_feature 20..21: note = DNA
  source 1..21: mol_type = other DNA
caccactgtttctcatcattt

SEQ ID NO: 62 - DNA, 21 bp, synthetic construct
  misc_feature 1..19: note = RNA
  misc_feature 20..21: note = DNA
  source 1..21: mol_type = other DNA
tgtttctcatcatcaccaatt

SEQ ID NO: 63 - DNA, 21 bp, synthetic construct
  misc_feature 1..19: note = RNA
  misc_feature 20..21: note = DNA
  source 1..21: mol_type = other DNA
catcatcaccaaacgaatgtt

SEQ ID NO: 64 - DNA, 21 bp, synthetic construct
  misc_feature 1..19: note = RNA
  misc_feature 20..21: note = DNA
  source 1..21: mol_type = other DNA
acgaatggctgcagtctgttt

SEQ ID NO: 65 - DNA, 21 bp, synthetic construct
  misc_feature 1..19: note = RNA
  misc_feature 20..21: note = DNA
  source 1..21: mol_type = other DNA
ccaccatgatcctgtcgattt

SEQ ID NO: 66 - DNA, 21 bp, synthetic construct
  misc_feature 1..19: note = RNA
  misc_feature 20..21: note = DNA
  source 1..21: mol_type = other DNA
tgatcctgtcgatcatctttt

SEQ ID NO: 67 - DNA, 21 bp, synthetic construct
  misc_feature 1..19: note = RNA
  misc_feature 20..21: note = DNA
  source 1..21: mol_type = other DNA
tgtcgatcatcttcagcattt

SEQ ID NO: 68 - DNA, 21 bp, synthetic construct
  misc_feature 1..19: note = RNA
  misc_feature 20..21: note = DNA
  source 1..21: mol_type = other DNA
tcctgttcttctgccaacttt

SEQ ID NO: 69 - DNA, 21 bp, synthetic construct
  misc_feature 1..19: note = RNA
  misc_feature 20..21: note = DNA
  source 1..21: mol_type = other DNA
tgttcttctgccaactctttt

SEQ ID NO: 70 - DNA, 21 bp, synthetic construct
  misc_feature 1..19: note = RNA
  misc_feature 20..21: note = DNA
  source 1..21: mol_type = other DNA
tgccaactcttcaccctcatt

SEQ ID NO: 71 - DNA, 21 bp, synthetic construct
  misc_feature 1..19: note = RNA
  misc_feature 20..21: note = DNA
  source 1..21: mol_type = other DNA
aactcttcaccctcaccaatt

SEQ ID NO: 72 - DNA, 21 bp, synthetic construct
  misc_feature 1..19: note = RNA
  misc_feature 20..21: note = DNA
  source 1..21: mol_type = other DNA
gggcaggttttacatcacttt

SEQ ID NO: 73 - DNA, 21 bp, synthetic construct
  misc_feature 1..19: note = RNA
  misc_feature 20..21: note = DNA
  source 1..21: mol_type = other DNA
ggttttacatcactggaattt

SEQ ID NO: 74 - DNA, 21 bp, synthetic construct
  misc_feature 1..19: note = RNA
  misc_feature 20..21: note = DNA
  source 1..21: mol_type = other DNA
tttacatcactggaatctttt

SEQ ID NO: 75 - DNA, 21 bp, synthetic construct
  misc_feature 1..19: note = RNA
  misc_feature 20..21: note = DNA
  source 1..21: mol_type = other DNA
cactggaatcttccaaatttt

SEQ ID NO: 76 - DNA, 21 bp, synthetic construct
  misc_feature 1..19: note = RNA
  misc_feature 20..21: note = DNA
  source 1..21: mol_type = other DNA
aatcttccaaattcttgcttt

SEQ ID NO: 77 - DNA, 21 bp, synthetic construct
  misc_feature 1..19: note = RNA
  misc_feature 20..21: note = DNA
  source 1..21: mol_type = other DNA
caaattcttgctggtctgttt

SEQ ID NO: 78 - DNA, 21 bp, synthetic construct
  misc_feature 1..19: note = RNA
  misc_feature 20..21: note = DNA
  source 1..21: mol_type = other DNA
gctggtctgtgcgtgatgatt

SEQ ID NO: 79 - DNA, 21 bp, synthetic construct
  misc_feature 1..19: note = RNA
  misc_feature 20..21: note = DNA
  source 1..21: mol_type = other DNA
tgatgagtgctgcggccattt

SEQ ID NO: 80 - DNA, 21 bp, synthetic construct
  misc_feature 1..19: note = RNA
  misc_feature 20..21: note = DNA
  source 1..21: mol_type = other DNA
tgagtgctgcggccatctatt

SEQ ID NO: 81 - DNA, 21 bp, synthetic construct
  misc_feature 1..19: note = RNA
  misc_feature 20..21: note = DNA
  source 1..21: mol_type = other DNA
gcggccatctacacggtgatt

SEQ ID NO: 82 - DNA, 21 bp, synthetic construct
  misc_feature 1..19: note = RNA
  misc_feature 20..21: note = DNA
  source 1..21: mol_type = other DNA
acggtgaggcacccggagttt

SEQ ID NO: 83 - DNA, 21 bp, synthetic construct
  misc_feature 1..19: note = RNA
  misc_feature 20..21: note = DNA
  source 1..21: mol_type = other DNA
gaggcacccggagtggcattt

SEQ ID NO: 84 - DNA, 21 bp, synthetic construct
  misc_feature 1..19: note = RNA
  misc_feature 20..21: note = DNA
  source 1..21: mol_type = other DNA
acccggagtggcatctcaatt

SEQ ID NO: 85 - DNA, 21 bp, synthetic construct
  misc_feature 1..19: note = RNA
  misc_feature 20..21: note = DNA
  source 1..21: mol_type = other DNA
tggcatctcaactcggatttt

SEQ ID NO: 86 - DNA, 21 bp, synthetic construct
  misc_feature 1..19: note = RNA
  misc_feature 20..21: note = DNA
  source 1..21: mol_type = other DNA
tcaactcggattactcctatt

SEQ ID NO: 87 - DNA, 21 bp, synthetic construct
  misc_feature 1..19: note = RNA
  misc_feature 20..21: note = DNA
  source 1..21: mol_type = other DNA
cggattactcctacggttttt

SEQ ID NO: 88 - DNA, 21 bp, synthetic construct
  misc_feature 1..19: note = RNA
  misc_feature 20..21: note = DNA
  source 1..21: mol_type = other DNA
cctacggtttcgcctacattt

SEQ ID NO: 89 - DNA, 21 bp, synthetic construct
  misc_feature 1..19: note = RNA
  misc_feature 20..21: note = DNA
  source 1..21: mol_type = other DNA
acggtttcgcctacatccttt

SEQ ID NO: 90 - DNA, 21 bp, synthetic construct
  misc_feature 1..19: note = RNA
  misc_feature 20..21: note = DNA
  source 1..21: mol_type = other DNA
tcctggcctgggtggcctttt

SEQ ID NO: 91 - DNA, 21 bp, synthetic construct
  misc_feature 1..19: note = RNA
  misc_feature 20..21: note = DNA
  source 1..21: mol_type = other DNA
cccttctcagcggtgtcattt

SEQ ID NO: 92 - DNA, 21 bp, synthetic construct
  misc_feature 1..19: note = RNA
  misc_feature 20..21: note = DNA
  source 1..21: mol_type = other DNA
gcggtgtcatctatgtgattt

SEQ ID NO: 93 - DNA, 21 bp, synthetic construct
  misc_feature 1..19: note = RNA
  misc_feature 20..21: note = DNA
  source 1..21: mol_type = other DNA
gtgtcatctatgtgatctttt

SEQ ID NO: 94 - DNA, 21 bp, synthetic construct
  misc_feature 1..19: note = RNA
  misc_feature 20..21: note = DNA
  source 1..21: mol_type = other DNA
ctatgtgatcttgcggaaatt

SEQ ID NO: 95 - DNA, 21 bp, synthetic construct
  misc_feature 1..19: note = RNA
  misc_feature 20..21: note = DNA
  source 1..21: mol_type = other DNA
atcttgcggaaacgcgaattt

SEQ ID NO: 96 - DNA, 21 bp, synthetic construct
  misc_feature 1..19: note = RNA
  misc_feature 20..21: note = DNA
  source 1..21: mol_type = other DNA
gaggctctgagcgtacatatt

SEQ ID NO: 97 - DNA, 21 bp, synthetic construct
  misc_feature 1..19: note = RNA
  misc_feature 20..21: note = DNA
  source 1..21: mol_type = other DNA
gaggaagggaaaacagaaatt

SEQ ID NO: 98 - DNA, 21 bp, synthetic construct
  misc_feature 1..19: note = RNA
  misc_feature 20..21: note = DNA
  source 1..21: mol_type = other DNA
cccaaaatcccaaactcaatt

SEQ ID NO: 99 - DNA, 21 bp, synthetic construct
  misc_feature 1..19: note = RNA
  misc_feature 20..21: note = DNA
  source 1..21: mol_type = other DNA
atcccaaactcaaaccaaatt

SEQ ID NO: 100 - DNA, 21 bp, synthetic construct
  misc_feature 1..19: note = RNA
  misc_feature 20..21: note = DNA
  source 1..21: mol_type = other DNA
gctgttgattgaagatgtatt

SEQ ID NO: 101 - DNA, 21 bp, synthetic construct
  misc_feature 1..19: note = RNA
  misc_feature 20..21: note = DNA
  source 1..21: mol_type = other DNA
ctgttgattgaagatgtattt

SEQ ID NO: 102 - DNA, 21 bp, synthetic construct
  misc_feature 1..19: note = RNA
  misc_feature 20..21: note = DNA
  source 1..21: mol_type = other DNA
tccggtttataaaacctattt

SEQ ID NO: 103 - DNA, 21 bp, synthetic construct
  misc_feature 1..19: note = RNA
  misc_feature 20..21: note = DNA
  source 1..21: mol_type = other DNA
gtttataaaacctatttattt

SEQ ID NO: 104 - DNA, 21 bp, synthetic construct
  misc_feature 1..19: note = RNA
  misc_feature 20..21: note = DNA
  source 1..21: mol_type = other DNA
acatagtattgtttgcttttt

SEQ ID NO: 105 - DNA, 21 bp, synthetic construct
  misc_feature 1..19: note = RNA
  misc_feature 20..21: note = DNA
  source 1..21: mol_type = other DNA
tgaccatcagcctcgtgtttt

SEQ ID NO: 106 - DNA, 21 bp, synthetic construct
  misc_feature 1..19: note = RNA
  misc_feature 20..21: note = DNA
  source 1..21: mol_type = other DNA
gccttaaagaagtagctaatt

SEQ ID NO: 107 - DNA, 21 bp, synthetic construct
  misc_feature 1..19: note = RNA
  misc_feature 20..21: note = DNA
  source 1..21: mol_type = other DNA
gaagtagctaaggaacttttt

SEQ ID NO: 108 - DNA, 21 bp, synthetic construct
  misc_feature 1..19: note = RNA
  misc_feature 20..21: note = DNA
  source 1..21: mol_type = other DNA
aaggaactttacatcctaatt

SEQ ID NO: 109 - DNA, 21 bp, synthetic construct
  misc_feature 1..19: note = RNA
  misc_feature 20..21: note = DNA
  source 1..21: mol_type = other DNA
ttacatcctaacagtataatt

SEQ ID NO: 110 - DNA, 21 bp, synthetic construct
  misc_feature 1..19: note = RNA
  misc_feature 20..21: note = DNA
  source 1..21: mol_type = other DNA
tacatcctaacagtataattt

SEQ ID NO: 111 - DNA, 21 bp, synthetic construct
  misc_feature 1..19: note = RNA
  misc_feature 20..21: note = DNA
  source 1..21: mol_type = other DNA
ttacccagaaataagataatt

SEQ ID NO: 112 - DNA, 21 bp, synthetic construct
  misc_feature 1..19: note = RNA
  misc_feature 20..21: note = DNA
  source 1..21: mol_type = other DNA
cccttccctttcatctgaatt

SEQ ID NO: 113 - DNA, 21 bp, synthetic construct
  misc_feature 1..19: note = RNA
  misc_feature 20..21: note = DNA
  source 1..21: mol_type = other DNA
ccagtgcatccaacagaaatt

SEQ ID NO: 114 - DNA, 21 bp, synthetic construct
  misc_feature 1..19: note = RNA
  misc_feature 20..21: note = DNA
  source 1..21: mol_type = other DNA
acctctgtgtgaagctttatt

SEQ ID NO: 115 - DNA, 21 bp, synthetic construct
  misc_feature 1..19: note = RNA
  misc_feature 20..21: note = DNA
  source 1..21: mol_type = other DNA
ccaccaactgtagatgtattt

SEQ ID NO: 116 - DNA, 21 bp, synthetic construct
  misc_feature 1..19: note = RNA
  misc_feature 20..21: note = DNA
  source 1..21: mol_type = other DNA
ctgatgctaagactccagatt

SEQ ID NO: 117 - DNA, 21 bp, synthetic construct
  misc_feature 1..19: note = RNA
  misc_feature 20..21: note = DNA
  source 1..21: mol_type = other DNA
tgctttgcattttctgatttt

SEQ ID NO: 118 - DNA, 21 bp, synthetic construct
  misc_feature 1..19: note = RNA
  misc_feature 20..21: note = DNA
  source 1..21: mol_type = other DNA
taccaactgtgtggactaatt

SEQ ID NO: 119 - DNA, 21 bp, synthetic construct
  misc_feature 1..19: note = RNA
  misc_feature 20..21: note = DNA
  source 1..21: mol_type = other DNA
caactgtgtggactaagattt

SEQ ID NO: 120 - DNA, 21 bp, synthetic construct
  misc_feature 1..19: note = RNA
  misc_feature 20..21: note = DNA
  source 1..21: mol_type = other DNA
tgtgtggactaagatgcattt

SEQ ID NO: 121 - DNA, 21 bp, synthetic construct
  misc_feature 1..19: note = RNA
  misc_feature 20..21: note = DNA
  source 1..21: mol_type = other DNA
agggagcaccaccagggaatt

SEQ ID NO: 122 - DNA, 21 bp, synthetic construct
  misc_feature 1..19: note = RNA
  misc_feature 20..21: note = DNA
  source 1..21: mol_type = other DNA
gagcaccaccagggaacattt

SEQ ID NO: 123 - DNA, 21 bp, synthetic construct
  misc_feature 1..19: note = RNA
  misc_feature 20..21: note = DNA
  source 1..21: mol_type = other DNA
agcctggttggaagctgcatt

SEQ ID NO: 124 - DNA, 21 bp, synthetic construct
  misc_feature 1..19: note = RNA
  misc_feature 20..21: note = DNA
  source 1..21: mol_type = other DNA
gttggaagctgcaggcttatt

SEQ ID NO: 125 - DNA, 21 bp, synthetic construct
  misc_feature 1..19: note = RNA
  misc_feature 20..21: note = DNA
  source 1..21: mol_type = other DNA
tggaagctgcaggcttagttt

SEQ ID NO: 126 - DNA, 21 bp, synthetic construct
  misc_feature 1..19: note = RNA
  misc_feature 20..21: note = DNA
  source 1..21: mol_type = other DNA
agctgcaggcttagtctgttt

SEQ ID NO: 127 - DNA, 21 bp, synthetic construct
  misc_feature 1..19: note = RNA
  misc_feature 20..21: note = DNA
  source 1..21: mol_type = other DNA
gggtctctgactgccctgttt

SEQ ID NO: 128 - DNA, 21 bp, synthetic construct
  misc_feature 1..19: note = RNA
  misc_feature 20..21: note = DNA
  source 1..21: mol_type = other DNA
gagggtcttgccttaacattt

SEQ ID NO: 129 - DNA, 21 bp, synthetic construct
  misc_feature 1..19: note = RNA
  misc_feature 20..21: note = DNA
  source 1..21: mol_type = other DNA
tcttgccttaacatccctttt

SEQ ID NO: 130 - DNA, 21 bp, synthetic construct
  misc_feature 1..19: note = RNA
  misc_feature 20..21: note = DNA
  source 1..21: mol_type = other DNA
gccttaacatcccttgcattt

SEQ ID NO: 131 - DNA, 21 bp, synthetic construct
  misc_feature 1..19: note = RNA
  misc_feature 20..21: note = DNA
  source 1..21: mol_type = other DNA
cttaacatcccttgcattttt

SEQ ID NO: 132 - DNA, 21 bp, synthetic construct
  misc_feature 1..19: note = RNA
  misc_feature 20..21: note = DNA
  source 1..21: mol_type = other DNA
cccttgcatttggctgcaatt

SEQ ID NO: 133 - DNA, 21 bp, synthetic construct
  misc_feature 1..19: note = RNA
  misc_feature 20..21: note = DNA
  source 1..21: mol_type = other DNA
ccttgcatttggctgcaaatt

SEQ ID NO: 134 - DNA, 21 bp, synthetic construct
  misc_feature 1..19: note = RNA
  misc_feature 20..21: note = DNA
  source 1..21: mol_type = other DNA
tgcatttggctgcaaagaatt

SEQ ID NO: 135 - DNA, 21 bp, synthetic construct
  misc_feature 1..19: note = RNA
  misc_feature 20..21: note = DNA
  source 1..21: mol_type = other DNA
gcatttggctgcaaagaaatt

SEQ ID NO: 136 - DNA, 21 bp, synthetic construct
  misc_feature 1..19: note = RNA
  misc_feature 20..21: note = DNA
  source 1..21: mol_type = other DNA
catttggctgcaaagaaattt

SEQ ID NO: 137 - DNA, 21 bp, synthetic construct
  misc_feature 1..19: note = RNA
  misc_feature 20..21: note = DNA
  source 1..21: mol_type = other DNA
gctgcaaagaaatctgctttt

SEQ ID NO: 138 - DNA, 21 bp, synthetic construct
  misc_feature 1..19: note = RNA
  misc_feature 20..21: note = DNA
  source 1..21: mol_type = other DNA
caaagaaatctgcttggaatt

SEQ ID NO: 139 - DNA, 21 bp, synthetic construct
  misc_feature 1..19: note = RNA
  misc_feature 20..21: note = DNA
  source 1..21: mol_type = other DNA
agaaatctgcttggaagaatt

SEQ ID NO: 140 - DNA, 21 bp, synthetic construct
  misc_feature 1..19: note = RNA
  misc_feature 20..21: note = DNA
  source 1..21: mol_type = other DNA
tgcttggaagaaggggttatt

SEQ ID NO: 141 - DNA, 21 bp, synthetic construct
  misc_feature 1..19: note = RNA
  misc_feature 20..21: note = DNA
  source 1..21: mol_type = other DNA
ctgtttggccgggcagaaatt

SEQ ID NO: 142 - DNA, 21 bp, synthetic construct
  misc_feature 1..19: note = RNA
  misc_feature 20..21: note = DNA
  source 1..21: mol_type = other DNA
gaaactccgctgagcagaatt

SEQ ID NO: 143 - DNA, 21 bp, synthetic construct
  misc_feature 1..19: note = RNA
  misc_feature 20..21: note = DNA
  source 1..21: mol_type = other DNA
actccgctgagcagaactttt

SEQ ID NO: 144 - DNA, 21 bp, synthetic construct
  misc_feature 1..19: note = RNA
  misc_feature 20..21: note = DNA
  source 1..21: mol_type = other DNA
gcagaacttgccgccagaatt

SEQ ID NO: 145 - DNA, 21 bp, synthetic construct
  misc_feature 1..19: note = RNA
  misc_feature 20..21: note = DNA
  source 1..21: mol_type = other DNA
cagaacttgccgccagaattt

SEQ ID NO: 146 - DNA, 21 bp, synthetic construct
  misc_feature 1..19: note = RNA
  misc_feature 20..21: note = DNA
  source 1..21: mol_type = other DNA
ggctctgttcctgttcttctt

SEQ ID NO: 147 - DNA, 21 bp, synthetic construct
  misc_feature 1..19: note = RNA
  misc_feature 20..21: note = DNA
  source 1..21: mol_type = other DNA
acctatttataacactttttt

SEQ ID NO: 148 - DNA, 21 bp, synthetic construct
  misc_feature 1..19: note = RNA
  misc_feature 20..21: note = DNA
  source 1..21: mol_type = other DNA
acaataaataaatctcaaatt

SEQ ID NO: 149 - DNA, 21 bp, synthetic construct
  misc_feature 1..19: note = RNA
  misc_feature 20..21: note = DNA
  source 1..21: mol_type = other DNA
cctcgtgttgaatcttaaatt

SEQ ID NO: 150 - DNA, 21 bp, synthetic construct
  misc_feature 1..19: note = RNA
  misc_feature 20..21: note = DNA
  source 1..21: mol_type = other DNA
ataccaactgtgtggactatt

SEQ ID NO: 151 - DNA, 21 bp, synthetic construct
  misc_feature 1..19: note = RNA
  misc_feature 20..21: note = DNA
  source 1..21: mol_type = other DNA
aaacctatttataacactttt

SEQ ID NO: 152 - DNA, 21 bp, synthetic construct
  misc_feature 1..19: note = RNA
  misc_feature 20..21: note = DNA
  source 1..21: mol_type = other DNA
tactcagcaacaggaggagtt

SEQ ID NO: 153 - DNA, 21 bp, synthetic construct
  misc_feature 1..19: note = RNA
  misc_feature 20..21: note = DNA
  source 1..21: mol_type = other DNA
attgcccacgatccattggtt

SEQ ID NO: 154 - DNA, 21 bp, synthetic construct
  misc_feature 1..19: note = RNA
  misc_feature 20..21: note = DNA
  source 1..21: mol_type = other DNA
ttctgccagagatcagttgtt

SEQ ID NO: 155 - DNA, 21 bp, synthetic construct
  misc_feature 1..19: note = RNA
  misc_feature 20..21: note = DNA
  source 1..21: mol_type = other DNA
atttcctgaggaagaggtgtt

SEQ ID NO: 156 - DNA, 21 bp, synthetic construct
  misc_feature 1..19: note = RNA
  misc_feature 20..21: note = DNA
  source 1..21: mol_type = other DNA
aacagtggtggacatttcctt

SEQ ID NO: 157 - DNA, 21 bp, synthetic construct
  misc_feature 1..19: note = RNA
  misc_feature 20..21: note = DNA
  source 1..21: mol_type = other DNA
tttggtgatgatgagaaactt

SEQ ID NO: 158 - DNA, 21 bp, synthetic construct
  misc_feature 1..19: note = RNA
  misc_feature 20..21: note = DNA
  source 1..21: mol_type = other DNA
aatgctgaagatgatcgactt

SEQ ID NO: 159 - DNA, 21 bp, synthetic construct
  misc_feature 1..19: note = RNA
  misc_feature 20..21: note = DNA
  source 1..21: mol_type = other DNA
ttggcagaagaacaggaactt

SEQ ID NO: 160 - DNA, 21 bp, synthetic construct
  misc_feature 1..19: note = RNA
  misc_feature 20..21: note = DNA
  source 1..21: mol_type = other DNA
tttggaagattccagtgattt

SEQ ID NO: 161 - DNA, 21 bp, synthetic construct
  misc_feature 1..19: note = RNA
  misc_feature 20..21: note = DNA
  source 1..21: mol_type = other DNA
aagaatttggaagattccatt

SEQ ID NO: 162 - DNA, 21 bp, synthetic construct
  misc_feature 1..19: note = RNA
  misc_feature 20..21: note = DNA
  source 1..21: mol_type = other DNA
taatccgagttgagatgcctt

SEQ ID NO: 163 - DNA, 21 bp, synthetic construct
  misc_feature 1..19: note = RNA
  misc_feature 20..21: note = DNA
  source 1..21: mol_type = other DNA
taggcgaaaccgtaggagttt

SEQ ID NO: 164 - DNA, 21 bp, synthetic construct
  misc_feature 1..19: note = RNA
  misc_feature 20..21: note = DNA
  source 1..21: mol_type = other DNA
agttctgccagagatcagttt

SEQ ID NO: 165 - DNA, 21 bp, synthetic construct
  misc_feature 1..19: note = RNA
  misc_feature 20..21: note = DNA
  source 1..21: mol_type = other DNA
cagttctgccagagatcagtt

SEQ ID NO: 166 - DNA, 21 bp, synthetic construct
  misc_feature 1..19: note = RNA
  misc_feature 20..21: note = DNA
  source 1..21: mol_type = other DNA
acagttctgccagagatcatt

SEQ ID NO: 167 - DNA, 21 bp, synthetic construct
  misc_feature 1..19: note = RNA
  misc_feature 20..21: note = DNA
  source 1..21: mol_type = other DNA
tacagttctgccagagatctt

SEQ ID NO: 168 - DNA, 21 bp, synthetic construct
  misc_feature 1..19: note = RNA
  misc_feature 20..21: note = DNA
  source 1..21: mol_type = other DNA
atggtggcctggacagacttt

SEQ ID NO: 169 - DNA, 21 bp, synthetic construct
  misc_feature 1..19: note = RNA
  misc_feature 20..21: note = DNA
  source 1..21: mol_type = other DNA
tcatggtggcctggacagatt

SEQ ID NO: 170 - DNA, 21 bp, synthetic construct
  misc_feature 1..19: note = RNA
  misc_feature 20..21: note = DNA
  source 1..21: mol_type = other DNA
atcatggtggcctggacagtt

SEQ ID NO: 171 - DNA, 21 bp, synthetic construct
  misc_feature 1..19: note = RNA
  misc_feature 20..21: note = DNA
  source 1..21: mol_type = other DNA
acaggatcatggtggcctgtt

SEQ ID NO: 172 - DNA, 21 bp, synthetic construct
  misc_feature 1..19: note = RNA
  misc_feature 20..21: note = DNA
  source 1..21: mol_type = other DNA
cagaagaacaggaacagagtt

SEQ ID NO: 173 - DNA, 21 bp, synthetic construct
  misc_feature 1..19: note = RNA
  misc_feature 20..21: note = DNA
  source 1..21: mol_type = other DNA
gcagaagaacaggaacagatt

SEQ ID NO: 174 - DNA, 21 bp, synthetic construct
  misc_feature 1..19: note = RNA
  misc_feature 20..21: note = DNA
  source 1..21: mol_type = other DNA
atcacgcacagaccagcaatt

SEQ ID NO: 175 - DNA, 21 bp, synthetic construct
  misc_feature 1..19: note = RNA
  misc_feature 20..21: note = DNA
  source 1..21: mol_type = other DNA
actcatcacgcacagaccatt

SEQ ID NO: 176 - DNA, 21 bp, synthetic construct
  misc_feature 1..19: note = RNA
  misc_feature 20..21: note = DNA
  source 1..21: mol_type = other DNA
atgatactcagcaacaggatt

SEQ ID NO: 177 - DNA, 21 bp, synthetic construct
  misc_feature 1..19: note = RNA
  misc_feature 20..21: note = DNA
  source 1..21: mol_type = other DNA
atccattggctgacgatcgtt

SEQ ID NO: 178 - DNA, 21 bp, synthetic construct
  misc_feature 1..19: note = RNA
  misc_feature 20..21: note = DNA
  source 1..21: mol_type = other DNA
aagaggtgctacagttctgtt

SEQ ID NO: 179 - DNA, 21 bp, synthetic construct
  misc_feature 1..19: note = RNA
  misc_feature 20..21: note = DNA
  source 1..21: mol_type = other DNA
acatttcctgaggaagaggtt

SEQ ID NO: 180 - DNA, 21 bp, synthetic construct
  misc_feature 1..19: note = RNA
  misc_feature 20..21: note = DNA
  source 1..21: mol_type = other DNA
attcgtttggtgatgatgatt

SEQ ID NO: 181 - DNA, 21 bp, synthetic construct
  misc_feature 1..19: note = RNA
  misc_feature 20..21: note = DNA
  source 1..21: mol_type = other DNA
atgatcgacaggatcatggtt

SEQ ID NO: 182 - DNA, 21 bp, synthetic construct
  misc_feature 1..19: note = RNA
  misc_feature 20..21: note = DNA
  source 1..21: mol_type = other DNA
aacagagacagaatgctgatt

SEQ ID NO: 183 - DNA, 21 bp, synthetic construct
  misc_feature 1..19: note = RNA
  misc_feature 20..21: note = DNA
  source 1..21: mol_type = other DNA
agaagaacaggaacagagatt

SEQ ID NO: 184 - DNA, 21 bp, synthetic construct
  misc_feature 1..19: note = RNA
  misc_feature 20..21: note = DNA
  source 1..21: mol_type = other DNA
atttggaagattccagtgatt

SEQ ID NO: 185 - DNA, 21 bp, synthetic construct
  misc_feature 1..19: note = RNA
  misc_feature 20..21: note = DNA
  source 1..21: mol_type = other DNA
agaatttggaagattccagtt

SEQ ID NO: 186 - DNA, 21 bp, synthetic construct
  misc_feature 1..19: note = RNA
  misc_feature 20..21: note = DNA
  source 1..21: mol_type = other DNA
tagatgacaccgctgagaatt

SEQ ID NO: 187 - DNA, 21 bp, synthetic construct
  misc_feature 1..19: note = RNA
  misc_feature 20..21: note = DNA
  source 1..21: mol_type = other DNA
atagatgacaccgctgagatt

SEQ ID NO: 188 - DNA, 21 bp, synthetic construct
  misc_feature 1..19: note = RNA
  misc_feature 20..21: note = DNA
  source 1..21: mol_type = other DNA
atgatactcagcaacaggatt

SEQ ID NO: 189 - DNA, 21 bp, synthetic construct
  misc_feature 1..19: note = RNA
  misc_feature 20..21: note = DNA
  source 1..21: mol_type = other DNA
actcagcaacaggaggagctt

SEQ ID NO: 190 - DNA, 21 bp, synthetic construct
  misc_feature 1..19: note = RNA
  misc_feature 20..21: note = DNA
  source 1..21: mol_type = other DNA
atactcagcaacaggaggatt

SEQ ID NO: 191 - DNA, 21 bp, synthetic construct
  misc_feature 1..19: note = RNA
  misc_feature 20..21: note = DNA
  source 1..21: mol_type = other DNA
gatactcagcaacaggaggtt

SEQ ID NO: 192 - DNA, 21 bp, synthetic construct
  misc_feature 1..19: note = RNA
  misc_feature 20..21: note = DNA
  source 1..21: mol_type = other DNA
tgatactcagcaacaggagtt

SEQ ID NO: 193 - DNA, 21 bp, synthetic construct
  misc_feature 1..19: note = RNA
  misc_feature 20..21: note = DNA
  source 1..21: mol_type = other DNA
gatgatactcagcaacaggtt

SEQ ID NO: 194 - DNA, 21 bp, synthetic construct
  misc_feature 1..19: note = RNA
  misc_feature 20..21: note = DNA
  source 1..21: mol_type = other DNA
cgatgatactcagcaacagtt

SEQ ID NO: 195 - DNA, 21 bp, synthetic construct
  misc_feature 1..19: note = RNA
  misc_feature 20..21: note = DNA
  source 1..21: mol_type = other DNA
acgatgatactcagcaacatt

SEQ ID NO: 196 - DNA, 21 bp, synthetic construct
  misc_feature 1..19: note = RNA
  misc_feature 20..21: note = DNA
  source 1..21: mol_type = other DNA
gacgatgatactcagcaactt

SEQ ID NO: 197 - DNA, 21 bp, synthetic construct
  misc_feature 1..19: note = RNA
  misc_feature 20..21: note = DNA
  source 1..21: mol_type = other DNA
ggacgatgatactcagcaatt

SEQ ID NO: 198 - DNA, 21 bp, synthetic construct
  misc_feature 1..19: note = RNA
  misc_feature 20..21: note = DNA
  source 1..21: mol_type = other DNA
tggaggacgatgatactcatt

SEQ ID NO: 199 - DNA, 21 bp, synthetic construct
  misc_feature 1..19: note = RNA
  misc_feature 20..21: note = DNA
  source 1..21: mol_type = other DNA
acgtggaggacgatgatactt

SEQ ID NO: 200 - DNA, 21 bp, synthetic construct
  misc_feature 1..19: note = RNA
  misc_feature 20..21: note = DNA
  source 1..21: mol_type = other DNA
agcagcaccagcaccgcgatt

SEQ ID NO: 201 - DNA, 21 bp, synthetic construct
  misc_feature 1..19: note = RNA
  misc_feature 20..21: note = DNA
  source 1..21: mol_type = other DNA
aacagcagcaccagcaccgtt

SEQ ID NO: 202 - DNA, 21 bp, synthetic construct
  misc_feature 1..19: note = RNA
  misc_feature 20..21: note = DNA
  source 1..21: mol_type = other DNA
agacgaacagcagcaccagtt

SEQ ID NO: 203 - DNA, 21 bp, synthetic construct
  misc_feature 1..19: note = RNA
  misc_feature 20..21: note = DNA
  source 1..21: mol_type = other DNA
tggagacgaacagcagcactt

SEQ ID NO: 204 - DNA, 21 bp, synthetic construct
  misc_feature 1..19: note = RNA
  misc_feature 20..21: note = DNA
  source 1..21: mol_type = other DNA
atcgtggagacgaacagcatt

SEQ ID NO: 205 - DNA, 21 bp, synthetic construct
  misc_feature 1..19: note = RNA
  misc_feature 20..21: note = DNA
  source 1..21: mol_type = other DNA
tgacgatcgtggagacgaatt

SEQ ID NO: 206 - DNA, 21 bp, synthetic construct
  misc_feature 1..19: note = RNA
  misc_feature 20..21: note = DNA
  source 1..21: mol_type = other DNA
attggctgacgatcgtggatt

SEQ ID NO: 207 - DNA, 21 bp, synthetic construct
  misc_feature 1..19: note = RNA
  misc_feature 20..21: note = DNA
  source 1..21: mol_type = other DNA
ttgcccacgatccattggctt

SEQ ID NO: 208 - DNA, 21 bp, synthetic construct
  misc_feature 1..19: note = RNA
  misc_feature 20..21: note = DNA
  source 1..21: mol_type = other DNA
ttgcgtgtccattgcccactt

SEQ ID NO: 209 - DNA, 21 bp, synthetic construct
  misc_feature 1..19: note = RNA
  misc_feature 20..21: note = DNA
  source 1..21: mol_type = other DNA
atcagttgcgtgtccattgtt

SEQ ID NO: 210 - DNA, 21 bp, synthetic construct
  misc_feature 1..19: note = RNA
  misc_feature 20..21: note = DNA
  source 1..21: mol_type = other DNA
agagatcagttgcgtgtcctt

SEQ ID NO: 211 - DNA, 21 bp, synthetic construct
  misc_feature 1..19: note = RNA
  misc_feature 20..21: note = DNA
  source 1..21: mol_type = other DNA
tttcctgaggaagaggtgctt

SEQ ID NO: 212 - DNA, 21 bp, synthetic construct
  misc_feature 1..19: note = RNA
  misc_feature 20..21: note = DNA
  source 1..21: mol_type = other DNA
aaacagtggtggacatttctt

SEQ ID NO: 213 - DNA, 21 bp, synthetic construct
  misc_feature 1..19: note = RNA
  misc_feature 20..21: note = DNA
  source 1..21: mol_type = other DNA
atgatgagaaacagtggtgtt

SEQ ID NO: 214 - DNA, 21 bp, synthetic construct
  misc_feature 1..19: note = RNA
  misc_feature 20..21: note = DNA
  source 1..21: mol_type = other DNA
ttggtgatgatgagaaacatt

SEQ ID NO: 215 - DNA, 21 bp, synthetic construct
  misc_feature 1..19: note = RNA
  misc_feature 20..21: note = DNA
  source 1..21: mol_type = other DNA
cattcgtttggtgatgatgtt

SEQ ID NO: 216 - DNA, 21 bp, synthetic construct
  misc_feature 1..19: note = RNA
  misc_feature 20..21: note = DNA
  source 1..21: mol_type = other DNA
acagactgcagccattcgttt

SEQ ID NO: 217 - DNA, 21 bp, synthetic construct
  misc_feature 1..19: note = RNA
  misc_feature 20..21: note = DNA
  source 1..21: mol_type = other DNA
atcgacaggatcatggtggtt

SEQ ID NO: 218 - DNA, 21 bp, synthetic construct
  misc_feature 1..19: note = RNA
  misc_feature 20..21: note = DNA
  source 1..21: mol_type = other DNA
aagatgatcgacaggatcatt

SEQ ID NO: 219 - DNA, 21 bp, synthetic construct
  misc_feature 1..19: note = RNA
  misc_feature 20..21: note = DNA
  source 1..21: mol_type = other DNA
atgctgaagatgatcgacatt

SEQ ID NO: 220 - DNA, 21 bp, synthetic construct
  misc_feature 1..19: note = RNA
  misc_feature 20..21: note = DNA
  source 1..21: mol_type = other DNA
agttggcagaagaacaggatt

SEQ ID NO: 221 - DNA, 21 bp, synthetic construct
  misc_feature 1..19: note = RNA
  misc_feature 20..21: note = DNA
  source 1..21: mol_type = other DNA
aagagttggcagaagaacatt

SEQ ID NO: 222 - DNA, 21 bp, synthetic construct
  misc_feature 1..19: note = RNA
  misc_feature 20..21: note = DNA
  source 1..21: mol_type = other DNA
tgagggtgaagagttggcatt

SEQ ID NO: 223 - DNA, 21 bp, synthetic construct
  misc_feature 1..19: note = RNA
  misc_feature 20..21: note = DNA
  source 1..21: mol_type = other DNA
ttggtgagggtgaagagtttt

SEQ ID NO: 224 - DNA, 21 bp, synthetic construct
  misc_feature 1..19: note = RNA
  misc_feature 20..21: note = DNA
  source 1..21: mol_type = other DNA
agtgatgtaaaacctgccctt

SEQ ID NO: 225 - DNA, 21 bp, synthetic construct
  misc_feature 1..19: note = RNA
  misc_feature 20..21: note = DNA
  source 1..21: mol_type = other DNA
attccagtgatgtaaaacctt

SEQ ID NO: 226 - DNA, 21 bp, synthetic construct
  misc_feature 1..19: note = RNA
  misc_feature 20..21: note = DNA
  source 1..21: mol_type = other DNA
aagattccagtgatgtaaatt

SEQ ID NO: 227 - DNA, 21 bp, synthetic construct
  misc_feature 1..19: note = RNA
  misc_feature 20..21: note = DNA
  source 1..21: mol_type = other DNA
aatttggaagattccagtgtt

SEQ ID NO: 228 - DNA, 21 bp, synthetic construct
  misc_feature 1..19: note = RNA
  misc_feature 20..21: note = DNA
  source 1..21: mol_type = other DNA
agcaagaatttggaagatttt

SEQ ID NO: 229 - DNA, 21 bp, synthetic construct
  misc_feature 1..19: note = RNA
  misc_feature 20..21: note = DNA
  source 1..21: mol_type = other DNA
acagaccagcaagaatttgtt

SEQ ID NO: 230 - DNA, 21 bp, synthetic construct
  misc_feature 1..19: note = RNA
  misc_feature 20..21: note = DNA
  source 1..21: mol_type = other DNA
tcatcacgcacagaccagctt

SEQ ID NO: 231 - DNA, 21 bp, synthetic construct
  misc_feature 1..19: note = RNA
  misc_feature 20..21: note = DNA
  source 1..21: mol_type = other DNA
atggccgcagcactcatcatt

SEQ ID NO: 232 - DNA, 21 bp, synthetic construct
  misc_feature 1..19: note = RNA
  misc_feature 20..21: note = DNA
  source 1..21: mol_type = other DNA
tagatggccgcagcactcatt

SEQ ID NO: 233 - DNA, 21 bp, synthetic construct
  misc_feature 1..19: note = RNA
  misc_feature 20..21: note = DNA
  source 1..21: mol_type = other DNA
tcaccgtgtagatggccgctt

SEQ ID NO: 234 - DNA, 21 bp, synthetic construct
  misc_feature 1..19: note = RNA
  misc_feature 20..21: note = DNA
  source 1..21: mol_type = other DNA
actccgggtgcctcaccgttt

SEQ ID NO: 235 - DNA, 21 bp, synthetic construct
  misc_feature 1..19: note = RNA
  misc_feature 20..21: note = DNA
  source 1..21: mol_type = other DNA
atgccactccgggtgcctctt

SEQ ID NO: 236 - DNA, 21 bp, synthetic construct
  misc_feature 1..19: note = RNA
  misc_feature 20..21: note = DNA
  source 1..21: mol_type = other DNA
ttgagatgccactccgggttt

SEQ ID NO: 237 - DNA, 21 bp, synthetic construct
  misc_feature 1..19: note = RNA
  misc_feature 20..21: note = DNA
  source 1..21: mol_type = other DNA
aatccgagttgagatgccatt

SEQ ID NO: 238 - DNA, 21 bp, synthetic construct
  misc_feature 1..19: note = RNA
  misc_feature 20..21: note = DNA
  source 1..21: mol_type = other DNA
taggagtaatccgagttgatt

SEQ ID NO: 239 - DNA, 21 bp, synthetic construct
  misc_feature 1..19: note = RNA
  misc_feature 20..21: note = DNA
  source 1..21: mol_type = other DNA
aaaccgtaggagtaatccgtt

SEQ ID NO: 240 - DNA, 21 bp, synthetic construct
  misc_feature 1..19: note = RNA
  misc_feature 20..21: note = DNA
  source 1..21: mol_type = other DNA
atgtaggcgaaaccgtaggtt

SEQ ID NO: 241 - DNA, 21 bp, synthetic construct
  misc_feature 1..19: note = RNA
  misc_feature 20..21: note = DNA
  source 1..21: mol_type = other DNA
aggatgtaggcgaaaccgttt

SEQ ID NO: 242 - DNA, 21 bp, synthetic construct
  misc_feature 1..19: note = RNA
  misc_feature 20..21: note = DNA
  source 1..21: mol_type = other DNA
aaggccacccaggccaggatt

SEQ ID NO: 243 - DNA, 21 bp, synthetic construct
  misc_feature 1..19: note = RNA
  misc_feature 20..21: note = DNA
  source 1..21: mol_type = other DNA
atgacaccgctgagaagggtt

SEQ ID NO: 244 - DNA, 21 bp, synthetic construct
  misc_feature 1..19: note = RNA
  misc_feature 20..21: note = DNA
  source 1..21: mol_type = other DNA
atcacatagatgacaccgctt

SEQ ID NO: 245 - DNA, 21 bp, synthetic construct
  misc_feature 1..19: note = RNA
  misc_feature 20..21: note = DNA
  source 1..21: mol_type = other DNA
aagatcacatagatgacactt

SEQ ID NO: 246 - DNA, 21 bp, synthetic construct
  misc_feature 1..19: note = RNA
  misc_feature 20..21: note = DNA
  source 1..21: mol_type = other DNA
tttccgcaagatcacatagtt

SEQ ID NO: 247 - DNA, 21 bp, synthetic construct
  misc_feature 1..19: note = RNA
  misc_feature 20..21: note = DNA
  source 1..21: mol_type = other DNA
attcgcgtttccgcaagattt

SEQ ID NO: 248 - DNA, 21 bp, synthetic construct
  misc_feature 1..19: note = RNA
  misc_feature 20..21: note = DNA
  source 1..21: mol_type = other DNA
tatgtacgctcagagcctctt

SEQ ID NO: 249 - DNA, 21 bp, synthetic construct
  misc_feature 1..19: note = RNA
  misc_feature 20..21: note = DNA
  source 1..21: mol_type = other DNA
tttctgttttcccttcctctt

SEQ ID NO: 250 - DNA, 21 bp, synthetic construct
  misc_feature 1..19: note = RNA
  misc_feature 20..21: note = DNA
  source 1..21: mol_type = other DNA
ttgagtttgggattttgggtt

SEQ ID NO: 251 - DNA, 21 bp, synthetic construct
  misc_feature 1..19: note = RNA
  misc_feature 20..21: note = DNA
  source 1..21: mol_type = other DNA
tttggtttgagtttgggattt

SEQ ID NO: 252 - DNA, 21 bp, synthetic construct
  misc_feature 1..19: note = RNA
  misc_feature 20..21: note = DNA
  source 1..21: mol_type = other DNA
tacatcttcaatcaacagctt

SEQ ID NO: 253 - DNA, 21 bp, synthetic construct
  misc_feature 1..19: note = RNA
  misc_feature 20..21: note = DNA
  source 1..21: mol_type = other DNA
atacatcttcaatcaacagtt

SEQ ID NO: 254 - DNA, 21 bp, synthetic construct
  misc_feature 1..19: note = RNA
  misc_feature 20..21: note = DNA
  source 1..21: mol_type = other DNA
ataggttttataaaccggatt

SEQ ID NO: 255 - DNA, 21 bp, synthetic construct
  misc_feature 1..19: note = RNA
  misc_feature 20..21: note = DNA
  source 1..21: mol_type = other DNA
ataaataggttttataaactt

SEQ ID NO: 256 - DNA, 21 bp, synthetic construct
  misc_feature 1..19: note = RNA
  misc_feature 20..21: note = DNA
  source 1..21: mol_type = other DNA
aaagcaaacaatactatgttt

SEQ ID NO: 257 - DNA, 21 bp, synthetic construct
  misc_feature 1..19: note = RNA
  misc_feature 20..21: note = DNA
  source 1..21: mol_type = other DNA
aacacgaggctgatggtcatt

SEQ ID NO: 258 - DNA, 21 bp, synthetic construct
  misc_feature 1..19: note = RNA
  misc_feature 20..21: note = DNA
  source 1..21: mol_type = other DNA
ttagctacttctttaaggctt

SEQ ID NO: 259 - DNA, 21 bp, synthetic construct
  misc_feature 1..19: note = RNA
  misc_feature 20..21: note = DNA
  source 1..21: mol_type = other DNA
aaagttccttagctacttctt

SEQ ID NO: 260 - DNA, 21 bp, synthetic construct
  misc_feature 1..19: note = RNA
  misc_feature 20..21: note = DNA
  source 1..21: mol_type = other DNA
ttaggatgtaaagttcctttt

SEQ ID NO: 261 - DNA, 21 bp, synthetic construct
  misc_feature 1..19: note = RNA
  misc_feature 20..21: note = DNA
  source 1..21: mol_type = other DNA
ttatactgttaggatgtaatt

SEQ ID NO: 262 - DNA, 21 bp, synthetic construct
  misc_feature 1..19: note = RNA
  misc_feature 20..21: note = DNA
  source 1..21: mol_type = other DNA
attatactgttaggatgtatt

SEQ ID NO: 263 - DNA, 21 bp, synthetic construct
  misc_feature 1..19: note = RNA
  misc_feature 20..21: note = DNA
  source 1..21: mol_type = other DNA
ttatcttatttctgggtaatt

SEQ ID NO: 264 - DNA, 21 bp, synthetic construct
  misc_feature 1..19: note = RNA
  misc_feature 20..21: note = DNA
  source 1..21: mol_type = other DNA
ttcagatgaaagggaagggtt

SEQ ID NO: 265 - DNA, 21 bp, synthetic construct
  misc_feature 1..19: note = RNA
  misc_feature 20..21: note = DNA
  source 1..21: mol_type = other DNA
tttctgttggatgcactggtt

SEQ ID NO: 266 - DNA, 21 bp, synthetic construct
  misc_feature 1..19: note = RNA
  misc_feature 20..21: note = DNA
  source 1..21: mol_type = other DNA
taaagcttcacacagaggttt

SEQ ID NO: 267 - DNA, 21 bp, synthetic construct
  misc_feature 1..19: note = RNA
  misc_feature 20..21: note = DNA
  source 1..21: mol_type = other DNA
atacatctacagttggtggtt

SEQ ID NO: 268 - DNA, 21 bp, synthetic construct
  misc_feature 1..19: note = RNA
  misc_feature 20..21: note = DNA
  source 1..21: mol_type = other DNA
tctggagtcttagcatcagtt

SEQ ID NO: 269 - DNA, 21 bp, synthetic construct
  misc_feature 1..19: note = RNA
  misc_feature 20..21: note = DNA
  source 1..21: mol_type = other DNA
aatcagaaaatgcaaagcatt

SEQ ID NO: 270 - DNA, 21 bp, synthetic construct
  misc_feature 1..19: note = RNA
  misc_feature 20..21: note = DNA
  source 1..21: mol_type = other DNA
ttagtccacacagttggtatt

SEQ ID NO: 271 - DNA, 21 bp, synthetic construct
  misc_feature 1..19: note = RNA
  misc_feature 20..21: note = DNA
  source 1..21: mol_type = other DNA
atcttagtccacacagttgtt

SEQ ID NO: 272 - DNA, 21 bp, synthetic construct
  misc_feature 1..19: note = RNA
  misc_feature 20..21: note = DNA
  source 1..21: mol_type = other DNA
atgcatcttagtccacacatt

SEQ ID NO: 273 - DNA, 21 bp, synthetic construct
  misc_feature 1..19: note = RNA
  misc_feature 20..21: note = DNA
  source 1..21: mol_type = other DNA
ttccctggtggtgctcccttt

SEQ ID NO: 274 - DNA, 21 bp, synthetic construct
  misc_feature 1..19: note = RNA
  misc_feature 20..21: note = DNA
  source 1..21: mol_type = other DNA
atgttccctggtggtgctctt

SEQ ID NO: 275 - DNA, 21 bp, synthetic construct
  misc_feature 1..19: note = RNA
  misc_feature 20..21: note = DNA
  source 1..21: mol_type = other DNA
tgcagcttccaaccaggcttt

SEQ ID NO: 276 - DNA, 21 bp, synthetic construct
  misc_feature 1..19: note = RNA
  misc_feature 20..21: note = DNA
  source 1..21: mol_type = other DNA
taagcctgcagcttccaactt

SEQ ID NO: 277 - DNA, 21 bp, synthetic construct
  misc_feature 1..19: note = RNA
  misc_feature 20..21: note = DNA
  source 1..21: mol_type = other DNA
actaagcctgcagcttccatt

SEQ ID NO: 278 - DNA, 21 bp, synthetic construct
  misc_feature 1..19: note = RNA
  misc_feature 20..21: note = DNA
  source 1..21: mol_type = other DNA
acagactaagcctgcagcttt

SEQ ID NO: 279 - DNA, 21 bp, synthetic construct
  misc_feature 1..19: note = RNA
  misc_feature 20..21: note = DNA
  source 1..21: mol_type = other DNA
acagggcagtcagagaccctt

SEQ ID NO: 280 - DNA, 21 bp, synthetic construct
  misc_feature 1..19: note = RNA
  misc_feature 20..21: note = DNA
  source 1..21: mol_type = other DNA
atgttaaggcaagaccctctt

SEQ ID NO: 281 - DNA, 21 bp, synthetic construct
  misc_feature 1..19: note = RNA
  misc_feature 20..21: note = DNA
  source 1..21: mol_type = other DNA
aagggatgttaaggcaagatt

SEQ ID NO: 282 - DNA, 21 bp, synthetic construct
  misc_feature 1..19: note = RNA
  misc_feature 20..21: note = DNA
  source 1..21: mol_type = other DNA
atgcaagggatgttaaggctt

SEQ ID NO: 283 - DNA, 21 bp, synthetic construct
  misc_feature 1..19: note = RNA
  misc_feature 20..21: note = DNA
  source 1..21: mol_type = other DNA
aaatgcaagggatgttaagtt

SEQ ID NO: 284 - DNA, 21 bp, synthetic construct
  misc_feature 1..19: note = RNA
  misc_feature 20..21: note = DNA
  source 1..21: mol_type = other DNA
ttgcagccaaatgcaagggtt

SEQ ID NO: 285 - DNA, 21 bp, synthetic construct
  misc_feature 1..19: note = RNA
  misc_feature 20..21: note = DNA
  source 1..21: mol_type = other DNA
tttgcagccaaatgcaaggtt

SEQ ID NO: 286 - DNA, 21 bp, synthetic construct
  misc_feature 1..19: note = RNA
  misc_feature 20..21: note = DNA
  source 1..21: mol_type = other DNA
ttctttgcagccaaatgcatt

SEQ ID NO: 287 - DNA, 21 bp, synthetic construct
  misc_feature 1..19: note = RNA
  misc_feature 20..21: note = DNA
  source 1..21: mol_type = other DNA
tttctttgcagccaaatgctt

SEQ ID NO: 288 - DNA, 21 bp, synthetic construct
  misc_feature 1..19: note = RNA
  misc_feature 20..21: note = DNA
  source 1..21: mol_type = other DNA
atttctttgcagccaaatgtt

SEQ ID NO: 289 - DNA, 21 bp, synthetic construct
  misc_feature 1..19: note = RNA
  misc_feature 20..21: note = DNA
  source 1..21: mol_type = other DNA
aagcagatttctttgcagctt

SEQ ID NO: 290 - DNA, 21 bp, synthetic construct
  misc_feature 1..19: note = RNA
  misc_feature 20..21: note = DNA
  source 1..21: mol_type = other DNA
ttccaagcagatttctttgtt

SEQ ID NO: 291 - DNA, 21 bp, synthetic construct
  misc_feature 1..19: note = RNA
  misc_feature 20..21: note = DNA
  source 1..21: mol_type = other DNA
ttcttccaagcagatttcttt

SEQ ID NO: 292 - DNA, 21 bp, synthetic construct
  misc_feature 1..19: note = RNA
  misc_feature 20..21: note = DNA
  source 1..21: mol_type = other DNA
taaccccttcttccaagcatt

SEQ ID NO: 293 - DNA, 21 bp, synthetic construct
  misc_feature 1..19: note = RNA
  misc_feature 20..21: note = DNA
  source 1..21: mol_type = other DNA
tttctgcccggccaaacagtt

SEQ ID NO: 294 - DNA, 21 bp, synthetic construct
  misc_feature 1..19: note = RNA
  misc_feature 20..21: note = DNA
  source 1..21: mol_type = other DNA
ttctgctcagcggagtttctt

SEQ ID NO: 295 - DNA, 21 bp, synthetic construct
  misc_feature 1..19: note = RNA
  misc_feature 20..21: note = DNA
  source 1..21: mol_type = other DNA
aagttctgctcagcggagttt

SEQ ID NO: 296 - DNA, 21 bp, synthetic construct
  misc_feature 1..19: note = RNA
  misc_feature 20..21: note = DNA
  source 1..21: mol_type = other DNA
ttctggcggcaagttctgctt

SEQ ID NO: 297 - DNA, 21 bp, synthetic construct
  misc_feature 1..19: note = RNA
  misc_feature 20..21: note = DNA
  source 1..21: mol_type = other DNA
attctggcggcaagttctgtt

SEQ ID NO: 298 - DNA, 21 bp, synthetic construct
  misc_feature 1..19: note = RNA
  misc_feature 20..21: note = DNA
  source 1..21: mol_type = other DNA
aagattccagtgatgtaaatt

SEQ ID NO: 299 - DNA, 21 bp, synthetic construct
  misc_feature 1..19: note = RNA
  misc_feature 20..21: note = DNA
  source 1..21: mol_type = other DNA
ttggtgagggtgaagagtttt

SEQ ID NO: 300 - DNA, 21 bp, synthetic construct
  misc_feature 1..19: note = RNA
  misc_feature 20..21: note = DNA
  source 1..21: mol_type = other DNA
agtgatgtaaaacctgccctt

SEQ ID NO: 301 - DNA, 21 bp, synthetic construct
  misc_feature 1..19: note = RNA
  misc_feature 20..21: note = DNA
  source 1..21: mol_type = other DNA
attccagtgatgtaaaacctt

SEQ ID NO: 302 - DNA, 21 bp, synthetic construct
  misc_feature 1..19: note = RNA
  misc_feature 20..21: note = DNA
  source 1..21: mol_type = other DNA
aatttggaagattccagtgtt

SEQ ID NO: 303 - DNA, 21 bp, synthetic construct
  misc_feature 1..19: note = RNA
  misc_feature 20..21: note = DNA
  source 1..21: mol_type = other DNA
agcaagaatttggaagatttt

SEQ ID NO: 304 - DNA, 21 bp, synthetic construct
  misc_feature 1..19: note = RNA
  misc_feature 20..21: note = DNA
  source 1..21: mol_type = other DNA
acagaccagcaagaatttgtt

SEQ ID NO: 305 - DNA, 21 bp, synthetic construct
  misc_feature 1..19: note = RNA
  misc_feature 20..21: note = DNA
  source 1..21: mol_type = other DNA
tcatcacgcacagaccagctt

SEQ ID NO: 306 - DNA, 21 bp, synthetic construct
  misc_feature 1..19: note = RNA
  misc_feature 20..21: note = DNA
  source 1..21: mol_type = other DNA
atggccgcagcactcatcatt

SEQ ID NO: 307 - DNA, 21 bp, synthetic construct
  misc_feature 1..19: note = RNA
  misc_feature 20..21: note = DNA
  source 1..21: mol_type = other DNA
atgccactccgggtgcctctt

SEQ ID NO: 308 - DNA, 21 bp, synthetic construct
  misc_feature 1..19: note = RNA
  misc_feature 20..21: note = DNA
  source 1..21: mol_type = other DNA
ttgagatgccactccgggttt

SEQ ID NO: 309 - DNA, 21 bp, synthetic construct
  misc_feature 1..19: note = RNA
  misc_feature 20..21: note = DNA
  source 1..21: mol_type = other DNA
taggagtaatccgagttgatt

SEQ ID NO: 310 - DNA, 21 bp, synthetic construct
  misc_feature 1..19: note = RNA
  misc_feature 20..21: note = DNA
  source 1..21: mol_type = other DNA
aaaccgtaggagtaatccgtt

SEQ ID NO: 311 - DNA, 21 bp, synthetic construct
  misc_feature 1..19: note = RNA
  misc_feature 20..21: note = DNA
  source 1..21: mol_type = other DNA
atgtaggcgaaaccgtaggtt

SEQ ID NO: 312 - DNA, 21 bp, synthetic construct
  misc_feature 1..19: note = RNA
  misc_feature 20..21: note = DNA
  source 1..21: mol_type = other DNA
aggatgtaggcgaaaccgttt

SEQ ID NO: 313 - DNA, 21 bp, synthetic construct
  misc_feature 1..19: note = RNA
  misc_feature 20..21: note = DNA
  source 1..21: mol_type = other DNA
atgacaccgctgagaagggtt

SEQ ID NO: 314 - DNA, 21 bp, synthetic construct
  misc_feature 1..19: note = RNA
  misc_feature 20..21: note = DNA
  source 1..21: mol_type = other DNA
atcacatagatgacaccgctt

SEQ ID NO: 315 - DNA, 21 bp, synthetic construct
  misc_feature 1..19: note = RNA
  misc_feature 20..21: note = DNA
  source 1..21: mol_type = other DNA
aagatcacatagatgacactt

SEQ ID NO: 316 - DNA, 21 bp, synthetic construct
  misc_feature 1..19: note = RNA
  misc_feature 20..21: note = DNA
  source 1..21: mol_type = other DNA
tttccgcaagatcacatagtt

SEQ ID NO: 317 - DNA, 21 bp, synthetic construct
  misc_feature 1..19: note = RNA
  misc_feature 20..21: note = DNA
  source 1..21: mol_type = other DNA
gaagaacaggaacagagcctt

SEQ ID NO: 318 - DNA, 21 bp, synthetic construct
  misc_feature 1..19: note = RNA
  misc_feature 20..21: note = DNA
  source 1..21: mol_type = other DNA
aaaagtgttataaataggttt

SEQ ID NO: 319 - DNA, 21 bp, synthetic construct
  misc_feature 1..19: note = RNA
  misc_feature 20..21: note = DNA
  source 1..21: mol_type = other DNA
tttgagatttatttattgttt

SEQ ID NO: 320 - DNA, 21 bp, synthetic construct
  misc_feature 1..19: note = RNA
  misc_feature 20..21: note = DNA
  source 1..21: mol_type = other DNA
tttaagattcaacacgaggtt

SEQ ID NO: 321 - DNA, 21 bp, synthetic construct
  misc_feature 1..19: note = RNA
  misc_feature 20..21: note = DNA
  source 1..21: mol_type = other DNA
tagtccacacagttggtattt

SEQ ID NO: 322 - DNA, 21 bp, synthetic construct
  misc_feature 1..19: note = RNA
  misc_feature 20..21: note = DNA
  source 1..21: mol_type = other DNA
aagtgttataaataggttttt

SEQ ID NO: 323 - DNA, 21 bp, synthetic construct
  misc_feature 1..19: note = RNA
  misc_feature 20..21: note = DNA
  source 1..21: mol_type = other DNA
tttctgttttcccttcctctt

SEQ ID NO: 324 - DNA, 21 bp, synthetic construct
  misc_feature 1..19: note = RNA
  misc_feature 20..21: note = DNA
  source 1..21: mol_type = other DNA
ttgagtttgggattttgggtt

SEQ ID NO: 325 - DNA, 21 bp, synthetic construct
  misc_feature 1..19: note = RNA
  misc_feature 20..21: note = DNA
  source 1..21: mol_type = other DNA
tttggtttgagtttgggattt

SEQ ID NO: 326 - DNA, 21 bp, synthetic construct
  misc_feature 1..19: note = RNA
  misc_feature 20..21: note = DNA
  source 1..21: mol_type = other DNA
tacatcttcaatcaacagctt

SEQ ID NO: 327 - DNA, 21 bp, synthetic construct
  misc_feature 1..19: note = RNA
  misc_feature 20..21: note = DNA
  source 1..21: mol_type = other DNA
atacatcttcaatcaacagtt

SEQ ID NO: 328 - DNA, 21 bp, synthetic construct
  misc_feature 1..19: note = RNA
  misc_feature 20..21: note = DNA
  source 1..21: mol_type = other DNA
ataaataggttttataaactt

SEQ ID NO: 329 - DNA, 21 bp, synthetic construct
  misc_feature 1..19: note = RNA
  misc_feature 20..21: note = DNA
  source 1..21: mol_type = other DNA
aaagcaaacaatactatgttt

SEQ ID NO: 330 - DNA, 21 bp, synthetic construct
  misc_feature 1..19: note = RNA
  misc_feature 20..21: note = DNA
  source 1..21: mol_type = other DNA
aacacgaggctgatggtcatt

SEQ ID NO: 331 - DNA, 21 bp, synthetic construct
  misc_feature 1..19: note = RNA
  misc_feature 20..21: note = DNA
  source 1..21: mol_type = other DNA
ttagctacttctttaaggctt

SEQ ID NO: 332 - DNA, 21 bp, synthetic construct
  misc_feature 1..19: note = RNA
  misc_feature 20..21: note = DNA
  source 1..21: mol_type = other DNA
aaagttccttagctacttctt

SEQ ID NO: 333 - DNA, 21 bp, synthetic construct
  misc_feature 1..19: note = RNA
  misc_feature 20..21: note = DNA
  source 1..21: mol_type = other DNA
ttaggatgtaaagttcctttt

SEQ ID NO: 334 - DNA, 21 bp, synthetic construct
  misc_feature 1..19: note = RNA
  misc_feature 20..21: note = DNA
  source 1..21: mol_type = other DNA
atgcatcttagtccacacatt

SEQ ID NO: 335 - DNA, 21 bp, synthetic construct
  misc_feature 1..19: note = RNA
  misc_feature 20..21: note = DNA
  source 1..21: mol_type = other DNA
cgatgatactcagcaacagtt

SEQ ID NO: 336 - DNA, 21 bp, synthetic construct
  misc_feature 1..19: note = RNA
  misc_feature 20..21: note = DNA
  source 1..21: mol_type = other DNA
gacgatgatactcagcaactt

SEQ ID NO: 337 - DNA, 21 bp, synthetic construct
  misc_feature 1..19: note = RNA
  misc_feature 20..21: note = DNA
  source 1..21: mol_type = other DNA
acgtggaggacgatgatactt

SEQ ID NO: 338 - DNA, 21 bp, synthetic construct
  misc_feature 1..19: note = RNA
  misc_feature 20..21: note = DNA
  source 1..21: mol_type = other DNA
agcagcaccagcaccgcgatt

SEQ ID NO: 339 - DNA, 21 bp, synthetic construct
  misc_feature 1..19: note = RNA
  misc_feature 20..21: note = DNA
  source 1..21: mol_type = other DNA
agacgaacagcagcaccagtt

SEQ ID NO: 340 - DNA, 21 bp, synthetic construct
  misc_feature 1..19: note = RNA
  misc_feature 20..21: note = DNA
  source 1..21: mol_type = other DNA
actcctacggtttcgcctatt

SEQ ID NO: 341 - DNA, 21 bp, synthetic construct
  misc_feature 1..19: note = RNA
  misc_feature 20..21: note = DNA
  source 1..21: mol_type = other DNA
actgatctctggcagaacttt

SEQ ID NO: 342 - DNA, 21 bp, synthetic construct
  misc_feature 1..19: note = RNA
  misc_feature 20..21: note = DNA
  source 1..21: mol_type = other DNA
agtctgtccaggccaccattt

SEQ ID NO: 343 - DNA, 21 bp, synthetic construct
  misc_feature 1..19: note = RNA
  misc_feature 20..21: note = DNA
  source 1..21: mol_type = other DNA
atcactggaatcttccaaatt

SEQ ID NO: 344 - DNA, 21 bp, synthetic construct
  misc_feature 1..19: note = RNA
  misc_feature 20..21: note = DNA
  source 1..21: mol_type = other DNA
caactgatctctggcagaatt

SEQ ID NO: 345 - DNA, 21 bp, synthetic construct
  misc_feature 1..19: note = RNA
  misc_feature 20..21: note = DNA
  source 1..21: mol_type = other DNA
cacctcttcctcaggaaattt

SEQ ID NO: 346 - DNA, 21 bp, synthetic construct
  misc_feature 1..19: note = RNA
  misc_feature 20..21: note = DNA
  source 1..21: mol_type = other DNA
cagaactgtagcacctctttt

SEQ ID NO: 347 - DNA, 21 bp, synthetic construct
  misc_feature 1..19: note = RNA
  misc_feature 20..21: note = DNA
  source 1..21: mol_type = other DNA
caggccaccatgatcctgttt

SEQ ID NO: 348 - DNA, 21 bp, synthetic construct
  misc_feature 1..19: note = RNA
  misc_feature 20..21: note = DNA
  source 1..21: mol_type = other DNA
ccaatggatcgtgggcaattt

SEQ ID NO: 349 - DNA, 21 bp, synthetic construct
  misc_feature 1..19: note = RNA
  misc_feature 20..21: note = DNA
  source 1..21: mol_type = other DNA
ccatgatcctgtcgatcattt

SEQ ID NO: 350 - DNA, 21 bp, synthetic construct
  misc_feature 1..19: note = RNA
  misc_feature 20..21: note = DNA
  source 1..21: mol_type = other DNA
cctcttcctcaggaaatgttt

SEQ ID NO: 351 - DNA, 21 bp, synthetic construct
  misc_feature 1..19: note = RNA
  misc_feature 20..21: note = DNA
  source 1..21: mol_type = other DNA
cgatcgtcagccaatggattt

SEQ ID NO: 352 - DNA, 21 bp, synthetic construct
  misc_feature 1..19: note = RNA
  misc_feature 20..21: note = DNA
  source 1..21: mol_type = other DNA
ctcctcctgttgctgagtatt

SEQ ID NO: 353 - DNA, 21 bp, synthetic construct
  misc_feature 1..19: note = RNA
  misc_feature 20..21: note = DNA
  source 1..21: mol_type = other DNA
ctctgttcctgttcttctgtt

SEQ ID NO: 354 - DNA, 21 bp, synthetic construct
  misc_feature 1..19: note = RNA
  misc_feature 20..21: note = DNA
  source 1..21: mol_type = other DNA
ctgatctctggcagaactgtt

SEQ ID NO: 355 - DNA, 21 bp, synthetic construct
  misc_feature 1..19: note = RNA
  misc_feature 20..21: note = DNA
  source 1..21: mol_type = other DNA
ctggaatcttccaaattcttt

SEQ ID NO: 356 - DNA, 21 bp, synthetic construct
  misc_feature 1..19: note = RNA
  misc_feature 20..21: note = DNA
  source 1..21: mol_type = other DNA
ctgtccaggccaccatgattt

SEQ ID NO: 357 - DNA, 21 bp, synthetic construct
  misc_feature 1..19: note = RNA
  misc_feature 20..21: note = DNA
  source 1..21: mol_type = other DNA
gatctctggcagaactgtatt

SEQ ID NO: 358 - DNA, 21 bp, synthetic construct
  misc_feature 1..19: note = RNA
  misc_feature 20..21: note = DNA
  source 1..21: mol_type = other DNA
ggaaatgtccaccactgtttt

SEQ ID NO: 359 - DNA, 21 bp, synthetic construct
  misc_feature 1..19: note = RNA
  misc_feature 20..21: note = DNA
  source 1..21: mol_type = other DNA
ggcatctcaactcggattatt

SEQ ID NO: 360 - DNA, 21 bp, synthetic construct
  misc_feature 1..19: note = RNA
  misc_feature 20..21: note = DNA
  source 1..21: mol_type = other DNA
gtcgatcatcttcagcatttt

SEQ ID NO: 361 - DNA, 21 bp, synthetic construct
  misc_feature 1..19: note = RNA
  misc_feature 20..21: note = DNA
  source 1..21: mol_type = other DNA
gttcctgttcttctgccaatt

SEQ ID NO: 362 - DNA, 21 bp, synthetic construct
  misc_feature 1..19: note = RNA
  misc_feature 20..21: note = DNA
  source 1..21: mol_type = other DNA
gtttctcatcatcaccaaatt

SEQ ID NO: 363 - DNA, 21 bp, synthetic construct
  misc_feature 1..19: note = RNA
  misc_feature 20..21: note = DNA
  source 1..21: mol_type = other DNA
tcactggaatcttccaaattt

SEQ ID NO: 364 - DNA, 21 bp, synthetic construct
  misc_feature 1..19: note = RNA
  misc_feature 20..21: note = DNA
  source 1..21: mol_type = other DNA
tcagcattctgtctctgtttt

SEQ ID NO: 365 - DNA, 21 bp, synthetic construct
  misc_feature 1..19: note = RNA
  misc_feature 20..21: note = DNA
  source 1..21: mol_type = other DNA
tcatcatcaccaaacgaattt

SEQ ID NO: 366 - DNA, 21 bp, synthetic construct
  misc_feature 1..19: note = RNA
  misc_feature 20..21: note = DNA
  source 1..21: mol_type = other DNA
tcctgttgctgagtatcattt

SEQ ID NO: 367 - DNA, 21 bp, synthetic construct
  misc_feature 1..19: note = RNA
  misc_feature 20..21: note = DNA
  source 1..21: mol_type = other DNA
tctcagcggtgtcatctattt

SEQ ID NO: 368 - DNA, 21 bp, synthetic construct
  misc_feature 1..19: note = RNA
  misc_feature 20..21: note = DNA
  source 1..21: mol_type = other DNA
tctctgttcctgttcttcttt

SEQ ID NO: 369 - DNA, 21 bp, synthetic construct
  misc_feature 1..19: note = RNA
  misc_feature 20..21: note = DNA
  source 1..21: mol_type = other DNA
tctgtccaggccaccatgatt

SEQ ID NO: 370 - DNA, 21 bp, synthetic construct
  misc_feature 1..19: note = RNA
  misc_feature 20..21: note = DNA
  source 1..21: mol_type = other DNA
tctgttcctgttcttctgctt

SEQ ID NO: 371 - DNA, 21 bp, synthetic construct
  misc_feature 1..19: note = RNA
  misc_feature 20..21: note = DNA
  source 1..21: mol_type = other DNA
tgatctctggcagaactgttt

SEQ ID NO: 372 - DNA, 21 bp, synthetic construct
  misc_feature 1..19: note = RNA
  misc_feature 20..21: note = DNA
  source 1..21: mol_type = other DNA
tggaatcttccaaattctttt

SEQ ID NO: 373 - DNA, 21 bp, synthetic construct
  misc_feature 1..19: note = RNA
  misc_feature 20..21: note = DNA
  source 1..21: mol_type = other DNA
tggtctgtgcgtgatgagttt

SEQ ID NO: 374 - DNA, 21 bp, synthetic construct
  misc_feature 1..19: note = RNA
  misc_feature 20..21: note = DNA
  source 1..21: mol_type = other DNA
ttctcagcggtgtcatctatt

SEQ ID NO: 375 - DNA, 21 bp, synthetic construct
  misc_feature 1..19: note = RNA
  misc_feature 20..21: note = DNA
  source 1..21: mol_type = other DNA
ttgctggtctgtgcgtgattt

SEQ ID NO: 376 - DNA, 21 bp, synthetic construct
  misc_feature 1..19: note = RNA
  misc_feature 20..21: note = DNA
  source 1..21: mol_type = other DNA
gctcctcctgttgctgagttt

SEQ ID NO: 377 - DNA, 21 bp, synthetic construct
  misc_feature 1..19: note = RNA
  misc_feature 20..21: note = DNA
  source 1..21: mol_type = other DNA
tcctcctgttgctgagtattt

SEQ ID NO: 378 - DNA, 21 bp, synthetic construct
  misc_feature 1..19: note = RNA
  misc_feature 20..21: note = DNA
  source 1..21: mol_type = other DNA
cctcctgttgctgagtatctt

SEQ ID NO: 379 - DNA, 21 bp, synthetic construct
  misc_feature 1..19: note = RNA
  misc_feature 20..21: note = DNA
  source 1..21: mol_type = other DNA
ctcctgttgctgagtatcatt

SEQ ID NO: 380 - DNA, 21 bp, synthetic construct
  misc_feature 1..19: note = RNA
  misc_feature 20..21: note = DNA
  source 1..21: mol_type = other DNA
cctgttgctgagtatcatctt

SEQ ID NO: 381 - DNA, 21 bp, synthetic construct
  misc_feature 1..19: note = RNA
  misc_feature 20..21: note = DNA
  source 1..21: mol_type = other DNA
ctgttgctgagtatcatcgtt

SEQ ID NO: 382 - DNA, 21 bp, synthetic construct
  misc_feature 1..19: note = RNA
  misc_feature 20..21: note = DNA
  source 1..21: mol_type = other DNA
tgttgctgagtatcatcgttt

SEQ ID NO: 383 - DNA, 21 bp, synthetic construct
  misc_feature 1..19: note = RNA
  misc_feature 20..21: note = DNA
  source 1..21: mol_type = other DNA
gttgctgagtatcatcgtctt

SEQ ID NO: 384 - DNA, 21 bp, synthetic construct
  misc_feature 1..19: note = RNA
  misc_feature 20..21: note = DNA
  source 1..21: mol_type = other DNA
ttgctgagtatcatcgtcctt

SEQ ID NO: 385 - DNA, 21 bp, synthetic construct
  misc_feature 1..19: note = RNA
  misc_feature 20..21: note = DNA
  source 1..21: mol_type = other DNA
tgagtatcatcgtcctccatt

SEQ ID NO: 386 - DNA, 21 bp, synthetic construct
  misc_feature 1..19: note = RNA
  misc_feature 20..21: note = DNA
  source 1..21: mol_type = other DNA
gtatcatcgtcctccacgttt

SEQ ID NO: 387 - DNA, 21 bp, synthetic construct
  misc_feature 1..19: note = RNA
  misc_feature 20..21: note = DNA
  source 1..21: mol_type = other DNA
tcgcggtgctggtgctgcttt

SEQ ID NO: 388 - DNA, 21 bp, synthetic construct
  misc_feature 1..19: note = RNA
  misc_feature 20..21: note = DNA
  source 1..21: mol_type = other DNA
cggtgctggtgctgctgtttt

SEQ ID NO: 389 - DNA, 21 bp, synthetic construct
  misc_feature 1..19: note = RNA
  misc_feature 20..21: note = DNA
  source 1..21: mol_type = other DNA
ctggtgctgctgttcgtcttt

SEQ ID NO: 390 - DNA, 21 bp, synthetic construct
  misc_feature 1..19: note = RNA
  misc_feature 20..21: note = DNA
  source 1..21: mol_type = other DNA
gtgctgctgttcgtctccatt

SEQ ID NO: 391 - DNA, 21 bp, synthetic construct
  misc_feature 1..19: note = RNA
  misc_feature 20..21: note = DNA
  source 1..21: mol_type = other DNA
tgctgttcgtctccacgattt

SEQ ID NO: 392 - DNA, 21 bp, synthetic construct
  misc_feature 1..19: note = RNA
  misc_feature 20..21: note = DNA
  source 1..21: mol_type = other DNA
ttcgtctccacgatcgtcatt

SEQ ID NO: 393 - DNA, 21 bp, synthetic construct
  misc_feature 1..19: note = RNA
  misc_feature 20..21: note = DNA
  source 1..21: mol_type = other DNA
tccacgatcgtcagccaattt

SEQ ID NO: 394 - DNA, 21 bp, synthetic construct
  misc_feature 1..19: note = RNA
  misc_feature 20..21: note = DNA
  source 1..21: mol_type = other DNA
gccaatggatcgtgggcaatt

SEQ ID NO: 395 - DNA, 21 bp, synthetic construct
  misc_feature 1..19: note = RNA
  misc_feature 20..21: note = DNA
  source 1..21: mol_type = other DNA
gtgggcaatggacacgcaatt

SEQ ID NO: 396 - DNA, 21 bp, synthetic construct
  misc_feature 1..19: note = RNA
  misc_feature 20..21: note = DNA
  source 1..21: mol_type = other DNA
caatggacacgcaactgattt

SEQ ID NO: 397 - DNA, 21 bp, synthetic construct
  misc_feature 1..19: note = RNA
  misc_feature 20..21: note = DNA
  source 1..21: mol_type = other DNA
ggacacgcaactgatctcttt

SEQ ID NO: 398 - DNA, 21 bp, synthetic construct
  misc_feature 1..19: note = RNA
  misc_feature 20..21: note = DNA
  source 1..21: mol_type = other DNA
gcacctcttcctcaggaaatt

SEQ ID NO: 399 - DNA, 21 bp, synthetic construct
  misc_feature 1..19: note = RNA
  misc_feature 20..21: note = DNA
  source 1..21: mol_type = other DNA
gaaatgtccaccactgttttt

SEQ ID NO: 400 - DNA, 21 bp, synthetic construct
  misc_feature 1..19: note = RNA
  misc_feature 20..21: note = DNA
  source 1..21: mol_type = other DNA
caccactgtttctcatcattt

SEQ ID NO: 401 - DNA, 21 bp, synthetic construct
  misc_feature 1..19: note = RNA
  misc_feature 20..21: note = DNA
  source 1..21: mol_type = other DNA
tgtttctcatcatcaccaatt

SEQ ID NO: 402 - DNA, 21 bp, synthetic construct
  misc_feature 1..19: note = RNA
  misc_feature 20..21: note = DNA
  source 1..21: mol_type = other DNA
catcatcaccaaacgaatgtt

SEQ ID NO: 403 - DNA, 21 bp, synthetic construct
  misc_feature 1..19: note = RNA
  misc_feature 20..21: note = DNA
  source 1..21: mol_type = other DNA
acgaatggctgcagtctgttt

SEQ ID NO: 404 - DNA, 21 bp, synthetic construct
  misc_feature 1..19: note = RNA
  misc_feature 20..21: note = DNA
  source 1..21: mol_type = other DNA
ccaccatgatcctgtcgattt

SEQ ID NO: 405 - DNA, 21 bp, synthetic construct
  misc_feature 1..19: note = RNA
  misc_feature 20..21: note = DNA
  source 1..21: mol_type = other DNA
tgatcctgtcgatcatctttt

SEQ ID NO: 406 - DNA, 21 bp, synthetic construct
  misc_feature 1..19: note = RNA
  misc_feature 20..21: note = DNA
  source 1..21: mol_type = other DNA
tgtcgatcatcttcagcattt

SEQ ID NO: 407 - DNA, 21 bp, synthetic construct
  misc_feature 1..19: note = RNA
  misc_feature 20..21: note = DNA
  source 1..21: mol_type = other DNA
tcctgttcttctgccaacttt

SEQ ID NO: 408 - DNA, 21 bp, synthetic construct
  misc_feature 1..19: note = RNA
  misc_feature 20..21: note = DNA
  source 1..21: mol_type = other DNA
tgttcttctgccaactctttt

SEQ ID NO: 409 - DNA, 21 bp, synthetic construct
  misc_feature 1..19: note = RNA
  misc_feature 20..21: note = DNA
  source 1..21: mol_type = other DNA
tgccaactcttcaccctcatt

SEQ ID NO: 410 - DNA, 21 bp, synthetic construct
  misc_feature 1..19: note = RNA
  misc_feature 20..21: note = DNA
  source 1..21: mol_type = other DNA
aactcttcaccctcaccaatt

SEQ ID NO: 411 - DNA, 21 bp, synthetic construct
  misc_feature 1..19: note = RNA
  misc_feature 20..21: note = DNA
  source 1..21: mol_type = other DNA
gggcaggttttacatcacttt

SEQ ID NO: 412 - DNA, 21 bp, synthetic construct
  misc_feature 1..19: note = RNA
  misc_feature 20..21: note = DNA
  source 1..21: mol_type = other DNA
ggttttacatcactggaattt

SEQ ID NO: 413 - DNA, 21 bp, synthetic construct
  misc_feature 1..19: note = RNA
  misc_feature 20..21: note = DNA
  source 1..21: mol_type = other DNA
tttacatcactggaatctttt

SEQ ID NO: 414 - DNA, 21 bp, synthetic construct
  misc_feature 1..19: note = RNA
  misc_feature 20..21: note = DNA
  source 1..21: mol_type = other DNA
cactggaatcttccaaatttt

SEQ ID NO: 415 - DNA, 21 bp, synthetic construct
  misc_feature 1..19: note = RNA
  misc_feature 20..21: note = DNA
  source 1..21: mol_type = other DNA
aatcttccaaattcttgcttt

SEQ ID NO: 416 - DNA, 21 bp, synthetic construct
  misc_feature 1..19: note = RNA
  misc_feature 20..21: note = DNA
  source 1..21: mol_type = other DNA
caaattcttgctggtctgttt

SEQ ID NO: 417 - DNA, 21 bp, synthetic construct
  misc_feature 1..19: note = RNA
  misc_feature 20..21: note = DNA
  source 1..21: mol_type = other DNA
gctggtctgtgcgtgatgatt

SEQ ID NO: 418 - DNA, 21 bp, synthetic construct
  misc_feature 1..19: note = RNA
  misc_feature 20..21: note = DNA
  source 1..21: mol_type = other DNA
tgatgagtgctgcggccattt

SEQ ID NO: 419 - DNA, 21 bp, synthetic construct
  misc_feature 1..19: note = RNA
  misc_feature 20..21: note = DNA
  source 1..21: mol_type = other DNA
tgagtgctgcggccatctatt

SEQ ID NO: 420 - DNA, 21 bp, synthetic construct
  misc_feature 1..19: note = RNA
  misc_feature 20..21: note = DNA
  source 1..21: mol_type = other DNA
gcggccatctacacggtgatt

SEQ ID NO: 421 - DNA, 21 bp, synthetic construct
  misc_feature 1..19: note = RNA
  misc_feature 20..21: note = DNA
  source 1..21: mol_type = other DNA
acggtgaggcacccggagttt

SEQ ID NO: 422 - DNA, 21 bp, synthetic construct
  misc_feature 1..19: note = RNA
  misc_feature 20..21: note = DNA
  source 1..21: mol_type = other DNA
gaggcacccggagtggcattt

SEQ ID NO: 423 - DNA, 21 bp, synthetic construct
  misc_feature 1..19: note = RNA
  misc_feature 20..21: note = DNA
  source 1..21: mol_type = other DNA
acccggagtggcatctcaatt

SEQ ID NO: 424 - DNA, 21 bp, synthetic construct
  misc_feature 1..19: note = RNA
  misc_feature 20..21: note = DNA
  source 1..21: mol_type = other DNA
tggcatctcaactcggatttt

SEQ ID NO: 425 - DNA, 21 bp, synthetic construct
  misc_feature 1..19: note = RNA
  misc_feature 20..21: note = DNA
  source 1..21: mol_type = other DNA
tcaactcggattactcctatt

SEQ ID NO: 426 - DNA, 21 bp, synthetic construct
  misc_feature 1..19: note = RNA
  misc_feature 20..21: note = DNA
  source 1..21: mol_type = other DNA
cggattactcctacggttttt

SEQ ID NO: 427 - DNA, 21 bp, synthetic construct
  misc_feature 1..19: note = RNA
  misc_feature 20..21: note = DNA
  source 1..21: mol_type = other DNA
cctacggtttcgcctacattt

SEQ ID NO: 428 - DNA, 21 bp, synthetic construct
  misc_feature 1..19: note = RNA
  misc_feature 20..21: note = DNA
  source 1..21: mol_type = other DNA
acggtttcgcctacatccttt

SEQ ID NO: 429 - DNA, 21 bp, synthetic construct
  misc_feature 1..19: note = RNA
  misc_feature 20..21: note = DNA
  source 1..21: mol_type = other DNA
tcctggcctgggtggcctttt

SEQ ID NO: 430 - DNA, 21 bp, synthetic construct
  misc_feature 1..19: note = RNA
  misc_feature 20..21: note = DNA
  source 1..21: mol_type = other DNA
cccttctcagcggtgtcattt

SEQ ID NO: 431 - DNA, 21 bp, synthetic construct
  misc_feature 1..19: note = RNA
  misc_feature 20..21: note = DNA
  source 1..21: mol_type = other DNA
gcggtgtcatctatgtgattt

SEQ ID NO: 432 - DNA, 21 bp, synthetic construct
  misc_feature 1..19: note = RNA
  misc_feature 20..21: note = DNA
  source 1..21: mol_type = other DNA
gtgtcatctatgtgatctttt

SEQ ID NO: 433 - DNA, 21 bp, synthetic construct
  misc_feature 1..19: note = RNA
  misc_feature 20..21: note = DNA
  source 1..21: mol_type = other DNA
ctatgtgatcttgcggaaatt

SEQ ID NO: 434 - DNA, 21 bp, synthetic construct
  misc_feature 1..19: note = RNA
  misc_feature 20..21: note = DNA
  source 1..21: mol_type = other DNA
atcttgcggaaacgcgaattt

SEQ ID NO: 435 - DNA, 21 bp, synthetic construct
  misc_feature 1..19: note = RNA
  misc_feature 20..21: note = DNA
  source 1..21: mol_type = other DNA
gaggctctgagcgtacatatt

SEQ ID NO: 436 - DNA, 21 bp, synthetic construct
  misc_feature 1..19: note = RNA
  misc_feature 20..21: note = DNA
  source 1..21: mol_type = other DNA
gaggaagggaaaacagaaatt

SEQ ID NO: 437 - DNA, 21 bp, synthetic construct
  misc_feature 1..19: note = RNA
  misc_feature 20..21: note = DNA
  source 1..21: mol_type = other DNA
cccaaaatcccaaactcaatt

SEQ ID NO: 438 - DNA, 21 bp, synthetic construct
  misc_feature 1..19: note = RNA
  misc_feature 20..21: note = DNA
  source 1..21: mol_type = other DNA
atcccaaactcaaaccaaatt

SEQ ID NO: 439 - DNA, 21 bp, synthetic construct
  misc_feature 1..19: note = RNA
  misc_feature 20..21: note = DNA
  source 1..21: mol_type = other DNA
gctgttgattgaagatgtatt

SEQ ID NO: 440 - DNA, 21 bp, synthetic construct
  misc_feature 1..19: note = RNA
  misc_feature 20..21: note = DNA
  source 1..21: mol_type = other DNA
ctgttgattgaagatgtattt

SEQ ID NO: 441 - DNA, 21 bp, synthetic construct
  misc_feature 1..19: note = RNA
  misc_feature 20..21: note = DNA
  source 1..21: mol_type = other DNA
tccggtttataaaacctattt

SEQ ID NO: 442 - DNA, 21 bp, synthetic construct
  misc_feature 1..19: note = RNA
  misc_feature 20..21: note = DNA
  source 1..21: mol_type = other DNA
gtttataaaacctatttattt

SEQ ID NO: 443 - DNA, 21 bp, synthetic construct
  misc_feature 1..19: note = RNA
  misc_feature 20..21: note = DNA
  source 1..21: mol_type = other DNA
acatagtattgtttgcttttt

SEQ ID NO: 444 - DNA, 21 bp, synthetic construct
  misc_feature 1..19: note = RNA
  misc_feature 20..21: note = DNA
  source 1..21: mol_type = other DNA
tgaccatcagcctcgtgtttt

SEQ ID NO: 445 - DNA, 21 bp, synthetic construct
  misc_feature 1..19: note = RNA
  misc_feature 20..21: note = DNA
  source 1..21: mol_type = other DNA
gccttaaagaagtagctaatt

SEQ ID NO: 446 - DNA, 21 bp, synthetic construct
  misc_feature 1..19: note = RNA
  misc_feature 20..21: note = DNA
  source 1..21: mol_type = other DNA
gaagtagctaaggaacttttt

SEQ ID NO: 447 - DNA, 21 bp, synthetic construct
  misc_feature 1..19: note = RNA
  misc_feature 20..21: note = DNA
  source 1..21: mol_type = other DNA
aaggaactttacatcctaatt

SEQ ID NO: 448 - DNA, 21 bp, synthetic construct
  misc_feature 1..19: note = RNA
  misc_feature 20..21: note = DNA
  source 1..21: mol_type = other DNA
ttacatcctaacagtataatt

SEQ ID NO: 449 - DNA, 21 bp, synthetic construct
  misc_feature 1..19: note = RNA
  misc_feature 20..21: note = DNA
  source 1..21: mol_type = other DNA
tacatcctaacagtataattt

SEQ ID NO: 450 - DNA, 21 bp, synthetic construct
  misc_feature 1..19: note = RNA
  misc_feature 20..21: note = DNA
  source 1..21: mol_type = other DNA
ttacccagaaataagataatt

SEQ ID NO: 451 - DNA, 21 bp, synthetic construct
  misc_feature 1..19: note = RNA
  misc_feature 20..21: note = DNA
  source 1..21: mol_type = other DNA
cccttccctttcatctgaatt

SEQ ID NO: 452 - DNA, 21 bp, synthetic construct
  misc_feature 1..19: note = RNA
  misc_feature 20..21: note = DNA
  source 1..21: mol_type = other DNA
ccagtgcatccaacagaaatt

SEQ ID NO: 453 - DNA, 21 bp, synthetic construct
  misc_feature 1..19: note = RNA
  misc_feature 20..21: note = DNA
  source 1..21: mol_type = other DNA
acctctgtgtgaagctttatt

SEQ ID NO: 454 - DNA, 21 bp, synthetic construct
  misc_feature 1..19: note = RNA
  misc_feature 20..21: note = DNA
  source 1..21: mol_type = other DNA
ccaccaactgtagatgtattt

SEQ ID NO: 455 - DNA, 21 bp, synthetic construct
  misc_feature 1..19: note = RNA
  misc_feature 20..21: note = DNA
  source 1..21: mol_type = other DNA
ctgatgctaagactccagatt

SEQ ID NO: 456 - DNA, 21 bp, synthetic construct
  misc_feature 1..19: note = RNA
  misc_feature 20..21: note = DNA
  source 1..21: mol_type = other DNA
tgctttgcattttctgatttt

SEQ ID NO: 457 - DNA, 21 bp, synthetic construct
  misc_feature 1..19: note = RNA
  misc_feature 20..21: note = DNA
  source 1..21: mol_type = other DNA
taccaactgtgtggactaatt

SEQ ID NO: 458 - DNA, 21 bp, synthetic construct
  misc_feature 1..19: note = RNA
  misc_feature 20..21: note = DNA
  source 1..21: mol_type = other DNA
caactgtgtggactaagattt

SEQ ID NO: 459 - DNA, 21 bp, synthetic construct
  misc_feature 1..19: note = RNA
  misc_feature 20..21: note = DNA
  source 1..21: mol_type = other DNA
tgtgtggactaagatgcattt

SEQ ID NO: 460 - DNA, 21 bp, synthetic construct
  misc_feature 1..19: note = RNA
  misc_feature 20..21: note = DNA
  source 1..21: mol_type = other DNA
agggagcaccaccagggaatt

SEQ ID NO: 461 - DNA, 21 bp, synthetic construct
  misc_feature 1..19: note = RNA
  misc_feature 20..21: note = DNA
  source 1..21: mol_type = other DNA
gagcaccaccagggaacattt

SEQ ID NO: 462 - DNA, 21 bp, synthetic construct
  misc_feature 1..19: note = RNA
  misc_feature 20..21: note = DNA
  source 1..21: mol_type = other DNA
agcctggttggaagctgcatt

SEQ ID NO: 463 - DNA, 21 bp, synthetic construct
  misc_feature 1..19: note = RNA
  misc_feature 20..21: note = DNA
  source 1..21: mol_type = other DNA
gttggaagctgcaggcttatt

SEQ ID NO: 464 - DNA, 21 bp, synthetic construct
  misc_feature 1..19: note = RNA
  misc_feature 20..21: note = DNA
  source 1..21: mol_type = other DNA
tggaagctgcaggcttagttt

SEQ ID NO: 465 - DNA, 21 bp, synthetic construct
  misc_feature 1..19: note = RNA
  misc_feature 20..21: note = DNA
  source 1..21: mol_type = other DNA
agctgcaggcttagtctgttt

SEQ ID NO: 466 - DNA, 21 bp, synthetic construct
  misc_feature 1..19: note = RNA
  misc_feature 20..21: note = DNA
  source 1..21: mol_type = other DNA
gggtctctgactgccctgttt

SEQ ID NO: 467 - DNA, 21 bp, synthetic construct
  misc_feature 1..19: note = RNA
  misc_feature 20..21: note = DNA
  source 1..21: mol_type = other DNA
gagggtcttgccttaacattt

SEQ ID NO: 468 - DNA, 21 bp, synthetic construct
  misc_feature 1..19: note = RNA
  misc_feature 20..21: note = DNA
  source 1..21: mol_type = other DNA
tcttgccttaacatccctttt

SEQ ID NO: 469 - DNA, 21 bp, synthetic construct
  misc_feature 1..19: note = RNA
  misc_feature 20..21: note = DNA
  source 1..21: mol_type = other DNA
gccttaacatcccttgcattt

SEQ ID NO: 470 - DNA, 21 bp, synthetic construct
  misc_feature 1..19: note = RNA
  misc_feature 20..21: note = DNA
  source 1..21: mol_type = other DNA
cttaacatcccttgcattttt

SEQ ID NO: 471 - DNA, 21 bp, synthetic construct
  misc_feature 1..19: note = RNA
  misc_feature 20..21: note = DNA
  source 1..21: mol_type = other DNA
cccttgcatttggctgcaatt

SEQ ID NO: 472 - DNA, 21 bp, synthetic construct
  misc_feature 1..19: note = RNA
  misc_feature 20..21: note = DNA
  source 1..21: mol_type = other DNA
ccttgcatttggctgcaaatt

SEQ ID NO: 473 - DNA, 21 bp, synthetic construct
  misc_feature 1..19: note = RNA
  misc_feature 20..21: note = DNA
  source 1..21: mol_type = other DNA
tgcatttggctgcaaagaatt

SEQ ID NO: 474 - DNA, 21 bp, synthetic construct
  misc_feature 1..19: note = RNA
  misc_feature 20..21: note = DNA
  source 1..21: mol_type = other DNA
gcatttggctgcaaagaaatt

SEQ ID NO: 475 - DNA, 21 bp, synthetic construct
  misc_feature 1..19: note = RNA
  misc_feature 20..21: note = DNA
  source 1..21: mol_type = other DNA
catttggctgcaaagaaattt

SEQ ID NO: 476 - DNA, 21 bp, synthetic construct
  misc_feature 1..19: note = RNA
  misc_feature 20..21: note = DNA
  source 1..21: mol_type = other DNA
gctgcaaagaaatctgctttt

SEQ ID NO: 477 - DNA, 21 bp, synthetic construct
  misc_feature 1..19: note = RNA
  misc_feature 20..21: note = DNA
  source 1..21: mol_type = other DNA
caaagaaatctgcttggaatt

SEQ ID NO: 478 - DNA, 21 bp, synthetic construct
  misc_feature 1..19: note = RNA
  misc_feature 20..21: note = DNA
  source 1..21: mol_type = other DNA
agaaatctgcttggaagaatt

SEQ ID NO: 479 - DNA, 21 bp, synthetic construct
  misc_feature 1..19: note = RNA
  misc_feature 20..21: note = DNA
  source 1..21: mol_type = other DNA
tgcttggaagaaggggttatt

SEQ ID NO: 480 - DNA, 21 bp, synthetic construct
  misc_feature 1..19: note = RNA
  misc_feature 20..21: note = DNA
  source 1..21: mol_type = other DNA
ctgtttggccgggcagaaatt

SEQ ID NO: 481 - DNA, 21 bp, synthetic construct
  misc_feature 1..19: note = RNA
  misc_feature 20..21: note = DNA
  source 1..21: mol_type = other DNA
gaaactccgctgagcagaatt

SEQ ID NO: 482 - DNA, 21 bp, synthetic construct
  misc_feature 1..19: note = RNA
  misc_feature 20..21: note = DNA
  source 1..21: mol_type = other DNA
actccgctgagcagaactttt

SEQ ID NO: 483 - DNA, 21 bp, synthetic construct
  misc_feature 1..19: note = RNA
  misc_feature 20..21: note = DNA
  source 1..21: mol_type = other DNA
gcagaacttgccgccagaatt

SEQ ID NO: 484 - DNA, 21 bp, synthetic construct
  misc_feature 1..19: note = RNA
  misc_feature 20..21: note = DNA
  source 1..21: mol_type = other DNA
cagaacttgccgccagaattt

SEQ ID NO: 485 - DNA, 21 bp, synthetic construct
  misc_feature 1..19: note = RNA
  misc_feature 20..21: note = DNA
  source 1..21: mol_type = other DNA
ggctctgttcctgttcttctt

SEQ ID NO: 486 - DNA, 21 bp, synthetic construct
  misc_feature 1..19: note = RNA
  misc_feature 20..21: note = DNA
  source 1..21: mol_type = other DNA
acctatttataacactttttt

SEQ ID NO: 487 - DNA, 21 bp, synthetic construct
  misc_feature 1..19: note = RNA
  misc_feature 20..21: note = DNA
  source 1..21: mol_type = other DNA
acaataaataaatctcaaatt

SEQ ID NO: 488 - DNA, 21 bp, synthetic construct
  misc_feature 1..19: note = RNA
  misc_feature 20..21: note = DNA
  source 1..21: mol_type = other DNA
cctcgtgttgaatcttaaatt

SEQ ID NO: 489 - DNA, 21 bp, synthetic construct
  misc_feature 1..19: note = RNA
  misc_feature 20..21: note = DNA
  source 1..21: mol_type = other DNA
ataccaactgtgtggactatt

SEQ ID NO: 490 - DNA, 21 bp, synthetic construct
  misc_feature 1..19: note = RNA
  misc_feature 20..21: note = DNA
  source 1..21: mol_type = other DNA
aaacctatttataacactttt

SEQ ID NO: 491 - DNA, 21 bp, synthetic construct
  misc_feature 1..19: note = RNA
  misc_feature 20..21: note = DNA
  source 1..21: mol_type = other DNA
aaaagtgttataaataggttt

SEQ ID NO: 492 - DNA, 21 bp, synthetic construct
  misc_feature 1..19: note = RNA
  misc_feature 20..21: note = DNA
  source 1..21: mol_type = other DNA
aaacagtggtggacatttctt

SEQ ID NO: 493 - DNA, 21 bp, synthetic construct
  misc_feature 1..19: note = RNA
  misc_feature 20..21: note = DNA
  source 1..21: mol_type = other DNA
aaaccgtaggagtaatccgtt

SEQ ID NO: 494 - DNA, 21 bp, synthetic construct
  misc_feature 1..19: note = RNA
  misc_feature 20..21: note = DNA
  source 1..21: mol_type = other DNA
aaagcaaacaatactatgttt

SEQ ID NO: 495 - DNA, 21 bp, synthetic construct
  misc_feature 1..19: note = RNA
  misc_feature 20..21: note = DNA
  source 1..21: mol_type = other DNA
aaagttccttagctacttctt

SEQ ID NO: 496 - DNA, 21 bp, synthetic construct
  misc_feature 1..19: note = RNA
  misc_feature 20..21: note = DNA
  source 1..21: mol_type = other DNA
aaatgcaagggatgttaagtt

SEQ ID NO: 497 - DNA, 21 bp, synthetic construct
  misc_feature 1..19: note = RNA
  misc_feature 20..21: note = DNA
  source 1..21: mol_type = other DNA
aacacgaggctgatggtcatt

SEQ ID NO: 498 - DNA, 21 bp, synthetic construct
  misc_feature 1..19: note = RNA
  misc_feature 20..21: note = DNA
  source 1..21: mol_type = other DNA
aacagagacagaatgctgatt

SEQ ID NO: 499 - DNA, 21 bp, synthetic construct
  misc_feature 1..19: note = RNA
  misc_feature 20..21: note = DNA
  source 1..21: mol_type = other DNA
aacagcagcaccagcaccgtt

SEQ ID NO: 500 - DNA, 21 bp, synthetic construct
  misc_feature 1..19: note = RNA
  misc_feature 20..21: note = DNA
  source 1..21: mol_type = other DNA
aacagtggtggacatttcctt

SEQ ID NO: 501 - DNA, 21 bp, synthetic construct
  misc_feature 1..19: note = RNA
  misc_feature 20..21: note = DNA
  source 1..21: mol_type = other DNA
aagaatttggaagattccatt

SEQ ID NO: 502 - DNA, 21 bp, synthetic construct
  misc_feature 1..19: note = RNA
  misc_feature 20..21: note = DNA
  source 1..21: mol_type = other DNA
aagaggtgctacagttctgtt

SEQ ID NO: 503 - DNA, 21 bp, synthetic construct
  misc_feature 1..19: note = RNA
  misc_feature 20..21: note = DNA
  source 1..21: mol_type = other DNA
aagagttggcagaagaacatt

SEQ ID NO: 504 - DNA, 21 bp, synthetic construct
  misc_feature 1..19: note = RNA
  misc_feature 20..21: note = DNA
  source 1..21: mol_type = other DNA
aagatcacatagatgacactt

SEQ ID NO: 505 - DNA, 21 bp, synthetic construct
  misc_feature 1..19: note = RNA
  misc_feature 20..21: note = DNA
  source 1..21: mol_type = other DNA
aagatgatcgacaggatcatt

SEQ ID NO: 506 - DNA, 21 bp, synthetic construct
  misc_feature 1..19: note = RNA
  misc_feature 20..21: note = DNA
  source 1..21: mol_type = other DNA
aagattccagtgatgtaaatt

SEQ ID NO: 507 - DNA, 21 bp, synthetic construct
  misc_feature 1..19: note = RNA
  misc_feature 20..21: note = DNA
  source 1..21: mol_type = other DNA
aagcagatttctttgcagctt

SEQ ID NO: 508 - DNA, 21 bp, synthetic construct
  misc_feature 1..19: note = RNA
  misc_feature 20..21: note = DNA
  source 1..21: mol_type = other DNA
aaggccacccaggccaggatt

SEQ ID NO: 509 - DNA, 21 bp, synthetic construct
  misc_feature 1..19: note = RNA
  misc_feature 20..21: note = DNA
  source 1..21: mol_type = other DNA
aagggatgttaaggcaagatt

SEQ ID NO: 510 - DNA, 21 bp, synthetic construct
  misc_feature 1..19: note = RNA
  misc_feature 20..21: note = DNA
  source 1..21: mol_type = other DNA
aagtgttataaataggttttt

SEQ ID NO: 511 - DNA, 21 bp, synthetic construct
  misc_feature 1..19: note = RNA
  misc_feature 20..21: note = DNA
  source 1..21: mol_type = other DNA
aagttctgctcagcggagttt

SEQ ID NO: 512 - DNA, 21 bp, synthetic construct
  misc_feature 1..19: note = RNA
  misc_feature 20..21: note = DNA
  source 1..21: mol_type = other DNA
aatcagaaaatgcaaagcatt

SEQ ID NO: 513 - DNA, 21 bp, synthetic construct
  misc_feature 1..19: note = RNA
  misc_feature 20..21: note = DNA
  source 1..21: mol_type = other DNA
aatccgagttgagatgccatt

SEQ ID NO: 514 - DNA, 21 bp, synthetic construct
  misc_feature 1..19: note = RNA
  misc_feature 20..21: note = DNA
  source 1..21: mol_type = other DNA
aatgctgaagatgatcgactt

SEQ ID NO: 515 - DNA, 21 bp, synthetic construct
  misc_feature 1..19: note = RNA
  misc_feature 20..21: note = DNA
  source 1..21: mol_type = other DNA
aatttggaagattccagtgtt

SEQ ID NO: 516 - DNA, 21 bp, synthetic construct
  misc_feature 1..19: note = RNA
  misc_feature 20..21: note = DNA
  source 1..21: mol_type = other DNA
acagaccagcaagaatttgtt

SEQ ID NO: 517 - DNA, 21 bp, synthetic construct
  misc_feature 1..19: note = RNA
  misc_feature 20..21: note = DNA
  source 1..21: mol_type = other DNA
acagactaagcctgcagcttt

SEQ ID NO: 518 - DNA, 21 bp, synthetic construct
  misc_feature 1..19: note = RNA
  misc_feature 20..21: note = DNA
  source 1..21: mol_type = other DNA
acagactgcagccattcgttt

SEQ ID NO: 519 - DNA, 21 bp, synthetic construct
  misc_feature 1..19: note = RNA
  misc_feature 20..21: note = DNA
  source 1..21: mol_type = other DNA
acaggatcatggtggcctgtt

SEQ ID NO: 520 - DNA, 21 bp, synthetic construct
  misc_feature 1..19: note = RNA
  misc_feature 20..21: note = DNA
  source 1..21: mol_type = other DNA
acagggcagtcagagaccctt

SEQ ID NO: 521 - DNA, 21 bp, synthetic construct
  misc_feature 1..19: note = RNA
  misc_feature 20..21: note = DNA
  source 1..21: mol_type = other DNA
acagttctgccagagatcatt

SEQ ID NO: 522 - DNA, 21 bp, synthetic construct
  misc_feature 1..19: note = RNA
  misc_feature 20..21: note = DNA
  source 1..21: mol_type = other DNA
acatttcctgaggaagaggtt

SEQ ID NO: 523 - DNA, 21 bp, synthetic construct
  misc_feature 1..19: note = RNA
  misc_feature 20..21: note = DNA
  source 1..21: mol_type = other DNA
acgatgatactcagcaacatt

SEQ ID NO: 524 - DNA, 21 bp, synthetic construct
  misc_feature 1..19: note = RNA
  misc_feature 20..21: note = DNA
  source 1..21: mol_type = other DNA
acgtggaggacgatgatactt

SEQ ID NO: 525 - DNA, 21 bp, synthetic construct
  misc_feature 1..19: note = RNA
  misc_feature 20..21: note = DNA
  source 1..21: mol_type = other DNA
actaagcctgcagcttccatt

SEQ ID NO: 526 - DNA, 21 bp, synthetic construct
  misc_feature 1..19: note = RNA
  misc_feature 20..21: note = DNA
  source 1..21: mol_type = other DNA
actcagcaacaggaggagctt

SEQ ID NO: 527 - DNA, 21 bp, synthetic construct
  misc_feature 1..19: note = RNA
  misc_feature 20..21: note = DNA
  source 1..21: mol_type = other DNA
actcatcacgcacagaccatt

SEQ ID NO: 528 - DNA, 21 bp, synthetic construct
  misc_feature 1..19: note = RNA
  misc_feature 20..21: note = DNA
  source 1..21: mol_type = other DNA
actccgggtgcctcaccgttt

SEQ ID NO: 529 - DNA, 21 bp, synthetic construct
  misc_feature 1..19: note = RNA
  misc_feature 20..21: note = DNA
  source 1..21: mol_type = other DNA
agaagaacaggaacagagatt

SEQ ID NO: 530 - DNA, 21 bp, synthetic construct
  misc_feature 1..19: note = RNA
  misc_feature 20..21: note = DNA
  source 1..21: mol_type = other DNA
agaatttggaagattccagtt

SEQ ID NO: 531 - DNA, 21 bp, synthetic construct
  misc_feature 1..19: note = RNA
  misc_feature 20..21: note = DNA
  source 1..21: mol_type = other DNA
agacgaacagcagcaccagtt

SEQ ID NO: 532 - DNA, 21 bp, synthetic construct
  misc_feature 1..19: note = RNA
  misc_feature 20..21: note = DNA
  source 1..21: mol_type = other DNA
agagatcagttgcgtgtcctt

SEQ ID NO: 533 - DNA, 21 bp, synthetic construct
  misc_feature 1..19: note = RNA
  misc_feature 20..21: note = DNA
  source 1..21: mol_type = other DNA
agcaagaatttggaagatttt

SEQ ID NO: 534 - DNA, 21 bp, synthetic construct
  misc_feature 1..19: note = RNA
  misc_feature 20..21: note = DNA
  source 1..21: mol_type = other DNA
agcagcaccagcaccgcgatt

SEQ ID NO: 535 - DNA, 21 bp, synthetic construct
  misc_feature 1..19: note = RNA
  misc_feature 20..21: note = DNA
  source 1..21: mol_type = other DNA
aggatgtaggcgaaaccgttt

SEQ ID NO: 536 - DNA, 21 bp, synthetic construct
  misc_feature 1..19: note = RNA
  misc_feature 20..21: note = DNA
  source 1..21: mol_type = other DNA
agtgatgtaaaacctgccctt

SEQ ID NO: 537 - DNA, 21 bp, synthetic construct
  misc_feature 1..19: note = RNA
  misc_feature 20..21: note = DNA
  source 1..21: mol_type = other DNA
agttctgccagagatcagttt

SEQ ID NO: 538 - DNA, 21 bp, synthetic construct
  misc_feature 1..19: note = RNA
  misc_feature 20..21: note = DNA
  source 1..21: mol_type = other DNA
agttggcagaagaacaggatt

SEQ ID NO: 539 - DNA, 21 bp, synthetic construct
  misc_feature 1..19: note = RNA
  misc_feature 20..21: note = DNA
  source 1..21: mol_type = other DNA
ataaataggttttataaactt

SEQ ID NO: 540 - DNA, 21 bp, synthetic construct
  misc_feature 1..19: note = RNA
  misc_feature 20..21: note = DNA
  source 1..21: mol_type = other DNA
atacatctacagttggtggtt

SEQ ID NO: 541 - DNA, 21 bp, synthetic construct
  misc_feature 1..19: note = RNA
  misc_feature 20..21: note = DNA
  source 1..21: mol_type = other DNA
atacatcttcaatcaacagtt

SEQ ID NO: 542 - DNA, 21 bp, synthetic construct
  misc_feature 1..19: note = RNA
  misc_feature 20..21: note = DNA
  source 1..21: mol_type = other DNA
atactcagcaacaggaggatt

SEQ ID NO: 543 - DNA, 21 bp, synthetic construct
  misc_feature 1..19: note = RNA
  misc_feature 20..21: note = DNA
  source 1..21: mol_type = other DNA
atagatgacaccgctgagatt

SEQ ID NO: 544 - DNA, 21 bp, synthetic construct
  misc_feature 1..19: note = RNA
  misc_feature 20..21: note = DNA
  source 1..21: mol_type = other DNA
ataggttttataaaccggatt

SEQ ID NO: 545 - DNA, 21 bp, synthetic construct
  misc_feature 1..19: note = RNA
  misc_feature 20..21: note = DNA
  source 1..21: mol_type = other DNA
atcacatagatgacaccgctt

SEQ ID NO: 546 - DNA, 21 bp, synthetic construct
  misc_feature 1..19: note = RNA
  misc_feature 20..21: note = DNA
  source 1..21: mol_type = other DNA
atcacgcacagaccagcaatt

SEQ ID NO: 547 - DNA, 21 bp, synthetic construct
  misc_feature 1..19: note = RNA
  misc_feature 20..21: note = DNA
  source 1..21: mol_type = other DNA
atcagttgcgtgtccattgtt

SEQ ID NO: 548 - DNA, 21 bp, synthetic construct
  misc_feature 1..19: note = RNA
  misc_feature 20..21: note = DNA
  source 1..21: mol_type = other DNA
atcatggtggcctggacagtt

SEQ ID NO: 549 - DNA, 21 bp, synthetic construct
  misc_feature 1..19: note = RNA
  misc_feature 20..21: note = DNA
  source 1..21: mol_type = other DNA
atccattggctgacgatcgtt

SEQ ID NO: 550 - DNA, 21 bp, synthetic construct
  misc_feature 1..19: note = RNA
  misc_feature 20..21: note = DNA
  source 1..21: mol_type = other DNA
atcgacaggatcatggtggtt

SEQ ID NO: 551 - DNA, 21 bp, synthetic construct
  misc_feature 1..19: note = RNA
  misc_feature 20..21: note = DNA
  source 1..21: mol_type = other DNA
atcgtggagacgaacagcatt

SEQ ID NO: 552 - DNA, 21 bp, synthetic construct
  misc_feature 1..19: note = RNA
  misc_feature 20..21: note = DNA
  source 1..21: mol_type = other DNA
atcttagtccacacagttgtt

SEQ ID NO: 553 - DNA, 21 bp, synthetic construct
  misc_feature 1..19: note = RNA
  misc_feature 20..21: note = DNA
  source 1..21: mol_type = other DNA
atgacaccgctgagaagggtt

SEQ ID NO: 554 - DNA, 21 bp, synthetic construct
  misc_feature 1..19: note = RNA
  misc_feature 20..21: note = DNA
  source 1..21: mol_type = other DNA
atgatactcagcaacaggatt

SEQ ID NO: 555 - DNA, 21 bp, synthetic construct
  misc_feature 1..19: note = RNA
  misc_feature 20..21: note = DNA
  source 1..21: mol_type = other DNA
atgatcgacaggatcatggtt

SEQ ID NO: 556 - DNA, 21 bp, synthetic construct
  misc_feature 1..19: note = RNA
  misc_feature 20..21: note = DNA
  source 1..21: mol_type = other DNA
atgatgagaaacagtggtgtt

SEQ ID NO: 557 - DNA, 21 bp, synthetic construct
  misc_feature 1..19: note = RNA
  misc_feature 20..21: note = DNA
  source 1..21: mol_type = other DNA
atgcaagggatgttaaggctt

SEQ ID NO: 558 - DNA, 21 bp, synthetic construct
  misc_feature 1..19: note = RNA
  misc_feature 20..21: note = DNA
  source 1..21: mol_type = other DNA
atgcatcttagtccacacatt

SEQ ID NO: 559 - DNA, 21 bp, synthetic construct
  misc_feature 1..19: note = RNA
  misc_feature 20..21: note = DNA
  source 1..21: mol_type = other DNA
atgccactccgggtgcctctt

SEQ ID NO: 560 - DNA, 21 bp, synthetic construct
  misc_feature 1..19: note = RNA
  misc_feature 20..21: note = DNA
  source 1..21: mol_type = other DNA
atgctgaagatgatcgacatt

SEQ ID NO: 561 - DNA, 21 bp, synthetic construct
  misc_feature 1..19: note = RNA
  misc_feature 20..21: note = DNA
  source 1..21: mol_type = other DNA
atggccgcagcactcatcatt

SEQ ID NO: 562 - DNA, 21 bp, synthetic construct
  misc_feature 1..19: note = RNA
  misc_feature 20..21: note = DNA
  source 1..21: mol_type = other DNA
atggtggcctggacagacttt

SEQ ID NO: 563 - DNA, 21 bp, synthetic construct
  misc_feature 1..19: note = RNA
  misc_feature 20..21: note = DNA
  source 1..21: mol_type = other DNA
atgtaggcgaaaccgtaggtt

SEQ ID NO: 564 - DNA, 21 bp, synthetic construct
  misc_feature 1..19: note = RNA
  misc_feature 20..21: note = DNA
  source 1..21: mol_type = other DNA
atgttaaggcaagaccctctt

SEQ ID NO: 565 - DNA, 21 bp, synthetic construct
  misc_feature 1..19: note = RNA
  misc_feature 20..21: note = DNA
  source 1..21: mol_type = other DNA
atgttccctggtggtgctctt

SEQ ID NO: 566 - DNA, 21 bp, synthetic construct
  misc_feature 1..19: note = RNA
  misc_feature 20..21: note = DNA
  source 1..21: mol_type = other DNA
attatactgttaggatgtatt

SEQ ID NO: 567 - DNA, 21 bp, synthetic construct
  misc_feature 1..19: note = RNA
  misc_feature 20..21: note = DNA
  source 1..21: mol_type = other DNA
attccagtgatgtaaaacctt

SEQ ID NO: 568 - DNA, 21 bp, synthetic construct
  misc_feature 1..19: note = RNA
  misc_feature 20..21: note = DNA
  source 1..21: mol_type = other DNA
attcgcgtttccgcaagattt

SEQ ID NO: 569 - DNA, 21 bp, synthetic construct
  misc_feature 1..19: note = RNA
  misc_feature 20..21: note = DNA
  source 1..21: mol_type = other DNA
attcgtttggtgatgatgatt

SEQ ID NO: 570 - DNA, 21 bp, synthetic construct
  misc_feature 1..19: note = RNA
  misc_feature 20..21: note = DNA
  source 1..21: mol_type = other DNA
attctggcggcaagttctgtt

SEQ ID NO: 571 - DNA, 21 bp, synthetic construct
  misc_feature 1..19: note = RNA
  misc_feature 20..21: note = DNA
  source 1..21: mol_type = other DNA
attgcccacgatccattggtt

SEQ ID NO: 572 - DNA, 21 bp, synthetic construct
  misc_feature 1..19: note = RNA
  misc_feature 20..21: note = DNA
  source 1..21: mol_type = other DNA
attggctgacgatcgtggatt

SEQ ID NO: 573 - DNA, 21 bp, synthetic construct
  misc_feature 1..19: note = RNA
  misc_feature 20..21: note = DNA
  source 1..21: mol_type = other DNA
atttcctgaggaagaggtgtt

SEQ ID NO: 574 - DNA, 21 bp, synthetic construct
  misc_feature 1..19: note = RNA
  misc_feature 20..21: note = DNA
  source 1..21: mol_type = other DNA
atttctttgcagccaaatgtt

SEQ ID NO: 575 - DNA, 21 bp, synthetic construct
  misc_feature 1..19: note = RNA
  misc_feature 20..21: note = DNA
  source 1..21: mol_type = other DNA
atttggaagattccagtgatt

SEQ ID NO: 576 - DNA, 21 bp, synthetic construct
  misc_feature 1..19: note = RNA
  misc_feature 20..21: note = DNA
  source 1..21: mol_type = other DNA
cagaagaacaggaacagagtt

SEQ ID NO: 577 - DNA, 21 bp, synthetic construct
  misc_feature 1..19: note = RNA
  misc_feature 20..21: note = DNA
  source 1..21: mol_type = other DNA
cagttctgccagagatcagtt

SEQ ID NO: 578 - DNA, 21 bp, synthetic construct
  misc_feature 1..19: note = RNA
  misc_feature 20..21: note = DNA
  source 1..21: mol_type = other DNA
cattcgtttggtgatgatgtt

SEQ ID NO: 579 - DNA, 21 bp, synthetic construct
  misc_feature 1..19: note = RNA
  misc_feature 20..21: note = DNA
  source 1..21: mol_type = other DNA
cgatgatactcagcaacagtt

SEQ ID NO: 580 - DNA, 21 bp, synthetic construct
  misc_feature 1..19: note = RNA
  misc_feature 20..21: note = DNA
  source 1..21: mol_type = other DNA
gaagaacaggaacagagcctt

SEQ ID NO: 581 - DNA, 21 bp, synthetic construct
  misc_feature 1..19: note = RNA
  misc_feature 20..21: note = DNA
  source 1..21: mol_type = other DNA
gacgatgatactcagcaactt

SEQ ID NO: 582 - DNA, 21 bp, synthetic construct
  misc_feature 1..19: note = RNA
  misc_feature 20..21: note = DNA
  source 1..21: mol_type = other DNA
gatactcagcaacaggaggtt

SEQ ID NO: 583 - DNA, 21 bp, synthetic construct
  misc_feature 1..19: note = RNA
  misc_feature 20..21: note = DNA
  source 1..21: mol_type = other DNA
gatgatactcagcaacaggtt

SEQ ID NO: 584 - DNA, 21 bp, synthetic construct
  misc_feature 1..19: note = RNA
  misc_feature 20..21: note = DNA
  source 1..21: mol_type = other DNA
gcagaagaacaggaacagatt

SEQ ID NO: 585 - DNA, 21 bp, synthetic construct
  misc_feature 1..19: note = RNA
  misc_feature 20..21: note = DNA
  source 1..21: mol_type = other DNA
ggacgatgatactcagcaatt

SEQ ID NO: 586 - DNA, 21 bp, synthetic construct
  misc_feature 1..19: note = RNA
  misc_feature 20..21: note = DNA
  source 1..21: mol_type = other DNA
taaagcttcacacagaggttt

SEQ ID NO: 587 - DNA, 21 bp, synthetic construct
  misc_feature 1..19: note = RNA
  misc_feature 20..21: note = DNA
  source 1..21: mol_type = other DNA
taaccccttcttccaagcatt

SEQ ID NO: 588 - DNA, 21 bp, synthetic construct
  misc_feature 1..19: note = RNA
  misc_feature 20..21: note = DNA
  source 1..21: mol_type = other DNA
taagcctgcagcttccaactt

SEQ ID NO: 589 - DNA, 21 bp, synthetic construct
  misc_feature 1..19: note = RNA
  misc_feature 20..21: note = DNA
  source 1..21: mol_type = other DNA
taatccgagttgagatgcctt

SEQ ID NO: 590 - DNA, 21 bp, synthetic construct
  misc_feature 1..19: note = RNA
  misc_feature 20..21: note = DNA
  source 1..21: mol_type = other DNA
tacagttctgccagagatctt

SEQ ID NO: 591 - DNA, 21 bp, synthetic construct
  misc_feature 1..19: note = RNA
  misc_feature 20..21: note = DNA
  source 1..21: mol_type = other DNA
tacatcttcaatcaacagctt

SEQ ID NO: 592 - DNA, 21 bp, synthetic construct
  misc_feature 1..19: note = RNA
  misc_feature 20..21: note = DNA
  source 1..21: mol_type = other DNA
tactcagcaacaggaggagtt

SEQ ID NO: 593 - DNA, 21 bp, synthetic construct
  misc_feature 1..19: note = RNA
  misc_feature 20..21: note = DNA
  source 1..21: mol_type = other DNA
tagatgacaccgctgagaatt

SEQ ID NO: 594 - DNA, 21 bp, synthetic construct
  misc_feature 1..19: note = RNA
  misc_feature 20..21: note = DNA
  source 1..21: mol_type = other DNA
tagatggccgcagcactcatt

SEQ ID NO: 595 - DNA, 21 bp, synthetic construct
  misc_feature 1..19: note = RNA
  misc_feature 20..21: note = DNA
  source 1..21: mol_type = other DNA
taggagtaatccgagttgatt

SEQ ID NO: 596 - DNA, 21 bp, synthetic construct
  misc_feature 1..19: note = RNA
  misc_feature 20..21: note = DNA
  source 1..21: mol_type = other DNA
taggcgaaaccgtaggagttt

SEQ ID NO: 597 - DNA, 21 bp, synthetic construct
  misc_feature 1..19: note = RNA
  misc_feature 20..21: note = DNA
  source 1..21: mol_type = other DNA
tagtccacacagttggtattt

SEQ ID NO: 598 - DNA, 21 bp, synthetic construct
  misc_feature 1..19: note = RNA
  misc_feature 20..21: note = DNA
  source 1..21: mol_type = other DNA
tatgtacgctcagagcctctt

SEQ ID NO: 599 - DNA, 21 bp, synthetic construct
  misc_feature 1..19: note = RNA
  misc_feature 20..21: note = DNA
  source 1..21: mol_type = other DNA
tcaccgtgtagatggccgctt

SEQ ID NO: 600 - DNA, 21 bp, synthetic construct
  misc_feature 1..19: note = RNA
  misc_feature 20..21: note = DNA
  source 1..21: mol_type = other DNA
tcatcacgcacagaccagctt

SEQ ID NO: 601 - DNA, 21 bp, synthetic construct
  misc_feature 1..19: note = RNA
  misc_feature 20..21: note = DNA
  source 1..21: mol_type = other DNA
tcatggtggcctggacagatt

SEQ ID NO: 602 - DNA, 21 bp, synthetic construct
  misc_feature 1..19: note = RNA
  misc_feature 20..21: note = DNA
  source 1..21: mol_type = other DNA
tctggagtcttagcatcagtt

SEQ ID NO: 603 - DNA, 21 bp, synthetic construct
  misc_feature 1..19: note = RNA
  misc_feature 20..21: note = DNA
  source 1..21: mol_type = other DNA
tgacgatcgtggagacgaatt

SEQ ID NO: 604 - DNA, 21 bp, synthetic construct
  misc_feature 1..19: note = RNA
  misc_feature 20..21: note = DNA
  source 1..21: mol_type = other DNA
tgagggtgaagagttggcatt

SEQ ID NO: 605 - DNA, 21 bp, synthetic construct
  misc_feature 1..19: note = RNA
  misc_feature 20..21: note = DNA
  source 1..21: mol_type = other DNA
tgatactcagcaacaggagtt

SEQ ID NO: 606 - DNA, 21 bp, synthetic construct
  misc_feature 1..19: note = RNA
  misc_feature 20..21: note = DNA
  source 1..21: mol_type = other DNA
tgcagcttccaaccaggcttt

SEQ ID NO: 607 - DNA, 21 bp, synthetic construct
  misc_feature 1..19: note = RNA
  misc_feature 20..21: note = DNA
  source 1..21: mol_type = other DNA
tggagacgaacagcagcactt

SEQ ID NO: 608 - DNA, 21 bp, synthetic construct
  misc_feature 1..19: note = RNA
  misc_feature 20..21: note = DNA
  source 1..21: mol_type = other DNA
tggaggacgatgatactcatt

SEQ ID NO: 609 - DNA, 21 bp, synthetic construct
  misc_feature 1..19: note = RNA
  misc_feature 20..21: note = DNA
  source 1..21: mol_type = other DNA
ttagctacttctttaaggctt

SEQ ID NO: 610 - DNA, 21 bp, synthetic construct
  misc_feature 1..19: note = RNA
  misc_feature 20..21: note = DNA
  source 1..21: mol_type = other DNA
ttaggatgtaaagttcctttt

SEQ ID NO: 611 - DNA, 21 bp, synthetic construct
  misc_feature 1..19: note = RNA
  misc_feature 20..21: note = DNA
  source 1..21: mol_type = other DNA
ttagtccacacagttggtatt

SEQ ID NO: 612 - DNA, 21 bp, synthetic construct
  misc_feature 1..19: note = RNA
  misc_feature 20..21: note = DNA
  source 1..21: mol_type = other DNA
ttatactgttaggatgtaatt

SEQ ID NO: 613 - DNA, 21 bp, synthetic construct
  misc_feature 1..19: note = RNA
  misc_feature 20..21: note = DNA
  source 1..21: mol_type = other DNA
ttatcttatttctgggtaatt

SEQ ID NO: 614 - DNA, 21 bp, synthetic construct
  misc_feature 1..19: note = RNA
  misc_feature 20..21: note = DNA
  source 1..21: mol_type = other DNA
ttcagatgaaagggaagggtt

SEQ ID NO: 615 - DNA, 21 bp, synthetic construct
  misc_feature 1..19: note = RNA
  misc_feature 20..21: note = DNA
  source 1..21: mol_type = other DNA
ttccaagcagatttctttgtt

SEQ ID NO: 616 - DNA, 21 bp, synthetic construct
  misc_feature 1..19: note = RNA
  misc_feature 20..21: note = DNA
  source 1..21: mol_type = other DNA
ttccctggtggtgctcccttt

SEQ ID NO: 617 - DNA, 21 bp, synthetic construct
  misc_feature 1..19: note = RNA
  misc_feature 20..21: note = DNA
  source 1..21: mol_type = other DNA
ttctgccagagatcagttgtt

SEQ ID NO: 618 - DNA, 21 bp, synthetic construct
  misc_feature 1..19: note = RNA
  misc_feature 20..21: note = DNA
  source 1..21: mol_type = other DNA
ttctgctcagcggagtttctt

SEQ ID NO: 619 - DNA, 21 bp, synthetic construct
  misc_feature 1..19: note = RNA
  misc_feature 20..21: note = DNA
  source 1..21: mol_type = other DNA
ttctggcggcaagttctgctt

SEQ ID NO: 620 - DNA, 21 bp, synthetic construct
  misc_feature 1..19: note = RNA
  misc_feature 20..21: note = DNA
  source 1..21: mol_type = other DNA
ttcttccaagcagatttcttt

SEQ ID NO: 621 - DNA, 21 bp, synthetic construct
  misc_feature 1..19: note = RNA
  misc_feature 20..21: note = DNA
  source 1..21: mol_type = other DNA
ttctttgcagccaaatgcatt

SEQ ID NO: 622 - DNA, 21 bp, synthetic construct
  misc_feature 1..19: note = RNA
  misc_feature 20..21: note = DNA
  source 1..21: mol_type = other DNA
ttgagatgccactccgggttt

SEQ ID NO: 623 - DNA, 21 bp, synthetic construct
  misc_feature 1..19: note = RNA
  misc_feature 20..21: note = DNA
  source 1..21: mol_type = other DNA
ttgagtttgggattttgggtt

SEQ ID NO: 624 - DNA, 21 bp, synthetic construct
  misc_feature 1..19: note = RNA
  misc_feature 20..21: note = DNA
  source 1..21: mol_type = other DNA
ttgcagccaaatgcaagggtt

SEQ ID NO: 625 - DNA, 21 bp, synthetic construct
  misc_feature 1..19: note = RNA
  misc_feature 20..21: note = DNA
  source 1..21: mol_type = other DNA
ttgcccacgatccattggctt

SEQ ID NO: 626 - DNA, 21 bp, synthetic construct
  misc_feature 1..19: note = RNA
  misc_feature 20..21: note = DNA
  source 1..21: mol_type = other DNA
ttgcgtgtccattgcccactt

SEQ ID NO: 627 - DNA, 21 bp, synthetic construct
  misc_feature 1..19: note = RNA
  misc_feature 20..21: note = DNA
  source 1..21: mol_type = other DNA
ttggcagaagaacaggaactt

SEQ ID NO: 628 - DNA, 21 bp, synthetic construct
  misc_feature 1..19: note = RNA
  misc_feature 20..21: note = DNA
  source 1..21: mol_type = other DNA
ttggtgagggtgaagagtttt

SEQ ID NO: 629 - DNA, 21 bp, synthetic construct
  misc_feature 1..19: note = RNA
  misc_feature 20..21: note = DNA
  source 1..21: mol_type = other DNA
ttggtgatgatgagaaacatt

SEQ ID NO: 630 - DNA, 21 bp, synthetic construct
  misc_feature 1..19: note = RNA
  misc_feature 20..21: note = DNA
  source 1..21: mol_type = other DNA
tttaagattcaacacgaggtt

SEQ ID NO: 631 - DNA, 21 bp, synthetic construct
  misc_feature 1..19: note = RNA
  misc_feature 20..21: note = DNA
  source 1..21: mol_type = other DNA
tttccgcaagatcacatagtt

SEQ ID NO: 632 - DNA, 21 bp, synthetic construct
  misc_feature 1..19: note = RNA
  misc_feature 20..21: note = DNA
  source 1..21: mol_type = other DNA
tttcctgaggaagaggtgctt

SEQ ID NO: 633 - DNA, 21 bp, synthetic construct
  misc_feature 1..19: note = RNA
  misc_feature 20..21: note = DNA
  source 1..21: mol_type = other DNA
tttctgcccggccaaacagtt

SEQ ID NO: 634 - DNA, 21 bp, synthetic construct
  misc_feature 1..19: note = RNA
  misc_feature 20..21: note = DNA
  source 1..21: mol_type = other DNA
tttctgttggatgcactggtt

SEQ ID NO: 635 - DNA, 21 bp, synthetic construct
  misc_feature 1..19: note = RNA
  misc_feature 20..21: note = DNA
  source 1..21: mol_type = other DNA
tttctgttttcccttcctctt

SEQ ID NO: 636 - DNA, 21 bp, synthetic construct
  misc_feature 1..19: note = RNA
  misc_feature 20..21: note = DNA
  source 1..21: mol_type = other DNA
tttctttgcagccaaatgctt

SEQ ID NO: 637 - DNA, 21 bp, synthetic construct
  misc_feature 1..19: note = RNA
  misc_feature 20..21: note = DNA
  source 1..21: mol_type = other DNA
tttgagatttatttattgttt

SEQ ID NO: 638 - DNA, 21 bp, synthetic construct
  misc_feature 1..19: note = RNA
  misc_feature 20..21: note = DNA
  source 1..21: mol_type = other DNA
tttgcagccaaatgcaaggtt

SEQ ID NO: 639 - DNA, 21 bp, synthetic construct
  misc_feature 1..19: note = RNA
  misc_feature 20..21: note = DNA
  source 1..21: mol_type = other DNA
tttggaagattccagtgattt

SEQ ID NO: 640 - DNA, 21 bp, synthetic construct
  misc_feature 1..19: note = RNA
  misc_feature 20..21: note = DNA
  source 1..21: mol_type = other DNA
tttggtgatgatgagaaactt

SEQ ID NO: 641 - DNA, 21 bp, synthetic construct
  misc_feature 1..19: note = RNA
  misc_feature 20..21: note = DNA
  source 1..21: mol_type = other DNA
tttggtttgagtttgggattt

SEQ ID NO: 642 - DNA, 21 bp, synthetic construct
  misc_feature 1..19: note = RNA
  misc_feature 20..21: note = DNA
  source 1..21: mol_type = other DNA
ttatactgttaggatgtaatt

SEQ ID NO: 643 - DNA, 21 bp, synthetic construct
  misc_feature 1..19: note = RNA
  misc_feature 20..21: note = DNA
  source 1..21: mol_type = other DNA
ttatcttatttctgggtaatt

SEQ ID NO: 644 - DNA, 21 bp, synthetic construct
  misc_feature 1..19: note = RNA
  misc_feature 20..21: note = DNA
  source 1..21: mol_type = other DNA
aatcagaaaatgcaaagcatt

SEQ ID NO: 645 - DNA, 21 bp, synthetic construct
  misc_feature 1..19: note = RNA
  misc_feature 20..21: note = DNA
  source 1..21: mol_type = other DNA
atcttagtccacacagttgtt

SEQ ID NO: 646 - RNA, 19 nt, synthetic construct
  source 1..19: mol_type = other RNA
gatctctggcagaactgta

SEQ ID NO: 647 - RNA, 19 nt, synthetic construct
  source 1..19: mol_type = other RNA
ctctgttcctgttcttctg

SEQ ID NO: 648 - RNA, 19 nt, synthetic construct
  source 1..19: mol_type = other RNA
ttgctggtctgtgcgtgat

SEQ ID NO: 649 - RNA, 19 nt, synthetic construct
  source 1..19: mol_type = other RNA
ctctgttcctgttcttctg

SEQ ID NO: 650 - RNA, 19 nt, synthetic construct
  source 1..19: mol_type = other RNA
gtcgatcatcttcagcatt

SEQ ID NO: 651 - RNA, 19 nt, synthetic construct
  source 1..19: mol_type = other RNA
atcactggaatcttccaaa

SEQ ID NO: 652 - RNA, 19 nt, synthetic construct
  source 1..19: mol_type = other RNA
tcctgttgctgagtatcat

SEQ ID NO: 653 - RNA, 19 nt, synthetic construct
  source 1..19: mol_type = other RNA
tcatcatcaccaaacgaat

SEQ ID NO: 654 - RNA, 19 nt, synthetic construct
  source 1..19: mol_type = other RNA
tcagcattctgtctctgtt

SEQ ID NO: 655 - RNA, 19 nt, synthetic construct
  source 1..19: mol_type = other RNA
tctctgttcctgttcttct

SEQ ID NO: 656 - RNA, 19 nt, synthetic construct
  source 1..19: mol_type = other RNA
tcactggaatcttccaaat

SEQ ID NO: 657 - RNA, 19 nt, synthetic construct
  source 1..19: mol_type = other RNA
tctcagcggtgtcatctat

SEQ ID NO: 658 - RNA, 21 nt, synthetic construct
  source 1..21: mol_type = other RNA
cctcctgttgctgagtatcat

SEQ ID NO: 659 - RNA, 19 nt, synthetic construct
  source 1..19: mol_type = other RNA
gctcctcctgttgctgagt

SEQ ID NO: 660 - RNA, 19 nt, synthetic construct
  source 1..19: mol_type = other RNA
tcctcctgttgctgagtat

SEQ ID NO: 661 - RNA, 19 nt, synthetic construct
  source 1..19: mol_type = other RNA
cctcctgttgctgagtatc

SEQ ID NO: 662 - RNA, 19 nt, synthetic construct
  source 1..19: mol_type = other RNA
cctgttgctgagtatcatc

SEQ ID NO: 663 - RNA, 19 nt, synthetic construct
  source 1..19: mol_type = other RNA
ttgctgagtatcatcgtcc

SEQ ID NO: 664 - RNA, 19 nt, synthetic construct
  source 1..19: mol_type = other RNA
caatggacacgcaactgat

SEQ ID NO: 665 - RNA, 19 nt, synthetic construct
  source 1..19: mol_type = other RNA
ccaccatgatcctgtcgat

SEQ ID NO: 666 - RNA, 19 nt, synthetic construct
  source 1..19: mol_type = other RNA
tgtcgatcatcttcagcat

SEQ ID NO: 667 - RNA, 19 nt, synthetic construct
  source 1..19: mol_type = other RNA
tcctgttcttctgccaact

SEQ ID NO: 668 - RNA, 21 nt, synthetic construct
  source 1..21: mol_type = other RNA
cctcctgttgctgagtatcat

SEQ ID NO: 669 - RNA, 19 nt, synthetic construct
  source 1..19: mol_type = other RNA
tttacatcactggaatctt

SEQ ID NO: 670 - RNA, 19 nt, synthetic construct
  source 1..19: mol_type = other RNA
tttacatcactggaatctt

SEQ ID NO: 671 - RNA, 19 nt, synthetic construct
  source 1..19: mol_type = other RNA
aactcttcaccctcaccaa

SEQ ID NO: 672 - RNA, 19 nt, synthetic construct
  source 1..19: mol_type = other RNA
gggcaggttttacatcact

SEQ ID NO: 673 - RNA, 19 nt, synthetic construct
  source 1..19: mol_type = other RNA
ggttttacatcactggaat

SEQ ID NO: 674 - RNA, 19 nt, synthetic construct
  source 1..19: mol_type = other RNA
cactggaatcttccaaatt

SEQ ID NO: 675 - RNA, 19 nt, synthetic construct
  source 1..19: mol_type = other RNA
aatcttccaaattcttgct

SEQ ID NO: 676 - RNA, 19 nt, synthetic construct
  source 1..19: mol_type = other RNA
caaattcttgctggtctgt

SEQ ID NO: 677 - RNA, 19 nt, synthetic construct
  source 1..19: mol_type = other RNA
gctggtctgtgcgtgatga

SEQ ID NO: 678 - RNA, 19 nt, synthetic construct
  source 1..19: mol_type = other RNA
tgatgagtgctgcggccat

SEQ ID NO: 679 - RNA, 19 nt, synthetic construct
  source 1..19: mol_type = other RNA
gaggcacccggagtggcat

SEQ ID NO: 680 - RNA, 19 nt, synthetic construct
  source 1..19: mol_type = other RNA
acccggagtggcatctcaa

SEQ ID NO: 681 - RNA, 19 nt, synthetic construct
  source 1..19: mol_type = other RNA
tcaactcggattactccta

SEQ ID NO: 682 - RNA, 19 nt, synthetic construct
  source 1..19: mol_type = other RNA
cggattactcctacggttt

SEQ ID NO: 683 - RNA, 19 nt, synthetic construct
  source 1..19: mol_type = other RNA
cctacggtttcgcctacat

SEQ ID NO: 684 - RNA, 19 nt, synthetic construct
  source 1..19: mol_type = other RNA
acggtttcgcctacatcct

SEQ ID NO: 685 - RNA, 19 nt, synthetic construct
  source 1..19: mol_type = other RNA
cccttctcagcggtgtcat

SEQ ID NO: 686 - RNA, 19 nt, synthetic construct
  source 1..19: mol_type = other RNA
gcggtgtcatctatgtgat

SEQ ID NO: 687 - RNA, 19 nt, synthetic construct
  source 1..19: mol_type = other RNA
gtgtcatctatgtgatctt

SEQ ID NO: 688 - RNA, 19 nt, synthetic construct
  source 1..19: mol_type = other RNA
ctatgtgatcttgcggaaa

SEQ ID NO: 689 - RNA, 19 nt, synthetic construct
  source 1..19: mol_type = other RNA
gaggaagggaaaacagaaa

SEQ ID NO: 690 - RNA, 19 nt, synthetic construct
  source 1..19: mol_type = other RNA
cccaaaatcccaaactcaa

SEQ ID NO: 691 - RNA, 19 nt, synthetic construct
  source 1..19: mol_type = other RNA
atcccaaactcaaaccaaa

SEQ ID NO: 692 - RNA, 19 nt, synthetic construct
  source 1..19: mol_type = other RNA
gctgttgattgaagatgta

SEQ ID NO: 693 - RNA, 19 nt, synthetic construct
  source 1..19: mol_type = other RNA
ctgttgattgaagatgtat

SEQ ID NO: 694 - RNA, 19 nt, synthetic construct
  source 1..19: mol_type = other RNA
gtttataaaacctatttat

SEQ ID NO: 695 - RNA, 19 nt, synthetic construct
  source 1..19: mol_type = other RNA
acatagtattgtttgcttt

SEQ ID NO: 696 - RNA, 19 nt, synthetic construct
  source 1..19: mol_type = other RNA
tgaccatcagcctcgtgtt

SEQ ID NO: 697 - RNA, 19 nt, synthetic construct
  source 1..19: mol_type = other RNA
gccttaaagaagtagctaa

SEQ ID NO: 698 - RNA, 19 nt, synthetic construct
  source 1..19: mol_type = other RNA
gaagtagctaaggaacttt

SEQ ID NO: 699 - RNA, 19 nt, synthetic construct
  source 1..19: mol_type = other RNA
aaggaactttacatcctaa

SEQ ID NO: 700 - RNA, 19 nt, synthetic construct
  source 1..19: mol_type = other RNA
tgtgtggactaagatgcat

SEQ ID NO: 701 - RNA, 19 nt, synthetic construct
  source 1..19: mol_type = other RNA
gaggaagggaaaacagaaa

SEQ ID NO: 702 - RNA, 19 nt, synthetic construct
  source 1..19: mol_type = other RNA
cccaaaatcccaaactcaa

SEQ ID NO: 703 - RNA, 19 nt, synthetic construct
  source 1..19: mol_type = other RNA
gctgttgattgaagatgta

SEQ ID NO: 704 - RNA, 19 nt, synthetic construct
  source 1..19: mol_type = other RNA
gccttaaagaagtagctaa

SEQ ID NO: 705 - RNA, 19 nt, synthetic construct
  source 1..19: mol_type = other RNA
atcccaaactcaaaccaaa

SEQ ID NO: 706 - RNA, 19 nt, synthetic construct
  source 1..19: mol_type = other RNA
ctgttgattgaagatgtat

SEQ ID NO: 707 - RNA, 19 nt, synthetic construct
  source 1..19: mol_type = other RNA
gtttataaaacctatttat

SEQ ID NO: 708 - RNA, 19 nt, synthetic construct
  source 1..19: mol_type = other RNA
acatagtattgtttgcttt

SEQ ID NO: 709 - RNA, 19 nt, synthetic construct
  source 1..19: mol_type = other RNA
tgaccatcagcctcgtgtt

SEQ ID NO: 710 - RNA, 19 nt, synthetic construct
  source 1..19: mol_type = other RNA
gaagtagctaaggaacttt

SEQ ID NO: 711 - RNA, 19 nt, synthetic construct
  source 1..19: mol_type = other RNA
aaggaactttacatcctaa

SEQ ID NO: 712 - RNA, 19 nt, synthetic construct
  source 1..19: mol_type = other RNA
tgtgtggactaagatgcat

SEQ ID NO: 713 - RNA, 19 nt, synthetic construct
  source 1..19: mol_type = other RNA
ggctctgttcctgttcttc

SEQ ID NO: 714 - RNA, 19 nt, synthetic construct
  source 1..19: mol_type = other RNA
acctatttataacactttt

SEQ ID NO: 715 - RNA, 19 nt, synthetic construct
  source 1..19: mol_type = other RNA
acaataaataaatctcaaa

SEQ ID NO: 716 - RNA, 19 nt, synthetic construct
  source 1..19: mol_type = other RNA
cctcgtgttgaatcttaaa

SEQ ID NO: 717 - RNA, 19 nt, synthetic construct
  source 1..19: mol_type = other RNA
ccaccaactgtagatgtat

SEQ ID NO: 718 - RNA, 19 nt, synthetic construct
  source 1..19: mol_type = other RNA
ctgtccaggccaccatgat

SEQ ID NO: 719 - RNA, 19 nt, synthetic construct
  source 1..19: mol_type = other RNA
ataccaactgtgtggacta

SEQ ID NO: 720 - RNA, 19 nt, synthetic construct
  source 1..19: mol_type = other RNA
aaacctatttataacactt

SEQ ID NO: 721 - RNA, 19 nt, synthetic construct
  source 1..19: mol_type = other RNA
ctcctgttgctgagtatca

SEQ ID NO: 722 - RNA, 19 nt, synthetic construct
  source 1..19: mol_type = other RNA
ctgttgctgagtatcatcg

SEQ ID NO: 723 - RNA, 19 nt, synthetic construct
  source 1..19: mol_type = other RNA
gttgctgagtatcatcgtc

SEQ ID NO: 724 - RNA, 19 nt, synthetic construct
  source 1..19: mol_type = other RNA
gtatcatcgtcctccacgt

SEQ ID NO: 725 - RNA, 19 nt, synthetic construct
  source 1..19: mol_type = other RNA
tcgcggtgctggtgctgct

SEQ ID NO: 726 - RNA, 19 nt, synthetic construct
  source 1..19: mol_type = other RNA
ctggtgctgctgttcgtct

SEQ ID NO: 727 - RNA, 19 nt, synthetic construct
  source 1..19: mol_type = other RNA
ggacacgcaactgatctct

SEQ ID NO: 728 - RNA, 19 nt, synthetic construct
  source 1..19: mol_type = other RNA
gaaatgtccaccactgttt

SEQ ID NO: 729 - RNA, 19 nt, synthetic construct
  source 1..19: mol_type = other RNA
caccactgtttctcatcat

SEQ ID NO: 730 - RNA, 19 nt, synthetic construct
  source 1..19: mol_type = other RNA
acgaatggctgcagtctgt

SEQ ID NO: 731 - RNA, 19 nt, synthetic construct
  source 1..19: mol_type = other RNA
tgttcttctgccaactctt

SEQ ID NO: 732 - RNA, 19 nt, synthetic construct
  source 1..19: mol_type = other RNA
ctgtttggccgggcagaaa

SEQ ID NO: 733 - RNA, 19 nt, synthetic construct
  source 1..19: mol_type = other RNA
gaaactccgctgagcagaa

SEQ ID NO: 734 - RNA, 19 nt, synthetic construct
  source 1..19: mol_type = other RNA
actccgctgagcagaactt

SEQ ID NO: 735 - RNA, 19 nt, synthetic construct
  source 1..19: mol_type = other RNA
aactcttcaccctcaccaa

SEQ ID NO: 736 - RNA, 19 nt, synthetic construct
  source 1..19: mol_type = other RNA
gggcaggttttacatcact

SEQ ID NO: 737 - RNA, 19 nt, synthetic construct
  source 1..19: mol_type = other RNA
ggttttacatcactggaat

SEQ ID NO: 738 - RNA, 19 nt, synthetic construct
  source 1..19: mol_type = other RNA
cactggaatcttccaaatt

SEQ ID NO: 739 - RNA, 19 nt, synthetic construct
  source 1..19: mol_type = other RNA
aatcttccaaattcttgct

SEQ ID NO: 740 - RNA, 19 nt, synthetic construct
  source 1..19: mol_type = other RNA
tgatgagtgctgcggccat

SEQ ID NO: 741 - RNA, 19 nt, synthetic construct
  source 1..19: mol_type = other RNA
acccggagtggcatctcaa

SEQ ID NO: 742 - RNA, 19 nt, synthetic construct
  source 1..19: mol_type = other RNA
cggattactcctacggttt

SEQ ID NO: 743 - RNA, 19 nt, synthetic construct
  source 1..19: mol_type = other RNA
cctacggtttcgcctacat

SEQ ID NO: 744 - RNA, 19 nt, synthetic construct
  source 1..19: mol_type = other RNA
acggtttcgcctacatcct

SEQ ID NO: 745 - RNA, 19 nt, synthetic construct
  source 1..19: mol_type = other RNA
gcggtgtcatctatgtgat

SEQ ID NO: 746 - RNA, 19 nt, synthetic construct
  source 1..19: mol_type = other RNA
gtgtcatctatgtgatctt

SEQ ID NO: 747 - RNA, 19 nt, synthetic construct
  source 1..19: mol_type = other RNA
ctatgtgatcttgcggaaa

SEQ ID NO: 748 - RNA, 21 nt, synthetic construct
  source 1..21: mol_type = other RNA
gggaggaagggaaaacagaaa

SEQ ID NO: 749 - RNA, 21 nt, synthetic construct
  source 1..21: mol_type = other RNA
agcccaaaatcccaaactcaa

SEQ ID NO: 750 - RNA, 21 nt, synthetic construct
  source 1..21: mol_type = other RNA
ttgctgttgattgaagatgta

SEQ ID NO: 751 - RNA, 21 nt, synthetic construct
  source 1..21: mol_type = other RNA
gagccttaaagaagtagctaa

SEQ ID NO: 752 - RNA, 21 nt, synthetic construct
  source 1..21: mol_type = other RNA
gggaggaagggaaaacagaaa

SEQ ID NO: 753 - RNA, 21 nt, synthetic construct
  source 1..21: mol_type = other RNA
agcccaaaatcccaaactcaa

SEQ ID NO: 754 - RNA, 21 nt, synthetic construct
  source 1..21: mol_type = other RNA
ttgctgttgattgaagatgta

SEQ ID NO: 755 - RNA, 21 nt, synthetic construct
  source 1..21: mol_type = other RNA
gagccttaaagaagtagctaa

SEQ ID NO: 756 - RNA, 21 nt, synthetic construct
  source 1..21: mol_type = other RNA
ctttacatcctaacagtataa

SEQ ID NO: 757 - RNA, 21 nt, synthetic construct
  source 1..21: mol_type = other RNA
ttttacccagaaataagataa

SEQ ID NO: 758 - RNA, 21 nt, synthetic construct
  source 1..21: mol_type = other RNA
ggccaccaactgtagatgtat

SEQ ID NO: 759 - RNA, 21 nt, synthetic construct
  source 1..21: mol_type = other RNA
tttgctttgcattttctgatt

SEQ ID NO: 760 - RNA, 21 nt, synthetic construct
  source 1..21: mol_type = other RNA
accaactgtgtggactaagat

SEQ ID NO: 761 - RNA, 19 nt, synthetic construct
  source 1..19: mol_type = other RNA
ttacatcctaacagtataa

SEQ ID NO: 762 - RNA, 19 nt, synthetic construct
  source 1..19: mol_type = other RNA
ttacccagaaataagataa

SEQ ID NO: 763 - RNA, 19 nt, synthetic construct
  source 1..19: mol_type = other RNA
tgctttgcattttctgatt

SEQ ID NO: 764 - RNA, 19 nt, synthetic construct
  source 1..19: mol_type = other RNA
caactgtgtggactaagat

SEQ ID NO: 765 - RNA, 21 nt, synthetic construct
  source 1..21: mol_type = other RNA
ctttacatcctaacagtataa

SEQ ID NO: 766 - RNA, 21 nt, synthetic construct
  source 1..21: mol_type = other RNA
ttttacccagaaataagataa

SEQ ID NO: 767 - RNA, 21 nt, synthetic construct
  source 1..21: mol_type = other RNA
ggccaccaactgtagatgtat

SEQ ID NO: 768 - RNA, 21 nt, synthetic construct
  source 1..21: mol_type = other RNA
tttgctttgcattttctgatt

SEQ ID NO: 769 - RNA, 21 nt, synthetic construct
  source 1..21: mol_type = other RNA
accaactgtgtggactaagat

SEQ ID NO: 770 - RNA, 21 nt, synthetic construct
  source 1..21: mol_type = other RNA
cctcctgttgctgagtatcat

SEQ ID NO: 771 - RNA, 21 nt, synthetic construct
  source 1..21: mol_type = other RNA
cctcctgttgctgagtatcat

SEQ ID NO: 772 - RNA, 21 nt, synthetic construct
  source 1..21: mol_type = other RNA
cctcctgttgctgagtatcat

SEQ ID NO: 773 - RNA, 21 nt, synthetic construct
  source 1..21: mol_type = other RNA
cctcctgttgctgagtatcat

SEQ ID NO: 774 - RNA, 21 nt, synthetic construct
  source 1..21: mol_type = other RNA
cctcctgttgctgagtatcat

SEQ ID NO: 775 - RNA, 21 nt, synthetic construct
  source 1..21: mol_type = other RNA
cctcctgttgctgagtatcat

SEQ ID NO: 776 - RNA, 21 nt, synthetic construct
  source 1..21: mol_type = other RNA
cctcctgttgctgagtatcat

SEQ ID NO: 777 - RNA, 21 nt, synthetic construct
  source 1..21: mol_type = other RNA
cctcctgttgctgagtatcat

SEQ ID NO: 778 - RNA, 21 nt, synthetic construct
  source 1..21: mol_type = other RNA
cctcctgttgctgagtatcat

SEQ ID NO: 779 - RNA, 21 nt, synthetic construct
  source 1..21: mol_type = other RNA
cctcctgttgctgagtatcat

SEQ ID NO: 780 - RNA, 21 nt, synthetic construct
  source 1..21: mol_type = other RNA
tcctgttgctgagtatcatcg

SEQ ID NO: 781 - RNA, 21 nt, synthetic construct
  source 1..21: mol_type = other RNA
ctgttgctgagtatcatcgtc

SEQ ID NO: 782 - RNA, 21 nt, synthetic construct
  source 1..21: mol_type = other RNA
tgttgctgagtatcatcgtcc

SEQ ID NO: 783 - RNA, 21 nt, synthetic construct
  source 1..21: mol_type = other RNA
ggcaatggacacgcaactgat

SEQ ID NO: 784 - RNA, 21 nt, synthetic construct
  source 1..21: mol_type = other RNA
ggccaccatgatcctgtcgat

SEQ ID NO: 785 - RNA, 21 nt, synthetic construct
  source 1..21: mol_type = other RNA
cctgtcgatcatcttcagcat

SEQ ID NO: 786 - RNA, 21 nt, synthetic construct
  source 1..21: mol_type = other RNA
cctcctgttgctgggtatcat

SEQ ID NO: 787 - RNA, 21 nt, synthetic construct
  source 1..21: mol_type = other RNA
atgctcctcctgttgctgagt

SEQ ID NO: 788 - RNA, 21 nt, synthetic construct
  source 1..21: mol_type = other RNA
gctcctcctgttgctgagtat

SEQ ID NO: 789 - RNA, 21 nt, synthetic construct
  source 1..21: mol_type = other RNA
ctcctcctgttgctgagtatc

SEQ ID NO: 790 - RNA, 21 nt, synthetic construct
  source 1..21: mol_type = other RNA
tcctcctgttgctgagtatca

SEQ ID NO: 791 - RNA, 21 nt, synthetic construct
  source 1..21: mol_type = other RNA
ctcctgttgctgagtatcatc

SEQ ID NO: 792 - RNA, 21 nt, synthetic construct
  source 1..21: mol_type = other RNA
tcctgttgctgagtatcatcg

SEQ ID NO: 793 - RNA, 21 nt, synthetic construct
  source 1..21: mol_type = other RNA
ctgttgctgagtatcatcgtc

SEQ ID NO: 794 - RNA, 21 nt, synthetic construct
  source 1..21: mol_type = other RNA
tgttgctgagtatcatcgtcc

SEQ ID NO: 795 - RNA, 21 nt, synthetic construct
  source 1..21: mol_type = other RNA
atgctcctcctgttgctgagt

SEQ ID NO: 796 - RNA, 21 nt, synthetic construct
  source 1..21: mol_type = other RNA
gctcctcctgttgctgagtat

SEQ ID NO: 797 - RNA, 21 nt, synthetic construct
  source 1..21: mol_type = other RNA
ctcctcctgttgctgagtatc

SEQ ID NO: 798 - RNA, 21 nt, synthetic construct
  source 1..21: mol_type = other RNA
tcctcctgttgctgagtatca

SEQ ID NO: 799 - RNA, 21 nt, synthetic construct
  source 1..21: mol_type = other RNA
ctcctgttgctgagtatcatc

SEQ ID NO: 800 - RNA, 21 nt, synthetic construct
  source 1..21: mol_type = other RNA
gagtatcatcgtcctccacgt

SEQ ID NO: 801 - RNA, 21 nt, synthetic construct
  source 1..21: mol_type = other RNA
cgtcgcggtgctggtgctgct

SEQ ID NO: 802 - RNA, 21 nt, synthetic construct
  source 1..21: mol_type = other RNA
tgctggtgctgctgttcgtct

SEQ ID NO: 803 - RNA, 21 nt, synthetic construct
  source 1..21: mol_type = other RNA
ggcaatggacacgcaactgat

SEQ ID NO: 804 - RNA, 21 nt, synthetic construct
  source 1..21: mol_type = other RNA
atggacacgcaactgatctct

SEQ ID NO: 805 - RNA, 21 nt, synthetic construct
  source 1..21: mol_type = other RNA
aggaaatgtccaccactgttt

SEQ ID NO: 806 - RNA, 21 nt, synthetic construct
  source 1..21: mol_type = other RNA
tccaccactgtttctcatcat

SEQ ID NO: 807 - RNA, 21 nt, synthetic construct
  source 1..21: mol_type = other RNA
aaacgaatggctgcagtctgt

SEQ ID NO: 808 - RNA, 21 nt, synthetic construct
  source 1..21: mol_type = other RNA
ggccaccatgatcctgtcgat

SEQ ID NO: 809 - RNA, 21 nt, synthetic construct
  source 1..21: mol_type = other RNA
cctgtcgatcatcttcagcat

SEQ ID NO: 810 - RNA, 21 nt, synthetic construct
  source 1..21: mol_type = other RNA
gttcctgttcttctgccaact

SEQ ID NO: 811 - RNA, 21 nt, synthetic construct
  source 1..21: mol_type = other RNA
cctgttcttctgccaactctt

SEQ ID NO: 812 - RNA, 21 nt, synthetic construct
  source 1..21: mol_type = other RNA
gagtatcatcgtcctccacgt

SEQ ID NO: 813 - RNA, 21 nt, synthetic construct
  source 1..21: mol_type = other RNA
cgtcgcggtgctggtgctgct

SEQ ID NO: 814 - RNA, 21 nt, synthetic construct
  source 1..21: mol_type = other RNA
tgctggtgctgctgttcgtct

SEQ ID NO: 815 - RNA, 21 nt, synthetic construct
  source 1..21: mol_type = other RNA
atggacacgcaactgatctct

SEQ ID NO: 816 - RNA, 21 nt, synthetic construct
  source 1..21: mol_type = other RNA
aggaaatgtccaccactgttt

SEQ ID NO: 817 - RNA, 21 nt, synthetic construct
  source 1..21: mol_type = other RNA
tccaccactgtttctcatcat

SEQ ID NO: 818 - RNA, 21 nt, synthetic construct
  source 1..21: mol_type = other RNA
aaacgaatggctgcagtctgt

SEQ ID NO: 819 - RNA, 21 nt, synthetic construct
  source 1..21: mol_type = other RNA
gttcctgttcttctgccaact

SEQ ID NO: 820 - RNA, 21 nt, synthetic construct
  source 1..21: mol_type = other RNA
cctgttcttctgccaactctt

SEQ ID NO: 821 - RNA, 21 nt, synthetic construct
  source 1..21: mol_type = other RNA
ccaactcttcaccctcaccaa

SEQ ID NO: 822 - RNA, 21 nt, synthetic construct
  source 1..21: mol_type = other RNA
gggggcaggttttacatcact

SEQ ID NO: 823 - RNA, 21 nt, synthetic construct
  source 1..21: mol_type = other RNA
caggttttacatcactggaat

SEQ ID NO: 824 - RNA, 21 nt, synthetic construct
  source 1..21: mol_type = other RNA
gttttacatcactggaatctt

SEQ ID NO: 825 - RNA, 21 nt, synthetic construct
  source 1..21: mol_type = other RNA
atcactggaatcttccaaatt

SEQ ID NO: 826 - RNA, 21 nt, synthetic construct
  source 1..21: mol_type = other RNA
ggaatcttccaaattcttgct

SEQ ID NO: 827 - RNA, 21 nt, synthetic construct
  source 1..21: mol_type = other RNA
cgtgatgagtgctgcggccat

SEQ ID NO: 828 - RNA, 21 nt, synthetic construct
  source 1..21: mol_type = other RNA
gcacccggagtggcatctcaa

SEQ ID NO: 829 - RNA, 21 nt, synthetic construct
  source 1..21: mol_type = other RNA
ctcggattactcctacggttt

SEQ ID NO: 830 - RNA, 21 nt, synthetic construct
  source 1..21: mol_type = other RNA
ctcctacggtttcgcctacat

SEQ ID NO: 831 - RNA, 21 nt, synthetic construct
  source 1..21: mol_type = other RNA
ctacggtttcgcctacatcct

SEQ ID NO: 832 - RNA, 21 nt, synthetic construct
  source 1..21: mol_type = other RNA
cagcggtgtcatctatgtgat

SEQ ID NO: 833 - RNA, 21 nt, synthetic construct
  source 1..21: mol_type = other RNA
cggtgtcatctatgtgatctt

SEQ ID NO: 834 - RNA, 21 nt, synthetic construct
  source 1..21: mol_type = other RNA
atctatgtgatcttgcggaaa

SEQ ID NO: 835 - RNA, 21 nt, synthetic construct
  source 1..21: mol_type = other RNA
ccaactcttcaccctcaccaa

SEQ ID NO: 836 - RNA, 21 nt, synthetic construct
  source 1..21: mol_type = other RNA
gggggcaggttttacatcact

SEQ ID NO: 837 - RNA, 21 nt, synthetic construct
  source 1..21: mol_type = other RNA
caggttttacatcactggaat

SEQ ID NO: 838 - RNA, 21 nt, synthetic construct
  source 1..21: mol_type = other RNA
gttttacatcactggaatctt

SEQ ID NO: 839 - RNA, 21 nt, synthetic construct
  source 1..21: mol_type = other RNA
atcactggaatcttccaaatt

SEQ ID NO: 840 - RNA, 21 nt, synthetic construct
  source 1..21: mol_type = other RNA
ggaatcttccaaattcttgct

SEQ ID NO: 841 - RNA, 21 nt, synthetic construct
  source 1..21: mol_type = other RNA
cgtgatgagtgctgcggccat

SEQ ID NO: 842 - RNA, 21 nt, synthetic construct
  source 1..21: mol_type = other RNA
gcacccggagtggcatctcaa

SEQ ID NO: 843 - RNA, 21 nt, synthetic construct
  source 1..21: mol_type = other RNA
ctcggattactcctacggttt

SEQ ID NO: 844 - RNA, 21 nt, synthetic construct
  source 1..21: mol_type = other RNA
ctcctacggtttcgcctacat

SEQ ID NO: 845 - RNA, 21 nt, synthetic construct
  source 1..21: mol_type = other RNA
ctacggtttcgcctacatcct

SEQ ID NO: 846 - RNA, 21 nt, synthetic construct
  source 1..21: mol_type = other RNA
cagcggtgtcatctatgtgat

SEQ ID NO: 847 - RNA, 21 nt, synthetic construct
  source 1..21: mol_type = other RNA
cggtgtcatctatgtgatctt

SEQ ID NO: 848 - RNA, 21 nt, synthetic construct
  source 1..21: mol_type = other RNA
atctatgtgatcttgcggaaa

SEQ ID NO: 849 - RNA, 21 nt, synthetic construct
  source 1..21: mol_type = other RNA
aaatcccaaactcaaaccaaa

SEQ ID NO: 850 - RNA, 21 nt, synthetic construct
  source 1..21: mol_type = other RNA
tgctgttgattgaagatgtat

SEQ ID NO: 851 - RNA, 21 nt, synthetic construct
  source 1..21: mol_type = other RNA
cggtttataaaacctatttat

SEQ ID NO: 852 - RNA, 21 nt, synthetic construct
  source 1..21: mol_type = other RNA
gtacatagtattgtttgcttt

SEQ ID NO: 853 - RNA, 21 nt, synthetic construct
  source 1..21: mol_type = other RNA
gttgaccatcagcctcgtgtt

SEQ ID NO: 854 - RNA, 21 nt, synthetic construct
  source 1..21: mol_type = other RNA
aagaagtagctaaggaacttt

SEQ ID NO: 855 - RNA, 21 nt, synthetic construct
  source 1..21: mol_type = other RNA
ctaaggaactttacatcctaa

SEQ ID NO: 856 - RNA, 21 nt, synthetic construct
  source 1..21: mol_type = other RNA
actgtgtggactaagatgcat

SEQ ID NO: 857 - RNA, 21 nt, synthetic construct
  source 1..21: mol_type = other RNA
aaatcccaaactcaaaccaaa

SEQ ID NO: 858 - RNA, 21 nt, synthetic construct
  source 1..21: mol_type = other RNA
tgctgttgattgaagatgtat

SEQ ID NO: 859 - RNA, 21 nt, synthetic construct
  source 1..21: mol_type = other RNA
cggtttataaaacctatttat

SEQ ID NO: 860 - RNA, 21 nt, synthetic construct
  source 1..21: mol_type = other RNA
gtacatagtattgtttgcttt

SEQ ID NO: 861 - RNA, 21 nt, synthetic construct
  source 1..21: mol_type = other RNA
gttgaccatcagcctcgtgtt

SEQ ID NO: 862 - RNA, 21 nt, synthetic construct
  source 1..21: mol_type = other RNA
aagaagtagctaaggaacttt

SEQ ID NO: 863 - RNA, 21 nt, synthetic construct
  source 1..21: mol_type = other RNA
ctaaggaactttacatcctaa

SEQ ID NO: 864 - RNA, 21 nt, synthetic construct
  source 1..21: mol_type = other RNA
actgtgtggactaagatgcat

SEQ ID NO: 865 - RNA, 21 nt, synthetic construct
  source 1..21: mol_type = other RNA
cgctgtttggccgggcagaaa

SEQ ID NO: 866 - RNA, 21 nt, synthetic construct
  source 1..21: mol_type = other RNA
cagaaactccgctgagcagaa

SEQ ID NO: 867 - RNA, 21 nt, synthetic construct
  source 1..21: mol_type = other RNA
aaactccgctgagcagaactt

SEQ ID NO: 868 - RNA, 21 nt, synthetic construct
  source 1..21: mol_type = other RNA
cgctgtttggccgggcagaaa

SEQ ID NO: 869 - RNA, 21 nt, synthetic construct
  source 1..21: mol_type = other RNA
cagaaactccgctgagcagaa

SEQ ID NO: 870 - RNA, 21 nt, synthetic construct
  source 1..21: mol_type = other RNA
aaactccgctgagcagaactt

SEQ ID NO: 871 - RNA, 21 nt, synthetic construct
  source 1..21: mol_type = other RNA
cggtgtcatctatgtgatctt

SEQ ID NO: 872 - RNA, 21 nt, synthetic construct
  source 1..21: mol_type = other RNA
ctcctcctgttgctgagtatt

SEQ ID NO: 873 - RNA, 21 nt, synthetic construct
  source 1..21: mol_type = other RNA
ctcctcctgttgctgagtatt

SEQ ID NO: 874 - RNA, 21 nt, synthetic construct
  source 1..21: mol_type = other RNA
ctcctgttgctgagtatcatt

SEQ ID NO: 875 - RNA, 21 nt, synthetic construct
  source 1..21: mol_type = other RNA
ctcctgttgctgagtatcatt

SEQ ID NO: 876 - RNA, 21 nt, synthetic construct
  source 1..21: mol_type = other RNA
gagtatcatcgtcctccacgt

SEQ ID NO: 877 - RNA, 21 nt, synthetic construct
  source 1..21: mol_type = other RNA
cctcctgttgctgagtatcat

SEQ ID NO: 878 - RNA, 21 nt, synthetic construct
  source 1..21: mol_type = other RNA
cctcctgttgctgagtatcat

SEQ ID NO: 879 - RNA, 23 nt, synthetic construct
  source 1..23: mol_type = other RNA
atgatactcagcaacaggaggag

SEQ ID NO: 880 - RNA, 23 nt, synthetic construct
  source 1..23: mol_type = other RNA
atgatactcagcaacaggaggag

SEQ ID NO: 881 - RNA, 23 nt, synthetic construct
  source 1..23: mol_type = other RNA
tttctgttttcccttcctccctt

SEQ ID NO: 882 - RNA, 23 nt, synthetic construct
  source 1..23: mol_type = other RNA
ttgagtttgggattttgggctcg

SEQ ID NO: 883 - RNA, 23 nt, synthetic construct
  source 1..23: mol_type = other RNA
tacatcttcaatcaacagcaacc

SEQ ID NO: 884 - RNA, 23 nt, synthetic construct
  source 1..23: mol_type = other RNA
ttagctacttctttaaggctcaa

SEQ ID NO: 885 - RNA, 23 nt, synthetic construct
  source 1..23: mol_type = other RNA
tttctgttttcccttcctccctt

SEQ ID NO: 886 - RNA, 23 nt, synthetic construct
  source 1..23: mol_type = other RNA
ttgagtttgggattttgggctcg

SEQ ID NO: 887 - RNA, 23 nt, synthetic construct
  source 1..23: mol_type = other RNA
tacatcttcaatcaacagcaacc

SEQ ID NO: 888 - RNA, 23 nt, synthetic construct
  source 1..23: mol_type = other RNA
ttagctacttctttaaggctcaa

SEQ ID NO: 889 - RNA, 23 nt, synthetic construct
  source 1..23: mol_type = other RNA
ttatactgttaggatgtaaagtt

SEQ ID NO: 890 - RNA, 23 nt, synthetic construct
  source 1..23: mol_type = other RNA
ttatcttatttctgggtaaaaca

SEQ ID NO: 891 - RNA, 23 nt, synthetic construct
  source 1..23: mol_type = other RNA
atacatctacagttggtggccaa

SEQ ID NO: 892 - RNA, 23 nt, synthetic construct
  source 1..23: mol_type = other RNA
aatcagaaaatgcaaagcaaaaa

SEQ ID NO: 893 - RNA, 23 nt, synthetic construct
  source 1..23: mol_type = other RNA
atcttagtccacacagttggtat

SEQ ID NO: 894 - RNA, 23 nt, synthetic construct
  source 1..23: mol_type = other RNA
ttatactgttaggatgtaaagtt

SEQ ID NO: 895 - RNA, 23 nt, synthetic construct
  source 1..23: mol_type = other RNA
ttatcttatttctgggtaaaaca

SEQ ID NO: 896 - RNA, 23 nt, synthetic construct
  source 1..23: mol_type = other RNA
atacatctacagttggtggccaa

SEQ ID NO: 897 - RNA, 23 nt, synthetic construct
  source 1..23: mol_type = other RNA
aatcagaaaatgcaaagcaaaaa

SEQ ID NO: 898 - RNA, 23 nt, synthetic construct
  source 1..23: mol_type = other RNA
atcttagtccacacagttggtat

SEQ ID NO: 899 - RNA, 23 nt, synthetic construct
  source 1..23: mol_type = other RNA
atgatactcagcaacaggaggag

SEQ ID NO: 900 - RNA, 23 nt, synthetic construct
  source 1..23: mol_type = other RNA
atgatactcagcaacaggaggag

SEQ ID NO: 901 - RNA, 23 nt, synthetic construct
  source 1..23: mol_type = other RNA
atgatactcagcaacaggaggag

SEQ ID NO: 902 - RNA, 23 nt, synthetic construct
  source 1..23: mol_type = other RNA
atgatactcagcaacaggaggag

SEQ ID NO: 903 - RNA, 23 nt, synthetic construct
  source 1..23: mol_type = other RNA
cgatgatactcagcaacaggagg

SEQ ID NO: 904 - RNA, 23 nt, synthetic construct
  source 1..23: mol_type = other RNA
gacgatgatactcagcaacagga

SEQ ID NO: 905 - RNA, 23 nt, synthetic construct
  source 1..23: mol_type = other RNA
ggacgatgatactcagcaacagg

SEQ ID NO: 906 - RNA, 23 nt, synthetic construct
  source 1..23: mol_type = other RNA
atcagttgcgtgtccattgccca

SEQ ID NO: 907 - RNA, 23 nt, synthetic construct
  source 1..23: mol_type = other RNA
atcgacaggatcatggtggcctg

SEQ ID NO: 908 - RNA, 23 nt, synthetic construct
  source 1..23: mol_type = other RNA
atgctgaagatgatcgacaggat

SEQ ID NO: 909 - RNA, 23 nt, synthetic construct
  source 1..23: mol_type = other RNA
atgatacccagcaacaggaggag

SEQ ID NO: 910 - RNA, 23 nt, synthetic construct
  source 1..23: mol_type = other RNA
actcagcaacaggaggagcattc

SEQ ID NO: 911 - RNA, 23 nt, synthetic construct
  source 1..23: mol_type = other RNA
atactcagcaacaggaggagcat

SEQ ID NO: 912 - RNA, 23 nt, synthetic construct
  source 1..23: mol_type = other RNA
gatactcagcaacaggaggagca

SEQ ID NO: 913 - RNA, 23 nt, synthetic construct
  source 1..23: mol_type = other RNA
tgatactcagcaacaggaggagc

SEQ ID NO: 914 - RNA, 23 nt, synthetic construct
  source 1..23: mol_type = other RNA
gatgatactcagcaacaggagga

SEQ ID NO: 915 - RNA, 23 nt, synthetic construct
  source 1..23: mol_type = other RNA
cgatgatactcagcaacaggagg

SEQ ID NO: 916 - RNA, 23 nt, synthetic construct
  source 1..23: mol_type = other RNA
gacgatgatactcagcaacagga

SEQ ID NO: 917 - RNA, 23 nt, synthetic construct
  source 1..23: mol_type = other RNA
ggacgatgatactcagcaacagg

SEQ ID NO: 918 - RNA, 23 nt, synthetic construct
  source 1..23: mol_type = other RNA
acgtggaggacgatgatactcag

SEQ ID NO: 919 - RNA, 23 nt, synthetic construct
  source 1..23: mol_type = other RNA
agcagcaccagcaccgcgacgtg

SEQ ID NO: 920 - RNA, 23 nt, synthetic construct
  source 1..23: mol_type = other RNA
agacgaacagcagcaccagcacc

SEQ ID NO: 921 - RNA, 23 nt, synthetic construct
  source 1..23: mol_type = other RNA
atcagttgcgtgtccattgccca

SEQ ID NO: 922 - RNA, 23 nt, synthetic construct
  source 1..23: mol_type = other RNA
agagatcagttgcgtgtccattc

SEQ ID NO: 923 - RNA, 23 nt, synthetic construct
  source 1..23: mol_type = other RNA
aaacagtggtggacatttcctga

SEQ ID NO: 924 - RNA, 23 nt, synthetic construct
  source 1..23: mol_type = other RNA
atgatgagaaacagtggtggaca

SEQ ID NO: 925 - RNA, 23 nt, synthetic construct
  source 1..23: mol_type = other RNA
acagactgcagccattcgtttgg

SEQ ID NO: 926 - RNA, 23 nt, synthetic construct
  source 1..23: mol_type = other RNA
atcgacaggatcatggtggcctg

SEQ ID NO: 927 - RNA, 23 nt, synthetic construct
  source 1..23: mol_type = other RNA
atgctgaagatgatcgacaggat

SEQ ID NO: 928 - RNA, 23 nt, synthetic construct
  source 1..23: mol_type = other RNA
agttggcagaagaacaggaacag

SEQ ID NO: 929 - RNA, 23 nt, synthetic construct
  source 1..23: mol_type = other RNA
aagagttggcagaagaacaggaa

SEQ ID NO: 930 - RNA, 23 nt, synthetic construct
  source 1..23: mol_type = other RNA
ttggtgagggtgaagagttggca

SEQ ID NO: 931 - RNA, 23 nt, synthetic construct
  source 1..23: mol_type = other RNA
agtgatgtaaaacctgcccccct

SEQ ID NO: 932 - RNA, 23 nt, synthetic construct
  source 1..23: mol_type = other RNA
attccagtgatgtaaaacctgcc

SEQ ID NO: 933 - RNA, 23 nt, synthetic construct
  source 1..23: mol_type = other RNA
aagattccagtgatgtaaaacct

SEQ ID NO: 934 - RNA, 23 nt, synthetic construct
  source 1..23: mol_type = other RNA
aatttggaagattccagtgatgt

SEQ ID NO: 935 - RNA, 23 nt, synthetic construct
  source 1..23: mol_type = other RNA
agcaagaatttggaagattccag

SEQ ID NO: 936 - RNA, 23 nt, synthetic construct
  source 1..23: mol_type = other RNA
atggccgcagcactcatcacgca

SEQ ID NO: 937 - RNA, 23 nt, synthetic construct
  source 1..23: mol_type = other RNA
ttgagatgccactccgggtgcct

SEQ ID NO: 938 - RNA, 23 nt, synthetic construct
  source 1..23: mol_type = other RNA
aaaccgtaggagtaatccgagtt

SEQ ID NO: 939 - RNA, 23 nt, synthetic construct
  source 1..23: mol_type = other RNA
atgtaggcgaaaccgtaggagta

SEQ ID NO: 940 - RNA, 23 nt, synthetic construct
  source 1..23: mol_type = other RNA
aggatgtaggcgaaaccgtagga

SEQ ID NO: 941 - RNA, 23 nt, synthetic construct
  source 1..23: mol_type = other RNA
atcacatagatgacaccgctgag

SEQ ID NO: 942 - RNA, 23 nt, synthetic construct
  source 1..23: mol_type = other RNA
aagatcacatagatgacaccgct

SEQ ID NO: 943 - RNA, 23 nt, synthetic construct
  source 1..23: mol_type = other RNA
tttccgcaagatcacatagatga

SEQ ID NO: 944 - RNA, 23 nt, synthetic construct
  source 1..23: mol_type = other RNA
tttggtttgagtttgggattttg

SEQ ID NO: 945 - RNA, 23 nt, synthetic construct
  source 1..23: mol_type = other RNA
atacatcttcaatcaacagcaac

SEQ ID NO: 946 - RNA, 23 nt, synthetic construct
  source 1..23: mol_type = other RNA
ataaataggttttataaaccgga

SEQ ID NO: 947 - RNA, 23 nt, synthetic construct
  source 1..23: mol_type = other RNA
aaagcaaacaatactatgtacat

SEQ ID NO: 948 - RNA, 23 nt, synthetic construct
  source 1..23: mol_type = other RNA
aacacgaggctgatggtcaacat

SEQ ID NO: 949 - RNA, 23 nt, synthetic construct
  source 1..23: mol_type = other RNA
aaagttccttagctacttcttta

SEQ ID NO: 950 - RNA, 23 nt, synthetic construct
  source 1..23: mol_type = other RNA
ttaggatgtaaagttccttagct

SEQ ID NO: 951 - RNA, 23 nt, synthetic construct
  source 1..23: mol_type = other RNA
atgcatcttagtccacacagttg

SEQ ID NO: 952 - RNA, 23 nt, synthetic construct
  source 1..23: mol_type = other RNA
tttctgcccggccaaacagcgta

SEQ ID NO: 953 - RNA, 23 nt, synthetic construct
  source 1..23: mol_type = other RNA
ttctgctcagcggagtttctgcc

SEQ ID NO: 954 - RNA, 23 nt, synthetic construct
  source 1..23: mol_type = other RNA
aagttctgctcagcggagtttct

SEQ ID NO: 955 - RNA, 23 nt, synthetic construct
  source 1..23: mol_type = other RNA
agcagcaccagcaccgcgacgtg

SEQ ID NO: 956 - RNA, 23 nt, synthetic construct
  source 1..23: mol_type = other RNA
agacgaacagcagcaccagcacc

SEQ ID NO: 957 - RNA, 23 nt, synthetic construct
  source 1..23: mol_type = other RNA
aaacagtggtggacatttcctga

SEQ ID NO: 958 - RNA, 23 nt, synthetic construct
  source 1..23: mol_type = other RNA
atgatgagaaacagtggtggaca

SEQ ID NO: 959 - RNA, 23 nt, synthetic construct
  source 1..23: mol_type = other RNA
acagactgcagccattcgtttgg

SEQ ID NO: 960 - RNA, 23 nt, synthetic construct
  source 1..23: mol_type = other RNA
aagagttggcagaagaacaggaa

SEQ ID NO: 961 - RNA, 23 nt, synthetic construct
  source 1..23: mol_type = other RNA
ttggtgagggtgaagagttggca

SEQ ID NO: 962 - RNA, 23 nt, synthetic construct
  source 1..23: mol_type = other RNA
agtgatgtaaaacctgcccccct

SEQ ID NO: 963 - RNA, 23 nt, synthetic construct
  source 1..23: mol_type = other RNA
attccagtgatgtaaaacctgcc

SEQ ID NO: 964 - RNA, 23 nt, synthetic construct
  source 1..23: mol_type = other RNA
aagattccagtgatgtaaaacct

SEQ ID NO: 965 - RNA, 23 nt, synthetic construct
  source 1..23: mol_type = other RNA
atggccgcagcactcatcacgca

SEQ ID NO: 966 - RNA, 23 nt, synthetic construct
  source 1..23: mol_type = other RNA
aaaccgtaggagtaatccgagtt

SEQ ID NO: 967 - RNA, 23 nt, synthetic construct
  source 1..23: mol_type = other RNA
aagatcacatagatgacaccgct

SEQ ID NO: 968 - RNA, 23 nt, synthetic construct
  source 1..23: mol_type = other RNA
aagatcacatagatgacaccgct

SEQ ID NO: 969 - RNA, 23 nt, synthetic construct
  source 1..23: mol_type = other RNA
aatactcagcaacaggaggagca

SEQ ID NO: 970 - RNA, 23 nt, synthetic construct
  source 1..23: mol_type = other RNA
aatactcagcaacaggaggagca

SEQ ID NO: 971 - RNA, 23 nt, synthetic construct
  source 1..23: mol_type = other RNA
aatgatactcagcaacaggagga

SEQ ID NO: 972 - RNA, 23 nt, synthetic construct
  source 1..23: mol_type = other RNA
aatgatactcagcaacaggagga

SEQ ID NO: 973 - RNA, 23 nt, synthetic construct
  source 1..23: mol_type = other RNA
acgtggaggacgatgatactcag

SEQ ID NO: 974 - RNA, 23 nt, synthetic construct
  source 1..23: mol_type = other RNA
acgtggaggacgatgatactcag

SEQ ID NO: 975 - RNA, 23 nt, synthetic construct
  source 1..23: mol_type = other RNA
atgatactcagcaacaggaggag

SEQ ID NO: 976 - RNA, 19 nt, synthetic construct
  source 1..19: mol_type = other RNA
aaacctatttataacactt

SEQ ID NO: 977 - RNA, 21 nt, synthetic construct
  source 1..21: mol_type = other RNA
aaacgaatggctgcagtctgt

SEQ ID NO: 978 - RNA, 21 nt, synthetic construct
  source 1..21: mol_type = other RNA
aaactccgctgagcagaactt

SEQ ID NO: 979 - RNA, 21 nt, synthetic construct
  source 1..21: mol_type = other RNA
aaatcccaaactcaaaccaaa

SEQ ID NO: 980 - RNA, 19 nt, synthetic construct
  source 1..19: mol_type = other RNA
aactcttcaccctcaccaa

SEQ ID NO: 981 - RNA, 21 nt, synthetic construct
  source 1..21: mol_type = other RNA
aagaagtagctaaggaacttt

SEQ ID NO: 982 - RNA, 19 nt, synthetic construct
  source 1..19: mol_type = other RNA
aaggaactttacatcctaa

SEQ ID NO: 983 - RNA, 19 nt, synthetic construct
  source 1..19: mol_type = other RNA
aatcttccaaattcttgct

SEQ ID NO: 984 - RNA, 19 nt, synthetic construct
  source 1..19: mol_type = other RNA
acaataaataaatctcaaa

SEQ ID NO: 985 - RNA, 19 nt, synthetic construct
  source 1..19: mol_type = other RNA
acatagtattgtttgcttt

SEQ ID NO: 986 - RNA, 21 nt, synthetic construct
  source 1..21: mol_type = other RNA
accaactgtgtggactaagat

SEQ ID NO: 987 - RNA, 19 nt, synthetic construct
  source 1..19: mol_type = other RNA
acccggagtggcatctcaa

SEQ ID NO: 988 - RNA, 19 nt, synthetic construct
  source 1..19: mol_type = other RNA
acctatttataacactttt

SEQ ID NO: 989 - RNA, 19 nt, synthetic construct
  source 1..19: mol_type = other RNA
acgaatggctgcagtctgt

SEQ ID NO: 990 - RNA, 19 nt, synthetic construct
  source 1..19: mol_type = other RNA
acggtttcgcctacatcct

SEQ ID NO: 991 - RNA, 19 nt, synthetic construct
  source 1..19: mol_type = other RNA
actccgctgagcagaactt

SEQ ID NO: 992 - RNA, 21 nt, synthetic construct
  source 1..21: mol_type = other RNA
actgtgtggactaagatgcat

SEQ ID NO: 993 - RNA, 21 nt, synthetic construct
  source 1..21: mol_type = other RNA
agcccaaaatcccaaactcaa

SEQ ID NO: 994 - RNA, 21 nt, synthetic construct
  source 1..21: mol_type = other RNA
aggaaatgtccaccactgttt

SEQ ID NO: 995 - RNA, 19 nt, synthetic construct
  source 1..19: mol_type = other RNA
ataccaactgtgtggacta

SEQ ID NO: 996 - RNA, 19 nt, synthetic construct
  source 1..19: mol_type = other RNA
atcactggaatcttccaaa

SEQ ID NO: 997 - RNA, 21 nt, synthetic construct
  source 1..21: mol_type = other RNA
atcactggaatcttccaaatt

SEQ ID NO: 998 - RNA, 19 nt, synthetic construct
  source 1..19: mol_type = other RNA
atcccaaactcaaaccaaa

SEQ ID NO: 999 - RNA, 21 nt, synthetic construct
  source 1..21: mol_type = other RNA
atctatgtgatcttgcggaaa

SEQ ID NO: 1000 - RNA, 21 nt, synthetic construct
  source 1..21: mol_type = other RNA
atgctcctcctgttgctgagt

SEQ ID NO: 1001 - RNA, 21 nt, synthetic construct
  source 1..21: mol_type = other RNA
atggacacgcaactgatctct

SEQ ID NO: 1002 - RNA, 19 nt, synthetic construct
  source 1..19: mol_type = other RNA
caaattcttgctggtctgt

SEQ ID NO: 1003 - RNA, 19 nt, synthetic construct
  source 1..19: mol_type = other RNA
caactgtgtggactaagat

SEQ ID NO: 1004 - RNA, 19 nt, synthetic construct
  source 1..19: mol_type = other RNA
caatggacacgcaactgat

SEQ ID NO: 1005 - RNA, 19 nt, synthetic construct
  source 1..19: mol_type = other RNA
caccactgtttctcatcat

SEQ ID NO: 1006 - RNA, 19 nt, synthetic construct
  source 1..19: mol_type = other RNA
cactggaatcttccaaatt

SEQ ID NO: 1007 - RNA, 21 nt, synthetic construct
  source 1..21: mol_type = other RNA
cagaaactccgctgagcagaa

SEQ ID NO: 1008 - RNA, 21 nt, synthetic construct
  source 1..21: mol_type = other RNA
cagcggtgtcatctatgtgat

SEQ ID NO: 1009 - RNA, 21 nt, synthetic construct
  source 1..21: mol_type = other RNA
caggttttacatcactggaat

SEQ ID NO: 1010 - RNA, 21 nt, synthetic construct
  source 1..21: mol_type = other RNA
ccaactcttcaccctcaccaa

SEQ ID NO: 1011 - RNA, 19 nt, synthetic construct
  source 1..19: mol_type = other RNA
ccaccaactgtagatgtat

SEQ ID NO: 1012 - RNA, 19 nt, synthetic construct
  source 1..19: mol_type = other RNA
ccaccatgatcctgtcgat

SEQ ID NO: 1013 - RNA, 19 nt, synthetic construct
  source 1..19: mol_type = other RNA
cccaaaatcccaaactcaa

SEQ ID NO: 1014 - RNA, 19 nt, synthetic construct
  source 1..19: mol_type = other RNA
cccttctcagcggtgtcat

SEQ ID NO: 1015 - RNA, 21 nt, synthetic construct
  source 1..21: mol_type = other RNA
cctcctgttgctgagtatcat

SEQ ID NO: 1016 - RNA, 19 nt, synthetic construct
  source 1..19: mol_type = other RNA
cctacggtttcgcctacat

SEQ ID NO: 1017 - RNA, 19 nt, synthetic construct
  source 1..19: mol_type = other RNA
cctcctgttgctgagtatc

SEQ ID NO: 1018 - RNA, 21 nt, synthetic construct
  source 1..21: mol_type = other RNA
cctcctgttgctgagtatcat

SEQ ID NO: 1019 - RNA, 21 nt, synthetic construct
  source 1..21: mol_type = other RNA
cctcctgttgctgggtatcat

SEQ ID NO: 1020 - RNA, 19 nt, synthetic construct
  source 1..19: mol_type = other RNA
cctcgtgttgaatcttaaa

SEQ ID NO: 1021 - RNA, 21 nt, synthetic construct
  source 1..21: mol_type = other RNA
cctgtcgatcatcttcagcat

SEQ ID NO: 1022 - RNA, 21 nt, synthetic construct
  source 1..21: mol_type = other RNA
cctgttcttctgccaactctt

SEQ ID NO: 1023 - RNA, 19 nt, synthetic construct
  source 1..19: mol_type = other RNA
cctgttgctgagtatcatc

SEQ ID NO: 1024 - RNA, 21 nt, synthetic construct
  source 1..21: mol_type = other RNA
cgctgtttggccgggcagaaa

SEQ ID NO: 1025 - RNA, 19 nt, synthetic construct
  source 1..19: mol_type = other RNA
cggattactcctacggttt

SEQ ID NO: 1026 - RNA, 21 nt, synthetic construct
  source 1..21: mol_type = other RNA
cggtgtcatctatgtgatctt

SEQ ID NO: 1027 - RNA, 21 nt, synthetic construct
  source 1..21: mol_type = other RNA
cggtttataaaacctatttat

SEQ ID NO: 1028 - RNA, 21 nt, synthetic construct
  source 1..21: mol_type = other RNA
cgtcgcggtgctggtgctgct

SEQ ID NO: 1029 - RNA, 21 nt, synthetic construct
  source 1..21: mol_type = other RNA
cgtgatgagtgctgcggccat

SEQ ID NO: 1030 - RNA, 21 nt, synthetic construct
  source 1..21: mol_type = other RNA
ctaaggaactttacatcctaa

SEQ ID NO: 1031 - RNA, 21 nt, synthetic construct
  source 1..21: mol_type = other RNA
ctacggtttcgcctacatcct

SEQ ID NO: 1032 - RNA, 19 nt, synthetic construct
  source 1..19: mol_type = other RNA
ctatgtgatcttgcggaaa

SEQ ID NO: 1033 - RNA, 21 nt, synthetic construct
  source 1..21: mol_type = other RNA
ctcctacggtttcgcctacat

SEQ ID NO: 1034 - RNA, 21 nt, synthetic construct
  source 1..21: mol_type = other RNA
ctcctcctgttgctgagtatc

SEQ ID NO: 1035 - RNA, 21 nt, synthetic construct
  source 1..21: mol_type = other RNA
ctcctcctgttgctgagtatt

SEQ ID NO: 1036 - RNA, 19 nt, synthetic construct
  source 1..19: mol_type = other RNA
ctcctgttgctgagtatca

SEQ ID NO: 1037 - RNA, 21 nt, synthetic construct
  source 1..21: mol_type = other RNA
ctcctgttgctgagtatcatc

SEQ ID NO: 1038 - RNA, 21 nt, synthetic construct
  source 1..21: mol_type = other RNA
ctcctgttgctgagtatcatt

SEQ ID NO: 1039 - RNA, 21 nt, synthetic construct
  source 1..21: mol_type = other RNA
ctcggattactcctacggttt

SEQ ID NO: 1040 - RNA, 19 nt, synthetic construct
  source 1..19: mol_type = other RNA
ctctgttcctgttcttctg

SEQ ID NO: 1041 - RNA, 19 nt, synthetic construct
  source 1..19: mol_type = other RNA
ctggtgctgctgttcgtct

SEQ ID NO: 1042 - RNA, 19 nt, synthetic construct
  source 1..19: mol_type = other RNA
ctgtccaggccaccatgat

SEQ ID NO: 1043 - RNA, 19 nt, synthetic construct
  source 1..19: mol_type = other RNA
ctgttgattgaagatgtat

SEQ ID NO: 1044 - RNA, 19 nt, synthetic construct
  source 1..19: mol_type = other RNA
ctgttgctgagtatcatcg

SEQ ID NO: 1045 - RNA, 21 nt, synthetic construct
  source 1..21: mol_type = other RNA
ctgttgctgagtatcatcgtc

SEQ ID NO: 1046 - RNA, 19 nt, synthetic construct
  source 1..19: mol_type = other RNA
ctgtttggccgggcagaaa

SEQ ID NO: 1047 - RNA, 21 nt, synthetic construct
  source 1..21: mol_type = other RNA
ctttacatcctaacagtataa

SEQ ID NO: 1048 - RNA, 19 nt, synthetic construct
  source 1..19: mol_type = other RNA
gaaactccgctgagcagaa

SEQ ID NO: 1049 - RNA, 19 nt, synthetic construct
  source 1..19: mol_type = other RNA
gaaatgtccaccactgttt

SEQ ID NO: 1050 - RNA, 19 nt, synthetic construct
  source 1..19: mol_type = other RNA
gaagtagctaaggaacttt

SEQ ID NO: 1051 - RNA, 21 nt, synthetic construct
  source 1..21: mol_type = other RNA
gagccttaaagaagtagctaa

SEQ ID NO: 1052 - RNA, 19 nt, synthetic construct
  source 1..19: mol_type = other RNA
gaggaagggaaaacagaaa

SEQ ID NO: 1053 - RNA, 19 nt, synthetic construct
  source 1..19: mol_type = other RNA
gaggcacccggagtggcat

SEQ ID NO: 1054 - RNA, 21 nt, synthetic construct
  source 1..21: mol_type = other RNA
gagtatcatcgtcctccacgt

SEQ ID NO: 1055 - RNA, 19 nt, synthetic construct
  source 1..19: mol_type = other RNA
gatctctggcagaactgta

SEQ ID NO: 1056 - RNA, 21 nt, synthetic construct
  source 1..21: mol_type = other RNA
gcacccggagtggcatctcaa

SEQ ID NO: 1057 - RNA, 19 nt, synthetic construct
  source 1..19: mol_type = other RNA
gccttaaagaagtagctaa

SEQ ID NO: 1058 - RNA, 19 nt, synthetic construct
  source 1..19: mol_type = other RNA
gcggtgtcatctatgtgat

SEQ ID NO: 1059 - RNA, 19 nt, synthetic construct
  source 1..19: mol_type = other RNA
gctcctcctgttgctgagt

SEQ ID NO: 1060 - RNA, 21 nt, synthetic construct
  source 1..21: mol_type = other RNA
gctcctcctgttgctgagtat

SEQ ID NO: 1061 - RNA, 19 nt, synthetic construct
  source 1..19: mol_type = other RNA
gctggtctgtgcgtgatga

SEQ ID NO: 1062 - RNA, 19 nt, synthetic construct
  source 1..19: mol_type = other RNA
gctgttgattgaagatgta

SEQ ID NO: 1063 - RNA, 21 nt, synthetic construct
  source 1..21: mol_type = other RNA
ggaatcttccaaattcttgct

SEQ ID NO: 1064 - RNA, 19 nt, synthetic construct
  source 1..19: mol_type = other RNA
ggacacgcaactgatctct

SEQ ID NO: 1065 - RNA, 21 nt, synthetic construct
  source 1..21: mol_type = other RNA
ggcaatggacacgcaactgat

SEQ ID NO: 1066 - RNA, 21 nt, synthetic construct
  source 1..21: mol_type = other RNA
ggccaccaactgtagatgtat

SEQ ID NO: 1067 - RNA, 21 nt, synthetic construct
  source 1..21: mol_type = other RNA
ggccaccatgatcctgtcgat

SEQ ID NO: 1068 - RNA, 19 nt, synthetic construct
  source 1..19: mol_type = other RNA
ggctctgttcctgttcttc

SEQ ID NO: 1069 - RNA, 21 nt, synthetic construct
  source 1..21: mol_type = other RNA
gggaggaagggaaaacagaaa

SEQ ID NO: 1070 - RNA, 19 nt, synthetic construct
  source 1..19: mol_type = other RNA
gggcaggttttacatcact

SEQ ID NO: 1071 - RNA, 21 nt, synthetic construct
  source 1..21: mol_type = other RNA
gggggcaggttttacatcact

SEQ ID NO: 1072 - RNA, 19 nt, synthetic construct
  source 1..19: mol_type = other RNA
ggttttacatcactggaat

SEQ ID NO: 1073 - RNA, 21 nt, synthetic construct
  source 1..21: mol_type = other RNA
gtacatagtattgtttgcttt

SEQ ID NO: 1074 - RNA, 19 nt, synthetic construct
  source 1..19: mol_type = other RNA
gtatcatcgtcctccacgt

SEQ ID NO: 1075 - RNA, 19 nt, synthetic construct
  source 1..19: mol_type = other RNA
gtcgatcatcttcagcatt

SEQ ID NO: 1076 - RNA, 19 nt, synthetic construct
  source 1..19: mol_type = other RNA
gtgtcatctatgtgatctt

SEQ ID NO: 1077 - RNA, 21 nt, synthetic construct
  source 1..21: mol_type = other RNA
gttcctgttcttctgccaact

SEQ ID NO: 1078 - RNA, 21 nt, synthetic construct
  source 1..21: mol_type = other RNA
gttgaccatcagcctcgtgtt

SEQ ID NO: 1079 - RNA, 19 nt, synthetic construct
  source 1..19: mol_type = other RNA
gttgctgagtatcatcgtc

SEQ ID NO: 1080 - RNA, 19 nt, synthetic construct
  source 1..19: mol_type = other RNA
gtttataaaacctatttat

SEQ ID NO: 1081 - RNA, 21 nt, synthetic construct
  source 1..21: mol_type = other RNA
gttttacatcactggaatctt

SEQ ID NO: 1082 - RNA, 19 nt, synthetic construct
  source 1..19: mol_type = other RNA
tcaactcggattactccta

SEQ ID NO: 1083 - RNA, 19 nt, synthetic construct
  source 1..19: mol_type = other RNA
tcactggaatcttccaaat

SEQ ID NO: 1084 - RNA, 19 nt, synthetic construct
  source 1..19: mol_type = other RNA
tcagcattctgtctctgtt

SEQ ID NO: 1085 - RNA, 19 nt, synthetic construct
  source 1..19: mol_type = other RNA
tcatcatcaccaaacgaat

SEQ ID NO: 1086 - RNA, 21 nt, synthetic construct
  source 1..21: mol_type = other RNA
tccaccactgtttctcatcat

SEQ ID NO: 1087 - RNA, 19 nt, synthetic construct
  source 1..19: mol_type = other RNA
tcctcctgttgctgagtat

SEQ ID NO: 1088 - RNA, 21 nt, synthetic construct
  source 1..21: mol_type = other RNA
tcctcctgttgctgagtatca

SEQ ID NO: 1089 - RNA, 19 nt, synthetic construct
  source 1..19: mol_type = other RNA
tcctgttcttctgccaact

SEQ ID NO: 1090 - RNA, 19 nt, synthetic construct
  source 1..19: mol_type = other RNA
tcctgttgctgagtatcat

SEQ ID NO: 1091 - RNA, 21 nt, synthetic construct
  source 1..21: mol_type = other RNA
tcctgttgctgagtatcatcg

SEQ ID NO: 1092 - RNA, 19 nt, synthetic construct
  source 1..19: mol_type = other RNA
tcgcggtgctggtgctgct

SEQ ID NO: 1093 - RNA, 19 nt, synthetic construct
  source 1..19: mol_type = other RNA
tctcagcggtgtcatctat

SEQ ID NO: 1094 - RNA, 19 nt, synthetic construct
  source 1..19: mol_type = other RNA
tctctgttcctgttcttct

SEQ ID NO: 1095 - RNA, 19 nt, synthetic construct
  source 1..19: mol_type = other RNA
tgaccatcagcctcgtgtt

SEQ ID NO: 1096 - RNA, 19 nt, synthetic construct
  source 1..19: mol_type = other RNA
tgatgagtgctgcggccat

SEQ ID NO: 1097 - RNA, 21 nt, synthetic construct
  source 1..21: mol_type = other RNA
tgctggtgctgctgttcgtct

SEQ ID NO: 1098 - RNA, 21 nt, synthetic construct
  source 1..21: mol_type = other RNA
tgctgttgattgaagatgtat

SEQ ID NO: 1099 - RNA, 19 nt, synthetic construct
  source 1..19: mol_type = other RNA
tgctttgcattttctgatt

SEQ ID NO: 1100 - RNA, 19 nt, synthetic construct
  source 1..19: mol_type = other RNA
tgtcgatcatcttcagcat

SEQ ID NO: 1101 - RNA, 19 nt, synthetic construct
  source 1..19: mol_type = other RNA
tgtgtggactaagatgcat

SEQ ID NO: 1102 - RNA, 19 nt, synthetic construct
  source 1..19: mol_type = other RNA
tgttcttctgccaactctt

SEQ ID NO: 1103 - RNA, 21 nt, synthetic construct
  source 1..21: mol_type = other RNA
tgttgctgagtatcatcgtcc

SEQ ID NO: 1104 - RNA, 19 nt, synthetic construct
  source 1..19: mol_type = other RNA
ttacatcctaacagtataa

SEQ ID NO: 1105 - RNA, 19 nt, synthetic construct
  source 1..19: mol_type = other RNA
ttacccagaaataagataa

SEQ ID NO: 1106 - RNA, 19 nt, synthetic construct
  source 1..19: mol_type = other RNA
ttgctgagtatcatcgtcc

SEQ ID NO: 1107 - RNA, 19 nt, synthetic construct
  source 1..19: mol_type = other RNA
ttgctggtctgtgcgtgat

SEQ ID NO: 1108 - RNA, 21 nt, synthetic construct
  source 1..21: mol_type = other RNA
ttgctgttgattgaagatgta

SEQ ID NO: 1109 - RNA, 19 nt, synthetic construct
  source 1..19: mol_type = other RNA
tttacatcactggaatctt

SEQ ID NO: 1110 - RNA, 21 nt, synthetic construct
  source 1..21: mol_type = other RNA
tttgctttgcattttctgatt

SEQ ID NO: 1111 - RNA, 21 nt, synthetic construct
  source 1..21: mol_type = other RNA
ttttacccagaaataagataa

SEQ ID NO: 1112 - RNA, 23 nt, synthetic construct
  source 1..23: mol_type = other RNA
aaacagtggtggacatttcctga

SEQ ID NO: 1113 - RNA, 23 nt, synthetic construct
  source 1..23: mol_type = other RNA
aaaccgtaggagtaatccgagtt

SEQ ID NO: 1114 - RNA, 23 nt, synthetic construct
  source 1..23: mol_type = other RNA
aaagcaaacaatactatgtacat

SEQ ID NO: 1115 - RNA, 23 nt, synthetic construct
  source 1..23: mol_type = other RNA
aaagttccttagctacttcttta

SEQ ID NO: 1116 - RNA, 23 nt, synthetic construct
  source 1..23: mol_type = other RNA
aacacgaggctgatggtcaacat

SEQ ID NO: 1117 - RNA, 23 nt, synthetic construct
  source 1..23: mol_type = other RNA
aagagttggcagaagaacaggaa

SEQ ID NO: 1118 - RNA, 23 nt, synthetic construct
  source 1..23: mol_type = other RNA
aagatcacatagatgacaccgct

SEQ ID NO: 1119 - RNA, 23 nt, synthetic construct
  source 1..23: mol_type = other RNA
aagattccagtgatgtaaaacct

SEQ ID NO: 1120 - RNA, 23 nt, synthetic construct
  source 1..23: mol_type = other RNA
aagttctgctcagcggagtttct

SEQ ID NO: 1121 - RNA, 23 nt, synthetic construct
  source 1..23: mol_type = other RNA
aatactcagcaacaggaggagca

SEQ ID NO: 1122 - RNA, 23 nt, synthetic construct
  source 1..23: mol_type = other RNA
aatcagaaaatgcaaagcaaaaa

SEQ ID NO: 1123 - RNA, 23 nt, synthetic construct
  source 1..23: mol_type = other RNA
aatgatactcagcaacaggagga

SEQ ID NO: 1124 - RNA, 23 nt, synthetic construct
  source 1..23: mol_type = other RNA
aatttggaagattccagtgatgt

SEQ ID NO: 1125 - RNA, 23 nt, synthetic construct
  source 1..23: mol_type = other RNA
acagactgcagccattcgtttgg

SEQ ID NO: 1126 - RNA, 23 nt, synthetic construct
  source 1..23: mol_type = other RNA
acgtggaggacgatgatactcag

SEQ ID NO: 1127 - RNA, 23 nt, synthetic construct
  source 1..23: mol_type = other RNA
actcagcaacaggaggagcattc

SEQ ID NO: 1128 - RNA, 23 nt, synthetic construct
  source 1..23: mol_type = other RNA
agacgaacagcagcaccagcacc

SEQ ID NO: 1129 - RNA, 23 nt, synthetic construct
  source 1..23: mol_type = other RNA
agagatcagttgcgtgtccattc

SEQ ID NO: 1130 - RNA, 23 nt, synthetic construct
  source 1..23: mol_type = other RNA
agcaagaatttggaagattccag

SEQ ID NO: 1131 - RNA, 23 nt, synthetic construct
  source 1..23: mol_type = other RNA
agcagcaccagcaccgcgacgtg

SEQ ID NO: 1132 - RNA, 23 nt, synthetic construct
  source 1..23: mol_type = other RNA
aggatgtaggcgaaaccgtagga

SEQ ID NO: 1133 - RNA, 23 nt, synthetic construct
  source 1..23: mol_type = other RNA
agtgatgtaaaacctgcccccct

SEQ ID NO: 1134 - RNA, 23 nt, synthetic construct
  source 1..23: mol_type = other RNA
agttggcagaagaacaggaacag

SEQ ID NO: 1135 - RNA, 23 nt, synthetic construct
  source 1..23: mol_type = other RNA
ataaataggttttataaaccgga

SEQ ID NO: 1136 - RNA, 23 nt, synthetic construct
  source 1..23: mol_type = other RNA
atacatctacagttggtggccaa

SEQ ID NO: 1137 - RNA, 23 nt, synthetic construct
  source 1..23: mol_type = other RNA
atacatcttcaatcaacagcaac

SEQ ID NO: 1138 - RNA, 23 nt, synthetic construct
  source 1..23: mol_type = other RNA
atactcagcaacaggaggagcat

SEQ ID NO: 1139 - RNA, 23 nt, synthetic construct
  source 1..23: mol_type = other RNA
atcacatagatgacaccgctgag

SEQ ID NO: 1140 - RNA, 23 nt, synthetic construct
  source 1..23: mol_type = other RNA
atcagttgcgtgtccattgccca

SEQ ID NO: 1141 - RNA, 23 nt, synthetic construct
  source 1..23: mol_type = other RNA
atcgacaggatcatggtggcctg

SEQ ID NO: 1142 - RNA, 23 nt, synthetic construct
  source 1..23: mol_type = other RNA
atcttagtccacacagttggtat

SEQ ID NO: 1143 - RNA, 23 nt, synthetic construct
  source 1..23: mol_type = other RNA
atgatacccagcaacaggaggag

SEQ ID NO: 1144 - RNA, 23 nt, synthetic construct
  source 1..23: mol_type = other RNA
atgatactcagcaacaggaggag

SEQ ID NO: 1145 - RNA, 23 nt, synthetic construct
  source 1..23: mol_type = other RNA
atgatgagaaacagtggtggaca

SEQ ID NO: 1146 - RNA, 23 nt, synthetic construct
  source 1..23: mol_type = other RNA
atgcatcttagtccacacagttg

SEQ ID NO: 1147 - RNA, 23 nt, synthetic construct
  source 1..23: mol_type = other RNA
atgctgaagatgatcgacaggat

SEQ ID NO: 1148 - RNA, 23 nt, synthetic construct
  source 1..23: mol_type = other RNA
atggccgcagcactcatcacgca

SEQ ID NO: 1149 - RNA, 23 nt, synthetic construct
  source 1..23: mol_type = other RNA
atgtaggcgaaaccgtaggagta

SEQ ID NO: 1150 - RNA, 23 nt, synthetic construct
  source 1..23: mol_type = other RNA
attccagtgatgtaaaacctgcc

SEQ ID NO: 1151 - RNA, 23 nt, synthetic construct
  source 1..23: mol_type = other RNA
cgatgatactcagcaacaggagg

SEQ ID NO: 1152 - RNA, 23 nt, synthetic construct
  source 1..23: mol_type = other RNA
gacgatgatactcagcaacagga

SEQ ID NO: 1153 - RNA, 23 nt, synthetic construct
  source 1..23: mol_type = other RNA
gatactcagcaacaggaggagca

SEQ ID NO: 1154 - RNA, 23 nt, synthetic construct
  source 1..23: mol_type = other RNA
gatgatactcagcaacaggagga

SEQ ID NO: 1155 - RNA, 23 nt, synthetic construct
  source 1..23: mol_type = other RNA
ggacgatgatactcagcaacagg

SEQ ID NO: 1156 - RNA, 23 nt, synthetic construct
  source 1..23: mol_type = other RNA
tacatcttcaatcaacagcaacc

SEQ ID NO: 1157 - RNA, 23 nt, synthetic construct
  source 1..23: mol_type = other RNA
tgatactcagcaacaggaggagc

SEQ ID NO: 1158 - RNA, 23 nt, synthetic construct
  source 1..23: mol_type = other RNA
ttagctacttctttaaggctcaa

SEQ ID NO: 1159 - RNA, 23 nt, synthetic construct
  source 1..23: mol_type = other RNA
ttaggatgtaaagttccttagct

SEQ ID NO: 1160 - RNA, 23 nt, synthetic construct
  source 1..23: mol_type = other RNA
ttatactgttaggatgtaaagtt

SEQ ID NO: 1161 - RNA, 23 nt, synthetic construct
  source 1..23: mol_type = other RNA
ttatcttatttctgggtaaaaca

SEQ ID NO: 1162 - RNA, 23 nt, synthetic construct
  source 1..23: mol_type = other RNA
ttctgctcagcggagtttctgcc

SEQ ID NO: 1163 - RNA, 23 nt, synthetic construct
  source 1..23: mol_type = other RNA
ttgagatgccactccgggtgcct

SEQ ID NO: 1164 - RNA, 23 nt, synthetic construct
  source 1..23: mol_type = other RNA
ttgagtttgggattttgggctcg

SEQ ID NO: 1165 - RNA, 23 nt, synthetic construct
  source 1..23: mol_type = other RNA
ttggtgagggtgaagagttggca

SEQ ID NO: 1166 - RNA, 23 nt, synthetic construct
  source 1..23: mol_type = other RNA
tttccgcaagatcacatagatga

SEQ ID NO: 1167 - RNA, 23 nt, synthetic construct
  source 1..23: mol_type = other RNA
tttctgcccggccaaacagcgta

SEQ ID NO: 1168 - RNA, 23 nt, synthetic construct
  source 1..23: mol_type = other RNA
tttctgttttcccttcctccctt

SEQ ID NO: 1169 - RNA, 23 nt, synthetic construct
  source 1..23: mol_type = other RNA
tttggtttgagtttgggattttg

SEQ ID NO: 1170 - DNA, 1828 bp, Homo sapiens
  source 1..1828: mol_type = genomic DNA
agttacagggagcaccaccagggaacatctcggggagcctggttggaagctgcaggcttagtctgtcggctgcgggtctctgactgccctgtggggagggtcttgccttaacatcccttgcatttggctgcaaagaaatctgcttggaagaaggggttacgctgtttggccgggcagaaactccgctgagcagaacttgccgccagaatgctcctcctgttgctgagtatcatcgtcctccacgtcgcggtgctggtgctgctgttcgtctccacgatcgtcagccaatggatcgtgggcaatggacacgcaactgatctctggcagaactgtagcacctcttcctcaggaaatgtccaccactgtttctcatcatcaccaaacgaatggctgcagtctgtccaggccaccatgatcctgtcgatcatcttcagcattctgtctctgttcctgttcttctgccaactcttcaccctcaccaaggggggcaggttttacatcactggaatcttccaaattcttgctggtctgtgcgtgatgagtgctgcggccatctacacggtgaggcacccggagtggcatctcaactcggattactcctacggtttcgcctacatcctggcctgggtggccttccccctggcccttctcagcggtgtcatctatgtgatcttgcggaaacgcgaatgaggcgcccagacggtctgtctgaggctctgagcgtacatagggaagggaggaagggaaaacagaaagcagacaaagaaaaaagagctagcccaaaatcccaaactcaaaccaaaccaaacagaaagcagtggaggtgggggttgctgttgattgaagatgtatataatatctccggtttataaaacctatttataacactttttacatatatgtacatagtattgtttgctttttatgttgaccatcagcctcgtgttgagccttaaagaagtagctaaggaactttacatcctaacagtataatccagctcagtatttttgttttgttttttgtttgtttgttttgttttacccagaaataagataactccatctcgccccttccctttcatctgaaagaagatacctccctcccagtccacctcatttagaaaaccaaagtgtgggtagaaaccccaaatgtccaaaagcccttttctggtgggtgacccagtgcatccaacagaaacagccgctgcccgaacctctgtgtgaagctttacgcgcacacggacaaaatgcccaaactggagcccttgcaaaaacacggcttgtggcattggcatacttgcccttacaggtggagtatcttcgtcacacatctaaatgagaaatcagtgacaacaagtctttgaaatggtgctatggatttaccattccttattatcactaatcatctaaacaactcactggaaatccaattaacaattttacaacataagatagaatggagacctgaataattctgtgtaatataaatggtttataactgcttttgtacctagctaggctgctattattactataatgagtaaatcataaagccttcatcactcccacatttttcttacggtcggagcatcagaacaagcgtctagactccttgggaccgtgagttcctagagcttggctgggtctaggctgttctgtgcctccaaggactgtctggcaatgacttgtattggccaccaactgtagatgtatatatggtgcccttctgatgctaagactccagaccttttgtttttgctttgcattttctgattttataccaactgtgtggactaagatgcattaaaataaacatcagagtaactca